(19) United States
(12) Patent Application Publication
(10) Pub. No.: US 2005/0203280 A1
(43) Pub. Date: Sep. 15, 2005

(54) Alloiococcus otitidis open reading frames (orfs) encoding polypeptide antigens, immunogenic compositions and uses thereof

(76) Inventors: John Calhoun McMichael, Rochester, NY (US); Robert John Zagursky, Victor, NY (US); Leah Diane Fletcher, Geneseo, NY (US)

Correspondence Address: WYETH;PATENT LAW GROUP, 5 GIRALDA FARMS, MADISON, NJ, 07940, US

(21) Appl. No.: 10/501,282

(22) PCT Filed: Nov. 25, 2002
(86) PCT No.: PCT/US02/36123

§ 371 Date: Jul. 9, 2004

(30) Foreign Application Priority Data
Nov. 29, 2001 (US) 60333777
Nov. 18, 2002 (US) 60426742

(51) Int. Cl. C12Q 1/68; C07H 21/04; C07K 14/195

(52) U.S. Cl. 530/350; 435/6; 435/69.1; 435/252.3; 435/320.1; 536/23.7

(57) ABSTRACT

The present invention relates to the complete genomic sequence of Gram-positive bacterium, Alloiococcus otitidis. The present invention also relates to polynucleotide sequences encoding polypeptides of Alloiococcus otitidis. In particular, the invention relates to antigenic polypeptides encoded by the Alloiococcus otitidis open reading frames (ORFs), and to their use in immunogenic compositions, therapeutics, diagnostics and the like.

### FIELD OF THE INVENTION

The present invention relates to the genomic sequence of Alloiococcus otitidis and polynucleotide sequences encoding polypeptides of the Gram-positive bacterium, Alloiococcus otitidis. The invention also relates to polynucleotides and polynucleotides encoding polypeptides, preferably antigenic polypeptides, encoded by the Alloiococcus otitidis open reading frames and the uses thereof.

### BACKGROUND OF THE INVENTION

Otitis media, an inflammation of the middle ear, is the most frequent cause of visits to pediatricians' offices in the United States (Schappert, 1991). Approximately 80% of all children experience at least one episode of Otitis media by the age of three (Klein, 1994). There are three main types of otitis media: Acute otitis media (AOM), otorrhea, and otitis media with effusion (OME). Alloiococcus otitidis has only been associated with otitis media with effusion (OME), but this may be due to the difficulty of its detection by standard bacterial culturing methods. Its detection in the effusions is likely due to the fact that the effusions are normally sterile and few or no competing bacterial species are isolated from them. Without the interference of a faster growing nasophryngeal species, the culture plates are incubated for the longer duration needed to detect Alloiococcus otitidis colonies.

Three other bacterial species are commonly isolated from middle ear effusions. These are nontypable Haemophilus influenzae, Moraxella catarrhalis, and Streptococcus pneumoniae. One or more of these species have been found in one study to be associated with about 77% of all cases of OME using a PCR detection method (Post, 2000). This study did not include assaying for Alloiococcus otitidis, so a portion of the unaccounted cases may be due to this organism.

The bacterium Alloiococcus otitidis was first isolated from the middle ear fluids of 10 children in the Buffalo, N.Y. area with persistent OME and characterized as a large catalase negative, Gram-positive cocci that tend to occur in clumps, often in tetrads. It is slow growing and requires 2 to 5 days at 37° C. before colonies are seen on sheep blood agar plates (Table 1). The bacterium was named Alloiococcus otitis by Aguirre and Collins (1992), who showed that it was different from other known Gram-positive species based on its 16S rRNA sequence. Recently the bacterium's name has been changed from Alloiococcus otitis to Alloiococcus otitidis (Hendolin, et al. Pediatr. Infect. Dis. J. 18:860 (1999), Hendolin et al. J. Clin. Microbiol. 38:125 (2000)).

Several studies of the epidemiology Alloiococcus otitidis indicate it is associated with otitis media with effusion. These are summarized in Table 1. These studies have been carried out using both culture and PCR techniques. The number of cases detected by culture, as might be expected from the fastidious growth requirements of the bacterium, was less than the number detected by PCR. Assuming that the bacterium is detected more accurately by the PCR method, the bacterium is detected in between 10 and 50% of patients with OME. This frequency suggests that this organism represents a significant public health problem. Consequently, there is a need for immunogenic compositions to prevent symptoms caused by Alloiococcus otitidis infections. There is also a need for compositions for diagnosing Alloiococcus otitidis infections.

TABLE 1
SUMMARY OF STUDIES INDICATING AN ASSOCIATION
OF ALLOIOCOCCUS OTITIDIS
WITH OTITIS MEDIA WITH EFFUSION (OME).

| % Detected | N^{a} | Method | Reference |
|---|---|---|---|
| 8 | 200 | Culture | Faden & Dryja, J. Clin. |
| | | | Microbiol. 27: 2488 (1989) |
| 3 | 100 | Culture | Sih et al., ICAAC (1992) |
| 20 | 25 | PCR | Hendolin et al., J. Clin. Microbiol. |
| | | | 35: 2854 (1997) |
| 50 | 12 | PCR | Beswick, et al. Lancet |
| | | | 345: 386 (1999) |
| 42 | 67 | PCR | Hendolin, et al. Pediatr. Infect. |
| | | | Dis. J. 18: 860 (1999) |
| 10 | 49 | PCR | Hendolin et al. J. Clin. |
| | | | Microbiol. 38: 125 (2000) |

^{a}Number of persons in study.

### SUMMARY OF THE INVENTION

The present invention broadly relates to Alloiococcus otitidis genomic sequence. Particularly, the invention relates to newly identified polynucleotide open reading frames (ORFs) comprised within the genomic nucleotide sequence of Alloiococcus otitidis, and to polypeptides encoded by the ORFs. More particularly, the ORFs encode polypeptides that are surface localized, secreted, or exposed on Alloiococcus otitidis.

Thus, in certain aspects, the invention relates to Alloiococcus otitidis ORFs that encode Alloiococcus otitidis polypeptides. In preferred embodiments, these Alloiococcus otitidis polypeptides are antigenic polypeptides. As defined hereinafter, an Alloiococcus otitidis antigenic polypeptide, antigen or immunogen, is an Alloiococcus otitidis polypeptide that is immunoreactive with an antibody or an Alloiococcus otitidis polypeptide that elicits an immune response. In other embodiments, the invention relates to the polynucleotides encoding these antigenic polypeptides. In other aspects, the invention relates to vectors comprising Alloiococcus otitidis ORFs and cells or animals transformed with these vectors. The invention relates also to methods of detecting these nucleic acids or polypeptides and kits for diagnosing Alloiococcus otitidis infection. The invention further relates to pharmaceutical compositions, in particular immunogenic compositions, for the prevention and/or treatment of bacterial infection, in particular Alloiococcus otitidis.

In a preferred embodiment, the immunogenic compositions are used for the treatment or prevention of non-systemic diseases, particularly of the Otitis media, which are induced or worsened by Alloiococcus otitidis.

In particular embodiments, the invention relates to an isolated polynucleotide of an Alloiococcus otitidis genomic sequence, wherein the polynucleotide comprises a nucleotide sequence having at least about 70% identity to a nucleotide sequence chosen from one of odd numbered sequences set out in SEQ ID NO: 1 through 6649, a degenerate variant thereof, or a fragment thereof. As defined hereinafter, a “degenerate variant” is defined as a polynucleotide that differs from the nucleotide sequence shown in SEQ ID NO:1 through SEQ ID NO:6649 (and fragments thereof) due to degeneracy of the genetic code, but still encodes the same Alloiococcus otitidis polypeptide (i.e., SEQ ID NO:2 through SEQ ID NO:6650) as that encoded by the nucleotide sequence shown in SEQ ID NO:1 through SEQ ID NO:6649.

In other embodiments, the polynucleotide is a complement to a nucleotide sequence chosen from one of the odd numbered sequences set out in SEQ ID NO: 1 through SEQ ID NO: 6649, a degenerate variant thereof, or a fragment thereof. In yet other embodiments, the polynucleotide is selected from the group consisting of genomic DNA, cDNA and RNA or may further comprise heterologous nucleotides.

In another embodiment, the invention comprises an isolated polynucleotide that hybridizes to a nucleotide sequence chosen from one of odd numbered sequences set out in SEQ ID NO: 1 through SEQ ID NO: 6649, a complement hereof, a degenerate variant thereof, or a fragment thereof, under high stringency hybridization conditions. In yet other embodiments, the polynucleotide hybridizes under intermediate stringency hybridization conditions.

In a preferred embodiment, an isolated polynucleotide of a an Alloiococcus otitidis genomic sequence comprises a nucleotide sequence chosen from one of odd numbered sequences set out in SEQ ID NO: 1 through SEQ ID NO: 6649, a fragment thereof, or a degenerate variant thereof, and encodes a polypeptide, a biological equivalent thereof, or a fragment thereof, selected from the group consisting of (a) an Alloiococcus otitidis polypeptide predicted by SignaIP algorithm analysis as having met all four criteria set out in the SignaIP algorithm for a polypeptide having a signal peptide and a subset of these are predicted by HMM SignaIP algorithm analysis as having a signal peptide; (b) an Alloiococcus otitidis polypeptide predicted by SignaIP algorithm analysis as having met three of the four criteria set out in the SignaIP algorithm for a polypeptide having a signal peptide and a subset of these predicted by HMM SignaIP algorithm analysis as having a signal peptide; (c) an Alloiococcus otitidis polypeptide predicted by HMM SignaIP algorithm analysis as having a signal peptide; (d) an Alloiococcus otitidis polypeptide predicted by HMM SignaIP algorithm analysis as being a non-secretory protein; (e) an Alloiococcus otitidis polypeptide identified by Pfam analysis; (f) an Alloiococcus otitidis polypeptide identified by BlastP analysis as being secreted, surface exposed, vaccine candidate; (g) an Alloiococcus otitidis polypeptide identified using proteomics methodology as sharing homology with putative surface exposed proteins of Streptococcus pneumoniae; (h) an Alloiococcus otitidis polypeptide having a LPXTG motif, wherein the polypeptide is covalently attached to the peptidoglycan layer; (i) an Alloiococcus otitidis lipoprotein; (j) an Alloiococcus otitidis polypeptide non-covalently associated with the peptidoglycan layer; (k) an Alloiococcus otitidis polypeptide having an RGDX motif wherein X is not a proline residue; (I) an Alloiococcus otitidis polypeptide predicted by BlastP as being involved in capsule biosynthesis and transport; (m) an Alloiococcus otitidis polypeptide identified by BlastP as being localized within the capsule loci of the Alloiococcus otitidis genome; (n) an Alloiococcus otitidis polypeptide predicted by BlastP as being associated with sporulation; (o) an Alloiococcus otitidis polypeptide encoded by unique ORFs identified by BlastP analysis as having a BlastP ‘E Value’ of >e⁻¹⁰; (p) an Alloiococcus otitidis polypeptide identified by Glimmer™ ORF finder program; (q) an Alloiococcus otitidis polypeptide identified by GeneMark™ ORF finder program; (r) an Alloiococcus otitidis polypeptide identified by Applicants' assignee's ORF finder program that searches for an ATG, GTG or TTG Start codon between a Stop-Stop ORF; and (s) an Alloiococcus otitidis polypeptide identified by Applicants' assignee's ORF finder program that searches for a transmembrane domain between two Stop codons and a Start codon immediately upstream of this transmembrane region.

In one aspect of this embodiment, the polynucleotide is a complement to a nucleotide sequence selected from one of odd numbered sequences set out in SEQ ID NO: 1 and SEQ ID NO: 6649, a degenerate variant thereof, or a fragment thereof. In yet other aspects of this embodiment, the polynucleotide is selected from the group consisting of DNA, chromosomal DNA, cDNA and RNA or may further comprise heterologous nucleotides. In another aspect of the invention, the polynucleotide encodes a fusion polypeptide.

In a preferred embodiment, the invention comprises an isolated polynucleotide of the Alloiococcus otitidis genomic sequence encoding a polypeptide, which is predicted by SignaIP algorithm analysis as having met all four criteria set out in the SignaIP algorithm for a polypeptide having a signal peptide, and the polynucleotide is selected from one of Seq. ID No. 89, Seq. ID No. 127, Seq. ID No.131, Seq. ID No.133, Seq. ID No. 161, Seq. ID No.163, Seq. ID No. 213, Seq. ID No. 215, Seq. ID No. 225, Seq. ID No. 249, Seq. ID No. 251, Seq. ID No. 287, Seq. ID No. 289, Seq. ID No. 369, Seq. ID No. 371, Seq. ID No. 375, Seq. ID No. 377, Seq. ID No. 497, Seq. ID No. 503, Seq. ID No. 505, Seq. ID No. 527, Seq. ID No. 549, Seq. ID No. 575, Seq. ID No. 617, Seq. ID No. 657, Seq. ID No. 659, Seq. ID No. 661, Seq. ID No. 675, Seq. ID No. 715, Seq. ID No. 717, Seq. ID No. 745, Seq. ID No. 747, Seq. ID No. 755, Seq. ID No. 757, Seq. ID No. 759, Seq. ID No. 761, Seq. ID No. 773, Seq. ID No. 807, Seq. ID No. 967, Seq. ID No. 973, Seq. ID No. 975, Seq. ID No. 1013, Seq. ID No. 1025, Seq. ID No. 1027, Seq. ID No. 1039, Seq. ID No. 1075, Seq. ID No. 1089, Seq. ID No. 1195, Seq. ID No. 1197, Seq. ID No. 1207, Seq. ID No. 1239, Seq. ID No. 1243, Seq. ID No. 1245, Seq. ID No. 1269, Seq. ID No. 1315, Seq. ID No. 1317, Seq. ID No. 1331, Seq. ID No. 1365, Seq. ID No. 1367, Seq. ID No. 1369, Seq. ID No. 1445, Seq. ID No. 1447, Seq. ID No. 1513, Seq. ID No. 1517, Seq. ID No. 1545, Seq. ID No. 1549, Seq. ID No. 1561, Seq. ID No. 1569, Seq. ID No. 1571, Seq. ID No. 1585, Seq. ID No. 1643, Seq. ID No. 1653, Seq. ID No. 1661, Seq. ID No. 1663, Seq. ID No. 1707, Seq. ID No. 1733, Seq. ID No. 1737, Seq. ID No. 1755, Seq. ID No. 1765, Seq. ID No. 1767, Seq. ID No. 1775, Seq. ID No. 1791, Seq. ID No. 1805, Seq. ID No. 1875, Seq. ID No. 1969, Seq. ID No. 1981, Seq. ID No. 2053, Seq. ID No. 2055, Seq. ID No. 2087, Seq. ID No. 2089, Seq. ID No. 2091, Seq. ID No. 2105, Seq. ID No. 2115, Seq. ID No. 2149, Seq. ID No. 2151, Seq. ID No. 2159, Seq. ID No. 2177, Seq. ID No. 2277, Seq. ID No. 2293, Seq. ID No. 2321, Seq. ID No. 2347, Seq. ID No. 2351, Seq. ID No. 2353, Seq. ID No. 2365, Seq. ID No. 2395, Seq. ID No. 2421, Seq. ID No. 2425, Seq. ID No. 2427, Seq. ID No. 2429, Seq. ID No. 2449, Seq. ID No. 2459, Seq. ID No. 2525, Seq. ID No. 2531, Seq. ID No. 2607, Seq. ID No. 2651, Seq. ID No. 2679, Seq. ID No. 2693, Seq. ID No. 2699, Seq. ID No. 2703, Seq. ID No. 2753, Seq. ID No. 2785, Seq. ID No. 2813, Seq. ID No. 2829, Seq. ID No. 2835, Seq. ID No. 2841, Seq. ID No. 2879, Seq. ID No. 2881, Seq. ID No. 2935, Seq. ID No. 2937, Seq. ID No. 2949, Seq. ID No. 2965, Seq. ID No. 2967, Seq. ID No. 2973, Seq. ID No. 3007, Seq. ID No. 3009, Seq. ID No. 3011, Seq. ID No. 3029, Seq. ID No. 3031, Seq. ID No. 3109, Seq. ID No. 3111, Seq. ID No. 3219, Seq. ID No. 3223, Seq. ID No. 3273, Seq. ID No. 3297, Seq. ID No. 3331, Seq. ID No. 3335, Seq. ID No. 3341, Seq. ID No. 3349, Seq. ID No. 3353, Seq. ID No. 3355, Seq. ID No. 3377, Seq. ID No. 3391, Seq. ID No. 3425, Seq. ID No. 3449, Seq. ID No. 3493, Seq. ID No. 3573, Seq. ID No. 3611, Seq. ID No. 3619, Seq. ID No. 3689, Seq. ID No. 3753, Seq. ID No. 3755, Seq. ID No. 3765, Seq. ID No. 3781, Seq. ID No. 3783, Seq. ID No. 3851, Seq. ID No. 3903, Seq. ID No. 4005, Seq. ID No. 4007, Seq. ID No. 4027, Seq. ID No. 4073, Seq. ID No. 4137, Seq. ID No. 4141, Seq. ID No. 4143, Seq. ID No. 4145, Seq. ID No. 4225, Seq. ID No. 4343, Seq. ID No. 4385, Seq. ID No. 4399, Seq. ID No. 4469, Seq. ID No. 4483, Seq. ID No. 4493, Seq. ID No. 4495, Seq. ID No. 4533, Seq. ID No. 4699, Seq. ID No. 4709, Seq. ID No. 4711, Seq. ID No. 4735, Seq. ID No. 4827, Seq. ID No. 4871, Seq. ID No. 4873, Seq. ID No. 4879, Seq. ID No. 4921, Seq. ID No. 4981, Seq. ID No. 5115, Seq. ID No. 5267, Seq. ID No. 5341, Seq. ID No. 5387, Seq. ID No. 5465, Seq. ID No. 5467, Seq. ID No. 5479, Seq. ID No. 5497, Seq. ID No. 5513, Seq. ID No. 5543, Seq. ID No. 5545, Seq. ID No. 5567, Seq. ID No. 5587, Seq. ID No. 5589, Seq. ID No. 5591, Seq. ID No. 5611, Seq. ID No. 5621, Seq. ID No. 5641, Seq. ID No. 5681, Seq. ID No. 5719, Seq. ID No. 5771, Seq. ID No. 5783, Seq. ID No. 5895, Seq. ID No. 5899, Seq. ID No. 5901, Seq. ID No. 5927, Seq. ID No. 5929, Seq. ID No. 5939, Seq. ID No. 5987, Seq. ID No. 6013, Seq. ID No. 6087, Seq. ID No. 6089, Seq. ID No. 6091, Seq. ID No. 6093, Seq. ID No. 6095, Seq. ID No. 6103, Seq. ID No. 6169, Seq. ID No. 6185, Seq. ID No. 6187, Seq. ID No. 6195, Seq. ID No. 6197, Seq. ID No. 6203, Seq. ID No. 6205, Seq. ID No. 6221, Seq. ID No. 6223, Seq. ID No. 6229, Seq. ID No. 6291, Seq. ID No. 6415, Seq. ID No. 6417, Seq. ID No. 6473, Seq. ID No. 6507, Seq. ID No. 6543, Seq. ID No. 6595, Seq. ID No. 6597, Seq. ID No. 6599, Seq. ID No. 6601, and Seq. ID No. 6603.

In yet another preferred embodiment, the invention comprises an isolated polynucleotide of the Alloiococcus otitidis genomic sequence encoding a polypeptide which is predicted by SignaIP algorithm analysis as having met three of the four criteria set out in the SignaIP algorithm for a polypeptide having a signal peptide and which is predicted by HMM Signal P algorithm analysis as having a signal peptide, and the polynucleotide is selected from one of Seq. ID No. 7, Seq. ID No. 11, Seq. ID No. 23, Seq. ID No. 45, Seq. ID No. 47, Seq. ID No. 53, Seq. ID No. 59, Seq. ID No. 65, Seq. ID No. 67, Seq. ID No. 87, Seq. ID No. 91, Seq. ID No. 139, Seq. ID No. 151, Seq. ID No. 159, Seq. ID No. 165, Seq. ID No. 169, Seq. ID No. 185, Seq. ID No. 211, Seq. ID No. 217, Seq. ID No. 235, Seq. ID No. 237, Seq. ID No. 239, Seq. ID No. 271, Seq. ID No. 285, Seq. ID No. 291, Seq. ID No. 327, Seq. ID No. 349, Seq. ID No. 385, Seq. ID No. 387, Seq. ID No. 407, Seq. ID No. 427, Seq. ID No. 431, Seq. ID No. 455, Seq. ID No. 457, Seq. ID No. 495, Seq. ID No. 499, Seq. ID No. 511, Seq. ID No. 513, Seq. ID No. 515, Seq. ID No. 525, Seq. ID No. 545, Seq. ID No. 547, Seq. ID No. 557, Seq. ID No. 569, Seq. ID No. 589, Seq. ID No. 591, Seq. ID No. 593, Seq. ID No. 595, Seq. ID No. 609, Seq. ID No. 637, Seq. ID No. 639, Seq. ID No. 641, Seq. ID No. 653, Seq. ID No. 669, Seq. ID No. 679, Seq. ID No. 719, Seq. ID No. 723, Seq. ID No. 739, Seq. ID No. 749, Seq. ID No. 751, Seq. ID No. 763, Seq. ID No. 765, Seq. ID No. 769, Seq. ID No. 777, Seq. ID No. 779, Seq. ID No. 781, Seq. ID No. 819, Seq. ID No. 895, Seq. ID No. 903, Seq. ID No. 905, Seq. ID No. 909, Seq. ID No. 935, Seq. ID No. 937, Seq. ID No. 951, Seq. ID No. 955, Seq. ID No. 979, Seq. ID No. 981, Seq. ID No. 983, Seq. ID No. 985, Seq. ID No. 1005, Seq. ID No. 1007, Seq. ID No. 1009, Seq. ID No. 1015, Seq. ID No. 1019, Seq. ID No. 1029, Seq. ID No. 1031, Seq. ID No. 1047, Seq. ID No. 1091, Seq. ID No. 1111, Seq. ID No. 1137, Seq. ID No. 1151, Seq. ID No. 1153, Seq. ID No. 1169, Seq. ID No. 1173, Seq. ID No. 1183, Seq. ID No. 1191, Seq. ID No. 1193, Seq. ID No. 1213, Seq. ID No. 1223, Seq. ID No. 1229, Seq. ID No. 1247, Seq. ID No. 1259, Seq. ID No. 1283, Seq. ID No. 1307, Seq. ID No. 1319, Seq. ID No. 1321, Seq. ID No. 1327, Seq. ID No. 1341, Seq. ID No. 1343, Seq. ID No. 1347, Seq. ID No. 1389, Seq. ID No. 1401, Seq. ID No. 1429, Seq. ID No. 1479, Seq. ID No. 1551, Seq. ID No. 1559, Seq. ID No. 1579, Seq. ID No. 1587, Seq. ID No. 1621, Seq. ID No. 1627, Seq. ID No. 1633, Seq. ID No. 1645, Seq. ID No. 1647, Seq. ID No. 1649, Seq. ID No. 1655, Seq. ID No. 1665, Seq. ID No. 1667, Seq. ID No. 1671, Seq. ID No. 1677, Seq. ID No. 1679, Seq. ID No. 1681, Seq. ID No. 1695, Seq. ID No. 1703, Seq. ID No. 1705, Seq. ID No. 1731, Seq. ID No. 1753, Seq. ID No. 1759, Seq. ID No. 1763, Seq. ID No. 1787, Seq. ID No. 1797, Seq. ID No. 1821, Seq. ID No. 1823, Seq. ID No. 1833, Seq. ID No. 1839, Seq. ID No. 1841, Seq. ID No. 1843, Seq. ID No. 1893, Seq. ID No. 1917, Seq. ID No. 1919, Seq. ID No. 1971, Seq. ID No. 1991, Seq. ID No. 2013, Seq. ID No. 2015, Seq. ID No. 2017, Seq. ID No. 2019, Seq. ID No. 2033, Seq. ID No. 2035, Seq. ID No. 2049, Seq. ID No. 2081, Seq. ID No. 2083, Seq. ID No. 2085, Seq. ID No. 2111, Seq. ID No. 2113, Seq. ID No. 2123, Seq. ID No. 2125, Seq. ID No. 2127, Seq. ID No. 2131, Seq. ID No. 2133, Seq. ID No. 2153, Seq. ID No. 2161, Seq. ID No. 2171, Seq. ID No. 2175, Seq. ID No. 2179, Seq. ID No. 2187, Seq. ID No. 2201; Seq. ID No. 2203, Seq. ID No. 2221, Seq. ID No. 2237, Seq. ID No. 2249, Seq. ID No. 2251, Seq. ID No. 2255, Seq. ID No. 2261, Seq. ID No. 2263, Seq. ID No. 2271, Seq. ID No. 2279, Seq. ID No. 2283, Seq. ID No. 2285, Seq. ID No. 2289, Seq. ID No. 2295, Seq. ID No. 2305, Seq. ID No. 2307, Seq. ID No. 2317, Seq. ID No. 2319, Seq. ID No. 2323, Seq. ID No. 2325, Seq. ID No. 2343, Seq. ID No. 2349, Seq. ID No. 2385, Seq. ID No. 2393, Seq. ID No. 2423, Seq. ID No. 2453, Seq. ID No. 2491, Seq. ID No. 2497, Seq. ID No. 2499, Seq. ID No. 2507, Seq. ID No. 2515, Seq. ID No. 2517, Seq. ID No. 2521, Seq. ID No. 2523, Seq. ID No. 2527, Seq. ID No. 2533, Seq. ID No. 2535, Seq. ID No. 2541, Seq. ID No. 2543, Seq. ID No. 2547, Seq. ID No. 2549, Seq. ID No. 2557, Seq. ID No. 2573, Seq. ID No. 2583, Seq. ID No. 2601, Seq. ID No. 2603, Seq. ID No. 2621, Seq. ID No. 2653, Seq. ID No. 2681, Seq. ID No. 2691, Seq. ID No. 2701, Seq. ID No. 2733, Seq. ID No. 2739, Seq. ID No. 2801, Seq. ID No. 2807, Seq. ID No. 2815, Seq. ID No. 2819, Seq. ID No. 2821, Seq. ID No. 2823, Seq. ID No. 2833, Seq. ID No. 2837, Seq. ID No. 2839, Seq. ID No. 2845, Seq. ID No. 2853, Seq. ID No. 2861, Seq. ID No. 2865, Seq. ID No. 2873, Seq. ID No. 2875, Seq. ID No. 2877, Seq. ID No. 2883, Seq. ID No. 2895, Seq. ID No. 2905, Seq. ID No. 2939, Seq. ID No. 2941, Seq. ID No. 2947, Seq. ID No. 2981, Seq. ID No. 2983, Seq. ID No. 2987, Seq. ID No. 2991, Seq. ID No. 2995, Seq. ID No. 3001, Seq. ID No. 3005, Seq. ID No. 3021, Seq. ID No. 3025, Seq. ID No. 3043, Seq. ID No. 3045, Seq. ID No. 3073, Seq. ID No. 3075, Seq. ID No. 3155, Seq. ID No. 3163, Seq. ID No. 3165, Seq. ID No. 3167, Seq. ID No. 3183, Seq. ID No. 3213, Seq. ID No. 3215, Seq. ID No. 3235, Seq. ID No. 3245, Seq. ID No. 3261, Seq. ID No. 3263, Seq. ID No. 3265, Seq. ID No. 3269, Seq. ID No. 3271, Seq. ID No. 3275, Seq. ID No. 3277, Seq. ID No. 3317, Seq. ID No. 3325, Seq. ID No. 3337, Seq. ID No. 3351, Seq. ID No. 3389, Seq. ID No. 3405, Seq. ID No. 3419, Seq. ID No. 3421, Seq. ID No. 3423, Seq. ID No. 3451, Seq. ID No. 3453, Seq. ID No. 3455, Seq. ID No. 3491, Seq. ID No. 3495, Seq. ID No. 3503, Seq. ID No. 3505, Seq. ID No. 3517, Seq. ID No. 3519, Seq. ID No. 3545, Seq. ID No. 3547, Seq. ID No. 3571, Seq. ID No. 3593, Seq. ID No. 3617, Seq. ID No. 3621, Seq. ID No. 3629, Seq. ID No. 3663, Seq. ID No. 3701, Seq. ID No. 3703, Seq. ID No. 3723, Seq. ID No. 3737, Seq. ID No. 3741, Seq. ID No. 3745, Seq. ID No. 3747, Seq. ID No. 3759, Seq. ID No. 3763, Seq. ID No. 3767, Seq. ID No. 3769, Seq. ID No. 3771, Seq. ID No. 3785, Seq. ID No. 3793, Seq. ID No. 3813, Seq. ID No. 3837, Seq. ID No. 3839, Seq. ID No. 3861, Seq. ID No. 3865, Seq. ID No. 3889, Seq. ID No. 3891, Seq. ID No. 3895, Seq. ID No. 3915, Seq. ID No. 3923, Seq. ID No. 3925, Seq. ID No. 3929, Seq. ID No. 3965, Seq. ID No. 3975, Seq. ID No. 3979, Seq. ID No. 3995, Seq. ID No. 3997, Seq. ID No. 4009, Seq. ID No. 4039, Seq. ID No. 4041, Seq. ID No. 4061, Seq. ID No. 4079, Seq. ID No. 4081, Seq. ID No. 4111, Seq. ID No. 4113, Seq. ID No. 4135, Seq. ID No. 4139, Seq. ID No. 4155, Seq. ID No. 4181, Seq. ID No. 4203, Seq. ID No. 4221, Seq. ID No. 4247, Seq. ID No. 4253, Seq. ID No. 4257, Seq. ID No. 4259, Seq. ID No. 4261, Seq. ID No. 4263, Seq. ID No. 4269, Seq. ID No. 4311, Seq. ID No. 4379, Seq. ID No. 4389, Seq. ID No. 4391, Seq. ID No. 4471, Seq. ID No. 4473, Seq. ID No. 4481, Seq. ID No. 4525, Seq. ID No. 4551, Seq. ID No. 4561, Seq. ID No. 4573, Seq. ID No. 4617, Seq. ID No. 4639, Seq. ID No. 4659, Seq. ID No. 4681, Seq. ID No. 4701, Seq. ID No. 4705, Seq. ID No. 4713, Seq. ID No. 4717, Seq. ID No. 4721, Seq. ID No. 4737, Seq. ID No. 4741, Seq. ID No. 4813, Seq. ID No. 4817, Seq. ID No. 4819, Seq. ID No. 4829, Seq. ID No. 4831, Seq. ID No. 4851, Seq. ID No. 4869, Seq. ID No. 4875, Seq. ID No. 4913, Seq. ID No. 4919, Seq. ID No. 4929, Seq. ID No. 4931, Seq. ID No. 4983, Seq. ID No. 4993, Seq. ID No. 5011, Seq. ID No. 5023, Seq. ID No. 5031, Seq. ID No. 5033, Seq. ID No. 5035, Seq. ID No. 5043, Seq. ID No. 5061, Seq. ID No. 5063, Seq. ID No. 5069, Seq. ID No. 5103, Seq. ID No. 5121, Seq. ID No. 5123, Seq. ID No. 5131, Seq. ID No. 5133, Seq. ID No. 5135, Seq. ID No. 5137, Seq. ID No. 5139, Seq. ID No. 5141, Seq. ID No. 5151, Seq. ID No. 5159, Seq. ID No. 5161, Seq. ID No. 5165, Seq. ID No. 5167, Seq. ID No. 5175, Seq. ID No. 5177, Seq. ID No. 5205, Seq. ID No. 5207, Seq. ID No. 5211, Seq. ID No. 5215, Seq. ID No. 5219, Seq. ID No. 5223, Seq. ID No. 5285, Seq. ID No. 5287, Seq. ID No. 5329, Seq. ID No. 5331, Seq. ID No. 5377, Seq. ID No. 5379, Seq. ID No. 5381, Seq. ID No. 5385, Seq. ID No. 5405, Seq. ID No. 5407, Seq. ID No. 5453, Seq. ID No. 5459, Seq. ID No. 5461, Seq. ID No. 5463, Seq. ID No. 5481, Seq. ID No. 5509, Seq. ID No. 5529, Seq. ID No. 5531, Seq. ID No. 5557, Seq. ID No. 5569, Seq. ID No. 5577, Seq. ID No. 5623, Seq. ID No. 5625, Seq. ID No. 5643, Seq. ID No. 5659, Seq. ID No. 5661, Seq. ID No. 5683, Seq. ID No. 5685, Seq. ID No. 5709, Seq. ID No. 5711, Seq. ID No. 5733, Seq. ID No. 5739, Seq. ID No. 5765, Seq. ID No. 5797, Seq. ID No. 5827, Seq. ID No. 5889, Seq. ID No. 5893, Seq. ID No. 5905, Seq. ID No. 5909, Seq. ID No. 5911, Seq. ID No. 5923, Seq. ID No. 5963, Seq. ID No. 5965, Seq. ID No. 6025, Seq. ID No. 6027, Seq. ID No. 6031, Seq. ID No. 6043, Seq. ID No. 6045, Seq. ID No. 6047, Seq. ID No. 6053, Seq. ID No. 6059, Seq. ID No. 6109, Seq. ID No. 6111, Seq. ID No. 6115, Seq. ID No. 6117, Seq. ID No. 6127, Seq. ID No. 6131, Seq. ID No. 6135, Seq. ID No. 6137, Seq. ID No. 6161, Seq. ID No. 6165, Seq. ID No. 6183, Seq. ID No. 6189, Seq. ID No. 6191, Seq. ID No. 6193, Seq. ID No. 6207, Seq. ID No. 6209, Seq. ID No. 6217, Seq. ID No. 6225, Seq. ID No. 6235, Seq. ID No. 6243, Seq. ID No. 6249, Seq. ID No. 6253, Seq. ID No. 6259, Seq. ID No. 6265, Seq. ID No. 6267, Seq. ID No. 6275, Seq. ID No. 6283, Seq. ID No. 6285, Seq. ID No. 6303, Seq. ID No. 6325, Seq. ID No. 6327, Seq. ID No. 6363, Seq. ID No. 6365, Seq. ID No. 6367, Seq. ID No. 6385, Seq. ID No. 6425, Seq. ID No. 6427, Seq. ID No. 6429, Seq. ID No. 6431, Seq. ID No. 6453, Seq. ID No. 6465, Seq. ID No. 6469, Seq. ID No. 6471, Seq. ID No. 6479, Seq. ID No. 6539, Seq. ID No. 6541, Seq. ID No. 6571, Seq. ID No. 6573, Seq. ID No. 6639, Seq. ID No. 6643, and Seq. ID No. 6645.

In yet another preferred embodiment, the invention comprises an isolated polynucleotide of the Alloiococcus otitidis genomic sequence encoding a polypeptide, which is predicted by HMM SignaIP algorithm analysis as having a signal peptide, and the polynucleotide is selected from one of Seq. ID No. 3, Seq. ID No. 7, Seq. ID No. 9, Seq. ID No. 19, Seq. ID No. 21, Seq. ID No. 31, Seq. ID No. 33, Seq. ID No. 45, Seq. ID No. 47, Seq. ID No. 49, Seq. ID No. 53, Seq. ID No. 65, Seq. ID No. 67, Seq. ID No. 85, Seq. ID No. 87, Seq. ID No. 105, Seq. ID No. 111, Seq. ID No. 123, Seq. ID No. 131, Seq. ID No. 133, Seq. ID No. 141, Seq. ID No. 159, Seq. ID No. 161, Seq. ID No. 163, Seq. ID No. 171, Seq. ID No. 191, Seq. ID No. 209, Seq. ID No. 211, Seq. ID No. 213, Seq. ID No. 215, Seq. ID No. 217, Seq. ID No. 225, Seq. ID No. 235, Seq. ID No. 237, Seq. ID No. 249, Seq. ID No. 251, Seq. ID No. 255, Seq. ID No. 257, Seq. ID No. 259, Seq. ID No. 271, Seq. ID No. 273, Seq. ID No. 277, Seq. ID No. 285, Seq. ID No. 287, Seq. ID No. 289, Seq. ID No. 327, Seq. ID No. 329, Seq. ID No. 357, Seq. ID No. 359, Seq. ID No. 373, Seq. ID No. 375, Seq. ID No. 393, Seq. ID No. 401, Seq. ID No. 403, Seq. ID No. 455, Seq. ID No. 457, Seq. ID No. 477, Seq. ID No. 487, Seq. ID No. 489, Seq. ID No. 491, Seq. ID No. 493, Seq. ID No. 495, Seq. ID No. 497, Seq. ID No. 499, Seq. ID No. 503, Seq. ID No. 505, Seq. ID No. 509, Seq. ID No. 511, Seq. ID No. 513, Seq. ID No. 515, Seq. ID No. 525, Seq. ID No. 527, Seq. ID No. 545, Seq. ID No. 547, Seq. ID No. 549, Seq. ID No. 557, Seq. ID No. 567, Seq. ID No. 569, Seq. ID No. 575, Seq. ID No. 577, Seq. ID No. 579, Seq. ID No. 591, Seq. ID No. 593, Seq. ID No. 599, Seq. ID No. 617, Seq. ID No. 637, Seq. ID No. 639, Seq. ID No. 641, Seq. ID No. 643, Seq. ID No. 645, Seq. ID No. 647, Seq. ID No. 653, Seq. ID No. 655, Seq. ID No. 657, Seq. ID No. 659, Seq. ID No. 661, Seq. ID No. 667, Seq. ID No. 669, Seq. ID No. 675, Seq. ID No. 677, Seq. ID No. 679, Seq. ID No. 715, Seq. ID No. 717, Seq. ID No. 719, Seq. ID No. 721, Seq. ID No. 723, Seq. ID No. 725, Seq. ID No. 745, Seq. ID No. 747, Seq. ID No. 749, Seq. ID No. 751, Seq. ID No. 755, Seq. ID No. 757, Seq. ID No. 759, Seq. ID No. 761, Seq. ID No. 763, Seq. ID No. 765, Seq. ID No. 769, Seq. ID No. 777, Seq. ID No. 779, Seq. ID No. 781, Seq. ID No. 807, Seq. ID No. 809, Seq. ID No. 819, Seq. ID No. 825, Seq. ID No. 863, Seq. ID No. 893, Seq. ID No. 901, Seq. ID No. 935, Seq. ID No. 937, Seq. ID No. 941, Seq. ID No. 943, Seq. ID No. 955, Seq. ID No. 957, Seq. ID No. 967, Seq. ID No. 979, Seq. ID No. 981, Seq. ID No. 983, Seq. ID No. 985, Seq. ID No. 1001, Seq. ID No. 1003, Seq. ID No. 1005, Seq. ID No. 1007, Seq. ID No. 1009, Seq. ID No. 1011, Seq. ID No. 1013, Seq. ID No. 1015, Seq. ID No. 1019, Seq. ID No. 1029, Seq. ID No. 1031, Seq. ID No. 1033, Seq. ID No. 1035, Seq. ID No. 1037, Seq. ID No. 1039, Seq. ID No. 1045, Seq. ID No. 1055, Seq. ID No. 1075, Seq. ID No. 1077, Seq. ID No. 1105, Seq. ID No. 1107, Seq. ID No. 1131, Seq. ID No. 1135, Seq. ID No. 1137, Seq. ID No. 1139, Seq. ID No. 1151, Seq. ID No. 1165, Seq. ID No. 1167, Seq. ID No. 1169, Seq. ID No. 1173, Seq. ID No. 1183, Seq. ID No. 1195, Seq. ID No. 1197, Seq. ID No. 1203, Seq. ID No. 1213, Seq. ID No. 1223, Seq. ID No. 1227, Seq. ID No. 1229, Seq. ID No. 1237, Seq. ID No. 1243, Seq. ID No. 1245, Seq. ID No. 1259, Seq. ID No. 1265, Seq. ID No. 1267, Seq. ID No. 1269, Seq. ID No. 1271, Seq. ID No. 1283, Seq. ID No. 1287, Seq. ID No. 1289, Seq. ID No. 1291, Seq. ID No. 1295, Seq. ID No. 1297, Seq. ID No. 1303, Seq. ID No. 1305, Seq. ID No. 1315, Seq. ID No. 1317, Seq. ID No. 1319, Seq. ID No. 1325, Seq. ID No. 1327, Seq. ID No. 1329, Seq. ID No. 1331, Seq. ID No. 1337, Seq. ID No. 1341, Seq. ID No. 1343, Seq. ID No. 1347, Seq. ID No. 1349, Seq. ID No. 1351, Seq. ID No. 1353, Seq. ID No. 1365, Seq. ID No. 1367, Seq. ID No. 1369, Seq. ID No. 1371, Seq. ID No. 1381, Seq. ID No. 1401, Seq. ID No. 1429, Seq. ID No. 1435, Seq. ID No. 1445, Seq. ID No. 1447, Seq. ID No. 1481, Seq. ID No. 1513, Seq. ID No. 1515, Seq. ID No. 1517, Seq. ID No. 1529, Seq. ID No. 1533, Seq. ID No. 1535, Seq. ID No. 1545, Seq. ID No. 1547, Seq. ID No. 1551, Seq. ID No. 1553, Seq. ID No. 1559, Seq. ID No. 1561, Seq. ID No. 1563, Seq. ID No. 1571, Seq. ID No. 1579, Seq. ID No. 1583, Seq. ID No. 1585, Seq. ID No. 1621, Seq. ID No. 1633, Seq. ID No. 1639, Seq. ID No. 1643, Seq. ID No. 1645, Seq. ID No. 1647, Seq. ID No. 1649, Seq. ID No. 1653, Seq. ID No. 1661, Seq. ID No. 1663, Seq. ID No. 1665, Seq. ID No. 1671, Seq. ID No. 1673, Seq. ID No. 1677, Seq. ID No. 1679, Seq. ID No. 1681, Seq. ID No. 1693, Seq. ID No. 1707, Seq. ID No. 1711, Seq. ID No. 1731, Seq. ID No. 1733, Seq. ID No. 1737, Seq. ID No. 1753, Seq. ID No. 1755, Seq. ID No. 1757, Seq. ID No. 1765, Seq. ID No. 1767, Seq. ID No. 1771, Seq. ID No. 1781, Seq. ID No. 1787, Seq. ID No. 1791, Seq. ID No. 1797, Seq. ID No. 1805, Seq. ID No. 1819, Seq. ID No. 1821, Seq. ID No. 1823, Seq. ID No. 1839, Seq. ID No. 1857, Seq. ID No. 1869, Seq. ID No. 1875, Seq. ID No. 1891, Seq. ID No. 1893, Seq. ID No. 1895, Seq. ID No. 1915, Seq. ID No. 1917, Seq. ID No. 1919, Seq. ID No. 1923, Seq. ID No. 1927, Seq. ID No. 1929, Seq. ID No. 1969, Seq. ID No. 1981, Seq. ID No. 1991, Seq. ID No. 1993, Seq. ID No. 2005, Seq. ID No. 2013, Seq. ID No. 2017, Seq. ID No. 2019, Seq. ID No. 2025, Seq. ID No. 2027, Seq. ID No. 2033, Seq. ID No. 2035, Seq. ID No. 2041, Seq. ID No. 2049, Seq. ID No. 2053, Seq. ID No. 2055, Seq. ID No. 2087, Seq. ID No. 2089, Seq. ID No. 2091, Seq. ID No. 2105, Seq. ID No. 2107, Seq. ID No. 2109, Seq. ID No. 2113, Seq. ID No. 2115, Seq. ID No. 2121, Seq. ID No. 2123, Seq. ID No. 2125, Seq. ID No. 2127, Seq. ID No. 2149, Seq. ID No. 2151, Seq. ID No. 2155, Seq. ID No. 2157, Seq. ID No. 2159, Seq. ID No. 2171, Seq. ID No. 2173, Seq. ID No. 2177, Seq. ID No. 2187, Seq. ID No. 2191, Seq. ID No. 2205, Seq. ID No. 2237, Seq. ID No. 2239, Seq. ID No. 2245, Seq. ID No. 2249, Seq. ID No. 2251, Seq. ID No. 2255, Seq. ID No. 2259, Seq. ID No. 2271, Seq. ID No. 2283, Seq. ID No. 2289, Seq. ID No. 2291, Seq. ID No. 2293, Seq. ID No. 2295, Seq. ID No. 2303, Seq. ID No. 2305, Seq. ID No. 2307, Seq. ID No. 2317, Seq. ID No. 2319, Seq. ID No. 2321, Seq. ID No. 2323, Seq. ID No. 2325, Seq. ID No. 2327, Seq. ID No. 2329, Seq. ID No. 2333, Seq. ID No. 2343, Seq. ID No. 2347, Seq. ID No. 2349, Seq. ID No. 2351, Seq. ID No. 2353, Seq. ID No. 2355, Seq. ID No. 2357, Seq. ID No. 2365, Seq. ID No. 2367, Seq. ID No. 2371, Seq. ID No. 2375, Seq. ID No. 2387, Seq. ID No. 2389, Seq. ID No. 2391, Seq. ID No. 2393, Seq. ID No. 2407, Seq. ID No. 2419, Seq. ID No. 2421, Seq. ID No. 2425, Seq. ID No. 2427, Seq. ID No. 2429, Seq. ID No. 2433, Seq. ID No. 2449, Seq. ID No. 2459, Seq. ID No. 2487, Seq. ID No. 2489, Seq. ID No. 2491, Seq. ID No. 2493, Seq. ID No. 2497, Seq. ID No. 2499, Seq. ID No. 2501, Seq. ID No. 2503, Seq. ID No. 2507, Seq. ID No. 2515, Seq. ID No. 2517, Seq. ID No. 2523, Seq. ID No. 2525, Seq. ID No. 2527, Seq. ID No. 2531, Seq. ID No. 2533, Seq. ID No. 2535, Seq. ID No. 2537, Seq. ID No. 2541, Seq. ID No. 2543, Seq. ID No. 2545, Seq. ID No. 2555, Seq. ID No. 2557, Seq. ID No. 2565, Seq. ID No. 2569, Seq. ID No. 2571, Seq. ID No. 2583, Seq. ID No. 2591, Seq. ID No. 2597, Seq. ID No. 2599, Seq. ID No. 2601, Seq. ID No. 2607, Seq. ID No. 2611, Seq. ID No. 2621, Seq. ID No. 2627, Seq. ID No. 2629, Seq. ID No. 2651, Seq. ID No. 2679, Seq. ID No. 2681, Seq. ID No. 2683, Seq. ID No. 2691, Seq. ID No. 2693, Seq. ID No. 2695, Seq. ID No. 2697, Seq. ID No. 2699, Seq. ID No. 2701, Seq. ID No. 2703, Seq. ID No. 2705, Seq. ID No. 2707, Seq. ID No. 2717, Seq. ID No. 2729, Seq. ID No. 2731, Seq. ID No. 2733, Seq. ID No. 2783, Seq. ID No. 2785, Seq. ID No. 2801, Seq. ID No. 2809, Seq. ID No. 2813, Seq. ID No. 2815, Seq. ID No. 2819, Seq. ID No. 2821, Seq. ID No. 2823, Seq. ID No. 2831, Seq. ID No. 2833, Seq. ID No. 2835, Seq. ID No. 2837, Seq. ID No. 2839, Seq. ID No. 2841, Seq. ID No. 2845, Seq. ID No. 2853, Seq. ID No. 2855, Seq. ID No. 2859, Seq. ID No. 2861, Seq. ID No. 2873, Seq. ID No. 2881, Seq. ID No. 2893, Seq. ID No. 2905, Seq. ID No. 2935, Seq. ID No. 2937, Seq. ID No. 2939, Seq. ID No. 2941, Seq. ID No. 2947, Seq. ID No. 2949, Seq. ID No. 2951, Seq. ID No. 2969, Seq. ID No. 2973, Seq. ID No. 2975, Seq. ID No. 2977, Seq. ID No. 2981, Seq. ID No. 2983, Seq. ID No. 2987, Seq. ID No. 2991, Seq. ID No. 2995, Seq. ID No. 2997, Seq. ID No. 3005, Seq. ID No. 3007, Seq. ID No. 3009, Seq. ID No. 3011, Seq. ID No. 3013, Seq. ID No. 3023, Seq. ID No. 3025, Seq. ID No. 3027, Seq. ID No. 3029, Seq. ID No. 3031, Seq. ID No. 3043, Seq. ID No. 3055, Seq. ID No. 3063, Seq. ID No. 3071, Seq. ID No. 3073, Seq. ID No. 3075, Seq. ID No. 3077, Seq. ID No. 3119, Seq. ID No. 3121, Seq. ID No. 3123, Seq. ID No. 3145, Seq. ID No. 3155, Seq. ID No. 3157, Seq. ID No. 3163, Seq. ID No. 3165, Seq. ID No. 3173, Seq. ID No. 3183, Seq. ID No. 3193, Seq. ID No. 3201, Seq. ID No. 3213, Seq. ID No. 3215, Seq. ID No. 3217, Seq. ID No. 3219, Seq. ID No. 3241, Seq. ID No. 3243, Seq. ID No. 3245, Seq. ID No. 3249, Seq. ID No. 3255, Seq. ID No. 3257, Seq. ID No. 3261, Seq. ID No. 3263, Seq. ID No. 3269, Seq. ID No. 3275, Seq. ID No. 3277, Seq. ID No. 3297, Seq. ID No. 3317, Seq. ID No. 3323, Seq. ID No. 3325, Seq. ID No. 3331, Seq. ID No. 3335, Seq. ID No. 3337, Seq. ID No. 3339, Seq. ID No. 3349, Seq. ID No. 3351, Seq. ID No. 3353, Seq. ID No. 3355, Seq. ID No. 3357, Seq. ID No. 3359, Seq. ID No. 3375, Seq. ID No. 3377, Seq. ID No. 3389, Seq. ID No. 3391, Seq. ID No. 3395, Seq. ID No. 3405, Seq. ID No. 3415, Seq. ID No. 3421, Seq. ID No. 3425, Seq. ID No. 3427, Seq. ID No. 3433, Seq. ID No. 3441, Seq. ID No. 3449, Seq. ID No. 3451, Seq. ID No. 3453, Seq. ID No. 3455, Seq. ID No. 3457, Seq. ID No. 3463, Seq. ID No. 3481, Seq. ID No. 3491, Seq. ID No. 3493, Seq. ID No. 3503, Seq. ID No. 3505, Seq. ID No. 3517, Seq. ID No. 3519, Seq. ID No. 3527, Seq. ID No. 3531, Seq. ID No. 3539, Seq. ID No. 3561, Seq. ID No. 3573, Seq. ID No. 3581, Seq. ID No. 3587, Seq. ID No. 3593, Seq. ID No. 3595, Seq. ID No. 3611, Seq. ID No. 3617, Seq. ID No. 3619, Seq. ID No. 3621, Seq. ID No. 3625, Seq. ID No. 3629, Seq. ID No. 3635, Seq. ID No. 3677, Seq ID No. 3679, Seq. ID No. 3689, Seq. ID No. 3717, Seq. ID No. 3723, Seq. ID No. 3727, Seq. ID No. 3741, Seq. ID No. 3745, Seq. ID No. 3753, Seq. ID No. 3755, Seq. ID No. 3757, Seq. ID No. 3761, Seq. ID No. 3763, Seq. ID No. 3765, Seq. ID No. 3781, Seq. ID No. 3783, Seq. ID No. 3793, Seq. ID No. 3799, Seq. ID No. 3807, Seq. ID No. 3813, Seq. ID No. 3815, Seq. ID No. 3817, Seq. ID No. 3837, Seq. ID No. 3839, Seq. ID No. 3849, Seq. ID No. 3851, Seq. ID No. 3853, Seq. ID No. 3859, Seq. ID No. 3861, Seq. ID No. 3863, Seq. ID No. 3865, Seq. ID No. 3879, Seq. ID No. 3889, Seq. ID No. 3891, Seq. ID No. 3895, Seq. ID No. 3903, Seq. ID No. 3915, Seq. ID No. 3923, Seq. ID No. 3925, Seq. ID No. 3959, Seq. ID No. 3963, Seq. ID No. 3973, Seq. ID No. 3975, Seq. ID No. 3993, Seq. ID No. 3995, Seq. ID No. 3997, Seq. ID No. 4005, Seq. ID No. 4007, Seq. ID No. 4009, Seq. ID No. 4025, Seq. ID No. 4027, Seq. ID No. 4039, Seq. ID No. 4041, Seq. ID No. 4043, Seq. ID No. 4045, Seq. ID No. 4047, Seq. ID No. 4049, Seq. ID No. 4061, Seq. ID No. 4071, Seq. ID No. 4073, Seq. ID No. 4079, Seq. ID No. 4081, Seq. ID No. 4083, Seq. ID No. 4101, Seq. ID No. 4103, Seq. ID No. 4111, Seq. ID No. 4133, Seq. ID No. 4135, Seq. ID No. 4137, Seq. ID No. 4139, Seq. ID No. 4141, Seq. ID No. 4143, Seq. ID No. 4145, Seq. ID No. 4151, Seq. ID No. 4153, Seq. ID No. 4181, Seq. ID No. 4221, Seq. ID No. 4223, Seq. ID No. 4225, Seq. ID No. 4227, Seq. ID No. 4231, Seq. ID No. 4233, Seq. ID No. 4241, Seq. ID No. 4247, Seq. ID No. 4253, Seq. ID No. 4257, Seq. ID No. 4259, Seq. ID No. 4261, Seq. ID No. 4263, Seq. ID No. 4265, Seq. ID No. 4269, Seq. ID No. 4275, Seq. ID No. 4285, Seq. ID No. 4287, Seq. ID No. 4297, Seq. ID No. 4307, Seq. ID No. 4311, Seq. ID No. 4313, Seq. ID No. 4343, Seq. ID No. 4389, Seq. ID No. 4391, Seq. ID No. 4399, Seq. ID No. 4403, Seq. ID No. 4437, Seq. ID No. 4439, Seq. ID No. 4441, Seq. ID No. 4469, Seq. ID No. 4473, Seq. ID No. 4481, Seq. ID No. 4483, Seq. ID No. 4485, Seq. ID No. 4493, Seq. ID No. 4495, Seq. ID No. 4497, Seq. ID No. 4513, Seq. ID No. 4515, Seq. ID No. 4527, Seq. ID No. 4529, Seq. ID No. 4533, Seq. ID No. 4587, Seq. ID No. 4589, Seq. ID No. 4639, Seq. ID No. 4641, Seq. ID No. 4643, Seq. ID No. 4659, Seq. ID No. 4681, Seq. ID No. 4687, Seq. ID No. 4695, Seq. ID No. 4705, Seq. ID No. 4707, Seq. ID No. 4709, Seq. ID No. 4711, Seq. ID No. 4715, Seq. ID No. 4717, Seq. ID No. 4737, Seq. ID No. 4759, Seq. ID No. 4813, Seq. ID No. 4827, Seq. ID No. 4853, Seq. ID No. 4863, Seq. ID No. 4869, Seq. ID No. 4871, Seq. ID No. 4873, Seq. ID No. 4875, Seq. ID No. 4919, Seq. ID No. 4921, Seq. ID No. 4923, Seq. ID No. 4929, Seq. ID No. 4931, Seq. ID No. 4949, Seq. ID No. 4951, Seq. ID No. 4975, Seq. ID No. 4987, Seq. ID No. 4999, Seq. ID No. 5013, Seq. ID No. 5031, Seq. ID No. 5061, Seq. ID No. 5063, Seq. ID No. 5069, Seq. ID No. 5085, Seq. ID No. 5103, Seq. ID No. 5115, Seq. ID No. 5123, Seq. ID No. 5125, Seq. ID No. 5129, Seq. ID No. 5131, Seq. ID No. 5133, Seq. ID No. 5135, Seq. ID No. 5137, Seq. ID No. 5139, Seq. ID No. 5141, Seq. ID No. 5159, Seq. ID No. 5165, Seq. ID No. 5175, Seq. ID No. 5197, Seq. ID No. 5199, Seq. ID No. 5205, Seq. ID No. 5207, Seq. ID No. 5213, Seq. ID No. 5219, Seq. ID No. 5223, Seq. ID No. 5267, Seq. ID No. 5273, Seq. ID No. 5275, Seq. ID No. 5277, Seq. ID No. 5279, Seq. ID No. 5281, Seq. ID No. 5285, Seq. ID No. 5327, Seq. ID No. 5329, Seq. ID No. 5331, Seq. ID No. 5341, Seq. ID No. 5377, Seq. ID No. 5379, Seq. ID No. 5385, Seq. ID No; 5387, Seq. ID No. 5405, Seq. ID No. 5413, Seq. ID No. 5415, Seq. ID No. 5433, Seq. ID No. 5453, Seq. ID No. 5459, Seq. ID No. 5461, Seq. ID No. 5463, Seq. ID No. 5465, Seq. ID No. 5491, Seq. ID No. 5497, Seq. ID No. 5509, Seq. ID No. 5513, Seq. ID No. 5515, Seq. ID No. 5517, Seq. ID No. 5525, Seq. ID No. 5529, Seq. ID No. 5531, Seq. ID No. 5533, Seq. ID No. 5543, Seq. ID No. 5545, Seq. ID No. 5569, Seq. ID No. 5587, Seq. ID No. 5589, Seq. ID No. 5591, Seq. ID No. 5593, Seq. ID No. 5595, Seq. ID No. 5597, Seq. ID No. 5609, Seq. ID No. 5611, Seq. ID No. 5621, Seq. ID No. 5623, Seq. ID No. 5625, Seq. ID No. 5633, Seq. ID No. 5637, Seq. ID No. 5639, Seq. ID No. 5641, Seq. ID No. 5643, Seq. ID No. 5649, Seq. ID No. 5659, Seq. ID No. 5667, Seq. ID No. 5681, Seq. ID No. 5683, Seq. ID No. 5685, Seq. ID No. 5687, Seq. ID No. 5689, Seq. ID No. 5699, Seq. ID No. 5703, Seq. ID No. 5705, Seq. ID No. 5709, Seq. ID No. 5711, Seq. ID No. 5719, Seq. ID No. 5729, Seq. ID No. 5733, Seq. ID No. 5735, Seq. ID No. 5739, Seq. ID No. 5763, Seq. ID No. 5765, Seq. ID No. 5767, Seq. ID No. 5771, Seq. ID No. 5783, Seq. ID No. 5785, Seq. ID No. 5791, Seq. ID No. 5797, Seq. ID No. 5829, Seq. ID No. 5879, Seq. ID No. 5883, Seq. ID No. 5885, Seq. ID No. 5889, Seq. ID No. 5893, Seq. ID No. 5895, Seq. ID No. 5899, Seq. ID No. 5901, Seq. ID No. 5905, Seq. ID No. 5909, Seq. ID No. 5921, Seq. ID No. 5923, Seq. ID No. 5927, Seq. ID No. 5929, Seq. ID No. 5939, Seq. ID No. 6023, Seq. ID No. 6025, Seq. ID No. 6027, Seq. ID No. 6043, Seq. ID No. 6047, Seq. ID No. 6053, Seq. ID No. 6059, Seq. ID No. 6063, Seq. ID No. 6067, Seq. ID No. 6087, Seq. ID No. 6089, Seq. ID No. 6091, Seq. ID No. 6095, Seq. ID No. 6103, Seq. ID No. 6107, Seq. ID No. 6109, Seq. ID No. 6111, Seq. ID No. 6115, Seq. ID No. 6117, Seq. ID No. 6135, Seq. ID No. 6137, Seq. ID No. 6159, Seq. ID No. 6161, Seq. ID No. 6163, Seq. ID No. 6165, Seq. ID No. 6169, Seq. ID No. 6183, Seq. ID No. 6185, Seq. ID No. 6187, Seq. ID No. 6189, Seq. ID No. 6191, Seq. ID No. 6197, Seq. ID No. 6203, Seq. ID No. 6205, Seq. ID No. 6209, Seq. ID No. 6217, Seq. ID No. 6221, Seq. ID No. 6223, Seq. ID No. 6225, Seq. ID No. 6227, Seq. ID No. 6229, Seq. ID No. 6235, Seq. ID No. 6237, Seq. ID No. 6241, Seq. ID No. 6243, Seq. ID No. 6245, Seq. ID No. 6249, Seq. ID No. 6253, Seq. ID No. 6259, Seq. ID No. 6263, Seq. ID No. 6265, Seq. ID No. 6267, Seq. ID No. 6273, Seq. ID No. 6275, Seq. ID No. 6283, Seq. ID No. 6285, Seq. ID No. 6289, Seq. ID No. 6301, Seq. ID No. 6303, Seq. ID No. 6325, Seq. ID No. 6327, Seq. ID No. 6363, Seq. ID No. 6365, Seq. ID No. 6377, Seq. ID No. 6381, Seq. ID No. 6383, Seq. ID No. 6415, Seq. ID No. 6425, Seq. ID No. 6427, Seq. ID No. 6429, Seq. ID No. 6431, Seq. ID No. 6473, Seq. ID No. 6479, Seq. ID No. 6497, Seq. ID No. 6517, Seq. ID No. 6541, Seq. ID No. 6543, Seq. ID No. 6555, Seq. ID No. 6557, Seq. ID No. 6571, Seq. ID No. 6573, Seq. ID No. 6581, Seq. ID No. 6593, Seq. ID No. 6595, Seq. ID No. 6597, Seq. ID No. 6599, Seq. ID No. 6601, Seq. ID No. 6603, Seq. ID No. 6631, Seq. ID No. 6637, Seq. ID No. 6639, Seq. ID No. 6643, Seq. ID No. 6645, and Seq. ID No. 6649.

In still yet another preferred embodiment, the invention comprises an isolated polynucleotide of the Alloiococcus otitidis genomic sequence encoding a polypeptide, which is predicted by HMM SignaIP algorithm analysis as being a non-secretory protein, and the polypeptide is selected from Seq. ID No. 1, Seq. ID No. 5, Seq. ID No. 11, Seq.

ID No. 13, Seq. ID No. 15, Seq. ID No. 17, Seq. ID No. 23, Seq. ID No. 25, Seq. ID No. 27, Seq. ID No. 29, Seq. ID No. 35, Seq. ID No. 37, Seq. ID No. 39, Seq. ID No. 41, Seq. ID No. 43, Seq. ID No. 51, Seq. ID No. 55, Seq. ID No. 57, Seq. ID No. 59, Seq. ID No. 61, Seq. ID No. 63, Seq. ID No. 69, Seq. ID No. 71, Seq. ID No. 73, Seq. ID No. 75, Seq. ID No. 77, Seq. ID No. 79, Seq. ID No. 81, Seq. ID No. 83, Seq. ID No. 89, Seq. ID No. 91, Seq. ID No. 93, Seq. ID No. 95, Seq. ID No. 97, Seq. ID No. 99, Seq. ID No. 101, Seq. ID No. 103, Seq. ID No. 107, Seq. ID No. 109, Seq. ID No. 113, Seq. ID No. 115, Seq. ID No. 117, Seq. ID No. 119, Seq. ID No. 121, Seq. ID No. 125, Seq. ID No. 127, Seq. ID No. 129, Seq. ID No. 135, Seq. ID No. 137, Seq. ID No. 139, Seq. ID No. 143, Seq. ID No. 145, Seq. ID No. 147, Seq. ID No. 149, Seq. ID No. 151, Seq. ID No. 153, Seq. ID No. 155, Seq. ID No. 157, Seq. ID No. 165, Seq. ID No. 167, Seq. ID No. 169, Seq. ID No. 173, Seq. ID No. 175, Seq. ID No. 177, Seq. ID No. 179, Seq. ID No. 181, Seq. ID No. 183, Seq. ID No. 185, Seq. ID No. 187, Seq. ID No. 189, Seq. ID No. 193, Seq. ID No. 195, Seq. ID No. 197, Seq. ID No. 199, Seq. ID No. 201, Seq. ID No. 203, Seq. ID No. 205, Seq. ID No. 207, Seq. ID No. 219, Seq. ID No. 221, Seq. ID No. 223, Seq. ID No. 227, Seq. ID No. 229, Seq. ID No. 231, Seq. ID No. 233, Seq. ID No. 239, Seq. ID No. 241, Seq. ID No. 243, Seq. ID No. 245, Seq. ID No. 247, Seq. ID No. 253, Seq. ID No. 261, Seq. ID No. 263, Seq. ID No. 265, Seq. ID No. 267, Seq. ID No. 269, Seq. ID No. 275, Seq. ID No. 279, Seq. ID No. 281, Seq. ID No. 283, Seq. ID No. 291, Seq. ID No. 293, Seq. ID No. 295, Seq. ID No. 297, Seq. ID No. 299, Seq. ID No. 301, Seq. ID No. 303, Seq. ID No. 305, Seq. ID No. 307, Seq. ID No. 309, Seq. ID No. 311, Seq. ID No. 313, Seq. ID No. 315, Seq. ID No. 317, Seq. ID No. 319, Seq. ID No. 321, Seq. ID No. 323, Seq. ID No. 325, Seq. ID No. 331, Seq. ID No. 333, Seq. ID No. 335, Seq. ID No. 337, Seq. ID No. 339, Seq. ID No. 341, Seq. ID No. 343, Seq. ID No. 345, Seq. ID No. 347, Seq. ID No. 349, Seq. ID No. 351, Seq. ID No. 353, Seq. ID No. 355, Seq. ID No. 361, Seq. ID No. 363, Seq. ID No. 365, Seq. ID No. 367, Seq. ID No. 369, Seq. ID No. 371, Seq. ID No. 377, Seq. ID No. 379, Seq. ID No. 381, Seq. ID No. 383, Seq. ID No. 385, Seq. ID No. 387, Seq. ID No. 389, Seq. ID No. 391, Seq. ID No. 395, Seq. ID No. 397, Seq. ID No. 399, Seq. ID No. 405, Seq. ID No. 407, Seq. ID No. 409, Seq. ID No. 411, Seq. ID No. 413, Seq. ID No. 415, Seq. ID No. 417, Seq. ID No. 419, Seq. ID No. 421, Seq. ID No. 423, Seq. ID No. 425, Seq. ID No. 427, Seq. ID No. 429, Seq. ID No. 431, Seq. ID No. 433, Seq. ID No. 435, Seq. ID No. 437, Seq. ID No. 439, Seq. ID No. 441, Seq. ID No. 443, Seq. ID No. 445, Seq. ID No. 447, Seq. ID No. 449, Seq. ID No. 451, Seq. ID No. 453, Seq. ID No. 459, Seq. ID No. 461, Seq. ID No. 463, Seq. ID No. 465, Seq. ID No. 467, Seq. ID No. 469, Seq. ID No. 471, Seq. ID No. 473, Seq. ID No. 475, Seq. ID No. 479, Seq. ID No. 481, Seq. ID No. 483, Seq. ID No. 485, Seq. ID No. 501, Seq. ID No. 507, Seq. ID No. 517, Seq. ID No. 519, Seq. ID No. 521, Seq. ID No. 523, Seq. ID No. 529, Seq. ID No. 531, Seq. ID No. 533, Seq. ID No. 535, Seq. ID No. 637, Seq. ID No. 539, Seq. ID No. 541, Seq. ID No. 543, Seq. ID No. 551, Seq. ID No. 553, Seq. ID No. 555, Seq. ID No. 559, Seq. ID No. 561, Seq. ID No. 563, Seq. ID No. 565, Seq. ID No. 571, Seq. ID No. 573, Seq. ID No. 581, Seq. ID No. 583, Seq. ID No. 585, Seq. ID No. 587, Seq. ID No. 589, Seq. ID No. 595, Seq. ID No. 597, Seq. ID No. 601, Seq. ID No. 603, Seq. ID No. 605, Seq. ID No. 607, Seq. ID No. 609, Seq. ID No. 611, Seq. ID No. 613, Seq. ID No. 615, Seq. ID No. 619, Seq. ID No. 621, Seq. ID No. 623, Seq. ID No. 625, Seq. ID No. 627, Seq. ID No. 629, Seq. ID No. 631, Seq. ID No. 633, Seq. ID No. 635, Seq. ID No. 649, Seq. ID No. 651, Seq. ID No. 663, Seq. ID No. 665, Seq. ID No. 671, Seq. ID No. 673, Seq. ID No. 681, Seq. ID No. 683, Seq. ID No. 685, Seq. ID No. 687, Seq. ID No. 689, Seq. ID No. 691, Seq. ID No. 693, Seq. ID No. 695, Seq. ID No. 697, Seq. ID No. 699, Seq. ID No. 701, Seq. ID No. 703, Seq. ID No. 705, Seq. ID No. 707, Seq. ID No. 709, Seq. ID No. 711, Seq. ID No. 713, Seq. ID No. 727, Seq. ID No. 729, Seq. ID No. 731, Seq. ID No. 733, Seq. ID No. 735, Seq. ID No. 737, Seq. ID No. 739, Seq. ID No. 741, Seq. ID No. 743, Seq. ID No. 753, Seq. ID No. 767, Seq. ID No. 771, Seq. ID No. 773, Seq. ID No. 775, Seq. ID No. 783, Seq. ID No. 785, Seq. ID No. 787, Seq. ID No. 789, Seq. ID No. 791, Seq. ID No. 793, Seq. ID No. 795, Seq. ID No. 797, Seq. ID No. 799, Seq. ID No. 801, Seq. ID No. 803, Seq. ID No. 805, Seq. ID No. 811, Seq. ID No. 813, Seq. ID No. 815, Seq. ID No. 817, Seq. ID No. 821, Seq. ID No. 823, Seq. ID No. 827, Seq. ID No. 829, Seq. ID No. 831, Seq. ID No. 833, Seq. ID No. 835, Seq. ID No. 837, Seq. ID No. 839, Seq. ID No. 841, Seq. ID No. 843, Seq. ID No. 845, Seq. ID No. 847, Seq. ID No. 849, Seq. ID No. 851, Seq. ID No. 853, Seq. ID No. 855, Seq. ID No. 857, Seq. ID No. 859, Seq. ID No. 861, Seq. ID No. 865, Seq. ID No. 867, Seq. ID No. 869, Seq. ID No. 871, Seq. ID No. 873, Seq. ID No. 875, Seq. ID No. 877, Seq. ID No. 879, Seq. ID No. 881, Seq. ID No. 883, Seq. ID No. 885, Seq. ID No. 887, Seq. ID No. 889, Seq. ID No. 891, Seq. ID No. 895, Seq. ID No. 897, Seq. ID No. 899, Seq. ID No. 903, Seq. ID No. 905, Seq. ID No. 907, Seq. ID No. 909, Seq. ID No. 911, Seq. ID No. 913, Seq. ID No. 915, Seq. ID No. 917, Seq. ID No. 919, Seq. ID No. 921, Seq. ID No. 923, Seq. ID No. 925, Seq. ID No. 927, Seq. ID No. 929, Seq. ID No. 931, Seq. ID No. 933, Seq. ID No. 939, Seq. ID No. 945, Seq. ID No. 947, Seq. ID No. 949, Seq. ID No. 951, Seq. ID No. 953, Seq. ID No. 959, Seq. ID No. 961, Seq. ID No. 963, Seq. ID No. 965, Seq. ID No. 969, Seq. ID No. 971, Seq. ID No. 973, Seq. ID No. 975, Seq. ID No. 977, Seq. ID No. 987, Seq. ID No. 989, Seq. ID No. 991, Seq. ID No. 993, Seq. ID No. 995, Seq. ID No. 997, Seq. ID No. 999, Seq. ID No. 1017, Seq. ID No. 1021, Seq. ID No. 1023, Seq. ID No. 1025, Seq. ID No. 1027, Seq. ID No. 1041, Seq. ID No. 1043, Seq. ID No. 1047, Seq. ID No. 1049, Seq. ID No. 1051, Seq. ID No. 1053, Seq. ID No. 1057, Seq. ID No. 1059, Seq. ID No. 1061, Seq. ID No. 1063, Seq. ID No. 1065, Seq. ID No. 1067, Seq. ID No. 1069, Seq. ID No. 1071, Seq. ID No. 1073, Seq. ID No. 1079, Seq. ID No. 1081, Seq. ID No. 1083, Seq. ID No. 1085, Seq. ID No. 1087, Seq. ID No. 1089, Seq. ID No. 1091, Seq. ID No. 1093, Seq. ID No. 1095, Seq. ID No. 1097, Seq. ID No. 1099, Seq. ID No. 1101, Seq. ID No. 1103, Seq. ID No. 1109, Seq. ID No. 1111, Seq. ID No. 1113, Seq. ID No. 1115, Seq. ID No. 1117, Seq. ID No. 1119, Seq. ID No. 1121, Seq. ID No. 1123, Seq. ID No. 1125, Seq. ID No. 1127, Seq. ID No. 1129, Seq. ID No. 1133, Seq. ID No. 1141, Seq. ID No. 1143, Seq. ID No. 1145, Seq. ID No. 1147, Seq. ID No. 1149, Seq. ID No. 1153, Seq. ID No. 1155, Seq. ID No. 1157, Seq. ID No. 1159, Seq. ID No. 1161, Seq. ID No. 1163, Seq. ID No. 1171, Seq. ID No. 1175, Seq. ID No. 1177, Seq. ID No. 1179, Seq. ID No. 1181, Seq. ID No. 1185, Seq. ID No. 1187, Seq. ID No. 1189, Seq. ID No. 1191, Seq. ID No. 1193, Seq. ID No. 1199, Seq. ID No. 1201, Seq. ID No. 1205, Seq. ID No. 1207, Seq. ID No. 1209, Seq. ID No. 1211, Seq. ID No. 1215, Seq. ID No. 1217, Seq. ID No. 1219, Seq. ID No. 1221, Seq. ID No. 1225, Seq. ID No. 1231, Seq. ID No. 1233, Seq. ID No. 1235, Seq. ID No. 1239, Seq. ID No. 1241, Seq. ID No. 1247, Seq. ID No. 1249, Seq. ID No. 1251, Seq. ID No. 1253, Seq. ID No. 1255, Seq. ID No. 1257, Seq. ID No. 1261, Seq. ID No. 1263, Seq. ID No. 1273, Seq. ID No. 1275, Seq. ID No. 1277, Seq. ID No. 1279, Seq. ID No. 1281, Seq. ID No. 1285, Seq. ID No. 1293, Seq. ID No. 1299, Seq. ID No. 1301, Seq. ID No. 1307, Seq. ID No. 1309, Seq. ID No. 1311, Seq. ID No. 1313, Seq. ID No. 1321, Seq. ID No. 1323, Seq. ID No. 1333, Seq. ID No. 1335, Seq. ID No. 1339, Seq. ID No. 1345, Seq. ID No. 1355, Seq. ID No. 1357, Seq. ID No. 1359, Seq. ID No. 1361, Seq. ID No. 1363, Seq. ID No. 1373, Seq. ID No. 1375, Seq. ID No. 1377, Seq. ID No. 1379, Seq. ID No. 1383, Seq. ID No. 1385, Seq. ID No. 1387, Seq. ID No. 1389, Seq. ID No. 1391, Seq. ID No. 1393, Seq. ID No. 1395, Seq. ID No. 1397, Seq. ID No. 1399, Seq. ID No. 1403, Seq. ID No. 1405, Seq. ID No. 1407, Seq. ID No. 1409, Seq. ID No. 1411, Seq. ID No. 1413, Seq. ID No. 1415, Seq. ID No. 1417, Seq. ID No. 1419, Seq. ID No. 1421, Seq. ID No. 1423, Seq. ID No. 1425, Seq. ID No. 1427, Seq. ID No. 1431, Seq. ID No. 1433, Seq. ID No. 1437, Seq. ID No. 1439, Seq. ID No. 1441, Seq. ID No. 1443, Seq. ID No. 1449, Seq. ID No. 1451, Seq. ID No. 1453, Seq. ID No. 1455, Seq. ID No. 1457, Seq. ID No. 1459, Seq. ID No. 1461, Seq. ID No. 1463, Seq. ID No. 1465, Seq. ID No. 1467, Seq. ID No. 1469, Seq. ID No. 1471, Seq. ID No. 1473, Seq. ID No. 1475, Seq. ID No. 1477, Seq. ID No. 1479, Seq. ID No. 1483, Seq. ID No. 1485, Seq. ID No. 1487, Seq. ID No. 1489, Seq. ID No. 1491, Seq. ID No. 1493, Seq. ID No. 1495, Seq. ID No. 1497, Seq. ID No. 1499, Seq. ID No. 1501, Seq. ID No. 1503, Seq. ID No. 1505, Seq. ID No. 1507, Seq. ID No. 1509, Seq. ID No. 1511, Seq. ID No. 1519, Seq. ID No. 1521, Seq. ID No. 1523, Seq. ID No. 1525, Seq. ID No. 1527, Seq. ID No. 1531, Seq. ID No. 1537, Seq. ID No. 1539, Seq. ID No. 1541, Seq. ID No. 1543, Seq. ID No. 1549, Seq. ID No. 1555, Seq. ID No. 1557, Seq. ID No. 1565, Seq. ID No. 1567, Seq. ID No. 1569, Seq. ID No. 1573, Seq. ID No. 1575, Seq. ID No. 1577, Seq. ID No. 1581, Seq. ID No. 1587, Seq. ID No. 1589, Seq. ID No. 1591, Seq. ID No. 1593, Seq. ID No. 1595, Seq. ID No. 1597, Seq. ID No. 1601, Seq. ID No. 1603, Seq. ID No. 1605, Seq. ID No. 1607, Seq. ID No. 1609, Seq. ID No. 1611, Seq. ID No. 1613, Seq. ID No. 1615, Seq. ID No. 1617, Seq. ID No. 1619, Seq. ID No. 1623, Seq. ID No. 1625, Seq. ID No. 1627, Seq. ID No. 1629, Seq. ID No. 1631, Seq. ID No. 1635, Seq. ID No. 1637, Seq. ID No. 1641, Seq. ID No. 1651, Seq. ID No. 1655, Seq. ID No. 1657, Seq. ID No. 1659, Seq. ID No. 1667, Seq. ID No. 1669, Seq. ID No. 1675, Seq. ID No. 1683, Seq. ID No. 1685, Seq. ID No. 1687, Seq. ID No. 1689, Seq. ID No. 1691, Seq. ID No. 1695, Seq. ID No. 1697, Seq. ID No. 1699, Seq. ID No. 1701, Seq. ID No. 1703, Seq. ID No. 1705, Seq. ID No. 1709, Seq. ID No. 1713, Seq. ID No. 1715, Seq. ID No. 1717, Seq. ID No. 1719, Seq. ID No. 1721, Seq. ID No. 1723, Seq. ID No. 1725, Seq. ID No. 1727, Seq. ID No. 1729, Seq. ID No. 1735, Seq. ID No. 1739, Seq. ID No. 1741, Seq. ID No. 1743, Seq. ID No. 1745, Seq. ID No. 1747, Seq. ID No. 1749, Seq. ID No. 1751, Seq. ID No. 1759, Seq. ID No. 1761, Seq. ID No. 1763, Seq. ID No. 1769, Seq. ID No. 1773, Seq. ID No. 1775, Seq. ID No. 1777, Seq. ID No. 1779, Seq. ID No. 1783, Seq. ID No. 1785, Seq. ID No. 1789, Seq. ID No. 1793, Seq. ID No. 1795, Seq. ID No. 1799, Seq. ID No. 1801, Seq. ID No. 1803, Seq. ID No. 1807, Seq. ID No. 1809, Seq. ID No. 1811, Seq. ID No. 1813, Seq. ID No. 1815, Seq. ID No. 1817, Seq. ID No. 1825, Seq. ID No. 1827, Seq. ID No. 1829, Seq. ID No. 1831, Seq. ID No. 1833, Seq. ID No. 1835, Seq. ID No. 1837, Seq. ID No. 1841, Seq. ID No. 1843, Seq. ID No. 1845, Seq. ID No. 1847, Seq. ID No. 1849, Seq. ID No. 1851, Seq. ID No. 1853, Seq. ID No. 1855, Seq. ID No. 1859, Seq. ID No. 1861, Seq. ID No. 1863, Seq. ID No. 1865, Seq. ID No. 1867, Seq. ID No. 1871, Seq. ID No. 1873, Seq. ID No. 1877, Seq. ID No. 1879, Seq. ID No. 1881, Seq. ID No. 1883, Seq. ID No. 1885, Seq. ID No. 1887, Seq. ID No. 1889, Seq. ID No. 1897, Seq. ID No. 1899, Seq. ID No. 1901, Seq. ID No. 1903, Seq. ID No. 1905, Seq. ID No. 1907, Seq. ID No. 1909, Seq. ID No. 1911, Seq. ID No. 1913, Seq. ID No. 1921, Seq. ID No. 1925, Seq. ID No. 1931, Seq. ID No. 1933, Seq. ID No. 1935, Seq. ID No. 1937, Seq. ID No. 1939, Seq. ID No. 1941, Seq. ID No. 1943, Seq. ID No. 1945, Seq. ID No. 1947, Seq. ID No. 1949, Seq. ID No. 1951, Seq. ID No. 1953, Seq. ID No. 1955, Seq. ID No. 1957, Seq. ID No. 1959, Seq. ID No. 1961, Seq. ID No. 1963, Seq. ID No. 1965, Seq. ID No. 1967, Seq. ID No. 1971, Seq. ID No. 1973, Seq. ID No. 1975, Seq. ID No. 1977, Seq. ID No. 1979, Seq. ID No. 1983, Seq. ID No. 1985, Seq. ID No. 1987, Seq. ID No. 1989, Seq. ID No. 1995, Seq. ID No. 1997, Seq. ID No. 1999, Seq. ID No. 2001, Seq. ID No. 2003, Seq. ID No. 2007, Seq. ID No. 2009, Seq. ID No. 2011, Seq. ID No. 2015, Seq. ID No. 2021, Seq. ID No. 2023, Seq. ID No. 2029, Seq. ID No. 2031, Seq. ID No. 2037, Seq. ID No. 2039, Seq. ID No. 2043, Seq. ID No. 2045, Seq. ID No. 2047, Seq. ID No. 2051, Seq. ID No. 2057, Seq. ID No. 2059, Seq. ID No. 2061, Seq. ID No. 2063, Seq. ID No. 2065, Seq. ID No. 2067, Seq. ID No. 2069, Seq. ID No. 2071, Seq. ID No. 2073, Seq. ID No. 2075, Seq. ID No. 2077, Seq. ID No. 2079, Seq. ID No. 2081, Seq. ID No. 2083, Seq. ID No. 2085, Seq. ID No. 2093, Seq. ID No. 2095, Seq. ID No. 2097, Seq. ID No. 2099, Seq. ID No. 2101, Seq. ID No. 2103, Seq. ID No. 2111, Seq. ID No. 2117, Seq. ID No. 2119, Seq. ID No. 2129, Seq. ID No. 2131, Seq. ID No. 2133, Seq. ID No. 2135, Seq. ID No. 2137, Seq. ID No. 2139, Seq. ID No. 2141, Seq. ID No. 2143, Seq. ID No. 2145, Seq. ID No. 2147, Seq. ID No. 2153, Seq. ID No. 2161, Seq. ID No. 2163, Seq. ID No. 2165, Seq. ID No. 2167, Seq. ID No. 2169, Seq. ID No. 2175, Seq. ID No. 2179, Seq. ID No. 2181, Seq. ID No. 2183, Seq. ID No. 2185, Seq. ID No. 2189, Seq. ID No. 2193, Seq. ID No. 2195, Seq. ID No. 2197, Seq. ID No. 2199, Seq. ID No. 2201, Seq. ID No. 2203, Seq. ID No. 2207, Seq. ID No. 2209, Seq. ID No. 2211, Seq. ID No. 2213, Seq. ID No. 2215, Seq. ID No. 2217, Seq. ID No. 2219, Seq. ID No. 2221, Seq. ID No. 2223, Seq. ID No. 2225, Seq. ID No. 2227, Seq. ID No. 2229, Seq. ID No. 2231, Seq. ID No. 2233, Seq. ID No. 2235, Seq. ID No. 2241, Seq. ID No. 2243, Seq. ID No. 2247, Seq. ID No. 2253, Seq. ID No. 2257, Seq. ID No. 2261, Seq. ID No. 2263, Seq. ID No. 2265, Seq. ID No. 2267, Seq. ID No. 2269, Seq. ID No. 2273, Seq. ID No. 2275, Seq. ID No. 2277, Seq. ID No. 2279, Seq. ID No. 2281, Seq. ID No. 2285, Seq. ID No. 2287, Seq. ID No. 2297, Seq. ID No. 2299, Seq. ID No. 2301, Seq. ID No. 2309, Seq. ID No. 2311, Seq. ID No. 2313, Seq. ID No. 2315, Seq. ID No. 2331, Seq. ID No. 2335, Seq. ID No. 2337, Seq. ID No. 2339, Seq. ID No. 2341, Seq. ID No. 2345, Seq. ID No. 2359, Seq. ID No. 2361, Seq. ID No. 2363, Seq. ID No. 2369, Seq. ID No. 2373, Seq. ID No. 2377, Seq. ID No. 2379, Seq. ID No. 2381, Seq. ID No. 2383, Seq. ID No. 2385, Seq. ID No. 2395, Seq. ID No. 2397, Seq. ID No. 2399, Seq. ID No. 2401, Seq. ID No. 2403, Seq. ID No. 2405, Seq. ID No. 2409, Seq. ID No. 2411, Seq. ID No. 2413, Seq. ID No. 2415, Seq. ID No. 2417, Seq. ID No. 2423, Seq. ID No. 2431, Seq. ID No. 2435, Seq. ID No. 2437, Seq. ID No. 2439, Seq. ID No. 2441, Seq. ID No. 2443, Seq. ID No. 2445, Seq. ID No. 2447, Seq. ID No. 2451, Seq. ID No. 2453, Seq. ID No. 2455, Seq. ID No. 2457, Seq. ID No. 2461, Seq. ID No. 2463, Seq. ID No. 2465, Seq. ID No. 2467, Seq. ID No. 2469, Seq. ID No. 2471, Seq. ID No. 2473, Seq. ID No. 2475, Seq. ID No. 2477, Seq. ID No. 2479, Seq. ID No. 2481, Seq. ID No. 2483, Seq. ID No. 2485, Seq. ID No. 2495, Seq. ID No. 2505, Seq. ID No. 2509, Seq. ID No. 2511, Seq. ID No. 2513, Seq. ID No. 2519, Seq. ID No. 2521, Seq. ID No. 2529, Seq. ID No. 2539, Seq. ID No. 2547, Seq. ID No. 2549, Seq. ID No. 2551, Seq. ID No. 2553, Seq. ID No. 2559, Seq. ID No. 2561, Seq. ID No. 2563, Seq. ID No. 2567, Seq. ID No. 2573, Seq. ID No. 2575, Seq. ID No. 2577, Seq. ID No. 2579, Seq. ID No. 2581, Seq. ID No. 2585, Seq. ID No. 2587, Seq. ID No. 2589, Seq. ID No. 2593, Seq. ID No. 2595, Seq. ID No. 2603, Seq. ID No. 2605, Seq. ID No. 2609, Seq. ID No. 2613, Seq. ID No. 2615, Seq. ID No. 2617, Seq. ID No. 2619, Seq. ID No. 2623, Seq. ID No. 2625, Seq. ID No. 2631, Seq. ID No. 2633, Seq. ID No. 2635, Seq. ID No. 2637, Seq. ID No. 2639, Seq. ID No. 2641, Seq. ID No. 2643, Seq. ID No. 2645, Seq. ID No. 2647, Seq. ID No. 2649, Seq. ID No. 2653, Seq. ID No. 2655, Seq. ID No. 2657, Seq. ID No. 2659, Seq. ID No. 2661, Seq. ID No. 2663, Seq. ID No. 2665, Seq. ID No. 2667, Seq. ID No. 2669, Seq. ID No. 2671, Seq. ID No. 2673, Seq. ID No. 2675, Seq. ID No. 2677, Seq. ID No. 2685, Seq. ID No. 2687, Seq. ID No. 2689, Seq. ID No. 2709, Seq. ID No. 2711, Seq. ID No. 2713, Seq. ID No. 2715, Seq. ID No. 2719, Seq. ID No. 2721, Seq. ID No. 2723, Seq. ID No. 2725, Seq. ID No. 2727, Seq. ID No. 2735, Seq. ID No. 2737, Seq. ID No. 2739, Seq. ID No. 2741, Seq. ID No. 2743, Seq. ID No. 2745, Seq. ID No. 2747, Seq. ID No. 2749, Seq. ID No. 2751, Seq. ID No. 2753, Seq. ID No. 2755,;Seq. ID No. 2757, Seq. ID No. 2759, Seq. ID No. 2761, Seq. ID No. 2763, Seq. ID No. 2765, Seq. ID No. 2767, Seq. ID No. 2769, Seq. ID No. 2771, Seq. ID No. 2773, Seq. ID No. 2775, Seq. ID No. 2777, Seq. ID No. 2779, Seq. ID No. 2781, Seq. ID No. 2787, Seq. ID No. 2789, Seq. ID No. 2791, Seq. ID No. 2793, Seq. ID No. 2795, Seq. ID No. 2797, Seq. ID No. 2799, Seq. ID No. 2803, Seq. ID No. 2805, Seq. ID No. 2807, Seq. ID No. 2811, Seq. ID No. 2817, Seq. ID No. 2825, Seq. ID No. 2827, Seq. ID No. 2829, Seq. ID No. 2843, Seq. ID No. 2847, Seq. ID No. 2849, Seq. ID No. 2851, Seq. ID No. 2857, Seq. ID No. 2863, Seq. ID No. 2865, Seq. ID No. 2867, Seq. ID No. 2869, Seq. ID No. 2871, Seq. ID No. 2875, Seq. ID No. 2877, Seq. ID No. 2879, Seq. ID No. 2883, Seq. ID No. 2885, Seq. ID No. 2887, Seq. ID No. 2889, Seq. ID No. 2891, Seq. ID No. 2895, Seq. ID No. 2897, Seq. ID No. 2899, Seq. ID No. 2901, Seq. ID No. 2903, Seq. ID No. 2907, Seq. ID No. 2909, Seq. ID No. 2911, Seq. ID No. 2913, Seq. ID No. 2915, Seq. ID No. 2917, Seq. ID No. 2919, Seq. ID No. 2921, Seq. ID No. 2923, Seq. ID No. 2925, Seq. ID No. 2927, Seq. ID No. 2929, Seq. ID No. 2931, Seq. ID No. 2933, Seq. ID No. 2943, Seq. ID No. 2945, Seq. ID No. 2953, Seq. ID No. 2955, Seq. ID No. 2957, Seq. ID No. 2959, Seq. ID No. 2961, Seq. ID No. 2963, Seq. ID No. 2965, Seq. ID No. 2967, Seq. ID No. 2971, Seq. ID No. 2979, Seq. ID No. 2985, Seq. ID No. 2989, Seq. ID No. 2993, Seq. ID No. 2999, Seq. ID No. 3001, Seq. ID No. 3003, Seq. ID No. 3015, Seq. ID No. 3017, Seq. ID No. 3019, Seq. ID No. 3021, Seq. ID No. 3033, Seq. ID No. 3035, Seq. ID No. 3037, Seq. ID No. 3039, Seq. ID No. 3041, Seq. ID No. 3045, Seq. ID No. 3047, Seq. ID No. 3049, Seq. ID No. 3051, Seq. ID No. 3053, Seq. ID No. 3057, Seq. ID No. 3059, Seq. ID No. 3061, Seq. ID No. 3065, Seq. ID No. 3067, Seq. ID No. 3069, Seq. ID No. 3079, Seq. ID No. 3081, Seq. ID No. 3083, Seq. ID No. 3085, Seq. ID No. 3087, Seq. ID No. 3089, Seq. ID No. 3091, Seq. ID No. 3093, Seq. ID No. 3095, Seq. ID No. 3097, Seq. ID No. 3099, Seq. ID No. 3101, Seq. ID No. 3103, Seq. ID No. 3105, Seq. ID No. 3107, Seq. ID No. 3109, Seq. ID No. 3111, Seq. ID No. 3113, Seq. ID No. 3115, Seq. ID No. 3117, Seq. ID No. 3125, Seq. ID No. 3127, Seq. ID No. 3129, Seq. ID No. 3131, Seq. ID No. 3133, Seq. ID No. 3135, Seq. ID No. 3137, Seq. ID No. 3139, Seq. ID No. 3141, Seq. ID No. 3143, Seq. ID No. 3147, Seq. ID No. 3149, Seq. ID No. 3151, Seq. ID No. 3153, Seq. ID No. 3159, Seq. ID No. 3161, Seq. ID No. 3167, Seq. ID No. 3169, Seq. ID No. 3171, Seq. ID No. 3175, Seq. ID No. 3177, Seq. ID No. 3179, Seq. ID No. 3181, Seq. ID No. 3185, Seq. ID No. 3187, Seq. ID No. 3189, Seq. ID No. 3191, Seq. ID No. 3195, Seq. ID No. 3197, Seq. ID No. 3199, Seq. ID No. 3203, Seq. ID No. 3205, Seq. ID No. 3207, Seq. ID No. 3209, Seq. ID No. 3211, Seq. ID No. 3221, Seq. ID No. 3223, Seq. ID No. 3225, Seq. ID No. 3227, Seq. ID No. 3229, Seq. ID No. 3231, Seq. ID No. 3233, Seq. ID No. 3235, Seq. ID No. 3237, Seq. ID No. 3239, Seq. ID No. 3247, Seq. ID No. 3251, Seq. ID No. 3253, Seq. ID No. 3259, Seq. ID No. 3265, Seq. ID No. 3267, Seq. ID No. 3271, Seq. ID No. 3273, Seq. ID No. 3279, Seq. ID No. 3281, Seq. ID No. 3283, Seq. ID No. 3285, Seq. ID No. 3287, Seq. ID No. 3289, Seq. ID No. 3291, Seq. ID No. 3293, Seq. ID No. 3295, Seq. ID No. 3299, Seq. ID No. 3301, Seq. ID No. 3303, Seq. ID No. 3305, Seq. ID No. 3307, Seq. ID No. 3309, Seq. ID No. 3311, Seq. ID No. 3313, Seq. ID No. 3315, Seq. ID No. 3319, Seq. ID No. 3321, Seq. ID No. 3327, Seq. ID No. 3329, Seq. ID No. 3333, Seq. ID No. 3341, Seq. ID No. 3343, Seq. ID No. 3345, Seq. ID No. 3347, Seq. ID No. 3361, Seq. ID No. 3363, Seq. ID No. 3365, Seq. ID No. 3367, Seq. ID No. 3369, Seq. ID No. 3371, Seq. ID No. 3373, Seq. ID No. 3379, Seq. ID No. 3381, Seq. ID No. 3383, Seq. ID No. 3385, Seq. ID No. 3387, Seq. ID No. 3393, Seq. ID No. 3397, Seq. ID No. 3399, Seq. ID No. 3401, Seq. ID No. 3403, Seq. ID No. 3407, Seq. ID No. 3409, Seq. ID No. 3411, Seq. ID No. 3413, Seq. ID No. 3417, Seq. ID No. 3419, Seq. ID No. 3423, Seq. ID No. 3429, Seq. ID No. 3431, Seq. ID No. 3435, Seq. ID No. 3437, Seq. ID No. 3439, Seq. ID No. 3443, Seq. ID No. 3445, Seq. ID No. 3447, Seq. ID No. 3459, Seq. ID No. 3461, Seq. ID No. 3465, Seq. ID No. 3467, Seq. ID No. 3469, Seq. ID No. 3471, Seq. ID No. 3473, Seq. ID No. 3475, Seq. ID No. 3477, Seq. ID No. 3479, Seq. ID No. 3483, Seq. ID No. 3485, Seq. ID No. 3487, Seq. ID No. 3489, Seq. ID No. 3495, Seq. ID No. 3497, Seq. ID No. 3499, Seq. ID No. 3501, Seq. ID No. 3507, Seq. ID No. 3509, Seq. ID No. 3511, Seq. ID No. 3513, Seq. ID No. 3515, Seq. ID No. 3521, Seq. ID No. 3523, Seq. ID No. 3525, Seq. ID No. 3529, Seq. ID No. 3533, Seq. ID No. 3535, Seq. ID No. 3537, Seq. ID No. 3541, Seq. ID No. 3543, Seq. ID No. 3545, Seq. ID No. 3547, Seq. ID No. 3549, Seq. ID No. 3551, Seq. ID No. 3553, Seq. ID No. 3555, Seq. ID No. 3557, Seq. ID No. 3559, Seq. ID No. 3563, Seq. ID No. 3565, Seq. ID No. 3567, Seq. ID No. 3569, Seq. ID No. 3571, Seq. ID No. 3575, Seq. ID No. 3577, Seq. ID No. 3579, Seq. ID No. 3583, Seq. ID No. 3585, Seq. ID No. 3589, Seq. ID No. 3591, Seq. ID No. 3597, Seq. ID No. 3599, Seq. ID No. 3601, Seq. ID No. 3603, Seq. ID No. 3605, Seq. ID No. 3607, Seq. ID No. 3609, Seq. ID No. 3613, Seq. ID No. 3615, Seq. ID No. 3623, Seq. ID No. 3627, Seq. ID No. 3631, Seq. ID No. 3633, Seq. ID No. 3637, Seq. ID No. 3639, Seq. ID No. 3641, Seq. ID No. 3643, Seq. ID No. 3645, Seq. ID No. 3647, Seq. ID No. 3649, Seq. ID No. 3651, Seq. ID No. 3653, Seq. ID No. 3655, Seq. ID No. 3657, Seq. ID No. 3659, Seq. ID No. 3661, Seq. ID No. 3663, Seq. ID No. 3665, Seq. ID No. 3667, Seq. ID No. 3669, Seq. ID No. 3671, Seq. ID No. 3673, Seq. ID No. 3675, Seq. ID No. 3681, Seq. ID No. 3683, Seq. ID No. 3685, Seq. ID No. 3687, Seq. ID No. 3691, Seq. ID No. 3693, Seq. ID No. 3695, Seq. ID No. 3697, Seq. ID No. 3699, Seq. ID No. 3701, Seq. ID No. 3703, Seq. ID No. 3705, Seq. ID No. 3707, Seq. ID No. 3709, Seq. ID No. 3711, Seq. ID No. 3713, Seq. ID No. 3715, Seq. ID No. 3719, Seq. ID No. 3721, Seq. ID No. 3725, Seq. ID No. 3729, Seq. ID No. 3731, Seq. ID No. 3733, Seq. ID No. 3735, Seq. ID No. 3737, Seq. ID No. 3739, Seq. ID No. 3743, Seq. ID No. 3747, Seq. ID No. 3749, Seq. ID No. 3751, Seq. ID No. 3759, Seq. ID No. 3767, Seq. ID No. 3769, Seq. ID No. 3771, Seq. ID No. 3773, Seq. ID No. 3775, Seq. ID No. 3777, Seq. ID No. 3779, Seq. ID No. 3785, Seq. ID No. 3787, Seq. ID No. 3789, Seq. ID No. 3791, Seq. ID No. 3795, Seq. ID No. 3797, Seq. ID No. 3801, Seq. ID No. 3803, Seq. ID No. 3805, Seq. ID No. 3809, Seq. ID No. 3811, Seq. ID No. 3819, Seq. ID No. 3821, Seq. ID No. 3823, Seq. ID No. 3825, Seq. ID No. 3827, Seq. ID No. 3829, Seq. ID No. 3831, Seq. ID No. 3833, Seq. ID No. 3835, Seq. ID No. 3841, Seq. ID No. 3843, Seq. ID No. 3845, Seq. ID No. 3847, Seq. ID No. 3855, Seq. ID No. 3857, Seq. ID No. 3867, Seq. ID No. 3869, Seq. ID No. 3871, Seq. ID No. 3873, Seq. ID No. 3875, Seq. ID No. 3877, Seq. ID No. 3881, Seq. ID No. 3883, Seq. ID No. 3885, Seq. ID No. 3887, Seq. ID No. 3893, Seq. ID No. 3897, Seq. ID No. 3899, Seq. ID No. 3901, Seq. ID No. 3905, Seq. ID No. 3907, Seq. ID No. 3909, Seq. ID No. 3911, Seq. ID No. 3913, Seq. ID No. 3917, Seq. ID No. 3919, Seq. ID No. 3921, Seq. ID No. 3927, Seq. ID No. 3929, Seq. ID No. 3931, Seq. ID No. 3933, Seq. ID No. 3935, Seq. ID No. 3937, Seq. ID No. 3939, Seq. ID No. 3941, Seq. ID No. 3943, Seq. ID No. 3945, Seq. ID No. 3947, Seq. ID No. 3949, Seq. ID No. 3951, Seq. ID No. 3953, Seq. ID No. 3955, Seq. ID No. 3957, Seq. ID No. 3961, Seq. ID No. 3965, Seq. ID No. 3967, Seq. ID No. 3969, Seq. ID No. 3971, Seq. ID No. 3977, Seq. ID No. 3979, Seq. ID No. 3981, Seq. ID No. 3983, Seq. ID No. 3985, Seq. ID No. 3987, Seq. ID No. 3989, Seq. ID No. 3991, Seq. ID No. 3999, Seq. ID No. 4001, Seq. ID No. 4003, Seq. ID No. 4011, Seq. ID No. 4013, Seq. ID No. 4015, Seq. ID No. 4017, Seq. ID No. 4019, Seq. ID No. 4021, Seq. ID No. 4023, Seq. ID No. 4029, Seq. ID No. 4031, Seq. ID No. 4033, Seq. ID No. 4035, Seq. ID No. 4037, Seq. ID No. 4051, Seq. ID No. 4053, Seq. ID No. 4055, Seq. ID No. 4057, Seq. ID No. 4059, Seq. ID No. 4063, Seq. ID No. 4065, Seq. ID No. 4067, Seq. ID No. 4069, Seq. ID No. 4075, Seq. ID No. 4077, Seq. ID No. 4085, Seq. ID No. 4087, Seq. ID No. 4089, Seq. ID No. 4091, Seq. ID No. 4093, Seq. ID No. 4095, Seq. ID No. 4097, Seq. ID No. 4099, Seq. ID No. 4105, Seq. ID No. 4107, Seq. ID No. 4109, Seq. ID No. 4113, Seq. ID No. 4115, Seq. ID No. 4117, Seq. ID No. 4119, Seq. ID No. 4121, Seq. ID No. 4123, Seq. ID No. 4125, Seq. ID No. 4127, Seq. ID No. 4129, Seq. ID No. 4131, Seq. ID No. 4147, Seq. ID No. 4149, Seq. ID No. 4155, Seq. ID No. 4157, Seq. ID No. 4159, Seq. ID No. 4161, Seq. ID No. 4163, Seq. ID No. 4165, Seq. ID No. 4167, Seq. ID No. 4169, Seq. ID No. 4171, Seq. ID No. 4173, Seq. ID No. 4175, Seq. ID No. 4177, Seq. ID No. 4179, Seq. ID No. 4183, Seq. ID No. 4185, Seq. ID No. 4187, Seq. ID No. 4189, Seq. ID No. 4191, Seq. ID No. 4193, Seq. ID No. 4195, Seq. ID No. 4197, Seq. ID No. 4199, Seq. ID No. 4201, Seq. ID No. 4203, Seq. ID No. 4205, Seq. ID No. 4207, Seq. ID No. 4209, Seq. ID No. 4211, Seq. ID No. 4213, Seq. ID No. 4215, Seq. ID No. 4217, Seq. ID No. 4219, Seq. ID No. 4229, Seq. ID No. 4235, Seq. ID No. 4237, Seq. ID No. 4239, Seq. ID No. 4243, Seq. ID No. 4245, Seq. ID No. 4249, Seq. ID No. 4251, Seq. ID No. 4255, Seq. ID No. 4267, Seq. ID No. 4271, Seq. ID No. 4273, Seq. ID No. 4277, Seq. ID No. 4279, Seq. ID No. 4281,.Seq. ID No. 4283, Seq. ID No. 4289, Seq. ID No. 4291, Seq. ID No. 4293, Seq. ID No. 4295, Seq. ID No. 4299, Seq. ID No. 4301, Seq. ID No. 4303, Seq. ID No. 4305, Seq. ID No. 4309, Seq. ID No. 4315, Seq. ID No. 4317, Seq. ID No. 4319, Seq. ID No. 4321, Seq. ID No. 4323, Seq. ID No. 4325, Seq. ID No. 4327, Seq. ID No. 4329, Seq. ID No. 4331, Seq. ID No. 4333, Seq. ID No. 4335, Seq. ID No. 4337, Seq. ID No. 4339, Seq. ID No. 4341, Seq. ID No. 4345, Seq. ID No. 4347, Seq. ID No. 4349, Seq. ID No. 4351, Seq. ID No. 4353, Seq. ID No. 4355, Seq. ID No. 4357, Seq. ID No. 4359, Seq. ID No. 4361, Seq. ID No. 4363, Seq. ID No. 4365, Seq. ID No. 4367, Seq. ID No. 4369, Seq. ID No. 4371, Seq. ID No. 4373, Seq. ID No. 4375, Seq. ID No. 4377, Seq. ID No. 4381, Seq. ID No. 4383, Seq. ID No. 4387, Seq. ID No. 4393, Seq. ID No. 4395, Seq. ID No. 4397, Seq. ID No. 4401, Seq. ID No. 4405, Seq. ID No. 4407, Seq. ID No. 4409, Seq. ID No. 4411, Seq. ID No. 4413, Seq. ID No. 4415, Seq. ID No. 4417, Seq. ID No. 4419, Seq. ID No. 4421, Seq. ID No. 4423, Seq. ID No. 4425, Seq. ID No. 4427, Seq. ID No. 4429, Seq. ID No. 4431, Seq. ID No. 4433, Seq. ID No. 4435, Seq. ID No. 4443, Seq. ID No. 4445, Seq. ID No. 4447, Seq. ID No. 4449, Seq. ID No. 4451, Seq. ID No. 4453, Seq. ID No. 4455, Seq. ID No. 4457, Seq. ID No. 4459, Seq. ID No. 4461, Seq. ID No. 4463, Seq. ID No. 4465, Seq. ID No. 4467, Seq. ID No. 4471, Seq. ID No. 4475, Seq. ID No. 4477, Seq. ID No. 4479, Seq. ID No. 4487, Seq. ID No. 4489, Seq. ID No. 4491, Seq. ID No. 4499, Seq. ID No. 4501, Seq. ID No. 4503, Seq. ID No. 4505, Seq. ID No. 4507, Seq. ID No. 4509, Seq. ID No. 4511, Seq. ID No. 4517, Seq. ID No. 4519, Seq. ID No. 4521, Seq. ID No. 4523, Seq. ID No. 4525, Seq. ID No. 4531, Seq. ID No. 4535, Seq. ID No. 4537, Seq. ID No. 4539, Seq. ID No. 4541, Seq. ID No. 4543, Seq. ID No. 4545, Seq. ID No. 4547, Seq. ID No. 4549, Seq. ID No. 4551, Seq. ID No. 4553, Seq. ID No. 4555, Seq. ID No. 4557, Seq. ID No. 4559, Seq. ID No. 4561, Seq. ID No. 4563, Seq. ID No. 4565, Seq. ID No. 5 4567, Seq. ID No. 4569, Seq. ID No. 4571, Seq. ID No. 4573, Seq. ID No. 4575, Seq. ID No. 4577, Seq. ID No. 4579, Seq. ID No. 4581, Seq. ID No. 4583, Seq. ID No. 4585, Seq. ID No. 4591, Seq. ID No. 4593, Seq. ID No. 4595, Seq. ID No. 4597, Seq. ID No. 4599, Seq. ID No. 4601, Seq. ID No. 4603, Seq. ID No. 4605, Seq. ID No. 4607, Seq. ID No. 4609, Seq. ID No. 4611, Seq. ID No. 4613, Seq. ID No. 4615, Seq. ID No. 4617, lo Seq. ID No. 4619, Seq. ID No. 4621, Seq. ID No. 4623, Seq. ID No. 4625, Seq. ID No. 4627, Seq. ID No. 4629, Seq. ID No. 4631, Seq. ID No. 4633, Seq. ID No. 4635, Seq. ID No. 4637, Seq. ID No. 4645, Seq. ID No. 4647, Seq. ID No. 4649, Seq. ID No. 4651, Seq. ID No. 4653, Seq. ID No. 4655, Seq. ID No. 4657, Seq. ID No. 4661, Seq. ID No. 4663, Seq. ID No. 4665, Seq. ID No. 4667, Seq. ID No. 4669, Seq. ID No. 4671, Seq. ID 15 No. 4673, Seq. ID No. 4675, Seq. ID No. 4677, Seq. ID No. 4679, Seq. ID No. 4683, Seq. ID No. 4685, Seq. ID No. 4689, Seq. ID No. 4691, Seq. ID No. 4693, Seq. ID No. 4697, Seq. ID No. 4699, Seq. ID No. 4701, Seq. ID No. 4703, Seq. ID No. 4713, Seq. ID No. 4719, Seq. ID No. 4721, Seq. ID No. 4723, Seq. ID No. 4725, Seq. ID No. 4727, Seq. ID No. 4729, Seq. ID No. 4731, Seq. ID No. 4733, Seq. ID No. 4735, Seq. ID No. 20 4739, Seq. ID No. 4741, Seq. ID No. 4743, Seq. ID No. 4745, Seq. ID No. 4747, Seq. ID No. 4749, Seq. ID No. 4751, Seq. ID No. 4753, Seq. ID No. 4755, Seq. ID No. 4757, Seq. ID No. 4761, Seq. ID No. 4763, Seq. ID No. 4765, Seq. ID No. 4767, Seq. ID No. 4769, Seq. ID No. 4771, Seq. ID No. 4773, Seq. ID No. 4775, Seq. ID No. 4777, Seq. ID No. 4779, Seq. ID No. 4781, Seq. ID No. 4783, Seq. ID No. 4785, Seq. ID No. 4787, 25 Seq. ID No. 4789, Seq. ID No. 4791, Seq. ID No. 4793, Seq. ID No. 4795, Seq. ID No. 4797, Seq. ID No. 4799, Seq. ID No. 4801, Seq. ID No. 4803, Seq. ID No. 4805, Seq. ID No. 4807, Seq. ID No. 4809, Seq. ID No. 4811, Seq. ID No. 4815, Seq. ID No. 4817, Seq. ID No. 4819, Seq. ID No. 4821, Seq. ID No. 4823, Seq. ID No. 4825, Seq. ID No. 4829, Seq. ID No. 4831, Seq. ID No. 4833, Seq. ID No. 4835, Seq. ID No. 4837, Seq. ID 30 No. 4839, Seq. ID No. 4841, Seq. ID No. 4843, Seq. ID No. 4845, Seq. ID No. 4847, Seq. ID No. 4849, Seq. ID No. 4851, Seq. ID No. 4855, Seq. ID No. 4857, Seq. ID No. 4859, Seq. ID No. 4861, Seq. ID No. 4865, Seq. ID No. 4867, Seq. ID No. 4877, Seq. ID No. 4879, Seq. ID No. 4881, Seq. ID No. 4883, Seq. ID No. 4885, Seq. ID No. 4887, Seq. ID No. 4889, Seq. ID No. 4891, Seq. ID No. 4893, Seq. ID No. 4895, Seq. ID No. 4897, Seq. ID No. 4899, Seq. ID No. 4901, Seq. ID No. 4903, Seq. ID No. 4905, Seq. ID No. 4907, Seq. ID No. 4909, Seq. ID No. 4911, Seq. ID No. 4913, Seq. ID No. 4915, Seq. ID No. 4917, Seq. ID No. 4925, Seq. ID No. 4927, Seq. ID No. 4933, Seq. ID No. 4935, Seq. ID No. 4937, Seq. ID No. 4939, Seq. ID No. 4941, Seq. ID No. 4943, Seq. ID No. 4945, Seq. ID No. 4947, Seq. ID No. 4953, Seq. ID No. 4955, Seq. ID No. 4957, Seq. ID No. 4959, Seq. ID No. 4961, Seq. ID No. 4963, Seq. ID No. 4965, Seq. ID No. 4967, Seq. ID No. 4969, Seq. ID No. 4971, Seq. ID No. 4973, Seq. ID No. 4977, Seq. ID No. 4979, Seq. ID No. 4981, Seq. ID No. 4983, Seq. ID No. 4985, Seq. ID No. 4989, Seq. ID No. 4991, Seq. ID No. 4993, Seq. ID No. 4995, Seq. ID No. 4997, Seq. ID No. 5001, Seq. ID No. 5003, Seq. ID No. 5005, Seq. ID No. 5007, Seq. ID No. 5009, Seq. ID No. 5011, Seq. ID No. 5015, Seq. ID No. 5017, Seq. ID No. 5019, Seq. ID No. 5021, Seq. ID No. 5023, Seq. ID No. 5025, Seq. ID No. 5027, Seq. ID No. 5029, Seq. ID No. 5033, Seq. ID No. 5035, Seq. ID No. 5037, Seq. ID No. 5039, Seq. ID No. 5041, Seq. ID No. 5043, Seq. ID No. 5045, Seq. ID No. 5047, Seq. ID No. 5049, Seq. ID No. 5051, Seq. ID No. 5053, Seq. ID No. 5055, Seq. ID No. 5057, Seq. ID No. 5059, Seq. ID No. 5065, Seq. ID No. 5067; Seq. ID No. 5071, Seq. ID No. 5073, Seq. ID No. 5075, Seq. ID No. 5077, Seq. ID No. 5079, Seq. ID No. 5081, Seq. ID No. 5083, Seq. ID No. 5087, Seq. ID No. 5089, Seq. ID No. 5091, Seq. ID No. 5093, Seq. ID No. 5095, Seq. ID No. 5097, Seq. ID No. 5099, Seq. ID No. 5101, Seq. ID No. 5105, Seq. ID No. 5107, Seq. ID No. 5109, Seq. ID No. 5111, Seq. ID No. 5113, Seq. ID No. 5117, Seq. ID No. 5119, Seq. ID No. 5121, Seq. ID No. 5127, Seq. ID No. 5143, Seq. ID No. 5145, Seq. ID No. 5147, Seq. ID No. 5149, Seq. ID No. 5151, Seq. ID No. 5153, Seq. ID No. 5155, Seq. ID No. 5157, Seq. ID No. 5161, Seq. ID No. 5163, Seq. ID No. 5167, Seq. ID No. 5169, Seq. ID No. 5171, Seq. ID No. 5173, Seq. ID No. 5177, Seq. ID No. 5179, Seq. ID No. 5181, Seq. ID No. 5183, Seq. ID No. 5185, Seq. ID No. 5187, Seq. ID No. 5189, Seq. ID No. 5191, Seq. ID No. 5193, Seq. ID No. 5195, Seq. ID No. 5201, Seq. ID No. 5203, Seq. ID No. 5209, Seq. ID No. 5211, Seq. ID No. 5215, Seq. ID No. 5217, Seq. ID No. 5221, Seq. ID No. 5225, Seq. ID No. 5227, Seq. ID No. 5229, Seq. ID No. 5231, Seq. ID No. 5233, Seq. ID No. 5235, Seq. ID No. 5237, Seq. ID No. 5239, Seq. ID No. 5241, Seq. ID No. 5243, Seq. ID No. 5245, Seq. ID No. 5247, Seq. ID No. 5249, Seq. ID No. 5251, Seq. ID No. 5253, Seq. ID No. 5255, Seq. ID No. 5257, Seq. ID No. 5259, Seq. ID No. 5261, Seq. ID No. 5263, Seq. ID No. 5265, Seq. ID No. 5269, Seq. ID No. 5271, Seq. ID No. 5283, Seq. ID No. 5287, Seq. ID No. 5289, Seq. ID No. 5291, Seq. ID No. 5293, Seq. ID No. 5295, Seq. ID No. 5297, Seq. ID No. 5299, Seq. ID No. 5301, Seq. ID No. 5303, Seq. ID No. 5305, Seq. ID No. 5307, Seq. ID No. 5309, Seq. ID No. 5311, Seq. ID No. 5313, Seq. ID No. 5315, Seq. ID No. 5317, Seq. ID No. 5319, Seq. ID No. 5321, Seq. ID No. 5323, Seq. ID No. 5325, Seq. ID No. 5333, Seq. ID No. 5335, Seq. ID No. 5337, Seq. ID No. 5339, Seq. ID No. 5343, Seq. ID No. 5345, Seq. ID No. 5347, Seq. ID No. 5349, Seq. ID No. 5351, Seq. ID No. 5353, Seq. ID No. 5355, Seq. ID No. 5357, Seq. ID No. 5359, Seq. ID No. 5361, Seq. ID No. 5363, Seq. ID No. 5365, Seq. ID No. 5367, Seq. ID No. 5369, Seq. ID No. 5371, Seq. ID No. 5373, Seq. ID No. 5375, Seq. ID No. 5381, Seq. ID No. 5383, Seq. ID No. 5389, Seq. ID No. 5391, Seq. ID No. 5393, Seq. ID No. 5395, Seq. ID No. 5397, Seq. ID No. 5399, Seq. ID No. 5401, Seq. ID No. 5403, Seq. ID No. 5407, Seq. ID No. 5409, Seq. ID No. 5411, Seq. ID No. 5417, Seq. ID No. 5419, Seq. ID No. 5421, Seq. ID No. 5423, Seq. ID No. 5425, Seq. ID No. 5427, Seq. ID No. 5429, Seq. ID No. 5431, Seq. ID No. 5435, Seq. ID No. 5437, Seq. ID No. 5439, Seq. ID No. 5441, Seq. ID No. 5443, Seq. ID No. 5445, Seq. ID No. 5447, Seq. ID No. 5449, Seq. ID No. 5451, Seq. ID No. 5455, Seq. ID No. 5457, Seq. ID No. 5467, Seq. ID No. 5469, Seq. ID No. 5471, Seq. ID No. 5473, Seq. ID No. 5475, Seq. ID No. 5477, Seq. ID No. 5479, Seq. ID No. 5481, Seq. ID No. 5483, Seq. ID No. 5485, Seq. ID No. 5487, Seq. ID No. 5489, Seq. ID No. 5493, Seq. ID No. 5495, Seq. ID No. 5499, Seq. ID No. 5501, Seq. ID No. 5503, Seq. ID No. 5505, Seq. ID No. 5507, Seq. ID No. 5511, Seq. ID No. 5519, Seq. ID No. 5521, Seq. ID No. 5523, Seq. ID No. 5527, Seq. ID No. 5535, Seq. ID No. 5537, Seq. ID No. 5539, Seq. ID No. 5541, Seq. ID No. 5547, Seq. ID No. 5549, Seq. ID No. 5551, Seq. ID No. 5553, Seq. ID No. 5555, Seq. ID No. 5557, Seq. ID No. 5559, Seq. ID No. 5561, Seq. ID No. 5563, Seq. ID No. 5565, Seq. ID No. 5567, Seq. ID No. 5571, Seq. ID No. 5573, Seq. ID No. 5575, Seq. ID No. 5577, Seq. ID No. 5579, Seq. ID No. 5581, Seq. ID No. 5583, Seq. ID No. 5585, Seq. ID No. 5599, Seq. ID No. 5601, Seq. ID No. 5603, Seq. ID No. 5605, Seq. ID No. 5607, Seq. ID No. 5613, Seq. ID No. 5615, Seq. ID No. 5617, Seq. ID No. 5619, Seq. ID No. 5627, Seq. ID No. 5629, Seq. ID No. 5631, Seq. ID No. 5635, Seq. ID No. 5645, Seq. ID No. 5647, Seq. ID No. 5651, Seq. ID No. 5653, Seq. ID No. 5655, Seq. ID No. 5657, Seq. ID No. 5661, Seq. ID No. 5663, Seq. ID No. 5665, Seq. ID No. 5669, Seq. ID No. 5671, Seq. ID No. 5673, Seq. ID No. 5675, Seq. ID No. 5677, Seq. ID No. 5679, Seq. ID No. 5691, Seq. ID No. 5693, Seq. ID No. 5695, Seq. ID No. 5697, Seq. ID No. 5701, Seq. ID No. 5707, Seq. ID No. 5713, Seq. ID No. 5715, Seq. ID No. 5717, Seq. ID No. 5721, Seq. ID No. 5723, Seq. ID No. 5725, Seq. ID No. 5727, Seq. ID No. 5731, Seq. ID No. 5737, Seq. ID No. 5741, Seq. ID No. 5743, Seq. ID No. 5745, Seq. ID No. 5747, Seq. ID No. 5749, Seq. ID No. 5751, Seq. ID No. 5753, Seq. ID No. 5755, Seq. ID No. 5757, Seq. ID No. 5759, Seq. ID No. 5761, Seq. ID No. 5769, Seq. ID No. 5773, Seq. ID No. 5775, Seq. ID No. 5777, Seq. ID No. 5779, Seq. ID No. 5781, Seq. ID No. 5787, Seq. ID No. 5789, Seq. ID No. 5793, Seq. ID No. 5795, Seq. ID No. 5799, Seq. ID No. 5801, Seq. ID No. 5803, Seq. ID No. 5805, Seq. ID No. 5807, Seq. ID No. 5809, Seq. ID No. 5811, Seq. ID No. 5813, Seq. ID No. 5815, Seq. ID No. 5817, Seq. ID No. 5819, Seq. ID No. 5821, Seq. ID No. 5823, Seq. ID No. 5825, Seq. ID No. 5827, Seq. ID No. 5831, Seq. ID No. 5833, Seq. ID No. 5835, Seq. ID No. 5837, Seq. ID No. 5839, Seq. ID No. 5841, Seq. ID No. 5843, Seq. ID No. 5845, Seq. ID No. 5847, Seq. ID No. 5849, Seq. ID No. 5851, Seq. ID No. 5853, Seq. ID No. 5855, Seq. ID No. 5857, Seq. ID No. 5859, Seq. ID No. 5861, Seq. ID No. 5863, Seq. ID No. 5865, Seq. ID No. 5867, Seq. ID No. 5869, Seq. ID No. 5871, Seq. ID No. 5873, Seq. ID No. 5875, Seq. ID No. 5877, Seq. ID No. 5881, Seq. ID No. 5887, Seq. ID No. 5891, Seq. ID No. 5897, Seq. ID No. 5903, Seq. ID No. 5907, Seq. ID No. 5911, Seq. ID No. 5913, Seq. ID No. 5915, Seq. ID No. 5917, Seq. ID No. 5919, Seq. ID No. 5925, Seq. ID No. 5931, Seq. ID No. 5933, Seq. ID No. 5935, Seq. ID No. 5937, Seq. ID No. 5941, Seq. ID No. 5943, Seq. ID No. 5945, Seq. ID No. 5947, Seq. ID No. 5949, Seq. ID No. 5951, Seq. ID No. 5953, Seq. ID No. 5955, Seq. ID No. 5957, Seq. ID No. 5959, Seq. ID No. 5961, Seq. ID No. 5963, Seq. ID No. 5965, Seq. ID No. 5967, Seq. ID No. 5969, Seq. ID No. 5971, Seq. ID No. 5973, Seq. ID No. 5975, Seq. ID No. 5977, Seq. ID No. 5979, Seq. ID No. 5981, Seq. ID No. 5983, Seq. ID No. 5985, Seq. ID No. 5987, Seq. ID No. 5989, Seq. ID No. 5991, Seq. ID No. 5993, Seq. ID No. 5995, Seq. ID No. 5997, Seq. ID No. 5999, Seq. ID No. 6001, Seq. ID No. 6003, Seq. ID No. 6005, Seq. ID No. 6007, Seq. ID No. 6009, Seq. ID No. 6011, Seq. ID No. 6013, Seq. ID No. 6015, Seq. ID No. 6017, Seq. ID No. 6019, Seq. ID No. 6021, Seq. ID No. 6029, Seq. ID No. 6031, Seq. ID No. 6033, Seq. ID No. 6035, Seq. ID No. 6037, Seq. ID No. 6039, Seq. ID No. 6041, Seq. ID No. 6045, Seq. ID No. 6049, Seq. ID No. 6051, Seq. ID No. 6055, Seq. ID No. 6057, Seq. ID No. 6061, Seq. ID No. 6065, Seq. ID No. 6069, Seq. ID No. 6071, Seq. ID No. 6073, Seq. ID No. 6075, Seq. ID No. 6077, Seq. ID No. 6079, Seq. ID No. 6081, Seq. ID No. 6083, Seq. ID No. 6085, Seq. ID No. 6093, Seq. ID No. 6097, Seq. ID No. 6099, Seq. ID No. 6101, Seq. ID No. 6105, Seq. ID No. 6113, Seq. ID No. 6119, Seq. ID No. 6121, Seq. ID No. 6123, Seq. ID No. 6125, Seq. ID No. 6127, Seq. ID No. 6129, Seq. ID No. 6131, Seq. ID No. 6133, Seq. ID No. 6139, Seq. ID No. 6141, Seq. ID No. 6143, Seq. ID No. 6145, Seq. ID No. 6147, Seq. ID No. 6149, Seq. ID No. 6151, Seq. ID No. 6153, Seq. ID No. 6155, Seq. ID No. 6157, Seq. ID No. 6167, Seq. ID No. 6171, Seq. ID No. 6173, Seq. ID No. 6175, Seq. ID No. 6177, Seq. ID No. 6179, Seq. ID No. 6181, Seq. ID No. 6193, Seq. ID No. 6195, Seq. ID No. 6199, Seq. ID No. 6201, Seq. ID No. 6207, Seq. ID No. 6211, Seq. ID No. 6213, Seq. ID No. 6215, Seq. ID No. 6219, Seq. ID No. 6231, Seq. ID No. 6233, Seq. ID No. 6239, Seq. ID No. 6247, Seq. ID No. 6251, Seq. ID No. 6255, Seq. ID No. 6257, Seq. ID No. 6261, Seq. ID No. 6269, Seq. ID No. 6271, Seq. ID No. 6277, Seq. ID No. 6279, Seq. ID No. 6281, Seq. ID No. 6287, Seq. ID No. 6291, Seq. ID No. 6293, Seq. ID No. 6295, Seq. ID No. 6297, Seq. ID No. 6299, Seq. ID No. 6305, Seq. ID No. 6307, Seq. ID No. 6309, Seq. ID No. 6311, Seq. ID No. 6313, Seq. ID No. 6315, Seq. ID No. 6317, Seq. ID No. 6319, Seq. ID No. 6321, Seq. ID No. 6323, Seq. ID No. 6329, Seq. ID No. 6331, Seq. ID No. 6333, Seq. ID No. 6335, Seq. ID No. 6337, Seq. ID No. 6339, Seq. ID No. 6341, Seq. ID No. 6343, Seq. ID No. 6345, Seq. ID No. 6347, Seq. ID No. 6349, Seq. ID No. 6351, Seq. ID No. 6353, Seq. ID No. 6355, Seq. ID No. 6357, Seq. ID No. 6359, Seq. ID No. 6361, Seq. ID No. 6367, Seq. ID No. 6369, Seq. ID No. 6371, Seq. ID No. 6373, Seq. ID No. 6375, Seq. ID No. 6379, Seq. ID No. 6385, Seq. ID No. 6387, Seq. ID No. 6389, Seq. ID No. 6391, Seq. ID No. 6393, Seq. ID No. 6395, Seq. ID No. 6397, Seq. ID No. 6399, Seq. ID No. 6401, Seq. ID No. 6403, Seq. ID No. 6405, Seq. ID No. 6407, Seq. ID No. 6409, Seq. ID No. 6411, Seq. ID No. 6413, Seq. ID No. 6417, Seq. ID No. 6419, Seq. ID No. 6421, Seq. ID No. 6423, Seq. ID No. 6433, Seq. ID No. 6435, Seq. ID No. 6437, Seq. ID No. 6439, Seq. ID No. 6441, Seq. ID No. 6443, Seq. ID No. 6445, Seq. ID No. 6447, Seq. ID No. 6449, Seq. ID No. 6451, Seq. ID No. 6453, Seq. ID No. 6455, Seq. ID No. 6457, Seq. ID No. 6459, Seq. ID No. 6461, Seq. ID No. 6463, Seq. ID No. 6465, Seq. ID No. 6467, Seq. ID No. 6469, Seq. ID No. 6471, Seq. ID No. 6475, Seq. ID No. 6477, Seq. ID No. 6481, Seq. ID No. 6483, Seq. ID No. 6485, Seq. ID No. 6487, Seq. ID No. 6489, Seq. ID No. 6491, Seq. ID No. 6493, Seq. ID No. 6495, Seq. ID No. 6499, Seq. ID No. 6501, Seq. ID No. 6503, Seq. ID No. 6505, Seq. ID No. 6507, Seq. ID No. 6509, Seq. ID No. 6511, Seq. ID No. 6513, Seq. ID No. 6515, Seq. ID No. 6519, Seq. ID No. 6521, Seq. ID No. 6523, Seq. ID No. 6525, Seq. ID No. 6527, Seq. ID No. 6529, Seq. ID No. 6531, Seq. ID No. 6533, Seq. ID No. 6535, Seq. ID No. 6537, Seq. ID No. 6539, Seq. ID No. 6545, Seq. ID No. 6547, Seq. ID No. 6549, Seq. ID No. 6551, Seq. ID No. 6553, Seq. ID No. 6559, Seq. ID No. 6561, Seq. ID No. 6563, Seq. ID No. 6565, Seq. ID No. 6567, Seq. ID No. 6569, Seq. ID No. 6575, Seq. ID No. 6577, Seq. ID No. 6579, Seq. ID No. 6583, Seq. ID No. 6585, Seq. ID No. 6587, Seq. ID No. 6589, Seq. ID No. 6591, Seq. ID No. 6605, Seq. ID No. 6607, Seq. ID No. 6609, Seq. ID No. 6611, Seq. ID No. 6613, Seq. ID No. 6615, Seq. ID No. 6617, Seq. ID No. 6619, Seq. ID No. 6621, Seq. ID No. 6623, Seq. ID No. 6625, Seq. ID No. 6627, Seq. ID No. 6629, Seq. ID No. 6633, Seq. ID No. 6635, Seq. ID No. 6641, and Seq. ID No. 6647.

In yet another preferred embodiment, the invention comprises an isolated polynucleotide of the Alloiococcus otitidis genomic sequence encoding a polypeptide, which was identified by BlastP analysis, and the polynucleotide is selected from one of Seq. ID No. 9, Seq. ID No. 17, Seq. ID No. 45, Seq. ID No. 49, Seq. ID No. 53, Seq. ID No. 59, Seq. ID No. 89, Seq. ID No. 145, Seq. ID No. 157, Seq. ID No. 167, Seq. ID No. 171, Seq. ID No. 191, Seq. ID No. 215, Seq. ID No. 217, Seq. ID No. 225, Seq. ID No. 239, Seq. ID No. 253, Seq. ID No. 273, Seq. ID No. 277, Seq. ID No. 289, Seq. ID No. 329, Seq. ID No. 343, Seq. ID No. 345, Seq. ID No. 375, Seq. ID No. 501, Seq. ID No. 505, Seq. ID No. 511, Seq. ID No. 515, Seq. ID No. 529, Seq. ID No. 569, Seq. ID No. 575, Seq. ID No. 593, Seq. ID No. 595, Seq. ID No. 637, Seq. ID No. 659, Seq. ID No. 669, Seq. ID No. 675, Seq. ID No. 681, Seq. ID No. 729, Seq. ID No. 779, Seq. ID No. 819, Seq. ID No. 895, Seq. ID No. 921, Seq. ID No. 935, Seq. ID No. 955, Seq. ID No. 967, Seq. ID No. 985, Seq. ID No. 1017, Seq. ID No. 1039, Seq. ID No. 1075, Seq. ID No. 1089, Seq. ID No. 1137, Seq. ID No. 1223, Seq. ID No. 1241, Seq. ID No. 1315, Seq. ID No. 1331, Seq. ID No. 1369, Seq. ID No. 1459, Seq. ID No. 1517, Seq. ID No. 1561, Seq. ID No. 1563, Seq. ID No. 1567, Seq. ID No. 1571, Seq. ID No. 1573, Seq. ID No. 1601, Seq. ID No. 1607, Seq. ID No. 1613, Seq. ID No. 1619, Seq. ID No. 1641, Seq. ID No. 1647, Seq. ID No. 1653, Seq. ID No. 1657, Seq. ID No. 1665, Seq. ID No. 1699, Seq. ID No. 1713, Seq. ID No. 1751, Seq. ID No. 1771, Seq. ID No. 1785, Seq. ID No. 1789, Seq. ID No. 1791, Seq. ID No. 1797, Seq. ID No. 1811, Seq. ID No. 1817, Seq. ID No. 1819, Seq. ID No. 1821, Seq. ID No. 1871, Seq. ID No. 1895, Seq. ID No. 1905, Seq. ID No. 1913, Seq. ID No. 1919, Seq. ID No. 1923, Seq. ID No. 1965, Seq. ID No. 1971, Seq. ID No. 1999, Seq. ID No. 2047, Seq. ID No. 2055, Seq. ID No. 2091, Seq. ID No. 2093, Seq. ID No. 2115, Seq. ID No. 2119, Seq. ID No. 2127, Seq. ID No. 2165, Seq. ID No. 2179, Seq. ID No. 2207, Seq. ID No. 2223, Seq. ID No. 2227, Seq. ID No. 2241, Seq. ID No. 2253, Seq. ID No. 2263, Seq. ID No. 2269, Seq. ID No. 2275, Seq. ID No. 2301, Seq. ID No. 2315, Seq. ID No. 2329, Seq. ID No. 2333, Seq. ID No. 2343, Seq. ID No. 2345, Seq. ID No. 2363, Seq. ID No. 2373, Seq. ID No. 2391, Seq. ID No. 2417, Seq. ID No. 2429, Seq. ID No. 2465, Seq. ID No. 2475, Seq. ID No. 2483, Seq. ID No. 2513, Seq. ID No. 2525, Seq. ID No. 2589, Seq. ID No. 2613, Seq. ID No. 2651, Seq. ID No. 2669, Seq. ID No. 2677, Seq. ID No. 2687, Seq. ID No. 2689, Seq. ID No. 2693, Seq. ID No. 2703, Seq. ID No. 2709, Seq. ID No. 2715, Seq. ID No. 2751, Seq. ID No. 2753, Seq. ID No. 2767, Seq. ID No. 2787, Seq. ID No. 2791, Seq. ID No. 2803, Seq. ID No. 2867, Seq. ID No. 2871, Seq. ID No. 2907, Seq. ID No. 2919, Seq. ID No. 2921, Seq. ID No. 2923, Seq. ID No. 2927, Seq. ID No. 2933, Seq. ID No. 2957, Seq. ID No. 2979, Seq. ID No. 2999, Seq. ID No. 3005, Seq. ID No. 3019, Seq. ID No. 3031, Seq. ID No. 3035, Seq. ID No. 3055, Seq. ID No. 3067, Seq. ID No. 3085, Seq. ID No. 3165, Seq. ID No. 3183, Seq. ID No. 3189, Seq. ID No. 3197, Seq. ID No. 3211, Seq. ID No. 3251, Seq. ID No. 3257, Seq. ID No. 3259, Seq. ID No. 3297, Seq. ID No. 3319, Seq. ID No. 3323, Seq. ID No. 3335, Seq. ID No. 3337, Seq. ID No. 3355, Seq. ID No. 3359, Seq. ID No. 3377, Seq. ID No. 3397, Seq. ID No. 3405, Seq. ID No. 3415, Seq. ID No. 3425, Seq. ID No. 3427, Seq. ID No. 3461, Seq. ID No. 3463, Seq. ID No. 3487, Seq. ID No. 3493, Seq. ID No. 3497, Seq. ID No. 3507, Seq. ID No. 3519, Seq. ID No. 3521, Seq. ID No. 3529, Seq. ID No. 3535, Seq. ID No. 3629, Seq. ID No. 3635, Seq. ID No. 3727, Seq. ID No. 3759, Seq. ID No. 3771, Seq. ID No. 3793, Seq. ID No. 3839, Seq. ID No. 3851, Seq. ID No. 3863, Seq. ID No. 3865, Seq. ID No. 3883, Seq. ID No. 3891, Seq. ID No. 3913, Seq. ID No. 3925, Seq. ID No. 3929, Seq. ID No. 3975, Seq. ID No. 3981, Seq. ID No. 4027, Seq. ID No. 4039, Seq. ID No. 4061, Seq. ID No. 4073, Seq. ID No. 4081, Seq. ID No. 4103, Seq. ID No. 4111, Seq. ID No. 4137, Seq. ID No. 4225, Seq. ID No. 4247, Seq. ID No. 4343, Seq. ID No. 4379, Seq. ID No. 4389, Seq. ID No. 4469, Seq. ID No. 4473, Seq. ID No. 4481, Seq. ID No. 4485, Seq. ID No. 4493, Seq. ID No. 4529, Seq. ID No. 4643, Seq. ID No. 4705, Seq. ID No. 4713, Seq. ID No. 4717, Seq. ID No. 4813, Seq. ID No. 4815, Seq. ID No. 4825, Seq. ID No. 4827, Seq. ID No. 4833, Seq. ID No. 4837, Seq. ID No. 4851, Seq. ID No. 4863, Seq. ID No. 4875, Seq. ID No. 4879, Seq. ID No. 4893, Seq. ID No. 4901, Seq. ID No. 4919, Seq. ID No. 4931, Seq. ID No. 4959, Seq. ID No. 4963, Seq. ID No. 4985, Seq. ID No. 5113, Seq. ID No. 5115, Seq. ID No. 5117, Seq. ID No. 5125, Seq. ID No. 5141, Seq. ID No. 5183, Seq. ID No. 5201, Seq. ID No. 5205, Seq. ID No. 5213, Seq. ID No. 5219, Seq. ID No. 5279, Seq. ID No. 5285, Seq. ID No. 5331, Seq. ID No. 5381, Seq. ID No. 5389, Seq. ID No. 5405, Seq. ID No. 5411, Seq. ID No. 5419, Seq. ID No. 5433, Seq. ID No. 5463, Seq. ID No. 5477, Seq. ID No. 5491, Seq. ID No. 5509, Seq. ID No. 5513, Seq. ID No. 5525, Seq. ID No. 5529, Seq. ID No. 5537, Seq. ID No. 5567, Seq. ID No. 5599, Seq. ID No. 5621, Seq. ID No. 5637, Seq. ID No. 5699, Seq. ID No. 5723, Seq. ID No. 5733, Seq. ID No. 5761, Seq. ID No. 5791, Seq. ID No. 5895, Seq. ID No. 5901, Seq. ID No. 5903, Seq. ID No. 5909, Seq. ID No. 5913, Seq. ID No. 5919, Seq. ID No. 5929, Seq. ID No. 5939, Seq. ID No. 6001, Seq. ID No. 6043, Seq. ID No. 6053, Seq. ID No. 6057, Seq. ID No. 6089, Seq. ID No. 6095, Seq. ID No. 6103, Seq. ID No. 6111, Seq. ID No. 6117, Seq. ID No. 6165, Seq. ID No. 6169, Seq. ID No. 6185, Seq. ID No. 6191, Seq. ID No. 6209, Seq. ID No. 6229, Seq. ID No. 6257, Seq. ID No. 6263, Seq. ID No. 6271, Seq. ID No. 6275, Seq. ID No. 6289, Seq. ID No. 6303, Seq. ID No. 6365, Seq. ID No. 6369, Seq. ID No. 6383, and Seq. ID No. 6415.

In still yet another preferred embodiment, the invention comprises an isolated polynucleotide of the Alloiococcus otitidis genomic sequence encoding a polypeptide identified by Pfam analysis, and the polynucleotide is selected from one of Seq. ID No. 89, Seq. ID No. 145, Seq. ID No. 155, Seq. ID No. 167, Seq. ID No. 173, Seq. ID No. 191, Seq. ID No. 215, Seq. ID No. 219, Seq. ID No. 221, Seq. ID No. 223, Seq. ID No. 225, Seq. ID No. 233, Seq. ID No. 239, Seq. ID No. 251, Seq. ID No. 253, Seq. ID No. 271, Seq. ID No. 291, Seq. ID No. 331, Seq. ID No. 341, Seq. ID No. 345, Seq. ID No. 375, Seq. ID No. 501, Seq. ID No. 505, Seq. ID No. 511, Seq. ID No. 529, Seq. ID No. 567, Seq. ID No. 569, Seq. ID No. 575, Seq. ID No. 593, Seq. ID No. 595, Seq. ID No. 601, Seq. ID No. 637, Seq. ID No. 649, Seq. ID No. 661, Seq. ID No. 669, Seq. ID No. 675, Seq. ID No. 681, Seq. ID No. 683, Seq. ID No. 729, Seq. ID No. 753, Seq. ID No. 779, Seq. ID No. 827, Seq. ID No. 839, Seq. ID No. 895, Seq. ID No. 939, Seq. ID No. 969, Seq. ID No. 985, Seq. ID No. 1015, Seq. ID No. 1039, Seq. ID No. 1075, Seq. ID No. 1137, Seq. ID No. 1155, Seq. ID No. 1163, Seq. ID No. 1175, Seq. ID No. 1223, Seq. ID No. 1231, Seq. ID No. 1237, Seq. ID No. 1241, Seq. ID No. 1261, Seq. ID No. 1331, Seq. ID No. 1339, Seq. ID No. 1369, Seq. ID No. 1477, Seq. ID No. 1517, Seq. ID No. 1531, Seq. ID No. 1539, Seq. ID No. 1549, Seq. ID No. 1563, Seq. ID No. 1567, Seq. ID No. 1571, Seq. ID No. 1573, Seq. ID No. 1589, Seq. ID No. 1595, Seq. ID No. 1647, Seq. ID No. 1657, Seq. ID No. 1771, Seq. ID No. 1785, Seq. ID No. 1789, Seq. ID No. 1819, Seq. ID No. 1821, Seq. ID No. 1969, Seq. ID No. 2015, Seq. ID No. 2047, Seq. ID No. 2087, Seq. ID No. 2089, Seq. ID No. 2093, Seq. ID No. 211.5, Seq. ID No. 2117, Seq. ID No. 2127, Seq. ID No. 2141, Seq. ID No. 2179, Seq. ID No. 2207, Seq. ID No. 2223, Seq. ID No. 2253, Seq. ID No. 2275, Seq. ID No. 2301, Seq. ID No. 2315, Seq. ID No. 2329, Seq. ID No. 2343, Seq. ID No. 2349, Seq. ID No. 2363, Seq. ID No. 2381, Seq. ID No. 2391, Seq. ID No. 2417, Seq. ID No. 2421, Seq. ID No. 2423, Seq. ID No. 2429, Seq. ID No. 2481, Seq. ID No. 2487, Seq. ID No. 2489, Seq. ID No. 2491, Seq. ID No. 2509, Seq. ID No. 2519, Seq. ID No. 2525, Seq. ID No. 2547, Seq. ID No. 2553, Seq. ID No. 2563, Seq. ID No. 2587, Seq. ID No. 2599, Seq. ID No. 2607, Seq. ID No. 2613, Seq. ID No. 2651, Seq. ID No. 2675, Seq. ID No. 2681, Seq. ID No. 2687, Seq. ID No. 2693, Seq. ID No. 2699, Seq. ID No. 2703, Seq. ID No. 2709, Seq. ID No. 2711, Seq. ID No. 2727, Seq. ID No. 2753, Seq. ID No. 2819, Seq. ID No. 2821, Seq. ID No. 2823, Seq. ID No. 2867, Seq. ID No. 2871, Seq. ID No. 2907, Seq. ID No. 2919, Seq. ID No. 2957, Seq. ID No. 2971, Seq. ID No. 2979, Seq. ID No. 2999, Seq. ID No. 3005, Seq. ID No. 3019, Seq. ID No. 3035, Seq. ID No. 3065; Seq. ID No. 3085, Seq. ID No. 3149, Seq. ID No. 3165, Seq. ID No. 3183, Seq. ID No. 3203, Seq. ID No. 3211, Seq. ID No. 3251, Seq. ID No. 3259, Seq. ID No. 3319, Seq. ID No. 3323, Seq. ID No. 3335, Seq. ID No. 3337, Seq. ID No. 3347, Seq. ID No. 3359, Seq. ID No. 3377, Seq. ID No. 3379, Seq. ID No. 3399, Seq. ID No. 3425, Seq. ID No. 3483, Seq. ID No. 3495, Seq. ID No. 3497, Seq. ID No. 3519, Seq. ID No. 3529, Seq. ID No. 3599, Seq. ID No. 3611, Seq. ID No. 3615, Seq. ID No. 3619, Seq. ID No. 3663, Seq. ID No. 3739, Seq. ID No. 3759, Seq. ID No. 3771, Seq. ID No. 3797, Seq. ID No. 3825, Seq. ID No. 3841, Seq. ID No. 3855, Seq. ID No. 3863, Seq. ID No. 3901, Seq. ID No. 3929, Seq. ID No. 4015, Seq. ID No. 4017, Seq. ID No. 4055, Seq. ID No. 4075, Seq. ID No. 4085, Seq. ID No. 4089, Seq. ID No. 4099, Seq. ID No. 4105, Seq. ID No. 4129, Seq. ID No. 4203, Seq. ID No. 4243, Seq. ID No. 4251, Seq. ID No. 4343, Seq. ID No. 4383, Seq. ID No. 4475, Seq. ID No. 4481, Seq. ID No. 4487, Seq. ID No. 4491, Seq. ID No. 4533, Seq. ID No. 4627, Seq. ID No. 4705, Seq. ID No. 4713, Seq. ID No. 4717, Seq. ID No. 4719, Seq. ID No. 4729, Seq. ID No. 4789, Seq. ID No. 4791, Seq. ID No. 4793, Seq. ID No. 4803, Seq. ID No. 4813, Seq. ID No. 4815, Seq. ID No. 4825, Seq. ID No. 4833, Seq. ID No. 4837, Seq. ID No. 4875, Seq. ID No. 4879, Seq. ID No. 4919, Seq. ID No. 4931, Seq. ID No. 4947, Seq. ID No. 4985, Seq. ID No. 5077, Seq. ID No. 5115, Seq. ID No. 5117, Seq. ID No. 5127, Seq. ID No. 5141, Seq. ID No. 5193, Seq. ID No. 5215, Seq. ID No. 5219, Seq. ID No. 5275, Seq. ID No. 5381, Seq. ID No. 5389, Seq. ID No. 5409, Seq. ID No. 5411, Seq. ID No. 5475, Seq. ID No. 5491, Seq. ID No. 5513, Seq. ID No. 5519, Seq. ID No. 5525, Seq. ID No. 5529, Seq. ID No. 5537, Seq. ID No. 5557, Seq. ID No. 5581, Seq. ID No. 5599, Seq. ID No. 5637, Seq. ID No. 5685, Seq. ID No. 5723, Seq. ID No. 5755, Seq. ID No. 5761, Seq. ID No. 5895, Seq. ID No. 5901, Seq. ID No. 5903, Seq. ID No. 5911, Seq. ID No. 5913, Seq. ID No. 5919, Seq. ID No. 5923, Seq. ID No. 5929, Seq. ID No. 6001, Seq. ID No. 6043, Seq. ID No. 6053, Seq. ID No. 6055, Seq. ID No. 6089, Seq. ID No. 6095, Seq. ID No. 6111, Seq. ID No. 6117, Seq. ID No. 6165, Seq. ID No. 6169, Seq. ID No. 6187, Seq. ID No. 6195, Seq. ID No. 6219, Seq. ID No. 6233, Seq. ID No. 6269, Seq. ID No. 6275, Seq. ID No. 6279, Seq. ID No. 6287, Seq. ID No. 6297, Seq. ID No. 6299, Seq. ID No. 6305, Seq. ID No. 6311, Seq. ID No. 6369, Seq. ID No. 6379, Seq. ID No. 6587, and Seq. ID No. 6591.

In another preferred embodiment, the invention comprises an isolated polynucleotide of the Alloiococcus otitidis genomic sequence encoding a polypeptide, and which has been identified using the proteomic methods used for studying surface proteins of Streptococcus pneumoniae, and the polynucleotide is selected from one of Seq. ID No. 1, Seq. ID No. 5, Seq. ID No. 93, Seq. ID No. 95, Seq. ID No. 103, Seq. ID No. 107, Seq. ID No. 157, Seq. ID No. 223, Seq. ID No. 225, Seq. ID No. 233, Seq. ID No. 239, Seq. ID No. 253, Seq. ID No. 317, Seq. ID No. 321, Seq. ID No. 341, Seq. ID No. 345, Seq. ID No. 353, Seq. ID No. 361, Seq. ID No. 363, Seq. ID No. 365, Seq. ID No. 375, Seq. ID No. 379, Seq. ID No. 383, Seq. ID No. 389, Seq. ID No. 391, Seq. ID No. 397, Seq. ID No. 405, Seq. ID No. 409, Seq. ID No. 415, Seq. ID No. 423, Seq. ID No. 425, Seq. ID No. 427, Seq. ID No. 429, Seq. ID No. 435, Seq. ID No. 437, Seq. ID No. 439, Seq. ID No. 441, Seq. ID No. 443, Seq. ID No. 445, Seq. ID No. 447, Seq. ID No. 449, Seq. ID No. 461, Seq. ID No. 463, Seq. ID No. 465, Seq. ID No. 513, Seq. ID No. 543, Seq. ID No. 551, Seq. ID No. 553, Seq. ID No. 559, Seq. ID No. 595, Seq. ID No. 601, Seq. ID No. 637, Seq. ID No. 649, Seq. ID No. 657, Seq. ID No. 669, Seq. ID No. 675, Seq. ID No. 683, Seq. ID No. 753, Seq. ID No. 929, Seq. ID No. 969, Seq. ID No. 1041, Seq. ID No. 1043, Seq. ID No. 1085, Seq. ID No. 1089, Seq. ID No. 1093, Seq. ID No. 1097, Seq. ID No. 1099, Seq. ID No. 1101, Seq. ID No. 1175, Seq. ID No. 1205, Seq. ID No. 1237, Seq. ID No. 1459, Seq. ID No. 1495, Seq. ID No. 1497, Seq. ID No. 1503, Seq. ID No. 1517, Seq. ID No. 1527, Seq. ID No. 1531, Seq. ID No. 1539, Seq. ID No. 1543, Seq. ID No. 1549, Seq. ID No. 1573, Seq. ID No. 1657, Seq. ID No. 1709, Seq. ID No. 1767, Seq. ID No. 1785, Seq. ID No. 1821, Seq. ID No. 1889, Seq. ID No. 1925, Seq. ID No. 1969, Seq. ID No. 2003, Seq. ID No. 2047, Seq. ID No. 2075, Seq. ID No. 2089, Seq. ID No. 2093, Seq. ID No. 2117, Seq. ID No. 2137, Seq. ID No. 2143, Seq. ID No. 2205, Seq. ID No. 2235, Seq. ID No. 2257, Seq. ID No. 2301, Seq. ID No. 2363, Seq. ID No. 2423, Seq. ID No. 2445, Seq. ID No. 2451, Seq. ID No. 2481, Seq. ID No. 2495, Seq. ID No. 2505, Seq. ID No. 2577, Seq. ID No. 2587, Seq. ID No. 2613, Seq. ID No. 2633, Seq. ID No. 2651, Seq. ID No. 2665, Seq. ID No. 2669, Seq. ID No. 2677, Seq. ID No. 2687, Seq. ID No. 2693, Seq. ID No. 2699, Seq. ID No. 2711, Seq. ID No. 2715, Seq. ID No. 2757, Seq. ID No. 2791, Seq. ID No. 2793, Seq. ID No. 2803, Seq. ID No. 2867, Seq. ID No. 2919, Seq. ID No. 2929, Seq. ID No. 3005, Seq. ID No. 3019, Seq. ID No. 3125, Seq. ID No. 3137, Seq. ID No. 3161, Seq. ID No. 3179, Seq. ID No. 3251, Seq. ID No. 3259, Seq. ID No. 3285, Seq. ID No. 3309, Seq. ID No. 3317, Seq. ID No. 3337, Seq. ID No. 3345, Seq. ID No. 3359, Seq. ID No. 3475, Seq. ID No. 3515, Seq. ID No. 3521, Seq. ID No. 3641, Seq. ID No. 3695, Seq. ID No. 3793, Seq. ID No. 3901, Seq. ID No. 3927, Seq. ID No. 3987, Seq. ID No. 4001, Seq. ID No. 4057, Seq. ID No. 4081, Seq. ID No. 4111, Seq. ID No. 4125, Seq. ID No. 4193, Seq. ID No. 4195, Seq. ID No. 4237, Seq. ID No. 4247, Seq. ID No. 4383, Seq. ID No. 4393, Seq. ID No. 4397, Seq. ID No. 4401, Seq. ID No. 4435, Seq. ID No. 4449, Seq. ID No. 4471, Seq. ID No. 4491, Seq. ID No. 4511, Seq. ID No. 4519, Seq. ID No. 4531, Seq. ID No. 4549, Seq. ID No. 4577, Seq. ID No. 4581, Seq. ID No. 4585, Seq. ID No. 4609, Seq. ID No. 4611, Seq. ID No. 4613, Seq. ID No. 4675, Seq. ID No. 4685, Seq. ID No. 4719, Seq. ID No. 4729, Seq. ID No. 4743, Seq. ID No. 4755, Seq. ID No. 4781, Seq. ID No. 4787, Seq. ID No. 4837, Seq. ID No. 4841, Seq. ID No. 4849, Seq. ID No. 4855, Seq. ID No. 4889, Seq. ID No. 4901, Seq. ID No. 4915, Seq. ID No. 4917, Seq. ID No. 4931, Seq. ID No. 4935, Seq. ID No. 4977, Seq. ID No. 5025, Seq. ID No. 5029, Seq. ID No. 5109, Seq. ID No. 5115, Seq. ID No. 5117, Seq. ID No. 5155, Seq. ID No. 5185, Seq. ID No. 5201, Seq. ID No. 5203, Seq. ID No. 5219, Seq. ID No. 5261, Seq. ID No. 5289, Seq. ID No. 5295, Seq. ID No. 5377, Seq. ID No. 5389, Seq. ID No. 5399, Seq. ID No. 5411, Seq. ID No. 5537, Seq. ID No. 5621, Seq. ID No. 5627, Seq. ID No. 5657, Seq. ID No. 5675, Seq. ID No. 5717, Seq. ID No. 5777, Seq. ID No. 5859, Seq. ID No. 5901, Seq. ID No. 5913, Seq. ID No. 5919, Seq. ID No. 5925, Seq. ID No. 5933, Seq. ID No. 6055, Seq. ID No. 6129, Seq. ID No. 6133, Seq. ID No. 6187, Seq. ID No. 6193, Seq. ID No. 6219, Seq. ID No. 6271, Seq. ID No. 6277, Seq. ID No. 6369, Seq. ID No. 6375, Seq. ID No. 6379, Seq. ID No. 6389, Seq. ID No. 6591, and Seq. ID No. 6605.

In another preferred embodiment, the invention comprises an isolated polynucleotide of the Alloiococcus otitidis genomic sequence encoding a polypeptide, wherein the polypeptide encoded by the polynucleotide was identified using HMM LPXTG motif finder as having a LPXTG motif, and wherein the polypeptide is covalently attached to the peptidoglycan layer, and the polynucleotide is selected from one Seq. ID No. 503, Seq. ID No. 505, Seq. ID No. 1315, Seq. ID No. 1317, Seq. ID No. 1363, Seq. ID No. 1365, Seq. ID No. 1367, Seq. ID No. 1369, Seq. ID No. 1559, Seq. ID No. 1561, Seq. ID No. 1581, Seq. ID No. 1739, Seq. ID No. 1741, Seq. ID No. 2811, Seq. ID No. 2813, Seq. ID No. 3619, Seq. ID No. 5587, Seq. ID No. 5589, Seq. ID No. 5591, Seq. ID No. 5781, Seq. ID No. 5783, and Seq. ID No. 6103.

In another preferred embodiment, the invention comprises an isolated polynucleotide of the Alloiococcus otitidis genomic sequence encoding a lipoprotein as predicted by HMM Lipo software program, and the polynucleotide is selected from Seq. ID No. 45, Seq. ID No. 47, Seq. ID No. 171, Seq. ID No. 225, Seq. ID No. 419, Seq. ID No. 513, Seq. ID No. 575, Seq. ID No. 591, Seq. ID No. 593, Seq. ID No. 657, Seq. ID No. 659, Seq. ID No. 669, Seq. ID No. 675, Seq. ID No. 745, Seq. ID No. 747, Seq. ID No. 935, Seq. ID No. 1045, Seq. ID No. 1075, Seq. ID No. 1137, Seq. ID No. 1173, Seq. ID No. 1191, Seq. ID No. 1193, Seq. ID No. 1195, Seq. ID No. 1563, Seq. ID No. 1661, Seq. ID No. 1663, Seq. ID No. 1665, Seq. ID No. 1791, Seq. ID No. 1797, Seq. ID No. 1917, Seq. ID No. 1969, Seq. ID No. 2115, Seq. ID No. 2159, Seq. ID No. 2429, Seq. ID No. 2527, Seq. ID No. 2699, Seq. ID No. 2815, Seq. ID No. 2875, Seq. ID No. 2975, Seq. ID No. 2977, Seq. ID No. 2991, Seq. ID No. 3043, Seq. ID No. 3323, Seq. ID No. 3337, Seq. ID No. 4133, Seq. ID No. 4137, Seq. ID No. 4481, Seq. ID No. 4705, Seq. ID No. 4827, Seq. ID No. 4869, Seq. ID No. 5115, Seq. ID No. 5377, Seq. ID No. 5379, Seq. ID No. 5491, Seq. ID No. 5509, Seq. ID No. 5513, Seq. ID No. 5525, Seq. ID No. 5529, Seq. ID No. 5531, Seq. ID No. 5889, Seq. ID No. 5909, Seq. ID No. 6025, Seq. ID No. 6027, Seq. ID No. 6087, Seq. ID No. 6089, Seq. ID No. 6325, Seq. ID No. 6327, Seq. ID No. 6415, Seq. ID No. 6637, Seq. ID No. 6639, and Seq. ID No. 6645.

In another preferred embodiment, the invention comprises an isolated polynucleotide of the Alloiococcus otitidis genomic sequence encoding a polypeptide, wherein the polypeptide encoded by the polynucleotide is predicted by HMM program to be non-covalently bound to the peptidoglycan layer, and the polynucleotide is selected from Seq. ID No. 3589, Seq. ID No. 3987, Seq. ID No. 5219, and Seq. ID No. 5337.

In another preferred embodiment, the invention comprises an isolated polynucleotide of the Alloiococcus otitidis genomic sequence encoding a polypeptide, wherein the polypeptide encodes a polypeptide having a RGDX motif wherein X is not a proline, and the polynucleotide is selected from Seq. ID No. 37, Seq. ID No. 99, Seq. ID No. 107, Seq. ID No. 109, Seq. ID No. 111, Seq. ID No. 351, Seq. ID No. 353, Seq. ID No. 465, Seq. ID No. 467, Seq. ID No. 469, Seq. ID No. 471, Seq. ID No. 683, Seq. ID No. 821, Seq. ID No. 823, Seq. ID No. 1021, Seq. ID No. 1023, Seq. ID No. 1157, Seq. ID No. 1159, Seq. ID No. 1233, Seq. ID No. 1333, Seq. ID No. 1335, Seq. ID No. 1449, Seq. ID No. 1559, Seq. ID No. 1561, Seq. ID No. 1563, Seq. ID No. 1625, Seq. ID No. 1777, Seq. ID No. 1827, Seq. ID No. 1829, Seq. ID No. 1871, Seq. ID No. 1925, Seq. ID No. 2135, Seq. ID No. 2145, Seq. ID No. 2173, Seq. ID No. 2175, Seq. ID No. 2177, Seq. ID No. 2179, Seq. ID No. 2259, Seq. ID No. 2317, Seq. ID No. 2319, Seq. ID No. 2321, Seq. ID No. 2323, Seq. ID No. 2325, Seq. ID No. 2327, Seq. ID No. 2329, Seq. ID No. 2401, Seq. ID No. 2671, Seq. ID No. 2863, Seq. ID No. 2869, Seq. ID No. 2919, Seq. ID No. 2921, Seq. ID No. 2993, Seq. ID No. 3087, Seq. ID No. 3137, Seq. ID No. 3279, Seq. ID No. 3281, Seq. ID No. 3283, Seq. ID No. 3333, Seq. ID No. 3335, Seq. ID No. 3497, Seq. ID No. 3499, Seq. ID No. 3501, Seq. ID No. 3533, Seq. ID No. 3671, Seq. ID No. 3673, Seq. ID No. 3675, Seq. ID No. 3837, Seq. ID No. 3839, Seq. ID No. 3841, Seq. ID No. 3843, Seq. ID No. 3929, Seq. ID No. 3961, Seq. ID No. 4037, Seq. ID No. 4193, Seq. ID No. 4239, Seq. ID No. 4327, Seq. ID No. 4329, Seq. ID No. 4333, Seq. ID No. 4393, Seq. ID No. 4463, Seq. ID No. 4465, Seq. ID No. 4467, Seq. ID No. 4597, Seq. ID No. 4629, Seq. ID No. 4631, Seq. ID No. 4675, Seq. ID No. 4677, Seq. ID No. 4679, Seq. ID No. 4689, Seq. ID No. 4691, Seq. ID No. 4693, Seq. ID No. 4781, Seq. ID No. 4799, Seq. ID No. 4801, Seq. ID No. 4803, Seq. ID No. 4861, Seq. ID No. 5009, Seq. ID No. 5143, Seq. ID No. 5145, Seq. ID No. 5179, Seq. ID No. 5181, Seq. ID No. 5183, Seq. ID No. 5249, Seq. ID No. 5251, Seq. ID No. 5253, Seq. ID No. 5259, Seq. ID No. 5261, Seq. ID No. 5293, Seq. ID No. 5295, Seq. ID No. 5297, Seq. ID No. 5299, Seq. ID No. 5307, Seq. ID No. 5309, Seq. ID No. 5411, Seq. ID No. 5535, Seq. ID No. 5537, Seq. ID No. 5745, Seq. ID No. 5821, Seq. ID No. 5823, Seq. ID No. 5825, Seq. ID No. 6029, Seq. ID No. 6171, Seq. ID No. 6307, Seq. ID No. 6309, and Seq. ID No. 6311.

In another preferred embodiment, the polynucleotide is selected from the Alloiococcus otitidis polynucleotides that are predicted by BlastP analysis as encoding polypeptides involved in capsule biosynthesis and transport, and the polynucleotide is selected from Seq. ID No. 49, Seq. ID No. 51, Seq. ID No. 53, Seq. ID No. 59, Seq. ID No. 195, Seq. ID No. 689, Seq. ID No. 703, Seq. ID No. 925, Seq. ID No. 1597, Seq. ID No. 1601, Seq. ID No. 1607, Seq. ID No. 1611, Seq. ID No. 1613, Seq. ID No. 1713, Seq. ID No. 2029, Seq. ID No. 2263, Seq. ID No. 2269, Seq. ID No. 2373, Seq. ID No. 2437, Seq. ID No. 2651, Seq. ID No. 2767, Seq. ID No. 2907, Seq. ID No. 3397, Seq. ID No. 3399, Seq. ID No. 3479, Seq. ID No. 3487, Seq. ID No. 3491, Seq. ID No. 3493, Seq. ID No. 3495, Seq. ID No. 3497, Seq. ID No. 3519, Seq. ID No. 3521, Seq. ID No. 3529, Seq. ID No. 3535, Seq. ID No. 3561, Seq. ID No. 3883, Seq. ID No. 3891, Seq. ID No. 3981, and Seq. ID No. 4087.

In yet another preferred embodiment, the polynucleotide is selected from the Alloiococcus otitidis polynucleotides that encodes a polypeptide identified by BlastP analysis as being localized within the capsular loci of the Alloiococcus otitidis genome, and the polynucleotide is selected from one of Seq. ID No. 45, Seq. ID No. 47, Seq. ID No. 51, Seq. ID No. 53, Seq. ID No. 55, Seq. ID No. 57, Seq. ID No. 59, Seq. ID No. 683, Seq. ID No. 685, Seq. ID No. 687, Seq. ID No. 689, Seq. ID No. 691, Seq. ID No. 693, Seq. ID No. 695, Seq. ID No. 697, Seq. ID No. 699, Seq. ID No. 701, Seq. ID No. 703, Seq. ID No. 705, Seq. ID No. 707, Seq. ID No. 709, Seq. ID No. 711, Seq. ID No. 713, Seq. ID No. 715, Seq. ID No. 717, Seq. ID No. 719, Seq. ID No. 721, Seq. ID No. 723, Seq. ID No. 725, Seq. ID No. 727, Seq. ID No. 729, Seq. ID No. 731, Seq. ID No. 733, Seq. ID No. 735, Seq. ID No. 737, Seq. ID No. 739, Seq. ID No. 741, Seq. ID No. 743, Seq. ID No. 745, Seq. ID No. 747, Seq. ID No. 749, Seq. ID No. 751, Seq. ID No. 753, Seq. ID No. 3389, Seq. ID No. 3391, Seq. ID No. 3393, Seq. ID No. 3395, Seq. ID No. 3397, Seq. ID No. 3399, Seq. ID No. 3401, Seq. ID No. 3403, Seq. ID No. 3405, Seq. ID No. 3407, Seq. ID No. 3409, Seq. ID No. 3411, Seq. ID No. 3413, Seq. ID No. 3415, Seq. ID No. 3417, Seq. ID No. 3419, Seq. ID No. 3421, Seq. ID No. 3423, Seq. ID No. 3425, Seq. ID No. 3427, Seq. ID No. 3429, Seq. ID No. 3431, Seq. ID No. 3433, Seq. ID No. 3435, Seq. ID No. 3437, Seq. ID No. 3439, Seq. ID No. 3441, Seq. ID No. 3443, Seq. ID No. 3445, Seq. ID No. 3447, Seq. ID No. 3449, Seq. ID No. 3451, Seq. ID No. 3453, Seq. ID No. 3455, Seq. ID No. 3457, Seq. ID No. 3459, Seq. ID No. 3461, Seq. ID No. 3463, Seq. ID No. 3465, Seq. ID No. 3467, Seq. ID No. 3469, Seq. ID No. 3471, Seq. ID No. 3473, Seq. ID No. 3475, Seq. ID No. 3477, Seq. ID No. 3479, Seq. ID No. 3481, Seq. ID No. 3483, Seq. ID No. 3485, Seq. ID No. 3487, Seq. ID No. 3489, Seq. ID No. 3491, Seq. ID No. 3493, Seq. ID No. 3495, Seq. ID No. 3497, Seq. ID No. 3499, Seq. ID No. 3501, Seq. ID No. 3503, Seq. ID No. 3505, Seq. ID No. 3507, Seq. ID No. 3509, Seq. ID No. 3511, Seq. ID No. 3513, Seq. ID No. 3515, Seq. ID No. 3517, Seq. ID No. 3519, Seq. ID No. 3521, Seq. ID No. 3523, Seq. ID No. 3525, Seq. ID No. 3527, Seq. ID No. 3529, Seq. ID No. 3531, Seq. ID No. 3533, Seq. ID No. 3535, Seq. ID No. 3537, Seq. ID No. 3539, Seq. ID No. 3541, Seq. ID No. 3543, Seq. ID No. 3545, Seq. ID No. 3547, Seq. ID No. 3549, Seq. ID No. 3551, Seq. ID No. 3553, Seq. ID No. 3555, Seq. ID No. 3557, Seq. ID No. 3559, and Seq. ID No. 3561.

In another preferred embodiment, the polynucleotide is selected from the Alloiococcus otitidis polynucleotides identified by BlastP analysis as encoding polypeptides, which are associated with sporulation, and the polynucleotide is selected from one of Seq. ID No. 1137, Seq. ID No. 1455, Seq. ID No. 2137, Seq. ID No. 2141, Seq. ID No. 2165, Seq. ID No. 2179, Seq. ID No. 2301, Seq. ID No. 2753, Seq. ID No. 4211, Seq. ID No. 4251, Seq. ID No. 4327, Seq. ID No. 4569, Seq. ID No. 4719, and Seq. ID No. 4729.

In another preferred embodiment, the polynucleotide is selected from the Alloiococcus otitidis polynucleotides identified by BlastP analysis as being unique and having a BlastP ‘E’ value of >e⁻¹⁰, and the polynucleotide is selected from one of Seq. ID No. 7, Seq. ID No. 19, Seq. ID No. 21, Seq. ID No. 23, Seq. ID No. 31, Seq. ID No. 39, Seq. ID No. 65, Seq. ID No. 67, Seq. ID No. 69, Seq. ID No. 91, Seq. ID No. 99, Seq. ID No. 105, Seq. ID No. 113, Seq. ID No. 115, Seq. ID No. 123, Seq. ID No. 125, Seq. ID No. 127, Seq. ID No. 131, Seq. ID No. 133, Seq. ID No. 139, Seq. ID No. 151, Seq. ID No. 159, Seq. ID No. 161, Seq. ID No. 163, Seq. ID No. 165, Seq. ID No. 169, Seq. ID No. 185, Seq. ID No. 189, Seq. ID No. 193, Seq. ID No. 229, Seq. ID No. 231, Seq. ID No. 255, Seq. ID No. 257, Seq. ID No. 259, Seq. ID No. 275, Seq. ID No. 337, Seq. ID No. 339, Seq. ID No. 347, Seq. ID No. 349, Seq. ID No. 357, Seq. ID No. 359, Seq. ID No. 369, Seq. ID No. 371, Seq. ID No. 377, Seq. ID No. 385, Seq. ID No. 387, Seq. ID No. 393, Seq. ID No. 399, Seq. ID No. 401, Seq. ID No. 403, Seq. ID No. 407, Seq. ID No. 419, Seq. ID No. 431, Seq. ID No. 477, Seq. ID No. 495, Seq. ID No. 507, Seq. ID No. 519, Seq. ID No. 541, Seq. ID No. 545, Seq. ID No. 547, Seq. ID No. 549, Seq. ID No. 557, Seq. ID No. 589, Seq. ID No. 617, Seq. ID No. 619, Seq. ID No. 633, Seq. ID No. 639, Seq. ID No. 641, Seq. ID No. 643, Seq. ID No. 645, Seq. ID No. 647, Seq. ID No. 651, Seq. ID No. 653, Seq. ID No. 655, Seq. ID No. 667, Seq. ID No. 691, Seq. ID No. 693, Seq. ID No. 695, Seq. ID No. 697, Seq. ID No. 715, Seq. ID No. 717, Seq. ID No. 719, Seq. ID No. 731, Seq. ID No. 733, Seq. ID No. 741, Seq. ID No. 743, Seq. ID No. 745, Seq. ID No. 747, Seq. ID No. 749, Seq. ID No. 751, Seq. ID No. 755, Seq. ID No. 757, Seq. ID No. 759, Seq. ID No. 761, Seq. ID No. 763, Seq. ID No. 765, Seq. ID No. 767, Seq. ID No. 801, Seq. ID No. 803, Seq. ID No. 805, Seq. ID No. 807, Seq. ID No. 809, Seq. ID No. 811, Seq. ID No. 813, Seq. ID No. 833, Seq. ID No. 841, Seq. ID No. 849, Seq. ID No. 851, Seq. ID No. 853, Seq. ID No. 855, Seq. ID No. 887, Seq. ID No. 889, Seq. ID No. 891, Seq. ID No. 903, Seq. ID No. 905, Seq. ID No. 909, Seq. ID No. 937, Seq. ID No. 941, Seq. ID No. 943, Seq. ID No. 951, Seq. ID No. 957, Seq. ID No. 973, Seq. ID No. 975, Seq. ID No. 987, Seq. ID No. 1005, Seq. ID No. 1007, Seq. ID No. 1009, Seq. ID No. 1011, Seq. ID No. 1019, Seq. ID No. 1025, Seq. ID No. 1027, Seq. ID No. 1045, Seq. ID No. 1047, Seq. ID No. 1071, Seq. ID No. 1073, Seq. ID No. 1077, Seq. ID No. 1105, Seq. ID No. 1107, Seq. ID No. 1111, Seq. ID No. 1131, Seq. ID No. 1135, Seq. ID No. 1139, Seq. ID No. 1151, Seq. ID No. 1153, Seq. ID No. 1165, Seq. ID No. 1167, Seq. ID No. 1169, Seq. ID No. 1171, Seq. ID No. 1173, Seq. ID No. 1181, Seq. ID No. 1183, Seq. ID No. 1185, Seq. ID No. 1187, Seq. ID No. 1189, Seq. ID No. 1191, Seq. ID No. 1193, Seq. ID No. 1195, Seq. ID No. 1197, Seq. ID No. 1199, Seq. ID No. 1201, Seq. ID No. 1203, Seq. ID No. 1207, Seq. ID No. 1213, Seq. ID No. 1227, Seq. ID No. 1229, Seq. ID No. 1239, Seq. ID No. 1243, Seq. ID No. 1245, Seq. ID No. 1247, Seq. ID No. 1249, Seq. ID No. 1259, Seq. ID No. 1265, Seq. ID No. 1267, Seq. ID No. 1269, Seq. ID No. 1271, Seq. ID No. 1273, Seq. ID No. 1275, Seq. ID No. 1277, Seq. ID No. 1279, Seq. ID No. 1281, Seq. ID No. 1283, Seq. ID No. 1285, Seq. ID No. 1287, Seq. ID No. 1289, Seq. ID No. 1291, Seq. ID No. 1293, Seq. ID No. 1295, Seq. ID No. 1297, Seq. ID No. 1299, Seq. ID No. 1301, Seq. ID No. 1303, Seq. ID No. 1305, Seq. ID No. 1307, Seq. ID No. 1309, Seq. ID No. 1311, Seq. ID No. 1313, Seq. ID No. 1319, Seq. ID No. 1321, Seq. ID No. 1323, Seq. ID No. 1325, Seq. ID No. 1327, Seq. ID No. 1337, Seq. ID No. 1341, Seq. ID No. 1343, Seq. ID No. 1347, Seq. ID No. 1349, Seq. ID No. 1351, Seq. ID No. 1353, Seq. ID No. 1355, Seq. ID No. 1371, Seq. ID No. 1379, Seq. ID No. 1381, Seq. ID No. 1389, Seq. ID No. 1397, Seq. ID No. 1399, Seq. ID No. 1401, Seq. ID No. 1417, Seq. ID No. 1419, Seq. ID No. 1421, Seq. ID No. 1423, Seq. ID No. 1439, Seq. ID No. 1445, Seq. ID No. 1447, Seq. ID No. 1457, Seq. ID No. 1465, Seq. ID No. 1479, Seq. ID No. 1511, Seq. ID No. 1551, Seq. ID No. 1553, Seq. ID No. 1555, Seq. ID No. 1557, Seq. ID No. 1579, Seq. ID No. 1609, Seq. ID No. 1621, Seq. ID No. 1623, Seq. ID No. 1625, Seq. ID No. 1627, Seq. ID No. 1629, Seq. ID No. 1631, Seq. ID No. 1633, Seq. ID No. 1639, Seq. ID No. 1643, Seq. ID No. 1655, Seq. ID No. 1667, Seq. ID No. 1671, Seq. ID No. 1693, Seq. ID No. 1695, Seq. ID No. 1701, Seq. ID No. 1703, Seq. ID No. 1705, Seq. ID No. 1721, Seq. ID No. 1723, Seq. ID No. 1731, Seq. ID No. 1737,.Seq. ID No. 1743, Seq. ID No. 1759, Seq. ID No. 1763, Seq. ID No. 1773, Seq. ID No. 1775, Seq. ID No. 1777, Seq. ID No. 1779, Seq. ID No. 1781, Seq. ID No. 1799, Seq. ID No. 1801, Seq. ID No. 1803, Seq. ID No. 1807, Seq. ID No. 1823, Seq. ID No. 1831, Seq. ID No. 1851, Seq. ID No. 1857, Seq. ID No. 1875, Seq. ID No. 1899, Seq. ID No. 1915, Seq. ID No. 1927, Seq. ID No. 1929, Seq. ID No. 1973, Seq. ID No. 1977, Seq. ID No. 1981, Seq. ID No. 2005, Seq. ID No. 2007, Seq. ID No. 2009, Seq. ID No. 2011, Seq. ID No. 2017, Seq. ID No. 2019, Seq. ID No. 2023, Seq. ID No. 2025, Seq. ID No. 2027, Seq. ID No. 2039, Seq. ID No. 2041, Seq. ID No. 2043, Seq. ID No. 2045, Seq. ID No. 2049, Seq. ID No. 2081, Seq. ID No. 2083, Seq. ID No. 2085, Seq. ID No. 2105, Seq. ID No. 2107, Seq. ID No. 2109, Seq. ID No. 2111, Seq. ID No. 2129, Seq. ID No. 2131, Seq. ID No. 2133, Seq. ID No. 2145, Seq. ID No. 2155, Seq. ID No. 2157, Seq. ID No. 2159, Seq. ID No. 2161, Seq. ID No. 2171, Seq. ID No. 2187, Seq. ID No. 2189, Seq. ID No. 2201, Seq. ID No. 2203, Seq. ID No. 2209, Seq. ID No. 2211, Seq. ID No. 2219, Seq. ID No. 2225, Seq. ID No. 2243, Seq. ID No. 2245, Seq. ID No. 2247, Seq. ID No. 2249, Seq. ID No. 2251, Seq. ID No. 2255, Seq. ID No. 2277, Seq. ID No. 2279, Seq. ID No. 2341, Seq. ID No. 2351, Seq. ID No. 2353, Seq. ID No. 2355, Seq. ID No. 2357, Seq. ID No. 2359, Seq. ID No. 2365, Seq. ID No. 2367, Seq. ID No. 2371, Seq. ID No. 2375, Seq. ID No. 2393, Seq. ID No. 2395, Seq. ID No. 2425, Seq. ID No. 2427, Seq. ID No. 2431, Seq. ID No. 2439, Seq. ID No. 2441, Seq. ID No. 2443, Seq. ID No. 2447, Seq. ID No. 2459, Seq. ID No. 2479, Seq. ID No. 2493, Seq. ID No. 2501, Seq. ID No. 2503, Seq. ID No. 2521, Seq. ID No. 2523, Seq. ID No. 2527, Seq. ID No. 2591, Seq. ID No. 2601, Seq. ID No. 2603, Seq. ID No. 2611, Seq. ID No. 2627, Seq. ID No. 2629, Seq. ID No. 2653, Seq. ID No. 2661, Seq. ID No. 2691, Seq. ID No. 2695, Seq. ID No. 2697, Seq. ID No. 2717, Seq. ID No. 2729, Seq. ID No. 2731, Seq. ID No. 2733, Seq. ID No. 2735, Seq. ID No. 2739, Seq. ID No. 2741, Seq. ID No. 2743, Seq. ID No. 2777, Seq. ID No. 2779, Seq. ID No. 2781, Seq. ID No. 2783, Seq. ID No. 2801, Seq. ID No. 2805, Seq. ID No. 2807, Seq. ID No. 2809, Seq. ID No. 2811, Seq. ID No. 2813, Seq. ID No. 2815, Seq. ID No. 2817, Seq. ID No. 2819, Seq. ID No. 2829, Seq. ID No. 2831, Seq. ID No. 2833, Seq. ID No. 2835, Seq. ID No. 2837, Seq. ID No. 2839, Seq. ID No. 2841, Seq. ID No. 2845, Seq. ID No. 2849, Seq. ID No. 2851, Seq. ID No. 2853, Seq. ID No. 2855, Seq. ID No. 2857, Seq. ID No. 2859, Seq. ID No. 2861, Seq. ID No. 2865, Seq. ID No. 2873, Seq. ID No. 2875, Seq. ID No. 2877, Seq. ID No. 2883, Seq. ID No. 2885, Seq. ID No. 2887, Seq. ID No. 2889, Seq. ID No. 2893, Seq. ID No. 2895, Seq. ID No. 2903, Seq. ID No. 2909, Seq. ID No. 2935, Seq. ID No. 2937, Seq. ID No. 2939, Seq. ID No. 2941, Seq. ID No. 2943, Seq. ID No. 2945, Seq. ID No. 2947, Seq. ID No. 2949, Seq. ID No. 2951, Seq. ID No. 2953, Seq. ID No. 2965, Seq. ID No. 2967, Seq. ID No. 2973, Seq. ID No. 2975, Seq. ID No. 2977, Seq. ID No. 2981, Seq. ID No. 2983, Seq. ID No. 2985, Seq. ID No. 2987, Seq. ID No. 2989, Seq. ID No. 2991, Seq. ID No. 3001, Seq. ID No. 3007, Seq. ID No. 3009, Seq. ID No. 3011, Seq. ID No. 3021, Seq. ID No. 3023, Seq. ID No. 3041, Seq. ID No. 3045, Seq. ID No. 3071, Seq. ID No. 3087, Seq. ID No. 3105, Seq. ID No. 3107, Seq. ID No. 3119, Seq. ID No. 3121, Seq. ID No. 3123, Seq. ID No. 3151, Seq. ID No. 3153, Seq. ID No. 3167, Seq. ID No. 3173, Seq. ID No. 3175, Seq. ID No. 3177, Seq. ID No. 3181, Seq. ID No. 3193, Seq. ID No. 3213, Seq. ID No. 3215, Seq. ID No. 3217, Seq. ID No. 3219, Seq. ID No. 3221, Seq. ID No. 3223, Seq. ID No. 3231, Seq. ID No. 3233, Seq. ID No. 3235, Seq. ID No. 3237, Seq. ID No. 3239, Seq. ID No. 3241, Seq. ID No. 3243, Seq. ID No. 3245, Seq. ID No. 3247, Seq. ID No. 3249, Seq. ID No. 3261, Seq. ID No. 3263, Seq. ID No. 3265, Seq. ID No. 3267, Seq. ID No. 3269, Seq. ID No. 3271, Seq. ID No. 3273, Seq. ID No. 3275, Seq. ID No. 3277, Seq. ID No. 3325, Seq. ID No. 3331, Seq. ID No. 3339, Seq. ID No. 3341, Seq. ID No. 3349, Seq. ID No. 3361, Seq. ID No. 3363, Seq. ID No. 3369, Seq. ID No. 3371, Seq. ID No. 3389, Seq. ID No. 3403, Seq. ID No. 3417, Seq. ID No. 3419, Seq. ID No. 3421, Seq. ID No. 3433, Seq. ID No. 3437, Seq. ID No. 3441, Seq. ID No. 3443, Seq. ID No. 3445, Seq. ID No. 3447, Seq. ID No. 3473, Seq. ID No. 3481, Seq. ID No. 3483, Seq. ID No. 3489, Seq. ID No. 3503, Seq. ID No. 3505, Seq. ID No. 3509, Seq. ID No. 3511, Seq. ID No. 3523, Seq. ID No. 3525, Seq. ID No. 3531, Seq. ID No. 3533, Seq. ID No. 3539, Seq. ID No. 3541, Seq. ID No. 3543, Seq. ID No. 3545, Seq. ID No. 3547, Seq. ID No. 3555, Seq. ID No. 3557, Seq. ID No. 3563, Seq. ID No. 3565, Seq. ID No. 3571, Seq. ID No. 3573, Seq. ID No. 3581, Seq. ID No. 3587, Seq. ID No. 3591, Seq. ID No. 3593, Seq. ID No. 3595, Seq. ID No. 3611, Seq. ID No. 3613, Seq. ID No. 3617, Seq. ID No. 3619, Seq. ID No. 3621, Seq. ID No. 3623, Seq. ID No. 3625, Seq. ID No. 3631, Seq. ID No. 3637, Seq. ID No. 3639, Seq. ID No. 3655, Seq. ID No. 3657, Seq. ID No. 3659, Seq. ID No. 3661, Seq. ID No. 3677, Seq. ID No. 3679, Seq. ID No. 3683, Seq. ID No. 3685, Seq. ID No. 3701, Seq. ID No. 3703, Seq. ID No. 3717, Seq. ID No. 3719, Seq. ID No. 3721, Seq. ID No. 3723, Seq. ID No. 3729, Seq. ID No. 3731, Seq. ID No. 3733, Seq. ID No. 3735, Seq. ID No. 3737, Seq. ID No. 3739, Seq. ID No. 3741, Seq. ID No. 3749, Seq. ID No. 3751, Seq. ID No. 3753, Seq. ID No. 3755, Seq. ID No. 3761, Seq. ID No. 3781, Seq. ID No. 3783, Seq. ID No. 3785, Seq. ID No. 3787, Seq. ID No. 3789, Seq. ID No. 3791, Seq. ID No. 3799, Seq. ID No. 3807, Seq. ID No. 3809, Seq. ID No. 3811, Se4. ID No. 3813, Seq. ID No. 3815, Seq. ID No. 3817, Seq. ID No. 3827, Seq. ID No. 3829, Seq. ID No. 3831, Seq. ID No. 3833, Seq. ID No. 3835, Seq. ID No. 3849, Seq. ID No. 3879, Seq. ID No. 3881, Seq. ID No. 3895, Seq. ID No. 3903, Seq. ID No. 3909, Seq. ID No. 3915, Seq. ID No. 3939, Seq. ID No. 3959, Seq. ID No. 3961, Seq. ID No. 3963, Seq. ID No. 3965, Seq. ID No. 3973, Seq. ID No. 3977, Seq. ID No. 3979, Seq. ID No. 3993, Seq. ID No. 3995, Seq. ID No. 3997, Seq. ID No. 4005, Seq. ID No. 4007, Seq. ID No. 4009, Seq. ID No. 4041, Seq. ID No. 4043, Seq. ID No. 4045, Seq. ID No. 4069, Seq. ID No. 4077, Seq. ID No. 4097, Seq. ID No. 4113, Seq. ID No. 4123, Seq. ID No. 4131, Seq. ID No. 4133, Seq. ID No. 4135, Seq. ID No. 4139, Seq. ID No. 4151, Seq. ID No. 4153, Seq. ID No. 4155, Seq. ID No. 4181, Seq. ID No. 4221, Seq. ID No. 4241, Seq. ID No. 4253, Seq. ID No. 4257, Seq. ID No. 4259, Seq. ID No. 4261, Seq. ID No. 4263, Seq. ID No. 4265, Seq. ID No. 4269, Seq. ID No. 4275, Seq. ID No. 4277, Seq. ID No. 4279, Seq. ID No. 4281, Seq. ID No. 4285, Seq. ID No. 4287, Seq. ID No. 4297, Seq. ID No. 4305, Seq. ID No. 4307, Seq. ID No. 4363, Seq. ID No. 4365, Seq. ID No. 4371, Seq. ID No. 4373, Seq. ID No. 4395, Seq. ID No. 4399, Seq. ID No. 4403, Seq. ID No. 4425, Seq. ID No. 4437, Seq. ID No. 4439, Seq. ID No. 4441, Seq. ID No. 4453, Seq. ID No. 4455, Seq. ID No. 4513, Seq. ID No. 4515, Seq. ID No. 4551, Seq. ID No. 4561, Seq. ID No. 4573, Seq. ID No. 4575, Seq. ID No. 4587, Seq. ID No. 4589, Seq. ID No. 4591, Seq. ID No. 4593, Seq. ID No. 4595, Seq. ID No. 4617, Seq. ID No. 4619, Seq. ID No. 4621, Seq. ID No. 4625, Seq. ID No. 4627, Seq. ID No. 4633, Seq. ID No. 4635, Seq. ID No. 4637, Seq. ID No. 4639, Seq. ID No. 4659, Seq. ID No. 4681, Seq. ID No. 4687, Seq. ID No. 4695, Seq. ID No. 4699, Seq. ID No. 4701, Seq. ID No. 4721, Seq. ID No. 4735, Seq. ID No. 4737, Seq. ID No. 4741, Seq. ID No. 4783, Seq. ID No. 4789, Seq. ID No. 4805, Seq. ID No. 4811, Seq. ID No. 4817, Seq. ID No. 4819, Seq. ID No. 4821, Seq. ID No. 4823, Seq. ID No. 4835, Seq. ID No. 4853, Seq. ID No. 4885, Seq. ID No. 4913, Seq. ID No. 4923, Seq. ID No. 4949, Seq. ID No. 4951, Seq. ID No. 4975, Seq. ID No. 4981, Seq. ID No. 4983, Seq. ID No. 4987, Seq. ID No. 4993, Seq. ID No. 4999, Seq. ID No. 5001, Seq. ID No. 5011, Seq. ID No. 5013, Seq. ID No. 5023, Seq. ID No. 5031, Seq. ID No. 5033, Seq. ID No. 5035, Seq. ID No. 5037, Seq. ID No. 5041, Seq. ID No. 5043, Seq. ID No. 5053, Seq. ID No. 5061, Seq. ID No. 5063, Seq. ID No. 5085, Seq. ID No. 5093, Seq. ID No. 5151, Seq. ID No. 5153, Seq. ID No. 5159, Seq. ID No. 5161, Seq. ID No. 5167, Seq. ID No. 5173, Seq. ID No. 5175, Seq. ID No. 5177, Seq. ID No. 5189, Seq. ID No. 5191, Seq. ID No. 5193, Seq. ID No. 5197, Seq. ID No. 5199, Seq. ID No. 5221, Seq. ID No. 5223, Seq. ID No. 5225, Seq. ID No. 5227, Seq. ID No. 5237, Seq. ID No. 5267, Seq. ID No. 5269, Seq. ID No. 5271, Seq. ID No. 5281, Seq. ID No. 5301, Seq. ID No. 5303, Seq. ID No. 5315, Seq. ID No. 5321, Seq. ID No. 5327, Seq. ID No. 5347, Seq. ID No. 5385, Seq. ID No. 5445, Seq. ID No. 5447, Seq. ID No. 5459, Seq. ID No. 5465, Seq. ID No. 5467, Seq. ID No. 5479, Seq. ID No. 5481, Seq. ID No. 5485, Seq. ID No. 5487, Seq. ID No. 5515, Seq. ID No. 5517, Seq. ID No. 5533, Seq. ID No. 5543, Seq. ID No. 5545, Seq. ID No. 5549, Seq. ID No. 5569, Seq. ID No. 5577, Seq. ID No. 5593, Seq. ID No. 5595, Seq. ID No. 5609, Seq. ID No. 5623, Seq. ID No. 5625, Seq. ID No. 5639, Seq. ID No. 5641, Seq. ID No. 5643, Seq. ID No. 5649, Seq. ID No. 5659, Seq. ID No. 5661, Seq. ID No. 5663, Seq. ID No. 5667, Seq. ID No. 5671, Seq. ID No. 5687, Seq. ID No. 5689, Seq. ID No. 5703, Seq. ID No. 5705, Seq. ID No. 5709, Seq. ID No. 5711, Seq. ID No. 5737, Seq. ID No. 5739, Seq. ID No. 5745, Seq. ID No. 5751, Seq. ID No. 5763, Seq. ID No. 5797, Seq. ID No. 5811, Seq. ID No. 5813, Seq. ID No. 5815, Seq. ID No. 5827, Seq. ID No. 5829, Seq. ID No. 5843, Seq. ID No. 5845, Seq. ID No. 5873, Seq. ID No. 5879, Seq. ID No. 5883, Seq. ID No. 5885, Seq. ID No. 5887, Seq. ID No. 5889, Seq. ID No. 5891, Seq. ID No. 5931, Seq. ID No. 5955, Seq. ID No. 5963, Seq. ID No. 5965, Seq. ID No. 5975, Seq. ID No. 5983, Seq. ID No. 5985, Seq. ID No. 5987, Seq. ID No. 5989, Seq. ID No. 6005, Seq. ID No. 6013, Seq. ID No. 6031, Seq. ID No. 6035, Seq. ID No. 6045, Seq. ID No. 6059, Seq. ID No. 6061, Seq. ID No. 6063, Seq. ID No. 6073, Seq. ID No. 6081, Seq. ID No. 6091, Seq. ID No. 6093, Seq. ID No. 6127, Seq. ID No. 6159, Seq. ID No. 6197, Seq. ID No. 6199, Seq. ID No. 6203, Seq. ID No. 6217, Seq. ID No. 6221, Seq. ID No. 6223, Seq. ID No. 6237, Seq. ID No. 6239, Seq. ID No. 6241, Seq. ID No. 6245, Seq. ID No. 6247, Seq. ID No. 6249, Seq. ID No. 6259, Seq. ID No. 6265, Seq. ID No. 6267, Seq. ID No. 6273, Seq. ID No. 6283, Seq. ID No. 6285, Seq. ID No. 6323, Seq. ID No. 6325, Seq. ID No. 6327, Seq. ID No. 6361, Seq. ID No. 6363, Seq. ID No. 6377, Seq. ID No. 6381, Seq. ID No. 6401, Seq. ID No. 6403, Seq. ID No. 6419, Seq. ID No. 6425, Seq. ID No. 6427, Seq. ID No. 6429, Seq. ID No. 6431, Seq. ID No. 6433, Seq. ID No. 6435, Seq. ID No. 6437, Seq. ID No. 6439, Seq. ID No. 6447, Seq. ID No. 6449, Seq. ID No. 6451, Seq. ID No. 6453, Seq. ID No. 6455, Seq. ID No. 6457, Seq. ID No. 6465, Seq. ID No. 6467, Seq. ID No. 6469, Seq. ID No. 6471, Seq. ID No. 6473, Seq. ID No. 6475, Seq. ID No. 6477, Seq. ID No. 6479, Seq. ID No. 6481, Seq. ID No. 6483, Seq. ID No. 6485, Seq. ID No. 6487, Seq. ID No. 6489, Seq. ID No. 6491, Seq. ID No. 6495, Seq. ID No. 6497, Seq. ID No. 6507, Seq. ID No. 6517, Seq. ID No. 6519, Seq. ID No. 6521, Seq. ID No. 6523, Seq. ID No. 6525, Seq. ID No. 6527, Seq. ID No. 6529, Seq. ID No. 6533, Seq. ID No. 6539, Seq. ID No. 6541, Seq. ID No. 6543, Seq. ID No. 6555, Seq. ID No. 6557, Seq. ID No. 6561, Seq. ID No. 6563, Seq. ID No. 6565, Seq. ID No. 6567, Seq. ID No. 6569, Seq. ID No. 6571, Seq. ID No. 6573, Seq. ID No. 6581, Seq. ID No. 6583, Seq. ID No. 6631, Seq. ID No. 6635, Seq. ID No. 6637, Seq. ID No. 6639, Seq. ID No. 6643, Seq. ID No. 6645, Seq. ID No. 6647, and Seq. ID No. 6649.

In yet another preferred embodiment, the polynucleotide is selected from the Alloiococcus otitidis polynucleotides, which encode polypeptides, identified by Glimmer™ ORF finder program, and the polynucleotide is selected from one of Seq. ID No. 1, Seq. ID No. 5, Seq. ID No. 11, Seq. ID No. 13, Seq. ID No. 15, Seq. ID No. 17, Seq. ID No. 25, Seq. ID No. 27, Seq. ID No. 29, Seq. ID No. 35, Seq. ID No. 37, Seq. ID No. 41, Seq. ID No. 43, Seq. ID No. 47, Seq. ID No. 51, Seq. ID No. 53, Seq. ID No. 59, Seq. ID No. 63, Seq. ID No. 69, Seq. ID No. 73, Seq. ID No. 75, Seq. ID No. 81, Seq. ID No. 83, Seq. ID No. 89, Seq. ID No. 93, Seq. ID No. 97, Seq. ID No. 103, Seq. ID No. 111, Seq. ID No. 121, Seq. ID No. 125, Seq. ID No. 129, Seq. ID No. 135, Seq. ID No. 137, Seq. ID No. 145, Seq. ID No. 149, Seq. ID No. 157, Seq. ID No. 167, Seq. ID No. 173, Seq. ID No. 183, Seq. ID No. 187, Seq. ID No. 189, Seq. ID No. 191, Seq. ID No. 195, Seq. ID No. 197, Seq. ID No. 203, Seq. ID No. 207, Seq. ID No. 215, Seq. ID No. 219, Seq. ID No. 223, Seq. ID No. 225, Seq. ID No. 227, Seq. ID No. 231, Seq. ID No. 233, Seq. ID No. 239, Seq. ID No. 241, Seq. ID No. 247, Seq. ID No. 253, Seq. ID No. 261, Seq. ID No. 265, Seq. ID No. 269, Seq. ID No. 273, Seq. ID No. 275, Seq. ID No. 277, Seq. ID No. 283, Seq. ID No. 291, Seq. ID No. 293, Seq. ID No. 297, Seq. ID No. 301, Seq. ID No. 307, Seq. ID No. 309, Seq. ID No. 313, Seq. ID No. 317, Seq. ID No. 321, Seq. ID No. 325, Seq. ID No. 335, Seq. ID No. 343, Seq. ID No. 345, Seq. ID No. 353, Seq. ID No. 355, Seq. ID No. 361, Seq. ID No. 363, Seq. ID No. 367, Seq. ID No. 375, Seq. ID No. 379, Seq. ID No. 383, Seq. ID No. 389, Seq. ID No. 391, Seq. ID No. 397, Seq. ID No. 405, Seq. ID No. 411, Seq. ID No. 415, Seq. ID No. 417, Seq. ID No. 423, Seq. ID No. 425, Seq. ID No. 427, Seq. ID No. 429, Seq. ID No. 435, Seq. ID No. 437, Seq. ID No. 439, Seq. ID No. 441, Seq. ID No. 445, Seq. ID No. 447, Seq. ID No. 453, Seq. ID No. 461, Seq. ID No. 463, Seq. ID No. 471, Seq. ID No. 485, Seq. ID No. 501, Seq. ID No. 505, Seq. ID No. 511, Seq. ID No. 515, Seq. ID No. 517, Seq. ID No. 521, Seq. ID No. 523, Seq. ID No. 531, Seq. ID No. 533, Seq. ID No. 539, Seq. ID No. 541, Seq. ID No. 543, Seq. ID No. 551, Seq. ID No. 555, Seq. ID No. 561, Seq. ID No. 565, Seq. ID No. 569, Seq. ID No. 575, Seq. ID No. 579, Seq. ID No. 587, Seq. ID No. 593, Seq. ID No. 597, Seq. ID No. 603, Seq. ID No. 605, Seq. ID No. 609, Seq. ID No. 611, Seq. ID No. 613, Seq. ID No. 615, Seq. ID No. 623, Seq. ID No. 627, Seq. ID No. 629, Seq. ID No. 631, Seq. ID No. 635, Seq. ID No. 637, Seq. ID No. 647, Seq. ID No. 649, Seq. ID No. 661, Seq. ID No. 669, Seq. ID No. 673, Seq. ID No. 675, Seq. ID No. 681, Seq. ID No. 683, Seq. ID No. 689, Seq. ID No. 701, Seq. ID No. 703, Seq. ID No. 709, Seq. ID No. 713, Seq. ID No. 729, Seq. ID No. 733, Seq. ID No. 735, Seq. ID No. 737, Seq. ID No. 741, Seq. ID No. 747, Seq. ID No. 751, Seq. ID No. 753, Seq. ID No. 759, Seq. ID No. 765, Seq. ID No. 767, Seq. ID No. 771, Seq. ID No. 781, Seq. ID No. 783, Seq. ID No. 787, Seq. ID No. 791, Seq. ID No. 795, Seq. ID No. 799, Seq. ID No. 807, Seq. ID No. 817, Seq. ID No. 819, Seq. ID No. 823, Seq. ID No. 827, Seq. ID No. 831, Seq. ID No. 835, Seq. ID No. 837, Seq. ID No. 839, Seq. ID No. 847, Seq. ID No. 851, Seq. ID No. 855, Seq. ID No. 859, Seq. ID No. 867, Seq. ID No. 871, Seq. ID No. 875, Seq. ID No. 881, Seq. ID No. 885, Seq. ID No. 889, Seq. ID No. 891, Seq. ID No. 895, Seq. ID No. 901, Seq. ID No. 907, Seq. ID No. 915, Seq. ID No. 919, Seq. ID No. 925, Seq. ID No. 929, Seq. ID No. 935, Seq. ID No. 939, Seq. ID No. 949, Seq. ID No. 951, Seq. ID No. 955, Seq. ID No. 961, Seq. ID No. 965, Seq. ID No. 967, Seq. ID No. 971, Seq. ID No. 985, Seq. ID No. 993, Seq. ID No. 997, Seq. ID No. 1003, Seq. ID No. 1005, Seq. ID No. 1009, Seq. ID No. 1017, Seq. ID No. 1023, Seq. ID No. 1039, Seq. ID No. 1041, Seq. ID No. 1043, Seq. ID No. 1051, Seq. ID No. 1053, Seq. ID No. 1057, Seq. ID No. 1061, Seq. ID No. 1065, Seq. ID No. 1067, Seq. ID No. 1069, Seq. ID No. 1073, Seq. ID No. 1075, Seq. ID No. 1083, Seq. ID No. 1087, Seq. ID No. 1091, Seq. ID No. 1093, Seq. ID No. 1097, Seq. ID No. 1099, Seq. ID No. 1103, Seq. ID No. 1109, Seq. ID No. 1115, Seq. ID No. 1117, Seq. ID No. 1123, Seq. ID No. 1129, Seq. ID No. 1133, Seq. ID No. 1137, Seq. ID No. 1143, Seq. ID No. 1147, Seq. ID No. 1149, Seq. ID No. 1155, Seq. ID No. 1157, Seq. ID No. 1159, Seq. ID No. 1163, Seq. ID No. 1171, Seq. ID No. 1179, Seq. ID No. 1181, Seq. ID No. 1187, Seq. ID No. 1193, Seq. ID No. 1199, Seq. ID No. 1205, Seq. ID No. 1211, Seq. ID No. 1217, Seq. ID No. 1221, Seq. ID No. 1223, Seq. ID No. 1225, Seq. ID No. 1231, Seq. ID No. 1233, Seq. ID No. 1237, Seq. ID No. 1241, Seq. ID No. 1253, Seq. ID No. 1257, Seq. ID No. 1263, Seq. ID No. 1271, Seq. ID No. 1281, Seq. ID No. 1317, Seq. ID No. 1323, Seq. ID No. 1331, Seq. ID No. 1335, Seq. ID No. 1339, Seq. ID No. 1345, Seq. ID No. 1347, Seq. ID No. 1361, Seq. ID No. 1369, Seq. ID No. 1377, Seq. ID No. 1379, Seq. ID No. 1387, Seq. ID No. 1393, Seq. ID No. 1395, Seq. ID No. 1399, Seq. ID No. 1403, Seq. ID No. 1405, Seq. ID No. 1411, Seq. ID No. 1413, Seq. ID No. 1415, Seq. ID No. 1419, Seq. ID No. 1425, Seq. ID No. 1427, Seq. ID No. 1429, Seq. ID No. 1431, Seq. ID No. 1433, Seq. ID No. 1437, Seq. ID No. 1443, Seq. ID No. 1449, Seq. ID No. 1451, Seq. ID No. 1455, Seq. ID No. 1457, Seq. ID No. 1459, Seq. ID No. 1463, Seq. ID No. 1465, Seq. ID No. 1469, Seq. ID No. 1471, Seq. ID No. 1473, Seq. ID No. 1475, Seq. ID No. 1477, Seq. ID No. 1485, Seq. ID No. 1487, Seq. ID No. 1491, Seq. ID No. 1493, Seq. ID No. 1495, Seq. ID No. 1497, Seq. ID No. 1501, Seq. ID No. 1505, Seq. ID No. 1507, Seq. ID No. 1509, Seq. ID No. 1517, Seq. ID No. 1525, Seq. ID No. 1527, Seq. ID No. 1531, Seq. ID No. 1539, Seq. ID No. 1541, Seq. ID No. 1543, Seq. ID No. 1549, Seq. ID No. 1561, Seq. ID No. 1563, Seq. ID No. 1567, Seq. ID No. 1571, Seq. ID No. 1573, Seq. ID No. 1577, Seq. ID No. 1581, Seq. ID No. 1589, Seq. ID No. 1593, Seq. ID No. 1595, Seq. ID No. 1597, Seq. ID No. 1603, Seq. ID No. 1607, Seq. ID No. 1611, Seq. ID No. 1613, Seq. ID No. 1619, Seq. ID No. 1623, Seq. ID No. 1627, Seq. ID No. 1631, Seq. ID No. 1637, Seq. ID No. 1639, Seq. ID No. 1641, Seq. ID No. 1647, Seq. ID No. 1653, Seq. ID No. 1657, Seq. ID No. 1665, Seq. ID No. 1669, Seq. ID No. 1681, Seq. ID No. 1685, Seq. ID No. 1689, Seq. ID No. 1691, Seq. ID No. 1697, Seq. ID No. 1699, Seq. ID No. 1707, Seq. ID No. 1711, Seq. ID No. 1713, Seq. ID No. 1719, Seq. ID No. 1723, Seq. ID No. 1729, Seq. ID No. 1735, Seq. ID No. 1737, Seq. ID No. 1741, Seq. ID No. 1747, Seq. ID No. 1751, Seq. ID No. 1757, Seq. ID No. 1759, Seq. ID No. 1761, Seq. ID No. 1771, Seq. ID No. 1777, Seq. ID No. 1785, Seq. ID No. 1789, Seq. ID No. 1791, Seq. ID No. 1797, Seq. ID No. 1801, Seq. ID No. 1803, Seq. ID No. 1805, Seq. ID No. 1811, Seq. ID No. 1817, Seq. ID No. 1819, Seq. ID No. 1821, Seq. ID No. 1825, Seq. ID No. 1829, Seq. ID No. 1837, Seq. ID No. 1843, Seq. ID No. 1845, Seq. ID No. 1847, Seq. ID No. 1849, Seq. ID No. 1851, Seq. ID No. 1855, Seq. ID No. 1859, Seq. ID No. 1863, Seq. ID No. 1865, Seq. ID No. 1867, Seq. ID No. 1871, Seq. ID No. 1873, Seq. ID No. 1877, Seq. ID No. 1885, Seq. ID No. 1887, Seq. ID No. 1889, Seq. ID No. 1895, Seq. ID No. 1897, Seq. ID No. 1899, Seq. ID No. 1903, Seq. ID No. 1905, Seq. ID No. 1913, Seq. ID No. 1919, Seq. ID No. 1923, Seq. ID No. 1925, Seq. ID No. 1931, Seq. ID No. 1935, Seq. ID No. 1937, Seq. ID No. 1943, Seq. ID No. 1945, Seq. ID No. 1949, Seq. ID No. 1953, Seq. ID No. 1957, Seq. ID No. 1959, Seq. ID No. 1961, Seq. ID No. 1965, Seq. ID No. 1967, Seq. ID No. 1971, Seq. ID No. 1975, Seq. ID No. 1979, Seq. ID No. 1983, Seq. ID No. 1987, Seq. ID No. 1989, Seq. ID No. 1991, Seq. ID No. 1999, Seq. ID No. 2001, Seq. ID No. 2003, Seq. ID No. 2009, Seq. ID No. 2015, Seq. ID No. 2019, Seq. ID No. 2021, Seq. ID No. 2031, Seq. ID No. 2035, Seq. ID No. 2037, Seq. ID No. 2045, Seq. ID No. 2047, Seq. ID No. 2055, Seq. ID No. 2059, Seq. ID No. 2065, Seq. ID No. 2067, Seq. ID No. 2069, Seq. ID No. 2071, Seq. ID No. 2073, Seq. ID No. 2079, Seq. ID No. 2091, Seq. ID No. 2093, Seq. ID No. 2097, Seq. ID No. 2099, Seq. ID No. 2103, Seq. ID No. 2115, Seq. ID No. 2119, Seq. ID No. 2127, Seq. ID No. 2135, Seq. ID No. 2137, Seq. ID No. 2141, Seq. ID No. 2143, Seq. ID No. 2147, Seq. ID No. 2153, Seq. ID No. 2159, Seq. ID No. 2161, Seq. ID No. 2165, Seq. ID No. 2169, Seq. ID No. 2179, Seq. ID No. 2183, Seq. ID No. 2185, Seq. ID No. 2189, Seq. ID No. 2191, Seq. ID No. 2193, Seq. ID No. 2195, Seq. ID No. 2199, Seq. ID No. 2207, Seq. ID No. 2217, Seq. ID No. 2219, Seq. ID No. 2221, Seq. ID No. 2223, Seq. ID No. 2227, Seq. ID No. 2229, Seq. ID No. 2231, Seq. ID No. 2233, Seq. ID No. 2241, Seq. ID No. 2245, Seq. ID No. 2251, Seq. ID No. 2253, Seq. ID No. 2257, Seq. ID No. 2259, Seq. ID No. 2263, Seq. ID No. 2267, Seq. ID No. 2269, Seq. ID No. 2275, Seq. ID No. 2281, Seq. ID No. 2287, Seq. ID No. 2297, Seq. ID No. 2299, Seq. ID No. 2301, Seq. ID No. 2315, Seq. ID No. 2329, Seq. ID No. 2333, Seq. ID No. 2335, Seq. ID No. 2339, Seq. ID No. 2343, Seq. ID No. 2345, Seq. ID No. 2349, Seq. ID No. 2351, Seq. ID No. 2357, Seq. ID No. 2359, Seq. ID No. 2363, Seq. ID No. 2365, Seq. ID No. 2369, Seq. ID No. 2373, Seq. ID No. 2381, Seq. ID No. 2391, Seq. ID No. 2399, Seq. ID No. 2401, Seq. ID No. 2405, Seq. ID No. 2409, Seq. ID No. 2415, Seq. ID No. 2417, Seq. ID No. 2421, Seq. ID No. 2423, Seq. ID No. 2427, Seq. ID No. 2429, Seq. ID No. 2431, Seq. ID No. 2435, Seq. ID No. 2437, Seq. ID No. 2439, Seq. ID No. 2443, Seq. ID No. 2445, Seq. ID No. 2449, Seq. ID No. 2451, Seq. ID No. 2453, Seq. ID No. 2457, Seq. ID No. 2463, Seq. ID No. 2465, Seq. ID No. 2469, Seq. ID No. 2471, Seq. ID No. 2475, Seq. ID No. 2477, Seq. ID No. 2483, Seq. ID No. 2485, Seq. ID No. 2491, Seq. ID No. 2495, Seq. ID No. 2505, Seq. ID No. 2507, Seq. ID No. 2513, Seq. ID No. 2519, Seq. ID No. 2525, Seq. ID No. 2529, Seq. ID No. 2535, Seq. ID No. 2537, Seq. ID No. 2539, Seq. ID No. 2549, Seq. ID No. 2553, Seq. ID No. 2563, Seq. ID No. 2567, Seq. ID No. 2575, Seq. ID No. 2577, Seq. ID No. 2581, Seq. ID No. 2589, Seq. ID No. 2593, Seq. ID No. 2595, Seq. ID No. 2599, Seq. ID No. 2603, Seq. ID No. 2607, Seq. ID No. 2609, Seq. ID No. 2613, Seq. ID No. 2617, Seq. ID No. 2619, Seq. ID No. 2623, Seq. ID No. 2625, Seq. ID No. 2631, Seq. ID No. 2633, Seq. ID No. 2639, Seq. ID No. 2641, Seq. ID No. 2645, Seq. ID No. 2649, Seq. ID No. 2651, Seq. ID No. 2655, Seq. ID No. 2659, Seq. ID No. 2663, Seq. ID No. 2667, Seq. ID No. 2669, Seq. ID No. 2671, Seq. ID No. 2677, Seq. ID No. 26$7, Seq. ID No. 2689, Seq. ID No. 2693, Seq. ID No. 2699, Seq. ID No. 2703, Seq. ID No. 2709, Seq. ID No. 2711, Seq. ID No. 2715, Seq. ID No. 2719, Seq. ID No. 2725, Seq. ID No. 2727, Seq. ID No. 2737, Seq. ID No. 2743, Seq. ID No. 2747, Seq. ID No. 2751, Seq. ID No. 2753, Seq. ID No. 2759, Seq. ID No. 2763, Seq. ID No. 2765, Seq. ID No. 2767, Seq. ID No. 2771, Seq. ID No. 2773, Seq. ID No. 2775, Seq. ID No. 2781, Seq. ID No. 2787, Seq. ID No. 2789, Seq. ID No. 2791, Seq. ID No. 2793, Seq. ID No. 2797, Seq. ID No. 2799, Seq. ID No. 2803, Seq. ID No. 2805, Seq. ID No. 2811, Seq. ID No. 2815, Seq. ID No. 2823, Seq. ID No. 2827, Seq. ID No. 2843, Seq. ID No. 2847, Seq. ID No. 2863, Seq. ID No. 2867, Seq. ID No. 2869, Seq. ID No. 2871, Seq. ID No. 2877, Seq. ID No. 2881, Seq. ID No. 2891, Seq. ID No. 2899, Seq. ID No. 2901, Seq. ID No. 2903, Seq. ID No. 2907, Seq. ID No. 2911, Seq. ID No. 2913, Seq. ID No. 2917, Seq. ID No. 2919, Seq. ID No. 2921, Seq. ID No. 2923, Seq. ID No. 2927, Seq. ID No. 2933, Seq. ID No. 2945, Seq. ID No. 2953, Seq. ID No. 2957, Seq. ID No. 2963, Seq. ID No. 2971, Seq. ID No. 2973, Seq. ID No. 2977, Seq. ID No. 2979, Seq. ID No. 2983, Seq. ID No. 2985, Seq. ID No. 2991, Seq. ID No. 2993, Seq. ID No. 2999, Seq. ID No. 3001, Seq. ID No. 3005, Seq. ID No. 3011, Seq. ID No. 3019, Seq. ID No. 3027, Seq. ID No. 3033, Seq. ID No. 3035, Seq. ID No. 3039, Seq. ID No. 3043, Seq. ID No. 3045, Seq. ID No. 3049, Seq. ID No. 3055, Seq. ID No. 3059, Seq. ID No. 3061, Seq. ID No. 3067, Seq. ID No. 3069, Seq. ID No. 3081, Seq. ID No. 3089, Seq. ID No. 3091, Seq. ID No. 3093, Seq. ID No. 3097, Seq. ID No. 3103, Seq. ID No. 3107, Seq. ID No. 3111, Seq. ID No. 3117, Seq. ID No. 3125, Seq. ID No. 3133, Seq. ID No. 3135, Seq. ID No. 3137, Seq. ID No. 3141, Seq. ID No. 3143, Seq. ID No. 3149, Seq. ID No. 3155, Seq. ID No. 3157, Seq. ID No. 3159, Seq. ID No. 3161, Seq. ID No. 3165, Seq. ID No. 3171, Seq. ID No. 3177, Seq. ID No. 3179, Seq. ID No. 3183, Seq. ID No. 3185, Seq. ID No. 3189, Seq. ID No. 3191, Seq. ID No. 3197, Seq. ID No. 3203, Seq. ID No. 3205, Seq. ID No. 3211, Seq. ID No. 3217, Seq. ID No. 3221, Seq. ID No. 3227, Seq. ID No. 3229, Seq. ID No. 3233, Seq. ID No. 3235, Seq. ID No. 3239, Seq. ID No. 3249, Seq. ID No. 3251, Seq. ID No. 3253, Seq. ID No. 3257, Seq. ID No. 3259, Seq. ID No. 3263, Seq. ID No. 3265, Seq. ID No. 3271, Seq. ID No. 3273, Seq. ID No. 3277, Seq. ID No. 3283, Seq. ID No. 3285, Seq. ID No. 3287, Seq. ID No. 3289, Seq. ID No. 3293, Seq. ID No. 3295, Seq. ID No. 3299, Seq. ID No. 3303, Seq. ID No. 3307, Seq. ID No. 3313, Seq. ID No. 3315, Seq. ID No. 3319, Seq. ID No. 3323, Seq. ID No. 3329, Seq. ID No. 3335, Seq. ID No. 3337, Seq. ID No. 3347, Seq. ID No. 3355, Seq. ID No. 3359, Seq. ID No. 3363, Seq. ID No. 3367, Seq. ID No. 3377, Seq. ID No. 3379, Seq. ID No. 3383, Seq. ID No. 3387, Seq. ID No. 3399, Seq. ID No. 3401, Seq. ID No. 3403, Seq. ID No. 3409, Seq. ID No. 3413, Seq. ID No. 3415, Seq. ID No. 3417, Seq. ID No. 3425, Seq. ID No. 3427, Seq. ID No. 3431, Seq. ID No. 3435, Seq. ID No. 3437, Seq. ID No. 3439, Seq. ID No. 3447, Seq. ID No. 3459, Seq. ID No. 3461, Seq. ID No. 3467, Seq. ID No. 3469, Seq. ID No. 3471, Seq. ID No. 3473, Seq. ID No. 3475, Seq. ID No. 3479, Seq. ID No. 3483, Seq. ID No. 3485, Seq. ID No. 3487, Seq. ID No. 3495, Seq. ID No. 3501, Seq. ID No. 3505, Seq. ID No. 3507, Seq. ID No. 3509, Seq. ID No. 3511, Seq. ID No. 3519, Seq. ID No. 3521, Seq. ID No. 3525, Seq. ID No. 3529, Seq. ID No. 3533, Seq. ID No. 3537, Seq. ID No. 3543, Seq. ID No. 3549, Seq. ID No. 3553, Seq. ID No. 3561, Seq. ID No. 3565, Seq. ID No. 3569, Seq. ID No. 3575, Seq. ID No. 3579, Seq. ID No. 3583, Seq. ID No. 3585, Seq. ID No. 3589, Seq. ID No. 3599, Seq. ID No. 3605, Seq. ID No. 3607, Seq. ID No. 3609, Seq. ID No. 3611, Seq. ID No. 3615, Seq. ID No. 3619, Seq. ID No. 3629, Seq. ID No. 3635, Seq. ID No. 3639, Seq. ID No. 3641, Seq. ID No. 3643, Seq. ID No. 3645, Seq. ID No. 3651, Seq. ID No. 3653, Seq. ID No. 3659, Seq. ID No. 3663, Seq. ID No. 3665, Seq. ID No. 3667, Seq. ID No. 3669, Seq. ID No. 3675, Seq. ID No. 3681, Seq. ID No. 3689, Seq. ID No. 3695, Seq. ID No. 3699, Seq. ID No. 3709, Seq. ID No. 3715, Seq. ID No. 3721, Seq. ID No. 3727, Seq. ID No. 3729, Seq. ID No. 3733, Seq. ID No. 3735, Seq. ID No. 3739, Seq. ID No. 3741, Seq. ID No. 3747, Seq. ID No. 3751, Seq. ID No. 3759, Seq. ID No. 3771, Seq. ID No. 3775, Seq. ID No. 3779, Seq. ID No. 3783, Seq. ID No. 3791, Seq. ID No. 3797, Seq. ID No. 3803, Seq. ID No. 3805, Seq. ID No. 3811, Seq. ID No. 3813, Seq. ID No. 3819, Seq. ID No. 3823, Seq. ID No. 3825, Seq. ID No. 3833, Seq. ID No. 3835, Seq. ID No. 3843, Seq. ID No. 3847, Seq. ID No. 3855, Seq. ID No.3857, Seq. ID No. 3863, Seq. ID No. 3867, Seq. ID No. 3873, Seq. ID No. 3877, Seq. ID No. 3881, Seq. ID No. 3883, Seq. ID No. 3885, Seq. ID No. 3893, Seq. ID No. 3897, Seq. ID No. 3901, Seq. ID No. 3907, Seq. ID No. 3913, Seq. ID No. 3915, Seq. ID No. 3917, Seq. ID No. 3919, Seq. ID No. 3925, Seq. ID No. 3929, Seq. ID No. 3933, Seq. ID No. 3935, Seq. ID No. 3937, Seq. ID No. 3941, Seq. ID No. 3943, Seq. ID No. 3945, Seq. ID No. 3949, Seq. ID No. 3951, Seq. ID No. 3955, Seq. ID No. 3957, Seq. ID No. 3971, Seq. ID No. 3975, Seq. ID No. 3983, Seq. ID No. 3985, Seq. ID No. 3987, Seq. ID No. 3991, Seq. ID No. 4003, Seq. ID No. 4009, Seq. ID No. 4013, Seq. ID No. 4015, Seq. ID No. 4017, Seq. ID No. 4021, Seq. ID No. 4023, Seq. ID No. 4029, Seq. ID No. 4031, Seq. ID No. 4035, Seq. ID No. 4037, Seq. ID No. 4039, Seq. ID No. 4049, Seq. ID No. 4055, Seq. ID No. 4057, Seq. ID No. 4061, Seq. ID No. 4063, Seq. ID No. 4067, Seq. ID No. 4069, Seq. ID No. 4075, Seq. ID No. 4077, Seq. ID No. 4079, Seq. ID No. 4087, Seq. ID No. 4089, Seq. ID No. 4095, Seq. ID No. 4097, Seq. ID No. 4099, Seq. ID No. 4105, Seq. ID No. 4111, Seq. ID No. 4117, Seq. ID No. 4121, Seq. ID No. 4123, Seq. ID No. 4127, Seq. ID No. 4129, Seq. ID No. 4133, Seq. ID No. 4137, Seq. ID No. 4145, Seq. ID No. 4149, Seq. ID No. 4157, Seq. ID No. 4161, Seq. ID No. 4163, Seq. ID No. 4167, Seq. ID No. 4173, Seq. ID No. 4179, Seq. ID No. 4185, Seq. ID No. 4191, Seq. ID No. 4193, Seq. ID No. 4195, Seq. ID No. 4197, Seq. ID No. 4199, Seq. ID No. 4201, Seq. ID No. 4203, Seq. ID No. 4205, Seq. ID No. 4211, Seq. ID No. 4215, Seq. ID No. 4217, Seq. ID No. 4219, Seq. ID No. 4235, Seq. ID No. 4237, Seq. ID No. 4239, Seq. ID No. 4243, Seq. ID No. 4251, Seq. ID No. 4255, Seq. ID No. 4263, Seq. ID No. 4267, Seq. ID No. 4271, Seq. ID No. 4273, Seq. ID No. 4279, Seq. ID No. 4281, Seq. ID No. 4283, Seq. ID No. 4291, Seq. ID No. 4295, Seq. ID No. 4299, Seq. ID No. 4301, Seq. ID No. 4303, Seq. ID No. 4307, Seq. ID No. 4309, Seq. ID No. 4313, Seq. ID No. 4317, Seq. ID No. 4323, Seq. ID No. 4325, Seq. ID No. 4329, Seq. ID No. 4331, Seq. ID No. 4333, Seq. ID No. 4339, Seq. ID No. 4343, Seq. ID No. 4347, Seq. ID No. 4353, Seq. ID No. 4355, Seq. ID No. 4357, Seq. ID No. 4365, Seq. ID No. 4369, Seq. ID No. 4373, Seq. ID No. 4375, Seq. ID No. 4377, Seq. ID No. 4379, Seq. ID No. 4381, Seq. ID No. 4383, Seq. ID No. 4391, Seq. ID No. 4393, Seq. ID No. 4397, Seq. ID No. 4401, Seq. ID No. 4411, Seq. ID No. 4417, Seq. ID No. 4423, Seq. ID No. 4431, Seq. ID No. 4433, Seq. ID No. 4435, Seq. ID No. 4443, Seq. ID No. 4447, Seq. ID No. 4451, Seq. ID No. 4459, Seq. ID No. 4461, Seq. ID No. 4467, Seq. ID No. 4471, Seq. ID No. 4475, Seq. ID No. 4479, Seq. ID No. 4481, Seq. ID No. 4487, Seq. ID No. 4491, Seq. ID No. 4501, Seq. ID No. 4505, Seq. ID No. 4511, Seq. ID No. 4523, Seq. ID No. 4531, Seq. ID No. 4533, Seq. ID No. 4537, Seq. ID No. 4539, Seq. ID No. 4541, Seq. ID No. 4543, Seq. ID No. 4547, Seq. ID No. 4549, Seq. ID No. 4559, Seq. ID No. 4565, Seq. ID No. 4567, Seq. ID No. 4571, Seq. ID No. 4575, Seq. ID No. 4577, Seq. ID No. 4581, Seq. ID No. 4585, Seq. ID No. 4591, Seq. ID No. 4595, Seq. ID No. 4597, Seq. ID No. 4603, Seq. ID No. 4607, Seq. ID No. 4609, Seq. ID No. 4611, Seq. ID No. 4615, Seq. ID No. 4623, Seq. ID No. 4627, Seq. ID No. 4631, Seq. ID No. 4635, Seq. ID No. 4637, Seq. ID No. 4643, Seq. ID No. 4649, Seq. ID No. 4651, Seq. ID No. 4653, Seq. ID No. 4657, Seq. ID No. 4661, Seq. ID No. 4663, Seq. ID No. 4669, Seq. ID No. 4671, Seq. ID No. 4673, Seq. ID No. 4679, Seq. ID No. 4685, Seq. ID No. 4693, Seq. ID No. 4697, Seq. ID No. 4703, Seq. ID No. 4705, Seq. ID No. 4713, Seq. ID No. 4717, Seq. ID No. 4719, Seq. ID No. 4725, Seq. ID No. 4729, Seq. ID No. 4731, Seq. ID No. 4733, Seq. ID No. 4739, Seq. ID No. 4743, Seq. ID No. 4745, Seq. ID No. 4747, Seq. ID No. 4749, Seq. ID No. 4751, Seq. ID No. 4757, Seq. ID No. 4765, Seq. ID No. 4769, Seq. ID No. 4777, Seq. ID No. 4779, Seq. ID No. 4781, Seq. ID No. 4787, Seq. ID No. 4789, Seq. ID No. 4791, Seq. ID No. 4797, Seq. ID No. 4803, Seq. ID No. 4807, Seq. ID No. 4809, Seq. ID No. 4811, Seq. ID No. 4813, Seq. ID No. 4815, Seq. ID No. 4821, Seq. ID No. 4823, Seq. ID No. 4825, Seq. ID No. 4827, Seq. ID No. 4833, Seq. ID No. 4835, Seq. ID No. 4837, Seq. ID No. 4843, Seq. ID No. 4845, Seq. ID No. 4847, Seq. ID No. 4849, Seq. ID No. 4859, Seq. ID No. 4861, Seq. ID No. 4865, Seq. ID No. 4867, Seq. ID No. 4875, Seq. ID No. 4877, Seq. ID No. 4879, Seq. ID No. 4881, Seq. ID No. 4883, Seq. ID No. 4885, Seq. ID No. 4887, Seq. ID No. 4893, Seq. ID No. 4899, Seq. ID No. 4901, Seq. ID No. 4903, Seq. ID No. 4905, Seq. ID No. 4907, Seq. ID No. 4911, Seq. ID No. 4915, Seq. ID No. 4917, Seq. ID No. 4919, Seq. ID No. 4921, Seq. ID No. 4927, Seq. ID No. 4931, Seq. ID No. 4935, Seq. ID No. 4939, Seq. ID No. 4941, Seq. ID No. 4943, Seq. ID No. 4945, Seq. ID No. 4947, Seq. ID No. 4953, Seq. ID No. 4957, Seq. ID No. 4959, Seq. ID No. 4963, Seq. ID No. 4965, Seq. ID No. 4969, Seq. ID No. 4971, Seq. ID No. 4973, Seq. ID No. 4979, Seq. ID No. 4985, Seq. ID No. 4989, Seq. ID No. 4991, Seq. ID No. 4995, Seq. ID No. 4997, Seq. ID No. 5001, Seq. ID No. 5007, Seq. ID No. 5009, Seq. ID No. 5017, Seq. ID No. 5021, Seq. ID No. 5027, Seq. ID No. 5029, Seq. ID No. 5039, Seq. ID No. 5047, Seq. ID No. 5051, Seq. ID No. 5053, Seq. ID No. 5055, Seq. ID No. 5059, Seq. ID No. 5061, Seq. ID No. 5069, Seq. ID No. 5077, Seq. ID No. 5079, Seq. ID No. 5081, Seq. ID No. 5083, Seq. ID No. 5087, Seq. ID No. 5091, Seq. ID No. 5093, Seq. ID No. 5097, Seq. ID No. 5103, Seq. ID No. 5105, Seq. ID No. 5109, Seq. ID No. 5111, Seq. ID No. 5113, Seq. ID No. 5115, Seq. ID No. 5117, Seq. ID No. 5127, Seq. ID No. 5141, Seq. ID No. 5145, Seq. ID No. 5149, Seq. ID No. 5153, Seq. ID No. 5157, Seq. ID No. 5165, Seq. ID No. 5169, Seq. ID No. 5171, Seq. ID No. 5177, Seq. ID No. 5183, Seq. ID No. 5185, Seq. ID No. 5187, Seq. ID No. 5193, Seq. ID No. 5195, Seq. ID No. 5201, Seq. ID No. 5203, Seq. ID No. 5205, Seq. ID No. 5209, Seq. ID No. 5211, Seq. ID No. 5215, Seq. ID No. 5219, Seq. ID No. 5227, Seq. ID No. 5231, Seq. ID No. 5235, Seq. ID No. 5237, Seq. ID No. 5239, Seq. ID No. 5243, Seq. ID No. 5245, Seq. ID No. 5247, Seq. ID No. 5253, Seq. ID No. 5255, Seq. ID No. 5257, Seq. ID No. 5261, Seq. ID No. 5263, Seq. ID No. 5271, Seq. ID No. 5275, Seq. ID No. 5283, Seq. ID No. 5291, Seq. ID No. 5299, Seq. ID No. 5305, Seq. ID No. 5309, Seq. ID No. 5311, Seq. ID No. 5313, Seq. ID No. 5319, Seq. ID No. 5323, Seq. ID No. 5325, Seq. ID No. 5335, Seq. ID No. 5337, Seq. ID No. 5341, Seq. ID No. 5345, Seq. ID No. 5351, Seq. ID No. 5355, Seq. ID No. 5363, Seq. ID No. 5365, Seq. ID No. 5367, Seq. ID No. 5371, Seq. ID No. 5373, Seq. ID No. 5381, Seq. ID No. 5383, Seq. ID No. 5387, Seq. ID No. 5389, Seq. ID No. 5391, Seq. ID No. 5393, Seq. ID No. 5395, Seq. ID No. 5397, Seq. ID No. 5399, Seq. ID No. 5401, Seq. ID No. 5403, Seq. ID No. 5409, Seq. ID No. 5411, Seq. ID No. 5415, Seq. ID No. 5417, Seq. ID No. 5419, Seq. ID No. 5421, Seq. ID No. 5423, Seq. ID No. 5427, Seq. ID No. 5431, Seq. ID No. 5433, Seq. ID No. 5439, Seq. ID No. 5441, Seq. ID No. 5443, Seq. ID No. 5447, Seq. ID No. 5449, Seq. ID No. 5451, Seq. ID No. 5453, Seq. ID No. 5455, Seq. ID No. 5457, Seq. ID No. 5473, Seq. ID No. 5477, Seq. ID No. 5483, Seq. ID No. 5487, Seq. ID No. 5491, Seq. ID No. 5495, Seq. ID No. 5499, Seq. ID No. 5503, Seq. ID No. 5507, Seq. ID No. 5509, Seq. ID No. 5513, Seq. ID No. 5527, Seq. ID No. 5531, Seq. ID No. 5537, Seq. ID No. 5539, Seq. ID No. 5541, Seq. ID No. 5547, Seq. ID No. 5553, Seq. ID No. 5557, Seq. ID No. 5559, Seq. ID No. 5563, Seq. ID No. 5567, Seq. ID No. 5571, Seq. ID No. 5575, Seq. ID No. 5581, Seq. ID No. 5585, Seq. ID No. 5591, Seq. ID No. 5599, Seq. ID No. 5601, Seq. ID No. 5605, Seq. ID No. 5615, Seq. ID No. 5621, Seq. ID No. 5629, Seq. ID No. 5631, Seq. ID No. 5637, Seq. ID No. 5647, Seq. ID No. 5651, Seq. ID No. 5655, Seq. ID No. 5657, Seq. ID No. 5661, Seq. ID No. 5665, Seq. ID No. 5669, Seq. ID No. 5671, Seq. ID No. 5673, Seq. ID No. 5679, Seq. ID No. 5685, Seq. ID No. 5693, Seq. ID No. 5697, Seq. ID No. 5699, Seq. ID No. 5701, Seq. ID No. 5707, Seq. ID No. 5715, Seq. ID No. 5717, Seq. ID No. 5723, Seq. ID No. 5727, Seq. ID No. 5735, Seq. ID No. 5739, Seq. ID No. 5749, Seq. ID No. 5751, Seq. ID No. 5755, Seq. ID No. 5761, Seq. ID No. 5771, Seq. ID No. 5775, Seq. ID No. 5777, Seq. ID No. 5779, Seq. ID No. 5783, Seq. ID No. 5789, Seq. ID No. 5795, Seq. ID No. 5801, Seq. ID No. 5803, Seq. ID No. 5809, Seq. ID No. 5815, Seq. ID No. 5819, Seq. ID No. 5825, Seq. ID No. 5833, Seq. ID No. 5835, Seq. ID No. 5841, Seq. ID No. 5845, Seq. ID No. 5847, Seq. ID No. 5849, Seq. ID No. 5851, Seq. ID No. 5853, Seq. ID No. 5855, Seq. ID No. 5859, Seq. ID No. 5865, Seq. ID No. 5871, Seq. ID No. 5875, Seq. ID No. 5877, Seq. ID No. 5881, Seq. ID No. 5887, Seq. ID No. 5891, Seq. ID No. 5895, Seq. ID No. 5897, Seq. ID No. 5901, Seq. ID No. 5903, Seq. ID No. 5911, Seq. ID No. 5913, Seq. ID No. 5915, Seq. ID No. 5917, Seq. ID No. 5919, Seq. ID No. 5929, Seq. ID No. 5931, Seq. ID No. 5933, Seq. ID No. 5937, Seq. ID No. 5941, Seq. ID No. 5943, Seq. ID No. 5947, Seq. ID No. 5951, Seq. ID No. 5953, Seq. ID No. 5955, Seq. ID No. 5957, Seq. ID No. 5961, Seq. ID No. 5969, Seq. ID No. 5973, Seq. ID No. 5979, Seq. ID No. 5981, Seq. ID No. 5985, Seq. ID No. 5989, Seq. ID No. 5993, Seq. ID No. 6001, Seq. ID No. 6003, Seq. ID No. 6011, Seq. ID No. 6015, Seq. ID No. 6017, Seq. ID No. 6021, Seq. ID No. 6027, Seq. ID No. 6029, Seq. ID No. 6033, Seq. ID No. 6035, Seq. ID No. 6041, Seq. ID No. 6043, Seq. ID No. 6053, Seq. ID No. 6057, Seq. ID No. 6065, Seq. ID No. 6071, Seq. ID No. 6079, Seq. ID No. 6085, Seq. ID No. 6089, Seq. ID No. 6095, Seq. ID No. 6097, Seq. ID No. 6101, Seq. ID No. 6103, Seq. ID No. 6111, Seq. ID No. 6113, Seq. ID No. 6117, Seq. ID No,. 6123, Seq. ID No. 6125, Seq. ID No. 6129, Seq. ID No. 6131, Seq. ID No. 6133, Seq. ID No. 6141, Seq. ID No. 6145, Seq. ID No. 6149, Seq. ID No. 6157, Seq. ID No. 6165, Seq. ID No. 6169, Seq. ID No. 6171, Seq. ID No. 6175, Seq. ID No. 6177, Seq. ID No. 6181, Seq. ID No. 6187, Seq. ID No. 6195, Seq. ID No. 6201, Seq. ID No. 6209, Seq. ID No. 6213, Seq. ID No. 6215, Seq. ID No. 6219, Seq. ID No. 6233, Seq. ID No. 6235, Seq. ID No. 6243, Seq. ID No. 6249, Seq. ID No. 6251, Seq. ID No. 6257, Seq. ID No. 6261, Seq. ID No. 6263, Seq. ID No. 6271, Seq. ID No. 6275, Seq. ID No. 6277, Seq. ID No. 6281, Seq. ID No. 6287, Seq. ID No. 6297, Seq. ID No. 6299, Seq. ID No. 6305, Seq. ID No. 6311, Seq. ID No. 6315, Seq. ID No. 6321, Seq. ID No. 6323, Seq. ID No. 6327, Seq. ID No. 6331, Seq. ID No. 6333, Seq. ID No. 6337, Seq. ID No. 6339, Seq. ID No. 6341, Seq. ID No. 6343, Seq. ID No. 6345, Seq. ID No. 6353, Seq. ID No. 6357, Seq. ID No. 6359, Seq. ID No. 6361, Seq. ID No. 6367, Seq. ID No. 6369, Seq. ID No. 6373, Seq. ID No. 6375, Seq. ID No. 6377, Seq. ID No. 6379, Seq. ID No. 6389, Seq. ID No. 6395, Seq. ID No. 6399, Seq. ID No. 6403, Seq. ID No. 6407, Seq. ID No. 6411, Seq. ID No. 6413, Seq. ID No. 6417, Seq. ID No. 6423, Seq. ID No. 6433, Seq. ID No. 6437, Seq. ID. No. 6439, Seq. ID No. 6445, Seq. ID No. 6449, Seq. ID No. 6451, Seq. ID No. 6457, Seq. ID No. 6459, Seq. ID No. 6461, Seq. ID No. 6463, Seq. ID No. 6477, Seq. ID No. 6481, Seq. ID No. 6483, Seq. ID No. 6487, Seq. ID No. 6489, Seq. ID No. 6491, Seq. ID No. 6493, Seq. ID No. 6495, Seq. ID No. 6503, Seq. ID No. 6505, Seq. ID No. 6511, Seq. ID No. 6515, Seq. ID No. 6519, Seq. ID No. 6523, Seq. ID No. 6525, Seq. ID No. 6529, Seq. ID No. 6531, Seq. ID No. 6533, Seq. ID No. 6537, Seq. ID No. 6545, Seq. ID No. 6551, Seq. ID No. 6553, Seq. ID No. 6559, Seq. ID No. 6563, Seq. ID No. 6567, Seq. ID No. 6577, Seq. ID No. 6579, Seq. ID No. 6583, Seq. ID No. 6585, Seq. ID No. 6587, Seq. ID No. 6589, Seq. ID No. 6603, Seq. ID No. 6605, Seq. ID No. 6609, Seq. ID No. 6613, Seq. ID No. 6615, Seq. ID No. 6617, Seq. ID No. 6621, Seq. ID No. 6623, Seq. ID No. 6627, Seq. ID No. 6629, Seq. ID No. 6633, and Seq. ID No. 6639.

In yet another preferred embodiment, the polynucleotide is selected from the Alloiococcus otitidis polynucleotides, which encode polypeptides, identified by GeneMark™ ORF finder program, and the polynucleotide is selected from one of Seq. ID No. 1, Seq. ID No. 3, Seq. ID No. 11, Seq. ID No. 13, Seq. ID No. 15, Seq. ID No. 17, Seq. ID No. 25, Seq. ID No. 27, Seq. ID No. 29, Seq. ID No. 35, Seq. ID No. 37, Seq. ID No. 39, Seq. ID No. 41, Seq. ID No. 43, Seq. ID No. 45, Seq. ID No. 49, Seq. ID No. 53, Seq. ID No. 57, Seq. ID No. 61, Seq. ID No. 69, Seq. ID No. 71, Seq. ID No. 75, Seq. ID No. 81, Seq. ID No. 83, Seq. ID No. 89, Seq. ID No. 95, Seq. ID No. 101, Seq. ID No. 111, Seq. ID No. 119, Seq. ID No. 125, Seq. ID No. 129, Seq. ID No. 135, Seq. ID No. 137, Seq. ID No. 141, Seq. ID No. 147, Seq. ID No. 151, Seq. ID No. 155, Seq. ID No. 167, Seq. ID No. 171, Seq. ID No. 181, Seq. ID No. 185, Seq. ID No. 187, Seq. ID No. 189, Seq. ID No. 191, Seq. ID No. 195, Seq. ID No. 199, Seq. ID No. 207, Seq. ID No. 213, Seq. ID No. 219, Seq. ID No. 221, Seq. ID No. 225, Seq. ID No. 227, Seq. ID No. 229, Seq. ID No. 233, Seq. ID No. 239, Seq. ID No. 241, Seq. ID No. 243, Seq. ID No. 253, Seq. ID No. 261, Seq. ID No. 263, Seq. ID No. 267, Seq. ID No. 273, Seq. ID No. 275, Seq. ID No. 277, Seq. ID No. 279, Seq. ID No. 291, Seq. ID No. 293, Seq. ID No. 295, Seq. ID No. 299, Seq. ID No. 303, Seq. ID No. 309, Seq. ID No. 311, Seq. ID No. 315, Seq. ID No. 319, Seq. ID No. 323, Seq. ID No. 331, Seq. ID No. 341, Seq. ID No. 345, Seq. ID No. 351, Seq. ID No. 355, Seq. ID No. 361, Seq. ID No. 363, Seq. ID No. 365, Seq. ID No. 375, Seq. ID No. 379, Seq. ID No. 381, Seq. ID No. 383, Seq. ID No. 389, Seq. ID No. 391, Seq. ID No. 395, Seq. ID No. 399, Seq. ID No. 405, Seq. ID No. 409, Seq. ID No. 413, Seq. ID No. 417, Seq. ID No. 421, Seq. ID No. 423, Seq. ID No. 425, Seq. ID No. 427, Seq. ID No. 429, Seq. ID No. 435, Seq. ID No. 437, Seq. ID No. 439, Seq. ID No. 441, Seq. ID No. 443, Seq. ID No. 445, Seq. ID No. 447, Seq. ID No. 449, Seq. ID No. 459, Seq. ID No. 465, Seq. ID No. 485, Seq. ID No. 501, Seq. ID No. 505, Seq. ID No. 511, Seq. ID No. 513, Seq. ID No. 517, Seq. ID No. 519, Seq. ID No. 523, Seq. ID No. 529, Seq. ID No. 533, Seq. ID No. 535, Seq. ID No. 541, Seq. ID No. 543, Seq. ID No. 551, Seq. ID No. 553, Seq. ID No. 559, Seq. ID No. 563, Seq. ID No. 569, Seq. ID No. 575, Seq. ID No. 579, Seq. ID No. 583, Seq. ID No. 591, Seq. ID No. 595, Seq. ID No. 601, Seq. ID No. 605, Seq. ID No. 607, Seq. ID No. 611, Seq. ID No. 613, Seq. ID No. 615, Seq. ID No. 621, Seq. ID No. 627, Seq. ID No. 629, Seq. ID No. 633, Seq. ID No. 635, Seq. ID No. 637, Seq. ID No. 643, Seq. ID No. 649, Seq. ID No. 651, Seq. ID No. 655, Seq. ID No. 657, Seq. ID No. 669, Seq. ID No. 671, Seq. ID No. 675, Seq. ID No. 681, Seq. ID No. 683, Seq. ID No. 685, Seq. ID No. 693, Seq. ID No. 703, Seq. ID No. 705, Seq. ID No. 711, Seq. ID No. 725, Seq. ID No. 731, Seq. ID No. 735, Seq. ID No. 737, Seq. ID No. 741, Seq. ID No. 743, Seq. ID No. 749, Seq. ID No. 753, Seq. ID No. 759, Seq. ID No. 773, Seq. ID No. 781, Seq. ID No. 783, Seq. ID No. 785, Seq. ID No. 789, Seq. ID No. 793, Seq. ID No. 797, Seq. ID No. 803, Seq. ID No. 815, Seq. ID No. 819, Seq. ID No. 821, Seq. ID No. 827, Seq. ID No. 831, Seq. ID No. 833, Seq. ID No. 835, Seq. ID No. 837, Seq. ID No. 841, Seq. ID No. 843, Seq. ID No. 849, Seq. ID No. 851, Seq. ID No. 853, Seq. ID No. 857, Seq. ID No. 865, Seq. ID No. 869, Seq. ID No. 873, Seq. ID No. 877, Seq. ID No. 883, Seq. ID No. 887, Seq. ID No. 891, Seq. ID No. 895, Seq. ID No. 901, Seq. ID No. 907, Seq. ID No. 911, Seq. ID No. 917, Seq. ID No. 921, Seq. ID No. 927, Seq. ID No. 935, Seq. ID No. 939, Seq. ID No. 945, Seq. ID No. 951, Seq. ID No. 953, Seq. ID No. 959, Seq. ID No. 963, Seq. ID No. 967, Seq. ID No. 969, Seq. ID No. 977, Seq. ID No. 989, Seq. ID No. 995, Seq. ID No. 999, Seq. ID No. 1005, Seq. ID No. 1007, Seq. ID No. 1015, Seq. ID No. 1021, Seq. ID No. 1035, Seq. ID No. 1041, Seq. ID No. 1043, Seq. ID No. 1049, Seq. ID No. 1053, Seq. ID No. 1057, Seq. ID No. 1059, Seq. ID No. 1063, Seq. ID No. 1067, Seq. ID No. 1069, Seq. ID No. 1071, Seq. ID No. 1075, Seq. ID No. 1083, Seq. ID No. 1085, Seq. ID No. 1089, Seq. ID No. 1093, Seq. ID No. 1097, Seq. ID No. 1099, Seq. ID No. 1101, Seq. ID No. 1109, Seq. ID No. 1113, Seq. ID No. 1117, Seq. ID No. 1121, Seq. ID No. 1129, Seq. ID No. 1133, Seq. ID No. 1137, Seq. ID No. 1141, Seq. ID No. 1145, Seq. ID No. 1149, Seq. ID No. 1155, Seq. ID No. 1157, Seq. ID No. 1159, Seq. ID No. 1163, Seq. ID No. 1171, Seq. ID No. 1173, Seq. ID No. 1175, Seq. ID No. 1181, Seq. ID No. 1185, Seq. ID No. 1189, Seq. ID No. 1199, Seq. ID No. 1205, Seq. ID No. 1209, Seq. ID No. 1215, Seq. ID No. 1219, Seq. ID No. 1223, Seq. ID No. 1225, Seq. ID No. 1233, Seq. ID No. 1235, Seq. ID No. 1241, Seq. ID No. 1251, Seq. ID No. 1255, Seq. ID No. 1261, Seq. ID No. 1271, Seq. ID No. 1281, Seq. ID No. 1315, Seq. ID No. 1323, Seq. ID No. 1331, Seq. ID No. 1333, Seq. ID No. 1339, Seq. ID No. 1345, Seq. ID No. 1347, Seq. ID No. 1355, Seq. ID No. 1357, Seq. ID No. 1369, Seq. ID No. 1373, Seq. ID No. 1387, Seq. ID No. 1391, Seq. ID No. 1395, Seq. ID No. 1397, Seq. ID No. 1403, Seq. ID No. 1405, Seq. ID No. 1407, Seq. ID No. 1413, Seq. ID No. 1415, Seq. ID No. 1417, Seq. ID No. 1425, Seq. ID No. 1427, Seq. ID No. 1429, Seq. ID No. 1431, Seq. ID No. 1433, Seq. ID No. 1435, Seq. ID No. 1439, Seq. ID No. 1441, Seq. ID No. 1451, Seq. ID No. 1453, Seq. ID No. 1459, Seq. ID No. 1461, Seq. ID No. 1469, Seq. ID No. 1471, Seq. ID No. 1473, Seq. ID No. 1475, Seq. ID No. 1477, Seq. ID No. 1483, Seq. ID No. 1487, Seq. ID No. 1489, Seq. ID No. 1493, Seq. ID No. 1495, Seq. ID No. 1497, Seq. ID No. 1501, Seq. ID No. 1503, Seq. ID No. 1507, Seq. ID No. 1509, Seq. ID No. 1517; Seq. ID No. 1521, Seq. ID No. 1527, Seq. ID No. 1531, Seq. ID No. 1537, Seq. ID No. 1541, Seq. ID No. 1543, Seq. ID No. 1549, Seq. ID No. 1561, Seq. ID No. 1563, Seq. ID No. 1565, Seq. ID No. 1569, Seq. ID No. 1573, Seq. ID No. 1575, Seq. ID No. 1581, Seq. ID No. 1587, Seq. ID No. 1591, Seq. ID No. 1595, Seq. ID No. 1597, Seq. ID No. 1605, Seq. ID No. 1609, Seq. ID No. 1613, Seq. ID No. 1619, Seq. ID No. 1623, Seq. ID No. 1627, Seq. ID No. 1629, Seq. ID No. 1635, Seq. ID No. 1641, Seq. ID No. 1647, Seq. ID No. 1651, Seq. ID No. 1657, Seq. ID No. 1659, Seq. ID No. 1667, Seq. ID No. 1669, Seq. ID No. 1679, Seq. ID No. 1683, Seq. ID No. 1687, Seq. ID No. 1691, Seq. ID No. 1697, Seq. ID No. 1699, Seq. ID No. 1701, Seq. ID No. 1707, Seq. ID No. 1709, Seq. ID No. 1713, Seq. ID No. 1717, Seq. ID No. 1721, Seq. ID No. 1725, Seq. ID No. 1735, Seq. ID No. 1737, Seq. ID No. 1739, Seq. ID No. 1743, Seq. ID No. 1745, Seq. ID No. 1749, Seq. ID No. 1755, Seq. ID No. 1769, Seq. ID No. 1773, Seq. ID No. 1777, Seq. ID No. 1785, Seq. ID No. 1789, Seq. ID No. 1791, Seq. ID No. 1797, Seq. ID No. 1799, Seq. ID No. 1803, Seq. ID No. 1805, Seq. ID No. 1807, Seq. ID No. 1817, Seq. ID No. 1819, Seq. ID No. 1821, Seq. ID No. 1825, Seq. ID No. 1827, Seq. ID No. 1831, Seq. ID No. 1835, Seq. ID No. 1841, Seq. ID No. 1845, Seq. ID No. 1847, Seq. ID No. 1849, Seq. ID No. 1851, Seq. ID No. 1855, Seq. ID No. 1859, Seq. ID No. 1861, Seq. ID No. 1865, Seq. ID No. 1867, Seq. ID No. 1871, Seq. ID No. 1873, Seq. ID No. 1877, Seq. ID No. 1879, Seq. ID No. 1887, Seq. ID No. 1889, Seq. ID No. 1895, Seq. ID No. 1897, Seq. ID No. 1899, Seq. ID No. 1901, Seq. ID No. 1905, Seq. ID No. 1909, Seq. ID No. 1917, Seq. ID No. 1921, Seq. ID No. 1925, Seq. ID No. 1931, Seq. ID No. 1933, Seq. ID No. 1937, Seq. ID No. 1939, Seq. ID No. 1945, Seq. ID No. 1947, Seq. ID No. 1951, Seq. ID No. 1955, Seq. ID No. 1959, Seq. ID No. 1961, Seq. ID No. 1965, Seq. ID No. 1967, Seq. ID No. 1969, Seq. ID No. 1973, Seq. ID No. 1977, Seq. ID No. 1979, Seq. ID No. 1983, Seq. ID No. 1989, Seq. ID No. 1991, Seq. ID No. 1997, Seq. ID No. 2001, Seq. ID No. 2003, Seq. ID No. 2011, Seq. ID No. 2021, Seq. ID No. 2023, Seq. ID No. 2029, Seq. ID No. 2033, Seq. ID No. 2037, Seq. ID No. 2039, Seq. ID No. 2045, Seq. ID No. 2047, Seq. ID No. 2051, Seq. ID No. 2057, Seq. ID No. 2061, Seq. ID No. 2067, Seq. ID No. 2069, Seq. ID No. 2071, Seq. ID No. 2073, Seq. ID No. 2077, Seq. ID No. 2089, Seq. ID No. 2093, Seq. ID No. 2095, Seq. ID No. 2099, Seq. ID No. 2101, Seq. ID No. 2113, Seq. ID No. 2117, Seq. ID No. 2123, Seq. ID No. 2135, Seq. ID No. 2137, Seq. ID No. 2139, Seq. ID No. 2143, Seq. ID No. 2147, Seq. ID No. 2153, Seq. ID No. 2159, Seq. ID No. 2163, Seq. ID No. 2167, Seq. ID No. 2175, Seq. ID No. 2181, Seq. ID No. 2185, Seq. ID No. 2189, Seq. ID No. 2193, Seq. ID No. 2195, Seq. ID No. 2197, Seq. ID No. 2205, Seq. ID No. 2217, Seq. ID No. 2219, Seq. ID No. 2223, Seq. ID No. 2225, Seq. ID No. 2229, Seq. ID No. 2235, Seq. ID No. 2239, Seq. ID No. 2243, Seq. ID No. 2245, Seq. ID No. 2247, Seq. ID No. 2253, Seq. ID No. 2255, Seq. ID No. 2257, Seq. ID No. 2259, Seq. ID No. 2261, Seq. ID No. 2265, Seq. ID No. 2269, Seq. ID No. 2273, Seq. ID No. 2285, Seq. ID No. 2297, Seq. ID No. 2299, Seq. ID No. 2301, Seq. ID No. 2313, Seq. ID No. 2327, Seq. ID No. 2331, Seq. ID No. 2335, Seq. ID No. 2337, Seq. ID No. 2341, Seq. ID No. 2343, Seq. ID No. 2345, Seq. ID No. 2349, Seq. ID No. 2351, Seq. ID No. 2357, Seq. ID No. 2359, Seq. ID No. 2361, Seq. ID No. 2365, Seq. ID No. 2369, Seq. ID No. 2373, Seq. ID No. 2377, Seq. ID No. 2383, Seq. ID No. 2397, Seq. ID No. 2401, Seq. ID No. 2403, Seq. ID No. 2409, Seq. ID No. 2411, Seq. ID No. 2417, Seq. ID No. 2421, Seq. ID No. 2423, Seq. ID No. 2425, Seq. ID No. 2429, Seq. ID No. 2435, Seq. ID No. 2437, Seq. ID No. 2439, Seq. ID No. 2441, Seq. ID No. 2445, Seq. ID No. 2449, Seq. ID No. 2451, Seq. ID No. 2453, Seq. ID No. 2461, Seq. ID No. 2465, Seq. ID No. 2467, Seq. ID No. 2471, Seq. ID No. 2473, Seq. ID No. 2477, Seq. ID No. 2479, Seq. ID No. 2481, Seq. ID No. 2485, Seq. ID No. 2487, Seq. ID No. 2495, Seq. ID No. 2505, Seq. ID No. 2507, Seq. ID No. 2509, Seq. ID No. 2519, Seq. ID No. 2525, Seq. ID No. 2527, Seq. ID No. 2529, Seq. ID No. 2535, Seq. ID No. 2537, Seq. ID No. 2539, Seq. ID No. 2547, Seq. ID No. 2551, Seq. ID No. 2563, Seq. ID No. 2565, Seq. ID No. 2573, Seq. ID No. 2577, Seq. ID No. 2579, Seq. ID No. 2585, Seq. ID No. 2593, Seq. ID No. 2595, Seq. ID No. 2599, Seq. ID No. 2607, Seq. ID No. 2609, Seq. ID No. 2613, Seq. ID No. 2615, Seq. ID No. 2619, Seq. ID No. 2623, Seq. ID No. 2625, Seq. ID No. 2631, Seq. ID No. 2633, Seq. ID No. 2639, Seq. ID No. 2641, Seq. ID No. 2643, Seq. ID No. 2647, Seq. ID No. 2651, Seq. ID No. 2655, Seq. ID No. 2657, Seq. ID No. 2661, Seq. ID No. 2665, Seq. ID No. 2669, Seq. ID No. 2671, Seq. ID No. 2673, Seq. ID No. 2681, Seq. ID No. 2689, Seq. ID No. 2693, Seq. ID No. 2699, Seq. ID No. 2703, Seq. ID No. 2709, Seq. ID No. 2711, Seq. ID No. 2713, Seq. ID No. 2719, Seq. ID No. 2721, Seq. ID No. 2727, Seq. ID No. 2737, Seq. ID No. 2741, Seq. ID No. 2745, Seq. ID No. 2749, Seq. ID No. 2753, Seq. ID No. 2757, Seq. ID No. 2761, Seq. ID No. 2765, Seq. ID No. 2767, Seq. ID No. 2769, Seq. ID No. 2773, Seq. ID No. 2775, Seq. ID No. 2777, Seq. ID No. 2787, Seq. ID No. 2789, Seq. ID No. 2791, Seq. ID No. 2793, Seq. ID No. 2795, Seq. ID No. 2799, Seq. ID No. 2803, Seq. ID No. 2815, Seq. ID No. 2817, Seq. ID No. 2819, Seq. ID No. 2825, Seq. ID No. 2843, Seq. ID No. 2847, Seq. ID No. 2849, Seq. ID No. 2851, Seq. ID No. 2857, Seq. ID No. 2859, Seq. ID No. 2863, Seq. ID No. 2867, Seq. ID No. 2869, Seq. ID No. 2871, Seq. ID No. 2877, Seq. ID No. 2879, Seq. ID No. 2889, Seq. ID No. 2891, Seq. ID No. 2897, Seq. ID No. 2901, Seq. ID No. 2903, Seq. ID No. 2907, Seq. ID No. 2909, Seq. ID No. 2913, Seq. ID No. 2915, Seq. ID No. 2919, Seq. ID No. 2921, Seq. ID No. 2923, Seq. ID No. 2925, Seq. ID No. 2929, Seq. ID No. 2935, Seq. ID No. 2943, Seq. ID No. 2951, Seq. ID No. 2955, Seq. ID No. 2963, Seq. ID No. 2965, Seq. ID No. 2971, Seq. ID No. 2973; Seq. ID No. 2975, Seq. ID No. 2979, Seq. ID No. 2981, Seq. ID No. 2985, Seq. ID No. 2989, Seq. ID No. 2993, Seq. ID No. 2999, Seq. ID No. 3001, Seq. ID No. 3003, Seq. ID No. 3017, Seq. ID No. 3027, Seq. ID No. 3033, Seq. ID No. 3035, Seq. ID No. 3039, Seq. ID No. 3041, Seq. ID No. 3043, Seq. ID No. 3045, Seq. ID No. 3047, Seq. ID No. 3055, Seq. ID No. 3057, Seq. ID No. 3061, Seq. ID No. 3065, Seq. ID No. 3069, Seq. ID No. 3079, Seq. ID No. 3089, Seq. ID No. 3091, Seq. ID No. 3093, Seq. ID No. 3095, Seq. ID No. 3103, Seq. ID No. 3105, Seq. ID No. 3109, Seq. ID No. 3113, Seq. ID No. 3125, Seq. ID No. 3127, Seq. ID No. 3135, Seq. ID No. 3137, Seq. ID No. 3141, Seq. ID No. 3143, Seq. ID No. 3147, Seq. ID No. 3155, Seq. ID No. 3157, Seq. ID No. 3159, Seq. ID No. 3161, Seq. ID No. 3165, Seq. ID No. 3169, Seq. ID No. 3175, Seq. ID No. 3179, Seq. ID No. 3183, Seq. ID No. 3185, Seq. ID No. 3189, Seq. ID No. 3191, Seq. ID No. 3195, Seq. ID No. 3199, Seq. ID No. 3205, Seq. ID No. 3211, Seq. ID No. 3217, Seq. ID No. 3221, Seq. ID No. 3225, Seq. ID No. 3229, Seq. ID No. 3231, Seq. ID No. 3237, Seq. ID No. 3239, Seq. ID No. 3247, Seq. ID No. 3251, Seq. ID No. 3253, Seq. ID No. 3255, Seq. ID No. 3259, Seq. ID No. 3261, Seq. ID No. 3267, Seq. ID No. 3273, Seq. ID No. 3277, Seq. ID No. 3279, Seq. ID No. 3285, Seq. ID No. 3287, Seq. ID No. 3289, Seq. ID No. 3293, Seq. ID No. 3295, Seq. ID No. 3299, Seq. ID No. 3301, Seq. ID No. 3305, Seq. ID No. 3309, Seq. ID No. 3315, Seq. ID No. 3319, Seq. ID No. 3321, Seq. ID No. 3327, Seq. ID No. 3333, Seq. ID No. 3337, Seq. ID No. 3343, Seq. ID No. 3353, Seq. ID No. 3359, Seq. ID No. 3361, Seq. ID No. 3365, Seq. ID No. 3373, Seq. ID No. 3381, Seq. ID No. 3385, Seq. ID No. 3393, Seq. ID No. 3401, Seq. ID No. 3407, Seq. ID No. 3411, Seq. ID No. 3415, Seq. ID No. 3417, Seq. ID No. 3423, Seq. ID No. 3429, Seq. ID No. 3437, Seq. ID No. 3439, Seq. ID No. 3443, Seq. ID No. 3459, Seq. ID No. 3461, Seq. ID No. 3465, Seq. ID No. 3469, Seq. ID No. 3471, Seq. ID No. 3473, Seq. ID No. 3475, Seq. ID No. 3477, Seq. ID No. 3483, Seq. ID No. 3487, Seq. ID No. 3495, Seq. ID No. 3497, Seq. ID No. 3505, Seq. ID No. 3507, Seq. ID No. 3509, Seq. ID No. 3511, Seq. ID No. 3515, Seq. ID No. 3521, Seq. ID No. 3523, Seq. ID No. 3529, Seq. ID No. 3535, Seq. ID No. 3541, Seq. ID No. 3551, Seq. ID No. 3559, Seq. ID No. 3563, Seq. ID No. 3567, Seq. ID No. 3575, Seq. ID No. 3577, Seq. ID No. 3583, Seq. ID No. 3585, Seq. ID No. 3589, Seq. ID No. 3591, Seq. ID No. 3599, Seq. ID No. 3601, Seq. ID No. 3607, Seq. ID No. 3609, Seq. ID No. 3611, Seq. ID No. 3615, Seq. ID No. 3619, Seq. ID No. 3629, Seq. ID No. 3631, Seq. ID No. 3633, Seq. ID No. 3637, Seq. ID No. 3641, Seq. ID No. 3643, Seq. ID No. 3645, Seq. ID No. 3651, Seq. ID No. 3653, Seq. ID No. 3655, Seq. ID No. 3661, Seq. ID No. 3665, Seq. ID No. 3667, Seq. ID No. 3669, Seq. ID No. 3675, Seq. ID No. 3681, Seq. ID No. 3689, Seq. ID No. 3695, Seq. ID No. 3697, Seq. ID No. 3705, Seq. ID No. 3711, Seq. ID No. 3719, Seq. ID No. 3725, Seq. ID No. 3729, Seq. ID No. 3731, Seq. ID No. 3735, Seq. ID No. 3739, Seq. ID No. 3741, Seq. ID No. 3743, Seq. ID No. 3749, Seq. ID No. 3759, Seq. ID No. 3769, Seq. ID No. 3773, Seq. ID No. 3777, Seq. ID No. 3781, Seq. ID No. 3787, Seq. ID No. 3795, Seq. ID No. 3801, Seq. ID No. 3805, Seq. ID No. 3809, Seq. ID No. 3813, Seq. ID No. 3819, Seq. ID No. 3821, Seq. ID No. 3825, Seq. ID No. 3827, Seq. ID No. 3829, Seq. ID No. 3831, Seq. ID No. 3835, Seq. ID No. 3841, Seq. ID No. 3845, Seq. ID No. 3855, Seq. ID No. 3857, Seq. ID No. 3863, Seq. ID No. 3867, Seq. ID No. 3869, Seq. ID No. 3875, Seq. ID No. 3881, Seq. ID No. 3883, Seq. ID No. 3885, Seq. ID No. 3887, Seq. ID No. 3897, Seq. ID No. 3901, Seq. ID No. 3905, Seq. ID No. 3909, Seq. ID No. 3913, Seq. ID No. 3915, Seq. ID No. 3917, Seq. ID No. 3919, Seq. ID No. 3921, Seq. ID No. 3929, Seq. ID No. 3931, Seq. ID No. 3935, Seq. ID No. 3941, Seq. ID No. 3943, Seq. ID No. 3945, Seq. ID No. 3947, Seq. ID No. 3951, Seq. ID No. 3953, Seq. ID No. 3957, Seq. ID No. 3971, Seq. ID No. 3977, Seq. ID No. 3981, Seq. ID No. 3985, Seq. ID No. 3987, Seq. ID No. 3989, Seq. ID No. 3993, Seq. ID No. 4001, Seq. ID No. 4005, Seq. ID No. 4011, Seq. ID No. 4015, Seq. ID No. 4017, Seq. ID No. 4019, Seq. ID No. 4023, Seq. ID No. 4029, Seq. ID No. 4031, Seq. ID No. 4033, Seq. ID No. 4037, Seq. ID No. 4039, Seq. ID No. 4049, Seq. ID No. 4051, Seq. ID No. 4057, Seq. ID No. 4059, Seq. ID No. 4065, Seq. ID No. 4069, Seq. ID No. 4075, Seq. ID No. 4079, Seq. ID No. 4085, Seq. ID No. 4089, Seq. ID No. 4095, Seq. ID No. 4097, Seq. ID No. 4099, Seq. ID No. 4105, Seq. ID No. 4111, Seq. ID No. 4115, Seq. ID No. 4121, Seq. ID No. 4123, Seq. ID No. 4127, Seq. ID No. 4129, Seq. ID No. 4131, Seq. ID No. 4137, Seq. ID No. 4141, Seq. ID No. 4147, Seq. ID No. 4157, Seq. ID No. 4159, Seq. ID No. 4163, Seq. ID No. 4165, Seq. ID No. 4169, Seq. ID No. 4179, Seq. ID No. 4183, Seq. ID No. 4187, Seq. ID No. 4193, Seq. ID No. 4195, Seq. ID No. 4197, Seq. ID No. 4199, Seq. ID No. 4201, Seq. ID No. 4205, Seq. ID No. 4207, Seq. ID No. 4213, Seq. ID No. 4219, Seq. ID No. 4235, Seq. ID No. 4237, Seq. ID No. 4239, Seq. ID No. 4243, Seq. ID No. 4245, Seq. ID No. 4255, Seq. ID No. 4259, Seq. ID No. 4267, Seq. ID No. 4271, Seq. ID No. 4277, Seq. ID No. 4279, Seq. ID No. 4281, Seq. ID No. 4283, Seq. ID No. 4289, Seq. ID No. 4293, Seq. ID No. 4299, Seq. ID No. 4301, Seq. ID No. 4303, Seq. ID No. 4305, Seq. ID No. 4307, Seq. ID No. 4309, Seq. ID No. 4313, Seq. ID No. 4315, Seq. ID No. 4323, Seq. ID No. 4325, Seq. ID No. 4327, Seq. ID No. 4331, Seq. ID No. 4333, Seq. ID No. 4337, Seq. ID No. 4341, Seq. ID No. 4345, Seq. ID No. 4349, Seq. ID No. 4355, Seq. ID No. 4357, Seq. ID No. 4365, Seq. ID No. 4367, Seq. ID No. 4371, Seq. ID No. 4375, Seq. ID No. 4377, Seq. ID No. 4379, Seq. ID No. 4383, Seq. ID No. 4387, Seq. ID No. 4393, Seq. ID No. 4397, Seq. ID No. 4401, Seq. ID No. 4409, Seq. ID No. 4413, Seq. ID No. 4421, Seq. ID No. 4433, Seq. ID No. 4435, Seq. ID No. 4445, Seq. ID No. 4449, Seq. ID No. 4457, Seq. ID No. 4461, Seq. ID No. 4463, Seq. ID No. 4471, Seq. ID No. 4475, Seq. ID No. 4477, Seq. ID No. 4481, Seq. ID No. 4487, Seq. ID No. 4491, Seq. ID No. 4499, Seq. ID No. 4501, Seq. ID No. 4503, Seq. ID No. 4509, Seq. ID No. 4517, Seq. ID No. 4531, Seq. ID No. 4533, Seq. ID No. 4535, Seq. ID No. 4539, Seq. ID No. 4541, Seq. ID No. 4545, Seq. ID No. 4549, Seq. ID No. 4557, Seq. ID No. 4563, Seq. ID No. 4567, Seq. ID No. 4569, Seq. ID No. 4575, Seq. ID No. 4577, Seq. ID No. 4579, Seq. ID No. 4583, Seq. ID No. 4591, Seq. ID No. 4593, Seq. ID No. 4597, Seq. ID No. 4601, Seq. ID No. 4605, Seq. ID No. 4609, Seq. ID No. 4611, Seq. ID No. 4613, Seq. ID No. 4623, Seq. ID No. 4625, Seq. ID No. 4629, Seq. ID No. 4633, Seq. ID No. 4637, Seq. ID No. 4641, Seq. ID No. 4645, Seq. ID No. 4651, Seq. ID No. 4653, Seq. ID No. 4655, Seq. ID No. 4661, Seq. ID No. 4663, Seq. ID No. 4669, Seq. ID No. 4671, Seq. ID No. 4673, Seq. ID No. 4675, Seq. ID No. 4685, Seq. ID No. 4689, Seq. ID No. 4697, Seq. ID No. 4703, Seq. ID No. 4705, Seq. ID No. 4713, Seq. ID No. 4717, Seq. ID No. 4719, Seq. ID No. 4723, Seq. ID No. 4729, Seq. ID No. 4731, Seq. ID No. 4733, Seq. ID No. 4739, Seq. ID No. 4743, Seq. ID No. 4745, Seq. ID No. 4747, Seq. ID No. 4749, Seq. ID No. 4751, Seq. ID No. 4753, Seq. ID No. 4761, Seq. ID No. 4767, Seq. ID No. 4771, Seq. ID No. 4779, Seq. ID No. 4781, Seq. ID No. 4783, Seq. ID No. 4785, Seq. ID No. 4789, Seq. ID No. 4791, Seq. ID No. 4793, Seq. ID No. 4799, Seq. ID No. 4807, Seq. ID No. 4809, Seq. ID No. 4811, Seq. ID No. 4813, Seq. ID No. 4815, Seq. ID No. 4821, Seq. ID No. 4825, Seq. ID No. 4827, Seq. ID No. 4829, Seq. ID No. 4837, Seq. ID No. 4839, Seq. ID No. 4845, Seq. ID No. 4847, Seq. ID No. 4849, Seq. ID No. 4857, Seq. ID No. 4861, Seq. ID No. 4865, Seq. ID No. 4867, Seq. ID No. 4875, Seq. ID No. 4879, Seq. ID No. 4881, Seq. ID No. 4883, Seq. ID No. 4885, Seq. ID No. 4887, Seq. ID No. 4893, Seq. ID No. 4899, Seq. ID No. 4901, Seq. ID No. 4903, Seq. ID No. 4905, Seq. ID No. 4907, Seq. ID No. 4909, Seq. ID No. 4915, Seq. ID No. 4917, Seq. ID No. 4919, Seq. ID No. 4921, Seq. ID No. 4925, Seq. ID No. 4929, Seq. ID No. 4933, Seq. ID No. 4937, Seq. ID No. 4941, Seq. ID No. 4943, Seq. ID No. 4945, Seq. ID No. 4947, Seq. ID No. 4953, Seq. ID No. 4955, Seq. ID No. 4959, Seq. ID No. 4961, Seq. ID No. 4965, Seq. ID No. 4967, Seq. ID No. 4971, Seq. ID No. 4973, Seq. ID No. 4977, Seq. ID No. 4985, Seq. ID No. 4989, Seq. ID No. 4991, Seq. ID No. 4995, Seq. ID No. 4997, Seq. ID No. 5005, Seq. ID No. 5009, Seq. ID No. 5015, Seq. ID No. 5019, Seq. ID No. 5025, Seq. ID No. 5029, Seq. ID No. 5037, Seq. ID No. 5045, Seq. ID No. 5049, Seq. ID No. 5053, Seq. ID No. 5055, Seq. ID No. 5059, Seq. ID No. 5061, Seq. ID No. 5065, Seq. ID No. 5071, Seq. ID No. 5073, Seq. ID No. 5079, Seq. ID No. 5081, Seq. ID No. 5083, Seq. ID No. 5087, Seq. ID No. 5089, Seq. ID No. 5093, Seq. ID No. 5095, Seq. ID No. 5103, Seq. ID No. 5105, Seq. ID No. 5107, Seq. ID No. 5111, Seq. ID No. 5113, Seq. ID No. 5115, Seq. ID No. 5117, Seq. ID No. 5119, Seq. ID No. 5141, Seq. ID No. 5143, Seq. ID No. 5147, Seq. ID No. 5153, Seq. ID No. 5155, Seq. ID No. 5163, Seq. ID No. 5169, Seq. ID No. 5171, Seq. ID No. 5173, Seq. ID No. 5179, Seq. ID No. 5185, Seq. ID No. 5187, Seq. ID No. 5193, Seq. ID No. 5195, Seq. ID No. 5201, Seq. ID No. 5203, Seq. ID No. 5205, Seq. ID No. 5209, Seq. ID No. 5211, Seq. ID No. 5213, Seq. ID No. 5217, Seq. ID No. 5225, Seq. ID No. 5231, Seq. ID No. 5233, Seq. ID No. 5237, Seq. ID No. 5239, Seq. ID No. 5245, Seq. ID No. 5247, Seq. ID No. 5249, Seq. ID No. 5255, Seq. ID No. 5257, Seq. ID No. 5259, Seq. ID No. 5263, Seq. ID No. 5269, Seq. ID No. 5273, Seq. ID No. 5283, Seq. ID No. 5289, Seq. ID No. 5293, Seq. ID No. 5307, Seq. ID No. 5311, Seq. ID No. 5313, Seq. ID No. 5315, Seq. ID No. 5323, Seq. ID No. 5325, Seq. ID No. 5335, Seq. ID No. 5337, Seq. ID No. 5339, Seq. ID No. 5345, Seq. ID No. 5347, Seq. ID No. 5349, Seq. ID No. 5353, Seq. ID No. 5361, Seq. ID No. 5365, Seq. ID No. 5367, Seq. ID No. 5369, Seq. ID No. 5373, Seq. ID No. 5375, Seq. ID No. 5387, Seq. ID No. 5389, Seq. ID No. 5391, Seq. ID No. 5393, Seq. ID No. 5395, Seq. ID No. 5397, Seq. ID No. 5399, Seq. ID No. 5401, Seq. ID No. 5403, Seq. ID No. 5405, Seq. ID No. 5411, Seq. ID No. 5415, Seq. ID No. 5417, Seq. ID No. 5419, Seq. ID No. 5421, Seq. ID No. 5423, Seq. ID No. 5429, Seq. ID No. 5433, Seq. ID No. 5441, Seq. ID No. 5443, Seq. ID No. 5445, Seq. ID No. 5449, Seq. ID No. 5451, Seq. ID No. 5453, Seq. ID No. 5455, Seq. ID No. 5457, Seq. ID No. 5469, Seq. ID No. 5475, Seq. ID No. 5483, Seq. ID No. 5485, Seq. ID No. 5489, Seq. ID No. 5493, Seq. ID No. 5499, Seq. ID No. 5501, Seq. ID No. 5505, Seq. ID No. 5509, Seq. ID No. 5511, Seq. ID No. 5523, Seq. ID No. 5529, Seq. ID No. 5535, Seq. ID No. 5539, Seq. ID No. 5541, Seq. ID No. 5547, Seq. ID No. 5551, Seq. ID No. 5555, Seq. ID No. 5559, Seq. ID No. 5561, Seq. ID No. 5565, Seq. ID No. 5571, Seq. ID No. 5573, Seq. ID No. 5579, Seq. ID No. 5583, Seq. ID No. 5589, Seq. ID No. 5599, Seq. ID No. 5601, Seq. ID No. 5603, Seq. ID No. 5613, Seq. ID No. 5621, Seq. ID No. 5627, Seq. ID No. 5631, Seq. ID No. 5635, Seq. ID No. 5645, Seq. ID No. 5651, Seq. ID No. 5653, Seq. ID No. 5657, Seq. ID No. 5661, Seq. ID No. 5663, Seq. ID No. 5665, Seq. ID No. 5669, Seq. ID No. 5671, Seq. ID No. 5673, Seq. ID No. 5675, Seq. ID No. 5681, Seq. ID No. 5693, Seq. ID No. 5695, Seq. ID No. 5699, Seq. ID No. 5701, Seq. ID No. 5707, Seq. ID No. 5713, Seq. ID No. 5717, Seq. ID No. 5723, Seq. ID No. 5725, Seq. ID No. 5731, Seq. ID No. 5737, Seq. ID No. 5747, Seq. ID No. 5751, Seq. ID No. 5755, Seq. ID No. 5759, Seq. ID No. 5771, Seq. ID No. 5773, Seq. ID No. 5777, Seq. ID No. 5779, Seq. ID No. 5781, Seq. ID No. 5785, Seq. ID No. 5793, Seq. ID No. 5799, Seq. ID No. 5805, Seq. ID No. 5811, Seq. ID No. 5815, Seq. ID No. 5817, Seq. ID No. 5823, Seq. ID No. 5831, Seq. ID No. 5835, Seq. ID No. 5839, Seq. ID No. 5843, Seq. ID No. 5847, Seq. ID No. 5849, Seq. ID No. 5851, Seq. ID No. 5853, Seq. ID No. 5855, Seq. ID No. 5857, Seq. ID No. 5861, Seq. ID No. 5867, Seq. ID No. 5875, Seq. ID No. 5877, Seq. ID No. 5881, Seq. ID No. 5889, Seq. ID No. 5895, Seq. ID No. 5897, Seq. ID No. 5901, Seq. ID No. 5903, Seq. ID No. 5907, Seq. ID No. 5913, Seq. ID No. 5915, Seq. ID No. 5917, Seq. ID No. 5919, Seq. ID No. 5925, Seq. ID No. 5931, Seq. ID No. 5933, Seq. ID No. 5935, Seq. ID No. 5941, Seq. ID No. 5943, Seq. ID No. 5945, Seq. ID No. 5949, Seq. ID No. 5955, Seq. ID No. 5957, Seq. ID No. 5958, Seq. ID No. 5967, Seq. ID No. 5971, Seq. ID No. 5975, Seq. ID No. 5977, Seq. ID No. 5981, Seq. ID No. 5983, Seq. ID No. 5985, Seq. ID No. 5989, Seq. ID No. 5991, Seq. ID No. 5995, Seq. ID No. 5997, Seq. ID No. 6003, Seq. ID No. 6005, Seq. ID No. 6007, Seq. ID No. 6015, Seq. ID No. 6017, Seq. ID No. 6019, Seq. ID No. 6023, Seq. ID No. 6029, Seq. ID No. 6033, Seq. ID No. 6035, Seq. ID No. 6037, Seq. ID No. 6043, Seq. ID No. 6049, Seq. ID No. 6055, Seq. ID No. 6065, Seq. ID No. 6069, Seq. ID No. 6075, Seq. ID No. 6081, Seq. ID No. 6083, Seq. ID No. 6087, Seq. ID No. 6095, Seq. ID No. 6097, Seq. ID No. 6099, Seq. ID No. 6103, Seq. ID No. 6105, Seq. ID No. 6113, Seq. ID No. 6115, Seq. ID No. 6119, Seq. ID No. 6125, Seq. ID No. 6127, Seq. ID No. 6129, Seq. ID No. 6131, Seq. ID No. 6133, Seq. ID No. 6141, Seq. ID No. 6143, Seq. ID No. 6147, Seq. ID No. 6153, Seq. ID No. 6165, Seq. ID No. 6169, Seq. ID No. 6171, Seq. ID No. 6173, Seq. ID No. 6177, Seq. ID No. 6179, Seq. ID No. 6187, Seq. ID No. 6193, Seq. ID No. 6201, Seq. ID No. 6207, Seq. ID No. 6211, Seq. ID No. 6215, Seq. ID No. 6219, Seq. ID No. 6231, Seq. ID No. 6235, Seq. ID No. 6243, Seq. ID No. 6247, Seq. ID No. 6251, Seq. ID No. 6255, Seq. ID No. 6261, Seq. ID No. 6263, Seq. ID No. 6269, Seq. ID No. 6275, Seq. ID No. 6277, Seq. ID No. 6279, Seq. ID No. 6287, Seq. ID No. 6291, Seq. ID No. 6299, Seq. ID No. 6305, Seq. ID No. 6307, Seq. ID No. 6313, Seq. ID No. 6315, Seq. ID No. 6321, Seq. ID No. 6323, Seq. ID No. 6325, Seq. ID No. 6331, Seq. ID No. 6335, Seq. ID No. 6339, Seq. ID No. 6341, Seq. ID No. 6343, Seq. ID No. 6345, Seq. ID No. 6347, Seq. ID No. 6355, Seq. ID No. 6359, Seq. ID No. 6361, Seq. ID No. 6365, Seq. ID No. 6369, Seq. ID No. 6371, Seq. ID No. 6375, Seq. ID No. 6377, Seq. ID No. 6379, Seq. ID No. 6387, Seq. ID No. 6391, Seq. ID No. 6397, Seq. ID No. 6401, Seq. ID No. 6405, Seq. ID No. 6409, Seq. ID No. 6413, Seq. ID No. 6417, Seq. ID No. 6419, Seq. ID No. 6423, Seq. ID No. 6429, Seq. ID No. 6433, Seq. ID No. 6435, Seq. ID No. 6439, Seq. ID No. 6445, Seq. ID No. 6447, Seq. ID No. 6451, Seq. ID No. 6455, Seq. ID No. 6459, Seq. ID No. 6461, Seq. ID No. 6463, Seq. ID No. 6475, Seq. ID No. 6481, Seq. ID No. 6483, Seq. ID No. 6485, Seq. ID No. 6487, Seq. ID No. 6489, Seq. ID No. 6491, Seq. ID No. 6493, Seq. ID No. 6495, Seq. ID No. 6499, Seq. ID No. 6505, Seq. ID No. 6509, Seq. ID No. 6513, Seq. ID No. 6519, Seq. ID No. 6521, Seq. ID No. 6525, Seq. ID No. 6527, Seq. ID No. 6531, Seq. ID No. 6533, Seq. ID No. 6535, Seq. ID No. 6545, Seq. ID No. 6551, Seq. ID No. 6553, Seq. ID No. 6559, Seq. ID No. 6561, Seq. ID No. 6563, Seq. ID No. 6565, Seq. ID No. 6567, Seq. ID No. 6569, Seq. ID No. 6575, Seq. ID No. 6579, Seq. ID No. 6583, Seq. ID No. 6585, Seq. ID No. 6591, Seq. ID No. 6595, Seq. ID No. 6605, Seq. ID No. 6607, Seq. ID No. 6611, Seq. ID No. 6615, Seq. ID No. 6617, Seq. ID No. 6619, Seq. ID No. 6623, Seq. ID No. 6625, Seq. ID No. 6629, Seq. ID No. 6633, Seq. ID No. 6635, Seq. ID No. 6641, and Seq. ID No. 6647.

In another preferred embodiment, the invention comprises an isolated polynucleotide of the Alloiococcus otitidis genomic sequence encoding a polypeptide, which has been identified by the Applicants' assignee's ORF finder program that searches for an ATG, GTG or TTG Start codon between “Stop-Stop” region, and the polynucleotide is selected from one of Seq. ID No. 1, Seq. ID No. 5, Seq. ID No. 11, Seq. ID No. 13, Seq. ID No. 15, Seq. ID No. 17, Seq. ID No. 19, Seq. ID No. 21, Seq. ID No. 23, Seq. ID No. 27, Seq. ID No. 29, Seq. ID No. 31, Seq. ID No. 35, Seq. ID No. 37, Seq. ID No. 41, Seq. ID No. 43, Seq. ID No. 45, Seq. ID No. 47, Seq. ID No. 49, Seq. ID No. 51, Seq. ID No. 53, Seq. ID No. 55, Seq. ID No. 57, Seq. ID No. 59, Seq. ID No. 65, Seq. ID No. 67, Seq. ID No. 75, Seq. ID No. 77, Seq. ID No. 79, Seq. ID No. 81, Seq. ID No. 83, Seq. ID No. 89, Seq. ID No. 91, Seq. ID No. 93, Seq. ID No. 97, Seq. ID No. 99, Seq. ID No. 103, Seq. ID No. 105, Seq. ID No. 107, Seq. ID No. 109, Seq. ID No. 111, Seq. ID No. 113, Seq. ID No. 115, Seq. ID No. 117, Seq. ID No. 119, Seq. ID No. 121, Seq. ID No. 127, Seq. ID No. 129, Seq. ID No. 131, Seq. ID No. 133, Seq. ID No. 135, Seq. ID No. 137, Seq. ID No. 139, Seq. ID No. 143, Seq. ID No. 145, Seq. ID No. 147, Seq. ID No. 149, Seq. ID No. 153, Seq. ID No. 155, Seq. ID No. 157, Seq. ID No. 159, Seq. ID No. 161, Seq. ID No. 163, Seq. ID No. 165, Seq. ID No. 167, Seq. ID No. 169, Seq. ID No. 171, Seq. ID No. 175, Seq. ID No. 177, Seq. ID No. 179, Seq. ID No. 181, Seq. ID No. 183, Seq. ID No. 187, Seq. ID No. 191, Seq. ID No. 193, Seq. ID No. 195, Seq. ID No. 201, Seq. ID No. 205, Seq. ID No. 207, Seq. ID No. 215, Seq. ID No. 219, Seq. ID No. 221, Seq. ID No. 223, Seq. ID No. 225, Seq. ID No. 227, Seq. ID No. 231, Seq. ID No. 233, Seq. ID No. 239, Seq. ID No. 243, Seq. ID No. 245, Seq. ID No. 247, Seq. ID No. 253, Seq. ID No. 257, Seq. ID No. 259, Seq. ID No. 261, Seq. ID No. 263, Seq. ID No. 265, Seq. ID No. 267, Seq. ID No. 269, Seq. ID No. 273, Seq. ID No. 277, Seq. ID No. 279, Seq. ID No. 281, Seq. ID No. 283, Seq. ID No. 291, Seq. ID No. 293, Seq. ID No. 297, Seq. ID No. 301, Seq. ID No. 305, Seq. ID No. 307, Seq. ID No. 309, Seq. ID No. 313, Seq. ID No. 317, Seq. ID No. 321, Seq. ID No. 323, Seq. ID No. 325, Seq. ID No. 331, Seq. ID No. 333, Seq. ID No. 335, Seq. ID No. 337, Seq. ID No. 339, Seq. ID No. 343, Seq. ID No. 345, Seq. ID No. 347, Seq. ID No. 349, Seq. ID No. 353, Seq. ID No. 355, Seq. ID No. 357, Seq. ID No. 359, Seq. ID No. 361, Seq. ID No. 363, Seq. ID No. 365, Seq. ID No. 367, Seq. ID No. 369, Seq. ID No. 371, Seq. ID No. 375, Seq. ID No. 377, Seq. ID No. 379, Seq. ID No. 383, Seq. ID No. 385, Seq. ID No. 387, Seq. ID No. 389, Seq. ID No. 391, Seq. ID No. 393, Seq. ID No. 397, Seq. ID No. 401, Seq. ID No. 403, Seq. ID No. 405, Seq. ID No. 407, Seq. ID No. 409, Seq. ID No. 415, Seq. ID No. 423, Seq. ID No. 425, Seq. ID No. 427, Seq. ID No. 429, Seq. ID No. 431, Seq. ID No. 433, Seq. ID No. 435, Seq. ID No. 437, Seq. ID No. 439, Seq. ID No. 441, Seq. ID No. 443, Seq. ID No. 445, Seq. ID No. 447, Seq. ID No. 451, Seq. ID No. 453, Seq. ID No. 455, Seq. ID No. 457, Seq. ID No. 461, Seq. ID No. 463, Seq. ID No. 467, Seq. ID No. 469, Seq. ID No. 471, Seq. ID No. 473, Seq. ID No. 475, Seq. ID No. 477, Seq. ID No. 479, Seq. ID No. 481, Seq. ID No. 483, Seq. ID No. 485, Seq. ID No. 489, Seq. ID No. 491, Seq. ID No. 493, Seq. ID No. 495, Seq. ID No. 501, Seq. ID No. 505, Seq. ID No. 507, Seq. ID No. 511, Seq. ID No. 515, Seq. ID No. 517, Seq. ID No. 523, Seq. ID No. 529, Seq. ID No. 531, Seq. ID No. 533, Seq. ID No. 537, Seq. ID No. 539, Seq. ID No. 543, Seq. ID No. 547, Seq. ID No. 549, Seq. ID No. 551, Seq. ID No. 553, Seq. ID No. 555, Seq. ID No. 557, Seq. ID No. 559, Seq. ID No. 561, Seq. ID No. 565, Seq. ID No. 569, Seq. ID No. 571, Seq. ID No. 573, Seq. ID No. 575, Seq. ID No. 579, Seq. ID No. 581, Seq. ID No. 585, Seq. ID No. 587, Seq. ID No. 589, Seq. ID No. 591, Seq. ID No. 593, Seq. ID No. 595, Seq. ID No. 597, Seq. ID No. 599, Seq. ID No. 601, Seq. ID No. 603, Seq. ID No. 605, Seq. ID No. 609, Seq. ID No. 613, Seq. ID No. 615, Seq. ID No. 617, Seq. ID No. 619, Seq. ID No. 621, Seq. ID No. 623, Seq. ID No. 625, Seq. ID No. 627, Seq. ID No. 635, Seq. ID No. 637, Seq. ID No. 643, Seq. ID No. 645, Seq. ID No. 647, Seq. ID No. 649, Seq. ID No. 655, Seq. ID No. 663, Seq. ID No. 665, Seq. ID No. 667, Seq. ID No. 669, Seq. ID No. 675, Seq. ID No. 681, Seq. ID No. 683, Seq. ID No. 687, Seq. ID No. 689, Seq. ID No. 691, Seq. ID No. 695, Seq. ID No. 697, Seq. ID No. 699, Seq. ID No. 701, Seq. ID No. 703, Seq. ID No. 705, Seq. ID No. 707, Seq. ID No. 709, Seq. ID No. 713, Seq. ID No. 725, Seq. ID No. 727, Seq. ID No. 729, Seq. ID No. 731, Seq. ID No. 733, Seq. ID No. 735, Seq. ID No. 739, Seq. ID No. 741, Seq. ID No. 745, Seq. ID No. 747, Seq. ID No. 749, Seq. ID No. 751, Seq. ID No. 753, Seq. ID No. 759, Seq. ID No. 763, Seq. ID No. 765, Seq. ID No. 773, Seq. ID No. 775, Seq. ID No. 781, Seq. ID No. 783, Seq. ID No. 787, Seq. ID No. 793, Seq. ID No. 795, Seq. ID No. 799, Seq. ID No. 801, Seq. ID No. 805, Seq. ID No. 807, Seq. ID No. 809, Seq. ID No. 811, Seq. ID No. 813, Seq. ID No. 815, Seq. ID No. 817, Seq. ID No. 819, Seq. ID No. 821, Seq. ID No. 823, Seq. ID No. 827, Seq. ID No. 829, Seq. ID No. 831, Seq. ID No. 835, Seq. ID No. 837, Seq. ID No. 839, Seq. ID No. 843, Seq. ID No. 845, Seq. ID No. 847, Seq. ID No. 855, Seq. ID No. 859, Seq. ID No. 861, Seq. ID No. 865, Seq. ID No. 867, Seq. ID No. 871, Seq. ID No. 873, Seq. ID No. 875, Seq. ID No. 877, Seq. ID No. 879, Seq. ID No. 881, Seq. ID No. 885, Seq. ID No. 895, Seq. ID No. 897, Seq. ID No. 899, Seq. ID No. 901, Seq. ID No. 903, Seq. ID No. 905, Seq. ID No. 907, Seq. ID No. 909, Seq. ID No. 913, Seq. ID No. 915, Seq. ID No. 921, Seq. ID No. 923, Seq. ID No. 925, Seq. ID No. 931, Seq. ID No. 933, Seq. ID No. 935, Seq. ID No. 937, Seq. ID No. 939, Seq. ID No. 941, Seq. ID No. 943, Seq. ID No. 947, Seq. ID No. 949, Seq. ID No. 951, Seq. ID No. 953, Seq. ID No. 955, Seq. ID No. 957, Seq. ID No. 961, Seq. ID No. 963, Seq. ID No. 965, Seq. ID No. 967, Seq. ID No. 969, Seq. ID No. 971, Seq. ID No. 973, Seq. ID No. 975, Seq. ID No. 981, Seq. ID No. 983, Seq. ID No. 985, Seq. ID No. 987, Seq. ID No. 989, Seq. ID No. 991, Seq. ID No. 993, Seq. ID No. 995, Seq. ID No. 997, Seq. ID No. 1001, Seq. ID No. 1003, Seq. ID No. 1005, Seq. ID No. 1007, Seq. ID No. 1009, Seq. ID No. 1017, Seq. ID No. 1019, Seq. ID No. 1023, Seq. ID No. 1025, Seq. ID No. 1027, Seq. ID No. 1039, Seq. ID No. 1041, Seq. ID No. 1043, Seq. ID No. 1045, Seq. ID No. 1047, Seq. ID No. 1049, Seq. ID No. 1051, Seq. ID No. 1057, Seq. ID No. 1059, Seq. ID No. 1061, Seq. ID No. 1065, Seq. ID No. 1069, Seq. ID No. 1075, Seq. ID No. 1077, Seq. ID No. 1079, Seq. ID No. 1081, Seq. ID No. 1083, Seq. ID No. 1085, Seq. ID No. 1087, Seq. ID No. 1091, Seq. ID No. 1093, Seq. ID No. 1095, Seq. ID No. 1097, Seq. ID No. 1099, Seq. ID No. 1101, Seq. ID No. 1103, Seq. ID No. 1107, Seq. ID No. 1109, Seq. ID No. 1111, Seq. ID No. 1113, Seq. ID No. 1115, Seq. ID No. 1119, Seq. ID No. 1121, Seq. ID No. 1123, Seq. ID No. 1125, Seq. ID No. 1127, Seq. ID No. 1129, Seq. ID No. 1131, Seq. ID No. 1137, Seq. ID No. 1139, Seq. ID No. 1141, Seq. ID No. 1143, Seq. ID No. 1145, Seq. ID No. 1147, Seq. ID No. 1149, Seq. ID No. 1151, Seq. ID No. 1153, Seq. ID No. 1155, Seq. ID No. 1157, Seq. ID No. 1159, Seq. ID No. 1161, Seq. ID No. 1163, Seq. ID No. 1171, Seq. ID No. 1175, Seq. ID No. 1177, Seq. ID No. 1179, Seq. ID No. 1181, Seq. ID No. 1187, Seq. ID No. 1191, Seq. ID No. 1193, Seq. ID No. 1195, Seq. ID No. 1197, Seq. ID No. 1201, Seq. ID No. 1203, Seq. ID No. 1205, Seq. ID No. 1207, Seq. ID No. 1211, Seq. ID No. 1213, Seq. ID No. 1215, Seq. ID No. 1217, Seq. ID No. 1221, Seq. ID No. 1223, Seq. ID No. 1225, Seq. ID No. 1231, Seq. ID No. 1233, Seq. ID No. 1237, Seq. ID No. 1239, Seq. ID No. 1241, Seq. ID No. 1245, Seq. ID No. 1247, Seq. ID No. 1249, Seq. ID No. 1251, Seq. ID No. 1253, Seq. ID No. 1255, Seq. ID No. 1257, Seq. ID No. 1259, Seq. ID No. 1261, Seq. ID No. 1263, Seq. ID No. 1271, Seq. ID No. 1273, Seq. ID No. 1275, Seq. ID No. 1277, Seq. ID No. 1279, Seq. ID No. 1285, Seq. ID No. 1289, Seq. ID No. 1291, Seq. ID No. 1293, Seq. ID No. 1295, Seq. ID No. 1297, Seq. ID No. 1299, Seq. ID No. 1301, Seq. ID No. 1303, Seq. ID No. 1305, Seq. ID No. 1307, Seq. ID No. 1309, Seq. ID No. 1311, Seq. ID No. 1313, Seq. ID No. 1317, Seq. ID No. 1319, Seq. ID No. 1321, Seq. ID No. 1331, Seq. ID No. 1333, Seq. ID No. 1335, Seq. ID No. 1337, Seq. ID No. 1339, Seq. ID No. 1343, Seq. ID No. 1345, Seq. ID No. 1347, Seq. ID No. 1349, Seq. ID No. 1351, Seq. ID No. 1359, Seq. ID No. 1361, Seq. ID No. 1363, Seq. ID No. 1365, Seq. ID No. 1369, Seq. ID No. 1371, Seq. ID No. 1375, Seq. ID No. 1377, Seq. ID No. 1381, Seq. ID No. 1383, Seq. ID No. 1385, Seq. ID No. 1387, Seq. ID No. 1389, Seq. ID No. 1391, Seq. ID No. 1393, Seq. ID No. 1401, Seq. ID No. 1403, Seq. ID No. 1407, Seq. ID No. 1409, Seq. ID No. 1411, Seq. ID No. 1413, Seq. ID No. 1415, Seq. ID No. 1419, Seq. ID No. 1421, Seq. ID No. 1423, Seq. ID No. 1425, Seq. ID No. 1427, Seq. ID No. 1429, Seq. ID No. 1433, Seq. ID No. 1437, Seq. ID No. 1441, Seq. ID No. 1443, Seq. ID No. 1447, Seq. ID No. 1449, Seq. ID No. 1451, Seq. ID No. 1455, Seq. ID No. 1459, Seq. ID No. 1463, Seq. ID No. 1465, Seq. ID No. 1467, Seq. ID No. 1469, Seq. ID No. 1471, Seq. ID No. 1473, Seq. ID No. 1475, Seq. ID No. 1477, Seq. ID No. 1479, Seq. ID No. 1483, Seq. ID No. 1485, Seq. ID No. 1487, Seq. ID No. 1491, Seq. ID No. 1493, Seq. ID No. 1495, Seq. ID No. 1497, Seq. ID No. 1499, Seq. ID No. 1501, Seq. ID No. 1505, Seq. ID No. 1507, Seq. ID No. 1511, Seq. ID No. 1517, Seq. ID No. 1519, Seq. ID No. 1523, Seq. ID No. 1525, Seq. ID No. 1527, Seq. ID No. 1531, Seq. ID No. 1539, Seq. ID No. 1543, Seq. ID No. 1549, Seq. ID No. 1551, Seq. ID No. 1553, Seq. ID No. 1555, Seq. ID No. 1557, Seq. ID No. 1561, Seq. ID No. 1563, Seq. ID No. 1565, Seq. ID No. 1567, Seq. ID No. 1571, Seq. ID No. 1573, Seq. ID No. 1575, Seq. ID No. 1577, Seq. ID No. 1579, Seq. ID No. 1581, Seq. ID No. 1589, Seq. ID No. 1593, Seq. ID No. 1595, Seq. ID No. 1597, Seq. ID No. 1599, Seq. ID No. 1601, Seq. ID No. 1607, Seq. ID No. 1611, Seq. ID No. 1613, Seq. ID No. 1615, Seq. ID No. 1617, Seq. ID No. 1619, Seq. ID No. 1623, Seq. ID No. 1625, Seq. ID No. 1627, Seq. ID No. 1633, Seq. ID No. 1637, Seq. ID No. 1639, Seq. ID No. 1641, Seq. ID No. 1643, Seq. ID No. 1647, Seq. ID No. 1651, Seq. ID No. 1653, Seq. ID No. 1655, Seq. ID No. 1657, Seq. ID No. 1661, Seq. ID No. 1663, Seq. ID No. 1665, Seq. ID No. 1669, Seq. ID No. 1675, Seq. ID No. 1679, Seq. ID No. 1681, Seq. ID No. 1685, Seq. ID No. 1687, Seq. ID No. 1689, Seq. ID No. 1691, Seq. ID No. 1693, Seq. ID No. 1695, Seq. ID No. 1697, Seq. ID No. 1699, Seq. ID No. 1703, Seq. ID No. 1705, Seq. ID No. 1707, Seq. ID No. 1711, Seq. ID No. 1713, Seq. ID No. 1715, Seq. ID No. 1717, Seq. ID No. 1719, Seq. ID No. 1721, Seq. ID No. 1723, Seq. ID No. 1727, Seq. ID No. 1729, Seq. ID No. 1735, Seq. ID No. 1737, Seq. ID No. 1739, Seq. ID No. 1741, Seq. ID No. 1747, Seq. ID No. 1749, Seq. ID No. 1751, Seq. ID No. 1755, Seq. ID No. 1757, Seq. ID No. 1759, Seq. ID No. 1763, Seq. ID No. 1769, Seq. ID No. 1771, Seq. ID No. 1775, Seq. ID No. 1777, Seq. ID No. 1779, Seq. ID No. 1781, Seq. ID No. 1783, Seq. ID No. 1785, Seq. ID No. 1789, Seq. ID No. 1791, Seq. ID No. 1793, Seq. ID No. 1795, Seq. ID No. 1797, Seq. ID No. 1805, Seq. ID No. 1809, Seq. ID No. 1811, Seq. ID No. 1813, Seq. ID No. 1815, Seq. ID No. 1817, Seq. ID No. 1819, Seq. ID No. 1821, Seq. ID No. 1823, Seq. ID No. 1825, Seq. ID No. 1829, Seq. ID No. 1833, Seq. ID No. 1841, Seq. ID No. 1843, Seq. ID No. 1845, Seq. ID No. 1847, Seq. ID No. 1849, Seq. ID No. 1853, Seq. ID No. 1855, Seq. ID No. 1857, Seq. ID No. 1859, Seq. ID No. 1861, Seq. ID No. 1863, Seq. ID No. 1865, Seq. ID No. 1867, Seq. ID No. 1871, Seq. ID No. 1873, Seq. ID No. 1875, Seq. ID No. 1877, Seq. ID No. 1881, Seq. ID No. 1883, Seq. ID No. 1885, Seq. ID No. 1887, Seq. ID No. 1889, Seq. ID No. 1895, Seq. ID No. 1903, Seq. ID No. 1905, Seq. ID No. 1907, Seq. ID No. 1911, Seq. ID No. 1913, Seq. ID No. 1915, Seq. ID No. 1919, Seq. ID No. 1923, Seq. ID No. 1925, Seq. ID No. 1927, Seq. ID No. 1929, Seq. ID No. 1931, Seq. ID No. 1935, Seq. ID No. 1941, Seq. ID No. 1943, Seq. ID No. 1945, Seq. ID No. 1947, Seq. ID No. 1949, Seq. ID No. 1951, Seq. ID No. 1953, Seq. ID No. 1959, Seq. ID No. 1961, Seq. ID No. 1963, Seq. ID No. 1965, Seq. ID No. 1967, Seq. ID No. 1969, Seq. ID No. 1971, Seq. ID No. 1979, Seq. ID No. 1983, Seq. ID No. 1985, Seq. ID No. 1987, Seq. ID No. 1989, Seq. ID No. 1991, Seq. ID No. 1995, Seq. ID No. 1997, Seq. ID No. 1999, Seq. ID No. 2001, Seq. ID No. 2003, Seq. ID No. 2005, Seq. ID No. 2007, Seq. ID No. 2015, Seq. ID No. 2019, Seq. ID No. 2021, Seq. ID No. 2025, Seq. ID No. 2027, Seq. ID No. 2029, Seq. ID No. 2031, Seq. ID No. 2035, Seq. ID No. 2037, Seq. ID No. 2041, Seq. ID No. 2043, Seq. ID No. 2045, Seq. ID No. 2047, Seq. ID No. 2053, Seq. ID No. 2055, Seq. ID No. 2059, Seq. ID No. 2063, Seq. ID No. 2065, Seq. ID No. 2067, Seq. ID No. 2069, Seq. ID No. 2071, Seq. ID No. 2073, Seq. ID No. 2075, Seq. ID No. 2077, Seq. ID No. 2079, Seq. ID No. 2081, Seq. ID No. 2083, Seq. ID No. 2085, Seq. ID No. 2089, Seq. ID No. 2091, Seq. ID No. 2093, Seq. ID No. 2097, Seq. ID No. 2099, Seq. ID No. 2101, Seq. ID No. 2103, Seq. ID No. 2107, Seq. ID No. 2109, Seq. ID No. 2111, Seq. ID No. 2115, Seq. ID No. 2117, Seq. ID No. 2119, Seq. ID No. 2125, Seq. ID No. 2127, Seq. ID No. 2129, Seq. ID No. 2131, Seq. ID No. 2133, Seq. ID No. 2135, Seq. ID No. 2137, Seq. ID No. 2141, Seq. ID No. 2143, Seq. ID No. 2145, Seq. ID No. 2149, Seq. ID No. 2151, Seq. ID No. 2153, Seq. ID No. 2155, Seq. ID No. 2157, Seq. ID No. 2159, Seq. ID No. 2161, Seq. ID No. 2165, Seq. ID No. 2167, Seq. ID No. 2169, Seq. ID No. 2179, Seq. ID No. 2181, Seq. ID No. 2183, Seq. ID No. 2189, Seq. ID No. 2191, Seq. ID No. 2195, Seq. ID No. 2197, Seq. ID No. 2199, Seq. ID No. 2201, Seq. ID No. 2203, Seq. ID No. 2205, Seq. ID No. 2207, Seq. ID No. 2209, Seq. ID No. 2211, Seq. ID No. 2213, Seq. ID No. 2215, Seq. ID No. 2217, Seq. ID No. 2221, Seq. ID No. 2223, Seq. ID No. 2227, Seq. ID No. 2229, Seq. ID No. 2235, Seq. ID No. 2241, Seq. ID No. 2245, Seq. ID No. 2247, Seq. ID No. 2251, Seq. ID No. 2253, Seq. ID No. 2257, Seq. ID No. 2259, Seq. ID No. 2261, Seq. ID No. 2263, Seq. ID No. 2265, Seq. ID No. 2267, Seq. ID No. 2269, Seq. ID No. 2275, Seq. ID No. 2277, Seq. ID No. 2279, Seq. ID No. 2285, Seq. ID No. 2287, Seq. ID No. 2297, Seq. ID No. 2299, Seq. ID No. 2301, Seq. ID No. 2309, Seq. ID No. 2311, Seq. ID No. 2315, Seq. ID No. 2327, Seq. ID No. 2329, Seq. ID No. 2333, Seq. ID No. 2343, Seq. ID No. 2345, Seq. ID No. 2349, Seq. ID No. 2351, Seq. ID No. 2357, Seq. ID No. 2363, Seq. ID No. 2365, Seq. ID No. 2367, Seq. ID No. 2369, Seq. ID No. 2371, Seq. ID No. 2373, Seq. ID No. 2375, Seq. ID No. 2379, Seq. ID No. 2381, Seq. ID No. 2385, Seq. ID No. 2387, Seq. ID No. 2391, Seq. ID No. 2393, Seq. ID No. 2395, Seq. ID No. 2397, Seq. ID No. 2399, Seq. ID No. 2401, Seq. ID No. 2405, Seq. ID No. 2409, Seq. ID No. 2413, Seq. ID No. 2415, Seq. ID No. 2417, Seq. ID No. 2421, Seq. ID No. 2423, Seq. ID No. 2425, Seq. ID No. 2427, Seq. ID No. 2429, Seq. ID No. 2435, Seq. ID No. 2437, Seq. ID No. 2445, Seq. ID No. 2447, Seq. ID No. 2449, Seq. ID No. 2451, Seq. ID No. 2453, Seq. ID No. 2455, Seq. ID No. 2457, Seq. ID No. 2459, Seq. ID No. 2461, Seq. ID No. 2463, Seq. ID No. 2465, Seq. ID No. 2469, Seq. ID No. 2471, Seq. ID No. 2475, Seq. ID No. 2481, Seq. ID No. 2483, Seq. ID No. 2485, Seq. ID No. 2491, Seq. ID No. 2493, Seq. ID No. 2495, Seq. ID No. 2499, Seq. ID No. 2501, Seq. ID No. 2503, Seq. ID No. 2505, Seq. ID No. 2507, Seq. ID No. 2511, Seq. ID No. 2513, Seq. ID No. 2517, Seq. ID No. 2519, Seq. ID No. 2521, Seq. ID No. 2525, Seq. ID No. 2529, Seq. ID No. 2533, Seq. ID No. 2535, Seq. ID No. 2537, Seq. ID No. 2539, Seq. ID No. 2547, Seq. ID No. 2549, Seq. ID No. 2551, Seq. ID No. 2553, Seq. ID No. 2559, Seq. ID No. 2561, Seq. ID No. 2563, Seq. ID No. 2565, Seq. ID No. 2573, Seq. ID No. 2575, Seq. ID No. 2577, Seq. ID No. 2579, Seq. ID No. 2581, Seq. ID No. 2587, Seq. ID No. 2589, Seq. ID No. 2591, Seq. ID No. 2593, Seq. ID No. 2595, Seq. ID No. 2599, Seq. ID No. 2603, Seq. ID No. 2605, Seq. ID No. 2607, Seq. ID No. 2609, Seq. ID No. 2611, Seq. ID No. 2613, Seq. ID No. 2617, Seq. ID No. 2619, Seq. ID No. 2623, Seq. ID No. 2625, Seq. ID No. 2627, Seq. ID No. 2629, Seq. ID No. 2631, Seq. ID No. 2633, Seq. ID No. 2635, Seq. ID No. 2637, Seq. ID No. 2639, Seq. ID No. 2641, Seq. ID No. 2643, Seq. ID No. 2645, Seq. ID No. 2647, Seq. ID No. 2649, Seq. ID No. 2651, Seq. ID No. 2653, Seq. ID No. 2655, Seq. ID No. 2659, Seq. ID No. 2663, Seq. ID No. 2667, Seq. ID No. 2669, Seq. ID No. 2671, Seq. ID No. 2675, Seq. ID No. 2677, Seq. ID No. 2683, Seq. ID No. 2685, Seq. ID No. 2687, Seq. ID No. 2689, Seq. ID No. 2691, Seq. ID No. 2693, Seq. ID No. 2695, Seq. ID No. 2697, Seq. ID No. 2699, Seq. ID No. 2703, Seq. ID No. 2709, Seq. ID No. 2711, Seq. ID No. 2715, Seq. ID No. 2717, Seq. ID No. 2719, Seq. ID No. 2723, Seq. ID No. 2725, Seq. ID No. 2727, Seq. ID No. 2729, Seq. ID No. 2731, Seq. ID No. 2735, Seq. ID No. 2737, Seq. ID No. 2739, Seq. ID No. 2749, Seq. ID No. 2751, Seq. ID No. 2753, Seq. ID No. 2755, Seq. ID No. 2759, Seq. ID No. 2765, Seq. ID No. 2767, Seq. ID No. 2769, Seq. ID No. 2771, Seq. ID No. 2773, Seq. ID No. 2775, Seq. ID No. 2779, Seq. ID No. 2781, Seq. ID No. 2783, Seq. ID No. 2787, Seq. ID No. 2789, Seq. ID No. 2791, Seq. ID No. 2793, Seq. ID No. 2795, Seq. ID No. 2797, Seq. ID No. 2799, Seq. ID No. 2801, Seq. ID No. 2803, Seq. ID No. 2807, Seq. ID No. 2809, Seq. ID No. 2813, Seq. ID No. 2815, Seq. ID No. 2821, Seq. ID No. 2823, Seq. ID No. 2827, Seq. ID No. 2829, Seq. ID No. 2831, Seq. ID No. 2833, Seq. ID No. 2839, Seq. ID No. 2841, Seq. ID No. 2843, Seq. ID No. 2845, Seq. ID No. 2847, Seq. ID No. 2855, Seq. ID No. 2861, Seq. ID No. 2863, Seq. ID No. 2865, Seq. ID No. 2867, Seq. ID No. 2869, Seq. ID No. 2871, Seq. ID No. 2877, Seq. ID No. 2881, Seq. ID No. 2883, Seq. ID No. 2885, Seq. ID No. 2887, Seq. ID No. 2891, Seq. ID No. 2893, Seq. ID No. 2895, Seq. ID No. 2899, Seq. ID No. 2901, Seq. ID No. 2907, Seq. ID No. 2915, Seq. ID No. 2917, Seq. ID No. 2919, Seq. ID No. 2921, Seq. ID No. 2923, Seq. ID No. 2927, Seq. ID No. 2931, Seq. ID No. 2933, Seq. ID No. 2935, Seq. ID No. 2943, Seq. ID No. 2945, Seq. ID No. 2951, Seq. ID No. 2953, Seq. ID No. 2957, Seq. ID No. 2959, Seq. ID No. 2961, Seq. ID No. 2963, Seq. ID No. 2965, Seq. ID No. 2967, Seq. ID No. 2971, Seq. ID No. 2973, Seq. ID No. 2975, Seq. ID No. 2977, Seq. ID No. 2979, Seq. ID No. 2983, Seq. ID No. 2985, Seq. ID No. 2987, Seq. ID No. 2991, Seq. ID No. 2993, Seq. ID No. 2999, Seq. ID No. 3001, Seq. ID No. 3005, Seq. ID No. 3009, Seq. ID No. 3011, Seq. ID No. 3015, Seq. ID No. 3017, Seq. ID No. 3019, Seq. ID No. 3021, Seq. ID No. 3027, Seq. ID No. 3033, Seq. ID No. 3035, Seq. ID No. 3037, Seq. ID No. 3039, Seq. ID No. 3043, Seq. ID No. 3045, Seq. ID No. 3047, Seq. ID No. 3049, Seq. ID No. 3051, Seq. ID No. 3053, Seq. ID No. 3055, Seq. ID No. 3057, Seq. ID No. 3059, Seq. ID No. 3067, Seq. ID No. 3069, Seq. ID No. 3071, Seq. ID No. 3079, Seq. ID No. 3083, Seq. ID No. 3085, Seq. ID No. 3087, Seq. ID No. 3091, Seq. ID No. 3093, Seq. ID No. 3099, Seq. ID No. 3101, Seq. ID No. 3103, Seq. ID No. 3109, Seq. ID No. 3111, Seq. ID No. 3115, Seq. ID No. 3117, Seq. ID No. 3119, Seq. ID No. 3121, Seq. ID No. 3123, Seq. ID No. 3125, Seq. ID No. 3129, Seq. ID No. 3131, Seq. ID No. 3133, Seq. ID No. 3135, Seq. ID No. 3137, Seq. ID No. 3139, Seq. ID No. 3141, Seq. ID No. 3143, Seq. ID No. 3147, Seq. ID No. 3149, Seq. ID No. 3151, Seq. ID No. 3153, Seq. ID No. 3155, Seq. ID No. 3157, Seq. ID No. 3159, Seq. ID No. 3161, Seq. ID No. 3165, Seq. ID No. 3167, Seq. ID No. 3169, Seq. ID No. 3171, Seq. ID No. 3173, Seq. ID No. 3179, Seq. ID No. 3181, Seq. ID No. 3183, Seq. ID No. 3185, Seq. ID No. 3187, Seq. ID No. 3189, Seq. ID No. 3191, Seq. ID No. 3193, Seq. ID No. 3195, Seq. ID No. 3197, Seq. ID No. 3203, Seq. ID No. 3205, Seq. ID No. 3207, Seq. ID No. 3209, Seq. ID No. 3211, Seq. ID No. 3217, Seq. ID No. 3223, Seq. ID No. 3225, Seq. ID No. 3227, Seq. ID No. 3229, Seq. ID No. 3235, Seq. ID No. 3249, Seq. ID No. 3251, Seq. ID No. 3253, Seq. ID No. 3255, Seq. ID No. 3257, Seq. ID No. 3259, Seq. ID No. 3263, Seq. ID No. 3265, Seq. ID No. 3269, Seq. ID No. 3271, Seq. ID No. 3273, Seq. ID No. 3277, Seq. ID No. 3281, Seq. ID No. 3283, Seq. ID No. 3285, Seq. ID No. 3289, Seq. ID No. 3291, Seq. ID No. 3293, Seq. ID No. 3299, Seq. ID No. 3301, Seq. ID No. 3303, Seq. ID No. 3305, Seq. ID No. 3307, Seq. ID No. 3309, Seq. ID No. 3311, Seq. ID No. 3313, Seq. ID No. 3315, Seq. ID No. 3319, Seq. ID No. 3321, Seq. ID No. 3323, Seq. ID No. 3325, Seq. ID No. 3331, Seq. ID No. 3335, Seq. ID No. 3337, Seq. ID No. 3339, Seq. ID No. 3341, Seq. ID No. 3345, Seq. ID No. 3347, Seq. ID No. 3349, Seq. ID No. 3353, Seq. ID No. 3355, Seq. ID No. 3359, Seq. ID No. 3361, Seq. ID No. 3363, Seq. ID No. 3365, Seq. ID No. 3367, Seq. ID No. 3369, Seq. ID No. 3371, Seq. ID No. 3373, Seq. ID No. 3375, Seq. ID No. 3377, Seq. ID No. 3379, Seq. ID No. 3381, Seq. ID No. 3383, Seq. ID No. 3397, Seq. ID No. 3399, Seq. ID No. 3401, Seq. ID No. 3407, Seq. ID No. 3409, Seq. ID No. 3413, Seq. ID No. 3415, Seq. ID No. 3419, Seq. ID No. 3425, Seq. ID No. 3427, Seq. ID No. 3431, Seq. ID No. 3433, Seq. ID No. 3435, Seq. ID No. 3439, Seq. ID No. 3441, Seq. ID No. 3443, Seq. ID No. 3445, Seq. ID No. 3447, Seq. ID No. 3455, Seq. ID No. 3457, Seq. ID No. 3459, Seq. ID No. 3461, Seq. ID No. 3465, Seq. ID No. 3467, Seq. ID No. 3471, Seq. ID No. 3473, Seq. ID No. 3475, Seq. ID No. 3479, Seq. ID No. 3483, Seq. ID No. 3485, Seq. ID No. 3487, Seq. ID No. 3489, Seq. ID No. 3495, Seq. ID No. 3497, Seq. ID No. 3499, Seq. ID No. 3501, Seq. ID No. 3505, Seq. ID No. 3507, Seq. ID No. 3509, Seq. ID No. 3511, Seq. ID No. 3513, Seq. ID No. 3517, Seq. ID No. 3519, Seq. ID No. 3521, Seq. ID No. 3525, Seq. ID No. 3529, Seq. ID No. 3531, Seq. ID No. 3533, Seq. ID No. 3535, Seq. ID No. 3537, Seq. ID No. 3539, Seq. ID No. 3541, Seq. ID No. 3543, Seq. ID No. 3545, Seq. ID No. 3547, Seq. ID No. 3549, Seq. ID No. 3551, Seq. ID No. 3553, Seq. ID No. 3555, Seq. ID No. 3557, Seq. ID No. 3559, Seq. ID No. 3561, Seq. ID No. 3563, Seq. ID No. 3565, Seq. ID No. 3567, Seq. ID No. 3569, Seq. ID No. 3571, Seq. ID No. 3573, Seq. ID No. 3575, Seq. ID No. 3579, Seq. ID No. 3581, Seq. ID No. 3583, Seq. ID No. 3587, Seq. ID No. 3589, Seq. ID No. 3595, Seq. ID No. 3597, Seq. ID No. 3599, Seq. ID No. 3603, Seq. ID No. 3605, Seq. ID No. 3607, Seq. ID No. 3609, Seq. ID No. 3611, Seq. ID No. 3613, Seq. ID No. 3615, Seq. ID No. 3617, Seq. ID No. 3619, Seq. ID No. 3621, Seq. ID No. 3623, Seq. ID No. 3627, Seq. ID No. 3629, Seq. ID No. 3635, Seq. ID No. 3637, Seq. ID No. 3639, Seq. ID No. 3641, Seq. ID No. 3645, Seq. ID No. 3647, Seq. ID No. 3649, Seq. ID No. 3651, Seq. ID No. 3653, Seq. ID No. 3657, Seq. ID No. 3659, Seq. ID No. 3663, Seq. ID No. 3665, Seq. ID No. 3667, Seq. ID No. 3671, Seq. ID No. 3673, Seq. ID No. 3675, Seq. ID No. 3677, Seq. ID No. 3679, Seq. ID No. 3681, Seq. ID No. 3683, Seq. ID No. 3685, Seq. ID No. 3687, Seq. ID No. 3689, Seq. ID No. 3691, Seq. ID No. 3693, Seq. ID No. 3695, Seq. ID No. 3699, Seq. ID No. 3701, Seq. ID No. 3703, Seq. ID No. 3705, Seq. ID No. 3707, Seq. ID No. 3709, Seq. ID No. 3713, Seq. ID No. 3715, Seq. ID No. 3717, Seq. ID No. 3727, Seq. ID No. 3729, Seq. ID No. 3733, Seq. ID No. 3737, Seq. ID No. 3739, Seq. ID No. 3741, Seq. ID No. 3745, Seq. ID No. 3747, Seq. ID No. 3759, Seq. ID No. 3767, Seq. ID No. 3769, Seq. ID No. 3771, Seq. ID No. 3779, Seq. ID No. 3783, Seq. ID No. 3785, Seq. ID No. 3789, Seq. ID No. 3791, Seq. ID No. 3795, Seq. ID No. 3797, Seq. ID No. 3803, Seq. ID No. 3805, Seq. ID No. 3807, Seq. ID No. 3809, Seq. ID No. 3811, Seq. ID No. 3813, Seq. ID No. 3815, Seq. ID No. 3817, Seq. ID No. 3819, Seq. ID No. 3823, Seq. ID No. 3825, Seq. ID No. 3831, Seq. ID No. 3833, Seq. ID No. 3835, Seq. ID No. 3841, Seq. ID No. 3843, Seq. ID No. 3847, Seq. ID No. 3855, Seq. ID No. 3857, Seq. ID No. 3863, Seq. ID No. 3867, Seq. ID No. 3871, Seq. ID No. 3873, Seq. ID No. 3875, Seq. ID No. 3877, Seq. ID No. 3881, Seq. ID No. 3883, Seq. ID No. 3885, Seq. ID No. 3893, Seq. ID No. 3895, Seq. ID No. 3899, Seq. ID No. 3901, Seq. ID No. 3905, Seq. ID No. 3907, Seq. ID No. 3911, Seq. ID No. 3913, Seq. ID No. 3915, Seq. ID No. 3919, Seq. ID No. 3921, Seq. ID No. 3923, Seq. ID No. 3925, Seq. ID No. 3927, Seq. ID No. 3929, Seq. ID No. 3931, Seq. ID No. 3933, Seq. ID No. 3935, Seq. ID No. 3939, Seq. ID No. 3941, Seq. ID No. 3943, Seq. ID No. 3945, Seq. ID No. 3947, Seq. ID No. 3949, Seq. ID No. 3951, Seq. ID No. 3953, Seq. ID No. 3955, Seq. ID No. 3957, Seq. ID No. 3959, Seq. ID No. 3961, Seq. ID No. 3965, Seq. ID No. 3967, Seq. ID No. 3969, Seq. ID No. 3971, Seq. ID No. 3975, Seq. ID No. 3979, Seq. ID No. 3981, Seq. ID No. 3983, Seq. ID No. 3985, Seq. ID No. 3987, Seq. ID No. 3991, Seq. ID No. 3995, Seq. ID No. 3997, Seq. ID No. 3999, Seq. ID No. 4003, Seq. ID No. 4007, Seq. ID No. 4009, Seq. ID No. 4015, Seq. ID No. 4017, Seq. ID No. 4021, Seq. ID No. 4023, Seq. ID No. 4029, Seq. ID No. 4035, Seq. ID No. 4037, Seq. ID No. 4039, Seq. ID No. 4049, Seq. ID No. 4051, Seq. ID No. 4053, Seq. ID No. 4055, Seq. ID No. 4057, Seq. ID No. 4061, Seq. ID No. 4075, Seq. ID No. 4079, Seq. ID No. 4085, Seq. ID No. 4087, Seq. ID No. 4089, Seq. ID No. 4091, Seq. ID No. 4093, Seq. ID No. 4095, Seq. ID No. 4099, Seq. ID No. 4105, Seq. ID No. 4107, Seq. ID No. 4109, Seq. ID No. 4111, Seq. ID No. 4113, Seq. ID No. 4117, Seq. ID No. 4119, Seq. ID No. 4121, Seq. ID No. 4123, Seq. ID No. 4125, Seq. ID No. 4127, Seq. ID No. 4129, Seq. ID No. 4131, Seq. ID No. 4133, Seq. ID No. 4135, Seq. ID No. 4137, Seq. ID No. 4139, Seq. ID No. 4141, Seq. ID No. 4143, Seq. ID No. 4145, Seq. ID No. 4149, Seq. ID No. 4153, Seq. ID No. 4155, Seq. ID No. 4157, Seq. ID No. 4161, Seq. ID No. 4163, Seq. ID No. 4167, Seq. ID No. 4171, Seq. ID No. 4173, Seq. ID No. 4175, Seq. ID No. 4177, Seq. ID No. 4179, Seq. ID No. 4181, Seq. ID No. 4185, Seq. ID No. 4187, Seq. ID No. 4189, Seq. ID No. 4191, Seq. ID No. 4193, Seq. ID No. 4195, Seq. ID No. 4197, Seq. ID No. 4199, Seq. ID No. 4203, Seq. ID No. 4205, Seq. ID No. 4207, Seq. ID No. 4209, Seq. ID No. 4211, Seq. ID No. 4219, Seq. ID No. 4229, Seq. ID No. 4233, Seq. ID No. 4235, Seq. ID No. 4237, Seq. ID No. 4239, Seq. ID No. 4243, Seq. ID No. 4249, Seq. ID No. 4251, Seq. ID No. 4253, Seq. ID No. 4255, Seq. ID No. 4259, Seq. ID No. 4263, Seq. ID No. 4265, Seq. ID No. 4267, Seq. ID No. 4269, Seq. ID No. 4271, Seq. ID No. 4273, Seq. ID No. 4275, Seq. ID No. 4281, Seq. ID No. 4283, Seq. ID No. 4285, Seq. ID No. 4287, Seq. ID No. 4289, Seq. ID No. 4291, Seq. ID No. 4293, Seq. ID No. 4295, Seq. ID No. 4297, Seq. ID No. 4299, Seq. ID No. 4301, Seq. ID No. 4303, Seq. ID No. 4307, Seq. ID No. 4309, Seq. ID No. 4313, Seq. ID No. 4315, Seq. ID No. 4317, Seq. ID No. 4319, Seq. ID No. 4321, Seq. ID No. 4323, Seq. ID No. 4325, Seq. ID No. 4329, Seq. ID No. 4331, Seq. ID No. 4333, Seq. ID No. 4335, Seq. ID No. 4337, Seq. ID No. 4339, Seq. ID No. 4343, Seq. ID No. 4345, Seq. ID No. 4347, Seq. ID No. 4351, Seq. ID No. 4353, Seq. ID No. 4355, Seq. ID No. 4359, Seq. ID No. 4361, Seq. ID No. 4363, Seq. ID No. 4365, Seq. ID No. 4367, Seq. ID No. 4369, Seq. ID No. 4375, Seq. ID No. 4377, Seq. ID No. 4379, Seq. ID No. 4383, Seq. ID No. 4389, Seq. ID No. 4391, Seq. ID No. 4393, Seq. ID No. 4395, Seq. ID No. 4397, Seq. ID No. 4399, Seq. ID No. 4401, Seq. ID No. 4403, Seq. ID No. 4405, Seq. ID No. 4407, Seq. ID No. 4409, Seq. ID No. 4411, Seq. ID No. 4413, Seq. ID No. 4415, Seq. ID No. 4417, Seq. ID No. 4419, Seq. ID No. 4421, Seq. ID No. 4423, Seq. ID No. 4425, Seq. ID No. 4427, Seq. ID No. 4429, Seq. ID No. 4431, Seq. ID No. 4435, Seq. ID No. 4437, Seq. ID No. 4439, Seq. ID No. 4441, Seq. ID No. 4449, Seq. ID No. 4451, Seq. ID No. 4453, Seq. ID No. 4455, Seq. ID No. 4457, Seq. ID No. 4459, Seq. ID No. 4461, Seq. ID No. 4465, Seq. ID No. 4467, Seq. ID No. 4471, Seq. ID No. 4475, Seq. ID No. 4477, Seq. ID No. 4479, Seq. ID No. 4481, Seq. ID No. 4485, Seq. ID No. 4487, Seq. ID No. 4489, Seq. ID No. 4491, Seq. ID No. 4493, Seq. ID No. 4495, Seq. ID No. 4497, Seq. ID No. 4501, Seq. ID No. 4503, Seq. ID No. 4505, Seq. ID No. 4507, Seq. ID No. 4509, Seq. ID No. 4511, Seq. ID No. 4513, Seq. ID No. 4515, Seq. ID No. 4519, Seq. ID No. 4521, Seq. ID No. 4523, Seq. ID No. 4525, Seq. ID No. 4527, Seq. ID No. 4529, Seq. ID No. 4531, Seq. ID No. 4533, Seq. ID No. 4537, Seq. ID No. 4541, Seq. ID No. 4545, Seq. ID No. 4547, Seq. ID No. 4549, Seq. ID No. 4551, Seq. ID No. 4553, Seq. ID No. 4555, Seq. ID No. 4559, Seq. ID No. 4561, Seq. ID No. 4565, Seq. ID No. 4567, Seq. ID No. 4571, Seq. ID No. 4573, Seq. ID No. 4575, Seq. ID No. 4577, Seq. ID No. 4581, Seq. ID No. 4585, Seq. ID No. 4589, Seq. ID No. 4591, Seq. ID No. 4597, Seq. ID No. 4599, Seq. ID No. 4601, Seq. ID No. 4603, Seq. ID No. 4605, Seq. ID No. 4607, Seq. ID No. 4609, Seq. ID No. 4611, Seq. ID No. 4613, Seq. ID No. 4615, Seq. ID No. 4617, Seq. ID No. 4619, Seq. ID No. 4621, Seq. ID No. 4623, Seq. ID No. 4627, Seq. ID No. 4631, Seq. ID No. 4637, Seq. ID No. 4641, Seq. ID No. 4643, Seq. ID No. 4645, Seq. ID No. 4647, Seq. ID No. 4649, Seq. ID No. 4653, Seq. ID No. 4659, Seq. ID No. 4661, Seq. ID No. 4663, Seq. ID No. 4665, Seq. ID No. 4667, Seq. ID No. 4669, Seq. ID No. 4671, Seq. ID No. 4673, Seq. ID No. 4675, Seq. ID No. 4677, Seq. ID No. 4679, Seq. ID No. 4683, Seq. ID No. 4685, Seq. ID No. 468.7, Seq. ID No. 4691, Seq. ID No. 4693, Seq. ID No. 4695, Seq. ID No. 4699, Seq. ID No. 4701, Seq. ID No. 4703, Seq. ID No. 4705, Seq. ID No. 4713, Seq. ID No. 4717, Seq. ID No. 4719, Seq. ID No. 4721, Seq. ID No. 4727, Seq. ID No. 4729, Seq. ID No. 4733, Seq. ID No. 4735, Seq. ID No. 4737, Seq. ID No. 4739, Seq. ID No. 4741, Seq. ID No. 4743, Seq. ID No. 4747, Seq. ID No. 4749, Seq. ID No. 4751, Seq. ID No. 4755, Seq. ID No. 4757, Seq. ID No. 4763, Seq. ID No. 4765, Seq. ID No. 4767, Seq. ID No. 4769, Seq. ID No. 4773, Seq. ID No. 4775, Seq. ID No. 4777, Seq. ID No. 4779, Seq. ID No. 4781, Seq. ID No. 4787, Seq. ID No. 4789, Seq. ID No. 4791, Seq. ID No. 4793, Seq. ID No. 4795, Seq. ID No. 4797, Seq. ID No. 4801, Seq. ID No. 4803, Seq. ID No. 4805, Seq. ID No. 4809, Seq. ID No. 4811, Seq. ID No. 4813, Seq. ID No. 4815, Seq. ID No. 4817, Seq. ID No. 4819, Seq. ID No. 4825, Seq. ID No. 4827, Seq. ID No. 4829, Seq. ID No. 4831, Seq. ID No. 4833, Seq. ID No. 4837, Seq. ID No. 4839, Seq. ID No. 4841, Seq. ID No. 4843, Seq. ID No. 4845, Seq. ID No. 4849, Seq. ID No. 4851, Seq. ID No. 4853, Seq. ID No. 4855, Seq. ID No. 4861, Seq. ID No. 4863, Seq. ID No. 4867, Seq. ID No. 4875, Seq. ID No. 4877, Seq. ID No. 4879, Seq. ID No. 4881, Seq. ID No. 4883, Seq. ID No. 4889, Seq. ID No. 4891, Seq. ID No. 4893, Seq. ID No. 4895, Seq. ID No. 4897, Seq. ID No. 4899, Seq. ID No. 4901, Seq. ID No. 4903, Seq. ID No. 4905, Seq. ID No. 4907, Seq. ID No. 4909, Seq. ID No. 4911, Seq. ID No. 4913, Seq. ID No. 4915, Seq. ID No. 4917, Seq. ID No. 4919, Seq. ID No. 4921, Seq. ID No. 4923, Seq. ID No. 4927, Seq. ID No. 4931, Seq. ID No. 4935, Seq. ID No. 4937, Seq. ID No. 4939, Seq. ID No. 4941, Seq. ID No. 4943, Seq. ID No. 4945, Seq. ID No. 4947, Seq. ID No. 4949, Seq. ID No. 4951, Seq. ID No. 4953, Seq. ID No. 4957, Seq. ID No. 4959, Seq. ID No. 4961, Seq. ID No. 4963, Seq. ID No. 4973, Seq. ID No. 4975, Seq. ID No. 4977, Seq. ID No. 4979, Seq. ID No. 4981, Seq. ID No. 4983, Seq. ID No. 4985, Seq. ID No. 4987, Seq. ID No. 4989, Seq. ID No. 4991, Seq. ID No. 4993, Seq. ID No. 4995, Seq. ID No. 4997, Seq. ID No. 4999, Seq. ID No. 5003, Seq. ID No. 5005, Seq. ID No. 5007, Seq. ID No. 5009, Seq. ID No. 5011, Seq. ID No. 5013, Seq. ID No. 5017, Seq. ID No. 5019, Seq. ID No. 5021, Seq. ID No. 5023, Seq. ID No. 5025, Seq. ID No. 5029, Seq. ID No. 5031, Seq. ID No. 5033, Seq. ID No. 5035, Seq. ID No. 5041, Seq. ID No. 5043, Seq. ID No. 5045, Seq. ID No. 5047, Seq. ID No. 5057, Seq. ID No. 5059, Seq. ID No. 5063, Seq. ID No. 5065, Seq. ID No. 5067, Seq. ID No. 5069, Seq. ID No. 5075, Seq. ID No. 5077, Seq. ID No. 5079, Seq. ID No. 5081, Seq. ID No. 5083, Seq. ID No. 5085, Seq. ID No. 5087, Seq. ID No. 5091, Seq. ID No. 5099, Seq. ID No. 5101, Seq. ID No. 5103, Seq. ID No. 5109, Seq. ID No. 5111, Seq. ID No. 5113, Seq. ID No. 5115, Seq. ID No. 5117, Seq. ID No. 5121, Seq. ID No. 5125, Seq. ID No. 5127, Seq. ID No. 5133, Seq. ID No. 5139, Seq. ID No. 5141, Seq. ID No. 5145, Seq. ID No. 5147, Seq. ID No. 5149, Seq. ID No. 5151, Seq. ID No. 5155, Seq. ID No. 5157, Seq. ID No. 5159, Seq. ID No. 5161, Seq. ID No. 5165, Seq. ID No. 5167, Seq. ID No. 5169, Seq. ID No. 5177, Seq. ID No. 5181, Seq. ID No. 5183, Seq. ID No. 5185, Seq. ID No. 5187, Seq. ID No. 5189, Seq. ID No. 5191, Seq. ID No. 5193, Seq. ID No. 5195, Seq. ID No. 5197, Seq. ID No. 5199, Seq. ID No. 5201, Seq. ID No. 5203, Seq. ID No. 5205, Seq. ID No. 5209, Seq. ID No. 5211, Seq. ID No. 5215, Seq. ID No. 5219, Seq. ID No. 5221, Seq. ID No. 5229, Seq. ID No. 5231, Seq. ID No. 5239, Seq. ID No. 5241, Seq. ID No. 5243, Seq. ID No. 5245, Seq. ID No. 5251, Seq. ID No. 5253, Seq. ID No. 5255, Seq. ID No. 5261, Seq. ID No. 5263, Seq. ID No. 5265, Seq. ID No. 5267, Seq. ID No. 5277, Seq. ID No. 5279, Seq. ID No. 5281, Seq. ID No. 5285, Seq. ID No. 5287, Seq. ID No. 5291, Seq. ID No. 5295, Seq. ID No. 5297, Seq. ID No. 5299, Seq. ID No. 5301, Seq. ID No. 5303, Seq. ID No. 5305, Seq. ID No. 5307, Seq. ID No. 5309, Seq. ID No. 5311, Seq. ID No. 5313, Seq. ID No. 5317, Seq. ID No. 5319, Seq. ID No. 5321, Seq. ID No. 5323, Seq. ID No. 5327, Seq. ID No. 5329, Seq. ID No. 5331, Seq. ID No. 5333, Seq. ID No. 5335, Seq. ID No. 5337, Seq. ID No. 5343, Seq. ID No. 5345, Seq. ID No. 5349, Seq. ID No. 5351, Seq. ID No. 5357, Seq. ID No. 5359, Seq. ID No. 5365, Seq. ID No. 5367, Seq. ID No. 5373, Seq. ID No. 5377, Seq. ID No. 5379, Seq. ID No. 5381, Seq. ID No. 5383, Seq. ID No. 5387, Seq. ID No. 5389, Seq. ID No. 5391, Seq. ID No. 5393, Seq. ID No. 5397, Seq. ID No. 5399, Seq. ID No. 5401, Seq. ID No. 5403, Seq. ID No. 5405, Seq. ID No. 5407, Seq. ID No. 5409, Seq. ID No. 5411, Seq. ID No. 5415, Seq. ID No. 5417, Seq. ID No. 5419, Seq. ID No. 5421, Seq. ID No. 5423, Seq. ID No. 5425, Seq. ID No. 5427, Seq. ID No. 5431, Seq. ID No. 5433, Seq. ID No. 5435, Seq. ID No. 5437, Seq. ID No. 5439, Seq. ID No. 5441, Seq. ID No. 5443, Seq. ID No. 5447, Seq. ID No. 5449, Seq. ID No. 5451, Seq. ID No. 5453, Seq. ID No. 5455, Seq. ID No. 5457, Seq. ID No. 5467, Seq. ID No. 5471, Seq. ID No. 5473, Seq. ID No. 5477, Seq. ID No. 5479, Seq. ID No. 5481, Seq. ID No. 5491, Seq. ID No. 5493, Seq. ID No. 5495, Seq. ID No. 5499, Seq. ID No. 5503, Seq. ID No. 5509, Seq. ID No. 5513, Seq. ID No. 5515, Seq. ID No. 5517, Seq. ID No. 5519, Seq. ID No. 5521, Seq. ID No. 5527, Seq. ID No. 5529, Seq. ID No. 5531, Seq. ID No. 5533, Seq. ID No. 5537, Seq. ID No. 5539, Seq. ID No. 5541, Seq. ID No. 5543, Seq. ID No. 5545, Seq. ID No. 5547, Seq. ID No. 5549, Seq. ID No. 5551, Seq. ID No. 5553, Seq. ID No. 5557, Seq. ID No. 5559, Seq. ID No. 5563, Seq. ID No. 5567, Seq. ID No. 5569, Seq. ID No. 5575, Seq. ID No. 5577, Seq. ID No. 5581, Seq. ID No. 5583, Seq. ID No. 5585, Seq. ID No. 5589, Seq. ID No. 5591, Seq. ID No. 5593, Seq. ID No. 5595, Seq. ID No. 5599, Seq. ID No. 5601, Seq. ID No. 5607, Seq. ID No. 5613, Seq. ID No. 5615, Seq. ID No. 5617, Seq. ID No. 5619, Seq. ID No. 5621, Seq. ID No. 5625, Seq. ID No. 5627, Seq. ID No. 5629, Seq. ID No. 5631, Seq. ID No. 5637, Seq. ID No. 5639, Seq. ID No. 5641, Seq. ID No. 5645, Seq. ID No. 5647, Seq. ID No. 5649, Seq. ID No. 5651, Seq. ID No. 5655, Seq. ID No. 5657, Seq. ID No. 5661, Seq. ID No. 5665, Seq. ID No. 5667, Seq. ID No. 5669, Seq. ID No. 5677, Seq. ID No. 5679, Seq. ID No. 5683, Seq. ID No. 5685, Seq. ID No. 5687, Seq. ID No. 5689, Seq. ID No. 5691, Seq. ID No. 5693, Seq. ID No. 5697, Seq. ID No. 5699, Seq. ID No. 5701, Seq. ID No. 5703, Seq. ID No. 5705, Seq. ID No. 5707, Seq. ID No. 5711, Seq. ID No. 5713, Seq. ID No. 5715, Seq. ID No. 5717, Seq. ID No. 5719, Seq. ID No. 5721, Seq. ID No. 5723, Seq. ID No. 5725, Seq. ID No. 5727, Seq. ID No. 5735, Seq. ID No. 5739, Seq. ID No. 5741, Seq. ID No. 5743, Seq. ID No. 5745, Seq. ID No. 5747, Seq. ID No. 5749, Seq. ID No. 5751, Seq. ID No. 5753, Seq. ID No. 5755, Seq. ID No. 5757, Seq. ID No. 5759, Seq. ID No. 5761, Seq. ID No. 5769, Seq. ID No. 5771, Seq. ID No. 5777, Seq. ID No. 5779, Seq. ID No. 5783, Seq. ID No. 5787, Seq. ID No. 5791, Seq. ID No. 5795, Seq. ID No. 5799, Seq. ID No. 5801, Seq. ID No. 5807, Seq. ID No. 5809, Seq. ID No. 5813, Seq. ID No. 5815, Seq. ID No. 5819, Seq. ID No. 5821, Seq. ID No. 5823, Seq. ID No. 5825, Seq. ID No. 5827, Seq. ID No. 5829, Seq. ID No. 5831, Seq. ID No. 5833, Seq. ID No. 5835, Seq. ID No. 5837, Seq. ID No. 5839, Seq. ID No. 5841, Seq. ID No. 5845, Seq. ID No. 5847, Seq. ID No. 5849, Seq. ID No. 5851, Seq. ID No. 5855, Seq. ID No. 5857, Seq. ID No. 5859, Seq. ID No. 5861, Seq. ID No. 5863, Seq. ID No. 5865, Seq. ID No. 5869, Seq. ID No. 5871, Seq. ID No. 5873, Seq. ID No. 5875, Seq. ID No. 5879, Seq. ID No. 5881, Seq. ID No. 5883, Seq. ID No. 5889, Seq. ID No. 5895, Seq. ID No. 5897, Seq. ID No. 5901, Seq. ID No. 5903, Seq. ID No. 5911, Seq. ID No. 5913, Seq. ID No. 5915, Seq. ID No. 5917, Seq. ID No. 5919, Seq. ID No. 5927, Seq. ID No. 5929, Seq. ID No. 5933, Seq. ID No. 5935, Seq. ID No. 5937, Seq. ID No. 5941, Seq. ID No. 5945, Seq. ID No. 5947, Seq. ID No. 5951, Seq. ID No. 5959, Seq. ID No. 5961, Seq. ID No. 5963, Seq. ID No. 5965, Seq. ID No. 5967, Seq. ID No. 5969, Seq. ID No. 5971, Seq. ID No. 5973, Seq. ID No. 5977, Seq. ID No. 5979, Seq. ID No. 5981, Seq. ID No. 5985, Seq. ID No. 5987, Seq. ID No. 5989, Seq. ID No. 5991, Seq. ID No. 5993, Seq. ID No. 5995, Seq. ID No. 5999, Seq. ID No. 6001, Seq. ID No. 6003, Seq. ID No. 6009, Seq. ID No. 6011, Seq. ID No. 6013, Seq. ID No. 6015, Seq. ID No. 6017, Seq. ID No. 6025, Seq. ID No. 6027, Seq. ID No. 6029, Seq. ID No. 6031, Seq. ID No. 6033, Seq. ID No. 6035, Seq. ID No. 6039, Seq. ID No. 6041, Seq. ID No. 6043, Seq. ID No. 6045, Seq. ID No. 6049, Seq. ID No. 6051, Seq. ID No. 6053, Seq. ID No. 6055, Seq. ID No. 6057, Seq. ID No. 6061, Seq. ID No. 6063, Seq. ID No. 6065, Seq. ID No. 6069, Seq. ID No. 6071, Seq. ID No. 6073, Seq. ID No. 6077, Seq. ID No. 6079, Seq. ID No. 6085, Seq. ID No. 6087, Seq. ID No. 6089, Seq. ID No. 6093, Seq. ID No. 6095, Seq. ID No. 6101, Seq. ID No. 6103, Seq. ID No. 6109, Seq. ID No. 6111, Seq. ID No. 6117, Seq. ID No. 6121, Seq. ID No. 6123, Seq. ID No. 6129, Seq. ID No. 6131, Seq. ID No. 6133, Seq. ID No. 6135, Seq. ID No. 6137, Seq. ID No. 6139, Seq. ID No. 6141, Seq. ID No. 6145, Seq. ID No. 6151, Seq. ID No. 6155, Seq. ID No. 6157, Seq. ID No. 6159, Seq. ID No. 6165, Seq. ID No. 6167, Seq. ID No. 6169, Seq. ID No. 6171, Seq. ID No. 6175, Seq. ID No. 6177, Seq. ID No. 6181, Seq. ID No. 6187, Seq. ID No. 6193, Seq. ID No. 6195, Seq. ID No. 6197, Seq. ID No. 6199, Seq. ID No. 6201, Seq. ID No. 6209, Seq. ID No. 6213, Seq. ID No. 6215, Seq. ID No. 6217, Seq. ID No. 6219, Seq. ID No. 6231, Seq. ID No. 6233, Seq. ID No. 6235, Seq. ID No. 6239, Seq. ID No. 6241, Seq. ID No. 6243, Seq. ID No. 6249, Seq. ID No. 6257, Seq. ID No. 6259, Seq. ID No. 6261, Seq. ID No. 6263, Seq. ID No. 6267, Seq. ID No. 6269, Seq. ID No. 6271, Seq. ID No. 6275, Seq. ID No. 6277, Seq. ID No. 6279, Seq. ID No. 6281, Seq. ID No. 6283, Seq. ID No. 6285, Seq. ID No. 6287, Seq. ID No. 6293, Seq. ID No. 6295, Seq. ID No. 6297, Seq. ID No. 6299, Seq. ID No. 6305, Seq. ID No. 6309, Seq. ID No. 6311, Seq. ID No. 6315, Seq. ID No. 6317, Seq. ID No. 6319, Seq. ID No. 6321, Seq. ID No. 6323, Seq. ID No. 6325, Seq. ID No. 6327, Seq. ID No. 6329, Seq. ID No. 6331, Seq. ID No. 6335, Seq. ID No. 6337, Seq. ID No. 6341, Seq. ID No. 6345, Seq. ID No. 6349, Seq. ID No. 6351, Seq. ID No. 6353, Seq. ID No. 6359, Seq. ID No. 6361, Seq. ID No. 6367, Seq. ID No. 6369, Seq. ID No. 6371, Seq. ID No. 6373, Seq. ID No. 6375, Seq. ID No. 6377, Seq. ID No. 6379, Seq. ID No. 6381, Seq. ID No. 6385, Seq. ID No. 6389, Seq. ID No. 6393, Seq. ID No. 6395, Seq. ID No. 6399, Seq. ID No. 6409, Seq. ID No. 6411, Seq. ID No. 6413, Seq. ID No. 6417, Seq. ID No. 6421, Seq. ID No. 6423, Seq. ID No. 6425, Seq. ID No. 6427, Seq. ID No. 6429, Seq. ID No. 6431, Seq. ID No. 6441, Seq. ID No. 6443, Seq. ID No. 6445, Seq. ID No. 6451, Seq. ID No. 6453, Seq. ID No. 6459, Seq. ID No. 6461, Seq. ID No. 6463, Seq. ID No. 6465, Seq. ID No. 6467, Seq. ID No. 6469, Seq. ID No. 6471, Seq. ID No. 6473, Seq. ID No. 6479, Seq. ID No. 6483, Seq. ID No. 6493, Seq. ID No. 6495, Seq. ID No. 6497, Seq. ID No. 6501, Seq. ID No. 6503, Seq. ID No. 6505, Seq. ID No. 6507, Seq. ID No. 6511, Seq. ID No. 6517, Seq. ID No. 6523, Seq. ID No. 6525, Seq. ID No. 6535, Seq. ID No. 6537, Seq. ID No. 6539, Seq. ID No. 6541, Seq. ID No. 6543, Seq. ID No. 6545, Seq. ID No. 6547, Seq. ID No. 6549, Seq. ID No. 6551, Seq. ID No. 6553, Seq. ID No. 6555, Seq. ID No. 6557, Seq. ID No. 6559, Seq. ID No. 6563, Seq. ID No. 6573, Seq. ID No. 6575, Seq. ID No. 6577, Seq. ID No. 6579, Seq. ID No. 6583, Seq. ID No. 6585, Seq. ID No. 6587, Seq. ID No. 6591, Seq. ID No. 6597, Seq. ID No. 6601, Seq. ID No. 6603, Seq. ID No. 6605, Seq. ID No. 6609, Seq. ID No. 6611, Seq. ID No. 6613, Seq. ID No. 6615, Seq. ID No. 6617, Seq. ID No. 6619, Seq. ID No. 6621, Seq. ID No. 6623, Seq. ID No. 6627, Seq. ID No. 6629, Seq. ID No. 6631, Seq. ID No. 6633, Seq. ID No. 6637, Seq. ID No. 6639, Seq. ID No. 6643, Seq. ID No. 6645, and Seq. ID No. 6649.

In another preferred embodiment, the invention comprises an isolated polynucleotide of the Alloiococcus otitidis genomic sequence encoding a polypeptide, which has been by identified by the Applicants' assignee's ORF finder program that searches for a transmembrane domain between two Stop codons and a Start codon immediately upstream of this transmembrane region, and the polynucleotide is selected from Seq. ID No. 7, Seq. ID No. 9, Seq. ID No. 33, Seq. ID No. 47, Seq. ID No. 67, Seq. ID No. 85, Seq. ID No. 87, Seq. ID No. 123, Seq. ID No. 133, Seq. ID No. 141, Seq. ID No. 159, Seq. ID No. 163, Seq. ID No. 191, Seq. ID No. 209, Seq. ID No. 211, Seq. ID No. 215, Seq. ID No. 217, Seq. ID No. 225, Seq. ID No. 235, Seq. ID No. 237, Seq. ID No. 249, Seq. ID No. 251, Seq. ID No. 255, Seq. ID No. 271, Seq. ID No. 277, Seq. ID No. 285, Seq. ID No. 287, Seq. ID No. 289, Seq. ID No. 327, Seq. ID No. 329, Seq. ID No. 373, Seq. ID No. 375, Seq. ID No. 419, Seq. ID No. 487, Seq. ID No. 497, Seq. ID No. 499, Seq. ID No. 503, Seq. ID No. 505, Seq. ID No. 509, Seq. ID No. 511, Seq. ID No. 515, Seq. ID No. 525, Seq. ID No. 527, Seq. ID No. 545, Seq. ID No. 567, Seq. ID No. 569, Seq. ID No. 575, Seq. ID No. 577, Seq. ID No. 579, Seq. ID No. 593, Seq. ID No. 617, Seq. ID No. 637, Seq. ID No. 639, Seq. ID No. 641, Seq. ID No. 645, Seq. ID No. 653, Seq. ID No. 655, Seq. ID No. 659, Seq. ID No. 669, Seq. ID No. 675, Seq. ID No. 677, Seq. ID No. 679, Seq. ID No. 715, Seq. ID No. 717, Seq. ID No. 719, Seq. ID No. 721, Seq. ID No. 723, Seq. ID No. 747, Seq. ID No. 751, Seq. ID No. 755, Seq. ID No. 757, Seq. ID No. 759, Seq. ID No. 761, Seq. ID No. 763, Seq. ID No. 769, Seq. ID No. 777, Seq. ID No. 779, Seq. ID No. 807, Seq. ID No. 809, Seq. ID No. 819, Seq. ID No. 825, Seq. ID No. 863, Seq. ID No. 893, Seq. ID No. 901, Seq. ID No. 935, Seq. ID No. 943, Seq. ID No. 955, Seq. ID No. 967, Seq. ID No. 979, Seq. ID No. 1005, Seq. ID No. 1009, Seq. ID No. 1011, Seq. ID No. 1013, Seq. ID No. 1015, Seq. ID No. 1019, Seq. ID No. 1029, Seq. ID No. 1031, Seq. ID No. 1033, Seq. ID No. 1037, Seq. ID No. 1039, Seq. ID No. 1045, Seq. ID No. 1055, Seq. ID No. 1075, Seq. ID No. 1105, Seq. ID No. 1131, Seq. ID No. 1135, Seq. ID No. 1137, Seq. ID No. 1165, Seq. ID No. 1167, Seq. ID No. 1169, Seq. ID No. 1183, Seq. ID No. 1193, Seq. ID No. 1197, Seq. ID No. 1213, Seq. ID No. 1223, Seq. ID No. 1227, Seq. ID No. 1229, Seq. ID No. 1237, Seq. ID No. 1243, Seq. ID No. 1265, Seq. ID No. 1267, Seq. ID No. 1269, Seq. ID No. 1271, Seq. ID No. 1283, Seq. ID No. 1287, Seq. ID No. 1291, Seq. ID No. 1297, Seq. ID No. 1305, Seq. ID No. 1315, Seq. ID No. 1325, Seq. ID No. 1327, Seq. ID No. 1329, Seq. ID No. 1331, Seq. ID No. 1341, Seq. ID No. 1353, Seq. ID No. 1367, Seq. ID No. 1401, Seq. ID No. 1429, Seq. ID No. 1435, Seq. ID No. 1445, Seq. ID No. 1481, Seq. ID No. 1513, Seq. ID No. 1515, Seq. ID No. 1517, Seq. ID No. 1529, Seq. ID No. 1533, Seq. ID No. 1535, Seq. ID No. 1545, Seq. ID No. 1547, Seq. ID No. 1551, Seq. ID No. 1559, Seq. ID No. 1563, Seq. ID No. 1571, Seq. ID No. 1579, Seq. ID No. 1583, Seq. ID No. 1585, Seq. ID No. 1621, Seq. ID No. 1633, Seq. ID No. 1643, Seq. ID No. 1645, Seq. ID No. 1647, Seq. ID No. 1649, Seq. ID No. 1653, Seq. ID No. 1671, Seq. ID No. 1673, Seq. ID No. 1677, Seq. ID No. 1681, Seq. ID No. 1707, Seq. ID No. 1731, Seq. ID No. 1733, Seq. ID No. 1737, Seq. ID No. 1753, Seq. ID No. 1757, Seq. ID No. 1765, Seq. ID No. 1767, Seq. ID No. 1787, Seq. ID No. 1791, Seq. ID No. 1797, Seq. ID No. 1805, Seq. ID No. 1819, Seq. ID No. 1821, Seq. ID No. 1823, Seq. ID No. 1839, Seq. ID No. 1869, Seq. ID No. 1875, Seq. ID No. 1891, Seq. ID No. 1893, Seq. ID No. 1895, Seq. ID No. 1923, Seq. ID No. 1927, Seq. ID No. 1969, Seq. ID No. 1981, Seq. ID No. 1991, Seq. ID No. 1993, Seq. ID No. 2013, Seq. ID No. 2017, Seq. ID No. 2027, Seq. ID No. 2033, Seq. ID No. 2041, Seq. ID No. 2049, Seq. ID No. 2053, Seq. ID No. 2087, Seq. ID No. 2089, Seq. ID No. 2105, Seq. ID No. 2115, Seq. ID No. 2121, Seq. ID No. 2127, Seq. ID No. 2151, Seq. ID No. 2159, Seq. ID No. 2171, Seq. ID No. 2173, Seq. ID No. 2177, Seq. ID No. 2187, Seq. ID No. 2237, Seq. ID No. 2239, Seq. ID No. 2245, Seq. ID No. 2249, Seq. ID No. 2271, Seq. ID No. 2283, Seq. ID No. 2289, Seq. ID No. 2291, Seq. ID No. 2293, Seq. ID No. 2295, Seq. ID No. 2303, Seq. ID No. 2305, Seq. ID No. 2307, Seq. ID No. 2317, Seq. ID No. 2319, Seq. ID No. 2321, Seq. ID No. 2323, Seq. ID No. 2325, Seq. ID No. 2327, Seq. ID No. 2333, Seq. ID No. 2343, Seq. ID No. 2347, Seq. ID No. 2349, Seq. ID No. 2351, Seq. ID No. 2353, Seq. ID No. 2355, Seq. ID No. 2357, Seq. ID No. 2365, Seq. ID No. 2389, Seq. ID No. 2391, Seq. ID No. 2407, Seq. ID No. 2419, Seq. ID No. 2421, Seq. ID No. 2427, Seq. ID No. 2429, Seq. ID No. 2433, Seq. ID No. 2449, Seq. ID No. 2459, Seq. ID No. 2489, Seq. ID No. 2497, Seq. ID No. 2503, Seq. ID No. 2507, Seq. ID No. 2515, Seq. ID No. 2523, Seq. ID No. 2525, Seq. ID No. 2531, Seq. ID No. 2535, Seq. ID No. 2537, Seq. ID No. 2541, Seq. ID No. 2543, Seq. ID No. 2545, Seq. ID No. 2555, Seq. ID No. 2557, Seq. ID No. 2569, Seq. ID No. 2571, Seq. ID No. 2583, Seq. ID No. 2591, Seq. ID No. 2597, Seq. ID No. 2599, Seq. ID No. 2601, Seq. ID No. 2607, Seq. ID No. 2611, Seq. ID No. 2621, Seq. ID No. 2629, Seq. ID No. 2651, Seq. ID No. 2679, Seq. ID No. 2693, Seq. ID No. 2701, Seq. ID No. 2703, Seq. ID No. 2705, Seq. ID No. 2707, Seq. ID No. 2731, Seq. ID No. 2733, Seq. ID No. 2783, Seq. ID No. 2785, Seq. ID No. 2813, Seq. ID No. 2815, Seq. ID No. 2823, Seq. ID No. 2835, Seq. ID No. 2837, Seq. ID No. 2841, Seq. ID No. 2853, Seq. ID No. 2873, Seq. ID No. 2875, Seq. ID No. 2881, Seq. ID No. 2905, Seq. ID No. 2935, Seq. ID No. 2937, Seq. ID No. 2939, Seq. ID No. 2941, Seq. ID No. 2947, Seq. ID No. 2949, Seq. ID No. 2969, Seq. ID No. 2973, Seq. ID No. 2977, Seq. ID No. 2981, Seq. ID No. 2987, Seq. ID No. 2991, Seq. ID No. 2995, Seq. ID No. 2997, Seq. ID No. 3005, Seq. ID No. 3007, Seq. ID No. 3013, Seq. ID No. 3023, Seq. ID No. 3025, Seq. ID No. 3027, Seq. ID No. 3029, Seq. ID No. 3031, Seq. ID No. 3043, Seq. ID No. 3055, Seq. ID No. 3063, Seq. ID No. 3071, Seq. ID No. 3073, Seq. ID No. 3075, Seq. ID No. 3077, Seq. ID No. 3123, Seq. ID No. 3145, Seq. ID No. 3155, Seq. ID No. 3157, Seq. ID No. 3163, Seq. ID No. 3183, Seq. ID No. 3193, Seq. ID No. 3201, Seq. ID No. 3213, Seq. ID No. 3215, Seq. ID No. 3217, Seq. ID No. 3219, Seq. ID No. 3241, Seq. ID No. 3243, Seq. ID No. 3245, Seq. ID No. 3249, Seq. ID No. 3257, Seq. ID No. 3261, Seq. ID No. 3269, Seq. ID No. 3275, Seq. ID No. 3297, Seq. ID No. 3317, Seq. ID No. 3323, Seq. ID No. 3337, Seq. ID No. 3339, Seq. ID No. 3349, Seq. ID No. 3351, Seq. ID No. 3353, Seq. ID No. 3357, Seq. ID No. 3359, Seq. ID No. 3377, Seq. ID No. 3389, Seq. ID No. 3391, Seq. ID No. 3395, Seq. ID No. 3405, Seq. ID No. 3421, Seq. ID No. 3425, Seq. ID No. 3449, Seq. ID No. 3451, Seq. ID No. 3453, Seq. ID No. 3455, Seq. ID No. 3457, Seq. ID No. 3463, Seq. ID No. 3481, Seq. ID No. 3491, Seq. ID No. 3493, Seq. ID No. 3503, Seq. ID No. 3505, Seq. ID No. 3527, Seq. ID No. 3593, Seq. ID No. 3595, Seq. ID No. 3621, Seq. ID No. 3625, Seq. ID No. 3635, Seq. ID No. 3679, Seq. ID No. 3723, Seq. ID No. 3727, Seq. ID No. 3745, Seq. ID No. 3753, Seq. ID No. 3755, Seq. ID No. 3757, Seq. ID No. 3761, Seq. ID No. 3763, Seq. ID No. 3765, Seq. ID No. 3783, Seq. ID No. 3793, Seq. ID No. 3799, Seq. ID No. 3813, Seq. ID No. 3837, Seq. ID No. 3839, Seq. ID No. 3849, Seq. ID No. 3851, Seq. ID No. 3853, Seq. ID No. 3859, Seq. ID No. 3861, Seq. ID No. 3863, Seq. ID No. 3865, Seq. ID No. 3879, Seq. ID No. 3889, Seq. ID No. 3891, Seq. ID No. 3903, Seq. ID No. 3925, Seq. ID No. 3963, Seq. ID No. 3973, Seq. ID No. 3975, Seq. ID No. 4005, Seq. ID No. 4025, Seq. ID No. 4027, Seq. ID No. 4039, Seq. ID No. 4041, Seq. ID No. 4043, Seq. ID No. 4045, Seq. ID No. 4047, Seq. ID No. 4061, Seq. ID No. 4071, Seq. ID No. 4073, Seq. ID No. 4079, Seq. ID No. 4081, Seq. ID No. 4083, Seq. ID No. 4101, Seq. ID No. 4103, Seq. ID No. 4111, Seq. ID No. 4133, Seq. ID No. 4137, Seq. ID No. 4139, Seq. ID No. 4145, Seq. ID No. 4151, Seq. ID No. 4221, Seq. ID No. 4223, Seq. ID No. 4225, Seq. ID No. 4227, Seq. ID No. 4231, Seq. ID No. 4241, Seq. ID No. 4247, Seq. ID No. 4253, Seq. ID No. 4257, Seq. ID No. 4261, Seq. ID No. 4307, Seq. ID No. 4311, Seq. ID No. 4313, Seq. ID No. 4343, Seq. ID No. 4379, Seq. ID No. 4385, Seq. ID No. 4389, Seq. ID No. 4439, Seq. ID No. 4469, Seq. ID No. 4473, Seq. ID No. 4481, Seq. ID No. 4483, Seq. ID No. 4485, Seq. ID No. 4497, Seq. ID No. 4515, Seq. ID No. 4533, Seq. ID No. 4587, Seq. ID No. 4639, Seq. ID No. 4643, Seq. ID No. 4681, Seq. ID No. 4705, Seq. ID No. 4707, Seq. ID No. 4709, Seq. ID No. 4711, Seq. ID No. 4715, Seq. ID No. 4717, Seq. ID No. 4737, Seq. ID No. 4759, Seq. ID No. 4813, Seq. ID No. 4827, Seq. ID No. 4869, Seq. ID No. 4871, Seq. ID No. 4873, Seq. ID No. 4875, Seq. ID No. 4919, Seq. ID No. 4931, Seq. ID No. 4949, Seq. ID No. 4951, Seq. ID No. 4987, Seq. ID No. 5031, Seq. ID No. 5061, Seq. ID No. 5085, Seq. ID No. 5103, Seq. ID No. 5123, Seq. ID No. 5129, Seq. ID No. 5131, Seq. ID No. 5135, Seq. ID No. 5137, Seq. ID No. 5141, Seq. ID No. 5165, Seq. ID No. 5175, Seq. ID No. 5199, Seq. ID No. 5205, Seq. ID No. 5207, Seq. ID No. 5213, Seq. ID No. 5219, Seq. ID No. 5223, Seq. ID No. 5267, Seq. ID No. 5281, Seq. ID No. 5385, Seq. ID No. 5387, Seq. ID No. 5413, Seq. ID No. 5415, Seq. ID No. 5433, Seq. ID No. 5453, Seq. ID No. 5459, Seq. ID No. 5461, Seq. ID No. 5463, Seq. ID No. 5465, Seq. ID No. 5491, Seq. ID No. 5497, Seq. ID No. 5509, Seq. ID No. 5513, Seq. ID No. 5525, Seq. ID No. 5531, Seq. ID No. 5569, Seq. ID No. 5587, Seq. ID No. 5595, Seq. ID No. 5597, Seq. ID No. 5609, Seq. ID No. 5611, Seq. ID No. 5621, Seq. ID No. 5623, Seq. ID No. 5633, Seq. ID No. 5637, Seq. ID No. 5643, Seq. ID No. 5649, Seq. ID No. 5659, Seq. ID No. 5683, Seq. ID No. 5685, Seq. ID No. 5699, Seq. ID No. 5705, Seq. ID No. 5709, Seq. ID No. 5729, Seq. ID No. 5733, Seq. ID No. 5763, Seq. ID No. 5765, Seq. ID No. 5767, Seq. ID No. 5771, Seq. ID No. 5783, Seq. ID No. 5791, Seq. ID No. 5797, Seq. ID No. 5885, Seq. ID No. 5889, Seq. ID No. 5893, Seq. ID No. 5895, Seq. ID No. 5899, Seq. ID No. 5901, Seq. ID No. 5905, Seq. ID No. 5909, Seq. ID No. 5921, Seq. ID No. 5923, Seq. ID No. 5927, Seq. ID No. 5939, Seq. ID No. 6027, Seq. ID No. 6043, Seq. ID No. 6047, Seq. ID No. 6059, Seq. ID No. 6067, Seq. ID No. 6089, Seq. ID No. 6091, Seq. ID No. 6107, Seq. ID No. 6111, Seq. ID No. 6117, Seq. ID No. 6161, Seq. ID No. 6163, Seq. ID No. 6165, Seq. ID No. 6169, Seq. ID No. 6183, Seq. ID No. 6185, Seq. ID No. 6189, Seq. ID No. 6191, Seq. ID No. 6197, Seq. ID No. 6203, Seq. ID No. 6205, Seq. ID No. 6209, Seq. ID No. 6221, Seq. ID No. 6223, Seq. ID No. 6225, Seq. ID No. 6227, Seq. ID No. 6229, Seq. ID No. 6235, Seq. ID No. 6237, Seq. ID No. 6243, Seq. ID No. 6245, Seq. ID No. 6249, Seq. ID No. 6253, Seq. ID No. 6263, Seq. ID No. 6265, Seq. ID No. 6273, Seq. ID No. 6275, Seq. ID No. 6285, Seq. ID No. 6289, Seq. ID No. 6301, Seq. ID No. 6303, Seq. ID No. 6363, Seq. ID No. 6377, Seq. ID No. 6381, Seq. ID No. 6383, Seq. ID No. 6415, Seq. ID No. 6427, Seq. ID No. 6431, Seq. ID No. 6473, Seq. ID No. 6497, Seq. ID No. 6571, Seq. ID No. 6581, Seq. ID No. 6593, Seq. ID No. 6599, Seq. ID No. 6631, and Seq. ID No. 6639.

In one preferred embodiment,the invention comprises an isolated polynucleotide of an Alloiococcus otitidis genomic sequence, wherein the polynucleotide comprises a nucleotide sequence having at least about 70% identity to a nucleotide sequence contained within SEQ ID NO: 6651, a complement thereof, a degenerate variant thereof, and a fragment thereof.

In other preferred embodiments, the invention comprises an isolated polypeptide encoded by a polynucleotide and having at least about 70% identity to a nucleotide sequence chosen from one of the odd numbered sequence listings set out in SEQ ID NO: 1 through SEQ ID NO: 6649.

In certain aspects, the invention relates to Alloiococcus otitidis polypeptides. More particularly, the invention relates to Alloiococcus otitidis polypeptides, more preferably antigenic polypeptides, encoded by Alloiococcus otitidis polynucleotide open reading frames. Thus, in certain embodiments, an isolated polypeptide is encoded by a polynucleotide comprising a nucleotide sequence having at least about 70% identity to a nucleotide sequence chosen from one of odd numbered sequences set out in SEQ ID NO: 1 through SEQ ID NO: 6649, a complement thereof, a degenerate variant thereof, or a fragment thereof. In a preferred embodiment, the isolated polypeptide encoded by one of the above polynucleotides comprises an amino acid sequence having at least about 70% identity to an amino acid sequence chosen from one of the even numbered sequences set out in SEQ ID NO: 2 through SEQ ID NO: 6650, a biological equivalent thereof, or a fragment thereof. In other embodiments, the polypeptide is a fusion polypeptide. In a preferred embodiment, the polypeptide immunoreacts with seropositive serum of an individual previously infected with Alloiococcus otitidis, a biological equivalent thereof, or a fragment thereof.

In other preferred embodiments, the isolated polypeptide encoded is selected from (a) an Alloiococcus otitidis polypeptide predicted by SignalP algorithm analysis as having met all four criteria set out in the SignalP algorithm for a polypeptide having a signal peptide and a subset of these are predicted by HMM SignalP algorithm analysis as having a signal peptide; (b) an Alloiococcus otitidis polypeptide predicted by SignalP algorithm analysis as having met three of the four criteria set out in the SignalP algorithm for a polypeptide having a signal peptide and a subset of these predicted by HMM SignalP algorithm analysis as having a signal peptide; (c) an Alloiococcus otitidis polypeptide predicted by HMM SignalP algorithm analysis as having a signal peptide; (d) an Alloiococcus otitidis polypeptide predicted by HMM SignalP algorithm analysis as being a non-secretory protein; (e) an Alloiococcus otitidis polypeptide identified by Pfam analysis; (f) an Alloiococcus otitidis polypeptide identified by BlastP analysis; (g) an Alloiococcus otitidis polypeptide identified using proteomics methodology as sharing homology with surface exposed proteins of Streptococcus pneumoniae; (h) an Alloiococcus otitidis polypeptide having a LPXTG motif, wherein the polypeptide is covalently attached to the peptidoglycan layer; (i) an Alloiococcus otitidis lipoprotein; (j) an Alloiococcus otitidis polypeptide non-covalently associated with the peptidoglycan layer; (k) an Alloiococcus otitidis polypeptide having an RGDX motif wherein X is not a proline residue; (l) an Alloiococcus otitidis polypeptide predicted by BlastP as being involved in capsule biosynthesis and transport; (m) an Alloiococcus otitidis polypeptide identified by BlastP as being localized within the capsule loci of the Alloiococcus otitidis genome; (n) an Alloiococcus otitidis polypeptide predicted by BlastP as being associated with sporulation; (o) an Alloiococcus otitidis polypeptide encoded by unique ORFs identified by BlastP analysis as having a BlastP ‘E Value’ of >e⁻¹⁰; (p) an Alloiococcus otitidis polypeptide identified by Glimmer™ ORF finder program; (q) an Alloiococcus otitidis polypeptide identified by GeneMark™ ORF finder program; (r) an Alloiococcus otitidis polypeptide identified by Applicants' assignee's ORF finder program that searches for an ATG, GTG or TTG Start codon between a Stop-Stop region; and (s) an Alloiococcus otitidis polypeptide identified by Applicants' assignee's ORF finder program that searches for a transmembrane domain between two Stop codons and a Start codon immediately upstream of this transmembrane region.

In a preferred embodiment, the polypeptide is selected from the Alloiococcus otitidis polypeptides predicted by SignalP algorithm analysis as having met all four criteria set out in the SignalP algorithm for a polypeptide having a signal peptide and is selected from one of Seq. ID No. 90, Seq. ID No. 128, Seq. ID No. 132, Seq. ID No. 134, Seq. ID No. 162, Seq. ID No. 164, Seq. ID No. 214, Seq. ID No. 216, Seq. ID No. 226, Seq. ID No. 250, Seq. ID No. 252, Seq. ID No. 288, Seq. ID No. 290, Seq. ID No. 370, Seq. ID No. 372, Seq. ID No. 376, Seq. ID No. 378, Seq. ID No. 498, Seq. ID No. 504, Seq. ID No. 506, Seq. ID No. 528, Seq. ID No. 550, Seq. ID No. 576, Seq. ID No. 618, Seq. ID No. 658, Seq. ID No. 660, Seq. ID No. 662, Seq. ID No. 676, Seq. ID No. 716, Seq. ID No. 718, Seq. ID No. 746, Seq. ID No. 748, Seq. ID No. 756, Seq. ID No. 758, Seq. ID No. 760, Seq. ID No. 762, Seq. ID No. 774, Seq. ID No. 808, Seq. ID No. 968, Seq. ID No. 974, Seq. ID No. 976, Seq. ID No. 1014, Seq. ID No. 1026, Seq. ID No. 1028, Seq. ID No. 1040, Seq. ID No. 1076, Seq. ID No. 1090, Seq. ID No. 1196, Seq. ID No. 1198, Seq. ID No. 1208, Seq. ID No. 1240, Seq. ID No. 1244, Seq. ID No. 1246, Seq. ID No. 1270, Seq. ID No. 1316, Seq. ID No. 1318, Seq. ID No. 1332, Seq. ID No. 1366, Seq. ID No. 1368, Seq. ID No. 1370, Seq. ID No. 1446, Seq. ID No. 1448, Seq. ID No. 1514, Seq. ID No. 1518, Seq. ID No. 1546, Seq. ID No. 1550, Seq. ID No. 1562, Seq. ID No. 1570, Seq. ID No. 1572, Seq. ID No. 1586, Seq. ID No. 1644, Seq. ID No. 1654, Seq. ID No. 1662, Seq. ID No. 1664, Seq. ID No. 1708, Seq. ID No. 1734, Seq. ID No. 1738, Seq. ID No. 1756, Seq. ID No. 1766, Seq. ID No. 1768, Seq. ID No. 1776, Seq. ID No. 1792, Seq. ID No. 1806, Seq. ID No. 1876, Seq. ID No. 1970, Seq. ID No. 1982, Seq. ID No. 2054, Seq. ID No. 2056, Seq. ID No. 2088, Seq. ID No. 2090, Seq. ID No. 2092, Seq. ID No. 2106, Seq. ID No. 2116, Seq. ID No. 2150, Seq. ID No. 2152, Seq. ID No. 2160, Seq. ID No. 2178, Seq. ID No. 2278, Seq. ID No. 2294, Seq. ID No. 2322, Seq. ID No. 2348, Seq. ID No. 2352, Seq. ID No. 2354, Seq. ID No. 2366, Seq. ID No. 2396, Seq. ID No. 2422, Seq. ID No. 2426, Seq. ID No. 2428, Seq. ID No. 2430, Seq. ID No. 2450, Seq. ID No. 2460, Seq. ID No. 2526, Seq. ID No. 2532, Seq. ID No. 2608, Seq. ID No. 2652, Seq. ID No. 2680, Seq. ID No. 2694, Seq. ID No. 2700, Seq. ID No. 2704, Seq. ID No. 2754, Seq. ID No. 2786, Seq. ID No. 2814, Seq. ID No. 2830, Seq. ID No. 2836, Seq. ID No. 2842, Seq. ID No. 2880, Seq. ID No. 2882, Seq. ID No. 2936, Seq. ID No. 2938, Seq. ID No. 2950, Seq. ID No. 2966, Seq. ID No. 2968, Seq. ID No. 2974, Seq. ID No. 3008, Seq. ID No. 3010, Seq. ID No. 3012, Seq. ID No. 3030, Seq. ID No. 3032, Seq. ID No. 3110, Seq. ID No. 3112, Seq. ID No. 3220, Seq. ID No. 3224, Seq. ID No. 3274, Seq. ID No. 3298, Seq. ID No. 3332, Seq. ID No. 3336, Seq. ID No. 3342, Seq. ID No. 3350, Seq. ID No. 3354, Seq. ID No. 3356, Seq. ID No. 3378, Seq. ID No. 3392, Seq. ID No. 3426, Seq. ID No. 3450, Seq. ID No. 3494, Seq. ID No. 3574, Seq. ID No. 3612, Seq. ID No. 3620, Seq. ID No. 3690, Seq. ID No. 3754, Seq. ID No. 3756, Seq. ID No. 3766, Seq. ID No. 3782, Seq. ID No. 3784, Seq. ID No. 3852, Seq. ID No. 3904, Seq. ID No. 4006, Seq. ID No. 4008, Seq. ID No. 4028, Seq. ID No. 4074, Seq. ID No. 4138, Seq. ID No. 4142, Seq. ID No. 4144, Seq. ID No. 4146, Seq. ID No. 4226, Seq. ID No. 4344, Seq. ID No. 4386, Seq. ID No. 4400, Seq. ID No. 4470, Seq. ID No. 4484, Seq. ID No. 4494, Seq. ID No. 4496, Seq. ID No. 4534, Seq. ID No. 4700, Seq. ID No. 4710, Seq. ID No. 4712, Seq. ID No. 4736, Seq. ID No. 4828, Seq. ID No. 4872, Seq. ID No. 4874, Seq. ID No. 4880, Seq. ID No. 4922, Seq. ID No. 4982, Seq. ID No. 5116, Seq. ID No. 5268, Seq. ID No. 5342, Seq. ID No. 5388, Seq. ID No. 5466, Seq. ID No. 5468, Seq. ID No. 5480, Seq. ID No. 5498, Seq. ID No. 5514, Seq. ID No. 5544, Seq. ID No. 5546, Seq. ID No. 5568, Seq. ID No. 5588, Seq. ID No. 5590, Seq. ID No. 5592, Seq. ID No. 5612, Seq. ID No. 5622, Seq. ID No. 5642, Seq. ID No. 5682, Seq. ID No. 5720, Seq. ID No. 5772, Seq. ID No. 5784, Seq. ID No. 5896, Seq. ID No. 5900, Seq. ID No. 5902, Seq. ID No. 5928, Seq. ID No. 5930, Seq. ID No. 5940, Seq. ID No. 5988, Seq. ID No. 6014, Seq. ID No. 6088, Seq. ID No. 6090, Seq. ID No. 6092, Seq. ID No. 6094, Seq. ID No. 6096, Seq. ID No. 6104, Seq. ID No. 6170, Seq. ID No. 6186, Seq. ID No. 6188, Seq. ID No. 6196, Seq. ID No. 6198, Seq. ID No. 6204, Seq. ID No. 6206, Seq. ID No. 6222, Seq. ID No. 6224, Seq. ID No. 6230, Seq. ID No. 6292, Seq. ID No. 6416, Seq. ID No. 6418, Seq. ID No. 6474, Seq. ID No. 6508, Seq. ID No. 6544, Seq. ID No. 6596, Seq. ID No. 6598, Seq. ID No. 6600, Seq. ID No. 6602, and Seq. ID No. 6604.

In another preferred embodiment, the polypeptide is selected from Alloiococcus otitidis polypeptides predicted by SignalP algorithm analysis as having met three of the four criteria set out in the SignalP algorithm for a polypeptide having a signal peptide and predicted by HMM SignalP algorithm analysis as having a signal peptide and is selected from one of Seq. ID No. 8, Seq. ID No. 12, Seq. ID No. 24, Seq. ID No. 46, Seq. ID No. 48, Seq. ID No. 54, Seq. ID No. 60, Seq. ID No. 66, Seq. ID No. 68, Seq. ID No. 88, Seq. ID No. 92, Seq. ID No. 140, Seq. ID No. 152, Seq. ID No. 160, Seq. ID No. 166, Seq. ID No. 170, Seq. ID No. 186, Seq. ID No. 212, Seq. ID No. 218, Seq. ID No. 236, Seq. ID No. 238, Seq. ID No. 240, Seq. ID No. 272, Seq. ID No. 286, Seq. ID No. 292, Seq. ID No. 328, Seq. ID No. 350, Seq. ID No. 386, Seq. ID No. 388, Seq. ID No. 408, Seq. ID No. 428, Seq. ID No. 432, Seq. ID No. 456, Seq. ID No. 458, Seq. ID No. 496, Seq. ID No. 500, Seq. ID No. 512, Seq. ID No. 514, Seq. ID No. 516, Seq. ID No. 526, Seq. ID No. 546, Seq. ID No. 548, Seq. ID No. 558, Seq. ID No. 570, Seq. ID No. 590, Seq. ID No. 592, Seq. ID No. 594, Seq. ID No. 596, Seq. ID No. 610, Seq. ID No. 638, Seq. ID No. 640, Seq. ID No. 642, Seq. ID No. 654, Seq. ID No. 670, Seq. ID No. 680, Seq. ID No. 720, Seq. ID No. 724, Seq. ID No. 740, Seq. ID No. 750, Seq. ID No. 752, Seq. ID No. 764, Seq. ID No. 766, Seq. ID No. 770, Seq. ID No. 778, Seq. ID No. 780, Seq. ID No. 782, Seq. ID No. 820, Seq. ID No. 896, Seq. ID No. 904, Seq. ID No. 906, Seq. ID No. 910, Seq. ID No. 936, Seq. ID No. 938, Seq. ID No. 952, Seq. ID No. 956, Seq. ID No. 980, Seq. ID No. 982, Seq. ID No. 984, Seq. ID No. 986, Seq. ID No. 1006, Seq. ID No. 1008, Seq. ID No. 1010, Seq. ID No. 1016, Seq. ID No. 1020, Seq. ID No. 1030, Seq. ID No. 1032, Seq. ID No. 1048, Seq. ID No. 1092, Seq. ID No. 1112, Seq. ID No. 1138, Seq. ID No. 1152, Seq. ID No. 1154, Seq. ID No. 1170, Seq. ID No. 1174, Seq. ID No. 1184, Seq. ID No. 1192, Seq. ID No. 1194, Seq. ID No. 1214, Seq. ID No. 1224, Seq. ID No. 1230, Seq. ID No. 1248, Seq. ID No. 1260, Seq. ID No. 1284, Seq. ID No. 1308, Seq. ID No. 1320, Seq. ID No. 1322, Seq. ID No. 1328, Seq. ID No. 1342, Seq. ID No. 1344, Seq. ID No. 1348, Seq. ID No. 1390, Seq. ID No. 1402, Seq. ID No. 1430, Seq. ID No. 1480, Seq. ID No. 1552, Seq. ID No. 1560, Seq. ID No. 1580, Seq. ID No. 1588, Seq. ID No. 1622, Seq. ID No. 1628, Seq. ID No. 1634, Seq. ID No. 1646, Seq. ID No. 1648, Seq. ID No. 1650, Seq. ID No. 1656, Seq. ID No. 1666, Seq. ID No. 1668, Seq. ID No. 1672, Seq. ID No. 1678, Seq. ID No. 1680, Seq. ID No. 1682, Seq. ID No. 1696, Seq. ID No. 1704, Seq. ID No. 1706, Seq. ID No. 1732, Seq. ID No. 1754, Seq. ID No. 1760, Seq. ID No. 1764, Seq. ID No. 1788, Seq. ID No. 1798, Seq. ID No. 1822, Seq. ID No. 1824, Seq. ID No. 1834, Seq. ID No. 1840, Seq. ID No. 1842, Seq. ID No. 1844, Seq. ID No. 1894, Seq. ID No. 1918, Seq. ID No. 1920, Seq. ID No. 1972, Seq. ID No. 1992, Seq. ID No. 2014, Seq. ID No. 2016, Seq. ID No. 2018, Seq. ID No. 2020, Seq. ID No. 2034, Seq. ID No. 2036, Seq. ID No. 2050, Seq. ID No. 2082, Seq. ID No. 2084, Seq. ID No. 2086, Seq. ID No. 2112, Seq. ID No. 2114, Seq. ID No. 2124, Seq. ID No. 2126, Seq. ID No. 2128, Seq. ID No. 2132, Seq. ID No. 2134, Seq. ID No. 2154, Seq. ID No. 2162, Seq. ID No. 2172, Seq. ID No. 2176, Seq. ID No. 2180, Seq. ID No. 2188, Seq. ID No. 2202, Seq. ID No. 2204, Seq. ID No. 2222, Seq. ID No. 2238, Seq. ID No. 2250, Seq. ID No. 2252, Seq. ID No. 2256, Seq. ID No. 2262, Seq. ID No. 2264, Seq. ID No. 2272, Seq. ID No. 2280, Seq. ID No. 2284, Seq. ID No. 2286, Seq. ID No. 2290, Seq. ID No. 2296, Seq. ID No. 2306, Seq. ID No. 2308, Seq. ID No. 2318, Seq. ID No. 2320, Seq. ID No. 2324, Seq. ID No. 2326, Seq. ID No. 2344, Seq. ID No. 2350, Seq. ID No. 2386, Seq. ID No. 2394, Seq. ID No. 2424, Seq. ID No. 2454, Seq. ID No. 2492, Seq. ID No. 2498, Seq. ID No. 2500, Seq. ID No. 2508, Seq. ID No. 2516, Seq. ID No. 2518, Seq. ID No. 2522, Seq. ID No. 2524, Seq. ID No. 2528, Seq. ID No. 2534, Seq. ID No. 2536, Seq. ID No. 2542, Seq. ID No. 2544, Seq. ID No. 2548, Seq. ID No. 2550, Seq. ID No. 2558, Seq. ID No. 2574, Seq. ID No. 2584, Seq. ID No. 2602, Seq. ID No. 2604, Seq. ID No. 2622, Seq. ID No. 2654, Seq. ID No. 2682, Seq. ID No. 2692, Seq. ID No. 2702, Seq. ID No. 2734, Seq. ID No. 2740, Seq. ID No. 2802, Seq. ID No. 2808, Seq. ID No. 2816, Seq. ID No. 2820, Seq. ID No. 2822, Seq. ID No. 2824, Seq. ID No. 2834, Seq. ID No. 2838, Seq. ID No. 2840, Seq. ID No. 2846, Seq. ID No. 2854, Seq. ID No. 2862, Seq. ID No. 2866, Seq. ID No. 2874, Seq. ID No. 2876, Seq. ID No. 2878, Seq. ID No. 2884, Seq. ID No. 2896, Seq. ID No. 2906, Seq. ID No. 2940, Seq. ID No. 2942, Seq. ID No. 2948, Seq. ID No. 2982, Seq. ID No. 2984, Seq. ID No. 2988, Seq. ID No. 2992, Seq. ID No. 2996, Seq. ID No. 3002, Seq. ID No. 3006, Seq. ID No. 3022, Seq. ID No. 3026, Seq. ID No. 3044, Seq. ID No. 3046, Seq. ID No. 3074, Seq. ID No. 3076, Seq. ID No. 3156, Seq. ID No. 3164, Seq. ID No. 3166, Seq. ID No. 3168, Seq. ID No. 3184, Seq. ID No. 3214, Seq. ID No. 3216, Seq. ID No. 3236, Seq. ID No. 3246, Seq. ID No. 3262, Seq. ID No. 3264, Seq. ID No. 3266, Seq. ID No. 3270, Seq. ID No. 3272, Seq. ID No. 3276, Seq. ID No. 3278, Seq. ID No. 3318, Seq. ID No. 3326, Seq. ID No. 3338, Seq. ID No. 3352, Seq. ID No. 3390, Seq. ID No. 3406, Seq. ID No. 3420, Seq. ID No. 3422, Seq. ID No. 3424, Seq. ID No. 3452, Seq. ID No. 3454, Seq. ID No. 3456, Seq. ID No. 3492, Seq. ID No. 3496, Seq. ID No. 3504, Seq. ID No. 3506, Seq. ID No. 3518, Seq. ID No. 3520, Seq. ID No. 3546, Seq. ID No. 3548, Seq. ID No. 3572, Seq. ID No. 3594, Seq. ID No. 3618, Seq. ID No. 3622, Seq. ID No. 3630, Seq. ID No. 3664, Seq. ID No. 3702, Seq. ID No. 3704, Seq. ID No. 3724, Seq. ID No. 3738, Seq. ID No. 3742, Seq. ID No. 3746, Seq. ID No. 3748, Seq. ID No. 3760, Seq. ID No. 3764, Seq. ID No. 3768, Seq. ID No. 3770, Seq. ID No. 3772, Seq. ID No. 3786, Seq. ID No. 3794, Seq. ID No. 3814, Seq. ID No. 3838, Seq. ID No. 3840, Seq. ID No. 3862, Seq. ID No. 3866, Seq. ID No. 3890, Seq. ID No. 3892, Seq. ID No. 3896, Seq. ID No. 3916, Seq. ID No. 3924, Seq. ID No. 3926, Seq. ID No. 3930, Seq. ID No. 3966, Seq. ID No. 3976, Seq. ID No. 3980, Seq. ID No. 3996, Seq. ID No. 3998, Seq. ID No. 4010, Seq. ID No. 4040, Seq. ID No. 4042, Seq. ID No. 4062, Seq. ID No. 4080, Seq. ID No. 4082, Seq. ID No. 4112, Seq. ID No. 4114, Seq. ID No. 4136, Seq. ID No. 4140, Seq. ID No. 4156, Seq. ID No. 4182, Seq. ID No. 4204, Seq. ID No. 4222, Seq. ID No. 4248, Seq. ID No. 4254, Seq. ID No. 4258, Seq. ID No. 4260, Seq. ID No. 4262, Seq. ID No. 4264, Seq. ID No. 4270, Seq. ID No. 4312, Seq. ID No. 4380, Seq. ID No. 4390, Seq. ID No. 4392, Seq. ID No. 4472, Seq. ID No. 4474, Seq. ID No. 4482, Seq. ID No. 4526, Seq. ID No. 4552, Seq. ID No. 4562, Seq. ID No. 4574, Seq. ID No. 4618, Seq. ID No. 4640, Seq. ID No. 4660, Seq. ID No. 4682, Seq. ID No. 4702, Seq. ID No. 4706, Seq. ID No. 4714, Seq. ID No. 4718, Seq. ID No. 4722, Seq. ID No. 4738, Seq. ID No. 4742, Seq. ID No. 4814, Seq. ID No. 4818, Seq. ID No. 4820, Seq. ID No. 4830, Seq. ID No. 4832, Seq. ID No. 4852, Seq. ID No. 4870, Seq. ID No. 4876, Seq. ID No. 4914, Seq. ID No. 4920, Seq. ID No. 4930, Seq. ID No. 4932, Seq. ID No. 4984, Seq. ID No. 4994, Seq. ID No. 5012, Seq. ID No. 5024, Seq. ID No. 5032, Seq. ID No. 5034, Seq. ID No. 5036, Seq. ID No. 5044, Seq. ID No. 5062, Seq. ID No. 5064, Seq. ID No. 5070, Seq. ID No. 5104, Seq. ID No. 5122, Seq. ID No. 5124, Seq. ID No. 5132, Seq. ID No. 5134, Seq. ID No. 5136, Seq. ID No. 5138, Seq. ID No. 5140, Seq. ID No. 5142, Seq. ID No. 5152, Seq. ID No. 5160, Seq. ID No. 5162, Seq. ID No. 5166, Seq. ID No. 5168, Seq. ID No. 5176, Seq. ID No. 5178, Seq. ID No. 5206, Seq. ID No. 5208, Seq. ID No. 5212, Seq. ID No. 5216, Seq. ID No. 5220, Seq. ID No. 5224, Seq. ID No. 5286, Seq. ID No. 5288, Seq. ID No. 5330, Seq. ID No. 5332, Seq. ID No. 5378, Seq. ID No. 5380, Seq. ID No. 5382, Seq. ID No. 5386, Seq. ID No. 5406, Seq. ID No. 5408, Seq. ID No. 5454, Seq. ID No. 5460, Seq. ID No. 5462, Seq. ID No. 5464, Seq. ID No. 5482, Seq. ID No. 5510, Seq. ID No. 5530, Seq. ID No. 5532, Seq. ID No. 5558, Seq. ID No. 5570, Seq. ID No. 5578, Seq. ID No. 5624, Seq. ID No. 5626, Seq. ID No. 5644, Seq. ID No. 5660, Seq. ID No. 5662, Seq. ID No. 5684, Seq. ID No. 5686, Seq. ID No. 5710, Seq. ID No. 5712, Seq. ID No. 5734, Seq. ID No. 5740, Seq. ID No. 5766, Seq. ID No. 5798, Seq. ID No. 5828, Seq. ID No. 5890, Seq. ID No. 5894, Seq. ID No. 5906, Seq. ID No. 5910, Seq. ID No. 5912, Seq. ID No. 5924, Seq. ID No. 5964, Seq. ID No. 5966, Seq. ID No. 6026, Seq. ID No. 6028, Seq. ID No. 6032, Seq. ID No. 6044, Seq. ID No. 6046, Seq. ID No. 6048, Seq. ID No. 6054, Seq. ID No. 6060, Seq. ID No. 6110, Seq. ID No. 6112, Seq. ID No. 6116, Seq. ID No. 6118, Seq. ID No. 6128, Seq. ID No. 6132, Seq. ID No. 6136, Seq. ID No. 6138, Seq. ID No. 6162, Seq. ID No. 6166, Seq. ID No. 6184, Seq. ID No. 6190, Seq. ID No. 6192, Seq. ID No. 6194, Seq. ID No. 6208, Seq. ID No. 6210, Seq. ID No. 6218, Seq. ID No. 6226, Seq. ID No. 6236, Seq. ID No. 6244, Seq. ID No. 6250, Seq. ID No. 6254, Seq. ID No. 6260, Seq. ID No. 6266, Seq. ID No. 6268, Seq. ID No. 6276, Seq. ID No. 6284, Seq. ID No. 6286, Seq. ID No. 6304, Seq. ID No. 6326, Seq. ID No. 6328, Seq. ID No. 6364, Seq. ID No. 6366, Seq. ID No. 6368, Seq. ID No. 6386, Seq. ID No. 6426, Seq. ID No. 6428, Seq. ID No. 6430, Seq. ID No. 6432, Seq. ID No. 6454, Seq. ID No. 6466, Seq. ID No. 6470, Seq. ID No. 6472, Seq. ID No. 6480, Seq. ID No. 6540, Seq. ID No. 6542, Seq. ID No. 6572, Seq. ID No. 6574, Seq. ID No. 6640, Seq. ID No. 6644, and Seq. ID No. 6646.

In yet another preferred embodiment, the polypeptide is selected from the Alloiococcus otitidis polypeptides, which are predicted by HMM SignalP algorithm analysis as having a signal peptide and is selected from one of Seq. ID No. 4, Seq. ID No. 8, Seq. ID No. 10, Seq. ID No. 20, Seq. ID No. 22, Seq. ID No. 32, Seq. ID No. 34, Seq. ID No. 46, Seq. ID No. 48, Seq. ID No. 50, Seq. ID No. 54, Seq. ID No. 66, Seq. ID No. 68, Seq. ID No. 86, Seq. ID No. 88, Seq. ID No. 106, Seq. ID No. 112, Seq. ID No. 124, Seq. ID No. 132, Seq. ID No. 134, Seq. ID No. 142, Seq. ID No. 160, Seq. ID No. 162, Seq. ID No. 164, Seq. ID No. 172, Seq. ID No. 192, Seq. ID No. 210, Seq. ID No. 212, Seq. ID No. 214, Seq. ID No. 216, Seq. ID No. 218, Seq. ID No. 226, Seq. ID No. 236, Seq. ID No. 238, Seq. ID No. 250, Seq. ID No. 252, Seq. ID No. 256, Seq. ID No. 258, Seq. ID No. 260, Seq. ID No. 272, Seq. ID No. 274, Seq. ID No. 278, Seq. ID No. 286, Seq. ID No. 288, Seq. ID No. 290, Seq. ID No. 328, Seq. ID No. 330, Seq. ID No. 358, Seq. ID No. 360, Seq. ID No. 374, Seq. ID No. 376, Seq. ID No. 394, Seq. ID No. 402, Seq. ID No. 404, Seq. ID No. 456, Seq. ID No. 458, Seq. ID No. 478, Seq. ID No. 488, Seq. ID No. 490, Seq. ID No. 492, Seq. ID No. 494, Seq. ID No. 496, Seq. ID No. 498, Seq. ID No. 500, Seq. ID No. 504, Seq. ID No. 506, Seq. ID No. 510, Seq. ID No. 512, Seq. ID No. 514, Seq. ID No. 516, Seq. ID No. 526, Seq. ID No. 528, Seq. ID No. 546, Seq. ID No. 548, Seq. ID No. 550, Seq. ID No. 558, Seq. ID No. 568, Seq. ID No. 570, Seq. ID No. 576, Seq. ID No. 578, Seq. ID No. 580, Seq. ID No. 592, Seq. ID No. 594, Seq. ID No. 600, Seq. ID No. 618, Seq. ID No. 638, Seq. ID No. 640, Seq. ID No. 642, Seq. ID No. 644, Seq. ID No. 646, Seq. ID No. 648, Seq. ID No. 654, Seq. ID No. 656, Seq. ID No. 658, Seq. ID No. 660, Seq. ID No. 662, Seq. ID No. 668, Seq. ID No. 670, Seq. ID No. 676, Seq. ID No. 678, Seq. ID No. 680, Seq. ID No. 716, Seq. ID No. 718, Seq. ID No. 720, Seq. ID No. 722, Seq. ID No. 724, Seq. ID No. 726, Seq. ID No. 746, Seq. ID No. 748, Seq. ID No. 750, Seq. ID No. 752, Seq. ID No. 756, Seq. ID No. 758, Seq. ID No. 760, Seq. ID No. 762, Seq. ID No. 764, Seq. ID No. 766, Seq. ID No. 770, Seq. ID No. 778, Seq. ID No. 780, Seq. ID No. 782, Seq. ID No. 808, Seq. ID No. 810, Seq. ID No. 820, Seq. ID No. 826, Seq. ID No. 864, Seq. ID No. 894, Seq. ID No. 902, Seq. ID No. 936, Seq. ID No. 938, Seq. ID No. 942, Seq. ID No. 944, Seq. ID No. 956, Seq. ID No. 958, Seq. ID No. 968, Seq. ID No. 980, Seq. ID No. 982, Seq. ID No. 984, Seq. ID No. 986, Seq. ID No. 1002, Seq. ID No. 1004, Seq. ID No. 1006, Seq. ID No. 1008, Seq. ID No. 1010, Seq. ID No. 1012, Seq. ID No. 1014, Seq. ID No. 1016, Seq. ID No. 1020, Seq. ID No. 1030, Seq. ID No. 1032, Seq. ID No. 1034, Seq. ID No. 1036, Seq. ID No. 1038, Seq. ID No. 1040, Seq. ID No. 1046, Seq. ID No. 1056, Seq. ID No. 1076, Seq. ID No. 1078, Seq. ID No. 1106, Seq. ID No. 1108, Seq. ID No. 1132, Seq. ID No. 1136, Seq. ID No. 1138, Seq. ID No. 1140, Seq. ID No. 1152, Seq. ID No. 1166, Seq. ID No. 1168, Seq. ID No. 1170, Seq. ID No. 1174, Seq. ID No. 1184, Seq. ID No. 1196, Seq. ID No. 1198, Seq. ID No. 1204, Seq. ID No. 1214, Seq. ID No. 1224, Seq. ID No. 1228, Seq. ID No. 1230, Seq. ID No. 1238, Seq. ID No. 1244, Seq. ID No. 1246, Seq. ID No. 1260, Seq. ID No. 1266, Seq. ID No. 1268, Seq. ID No. 1270, Seq. ID No. 1272, Seq. ID No. 1284, Seq. ID No. 1288, Seq. ID No. 1290, Seq. ID No. 1292, Seq. ID No. 1296, Seq. ID No. 1298, Seq. ID No. 1304, Seq. ID No. 1306, Seq. ID No. 1316, Seq. ID No. 1318, Seq. ID No. 1320, Seq. ID No. 1326, Seq. ID No. 1328, Seq. ID No. 1330, Seq. ID No. 1332, Seq. ID No. 1338, Seq. ID No. 1342, Seq. ID No. 1344, Seq. ID No. 1348, Seq. ID No. 1350, Seq. ID No. 1352, Seq. ID No. 1354, Seq. ID No. 1366, Seq. ID No. 1368, Seq. ID No. 1370, Seq. ID No. 1372, Seq. ID No. 1382, Seq. ID No. 1402, Seq. ID No. 1430, Seq. ID No. 1436, Seq. ID No. 1446, Seq. ID No. 1448, Seq. ID No. 1482, Seq. ID No. 1514, Seq. ID No. 1516, Seq. ID No. 1518, Seq. ID No. 1530, Seq. ID No. 1534, Seq. ID No. 1536, Seq. ID No. 1546, Seq. ID No. 1548, Seq. ID No. 1552, Seq. ID No. 1554, Seq. ID No. 1560, Seq. ID No. 1562, Seq. ID No. 1564, Seq. ID No. 1572, Seq. ID No. 1580, Seq. ID No. 1584, Seq. ID No. 1586, Seq. ID No. 1622, Seq. ID No. 1634, Seq. ID No. 1640, Seq. ID No. 1644, Seq. ID No. 1646, Seq. ID No. 1648, Seq. ID No. 1650, Seq. ID No. 1654, Seq. ID No. 1662, Seq. ID No. 1664, Seq. ID No. 1666, Seq. ID No. 1672, Seq. ID No. 1674, Seq. ID No. 1678, Seq. ID No. 1680, Seq. ID No. 1682, Seq. ID No. 1694, Seq. ID No. 1708, Seq. ID No. 1712, Seq. ID No. 1732, Seq. ID No. 1734, Seq. ID No. 1738, Seq. ID No. 1754, Seq. ID No. 1756, Seq. ID No. 1758, Seq. ID No. 1766, Seq. ID No. 1768, Seq. ID No. 1772, Seq. ID No. 1782, Seq. ID No. 1788, Seq. ID No. 1792, Seq. ID No. 1798, Seq. ID No. 1806, Seq. ID No. 1820, Seq. ID No. 1822, Seq. ID No. 1824, Seq. ID No. 1840, Seq. ID No. 1858, Seq. ID No. 1870, Seq. ID No. 1876, Seq. ID No. 1892, Seq. ID No. 1894, Seq. ID No. 1896, Seq. ID No. 1916, Seq. ID No. 1918, Seq. ID No. 1920, Seq. ID No. 1924, Seq. ID No. 1928, Seq. ID No. 1930, Seq. ID No. 1970, Seq. ID No. 1982, Seq. ID No. 1992, Seq. ID No. 1994, Seq. ID No. 2006, Seq. ID No. 2014, Seq. ID No. 2018, Seq. ID No. 2020, Seq. ID No. 2026, Seq. ID No. 2028, Seq. ID No. 2034, Seq. ID No. 2036, Seq. ID No. 2042, Seq. ID No. 2050, Seq. ID No. 2054, Seq. ID No. 2056, Seq. ID No. 2088, Seq. ID No. 2090, Seq. ID No. 2092, Seq. ID No. 2106, Seq. ID No. 2108, Seq. ID No. 2110, Seq. ID No. 2114, Seq. ID No. 2116, Seq. ID No. 2122, Seq. ID No. 2124, Seq. ID No. 2126, Seq. ID No. 2128, Seq. ID No. 2150, Seq. ID No. 2152, Seq. ID No. 2156, Seq. ID No. 2158, Seq. ID No. 2160, Seq. ID No. 2172, Seq. ID No. 2174, Seq. ID No. 2178, Seq. ID No. 2188, Seq. ID No. 2192, Seq. ID No. 2206, Seq. ID No. 2238, Seq. ID No. 2240, Seq. ID No. 2246, Seq. ID No. 2250, Seq. ID No. 2252, Seq. ID No. 2256, Seq. ID No. 2260, Seq. ID No. 2272, Seq. ID No. 2284, Seq. ID No. 2290, Seq. ID No. 2292, Seq. ID No. 2294, Seq. ID No. 2296, Seq. ID No. 2304, Seq. ID No. 2306, Seq. ID No. 2308, Seq. ID No. 2318, Seq. ID No. 2320, Seq. ID No. 2322, Seq. ID No. 2324, Seq. ID No. 2326, Seq. ID No. 2328, Seq. ID No. 2330, Seq. ID No. 2334, Seq. ID No. 2344, Seq. ID No. 2348, Seq. ID No. 2350, Seq. ID No. 2352, Seq. ID No. 2354, Seq. ID No. 2356, Seq. ID No. 2358, Seq. ID No. 2366, Seq. ID No. 2368, Seq. ID No. 2372, Seq. ID No. 2376, Seq. ID No. 2388, Seq. ID No. 2390, Seq. ID No. 2392, Seq. ID No. 2394, Seq. ID No. 2408, Seq. ID No. 2420, Seq. ID No. 2422, Seq. ID No. 2426, Seq. ID No. 2428, Seq. ID No. 2430, Seq. ID No. 2434, Seq. ID No. 2450, Seq. ID No. 2460, Seq. ID No. 2488, Seq. ID No. 2490, Seq. ID No. 2492, Seq. ID No. 2494, Seq. ID No. 2498, Seq. ID No. 2500, Seq. ID No. 2502, Seq. ID No. 2504, Seq. ID No. 2508, Seq. ID No. 2516, Seq. ID No. 2518, Seq. ID No. 2524, Seq. ID No. 2526, Seq. ID No. 2528, Seq. ID No. 2532, Seq. ID No. 2534, Seq. ID No. 2536, Seq. ID No. 2538, Seq. ID No. 2542, Seq. ID No. 2544, Seq. ID No. 2546, Seq. ID No. 2556, Seq. ID No. 2558, Seq. ID No. 2566, Seq. ID No. 2570, Seq. ID No. 2572, Seq. ID No. 2584, Seq. ID No. 2592, Seq. ID No. 2598, Seq. ID No. 2600, Seq. ID No. 2602, Seq. ID No. 2608, Seq. ID No. 2612, Seq. ID No. 2622, Seq. ID No. 2628, Seq. ID No. 2630, Seq. ID No. 2652, Seq. ID No. 2680, Seq. ID No. 2682, Seq. ID No. 2684, Seq. ID No. 2692, Seq. ID No. 2694, Seq. ID No. 2696, Seq. ID No. 2698, Seq. ID No. 2700, Seq. ID No. 2702, Seq. ID No. 2704, Seq. ID No. 2706, Seq. ID No. 2708, Seq. ID No. 2718, Seq. ID No. 2730, Seq. ID No. 2732, Seq. ID No. 2734, Seq. ID No. 2784, Seq. ID No. 2786, Seq. ID No. 2802, Seq. ID No. 2810, Seq. ID No. 2814, Seq. ID No. 2816, Seq. ID No. 2820, Seq. ID No. 2822, Seq. ID No. 2824, Seq. ID No. 2832, Seq. ID No. 2834, Seq. ID No. 2836, Seq. ID No. 2838, Seq. ID No. 2840, Seq. ID No. 2842, Seq. ID No. 2846, Seq. ID No. 2854, Seq. ID No. 2856, Seq. ID No. 2860, Seq. ID No. 2862, Seq. ID No. 2874, Seq. ID No. 2882, Seq. ID No. 2894, Seq. ID No. 2906, Seq. ID No. 2936, Seq. ID No. 2938, Seq. ID No. 2940, Seq. ID No. 2942, Seq. ID No. 2948, Seq. ID No. 2950, Seq. ID No. 2952, Seq. ID No. 2970, Seq. ID No. 2974, Seq. ID No. 2976, Seq. ID No. 2978, Seq. ID No. 2982, Seq. ID No. 2984, Seq. ID No. 2988, Seq. ID No. 2992, Seq. ID No. 2996, Seq. ID No. 2998, Seq. ID No. 3006, Seq. ID No. 3008, Seq. ID No. 3010, Seq. ID No. 3012, Seq. ID No. 3014, Seq. ID No. 3024, Seq. ID No. 3026, Seq. ID No. 3028, Seq. ID No. 3030, Seq. ID No. 3032, Seq. ID No. 3044, Seq. ID No. 3056, Seq. ID No. 3064, Seq. ID No. 3072, Seq. ID No. 3074, Seq. ID No. 3076, Seq. ID No. 3078, Seq. ID No. 3120, Seq. ID No. 3122, Seq. ID No. 3124, Seq. ID No. 3146, Seq. ID No. 3156, Seq. ID No. 3158, Seq. ID No. 3164, Seq. ID No. 3166, Seq. ID No. 3174, Seq. ID No. 3184, Seq. ID No. 3194, Seq. ID No. 3202, Seq. ID No. 3214, Seq. ID No. 3216, Seq. ID No. 3218, Seq. ID No. 3220, Seq. ID No. 3242, Seq. ID No. 3244, Seq. ID No. 3246, Seq. ID No. 3250, Seq. ID No. 3256, Seq. ID No. 3258, Seq. ID No. 3262, Seq. ID No. 3264, Seq. ID No. 3270, Seq. ID No. 3276, Seq. ID No. 3278, Seq. ID No. 3298, Seq. ID No. 3318, Seq. ID No. 3324, Seq. ID No. 3326, Seq. ID No. 3332, Seq. ID No. 3336, Seq. ID No. 3338, Seq. ID No. 3340, Seq. ID No. 3350, Seq. ID No. 3352, Seq. ID No. 3354, Seq. ID No. 3356, Seq. ID No. 3358, Seq. ID No. 3360, Seq. ID No. 3376, Seq. ID No. 3378, Seq. ID No. 3390, Seq. ID No. 3392, Seq. ID No. 3396, Seq. ID No. 3406, Seq. ID No. 3416, Seq. ID No. 3422, Seq. ID No. 3426, Seq. ID No. 3428, Seq. ID No. 3434, Seq. ID No. 3442, Seq. ID No. 3450, Seq. ID No. 3452, Seq. ID No. 3454, Seq. ID No. 3456, Seq. ID No. 3458, Seq. ID No. 3464, Seq. ID No. 3482, Seq. ID No. 3492, Seq. ID No. 3494, Seq. ID No. 3504, Seq. ID No. 3506, Seq. ID No. 3518, Seq. ID No. 3520, Seq. ID No. 3528, Seq. ID No. 3532, Seq. ID No. 3540, Seq. ID No. 3562, Seq. ID No. 3574, Seq. ID No. 3582, Seq. ID No. 3588, Seq. ID No. 3594, Seq. ID No. 3596, Seq. ID No. 3612, Seq. ID No. 3618, Seq. ID No. 3620, Seq. ID No. 3622, Seq. ID No. 3626, Seq. ID No. 3630, Seq. ID No. 3636, Seq. ID No. 3678, Seq. ID No. 3680, Seq. ID No. 3690, Seq. ID No. 3718, Seq. ID No. 3724, Seq. ID No. 3728, Seq. ID No. 3742, Seq. ID No. 3746, Seq. ID No. 3754, Seq. ID No. 3756, Seq. ID No. 3758, Seq. ID No. 3762, Seq. ID No. 3764, Seq. ID No. 3766, Seq. ID No. 3782, Seq. ID No. 3784, Seq. ID No. 3794, Seq. ID No. 3800, Seq. ID No. 3808, Seq. ID No. 3814, Seq. ID No. 3816, Seq. ID No. 3818, Seq. ID No. 3838, Seq. ID No. 3840, Seq. ID No. 3850, Seq. ID No. 3852, Seq. ID No. 3854, Seq. ID No. 3860, Seq. ID No. 3862, Seq. ID No. 3864, Seq. ID No. 3866, Seq. ID No. 3880, Seq. ID No. 3890, Seq. ID No. 3892, Seq. ID No. 3896, Seq. ID No. 3904, Seq. ID No. 3916, Seq. ID No. 3924, Seq. ID No. 3926, Seq. ID No. 3960, Seq. ID No. 3964, Seq. ID No. 3974, Seq. ID No. 3976, Seq. ID No. 3994, Seq. ID No. 3996, Seq. ID No. 3998, Seq. ID No. 4006, Seq. ID No. 4008, Seq. ID No. 4010, Seq. ID No. 4026, Seq. ID No. 4028, Seq. ID No. 4040, Seq. ID No. 4042, Seq. ID No. 4044, Seq. ID No. 4046, Seq. ID No. 4048, Seq. ID No. 4050, Seq. ID No. 4062, Seq. ID No. 4072, Seq. ID No. 4074, Seq. ID No. 4080, Seq. ID No. 4082, Seq. ID No. 4084, Seq. ID No. 4102, Seq. ID No. 4104, Seq. ID No. 4112, Seq. ID No. 4134, Seq. ID No. 4136, Seq. ID No. 4138, Seq. ID No. 4140, Seq. ID No. 4142, Seq. ID No. 4144, Seq. ID No. 4146, Seq. ID No. 4152, Seq. ID No. 4154, Seq. ID No. 4182, Seq. ID No. 4222, Seq. ID No. 4224, Seq. ID No. 4226, Seq. ID No. 4228, Seq. ID No. 4232, Seq. ID No. 4234, Seq. ID No. 4242, Seq. ID No. 4248, Seq. ID No. 4254, Seq. ID No. 4258, Seq. ID No. 4260, Seq. ID No. 4262, Seq. ID No. 4264, Seq. ID No. 4266, Seq. ID No. 4270, Seq. ID No. 4276, Seq. ID No. 4286, Seq. ID No. 4288, Seq. ID No. 4298, Seq. ID No. 4308, Seq. ID No. 4312, Seq. ID No. 4314, Seq. ID No. 4344, Seq. ID No. 4380, Seq. ID No. 4386, Seq. ID No. 4390, Seq. ID No. 4392, Seq. ID No. 4400, Seq. ID No. 4404, Seq. ID No. 4438, Seq. ID No. 4440, Seq. ID No. 4442, Seq. ID No. 4470, Seq. ID No. 4474, Seq. ID No. 4482, Seq. ID No. 4484, Seq. ID No. 4486, Seq. ID No. 4494, Seq. ID No. 4496, Seq. ID No. 4498, Seq. ID No. 4514, Seq. ID No. 4516, Seq. ID No. 4528, Seq. ID No. 4530, Seq. ID No. 4534, Seq. ID No. 4588, Seq. ID No. 4590, Seq. ID No. 4640, Seq. ID No. 4642, Seq. ID No. 4644, Seq. ID No. 4660, Seq. ID No. 4682, Seq. ID No. 4688, Seq. ID No. 4696, Seq. ID No. 4706, Seq. ID No. 4708, Seq. ID No. 4710, Seq. ID No. 4712, Seq. ID No. 4716, Seq. ID No. 4718, Seq. ID No. 4738, Seq. ID No. 4760, Seq. ID No. 4814, Seq. ID No. 4828, Seq. ID No. 4854, Seq. ID No. 4864, Seq. ID No. 4870, Seq. ID No. 4872, Seq. ID No. 4874, Seq. ID No. 4876, Seq. ID No. 4920, Seq. ID No. 4922, Seq. ID No. 4924, Seq. ID No. 4930, Seq. ID No. 4932, Seq. ID No. 4950, Seq. ID No. 4952, Seq. ID No. 4976, Seq. ID No. 4988, Seq. ID No. 5000, Seq. ID No. 5014, Seq. ID No. 5032, Seq. ID No. 5062, Seq. ID No. 5064, Seq. ID No. 5070, Seq. ID No. 5086, Seq. ID No. 5104, Seq. ID No. 5116, Seq. ID No. 5124, Seq. ID No. 5126, Seq. ID No. 5130, Seq. ID No. 5132, Seq. ID No. 5134, Seq. ID No. 5136, Seq. ID No. 5138, Seq. ID No. 5140, Seq. ID No. 5142, Seq. ID No. 5160, Seq. ID No. 5166, Seq. ID No. 5176, Seq. ID No. 5198, Seq. ID No. 5200, Seq. ID No. 5206, Seq. ID No. 5208, Seq. ID No. 5214, Seq. ID No. 5220, Seq. ID No. 5224, Seq. ID No. 5268, Seq. ID No. 5274, Seq. ID No. 5276, Seq. ID No. 5278, Seq. ID No. 5280, Seq. ID No. 5282, Seq. ID No. 5286, Seq. ID No. 5328, Seq. ID No. 5330, Seq. ID No. 5332, Seq. ID No. 5342, Seq. ID No. 5378, Seq. ID No. 5380, Seq. ID No. 5386, Seq. ID No. 5388, Seq. ID No. 5406, Seq. ID No. 5414, Seq. ID No. 5416, Seq. ID No. 5434, Seq. ID No. 5454, Seq. ID No. 5460, Seq. ID No. 5462, Seq. ID No. 5464, Seq. ID No. 5466, Seq. ID No. 5492, Seq. ID No. 5498, Seq. ID No. 5510, Seq. ID No. 5514, Seq. ID No. 5516, Seq. ID No. 5518, Seq. ID No. 5526, Seq. ID No. 5530, Seq. ID No. 5532, Seq. ID No. 5534, Seq. ID No. 5544, Seq. ID No. 5546, Seq. ID No. 5570, Seq. ID No. 5588, Seq. ID No. 5590, Seq. ID No. 5592, Seq. ID No. 5594, Seq. ID No. 5596, Seq. ID No. 5598, Seq. ID No. 5610, Seq. ID No. 5612, Seq. ID No. 5622, Seq. ID No. 5624, Seq. ID No. 5626, Seq. ID No. 5634, Seq. ID No. 5638, Seq. ID No. 5640, Seq. ID No. 5642, Seq. ID No. 5644, Seq. ID No. 5650, Seq. ID No. 5660, Seq. ID No. 5668, Seq. ID No. 5682, Seq. ID No. 5684, Seq. ID No. 5686, Seq. ID No. 5688, Seq. ID No. 5690, Seq. ID No. 5700, Seq. ID No. 5704, Seq. ID No. 5706, Seq. ID No. 5710, Seq. ID No. 5712, Seq. ID No. 5720, Seq. ID No. 5730, Seq. ID No. 5734, Seq. ID No. 5736, Seq. ID No. 5740, Seq. ID No. 5764, Seq. ID No. 5766, Seq. ID No. 5768, Seq. ID No. 5772, Seq. ID No. 5784, Seq. ID No. 5786, Seq. ID No. 5792, Seq. ID No. 5798, Seq. ID No. 5830, Seq. ID No. 5880, Seq. ID No. 5884, Seq. ID No. 5886, Seq. ID No. 5890, Seq. ID No. 5894, Seq. ID No. 5896, Seq. ID No. 5900, Seq. ID No. 5902, Seq. ID No. 5906, Seq. ID No. 5910, Seq. ID No. 5922, Seq. ID No. 5924, Seq. ID No. 5928, Seq. ID No. 5930, Seq. ID No. 5940, Seq. ID No. 6024, Seq. ID No. 6026, Seq. ID No. 6028, Seq. ID No. 6044, Seq. ID No. 6048, Seq. ID No. 6054, Seq. ID No. 6060, Seq. ID No. 6064, Seq. ID No. 6068, Seq. ID No. 6088, Seq. ID No. 6090, Seq. ID No. 6092, Seq. ID No. 6096, Seq. ID No. 6104, Seq. ID No. 6108, Seq. ID No. 6110, Seq. ID No. 6112, Seq. ID No. 6116, Seq. ID No. 6118, Seq. ID No. 6136, Seq. ID No. 6138, Seq. ID No. 6160, Seq. ID No. 6162, Seq. ID No. 6164, Seq. ID No. 6166, Seq. ID No. 6170, Seq. ID No. 6184, Seq. ID No. 6186, Seq. ID No. 6188, Seq. ID No. 6190, Seq. ID No. 6192, Seq. ID No. 6198, Seq. ID No. 6204, Seq. ID No. 6206, Seq. ID No. 6210, Seq. ID No. 6218, Seq. ID No. 6222, Seq. ID No. 6224, Seq. ID No. 6226, Seq. ID No. 6228, Seq. ID No. 6230, Seq. ID No. 6236, Seq. ID No. 6238, Seq. ID No. 6242, Seq. ID No. 6244, Seq. ID No. 6246, Seq. ID No. 6250, Seq. ID No. 6254, Seq. ID No. 6260, Seq. ID No. 6264, Seq. ID No. 6266, Seq. ID No. 6268, Seq. ID No. 6274, Seq. ID No. 6276, Seq. ID No. 6284, Seq. ID No. 6286, Seq. ID No. 6290, Seq. ID No. 6302, Seq. ID No. 6304, Seq. ID No. 6326, Seq. ID No. 6328, Seq. ID No. 6364, Seq. ID No. 6366, Seq. ID No. 6378, Seq. ID No. 6382, Seq. ID No. 6384, Seq. ID No. 6416, Seq. ID No. 6426, Seq. ID No. 6428, Seq. ID No. 6430, Seq. ID No. 6432, Seq. ID No. 6474, Seq. ID No. 6480, Seq. ID No. 6498, Seq. ID No. 6518, Seq. ID No. 6542, Seq. ID No. 6544, Seq. ID No. 6556, Seq. ID No. 6558, Seq. ID No. 6572, Seq. ID No. 6574, Seq. ID No. 6582, Seq. ID No. 6594, Seq. ID No. 6596, Seq. ID No. 6598, Seq. ID No. 6600, Seq. ID No. 6602, Seq. ID No. 6604, Seq. ID No. 6632, Seq. ID No. 6638, Seq. ID No. 6640, Seq. ID No. 6644, Seq. ID No. 6646, and Seq. ID No. 6650.

In yet another embodiment, the Alloiococcus otitidis polypeptide is one that is predicted by HMM SignalP algorithm analysis as being a non-secretory protein, and is selected from one of Seq. ID No. 2, Seq. ID No. 6, Seq. ID No. 12, Seq. ID No. 14, Seq. ID No. 16, Seq. ID No. 18, Seq. ID No. 24, Seq. ID No. 26, Seq. ID No. 28, Seq. ID No. 30, Seq. ID No. 36, Seq. ID No. 38, Seq. ID No. 40, Seq. ID No. 42, Seq. ID No. 44, Seq. ID No. 52, Seq. ID No. 56, Seq. ID No. 58, Seq. ID No. 60, Seq. ID No. 62, Seq. ID No. 64, Seq. ID No. 70, Seq. ID No. 72, Seq. ID No. 74, Seq. ID No. 76, Seq. ID No. 78, Seq. ID No. 80, Seq. ID No. 82, Seq. ID No. 84, Seq. ID No. 90, Seq. ID No. 92, Seq. ID No. 94, Seq. ID No. 96, Seq. ID No. 98, Seq. ID No. 100, Seq. ID No. 102, Seq. ID No. 104, Seq. ID No. 108, Seq. ID No. 110, Seq. ID No. 114, Seq. ID No. 116, Seq. ID No. 118, Seq. ID No. 120, Seq. ID No. 122, Seq. ID No. 126, Seq. ID No. 128, Seq. ID No. 130, Seq. ID No. 136, Seq. ID No. 138, Seq. ID No. 140, Seq. ID No. 144, Seq. ID No. 146, Seq. ID No. 148, Seq. ID No. 150, Seq. ID No. 152, Seq. ID No. 154, Seq. ID No. 156, Seq. ID No. 158, Seq. ID No. 166, Seq. ID No. 168, Seq. ID No. 170, Seq. ID No. 174, Seq. ID No. 176, Seq. ID No. 178, Seq. ID No. 180, Seq. ID No. 182, Seq. ID No. 184, Seq. ID No. 186, Seq. ID No. 188, Seq. ID No. 190, Seq. ID No. 194, Seq. ID No. 196, Seq. ID No. 198, Seq. ID No. 200, Seq. ID No. 202, Seq. ID No. 204, Seq. ID No. 206, Seq. ID No. 208, Seq. ID No. 220, Seq. ID No. 222, Seq. ID No. 224, Seq. ID No. 228, Seq. ID No. 230, Seq. ID No. 232, Seq. ID No. 234, Seq. ID No. 240, Seq. ID No. 242, Seq. ID No. 244, Seq. ID No. 246, Seq. ID No. 248, Seq. ID No. 254, Seq. ID No. 262, Seq. ID No. 264, Seq. ID No. 266, Seq. ID No. 268, Seq. ID No. 270, Seq. ID No. 276, Seq. ID No. 280, Seq. ID No. 282, Seq. ID No. 284, Seq. ID No. 292, Seq. ID No. 294, Seq. ID No. 296, Seq. ID No. 298, Seq. ID No. 300, Seq. ID No. 302, Seq. ID No. 304, Seq. ID No. 306, Seq. ID No. 308, Seq. ID No. 310, Seq. ID No. 312, Seq. ID No. 314, Seq. ID No. 316, Seq. ID No. 318, Seq. ID No. 320, Seq. ID No. 322; Seq. ID No. 324, Seq. ID No. 326, Seq. ID No. 332, Seq. ID No. 334, Seq. ID No. 336, Seq. ID No. 338, Seq. ID No. 340, Seq. ID No. 342, Seq. ID No. 344, Seq. ID No. 346, Seq. ID No. 348, Seq. ID No. 350, Seq. ID No. 352, Seq. ID No. 354, Seq. ID No. 356, Seq. ID No. 362, Seq. ID No. 364, Seq. ID No. 366, Seq. ID No. 368, Seq. ID No. 370, Seq. ID No. 372, Seq. ID No. 378, Seq. ID No. 380, Seq. ID No. 382, Seq. ID No. 384, Seq. ID No. 386, Seq. ID No. 388, Seq. ID No. 390, Seq. ID No. 392, Seq. ID No. 396, Seq. ID No. 398, Seq. ID No. 400, Seq. ID No. 406, Seq. ID No. 408, Seq. ID No. 410, Seq. ID No. 412, Seq. ID No. 414, Seq. ID No. 416, Seq. ID No. 418, Seq. ID No. 420, Seq. ID No. 422, Seq. ID No. 424, Seq. ID No. 426, Seq. ID No. 428, Seq. ID No. 430, Seq. ID No. 432, Seq. ID No. 434, Seq. ID No. 436, Seq. ID No. 438, Seq. ID No. 440, Seq. ID No. 442, Seq. ID No. 444, Seq. ID No. 446, Seq. ID No. 448, Seq. ID No. 450, Seq. ID No. 452, Seq. ID No. 454, Seq. ID No. 460, Seq. ID No. 462, Seq. ID No. 464, Seq. ID No. 466, Seq. ID No. 468, Seq. ID No. 470, Seq. ID No. 472, Seq. ID No. 474, Seq. ID No. 476, Seq. ID No. 480, Seq. ID No. 482, Seq. ID No. 484, Seq. ID No. 486, Seq. ID No. 502, Seq. ID No. 508, Seq. ID No. 518, Seq. ID No. 520, Seq. ID No. 522, Seq. ID No. 524, Seq. ID No. 530, Seq. ID No. 532, Seq. ID No. 534, Seq. ID No. 536, Seq. ID No. 538, Seq. ID No. 540, Seq. ID No. 542, Seq. ID No. 544, Seq. ID No. 552, Seq. ID No. 554, Seq. ID No. 556, Seq. ID No. 560, Seq. ID No. 562, Seq. ID No. 564, Seq. ID No. 566, Seq. ID No. 572, Seq. ID No. 574, Seq. ID No. 582, Seq. ID No. 584, Seq. ID No. 586, Seq. ID No. 588, Seq. ID No. 590, Seq. ID No. 596, Seq. ID No. 598, Seq. ID No. 602, Seq. ID No. 604, Seq. ID No. 606, Seq. ID No. 608, Seq. ID No. 610, Seq. ID No. 612, Seq. ID No. 614, Seq. ID No. 616, Seq. ID No. 620, Seq. ID No. 622, Seq. ID No. 624, Seq. ID No. 626, Seq. ID No. 628, Seq. ID No. 630, Seq. ID No. 632, Seq. ID No. 634, Seq. ID No. 636, Seq. ID No. 650, Seq. ID No. 652, Seq. ID No. 664, Seq. ID No. 666, Seq. ID No. 672, Seq. ID No. 674, Seq. ID No. 682, Seq. ID No. 684, Seq. ID No. 686, Seq. ID No. 688, Seq. ID No. 690, Seq. ID No. 692, Seq. ID No. 694, Seq. ID No. 696, Seq. ID No. 698, Seq. ID No. 700, Seq. ID No. 702, Seq. ID No. 704, Seq. ID No. 706, Seq. ID No. 708, Seq. ID No. 710, Seq. ID No. 712, Seq. ID No. 714, Seq. ID No. 728, Seq. ID No. 730, Seq. ID No. 732, Seq. ID No. 734, Seq. ID No. 736, Seq. ID No. 738, Seq. ID No. 740, Seq. ID No. 742, Seq. ID No. 744, Seq. ID No. 754, Seq. ID No. 768, Seq. ID No. 772, Seq. ID No. 774, Seq. ID No. 776, Seq. ID No. 784, Seq. ID No. 786, Seq. ID No. 788, Seq. ID No. 790, Seq. ID No. 792, Seq. ID No. 794, Seq. ID No. 796, Seq. ID No. 798, Seq. ID No. 800, Seq. ID No. 802, Seq. ID No. 804, Seq. ID No. 806, Seq. ID No. 812, Seq. ID No. 814, Seq. ID No. 816, Seq. ID No. 818, Seq. ID No. 822, Seq. ID No. 824, Seq. ID No. 828, Seq. ID No. 830, Seq. ID No. 832, Seq. ID No. 834, Seq. ID No. 836, Seq. ID No. 838, Seq. ID No. 840, Seq. ID No. 842, Seq. ID No. 844, Seq. ID No. 846, Seq. ID No. 848, Seq. ID No. 850, Seq. ID No. 852, Seq. ID No. 854, Seq. ID No. 856, Seq. ID No. 858, Seq. ID No. 860, Seq. ID No. 862, Seq. ID No. 866, Seq. ID No. 868, Seq. ID No. 870, Seq. ID No. 872, Seq. ID No. 874, Seq. ID No. 876, Seq. ID No. 878, Seq. ID No. 880, Seq. ID No. 882, Seq. ID No. 884, Seq. ID No. 886, Seq. ID No. 888, Seq. ID No. 890, Seq. ID No. 892, Seq. ID No. 896, Seq. ID No. 898, Seq. ID No. 900, Seq. ID No. 904, Seq. ID No. 906, Seq. ID No. 908, Seq. ID No. 910, Seq. ID No. 912, Seq. ID No. 914, Seq. ID No. 916, Seq. ID No. 918, Seq. ID No. 920, Seq. ID No. 922, Seq. ID No. 924, Seq. ID No. 926, Seq. ID No. 928, Seq. ID No. 930, Seq. ID No. 932, Seq. ID No. 934, Seq. ID No. 940, Seq. ID No. 946, Seq. ID No. 948, Seq. ID No. 950, Seq. ID No. 952, Seq. ID No. 954, Seq. ID No. 960, Seq. ID No. 962, Seq. ID No. 964, Seq. ID No. 966, Seq. ID No. 970, Seq. ID No. 972, Seq. ID No. 974, Seq. ID No. 976, Seq. ID No. 978, Seq. ID No. 988, Seq. ID No. 990, Seq. ID No. 992, Seq. ID No. 994, Seq. ID No. 996, Seq. ID No. 998, Seq. ID No. 1000, Seq. ID No. 1018, Seq. ID No. 1022, Seq. ID No. 1024, Seq. ID No. 1026, Seq. ID No. 1028, Seq. ID No. 1042, Seq. ID No. 1044, Seq. ID No. 1048, Seq. ID No. 1050, Seq. ID No. 1052, Seq. ID No. 1054, Seq. ID No. 1058, Seq. ID No. 1060, Seq. ID No. 1062, Seq. ID No. 1064, Seq. ID No. 1066, Seq. ID No. 1068, Seq. ID No. 1070, Seq. ID No. 1072, Seq. ID No. 1074, Seq. ID No. 1080, Seq. ID No. 1082, Seq. ID No. 1084, Seq. ID No. 1086, Seq. ID No. 1088, Seq. ID No. 1090, Seq. ID No. 1092, Seq. ID No. 1094, Seq. ID No. 1096, Seq. ID No. 1098, Seq. ID No. 1100, Seq. ID No. 1102, Seq. ID No. 1104, Seq. ID No. 1110, Seq. ID No. 1112, Seq. ID No. 1114, Seq. ID No. 1116, Seq. ID No. 1118, Seq. ID No. 1120, Seq. ID No. 1122, Seq. ID No. 1124, Seq. ID No. 1126, Seq. ID No. 1128, Seq. ID No. 1130, Seq. ID No. 1134, Seq. ID No. 1142, Seq. ID No. 1144, Seq. ID No. 1146, Seq. ID No. 1148, Seq. ID No. 1150, Seq. ID No. 1154, Seq. ID No. 1156, Seq. ID No. 1158, Seq. ID No. 1160, Seq. ID No. 1162, Seq. ID No. 1164, Seq. ID No. 1172, Seq. ID No. 1176, Seq. ID No. 1178, Seq. ID No. 1180, Seq. ID No. 1182, Seq. ID No. 1186, Seq. ID No. 1188, Seq. ID No. 1190, Seq. ID No. 1192, Seq. ID No. 1194, Seq. ID No. 1200, Seq. ID No. 1202, Seq. ID No. 1206, Seq. ID No. 1208, Seq. ID No. 1210, Seq. ID No. 1212, Seq. ID No. 1216, Seq. ID No. 1218, Seq. ID No. 1220, Seq. ID No. 1222, Seq. ID No. 1226, Seq. ID No. 1232, Seq. ID No. 1234, Seq. ID No. 1236, Seq. ID No. 1240, Seq. ID No. 1242, Seq. ID No. 1248, Seq. ID No. 1250, Seq. ID No. 1252, Seq. ID No. 1254, Seq. ID No. 1256, Seq. ID No. 1258, Seq. ID No. 1262, Seq. ID No. 1264, Seq. ID No. 1274, Seq. ID No. 1276, Seq. ID No. 1278, Seq. ID No. 1280, Seq. ID No. 1282, Seq. ID No. 1286, Seq. ID No. 1294, Seq. ID No. 1300, Seq. ID No. 1302, Seq. ID No. 1308, Seq. ID No. 1310, Seq. ID No. 1312, Seq. ID No. 1314, Seq. ID No. 1322, Seq. ID No. 1324, Seq. ID No. 1334, Seq. ID No. 1336, Seq. ID No. 1340, Seq. ID No. 1346, Seq. ID No. 1356, Seq. ID No. 1358, Seq. ID No. 1360, Seq. ID No. 1362, Seq. ID No. 1364, Seq. ID No. 1374, Seq. ID No. 1376, Seq. ID No. 1378, Seq. ID No. 1380, Seq. ID No. 1384, Seq. ID No. 1386, Seq. ID No. 1388, Seq. ID No. 1390, Seq. ID No. 1392, Seq. ID No. 1394, Seq. ID No. 1396, Seq. ID No. 1398, Seq. ID No. 1400, Seq. ID No. 1404, Seq. ID No. 1406, Seq. ID No. 1408, Seq. ID No. 1410, Seq. ID No. 1412, Seq. ID No. 1414, Seq. ID No. 1416, Seq. ID No. 1418, Seq. ID No. 1420, Seq. ID No. 1422, Seq. ID No. 1424, Seq. ID No. 1426, Seq. ID No. 1428, Seq. ID No. 1432, Seq. ID No. 1434, Seq. ID No. 1438, Seq. ID No. 1440, Seq. ID No. 1442, Seq. ID No. 1444, Seq. ID No. 1450, Seq. ID No. 1452, Seq. ID No. 1454, Seq. ID No. 1456, Seq. ID No. 1458, Seq. ID No. 1460, Seq. ID No. 1462, Seq. ID No. 1464, Seq. ID No. 1466, Seq. ID No. 1468, Seq. ID No. 1470, Seq. ID No. 1472, Seq. ID No. 1474, Seq. ID No. 1476, Seq. ID No. 1478, Seq. ID No. 1480, Seq. ID No. 1484, Seq. ID No. 1486, Seq. ID No. 1488, Seq. ID No. 1490, Seq. ID No. 1492, Seq. ID No. 1494, Seq. ID No. 1496, Seq. ID No. 1498, Seq. ID No. 1500, Seq. ID No. 1502, Seq. ID No. 1504, Seq. ID No. 1506, Seq. ID No. 1508, Seq. ID No. 1510, Seq. ID No. 1512, Seq. ID No. 1520, Seq. ID No. 1522, Seq. ID No. 1524, Seq.-ID No. 1526, Seq. ID No. 1528, Seq. ID No. 1532, Seq. ID No. 1538, Seq. ID No. 1540, Seq. ID No. 1542, Seq. ID No. 1544, Seq. ID No. 1550, Seq. ID No. 1556, Seq. ID No. 1558, Seq. ID No. 1566, Seq. ID No. 1568, Seq. ID No. 1570, Seq. ID No. 1574, Seq. ID No. 1576, Seq. ID No. 1578, Seq. ID No. 1582, Seq. ID No. 1588, Seq. ID No. 1590, Seq. ID No. 1592, Seq. ID No. 1594, Seq. ID No. 1596, Seq. ID No. 1598, Seq. ID No. 1602, Seq. ID No. 1604, Seq. ID No. 1606, Seq. ID No. 1608, Seq. ID No. 1610, Seq. ID No. 1612, Seq. ID No. 1614, Seq. ID No. 1616, Seq. ID No. 1618, Seq. ID No. 1620, Seq. ID No. 1624, Seq. ID No. 1626, Seq. ID No. 1628, Seq. ID No. 1630, Seq. ID No. 1632, Seq. ID No. 1636, Seq. ID No. 1638, Seq. ID No. 1642, Seq. ID No. 1652, Seq. ID No. 1656, Seq. ID No. 1658, Seq. ID No. 1660, Seq. ID No. 1668, Seq. ID No. 1670, Seq. ID No. 1676, Seq. ID No. 1684, Seq. ID No. 1686, Seq. ID No. 1688, Seq. ID No. 1690, Seq. ID No. 1692, Seq. ID No. 1696, Seq. ID No. 1698, Seq. ID No. 1700, Seq. ID No. 1702, Seq. ID No. 1704, Seq. ID No. 1706, Seq. ID No. 1710, Seq. ID No. 1714, Seq. ID No. 1716, Seq. ID No. 1718, Seq. ID No. 1720, Seq. ID No. 1722, Seq. ID No. 1724, Seq. ID No. 1726, Seq. ID No. 1728, Seq. ID No. 1730, Seq. ID No. 1736, Seq. ID No. 1740, Seq. ID No. 1742, Seq. ID No. 1744, Seq. ID No. 1746, Seq. ID No. 1748, Seq. ID No. 1750, Seq. ID No. 1752, Seq. ID No. 1760, Seq. ID No. 1762, Seq. ID No. 1764, Seq. ID No. 1770, Seq. ID No. 1774, Seq. ID No. 1776, Seq. ID No. 1778, Seq. ID No. 1780, Seq. ID No. 1784, Seq. ID No. 1786, Seq. ID No. 1790, Seq. ID No. 1794, Seq. ID No. 1796, Seq. ID No. 1800, Seq. ID No. 1802, Seq. ID No. 1804, Seq. ID No. 1808, Seq. ID No. 1810, Seq. ID No. 1812, Seq. ID No. 1814, Seq. ID No. 1816, Seq. ID No. 1818, Seq. ID No. 1826, Seq. ID No. 1828, Seq. ID No. 1830, Seq. ID No. 1832, Seq. ID No. 1834, Seq. ID No. 1836, Seq. ID No. 1838, Seq. ID No. 1842, Seq. ID No. 1844, Seq. ID No. 1846, Seq. ID No. 1848, Seq. ID No. 1850, Seq. ID No. 1852, Seq. ID No. 1854, Seq. ID No. 1856, Seq. ID No. 1860, Seq. ID No. 1862, Seq. ID No. 1864, Seq. ID No. 1866, Seq. ID No. 1868, Seq. ID No. 1872, Seq. ID No. 1874, Seq. ID No. 1878, Seq. ID No. 1880, Seq. ID No. 1882, Seq. ID No. 1884, Seq. ID No. 1886, Seq. ID No. 1888, Seq. ID No. 1890, Seq. ID No. 1898, Seq. ID No. 1900, Seq. ID No. 1902, Seq. ID No. 1904, Seq. ID No. 1906, Seq. ID No. 1908, Seq. ID No. 1910, Seq. ID No. 1912, Seq. ID No. 1914, Seq. ID No. 1922, Seq. ID No. 1926, Seq. ID No. 1932, Seq. ID No. 1934, Seq. ID No. 1936, Seq. ID No. 1938, Seq. ID No. 1940, Seq. ID No. 1942, Seq. ID No. 1944, Seq. ID No. 1946, Seq. ID No. 1948, Seq. ID No. 1950, Seq. ID No. 1952, Seq. ID No. 1954, Seq. ID No. 1956, Seq. ID No. 1958, Seq. ID No. 1960, Seq. ID No. 1962, Seq. ID No. 1964, Seq. ID No. 1966, Seq. ID No. 1968, Seq. ID No. 1972, Seq. ID No. 1974, Seq. ID No. 1976, Seq. ID No. 1978, Seq. ID No. 1980, Seq. ID No. 1984, Seq. ID No. 1986, Seq. ID No. 1988, Seq. ID No. 1990, Seq. ID No. 1996, Seq. ID No. 1998, Seq. ID No. 2000, Seq. ID No. 2002, Seq. ID No. 2004, Seq. ID No. 2008, Seq. ID No. 2010, Seq. ID No. 2012, Seq. ID No. 2016, Seq. ID No. 2022, Seq. ID No. 2024, Seq. ID No. 2030, Seq. ID No. 2032, Seq. ID No. 2038, Seq. ID No. 2040, Seq. ID No. 2044, Seq. ID No. 2046, Seq. ID No. 2048, Seq. ID No. 2052, Seq. ID No. 2058, Seq. ID No. 2060, Seq. ID No. 2062, Seq. ID No. 2064, Seq. ID No. 2066, Seq. ID No. 2068, Seq. ID No. 2070, Seq. ID No. 2072, Seq. ID No. 2074, Seq. ID No. 2076, Seq. ID No. 2078, Seq. ID No. 2080, Seq. ID No. 2082, Seq. ID No. 2084, Seq. ID No. 2086, Seq. ID No. 2094, Seq. ID No. 2096, Seq. ID No. 2098, Seq. ID No. 2100, Seq. ID No. 2102, Seq. ID No. 2104, Seq. ID No. 2112, Seq. ID No. 2118, Seq. ID No. 2120, Seq. ID No. 2130, Seq. ID No. 2132, Seq. ID No. 2134, Seq. ID No. 2136, Seq. ID No. 2138, Seq. ID No. 2140, Seq. ID No. 2142, Seq. ID No. 2144, Seq. ID No. 2146, Seq. ID No. 2148, Seq. ID No. 2154, Seq. ID No. 2162, Seq. ID No. 2164, Seq. ID No. 2166, Seq. ID No. 2168, Seq. ID No. 2170, Seq. ID No. 2176, Seq. ID No. 2180, Seq. ID No. 2182, Seq. ID No. 2184, Seq. ID No. 2186, Seq. ID No. 2190, Seq. ID No. 2194, Seq. ID No. 2196, Seq. ID No. 2198, Seq. ID No. 2200, Seq. ID No. 2202, Seq. ID No. 2204, Seq. ID No. 2208, Seq. ID No. 2210, Seq. ID No. 2212, Seq. ID No. 2214, Seq. ID No. 2216, Seq. ID No. 2218, Seq. ID No. 2220, Seq. ID No. 2222, Seq. ID No. 2224, Seq. ID No. 2226, Seq. ID No. 2228, Seq. ID No. 2230, Seq. ID No. 2232, Seq. ID No. 2234, Seq. ID No. 2236, Seq. ID No. 2242, Seq. ID No. 2244, Seq. ID No. 2248, Seq. ID No. 2254, Seq. ID No. 2258, Seq. ID No. 2262, Seq. ID No. 2264, Seq. ID No. 2266, Seq. ID No. 2268, Seq. ID No. 2270, Seq. ID No. 2274, Seq. ID No. 2276, Seq. ID No. 2278, Seq. ID No. 2280, Seq. ID No. 2282, Seq. ID No. 2286, Seq. ID No. 2288, Seq. ID No. 2298, Seq. ID No. 2300, Seq. ID No. 2302, Seq. ID No. 2310, Seq. ID No. 2312, Seq. ID No. 2314, Seq. ID No. 2316, Seq. ID No. 2332, Seq. ID No. 2336, Seq. ID No. 2338, Seq. ID No. 2340, Seq. ID No. 2342, Seq. ID No. 2346, Seq. ID No. 2360, Seq. ID No. 2362, Seq. ID No. 2364, Seq. ID No. 2370, Seq. ID No. 2374, Seq. ID No. 2378, Seq. ID No. 2380, Seq. ID No. 2382, Seq. ID No. 2384, Seq. ID No. 2386, Seq. ID No. 2396, Seq. ID No. 2398, Seq. ID No. 2400, Seq. ID No. 2402, Seq. ID No. 2404, Seq. ID No. 2406, Seq. ID No. 2410, Seq. ID No. 2412, Seq. ID No. 2414, Seq. ID No. 2416, Seq. ID No. 2418, Seq. ID No. 2424, Seq. ID No. 2432, Seq. ID No. 2436, Seq. ID No. 2438, Seq. ID No. 2440, Seq. ID No. 2442, Seq. ID No. 2444, Seq. ID No. 2446, Seq. ID No. 2448, Seq. ID No. 2452, Seq. ID No. 2454, Seq. ID No. 2456, Seq. ID No. 2458, Seq. ID No. 2462, Seq. ID No. 2464, Seq. ID No. 2466, Seq. ID No. 2468, Seq. ID No. 2470, Seq. ID No. 2472, Seq. ID No. 2474, Seq. ID No. 2476, Seq. ID No. 2478, Seq. ID No. 2480, Seq. ID No. 2482, Seq. ID No. 2484, Seq. ID No. 2486, Seq. ID No. 2496, Seq. ID No. 2506, Seq. ID No. 2510, Seq. ID No. 2512, Seq. ID No. 2514, Seq. ID No. 2520, Seq. ID No. 2522, Seq. ID No. 2530, Seq. ID No. 2540, Seq. ID No. 2548, Seq. ID No. 2550, Seq. ID No. 2552, Seq. ID No. 2554, Seq. ID No. 2560, Seq. ID No. 2562, Seq. ID No. 2564, Seq. ID No. 2568, Seq. ID No. 2574, Seq. ID No. 2576, Seq. ID No. 2578, Seq. ID No. 2580, Seq. ID No. 2582, Seq. ID No. 2586, Seq. ID No. 2588, Seq. ID No. 2590, Seq. ID No. 2594, Seq. ID No. 2596, Seq. ID No. 2604, Seq. ID No. 2606, Seq. ID No. 2610, Seq. ID No. 2614, Seq. ID No. 2616, Seq. ID No. 2618, Seq. ID No. 2620, Seq. ID No. 2624, Seq. ID No. 2626, Seq. ID No. 2632, Seq. ID No. 2634, Seq. ID No. 2636, Seq. ID No. 2638, Seq. ID No. 2640, Seq. ID No. 2642, Seq. ID No. 2644, Seq. ID No. 2646, Seq. ID No. 2648, Seq. ID No. 2650, Seq. ID No. 2654, Seq. ID No. 2656, Seq. ID No. 2658, Seq. ID No. 2660, Seq. ID No. 2662, Seq. ID No. 2664, Seq. ID No. 2666, Seq. ID No. 2668, Seq. ID No. 2670, Seq. ID No. 2672, Seq. ID No. 2674, Seq. ID No. 2676, Seq. ID No. 2678, Seq. ID No. 2686, Seq. ID No. 2688, Seq. ID No. 2690, Seq. ID No. 2710, Seq. ID No. 2712, Seq. ID No. 2714, Seq. ID No. 2716, Seq. ID No. 2720, Seq. ID No. 2722, Seq. ID No. 2724, Seq. ID No. 2726, Seq. ID No. 2728, Seq. ID No. 2736, Seq. ID No. 2738, Seq. ID No. 2740, Seq. ID No. 2742, Seq. ID No. 2744, Seq. ID No. 2746, Seq. ID No. 2748, Seq. ID No. 2750, Seq. ID No. 2752, Seq. ID No. 2754, Seq. ID No. 2756, Seq. ID No. 2758, Seq. ID No. 2760, Seq. ID No. 2762, Seq. ID No. 2764, Seq. ID No. 2766, Seq. ID No. 2768, Seq. ID No. 2770, Seq. ID No. 2772, Seq. ID No. 2774, Seq. ID No. 2776, Seq. ID No. 2778, Seq. ID No. 2780, Seq. ID No. 2782, Seq. ID No. 2788, Seq. ID No. 2790, Seq. ID No. 2792, Seq. ID No. 2794, Seq. ID No. 2796, Seq. ID No. 2798, Seq. ID No. 2800, Seq. ID No. 2804, Seq. ID No. 2806, Seq. ID No. 2808, Seq. ID No. 2812, Seq. ID No. 2818, Seq. ID No. 2826, Seq. ID No. 2828, Seq. ID No. 2830, Seq. ID No. 2844, Seq. ID No. 2848, Seq. ID No. 2850, Seq. ID No. 2852, Seq. ID No. 2858, Seq. ID No. 2864, Seq. ID No. 2866, Seq. ID No. 2868, Seq. ID No. 2870, Seq. ID No. 2872, Seq. ID No. 2876, Seq. ID No. 2878, Seq. ID No. 2880, Seq. ID No. 2884, Seq. ID No. 2886, Seq. ID No. 2888, Seq. ID No. 2890, Seq. ID No. 2892, Seq. ID No. 2896, Seq. ID No. 2898, Seq. ID No. 2900, Seq. ID No. 2902, Seq. ID No. 2904, Seq. ID No. 2908, Seq. ID No. 2910, Seq. ID No. 2912, Seq. ID No. 2914, Seq. ID No. 2916, Seq. ID No. 2918, Seq. ID No. 2920, Seq. ID No. 2922, Seq. ID No. 2924, Seq. ID No. 2926, Seq. ID No. 2928, Seq. ID No. 2930, Seq. ID No. 2932, Seq. ID No. 2934, Seq. ID No. 2944, Seq. ID No. 2946, Seq. ID No. 2954, Seq. ID No. 2956, Seq. ID No. 2958, Seq. ID No. 2960, Seq. ID No. 2962, Seq. ID No. 2964, Seq. ID No. 2966, Seq. ID No. 2968, Seq. ID No. 2972, Seq. ID No. 2980, Seq. ID No. 2986, Seq. ID No. 2990, Seq. ID No. 2994, Seq. ID No. 3000, Seq. ID No. 3002, Seq. ID No. 3004, Seq. ID No. 3016, Seq. ID No. 3018, Seq. ID No. 3020, Seq. ID No. 3022, Seq. ID No. 3034, Seq. ID No. 3036, Seq. ID No. 3038, Seq. ID No. 3040, Seq. ID No. 3042, Seq. ID No. 3046, Seq. ID No. 3048, Seq. ID No. 3050, Seq. ID No. 3052, Seq. ID No. 3054, Seq. ID No. 3058, Seq. ID No. 3060, Seq. ID No. 3062, Seq. ID No. 3066, Seq. ID No. 3068, Seq. ID No. 3070, Seq. ID No. 3080, Seq. ID No. 3082, Seq. ID No. 3084, Seq. ID No. 3086, Seq. ID No. 3088, Seq. ID No. 3090, Seq. ID No. 3092, Seq. ID No. 3094, Seq. ID No. 3096, Seq. ID No. 3098, Seq. ID No. 3100, Seq. ID No. 3102, Seq. ID No. 3104, Seq. ID No. 3106, Seq. ID No. 3108, Seq. ID No. 3110, Seq. ID No. 3112, Seq. ID No. 3114, Seq. ID No. 3116, Seq. ID No. 3118, Seq. ID No. 3126, Seq. ID No. 3128, Seq. ID No. 3130, Seq. ID No. 3132, Seq. ID No. 3134, Seq. ID No. 3136, Seq. ID No. 3138, Seq. ID No. 3140, Seq. ID No. 3142, Seq. ID No. 3144, Seq. ID No. 3148, Seq. ID No. 3150, Seq. ID No. 3152, Seq. ID No. 3154, Seq. ID No. 3160, Seq. ID No. 3162, Seq. ID No. 3168, Seq. ID No. 3170, Seq. ID No. 3172, Seq. ID No. 3176, Seq. ID No. 3178, Seq. ID No. 3180, Seq. ID No. 3182, Seq. ID No. 3186, Seq. ID No. 3188, Seq. ID No. 3190, Seq. ID No. 3192, Seq. ID No. 3196, Seq. ID No. 3198, Seq. ID No. 3200, Seq. ID No. 3204, Seq. ID No. 3206, Seq. ID No. 3208, Seq. ID No. 3210, Seq. ID No. 3212, Seq. ID No. 3222, Seq. ID No. 3224, Seq. ID No. 3226, Seq. ID No. 3228, Seq. ID No. 3230, Seq. ID No. 3232, Seq. ID No. 3234, Seq. ID No. 3236, Seq. ID No. 3238, Seq. ID No. 3240, Seq. ID No. 3248, Seq. ID No. 3252, Seq. ID No. 3254, Seq. ID No. 3260, Seq. ID No. 3266, Seq. ID No. 3268, Seq. ID No. 3272, Seq. ID No. 3274, Seq. ID No. 3280, Seq. ID No. 3282, Seq. ID No. 3284, Seq. ID No. 3286, Seq. ID No. 3288, Seq. ID No. 3290, Seq. ID No. 3292, Seq. ID No. 3294, Seq. ID No. 3296, Seq. ID No. 3300, Seq. ID No. 3302, Seq. ID No. 3304, Seq. ID No. 3306, Seq. ID No. 3308, Seq. ID No. 3310, Seq. ID No. 3312, Seq. ID No. 3314, Seq. ID No. 3316, Seq. ID No. 3320, Seq. ID No. 3322, Seq. ID No. 3328, Seq. ID No. 3330, Seq. ID No. 3334, Seq. ID No. 3342, Seq. ID No. 3344, Seq. ID No. 3346, Seq. ID No. 3348, Seq. ID No. 3362, Seq. ID No. 3364; Seq. ID No. 3366, Seq. ID No. 3368, Seq. ID No. 3370, Seq. ID No. 3372, Seq. ID No. 3374, Seq. ID No. 3380, Seq. ID No. 3382, Seq. ID No. 3384, Seq. ID No. 3386, Seq. ID No. 3388, Seq. ID No. 3394, Seq. ID No. 3398, Seq. ID No. 3400, Seq. ID No. 3402, Seq. ID No. 3404, Seq. ID No. 3408, Seq. ID No. 3410, Seq. ID No. 3412, Seq. ID No. 3414, Seq. ID No. 3418, Seq. ID No. 3420, Seq. ID No. 3424, Seq. ID No. 3430, Seq. ID No. 3432, Seq. ID No. 3436, Seq. ID No. 3438, Seq. ID No. 3440, Seq. ID No. 3444, Seq. ID No. 3446, Seq. ID No. 3448, Seq. ID No. 3460, Seq. ID No. 3462, Seq. ID No. 3466, Seq. ID No. 3468, Seq. ID No. 3470, Seq. ID No. 3472, Seq. ID No. 3474, Seq. ID No. 3476, Seq. ID No. 3478, Seq. ID No. 3480, Seq. ID No. 3484, Seq. ID No. 3486, Seq. ID No. 3488, Seq. ID No. 3490, Seq. ID No. 3496, Seq. ID No. 3498, Seq. ID No. 3500, Seq. ID No. 3502, Seq. ID No. 3508, Seq. ID No. 3510, Seq. ID No. 3512, Seq. ID No. 3514, Seq. ID No. 3516, Seq. ID No. 3522, Seq. ID No. 3524, Seq. ID No. 3526, Seq. ID No. 3530, Seq. ID No. 3534, Seq. ID No. 3536, Seq. ID No. 3538, Seq. ID No. 3542, Seq. ID No. 3544, Seq. ID No. 3546, Seq. ID No. 3548, Seq. ID No. 3550, Seq. ID No. 3552, Seq. ID No. 3554, Seq. ID No. 3556, Seq. ID No. 3558, Seq. ID No. 3560, Seq. ID No. 3564, Seq. ID No. 3566, Seq. ID No. 3568, Seq. ID No. 3570, Seq. ID No. 3572, Seq. ID No. 3576, Seq. ID No. 3578, Seq. ID No. 3580, Seq. ID No. 3584, Seq. ID No. 3586, Seq. ID No. 3590, Seq. ID No. 3592, Seq. ID No. 3598, Seq. ID No. 3600, Seq. ID No. 3602, Seq. ID No. 3604, Seq. ID No. 3606, Seq. ID No. 3608, Seq. ID No. 3610, Seq. ID No. 3614, Seq. ID No. 3616, Seq. ID No. 3624, Seq. ID No. 3628, Seq. ID No. 3632, Seq. ID No. 3634, Seq. ID No. 3638, Seq. ID No. 3640, Seq. ID No. 3642, Seq. ID No. 3644, Seq. ID No. 3646, Seq. ID No. 3648, Seq. ID No. 3650, Seq. ID No. 3652, Seq. ID No. 3654, Seq. ID No. 3656, Seq. ID No. 3658, Seq. ID No. 3660, Seq. ID No. 3662, Seq. ID No. 3664, Seq. ID No. 3666, Seq. ID No. 3668, Seq. ID No. 3670, Seq. ID No. 3672, Seq. ID No. 3674, Seq. ID No. 3676, Seq. ID No. 3682, Seq. ID No. 3684, Seq. ID No. 3686, Seq. ID No. 3688, Seq. ID No. 3692, Seq. ID No. 3694, Seq. ID No. 3696, Seq. ID No. 3698, Seq. ID No. 3700, Seq. ID No. 3702, Seq. ID No. 3704, Seq. ID No. 3706, Seq. ID No. 3708, Seq. ID No. 3710, Seq. ID No. 3712, Seq. ID No. 3714, Seq. ID No. 3716, Seq. ID No. 3720, Seq. ID No. 3722, Seq. ID No. 3726, Seq. ID No. 3730, Seq. ID No. 3732, Seq. ID No. 3734, Seq. ID No. 3736, Seq. ID No. 3738, Seq. ID No. 3740, Seq. ID No. 3744, Seq. ID No. 3748, Seq. ID No. 3750, Seq. ID No. 3752, Seq. ID No. 3760, Seq. ID No. 3768, Seq. ID No. 3770, Seq. ID No. 3772, Seq. ID No. 3774, Seq. ID No. 3776, Seq. ID No. 3778, Seq. ID No. 3780, Seq. ID No. 3786, Seq. ID No. 3788, Seq. ID No. 3790, Seq. ID No. 3792, Seq. ID No. 3796, Seq. ID No. 3798, Seq. ID No. 3802, Seq. ID No. 3804, Seq. ID No. 3806, Seq. ID No. 3810, Seq. ID No. 3812, Seq. ID No. 3820, Seq. ID No. 3822, Seq. ID No. 3824, Seq. ID No. 3826, Seq. ID No. 3828, Seq. ID No. 3830, Seq. ID No. 3832, Seq. ID No. 3834, Seq. ID No. 3836, Seq. ID No. 3842, Seq. ID No. 3844, Seq. ID No. 3846, Seq. ID No. 3848, Seq. ID No. 3856, Seq. ID No. 3858, Seq. ID No. 3868, Seq. ID No. 3870, Seq. ID No. 3872, Seq. ID No. 3874, Seq. ID No. 3876, Seq. ID No. 3878, Seq. ID No. 3882, Seq. ID No. 3884, Seq. ID No. 3886, Seq. ID No. 3888, Seq. ID No. 3894, Seq. ID No. 3898, Seq. ID No. 3900, Seq. ID No. 3902, Seq. ID No. 3906, Seq. ID No. 3908, Seq. ID No. 3910, Seq. ID No. 3912, Seq. ID No. 3914, Seq. ID No. 3918, Seq. ID No. 3920, Seq. ID No. 3922, Seq. ID No. 3928, Seq. ID No. 3930, Seq. ID No. 3932, Seq. ID No. 3934, Seq. ID No. 3936, Seq. ID No. 3938, Seq. ID No. 3940, Seq. ID No. 3942, Seq. ID No. 3944, Seq. ID No. 3946, Seq. ID No. 3948, Seq. ID No. 3950, Seq. ID No. 3952, Seq. ID No. 3954, Seq. ID No. 3956, Seq. ID No. 3958, Seq. ID No. 3962, Seq. ID No. 3966, Seq. ID No. 3968, Seq. ID No. 3970, Seq. ID No. 3972, Seq. ID No. 3978, Seq. ID No. 3980, Seq. ID No. 3982, Seq. ID No. 3984, Seq. ID No. 3986, Seq. ID No. 3988, Seq. ID No. 3990, Seq. ID No. 3992, Seq. ID No. 4000, Seq. ID No. 4002, Seq. ID No. 4004, Seq. ID No. 4012, Seq. ID No. 4014, Seq. ID No. 4016, Seq. ID No. 4018, Seq. ID No. 4020, Seq. ID No. 4022, Seq. ID No. 4024, Seq. ID No. 4030, Seq. ID No. 4032, Seq. ID No. 4034, Seq. ID No. 4036, Seq. ID No. 4038, Seq. ID No. 4052, Seq. ID No. 4054, Seq. ID No. 4056, Seq. ID No. 4058, Seq. ID No. 4060, Seq. ID No. 4064, Seq. ID No. 4066, Seq. ID No. 4068, Seq. ID No. 4070, Seq. ID No. 4076, Seq. ID No. 4078, Seq. ID No. 4086, Seq. ID No. 4088, Seq. ID No. 4090, Seq. ID No. 4092, Seq. ID No. 4094, Seq. ID No. 4096, Seq. ID No. 4098, Seq. ID No. 4100, Seq. ID No. 4106, Seq. ID No. 4108, Seq. ID No. 4110, Seq. ID No. 4114, Seq. ID No. 4116, Seq. ID No. 4118, Seq. ID No. 4120, Seq. ID No. 4122, Seq. ID No. 4124, Seq. ID No. 4126, Seq. ID No. 4128, Seq. ID No. 4130, Seq. ID No. 4132, Seq. ID No. 4148, Seq. ID No. 4150, Seq. ID No. 4156, Seq. ID No. 4158, Seq. ID No. 4160, Seq. ID No. 4162, Seq. ID No. 4164, Seq. ID No. 4166, Seq. ID No. 4168, Seq. ID No. 4170, Seq. ID No. 4172, Seq. ID No. 4174, Seq. ID No. 4176, Seq. ID No. 4178, Seq. ID No. 4180, Seq. ID No. 4184, Seq. ID No. 4186, Seq. ID No. 4188, Seq. ID No. 4190, Seq. ID No. 4192, Seq. ID No. 4194, Seq. ID No. 4196, Seq. ID No. 4198, Seq. ID No. 4200, Seq. ID No. 4202, Seq. ID No. 4204, Seq. ID No. 4206, Seq. ID No. 4208, Seq. ID No. 4210, Seq. ID No. 4212, Seq. ID No. 4214, Seq. ID No. 4216, Seq. ID No. 4218, Seq. ID No. 4220, Seq. ID No. 4230, Seq. ID No. 4236, Seq. ID No. 4238, Seq. ID No. 4240, Seq. ID No. 4244, Seq. ID No. 4246, Seq. ID No. 4250, Seq. ID No. 4252, Seq. ID No. 4256, Seq. ID No. 4268, Seq. ID No. 4272, Seq. ID No. 4274, Seq. ID No. 4278, Seq. ID No. 4280, Seq. ID No. 4282, Seq. ID No. 4284, Seq. ID No. 4290, Seq. ID No. 4292, Seq. ID No. 4294, Seq. ID No. 4296, Seq. ID No. 4300, Seq. ID No. 4302, Seq. ID No. 4304, Seq. ID No. 4306, Seq. ID No. 4310, Seq. ID No. 4316, Seq. ID No. 4318, Seq. ID No. 4320, Seq. ID No. 4322, Seq. ID No. 4324, Seq. ID No. 4326, Seq. ID No. 4328, Seq. ID No. 4330, Seq. ID No. 4332, Seq. ID No. 4334, Seq. ID No. 4336, Seq. ID No. 4338, Seq. ID No. 4340, Seq. ID No. 4342, Seq. ID No. 4346, Seq. ID No. 4348, Seq. ID No. 4350, Seq. ID No. 4352, Seq. ID No. 4354, Seq. ID No. 4356, Seq. ID No. 4358, Seq. ID No. 4360, Seq. ID No. 4362, Seq. ID No. 4364, Seq. ID No. 4366, Seq. ID No. 4368, Seq. ID No. 4370, Seq. ID No. 4372, Seq. ID No. 4374, Seq. ID No. 4376, Seq. ID No. 4378, Seq. ID No. 4382, Seq. ID No. 4384, Seq. ID No. 4388, Seq. ID No. 4394, Seq. ID No. 4396, Seq. ID No. 4398, Seq. ID No. 4402, Seq. ID No. 4406, Seq. ID No. 4408, Seq. ID No. 4410, Seq. ID No. 4412, Seq. ID No. 4414, Seq. ID No. 4416, Seq. ID No. 4418, Seq. ID No. 4420, Seq. ID No. 4422, Seq. ID No. 4424, Seq. ID No. 4426, Seq. ID No. 4428, Seq. ID No. 4430, Seq. ID No. 4432, Seq. ID No. 4434, Seq. ID No. 4436, Seq. ID No. 4444, Seq. ID No. 4446, Seq. ID No. 4448, Seq. ID No. 4450, Seq. ID No. 4452, Seq. ID No. 4454, Seq. ID No. 4456, Seq. ID No. 4458, Seq. ID No. 4460, Seq. ID No. 4462, Seq. ID No. 4464, Seq. ID No. 4466, Seq. ID No. 4468, Seq. ID No. 4472, Seq. ID No. 4476, Seq. ID No. 4478, Seq. ID No. 4480, Seq. ID No. 4488, Seq. ID No. 4490, Seq. ID No. 4492, Seq. ID No. 4500, Seq. ID No. 4502, Seq. ID No. 4504, Seq. ID No. 4506, Seq. ID No. 4508, Seq. ID No. 4510, Seq. ID No. 4512, Seq. ID No. 4518, Seq. ID No. 4520, Seq. ID No. 4522, Seq. ID No. 4524, Seq. ID No. 4526, Seq. ID No. 4532, Seq. ID No. 4536, Seq. ID No. 4538, Seq. ID No. 4540, Seq. ID No. 4542, Seq. ID No. 4544, Seq. ID No. 4546, Seq. ID No. 4548, Seq. ID No. 4550, Seq. ID No. 4552, Seq. ID No. 4554, Seq. ID No. 4556, Seq. ID No. 4558, Seq. ID No. 4560, Seq. ID No. 4562, Seq. ID No. 4564, Seq. ID No. 4566, Seq. ID No. 4568, Seq. ID No. 4570, Seq. ID No. 4572, Seq. ID No. 4574, Seq. ID No. 4576, Seq. ID No. 4578, Seq. ID No. 4580, Seq. ID No. 4582, Seq. ID No. 4584, Seq. ID No. 4586, Seq. ID No. 4592, Seq. ID No. 4594, Seq. ID No. 4596, Seq. ID No. 4598, Seq. ID No. 4600, Seq. ID No. 4602, Seq. ID No. 4604, Seq. ID No. 4606, Seq. ID No. 4608, Seq. ID No. 4610, Seq. ID No. 4612, Seq. ID No. 4614, Seq. ID No. 4616, Seq. ID No. 4618, Seq. ID No. 4620, Seq. ID No. 4622, Seq. ID No. 4624, Seq. ID No. 4626, Seq. ID No. 4628, Seq. ID No. 4630, Seq. ID No. 4632, Seq. ID No. 4634, Seq. ID No. 4636, Seq. ID No. 4638, Seq. ID No. 4646, Seq. ID No. 4648, Seq. ID No. 4650, Seq. ID No. 4652, Seq. ID No. 4654, Seq. ID No. 4656, Seq. ID No. 4658, Seq. ID No. 4662, Seq. ID No. 4664, Seq. ID No. 4666, Seq. ID No. 4668, Seq. ID No. 4670, Seq. ID No. 4672, Seq. ID No. 4674, Seq. ID No. 4676, Seq. ID No. 4678, Seq. ID No. 4680, Seq. ID No. 4684, Seq. ID No. 4686, Seq. ID No. 4690, Seq. ID No. 4692, Seq. ID No. 4694, Seq. ID No. 4698, Seq. ID No. 4700, Seq. ID No. 4702, Seq. ID No. 4704, Seq. ID No. 4714, Seq. ID No. 4720, Seq. ID No. 4722, Seq. ID No. 4724, Seq. ID No. 4726, Seq. ID No. 4728, Seq. ID No. 4730, Seq. ID No. 4732, Seq. ID No. 4734, Seq. ID No. 4736, Seq. ID No. 4740, Seq. ID No. 4742, Seq. ID No. 4744, Seq. ID No. 4746, Seq. ID No. 4748, Seq. ID No. 4750, Seq. ID No. 4752, Seq. ID No. 4754, Seq. ID No. 4756, Seq. ID No. 4758, Seq. ID No. 4762, Seq. ID No. 4764, Seq. ID No. 4766, Seq. ID No. 4768, Seq. ID No. 4770, Seq. ID No. 4772, Seq. ID No. 4774, Seq. ID No. 4776, Seq. ID No. 4778, Seq. ID No. 4780, Seq. ID No. 4782, Seq. ID No. 4784, Seq. ID No. 4786, Seq. ID No. 4788, Seq. ID No. 4790, Seq. ID No. 4792, Seq. ID No. 4794, Seq. ID No. 4796, Seq. ID No. 4798, Seq. ID No. 4800, Seq. ID No. 4802, Seq. ID No. 4804, Seq. ID No. 4806, Seq. ID No. 4808, Seq. ID No. 4810, Seq. ID No. 4812, Seq. ID No. 4816, Seq. ID No. 4818, Seq. ID No. 4820, Seq. ID No. 4822, Seq. ID No. 4824, Seq. ID No. 4826, Seq. ID No. 4830, Seq. ID No. 4832, Seq. ID No. 4834, Seq. ID No. 4836, Seq. ID No. 4838, Seq. ID No. 4840, Seq. ID No. 4842, Seq. ID No. 4844, Seq. ID No. 4846, Seq. ID No. 4848, Seq. ID No. 4850, Seq. ID No. 4852, Seq. ID No. 4856, Seq. ID No. 4858, Seq. ID No. 4860, Seq. ID No. 4862, Seq. ID No. 4866, Seq. ID No. 4868, Seq. ID No. 4878, Seq. ID No. 4880, Seq. ID No. 4882, Seq. ID No. 4884, Seq. ID No. 4886, Seq. ID No. 4888, Seq. ID No. 4890, Seq. ID No. 4892, Seq. ID No. 4894, Seq. ID No. 4896, Seq. ID No. 4898, Seq. ID No. 4900, Seq. ID No. 4902, Seq. ID No. 4904, Seq. ID No. 4906, Seq. ID No. 4908, Seq. ID No. 4910, Seq. ID No. 4912, Seq. ID No. 4914, Seq. ID No. 4916, Seq. ID No. 4918, Seq. ID No. 4926, Seq. ID No. 4928, Seq. ID No. 4934, Seq. ID No. 4936, Seq. ID No. 4938, Seq. ID No. 4940, Seq. ID No. 4942, Seq. ID No. 4944, Seq. ID No. 4946, Seq. ID No. 4948, Seq. ID No. 4954, Seq. ID No. 4956, Seq. ID No. 4958, Seq. ID No. 4960, Seq. ID No. 4962, Seq. ID No. 4964, Seq. ID No. 4966, Seq. ID No. 4968, Seq. ID No. 4970, Seq. ID No. 4972, Seq. ID No. 4974, Seq. ID No. 4978, Seq. ID No. 4980, Seq. ID No. 4982, Seq. ID No. 4984, Seq. ID No. 4986, Seq. ID No. 4990, Seq. ID No. 4992, Seq. ID No. 4994, Seq. ID No. 4996, Seq. ID No. 4998, Seq. ID No. 5002, Seq. ID No. 5004, Seq. ID No. 5006, Seq. ID No. 5008, Seq. ID No. 5010, Seq. ID No. 5012, Seq. ID No. 5016, Seq. ID No. 5018, Seq. ID No. 5020, Seq. ID No. 5022, Seq. ID No. 5024, Seq. ID No. 5026, Seq. ID No. 5028, Seq. ID No. 5030, Seq. ID No. 5034, Seq. ID No. 5036, Seq. ID No. 5038, Seq. ID No. 5040, Seq. ID No. 5042, Seq. ID No. 5044, Seq. ID No. 5046, Seq. ID No. 5048, Seq. ID No. 5050, Seq. ID No. 5052, Seq. ID No. 5054, Seq. ID No. 5056, Seq. ID No. 5058, Seq. ID No. 5060, Seq. ID No. 5066, Seq. ID No. 5068, Seq. ID No. 5072, Seq. ID No. 5074, Seq. ID No. 5076, Seq. ID No. 5078, Seq. ID No. 5080, Seq. ID No. 5082, Seq. ID No. 5084, Seq. ID No. 5088, Seq. ID No. 5090, Seq. ID No. 5092, Seq. ID No. 5094, Seq. ID No. 5096, Seq. ID No. 5098, Seq. ID No. 5100, Seq. ID No. 5102, Seq. ID No. 5106, Seq. ID No. 5108, Seq. ID No. 5110, Seq. ID No. 5112, Seq. ID No. 5114, Seq. ID No. 5118, Seq. ID No. 5120, Seq. ID No. 5122, Seq. ID No. 5128, Seq. ID No. 5144, Seq. ID No. 5146, Seq. ID No. 5148, Seq. ID No. 5150, Seq. ID No. 5152, Seq. ID No. 5154, Seq. ID No. 5156, Seq. ID No. 5158, Seq. ID No. 5162, Seq. ID No. 5164, Seq. ID No. 5168, Seq. ID No. 5170, Seq. ID No. 5172, Seq. ID No. 5174, Seq. ID No. 5178, Seq. ID No. 5180, Seq. ID No. 5182, Seq. ID No. 5184, Seq. ID No. 5186, Seq. ID No. 5188, Seq. ID No. 5190, Seq. ID No. 5192, Seq. ID No. 5194, Seq. ID No. 5196, Seq. ID No. 5202, Seq. ID No. 5204, Seq. ID No. 5210, Seq. ID No. 5212, Seq. ID No. 5216, Seq. ID No. 5218, Seq. ID No. 5222, Seq. ID No. 5226, Seq. ID No. 5228, Seq. ID No. 5230, Seq. ID No. 5232, Seq. ID No. 5234, Seq. ID No. 5236, Seq. ID No. 5238, Seq. ID No. 5240, Seq. ID No. 5242, Seq. ID No. 5244, Seq. ID No. 5246, Seq. ID No. 5248, Seq. ID No. 5250, Seq. ID No. 5252, Seq. ID No. 5254, Seq. ID No. 5256, Seq. ID No. 5258, Seq. ID No. 5260, Seq. ID No. 5262, Seq. ID No. 5264, Seq. ID No. 5266, Seq. ID No. 5270, Seq. ID No. 5272, Seq. ID No. 5284, Seq. ID No. 5288, Seq. ID No. 5290, Seq. ID No. 5292, Seq. ID No. 5294, Seq. ID No. 5296, Seq. ID No. 5298, Seq. ID No. 5300, Seq. ID No. 5302, Seq. ID No. 5304, Seq. ID No. 5306, Seq. ID No. 5308, Seq. ID No. 5310, Seq. ID No. 5312, Seq. ID No. 5314, Seq. ID No. 5316, Seq. ID No. 5318, Seq. ID No. 5320, Seq. ID No. 5322, Seq. ID No. 5324, Seq. ID No. 5326, Seq. ID No. 5334, Seq. ID No. 5336, Seq. ID No. 5338, Seq. ID No. 5340, Seq. ID No. 5344, Seq. ID No. 5346, Seq. ID No. 5348, Seq. ID No. 5350, Seq. ID No. 5352, Seq. ID No. 5354, Seq. ID No. 5356, Seq. ID No. 5358, Seq. ID No. 5360, Seq. ID No. 5362, Seq. ID No. 5364, Seq. ID No. 5366, Seq. ID No. 5368, Seq. ID No. 5370, Seq. ID No. 5372, Seq. ID No. 5374, Seq. ID No. 5376, Seq. ID No. 5382, Seq. ID No. 5384, Seq. ID No. 5390, Seq. ID No. 5392, Seq. ID No. 5394, Seq. ID No. 5396, Seq. ID No. 5398, Seq. ID No. 5400, Seq. ID No. 5402, Seq. ID No. 5404, Seq. ID No. 5408, Seq. ID No. 5410, Seq. ID No. 5412, Seq. ID No. 5418, Seq. ID No. 5420, Seq. ID No. 5422, Seq. ID No. 5424, Seq. ID No. 5426, Seq. ID No. 5428, Seq. ID No. 5430, Seq. ID No. 5432, Seq. ID No. 5436, Seq. ID No. 5438, Seq. ID No. 5440, Seq. ID No. 5442, Seq. ID No. 5444, Seq. ID No. 5446, Seq. ID No. 5448, Seq. ID No. 5450, Seq. ID No. 5452, Seq. ID No. 5456, Seq. ID No. 5458, Seq. ID No. 5468, Seq. ID No. 5470, Seq. ID No. 5472, Seq. ID No. 5474, Seq. ID No. 5476, Seq. ID No. 5478, Seq. ID No. 5480, Seq. ID No. 5482, Seq. ID No. 5484, Seq. ID No. 5486, Seq. ID No. 5488, Seq. ID No. 5490, Seq. ID No. 5494, Seq. ID No. 5496, Seq. ID No. 5500, Seq. ID No. 5502, Seq. ID No. 5504, Seq. ID No. 5506, Seq. ID No. 5508, Seq. ID No. 5512, Seq. ID No. 5520, Seq. ID No. 5522, Seq. ID No. 5524, Seq. ID No. 5528, Seq. ID No. 5536, Seq. ID No. 5538, Seq. ID No. 5540, Seq. ID No. 5542, Seq. ID No. 5548, Seq. ID No. 5550, Seq. ID No. 5552, Seq. ID No. 5554, Seq. ID No. 5556, Seq. ID No. 5558, Seq. ID No. 5560, Seq. ID No. 5562, Seq. ID No. 5564, Seq. ID No. 5566, Seq. ID No. 5568, Seq. ID No. 5572, Seq. ID No. 5574, Seq. ID No. 5576, Seq. ID No. 5578, Seq. ID No. 5580, Seq. ID No. 5582, Seq. ID No. 5584, Seq. ID No. 5586, Seq. ID No. 5600, Seq. ID No. 5602, Seq. ID No. 5604, Seq. ID No. 5606, Seq. ID No. 5608, Seq. ID No. 5614, Seq. ID No. 5616, Seq. ID No. 5618, Seq. ID No. 5620, Seq. ID No. 5628, Seq. ID No. 5630, Seq. ID No. 5632, Seq. ID No. 5636, Seq. ID No. 5646, Seq. ID No. 5648, Seq. ID No. 5652, Seq. ID No. 5654, Seq. ID No. 5656, Seq. ID No. 5658, Seq. ID No. 5662, Seq. ID No. 5664, Seq. ID No. 5666, Seq. ID No. 5670, Seq. ID No. 5672, Seq. ID No. 5674, Seq. ID No. 5676, Seq. ID No. 5678, Seq. ID No. 5680, Seq. ID No. 5692, Seq. ID No. 5694, Seq. ID No. 5696, Seq. ID No. 5698, Seq. ID No. 5702, Seq. ID No. 5708, Seq. ID No. 5714, Seq. ID No. 5716, Seq. ID No. 5718, Seq. ID No. 5722, Seq. ID No. 5724, Seq. ID No. 5726, Seq. ID No. 5728, Seq. ID No. 5732, Seq. ID No. 5738, Seq. ID No. 5742, Seq. ID No. 5744, Seq. ID No. 5746, Seq. ID No. 5748, Seq. ID No. 5750, Seq. ID No. 5752, Seq. ID No. 5754, Seq. ID No. 5756, Seq. ID No. 5758, Seq. ID No. 5760, Seq. ID No. 5762, Seq. ID No. 5770, Seq. ID No. 5774, Seq. ID No. 5776, Seq. ID No. 5778, Seq. ID No. 5780, Seq. ID No. 5782, Seq. ID No. 5788, Seq. ID No. 5790, Seq. ID No. 5794, Seq. ID No. 5796, Seq. ID No. 5800, Seq. ID No. 5802, Seq. ID No. 5804, Seq. ID No. 5806, Seq. ID No. 5808, Seq. ID No. 5810, Seq. ID No. 5812, Seq. ID No. 5814, Seq. ID No. 5816, Seq. ID No. 5818, Seq. ID No. 5820, Seq. ID No. 5822, Seq. ID No. 5824, Seq. ID No. 5826, Seq. ID No. 5828, Seq. ID No. 5832, Seq. ID No. 5834, Seq. ID No. 5836, Seq. ID No. 5838, Seq. ID No. 5840, Seq. ID No. 5842, Seq. ID No. 5844, Seq. ID No. 5846, Seq. ID No. 5848, Seq. ID No. 5850, Seq. ID No. 5852, Seq. ID No. 5854, Seq. ID No. 5856, Seq. ID No. 5858, Seq. ID No. 5860, Seq. ID No. 5862, Seq. ID No. 5864, Seq. ID No. 5866, Seq. ID No. 5868, Seq. ID No. 5870, Seq. ID No. 5872, Seq. ID No. 5874, Seq. ID No. 5876, Seq. ID No. 5878, Seq. ID No. 5882, Seq. ID No. 5888, Seq. ID No. 5892, Seq. ID No. 5898, Seq. ID No. 5904, Seq. ID No. 5908, Seq. ID No. 5912, Seq. ID No. 5914, Seq. ID No. 5916, Seq. ID No. 5918, Seq. ID No. 5920, Seq. ID No. 5926, Seq. ID No. 5932, Seq. ID No. 5934, Seq. ID No. 5936, Seq. ID No. 5938, Seq. ID No. 5942, Seq. ID No. 5944, Seq. ID No. 5946, Seq. ID No. 5948, Seq. ID No. 5950, Seq. ID No. 5952, Seq. ID No. 5954, Seq. ID No. 5956, Seq. ID No. 5958, Seq. ID No. 5960, Seq. ID No. 5962, Seq. ID No. 5964, Seq. ID No. 5966, Seq. ID No. 5968, Seq. ID No. 5970, Seq. ID No. 5972, Seq. ID No. 5974, Seq. ID No. 5976, Seq. ID No. 5978, Seq. ID No. 5980, Seq. ID No. 5982, Seq. ID No. 5984, Seq. ID No. 5986, Seq. ID No. 5988, Seq. ID No. 5990, Seq. ID No. 5992, Seq. ID No. 5994, Seq. ID No. 5996, Seq. ID No. 5998, Seq. ID No. 6000, Seq. ID No. 6002, Seq. ID No. 6004, Seq. ID No. 6006, Seq. ID No. 6008, Seq. ID No. 6010, Seq. ID No. 6012, Seq. ID No. 6014, Seq. ID No. 6016, Seq. ID No. 6018, Seq. ID No. 6020, Seq. ID No. 6022, Seq. ID No. 6030, Seq. ID No. 6032, Seq. ID No. 6034, Seq. ID No. 6036, Seq. ID No. 6038, Seq. ID No. 6040, Seq. ID No. 6042, Seq. ID No. 6046, Seq. ID No. 6050, Seq. ID No. 6052, Seq. ID No. 6056, Seq. ID No. 6058, Seq. ID No. 6062, Seq. ID No. 6066, Seq. ID No. 6070, Seq. ID No. 6072, Seq. ID No. 6074, Seq. ID No. 6076, Seq. ID No. 6078, Seq. ID No. 6080, Seq. ID No. 6082, Seq. ID No. 6084, Seq. ID No. 6086, Seq. ID No. 6094, Seq. ID No. 6098, Seq. ID No. 6100, Seq. ID No. 6102, Seq. ID No. 6106, Seq. ID No. 6114, Seq. ID No. 6120, Seq. ID No. 6122, Seq. ID No. 6124, Seq. ID No. 6126, Seq. ID No. 6128, Seq. ID No. 6130, Seq. ID No. 6132, Seq. ID No. 6134, Seq. ID No. 6140, Seq. ID No. 6142, Seq. ID No. 6144, Seq. ID No. 6146, Seq. ID No. 6148, Seq. ID No. 6150, Seq. ID No. 6152, Seq. ID No. 6154, Seq. ID No. 6156, Seq. ID No. 6158, Seq. ID No. 6168, Seq. ID No. 6172, Seq. ID No. 6174, Seq. ID No. 6176, Seq. ID No. 6178, Seq. ID No. 6180, Seq. ID No. 6182, Seq. ID No. 6194, Seq. ID No. 6196, Seq. ID No. 6200, Seq. ID No. 6202, Seq. ID No. 6208, Seq. ID No. 6212, Seq. ID No. 6214, Seq. ID No. 6216, Seq. ID No. 6220, Seq. ID No. 6232, Seq. ID No. 6234, Seq. ID No. 6240, Seq. ID No. 6248, Seq. ID No. 6252, Seq. ID No. 6256, Seq. ID No. 6258, Seq. ID No. 6262, Seq. ID No. 6270, Seq. ID No. 6272, Seq. ID No. 6278, Seq. ID No. 6280, Seq. ID No. 6282, Seq. ID No. 6288, Seq. ID No. 6292, Seq. ID No. 6294, Seq. ID No. 6296, Seq. ID No. 6298, Seq. ID No. 6300, Seq. ID No. 6306, Seq. ID No. 6308, Seq. ID No. 6310, Seq. ID No. 6312, Seq. ID No. 6314, Seq. ID No. 6316, Seq. ID No. 6313, Seq. ID No. 6320, Seq. ID No. 6322, Seq. ID No. 6324, Seq. ID No. 6330, Seq. ID No. 6332, Seq. ID No. 6334, Seq. ID No. 6336, Seq. ID No. 6338, Seq. ID No. 6340, Seq. ID No. 6342, Seq. ID No. 6344, Seq. ID No. 6346, Seq. ID No. 6348, Seq. ID No. 6350, Seq. ID No. 6352, Seq. ID No. 6354, Seq. ID No. 6356, Seq. ID No. 6358, Seq. ID No. 6360, Seq. ID No. 6362, Seq. ID No. 6368, Seq. ID No. 6370, Seq. ID No. 6372, Seq. ID No. 6374, Seq. ID No. 6376, Seq. ID No. 6380, Seq. ID No. 6386, Seq. ID No. 6388, Seq. ID No. 6390, Seq. ID No. 6392, Seq. ID No. 6394, Seq. ID No. 6396, Seq. ID No. 6398, Seq. ID No. 6400, Seq. ID No. 6402, Seq. ID No. 6404, Seq. ID No. 6406, Seq. ID No. 6408, Seq. ID No. 6410, Seq. ID No. 6412, Seq. ID No. 6414, Seq. ID No. 6418, Seq. ID No. 6420, Seq. ID No. 6422, Seq. ID No. 6424, Seq. ID No. 6434, Seq. ID No. 6436, Seq. ID No. 6438, Seq. ID No. 6440, Seq. ID No. 6442, Seq. ID No. 6444, Seq. ID No. 6446, Seq. ID No. 6448, Seq. ID No. 6450, Seq. ID No. 6452, Seq. ID No. 6454, Seq. ID No. 6456, Seq. ID No. 6458, Seq. ID No. 6460, Seq. ID No. 6462, Seq. ID No. 6464, Seq. ID No. 6466, Seq. ID No. 6468, Seq. ID No. 6470, Seq. ID No. 6472, Seq. ID No. 6476, Seq. ID No. 6478, Seq. ID No. 6482, Seq. ID No. 6484, Seq. ID No. 6486, Seq. ID No. 6488, Seq. ID No. 6490, Seq. ID No. 6492, Seq. ID No. 6494, Seq. ID No. 6496, Seq. ID No. 6500, Seq. ID No. 6502, Seq. ID No. 6504, Seq. ID No. 6506, Seq. ID No. 6508, Seq. ID No. 6510, Seq. ID No. 6512, Seq. ID No. 6514, Seq. ID No. 6516, Seq. ID No. 6520, Seq. ID No. 6522, Seq. ID No. 6524, Seq. ID No. 6526, Seq. ID No. 6528, Seq. ID No. 6530, Seq. ID No. 6532, Seq. ID No. 6534, Seq. ID No. 6536, Seq. ID No. 6538, Seq. ID No. 6540, Seq. ID No. 6546, Seq. ID No. 6548, Seq. ID No. 6550, Seq. ID No. 6552, Seq. ID No. 6554, Seq. ID No. 6560, Seq. ID No. 6562, Seq. ID No. 6564, Seq. ID No. 6566, Seq. ID No. 6568, Seq. ID No. 6570, Seq. ID No. 6576, Seq. ID No. 6578, Seq. ID No. 6580, Seq. ID No. 6584, Seq. ID No. 6586, Seq. ID No. 6588, Seq. ID No. 6590, Seq. ID No. 6592, Seq. ID No. 6606, Seq. ID No. 6608, Seq. ID No. 6610, Seq. ID No. 6612, Seq. ID No. 6614, Seq. ID No. 6616, Seq. ID No. 6618, Seq. ID No. 6620, Seq. ID No. 6622, Seq. ID No. 6624, Seq. ID No. 6626, Seq. ID No. 6628, Seq. ID No. 6630, Seq. ID No. 6634, Seq. ID No. 6636, Seq. ID No. 6642, and Seq. ID No. 6648.

In another preferred embodiment, the polypeptide is selected from the Alloiococcus otitidis polypeptides identified by BlastP analysis and is selected from one of Seq. ID No. 10, Seq. ID No. 18, Seq. ID No. 46, Seq. ID No. 50, Seq. ID No. 54, Seq. ID No. 60, Seq. ID No. 90, Seq. ID No. 146, Seq. ID No. 158, Seq. ID No. 168, Seq. ID No. 172, Seq. ID No. 192, Seq. ID No. 216, Seq. ID No. 218, Seq. ID No. 226, Seq. ID No. 240, Seq. ID No. 254, Seq. ID No. 274, Seq. ID No. 278, Seq. ID No. 290, Seq. ID No. 330, Seq. ID No. 344, Seq. ID No. 346, Seq. ID No. 376, Seq. ID No. 502, Seq. ID No. 506, Seq. ID No. 512, Seq. ID No. 516, Seq. ID No. 530, Seq. ID No. 570, Seq. ID No. 576, Seq. ID No. 594, Seq. ID No. 596, Seq. ID No. 638, Seq. ID No. 660, Seq. ID No. 670, Seq. ID No. 676, Seq. ID No. 682, Seq. ID No. 730, Seq. ID No. 780, Seq. ID No. 820, Seq. ID No. 896, Seq. ID No. 922, Seq. ID No. 936, Seq. ID No. 956, Seq. ID No. 968, Seq. ID No. 986, Seq. ID No. 1018, Seq. ID No. 1040, Seq. ID No. 1076, Seq. ID No. 1090, Seq. ID No. 1138, Seq. ID No. 1224, Seq. ID No. 1242, Seq. ID No. 1316, Seq. ID No. 1332, Seq. ID No. 1370, Seq. ID No. 1460, Seq. ID No. 1518, Seq. ID No. 1562, Seq. ID No. 1564, Seq. ID No. 1568, Seq. ID No. 1572, Seq. ID No. 1574, Seq. ID No. 1602, Seq. ID No. 1608, Seq. ID No. 1614, Seq. ID No. 1620, Seq. ID No. 1642, Seq. ID No. 1648, Seq. ID No. 1654, Seq. ID No. 1658, Seq. ID No. 1666, Seq. ID No. 1700, Seq. ID No. 1714, Seq. ID No. 1752, Seq. ID No. 1772, Seq. ID No. 1786, Seq. ID No. 1790, Seq. ID No. 1792, Seq. ID No. 1798, Seq. ID No. 1812, Seq. ID No. 1818, Seq. ID No. 1820, Seq. ID No. 1822, Seq. ID No. 1872, Seq. ID No. 1896, Seq. ID No. 1906, Seq. ID No. 1914, Seq. ID No. 1920, Seq. ID No. 1924, Seq. ID No. 1966, Seq. ID No. 1972, Seq. ID No. 2000, Seq. ID No. 2048, Seq. ID No. 2056, Seq. ID No. 2092, Seq. ID No. 2094, Seq. ID No. 2116, Seq. ID No. 2120, Seq. ID No. 2128, Seq. ID No. 2166, Seq. ID No. 2180, Seq. ID No. 2208, Seq. ID No. 2224, Seq. ID No. 2228, Seq. ID No. 2242, Seq. ID No. 2254, Seq. ID No. 2264, Seq. ID No. 2270, Seq. ID No. 2276, Seq. ID No. 2302, Seq. ID No. 2316, Seq. ID No. 2330, Seq. ID No. 2334, Seq. ID No. 2344, Seq. ID No. 2346, Seq. ID No. 2364, Seq. ID No. 2374, Seq. ID No. 2392, Seq. ID No. 2418, Seq. ID No. 2430, Seq. ID No. 2466, Seq. ID No. 2476, Seq. ID No. 2484, Seq. ID No. 2514, Seq. ID No. 2526, Seq. ID No. 2590, Seq. ID No. 2614, Seq. ID No. 2652, Seq. ID No. 2670, Seq. ID No. 2678, Seq. ID No. 2688, Seq. ID No. 2690, Seq. ID No. 2694, Seq. ID No. 2704, Seq. ID No. 2710, Seq. ID No. 2716, Seq. ID No. 2752, Seq. ID No. 2754, Seq. ID No. 2768, Seq. ID No. 2788, Seq. ID No. 2792, Seq. ID No. 2804, Seq. ID No. 2868, Seq. ID No. 2872, Seq. ID No. 2908, Seq. ID No. 2920, Seq. ID No. 2922, Seq. ID No. 2924, Seq. ID No. 2928, Seq. ID No. 2934, Seq. ID No. 2958, Seq. ID No. 2980, Seq. ID No. 3000, Seq. ID No. 3006, Seq. ID No. 3020, Seq. ID No. 3032, Seq. ID No. 3036, Seq. ID No. 3056, Seq. ID No. 3068, Seq. ID No. 3086, Seq. ID No. 3166, Seq. ID No. 3184, Seq. ID No. 3190, Seq. ID No. 3198, Seq. ID No. 3212, Seq. ID No. 3252, Seq. ID No. 3258, Seq. ID No. 3260, Seq. ID No. 3298, Seq. ID No. 3320, Seq. ID No. 3324, Seq. ID No. 3336, Seq. ID No. 3338, Seq. ID No. 3356, Seq. ID No. 3360, Seq. ID No. 3378, Seq. ID No. 3398, Seq. ID No. 3406, Seq. ID No. 3416, Seq. ID No. 3426, Seq. ID No. 3428, Seq. ID No. 3462, Seq. ID No. 3464, Seq. ID No. 3488, Seq. ID No. 3494, Seq. ID No. 3498, Seq. ID No. 3508, Seq. ID No. 3520, Seq. ID No. 3522, Seq. ID No. 3530, Seq. ID No. 3536, Seq. ID No. 3630, Seq. ID No. 3636, Seq. ID No. 3728, Seq. ID No. 3760, Seq. ID No. 3772, Seq. ID No. 3794, Seq. ID No. 3840, Seq. ID No. 3852, Seq. ID No. 3864, Seq. ID No. 3866, Seq. ID No. 3884, Seq. ID No. 3892, Seq. ID No. 3914, Seq. ID No. 3926, Seq. ID No. 3930, Seq. ID No. 3976, Seq. ID No. 3982, Seq. ID No. 4028, Seq. ID No. 4040, Seq. ID No. 4062, Seq. ID No. 4074, Seq. ID No. 4082, Seq. ID No. 4104, Seq. ID No. 4112, Seq. ID No. 4138, Seq. ID No. 4226, Seq. ID No. 4248, Seq. ID No. 4344, Seq. ID No. 4380, Seq. ID No. 4390, Seq. ID No. 4470, Seq. ID No. 4474, Seq. ID No. 4482, Seq. ID No. 4486, Seq. ID No. 4494, Seq. ID No. 4530, Seq. ID No. 4644, Seq. ID No. 4706, Seq. ID No. 4714, Seq. ID No. 4718, Seq. ID No. 4814, Seq. ID No. 4816, Seq. ID No. 4826, Seq. ID No. 4828, Seq. ID No. 4834, Seq. ID No. 4838, Seq. ID No. 4852, Seq. ID No. 4864, Seq. ID No. 4876, Seq. ID No. 4880, Seq. ID No. 4894, Seq. ID No. 4902, Seq. ID No. 4920, Seq. ID No. 4932, Seq. ID No. 4960, Seq. ID No. 4964, Seq. ID No. 4986, Seq. ID No. 5114, Seq. ID No. 5116, Seq. ID No. 5118, Seq. ID No. 5126, Seq. ID No. 5142, Seq. ID No. 5184, Seq. ID No. 5202, Seq. ID No. 5206, Seq. ID No. 5214, Seq. ID No. 5220, Seq. ID No. 5280, Seq. ID No. 5286, Seq. ID No. 5332, Seq. ID No. 5382, Seq. ID No. 5390, Seq. ID No. 5406, Seq. ID No. 5412, Seq. ID No. 5420, Seq. ID No. 5434, Seq. ID No. 5464, Seq. ID No. 5478, Seq. ID No. 5492, Seq. ID No. 5510, Seq. ID No. 5514, Seq. ID No. 5526, Seq. ID No. 5530, Seq. ID No. 5538, Seq. ID No. 5568, Seq. ID No. 5600, Seq. ID No. 5622, Seq. ID No. 5638, Seq. ID No. 5700, Seq. ID No. 5724, Seq. ID No. 5734, Seq. ID No. 5762, Seq. ID No. 5792, Seq. ID No. 5896, Seq. ID No. 5902, Seq. ID No. 5904, Seq. ID No. 5910, Seq. ID No. 5914, Seq. ID No. 5920, Seq. ID No. 5930, Seq. ID No. 5940, Seq. ID No. 6002, Seq. ID No. 6044, Seq. ID No. 6054, Seq. ID No. 6058, Seq. ID No. 6090, Seq. ID No. 6096, Seq. ID No. 6104, Seq. ID No. 6112, Seq. ID No. 6118, Seq. ID No. 6166, Seq. ID No. 6170, Seq. ID No. 6186, Seq. ID No. 6192, Seq. ID No. 6210, Seq. ID No. 6230, Seq. ID No. 6258, Seq. ID No. 6264, Seq. ID No. 6272, Seq. ID No. 6276, Seq. ID No. 6290, Seq. ID No. 6304, Seq. ID No. 6366, Seq. ID No. 6370, Seq. ID No. 6384, and Seq. ID No. 6416.

In another preferred embodiment, the polypeptide is selected from the Alloiococcus otitidis polypeptides, which have been identified by Pfam analysis, and is selected from one of Seq. ID No. 90, Seq. ID No. 146, Seq. ID No. 156, Seq. ID No. 168, Seq. ID No. 174, Seq. ID No. 192, Seq. ID No. 216, Seq. ID No. 220, Seq. ID No. 222, Seq. ID No. 224, Seq. ID No. 226, Seq. ID No. 234, Seq. ID No. 240, Seq. ID No. 252, Seq. ID No. 254, Seq. ID No. 272, Seq. ID No. 292, Seq. ID No. 332, Seq. ID No. 342, Seq. ID No. 346, Seq. ID No. 376, Seq. ID No. 502, Seq. ID No. 506, Seq. ID No. 512, Seq. ID No. 530, Seq. ID No. 568, Seq. ID No. 570, Seq. ID No. 576, Seq. ID No. 594, Seq. ID No. 596, Seq. ID No. 602, Seq. ID No. 638, Seq. ID No. 650, Seq. ID No. 662, Seq. ID No. 670, Seq. ID No. 676, Seq. ID No. 682, Seq. ID No. 684, Seq. ID No. 730, Seq. ID No. 754, Seq. ID No. 780, Seq. ID No. 828, Seq. ID No. 840, Seq. ID No. 896, Seq. ID No. 940, Seq. ID No. 970, Seq. ID No. 986, Seq. ID No. 1016, Seq. ID No. 1040, Seq. ID No. 1076, Seq. ID No. 1138, Seq. ID No. 1156, Seq. ID No. 1164, Seq. ID No. 1176, Seq. ID No. 1224, Seq. ID No. 1232, Seq. ID No. 1238, Seq. ID No. 1242, Seq. ID No. 1262, Seq. ID No. 1332, Seq. ID No. 1340, Seq. ID No. 1370, Seq. ID No. 1478, Seq. ID No. 1518, Seq. ID No. 1532, Seq. ID No. 1540, Seq. ID No. 1550, Seq. ID No. 1564, Seq. ID No. 1568, Seq. ID No. 1572, Seq. ID No. 1574, Seq. ID No. 1590, Seq. ID No. 1596, Seq. ID No. 1648, Seq. ID No. 1658, Seq. ID No. 1772, Seq. ID No. 1786, Seq. ID No. 1790, Seq. ID No. 1820, Seq. ID No. 1822, Seq. ID No. 1970, Seq. ID No. 2016, Seq. ID No. 2048, Seq. ID No. 2088, Seq. ID No. 2090, Seq. ID No. 2094, Seq. ID No. 2116, Seq. ID No. 2118, Seq. ID No. 2128, Seq. ID No. 2142, Seq. ID No. 2180, Seq. ID No. 2208, Seq. ID No. 2224, Seq. ID No. 2254, Seq. ID No. 2276, Seq. ID No. 2302, Seq. ID No. 2316, Seq. ID No. 2330, Seq. ID No. 2344, Seq. ID No. 2350, Seq. ID No. 2364, Seq. ID No. 2382, Seq. ID No. 2392, Seq. ID No. 2418, Seq. ID No. 2422, Seq. ID No. 2424, Seq. ID No. 2430, Seq. ID No. 2482, Seq. ID No. 2488, Seq. ID No. 2490, Seq. ID No. 2492, Seq. ID No. 2510, Seq. ID No. 2520, Seq. ID No. 2526, Seq. ID No. 2548, Seq. ID No. 2554, Seq. ID No. 2564, Seq. ID No. 2588, Seq. ID No. 2600, Seq. ID No. 2608, Seq. ID No. 2614, Seq. ID No. 2652, Seq. ID No. 2676, Seq. ID No. 2682, Seq. ID No. 2688, Seq. ID No. 2694, Seq. ID No. 2700, Seq. ID No. 2704, Seq. ID No. 2710, Seq. ID No. 2712, Seq. ID No. 2728, Seq. ID No. 2754, Seq. ID No. 2820, Seq. ID No. 2822, Seq. ID No. 2824, Seq. ID No. 2868, Seq. ID No. 2872, Seq. ID No. 2908, Seq. ID No. 2920, Seq. ID No. 2958, Seq. ID No. 2972, Seq. ID No. 2980, Seq. ID No. 3000, Seq. ID No. 3006, Seq. ID No. 3020, Seq. ID No. 3036, Seq. ID No. 3066, Seq. ID No. 3086, Seq. ID No. 3150, Seq. ID No. 3166, Seq. ID No. 3184, Seq. ID No. 3204, Seq. ID No. 3212, Seq. ID No. 3252, Seq. ID No. 3260, Seq. ID No. 3320, Seq. ID No. 3324, Seq. ID No. 3336, Seq. ID No. 3338, Seq. ID No. 3348, Seq. ID No. 3360, Seq. ID No. 3378, Seq. ID No. 3380, Seq. ID No. 3400, Seq. ID No. 3426, Seq. ID No. 3484, Seq. ID No. 3496, Seq. ID No. 3498, Seq. ID No. 3520, Seq. ID No. 3530, Seq. ID No. 3600, Seq. ID No. 3612, Seq. ID No. 3616, Seq. ID No. 3620, Seq. ID No. 3664, Seq. ID No. 3740, Seq. ID No. 3760, Seq. ID No. 3772, Seq. ID No. 3798, Seq. ID No. 3826, Seq. ID No. 3842, Seq. ID No. 3856, Seq. ID No. 3864, Seq. ID No. 3902, Seq. ID No. 3930, Seq. ID No. 4016, Seq. ID No. 4018, Seq. ID No. 4056, Seq. ID No. 4076, Seq. ID No. 4086, Seq. ID No. 4090, Seq. ID No. 4100, Seq. ID No. 4106, Seq. ID No. 4130, Seq. ID No. 4204, Seq. ID No. 4244, Seq. ID No. 4252, Seq. ID No. 4344, Seq. ID No. 4384, Seq. ID No. 4476, Seq. ID No. 4482, Seq. ID No. 4488, Seq. ID No. 4492, Seq. ID No. 4534, Seq. ID No. 4628, Seq. ID No. 4706, Seq. ID No. 4714, Seq. ID No. 4718, Seq. ID No. 4720, Seq. ID No. 4730, Seq. ID No. 4790, Seq. ID No. 4792, Seq. ID No. 4794, Seq. ID No. 4804, Seq. ID No. 4814, Seq. ID No. 4816, Seq. ID No. 4826, Seq. ID No. 4834, Seq. ID No. 4838, Seq. ID No. 4876, Seq. ID No. 4880, Seq. ID No. 4920, Seq. ID No. 4932, Seq. ID No. 4948, Seq. ID No. 4986, Seq. ID No. 5078, Seq. ID No. 5116, Seq. ID No. 5118, Seq. ID No. 5128, Seq. ID No. 5142, Seq. ID No. 5194, Seq. ID No. 5216, Seq. ID No. 5220, Seq. ID No. 5276, Seq. ID No. 5382, Seq. ID No. 5390, Seq. ID No. 5410, Seq. ID No. 5412, Seq. ID No. 5476, Seq. ID No. 5492, Seq. ID No. 5514, Seq. ID No. 5520, Seq. ID No. 5526, Seq. ID No. 5530, Seq. ID No. 5538, Seq. ID No. 5558, Seq. ID No. 5582, Seq. ID No. 5600, Seq. ID No. 5638, Seq. ID No. 5686, Seq. ID No. 5724, Seq. ID No. 5756, Seq. ID No. 5762, Seq. ID No. 5896, Seq. ID No. 5902, Seq. ID No. 5904, Seq. ID No. 5912, Seq. ID No. 5914, Seq. ID No. 5920, Seq. ID No. 5924, Seq. ID No. 5930, Seq. ID No. 6002, Seq. ID No. 6044, Seq. ID No. 6054, Seq. ID No. 6056, Seq. ID No. 6090, Seq. ID No. 6096, Seq. ID No. 6112, Seq. ID No. 6118, Seq. ID No. 6166, Seq. ID No. 6170, Seq. ID No. 6188, Seq. ID No. 6196, Seq. ID No. 6220, Seq. ID No. 6234, Seq. ID No. 6270, Seq. ID No. 6276, Seq. ID No. 6280, Seq. ID No. 6288, Seq. ID No. 6298, Seq. ID No. 6300, Seq. ID No. 6306, Seq. ID No. 6312, Seq. ID No. 6370, Seq. ID No. 6380, Seq. ID No. 6588, and Seq. ID No. 6592.

In yet another preferred embodiment, the polypeptide is selected from the Alloiococcus otitidis polypeptides, wherein the polypeptide is identified using proteomics methodology as sharing homology with surface exposed proteins in Streptococcus pneumoniae, and is selected from one of Seq. ID No. 2, Seq. ID No. 6, Seq. ID No. 94, Seq. ID No. 96, Seq. ID No. 104, Seq. ID No. 108, Seq. ID No. 158, Seq. ID No. 224, Seq. ID No. 226, Seq. ID No. 234, Seq. ID No. 240, Seq. ID No. 254, Seq. ID No. 318, Seq. ID No. 322, Seq. ID No. 342, Seq. ID No. 346, Seq. ID No. 354, Seq. ID No. 362, Seq. ID No. 364, Seq. ID No. 366, Seq. ID No. 376, Seq. ID No. 380, Seq. ID No. 384, Seq. ID No. 390, Seq. ID No. 392, Seq. ID No. 398, Seq. ID No. 406, Seq. ID No. 410, Seq. ID No. 416, Seq. ID No. 424, Seq. ID No. 426, Seq. ID No. 428, Seq. ID No. 430, Seq. ID No. 436, Seq. ID No. 438, Seq. ID No. 440, Seq. ID No. 442, Seq. ID No. 444, Seq. ID No. 446, Seq. ID No. 448, Seq. ID No. 450, Seq. ID No. 462, Seq. ID No. 464, Seq. ID No. 466, Seq. ID No. 514, Seq. ID No. 544, Seq. ID No. 552, Seq. ID No. 554, Seq. ID No. 560, Seq. ID No. 596, Seq. ID No. 602, Seq. ID No. 638, Seq. ID No. 650, Seq. ID No. 658, Seq. ID No. 670, Seq. ID No. 676, Seq. ID No. 684, Seq. ID No. 754, Seq. ID No. 930, Seq. ID No. 970, Seq. ID No. 1042, Seq. ID No. 1044, Seq. ID No. 1086, Seq. ID No. 1090, Seq. ID No. 1094, Seq. ID No. 1098, Seq. ID No. 1100, Seq. ID No. 1102, Seq. ID No. 1176, Seq. ID No. 1206, Seq. ID No. 1238, Seq. ID No. 1460, Seq. ID No. 1496, Seq. ID No. 1498, Seq. ID No. 1504, Seq. ID No. 1518, Seq. ID No. 1528, Seq. ID No. 1532, Seq. ID No. 1540, Seq. ID No. 1544, Seq. ID No. 1550, Seq. ID No. 1574, Seq. ID No. 1658, Seq. ID No. 1710, Seq. ID No. 1768, Seq. ID No. 1786, Seq. ID No. 1822, Seq. ID No. 1890, Seq. ID No. 1926, Seq. ID No. 1970, Seq. ID No. 2004, Seq. ID No. 2048, Seq. ID No. 2076, Seq. ID No. 2090, Seq. ID No. 2094, Seq. ID No. 2118, Seq. ID No. 2138, Seq. ID No. 2144, Seq. ID No. 2206, Seq. ID No. 2236, Seq. ID No. 2258, Seq. ID No. 2302, Seq. ID No. 2364, Seq. ID No. 2424, Seq. ID No. 2446, Seq. ID No. 2452, Seq. ID No. 2482, Seq. ID No. 2496, Seq. ID No. 2506, Seq. ID No. 2578, Seq. ID No. 2588, Seq. ID No. 2614, Seq. ID No. 2634, Seq. ID No. 2652, Seq. ID No. 2666, Seq. ID No. 2670, Seq. ID No. 2678, Seq. ID No. 2688, Seq. ID No. 2694, Seq. ID No. 2700, Seq. ID No. 2712, Seq. ID No. 2716, Seq. ID No. 2758, Seq. ID No. 2792, Seq. ID No. 2794, Seq. ID No. 2804, Seq. ID No. 2868, Seq. ID No. 2920, Seq. ID No. 2930, Seq. ID No. 3006, Seq. ID No. 3020, Seq. ID No. 3126, Seq. ID No. 3138, Seq. ID No. 3162, Seq. ID No. 3180, Seq. ID No. 3252, Seq. ID No. 3260, Seq. ID No. 3286, Seq. ID No. 3310, Seq. ID No. 3318, Seq. ID No. 3338, Seq. ID No. 3346, Seq. ID No. 3360, Seq. ID No. 3476, Seq. ID No. 3516, Seq. ID No. 3522, Seq. ID No. 3642, Seq. ID No. 3696, Seq. ID No. 3794, Seq. ID No. 3902, Seq. ID No. 3928, Seq. ID No. 3988, Seq. ID No. 4002, Seq. ID No. 4058, Seq. ID No. 4082, Seq. ID No. 4112, Seq. ID No. 4126, Seq. ID No. 4194, Seq. ID No. 4196, Seq. ID No. 4238, Seq. ID No. 4248, Seq. ID No. 4384, Seq. ID No. 4394, Seq. ID No. 4398, Seq. ID No. 4402, Seq. ID No. 4436, Seq. ID No. 4450, Seq. ID No. 4472, Seq. ID No. 4492, Seq. ID No. 4512, Seq. ID No. 4520, Seq. ID No. 4532, Seq. ID No. 4550, Seq. ID No. 4578, Seq. ID No. 4582, Seq. ID No. 4586, Seq. ID No. 4610, Seq. ID No. 4612, Seq. ID No. 4614, Seq. ID No. 4676, Seq. ID No. 4686, Seq. ID No. 4720, Seq. ID No. 4730, Seq. ID No. 4744, Seq. ID No. 4756, Seq. ID No. 4782, Seq. ID No. 4788, Seq. ID No. 4838, Seq. ID No. 4842, Seq. ID No. 4850, Seq. ID No. 4856, Seq. ID No. 4890, Seq. ID No. 4902, Seq. ID No. 4916, Seq. ID No. 4918, Seq. ID No. 4932, Seq. ID No. 4936, Seq. ID No. 4978, Seq. ID No. 5026, Seq. ID No. 5030, Seq. ID No. 5110, Seq. ID No. 5116, Seq. ID No. 5118, Seq. ID No. 5156, Seq. ID No. 5186, Seq. ID No. 5202, Seq. ID No. 5204, Seq. ID No. 5220, Seq. ID No. 5262, Seq. ID No. 5290, Seq. ID No. 5296, Seq. ID No. 5378, Seq. ID No. 5390, Seq. ID No. 5400, Seq. ID No. 5412, Seq. ID No. 5538, Seq. ID No. 5622, Seq. ID No. 5628, Seq. ID No. 5658, Seq. ID No. 5676, Seq. ID No. 5718, Seq. ID No. 5778, Seq. ID No. 5860, Seq. ID No. 5902, Seq. ID No. 5914, Seq. ID No. 5920, Seq. ID No. 5926, Seq. ID No. 5934, Seq. ID No. 6056, Seq. ID No. 6130, Seq. ID No. 6134, Seq. ID No. 6188, Seq. ID No. 6194, Seq. ID No. 6220, Seq. ID No. 6272, Seq. ID No. 6278, Seq. ID No. 6370, Seq. ID No. 6376, Seq. ID No. 6380, Seq. ID No. 6390, Seq. ID No. 6592, and Seq. ID No. 6606.

In yet another preferred embodiment, the polypeptide is selected from the Alloiococcus otitidis polypeptides identified as having an LPXTG motif, which are covalently attached to the peptidoglycan layer, and is selected from one of Seq. ID No. 504, Seq. ID No. 506, Seq. ID No. 1316, Seq. ID No. 1318, Seq. ID No. 1364, Seq. ID No. 1366, Seq. ID No. 1368, Seq. ID No. 1370, Seq. ID No. 1560, Seq. ID No. 1562, Seq. ID No. 1582, Seq. ID No. 1740, Seq. ID No. 1742, Seq. ID No. 2812, Seq. ID No. 2814, Seq. ID No. 3620, Seq. ID No. 5588, Seq. ID No. 5590, Seq. ID No. 5592, Seq. ID No. 5782, Seq. ID No. 5784, and Seq. ID No. 6104.

In yet another preferred embodiment, the polypeptide is selected from the Alloiococcus otitidis polypeptides identified as lipoproteins using the HMM Lipo algorithm, and is selected from one Seq. ID No. 46, Seq. ID No. 48, Seq. ID No. 172, Seq. ID No. 226, Seq. ID No. 420, Seq. ID No. 514, Seq. ID No. 576, Seq. ID No. 592, Seq. ID No. 594, Seq. ID No. 658, Seq. ID No. 660, Seq. ID No. 670, Seq. ID No. 676, Seq. ID No. 746, Seq. ID No. 748, Seq. ID No. 936, Seq. ID No. 1046, Seq. ID No. 1076, Seq. ID No. 1138, Seq. ID No. 1174, Seq. ID No. 1192, Seq. ID No. 1194, Seq. ID No. 1196, Seq. ID No. 1564, Seq. ID No. 1662, Seq. ID No. 1664, Seq. ID No. 1666, Seq. ID No. 1792, Seq. ID No. 1798, Seq. ID No. 1918, Seq. ID No. 1970, Seq. ID No. 2116, Seq. ID No. 2160, Seq. ID No. 2430, Seq. ID No. 2528, Seq. ID No. 2700, Seq. ID No. 2816, Seq. ID No. 2876, Seq. ID No. 2976, Seq. ID No. 2978, Seq. ID No. 2992, Seq. ID No. 3044, Seq. ID No. 3324, Seq. ID No. 3338, Seq. ID No. 4134, Seq. ID No. 4138, Seq. ID No. 4482, Seq. ID No. 4706, Seq. ID No. 4828, Seq. ID No. 4870, Seq. ID No. 5116, Seq. ID No. 5378, Seq. ID No. 5380, Seq. ID No. 5492, Seq. ID No. 5510, Seq. ID No. 5514, Seq. ID No. 5526, Seq. ID No. 5530, Seq. ID No. 5532, Seq. ID No. 5890, Seq. ID No. 5910, Seq. ID No. 6026, Seq. ID No. 6028, Seq. ID No. 6088, Seq. ID No. 6090, Seq. ID No. 6326, Seq. ID No. 6328, Seq. ID No. 6416, Seq. ID No. 6638, Seq. ID No. 6640, and Seq. ID No. 6646.

In yet another preferred embodiment, the polypeptide is selected from the Alloiococcus otitidis polypeptides, wherein the polypeptide is non-covalently associated with the peptidoglycan layer and is selected from one of Seq. ID No. 3590, Seq. ID No. 3988, Seq. ID No. 5220, and Seq. ID No. 5338.

In yet another preferred embodiment, the polypeptide is selected from the Alloiococcus otitidis polypeptides having an RGDX motif wherein X is not a proline residue, and is selected from one of Seq. ID No. 38, Seq. ID No. 100, Seq. ID No. 108, Seq. ID No. 110, Seq. ID No. 112, Seq. ID No. 352, Seq. ID No. 354, Seq. ID No. 466, Seq. ID No. 468, Seq. ID No. 470, Seq. ID No. 472, Seq. ID No. 684, Seq. ID No. 822, Seq. ID No. 824, Seq. ID No. 1022, Seq. ID No. 1024, Seq. ID No. 1158, Seq. ID No. 1160, Seq. ID No. 1234, Seq. ID No. 1334, Seq. ID No. 1336, Seq. ID No. 1450, Seq. ID No. 1560, Seq. ID No. 1562, Seq. ID No. 1564, Seq. ID No. 1626, Seq. ID No. 1778, Seq. ID No. 1828, Seq. ID No. 1830, Seq. ID No. 1872, Seq. ID No. 1926, Seq. ID No. 2136, Seq. ID No. 2146, Seq. ID No. 2174, Seq. ID No. 2176, Seq. ID No. 2178, Seq. ID No. 2180, Seq. ID No. 2260, Seq. ID No. 2318, Seq. ID No. 2320, Seq. ID No. 2322, Seq. ID No. 2324, Seq. ID No. 2326, Seq. ID No. 2328, Seq. ID No. 2330, Seq. ID No. 2402, Seq. ID No. 2672, Seq. ID No. 2864, Seq. ID No. 2870, Seq. ID No. 2920, Seq. ID No. 2922, Seq. ID No. 2994, Seq. ID No. 3088, Seq. ID No. 3138, Seq. ID No. 3280, Seq. ID No. 3282, Seq. ID No. 3284, Seq. ID No. 3334, Seq. ID No. 3336, Seq. ID No. 3498, Seq. ID No. 3500, Seq. ID No. 3502, Seq. ID No. 3534, Seq. ID No. 3672, Seq. ID No. 3674, Seq. ID No. 3676, Seq. ID No. 3838, Seq. ID No. 3840, Seq. ID No. 3842, Seq. ID No. 3844, Seq. ID No. 3930, Seq. ID No. 3962, Seq. ID No. 4038, Seq. ID No. 4194, Seq. ID No. 4240, Seq. ID No. 4328, Seq. ID No. 4330, Seq. ID No. 4334, Seq. ID No. 4394, Seq. ID No. 4464, Seq. ID No. 4466, Seq. ID No. 4468, Seq. ID No. 4598, Seq. ID No. 4630, Seq. ID No. 4632, Seq. ID No. 4676, Seq. ID No. 4678, Seq. ID No. 4680, Seq. ID No. 4690, Seq. ID No. 4692, Seq. ID No. 4694, Seq. ID No. 4782, Seq. ID No. 4800, Seq. ID No. 4802, Seq. ID No. 4804, Seq. ID No. 4862, Seq. ID No. 5010, Seq. ID No. 5144, Seq. ID No. 5146, Seq. ID No. 5180, Seq. ID No. 5182, Seq. ID No. 5184, Seq. ID No. 5250, Seq. ID No. 5252, Seq. ID No. 5254, Seq. ID No. 5260, Seq. ID No. 5262, Seq. ID No. 5294, Seq. ID No. 5296, Seq. ID No. 5298, Seq. ID No. 5300, Seq. ID No. 5308, Seq. ID No. 5310, Seq. ID No. 5412, Seq. ID No. 5536, Seq. ID No. 5538, Seq. ID No. 5746, Seq. ID No. 5822, Seq. ID No. 5824, Seq. ID No. 5826, Seq. ID No. 6030, Seq. ID No. 6172, Seq. ID No. 6308, Seq. ID No. 6310, and Seq. ID No. 6312.

In another preferred embodiment, the polypeptide is selected from Alloiococcus otitidis polypeptides predicted by BlastP as being involved in capsule biosynthesis and transport, and is selected from one of Seq. ID No. 50, Seq. ID No. 52, Seq. ID No. 54, Seq. ID No. 60, Seq. ID No. 196, Seq. ID No. 690, Seq. ID No. 704, Seq. ID No. 926, Seq. ID No. 1598, Seq. ID No. 1602, Seq. ID No. 1608, Seq. ID No. 1612, Seq. ID No. 1614, Seq. ID No. 1714, Seq. ID No. 2030, Seq. ID No. 2264, Seq. ID No. 2270, Seq. ID No. 2374, Seq. ID No. 2438, Seq. ID No. 2652, Seq. ID No. 2768, Seq. ID No. 2908, Seq. ID No. 3398, Seq. ID No. 3400, Seq. ID No. 3480, Seq. ID No. 3488, Seq. ID No. 3492, Seq. ID No. 3494, Seq. ID No. 3496, Seq. ID No. 3498, Seq. ID No. 3520, Seq. ID No. 3522, Seq. ID No. 3530, Seq. ID No. 3536, Seq. ID No. 3562, Seq. ID No. 3884, Seq. ID No. 3892, Seq. ID No. 3982, and Seq. ID No. 4088.

In another preferred embodiment, the polypeptide is selected from Alloiococcus otitidis polypeptides predicted by BlastP as being localized in the capsule loci region of Alloiococcus otitidis, and is selected from one of Seq. ID No. 46, Seq. ID No. 48, Seq. ID No. 52, Seq. ID No. 54, Seq. ID No. 56, Seq. ID No. 58, Seq. ID No. 60, Seq. ID No. 684, Seq. ID No. 686, Seq. ID No. 688, Seq. ID No. 690, Seq. ID No. 692, Seq. ID No. 694, Seq. ID No. 696, Seq. ID No. 698, Seq. ID No. 700, Seq. ID No. 702, Seq. ID No. 704, Seq. ID No. 706, Seq. ID No. 708, Seq. ID No. 710, Seq. ID No. 712, Seq. ID No. 714, Seq. ID No. 716, Seq. ID No. 718, Seq. ID No. 720, Seq. ID No. 722, Seq. ID No. 724, Seq. ID No. 726, Seq. ID No. 728, Seq. ID No. 730, Seq. ID No. 732, Seq. ID No. 734, Seq. ID No. 736, Seq. ID No. 738, Seq. ID No. 740, Seq. ID No. 742, Seq. ID No. 744, Seq. ID No. 746, Seq. ID No. 748, Seq. ID No. 750, Seq. ID No. 752, Seq. ID No. 754, Seq. ID No. 3390, Seq. ID No. 3392, Seq. ID No. 3394, Seq. ID No. 3396, Seq. ID No. 3398, Seq. ID No. 3400, Seq. ID No. 3402, Seq. ID No. 3404, Seq. ID No. 3406, Seq. ID No. 3408, Seq. ID No. 3410, Seq. ID No. 3412, Seq. ID No. 3414, Seq. ID No. 3416, Seq. ID No. 3418, Seq. ID No. 3420, Seq. ID No. 3422, Seq. ID No. 3424, Seq. ID No. 3426, Seq. ID No. 3428, Seq. ID No. 3430, Seq. ID No. 3432, Seq. ID No. 3434, Seq. ID No. 3436, Seq. ID No. 3438, Seq. ID No. 3440, Seq. ID No. 3442, Seq. ID No. 3444, Seq. ID No. 3446, Seq. ID No. 3448, Seq. ID No. 3450, Seq. ID No. 3452, Seq. ID No. 3454, Seq. ID No. 3456, Seq. ID No. 3458, Seq. ID No. 3460, Seq. ID No. 3462, Seq. ID No. 3464, Seq. ID No. 3466, Seq. ID No. 3468, Seq. ID No. 3470, Seq. ID No. 3472, Seq. ID No. 3474, Seq. ID No. 3476, Seq. ID No. 3478, Seq. ID No. 3480, Seq. ID No. 3482, Seq. ID No. 3484, Seq. ID No. 3486, Seq. ID No. 3488, Seq. ID No. 3490, Seq. ID No. 3492, Seq. ID No. 3494, Seq. ID No. 3496, Seq. ID No. 3498, Seq. ID No. 3500, Seq. ID No. 3502, Seq. ID No. 3504, Seq. ID No. 3506, Seq. ID No. 3508, Seq. ID No. 3510, Seq. ID No. 3512, Seq. ID No. 3514, Seq. ID No. 3516, Seq. ID No. 3518, Seq. ID No. 3520, Seq. ID No. 3522, Seq. ID No. 3524, Seq. ID No. 3526, Seq. ID No. 3528, Seq. ID No. 3530, Seq. ID No. 3532, Seq. ID No. 3534, Seq. ID No. 3536, Seq. ID No. 3538, Seq. ID No. 3540, Seq. ID No. 3542, Seq. ID No. 3544, Seq. ID No. 3546, Seq. ID No. 3548, Seq. ID No. 3550, Seq. ID No. 3552, Seq. ID No. 3554, Seq. ID No. 3556, Seq. ID No. 3558, Seq. ID No. 3560, and Seq. ID No. 3562.

In another preferred embodiment, the polypeptide is selected from the Alloiococcus otitidis polypeptides identified by BlastP analysis to be associated with sporulation, and is selected from one of Seq. ID No. 1138, Seq. ID No. 1456, Seq. ID No. 2138, Seq. ID No. 2142, Seq. ID No. 2166, Seq. ID No. 2180, Seq. ID No. 2302, Seq. ID No. 2754, Seq. ID No. 4212, Seq. ID No. 4252, Seq. ID No. 4328, Seq. ID No. 4570, Seq. ID No. 4720, and Seq. ID No. 4730.

In another preferred embodiment, the polypeptide is selected from the Alloiococcus otitidis polypeptides identified by BlastP analysis as being encoded by unique ORFs, and is selected from one of Seq. ID No. 8, Seq. ID No. 20, Seq. ID No. 22, Seq. ID No. 24, Seq. ID No. 32, Seq. ID No. 40, Seq. ID No. 66, Seq. ID No. 68, Seq. ID No. 70, Seq. ID No. 92, Seq. ID No. 100, Seq. ID No. 106, Seq. ID No. 114, Seq. ID No. 116, Seq. ID No. 124, Seq. ID No. 126, Seq. ID No. 128, Seq. ID No. 132, Seq. ID No. 134, Seq. ID No. 140, Seq. ID No. 152, Seq. ID No. 160, Seq. ID No. 162, Seq. ID No. 164, Seq. ID No. 166, Seq. ID No. 170, Seq. ID No. 186, Seq. ID No. 190, Seq. ID No. 194, Seq. ID No. 230, Seq. ID No. 232, Seq. ID No. 256, Seq. ID No. 258, Seq. ID No. 260, Seq. ID No. 276, Seq. ID No. 338, Seq. ID No. 340, Seq. ID No. 348, Seq. ID No. 350, Seq. ID No. 358, Seq. ID No. 360, Seq. ID No. 370, Seq. ID No. 372, Seq. ID No. 378, Seq. ID No. 386, Seq. ID No. 388, Seq. ID No. 394, Seq. ID No. 400, Seq. ID No. 402, Seq. ID No. 404, Seq. ID No. 408, Seq. ID No. 420, Seq. ID No. 432, Seq. ID No. 478, Seq. ID No. 496, Seq. ID No. 508, Seq. ID No. 520, Seq. ID No. 542, Seq. ID No. 546, Seq. ID No. 548, Seq. ID No. 550, Seq. ID No. 558, Seq. ID No. 590, Seq. ID No. 618, Seq. ID No. 620, Seq. ID No. 634, Seq. ID No. 640, Seq. ID No. 642, Seq. ID No. 644, Seq. ID No. 646, Seq. ID No. 648, Seq. ID No. 652, Seq. ID No. 654, Seq. ID No. 656, Seq. ID No. 668, Seq. ID No. 692, Seq. ID No. 694, Seq. ID No. 696, Seq. ID No. 698, Seq. ID No. 716, Seq. ID No. 718, Seq. ID No. 720, Seq. ID No. 732, Seq. ID No. 734, Seq. ID No. 742, Seq. ID No. 744, Seq. ID No. 746, Seq. ID No. 748, Seq. ID No. 750, Seq. ID No. 752, Seq. ID No. 756, Seq. ID No. 758, Seq. ID No. 760, Seq. ID No. 762, Seq. ID No. 764, Seq. ID No. 766, Seq. ID No. 768, Seq. ID No. 802, Seq. ID No. 804, Seq. ID No. 806, Seq. ID No. 808, Seq. ID No. 810, Seq. ID No. 812, Seq. ID No. 814, Seq. ID No. 834, Seq. ID No. 842, Seq. ID No. 850, Seq. ID No. 852, Seq. ID No. 854, Seq. ID No. 856, Seq. ID No. 888, Seq. ID No. 890, Seq. ID No. 892, Seq. ID No. 904, Seq. ID No. 906, Seq. ID No. 910, Seq. ID No. 938, Seq. ID No. 942, Seq. ID No. 944, Seq. ID No. 952, Seq. ID No. 958, Seq. ID No. 974, Seq. ID No. 976, Seq. ID No. 988, Seq. ID No. 1006, Seq. ID No. 1008, Seq. ID No. 1010, Seq. ID No. 1012, Seq. ID No. 1020, Seq. ID No. 1026, Seq. ID No. 1028, Seq. ID No. 1046, Seq. ID No. 1048, Seq. ID No. 1072, Seq. ID No. 1074, Seq. ID No. 1078, Seq. ID No. 1106, Seq. ID No. 1108, Seq. ID No. 1112, Seq. ID No. 1132, Seq. ID No. 1136, Seq. ID No. 1140, Seq. ID No. 1152, Seq. ID No. 1154, Seq. ID No. 1166, Seq. ID No. 1168, Seq. ID No. 1170, Seq. ID No. 1172, Seq. ID No. 1174, Seq. ID No. 1182, Seq. ID No. 1184, Seq. ID No. 1186, Seq. ID No. 1188, Seq. ID No. 1190, Seq. ID No. 1192, Seq. ID No. 1194, Seq. ID No. 1196, Seq. ID No. 1198, Seq. ID No. 1200, Seq. ID No. 1202, Seq. ID No. 1204, Seq. ID No. 1208, Seq. ID No. 1214, Seq. ID No. 1228, Seq. ID No. 1230, Seq. ID No. 1240, Seq. ID No. 1244, Seq. ID No. 1246, Seq. ID No. 1248, Seq. ID No. 1250, Seq. ID No. 1260, Seq. ID No. 1266, Seq. ID No. 1268, Seq. ID No. 1270, Seq. ID No. 1272, Seq. ID No. 1274, Seq. ID No. 1276, Seq. ID No. 1278, Seq. ID No. 1280, Seq. ID No. 1282, Seq. ID No. 1284, Seq. ID No. 1286, Seq. ID No. 1288, Seq. ID No. 1290, Seq. ID No. 1292, Seq. ID No. 1294, Seq. ID No. 1296, Seq. ID No. 1298, Seq. ID No. 1300, Seq. ID No. 1302, Seq. ID No. 1304, Seq. ID No. 1306, Seq. ID No. 1308, Seq. ID No. 1310, Seq. ID No. 1312, Seq. ID No. 1314, Seq. ID No. 1320, Seq. ID No. 1322, Seq. ID No. 1324, Seq. ID No. 1326, Seq. ID No. 1328, Seq. ID No. 1338, Seq. ID No. 1342, Seq. ID No. 1344, Seq. ID No. 1348, Seq. ID No. 1350, Seq. ID No. 1352, Seq. ID No. 1354, Seq. ID No. 1356, Seq. ID No. 1372, Seq. ID No. 1380, Seq. ID No. 1382, Seq. ID No. 1390, Seq. ID No. 1398, Seq. ID No. 1400, Seq. ID No. 1402, Seq. ID No. 1418, Seq. ID No. 1420, Seq. ID No. 1422, Seq. ID No. 1424, Seq. ID No. 1440, Seq. ID No. 1446, Seq. ID No. 1448, Seq. ID No. 1458, Seq. ID No. 1466, Seq. ID No. 1480, Seq. ID No. 1512, Seq. ID No. 1552, Seq. ID No. 1554, Seq. ID No. 1556, Seq. ID No. 1558, Seq. ID No. 1580, Seq. ID No. 1610, Seq. ID No. 1622, Seq. ID No. 1624, Seq. ID No. 1626, Seq. ID No. 1628, Seq. ID No. 1630, Seq. ID No. 1632, Seq. ID No. 1634, Seq. ID No. 1640, Seq. ID No. 1644, Seq. ID No. 1656, Seq. ID No. 1668, Seq. ID No. 1672, Seq. ID No. 1694, Seq. ID No. 1696, Seq. ID No. 1702, Seq. ID No. 1704, Seq. ID No. 1706, Seq. ID No. 1722, Seq. ID No. 1724, Seq. ID No. 1732, Seq. ID No. 1738, Seq. ID No. 1744, Seq. ID No. 1760, Seq. ID No. 1764, Seq. ID No. 1774, Seq. ID No. 1776, Seq. ID No. 1778, Seq. ID No. 1780, Seq. ID No. 1782, Seq. ID No. 1800, Seq. ID No. 1802, Seq. ID No. 1804, Seq. ID No. 1808, Seq. ID No. 1824, Seq. ID No. 1832, Seq. ID No. 1852, Seq. ID No. 1858, Seq. ID No. 1876, Seq. ID No. 1900, Seq. ID No. 1916, Seq. ID No. 1928, Seq. ID No. 1930, Seq. ID No. 1974, Seq. ID No. 1978, Seq. ID No. 1982, Seq. ID No. 2006, Seq. ID No.;2008, Seq. ID No. 2010, Seq. ID No. 2012, Seq. ID No. 2018, Seq. ID No. 2020, Seq. ID No. 2024, Seq. ID No. 2026, Seq. ID No. 2028, Seq. ID No. 2040, Seq. ID No. 2042, Seq. ID No. 2044, Seq. ID No. 2046, Seq. ID No. 2050, Seq. ID No. 2082, Seq. ID No. 2084, Seq. ID No. 2086, Seq. ID No. 2106, Seq. ID No. 2108, Seq. ID No. 2110, Seq. ID No. 2112, Seq. ID No. 2130, Seq. ID No. 2132, Seq. ID No. 2134, Seq. ID No. 2146, Seq. ID No. 2156, Seq. ID No. 2158, Seq. ID No. 2160, Seq. ID No. 2162, Seq. ID No. 2172, Seq. ID No. 2188, Seq. ID No. 2190, Seq. ID No. 2202, Seq. ID No. 2204, Seq. ID No. 2210, Seq. ID No. 2212, Seq. ID No. 2220, Seq. ID No. 2226, Seq. ID No. 2244, Seq. ID No. 2246, Seq. ID No. 2248, Seq. ID No. 2250, Seq. ID No. 2252, Seq. ID No. 2256, Seq. ID No. 2278, Seq. ID No. 2280, Seq. ID No. 2342, Seq. ID No. 2352, Seq. ID No. 2354, Seq. ID No. 2356, Seq. ID No. 2358, Seq. ID No. 2360, Seq. ID No. 2366, Seq. ID No. 2368, Seq. ID No. 2372, Seq. ID No. 2373, Seq. ID No. 2394, Seq. ID No. 2396, Seq. ID No. 2426, Seq. ID No. 2428, Seq. ID No. 2432, Seq. ID No. 2440, Seq. ID No. 2442, Seq. ID No. 2444, Seq. ID No. 2448, Seq. ID No. 2460, Seq. ID No. 2480, Seq. ID No. 2494, Seq. ID No. 2502, Seq. ID No. 2504, Seq. ID No. 2522, Seq. ID No. 2524, Seq. ID No. 2528, Seq. ID No. 2592, Seq. ID No. 2602, Seq. ID No. 2604, Seq. ID No. 2612, Seq. ID No. 2628, Seq. ID No. 2630, Seq. ID No. 2654, Seq. ID No. 2662, Seq. ID No. 2692, Seq. ID No. 2696, Seq. ID No. 2698, Seq. ID No. 2718, Seq. ID No. 2730, Seq. ID No. 2732, Seq. ID No. 2734, Seq. ID No. 2736, Seq. ID No. 2740, Seq. ID No. 2742, Seq. ID No. 2744, Seq. ID No. 2778, Seq. ID No. 2780, Seq. ID No. 2782, Seq. ID No. 2784, Seq. ID No. 2802, Seq. ID No. 2806, Seq. ID No. 2808, Seq. ID No. 2810, Seq. ID No. 2812, Seq. ID No. 2814, Seq. ID No. 2816, Seq. ID No. 2818, Seq. ID No. 2820, Seq. ID No. 2830, Seq. ID No. 2832, Seq. ID No. 2834, Seq. ID No. 2836, Seq. ID No. 2838, Seq. ID No. 2840, Seq. ID No. 2842, Seq. ID No. 2846, Seq. ID No. 2850, Seq. ID No. 2852, Seq. ID No. 2854, Seq. ID No. 2856, Seq. ID No. 2858, Seq. ID No. 2860, Seq. ID No. 2862, Seq. ID No. 2866, Seq. ID No. 2874, Seq. ID No. 2876, Seq. ID No. 2878, Seq. ID No. 2884, Seq. ID No. 2886, Seq. ID No. 2888, Seq. ID No. 2890, Seq. ID No. 2894, Seq. ID No. 2896, Seq. ID No. 2904, Seq. ID No. 2910, Seq. ID No. 2936, Seq. ID No. 2938, Seq. ID No. 2940, Seq. ID No. 2942, Seq. ID No. 2944, Seq. ID No. 2946, Seq. ID No. 2948, Seq. ID No. 2950, Seq. ID No. 2952, Seq. ID No. 2954, Seq. ID No. 2966, Seq. ID No. 2968, Seq. ID No. 2974, Seq. ID No. 2976, Seq. ID No. 2978, Seq. ID No. 2982, Seq. ID No. 2984, Seq. ID No. 2986, Seq. ID No. 2988, Seq. ID No. 2990, Seq. ID No. 2992, Seq. ID No. 3002, Seq. ID No. 3008, Seq. ID No. 3010, Seq. ID No. 3012, Seq. ID No. 3022, Seq. ID No. 3024, Seq. ID No. 3042, Seq. ID No. 3046, Seq. ID No. 3072, Seq. ID No. 3088, Seq. ID No. 3106, Seq. ID No. 3108, Seq. ID No. 3120, Seq. ID No. 3122, Seq. ID No. 3124, Seq. ID No. 3152, Seq. ID No. 3154, Seq. ID No. 3168, Seq. ID No. 3174, Seq. ID No. 3176, Seq. ID No. 3178, Seq. ID No. 3182, Seq. ID No. 3194, Seq. ID No. 3214, Seq. ID No. 3216, Seq. ID No. 3218, Seq. ID No. 3220, Seq. ID No. 3222, Seq. ID No. 3224, Seq. ID No. 3232, Seq. ID No. 3234, Seq. ID No. 3236, Seq. ID No. 3238, Seq. ID No. 3240, Seq. ID No. 3242, Seq. ID No. 3244, Seq. ID No. 3246, Seq. ID No. 3248, Seq. ID No. 3250, Seq. ID No. 3262, Seq. ID No. 3264, Seq. ID No. 3266, Seq. ID No. 3268, Seq. ID No. 3270, Seq. ID No. 3272, Seq. ID No. 3274, Seq. ID No. 3276, Seq. ID No. 3278, Seq. ID No. 3326, Seq. ID No. 3332, Seq. ID No. 3340, Seq. ID No. 3342, Seq. ID No. 3350, Seq. ID No. 3362, Seq. ID No. 3364, Seq. ID No. 3370, Seq. ID No. 3372, Seq. ID No. 3390, Seq. ID No. 3404, Seq. ID No. 3418, Seq. ID No. 3420, Seq. ID No. 3422, Seq. ID No. 3434, Seq. ID No. 3438, Seq. ID No. 3442, Seq. ID No. 3444, Seq. ID No. 3446, Seq. ID No. 3448, Seq. ID No. 3474, Seq. ID No. 3482, Seq. ID No. 3484, Seq. ID No. 3490, Seq. ID No. 3504, Seq. ID No. 3506, Seq. ID No. 3510, Seq. ID No. 3512, Seq. ID No. 3524, Seq. ID No. 3526, Seq. ID No. 3532, Seq. ID No. 3534, Seq. ID No. 3540, Seq. ID No. 3542, Seq. ID No. 3544, Seq. ID No. 3546, Seq. ID No. 3548, Seq. ID No. 3556, Seq. ID No. 3558, Seq. ID No. 3564, Seq. ID No. 3566, Seq. ID No. 3572, Seq. ID No. 3574, Seq. ID No. 3582, Seq. ID No. 3588, Seq. ID No. 3592, Seq. ID No. 3594, Seq. ID No. 3596, Seq. ID No. 3612, Seq. ID No. 3614, Seq. ID No. 3618, Seq. ID No. 3620, Seq. ID No. 3622, Seq. ID No. 3624, Seq. ID No. 3626, Seq. ID No. 3632, Seq. ID No. 3638, Seq. ID No. 3640, Seq. ID No. 3656, Seq. ID No. 3658, Seq. ID No. 3660, Seq. ID No. 3662, Seq. ID No. 3678, Seq. ID No. 3680, Seq. ID No. 3684, Seq. ID No. 3686, Seq. ID No. 3702, Seq. ID No. 3704, Seq. ID No. 3718, Seq. ID No. 3720, Seq. ID No. 3722, Seq. ID No. 3724, Seq. ID No. 3730, Seq. ID No. 3732, Seq. ID No. 3734, Seq. ID No. 3736, Seq. ID No. 3738, Seq. ID No. 3740, Seq. ID No. 3742, Seq. ID No. 3750, Seq. ID No. 3752, Seq. ID No. 3754, Seq. ID No. 3756, Seq. ID No. 3762, Seq. ID No. 3782, Seq. ID No. 3784, Seq. ID No. 3786, Seq. ID No. 3788, Seq. ID No. 3790, Seq. ID No. 3792, Seq. ID No. 3800, Seq. ID No. 3808, Seq. ID No. 3810, Seq. ID No. 3812, Seq. ID No. 3814, Seq. ID No. 3816, Seq. ID No. 3818, Seq. ID No. 3828, Seq. ID No. 3830, Seq. ID No. 3832, Seq. ID No. 3834, Seq. ID No. 3836, Seq. ID No. 3850, Seq. ID No. 3880, Seq. ID No. 3882, Seq. ID No. 3896, Seq. ID No. 3904, Seq. ID No. 3910, Seq. ID No. 3916, Seq. ID No. 3940, Seq. ID No. 3960, Seq. ID No. 3962, Seq. ID No. 3964, Seq. ID No. 3966, Seq. ID No. 3974, Seq. ID No. 3978, Seq. ID No. 3980, Seq. ID No. 3994, Seq. ID No. 3996, Seq. ID No. 3998, Seq. ID No. 4006, Seq. ID No. 4008, Seq. ID No. 4010, Seq. ID No. 4042, Seq. ID No. 4044, Seq. ID No. 4046, Seq. ID No. 4070, Seq. ID No. 4078, Seq. ID No. 4098, Seq. ID No. 4114, Seq. ID No. 4124, Seq. ID No. 4132, Seq. ID No. 4134, Seq. ID No. 4136, Seq. ID No. 4140, Seq. ID No. 4152, Seq. ID No. 4154, Seq. ID No. 4156, Seq. ID No. 4182, Seq. ID No. 4222, Seq. ID No. 4242, Seq. ID No. 4254, Seq. ID No. 4258, Seq. ID No. 4260, Seq. ID No. 4262, Seq. ID No. 4264, Seq. ID No. 4266, Seq. ID No. 4270, Seq. ID No. 4276, Seq. ID No. 4278, Seq. ID No. 4280, Seq. ID No. 4282, Seq. ID No. 4286, Seq. ID No. 4288, Seq. ID No. 4298, Seq. ID No. 4306, Seq. ID No. 4308, Seq. ID No. 4364, Seq. ID No. 4366, Seq. ID No. 4372, Seq. ID No. 4374, Seq. ID No. 4396, Seq. ID No. 4400, Seq. ID No. 4404, Seq. ID No. 4426, Seq. ID No. 4438, Seq. ID No. 4440, Seq. ID No. 4442, Seq. ID No. 4454, Seq. ID No. 4456, Seq. ID No. 4514, Seq. ID No. 4516, Seq. ID No. 4552, Seq. ID No. 4562, Seq. ID No. 4574, Seq. ID No. 4576, Seq. ID No. 4588, Seq. ID No. 4590, Seq. ID No. 4592, Seq. ID No. 4594, Seq. ID No. 4596, Seq. ID No. 4618, Seq. ID No. 4620, Seq. ID No. 4622, Seq. ID No. 4626, Seq. ID No. 4628, Seq. ID No. 4634, Seq. ID No. 4636, Seq. ID No. 4638, Seq. ID No. 4640, Seq. ID No. 4660, Seq. ID No. 4682, Seq. ID No. 4688, Seq. ID No. 4696, Seq. ID No. 4700, Seq. ID No. 4702, Seq. ID No. 4722, Seq. ID No. 4736, Seq. ID No. 4738, Seq. ID No. 4742, Seq. ID No. 4784, Seq. ID No. 4790, Seq. ID No. 4806, Seq. ID No. 4812, Seq. ID No. 4818, Seq. ID No. 4820, Seq. ID No. 4822, Seq. ID No. 4824, Seq. ID No. 4836, Seq. ID No. 4854, Seq. ID No. 4886, Seq. ID No. 4914, Seq. ID No. 4924, Seq. ID No. 4950, Seq. ID No. 4952, Seq. ID No. 4976, Seq. ID No. 4982, Seq. ID No. 4984, Seq. ID No. 4988, Seq. ID No. 4994, Seq. ID No. 5000, Seq. ID No. 5002, Seq. ID No. 5012, Seq. ID No. 5014, Seq. ID No. 5024, Seq. ID No. 5032, Seq. ID No. 5034, Seq. ID No. 5036, Seq. ID No. 5038, Seq. ID No. 5042, Seq. ID No. 5044, Seq. ID No. 5054, Seq. ID No. 5062, Seq. ID No. 5064, Seq. ID No. 5086, Seq. ID No. 5094, Seq. ID No. 5152, Seq. ID No. 5154, Seq. ID No. 5160, Seq. ID No. 5162, Seq. ID No. 5168, Seq. ID No. 5174, Seq. ID No. 5176, Seq. ID No. 5178, Seq. ID No. 5190, Seq. ID No. 5192, Seq. ID No. 5194, Seq. ID No. 5198, Seq. ID No. 5200, Seq. ID No. 5222, Seq. ID No. 5224, Seq. ID No. 5226, Seq. ID No. 5228, Seq. ID No. 5238, Seq. ID No. 5268, Seq. ID No. 5270, Seq. ID No. 5272, Seq. ID No. 5282, Seq. ID No. 5302, Seq. ID No. 5304, Seq. ID No. 5316, Seq. ID No. 5322, Seq. ID No. 5328, Seq. ID No. 5348, Seq. ID No. 5386, Seq. ID No. 5446, Seq. ID No. 5448, Seq. ID No. 5460, Seq. ID No. 5466, Seq. ID No. 5468, Seq. ID No. 5480, Seq. ID No. 5482, Seq. ID No. 5486, Seq. ID No. 5488, Seq. ID No. 5516, Seq. ID No. 5518, Seq. ID No. 5534, Seq. ID No. 5544, Seq. ID No. 5546, Seq. ID No. 5550, Seq. ID No. 5570, Seq. ID No. 5578, Seq. ID No. 5594, Seq. ID No. 5596, Seq. ID No. 5610, Seq. ID No. 5624, Seq. ID No. 5626, Seq. ID No. 5640, Seq. ID No. 5642, Seq. ID No. 5644, Seq. ID No. 5650, Seq. ID No. 5660, Seq. ID No. 5662, Seq. ID No. 5664, Seq. ID No. 5668, Seq. ID No. 5672, Seq. ID No. 5688, Seq. ID No. 5690, Seq. ID No. 5704, Seq. ID No. 5706, Seq. ID No. 5710, Seq. ID No. 5712, Seq. ID No. 5738, Seq. ID No. 5740, Seq. ID No. 5746, Seq. ID No. 5752, Seq. ID No. 5764, Seq. ID No. 5798, Seq. ID No. 5812, Seq. ID No. 5814, Seq. ID No. 5816, Seq. ID No. 5828, Seq. ID No. 5830, Seq. ID No. 5844, Seq. ID No. 5846, Seq. ID No. 5874, Seq. ID No. 5880, Seq. ID No. 5884, Seq. ID No. 5886, Seq. ID No. 5888, Seq. ID No. 5890, Seq. ID No. 5892, Seq. ID No. 5932, Seq. ID No. 5956, Seq. ID No. 5964, Seq. ID No. 5966, Seq. ID No. 5976, Seq. ID No. 5984, Seq. ID No. 5986, Seq. ID No. 5988, Seq. ID No. 5990, Seq. ID No. 6006, Seq. ID No. 6014, Seq. ID No. 6032, Seq. ID No. 6036, Seq. ID No. 6046, Seq. ID No. 6060, Seq. ID No. 6062, Seq. ID No. 6064, Seq. ID No. 6074, Seq. ID No. 6082, Seq. ID No. 6092, Seq. ID No. 6094, Seq. ID No. 6128, Seq. ID No. 6160, Seq. ID No. 6198, Seq. ID No. 6200, Seq. ID No. 6204, Seq. ID No. 6218, Seq. ID No. 6222, Seq. ID No. 6224, Seq. ID No. 6238, Seq. ID No. 6240, Seq. ID No. 6242, Seq. ID No. 6246, Seq. ID No. 6248, Seq. ID No. 6250, Seq. ID No. 6260, Seq. ID No. 6266, Seq. ID No. 6268, Seq. ID No. 6274, Seq. ID No. 6284, Seq. ID No. 6286, Seq. ID No. 6324, Seq. ID No. 6326, Seq. ID No. 6328, Seq. ID No. 6362, Seq. ID No. 6364, Seq. ID No. 6378, Seq. ID No. 6382, Seq. ID No. 6402, Seq. ID No. 6404, Seq. ID No. 6420, Seq. ID No. 6426, Seq. ID No. 6428, Seq. ID No. 6430, Seq. ID No. 6432, Seq. ID No. 6434, Seq. ID No. 6436, Seq. ID No. 6438, Seq. ID No. 6440, Seq. ID No. 6448, Seq. ID No. 6450, Seq. ID No. 6452, Seq. ID No. 6454, Seq. ID No. 6456, Seq. ID No. 6458, Seq. ID No. 6466, Seq. ID No. 6468, Seq. ID No. 6470, Seq. ID No. 6472, Seq. ID No. 6474, Seq. ID No. 6476, Seq. ID No. 6478, Seq. ID No. 6480, Seq. ID No. 6482, Seq. ID No. 6484, Seq. ID No. 6486, Seq. ID No. 6488, Seq. ID No. 6490, Seq. ID No. 6492, Seq. ID No. 6496, Seq. ID No. 6498, Seq. ID No. 6508, Seq. ID No. 6518, Seq. ID No. 6520, Seq. ID No. 6522, Seq. ID No. 6524, Seq. ID No. 6526, Seq. ID No. 6528, Seq. ID No. 6530, Seq. ID No. 6534, Seq. ID No. 6540, Seq. ID No. 6542, Seq. ID No. 6544, Seq. ID No. 6556, Seq. ID No. 6558, Seq. ID No. 6562, Seq. ID No. 6564, Seq. ID No. 6566, Seq. ID No. 6568, Seq. ID No. 6570, Seq. ID No. 6572, Seq. ID No. 6574, Seq. ID No. 6582, Seq. ID No. 6584, Seq. ID No. 6632, Seq. ID No. 6636, Seq. ID No. 6638, Seq. ID No. 6640, Seq. ID No. 6644, Seq. ID No. 6646, Seq. ID No. 6648, and Seq. ID No. 6650.

In yet another preferred embodiment, the polypeptide is selected from the Alloiococcus otitidis polypeptides identified by Glimmer™ ORF finder program, and is selected from one of Seq. ID No. 2, Seq. ID No. 6, Seq. ID No. 12, Seq. ID No. 14, Seq. ID No. 16, Seq. ID No. 18, Seq. ID No. 26, Seq. ID No. 28, Seq. ID No. 30, Seq. ID No. 36, Seq. ID No. 38, Seq. ID No. 42, Seq. ID No. 44, Seq. ID No. 48, Seq. ID No. 52, Seq. ID No. 54, Seq. ID No. 60, Seq. ID No. 64, Seq. ID No. 70, Seq. ID No. 74, Seq. ID No. 76, Seq. ID No. 82, Seq. ID No. 84, Seq. ID No. 90, Seq. ID No. 94, Seq. ID No. 98, Seq. ID No. 104, Seq. ID No. 112, Seq. ID No. 122, Seq. ID No. 126, Seq. ID No. 130, Seq. ID No. 136, Seq. ID No. 138, Seq. ID No. 146, Seq. ID No. 150, Seq. ID No. 158, Seq. ID No. 168, Seq. ID No. 174, Seq. ID No. 184, Seq. ID No. 188, Seq. ID No. 190, Seq. ID No. 192, Seq. ID No. 196, Seq. ID No. 198, Seq. ID No. 204, Seq. ID No. 208, Seq. ID No. 216, Seq. ID No. 220, Seq. ID No. 224, Seq. ID No. 226, Seq. ID No. 228, Seq. ID No. 232, Seq. ID No. 234, Seq. ID No. 240, Seq. ID No. 242, Seq. ID No. 248, Seq. ID No. 254, Seq. ID No. 262, Seq. ID No. 266, Seq. ID No. 270, Seq. ID No. 274, Seq. ID No. 276, Seq. ID No. 278, Seq. ID No. 284, Seq. ID No. 292, Seq. ID No. 294, Seq. ID No. 298, Seq. ID No. 302, Seq. ID No. 308, Seq. ID No. 310, Seq. ID No. 314, Seq. ID No. 318, Seq. ID No. 322, Seq. ID No. 326, Seq. ID No. 336, Seq. ID No. 344, Seq. ID No. 346, Seq. ID No. 354, Seq. ID No. 356, Seq. ID No. 362, Seq. ID No. 364, Seq. ID No. 368, Seq. ID No. 376, Seq. ID No. 380, Seq. ID No. 384, Seq. ID No. 390, Seq. ID No. 392, Seq. ID No. 398, Seq. ID No. 406, Seq. ID No. 412, Seq. ID No. 416, Seq. ID No. 418, Seq. ID No. 424, Seq. ID No. 426, Seq. ID No. 428, Seq. ID No. 430, Seq. ID No. 436, Seq. ID No. 438, Seq. ID No. 440, Seq. ID No. 442, Seq. ID No. 446, Seq. ID No. 448, Seq. ID No. 454, Seq. ID No. 462, Seq. ID No. 464, Seq. ID No. 472, Seq. ID No. 486, Seq. ID No. 502, Seq. ID No. 506, Seq. ID No. 512, Seq. ID No. 516, Seq. ID No. 518, Seq. ID No. 522, Seq. ID No. 524, Seq. ID No. 532, Seq. ID No. 534, Seq. ID No. 540, Seq. ID No. 542, Seq. ID No. 544, Seq. ID No. 552, Seq. ID No. 556, Seq. ID No. 562, Seq. ID No. 566, Seq. ID No. 570, Seq. ID No. 576, Seq. ID No. 580, Seq. ID No. 588, Seq. ID No. 594, Seq. ID No. 598, Seq. ID No. 604, Seq. ID No. 606, Seq. ID No. 610, Seq. ID No. 612, Seq. ID No. 614, Seq. ID No. 616, Seq. ID No. 624, Seq. ID No. 628, Seq. ID No. 630, Seq. ID No. 632, Seq. ID No. 636, Seq. ID No. 638, Seq. ID No. 648, Seq. ID No. 650, Seq. ID No. 662, Seq. ID No. 670, Seq. ID No. 674, Seq. ID No. 676, Seq. ID No. 682, Seq. ID No. 684, Seq. ID No. 690, Seq. ID No. 702, Seq. ID No. 704, Seq. ID No. 710, Seq. ID No. 714, Seq. ID No. 730, Seq. ID No. 734, Seq. ID No. 736, Seq. ID No. 738, Seq. ID No. 742, Seq. ID No. 748, Seq. ID No. 752, Seq. ID No. 754, Seq. ID No. 760, Seq. ID No. 766, Seq. ID No. 768, Seq. ID No. 772, Seq. ID No. 782, Seq. ID No. 784, Seq. ID No. 788, Seq. ID No. 792, Seq. ID No. 796, Seq. ID No. 800, Seq. ID No. 808, Seq. ID No. 818, Seq. ID No. 820, Seq. ID No. 824, Seq. ID No. 828, Seq. ID No. 832, Seq. ID No. 836, Seq. ID No. 838, Seq. ID No. 840, Seq. ID No. 848, Seq. ID No. 852, Seq. ID No. 856, Seq. ID No. 860, Seq. ID No. 868, Seq. ID No. 872, Seq. ID No. 876, Seq. ID No. 882, Seq. ID No. 886, Seq. ID No. 890, Seq. ID No. 892, Seq. ID No. 896, Seq. ID No. 902, Seq. ID No. 908, Seq. ID No. 916, Seq. ID No. 920, Seq. ID No. 926, Seq. ID No. 930, Seq. ID No. 936, Seq. ID No. 940, Seq. ID No. 950, Seq. ID No. 952, Seq. ID No. 956, Seq. ID No. 962, Seq. ID No. 966, Seq. ID No. 968, Seq. ID No. 972, Seq. ID No. 986, Seq. ID No. 994, Seq. ID No. 998, Seq. ID No. 1004, Seq. ID No. 1006, Seq. ID No. 1010, Seq. ID No. 1018, Seq. ID No. 1024, Seq. ID No. 1040, Seq. ID No. 1042, Seq. ID No. 1044, Seq. ID No. 1052, Seq. ID No. 1054, Seq. ID No. 1058, Seq. ID No. 1062, Seq. ID No. 1066, Seq. ID No. 1068, Seq. ID No. 1070, Seq. ID No. 1074, Seq. ID No. 1076, Seq. ID No. 1084, Seq. ID No. 1088, Seq. ID No. 1092, Seq. ID No. 1094, Seq. ID No. 1098, Seq. ID No. 1100, Seq. ID No. 1104, Seq. ID No. 1110, Seq. ID No. 1116, Seq. ID No. 1118, Seq. ID No. 1124, Seq. ID No. 1130, Seq. ID No. 1134, Seq. ID No. 1138, Seq. ID No. 1144, Seq. ID No. 1148, Seq. ID No. 1150, Seq. ID No. 1156, Seq. ID No. 1158, Seq. ID No. 1160, Seq. ID No. 1164, Seq. ID No. 1172, Seq. ID No. 1180, Seq. ID No. 1182, Seq. ID No. 1188, Seq. ID No. 1194, Seq. ID No. 1200, Seq. ID No. 1206, Seq. ID No. 1212, Seq. ID No. 1218, Seq. ID No. 1222, Seq. ID No. 1224, Seq. ID No. 1226, Seq. ID No. 1232, Seq. ID No. 1234, Seq. ID No. 1238, Seq. ID No. 1242, Seq. ID No. 1254, Seq. ID No. 1258, Seq. ID No. 1264, Seq. ID No. 1272, Seq. ID No. 1282, Seq. ID No. 1318, Seq. ID No. 1324, Seq. ID No. 1332, Seq. ID No. 1336, Seq. ID No. 1340, Seq. ID No. 1346, Seq. ID No. 1348, Seq. ID No. 1362, Seq. ID No. 1370, Seq. ID No. 1378, Seq. ID No. 1380, Seq. ID No. 1388, Seq. ID No. 1394, Seq. ID No. 1396, Seq. ID No. 1400, Seq. ID No. 1404, Seq. ID No. 1406, Seq. ID No. 1412, Seq. ID No. 1414, Seq. ID No. 1416, Seq. ID No. 1420, Seq. ID No. 1426, Seq. ID No. 1428, Seq. ID No. 1430, Seq. ID No. 1432, Seq. ID No. 1434, Seq. ID No. 1438, Seq. ID No. 1444, Seq. ID No. 1450, Seq. ID No. 1452, Seq. ID No. 1456, Seq. ID No. 1458, Seq. ID No. 1460, Seq. ID No. 1464, Seq. ID No. 1466, Seq. ID No. 1470, Seq. ID No. 1472, Seq. ID No. 1474, Seq. ID No. 1476, Seq. ID No. 1478, Seq. ID No. 1486, Seq. ID No. 1488, Seq. ID No. 1492, Seq. ID No. 1494, Seq. ID No. 1496, Seq. ID No. 1498, Seq. ID No. 1502, Seq. ID No. 1506, Seq. ID No. 1508, Seq. ID No. 1510, Seq. ID No. 1518, Seq. ID No. 1526, Seq. ID No. 1528, Seq. ID No. 1532, Seq. ID No. 1540, Seq. ID No. 1542, Seq. ID No. 1544, Seq. ID No. 1550, Seq. ID No. 1562, Seq. ID No. 1564, Seq. ID No. 1568, Seq. ID No. 1572, Seq. ID No. 1574, Seq. ID No. 1578, Seq. ID No. 1582, Seq. ID No. 1590, Seq. ID No. 1594, Seq. ID No. 1596, Seq. ID No. 1598, Seq. ID No. 1604, Seq. ID No. 1608, Seq. ID No. 1612, Seq. ID No. 1614, Seq. ID No. 1620, Seq. ID No. 1624, Seq. ID No. 1628, Seq. ID No. 1632, Seq. ID No. 1638, Seq. ID No. 1640, Seq. ID No. 1642, Seq. ID No. 1648, Seq. ID No. 1654, Seq. ID No. 1658, Seq. ID No. 1666, Seq. ID No. 1670, Seq. ID No. 1682, Seq. ID No. 1686, Seq. ID No. 1690, Seq. ID No. 1692, Seq. ID No. 1698, Seq. ID No. 1700, Seq. ID No. 1708, Seq. ID No. 1712, Seq. ID No. 1714, Seq. ID No. 1720, Seq. ID No. 1724, Seq. ID No. 1730, Seq. ID No. 1736, Seq. ID No. 1738, Seq. ID No. 1742, Seq. ID No. 1748, Seq. ID No. 1752, Seq. ID No. 1758, Seq. ID No. 1760, Seq. ID No. 1762, Seq. ID No. 1772, Seq. ID No. 1778, Seq. ID No. 1786, Seq. ID No. 1790, Seq. ID No. 1792, Seq. ID No. 1798, Seq. ID No. 1802, Seq. ID No. 1804, Seq. ID No. 1806, Seq. ID No. 1812, Seq. ID No. 1818, Seq. ID No. 1820, Seq. ID No. 1822, Seq. ID No. 1826, Seq. ID No. 1830, Seq. ID No. 1838, Seq. ID No. 1844, Seq. ID No. 1846, Seq. ID No. 1848, Seq. ID No. 1850, Seq. ID No. 1852, Seq. ID No. 1856, Seq. ID No. 1860, Seq. ID No. 1864, Seq. ID No. 1866, Seq. ID No. 1868, Seq. ID No. 1872, Seq. ID No. 1874, Seq. ID No. 1878, Seq. ID No. 1886, Seq. ID No. 1888, Seq. ID No. 1890, Seq. ID No. 1896, Seq. ID No. 1898, Seq. ID No. 1900, Seq. ID No. 1904, Seq. ID No. 1906, Seq. ID No. 1914, Seq. ID No. 1920, Seq. ID No. 1924, Seq. ID No. 1926, Seq. ID No. 1932, Seq. ID No. 1936, Seq. ID No. 1938, Seq. ID No. 1944, Seq. ID No. 1946, Seq. ID No. 1950, Seq. ID No. 1954, Seq. ID No. 1958, Seq. ID No. 1960, Seq. ID No. 1962, Seq. ID No. 1966, Seq. ID No. 1968, Seq. ID No. 1972, Seq. ID No. 1976, Seq. ID No. 1980, Seq. ID No. 1984, Seq. ID No. 1988, Seq. ID No. 1990, Seq. ID No. 1992, Seq. ID No. 2000, Seq. ID No. 2002, Seq. ID No. 2004, Seq. ID No. 2010, Seq. ID No. 2016, Seq. ID No. 2020, Seq. ID No. 2022, Seq. ID No. 2032, Seq. ID No. 2036, Seq. ID No. 2038, Seq. ID No. 2046, Seq. ID No. 2048, Seq. ID No. 2056, Seq. ID No. 2060, Seq. ID No. 2066, Seq. ID No. 2068, Seq. ID No. 2070, Seq. ID No. 2072, Seq. ID No. 2074, Seq. ID No. 2080, Seq. ID No. 2092, Seq. ID No. 2094, Seq. ID No. 2098, Seq. ID No. 2100, Seq. ID No. 2104, Seq. ID No. 2116, Seq. ID No. 2120, Seq. ID No. 2128, Seq. ID No. 2136, Seq. ID No. 2138, Seq. ID No. 2142, Seq. ID No. 2144, Seq. ID No. 2148, Seq. ID No. 2154, Seq. ID No. 2160, Seq. ID No. 2162, Seq. ID No. 2166, Seq. ID No. 2170, Seq. ID No. 2180, Seq. ID No. 2184, Seq. ID No. 2186, Seq. ID No. 2190, Seq. ID No. 2192, Seq. ID No. 2194, Seq. ID No. 2196, Seq. ID No. 2200, Seq. ID No. 2208, Seq. ID No. 2218, Seq. ID No. 2220, Seq. ID No. 2222, Seq. ID No. 2224, Seq. ID No. 2228, Seq. ID No. 2230, Seq. ID No. 2232, Seq. ID No. 2234, Seq. ID No. 2242, Seq. ID No. 2246, Seq. ID No. 2252, Seq. ID No. 2254, Seq. ID No. 2258, Seq. ID No. 2260, Seq. ID No. 2264, Seq. ID No. 2268, Seq. ID No. 2270, Seq. ID No. 2276, Seq. ID No. 2282, Seq. ID No. 2288, Seq. ID No. 2298, Seq. ID No. 2300, Seq. ID No. 2302, Seq. ID No. 2316, Seq. ID No. 2330, Seq. ID No. 2334, Seq. ID No. 2336, Seq. ID No. 2340, Seq. ID No. 2344, Seq. ID No. 2346, Seq. ID No. 2350, Seq. ID No. 2352, Seq. ID No. 2358, Seq. ID No. 2360, Seq. ID No. 2364, Seq. ID No. 2366, Seq. ID No. 2370, Seq. ID No. 2374, Seq. ID No. 2382, Seq. ID No. 2392, Seq. ID No. 2400, Seq. ID No. 2402, Seq. ID No. 2406, Seq. ID No. 2410, Seq. ID No. 2416, Seq. ID No. 2418, Seq. ID No. 2422, Seq. ID No. 2424, Seq. ID No. 2428, Seq. ID No. 2430, Seq. ID No. 2432, Seq. ID No. 2436, Seq. ID No. 2438, Seq. ID No. 2440, Seq. ID No. 2444, Seq. ID No. 2446, Seq. ID No. 2450, Seq. ID No. 2452, Seq. ID No. 2454, Seq. ID No. 2458, Seq. ID No. 2464, Seq. ID No. 2466, Seq. ID No. 2470, Seq. ID No. 2472, Seq. ID No. 2476, Seq. ID No. 2478, Seq. ID No. 2484, Seq. ID No. 2486, Seq. ID No. 2492, Seq. ID No. 2496, Seq. ID No. 2506, Seq. ID No. 2508, Seq. ID No. 2514, Seq. ID No. 2520, Seq. ID No. 2526, Seq. ID No. 2530, Seq. ID No. 2536, Seq. ID No. 2538, Seq. ID No. 2540, Seq. ID No. 2550, Seq. ID No. 2554, Seq. ID No. 2564, Seq. ID No. 2568, Seq. ID No. 2576, Seq. ID No. 2578, Seq. ID No. 2582, Seq. ID No. 2590, Seq. ID No. 2594, Seq. ID No. 2596, Seq. ID No. 2600, Seq. ID No. 2604, Seq. ID No. 2608, Seq. ID No. 2610, Seq. ID No. 2614, Seq. ID No. 2618, Seq. ID No. 2620, Seq. ID No. 2624, Seq. ID No. 2626, Seq. ID No. 2632, Seq. ID No. 2634, Seq. ID No. 2640, Seq. ID No. 2642, Seq. ID No. 2646, Seq. ID No. 2650, Seq. ID No. 2652, Seq. ID No. 2656, Seq. ID No. 2660, Seq. ID No. 2664, Seq. ID No. 2668, Seq. ID No. 2670, Seq. ID No. 2672, Seq. ID No. 2678, Seq. ID No. 2688, Seq. ID No. 2690, Seq. ID No. 2694, Seq. ID No. 2700, Seq. ID No. 2704, Seq. ID No. 2710, Seq. ID No. 2712, Seq. ID No. 2716, Seq. ID No. 2720, Seq. ID No. 2726, Seq. ID No. 2728, Seq. ID No. 2738, Seq. ID No. 2744, Seq. ID No. 2748, Seq. ID No. 2752, Seq. ID No. 2754, Seq. ID No. 2760, Seq. ID No. 2764, Seq. ID No. 2766, Seq. ID No. 2768, Seq. ID No. 2772, Seq. ID No. 2774, Seq. ID No. 2776, Seq. ID No. 2782, Seq. ID No. 2788, Seq. ID No. 2790, Seq. ID No. 2792, Seq. ID No. 2794, Seq. ID No. 2798, Seq. ID No. 2800, Seq. ID No. 2804, Seq. ID No. 2806, Seq. ID No. 2812, Seq. ID No. 2816, Seq. ID No. 2824, Seq. ID No. 2828, Seq. ID No. 2844, Seq. ID No. 2848, Seq. ID No. 2864, Seq. ID No. 2868, Seq. ID No. 2870, Seq. ID No. 2872, Seq. ID No. 2878, Seq. ID No. 2882, Seq. ID No. 2892, Seq. ID No. 2900, Seq. ID No. 2902, Seq. ID No. 2904, Seq. ID No. 2908, Seq. ID No. 2912, Seq. ID No. 2914, Seq. ID No. 2918, Seq. ID No. 2920, Seq. ID No. 2922, Seq. ID No. 2924, Seq. ID No. 2928, Seq. ID No. 2934, Seq. ID No. 2946, Seq. ID No. 2954, Seq. ID No. 2958, Seq. ID No. 2964, Seq. ID No. 2972, Seq. ID No. 2974, Seq. ID No. 2978, Seq. ID No. 2980, Seq. ID No. 2984, Seq. ID No. 2986, Seq. ID No. 2992, Seq. ID No. 2994, Seq. ID No. 3000, Seq. ID No. 3002, Seq. ID No. 3006, Seq. ID No. 3012, Seq. ID No. 3020, Seq. ID No. 3028, Seq. ID No. 3034, Seq. ID No. 3036, Seq. ID No. 3040, Seq. ID No. 3044, Seq. ID No. 3046, Seq. ID No. 3050, Seq. ID No. 3056, Seq. ID No. 3060, Seq. ID No. 3062, Seq. ID No. 3068, Seq. ID No. 3070, Seq. ID No. 3082, Seq. ID No. 3090, Seq. ID No. 3092, Seq. ID No. 3094, Seq. ID No. 3098, Seq. ID No. 3104, Seq. ID No. 3108, Seq. ID No. 3112, Seq. ID No. 3118, Seq. ID No. 3126, Seq. ID No. 3134, Seq. ID No. 3136, Seq. ID No. 3138, Seq. ID No. 3142, Seq. ID No. 3144, Seq. ID No. 3150, Seq. ID No. 3156, Seq. ID No. 3158, Seq. ID No. 3160, Seq. ID No. 3162, Seq. ID No. 3166, Seq. ID No. 3172, Seq. ID No. 3178, Seq. ID No. 3180, Seq. ID No. 3184, Seq. ID No. 3186, Seq. ID No. 3190, Seq. ID No. 3192, Seq. ID No. 3198, Seq. ID No. 3204, Seq. ID No. 3206, Seq. ID No. 3212, Seq. ID No. 3218, Seq. ID No. 3222, Seq. ID No. 3228, Seq. ID No. 3230, Seq. ID No. 3234, Seq. ID No. 3236, Seq. ID No. 3240, Seq. ID No. 3250, Seq. ID No. 3252, Seq. ID No. 3254, Seq. ID No. 3258, Seq. ID No. 3260, Seq. ID No. 3264, Seq. ID No. 3266, Seq. ID No. 3272, Seq. ID No. 3274, Seq. ID No. 3278, Seq. ID No. 3284, Seq. ID No. 3286, Seq. ID No. 3288, Seq. ID No. 3290, Seq. ID No. 3294, Seq. ID No. 3296, Seq. ID No. 3300, Seq. ID No. 3304, Seq. ID No. 3308, Seq. ID No. 3314, Seq. ID No. 3316, Seq. ID No. 3320, Seq. ID No. 3324, Seq. ID No. 3330, Seq. ID No. 3336, Seq. ID No. 3338, Seq. ID No. 3348, Seq. ID No. 3356, Seq. ID No. 3360, Seq. ID No. 3364, Seq. ID No. 3368, Seq. ID No. 3378, Seq. ID No. 3380, Seq. ID No. 3384, Seq. ID No. 3388, Seq. ID No. 3400, Seq. ID No. 3402, Seq. ID No. 3404, Seq. ID No. 3410, Seq. ID No. 3414, Seq. ID No. 3416, Seq. ID No. 3418, Seq. ID No. 3426, Seq. ID No. 3428, Seq. ID No. 3432, Seq. ID No. 3436, Seq. ID No. 3438, Seq. ID No. 3440, Seq. ID No. 3448, Seq. ID No. 3460, Seq. ID No. 3462, Seq. ID No. 3468, Seq. ID No. 3470, Seq. ID No. 3472, Seq. ID No. 3474, Seq. ID No. 3476, Seq. ID No. 3480, Seq. ID No. 3484, Seq. ID No. 3486, Seq. ID No. 3488, Seq. ID No. 3496, Seq. ID No. 3502, Seq. ID No. 3506, Seq. ID-No. 3508, Seq. ID No. 3510, Seq. ID No. 3512, Seq. ID No. 3520, Seq. ID No. 3522, Seq. ID No. 3526, Seq. ID No. 3530, Seq. ID No. 3534, Seq. ID No. 3538, Seq. ID No. 3544, Seq. ID No. 3550, Seq. ID No. 3554, Seq. ID No. 3562, Seq. ID No. 3566, Seq. ID No. 3570, Seq. ID No. 3576, Seq. ID No. 3580, Seq. ID No. 3584, Seq. ID No. 3586, Seq. ID No. 3590, Seq. ID No. 3600, Seq. ID No. 3606, Seq. ID No. 3608, Seq. ID No. 3610, Seq. ID No. 3612, Seq. ID No. 3616, Seq. ID No. 3620, Seq. ID No. 3630, Seq. ID No. 3636, Seq. ID No. 3640, Seq. ID No. 3642, Seq. ID No. 3644, Seq. ID No. 3646, Seq. ID No. 3652, Seq. ID No. 3654, Seq. ID No. 3660, Seq. ID No. 3664, Seq. ID No. 3666, Seq. ID No. 3668, Seq. ID No. 3670, Seq. ID No. 3676, Seq. ID No. 3682, Seq. ID No. 3690, Seq. ID No. 3696, Seq. ID No. 3700, Seq. ID No. 3710, Seq. ID No. 3716, Seq. ID No. 3722, Seq. ID No. 3728, Seq. ID No. 3730, Seq. ID No. 3734, Seq. ID No. 3736, Seq. ID No. 3740, Seq. ID No. 3742, Seq. ID No. 3748, Seq. ID No. 3752, Seq. ID No. 3760, Seq. ID No. 3772, Seq. ID No. 3776, Seq. ID No. 3780, Seq. ID No. 3784, Seq. ID No. 3792, Seq. ID No. 3798, Seq. ID No. 3804, Seq. ID No. 3806, Seq. ID No. 3812, Seq. ID No. 3814, Seq. ID No. 3820, Seq. ID No. 3824, Seq. ID No. 3826, Seq. ID No. 3834, Seq. ID No. 3836, Seq. ID No. 3844, Seq. ID No. 3848, Seq. ID No. 3856, Seq. ID No. 3858, Seq. ID No. 3864, Seq. ID No. 3868, Seq. ID No. 3874, Seq. ID No. 3878, Seq. ID No. 3882, Seq. ID No. 3884, Seq. ID No. 3886, Seq. ID No. 3894, Seq. ID No. 3898, Seq. ID No. 3902, Seq. ID No. 3908, Seq. ID No. 3914, Seq. ID No. 3916, Seq. ID No. 3918, Seq. ID No. 3920, Seq. ID No. 3926, Seq. ID No. 3930, Seq. ID No. 3934, Seq. ID No. 3936, Seq. ID No. 3938, Seq. ID No. 3942, Seq. ID No. 3944, Seq. ID No. 3946, Seq. ID No. 3950, Seq. ID No. 3952, Seq. ID No. 3956, Seq. ID No. 3958, Seq. ID No. 3972, Seq. ID No. 3976, Seq. ID No. 3984, Seq. ID No. 3986, Seq. ID No. 3988, Seq. ID No. 3992, Seq. ID No. 4004, Seq. ID No. 4010, Seq. ID No. 4014, Seq. ID No. 4016, Seq. ID No. 4018, Seq. ID No. 4022, Seq. ID No. 4024, Seq. ID No. 4030, Seq. ID No. 4032, Seq. ID No. 4036, Seq. ID No. 4038, Seq. ID No. 4040, Seq. ID No. 4050, Seq. ID No. 4056, Seq. ID No. 4058, Seq. ID No. 4062, Seq. ID No. 4064, Seq. ID No. 4068, Seq. ID No. 4070, Seq. ID No. 4076, Seq. ID No. 4078, Seq. ID No. 4080, Seq. ID No. 4088, Seq. ID No. 4090, Seq. ID No. 4096, Seq. ID No. 4098, Seq. ID No. 4100, Seq. ID No. 4106, Seq. ID No. 4112, Seq. ID No. 4118, Seq. ID No. 4122, Seq. ID No. 4124, Seq. ID No. 4128, Seq. ID No. 4130, Seq. ID No. 4134, Seq. ID No. 4138, Seq. ID No. 4146, Seq. ID No. 4150, Seq. ID No. 4158, Seq. ID No. 4162, Seq. ID No. 4164, Seq. ID No. 4168, Seq. ID No. 4174, Seq. ID No. 4180, Seq. ID-No. 4186, Seq. ID No. 4192, Seq. ID No. 4194, Seq. ID No. 4196, Seq. ID No. 4198, Seq. ID No. 4200, Seq. ID No. 4202, Seq. ID No. 4204, Seq. ID No. 4206, Seq. ID No. 4212, Seq. ID No. 4216, Seq. ID No. 4218, Seq. ID No. 4220, Seq. ID No. 4236, Seq. ID No. 4238, Seq. ID No. 4240, Seq. ID No. 4244, Seq. ID No. 4252, Seq. ID No. 4256, Seq. ID No. 4264, Seq. ID No. 4268, Seq. ID No. 4272, Seq. ID No. 4274, Seq. ID No. 4280, Seq. ID No. 4282, Seq. ID No. 4284, Seq. ID No. 4292, Seq. ID No. 4296, Seq. ID No. 4300, Seq. ID No. 4302, Seq. ID No. 4304, Seq. ID No. 4308, Seq. ID No. 4310, Seq. ID No. 4314, Seq. ID No. 4318, Seq. ID No. 4324, Seq. ID No. 4326, Seq. ID No. 4330, Seq. ID No. 4332, Seq. ID No. 4334, Seq. ID No. 4340, Seq. ID No. 4344, Seq. ID No. 4348, Seq. ID No. 4354, Seq. ID No. 4356, Seq. ID No. 4358, Seq. ID No. 4366, Seq. ID No. 4370, Seq. ID No. 4374, Seq. ID No. 4376, Seq. ID No. 4378, Seq. ID No. 4380, Seq. ID No. 4382, Seq. ID No. 4384, Seq. ID No. 4392, Seq. ID No. 4394, Seq. ID No. 4398, Seq. ID No. 4402, Seq. ID No. 4412, Seq. ID No. 4418, Seq. ID No. 4424, Seq. ID No. 4432, Seq. ID No. 4434, Seq. ID No. 4436, Seq. ID No. 4444, Seq. ID No. 4448, Seq. ID No. 4452, Seq. ID No. 4460, Seq. ID No. 4462, Seq. ID No. 4468, Seq. ID No. 4472, Seq. ID No. 4476, Seq. ID No. 4480, Seq. ID No. 4482, Seq. ID No. 4488, Seq. ID No. 4492, Seq. ID No. 4502, Seq. ID No. 4506, Seq. ID No. 4512, Seq. ID No. 4524, Seq. ID No. 4532, Seq. ID No. 4534, Seq. ID No. 4538, Seq. ID No. 4540, Seq. ID No. 4542, Seq. ID No. 4544, Seq. ID No. 4548, Seq. ID No. 4550, Seq. ID No. 4560, Seq. ID No. 4566, Seq. ID No. 4568, Seq. ID No. 4572, Seq. ID No. 4576, Seq. ID No. 4578, Seq. ID No. 4582, Seq. ID No. 4586, Seq. ID No. 4592, Seq. ID No. 4596, Seq. ID No. 4598, Seq. ID No. 4604, Seq. ID No. 4608, Seq. ID No. 4610, Seq. ID No. 4612, Seq. ID No. 4616, Seq. ID No. 4624, Seq. ID No. 4628, Seq. ID No. 4632, Seq. ID No. 4636, Seq. ID No. 4638, Seq. ID No. 4644, Seq. ID No. 4650, Seq. ID No. 4652, Seq. ID No. 4654, Seq. ID No. 4658, Seq. ID No. 4662, Seq. ID No. 4664, Seq. ID No. 4670, Seq. ID No. 4672, Seq. ID No. 4674, Seq. ID No. 4680, Seq. ID No. 4686, Seq. ID No. 4694, Seq. ID No. 4698, Seq. ID No. 4704, Seq. ID No. 4706, Seq. ID No. 4714, Seq. ID No. 4718, Seq. ID No. 4720, Seq. ID No. 4726, Seq. ID No. 4730, Seq. ID No. 4732, Seq. ID No. 4734, Seq. ID No. 4740, Seq. ID No. 4744, Seq. ID No. 4746, Seq. ID No. 4748, Seq. ID No. 4750, Seq. ID No. 4752, Seq. ID No. 4758, Seq. ID No. 4766, Seq. ID No. 4770, Seq. ID No. 4778, Seq. ID No. 4780, Seq. ID No. 4782, Seq. ID No. 4788, Seq. ID No. 4790, Seq. ID No. 4792, Seq. ID No. 4798, Seq. ID No. 4804, Seq. ID No. 4808, Seq. ID No. 4810, Seq. ID No. 4812, Seq. ID No. 4814, Seq. ID No. 4816, Seq. ID No. 4822, Seq. ID No. 4824, Seq. ID No. 4826, Seq. ID No. 4828, Seq. ID No. 4834, Seq. ID No. 4836, Seq. ID No. 4838, Seq. ID No. 4844, Seq. ID No. 4846, Seq. ID No. 4848, Seq. ID No. 4850, Seq. ID No. 4860, Seq. ID No. 4862, Seq. ID No. 4866, Seq. ID No. 4868, Seq. ID No. 4876, Seq. ID No. 4878, Seq. ID No. 4880, Seq. ID No. 4882, Seq. ID No. 4884, Seq. ID No. 4886, Seq. ID No. 4888, Seq. ID No. 4894, Seq. ID No. 4900, Seq. ID No. 4902, Seq. ID No. 4904, Seq. ID No. 4906, Seq. ID No. 4908, Seq. ID No. 4912, Seq. ID No. 4916, Seq. ID No. 4918, Seq. ID No. 4920, Seq. ID No. 4922, Seq. ID No. 4928, Seq. ID No. 4932, Seq. ID No. 4936, Seq. ID No. 4940, Seq. ID No. 4942, Seq. ID No. 4944, Seq. ID No. 4946, Seq. ID No. 4948, Seq. ID No. 4954, Seq. ID No. 4958, Seq. ID No. 4960, Seq. ID No. 4964, Seq. ID No. 4966, Seq. ID No. 4970, Seq. ID No. 4972, Seq. ID No. 4974, Seq. ID No. 4980, Seq. ID No. 4986, Seq. ID No. 4990, Seq. ID No. 4992, Seq. ID No. 4996, Seq. ID No. 4998, Seq. ID No. 5002, Seq. ID No. 5008, Seq. ID No. 5010, Seq. ID No. 5018, Seq. ID No. 5022, Seq. ID No. 5028, Seq. ID No. 5030, Seq. ID No. 5040, Seq. ID No. 5048, Seq. ID No. 5052, Seq. ID No. 5054, Seq. ID No. 5056, Seq. ID No. 5060, Seq. ID No. 5062, Seq. ID No. 5070, Seq. ID No. 5078, Seq. ID No. 5080, Seq. ID No. 5082, Seq. ID No. 5084, Seq. ID No. 5088, Seq. ID No. 5092, Seq. ID No. 5094, Seq. ID No. 5098, Seq. ID No. 5104, Seq. ID No. 5106, Seq. ID No. 5110, Seq. ID No. 5112, Seq. ID No. 5114, Seq. ID No. 5116, Seq. ID No. 5118, Seq. ID No. 5128, Seq. ID No. 5142, Seq. ID No. 5146, Seq. ID No. 5150, Seq. ID No. 5154, Seq. ID No. 5158, Seq. ID No. 5166, Seq. ID No. 5170, Seq. ID No. 5172, Seq. ID No. 5178, Seq. ID No. 5184, Seq. ID No. 5186, Seq. ID No. 5188, Seq. ID No. 5194, Seq. ID No. 5196, Seq. ID No. 5202, Seq. ID No. 5204, Seq. ID No. 5206, Seq. ID No. 5210, Seq. ID No. 5212, Seq. ID No. 5216, Seq. ID No. 5220, Seq. ID No. 5228, Seq. ID No. 5232, Seq. ID No. 5236, Seq. ID No. 5238, Seq. ID No. 5240, Seq. ID No. 5244, Seq. ID No. 5246, Seq. ID No. 5248, Seq. ID No. 5254, Seq. ID No. 5256, Seq. ID No. 5258, Seq. ID No. 5262, Seq. ID No. 5264, Seq. ID No. 5272, Seq. ID No. 5276, Seq. ID No. 5284, Seq. ID No. 5292, Seq. ID No. 5300, Seq. ID No. 5306, Seq. ID No. 5310, Seq. ID No. 5312, Seq. ID No. 5314, Seq. ID No. 5320, Seq. ID No. 5324, Seq. ID No. 5326, Seq. ID No. 5336, Seq. ID No. 5338, Seq. ID No. 5342, Seq. ID No. 5346, Seq. ID No. 5352, Seq. ID No. 5356, Seq. ID No. 5364, Seq. ID No. 5366, Seq. ID No. 5368, Seq. ID No. 5372, Seq. ID No. 5374, Seq. ID No. 5382, Seq. ID No. 5384, Seq. ID No. 5388, Seq. ID No. 5390, Seq. ID No. 5392, Seq. ID No. 5394, Seq. ID No. 5396, Seq. ID No. 5398, Seq. ID No. 5400, Seq. ID No. 5402, Seq. ID No. 5404, Seq. ID No. 5410, Seq. ID No. 5412, Seq. ID No. 5416, Seq. ID No. 5418, Seq. ID No. 5420, Seq. ID No. 5422, Seq. ID No. 5424, Seq. ID No. 5428, Seq. ID No. 5432, Seq. ID No. 5434, Seq. ID No. 5440, Seq. ID No. 5442, Seq. ID No. 5444, Seq. ID No. 5448, Seq. ID No. 5450, Seq. ID No. 5452, Seq. ID No. 5454, Seq. ID No. 5456, Seq. ID No. 5458, Seq. ID No. 5474, Seq. ID No. 5478, Seq. ID No. 5484, Seq. ID No. 5488, Seq. ID No. 5492, Seq. ID No. 5496, Seq. ID No. 5500, Seq. ID No. 5504, Seq. ID No. 5508, Seq. ID No. 5510, Seq. ID No. 5514, Seq. ID No. 5528, Seq. ID No. 5532, Seq. ID No. 5538, Seq. ID No. 5540, Seq. ID No. 5542, Seq. ID No. 5548, Seq. ID No. 5554, Seq. ID No. 5558, Seq. ID No. 5560, Seq. ID No. 5564, Seq. ID No. 5568, Seq. ID No. 5572, Seq. ID No. 5576, Seq. ID No. 5582, Seq. ID No. 5586, Seq. ID No. 5592, Seq. ID No. 5600, Seq. ID No. 5602, Seq. ID No. 5606, Seq. ID No. 5616, Seq. ID No. 5622, Seq. ID No. 5630, Seq. ID No. 5632, Seq. ID No. 5638, Seq. ID No. 5648, Seq. ID No. 5652, Seq. ID No. 5656, Seq. ID No. 5658, Seq. ID No. 5662, Seq. ID No. 5666, Seq. ID No. 5670, Seq. ID No. 5672, Seq. ID No. 5674, Seq. ID No. 5680, Seq. ID No. 5686, Seq. ID No. 5694, Seq. ID No. 5698, Seq. ID No. 5700, Seq. ID No. 5702, Seq. ID No. 5708, Seq. ID No. 5716, Seq. ID No. 5718, Seq. ID No. 5724, Seq. ID No. 5728, Seq. ID No. 5736, Seq. ID No. 5740, Seq. ID No. 5750, Seq. ID No. 5752, Seq. ID No. 5756, Seq. ID No. 5762, Seq. ID No. 5772, Seq. ID No. 5776, Seq. ID No. 5778, Seq. ID No. 5780, Seq. ID No. 5784, Seq. ID No. 5790, Seq. ID No. 5796, Seq. ID No. 5802, Seq. ID No. 5804, Seq. ID No. 5810, Seq. ID No. 5816, Seq. ID No. 5820, Seq. ID No. 5826, Seq. ID No. 5834, Seq. ID No. 5836, Seq. ID No. 5842, Seq. ID No. 5846, Seq. ID No. 5848, Seq. ID No. 5850, Seq. ID No. 5852, Seq. ID No. 5854, Seq. ID No. 5856, Seq. ID No. 5860, Seq. ID No. 5866, Seq. ID No. 5872, Seq. ID No. 5876, Seq. ID No. 5878, Seq. ID No. 5882, Seq. ID No. 5888, Seq. ID No. 5892, Seq. ID No. 5896, Seq. ID No. 5898, Seq. ID No. 5902, Seq. ID No. 5904, Seq. ID No. 5912, Seq. ID No. 5914, Seq. ID No. 5916, Seq. ID No. 5918, Seq. ID No. 5920, Seq. ID No. 5930, Seq. ID No. 5932, Seq. ID No. 5934, Seq. ID No. 5938, Seq. ID No. 5942, Seq. ID No. 5944, Seq. ID No. 5948, Seq. ID No. 5952, Seq. ID No. 5954, Seq. ID No. 5956, Seq. ID No. 5958, Seq. ID No. 5962, Seq. ID No. 5970, Seq. ID No. 5974, Seq. ID No. 5980, Seq. ID No. 5982, Seq. ID No. 5986, Seq. ID No. 5990, Seq. ID No. 5994, Seq. ID No. 6002, Seq. ID No. 6004, Seq. ID No. 6012, Seq. ID No. 6016, Seq. ID No. 6018, Seq. ID No. 6022, Seq. ID No. 6028, Seq. ID No. 6030, Seq. ID No. 6034, Seq. ID No. 6036, Seq. ID No. 6042, Seq. ID No. 6044, Seq. ID No. 6054, Seq. ID No. 6058, Seq. ID No. 6066, Seq. ID No. 6072, Seq. ID No. 6080, Seq. ID No. 6086, Seq. ID No. 6090, Seq. ID No. 6096, Seq. ID No. 6098, Seq. ID No. 6102, Seq. ID No. 6104, Seq. ID No. 6112, Seq. ID No. 6114, Seq. ID No. 6118, Seq. ID No. 6124, Seq. ID No. 6126, Seq. ID No. 6130, Seq. ID No. 6132, Seq. ID No. 6134, Seq. ID No. 6142, Seq. ID No. 6146, Seq. ID No. 6150, Seq. ID No. 6158, Seq. ID No. 6166, Seq. ID No. 6170, Seq. ID No. 6172, Seq. ID No. 6176, Seq. ID No. 6178, Seq. ID No. 6182, Seq. ID No. 6188, Seq. ID No. 6196, Seq. ID No. 6202, Seq. ID No. 6210, Seq. ID No. 6214, Seq. ID No. 6216, Seq. ID No. 6220, Seq. ID No. 6234, Seq. ID No. 6236, Seq. ID No. 6244, Seq. ID No. 6250, Seq. ID No. 6252, Seq. ID No. 6258, Seq. ID No. 6262, Seq. ID No. 6264, Seq. ID No. 6272, Seq. ID No. 6276, Seq. ID No. 6278, Seq. ID No. 6282, Seq. ID No. 6288, Seq. ID No. 6298, Seq. ID No. 6300, Seq. ID No. 6306, Seq. ID No. 6312, Seq. ID No. 6316, Seq. ID No. 6322, Seq. ID No. 6324, Seq. ID No. 6328, Seq. ID No. 6332, Seq. ID No. 6334, Seq. ID No. 6338, Seq. ID No. 6340, Seq. ID No. 6342, Seq. ID No. 6344, Seq. ID No. 6346, Seq. ID No. 6354, Seq. ID No. 6358, Seq. ID No. 6360, Seq. ID No. 6362, Seq. ID No. 6368, Seq. ID No. 6370, Seq. ID No. 6374, Seq. ID No. 6376, Seq. ID No. 6378, Seq. ID No. 6380, Seq. ID No. 6390, Seq. ID No. 6396, Seq. ID No. 6400, Seq. ID No. 6404, Seq. ID No. 6408, Seq. ID No. 6412, Seq. ID No. 6414, Seq. ID No. 6418, Seq. ID No. 6424, Seq. ID No. 6434, Seq. ID No. 6438, Seq. ID No. 6440, Seq. ID No. 6446, Seq. ID No. 6450, Seq. ID No. 6452, Seq. ID No. 6458, Seq. ID No. 6460, Seq. ID No. 6462, Seq. ID No. 6464, Seq. ID No. 6478, Seq. ID No. 6482, Seq. ID No. 6484, Seq. ID No. 6488, Seq. ID No. 6490, Seq. ID No. 6492, Seq. ID No. 6494, Seq. ID No. 6496, Seq. ID No. 6504, Seq. ID No. 6506, Seq. ID No. 6512, Seq. ID No. 6516, Seq. ID No. 6520, Seq. ID No. 6524, Seq. ID No. 6526, Seq. ID No. 6530, Seq. ID No. 6532, Seq. ID No. 6534, Seq. ID No. 6538, Seq. ID No. 6546, Seq. ID No. 6552, Seq. ID No. 6554, Seq. ID No. 6560, Seq. ID No. 6564, Seq. ID No. 6568, Seq. ID No. 6578, Seq. ID No. 6580, Seq. ID No. 6584, Seq. ID No. 6586, Seq. ID No. 6588, Seq. ID No. 6590, Seq. ID No. 6604, Seq. ID No. 6606, Seq. ID No. 6610, Seq. ID No. 6614, Seq. ID No. 6616, Seq. ID No. 6618, Seq. ID No. 6622, Seq. ID No. 6624, Seq. ID No. 6628, Seq. ID No. 6630, Seq. ID No. 6634, and Seq. ID No. 6640.

In yet another preferred embodiment, the polypeptide is selected from the Alloiococcus otitidis polypeptides identified by GeneMark™ ORF finder program, and is selected from one of Seq. ID No. 2, Seq. ID No. 4, Seq. ID No. 12, Seq. ID No. 14, Seq. ID No. 16, Seq. ID No. 18, Seq. ID No. 26, Seq. ID No. 28, Seq. ID No. 30, Seq. ID No. 36, Seq. ID No. 38, Seq. ID No. 40, Seq. ID No. 42, Seq. ID No. 44, Seq. ID No. 46, Seq. ID No. 50, Seq. ID No. 54, Seq. ID No. 58, Seq. ID No. 62, Seq. ID No. 70, Seq. ID No. 72, Seq. ID No. 76, Seq. ID No. 82, Seq. ID No. 84, Seq. ID No. 90, Seq. ID No. 96, Seq. ID No. 102, Seq. ID No. 112, Seq. ID No. 120, Seq. ID No. 126, Seq. ID No. 130, Seq. ID No. 136, Seq. ID No. 138, Seq. ID No. 142, Seq. ID No. 148, Seq. ID No. 152, Seq. ID No. 156, Seq. ID No. 168, Seq. ID No. 172, Seq. ID No. 182, Seq. ID No. 186, Seq. ID No. 188, Seq. ID No. 190, Seq. ID No. 192, Seq. ID No. 196, Seq. ID No. 200, Seq. ID No. 208, Seq. ID No. 214, Seq. ID No. 220, Seq. ID No. 222, Seq. ID No. 226, Seq. ID No. 228, Seq. ID No. 230, Seq. ID No. 234, Seq. ID No. 240, Seq. ID No. 242, Seq. ID No. 244, Seq. ID No. 254, Seq. ID No. 262, Seq. ID No. 264, Seq. ID No. 268, Seq. ID No. 274, Seq. ID No. 276, Seq. ID No. 278, Seq. ID No. 280, Seq. ID No. 292, Seq. ID No. 294, Seq. ID No. 296, Seq. ID No. 300, Seq. ID No. 304, Seq. ID No. 310, Seq. ID No. 312, Seq. ID No. 316, Seq. ID No. 320, Seq. ID No. 324, Seq. ID No. 332, Seq. ID No. 342, Seq. ID No. 346, Seq. ID No. 352, Seq. ID No. 356, Seq. ID No. 362, Seq. ID No. 364, Seq. ID No. 366, Seq. ID No. 376, Seq. ID No. 380, Seq. ID No. 382, Seq. ID No. 384, Seq. ID No. 390, Seq. ID No. 392, Seq. ID No. 396, Seq. ID No. 400, Seq. ID No. 406, Seq. ID No. 410, Seq. ID No. 414, Seq. ID No. 418, Seq. ID No. 422, Seq. ID No. 424, Seq. ID No. 426, Seq. ID No. 428, Seq. ID No. 430, Seq. ID No. 436, Seq. ID No. 438, Seq. ID No. 440, Seq. ID No. 442, Seq. ID No. 444, Seq. ID No. 446, Seq. ID No. 448, Seq. ID No. 450, Seq. ID No. 460, Seq. ID No. 466, Seq. ID No. 486, Seq. ID No. 502, Seq. ID No. 506, Seq. ID No. 512, Seq. ID No. 514, Seq. ID No. 518, Seq. ID No. 520, Seq. ID No. 524, Seq. ID No. 530, Seq. ID No. 534, Seq. ID No. 536, Seq. ID No. 542, Seq. ID No. 544, Seq. ID No. 552, Seq. ID No. 554, Seq. ID No. 560, Seq. ID No. 564, Seq. ID No. 570, Seq. ID No. 576, Seq. ID No. 580, Seq. ID No. 584, Seq. ID No. 592, Seq. ID No. 596, Seq. ID No. 602, Seq. ID No. 606, Seq. ID No. 608, Seq. ID No. 612, Seq. ID No. 614, Seq. ID No. 616, Seq. ID No. 622, Seq. ID No. 628, Seq. ID No. 630, Seq. ID No. 634, Seq. ID No. 636, Seq. ID No. 638, Seq. ID No. 644, Seq. ID No. 650, Seq. ID No. 652, Seq. ID No. 656, Seq. ID No. 658, Seq. ID No. 670, Seq. ID No. 672, Seq. ID No. 676, Seq. ID No. 682, Seq. ID No. 684, Seq. ID No. 686, Seq. ID No. 694, Seq. ID No. 704, Seq. ID No. 706, Seq. ID No. 712, Seq. ID No. 726, Seq. ID No. 732, Seq. ID No. 736, Seq. ID No. 738, Seq. ID No. 742, Seq. ID No. 744, Seq. ID No. 750, Seq. ID No. 754, Seq. ID No. 760, Seq. ID No. 774, Seq. ID No. 782, Seq. ID No. 784, Seq. ID No. 786, Seq. ID No. 790, Seq. ID No. 794, Seq. ID No. 798, Seq. ID No. 804, Seq. ID No. 816, Seq. ID No. 820, Seq. ID No. 822, Seq. ID No. 828, Seq. ID No. 832, Seq. ID No. 834, Seq. ID No. 836, Seq. ID No. 838, Seq. ID No. 842, Seq. ID No. 844, Seq. ID No. 850, Seq. ID No. 852, Seq. ID No. 854, Seq. ID No. 858, Seq. ID No. 866, Seq. ID No. 870, Seq. ID No. 874, Seq. ID No. 878, Seq. ID No. 884, Seq. ID No. 888, Seq. ID No. 892, Seq. ID No. 896, Seq. ID No. 902, Seq. ID No. 908, Seq. ID No. 912, Seq. ID No. 918, Seq. ID No. 922, Seq. ID No. 928, Seq. ID No. 936, Seq. ID No. 940, Seq. ID No. 946, Seq. ID No. 952, Seq. ID No. 954, Seq. ID No. 960, Seq. ID No. 964, Seq. ID No. 968, Seq. ID No. 970, Seq. ID No. 978, Seq. ID No. 990, Seq. ID No. 996, Seq. ID No. 1000, Seq. ID No. 1006, Seq. ID No. 1008, Seq. ID No. 1016, Seq. ID No. 1022, Seq. ID No. 1036, Seq. ID No. 1042, Seq. ID No. 1044, Seq. ID No. 1050, Seq. ID No. 1054, Seq. ID No. 1058, Seq. ID No. 1060, Seq. ID No. 1064, Seq. ID No. 1068, Seq. ID No. 1070, Seq. ID No. 1072, Seq. ID No. 1076, Seq. ID No. 1084, Seq. ID No. 1086, Seq. ID No. 1090, Seq. ID No. 1094, Seq. ID No. 1098, Seq. ID No. 1100, Seq. ID No. 1102, Seq. ID No. 1110, Seq. ID No. 1114, Seq. ID No. 1118, Seq. ID No. 1122, Seq. ID No. 1130, Seq. ID No. 1134, Seq. ID No. 1138, Seq. ID No. 1142, Seq. ID No. 1146, Seq. ID No. 1150, Seq. ID No. 1156, Seq. ID No. 1158, Seq. ID No. 1160, Seq. ID No. 1164, Seq. ID No. 1172, Seq. ID No. 1174, Seq. ID No. 1176, Seq. ID No. 1182, Seq. ID No. 1186, Seq. ID No. 1190, Seq. ID No. 1200, Seq. ID No. 1206, Seq. ID No. 1210, Seq. ID No. 1216, Seq. ID No. 1220, Seq. ID No. 1224, Seq. ID No. 1226, Seq. ID No. 1234, Seq. ID No. 1236, Seq. ID No. 1242, Seq. ID No. 1252, Seq. ID No. 1256, Seq. ID No. 1262, Seq. ID No. 1272, Seq. ID No. 1282, Seq. ID No. 1316, Seq. ID No. 1324, Seq. ID No. 1332, Seq. ID No. 1334, Seq. ID No. 1340, Seq. ID No. 1346, Seq. ID No. 1348, Seq. ID No. 1356, Seq. ID No. 1358, Seq. ID No. 1370, Seq. ID No. 1374, Seq. ID No. 1388, Seq. ID No. 1392, Seq. ID No. 1396, Seq. ID No. 1398, Seq. ID No. 1404, Seq. ID No. 1406, Seq. ID No. 1408, Seq. ID No. 1414, Seq. ID No. 1416, Seq. ID No. 1418, Seq. ID No. 1426, Seq. ID No. 1428, Seq. ID No. 1430, Seq. ID No. 1432, Seq. ID No. 1434, Seq. ID No. 1436, Seq. ID No. 1440, Seq. ID No. 1442, Seq. ID No. 1452, Seq. ID No. 1454, Seq. ID No. 1460, Seq. ID No. 1462, Seq. ID No. 1470, Seq. ID No. 1472, Seq. ID No. 1474, Seq. ID No. 1476, Seq. ID No. 1478, Seq. ID No. 1484, Seq. ID No. 1488, Seq. ID No. 1490, Seq. ID No. 1494, Seq. ID No. 1496, Seq. ID No. 1498, Seq. ID No. 1502, Seq. ID No. 1504, Seq. ID No. 1508, Seq. ID No. 1510, Seq. ID No. 1518, Seq. ID No. 1522, Seq. ID No. 1528, Seq. ID No. 1532, Seq. ID No. 1538, Seq. ID No. 1542, Seq. ID No. 1544, Seq. ID No. 1550, Seq. ID No. 1562, Seq. ID No. 1564, Seq. ID No. 1566, Seq. ID No. 1570, Seq. ID No. 1574, Seq. ID No. 1576, Seq. ID No. 1582, Seq. ID No. 1588, Seq. ID No. 1592, Seq. ID No. 1596, Seq. ID No. 1598, Seq. ID No. 1606, Seq. ID No. 1610, Seq. ID No. 1614, Seq. ID No. 1620, Seq. ID No. 1624, Seq. ID No. 1628, Seq. ID No. 1630, Seq. ID No. 1636, Seq. ID No. 1642, Seq. ID No. 1648, Seq. ID No. 1652, Seq. ID No. 1658, Seq. ID No. 1660, Seq. ID No. 1668, Seq. ID No. 1670, Seq. ID No. 1680, Seq. ID No. 1684, Seq. ID No. 1688, Seq. ID No. 1692, Seq. ID No. 1698, Seq. ID No. 1700, Seq. ID No. 1702, Seq. ID No. 1708, Seq. ID No. 1710, Seq. ID No. 1714, Seq. ID No. 1718, Seq. ID No. 1722, Seq. ID No. 1726, Seq. ID No. 1736, Seq. ID No. 1738, Seq. ID No. 1740, Seq. ID No. 1744, Seq. ID No. 1746, Seq. ID No. 1750, Seq. ID No. 1756, Seq. ID No. 1770, Seq. ID No. 1774, Seq. ID No. 1778, Seq. ID No. 1786, Seq. ID No. 1790, Seq. ID No. 1792, Seq. ID No. 1798, Seq. ID No. 1800, Seq. ID No. 1804, Seq. ID No. 1806, Seq. ID No. 1808, Seq. ID No. 1818, Seq. ID No. 1820, Seq. ID No. 1822, Seq. ID No. 1826, Seq. ID No. 1828, Seq. ID No. 1832, Seq. ID No. 1836, Seq. ID No. 1842, Seq. ID No. 1846, Seq. ID No. 1848, Seq. ID No. 1850, Seq. ID No. 1852, Seq. ID No. 1856, Seq. ID No. 1860, Seq. ID No. 1862, Seq. ID No. 1866, Seq. ID No. 1868, Seq. ID No. 1872, Seq. ID No. 1874, Seq. ID No. 1878, Seq. ID No. 1880, Seq. ID No. 1888, Seq. ID No. 1890, Seq. ID No. 1896, Seq. ID No. 1898, Seq. ID No. 1900, Seq. ID No. 1902, Seq. ID No. 1906, Seq. ID No. 1910, Seq. ID No. 1918, Seq. ID No. 1922, Seq. ID No. 1926, Seq. ID No. 1932, Seq. ID No. 1934, Seq. ID No. 1938, Seq. ID No. 1940, Seq. ID No. 1946, Seq. ID No. 1948, Seq. ID No. 1952, Seq. ID No. 1956, Seq. ID No. 1960, Seq. ID No. 1962, Seq. ID No. 1966, Seq. ID No. 1968, Seq. ID No. 1970, Seq. ID No. 1974, Seq. ID No. 1978, Seq. ID No. 1980, Seq. ID No. 1984, Seq. ID No. 1990, Seq. ID No. 1992, Seq. ID No. 1998, Seq. ID No. 2002, Seq. ID No. 2004, Seq. ID No. 2012, Seq. ID No. 2022, Seq. ID No. 2024, Seq. ID No. 2030, Seq. ID No. 2034, Seq. ID No. 2038, Seq. ID No. 2040, Seq. ID No. 2046, Seq. ID No. 2048, Seq. ID No. 2052, Seq. ID No. 2058, Seq. ID No. 2062, Seq. ID No. 2068, Seq. ID No. 2070, Seq. ID No. 2072, Seq. ID No. 2074, Seq. ID No. 2078, Seq. ID No. 2090, Seq. ID No. 2094, Seq. ID No. 2096, Seq. ID No. 2100, Seq. ID No. 2102, Seq. ID No. 2114, Seq. ID No. 2118, Seq. ID No. 2124, Seq. ID No. 2136, Seq. ID No. 2138, Seq. ID No. 2140, Seq. ID No. 2144, Seq. ID No. 2148, Seq. ID No. 2154, Seq. ID No. 2160, Seq. ID No. 2164, Seq. ID No. 2168, Seq. ID No. 2176, Seq. ID No. 2182, Seq. ID No. 2186, Seq. ID No. 2190, Seq. ID No. 2194, Seq. ID No. 2196, Seq. ID No. 2198, Seq. ID No. 2206, Seq. ID No. 2218, Seq. ID No. 2220, Seq. ID No. 2224, Seq. ID No. 2226, Seq. ID No. 2230, Seq. ID No. 2236, Seq. ID No. 2240, Seq. ID No. 2244, Seq. ID No. 2246, Seq. ID No. 2248, Seq. ID No. 2254, Seq. ID No. 2256, Seq. ID No. 2258, Seq. ID No. 2260, Seq. ID No. 2262, Seq. ID No. 2266, Seq. ID No. 2270, Seq. ID No. 2274, Seq. ID No. 2286, Seq. ID No. 2298, Seq. ID No. 2300, Seq. ID No. 2302, Seq. ID No. 2314, Seq. ID No. 2328, Seq. ID No. 2332, Seq. ID No. 2336, Seq. ID No. 2338, Seq. ID No. 2342, Seq. ID No. 2344, Seq. ID No. 2346, Seq. ID No. 2350, Seq. ID No. 2352, Seq. ID No. 2358, Seq. ID No. 2360, Seq. ID No. 2362, Seq. ID No. 2366, Seq. ID No. 2370, Seq. ID No. 2374, Seq. ID No. 2378, Seq. ID No. 2384, Seq. ID No. 2398, Seq. ID No. 2402, Seq. ID No. 2404, Seq. ID No. 2410, Seq. ID No. 2412, Seq. ID No. 2418, Seq. ID No. 2422, Seq. ID No. 2424, Seq. ID No. 2426, Seq. ID No. 2430, Seq. ID No. 2436, Seq. ID No. 2438, Seq. ID No. 2440, Seq. ID No. 2442, Seq. ID No. 2446, Seq. ID No. 2450, Seq. ID No. 2452, Seq. ID No. 2454, Seq. ID No. 2462, Seq. ID No. 2466, Seq. ID No. 2468, Seq. ID No. 2472, Seq. ID No. 2474, Seq. ID No. 2478, Seq. ID No. 2480, Seq. ID No. 2482, Seq. ID No. 2486, Seq. ID No. 2488, Seq. ID No. 2496, Seq. ID No. 2506, Seq. ID No. 2508, Seq. ID No. 2510, Seq. ID No. 2520, Seq. ID No. 2526, Seq. ID No. 2528, Seq. ID No. 2530, Seq. ID No. 2536, Seq. ID No. 2538, Seq. ID No. 2540, Seq. ID No. 2548, Seq. ID No. 2552, Seq. ID No. 2564, Seq. ID No. 2566, Seq. ID No. 2574, Seq. ID No. 2578, Seq. ID No. 2580, Seq. ID No. 2586, Seq. ID No. 2594, Seq. ID No. 2596, Seq. ID No. 2600, Seq. ID No. 2608, Seq. ID No. 2610, Seq. ID No. 2614, Seq. ID No. 2616, Seq. ID No. 2620, Seq. ID No. 2624, Seq. ID No. 2626, Seq. ID No. 2632, Seq. ID No. 2634, Seq. ID No. 2640, Seq. ID No. 2642, Seq. ID No. 2644, Seq. ID No. 2648, Seq. ID No. 2652, Seq. ID No. 2656, Seq. ID No. 2658, Seq. ID No. 2662, Seq. ID No. 2666, Seq. ID No. 2670, Seq. ID No. 2672, Seq. ID No. 2674, Seq. ID No. 2682, Seq. ID No. 2690, Seq. ID No. 2694, Seq. ID No. 2700, Seq. ID No. 2704, Seq. ID No. 2710, Seq. ID No. 2712, Seq. ID No. 2714, Seq. ID No. 2720, Seq. ID No. 2722, Seq. ID No. 2728, Seq. ID No. 2738, Seq. ID No. 2742, Seq. ID No. 2746, Seq. ID No. 2750, Seq. ID No. 2754, Seq. ID No. 2758, Seq. ID No. 2762, Seq. ID No. 2766, Seq. ID No. 2768, Seq. ID No. 2770, Seq. ID No. 2774, Seq. ID No. 2776, Seq. ID No. 2778, Seq. ID No. 2788, Seq. ID No. 2790, Seq. ID No. 2792, Seq. ID No. 2794, Seq. ID No. 2796, Seq. ID No. 2800, Seq. ID No. 2804, Seq. ID No. 2816, Seq. ID No. 2818, Seq. ID No. 2820, Seq. ID No. 2826, Seq. ID No. 2844, Seq. ID No. 2848, Seq. ID No. 2850, Seq. ID No. 2852, Seq. ID No. 2858, Seq. ID No. 2860, Seq. ID No. 2864, Seq. ID No. 2868, Seq. ID No. 2870, Seq. ID No. 2872, Seq. ID No. 2878, Seq. ID No. 2880, Seq. ID No. 2890, Seq. ID No. 2892, Seq. ID No. 2898, Seq. ID No. 2902, Seq. ID No. 2904, Seq. ID No. 2908, Seq. ID No. 2910, Seq. ID No. 2914, Seq. ID No. 2916, Seq. ID No. 2920, Seq. ID No. 2922, Seq. ID No. 2924, Seq. ID No. 2926, Seq. ID No. 2930, Seq. ID No. 2936, Seq. ID No. 2944, Seq. ID No. 2952, Seq. ID No. 2956, Seq. ID No. 2964, Seq. ID No. 2966, Seq. ID No. 2972, Seq. ID No. 2974, Seq. ID No. 2976, Seq. ID No. 2980, Seq. ID No. 2982, Seq. ID No. 2986, Seq. ID No. 2990, Seq. ID No. 2994, Seq. ID No. 3000, Seq. ID No. 3002, Seq. ID No. 3004, Seq. ID No. 3018, Seq. ID No. 3028, Seq. ID No. 3034, Seq. ID No. 3036, Seq. ID No. 3040, Seq. ID No. 3042, Seq. ID No. 3044, Seq. ID No. 3046, Seq. ID No. 3048, Seq. ID No. 3056, Seq. ID No. 3058, Seq. ID No. 3062, Seq. ID No. 3066, Seq. ID No. 3070, Seq. ID No. 3080, Seq. ID No. 3090, Seq. ID No. 3092, Seq. ID No. 3094, Seq. ID No. 3096, Seq. ID No. 3104, Seq. ID No. 3106, Seq. ID No. 3110, Seq. ID No. 3114, Seq. ID No. 3126, Seq. ID No. 3128, Seq. ID No. 3136, Seq. ID No. 3138, Seq. ID No. 3142, Seq. ID No. 3144, Seq. ID No. 3148, Seq. ID No. 3156, Seq. ID No. 3158, Seq. ID No. 3160, Seq. ID No. 3162, Seq. ID No. 3166, Seq. ID No. 3170, Seq. ID No. 3176, Seq. ID No. 3180, Seq. ID No. 3184, Seq. ID No. 3186, Seq. ID No. 3190, Seq. ID No. 3192, Seq. ID No. 3196, Seq. ID No. 3200, Seq. ID No. 3206, Seq. ID No. 3212, Seq. ID No. 3218, Seq. ID No. 3222, Seq. ID No. 3226, Seq. ID No. 3230, Seq. ID No. 3232, Seq. ID No. 3238, Seq. ID No. 3240, Seq. ID No. 3248, Seq. ID No. 3252, Seq. ID No. 3254, Seq. ID No. 3256, Seq. ID No. 3260, Seq. ID No. 3262, Seq. ID No. 3268, Seq. ID No. 3274, Seq. ID No. 3278, Seq. ID No. 3280, Seq. ID No. 3286, Seq. ID No. 3288, Seq. ID No. 3290, Seq. ID No. 3294, Seq. ID No. 3296, Seq. ID No. 3300, Seq. ID No. 3302, Seq. ID No. 3306, Seq. ID No. 3310, Seq. ID No. 3316, Seq. ID No. 3320, Seq. ID No. 3322, Seq. ID No. 3328, Seq. ID No. 3334, Seq. ID No. 3338, Seq. ID No. 3344, Seq. ID No. 3354, Seq. ID No. 3360, Seq. ID No. 3362, Seq. ID No. 3366, Seq. ID No. 3374, Seq. ID No. 3382, Seq. ID No. 3386, Seq. ID No. 3394, Seq. ID No. 3402, Seq. ID No. 3408, Seq. ID No. 3412, Seq. ID No. 3416, Seq. ID No. 3418, Seq. ID No. 3424, Seq. ID No. 3430, Seq. ID No. 3438, Seq. ID No. 3440, Seq. ID No. 3444, Seq. ID No. 3460, Seq. ID No. 3462, Seq. ID No. 3466, Seq. ID No. 3470, Seq. ID No. 3472, Seq. ID No. 3474, Seq. ID No. 3476, Seq. ID No. 3478, Seq. ID No. 3484, Seq. ID No. 3488, Seq. ID No. 3496, Seq. ID No. 3498, Seq. ID No. 3506, Seq. ID No. 3508, Seq. ID No. 3510, Seq. ID No. 3512, Seq. ID No. 3516, Seq. ID No. 3522, Seq. ID No. 3524, Seq. ID No. 3530, Seq. ID No. 3536, Seq. ID No. 3542, Seq. ID No. 3552, Seq. ID No. 3560, Seq. ID No. 3564, Seq. ID No. 3568, Seq. ID No. 3576, Seq. ID No. 3578, Seq. ID No. 3584, Seq. ID No. 3586, Seq. ID No. 3590, Seq. ID No. 3592, Seq. ID No. 3600, Seq. ID No. 3602, Seq. ID No. 3608, Seq. ID No. 3610, Seq. ID No. 3612, Seq. ID No. 3616, Seq. ID No. 3620, Seq. ID No. 3630, Seq. ID No. 3632, Seq. ID No. 3634, Seq. ID No. 3638, Seq. ID No. 3642, Seq. ID No. 3644, Seq. ID No. 3646, Seq. ID No. 3652, Seq. ID No. 3654, Seq. ID No. 3656, Seq. ID No. 3662, Seq. ID No. 3666, Seq. ID No. 3668, Seq. ID No. 3670, Seq. ID No. 3676, Seq. ID No. 3682, Seq. ID No. 3690, Seq. ID No. 3696, Seq. ID No. 3698, Seq. ID No. 3706, Seq. ID No. 3712, Seq. ID No. 3720, Seq. ID No. 3726, Seq. ID No. 3730, Seq. ID No. 3732, Seq. ID No. 3736, Seq. ID No. 3740, Seq. ID No. 3742, Seq. ID No. 3744, Seq. ID No. 3750, Seq. ID No. 3760, Seq. ID No. 3770, Seq. ID No. 3774, Seq. ID No. 3778, Seq. ID No. 3782, Seq. ID No. 3788, Seq. ID No. 3796, Seq. ID No. 3802, Seq. ID No. 3806, Seq. ID No. 3810, Seq. ID No. 3814, Seq. ID No. 3820, Seq. ID No. 3822, Seq. ID No. 3826, Seq. ID No. 3828, Seq. ID No. 3830, Seq. ID No. 3832, Seq. ID No. 3836, Seq. ID No. 3842, Seq. ID No. 3846, Seq. ID No. 3856, Seq. ID No. 3858, Seq. ID No. 3864, Seq. ID No. 3868, Seq. ID No. 3870, Seq. ID No. 3876, Seq. ID No. 3882, Seq. ID No. 3884, Seq. ID No. 3886, Seq. ID No. 3888, Seq. ID No. 3898, Seq. ID No. 3902, Seq. ID No. 3906, Seq. ID No. 3910, Seq. ID No. 3914, Seq. ID No. 3916, Seq. ID No. 3918, Seq. ID No. 3920, Seq. ID No. 3922, Seq. ID No. 3930, Seq. ID No. 3932, Seq. ID No. 3936, Seq. ID No. 3942, Seq. ID No. 3944, Seq. ID No. 3946, Seq. ID No. 3948, Seq. ID No. 3952, Seq. ID No. 3954, Seq. ID No. 3958, Seq. ID No. 3972, Seq. ID No. 3978, Seq. ID No. 3982, Seq. ID No. 3986, Seq. ID No. 3988, Seq. ID No. 3990, Seq. ID No. 3994, Seq. ID No. 4002, Seq. ID No. 4006, Seq. ID No. 4012, Seq. ID No. 4016, Seq. ID No. 4018, Seq. ID No. 4020, Seq. ID No. 4024, Seq. ID No. 4030, Seq. ID No. 4032, Seq. ID No. 4034, Seq. ID No. 4038, Seq. ID No. 4040, Seq. ID No. 4050, Seq. ID No. 4052, Seq. ID No. 4058, Seq. ID No. 4060, Seq. ID No. 4066, Seq. ID No. 4070, Seq. ID No. 4076, Seq. ID No. 4080, Seq. ID No. 4086, Seq. ID No. 4090, Seq. ID No. 4096, Seq. ID No. 4098, Seq. ID No. 4100, Seq. ID No. 4106, Seq. ID No. 4112, Seq. ID No. 4116, Seq. ID No. 4122, Seq. ID No. 4124, Seq. ID No. 4128, Seq. ID No. 4130, Seq. ID No. 4132, Seq. ID No. 4138, Seq. ID No. 4142, Seq. ID No. 4148, Seq. ID No. 4158, Seq. ID No. 4160, Seq. ID No. 4164, Seq. ID No. 4166, Seq. ID No. 4170, Seq. ID No. 4180, Seq. ID No. 4184, Seq. ID No. 4188, Seq. ID No. 4194, Seq. ID No. 4196, Seq. ID No. 4198, Seq. ID No. 4200, Seq. ID No. 4202, Seq. ID No. 4206, Seq. ID No. 4208, Seq. ID No. 4214, Seq. ID No. 4220, Seq. ID No. 4236, Seq. ID No. 4238, Seq. ID No. 4240, Seq. ID No. 4244, Seq. ID No. 4246, Seq. ID No. 4256, Seq. ID No. 4260, Seq. ID No. 4268, Seq. ID No. 4272, Seq. ID No. 4278, Seq. ID No. 4280, Seq. ID No. 4282, Seq. ID No. 4284, Seq. ID No. 4290, Seq. ID No. 4294, Seq. ID No. 4300, Seq. ID No. 4302, Seq. ID No. 4304, Seq. ID No. 4306, Seq. ID No. 4308, Seq. ID No. 4310, Seq. ID No. 4314, Seq. ID No. 4316, Seq. ID No. 4324, Seq. ID No. 4326, Seq. ID No. 4328, Seq. ID No. 4332, Seq. ID No. 4334, Seq. ID No. 4338, Seq. ID No. 4342, Seq. ID No. 4346, Seq. ID No. 4350, Seq. ID No. 4356, Seq. ID No. 4358, Seq. ID No. 4366, Seq. ID No. 4368, Seq. ID No. 4372, Seq. ID No. 4376, Seq. ID No. 4378, Seq. ID No. 4380, Seq. ID No. 4384, Seq. ID No. 4388, Seq. ID No. 4394, Seq. ID No. 4398, Seq. ID No. 4402, Seq. ID No. 4410, Seq. ID No. 4414, Seq. ID No. 4422, Seq. ID No. 4434, Seq. ID No. 4436, Seq. ID No. 4446, Seq. ID No. 4450, Seq. ID No. 4458, Seq. ID No. 4462, Seq. ID No. 4464, Seq. ID No. 4472, Seq. ID No. 4476, Seq. ID No. 4478, Seq. ID No. 4482, Seq. ID No. 4488, Seq. ID No. 4492, Seq. ID No. 4500, Seq. ID No. 4502, Seq. ID No. 4504, Seq. ID No. 4510, Seq. ID No. 4518, Seq. ID No. 4532, Seq. ID No. 4534, Seq. ID No. 4536, Seq. ID No. 4540, Seq. ID No. 4542, Seq. ID No. 4546, Seq. ID No. 4550, Seq. ID No. 4558, Seq. ID No. 4564, Seq. ID No. 4568, Seq. ID No. 4570, Seq. ID No. 4576, Seq. ID No. 4578, Seq. ID No. 4580, Seq. ID No. 4584, Seq. ID No. 4592, Seq. ID No. 4594, Seq. ID No. 4598, Seq. ID No. 4602, Seq. ID No. 4606, Seq. ID No. 4610, Seq. ID No. 4612, Seq. ID No. 4614, Seq. ID No. 4624, Seq. ID No. 4626, Seq. ID No. 4630, Seq. ID No. 4634, Seq. ID No. 4638, Seq. ID No. 4642, Seq. ID No. 4646, Seq. ID No. 4652, Seq. ID No. 4654, Seq. ID No. 4656, Seq. ID No. 4662, Seq. ID No. 4664, Seq. ID No. 4670, Seq. ID No. 4672, Seq. ID No. 4674, Seq. ID No. 4676, Seq. ID No. 4686, Seq. ID No. 4690, Seq. ID No. 4698, Seq. ID No. 4704, Seq. ID No. 4706, Seq. ID No. 4714, Seq. ID No. 4718, Seq. ID No. 4720, Seq. ID No. 4724, Seq. ID No. 4730, Seq. ID No. 4732, Seq. ID No. 4734, Seq. ID No. 4740, Seq. ID No. 4744, Seq. ID No. 4746, Seq. ID No. 4748, Seq. ID No. 4750, Seq. ID No. 4752, Seq. ID No. 4754, Seq. ID No. 4762, Seq. ID No. 4768, Seq. ID No. 4772, Seq. ID No. 4780, Seq. ID No. 4782, Seq. ID No. 4784, Seq. ID No. 4786, Seq. ID No. 4790, Seq. ID No. 4792, Seq. ID No. 4794, Seq. ID No. 4800, Seq. ID No. 4808, Seq. ID No. 4810, Seq. ID No. 4812, Seq. ID No. 4814, Seq. ID No. 4816, Seq. ID No. 4822, Seq. ID No. 4826, Seq. ID No. 4828, Seq. ID No. 4830, Seq. ID No. 4838, Seq. ID No. 4840, Seq. ID No. 4846, Seq. ID No. 4848, Seq. ID No. 4850, Seq. ID No. 4858, Seq. ID No. 4862, Seq. ID No. 4866, Seq. ID No. 4868, Seq. ID No. 4876, Seq. ID No. 4880, Seq. ID No. 4882, Seq. ID No. 4884, Seq. ID No. 4886, Seq. ID No. 4888, Seq. ID No. 4894, Seq. ID No. 4900, Seq. ID No. 4902, Seq. ID No. 4904, Seq. ID No. 4906, Seq. ID No. 4908, Seq. ID No. 4910, Seq. ID No. 4916, Seq. ID No. 4918, Seq. ID No. 4920, Seq. ID No. 4922, Seq. ID No. 4926, Seq. ID No. 4930, Seq. ID No. 4934, Seq. ID No. 4938, Seq. ID No. 4942, Seq. ID No. 4944, Seq. ID No. 4946, Seq. ID No. 4948, Seq. ID No. 4954, Seq. ID No. 4956, Seq. ID No. 4960, Seq. ID No. 4962, Seq. ID No. 4966, Seq. ID No. 4968, Seq. ID No. 4972, Seq. ID No. 4974, Seq. ID No. 4978, Seq. ID No. 4986, Seq. ID No. 4990, Seq. ID No. 4992, Seq. ID No. 4996, Seq. ID No. 4998, Seq. ID No. 5006, Seq. ID No. 5010, Seq. ID No. 5016, Seq. ID No. 5020, Seq. ID No. 5026, Seq. ID No. 5030, Seq. ID No. 5038, Seq. ID No. 5046, Seq. ID No. 5050, Seq. ID No. 5054, Seq. ID No. 5056, Seq. ID No. 5060, Seq. ID No. 5062, Seq. ID No. 5066, Seq. ID No. 5072, Seq. ID No. 5074, Seq. ID No. 5080, Seq. ID No. 5082, Seq. ID No. 5084, Seq. ID No. 5088, Seq. ID No. 5090, Seq. ID No. 5094, Seq. ID No. 5096, Seq. ID No. 5104, Seq. ID No. 5106, Seq. ID No. 5108, Seq. ID No. 5112, Seq. ID No. 5114, Seq. ID No. 5116, Seq. ID No. 5118, Seq. ID No. 5120, Seq. ID No. 5142, Seq. ID No. 5144, Seq. ID No. 5148, Seq. ID No. 5154, Seq. ID No. 5156, Seq. ID No. 5164, Seq. ID No. 5170, Seq. ID No. 5172, Seq. ID No. 5174, Seq. ID No. 5180, Seq. ID No. 5186, Seq. ID No. 5188, Seq. ID No. 5194, Seq. ID No. 5196, Seq. ID No. 5202, Seq. ID No. 5204, Seq. ID No. 5206, Seq. ID No. 5210, Seq. ID No. 5212, Seq. ID No. 5214, Seq. ID No. 5218, Seq. ID No. 5226, Seq. ID No. 5232, Seq. ID No. 5234, Seq. ID No. 5238, Seq. ID No. 5240, Seq. ID No. 5246, Seq. ID No. 5248, Seq. ID No. 5250, Seq. ID No. 5256, Seq. ID No. 5258, Seq. ID No. 5260, Seq. ID No. 5264, Seq. ID No. 5270, Seq. ID No. 5274, Seq. ID No. 5284, Seq. ID No. 5290, Seq. ID No. 5294, Seq. ID No. 5308, Seq. ID No. 5312, Seq. ID No. 5314, Seq. ID No. 5316, Seq. ID No. 5324, Seq. ID No. 5326, Seq. ID No. 5336, Seq. ID No. 5338, Seq. ID No. 5340, Seq. ID No. 5346, Seq. ID No. 5348, Seq. ID No. 5350, Seq. ID No. 5354, Seq. ID No. 5362, Seq. ID No. 5366, Seq. ID No. 5368, Seq. ID No. 5370, Seq. ID No. 5374, Seq. ID No. 5376, Seq. ID No. 5388, Seq. ID No. 5390, Seq. ID No. 5392, Seq. ID No. 5394, Seq. ID No. 5396, Seq. ID No. 5398, Seq. ID No. 5400, Seq. ID No. 5402, Seq. ID No. 5404, Seq. ID No. 5406, Seq. ID No. 5412, Seq. ID No. 5416, Seq. ID No. 5418, Seq. ID No. 5420, Seq. ID No. 5422, Seq. ID No. 5424, Seq. ID No. 5430, Seq. ID No. 5434, Seq. ID No. 5442, Seq. ID No. 5444, Seq. ID No. 5446, Seq. ID No. 5450, Seq. ID No. 5452, Seq. ID No. 5454, Seq. ID No. 5456, Seq. ID No. 5458, Seq. ID No. 5470, Seq. ID No. 5476, Seq. ID No. 5484, Seq. ID No. 5486, Seq. ID No. 5490, Seq. ID No. 5494, Seq. ID No. 5500, Seq. ID No. 5502, Seq. ID No. 5506, Seq. ID No. 5510, Seq. ID No. 5512, Seq. ID No. 5524, Seq. ID No. 5530, Seq. ID No. 5536, Seq. ID No. 5540, Seq. ID No. 5542, Seq. ID No. 5548, Seq. ID No. 5552, Seq. ID No. 5556, Seq. ID No. 5560, Seq. ID No. 5562, Seq. ID No. 5566, Seq. ID No. 5572, Seq. ID No. 5574, Seq. ID No. 5580, Seq. ID No. 5584, Seq. ID No. 5590, Seq. ID No. 5600, Seq. ID No. 5602, Seq. ID No. 5604, Seq. ID No. 5614, Seq. ID No. 5622, Seq. ID No. 5628, Seq. ID No. 5632, Seq. ID No. 5636, Seq. ID No. 5646, Seq. ID No. 5652, Seq. ID No. 5654, Seq. ID No. 5658, Seq. ID No. 5662, Seq. ID No. 5664, Seq. ID No. 5666, Seq. ID No. 5670, Seq. ID No. 5672, Seq. ID No. 5674, Seq. ID No. 5676, Seq. ID No. 5682, Seq. ID No. 5694, Seq. ID No. 5696, Seq. ID No. 5700, Seq. ID No. 5702, Seq. ID No. 5708, Seq. ID No. 5714, Seq. ID No. 5718, Seq. ID No. 5724, Seq. ID No. 5726, Seq. ID No. 5732, Seq. ID No. 5738, Seq. ID No. 5748, Seq. ID No. 5752, Seq. ID No. 5756, Seq. ID No. 5760, Seq. ID No. 5772, Seq. ID No. 5774, Seq. ID No. 5778, Seq. ID No. 5780, Seq. ID No. 5782, Seq. ID No. 5786, Seq. ID No. 5794, Seq. ID No. 5800, Seq. ID No. 5806, Seq. ID No. 5812, Seq. ID No. 5816, Seq. ID No. 5818, Seq. ID No. 5824, Seq. ID No. 5832, Seq. ID No. 5836, Seq. ID No. 5840, Seq. ID No. 5844, Seq. ID No. 5848, Seq. ID No. 5850, Seq. ID No. 5852, Seq. ID No. 5854, Seq. ID No. 5856, Seq. ID No. 5858, Seq. ID No. 5862, Seq. ID No. 5868, Seq. ID No. 5876, Seq. ID No. 5878, Seq. ID No. 5882, Seq. ID No. 5890, Seq. ID No. 5896, Seq. ID No. 5898, Seq. ID No. 5902, Seq. ID No. 5904, Seq. ID No. 5908, Seq. ID No. 5914, Seq. ID No. 5916, Seq. ID No. 5918, Seq. ID No. 5920, Seq. ID No. 5926, Seq. ID No. 5932, Seq. ID No. 5934, Seq. ID No. 5936, Seq. ID No. 5942, Seq. ID No. 5944, Seq. ID No. 5946, Seq. ID No. 5950, Seq. ID No. 5956, Seq. ID No. 5958, Seq. ID No. 5960, Seq. ID No. 5968, Seq. ID No. 5972, Seq. ID No. 5976, Seq. ID No. 5978, Seq. ID No. 5982, Seq. ID No. 5984, Seq. ID No. 5986, Seq. ID No. 5990, Seq. ID No. 5992, Seq. ID No. 5996, Seq. ID No. 5998, Seq. ID No. 6004, Seq. ID No. 6006, Seq. ID No. 6008, Seq. ID No. 6016, Seq. ID No. 6018, Seq. ID No. 6020, Seq. ID No. 6024, Seq. ID No. 6030, Seq. ID No. 6034, Seq. ID No. 6036, Seq. ID No. 6038, Seq. ID No. 6044, Seq. ID No. 6050, Seq. ID No. 6056, Seq. ID No. 6066, Seq. ID No. 6070, Seq. ID No. 6076, Seq. ID No. 6082, Seq. ID No. 6084, Seq. ID No. 6088, Seq. ID No. 6096, Seq. ID No. 6098, Seq. ID No. 6100, Seq. ID No. 6104, Seq. ID No. 6106, Seq. ID No. 6114, Seq. ID No. 6116, Seq. ID No. 6120, Seq. ID No. 6126, Seq. ID No. 6128, Seq. ID No. 6130, Seq. ID No. 6132, Seq. ID No. 6134, Seq. ID No. 6142, Seq. ID No. 6144, Seq. ID No. 6148, Seq. ID No. 6154, Seq. ID No. 6166, Seq. ID No. 6170, Seq. ID No. 6172, Seq. ID No. 6174, Seq. ID No. 6178, Seq. ID No. 6180, Seq. ID No. 6188, Seq. ID No. 6194, Seq. ID No. 6202, Seq. ID No. 6208, Seq. ID No. 6212, Seq. ID No. 6216, Seq. ID No. 6220, Seq. ID No. 6232, Seq. ID No. 6236, Seq. ID No. 6244, Seq. ID No. 6248, Seq. ID No. 6252, Seq. ID No. 6256, Seq. ID No. 6262, Seq. ID No. 6264, Seq. ID No. 6270, Seq. ID No. 6276, Seq. ID No. 6278, Seq. ID No. 6280, Seq. ID No. 6288, Seq. ID No. 6292, Seq. ID No. 6300, Seq. ID No. 6306, Seq. ID No. 6308, Seq. ID No. 6314, Seq. ID No. 6316, Seq. ID No. 6322, Seq. ID No. 6324, Seq. ID No. 6326, Seq. ID No. 6332, Seq. ID No. 6336, Seq. ID No. 6340, Seq. ID No. 6342, Seq. ID No. 6344, Seq. ID No. 6346, Seq. ID No. 6348, Seq. ID No. 6356, Seq. ID No. 6360, Seq. ID No. 6362, Seq. ID No. 6366, Seq. ID No. 6370, Seq. ID No. 6372, Seq. ID No. 6376, Seq. ID No. 6378, Seq. ID No. 6380, Seq. ID No. 6388, Seq. ID No. 6392, Seq. ID No. 6398, Seq. ID No. 6402, Seq. ID No. 6406, Seq. ID No. 6410, Seq. ID No. 6414, Seq. ID No. 6418, Seq. ID No. 6420, Seq. ID No. 6424, Seq. ID No. 6430, Seq. ID No. 6434, Seq. ID No. 6436, Seq. ID No. 6440, Seq. ID No. 6446, Seq. ID No. 6448, Seq. ID No. 6452, Seq. ID No. 6456, Seq. ID No. 6460, Seq. ID No. 6462, Seq. ID No. 6464, Seq. ID No. 6476, Seq. ID No. 6482, Seq. ID No. 6484, Seq. ID No. 6486, Seq. ID No. 6488, Seq. ID No. 6490, Seq. ID No. 6492, Seq. ID No. 6494, Seq. ID No. 6496, Seq. ID No. 6500, Seq. ID No. 6506, Seq. ID No. 6510, Seq. ID No. 6514, Seq. ID No. 6520, Seq. ID No. 6522, Seq. ID No. 6526, Seq. ID No. 6528, Seq. ID No. 6532, Seq. ID No. 6534, Seq. ID No. 6536, Seq. ID No. 6546, Seq. ID No. 6552, Seq. ID No. 6554, Seq. ID No. 6560, Seq. ID No. 6562, Seq. ID No. 6564, Seq. ID No. 6566, Seq. ID No. 6568, Seq. ID No. 6570, Seq. ID No. 6576, Seq. ID No. 6580, Seq. ID No. 6584, Seq. ID No. 6586, Seq. ID No. 6592, Seq. ID No. 6596, Seq. ID No. 6606, Seq. ID No. 6608, Seq. ID No. 6612, Seq. ID No. 6616, Seq. ID No. 6618, Seq. ID No. 6620, Seq. ID No. 6624, Seq. ID No. 6626, Seq. ID No. 6630, Seq. ID No. 6634, Seq. ID No. 6636, Seq. ID No. 6642, and Seq. ID No. 6648.

In yet another preferred embodiment, the polypeptide is selected from the Alloiococcus otitidis polypeptides identified by the Applicants' assignee's ORF finder program that searches for an ATG, GTG or TTG Start codon between a Stop-Stop region, and wherein the polypeptide is selected from one Seq. ID No. 2, Seq. ID No. 6, Seq. ID No. 12, Seq. ID No. 14, Seq. ID No. 16, Seq. ID No. 18, Seq. ID No. 20, Seq. ID No. 22, Seq. ID No. 24, Seq. ID No. 28, Seq. ID No. 30, Seq. ID No. 32, Seq. ID No. 36, Seq. ID No. 38, Seq. ID No. 42, Seq. ID No. 44, Seq. ID No. 46, Seq. ID No. 48, Seq. ID No. 50, Seq. ID No. 52, Seq. ID No. 54, Seq. ID No. 56, Seq. ID No. 58, Seq. ID No. 60, Seq. ID No. 66, Seq. ID No. 68, Seq. ID No. 76, Seq. ID No. 78, Seq. ID No. 80, Seq. ID No. 82, Seq. ID No. 84, Seq. ID No. 90, Seq. ID No. 92, Seq. ID No. 94, Seq. ID No. 98, Seq. ID No. 100, Seq. ID No. 104, Seq. ID No. 106, Seq. ID No. 108, Seq. ID No. 110, Seq. ID No. 112, Seq. ID No. 114, Seq. ID No. 116, Seq. ID No. 118, Seq. ID No. 120, Seq. ID No. 122, Seq. ID No. 128, Seq. ID No. 130, Seq. ID No. 132, Seq. ID No. 134, Seq. ID No. 136, Seq. ID No. 138, Seq. ID No. 140, Seq. ID No. 144, Seq. ID No. 146, Seq. ID No. 148, Seq. ID No. 150, Seq. ID No. 154, Seq. ID No. 156, Seq. ID No. 158, Seq. ID No. 160, Seq. ID No. 162, Seq. ID No. 164, Seq. ID No. 166, Seq. ID No. 168, Seq. ID No. 170, Seq. ID No. 172, Seq. ID No. 176, Seq. ID No. 178, Seq. ID No. 180, Seq. ID No. 182, Seq. ID No. 184, Seq. ID No. 188, Seq. ID No. 192, Seq. ID No. 194, Seq. ID No. 196, Seq. ID No. 202, Seq. ID No. 206, Seq. ID No. 208, Seq. ID No. 216, Seq. ID No. 220, Seq. ID No. 222, Seq. ID No. 224, Seq. ID No. 226, Seq. ID No. 228, Seq. ID No. 232, Seq. ID No. 234, Seq. ID No. 240, Seq. ID No. 244, Seq. ID No. 246, Seq. ID No. 248, Seq. ID No. 254, Seq. ID No. 258, Seq. ID No. 260, Seq. ID No. 262, Seq. ID No. 264, Seq. ID No. 266, Seq. ID No. 268, Seq. ID No. 270, Seq. ID No. 274, Seq. ID No. 278, Seq. ID No. 280, Seq. ID No. 282, Seq. ID No. 284, Seq. ID No. 292, Seq. ID No. 294, Seq. ID No. 298, Seq. ID No. 302, Seq. ID No. 306, Seq. ID No. 308, Seq. ID No. 310, Seq. ID No. 314, Seq. ID No. 318, Seq. ID No. 322, Seq. ID No. 324, Seq. ID No. 326, Seq. ID No. 332, Seq. ID No. 334, Seq. ID No. 336, Seq. ID No. 338, Seq. ID No. 340, Seq. ID No. 344, Seq. ID No. 346, Seq. ID No. 348, Seq. ID No. 350, Seq. ID No. 354, Seq. ID No. 356, Seq. ID No. 358, Seq. ID No. 360, Seq. ID No. 362, Seq. ID No. 364, Seq. ID No. 366, Seq. ID No. 368, Seq. ID No. 370, Seq. ID No. 372, Seq. ID No. 376, Seq. ID No. 378, Seq. ID No. 380, Seq. ID No. 384, Seq. ID No. 386, Seq. ID No. 388, Seq. ID No. 390, Seq. ID No. 392, Seq. ID No. 394, Seq. ID No. 398, Seq. ID No. 402, Seq. ID No. 404, Seq. ID No. 406, Seq. ID No. 408, Seq. ID No. 410, Seq. ID No. 416, Seq. ID No. 424, Seq. ID No. 426, Seq. ID No. 428, Seq. ID No. 430, Seq. ID No. 432, Seq. ID No. 434, Seq. ID No. 436, Seq. ID No. 438, Seq. ID No. 440, Seq. ID No. 442, Seq. ID No. 444, Seq. ID No. 446, Seq. ID No. 448, Seq. ID No. 452, Seq. ID No. 454, Seq. ID No. 456, Seq. ID No. 458, Seq. ID No. 462, Seq. ID No. 464, Seq. ID No. 468, Seq. ID No. 470, Seq. ID No. 472, Seq. ID No. 474, Seq. ID No. 476, Seq. ID No. 478, Seq. ID No. 480, Seq. ID No. 482, Seq. ID No. 484, Seq. ID No. 486, Seq. ID No. 490, Seq. ID No. 492, Seq. ID No. 494, Seq. ID No. 496, Seq. ID No. 502, Seq. ID No. 506, Seq. ID No. 508, Seq. ID No. 512, Seq. ID No. 516, Seq. ID No. 518, Seq. ID No. 524, Seq. ID No. 530, Seq. ID No. 532, Seq. ID No. 534, Seq. ID No. 538, Seq. ID No. 540, Seq. ID No. 544, Seq. ID No. 548, Seq. ID No. 550, Seq. ID No. 552, Seq. ID No. 554, Seq. ID No. 556, Seq. ID No. 558, Seq. ID No. 560, Seq. ID No. 562, Seq. ID No. 566, Seq. ID No. 570, Seq. ID No. 572, Seq. ID No. 574, Seq. ID No. 576, Seq. ID No. 580, Seq. ID No. 582, Seq. ID No. 586, Seq. ID No. 588, Seq. ID No. 590, Seq. ID No. 592, Seq. ID No. 594, Seq. ID No. 596, Seq. ID No. 598, Seq. ID No. 600, Seq. ID No. 602, Seq. ID No. 604, Seq. ID No. 606, Seq. ID No. 610, Seq. ID No. 614, Seq. ID No. 616, Seq. ID No. 618, Seq. ID No. 620, Seq. ID No. 622, Seq. ID No. 624, Seq. ID No. 626, Seq. ID No. 628, Seq. ID No. 636, Seq. ID No. 638, Seq. ID No. 644, Seq. ID No. 646, Seq. ID No. 648, Seq. ID No. 650, Seq. ID No. 656, Seq. ID No. 664, Seq. ID No. 666, Seq. ID No. 668, Seq. ID No. 670, Seq. ID No. 676, Seq. ID No. 682, Seq. ID No. 684, Seq. ID No. 688, Seq. ID No. 690, Seq. ID No. 692, Seq. ID No. 696, Seq. ID No. 698, Seq. ID No. 700, Seq. ID No. 702, Seq. ID No. 704, Seq. ID No. 706, Seq. ID No. 708, Seq. ID No. 710, Seq. ID No. 714, Seq. ID No. 726, Seq. ID No. 728, Seq. ID No. 730, Seq. ID No. 732, Seq. ID No. 734, Seq. ID No. 736, Seq. ID No. 740, Seq. ID No. 742, Seq. ID No. 746, Seq. ID No. 748, Seq. ID No. 750, Seq. ID No. 752, Seq. ID No. 754, Seq. ID No. 760, Seq. ID No. 764, Seq. ID No. 766, Seq. ID No. 774, Seq. ID No. 776, Seq. ID No. 782, Seq. ID No. 784, Seq. ID No. 788, Seq. ID No. 794, Seq. ID No. 796, Seq. ID No. 800, Seq. ID No. 802, Seq. ID No. 806, Seq. ID No. 808, Seq. ID No. 810, Seq. ID No. 812, Seq. ID No. 814, Seq. ID No. 816, Seq. ID No. 818, Seq. ID No. 820, Seq. ID No. 822, Seq. ID No. 824, Seq. ID No. 828, Seq. ID No. 830, Seq. ID No. 832, Seq. ID No. 836, Seq. ID No. 838, Seq. ID No. 840, Seq. ID No. 844, Seq. ID No. 846, Seq. ID No. 848, Seq. ID No. 856, Seq. ID No. 860, Seq. ID No. 862, Seq. ID No. 866, Seq. ID No. 868, Seq. ID No. 872, Seq. ID No. 874, Seq. ID No. 876, Seq. ID No. 878, Seq. ID No. 880, Seq. ID No. 882, Seq. ID No. 886, Seq. ID No. 896, Seq. ID No. 898, Seq. ID No. 900, Seq. ID No. 902, Seq. ID No. 904, Seq. ID No. 906, Seq. ID No. 908, Seq. ID No. 910, Seq. ID No. 914, Seq. ID No. 916, Seq. ID No. 922, Seq. ID No. 924, Seq. ID No. 926, Seq. ID No. 932, Seq. ID No. 934, Seq. ID No. 936, Seq. ID No. 938, Seq. ID No. 940, Seq. ID No. 942, Seq. ID No. 944, Seq. ID No. 948, Seq. ID No. 950, Seq. ID No. 952, Seq. ID No. 954, Seq. ID No. 956, Seq. ID No. 958, Seq. ID No. 962, Seq. ID No. 964, Seq. ID No. 966, Seq. ID No. 968, Seq. ID No. 970, Seq. ID No. 972, Seq. ID No. 974, Seq. ID No. 976, Seq. ID No. 982, Seq. ID No. 984, Seq. ID No. 986, Seq. ID No. 988, Seq. ID No. 990, Seq. ID No. 992, Seq. ID No. 994, Seq. ID No. 996, Seq. ID No. 998, Seq. ID No. 1002, Seq. ID No. 1004, Seq. ID No. 1006, Seq. ID No. 1008, Seq. ID No. 1010, Seq. ID No. 1018, Seq. ID No. 1020, Seq. ID No. 1024, Seq. ID No. 1026, Seq. ID No. 1028, Seq. ID No. 1040, Seq. ID No. 1042, Seq. ID No. 1044, Seq. ID No. 1046, Seq. ID No. 1048, Seq. ID No. 1050, Seq. ID No. 1052, Seq. ID No. 1058, Seq. ID No. 1060, Seq. ID No. 1062, Seq. ID No. 1066, Seq. ID No. 1070, Seq. ID No. 1076, Seq. ID No. 1078, Seq. ID No. 1080, Seq. ID No. 1082, Seq. ID No. 1084, Seq. ID No. 1086, Seq. ID No. 1088, Seq. ID No. 1092, Seq. ID No. 1094, Seq. ID No. 1096, Seq. ID No. 1098, Seq. ID No. 1100, Seq. ID No. 1102, Seq. ID No. 1104, Seq. ID No. 1108, Seq. ID No. 1110, Seq. ID No. 1112, Seq. ID No. 1114, Seq. ID No. 1116, Seq. ID No. 1120, Seq. ID No. 1122, Seq. ID No. 1124, Seq. ID No. 1126, Seq. ID No. 1128, Seq. ID No. 1130, Seq. ID No. 1132, Seq. ID No. 1138, Seq. ID No. 1140, Seq. ID No. 1142, Seq. ID No. 1144, Seq. ID No. 1146, Seq. ID No. 1148, Seq. ID No. 1150, Seq. ID No. 1152, Seq. ID No. 1154, Seq. ID No. 1156, Seq. ID No. 1158, Seq. ID No. 1160, Seq. ID No. 1162, Seq. ID No. 1164, Seq. ID No. 1172, Seq. ID No. 1176, Seq. ID No. 1178, Seq. ID No. 1180, Seq. ID No. 1182, Seq. ID No. 1188, Seq. ID No. 1192, Seq. ID No. 1194, Seq. ID No. 1196, Seq. ID No. 1198, Seq. ID No. 1202, Seq. ID No. 1204, Seq. ID No. 1206, Seq. ID No. 1208, Seq. ID No. 1212, Seq. ID No. 1214, Seq. ID No. 1216, Seq. ID No. 1218, Seq. ID No. 1222, Seq. ID No. 1224, Seq. ID No. 1226, Seq. ID No. 1232, Seq. ID No. 1234, Seq. ID No. 1238, Seq. ID No. 1240, Seq. ID No. 1242, Seq. ID No. 1246, Seq. ID No. 1248, Seq. ID No. 1250, Seq. ID No. 1252, Seq. ID No. 1254, Seq. ID No. 1256, Seq. ID No. 1258, Seq. ID No. 1260, Seq. ID No. 1262, Seq. ID No. 1264, Seq. ID No. 1272, Seq. ID No. 1274, Seq. ID No. 1276, Seq. ID No. 1278, Seq. ID No. 1280, Seq. ID No. 1286, Seq. ID No. 1290, Seq. ID No. 1292, Seq. ID No. 1294, Seq. ID No. 1296, Seq. ID No. 1298, Seq. ID No. 1300, Seq. ID No. 1302, Seq. ID No. 1304, Seq. ID No. 1306, Seq. ID No. 1308, Seq. ID No. 1310, Seq. ID No. 1312, Seq. ID No. 1314, Seq. ID No. 1318, Seq. ID No. 1320, Seq. ID No. 1322, Seq. ID No. 1332, Seq. ID No. 1334, Seq. ID No. 1336, Seq. ID No. 1338, Seq. ID No. 1340, Seq. ID No. 1344, Seq. ID No. 1346, Seq. ID No. 1348, Seq. ID No. 1350, Seq. ID No. 1352, Seq. ID No. 1360, Seq. ID No. 1362, Seq. ID No. 1364, Seq. ID No. 1366, Seq. ID No. 1370, Seq. ID No. 1372, Seq. ID No. 1376, Seq. ID No. 1378, Seq. ID No. 1382, Seq. ID No. 1384, Seq. ID No. 1386, Seq. ID No. 1388, Seq. ID No. 1390, Seq. ID No. 1392, Seq. ID No. 1394, Seq. ID No. 1402, Seq. ID No. 1404, Seq. ID No. 1408, Seq. ID No. 1410, Seq. ID No. 1412, Seq. ID No. 1414, Seq. ID No. 1416, Seq. ID No. 1420, Seq. ID No. 1422, Seq. ID No. 1424, Seq. ID No. 1426, Seq. ID No. 1428, Seq. ID No. 1430, Seq. ID No. 1434, Seq. ID No. 1438, Seq. ID No. 1442, Seq. ID No. 1444, Seq. ID No. 1448, Seq. ID No. 1450, Seq. ID No. 1452, Seq. ID No. 1456, Seq. ID No. 1460, Seq. ID No. 1464, Seq. ID No. 1466, Seq. ID No. 1468, Seq. ID No. 1470, Seq. ID No. 1472, Seq. ID No. 1474, Seq. ID No. 1476, Seq. ID No. 1478, Seq. ID No. 1480, Seq. ID No. 1484, Seq. ID No. 1486, Seq. ID No. 1488, Seq. ID No. 1492, Seq. ID No. 1494, Seq. ID No. 1496, Seq. ID No. 1498, Seq. ID No. 1500, Seq. ID No. 1502, Seq. ID No. 1506, Seq. ID No. 1508, Seq. ID No. 1512, Seq. ID No. 1518, Seq. ID No. 1520, Seq. ID No. 1524, Seq. ID No. 1526, Seq. ID No. 1528, Seq. ID No. 1532, Seq. ID No. 1540, Seq. ID No. 1544, Seq. ID No. 1550, Seq. ID No. 1552, Seq. ID No. 1554, Seq. ID No. 1556, Seq. ID No. 1558, Seq. ID No. 1562, Seq. ID No. 1564, Seq. ID No. 1566, Seq. ID No. 1568, Seq. ID No. 1572, Seq. ID No. 1574, Seq. ID No. 1576, Seq. ID No. 1578, Seq. ID No. 1580, Seq. ID No. 1582, Seq. ID No. 1590, Seq. ID No. 1594, Seq. ID No. 1596, Seq. ID No. 1598, Seq. ID No. 1600, Seq. ID No. 1602, Seq. ID No. 1608, Seq. ID No. 1612, Seq. ID No. 1614, Seq. ID No. 1616, Seq. ID No. 1618, Seq. ID No. 1620, Seq. ID No. 1624, Seq. ID No. 1626, Seq. ID No. 1628, Seq. ID No. 1634, Seq. ID No. 1638, Seq. ID No. 1640, Seq. ID No. 1642, Seq. ID No. 1644, Seq. ID No. 1648, Seq. ID No. 1652, Seq. ID No. 1654, Seq. ID No. 1656, Seq. ID No. 1658, Seq. ID No. 1662, Seq. ID No. 1664, Seq. ID No. 1666, Seq. ID No. 1670, Seq. ID No. 1676, Seq. ID No. 1680, Seq. ID No. 1682, Seq. ID No. 1686, Seq. ID No. 1688, Seq. ID No. 1690, Seq. ID No. 1692, Seq. ID No. 1694, Seq. ID No. 1696, Seq. ID No. 1698, Seq. ID No. 1700, Seq. ID No. 1704, Seq. ID No. 1706, Seq. ID No. 1708, Seq. ID No. 1712, Seq. ID No. 1714, Seq. ID No. 1716, Seq. ID No. 1718, Seq. ID No. 1720, Seq. ID No. 1722, Seq. ID No. 1724, Seq. ID No. 1728, Seq. ID No. 1730, Seq. ID No. 1736, Seq. ID No. 1738, Seq. ID No. 1740, Seq. ID No. 1742, Seq. ID No. 1748, Seq. ID No. 1750, Seq. ID No. 1752, Seq. ID No. 1756, Seq. ID No. 1758, Seq. ID No. 1760, Seq. ID No. 1764, Seq. ID No. 1770, Seq. ID No. 1772, Seq. ID No. 1776, Seq. ID No. 1778, Seq. ID No. 1780, Seq. ID No. 1782, Seq. ID No. 1784, Seq. ID No. 1786, Seq. ID No. 1790, Seq. ID No. 1792, Seq. ID No. 1794, Seq. ID No. 1796, Seq. ID No. 1798, Seq. ID No. 1806, Seq. ID No. 1810, Seq. ID No. 1812, Seq. ID No. 1814, Seq. ID No. 1816, Seq. ID No. 1818, Seq. ID No. 1820, Seq. ID No. 1822, Seq. ID No. 1824, Seq. ID No. 1826, Seq. ID No. 1830, Seq. ID No. 1834, Seq. ID No. 1842, Seq. ID No. 1844, Seq. ID No. 1846, Seq. ID No. 1848, Seq. ID No. 1850, Seq. ID No. 1854, Seq. ID No. 1856, Seq. ID No. 1858, Seq. ID No. 1860, Seq. ID No. 1862, Seq. ID No. 1864, Seq. ID No. 1866, Seq. ID No. 1868, Seq. ID No. 1872, Seq. ID No. 1874, Seq. ID No. 1876, Seq. ID No. 1878, Seq. ID No. 1882, Seq. ID No. 1884, Seq. ID No. 1886, Seq. ID No. 1888, Seq. ID No. 1890, Seq. ID No. 1896, Seq. ID No. 1904, Seq. ID No. 1906, Seq. ID No. 1908, Seq. ID No. 1912, Seq. ID No. 1914, Seq. ID No. 1916, Seq. ID No. 1920, Seq. ID No. 1924, Seq. ID No. 1926, Seq. ID No. 1928, Seq. ID No. 1930, Seq. ID No. 1932, Seq. ID No. 1936, Seq. ID No. 1942, Seq. ID No. 1944, Seq. ID No. 1946, Seq. ID No. 1948, Seq. ID No. 1950, Seq. ID No. 1952, Seq. ID No. 1954, Seq. ID No. 1960, Seq. ID No. 1962, Seq. ID No. 1964, Seq. ID No. 1966, Seq. ID No. 1968, Seq. ID No. 1970, Seq. ID No. 1972, Seq. ID No. 1980, Seq. ID No. 1984, Seq. ID No. 1986, Seq. ID No. 1988, Seq. ID No. 1990, Seq. ID No. 1992, Seq. ID No. 1996, Seq. ID No. 1998, Seq. ID No. 2000, Seq. ID No. 2002, Seq. ID No. 2004, Seq. ID No. 2006, Seq. ID No. 2008, Seq. ID No. 2016, Seq. ID No. 2020, Seq. ID No. 2022, Seq. ID No. 2026, Seq. ID No. 2028, Seq. ID No. 2030, Seq. ID No. 2032, Seq. ID No. 2036, Seq. ID No. 2038, Seq. ID No. 2042, Seq. ID No. 2044, Seq. ID No. 2046, Seq. ID No. 2048, Seq. ID No. 2054, Seq. ID No. 2056, Seq. ID No. 2060, Seq. ID No. 2064, Seq. ID No. 2066, Seq. ID No. 2068, Seq. ID No. 2070, Seq. ID No. 2072, Seq. ID No. 2074, Seq. ID No. 2076, Seq. ID No. 2078, Seq. ID No. 2080, Seq. ID No. 2082, Seq. ID No. 2084, Seq. ID No. 2086, Seq. ID No. 2090, Seq. ID No. 2092, Seq. ID No. 2094, Seq. ID No. 2098, Seq. ID No. 2100, Seq. ID No. 2102, Seq. ID No. 2104, Seq. ID No. 2108, Seq. ID No. 2110, Seq. ID No. 2112, Seq. ID No. 2116, Seq. ID No. 2118, Seq. ID No. 2120, Seq. ID No. 2126, Seq. ID No. 2128, Seq. ID No. 2130, Seq. ID No. 2132, Seq. ID No. 2134, Seq. ID No. 2136, Seq. ID No. 2138, Seq. ID No. 2142, Seq. ID No. 2144, Seq. ID No. 2146, Seq. ID No. 2150, Seq. ID No. 2152, Seq. ID No. 2154, Seq. ID No. 2156, Seq. ID No. 2158, Seq. ID No. 2160, Seq. ID No. 2162, Seq. ID No. 2166, Seq. ID No. 2168, Seq. ID No. 2170, Seq. ID No. 2180, Seq. ID No. 2182, Seq. ID No. 2184, Seq. ID No. 2190, Seq. ID No. 2192, Seq. ID No. 2196, Seq. ID No. 2198, Seq. ID No. 2200, Seq. ID No. 2202, Seq. ID No. 2204, Seq. ID No. 2206, Seq. ID No. 2208, Seq. ID No. 2210, Seq. ID No. 2212, Seq. ID No. 2214, Seq. ID No. 2216, Seq. ID No. 2218, Seq. ID No. 2222, Seq. ID No. 2224, Seq. ID No. 2228, Seq. ID No. 2230, Seq. ID No. 2236, Seq. ID No. 2242, Seq. ID No. 2246, Seq. ID No. 2248, Seq. ID No. 2252, Seq. ID No. 2254, Seq. ID No. 2258, Seq. ID No. 2260, Seq. ID No. 2262, Seq. ID No. 2264, Seq. ID No. 2266, Seq. ID No. 2268, Seq. ID No. 2270, Seq. ID No. 2276, Seq. ID No. 2278, Seq. ID No. 2280, Seq. ID No. 2286, Seq. ID No. 2288, Seq. ID No. 2298, Seq. ID No. 2300, Seq. ID No. 2302, Seq. ID No. 2310, Seq. ID No. 2312, Seq. ID No. 2316, Seq. ID No. 2328, Seq. ID No. 2330, Seq. ID No. 2334, Seq. ID No. 2344, Seq. ID No. 2346, Seq. ID No. 2350, Seq. ID No. 2352, Seq. ID No. 2358, Seq. ID No. 2364, Seq. ID No. 2366, Seq. ID No. 2368, Seq. ID No. 2370, Seq. ID No. 2372, Seq. ID No. 2374, Seq. ID No. 2376, Seq. ID No. 2380, Seq. ID No. 2382, Seq. ID No. 2386, Seq. ID No. 2388, Seq. ID No. 2392, Seq. ID No. 2394, Seq. ID No. 2396, Seq. ID No. 2398, Seq. ID No. 2400, Seq. ID No. 2402, Seq. ID No. 2406, Seq. ID No. 2410, Seq. ID No. 2414, Seq. ID No. 2416, Seq. ID No. 2418, Seq. ID No. 2422, Seq. ID No. 2424, Seq. ID No. 2426, Seq. ID No. 2428, Seq. ID No. 2430, Seq. ID No. 2436, Seq. ID No. 2438, Seq. ID No. 2446, Seq. ID No. 2448, Seq. ID No. 2450, Seq. ID No. 2452, Seq. ID No. 2454, Seq. ID No. 2456, Seq. ID No. 2458, Seq. ID No. 2460, Seq. ID No. 2462, Seq. ID No. 2464, Seq. ID No. 2466, Seq. ID No. 2470, Seq. ID No. 2472, Seq. ID No. 2476, Seq. ID No. 2482, Seq. ID No. 2484, Seq. ID No. 2486, Seq. ID No. 2492, Seq. ID No. 2494, Seq. ID No. 2496, Seq. ID No. 2500, Seq. ID No. 2502, Seq. ID No. 2504, Seq. ID No. 2506, Seq. ID No. 2508, Seq. ID No. 2512, Seq. ID No. 2514, Seq. ID No. 2518, Seq. ID No. 2520, Seq. ID No. 2522, Seq. ID No. 2526, Seq. ID No. 2530, Seq. ID No. 2534, Seq. ID No. 2536, Seq. ID No. 2538, Seq. ID No. 2540, Seq. ID No. 2548, Seq. ID No. 2550, Seq. ID No. 2552, Seq. ID No. 2554, Seq. ID No. 2560, Seq. ID No. 2562, Seq. ID No. 2564, Seq. ID No. 2566, Seq. ID No. 2574, Seq. ID No. 2576, Seq. ID No. 2578, Seq. ID No. 2580, Seq. ID No. 2582, Seq. ID No. 2588, Seq. ID No. 2590, Seq. ID No. 2592, Seq. ID No. 2594, Seq. ID No. 2596, Seq. ID No. 2600, Seq. ID No. 2604, Seq. ID No. 2606, Seq. ID No. 2608, Seq. ID No. 2610, Seq. ID No. 2612, Seq. ID No. 2614, Seq. ID No. 2618, Seq. ID No. 2620, Seq. ID No. 2624, Seq. ID No. 2626, Seq. ID No. 2628, Seq. ID No. 2630, Seq. ID No. 2632, Seq. ID No. 2634, Seq. ID No. 2636, Seq. ID No. 2638, Seq. ID No. 2640, Seq. ID No. 2642, Seq. ID No. 2644, Seq. ID No. 2646, Seq. ID No. 2648, Seq. ID No. 2650, Seq. ID No. 2652, Seq. ID No. 2654, Seq. ID No. 2656, Seq. ID No. 2660, Seq. ID No. 2664, Seq. ID No. 2668, Seq. ID No. 2670, Seq. ID No. 2672, Seq. ID No. 2676, Seq. ID No. 2678, Seq. ID No. 2684, Seq. ID No. 2686, Seq. ID No. 2688, Seq. ID No. 2690, Seq. ID No. 2692, Seq. ID No. 2694, Seq. ID No. 2696, Seq. ID No. 2698, Seq. ID No. 2700, Seq. ID No. 2704, Seq. ID No. 2710, Seq. ID No. 2712, Seq. ID No. 2716, Seq. ID No. 2718, Seq. ID No. 2720, Seq. ID No. 2724, Seq. ID No. 2726, Seq. ID No. 2728, Seq. ID No. 2730, Seq. ID No. 2732, Seq. ID No. 2736, Seq. ID No. 2738, Seq. ID No. 2740, Seq. ID No. 2750, Seq. ID No. 2752, Seq. ID No. 2754, Seq. ID No. 2756, Seq. ID No. 2760, Seq. ID No. 2766, Seq. ID No. 2768, Seq. ID No. 2770, Seq. ID No. 2772, Seq. ID No. 2774, Seq. ID No. 2776, Seq. ID No. 2780, Seq. ID No. 2782, Seq. ID No. 2784, Seq. ID No. 2788, Seq. ID No. 2790, Seq. ID No. 2792, Seq. ID No. 2794, Seq. ID No. 2796, Seq. ID No. 2798, Seq. ID No. 2800, Seq. ID No. 2802, Seq. ID No. 2804, Seq. ID No. 2808, Seq. ID No. 2810, Seq. ID No. 2814, Seq. ID No. 2816, Seq. ID No. 2822, Seq. ID No. 2824, Seq. ID No. 2828, Seq. ID No. 2830, Seq. ID No. 2832, Seq. ID No. 2834, Seq. ID No. 2840, Seq. ID No. 2842, Seq. ID No. 2844, Seq. ID No. 2846, Seq. ID No. 2848, Seq. ID No. 2856, Seq. ID No. 2862, Seq. ID No. 2864, Seq. ID No. 2866, Seq. ID No. 2868, Seq. ID No. 2870, Seq. ID No. 2872, Seq. ID No. 2878, Seq. ID No. 2882, Seq. ID No. 2884, Seq. ID No. 2886, Seq. ID No. 2888, Seq. ID No. 2892, Seq. ID No. 2894, Seq. ID No. 2896, Seq. ID No. 2900, Seq. ID No. 2902, Seq. ID No. 2908, Seq. ID No. 2916, Seq. ID No. 2918, Seq. ID No. 2920, Seq. ID No. 2922, Seq. ID No. 2924, Seq. ID No. 2928, Seq. ID No. 2932, Seq. ID No. 2934, Seq. ID No. 2936, Seq. ID No. 2944, Seq. ID No. 2946, Seq. ID No. 2952, Seq. ID No. 2954, Seq. ID No. 2958, Seq. ID No. 2960, Seq. ID No. 2962, Seq. ID No. 2964, Seq. ID No. 2966, Seq. ID No. 2968, Seq. ID No. 2972, Seq. ID No. 2974, Seq. ID No. 2976, Seq. ID No. 2978, Seq. ID No. 2980, Seq. ID No. 2984, Seq. ID No. 2986, Seq. ID No. 2988, Seq. ID No. 2992, Seq. ID No. 2994, Seq. ID No. 3000, Seq. ID No. 3002, Seq. ID No. 3006, Seq. ID No. 3010, Seq. ID No. 3012, Seq. ID No. 3016, Seq. ID No. 3018, Seq. ID No. 3020, Seq. ID No. 3022, Seq. ID No. 3028, Seq. ID No. 3034, Seq. ID No. 3036, Seq. ID No. 3038, Seq. ID No. 3040, Seq. ID No. 3044, Seq. ID No. 3046, Seq. ID No. 3048, Seq. ID No. 3050, Seq. ID No. 3052, Seq. ID No. 3054, Seq. ID No. 3056, Seq. ID No. 3058, Seq. ID No. 3060, Seq. ID No. 3068, Seq. ID No. 3070, Seq. ID No. 3072, Seq. ID No. 3080, Seq. ID No. 3084, Seq. ID No. 3086, Seq. ID No. 3088, Seq. ID No. 3092, Seq. ID No. 3094, Seq. ID No. 3100, Seq. ID No. 3102, Seq. ID No. 3104, Seq. ID No. 3110, Seq. ID No. 3112, Seq. ID No. 3116, Seq. ID No. 3118, Seq. ID No. 3120, Seq. ID No. 3122, Seq. ID No. 3124, Seq. ID No. 3126, Seq. ID No. 3130, Seq. ID No. 3132, Seq. ID No. 3134, Seq. ID No. 3136, Seq. ID No. 3138, Seq. ID No. 3140, Seq. ID No. 3142, Seq. ID No. 3144, Seq. ID No. 3148, Seq. ID No. 3150, Seq. ID No. 3152, Seq. ID No. 3154, Seq. ID No. 3156, Seq. ID No. 3158, Seq. ID No. 3160, Seq. ID No. 3162, Seq. ID No. 3166, Seq. ID No. 3168, Seq. ID No. 3170, Seq. ID No. 3172, Seq. ID No. 3174, Seq. ID No. 3180, Seq. ID No. 3182, Seq. ID No. 3184, Seq. ID No. 3186, Seq. ID No. 3188, Seq. ID No. 3190, Seq. ID No. 3192, Seq. ID No. 3194, Seq. ID No. 3196, Seq. ID No. 3198, Seq. ID No. 3204, Seq. ID No. 3206, Seq. ID No. 3208, Seq. ID No. 3210, Seq. ID No. 3212, Seq. ID No. 3218, Seq. ID No. 3224, Seq. ID No. 3226, Seq. ID No. 3228, Seq. ID No. 3230, Seq. ID No. 3236, Seq. ID No. 3250, Seq. ID No. 3252, Seq. ID No. 3254, Seq. ID No. 3256, Seq. ID No. 3258, Seq. ID No. 3260, Seq. ID No. 3264, Seq. ID No. 3266, Seq. ID No. 3270, Seq. ID No. 3272, Seq. ID No. 3274, Seq. ID No. 3278, Seq. ID No. 3282, Seq. ID No. 3284, Seq. ID No. 3286, Seq. ID No. 3290, Seq. ID No. 3292, Seq. ID No. 3294, Seq. ID No. 3300, Seq. ID No. 3302, Seq. ID No. 3304, Seq. ID No. 3306, Seq. ID No. 3308, Seq. ID No. 3310, Seq. ID No. 3312, Seq. ID No. 3314, Seq. ID No. 3316, Seq. ID No. 3320, Seq. ID No. 3322, Seq. ID No. 3324, Seq. ID No. 3326, Seq. ID No. 3332, Seq. ID No. 3336, Seq. ID No. 3338, Seq. ID No. 3340, Seq. ID No. 3342, Seq. ID No. 3346, Seq. ID No. 3348, Seq. ID No. 3350, Seq. ID No. 3354, Seq. ID No. 3356, Seq. ID No. 3360, Seq. ID No. 3362, Seq. ID No. 3364, Seq. ID No. 3366, Seq. ID No. 3368, Seq. ID No. 3370, Seq. ID No. 3372, Seq. ID No. 3374, Seq. ID No. 3376, Seq. ID No. 3378, Seq. ID No. 3380, Seq. ID No. 3382, Seq. ID No. 3384, Seq. ID No. 3398, Seq. ID No. 3400, Seq. ID No. 3402, Seq. ID No. 3408, Seq. ID No. 3410, Seq. ID No. 3414, Seq. ID No. 3416, Seq. ID No. 3420, Seq. ID No. 3426, Seq. ID No. 3428, Seq. ID No. 3432, Seq. ID No. 3434, Seq. ID No. 3436, Seq. ID No. 3440, Seq. ID No. 3442, Seq. ID No. 3444, Seq. ID No. 3446, Seq. ID No. 3448, Seq. ID No. 3456, Seq. ID No. 3458, Seq. ID No. 3460, Seq. ID No. 3462, Seq. ID No. 3466, Seq. ID No. 3468, Seq. ID No. 3472, Seq. ID No. 3474, Seq. ID No. 3476, Seq. ID No. 3480, Seq. ID No. 3484, Seq. ID No. 3486, Seq. ID No. 3488, Seq. ID No. 3490, Seq. ID No. 3496, Seq. ID No. 3498, Seq. ID No. 3500, Seq. ID No. 3502, Seq. ID No. 3506, Seq. ID No. 3508, Seq. ID No. 3510, Seq. ID No. 3512, Seq. ID No. 3514, Seq. ID No. 3518, Seq. ID No. 3520, Seq. ID No. 3522, Seq. ID No. 3526, Seq. ID No. 3530, Seq. ID No. 3532, Seq. ID No. 3534, Seq. ID No. 3536, Seq. ID No. 3538, Seq. ID No. 3540, Seq. ID No. 3542, Seq. ID No. 3544, Seq. ID No. 3546, Seq. ID No. 3548, Seq. ID No. 3550, Seq. ID No. 3552, Seq. ID No. 3554, Seq. ID No. 3556, Seq. ID No. 3558, Seq. ID No. 3560, Seq. ID No. 3562, Seq. ID No. 3564, Seq. ID No. 3566, Seq. ID No. 3568, Seq. ID No. 3570, Seq. ID No. 3572, Seq. ID No. 3574, Seq. ID No. 3576, Seq. ID No. 3580, Seq. ID No. 3582, Seq. ID No. 3584, Seq. ID No. 3588, Seq. ID No. 3590, Seq. ID No. 3596, Seq. ID No. 3598, Seq. ID No. 3600, Seq. ID No. 3604, Seq. ID No. 3606, Seq. ID No. 3608, Seq. ID No. 3610, Seq. ID No. 3612, Seq. ID No. 3614, Seq. ID No. 3616, Seq. ID No. 3618, Seq. ID No. 3620, Seq. ID No. 3622, Seq. ID No. 3624, Seq. ID No. 3628, Seq. ID No. 3630, Seq. ID No. 3636, Seq. ID No. 3638, Seq. ID No. 3640, Seq. ID No. 3642, Seq. ID No. 3646, Seq. ID No. 3648, Seq. ID No. 3650, Seq. ID No. 3652, Seq. ID No. 3654, Seq. ID No. 3658, Seq. ID No. 3660, Seq. ID No. 3664, Seq. ID No. 3666, Seq. ID No. 3668, Seq. ID No. 3672, Seq. ID No. 3674, Seq. ID No. 3676, Seq. ID No. 3678, Seq. ID No. 3680, Seq. ID No. 3682, Seq. ID No. 3684, Seq. ID No. 3686, Seq. ID No. 3688, Seq. ID No. 3690, Seq. ID No. 3692, Seq. ID No. 3694, Seq. ID No. 3696, Seq. ID No. 3700, Seq. ID No. 3702, Seq. ID No. 3704, Seq. ID No. 3706, Seq. ID No. 3708, Seq. ID No. 3710, Seq. ID No. 3714, Seq. ID No. 3716, Seq. ID No. 3718, Seq. ID No. 3728, Seq. ID No. 3730, Seq. ID No. 3734, Seq. ID No. 3738, Seq. ID No. 3740, Seq. ID No. 3742, Seq. ID No. 3746, Seq. ID No. 3748, Seq. ID No. 3760, Seq. ID No. 3768, Seq. ID No. 3770, Seq. ID No. 3772, Seq. ID No. 3780, Seq. ID No. 3784, Seq. ID No. 3786, Seq. ID No. 3790, Seq. ID No. 3792, Seq. ID No. 3796, Seq. ID No. 3798, Seq. ID No. 3804, Seq. ID No. 3806, Seq. ID No. 3808, Seq. ID No. 3810, Seq. ID No. 3812, Seq. ID No. 3814, Seq. ID No. 3816, Seq. ID No. 3818, Seq. ID No. 3820, Seq. ID No. 3824, Seq. ID No. 3826, Seq. ID No. 3832, Seq. ID No. 3834, Seq. ID No. 3836, Seq. ID No. 3842, Seq. ID No. 3844, Seq. ID No. 3848, Seq. ID No. 3856, Seq. ID No. 3858, Seq. ID No. 3864, Seq. ID No. 3868, Seq. ID No. 3872, Seq. ID No. 3874, Seq. ID No. 3876, Seq. ID No. 3878, Seq. ID No. 3882, Seq. ID No. 3884, Seq. ID No. 3886, Seq. ID No. 3894, Seq. ID No. 3896, Seq. ID No. 3900, Seq. ID No. 3902, Seq. ID No. 3906, Seq. ID No. 3908, Seq. ID No. 3912, Seq. ID No. 3914, Seq. ID No. 3916, Seq. ID No. 3920, Seq. ID No. 3922, Seq. ID No. 3924, Seq. ID No. 3926, Seq. ID No. 3928, Seq. ID No. 3930, Seq. ID No. 3932, Seq. ID No. 3934, Seq. ID No. 3936, Seq. ID No. 3940, Seq. ID No. 3942, Seq. ID No. 3944, Seq. ID No. 3946, Seq. ID No. 3948, Seq. ID No. 3950, Seq. ID No. 3952, Seq. ID No. 3954, Seq. ID No. 3956, Seq. ID No. 3958, Seq. ID No. 3960, Seq. ID No. 3962, Seq. ID No. 3966, Seq. ID No. 3968, Seq. ID No. 3970, Seq. ID No. 3972, Seq. ID No. 3976, Seq. ID No. 3980, Seq. ID No. 3982, Seq. ID No. 3984, Seq. ID No. 3986, Seq. ID No. 3988, Seq. ID No. 3992, Seq. ID No. 3996, Seq. ID No. 3998, Seq. ID No. 4000, Seq. ID No. 4004, Seq. ID No. 4008, Seq. ID No. 4010, Seq. ID No. 4016, Seq. ID No. 4018, Seq. ID No. 4022, Seq. ID No. 4024, Seq. ID No. 4030, Seq. ID No. 4036, Seq. ID No. 4038, Seq. ID No. 4040, Seq. ID No. 4050, Seq. ID No. 4052, Seq. ID No. 4054, Seq. ID No. 4056, Seq. ID No. 4058, Seq. ID No. 4062, Seq. ID No. 4076, Seq. ID No. 4080, Seq. ID No. 4086, Seq. ID No. 4088, Seq. ID No. 4090, Seq. ID No. 4092, Seq. ID No. 4094, Seq. ID No. 4096, Seq. ID No. 4100, Seq. ID No. 4106, Seq. ID No. 4108, Seq. ID No. 4110, Seq. ID No. 4112, Seq. ID No. 4114, Seq. ID No. 4118, Seq. ID No. 4120, Seq. ID No. 4122, Seq. ID No. 4124, Seq. ID No. 4126, Seq. ID No. 4128, Seq. ID No. 4130, Seq. ID No. 4132, Seq. ID No. 4134, Seq. ID No. 4136, Seq. ID No. 4138, Seq. ID No. 4140, Seq. ID No. 4142, Seq. ID No. 4144, Seq. ID No. 4146, Seq. ID No. 4150, Seq. ID No. 4154, Seq. ID No. 4156, Seq. ID No. 4158, Seq. ID No. 4162, Seq. ID No. 4164, Seq. ID No. 4168, Seq. ID No. 4172, Seq. ID No. 4174, Seq. ID No. 4176, Seq. ID No. 4178, Seq. ID No. 4180, Seq. ID No. 4182, Seq. ID No. 4186, Seq. ID No. 4188, Seq. ID No. 4190, Seq. ID No. 4192, Seq. ID No. 4194, Seq. ID No. 4196, Seq. ID No. 4198, Seq. ID No. 4200, Seq. ID No. 4204, Seq. ID No. 4206, Seq. ID No. 4208, Seq. ID No. 4210, Seq. ID No. 4212, Seq. ID No. 4220, Seq. ID No. 4230, Seq. ID No. 4234, Seq. ID No. 4236, Seq. ID No. 4238, Seq. ID No. 4240, Seq. ID No. 4244, Seq. ID No. 4250, Seq. ID No. 4252, Seq. ID No. 4254, Seq. ID No. 4256, Seq. ID No. 4260, Seq. ID No. 4264, Seq. ID No. 4266, Seq. ID No. 4268, Seq. ID No. 4270, Seq. ID No. 4272, Seq. ID No. 4274, Seq. ID No. 4276, Seq. ID No. 4282, Seq. ID No. 4284, Seq. ID No. 4286, Seq. ID No. 4288, Seq. ID No. 4290, Seq. ID No. 4292, Seq. ID No. 4294, Seq. ID No. 4296, Seq. ID No. 4298, Seq. ID No. 4300, Seq. ID No. 4302, Seq. ID No. 4304, Seq. ID No. 4308, Seq. ID No. 4310, Seq. ID No. 4314, Seq. ID No. 4316, Seq. ID No. 4318, Seq. ID No. 4320, Seq. ID No. 4322, Seq. ID No. 4324, Seq. ID No. 4326, Seq. ID No. 4330, Seq. ID No. 4332, Seq. ID No. 4334, Seq. ID No. 4336, Seq. ID No. 4338, Seq. ID No. 4340, Seq. ID No. 4344, Seq. ID No. 4346, Seq. ID No. 4348, Seq. ID No. 4352, Seq. ID No. 4354, Seq. ID No. 4356, Seq. ID No. 4360, Seq. ID No. 4362, Seq. ID No. 4364, Seq. ID No. 4366, Seq. ID No. 4368, Seq. ID No. 4370, Seq. ID No. 4376, Seq. ID No. 4378, Seq. ID No. 4380, Seq. ID No. 4384, Seq. ID No. 4390, Seq. ID No. 4392, Seq. ID No. 4394, Seq. ID No. 4396, Seq. ID No. 4398, Seq. ID No. 4400, Seq. ID No. 4402, Seq. ID No. 4404, Seq. ID No. 4406, Seq. ID No. 4408, Seq. ID No. 4410, Seq. ID No. 4412, Seq. ID No. 4414, Seq. ID No. 4416, Seq. ID No. 4418, Seq. ID No. 4420, Seq. ID No. 4422, Seq. ID No. 4424, Seq. ID No. 4426, Seq. ID No. 4428, Seq. ID No. 4430, Seq. ID No. 4432, Seq. ID No. 4436, Seq. ID No. 4438, Seq. ID No. 4440, Seq. ID No. 4442, Seq. ID No. 4450, Seq. ID No. 4452, Seq. ID No. 4454, Seq. ID No. 4456, Seq. ID No. 4458, Seq. ID No. 4460, Seq. ID No. 4462, Seq. ID No. 4466, Seq. ID No. 4468, Seq. ID No. 4472, Seq. ID No. 4476, Seq. ID No. 4478, Seq. ID No. 4480, Seq. ID No. 4482, Seq. ID No. 4486, Seq. ID No. 4488, Seq. ID No. 4490, Seq. ID No. 4492, Seq. ID No. 4494, Seq. ID No. 4496, Seq. ID No. 4498, Seq. ID No. 4502, Seq. ID No. 4504, Seq. ID No. 4506, Seq. ID No. 4508, Seq. ID No. 4510, Seq. ID No. 4512, Seq. ID No. 4514, Seq. ID No. 4516, Seq. ID No. 4520, Seq. ID No. 4522, Seq. ID No. 4524, Seq. ID No. 4526, Seq. ID No. 4528, Seq. ID No. 4530, Seq. ID No. 4532, Seq. ID No. 4534, Seq. ID No. 4538, Seq. ID No. 4542, Seq. ID No. 4546, Seq. ID No. 4548, Seq. ID No. 4550, Seq. ID No. 4552, Seq. ID No. 4554, Seq. ID No. 4556, Seq. ID No. 4560, Seq. ID No. 4562, Seq. ID No. 4566, Seq. ID No. 4568, Seq. ID No. 4572, Seq. ID No. 4574, Seq. ID No. 4576, Seq. ID No. 4578, Seq. ID No. 4582, Seq. ID No. 4586, Seq. ID No. 4590, Seq. ID No. 4592, Seq. ID No. 4598, Seq. ID No. 4600, Seq. ID No. 4602, Seq. ID No. 4604, Seq. ID No. 4606, Seq. ID No. 4608, Seq. ID No. 4610, Seq. ID No. 4612, Seq. ID No. 4614, Seq. ID No. 4616, Seq. ID No. 4618, Seq. ID No. 4620, Seq. ID No. 4622, Seq. ID No. 4624, Seq. ID No. 4628, Seq. ID No. 4632, Seq. ID No. 4638, Seq. ID No. 4642, Seq. ID No. 4644, Seq. ID No. 4646, Seq. ID No. 4648, Seq. ID No. 4650, Seq. ID No. 4654, Seq. ID No. 4660, Seq. ID No. 4662, Seq. ID No. 4664, Seq. ID No. 4666, Seq. ID No. 4668, Seq. ID No. 4670, Seq. ID No. 4672, Seq. ID No. 4674, Seq. ID No. 4676, Seq. ID No. 4678, Seq. ID No. 4680, Seq. ID No. 4684, Seq. ID No. 4686, Seq. ID No. 4688, Seq. ID No. 4692, Seq. ID No. 4694, Seq. ID No. 4696, Seq. ID No. 4700, Seq. ID No. 4702, Seq. ID No. 4704, Seq. ID No. 4706, Seq. ID No. 4714, Seq. ID No. 4718, Seq. ID No. 4720, Seq. ID No. 4722, Seq. ID No. 4728, Seq. ID No. 4730, Seq. ID No. 4734, Seq. ID No. 4736, Seq. ID No. 4738, Seq. ID No. 4740, Seq. ID No. 4742, Seq. ID No. 4744, Seq. ID No. 4748, Seq. ID No. 4750, Seq. ID No. 4752, Seq. ID No. 4756, Seq. ID No. 4758, Seq. ID No. 4764, Seq. ID No. 4766, Seq. ID No. 4768, Seq. ID No. 4770, Seq. ID No. 4774, Seq. ID No. 4776, Seq. ID No. 4778, Seq. ID No. 4780, Seq. ID No. 4782, Seq. ID No. 4788, Seq. ID No. 4790, Seq. ID No. 4792, Seq. ID No. 4794, Seq. ID No. 4796, Seq. ID No. 4798, Seq. ID No. 4802, Seq. ID No. 4804, Seq. ID No. 4806, Seq. ID No. 4810, Seq. ID No. 4812, Seq. ID No. 4814, Seq. ID No. 4816, Seq. ID No. 4818, Seq. ID No. 4820, Seq. ID No. 4826, Seq. ID No. 4828, Seq. ID No. 4830, Seq. ID No. 4832, Seq. ID No. 4834, Seq. ID No. 4838, Seq. ID No. 4840, Seq. ID No. 4842, Seq. ID No. 4844, Seq. ID No. 4846, Seq. ID No. 4850, Seq. ID No. 4852, Seq. ID No. 4854, Seq. ID No. 4856, Seq. ID No. 4862, Seq. ID No. 4864, Seq. ID No. 4868, Seq. ID No. 4876, Seq. ID No. 4878, Seq. ID No. 4880, Seq. ID No. 4882, Seq. ID No. 4884, Seq. ID No. 4890, Seq. ID No. 4892, Seq. ID No. 4894, Seq. ID No. 4896, Seq. ID No. 4898, Seq. ID No. 4900, Seq. ID No. 4902, Seq. ID No. 4904, Seq. ID No. 4906, Seq. ID No. 4908, Seq. ID No. 4910, Seq. ID No. 4912, Seq. ID No. 4914, Seq. ID No. 4916, Seq. ID No. 4918, Seq. ID No. 4920, Seq. ID No. 4922, Seq. ID No. 4924, Seq. ID No. 4928, Seq. ID No. 4932, Seq. ID No. 4936, Seq. ID No. 4938, Seq. ID No. 4940, Seq. ID No. 4942, Seq. ID No. 4944, Seq. ID No. 4946, Seq. ID No. 4948, Seq. ID No. 4950, Seq. ID No. 4952, Seq. ID No. 4954, Seq. ID No. 4958, Seq. ID No. 4960, Seq. ID No. 4962, Seq. ID No. 4964, Seq. ID No. 4974, Seq. ID No. 4976, Seq. ID No. 4978, Seq. ID No. 4980, Seq. ID No. 4982, Seq. ID No. 4984, Seq. ID No. 4986, Seq. ID No. 4988, Seq. ID No. 4990, Seq. ID No. 4992, Seq. ID No. 4994, Seq. ID No. 4996, Seq. ID No. 4998, Seq. ID No. 5000, Seq. ID No. 5004, Seq. ID No. 5006, Seq. ID No. 5008, Seq. ID No. 5010, Seq. ID No. 5012, Seq. ID No. 5014, Seq. ID No. 5018, Seq. ID No. 5020, Seq. ID No. 5022, Seq. ID No. 5024, Seq. ID No. 5026, Seq. ID No. 5030, Seq. ID No. 5032, Seq. ID No. 5034, Seq. ID No. 5036, Seq. ID No. 5042, Seq. ID No. 5044, Seq. ID No. 5046, Seq. ID No. 5048, Seq. ID No. 5058, Seq. ID No. 5060, Seq. ID No. 5064, Seq. ID No. 5066, Seq. ID No. 5068, Seq. ID No. 5070, Seq. ID No. 5076, Seq. ID No. 5078, Seq. ID No. 5080, Seq. ID No. 5082, Seq. ID No. 5084, Seq. ID No. 5086, Seq. ID No. 5088, Seq. ID No. 5092, Seq. ID No. 5100, Seq. ID No. 5102, Seq. ID No. 5104, Seq. ID No. 5110, Seq. ID No. 5112, Seq. ID No. 5114, Seq. ID No. 5116, Seq. ID No. 5118, Seq. ID No. 5122, Seq. ID No. 5126, Seq. ID No. 5128, Seq. ID No. 5134, Seq. ID No. 5140, Seq. ID No. 5142, Seq. ID No. 5146, Seq. ID No. 5148, Seq. ID No. 5150, Seq. ID No. 5152, Seq. ID No. 5156, Seq. ID No. 5158, Seq. ID No. 5160, Seq. ID No. 5162, Seq. ID No. 5166, Seq. ID No. 5168, Seq. ID No. 5170, Seq. ID No. 5178, Seq. ID No. 5182, Seq. ID No. 5184, Seq. ID No. 5186, Seq. ID No. 5188, Seq. ID No. 5190, Seq. ID No. 5192, Seq. ID No. 5194, Seq. ID No. 5196, Seq. ID No. 5198, Seq. ID No. 5200, Seq. ID No. 5202, Seq. ID No. 5204, Seq. ID No. 5206, Seq. ID No. 5210, Seq. ID No. 5212, Seq. ID No. 5216, Seq. ID No. 5220, Seq. ID No. 5222, Seq. ID No. 5230, Seq. ID No. 5232, Seq. ID No. 5240, Seq. ID No. 5242, Seq. ID No. 5244, Seq. ID No. 5246, Seq. ID No. 5252, Seq. ID No. 5254, Seq. ID No. 5256, Seq. ID No. 5262, Seq. ID No. 5264, Seq. ID No. 5266, Seq. ID No. 5268, Seq. ID No. 5278, Seq. ID No. 5280, Seq. ID No. 5282, Seq. ID No. 5286, Seq. ID No. 5288, Seq. ID No. 5292, Seq. ID No. 5296, Seq. ID No. 5298, Seq. ID No. 5300, Seq. ID No. 5302, Seq. ID No. 5304, Seq. ID No. 5306, Seq. ID No. 5308, Seq. ID No. 5310, Seq. ID No. 5312, Seq. ID No. 5314, Seq. ID No. 5318, Seq. ID No. 5320, Seq. ID No. 5322, Seq. ID No. 5324, Seq. ID No. 5328, Seq. ID No. 5330, Seq. ID No. 5332, Seq. ID No. 5334, Seq. ID No. 5336, Seq. ID No. 5338, Seq. ID No. 5344, Seq. ID No. 5346, Seq. ID No. 5350, Seq. ID No. 5352, Seq. ID No. 5358, Seq. ID No. 5360, Seq. ID No. 5366, Seq. ID No. 5368, Seq. ID No. 5374, Seq. ID No. 5378, Seq. ID No. 5380, Seq. ID No. 5382, Seq. ID No. 5384, Seq. ID No. 5388, Seq. ID No. 5390, Seq. ID No. 5392, Seq. ID No. 5394, Seq. ID No. 5398, Seq. ID No. 5400, Seq. ID No. 5402, Seq. ID No. 5404, Seq. ID No. 5406, Seq. ID No. 5408, Seq. ID No. 5410, Seq. ID No. 5412, Seq. ID No. 5416, Seq. ID No. 5418, Seq. ID No. 5420, Seq. ID No. 5422, Seq. ID No. 5424, Seq. ID No. 5426, Seq. ID No. 5428, Seq. ID No. 5432, Seq. ID No. 5434, Seq. ID No. 5436, Seq. ID No. 5438, Seq. ID No. 5440, Seq. ID No. 5442, Seq. ID No. 5444, Seq. ID No. 5448, Seq. ID No. 5450, Seq. ID No. 5452, Seq. ID No. 5454, Seq. ID No. 5456, Seq. ID No. 5458, Seq. ID No. 5468, Seq. ID No. 5472, Seq. ID No. 5474, Seq. ID No. 5478, Seq. ID No. 5480, Seq. ID No. 5482, Seq. ID No. 5492, Seq. ID No. 5494, Seq. ID No. 5496, Seq. ID No. 5500, Seq. ID No. 5504, Seq. ID No. 5510, Seq. ID No. 5514, Seq. ID No. 5516, Seq. ID No. 5518, Seq. ID No. 5520, Seq. ID No. 5522, Seq. ID No. 5528, Seq. ID No. 5530, Seq. ID No. 5532, Seq. ID No. 5534, Seq. ID No. 5538, Seq. ID No. 5540, Seq. ID No. 5542, Seq. ID No. 5544, Seq. ID No. 5546, Seq. ID No. 5548, Seq. ID No. 5550, Seq. ID No. 5552, Seq. ID No. 5554, Seq. ID No. 5558, Seq. ID No. 5560, Seq. ID No. 5564, Seq. ID No. 5568, Seq. ID No. 5570, Seq. ID No. 5576, Seq. ID No. 5578, Seq. ID No. 5582, Seq. ID No. 5584, Seq. ID No. 5586, Seq. ID No. 5590, Seq. ID No. 5592, Seq. ID No. 5594, Seq. ID No. 5596, Seq. ID No. 5600, Seq. ID No. 5602, Seq. ID No. 5608, Seq. ID No. 5614, Seq. ID No. 5616, Seq. ID No. 5618, Seq. ID No. 5620, Seq. ID No. 5622, Seq. ID No. 5626, Seq. ID No. 5628, Seq. ID No. 5630, Seq. ID No. 5632, Seq. ID No. 5638, Seq. ID No. 5640, Seq. ID No. 5642, Seq. ID No. 5646, Seq. ID No. 5648, Seq. ID No. 5650, Seq. ID No. 5652, Seq. ID No. 5656, Seq. ID No. 5658, Seq. ID No. 5662, Seq. ID No. 5666, Seq. ID No. 5668, Seq. ID No. 5670, Seq. ID No. 5678, Seq. ID No. 5680, Seq. ID No. 5684, Seq. ID No. 5686, Seq. ID No. 5688, Seq. ID No. 5690, Seq. ID No. 5692, Seq. ID No. 5694, Seq. ID No. 5698, Seq. ID No. 5700, Seq. ID No. 5702, Seq. ID No. 5704, Seq. ID No. 5706, Seq. ID No. 5708, Seq. ID No. 5712, Seq. ID No. 5714, Seq. ID No. 5716, Seq. ID No. 5718, Seq. ID No. 5720, Seq. ID No. 5722, Seq. ID No. 5724, Seq. ID No. 5726, Seq. ID No. 5728, Seq. ID No. 5736, Seq. ID No. 5740, Seq. ID No. 5742, Seq. ID No. 5744, Seq. ID No. 5746, Seq. ID No. 5748, Seq. ID No. 5750, Seq. ID No. 5752, Seq. ID No. 5754, Seq. ID No. 5756, Seq. ID No. 5758, Seq. ID No. 5760, Seq. ID No. 5762, Seq. ID No. 5770, Seq. ID No. 5772, Seq. ID No. 5778, Seq. ID No. 5780, Seq. ID No. 5784, Seq. ID No. 5788, Seq. ID No. 5792, Seq. ID No. 5796, Seq. ID No. 5800, Seq. ID No. 5802, Seq. ID No. 5808, Seq. ID No. 5810, Seq. ID No. 5814, Seq. ID No. 5816, Seq. ID No. 5820, Seq. ID No. 5822, Seq. ID No. 5824, Seq. ID No. 5826, Seq. ID No. 5828, Seq. ID No. 5830, Seq. ID No. 5832, Seq. ID No. 5834, Seq. ID No. 5836, Seq. ID No. 5838, Seq. ID No. 5840, Seq. ID No. 5842, Seq. ID No. 5846, Seq. ID No. 5848, Seq. ID No. 5850, Seq. ID No. 5852, Seq. ID No. 5856, Seq. ID No. 5858, Seq. ID No. 5860, Seq. ID No. 5862, Seq. ID No. 5864, Seq. ID No. 5866, Seq. ID No. 5870, Seq. ID No. 5872, Seq. ID No. 5874, Seq. ID No. 5876, Seq. ID No. 5880, Seq. ID No. 5882, Seq. ID No. 5884, Seq. ID No. 5890, Seq. ID No. 5896, Seq. ID No. 5898, Seq. ID No. 5902, Seq. ID No. 5904, Seq. ID No. 5912, Seq. ID No. 5914, Seq. ID No. 5916, Seq. ID No. 5918, Seq. ID No. 5920, Seq. ID No. 5928, Seq. ID No. 5930, Seq. ID No. 5934, Seq. ID No. 5936, Seq. ID No. 5938, Seq. ID No. 5942, Seq. ID No. 5946, Seq. ID No. 5948, Seq. ID No. 5952, Seq. ID No. 5960, Seq. ID No. 5962, Seq. ID No. 5964, Seq. ID No. 5966, Seq. ID No. 5968, Seq. ID No. 5970, Seq. ID No. 5972, Seq. ID No. 5974, Seq. ID No. 5978, Seq. ID No. 5980, Seq. ID No. 5982, Seq. ID No. 5986, Seq. ID No. 5988, Seq. ID No. 5990, Seq. ID No. 5992, Seq. ID No. 5994, Seq. ID No. 5996, Seq. ID No. 6000, Seq. ID No. 6002, Seq. ID No. 6004, Seq. ID No. 6010, Seq. ID No. 6012, Seq. ID No. 6014, Seq. ID No. 6016, Seq. ID No. 6018, Seq. ID No. 6026, Seq. ID No. 6028, Seq. ID No. 6030, Seq. ID No. 6032, Seq. ID No. 6034, Seq. ID No. 6036, Seq. ID No. 6040, Seq. ID No. 6042, Seq. ID No. 6044, Seq. ID No. 6046, Seq. ID No. 6050, Seq. ID No. 6052, Seq. ID No. 6054, Seq. ID No. 6056, Seq. ID No. 6058, Seq. ID No. 6062, Seq. ID No. 6064, Seq. ID No. 6066, Seq. ID No. 6070, Seq. ID No. 6072, Seq. ID No. 6074, Seq. ID No. 6078, Seq. ID No. 6080, Seq. ID No. 6086, Seq. ID No. 6088, Seq. ID No. 6090, Seq. ID No. 6094, Seq. ID No. 6096, Seq. ID No. 6102, Seq. ID No. 6104, Seq. ID No. 6110, Seq. ID No. 6112, Seq. ID No. 6118, Seq. ID No. 6122, Seq. ID No. 6124, Seq. ID No. 6130, Seq. ID No. 6132, Seq. ID No. 6134, Seq. ID No. 6136, Seq. ID No. 6138, Seq. ID No. 6140, Seq. ID No. 6142, Seq. ID No. 6146, Seq. ID No. 6152, Seq. ID No. 6156, Seq. ID No. 6158, Seq. ID No. 6160, Seq. ID No. 6166, Seq. ID No. 6168, Seq. ID No. 6170, Seq. ID No. 6172, Seq. ID No. 6176, Seq. ID No. 6178, Seq. ID No. 6182, Seq. ID No. 6188, Seq. ID No. 6194, Seq. ID No. 6196, Seq. ID No. 6198, Seq. ID No. 6200, Seq. ID No. 6202, Seq. ID No. 6210, Seq. ID No. 6214, Seq. ID No. 6216, Seq. ID No. 6218, Seq. ID No. 6220, Seq. ID No. 6232, Seq. ID No. 6234, Seq. ID No. 6236, Seq. ID No. 6240, Seq. ID No. 6242, Seq. ID No. 6244, Seq. ID No. 6250, Seq. ID No. 6258, Seq. ID No. 6260, Seq. ID No. 6262, Seq. ID No. 6264, Seq. ID No. 6268, Seq. ID No. 6270, Seq. ID No. 6272, Seq. ID No. 6276, Seq. ID No. 6278, Seq. ID No. 6280, Seq. ID No. 6282, Seq. ID No. 6284, Seq. ID No. 6286, Seq. ID No. 6288, Seq. ID No. 6294, Seq. ID No. 6296, Seq. ID No. 6298, Seq. ID No. 6300, Seq. ID No. 6306, Seq. ID No. 6310, Seq. ID No. 6312, Seq. ID No. 6316, Seq. ID No. 6318, Seq. ID No. 6320, Seq. ID No. 6322, Seq. ID No. 6324, Seq. ID No. 6326, Seq. ID No. 6328, Seq. ID No. 6330, Seq. ID No. 6332, Seq. ID No. 6336, Seq. ID No. 6338, Seq. ID No. 6342, Seq. ID No. 6346, Seq. ID No. 6350, Seq. ID No. 6352, Seq. ID No. 6354, Seq. ID No. 6360, Seq. ID No. 6362, Seq. ID No. 6368, Seq. ID No. 6370, Seq. ID No. 6372, Seq. ID No. 6374, Seq. ID No. 6376, Seq. ID No. 6378, Seq. ID No. 6380, Seq. ID No. 6382, Seq. ID No. 6386, Seq. ID No. 6390, Seq. ID No. 6394, Seq. ID No. 6396, Seq. ID No. 6400, Seq. ID No. 6410, Seq. ID No. 6412, Seq. ID No. 6414, Seq. ID No. 6418, Seq. ID No. 6422, Seq. ID No. 6424, Seq. ID No. 6426, Seq. ID No. 6428, Seq. ID No. 6430, Seq. ID No. 6432, Seq. ID No. 6442, Seq. ID No. 6444, Seq. ID No. 6446, Seq. ID No. 6452, Seq. ID No. 6454, Seq. ID No. 6460, Seq. ID No. 6462, Seq. ID No. 6464, Seq. ID No. 6466, Seq. ID No. 6468, Seq. ID No. 6470, Seq. ID No. 6472, Seq. ID No. 6474, Seq. ID No. 6480, Seq. ID No. 6484, Seq. ID No. 6494, Seq. ID No. 6496, Seq. ID No. 6498, Seq. ID No. 6502, Seq. ID No. 6504, Seq. ID No. 6506, Seq. ID No. 6508, Seq. ID No. 6512, Seq. ID No. 6518, Seq. ID No. 6524, Seq. ID No. 6526, Seq. ID No. 6536, Seq. ID No. 6538, Seq. ID No. 6540, Seq. ID No. 6542, Seq. ID No. 6544, Seq. ID No. 6546, Seq. ID No. 6548, Seq. ID No. 6550, Seq. ID No. 6552, Seq. ID No. 6554, Seq. ID No. 6556, Seq. ID No. 6558, Seq. ID No. 6560, Seq. ID No. 6564, Seq. ID No. 6574, Seq. ID No. 6576, Seq. ID No. 6578, Seq. ID No. 6580, Seq. ID No. 6584, Seq. ID No. 6586, Seq. ID No. 6588, Seq. ID No. 6592, Seq. ID No. 6598, Seq. ID No. 6602, Seq. ID No. 6604, Seq. ID No. 6606, Seq. ID No. 6610, Seq. ID No. 6612, Seq. ID No. 6614, Seq. ID No. 6616, Seq. ID No. 6618, Seq. ID No. 6620, Seq. ID No. 6622, Seq. ID No. 6624, Seq. ID No. 6628, Seq. ID No. 6630, Seq. ID No. 6632, Seq. ID No. 6634, Seq. ID No. 6638, Seq. ID No. 6640, Seq. ID No. 6644, Seq. ID No. 6646, and Seq. ID No. 6650.

In yet another preferred embodiment, the polypeptide is selected from the Alloiococcus otitidis polypeptides identified by the Applicants' assignee's ORF finder program that searches for a transmembrane domain between two Stop codons and a Start codon immediately upstream of this transmembrane region, and is selected from one of Seq. ID No. 8, Seq. ID No. 10, Seq. ID No. 34, Seq. ID No. 48, Seq. ID No. 68, Seq. ID No. 86, Seq. ID No. 88, Seq. ID No. 124, Seq. ID No. 134, Seq. ID No. 142, Seq. ID No. 160, Seq. ID No. 164, Seq. ID No. 192, Seq. ID No. 210, Seq. ID No. 212, Seq. ID No. 216, Seq. ID No. 218, Seq. ID No. 226, Seq. ID No. 236, Seq. ID No. 238, Seq. ID No. 250, Seq. ID No. 252, Seq. ID No. 256, Seq. ID No. 272, Seq. ID No. 278, Seq. ID No. 286, Seq. ID No. 288, Seq. ID No. 290, Seq. ID No. 328, Seq. ID No. 330, Seq. ID No. 374, Seq. ID No. 376, Seq. ID No. 420, Seq. ID No. 488, Seq. ID No. 498, Seq. ID No. 500, Seq. ID No. 504, Seq. ID No. 506, Seq. ID No. 510, Seq. ID No. 512, Seq. ID No. 516, Seq. ID No. 526, Seq. ID No. 528, Seq. ID No. 546, Seq. ID No. 568, Seq. ID No. 570, Seq. ID No. 576, Seq. ID No. 578, Seq. ID No. 580, Seq. ID No. 594, Seq. ID No. 618, Seq. ID No. 638, Seq. ID No. 640, Seq. ID No. 642, Seq. ID No. 646, Seq. ID No. 654, Seq. ID No. 656, Seq. ID No. 660, Seq. ID No. 670, Seq. ID No. 676, Seq. ID No. 678, Seq. ID No. 680, Seq. ID No. 716, Seq. ID No. 718, Seq. ID No. 720, Seq. ID No. 722, Seq. ID No. 724, Seq. ID No. 748, Seq. ID No. 752, Seq. ID No. 756, Seq. ID No. 758, Seq. ID No. 760, Seq. ID No. 762, Seq. ID No. 764, Seq. ID No. 770, Seq. ID No. 778, Seq. ID No. 780, Seq. ID No. 808, Seq. ID No. 810, Seq. ID No. 820, Seq. ID No. 826, Seq. ID No. 864, Seq. ID No. 894, Seq. ID No. 902, Seq. ID No. 936, Seq. ID No. 944, Seq. ID No. 956, Seq. ID No. 968, Seq. ID No. 980, Seq. ID No. 1006, Seq. ID No. 1010, Seq. ID No. 1012, Seq. ID No. 1014, Seq. ID No. 1016, Seq. ID No. 1020, Seq. ID No. 1030, Seq. ID No. 1032, Seq. ID No. 1034, Seq. ID No. 1038, Seq. ID No. 1040, Seq. ID No. 1046, Seq. ID No. 1056, Seq. ID No. 1076, Seq. ID No. 1106, Seq. ID No. 1132, Seq. ID No. 1136, Seq. ID No. 1138, Seq. ID No. 1166, Seq. ID No. 1168, Seq. ID No. 1170, Seq. ID No. 1184, Seq. ID No. 1194, Seq. ID No. 1198, Seq. ID No. 1214, Seq. ID No. 1224, Seq. ID No. 1228, Seq. ID No. 1230, Seq. ID No. 1238, Seq. ID No. 1244, Seq. ID No. 1266, Seq. ID No. 1268, Seq. ID No. 1270, Seq. ID No. 1272, Seq. ID No. 1284, Seq. ID No. 1288, Seq. ID No. 1292, Seq. ID No. 1298, Seq. ID No. 1306, Seq. ID No. 1316, Seq. ID No. 1326, Seq. ID No. 1328, Seq. ID No. 1330, Seq. ID No. 1332, Seq. ID No. 1342, Seq. ID No. 1354, Seq. ID No. 1368, Seq. ID No. 1402, Seq. ID No. 1430, Seq. ID No. 1436, Seq. ID No. 1446, Seq. ID No. 1482, Seq. ID No. 1514, Seq. ID No. 1516, Seq. ID No. 1518, Seq. ID No. 1530, Seq. ID No. 1534, Seq. ID No. 1536, Seq. ID No. 1546, Seq. ID No. 1548, Seq. ID No. 1552, Seq. ID No. 1560, Seq. ID No. 1564, Seq. ID No. 1572, Seq. ID No. 1580, Seq. ID No. 1584, Seq. ID No. 1586, Seq. ID No. 1622, Seq. ID No. 1634, Seq. ID No. 1644, Seq. ID No. 1646, Seq. ID No. 1648, Seq. ID No. 1650, Seq. ID No. 1654, Seq. ID No. 1672, Seq. ID No. 1674, Seq. ID No. 1678, Seq. ID No. 1682, Seq. ID No. 1708, Seq. ID No. 1732, Seq. ID No. 1734, Seq. ID No. 1738, Seq. ID No. 1754, Seq. ID No. 1758, Seq. ID No. 1766, Seq. ID No. 1768, Seq. ID No. 1788, Seq. ID No. 1792, Seq. ID No. 1798, Seq. ID No. 1806, Seq. ID No. 1820, Seq. ID No. 1822, Seq. ID No. 1824, Seq. ID No. 1840, Seq. ID No. 1870, Seq. ID No. 1876, Seq. ID No. 1892, Seq. ID No. 1894, Seq. ID No. 1896, Seq. ID No. 1924, Seq. ID No. 1928, Seq. ID No. 1970, Seq. ID No. 1982, Seq. ID No. 1992, Seq. ID No. 1994, Seq. ID No. 2014, Seq. ID No. 2018, Seq. ID No. 2028, Seq. ID No. 2034, Seq. ID No. 2042, Seq. ID No. 2050, Seq. ID No. 2054, Seq. ID No. 2088, Seq. ID No. 2090, Seq. ID No. 2106, Seq. ID No. 2116, Seq. ID No. 2122, Seq. ID No. 2128, Seq. ID No. 2152, Seq. ID No. 2160, Seq. ID No. 2172, Seq. ID No. 2174, Seq. ID No. 2178, Seq. ID No. 2188, Seq. ID No. 2238, Seq. ID No. 2240, Seq. ID No. 2246, Seq. ID No. 2250, Seq. ID No. 2272, Seq. ID No. 2284, Seq. ID No. 2290, Seq. ID No. 2292, Seq. ID No. 2294, Seq. ID No. 2296, Seq. ID No. 2304, Seq. ID No. 2306, Seq. ID No. 2308, Seq. ID No. 2318, Seq. ID No. 2320, Seq. ID No. 2322, Seq. ID No. 2324, Seq. ID No. 2326, Seq. ID No. 2328, Seq. ID No. 2334, Seq. ID No. 2344, Seq. ID No. 2348, Seq. ID No. 2350, Seq. ID No. 2352, Seq. ID No. 2354, Seq. ID No. 2356, Seq. ID No. 2358, Seq. ID No. 2366, Seq. ID No. 2390, Seq. ID No. 2392, Seq. ID No. 2408, Seq. ID No. 2420, Seq. ID No. 2422, Seq. ID No. 2428, Seq. ID No. 2430, Seq. ID No. 2434, Seq. ID No. 2450, Seq. ID No. 2460, Seq. ID No. 2490, Seq. ID No. 2498, Seq. ID No. 2504, Seq. ID No. 2508, Seq. ID No. 2516, Seq. ID No. 2524, Seq. ID No. 2526, Seq. ID No. 2532, Seq. ID No. 2536, Seq. ID No. 2538, Seq. ID No. 2542, Seq. ID No. 2544, Seq. ID No. 2546, Seq. ID No. 2556, Seq. ID No. 2558, Seq. ID No. 2570, Seq. ID No. 2572, Seq. ID No. 2584, Seq. ID No. 2592, Seq. ID No. 2598, Seq. ID No. 2600, Seq. ID No. 2602, Seq. ID No. 2608, Seq. ID No. 2612, Seq. ID No. 2622, Seq. ID No. 2630, Seq. ID No. 2652, Seq. ID No. 2680, Seq. ID No. 2694, Seq. ID No. 2702, Seq. ID No. 2704, Seq. ID No. 2706, Seq. ID No. 2708, Seq. ID No. 2732, Seq. ID No. 2734, Seq. ID No. 2784, Seq. ID No. 2786, Seq. ID No. 2814, Seq. ID No. 2816, Seq. ID No. 2824, Seq. ID No. 2836, Seq. ID No. 2838, Seq. ID No. 2842, Seq. ID No. 2854, Seq. ID No. 2874, Seq. ID No. 2876, Seq. ID No. 2882, Seq. ID-No. 2906, Seq. ID No. 2936, Seq. ID No. 2938, Seq. ID No. 2940, Seq. ID No. 2942, Seq. ID No. 2948, Seq. ID No. 2950, Seq. ID No. 2970, Seq. ID No. 2974, Seq. ID No. 2978, Seq. ID No. 2982, Seq. ID No. 2988, Seq. ID No. 2992, Seq. ID No. 2996, Seq. ID No. 2998, Seq. ID No. 3006, Seq. ID No. 3008, Seq. ID No. 3014, Seq. ID No. 3024, Seq. ID No. 3026, Seq. ID No. 3028, Seq. ID No. 3030, Seq. ID No. 3032, Seq. ID No. 3044, Seq. ID No. 3056, Seq. ID No. 3064, Seq. ID No. 3072, Seq. ID No. 3074, Seq. ID No. 3076, Seq. ID No. 3078, Seq. ID No. 3124, Seq. ID No. 3146, Seq. ID No. 3156, Seq. ID No. 3158, Seq. ID No. 3164, Seq. ID No. 3184, Seq. ID No. 3194, Seq. ID No. 3202, Seq. ID No. 3214, Seq. ID No. 3216, Seq. ID No. 3218, Seq. ID No. 3220, Seq. ID No. 3242, Seq. ID No. 3244, Seq. ID No. 3246, Seq. ID No. 3250, Seq. ID No. 3258, Seq. ID No. 3262, Seq. ID No. 3270, Seq. ID No. 3276, Seq. ID No. 3298, Seq. ID No. 3318, Seq. ID No. 3324, Seq. ID No. 3338, Seq. ID No. 3340, Seq. ID No. 3350, Seq. ID No. 3352, Seq. ID No. 3354, Seq. ID No. 3358, Seq. ID No. 3360, Seq. ID No. 3378, Seq. ID No. 3390, Seq. ID No. 3392, Seq. ID No. 3396, Seq. ID No. 3406, Seq. ID No. 3422, Seq. ID No. 3426, Seq. ID No. 3450, Seq. ID No. 3452, Seq. ID No. 3454, Seq. ID No. 3456, Seq. ID No. 3458, Seq. ID No. 3464, Seq. ID No. 3482, Seq. ID No. 3492, Seq. ID No. 3494, Seq. ID No. 3504, Seq. ID No. 3506, Seq. ID No. 3528, Seq. ID No. 3594, Seq. ID No. 3596, Seq. ID No. 3622, Seq. ID No. 3626, Seq. ID No. 3636, Seq. ID No. 3680, Seq. ID No. 3724, Seq. ID No. 3728, Seq. ID No. 3746, Seq. ID No. 3754, Seq. ID No. 3756, Seq. ID No. 3758, Seq. ID No. 3762, Seq. ID No. 3764, Seq. ID No. 3766, Seq. ID No. 3784, Seq. ID No. 3794, Seq. ID No. 3800, Seq. ID No. 3814, Seq. ID No. 3838, Seq. ID No. 3840, Seq. ID No. 3850, Seq. ID No. 3852, Seq. ID No. 3854, Seq. ID No. 3860, Seq. ID No. 3862, Seq. ID No. 3864, Seq. ID No. 3866, Seq. ID No. 3880, Seq. ID No. 3890, Seq. ID No. 3892, Seq. ID No. 3904, Seq. ID No. 3926, Seq. ID No. 3964, Seq. ID No. 3974, Seq. ID No. 3976, Seq. ID No. 4006, Seq. ID No. 4026, Seq. ID No. 4028, Seq. ID No. 4040, Seq. ID No. 4042, Seq. ID No. 4044, Seq. ID No. 4046, Seq. ID No. 4048, Seq. ID No. 4062, Seq. ID No. 4072, Seq. ID No. 4074, Seq. ID No. 4080, Seq. ID No. 4082, Seq. ID No. 4084, Seq. ID No. 4102, Seq. ID No. 4104, Seq. ID No. 4112, Seq. ID No. 4134, Seq. ID No. 4138, Seq. ID No. 4140, Seq. ID No. 4146, Seq. ID No. 4152, Seq. ID No. 4222, Seq. ID No. 4224, Seq. ID No. 4226, Seq. ID No. 4228, Seq. ID No. 4232, Seq. ID No. 4242, Seq. ID No. 4248, Seq. ID No. 4254, Seq. ID No. 4258, Seq. ID No. 4262, Seq. ID No. 4308, Seq. ID No. 4312, Seq. ID No. 4314, Seq. ID No. 4344, Seq. ID No. 4380, Seq. ID No. 4386, Seq. ID No. 4390, Seq. ID No. 4440, Seq. ID No. 4470, Seq. ID No. 4474, Seq. ID No. 4482, Seq. ID No. 4484, Seq. ID No. 4486, Seq. ID No. 4498, Seq. ID No. 4516, Seq. ID No. 4534, Seq. ID No. 4588, Seq. ID No. 4640, Seq. ID No. 4644, Seq. ID No. 4682, Seq. ID No. 4706, Seq. ID No. 4708, Seq. ID No. 4710, Seq. ID No. 4712, Seq. ID No. 4716, Seq. ID No. 4718, Seq. ID No. 4738, Seq. ID No. 4760, Seq. ID No. 4814, Seq. ID No. 4828, Seq. ID No. 4870, Seq. ID No. 4872, Seq. ID No. 4874, Seq. ID No. 4876, Seq. ID No. 4920, Seq. ID No. 4932, Seq. ID No. 4950, Seq. ID No. 4952, Seq. ID No. 4988, Seq. ID No. 5032, Seq. ID No. 5062, Seq. ID No. 5086, Seq. ID No. 5104, Seq. ID No. 5124, Seq. ID No. 5130, Seq. ID No. 5132, Seq. ID No. 5136, Seq. ID No. 5138, Seq. ID No. 5142, Seq. ID No. 5166, Seq. ID No. 5176, Seq. ID No. 5200, Seq. ID No. 5206, Seq. ID No. 5208, Seq. ID No. 5214, Seq. ID No. 5220, Seq. ID No. 5224, Seq. ID No. 5268, Seq. ID No. 5282, Seq. ID No. 5386, Seq. ID No. 5388, Seq. ID No. 5414, Seq. ID No. 5416, Seq. ID No. 5434, Seq. ID No. 5454, Seq. ID No. 5460, Seq. ID No. 5462, Seq. ID No. 5464, Seq. ID No. 5466, Seq. ID No. 5492, Seq. ID No. 5498, Seq. ID No. 5510, Seq. ID No. 5514, Seq. ID No. 5526, Seq. ID No. 5532, Seq. ID No. 5570, Seq. ID No. 5588, Seq. ID No. 5596, Seq. ID No. 5598, Seq. ID No. 5610, Seq. ID No. 5612, Seq. ID No. 5622, Seq. ID No. 5624, Seq. ID No. 5634, Seq. ID No. 5638, Seq. ID No. 5644, Seq. ID No. 5650, Seq. ID No. 5660, Seq. ID No. 5684, Seq. ID No. 5686, Seq. ID No. 5700, Seq. ID No. 5706, Seq. ID No. 5710, Seq. ID No. 5730, Seq. ID No. 5734, Seq. ID No. 5764, Seq. ID No. 5766, Seq. ID No. 5768, Seq. ID No. 5772, Seq. ID No. 5784, Seq. ID No. 5792, Seq. ID No. 5798, Seq. ID No. 5886, Seq. ID No. 5890, Seq. ID No. 5894, Seq. ID No. 5896, Seq. ID No. 5900, Seq. ID No. 5902, Seq. ID No. 5906, Seq. ID No. 5910, Seq. ID No. 5922, Seq. ID No. 5924, Seq. ID No. 5928, Seq. ID No. 5940, Seq. ID No. 6028, Seq. ID No. 6044, Seq. ID No. 6048, Seq. ID No. 6060, Seq. ID No. 6068, Seq. ID No. 6090, Seq. ID No. 6092, Seq. ID No. 6108, Seq. ID No. 6112, Seq. ID No. 6118, Seq. ID No. 6162, Seq. ID No. 6164, Seq. ID No. 6166, Seq. ID No. 6170, Seq. ID No. 6184, Seq. ID No. 6186, Seq. ID No. 6190, Seq. ID No. 6192, Seq. ID No. 6198, Seq. ID No. 6204, Seq. ID No. 6206, Seq. ID No. 6210, Seq. ID No. 6222, Seq. ID No. 6224, Seq. ID No. 6226, Seq. ID No. 6228, Seq. ID No. 6230, Seq. ID No. 6236, Seq. ID No. 6238, Seq. ID No. 6244, Seq. ID No. 6246, Seq. ID No. 6250, Seq. ID No. 6254, Seq. ID No. 6264, Seq. ID No. 6266, Seq. ID No. 6274, Seq. ID No. 6276, Seq. ID No. 6286, Seq. ID No. 6290, Seq. ID No. 6302, Seq. ID No. 6304, Seq. ID No. 6364, Seq. ID No. 6378, Seq. ID No. 6382, Seq. ID No. 6384, Seq. ID No. 6416, Seq. ID No. 6428, Seq. ID No. 6432, Seq. ID No. 6474, Seq. ID No. 6498, Seq. ID No. 6572, Seq. ID No. 6582, Seq. ID No. 6594, Seq. ID No. 6600, Seq. ID No. 6632, and Seq. ID No. 6640.

In another aspect of the invention, the polypeptides are expressed and purified in a recombinant expression system. Thus, in certain embodiments, the invention provides a recombinant expression vector comprising a nucleotide sequence having at least about 70% identity to a nucleotide sequence chosen from one of odd numbered sequences set out in SEQ ID NO: 1 through SEQ ID NO: 6649, a complement thereof, a degenerate variant thereof, or a fragment thereof.

In certain other embodiments, the polynucleotide is selected from the group consisting of genomic DNA, cDNA, and RNA. In another embodiment, the polynucleotide comprised within the vector further comprises heterologous nucleotide sequences.

In other embodiments, the polynucleotide is operatively linked to one or more gene expression regulatory elements. In yet other embodiments, the polynucleotide encodes a polypeptide comprising an amino acid sequence having at least about 70% identity to an amino acid sequence chosen from one of even numbered sequences set out in SEQ ID NO: 2 through SEQ ID NO: 6650, a complement thereof, a biological equivalent thereof, or a fragment thereof. In a preferred embodiment, the vector is a plasmid.

In another aspect of the invention, provided is a genetically engineered host cell, transfected, transformed or infected with a recombinant expression vector comprising a nucleotide sequence having at least about 70% identity to a nucleotide sequence chosen from one of odd numbered sequences set out in SEQ ID NO: 1 through SEQ ID NO: 6649, a complement thereof, a degenerate variant thereof, or a fragment thereof. In a preferred embodiment, the host cell is a bacterial cell. In a further embodiment, the polynucleotide is expressed to produce the encoded polypeptide, a complement thereof, a biological equivalent thereof, or a fragment thereof.

In another aspect of the invention, the polypeptides are expressed and purified in a recombinant expression system. Thus, in certain embodiments, the invention provides a recombinant expression vector comprising a nucleotide sequence having at least about 70% identity to a nucleotide sequence chosen from one of odd numbered sequences set out in SEQ ID NO: 1 through SEQ ID NO: 6649, a complement thereof, a degenerate variant thereof, or a fragment thereof. In certain other embodiments, the polynucleotide is selected from the group consisting of genomic DNA, cDNA, and RNA. In another embodiment, the polynucleotide comprised within the vector further comprises heterologous nucleotide sequences.

In other embodiments, the present invention provides an antibody specific for an Alloiococcus otitidis polynucleotide chosen from one of odd numbered sequences set out in SEQ ID NO: 1 through SEQ ID NO: 6649, a fragment thereof, a degenerate variant thereof, or an antibody specific for an Alloiococcus otitidis polypeptide chosen from one of even numbered sequences set out in SEQ ID NO: 2 through SEQ ID NO: 6650, a biological equivalent thereof, or a fragment thereof. In certain embodiments, the antibody is selected from the group consisting of monoclonal, polyclonal, chimeric, human, humanized and single chain. In a preferred embodiment, the antibody is monoclonal. In another preferred embodiment, the antibody is humanized.

The present invention further provides pharmaceutical compositions, in particular immunogenic compositions, for the prevention and/or treatment of bacterial infection. Thus, in one embodiment an immunogenic composition is provided comprising one or more polypeptides having an amino acid sequence chosen from one or more of even numbered sequences set out in SEQ ID NO: 2 through SEQ ID NO: 6650, a biological equivalent thereof, or a fragment thereof. In certain embodiments, the composition further comprises a pharmaceutically acceptable carrier. In yet other embodiments, the immunogenic composition further comprises one or more adjuvants. In preferred embodiments the polypeptide of the invention is further defined as: (a) an Alloiococcus otitidis polypeptide predicted by SignalP algorithm analysis as having met all four criteria set out in the SignalP algorithm for a polypeptide having a signal peptide and a subset of these are predicted by HMM SignalP algorithm analysis as having a signal peptide; (b) an Alloiococcus otitidis polypeptide predicted by SignalP algorithm analysis as having met three of the four criteria set out in the SignalP algorithm for a polypeptide having a signal peptide and a subset of these predicted by HMM SignalP algorithm analysis as having a signal peptide; (c) an Alloiococcus otitidis polypeptide predicted by HMM SignalP algorithm analysis as having a signal peptide; (d) an Alloiococcus otitidis polypeptide predicted by HMM SignalP algorithm analysis as being a non-secretory protein; (e) an Alloiococcus otitidis polypeptide identified by Pfam analysis; (f) an Alloiococcus otitidis polypeptide identified by BlastP analysis as being secreted, surface exposed, vaccine candidate; (g) an Alloiococcus otitidis polypeptide identified using proteomics methodology as sharing homology with surface exposed proteins of Streptococcus pneumoniae; (h) an Alloiococcus otitidis polypeptide having a LPXTG motif, wherein the polypeptide is covalently attached to the peptidoglycan layer; (i) an Alloiococcus otitidis lipoprotein; (j) an Alloiococcus otitidis polypeptide non-covalently associated with the peptidoglycan layer; (k) an Alloiococcus otitidis polypeptide having an RGDX motif wherein X is not a proline residue; (l) an Alloiococcus otitidis polypeptide predicted by BlastP as being involved in capsule biosynthesis and transport; (m) an Alloiococcus otitidis polypeptide identified by BlastP as being localized within the capsule loci of the Alloiococcus otitidis genome; (n) an Alloiococcus otitidis polypeptide predicted by BlastP as being associated with sporulation; (o) an Alloiococcus otitidis polypeptide encoded by unique ORFs identified by BlastP analysis as having a BlastP ‘E Value’ of >e⁻¹⁰; (p) an Alloiococcus otitidis polypeptide identified by Glimmer™ ORF finder program; (q) an Alloiococcus otitidis polypeptide identified by GeneMark™ ORF finder program; (r) an Alloiococcus otitidis polypeptide identified by Applicants' assignee's ORF finder program that searches for an ATG, GTG or TTG Start codon between a Stop-Stop region; and (s) an Alloiococcus otitidis polypeptide identified by Applicants' assignee's ORF finder program that searches for a transmembrane domain between two Stop codons and a Start codon immediately upstream of this transmembrane region.

In one preferred embodiment,the invention comprises an isolated polypeptide encoded by a polynucleotide contained-within the Alloiococcus otitidis genomic sequence, wherein the polynucleotide comprises a nucleotide sequence having at least about 70% identity to a nucleotide sequence contained within SEQ ID NO: 6651, a complement thereof, a degenerate variant thereof, and a fragment thereof.

In certain other embodiments, the immunogenic composition further comprises heterologous amino acids. In particular embodiments, the polypeptide is a fusion polypeptide.

In further embodiments, provided is an immunogenic composition comprising a polynucleotide having a nucleotide sequence chosen from one of odd numbered sequences listed in SEQ ID NO: 1 through SEQ ID NO: 6649, a degenerate variant thereof, or a fragment thereof and is comprised in an expression vector. In preferred embodiments, the vector is plasmid DNA. In another embodiment, the polynucleotide comprises heterologous nucleotides. In still other embodiments, the polynucleotide is operatively linked to one or more gene expression regulatory elements. In yet other embodiments, the polynucleotide directs the expression of a neutralizing epitope of Alloiococcus otitidis. In preferred embodiments, the immunogenic composition further comprises one or more adjuvants.

In yet further embodiments, provided is a pharmaceutical composition comprising a polypeptide and a pharmaceutically acceptable carrier, wherein the polypeptide comprises an amino acid chosen from one of even numbered sequence listings set out in SEQ ID NO: 2 through SEQ ID NO: 6650, a complement thereof, a biological equivalent thereof, or a fragment thereof. In preferred embodiments the polypeptide of the invention is further defined as: (a) an Alloiococcus otitidis polypeptide predicted by SignalP algorithm analysis as having met all four criteria set out in the SignalP algorithm for a polypeptide having a signal peptide and a subset of these are predicted by HMM SignalP algorithm analysis as having a signal peptide; (b) an Alloiococcus otitidis polypeptide predicted by SignalP algorithm analysis as having met three of the four criteria set out in the SignalP algorithm for a polypeptide having a signal peptide and a subset of these predicted by HMM SignalP algorithm analysis as having a signal peptide; (c) an Alloiococcus otitidis polypeptide predicted by HMM SignalP algorithm analysis as having a signal peptide; (d) an Alloiococcus otitidis polypeptide predicted by HMM SignalP algorithm analysis as being a non-secretory protein; (e) an Alloiococcus otitidis polypeptide identified by Pfam analysis; (f) an Alloiococcus otitidis polypeptide identified by BlastP analysis as being secreted, surface exposed, vaccine; (g) an Alloiococcus otitidis polypeptide identified using proteomics methodology as sharing homology with surface exposed proteins of Streptococcus pneumoniae; (h) an Alloiococcus otitidis polypeptide having a LPXTG motif, wherein the polypeptide is covalently attached to the peptidoglycan layer; (i) an Alloiococcus otitidis lipoprotein; (j) an Alloiococcus otitidis polypeptide non-covalently associated with the peptidoglycan layer; (k) an Alloiococcus otitidis polypeptide having an RGDX motif wherein X is not a proline residue; (l) an Alloiococcus otitidis polypeptide predicted by BlastP as being involved in capsule biosynthesis and transport; (m) an Alloiococcus otitidis polypeptide identified by BlastP as being localized within the capsule loci of the Alloiococcus otitidis genome; (n) an Alloiococcus otitidis polypeptide predicted by BlastP as being associated with sporulation; (o) an Alloiococcus otitidis polypeptide encoded by unique ORFs identified by BlastP analysis as having a BlastP ‘E Value’ of >e⁻¹⁰; (p) an Alloiococcus otitidis polypeptide identified by Glimmer™ ORF finder program; (q)an Alloiococcus otitidis polypeptide identified by GeneMark™ ORF finder program; (r) an Alloiococcus otitidis polypeptide identified by Applicants' assignee's ORF finder program that searches for an ATG, GTG or TTG start codon between a Stop-Stop region; and (s) an Alloiococcus otitidis polypeptide identified by Applicants' assignee's ORF finder program that searches for a transmembrane domain between two Stop codons and a Start codon immediately upstream of this transmembrane region.

In another embodiment, a method of immunizing against Alloiococcus otitidis is provided comprising administering to a host an immunizing amount of an immunogenic composition comprising a polypeptide and a pharmaceutically acceptable carrier, wherein the polypeptide comprises an amino acid sequence chosen from one of even numbered sequences set forth in SEQ ID NO: 2 through SEQ ID NO: 6650, a biological equivalent thereof, or a fragment thereof. In certain embodiments, the polypeptide is a fusion polypeptide. In other embodiments, the method further comprises administering an adjuvant.

In other embodiments the polypeptides of the invention comprise heterologous amino acids. In still other embodiments, the polypeptide in the pharmaceutical composition is a fusion polypeptide.

Other embodiments of the invention provide a DNA chip comprising an array of polynucleotides, wherein at least one of the polynucleotides comprise a nucleotide sequence chosen from one of odd numbered sequences set forth in SEQ ID NO: 1 through SEQ ID NO: 6649, a complement thereof, a degenerate variant thereof, or a fragment thereof.

Also provided is a protein chip comprising an array of polypeptides, wherein at least one of the polypeptides comprises an amino acid sequence chosen from one of even numbered sequences set forth in SEQ ID NO: 2 through SEQ ID NO: 6650, a biological equivalent thereof, or a fragment thereof.

The invention further provides methods of detecting Alloiococcus otitidis polynucleotides and polypeptides as well as kits for diagnosing Alloiococcus otitidis infection. Other embodiments provide a method for the detection and/or identification of Alloiococcus otitidis in a biological sample comprising contacting the sample with an oligonucleotide probe of a polynucleotide comprising the nucleotide sequence chosen from one of odd numbered sequences is set forth in SEQ ID NO: 1 through SEQ ID NO: 6649, a degenerate variant thereof, or a fragment thereof, under conditions permitting hybridization and detecting the presence of hybridization complexes in the sample, wherein hybridization complexes indicate the presence of Alloiococcus otitidis in the sample.

Still other embodiments provide a method for the detection and/or identification of Alloiococcus otitidis in a biological sample comprising nucleotide sequence chosen from one of odd numbered sequences set forth in SEQ ID NO: 1 through SEQ ID NO: 6649, a degenerate variant thereof, or a fragment thereof, in the presence of nucleotides and a polymerase enzyme under conditions permitting primer extension and detecting the presence of primer extension products in the sample, wherein extension products indicate the presence of Alloiococcus otitidis in the sample.

Further embodiments provide a method for the detection and/or identification of Alloiococcus otitidis in a biological sample comprising contacting the sample with an antibody specific for a polypeptide comprising an amino acid sequence chosen from one of even numbered sequences set forth in SEQ ID NO: 2 through SEQ ID NO: 6650, a biological equivalent thereof, or a fragment thereof, under conditions permitting immune complex formation and detecting the presence of immune complexes in the sample, wherein immune complexes indicate the presence of Alloiococcus otitidis in the sample.

In certain embodiments, provided is a method for the detection and/or identification of antibodies to Alloiococcus otitidis in a biological sample comprising contacting the sample with a polypeptide comprising an amino acid sequence chosen from one of even numbered sequences set forth in SEQ ID NO: 2 through SEQ ID NO: 6650, a biological equivalent thereof, or a fragment thereof, under conditions permitting immune complex formation and detecting the presence of immune complexes in the sample, wherein immune complexes indicate the presence of Alloiococcus otitidis in the sample.

Other embodiments of the invention provide a kit comprising a container containing an isolated polynucleotide comprising an nucleotide sequence chosen from one of odd numbered sequences set forth in SEQ ID NO: 1 through SEQ ID NO: 6649, a degenerate variant thereof, or a fragment thereof. In a preferred embodiment, the polynucleotide is a primer or a probe, wherein when the polynucleotide is a primer, the kit further comprises a container containing a polymerase. In another embodiment, the kit further comprises a container containing dNTP.

Provided further is a kit comprising a container containing an antibody that immunospecifically binds to a polypeptide comprising the amino acid sequence chosen from one of even numbered sequences set forth in SEQ ID NO: 2 through SEQ ID NO: 6650, a biological equivalent thereof, or a fragment thereof.

Provided also is a kit comprising a container containing an antibody that immunospecifically binds to a fusion polypeptide comprising at least the amino acid sequence chosen from one of even numbered sequences set forth in SEQ ID NO: 2 through SEQ ID NO: 6650, a biological equivalent thereof, or a fragment thereof.

In a preferred embodiment of the invention, there is provided a genetically engineered host cell, transfected, transformed or infected with a recombinant expression vector comprising a nucleotide sequence having at least about 70% identity to a nucleotide sequence chosen from one of odd numbered sequences set forth in SEQ ID NO: 1 through SEQ ID NO: 6649, a complement thereof, a degenerate variant thereof, or a fragment thereof under conditions suitable to produce one of the polypeptides of even numbered sequences set out in SEQ ID NO: 2 through SEQ ID NO: 6650; and recovering the polypeptide.

Other features and advantages of the invention will be apparent from the following detailed description, from the preferred embodiments thereof, and from the claims.

### DETAILED DESCRIPTION OF THE INVENTION

The invention described herein addresses the need for an Alloiococcus otitidis immunogenic composition that is effective in preventing most or all of the disease caused by Alloiococcus otitidis. The invention further addresses the need for methods of diagnosing Alloiococcus otitidis infection. The Applicants have identified novel Alloiococcus otitidis open reading frames (ORFs), which encode antigenic polypeptides. More particularly, the newly identified ORFs encode polypeptides that are surface localized (i.e., exposed) on Alloiococcus otitidis, and thus serve as potential antigenic polypeptides in immunogenic compositions. Thus, in certain embodiments, the invention comprises Alloiococcus otitidis polynucleotide ORFs encoding surface localized polypeptide antigens. In other embodiments, the present invention comprises surface localized antigenic polypeptides, encoded by the Alloiococcus otitidis ORFs.

In other embodiments, the invention comprises vectors comprising ORF sequences and host cells or animals transformed with these vectors. The invention also comprises transcriptional gene products of Alloiococcus otitidis ORFs, including, but not limited to mRNA, antisense RNA, antisense oligonucleotides, and ribozyme molecules, which are used to inhibit or control growth of the microorganism. The invention relates also to methods of detecting Alloiococcus otitidis nucleic acids or polypeptides and kits for diagnosing Alloiococcus otitidis infection. The invention also relates to pharmaceutical compositions, in particular immunogenic compositions, for the prevention and/or treatment of bacterial infection, in particular infection caused by or exacerbated by Alloiococcus otitidis. In particular embodiments, the immunogenic compositions are used for the treatment or prevention of non-systemic diseases, particularly of the otitis media, which are induced or exacerbated by Alloiococcus otitidis.

### A. IDENTIFYING ORFs WITHIN THE GENOMIC SEQUENCE OF ALLOIOCOCCUS OTITIDIS

The invention is directed in particular embodiments to the identification of polynucleotides, more particularly ORFs, which encode Alloiococcus otitidis polypeptides. The availability of the bacterial genomic sequences has begun to play an important role in the identification of vaccine candidates through genomics, transcriptional profiling, and proteomics, coupled with the information processing capabilities of bioinformatics (McAtee et al., 1998a; McAtee et al., 1998b; Pizza et al., 2000; Sonnenberg and Belisle, 1997; Weldingh et al., 1998; McAtee et al., 1998c). Thus, the Applicants have analyzed the Alloiococcus otitidis genomic sequences and utilized bioinformatics tools to identify the ORFs encoding polypeptides of the present invention. As described below, the ORFs were analyzed for a variety of characteristics.

Specifically, an extensive genomic analysis was performed in silico of the Alloiococcus otitidis genome using algorithms designed to identify genes that encode novel surface localized polypeptides or polypeptides with putative similarity to surface localized polypeptides in other organisms. Thus, a combined analysis of the Alloiococcus otitidis genome, using a unique set of three ORF finder algorithms (i.e., GLIMMER™, Salzberg et al., 1998, GeneMark™ (Lukashin and Borodovsky, 1998) and the Applicants' assignee's own programs), identified 3325 ORFs. The most stringent of the ORF finders; Glimmer™, identified 1529 ORFs; GeneMark™ identified 1,534 ORFs, while Applicants' assignee's ORF finders identified the most with 2706 ORFs.

For the purposes of this application, all ORFs with common stop codons are given the same ORF designation and will be treated as if they are the same ORF. In a number of instances, there are multiple start codons within a given ORF. These proteins of different length are indicated by a lower case letter suffix after the ORF number. For example, as shown in Table 2 below, ORF number 4a has a protein start beginning at nucleotide 3733, ORF number 4b has a protein start beginning at nucleotide 3049, and 4c has a protein start beginning or ending at nucleotide 2854 (these three sequences share a common ORF stop codon beginning at nucleotide 4152). As used herein, an ORF is defined as having one of three potential start site codons, ATG, GTG, TTG and one of three potential stop codons, TM, TAG, TGA. The lower limit of amino acid length used (e.g., ˜74 amino acids) may also cause the algorithms to overlook some reading frames. However, “true” reading frames become an increasingly rare as the ORFs become shorter.

The initial annotation of the Alloiococcus otitidis ORFs was performed using the Basic Local Alignment Search Tool (BLAST; version 2.0) Gapped search algorithm, BlastP, to identify homologous sequences (Altschul et al, 1997). A cutoff “E” value of anything <e⁻¹⁰ was considered significant. The non-redundant protein sequence databases used for the homology searches consisted of GenBank, SWISS-PROT, PIR, and TREMBL; these database sequences are updated daily. In the present invention, ORFs with a BlastP ‘E’ value result of >e⁻¹⁰ are considered to be unique to Alloiococcus otitidis. Alternate quantitative expression values other than BlastP “E”, e.g., percent identity, may also be used to compare database sequences with the Alloiococcus otitidis ORFs of the present invention.

A keyword search of the entire BLAST results was carried out using known or suspected target genes for immunogenic compositions, as well as using words that identified the location of a protein or function.

Several parameters were used to determine the partitioning of the predicted Alloiococcus otitidis polypeptides of the invention. For example, polypeptides destined for translocation across the cytoplasmic membrane encode a leader signal (also called signal sequence) composed of a central hydrophobic region flanked at the N-terminus by positively charged residues (Pugsley, 1993). A software program, called SignalP, which identifies signal peptides and their cleavage sites based on neural networks (Nielsen et al., 1997), was used in the present invention to analyze the amino acid sequence of an ORF for such signal peptides. The first 60 N-terminal amino acids of each ORF were analyzed by SignalP software using the Gram-positive software database. The output generated four separate values: maximum C, maximum Y, maximum S, and mean S. The S-score, or signal region, is the probability of the position belonging to the signal peptide. The C-score, or cleavage site, is the probability of the position being the first in the mature protein. The Y-score is the geometric average of the C-score and a smoothed derivative of the S-score. A conclusion of either a Yes or No is given next to each score. If all four conclusions are Yes, then a ‘YES’ is listed for that ORF (see Table 3 below); if three of the conclusions are Yes, then a ‘yes’ is listed for that ORF (see Table 4 below); if two of the conclusions are Yes, then a ‘maybe’ is listed for that ORF; for all other cases, a ‘No’ is listed for that ORF (see Table 5 below).

A software program, called HMM SignalP, which identifies signal peptides and their cleavage sites based on hidden Markov models (Nielsen et al., 1997), was used in the present invention to analyze the amino acid sequence of an ORF for such signal peptides. The first 60 N-terminal amino acids of each ORF were analyzed by HMM SignalP software using the Gram-positive software database. The output generates a value from 0.00 to 1.00, whereby any value greater than 0.5 is listed as being a ‘Signal Peptide’ (see Table 5 below) and any value less than 0.5 is listed as being a ‘Non-Secretory Protein’ (see Table 6 below).

To predict polypeptide localization in bacteria, the software program PSORT was used (Nakai, 1991). PSORT predicts localization of polypeptides to the ‘cytoplasm’, the ‘periplasm’, and/or the ‘cytoplasmic membrane’ for Gram-positive bacteria, as well as the ‘outer membrane’ for Gram-negative bacteria. Transmembrane (TM) domains of polypeptides were analyzed using the software program TopPred2 (Cserzo et al., 1997) (see Table 6 below).

The Hidden Markov Model (HMM) Pfam database (Sonnhammer, 1998) was used to identify Alloiococcus otitidis proteins that may belong to an existing protein family (see Table 7 below). Keyword searching of this output was further used to help identify additional vaccine candidates that may have been missed by the BLAST search criteria (see Table 8 below).

The Applicants also used proteomic methods that had previously been used for studying surface exposed proteins in Streptococcus pneumoniae to identify 242 ORFs of Alloiococcus otitidis that share some homology with ORFs encoding surface exposed proteins on Streptococcus pneumoniae (see Table 9).

The Applicants also developed a HMM using approximately 70 known prokaryotic proteins containing the LPXTG cell wall sorting signal, in order to predict cell wall proteins that are anchored to the peptidoglycan layer (Mazmanian et al., 1999; Navarre and Schneewind, 1999). The model used not only the LPXTG sequence, but also included two features of the downstream sequence; first the hydrophobic transmembrane domain and second, the positively charged carboxy-terminus (see Table 10).

A computer algorithm, called HMM Lipo, was developed by Applicant's assignee to predict lipoproteins using 131 biologically proven bacterial lipoproteins. The protein sequence from the start of the protein to the cysteine amino acid, plus the next two additional amino acids, were used to generate the HMM (Eddy, 1996) (see Table 11).

There are also a number of proteins that interact, non-covalently, with the peptidoglycan layer and are distinct from the LPXTG protein class described above. These proteins seem to have a consensus sequence at their carboxy terminus (Koebnik, 1995). The Applicants therefore developed and used a HMM of this region to identify any Alloiococcus otitidis genome-encoded proteins that may fall into this class of proteins (see Table 12).

Proteins that contain Arg-Gly-Asp (RGD) attachment motif, together with integrins that serve as their receptor, constitute a major recognition system for cell adhesion, and thus are putative Alloiococus otitidis cell surface antigens. RGD recognition is one mechanism used by microbes to gain entry into eukaryotic tissues (Stockbauer et al., 1999, Isberg and Nhieu, 1994). However, not all RGD-containing proteins mediate cell attachment. It has been demonstrated that RGD-containing peptides with a proline at the carboxy end (RDGP) are inactive in cell attachment assays (Pierschbacher and Rouslahti, 1987) and will be excluded from consideration. About 125 surface localized ORFs were identified that were found to contain the peptide sequence RDGX where X is not proline (see Table 13).

ORFs whose BlastP results predict for proteins that may be involved in capsule biosynthesis and transport were also identified based on BlastP analysis. In addition, three potential capsule loci within the Alloiococcus otitidis genome were also identified based BlastP analysis (see Tables 14 and 15).

BlastP analysis was also used to identify ORFs associated with sporulation in Alloiococcus otitidis (see Tables 16).

BlastP analysis of the Alloiococcus otitidis genomic sequence also resulted in the identification of 913 unique ORFs, or 653 unique Stop-Stop ORFs, that were shown to have a BlastP ‘E Value’ of >e⁻¹⁰ (see Table 17).

Three different ORF finder programs were also used to identify ORFs encoding polypeptides that could potentially be used in immunogenic compositions. Using the Glimmer™ ORF finder program 1530 ORFs were identified (see Table 18). Using the GeneMark™ ORF finder program 1536 ORFs were identified (see Table 19). The Applicants also identified 2343 ORFs using the ORF finder program of Applicant's assignee, which searches for one of the three potential Start codons between a Stop-Stop regions. It first looks for a ‘ATG’ Start codon but will accept either a ‘GTG’ or ‘TTG’ Start codon if found first (see Table 20). The Applicants' assignee's ORF finder program also detected 615 ORFs containing putative transmembrane regions The program searches for putative transmembrane regions between two Stop codons and a Start codon immediately upstream of this region (see Table 21).

The entire genomic sequence of the isolated Alloiococcus otitidis is set out in Seq. ID No. 6651. Alloiococcus otitidis ORFs encoding proteins/polypeptides that are contained within this genomic sequence can also be used in immunogenic compositions.

A complete listing of all the ORFs of Alloiococcus otitidis based on the above characteristics, and which are used in immunogenic compositions, is set out in Table 2.

In addition, Tables 3-21 represent 19 groups into which the ORFs identified according to the above characteristics of present invention have been classified. Thus, all of the groups described below are ORFs comprised within the Alloiococcus otitidis genome and identified as encoding putative surface localized polypeptides. These groups are not meant to limit the scope of the present invention, as analysis of additional ORF characteristics is contemplated. These additional characteristics, e.g., RGD sequence, may serve to further expand the total number of ORF groupings or to parse the presently identified ORFs into more defined groups, broader groups, narrower groups or group subsets. In addition, some ORFs will meet the criteria of more than one category, and will therefore appear in more than one of the following groups.

TABLE 2
ALL CLAIMED ORFs

| ORF | DNA SEQ ID | | | | Protein SEQ ID |
|---|---|---|---|---|---|
| Number | Number | ORF Start | Protein Start | ORF Stop | Number |
| 1 | Seq. ID No. 1 | 1149 | 1146 | 280 | Seq. ID No. 2 |
| 2a | Seq. ID No. 3 | 2227 | 2143 | 1271 | Seq. ID No. 4 |
| 2b | Seq. ID No. 5 | 2227 | 2203 | 1271 | Seq. ID No. 6 |
| 4a | Seq. ID No. 7 | 2827 | 3733 | 4152 | Seq. ID No. 8 |
| 4b | Seq. ID No. 9 | 2827 | 3049 | 4152 | Seq. ID No. 10 |
| 4c | Seq. ID No. 11 | 2827 | 2854 | 4152 | Seq. ID No. 12 |
| 5 | Seq. ID No. 13 | 5020 | 5011 | 4295 | Seq. ID No. 14 |
| 7 | Seq. ID No. 15 | 5792 | 5780 | 5001 | Seq. ID No. 16 |
| 10 | Seq. ID No. 17 | 7828 | 7807 | 5783 | Seq. ID No. 18 |
| 11 | Seq. ID No. 19 | 6198 | 6216 | 6449 | Seq. ID No. 20 |
| 13a | Seq. ID No. 21 | 6900 | 6879 | 6589 | Seq. ID No. 22 |
| 16 | Seq. ID No. 23 | 7100 | 7139 | 7390 | Seq. ID No. 24 |
| 17 | Seq. ID No. 25 | 8265 | 8247 | 7804 | Seq. ID No. 26 |
| 18 | Seq. ID No. 27 | 9571 | 9517 | 8210 | Seq. ID No. 28 |
| 20 | Seq. ID No. 29 | 10605 | 10605 | 9514 | Seq. ID No. 30 |
| 21b | Seq. ID No. 31 | 9485 | 9497 | 9793 | Seq. ID No. 32 |
| 23a | Seq. ID No. 33 | 11845 | 11374 | 10595 | Seq. ID No. 34 |
| 23b | Seq. ID No. 35 | 11845 | 11830 | 10595 | Seq. ID No. 36 |
| 24 | Seq. ID No. 37 | 12908 | 12905 | 11823 | Seq. ID No. 38 |
| 32 | Seq. ID No. 39 | 15875 | 15875 | 15789 | Seq. ID No. 40 |
| 36 | Seq. ID No. 41 | 19431 | 19386 | 18634 | Seq. ID No. 42 |
| 37 | Seq. ID No. 43 | 19372 | 19429 | 20076 | Seq. ID No. 44 |
| 38a | Seq. ID No. 45 | 21796 | 21769 | 20186 | Seq. ID No. 46 |
| 38b | Seq. ID No. 47 | 21796 | 21772 | 20186 | Seq. ID No. 48 |
| 39a | Seq. ID No. 49 | 22265 | 22238 | 21786 | Seq. ID No. 50 |
| 39b | Seq. ID No. 51 | 22265 | 22262 | 21786 | Seq. ID No. 52 |
| 40 | Seq. ID No. 53 | 23449 | 23437 | 22238 | Seq. ID No. 54 |
| 41a | Seq. ID No. 55 | 24645 | 24183 | 23434 | Seq. ID No. 56 |
| 41b | Seq. ID No. 57 | 24645 | 24402 | 23434 | Seq. ID No. 58 |
| 41c | Seq. ID No. 59 | 24645 | 24630 | 23434 | Seq. ID No. 60 |
| 43b | Seq. ID No. 61 | 25392 | 25353 | 24997 | Seq. ID No. 62 |
| 43c | Seq. ID No. 63 | 25392 | 25359 | 24997 | Seq. ID No. 64 |
| 44a | Seq. ID No. 65 | 25001 | 25028 | 25330 | Seq. ID No. 66 |
| 44b | Seq. ID No. 67 | 25001 | 25016 | 25330 | Seq. ID No. 68 |
| 45 | Seq. ID No. 69 | 25702 | 25654 | 25403 | Seq. ID No. 70 |
| 46a | Seq. ID No. 71 | 26225 | 26141 | 25800 | Seq. ID No. 72 |
| 46b | Seq. ID No. 73 | 26225 | 26153 | 25800 | Seq. ID No. 74 |
| 47 | Seq. ID No. 75 | 27661 | 27643 | 26195 | Seq. ID No. 76 |
| 48a | Seq. ID No. 77 | 29105 | 28781 | 27696 | Seq. ID No. 78 |
| 48b | Seq. ID No. 79 | 29105 | 28973 | 27696 | Seq. ID No. 80 |
| 48c | Seq. ID No. 81 | 29105 | 29090 | 27696 | Seq. ID No. 82 |
| 50 | Seq. ID No. 83 | 30169 | 30154 | 29102 | Seq. ID No. 84 |
| 51a | Seq. ID No. 85 | 31759 | 31072 | 30353 | Seq. ID No. 86 |
| 51b | Seq. ID No. 87 | 31759 | 31693 | 30353 | Seq. ID No. 88 |
| 51c | Seq. ID No. 89 | 31759 | 31741 | 30353 | Seq. ID No. 90 |
| 55 | Seq. ID No. 91 | 32149 | 32128 | 31895 | Seq. ID No. 92 |
| 56b | Seq. ID No. 93 | 32234 | 32231 | 31968 | Seq. ID No. 94 |
| 57a | Seq. ID No. 95 | 33738 | 33723 | 32455 | Seq. ID No. 96 |
| 57b | Seq. ID No. 97 | 33738 | 33732 | 32455 | Seq. ID No. 98 |
| 58 | Seq. ID No. 99 | 32611 | 32644 | 32934 | Seq. ID No. 100 |
| 60a | Seq. ID No. 101 | 34825 | 34786 | 33947 | Seq. ID No. 102 |
| 60b | Seq. ID No. 103 | 34825 | 34804 | 33947 | Seq. ID No. 104 |
| 61 | Seq. ID No. 105 | 34344 | 34344 | 34751 | Seq. ID No. 106 |
| 62a | Seq. ID No. 107 | 36496 | 36334 | 34877 | Seq. ID No. 108 |
| 62b | Seq. ID No. 109 | 36496 | 36415 | 34877 | Seq. ID No. 110 |
| 62c | Seq. ID No. 111 | 36496 | 36484 | 34877 | Seq. ID No. 112 |
| 63a | Seq. ID No. 113 | 35069 | 35102 | 35563 | Seq. ID No. 114 |
| 63b | Seq. ID No. 115 | 35069 | 35072 | 35563 | Seq. ID No. 116 |
| 65a | Seq. ID No. 117 | 37245 | 37158 | 36634 | Seq. ID No. 118 |
| 65b | Seq. ID No. 119 | 37245 | 37203 | 36634 | Seq. ID No. 120 |
| 65c | Seq. ID No. 121 | 37245 | 37242 | 36634 | Seq. ID No. 122 |
| 66a | Seq. ID No. 123 | 36563 | 36749 | 37030 | Seq. ID No. 124 |
| 67 | Seq. ID No. 125 | 37706 | 37694 | 37254 | Seq. ID No. 126 |
| 69 | Seq. ID No. 127 | 38141 | 38078 | 37803 | Seq. ID No. 128 |
| 71 | Seq. ID No. 129 | 37829 | 37862 | 38722 | Seq. ID No. 130 |
| 73a | Seq. ID No. 131 | 39233 | 39248 | 39472 | Seq. ID No. 132 |
| 73b | Seq. ID No. 133 | 39233 | 39245 | 39472 | Seq. ID No. 134 |
| 74 | Seq. ID No. 135 | 38701 | 38719 | 40068 | Seq. ID No. 136 |
| 77 | Seq. ID No. 137 | 40223 | 40232 | 41029 | Seq. ID No. 138 |
| 79 | Seq. ID No. 139 | 41098 | 41098 | 41607 | Seq. ID No. 140 |
| 80a | Seq. ID No. 141 | 41294 | 41375 | 41902 | Seq. ID No. 142 |
| 80b | Seq. ID No. 143 | 41294 | 41354 | 41902 | Seq. ID No. 144 |
| 80c | Seq. ID No. 145 | 41294 | 41300 | 41902 | Seq. ID No. 146 |
| 81a | Seq. ID No. 147 | 42667 | 42658 | 42107 | Seq. ID No. 148 |
| 81b | Seq. ID No. 149 | 42667 | 42664 | 42107 | Seq. ID No. 150 |
| 83 | Seq. ID No. 151 | 42875 | 42875 | 43096 | Seq. ID No. 152 |
| 85a | Seq. ID No. 153 | 43769 | 43976 | 44563 | Seq. ID No. 154 |
| 85b | Seq. ID No. 155 | 43769 | 43829 | 44563 | Seq. ID No. 156 |
| 85c | Seq. ID No. 157 | 43769 | 43811 | 44563 | Seq. ID No. 158 |
| 86 | Seq. ID No. 159 | 44846 | 44825 | 44592 | Seq. ID No. 160 |
| 87a | Seq. ID No. 161 | 45417 | 45315 | 45064 | Seq. ID No. 162 |
| 87b | Seq. ID No. 163 | 45417 | 45351 | 45064 | Seq. ID No. 164 |
| 87c | Seq. ID No. 165 | 45417 | 45402 | 45064 | Seq. ID No. 166 |
| 88 | Seq. ID No. 167 | 44548 | 44560 | 45258 | Seq. ID No. 168 |
| 89 | Seq. ID No. 169 | 45592 | 45550 | 45296 | Seq. ID No. 170 |
| 91a | Seq. ID No. 171 | 45068 | 45278 | 46252 | Seq. ID No. 172 |
| 91b | Seq. ID No. 173 | 45068 | 45194 | 46252 | Seq. ID No. 174 |
| 91c | Seq. ID No. 175 | 45068 | 45107 | 46252 | Seq. ID No. 176 |
| 91d | Seq. ID No. 177 | 45068 | 45074 | 46252 | Seq. ID No. 178 |
| 93a | Seq. ID No. 179 | 46421 | 46619 | 47563 | Seq. ID No. 180 |
| 93b | Seq. ID No. 181 | 46421 | 46454 | 47563 | Seq. ID No. 182 |
| 93c | Seq. ID No. 183 | 46421 | 46436 | 47563 | Seq. ID No. 184 |
| 94 | Seq. ID No. 185 | 47630 | 47630 | 47830 | Seq. ID No. 186 |
| 95b | Seq. ID No. 187 | 48482 | 48413 | 47895 | Seq. ID No. 188 |
| 97b | Seq. ID No. 189 | 48738 | 48723 | 48421 | Seq. ID No. 190 |
| 99 | Seq. ID No. 191 | 50295 | 50280 | 48739 | Seq. ID No. 192 |
| 101 | Seq. ID No. 193 | 49736 | 49766 | 50266 | Seq. ID No. 194 |
| 102 | Seq. ID No. 195 | 51996 | 51981 | 50311 | Seq. ID No. 196 |
| 104a | Seq. ID No. 197 | 52783 | 52738 | 52130 | Seq. ID No. 198 |
| 104b | Seq. ID No. 199 | 52783 | 52753 | 52130 | Seq. ID No. 200 |
| 104c | Seq. ID No. 201 | 52783 | 52780 | 52130 | Seq. ID No. 202 |
| 105a | Seq. ID No. 203 | 53486 | 53087 | 52710 | Seq. ID No. 204 |
| 105b | Seq. ID No. 205 | 53486 | 53375 | 52710 | Seq. ID No. 206 |
| 106 | Seq. ID No. 207 | 53716 | 53695 | 53102 | Seq. ID No. 208 |
| 109a | Seq. ID No. 209 | 54828 | 54606 | 53938 | Seq. ID No. 210 |
| 109b | Seq. ID No. 211 | 54828 | 54780 | 53938 | Seq. ID No. 212 |
| 109c | Seq. ID No. 213 | 54828 | 54807 | 53938 | Seq. ID No. 214 |
| 109d | Seq. ID No. 215 | 54828 | 54825 | 53938 | Seq. ID No. 216 |
| 111a | Seq. ID No. 217 | 55748 | 55580 | 54822 | Seq. ID No. 218 |
| 111b | Seq. ID No. 219 | 55748 | 55748 | 54822 | Seq. ID No. 220 |
| 112a | Seq. ID No. 221 | 56518 | 56494 | 55745 | Seq. ID No. 222 |
| 112b | Seq. ID No. 223 | 56518 | 56503 | 55745 | Seq. ID No. 224 |
| 114 | Seq. ID No. 225 | 57489 | 57459 | 56506 | Seq. ID No. 226 |
| 115 | Seq. ID No. 227 | 57509 | 57536 | 58264 | Seq. ID No. 228 |
| 119a | Seq. ID No. 229 | 58330 | 58573 | 59388 | Seq. ID No. 230 |
| 119b | Seq. ID No. 231 | 58330 | 58354 | 59388 | Seq. ID No. 232 |
| 121 | Seq. ID No. 233 | 59528 | 59540 | 60505 | Seq. ID No. 234 |
| 124a | Seq. ID No. 235 | 60496 | 61084 | 62028 | Seq. ID No. 236 |
| 124b | Seq. ID No. 237 | 60496 | 60913 | 62028 | Seq. ID No. 238 |
| 124c | Seq. ID No. 239 | 60496 | 60502 | 62028 | Seq. ID No. 240 |
| 125 | Seq. ID No. 241 | 62724 | 62700 | 62170 | Seq. ID No. 242 |
| 127a | Seq. ID No. 243 | 63580 | 63556 | 62900 | Seq. ID No. 244 |
| 127b | Seq. ID No. 245 | 63580 | 63559 | 62900 | Seq. ID No. 246 |
| 127c | Seq. ID No. 247 | 63580 | 63565 | 62900 | Seq. ID No. 248 |
| 129a | Seq. ID No. 249 | 65731 | 64381 | 63731 | Seq. ID No. 250 |
| 129b | Seq. ID No. 251 | 65731 | 64777 | 63731 | Seq. ID No. 252 |
| 129c | Seq. ID No. 253 | 65731 | 65728 | 63731 | Seq. ID No. 254 |
| 132a | Seq. ID No. 255 | 64857 | 64902 | 65162 | Seq. ID No. 256 |
| 132b | Seq. ID No. 257 | 64857 | 64875 | 65162 | Seq. ID No. 258 |
| 132c | Seq. ID No. 259 | 64857 | 64860 | 65162 | Seq. ID No. 260 |
| 134 | Seq. ID No. 261 | 66670 | 66667 | 65753 | Seq. ID No. 262 |
| 135a | Seq. ID No. 263 | 67444 | 67423 | 66671 | Seq. ID No. 264 |
| 135b | Seq. ID No. 265 | 67444 | 67444 | 66671 | Seq. ID No. 266 |
| 137a | Seq. ID No. 267 | 68103 | 68073 | 67597 | Seq. ID No. 268 |
| 137b | Seq. ID No. 269 | 68103 | 68100 | 67597 | Seq. ID No. 270 |
| 138a | Seq. ID No. 271 | 70062 | 69996 | 68104 | Seq. ID No. 272 |
| 138b | Seq. ID No. 273 | 70062 | 70032 | 68104 | Seq. ID No. 274 |
| 142 | Seq. ID No. 275 | 70135 | 70213 | 70539 | Seq. ID No. 276 |
| 143 | Seq. ID No. 277 | 71697 | 71697 | 70765 | Seq. ID No. 278 |
| 144a | Seq. ID No. 279 | 72593 | 72506 | 71721 | Seq. ID No. 280 |
| 144b | Seq. ID No. 281 | 72593 | 72545 | 71721 | Seq. ID No. 282 |
| 144c | Seq. ID No. 283 | 72593 | 72563 | 71721 | Seq. ID No. 284 |
| 145a | Seq. ID No. 285 | 74289 | 72996 | 72706 | Seq. ID No. 286 |
| 145b | Seq. ID No. 287 | 74289 | 73896 | 72706 | Seq. ID No. 288 |
| 145c | Seq. ID No. 289 | 74289 | 74157 | 72706 | Seq. ID No. 290 |
| 145d | Seq. ID No. 291 | 74289 | 74226 | 72706 | Seq. ID No. 292 |
| 147 | Seq. ID No. 293 | 74501 | 74504 | 75694 | Seq. ID No. 294 |
| 151a | Seq. ID No. 295 | 77145 | 77184 | 78218 | Seq. ID No. 296 |
| 151b | Seq. ID No. 297 | 77145 | 77163 | 78218 | Seq. ID No. 298 |
| 152a | Seq. ID No. 299 | 78163 | 78220 | 78813 | Seq. ID No. 300 |
| 152b | Seq. ID No. 301 | 78163 | 78166 | 78813 | Seq. ID No. 302 |
| 153a | Seq. ID No. 303 | 78814 | 79048 | 80013 | Seq. ID No. 304 |
| 153b | Seq. ID No. 305 | 78814 | 78973 | 80013 | Seq. ID No. 306 |
| 153c | Seq. ID No. 307 | 78814 | 78835 | 80013 | Seq. ID No. 308 |
| 154 | Seq. ID No. 309 | 80014 | 80020 | 80496 | Seq. ID No. 310 |
| 155a | Seq. ID No. 311 | 82026 | 81981 | 80719 | Seq. ID No. 312 |
| 155b | Seq. ID No. 313 | 82026 | 82014 | 80719 | Seq. ID No. 314 |
| 157a | Seq. ID No. 315 | 82976 | 82856 | 82566 | Seq. ID No. 316 |
| 157b | Seq. ID No. 317 | 82976 | 82958 | 82566 | Seq. ID No. 318 |
| 158a | Seq. ID No. 319 | 83433 | 83400 | 82972 | Seq. ID No. 320 |
| 158b | Seq. ID No. 321 | 83433 | 83415 | 82972 | Seq. ID No. 322 |
| 160a | Seq. ID No. 323 | 84411 | 84378 | 83584 | Seq. ID No. 324 |
| 160b | Seq. ID No. 325 | 84411 | 84408 | 83584 | Seq. ID No. 326 |
| 162a | Seq. ID No. 327 | 85333 | 84958 | 84479 | Seq. ID No. 328 |
| 162b | Seq. ID No. 329 | 85333 | 85117 | 84479 | Seq. ID No. 330 |
| 162c | Seq. ID No. 331 | 85333 | 85276 | 84479 | Seq. ID No. 332 |
| 162d | Seq. ID No. 333 | 85333 | 85285 | 84479 | Seq. ID No. 334 |
| 162e | Seq. ID No. 335 | 85333 | 85309 | 84479 | Seq. ID No. 336 |
| 163 | Seq. ID No. 337 | 84597 | 84609 | 85145 | Seq. ID No. 338 |
| 164 | Seq. ID No. 339 | 84797 | 84812 | 85264 | Seq. ID No. 340 |
| 165a | Seq. ID No. 341 | 86252 | 86144 | 85266 | Seq. ID No. 342 |
| 165b | Seq. ID No. 343 | 86252 | 86150 | 85266 | Seq. ID No. 344 |
| 167 | Seq. ID No. 345 | 86974 | 86959 | 86120 | Seq. ID No. 346 |
| 169 | Seq. ID No. 347 | 86178 | 86193 | 86720 | Seq. ID No. 348 |
| 170 | Seq. ID No. 349 | 86757 | 86790 | 87047 | Seq. ID No. 350 |
| 171a | Seq. ID No. 351 | 87777 | 87717 | 87316 | Seq. ID No. 352 |
| 171b | Seq. ID No. 353 | 87777 | 87762 | 87316 | Seq. ID No. 354 |
| 172 | Seq. ID No. 355 | 88726 | 88726 | 87785 | Seq. ID No. 356 |
| 173 | Seq. ID No. 357 | 87518 | 87560 | 87805 | Seq. ID No. 358 |
| 174 | Seq. ID No. 359 | 87942 | 87969 | 88196 | Seq. ID No. 360 |
| 175 | Seq. ID No. 361 | 89206 | 89194 | 88793 | Seq. ID No. 362 |
| 176 | Seq. ID No. 363 | 89592 | 89589 | 89224 | Seq. ID No. 364 |
| 177a | Seq. ID No. 365 | 90825 | 90801 | 90136 | Seq. ID No. 366 |
| 177b | Seq. ID No. 367 | 90825 | 90816 | 90136 | Seq. ID No. 368 |
| 179a | Seq. ID No. 369 | 90536 | 90599 | 90823 | Seq. ID No. 370 |
| 179b | Seq. ID No. 371 | 90536 | 90596 | 90823 | Seq. ID No. 372 |
| 180a | Seq. ID No. 373 | 92164 | 91072 | 90845 | Seq. ID No. 374 |
| 180b | Seq. ID No. 375 | 92164 | 92155 | 90845 | Seq. ID No. 376 |
| 183a | Seq. ID No. 377 | 91563 | 91608 | 91928 | Seq. ID No. 378 |
| 184 | Seq. ID No. 379 | 92612 | 92597 | 92160 | Seq. ID No. 380 |
| 185 | Seq. ID No. 381 | 92821 | 92821 | 92630 | Seq. ID No. 382 |
| 186 | Seq. ID No. 383 | 93331 | 93328 | 92825 | Seq. ID No. 384 |
| 187a | Seq. ID No. 385 | 92661 | 92706 | 93110 | Seq. ID No. 386 |
| 187b | Seq. ID No. 387 | 92661 | 92670 | 93110 | Seq. ID No. 388 |
| 188 | Seq. ID No. 389 | 93727 | 93718 | 93350 | Seq. ID No. 390 |
| 189b | Seq. ID No. 391 | 94349 | 94319 | 93780 | Seq. ID No. 392 |
| 191b | Seq. ID No. 393 | 93850 | 93877 | 94344 | Seq. ID No. 394 |
| 192a | Seq. ID No. 395 | 94748 | 94742 | 94350 | Seq. ID No. 396 |
| 192b | Seq. ID No. 397 | 94748 | 94748 | 94350 | Seq. ID No. 398 |
| 193 | Seq. ID No. 399 | 94912 | 94912 | 94739 | Seq. ID No. 400 |
| 194a | Seq. ID No. 401 | 94489 | 94528 | 94773 | Seq. ID No. 402 |
| 194b | Seq. ID No. 403 | 94489 | 94516 | 94773 | Seq. ID No. 404 |
| 195 | Seq. ID No. 405 | 95581 | 95527 | 94988 | Seq. ID No. 406 |
| 196 | Seq. ID No. 407 | 94854 | 94875 | 95147 | Seq. ID No. 408 |
| 197a | Seq. ID No. 409 | 95874 | 95862 | 95560 | Seq. ID No. 410 |
| 197b | Seq. ID No. 411 | 95874 | 95871 | 95560 | Seq. ID No. 412 |
| 198a | Seq. ID No. 413 | 96273 | 96237 | 95890 | Seq. ID No. 414 |
| 198b | Seq. ID No. 415 | 96273 | 96258 | 95890 | Seq. ID No. 416 |
| 200 | Seq. ID No. 417 | 96560 | 96557 | 96294 | Seq. ID No. 418 |
| 201a | Seq. ID No. 419 | 96178 | 96340 | 96564 | Seq. ID No. 420 |
| 202 | Seq. ID No. 421 | 96786 | 96786 | 96592 | Seq. ID No. 422 |
| 203 | Seq. ID No. 423 | 97210 | 97207 | 96776 | Seq. ID No. 424 |
| 204 | Seq. ID No. 425 | 97870 | 97867 | 97211 | Seq. ID No. 426 |
| 205 | Seq. ID No. 427 | 98245 | 98227 | 97871 | Seq. ID No. 428 |
| 206 | Seq. ID No. 429 | 98536 | 98524 | 98249 | Seq. ID No. 430 |
| 207 | Seq. ID No. 431 | 98031 | 98031 | 98282 | Seq. ID No. 432 |
| 208a | Seq. ID No. 433 | 99436 | 99367 | 98567 | Seq. ID No. 434 |
| 208b | Seq. ID No. 435 | 99436 | 99412 | 98567 | Seq. ID No. 436 |
| 210 | Seq. ID No. 437 | 99792 | 99738 | 99445 | Seq. ID No. 438 |
| 211 | Seq. ID No. 439 | 100364 | 100361 | 99738 | Seq. ID No. 440 |
| 213 | Seq. ID No. 441 | 101050 | 101020 | 100388 | Seq. ID No. 442 |
| 215a | Seq. ID No. 443 | 101510 | 101444 | 101058 | Seq. ID No. 444 |
| 218 | Seq. ID No. 445 | 102897 | 102885 | 102442 | Seq. ID No. 446 |
| 219 | Seq. ID No. 447 | 104259 | 104244 | 103060 | Seq. ID No. 448 |
| 220a | Seq. ID No. 449 | 106476 | 106326 | 104386 | Seq. ID No. 450 |
| 220b | Seq. ID No. 451 | 106476 | 106428 | 104386 | Seq. ID No. 452 |
| 220c | Seq. ID No. 453 | 106476 | 106476 | 104386 | Seq. ID No. 454 |
| 223a | Seq. ID No. 455 | 105032 | 105092 | 105457 | Seq. ID No. 456 |
| 223b | Seq. ID No. 457 | 105032 | 105065 | 105457 | Seq. ID No. 458 |
| 226a | Seq. ID No. 459 | 107077 | 106960 | 106580 | Seq. ID No. 460 |
| 226b | Seq. ID No. 461 | 107077 | 107050 | 106580 | Seq. ID No. 462 |
| 227 | Seq. ID No. 463 | 107524 | 107506 | 107096 | Seq. ID No. 464 |
| 228a | Seq. ID No. 465 | 111563 | 111203 | 107883 | Seq. ID No. 466 |
| 228b | Seq. ID No. 467 | 111563 | 111269 | 107883 | Seq. ID No. 468 |
| 228c | Seq. ID No. 469 | 111563 | 111461 | 107883 | Seq. ID No. 470 |
| 228d | Seq. ID No. 471 | 111563 | 111542 | 107883 | Seq. ID No. 472 |
| 232a | Seq. ID No. 473 | 108687 | 108723 | 109223 | Seq. ID No. 474 |
| 232b | Seq. ID No. 475 | 108687 | 108690 | 109223 | Seq. ID No. 476 |
| 235a | Seq. ID No. 477 | 110593 | 110707 | 111144 | Seq. ID No. 478 |
| 235b | Seq. ID No. 479 | 110593 | 110626 | 111144 | Seq. ID No. 480 |
| 236a | Seq. ID No. 481 | 115224 | 115071 | 111643 | Seq. ID No. 482 |
| 236b | Seq. ID No. 483 | 115224 | 115212 | 111643 | Seq. ID No. 484 |
| 236c | Seq. ID No. 485 | 115224 | 115221 | 111643 | Seq. ID No. 486 |
| 239a | Seq. ID No. 487 | 111971 | 112139 | 112507 | Seq. ID No. 488 |
| 239b | Seq. ID No. 489 | 111971 | 112097 | 112507 | Seq. ID No. 490 |
| 239c | Seq. ID No. 491 | 111971 | 112085 | 112507 | Seq. ID No. 492 |
| 240 | Seq. ID No. 493 | 112508 | 112601 | 112867 | Seq. ID No. 494 |
| 241 | Seq. ID No. 495 | 113123 | 113129 | 113374 | Seq. ID No. 496 |
| 245a | Seq. ID No. 497 | 116934 | 116292 | 115537 | Seq. ID No. 498 |
| 245b | Seq. ID No. 499 | 116934 | 116775 | 115537 | Seq. ID No. 500 |
| 245c | Seq. ID No. 501 | 116934 | 116895 | 115537 | Seq. ID No. 502 |
| 246a | Seq. ID No. 503 | 118257 | 118056 | 117070 | Seq. ID No. 504 |
| 246b | Seq. ID No. 505 | 118257 | 118245 | 117070 | Seq. ID No. 506 |
| 247 | Seq. ID No. 507 | 117578 | 117584 | 118102 | Seq. ID No. 508 |
| 248a | Seq. ID No. 509 | 119490 | 119358 | 118264 | Seq. ID No. 510 |
| 248b | Seq. ID No. 511 | 119490 | 119478 | 118264 | Seq. ID No. 512 |
| 249a | Seq. ID No. 513 | 121252 | 121225 | 119774 | Seq. ID No. 514 |
| 249b | Seq. ID No. 515 | 121252 | 121252 | 119774 | Seq. ID No. 516 |
| 251 | Seq. ID No. 517 | 122162 | 122150 | 121326 | Seq. ID No. 518 |
| 253a | Seq. ID No. 519 | 122435 | 122387 | 122163 | Seq. ID No. 520 |
| 253b | Seq. ID No. 521 | 122435 | 122432 | 122163 | Seq. ID No. 522 |
| 254 | Seq. ID No. 523 | 123069 | 123045 | 122416 | Seq. ID No. 524 |
| 255a | Seq. ID No. 525 | 124654 | 123817 | 123116 | Seq. ID No. 526 |
| 255b | Seq. ID No. 527 | 124654 | 124408 | 123116 | Seq. ID No. 528 |
| 255c | Seq. ID No. 529 | 124654 | 124639 | 123116 | Seq. ID No. 530 |
| 255d | Seq. ID No. 531 | 124654 | 124645 | 123116 | Seq. ID No. 532 |
| 258 | Seq. ID No. 533 | 125557 | 125539 | 124658 | Seq. ID No. 534 |
| 259a | Seq. ID No. 535 | 126322 | 126256 | 125558 | Seq. ID No. 536 |
| 259b | Seq. ID No. 537 | 126322 | 126292 | 125558 | Seq. ID No. 538 |
| 259c | Seq. ID No. 539 | 126322 | 126298 | 125558 | Seq. ID No. 540 |
| 260 | Seq. ID No. 541 | 126904 | 126901 | 126533 | Seq. ID No. 542 |
| 262 | Seq. ID No. 543 | 127474 | 127474 | 126965 | Seq. ID No. 544 |
| 263a | Seq. ID No. 545 | 126519 | 126645 | 126968 | Seq. ID No. 546 |
| 263b | Seq. ID No. 547 | 126519 | 126603 | 126968 | Seq. ID No. 548 |
| 264b | Seq. ID No. 549 | 126969 | 127005 | 127382 | Seq. ID No. 550 |
| 265 | Seq. ID No. 551 | 128443 | 128389 | 127691 | Seq. ID No. 552 |
| 267b | Seq. ID No. 553 | 129002 | 128912 | 128487 | Seq. ID No. 554 |
| 267c | Seq. ID No. 555 | 129002 | 128996 | 128487 | Seq. ID No. 556 |
| 268 | Seq. ID No. 557 | 128485 | 128500 | 128733 | Seq. ID No. 558 |
| 269a | Seq. ID No. 559 | 129717 | 129609 | 129055 | Seq. ID No. 560 |
| 269b | Seq. ID No. 561 | 129717 | 129708 | 129055 | Seq. ID No. 562 |
| 271a | Seq. ID No. 563 | 131163 | 131118 | 129856 | Seq. ID No. 564 |
| 271b | Seq. ID No. 565 | 131163 | 131151 | 129856 | Seq. ID No. 566 |
| 273a | Seq. ID No. 567 | 133346 | 132791 | 131694 | Seq. ID No. 568 |
| 273b | Seq. ID No. 569 | 133346 | 133343 | 131694 | Seq. ID No. 570 |
| 277a | Seq. ID No. 571 | 135131 | 134891 | 134046 | Seq. ID No. 572 |
| 277b | Seq. ID No. 573 | 135131 | 135077 | 134046 | Seq. ID No. 574 |
| 277c | Seq. ID No. 575 | 135131 | 135119 | 134046 | Seq. ID No. 576 |
| 279a | Seq. ID No. 577 | 135434 | 135626 | 135994 | Seq. ID No. 578 |
| 279b | Seq. ID No. 579 | 135434 | 135437 | 135994 | Seq. ID No. 580 |
| 280a | Seq. ID No. 581 | 138094 | 137200 | 136100 | Seq. ID No. 582 |
| 280b | Seq. ID No. 583 | 138094 | 137593 | 136100 | Seq. ID No. 584 |
| 280c | Seq. ID No. 585 | 138094 | 137926 | 136100 | Seq. ID No. 586 |
| 280d | Seq. ID No. 587 | 138094 | 138052 | 136100 | Seq. ID No. 588 |
| 282 | Seq. ID No. 589 | 139430 | 139388 | 139116 | Seq. ID No. 590 |
| 283a | Seq. ID No. 591 | 138704 | 138719 | 139567 | Seq. ID No. 592 |
| 283b | Seq. ID No. 593 | 138704 | 138710 | 139567 | Seq. ID No. 594 |
| 284a | Seq. ID No. 595 | 139568 | 139592 | 140257 | Seq. ID No. 596 |
| 284b | Seq. ID No. 597 | 139568 | 139574 | 140257 | Seq. ID No. 598 |
| 285b | Seq. ID No. 599 | 141025 | 141022 | 140702 | Seq. ID No. 600 |
| 286a | Seq. ID No. 601 | 140271 | 140286 | 141035 | Seq. ID No. 602 |
| 286b | Seq. ID No. 603 | 140271 | 140277 | 141035 | Seq. ID No. 604 |
| 287 | Seq. ID No. 605 | 142180 | 142153 | 141284 | Seq. ID No. 606 |
| 290a | Seq. ID No. 607 | 142653 | 142590 | 142177 | Seq. ID No. 608 |
| 290b | Seq. ID No. 609 | 142653 | 142638 | 142177 | Seq. ID No. 610 |
| 293 | Seq. ID No. 611 | 142822 | 142855 | 143292 | Seq. ID No. 612 |
| 294 | Seq. ID No. 613 | 143980 | 143974 | 143411 | Seq. ID No. 614 |
| 296 | Seq. ID No. 615 | 146154 | 146151 | 143974 | Seq. ID No. 616 |
| 297 | Seq. ID No. 617 | 144242 | 144227 | 143988 | Seq. ID No. 618 |
| 300b | Seq. ID No. 619 | 145417 | 145420 | 145899 | Seq. ID No. 620 |
| 301a | Seq. ID No. 621 | 147294 | 147237 | 146278 | Seq. ID No. 622 |
| 301b | Seq. ID No. 623 | 147294 | 147264 | 146278 | Seq. ID No. 624 |
| 304a | Seq. ID No. 625 | 149441 | 149135 | 147252 | Seq. ID No. 626 |
| 304b | Seq. ID No. 627 | 149441 | 149423 | 147252 | Seq. ID No. 628 |
| 307 | Seq. ID No. 629 | 149829 | 149781 | 149401 | Seq. ID No. 630 |
| 308 | Seq. ID No. 631 | 150040 | 150016 | 149783 | Seq. ID No. 632 |
| 309 | Seq. ID No. 633 | 149973 | 149973 | 150149 | Seq. ID No. 634 |
| 310 | Seq. ID No. 635 | 151326 | 151308 | 150298 | Seq. ID No. 636 |
| 311 | Seq. ID No. 637 | 153222 | 153168 | 151447 | Seq. ID No. 638 |
| 313a | Seq. ID No. 639 | 153970 | 153457 | 153188 | Seq. ID No. 640 |
| 313b | Seq. ID No. 641 | 153970 | 153496 | 153188 | Seq. ID No. 642 |
| 313c | Seq. ID No. 643 | 153970 | 153952 | 153188 | Seq. ID No. 644 |
| 313d | Seq. ID No. 645 | 153970 | 153955 | 153188 | Seq. ID No. 646 |
| 313e | Seq. ID No. 647 | 153970 | 153961 | 153188 | Seq. ID No. 648 |
| 315 | Seq. ID No. 649 | 154835 | 154826 | 153939 | Seq. ID No. 650 |
| 316a | Seq. ID No. 651 | 155263 | 155209 | 154826 | Seq. ID No. 652 |
| 317a | Seq. ID No. 653 | 155877 | 155646 | 155287 | Seq. ID No. 654 |
| 317b | Seq. ID No. 655 | 155877 | 155865 | 155287 | Seq. ID No. 656 |
| 318a | Seq. ID No. 657 | 157499 | 157280 | 156426 | Seq. ID No. 658 |
| 318b | Seq. ID No. 659 | 157499 | 157304 | 156426 | Seq. ID No. 660 |
| 318c | Seq. ID No. 661 | 157499 | 157367 | 156426 | Seq. ID No. 662 |
| 318d | Seq. ID No. 663 | 157499 | 157451 | 156426 | Seq. ID No. 664 |
| 318e | Seq. ID No. 665 | 157499 | 157460 | 156426 | Seq. ID No. 666 |
| 320 | Seq. ID No. 667 | 156670 | 156700 | 156975 | Seq. ID No. 668 |
| 321 | Seq. ID No. 669 | 158142 | 158139 | 157312 | Seq. ID No. 670 |
| 323a | Seq. ID No. 671 | 158413 | 158341 | 158129 | Seq. ID No. 672 |
| 323b | Seq. ID No. 673 | 158413 | 158395 | 158129 | Seq. ID No. 674 |
| 324 | Seq. ID No. 675 | 159203 | 159200 | 158373 | Seq. ID No. 676 |
| 327a | Seq. ID No. 677 | 159964 | 159682 | 159233 | Seq. ID No. 678 |
| 327b | Seq. ID No. 679 | 159964 | 159844 | 159233 | Seq. ID No. 680 |
| 327c | Seq. ID No. 681 | 159964 | 159937 | 159233 | Seq. ID No. 682 |
| 329 | Seq. ID No. 683 | 161034 | 161034 | 159934 | Seq. ID No. 684 |
| 330a | Seq. ID No. 685 | 161885 | 162272 | 163120 | Seq. ID No. 686 |
| 330b | Seq. ID No. 687 | 161885 | 161975 | 163120 | Seq. ID No. 688 |
| 330c | Seq. ID No. 689 | 161885 | 161888 | 163120 | Seq. ID No. 690 |
| 331 | Seq. ID No. 691 | 162945 | 162951 | 163187 | Seq. ID No. 692 |
| 332 | Seq. ID No. 693 | 163441 | 163441 | 163238 | Seq. ID No. 694 |
| 333 | Seq. ID No. 695 | 163242 | 163248 | 163478 | Seq. ID No. 696 |
| 334 | Seq. ID No. 697 | 163339 | 163339 | 163563 | Seq. ID No. 698 |
| 335a | Seq. ID No. 699 | 163139 | 163193 | 163786 | Seq. ID No. 700 |
| 335b | Seq. ID No. 701 | 163139 | 163178 | 163786 | Seq. ID No. 702 |
| 337 | Seq. ID No. 703 | 163774 | 163798 | 164847 | Seq. ID No. 704 |
| 338a | Seq. ID No. 705 | 164835 | 164961 | 165473 | Seq. ID No. 706 |
| 338b | Seq. ID No. 707 | 164835 | 164952 | 165473 | Seq. ID No. 708 |
| 338c | Seq. ID No. 709 | 164835 | 164889 | 165473 | Seq. ID No. 710 |
| 341a | Seq. ID No. 711 | 165550 | 165709 | 166422 | Seq. ID No. 712 |
| 341b | Seq. ID No. 713 | 165550 | 165550 | 166422 | Seq. ID No. 714 |
| 342a | Seq. ID No. 715 | 167866 | 166741 | 166517 | Seq. ID No. 716 |
| 342b | Seq. ID No. 717 | 167866 | 166810 | 166517 | Seq. ID No. 718 |
| 342c | Seq. ID No. 719 | 167866 | 166951 | 166517 | Seq. ID No. 720 |
| 342d | Seq. ID No. 721 | 167866 | 167164 | 166517 | Seq. ID No. 722 |
| 342e | Seq. ID No. 723 | 167866 | 167602 | 166517 | Seq. ID No. 724 |
| 342f | Seq. ID No. 725 | 167866 | 167791 | 166517 | Seq. ID No. 726 |
| 342g | Seq. ID No. 727 | 167866 | 167824 | 166517 | Seq. ID No. 728 |
| 342h | Seq. ID No. 729 | 167866 | 167857 | 166517 | Seq. ID No. 730 |
| 344a | Seq. ID No. 731 | 167906 | 167942 | 168580 | Seq. ID No. 732 |
| 344b | Seq. ID No. 733 | 167906 | 167918 | 168580 | Seq. ID No. 734 |
| 345 | Seq. ID No. 735 | 169267 | 169258 | 168590 | Seq. ID No. 736 |
| 346a | Seq. ID No. 737 | 169209 | 169296 | 169616 | Seq. ID No. 738 |
| 346b | Seq. ID No. 739 | 169209 | 169212 | 169616 | Seq. ID No. 740 |
| 347 | Seq. ID No. 741 | 170242 | 170227 | 169742 | Seq. ID No. 742 |
| 349a | Seq. ID No. 743 | 170547 | 170637 | 170948 | Seq. ID No. 744 |
| 349b | Seq. ID No. 745 | 170547 | 170559 | 170948 | Seq. ID No. 746 |
| 349c | Seq. ID No. 747 | 170547 | 170550 | 170948 | Seq. ID No. 748 |
| 350a | Seq. ID No. 749 | 171289 | 171334 | 172113 | Seq. ID No. 750 |
| 350b | Seq. ID No. 751 | 171289 | 171304 | 172113 | Seq. ID No. 752 |
| 351 | Seq. ID No. 753 | 172083 | 172116 | 172757 | Seq. ID No. 754 |
| 352a | Seq. ID No. 755 | 172736 | 173237 | 173545 | Seq. ID No. 756 |
| 352b | Seq. ID No. 757 | 172736 | 173222 | 173545 | Seq. ID No. 758 |
| 352c | Seq. ID No. 759 | 172736 | 172754 | 173545 | Seq. ID No. 760 |
| 354a | Seq. ID No. 761 | 173511 | 173913 | 174341 | Seq. ID No. 762 |
| 354b | Seq. ID No. 763 | 173511 | 173565 | 174341 | Seq. ID No. 764 |
| 354c | Seq. ID No. 765 | 173511 | 173535 | 174341 | Seq. ID No. 766 |
| 355 | Seq. ID No. 767 | 174702 | 174594 | 174361 | Seq. ID No. 768 |
| 356a | Seq. ID No. 769 | 174424 | 174847 | 175176 | Seq. ID No. 770 |
| 356b | Seq. ID No. 771 | 174424 | 174610 | 175176 | Seq. ID No. 772 |
| 356c | Seq. ID No. 773 | 174424 | 174538 | 175176 | Seq. ID No. 774 |
| 356d | Seq. ID No. 775 | 174424 | 174469 | 175176 | Seq. ID No. 776 |
| 358a | Seq. ID No. 777 | 175629 | 176586 | 176951 | Seq. ID No. 778 |
| 358b | Seq. ID No. 779 | 175629 | 175692 | 176951 | Seq. ID No. 780 |
| 358c | Seq. ID No. 781 | 175629 | 175653 | 176951 | Seq. ID No. 782 |
| 362 | Seq. ID No. 783 | 177179 | 177206 | 178258 | Seq. ID No. 784 |
| 365a | Seq. ID No. 785 | 178795 | 178840 | 179430 | Seq. ID No. 786 |
| 365b | Seq. ID No. 787 | 178795 | 178807 | 179430 | Seq. ID No. 788 |
| 366b | Seq. ID No. 789 | 179373 | 179436 | 180101 | Seq. ID No. 790 |
| 366c | Seq. ID No. 791 | 179373 | 179427 | 180101 | Seq. ID No. 792 |
| 367a | Seq. ID No. 793 | 181005 | 180984 | 180184 | Seq. ID No. 794 |
| 367b | Seq. ID No. 795 | 181005 | 181002 | 180184 | Seq. ID No. 796 |
| 371a | Seq. ID No. 797 | 181201 | 181216 | 181662 | Seq. ID No. 798 |
| 371b | Seq. ID No. 799 | 181201 | 181204 | 181662 | Seq. ID No. 800 |
| 372a | Seq. ID No. 801 | 184610 | 183770 | 181809 | Seq. ID No. 802 |
| 372b | Seq. ID No. 803 | 184610 | 184463 | 181809 | Seq. ID No. 804 |
| 372c | Seq. ID No. 805 | 184610 | 184562 | 181809 | Seq. ID No. 806 |
| 372d | Seq. ID No. 807 | 184610 | 184598 | 181809 | Seq. ID No. 808 |
| 374 | Seq. ID No. 809 | 182287 | 182368 | 182628 | Seq. ID No. 810 |
| 377a | Seq. ID No. 811 | 183658 | 183769 | 184497 | Seq. ID No. 812 |
| 377b | Seq. ID No. 813 | 183658 | 183682 | 184497 | Seq. ID No. 814 |
| 378a | Seq. ID No. 815 | 185701 | 185635 | 184616 | Seq. ID No. 816 |
| 378b | Seq. ID No. 817 | 185701 | 185677 | 184616 | Seq. ID No. 818 |
| 382b | Seq. ID No. 819 | 186504 | 186492 | 185782 | Seq. ID No. 820 |
| 383a | Seq. ID No. 821 | 188140 | 188092 | 187067 | Seq. ID No. 822 |
| 383b | Seq. ID No. 823 | 188140 | 188104 | 187067 | Seq. ID No. 824 |
| 384a | Seq. ID No. 825 | 189536 | 188333 | 188085 | Seq. ID No. 826 |
| 384b | Seq. ID No. 827 | 189536 | 189509 | 188085 | Seq. ID No. 828 |
| 385a | Seq. ID No. 829 | 190440 | 190362 | 189526 | Seq. ID No. 830 |
| 385b | Seq. ID No. 831 | 190440 | 190434 | 189526 | Seq. ID No. 832 |
| 386 | Seq. ID No. 833 | 190535 | 190535 | 190431 | Seq. ID No. 834 |
| 387 | Seq. ID No. 835 | 192036 | 192021 | 190792 | Seq. ID No. 836 |
| 390 | Seq. ID No. 837 | 192805 | 192781 | 192056 | Seq. ID No. 838 |
| 391 | Seq. ID No. 839 | 193828 | 193816 | 192968 | Seq. ID No. 840 |
| 393b | Seq. ID No. 841 | 193199 | 193205 | 193543 | Seq. ID No. 842 |
| 395a | Seq. ID No. 843 | 194052 | 194199 | 194792 | Seq. ID No. 844 |
| 395b | Seq. ID No. 845 | 194052 | 194088 | 194792 | Seq. ID No. 846 |
| 395c | Seq. ID No. 847 | 194052 | 194058 | 194792 | Seq. ID No. 848 |
| 396 | Seq. ID No. 849 | 194817 | 194817 | 195026 | Seq. ID No. 850 |
| 398 | Seq. ID No. 851 | 195036 | 195093 | 195338 | Seq. ID No. 852 |
| 400a | Seq. ID No. 853 | 195530 | 195740 | 196156 | Seq. ID No. 854 |
| 400b | Seq. ID No. 855 | 195530 | 195551 | 196156 | Seq. ID No. 856 |
| 401a | Seq. ID No. 857 | 197561 | 197516 | 196254 | Seq. ID No. 858 |
| 401b | Seq. ID No. 859 | 197561 | 197549 | 196254 | Seq. ID No. 860 |
| 403a | Seq. ID No. 861 | 200060 | 199496 | 198111 | Seq. ID No. 862 |
| 403b | Seq. ID No. 863 | 200060 | 199943 | 198111 | Seq. ID No. 864 |
| 403c | Seq. ID No. 865 | 200060 | 200000 | 198111 | Seq. ID No. 866 |
| 403d | Seq. ID No. 867 | 200060 | 200003 | 198111 | Seq. ID No. 868 |
| 406a | Seq. ID No. 869 | 201691 | 201646 | 200384 | Seq. ID No. 870 |
| 406b | Seq. ID No. 871 | 201691 | 201679 | 200384 | Seq. ID No. 872 |
| 409a | Seq. ID No. 873 | 203082 | 202980 | 202234 | Seq. ID No. 874 |
| 409b | Seq. ID No. 875 | 203082 | 203079 | 202234 | Seq. ID No. 876 |
| 410a | Seq. ID No. 877 | 205212 | 205185 | 203377 | Seq. ID No. 878 |
| 410b | Seq. ID No. 879 | 205212 | 205200 | 203377 | Seq. ID No. 880 |
| 410c | Seq. ID No. 881 | 205212 | 205209 | 203377 | Seq. ID No. 882 |
| 413a | Seq. ID No. 883 | 206260 | 205960 | 205544 | Seq. ID No. 884 |
| 413b | Seq. ID No. 885 | 206260 | 206224 | 205544 | Seq. ID No. 886 |
| 416a | Seq. ID No. 887 | 206529 | 206499 | 206266 | Seq. ID No. 888 |
| 416b | Seq. ID No. 889 | 206529 | 206529 | 206266 | Seq. ID No. 890 |
| 417 | Seq. ID No. 891 | 206866 | 206842 | 206519 | Seq. ID No. 892 |
| 418a | Seq. ID No. 893 | 208884 | 208674 | 206959 | Seq. ID No. 894 |
| 418b | Seq. ID No. 895 | 208884 | 208827 | 206959 | Seq. ID No. 896 |
| 419a | Seq. ID No. 897 | 210885 | 210405 | 209041 | Seq. ID No. 898 |
| 419b | Seq. ID No. 899 | 210885 | 210609 | 209041 | Seq. ID No. 900 |
| 419c | Seq. ID No. 901 | 210885 | 210864 | 209041 | Seq. ID No. 902 |
| 420a | Seq. ID No. 903 | 209648 | 209660 | 209893 | Seq. ID No. 904 |
| 420b | Seq. ID No. 905 | 209648 | 209657 | 209893 | Seq. ID No. 906 |
| 421 | Seq. ID No. 907 | 212336 | 212297 | 211083 | Seq. ID No. 908 |
| 422 | Seq. ID No. 909 | 211732 | 211765 | 211989 | Seq. ID No. 910 |
| 424a | Seq. ID No. 911 | 213555 | 213390 | 212911 | Seq. ID No. 912 |
| 424b | Seq. ID No. 913 | 213555 | 213522 | 212911 | Seq. ID No. 914 |
| 424c | Seq. ID No. 915 | 213555 | 213525 | 212911 | Seq. ID No. 916 |
| 426a | Seq. ID No. 917 | 213625 | 213637 | 213936 | Seq. ID No. 918 |
| 426b | Seq. ID No. 919 | 213625 | 213631 | 213936 | Seq. ID No. 920 |
| 427a | Seq. ID No. 921 | 214643 | 214553 | 214074 | Seq. ID No. 922 |
| 427b | Seq. ID No. 923 | 214643 | 214589 | 214074 | Seq. ID No. 924 |
| 427c | Seq. ID No. 925 | 214643 | 214625 | 214074 | Seq. ID No. 926 |
| 429a | Seq. ID No. 927 | 215579 | 215441 | 214998 | Seq. ID No. 928 |
| 429b | Seq. ID No. 929 | 215579 | 215498 | 214998 | Seq. ID No. 930 |
| 429c | Seq. ID No. 931 | 215579 | 215540 | 214998 | Seq. ID No. 932 |
| 429d | Seq. ID No. 933 | 215579 | 215546 | 214998 | Seq. ID No. 934 |
| 431 | Seq. ID No. 935 | 216192 | 216192 | 215455 | Seq. ID No. 936 |
| 434 | Seq. ID No. 937 | 215861 | 215876 | 216112 | Seq. ID No. 938 |
| 435 | Seq. ID No. 939 | 217111 | 217093 | 216281 | Seq. ID No. 940 |
| 437b | Seq. ID No. 941 | 216993 | 216987 | 216646 | Seq. ID No. 942 |
| 437c | Seq. ID No. 943 | 216993 | 216993 | 216646 | Seq. ID No. 944 |
| 438a | Seq. ID No. 945 | 218376 | 218307 | 217327 | Seq. ID No. 946 |
| 438b | Seq. ID No. 947 | 218376 | 218352 | 217327 | Seq. ID No. 948 |
| 438c | Seq. ID No. 949 | 218376 | 218376 | 217327 | Seq. ID No. 950 |
| 440 | Seq. ID No. 951 | 219614 | 219608 | 218307 | Seq. ID No. 952 |
| 441a | Seq. ID No. 953 | 221512 | 221416 | 219605 | Seq. ID No. 954 |
| 441b | Seq. ID No. 955 | 221512 | 221491 | 219605 | Seq. ID No. 956 |
| 443b | Seq. ID No. 957 | 220467 | 220509 | 220775 | Seq. ID No. 958 |
| 444a | Seq. ID No. 959 | 222225 | 222198 | 221506 | Seq. ID No. 960 |
| 444b | Seq. ID No. 961 | 222225 | 222213 | 221506 | Seq. ID No. 962 |
| 445a | Seq. ID No. 963 | 222816 | 222783 | 222292 | Seq. ID No. 964 |
| 445b | Seq. ID No. 965 | 222816 | 222801 | 222292 | Seq. ID No. 966 |
| 448 | Seq. ID No. 967 | 223126 | 223156 | 224475 | Seq. ID No. 968 |
| 450a | Seq. ID No. 969 | 224457 | 224481 | 225266 | Seq. ID No. 970 |
| 450b | Seq. ID No. 971 | 224457 | 224469 | 225266 | Seq. ID No. 972 |
| 451a | Seq. ID No. 973 | 226352 | 226328 | 226101 | Seq. ID No. 974 |
| 451b | Seq. ID No. 975 | 226352 | 226331 | 226101 | Seq. ID No. 976 |
| 452a | Seq. ID No. 977 | 225232 | 225328 | 226530 | Seq. ID No. 978 |
| 452b | Seq. ID No. 979 | 225232 | 225295 | 226530 | Seq. ID No. 980 |
| 452c | Seq. ID No. 981 | 225232 | 225256 | 226530 | Seq. ID No. 982 |
| 452d | Seq. ID No. 983 | 225232 | 225247 | 226530 | Seq. ID No. 984 |
| 452e | Seq. ID No. 985 | 225232 | 225232 | 226530 | Seq. ID No. 986 |
| 454b | Seq. ID No. 987 | 227703 | 227703 | 227224 | Seq. ID No. 988 |
| 455a | Seq. ID No. 989 | 226615 | 226738 | 227742 | Seq. ID No. 990 |
| 455b | Seq. ID No. 991 | 226615 | 226693 | 227742 | Seq. ID No. 992 |
| 455c | Seq. ID No. 993 | 226615 | 226627 | 227742 | Seq. ID No. 994 |
| 458a | Seq. ID No. 995 | 227816 | 227936 | 229081 | Seq. ID No. 996 |
| 458b | Seq. ID No. 997 | 227816 | 227840 | 229081 | Seq. ID No. 998 |
| 460a | Seq. ID No. 999 | 229035 | 229098 | 229883 | Seq. ID No. 1000 |
| 460b | Seq. ID No. 1001 | 229035 | 229062 | 229883 | Seq. ID No. 1002 |
| 460c | Seq. ID No. 1003 | 229035 | 229053 | 229883 | Seq. ID No. 1004 |
| 462 | Seq. ID No. 1005 | 230230 | 230239 | 230679 | Seq. ID No. 1006 |
| 463a | Seq. ID No. 1007 | 230696 | 230720 | 231208 | Seq. ID No. 1008 |
| 463b | Seq. ID No. 1009 | 230696 | 230711 | 231208 | Seq. ID No. 1010 |
| 464a | Seq. ID No. 1011 | 233073 | 231831 | 231601 | Seq. ID No. 1012 |
| 464b | Seq. ID No. 1013 | 233073 | 232560 | 231601 | Seq. ID No. 1014 |
| 464c | Seq. ID No. 1015 | 233073 | 232890 | 231601 | Seq. ID No. 1016 |
| 464d | Seq. ID No. 1017 | 233073 | 233052 | 231601 | Seq. ID No. 1018 |
| 465b | Seq. ID No. 1019 | 232439 | 232433 | 232119 | Seq. ID No. 1020 |
| 467a | Seq. ID No. 1021 | 234505 | 234430 | 233069 | Seq. ID No. 1022 |
| 467b | Seq. ID No. 1023 | 234505 | 234472 | 233069 | Seq. ID No. 1024 |
| 471a | Seq. ID No. 1025 | 235904 | 235874 | 235617 | Seq. ID No. 1026 |
| 471b | Seq. ID No. 1027 | 235904 | 235898 | 235617 | Seq. ID No. 1028 |
| 472a | Seq. ID No. 1029 | 234562 | 235432 | 235953 | Seq. ID No. 1030 |
| 472b | Seq. ID No. 1031 | 234562 | 235294 | 235953 | Seq. ID No. 1032 |
| 472c | Seq. ID No. 1033 | 234562 | 234958 | 235953 | Seq. ID No. 1034 |
| 472d | Seq. ID No. 1035 | 234562 | 234805 | 235953 | Seq. ID No. 1036 |
| 472e | Seq. ID No. 1037 | 234562 | 234796 | 235953 | Seq. ID No. 1038 |
| 472f | Seq. ID No. 1039 | 234562 | 234574 | 235953 | Seq. ID No. 1040 |
| 473 | Seq. ID No. 1041 | 235954 | 235963 | 236619 | Seq. ID No. 1042 |
| 474 | Seq. ID No. 1043 | 237869 | 237851 | 236748 | Seq. ID No. 1044 |
| 476 | Seq. ID No. 1045 | 238557 | 238551 | 238282 | Seq. ID No. 1046 |
| 477b | Seq. ID No. 1047 | 239139 | 239133 | 238819 | Seq. ID No. 1048 |
| 478a | Seq. ID No. 1049 | 238145 | 238253 | 239143 | Seq. ID No. 1050 |
| 478b | Seq. ID No. 1051 | 238145 | 238208 | 239143 | Seq. ID No. 1052 |
| 479 | Seq. ID No. 1053 | 239300 | 239318 | 239719 | Seq. ID No. 1054 |
| 480a | Seq. ID No. 1055 | 241246 | 241159 | 239954 | Seq. ID No. 1056 |
| 480b | Seq. ID No. 1057 | 241246 | 241213 | 239954 | Seq. ID No. 1058 |
| 481a | Seq. ID No. 1059 | 242130 | 242112 | 241498 | Seq. ID No. 1060 |
| 481b | Seq. ID No. 1061 | 242130 | 242127 | 241498 | Seq. ID No. 1062 |
| 482a | Seq. ID No. 1063 | 242585 | 242540 | 242109 | Seq. ID No. 1064 |
| 482b | Seq. ID No. 1065 | 242585 | 242576 | 242109 | Seq. ID No. 1066 |
| 483 | Seq. ID No. 1067 | 243003 | 243000 | 242668 | Seq. ID No. 1068 |
| 490 | Seq. ID No. 1069 | 243146 | 243158 | 244378 | Seq. ID No. 1070 |
| 491a | Seq. ID No. 1071 | 244796 | 244763 | 244506 | Seq. ID No. 1072 |
| 491b | Seq. ID No. 1073 | 244796 | 244781 | 244506 | Seq. ID No. 1074 |
| 492 | Seq. ID No. 1075 | 245550 | 245511 | 244786 | Seq. ID No. 1076 |
| 494a | Seq. ID No. 1077 | 245120 | 245192 | 245554 | Seq. ID No. 1078 |
| 495a | Seq. ID No. 1079 | 247355 | 247226 | 245949 | Seq. ID No. 1080 |
| 495b | Seq. ID No. 1081 | 247355 | 247235 | 245949 | Seq. ID No. 1082 |
| 495c | Seq. ID No. 1083 | 247355 | 247331 | 245949 | Seq. ID No. 1084 |
| 498a | Seq. ID No. 1085 | 247969 | 247912 | 247466 | Seq. ID No. 1086 |
| 498b | Seq. ID No. 1087 | 247969 | 247939 | 247466 | Seq. ID No. 1088 |
| 499a | Seq. ID No. 1089 | 249985 | 249943 | 247970 | Seq. ID No. 1090 |
| 499b | Seq. ID No. 1091 | 249985 | 249970 | 247970 | Seq. ID No. 1092 |
| 500 | Seq. ID No. 1093 | 250457 | 250439 | 250203 | Seq. ID No. 1094 |
| 502b | Seq. ID No. 1095 | 251041 | 250951 | 250475 | Seq. ID No. 1096 |
| 502c | Seq. ID No. 1097 | 251041 | 251026 | 250475 | Seq. ID No. 1098 |
| 504 | Seq. ID No. 1099 | 251402 | 251372 | 251070 | Seq. ID No. 1100 |
| 505a | Seq. ID No. 1101 | 254277 | 254214 | 251794 | Seq. ID No. 1102 |
| 505b | Seq. ID No. 1103 | 254277 | 254268 | 251794 | Seq. ID No. 1104 |
| 507a | Seq. ID No. 1105 | 252725 | 252782 | 253009 | Seq. ID No. 1106 |
| 507b | Seq. ID No. 1107 | 252725 | 252743 | 253009 | Seq. ID No. 1108 |
| 509 | Seq. ID No. 1109 | 256252 | 256246 | 254297 | Seq. ID No. 1110 |
| 510 | Seq. ID No. 1111 | 254739 | 254781 | 255125 | Seq. ID No. 1112 |
| 512a | Seq. ID No. 1113 | 257457 | 257433 | 256297 | Seq. ID No. 1114 |
| 512b | Seq. ID No. 1115 | 257457 | 257442 | 256297 | Seq. ID No. 1116 |
| 514 | Seq. ID No. 1117 | 257684 | 257681 | 257439 | Seq. ID No. 1118 |
| 515a | Seq. ID No. 1119 | 259131 | 258687 | 257914 | Seq. ID No. 1120 |
| 515b | Seq. ID No. 1121 | 259131 | 258987 | 257914 | Seq. ID No. 1122 |
| 515c | Seq. ID No. 1123 | 259131 | 259116 | 257914 | Seq. ID No. 1124 |
| 518a | Seq. ID No. 1125 | 260687 | 260399 | 259284 | Seq. ID No. 1126 |
| 518b | Seq. ID No. 1127 | 260687 | 260636 | 259284 | Seq. ID No. 1128 |
| 518c | Seq. ID No. 1129 | 260687 | 260663 | 259284 | Seq. ID No. 1130 |
| 521b | Seq. ID No. 1131 | 260218 | 260218 | 259886 | Seq. ID No. 1132 |
| 523 | Seq. ID No. 1133 | 261324 | 261393 | 261749 | Seq. ID No. 1134 |
| 524a | Seq. ID No. 1135 | 261758 | 262406 | 262645 | Seq. ID No. 1136 |
| 524b | Seq. ID No. 1137 | 261758 | 261779 | 262645 | Seq. ID No. 1138 |
| 525 | Seq. ID No. 1139 | 262929 | 262920 | 262696 | Seq. ID No. 1140 |
| 527a | Seq. ID No. 1141 | 262700 | 262727 | 263797 | Seq. ID No. 1142 |
| 527b | Seq. ID No. 1143 | 262700 | 262709 | 263797 | Seq. ID No. 1144 |
| 528a | Seq. ID No. 1145 | 263837 | 263867 | 265153 | Seq. ID No. 1146 |
| 528b | Seq. ID No. 1147 | 263837 | 263861 | 265153 | Seq. ID No. 1148 |
| 529 | Seq. ID No. 1149 | 266200 | 266170 | 265265 | Seq. ID No. 1150 |
| 530a | Seq. ID No. 1151 | 265689 | 265725 | 266060 | Seq. ID No. 1152 |
| 530b | Seq. ID No. 1153 | 265689 | 265701 | 266060 | Seq. ID No. 1154 |
| 533 | Seq. ID No. 1155 | 266675 | 266675 | 268132 | Seq. ID No. 1156 |
| 535 | Seq. ID No. 1157 | 268203 | 268266 | 269903 | Seq. ID No. 1158 |
| 536 | Seq. ID No. 1159 | 269936 | 269951 | 270898 | Seq. ID No. 1160 |
| 539b | Seq. ID No. 1161 | 271377 | 271527 | 272060 | Seq. ID No. 1162 |
| 539c | Seq. ID No. 1163 | 271377 | 271392 | 272060 | Seq. ID No. 1164 |
| 541a | Seq. ID No. 1165 | 272061 | 272481 | 272726 | Seq. ID No. 1166 |
| 541b | Seq. ID No. 1167 | 272061 | 272301 | 272726 | Seq. ID No. 1168 |
| 541c | Seq. ID No. 1169 | 272061 | 272100 | 272726 | Seq. ID No. 1170 |
| 541d | Seq. ID No. 1171 | 272061 | 272073 | 272726 | Seq. ID No. 1172 |
| 542 | Seq. ID No. 1173 | 273208 | 273208 | 273047 | Seq. ID No. 1174 |
| 543a | Seq. ID No. 1175 | 273962 | 273872 | 273201 | Seq. ID No. 1176 |
| 543b | Seq. ID No. 1177 | 273962 | 273914 | 273201 | Seq. ID No. 1178 |
| 543c | Seq. ID No. 1179 | 273962 | 273923 | 273201 | Seq. ID No. 1180 |
| 544 | Seq. ID No. 1181 | 275016 | 275004 | 273883 | Seq. ID No. 1182 |
| 545a | Seq. ID No. 1183 | 276076 | 275425 | 275009 | Seq. ID No. 1184 |
| 545b | Seq. ID No. 1185 | 276076 | 275956 | 275009 | Seq. ID No. 1186 |
| 545c | Seq. ID No. 1187 | 276076 | 276064 | 275009 | Seq. ID No. 1188 |
| 546a | Seq. ID No. 1189 | 277005 | 276924 | 276061 | Seq. ID No. 1190 |
| 546b | Seq. ID No. 1191 | 277005 | 276996 | 276061 | Seq. ID No. 1192 |
| 546c | Seq. ID No. 1193 | 277005 | 277005 | 276061 | Seq. ID No. 1194 |
| 547a | Seq. ID No. 1195 | 277408 | 277321 | 277067 | Seq. ID No. 1196 |
| 547b | Seq. ID No. 1197 | 277408 | 277348 | 277067 | Seq. ID No. 1198 |
| 548 | Seq. ID No. 1199 | 277734 | 277737 | 278195 | Seq. ID No. 1200 |
| 549b | Seq. ID No. 1201 | 280434 | 280410 | 279952 | Seq. ID No. 1202 |
| 550a | Seq. ID No. 1203 | 280361 | 280289 | 280002 | Seq. ID No. 1204 |
| 551 | Seq. ID No. 1205 | 278647 | 278659 | 280005 | Seq. ID No. 1206 |
| 553c | Seq. ID No. 1207 | 280944 | 280935 | 280549 | Seq. ID No. 1208 |
| 554a | Seq. ID No. 1209 | 280006 | 280024 | 281070 | Seq. ID No. 1210 |
| 554b | Seq. ID No. 1211 | 280006 | 280018 | 281070 | Seq. ID No. 1212 |
| 556 | Seq. ID No. 1213 | 282361 | 282358 | 282101 | Seq. ID No. 1214 |
| 557a | Seq. ID No. 1215 | 281066 | 281096 | 282127 | Seq. ID No. 1216 |
| 557b | Seq. ID No. 1217 | 281066 | 281078 | 282127 | Seq. ID No. 1218 |
| 560a | Seq. ID No. 1219 | 282117 | 282180 | 283415 | Seq. ID No. 1220 |
| 560b | Seq. ID No. 1221 | 282117 | 282141 | 283415 | Seq. ID No. 1222 |
| 561 | Seq. ID No. 1223 | 285333 | 285315 | 283717 | Seq. ID No. 1224 |
| 563 | Seq. ID No. 1225 | 285391 | 285409 | 286263 | Seq. ID No. 1226 |
| 564a | Seq. ID No. 1227 | 287308 | 287116 | 286733 | Seq. ID No. 1228 |
| 564b | Seq. ID No. 1229 | 287308 | 287149 | 286733 | Seq. ID No. 1230 |
| 564c | Seq. ID No. 1231 | 287308 | 287305 | 286733 | Seq. ID No. 1232 |
| 569 | Seq. ID No. 1233 | 287443 | 287461 | 288639 | Seq. ID No. 1234 |
| 570a | Seq. ID No. 1235 | 290452 | 289936 | 288827 | Seq. ID No. 1236 |
| 570b | Seq. ID No. 1237 | 290452 | 290434 | 288827 | Seq. ID No. 1238 |
| 571 | Seq. ID No. 1239 | 288840 | 288888 | 289115 | Seq. ID No. 1240 |
| 573 | Seq. ID No. 1241 | 292365 | 292332 | 290620 | Seq. ID No. 1242 |
| 576a | Seq. ID No. 1243 | 293274 | 293223 | 292969 | Seq. ID No. 1244 |
| 576b | Seq. ID No. 1245 | 293274 | 293271 | 292969 | Seq. ID No. 1246 |
| 578a | Seq. ID No. 1247 | 293907 | 293826 | 293311 | Seq. ID No. 1248 |
| 578b | Seq. ID No. 1249 | 293907 | 293859 | 293311 | Seq. ID No. 1250 |
| 580a | Seq. ID No. 1251 | 292718 | 292730 | 294118 | Seq. ID No. 1252 |
| 580b | Seq. ID No. 1253 | 292718 | 292727 | 294118 | Seq. ID No. 1254 |
| 583a | Seq. ID No. 1255 | 294365 | 294470 | 296365 | Seq. ID No. 1256 |
| 583b | Seq. ID No. 1257 | 294365 | 294467 | 296365 | Seq. ID No. 1258 |
| 585b | Seq. ID No. 1259 | 297067 | 297028 | 296621 | Seq. ID No. 1260 |
| 586a | Seq. ID No. 1261 | 296610 | 296637 | 297083 | Seq. ID No. 1262 |
| 586b | Seq. ID No. 1263 | 296610 | 296613 | 297083 | Seq. ID No. 1264 |
| 587a | Seq. ID No. 1265 | 298917 | 298014 | 297532 | Seq. ID No. 1266 |
| 587b | Seq. ID No. 1267 | 298917 | 298155 | 297532 | Seq. ID No. 1268 |
| 587c | Seq. ID No. 1269 | 298917 | 298611 | 297532 | Seq. ID No. 1270 |
| 587d | Seq. ID No. 1271 | 298917 | 298884 | 297532 | Seq. ID No. 1272 |
| 588a | Seq. ID No. 1273 | 299041 | 298978 | 298235 | Seq. ID No. 1274 |
| 588b | Seq. ID No. 1275 | 299041 | 299008 | 298235 | Seq. ID No. 1276 |
| 589a | Seq. ID No. 1277 | 298358 | 298382 | 298864 | Seq. ID No. 1278 |
| 589b | Seq. ID No. 1279 | 298358 | 298364 | 298864 | Seq. ID No. 1280 |
| 590 | Seq. ID No. 1281 | 299156 | 299141 | 298887 | Seq. ID No. 1282 |
| 595a | Seq. ID No. 1283 | 304140 | 303912 | 303337 | Seq. ID No. 1284 |
| 595b | Seq. ID No. 1285 | 304140 | 304134 | 303337 | Seq. ID No. 1286 |
| 596a | Seq. ID No. 1287 | 303419 | 303536 | 303781 | Seq. ID No. 1288 |
| 596b | Seq. ID No. 1289 | 303419 | 303440 | 303781 | Seq. ID No. 1290 |
| 596c | Seq. ID No. 1291 | 303419 | 303434 | 303781 | Seq. ID No. 1292 |
| 597 | Seq. ID No. 1293 | 304680 | 304674 | 304141 | Seq. ID No. 1294 |
| 598a | Seq. ID No. 1295 | 303848 | 303869 | 304144 | Seq. ID No. 1296 |
| 598b | Seq. ID No. 1297 | 303848 | 303863 | 304144 | Seq. ID No. 1298 |
| 599a | Seq. ID No. 1299 | 305589 | 305412 | 304867 | Seq. ID No. 1300 |
| 599b | Seq. ID No. 1301 | 305589 | 305589 | 304867 | Seq. ID No. 1302 |
| 600a | Seq. ID No. 1303 | 304997 | 305018 | 305251 | Seq. ID No. 1304 |
| 600b | Seq. ID No. 1305 | 304997 | 305012 | 305251 | Seq. ID No. 1306 |
| 603a | Seq. ID No. 1307 | 306557 | 306572 | 306961 | Seq. ID No. 1308 |
| 606 | Seq. ID No. 1309 | 309046 | 309028 | 308543 | Seq. ID No. 1310 |
| 607a | Seq. ID No. 1311 | 307964 | 308051 | 308665 | Seq. ID No. 1312 |
| 607b | Seq. ID No. 1313 | 307964 | 307979 | 308665 | Seq. ID No. 1314 |
| 609a | Seq. ID No. 1315 | 299740 | 299791 | 309309 | Seq. ID No. 1316 |
| 609b | Seq. ID No. 1317 | 299740 | 299752 | 309309 | Seq. ID No. 1318 |
| 610a | Seq. ID No. 1319 | 310481 | 310454 | 310221 | Seq. ID No. 1320 |
| 610b | Seq. ID No. 1321 | 310481 | 310472 | 310221 | Seq. ID No. 1322 |
| 611 | Seq. ID No. 1323 | 310255 | 310258 | 310515 | Seq. ID No. 1324 |
| 613a | Seq. ID No. 1325 | 310512 | 310941 | 311675 | Seq. ID No. 1326 |
| 613b | Seq. ID No. 1327 | 310512 | 310749 | 311675 | Seq. ID No. 1328 |
| 613c | Seq. ID No. 1329 | 310512 | 310584 | 311675 | Seq. ID No. 1330 |
| 613d | Seq. ID No. 1331 | 310512 | 310512 | 311675 | Seq. ID No. 1332 |
| 614a | Seq. ID No. 1333 | 311715 | 311754 | 312194 | Seq. ID No. 1334 |
| 614b | Seq. ID No. 1335 | 311715 | 311736 | 312194 | Seq. ID No. 1336 |
| 615b | Seq. ID No. 1337 | 312828 | 312807 | 312418 | Seq. ID No. 1338 |
| 618 | Seq. ID No. 1339 | 312443 | 312443 | 313573 | Seq. ID No. 1340 |
| 619a | Seq. ID No. 1341 | 314156 | 314096 | 313866 | Seq. ID No. 1342 |
| 619b | Seq. ID No. 1343 | 314156 | 314123 | 313866 | Seq. ID No. 1344 |
| 620 | Seq. ID No. 1345 | 314583 | 314571 | 313969 | Seq. ID No. 1346 |
| 623 | Seq. ID No. 1347 | 315369 | 315318 | 314881 | Seq. ID No. 1348 |
| 624a | Seq. ID No. 1349 | 315050 | 315110 | 315355 | Seq. ID No. 1350 |
| 624b | Seq. ID No. 1351 | 315050 | 315107 | 315355 | Seq. ID No. 1352 |
| 624c | Seq. ID No. 1353 | 315050 | 315098 | 315355 | Seq. ID No. 1354 |
| 625 | Seq. ID No. 1355 | 315811 | 315811 | 315677 | Seq. ID No. 1356 |
| 626a | Seq. ID No. 1357 | 315560 | 315863 | 316420 | Seq. ID No. 1358 |
| 626b | Seq. ID No. 1359 | 315560 | 315638 | 316420 | Seq. ID No. 1360 |
| 626c | Seq. ID No. 1361 | 315560 | 315626 | 316420 | Seq. ID No. 1362 |
| 631a | Seq. ID No. 1363 | 316535 | 317297 | 319288 | Seq. ID No. 1364 |
| 631b | Seq. ID No. 1365 | 316535 | 316610 | 319288 | Seq. ID No. 1366 |
| 631c | Seq. ID No. 1367 | 316535 | 316574 | 319288 | Seq. ID No. 1368 |
| 631d | Seq. ID No. 1369 | 316535 | 316562 | 319288 | Seq. ID No. 1370 |
| 632b | Seq. ID No. 1371 | 319900 | 319867 | 319622 | Seq. ID No. 1372 |
| 633a | Seq. ID No. 1373 | 319626 | 319701 | 320822 | Seq. ID No. 1374 |
| 633b | Seq. ID No. 1375 | 319626 | 319680 | 320822 | Seq. ID No. 1376 |
| 633c | Seq. ID No. 1377 | 319626 | 319656 | 320822 | Seq. ID No. 1378 |
| 634b | Seq. ID No. 1379 | 320918 | 320921 | 321223 | Seq. ID No. 1380 |
| 636 | Seq. ID No. 1381 | 323400 | 323388 | 323161 | Seq. ID No. 1382 |
| 637a | Seq. ID No. 1383 | 321686 | 321737 | 323404 | Seq. ID No. 1384 |
| 637b | Seq. ID No. 1385 | 321686 | 321731 | 323404 | Seq. ID No. 1386 |
| 637c | Seq. ID No. 1387 | 321686 | 321695 | 323404 | Seq. ID No. 1388 |
| 640 | Seq. ID No. 1389 | 324722 | 324674 | 324414 | Seq. ID No. 1390 |
| 641a | Seq. ID No. 1391 | 323549 | 323594 | 324448 | Seq. ID No. 1392 |
| 641b | Seq. ID No. 1393 | 323549 | 323549 | 324448 | Seq. ID No. 1394 |
| 643 | Seq. ID No. 1395 | 324439 | 324460 | 324894 | Seq. ID No. 1396 |
| 644a | Seq. ID No. 1397 | 324791 | 324902 | 325087 | Seq. ID No. 1398 |
| 644b | Seq. ID No. 1399 | 324791 | 324830 | 325087 | Seq. ID No. 1400 |
| 645 | Seq. ID No. 1401 | 325456 | 325453 | 325181 | Seq. ID No. 1402 |
| 650 | Seq. ID No. 1403 | 325080 | 325125 | 326261 | Seq. ID No. 1404 |
| 652 | Seq. ID No. 1405 | 326348 | 326354 | 326716 | Seq. ID No. 1406 |
| 653a | Seq. ID No. 1407 | 326913 | 327024 | 328373 | Seq. ID No. 1408 |
| 653b | Seq. ID No. 1409 | 326913 | 326943 | 328373 | Seq. ID No. 1410 |
| 653c | Seq. ID No. 1411 | 326913 | 326925 | 328373 | Seq. ID No. 1412 |
| 656 | Seq. ID No. 1413 | 328370 | 328373 | 329863 | Seq. ID No. 1414 |
| 657c | Seq. ID No. 1415 | 330464 | 330530 | 330994 | Seq. ID No. 1416 |
| 658a | Seq. ID No. 1417 | 331110 | 331140 | 331565 | Seq. ID No. 1418 |
| 658b | Seq. ID No. 1419 | 331110 | 331110 | 331565 | Seq. ID No. 1420 |
| 659a | Seq. ID No. 1421 | 332594 | 332510 | 332055 | Seq. ID No. 1422 |
| 659b | Seq. ID No. 1423 | 332594 | 332582 | 332055 | Seq. ID No. 1424 |
| 661 | Seq. ID No. 1425 | 331974 | 332040 | 332771 | Seq. ID No. 1426 |
| 663 | Seq. ID No. 1427 | 333202 | 333205 | 333858 | Seq. ID No. 1428 |
| 665 | Seq. ID No. 1429 | 334188 | 334203 | 334598 | Seq. ID No. 1430 |
| 666 | Seq. ID No. 1431 | 334592 | 334598 | 334942 | Seq. ID No. 1432 |
| 670 | Seq. ID No. 1433 | 334988 | 335009 | 336517 | Seq. ID No. 1434 |
| 674a | Seq. ID No. 1435 | 336652 | 336691 | 338523 | Seq. ID No. 1436 |
| 674b | Seq. ID No. 1437 | 336652 | 336652 | 338523 | Seq. ID No. 1438 |
| 676 | Seq. ID No. 1439 | 340581 | 340611 | 340835 | Seq. ID No. 1440 |
| 677a | Seq. ID No. 1441 | 342484 | 342442 | 340949 | Seq. ID No. 1442 |
| 677b | Seq. ID No. 1443 | 342484 | 342469 | 340949 | Seq. ID No. 1444 |
| 679a | Seq. ID No. 1445 | 343500 | 343410 | 343186 | Seq. ID No. 1446 |
| 679b | Seq. ID No. 1447 | 343500 | 343461 | 343186 | Seq. ID No. 1448 |
| 680 | Seq. ID No. 1449 | 342758 | 342758 | 343474 | Seq. ID No. 1450 |
| 681 | Seq. ID No. 1451 | 343432 | 343540 | 344313 | Seq. ID No. 1452 |
| 683a | Seq. ID No. 1453 | 344288 | 344393 | 345187 | Seq. ID No. 1454 |
| 683b | Seq. ID No. 1455 | 344288 | 344294 | 345187 | Seq. ID No. 1456 |
| 684 | Seq. ID No. 1457 | 345386 | 345395 | 345622 | Seq. ID No. 1458 |
| 687 | Seq. ID No. 1459 | 345974 | 345974 | 347074 | Seq. ID No. 1460 |
| 688a | Seq. ID No. 1461 | 347062 | 347122 | 347847 | Seq. ID No. 1462 |
| 688b | Seq. ID No. 1463 | 347062 | 347116 | 347847 | Seq. ID No. 1464 |
| 690 | Seq. ID No. 1465 | 348033 | 348051 | 349016 | Seq. ID No. 1466 |
| 691a | Seq. ID No. 1467 | 349813 | 349777 | 349262 | Seq. ID No. 1468 |
| 691b | Seq. ID No. 1469 | 349813 | 349807 | 349262 | Seq. ID No. 1470 |
| 692 | Seq. ID No. 1471 | 350818 | 350797 | 350156 | Seq. ID No. 1472 |
| 694 | Seq. ID No. 1473 | 351167 | 351206 | 351940 | Seq. ID No. 1474 |
| 696 | Seq. ID No. 1475 | 351998 | 352013 | 352717 | Seq. ID No. 1476 |
| 698 | Seq. ID No. 1477 | 352953 | 352974 | 353339 | Seq. ID No. 1478 |
| 699b | Seq. ID No. 1479 | 354064 | 354055 | 353720 | Seq. ID No. 1480 |
| 700a | Seq. ID No. 1481 | 353340 | 353775 | 354068 | Seq. ID No. 1482 |
| 700b | Seq. ID No. 1483 | 353340 | 353364 | 354068 | Seq. ID No. 1484 |
| 700c | Seq. ID No. 1485 | 353340 | 353358 | 354068 | Seq. ID No. 1486 |
| 703 | Seq. ID No. 1487 | 354843 | 354864 | 356138 | Seq. ID No. 1488 |
| 705a | Seq. ID No. 1489 | 356139 | 356178 | 357137 | Seq. ID No. 1490 |
| 705b | Seq. ID No. 1491 | 356139 | 356151 | 357137 | Seq. ID No. 1492 |
| 707 | Seq. ID No. 1493 | 358618 | 358600 | 357662 | Seq. ID No. 1494 |
| 709 | Seq. ID No. 1495 | 359170 | 359134 | 358619 | Seq. ID No. 1496 |
| 710 | Seq. ID No. 1497 | 359781 | 359781 | 359350 | Seq. ID No. 1498 |
| 712a | Seq. ID No. 1499 | 360586 | 360520 | 359801 | Seq. ID No. 1500 |
| 712b | Seq. ID No. 1501 | 360586 | 360550 | 359801 | Seq. ID No. 1502 |
| 715a | Seq. ID No. 1503 | 360752 | 360839 | 361831 | Seq. ID No. 1504 |
| 715b | Seq. ID No. 1505 | 360752 | 360818 | 361831 | Seq. ID No. 1506 |
| 717 | Seq. ID No. 1507 | 361785 | 361833 | 362309 | Seq. ID No. 1508 |
| 718a | Seq. ID No. 1509 | 362257 | 362323 | 362622 | Seq. ID No. 1510 |
| 721 | Seq. ID No. 1511 | 363839 | 363815 | 363351 | Seq. ID No. 1512 |
| 722a | Seq. ID No. 1513 | 362629 | 363682 | 364056 | Seq. ID No. 1514 |
| 722b | Seq. ID No. 1515 | 362629 | 363304 | 364056 | Seq. ID No. 1516 |
| 722c | Seq. ID No. 1517 | 362629 | 362653 | 364056 | Seq. ID No. 1518 |
| 724a | Seq. ID No. 1519 | 364323 | 364773 | 366119 | Seq. ID No. 1520 |
| 724b | Seq. ID No. 1521 | 364323 | 364470 | 366119 | Seq. ID No. 1522 |
| 724c | Seq. ID No. 1523 | 364323 | 364455 | 366119 | Seq. ID No. 1524 |
| 724d | Seq. ID No. 1525 | 364323 | 364326 | 366119 | Seq. ID No. 1526 |
| 726 | Seq. ID No. 1527 | 366101 | 366155 | 366637 | Seq. ID No. 1528 |
| 728a | Seq. ID No. 1529 | 366642 | 366774 | 367577 | Seq. ID No. 1530 |
| 728b | Seq. ID No. 1531 | 366642 | 366654 | 367577 | Seq. ID No. 1532 |
| 730a | Seq. ID No. 1533 | 367564 | 368092 | 368385 | Seq. ID No. 1534 |
| 730b | Seq. ID No. 1535 | 367564 | 367888 | 368385 | Seq. ID No. 1536 |
| 730c | Seq. ID No. 1537 | 367564 | 367723 | 368385 | Seq. ID No. 1538 |
| 730d | Seq. ID No. 1539 | 367564 | 367564 | 368385 | Seq. ID No. 1540 |
| 731b | Seq. ID No. 1541 | 368400 | 368415 | 368798 | Seq. ID No. 1542 |
| 732 | Seq. ID No. 1543 | 368838 | 368880 | 370058 | Seq. ID No. 1544 |
| 738a | Seq. ID No. 1545 | 371023 | 372094 | 372465 | Seq. ID No. 1546 |
| 738b | Seq. ID No. 1547 | 371023 | 371140 | 372465 | Seq. ID No. 1548 |
| 738c | Seq. ID No. 1549 | 371023 | 371050 | 372465 | Seq. ID No. 1550 |
| 739 | Seq. ID No. 1551 | 373440 | 373434 | 373159 | Seq. ID No. 1552 |
| 740 | Seq. ID No. 1553 | 374954 | 374927 | 374634 | Seq. ID No. 1554 |
| 741a | Seq. ID No. 1555 | 378064 | 378016 | 377489 | Seq. ID No. 1556 |
| 741b | Seq. ID No. 1557 | 378064 | 378058 | 377489 | Seq. ID No. 1558 |
| 743a | Seq. ID No. 1559 | 372934 | 373003 | 378591 | Seq. ID No. 1560 |
| 743b | Seq. ID No. 1561 | 372934 | 372937 | 378591 | Seq. ID No. 1562 |
| 744 | Seq. ID No. 1563 | 379071 | 379080 | 380426 | Seq. ID No. 1564 |
| 745a | Seq. ID No. 1565 | 380454 | 380478 | 381371 | Seq. ID No. 1566 |
| 745b | Seq. ID No. 1567 | 380454 | 380463 | 381371 | Seq. ID No. 1568 |
| 746a | Seq. ID No. 1569 | 381346 | 381391 | 382191 | Seq. ID No. 1570 |
| 746b | Seq. ID No. 1571 | 381346 | 381364 | 382191 | Seq. ID No. 1572 |
| 747 | Seq. ID No. 1573 | 382184 | 382196 | 383323 | Seq. ID No. 1574 |
| 748a | Seq. ID No. 1575 | 383287 | 383437 | 384072 | Seq. ID No. 1576 |
| 748b | Seq. ID No. 1577 | 383287 | 383326 | 384072 | Seq. ID No. 1578 |
| 749 | Seq. ID No. 1579 | 386651 | 386615 | 386352 | Seq. ID No. 1580 |
| 751 | Seq. ID No. 1581 | 384408 | 384465 | 390965 | Seq. ID No. 1582 |
| 754a | Seq. ID No. 1583 | 391275 | 392166 | 392996 | Seq. ID No. 1584 |
| 754b | Seq. ID No. 1585 | 391275 | 391416 | 392996 | Seq. ID No. 1586 |
| 754c | Seq. ID No. 1587 | 391275 | 391335 | 392996 | Seq. ID No. 1588 |
| 754d | Seq. ID No. 1589 | 391275 | 391317 | 392996 | Seq. ID No. 1590 |
| 756a | Seq. ID No. 1591 | 393013 | 393040 | 394449 | Seq. ID No. 1592 |
| 756b | Seq. ID No. 1593 | 393013 | 393025 | 394449 | Seq. ID No. 1594 |
| 758 | Seq. ID No. 1595 | 394517 | 394532 | 394849 | Seq. ID No. 1596 |
| 760 | Seq. ID No. 1597 | 395517 | 395529 | 396824 | Seq. ID No. 1598 |
| 762a | Seq. ID No. 1599 | 396888 | 396909 | 397820 | Seq. ID No. 1600 |
| 762b | Seq. ID No. 1601 | 396888 | 396906 | 397820 | Seq. ID No. 1602 |
| 762c | Seq. ID No. 1603 | 396888 | 396903 | 397820 | Seq. ID No. 1604 |
| 763a | Seq. ID No. 1605 | 397821 | 397887 | 398228 | Seq. ID No. 1606 |
| 763b | Seq. ID No. 1607 | 397821 | 397824 | 398228 | Seq. ID No. 1608 |
| 764 | Seq. ID No. 1609 | 398537 | 398537 | 398635 | Seq. ID No. 1610 |
| 766 | Seq. ID No. 1611 | 399089 | 399101 | 399967 | Seq. ID No. 1612 |
| 767 | Seq. ID No. 1613 | 399968 | 400034 | 400435 | Seq. ID No. 1614 |
| 770a | Seq. ID No. 1615 | 400630 | 400984 | 401823 | Seq. ID No. 1616 |
| 770b | Seq. ID No. 1617 | 400630 | 400894 | 401823 | Seq. ID No. 1618 |
| 770c | Seq. ID No. 1619 | 400630 | 400693 | 401823 | Seq. ID No. 1620 |
| 772a | Seq. ID No. 1621 | 401846 | 401996 | 402577 | Seq. ID No. 1622 |
| 772b | Seq. ID No. 1623 | 401846 | 401849 | 402577 | Seq. ID No. 1624 |
| 774 | Seq. ID No. 1625 | 402606 | 402699 | 402929 | Seq. ID No. 1626 |
| 775 | Seq. ID No. 1627 | 402562 | 402595 | 403254 | Seq. ID No. 1628 |
| 777a | Seq. ID No. 1629 | 404283 | 404343 | 404675 | Seq. ID No. 1630 |
| 777c | Seq. ID No. 1631 | 404283 | 404298 | 404675 | Seq. ID No. 1632 |
| 779 | Seq. ID No. 1633 | 404803 | 404809 | 405120 | Seq. ID No. 1634 |
| 780a | Seq. ID No. 1635 | 404676 | 404748 | 405221 | Seq. ID No. 1636 |
| 780b | Seq. ID No. 1637 | 404676 | 404721 | 405221 | Seq. ID No. 1638 |
| 782b | Seq. ID No. 1639 | 406015 | 405988 | 405311 | Seq. ID No. 1640 |
| 785 | Seq. ID No. 1641 | 407774 | 407762 | 406443 | Seq. ID No. 1642 |
| 786b | Seq. ID No. 1643 | 407221 | 407152 | 406865 | Seq. ID No. 1644 |
| 788a | Seq. ID No. 1645 | 408557 | 408227 | 407775 | Seq. ID No. 1646 |
| 788b | Seq. ID No. 1647 | 408557 | 408557 | 407775 | Seq. ID No. 1648 |
| 789a | Seq. ID No. 1649 | 409361 | 409145 | 408558 | Seq. ID No. 1650 |
| 789b | Seq. ID No. 1651 | 409361 | 409259 | 408558 | Seq. ID No. 1652 |
| 789c | Seq. ID No. 1653 | 409361 | 409337 | 408558 | Seq. ID No. 1654 |
| 791 | Seq. ID No. 1655 | 408880 | 408910 | 409161 | Seq. ID No. 1656 |
| 792 | Seq. ID No. 1657 | 410107 | 410107 | 409340 | Seq. ID No. 1658 |
| 794a | Seq. ID No. 1659 | 411108 | 411066 | 410167 | Seq. ID No. 1660 |
| 794b | Seq. ID No. 1661 | 411108 | 411087 | 410167 | Seq. ID No. 1662 |
| 794c | Seq. ID No. 1663 | 411108 | 411096 | 410167 | Seq. ID No. 1664 |
| 794d | Seq. ID No. 1665 | 411108 | 411105 | 410167 | Seq. ID No. 1666 |
| 795 | Seq. ID No. 1667 | 411252 | 411252 | 411422 | Seq. ID No. 1668 |
| 797 | Seq. ID No. 1669 | 411604 | 411610 | 412293 | Seq. ID No. 1670 |
| 799a | Seq. ID No. 1671 | 412586 | 413474 | 413698 | Seq. ID No. 1672 |
| 799b | Seq. ID No. 1673 | 412586 | 413210 | 413698 | Seq. ID No. 1674 |
| 799c | Seq. ID No. 1675 | 412586 | 412757 | 413698 | Seq. ID No. 1676 |
| 799d | Seq. ID No. 1677 | 412586 | 412721 | 413698 | Seq. ID No. 1678 |
| 799e | Seq. ID No. 1679 | 412586 | 412667 | 413698 | Seq. ID No. 1680 |
| 799f | Seq. ID No. 1681 | 412586 | 412664 | 413698 | Seq. ID No. 1682 |
| 803a | Seq. ID No. 1683 | 413755 | 413779 | 415128 | Seq. ID No. 1684 |
| 803b | Seq. ID No. 1685 | 413755 | 413758 | 415128 | Seq. ID No. 1686 |
| 804a | Seq. ID No. 1687 | 415466 | 415487 | 416794 | Seq. ID No. 1688 |
| 804b | Seq. ID No. 1689 | 415466 | 415478 | 416794 | Seq. ID No. 1690 |
| 807 | Seq. ID No. 1691 | 416862 | 417024 | 418199 | Seq. ID No. 1692 |
| 808 | Seq. ID No. 1693 | 418549 | 418555 | 418794 | Seq. ID No. 1694 |
| 809b | Seq. ID No. 1695 | 419662 | 419632 | 419243 | Seq. ID No. 1696 |
| 812 | Seq. ID No. 1697 | 418200 | 418200 | 421274 | Seq. ID No. 1698 |
| 813 | Seq. ID No. 1699 | 421399 | 421414 | 422034 | Seq. ID No. 1700 |
| 816 | Seq. ID No. 1701 | 422122 | 422122 | 422283 | Seq. ID No. 1702 |
| 820a | Seq. ID No. 1703 | 424733 | 424739 | 424969 | Seq. ID No. 1704 |
| 820b | Seq. ID No. 1705 | 424733 | 424733 | 424969 | Seq. ID No. 1706 |
| 826 | Seq. ID No. 1707 | 422827 | 422842 | 428625 | Seq. ID No. 1708 |
| 829a | Seq. ID No. 1709 | 429410 | 429518 | 430123 | Seq. ID No. 1710 |
| 829b | Seq. ID No. 1711 | 429410 | 429416 | 430123 | Seq. ID No. 1712 |
| 830 | Seq. ID No. 1713 | 431650 | 431614 | 430421 | Seq. ID No. 1714 |
| 834a | Seq. ID No. 1715 | 431939 | 432029 | 432535 | Seq. ID No. 1716 |
| 834b | Seq. ID No. 1717 | 431939 | 431993 | 432535 | Seq. ID No. 1718 |
| 834c | Seq. ID No. 1719 | 431939 | 431972 | 432535 | Seq. ID No. 1720 |
| 835a | Seq. ID No. 1721 | 432638 | 432680 | 433231 | Seq. ID No. 1722 |
| 835b | Seq. ID No. 1723 | 432638 | 432644 | 433231 | Seq. ID No. 1724 |
| 836a | Seq. ID No. 1725 | 434079 | 434013 | 433579 | Seq. ID No. 1726 |
| 836b | Seq. ID No. 1727 | 434079 | 434034 | 433579 | Seq. ID No. 1728 |
| 836c | Seq. ID No. 1729 | 434079 | 434070 | 433579 | Seq. ID No. 1730 |
| 837a | Seq. ID No. 1731 | 434505 | 435360 | 435590 | Seq. ID No. 1732 |
| 837b | Seq. ID No. 1733 | 434505 | 434817 | 435590 | Seq. ID No. 1734 |
| 837c | Seq. ID No. 1735 | 434505 | 434505 | 435590 | Seq. ID No. 1736 |
| 843 | Seq. ID No. 1737 | 435813 | 435840 | 436859 | Seq. ID No. 1738 |
| 847a | Seq. ID No. 1739 | 436831 | 437089 | 439215 | Seq. ID No. 1740 |
| 847b | Seq. ID No. 1741 | 436831 | 437068 | 439215 | Seq. ID No. 1742 |
| 848 | Seq. ID No. 1743 | 439411 | 439411 | 439268 | Seq. ID No. 1744 |
| 849a | Seq. ID No. 1745 | 440732 | 440618 | 439470 | Seq. ID No. 1746 |
| 849b | Seq. ID No. 1747 | 440732 | 440684 | 439470 | Seq. ID No. 1748 |
| 851a | Seq. ID No. 1749 | 440982 | 441087 | 442634 | Seq. ID No. 1750 |
| 851b | Seq. ID No. 1751 | 440982 | 441072 | 442634 | Seq. ID No. 1752 |
| 853a | Seq. ID No. 1753 | 442726 | 442870 | 443319 | Seq. ID No. 1754 |
| 853b | Seq. ID No. 1755 | 442726 | 442753 | 443319 | Seq. ID No. 1756 |
| 853c | Seq. ID No. 1757 | 442726 | 442741 | 443319 | Seq. ID No. 1758 |
| 854 | Seq. ID No. 1759 | 444233 | 444233 | 444006 | Seq. ID No. 1760 |
| 855 | Seq. ID No. 1761 | 443652 | 443679 | 444119 | Seq. ID No. 1762 |
| 857 | Seq. ID No. 1763 | 446274 | 446247 | 445903 | Seq. ID No. 1764 |
| 859a | Seq. ID No. 1765 | 444185 | 445520 | 446704 | Seq. ID No. 1766 |
| 859b | Seq. ID No. 1767 | 444185 | 444869 | 446704 | Seq. ID No. 1768 |
| 859c | Seq. ID No. 1769 | 444185 | 444221 | 446704 | Seq. ID No. 1770 |
| 859d | Seq. ID No. 1771 | 444185 | 444212 | 446704 | Seq. ID No. 1772 |
| 860 | Seq. ID No. 1773 | 446857 | 446887 | 447093 | Seq. ID No. 1774 |
| 862 | Seq. ID No. 1775 | 447165 | 447171 | 447398 | Seq. ID No. 1776 |
| 863 | Seq. ID No. 1777 | 447152 | 447161 | 448180 | Seq. ID No. 1778 |
| 864a | Seq. ID No. 1779 | 449021 | 448991 | 448518 | Seq. ID No. 1780 |
| 864b | Seq. ID No. 1781 | 449021 | 449003 | 448518 | Seq. ID No. 1782 |
| 866a | Seq. ID No. 1783 | 448612 | 448801 | 449868 | Seq. ID No. 1784 |
| 866b | Seq. ID No. 1785 | 448612 | 448612 | 449868 | Seq. ID No. 1786 |
| 868a | Seq. ID No. 1787 | 449855 | 450269 | 450718 | Seq. ID No. 1788 |
| 868b | Seq. ID No. 1789 | 449855 | 449861 | 450718 | Seq. ID No. 1790 |
| 870 | Seq. ID No. 1791 | 450768 | 450792 | 451754 | Seq. ID No. 1792 |
| 873a | Seq. ID No. 1793 | 451842 | 452103 | 452924 | Seq. ID No. 1794 |
| 873b | Seq. ID No. 1795 | 451842 | 451920 | 452924 | Seq. ID No. 1796 |
| 873c | Seq. ID No. 1797 | 451842 | 451866 | 452924 | Seq. ID No. 1798 |
| 874a | Seq. ID No. 1799 | 453019 | 453136 | 453336 | Seq. ID No. 1800 |
| 874b | Seq. ID No. 1801 | 453019 | 453043 | 453336 | Seq. ID No. 1802 |
| 875 | Seq. ID No. 1803 | 453388 | 453415 | 453651 | Seq. ID No. 1804 |
| 876 | Seq. ID No. 1805 | 453874 | 453898 | 454509 | Seq. ID No. 1806 |
| 878a | Seq. ID No. 1807 | 454510 | 454738 | 455313 | Seq. ID No. 1808 |
| 878b | Seq. ID No. 1809 | 454510 | 454588 | 455313 | Seq. ID No. 1810 |
| 878c | Seq. ID No. 1811 | 454510 | 454549 | 455313 | Seq. ID No. 1812 |
| 880a | Seq. ID No. 1813 | 455379 | 455454 | 456086 | Seq. ID No. 1814 |
| 880b | Seq. ID No. 1815 | 455379 | 455406 | 456086 | Seq. ID No. 1816 |
| 880c | Seq. ID No. 1817 | 455379 | 455397 | 456086 | Seq. ID No. 1818 |
| 885 | Seq. ID No. 1819 | 456297 | 456309 | 457472 | Seq. ID No. 1820 |
| 886 | Seq. ID No. 1821 | 457408 | 457453 | 458769 | Seq. ID No. 1822 |
| 887 | Seq. ID No. 1823 | 459402 | 459396 | 459118 | Seq. ID No. 1824 |
| 888 | Seq. ID No. 1825 | 459077 | 459080 | 460357 | Seq. ID No. 1826 |
| 890a | Seq. ID No. 1827 | 460448 | 460523 | 461002 | Seq. ID No. 1828 |
| 890b | Seq. ID No. 1829 | 460448 | 460475 | 461002 | Seq. ID No. 1830 |
| 894 | Seq. ID No. 1831 | 463346 | 463346 | 463140 | Seq. ID No. 1832 |
| 904 | Seq. ID No. 1833 | 468103 | 468097 | 467843 | Seq. ID No. 1834 |
| 907a | Seq. ID No. 1835 | 470121 | 470022 | 469240 | Seq. ID No. 1836 |
| 907b | Seq. ID No. 1837 | 470121 | 470103 | 469240 | Seq. ID No. 1838 |
| 908a | Seq. ID No. 1839 | 471898 | 471376 | 470636 | Seq. ID No. 1840 |
| 908b | Seq. ID No. 1841 | 471898 | 471886 | 470636 | Seq. ID No. 1842 |
| 908c | Seq. ID No. 1843 | 471898 | 471895 | 470636 | Seq. ID No. 1844 |
| 911 | Seq. ID No. 1845 | 472251 | 472314 | 472943 | Seq. ID No. 1846 |
| 912 | Seq. ID No. 1847 | 472944 | 472953 | 473780 | Seq. ID No. 1848 |
| 916 | Seq. ID No. 1849 | 473774 | 473798 | 476413 | Seq. ID No. 1850 |
| 917 | Seq. ID No. 1851 | 476720 | 476726 | 476983 | Seq. ID No. 1852 |
| 918b | Seq. ID No. 1853 | 476974 | 477043 | 477636 | Seq. ID No. 1854 |
| 918c | Seq. ID No. 1855 | 476974 | 476986 | 477636 | Seq. ID No. 1856 |
| 919 | Seq. ID No. 1857 | 477670 | 477709 | 478032 | Seq. ID No. 1858 |
| 923 | Seq. ID No. 1859 | 477585 | 477636 | 479852 | Seq. ID No. 1860 |
| 925a | Seq. ID No. 1861 | 479863 | 480316 | 481326 | Seq. ID No. 1862 |
| 925b | Seq. ID No. 1863 | 479863 | 479887 | 481326 | Seq. ID No. 1864 |
| 926 | Seq. ID No. 1865 | 481268 | 481319 | 482335 | Seq. ID No. 1866 |
| 927 | Seq. ID No. 1867 | 482317 | 482347 | 482928 | Seq. ID No. 1868 |
| 929a | Seq. ID No. 1869 | 482918 | 484175 | 484519 | Seq. ID No. 1870 |
| 929b | Seq. ID No. 1871 | 482918 | 482918 | 484519 | Seq. ID No. 1872 |
| 930 | Seq. ID No. 1873 | 484543 | 484543 | 485790 | Seq. ID No. 1874 |
| 931 | Seq. ID No. 1875 | 486281 | 486221 | 485898 | Seq. ID No. 1876 |
| 932 | Seq. ID No. 1877 | 485791 | 485809 | 486294 | Seq. ID No. 1878 |
| 934a | Seq. ID No. 1879 | 486257 | 486377 | 487435 | Seq. ID No. 1880 |
| 934b | Seq. ID No. 1881 | 486257 | 486353 | 487435 | Seq. ID No. 1882 |
| 934c | Seq. ID No. 1883 | 486257 | 486278 | 487435 | Seq. ID No. 1884 |
| 934d | Seq. ID No. 1885 | 486257 | 486269 | 487435 | Seq. ID No. 1886 |
| 935 | Seq. ID No. 1887 | 487436 | 487463 | 488152 | Seq. ID No. 1888 |
| 938 | Seq. ID No. 1889 | 488336 | 488399 | 489727 | Seq. ID No. 1890 |
| 939a | Seq. ID No. 1891 | 491177 | 490841 | 489834 | Seq. ID No. 1892 |
| 939b | Seq. ID No. 1893 | 491177 | 490982 | 489834 | Seq. ID No. 1894 |
| 939c | Seq. ID No. 1895 | 491177 | 491162 | 489834 | Seq. ID No. 1896 |
| 942 | Seq. ID No. 1897 | 492089 | 492065 | 491685 | Seq. ID No. 1898 |
| 943 | Seq. ID No. 1899 | 492484 | 492466 | 492164 | Seq. ID No. 1900 |
| 944a | Seq. ID No. 1901 | 493707 | 493518 | 492457 | Seq. ID No. 1902 |
| 944b | Seq. ID No. 1903 | 493707 | 493593 | 492457 | Seq. ID No. 1904 |
| 946 | Seq. ID No. 1905 | 493834 | 493834 | 494376 | Seq. ID No. 1906 |
| 949a | Seq. ID No. 1907 | 494411 | 494837 | 495802 | Seq. ID No. 1908 |
| 949b | Seq. ID No. 1909 | 494411 | 494537 | 495802 | Seq. ID No. 1910 |
| 949c | Seq. ID No. 1911 | 494411 | 494498 | 495802 | Seq. ID No. 1912 |
| 949d | Seq. ID No. 1913 | 494411 | 494435 | 495802 | Seq. ID No. 1914 |
| 950 | Seq. ID No. 1915 | 496174 | 496171 | 495929 | Seq. ID No. 1916 |
| 951a | Seq. ID No. 1917 | 495906 | 495984 | 496895 | Seq. ID No. 1918 |
| 951b | Seq. ID No. 1919 | 495906 | 495915 | 496895 | Seq. ID No. 1920 |
| 952a | Seq. ID No. 1921 | 497115 | 497547 | 498344 | Seq. ID No. 1922 |
| 952b | Seq. ID No. 1923 | 497115 | 497211 | 498344 | Seq. ID No. 1924 |
| 954 | Seq. ID No. 1925 | 498503 | 498518 | 499984 | Seq. ID No. 1926 |
| 955a | Seq. ID No. 1927 | 500596 | 500569 | 500297 | Seq. ID No. 1928 |
| 955b | Seq. ID No. 1929 | 500596 | 500575 | 500297 | Seq. ID No. 1930 |
| 956 | Seq. ID No. 1931 | 500019 | 500019 | 501308 | Seq. ID No. 1932 |
| 958a | Seq. ID No. 1933 | 501309 | 501417 | 501866 | Seq. ID No. 1934 |
| 958b | Seq. ID No. 1935 | 501309 | 501309 | 501866 | Seq. ID No. 1936 |
| 959 | Seq. ID No. 1937 | 501978 | 501993 | 502364 | Seq. ID No. 1938 |
| 961a | Seq. ID No. 1939 | 502392 | 502446 | 502943 | Seq. ID No. 1940 |
| 961b | Seq. ID No. 1941 | 502392 | 502425 | 502943 | Seq. ID No. 1942 |
| 961c | Seq. ID No. 1943 | 502392 | 502413 | 502943 | Seq. ID No. 1944 |
| 963 | Seq. ID No. 1945 | 502951 | 502978 | 503949 | Seq. ID No. 1946 |
| 965a | Seq. ID No. 1947 | 503950 | 503974 | 504930 | Seq. ID No. 1948 |
| 965b | Seq. ID No. 1949 | 503950 | 503965 | 504930 | Seq. ID No. 1950 |
| 966a | Seq. ID No. 1951 | 504879 | 504990 | 506417 | Seq. ID No. 1952 |
| 966b | Seq. ID No. 1953 | 504879 | 504975 | 506417 | Seq. ID No. 1954 |
| 967a | Seq. ID No. 1955 | 506365 | 506407 | 506805 | Seq. ID No. 1956 |
| 967b | Seq. ID No. 1957 | 506365 | 506377 | 506805 | Seq. ID No. 1958 |
| 968 | Seq. ID No. 1959 | 507166 | 507184 | 507951 | Seq. ID No. 1960 |
| 969 | Seq. ID No. 1961 | 510913 | 510913 | 508775 | Seq. ID No. 1962 |
| 972a | Seq. ID No. 1963 | 512618 | 512444 | 511044 | Seq. ID No. 1964 |
| 972b | Seq. ID No. 1965 | 512618 | 512603 | 511044 | Seq. ID No. 1966 |
| 976 | Seq. ID No. 1967 | 512815 | 512863 | 514659 | Seq. ID No. 1968 |
| 977a | Seq. ID No. 1969 | 515753 | 515654 | 514809 | Seq. ID No. 1970 |
| 977b | Seq. ID No. 1971 | 515753 | 515738 | 514809 | Seq. ID No. 1972 |
| 978a | Seq. ID No. 1973 | 516892 | 516718 | 516326 | Seq. ID No. 1974 |
| 978b | Seq. ID No. 1975 | 516892 | 516856 | 516326 | Seq. ID No. 1976 |
| 979 | Seq. ID No. 1977 | 517178 | 517178 | 517002 | Seq. ID No. 1978 |
| 980 | Seq. ID No. 1979 | 518090 | 518063 | 517239 | Seq. ID No. 1980 |
| 981a | Seq. ID No. 1981 | 517537 | 517717 | 517962 | Seq. ID No. 1982 |
| 982 | Seq. ID No. 1983 | 519084 | 519033 | 518146 | Seq. ID No. 1984 |
| 984a | Seq. ID No. 1985 | 519141 | 519309 | 519995 | Seq. ID No. 1986 |
| 984b | Seq. ID No. 1987 | 519141 | 519216 | 519995 | Seq. ID No. 1988 |
| 986 | Seq. ID No. 1989 | 520057 | 520066 | 521073 | Seq. ID No. 1990 |
| 987 | Seq. ID No. 1991 | 521626 | 521614 | 521147 | Seq. ID No. 1992 |
| 988a | Seq. ID No. 1993 | 522445 | 522070 | 521627 | Seq. ID No. 1994 |
| 988b | Seq. ID No. 1995 | 522445 | 522328 | 521627 | Seq. ID No. 1996 |
| 988c | Seq. ID No. 1997 | 522445 | 522391 | 521627 | Seq. ID No. 1998 |
| 988d | Seq. ID No. 1999 | 522445 | 522433 | 521627 | Seq. ID No. 2000 |
| 991 | Seq. ID No. 2001 | 522779 | 522782 | 523339 | Seq. ID No. 2002 |
| 992 | Seq. ID No. 2003 | 524472 | 524454 | 523420 | Seq. ID No. 2004 |
| 993 | Seq. ID No. 2005 | 523372 | 523387 | 524031 | Seq. ID No. 2006 |
| 994 | Seq. ID No. 2007 | 523709 | 523763 | 524269 | Seq. ID No. 2008 |
| 995 | Seq. ID No. 2009 | 524843 | 524789 | 524508 | Seq. ID No. 2010 |
| 997 | Seq. ID No. 2011 | 525316 | 525151 | 525044 | Seq. ID No. 2012 |
| 998a | Seq. ID No. 2013 | 524933 | 524999 | 525658 | Seq. ID No. 2014 |
| 998b | Seq. ID No. 2015 | 524933 | 524939 | 525658 | Seq. ID No. 2016 |
| 999a | Seq. ID No. 2017 | 526067 | 526016 | 525789 | Seq. ID No. 2018 |
| 999b | Seq. ID No. 2019 | 526067 | 526058 | 525789 | Seq. ID No. 2020 |
| 1000 | Seq. ID No. 2021 | 526066 | 526141 | 526833 | Seq. ID No. 2022 |
| 1001 | Seq. ID No. 2023 | 526853 | 526853 | 527035 | Seq. ID No. 2024 |
| 1003a | Seq. ID No. 2025 | 529482 | 529476 | 529228 | Seq. ID No. 2026 |
| 1003b | Seq. ID No. 2027 | 529482 | 529482 | 529228 | Seq. ID No. 2028 |
| 1004a | Seq. ID No. 2029 | 527320 | 527350 | 529245 | Seq. ID No. 2030 |
| 1004b | Seq. ID No. 2031 | 527320 | 527335 | 529245 | Seq. ID No. 2032 |
| 1006a | Seq. ID No. 2033 | 529232 | 529352 | 530305 | Seq. ID No. 2034 |
| 1006b | Seq. ID No. 2035 | 529232 | 529289 | 530305 | Seq. ID No. 2036 |
| 1007 | Seq. ID No. 2037 | 531623 | 531593 | 530370 | Seq. ID No. 2038 |
| 1012 | Seq. ID No. 2039 | 531856 | 531856 | 531972 | Seq. ID No. 2040 |
| 1013a | Seq. ID No. 2041 | 532239 | 532698 | 533411 | Seq. ID No. 2042 |
| 1013b | Seq. ID No. 2043 | 532239 | 532467 | 533411 | Seq. ID No. 2044 |
| 1013c | Seq. ID No. 2045 | 532239 | 532359 | 533411 | Seq. ID No. 2046 |
| 1016c | Seq. ID No. 2047 | 533402 | 533432 | 534148 | Seq. ID No. 2048 |
| 1017a | Seq. ID No. 2049 | 534103 | 534754 | 535266 | Seq. ID No. 2050 |
| 1017b | Seq. ID No. 2051 | 534103 | 534274 | 535266 | Seq. ID No. 2052 |
| 1017c | Seq. ID No. 2053 | 534103 | 534187 | 535266 | Seq. ID No. 2054 |
| 1017d | Seq. ID No. 2055 | 534103 | 534145 | 535266 | Seq. ID No. 2056 |
| 1018a | Seq. ID No. 2057 | 535957 | 535981 | 536787 | Seq. ID No. 2058 |
| 1018b | Seq. ID No. 2059 | 535957 | 535978 | 536787 | Seq. ID No. 2060 |
| 1021a | Seq. ID No. 2061 | 536726 | 536804 | 537688 | Seq. ID No. 2062 |
| 1021b | Seq. ID No. 2063 | 536726 | 536750 | 537688 | Seq. ID No. 2064 |
| 1021c | Seq. ID No. 2065 | 536726 | 536729 | 537688 | Seq. ID No. 2066 |
| 1024 | Seq. ID No. 2067 | 537700 | 537700 | 538749 | Seq. ID No. 2068 |
| 1025 | Seq. ID No. 2069 | 538763 | 538772 | 539779 | Seq. ID No. 2070 |
| 1026 | Seq. ID No. 2071 | 541222 | 541222 | 539954 | Seq. ID No. 2072 |
| 1029 | Seq. ID No. 2073 | 541559 | 541619 | 542479 | Seq. ID No. 2074 |
| 1033a | Seq. ID No. 2075 | 542702 | 542966 | 544783 | Seq. ID No. 2076 |
| 1033b | Seq. ID No. 2077 | 542702 | 542825 | 544783 | Seq. ID No. 2078 |
| 1033c | Seq. ID No. 2079 | 542702 | 542741 | 544783 | Seq. ID No. 2080 |
| 1035a | Seq. ID No. 2081 | 546793 | 546721 | 546416 | Seq. ID No. 2082 |
| 1035b | Seq. ID No. 2083 | 546793 | 546724 | 546416 | Seq. ID No. 2084 |
| 1035c | Seq. ID No. 2085 | 546793 | 546739 | 546416 | Seq. ID No. 2086 |
| 1036a | Seq. ID No. 2087 | 544924 | 545824 | 546423 | Seq. ID No. 2088 |
| 1036b | Seq. ID No. 2089 | 544924 | 544978 | 546423 | Seq. ID No. 2090 |
| 1036c | Seq. ID No. 2091 | 544924 | 544927 | 546423 | Seq. ID No. 2092 |
| 1037 | Seq. ID No. 2093 | 546420 | 546423 | 547151 | Seq. ID No. 2094 |
| 1039a | Seq. ID No. 2095 | 547314 | 547344 | 548114 | Seq. ID No. 2096 |
| 1039b | Seq. ID No. 2097 | 547314 | 547317 | 548114 | Seq. ID No. 2098 |
| 1041 | Seq. ID No. 2099 | 548111 | 548132 | 548725 | Seq. ID No. 2100 |
| 1042a | Seq. ID No. 2101 | 548729 | 548750 | 549646 | Seq. ID No. 2102 |
| 1042b | Seq. ID No. 2103 | 548729 | 548729 | 549646 | Seq. ID No. 2104 |
| 1044a | Seq. ID No. 2105 | 551333 | 551243 | 550767 | Seq. ID No. 2106 |
| 1044b | Seq. ID No. 2107 | 551333 | 551306 | 550767 | Seq. ID No. 2108 |
| 1044c | Seq. ID No. 2109 | 551333 | 551312 | 550767 | Seq. ID No. 2110 |
| 1044d | Seq. ID No. 2111 | 551333 | 551324 | 550767 | Seq. ID No. 2112 |
| 1047a | Seq. ID No. 2113 | 550332 | 550395 | 552098 | Seq. ID No. 2114 |
| 1047b | Seq. ID No. 2115 | 550332 | 550374 | 552098 | Seq. ID No. 2116 |
| 1049a | Seq. ID No. 2117 | 552053 | 552182 | 552889 | Seq. ID No. 2118 |
| 1049b | Seq. ID No. 2119 | 552053 | 552110 | 552889 | Seq. ID No. 2120 |
| 1050a | Seq. ID No. 2121 | 552811 | 553336 | 553635 | Seq. ID No. 2122 |
| 1050b | Seq. ID No. 2123 | 552811 | 552859 | 553635 | Seq. ID No. 2124 |
| 1050c | Seq. ID No. 2125 | 552811 | 552841 | 553635 | Seq. ID No. 2126 |
| 1050d | Seq. ID No. 2127 | 552811 | 552835 | 553635 | Seq. ID No. 2128 |
| 1055 | Seq. ID No. 2129 | 557181 | 557130 | 556642 | Seq. ID No. 2130 |
| 1056a | Seq. ID No. 2131 | 556313 | 556367 | 556645 | Seq. ID No. 2132 |
| 1056b | Seq. ID No. 2133 | 556313 | 556364 | 556645 | Seq. ID No. 2134 |
| 1057 | Seq. ID No. 2135 | 553741 | 553774 | 557208 | Seq. ID No. 2136 |
| 1058 | Seq. ID No. 2137 | 557389 | 557389 | 558231 | Seq. ID No. 2138 |
| 1060a | Seq. ID No. 2139 | 558338 | 558434 | 559720 | Seq. ID No. 2140 |
| 1060b | Seq. ID No. 2141 | 558338 | 558353 | 559720 | Seq. ID No. 2142 |
| 1062 | Seq. ID No. 2143 | 559708 | 559717 | 560406 | Seq. ID No. 2144 |
| 1063 | Seq. ID No. 2145 | 560912 | 560888 | 560631 | Seq. ID No. 2146 |
| 1064 | Seq. ID No. 2147 | 560346 | 560415 | 560714 | Seq. ID No. 2148 |
| 1065a | Seq. ID No. 2149 | 560743 | 560857 | 561312 | Seq. ID No. 2150 |
| 1065b | Seq. ID No. 2151 | 560743 | 560824 | 561312 | Seq. ID No. 2152 |
| 1065c | Seq. ID No. 2153 | 560743 | 560791 | 561312 | Seq. ID No. 2154 |
| 1066a | Seq. ID No. 2155 | 561721 | 561637 | 561359 | Seq. ID No. 2156 |
| 1066b | Seq. ID No. 2157 | 561721 | 561715 | 561359 | Seq. ID No. 2158 |
| 1067 | Seq. ID No. 2159 | 561384 | 561417 | 562028 | Seq. ID No. 2160 |
| 1069 | Seq. ID No. 2161 | 562186 | 562186 | 562629 | Seq. ID No. 2162 |
| 1070a | Seq. ID No. 2163 | 562626 | 562911 | 563282 | Seq. ID No. 2164 |
| 1070b | Seq. ID No. 2165 | 562626 | 562635 | 563282 | Seq. ID No. 2166 |
| 1076a | Seq. ID No. 2167 | 563437 | 563620 | 566925 | Seq. ID No. 2168 |
| 1076b | Seq. ID No. 2169 | 563437 | 563443 | 566925 | Seq. ID No. 2170 |
| 1077a | Seq. ID No. 2171 | 566894 | 568136 | 568525 | Seq. ID No. 2172 |
| 1077b | Seq. ID No. 2173 | 566894 | 567506 | 568525 | Seq. ID No. 2174 |
| 1077c | Seq. ID No. 2175 | 566894 | 567296 | 568525 | Seq. ID No. 2176 |
| 1077d | Seq. ID No. 2177 | 566894 | 567170 | 568525 | Seq. ID No. 2178 |
| 1077e | Seq. ID No. 2179 | 566894 | 566918 | 568525 | Seq. ID No. 2180 |
| 1079a | Seq. ID No. 2181 | 568458 | 568518 | 569591 | Seq. ID No. 2182 |
| 1079b | Seq. ID No. 2183 | 568458 | 568485 | 569591 | Seq. ID No. 2184 |
| 1080a | Seq. ID No. 2185 | 569461 | 569575 | 569865 | Seq. ID No. 2186 |
| 1081a | Seq. ID No. 2187 | 570009 | 570099 | 570491 | Seq. ID No. 2188 |
| 1081b | Seq. ID No. 2189 | 570009 | 570021 | 570491 | Seq. ID No. 2190 |
| 1082 | Seq. ID No. 2191 | 570545 | 570560 | 571390 | Seq. ID No. 2192 |
| 1085 | Seq. ID No. 2193 | 571448 | 571493 | 571930 | Seq. ID No. 2194 |
| 1086 | Seq. ID No. 2195 | 572006 | 572021 | 573433 | Seq. ID No. 2196 |
| 1087a | Seq. ID No. 2197 | 573393 | 573438 | 573983 | Seq. ID No. 2198 |
| 1087b | Seq. ID No. 2199 | 573393 | 573402 | 573983 | Seq. ID No. 2200 |
| 1091a | Seq. ID No. 2201 | 574840 | 574903 | 575133 | Seq. ID No. 2202 |
| 1091b | Seq. ID No. 2203 | 574840 | 574888 | 575133 | Seq. ID No. 2204 |
| 1094a | Seq. ID No. 2205 | 574083 | 574122 | 576380 | Seq. ID No. 2206 |
| 1094b | Seq. ID No. 2207 | 574083 | 574110 | 576380 | Seq. ID No. 2208 |
| 1095a | Seq. ID No. 2209 | 575929 | 575968 | 576420 | Seq. ID No. 2210 |
| 1095b | Seq. ID No. 2211 | 575929 | 575950 | 576420 | Seq. ID No. 2212 |
| 1096a | Seq. ID No. 2213 | 576567 | 576657 | 577469 | Seq. ID No. 2214 |
| 1096b | Seq. ID No. 2215 | 576567 | 576597 | 577469 | Seq. ID No. 2216 |
| 1096c | Seq. ID No. 2217 | 576567 | 576585 | 577469 | Seq. ID No. 2218 |
| 1097 | Seq. ID No. 2219 | 577758 | 577839 | 578270 | Seq. ID No. 2220 |
| 1098b | Seq. ID No. 2221 | 578304 | 578304 | 578753 | Seq. ID No. 2222 |
| 1100 | Seq. ID No. 2223 | 578719 | 578740 | 579318 | Seq. ID No. 2224 |
| 1101a | Seq. ID No. 2225 | 579319 | 579388 | 579663 | Seq. ID No. 2226 |
| 1101b | Seq. ID No. 2227 | 579319 | 579325 | 579663 | Seq. ID No. 2228 |
| 1104 | Seq. ID No. 2229 | 579892 | 579925 | 580959 | Seq. ID No. 2230 |
| 1106 | Seq. ID No. 2231 | 581018 | 581033 | 581344 | Seq. ID No. 2232 |
| 1108a | Seq. ID No. 2233 | 581194 | 581413 | 582546 | Seq. ID No. 2234 |
| 1108b | Seq. ID No. 2235 | 581194 | 581254 | 582546 | Seq. ID No. 2236 |
| 1111a | Seq. ID No. 2237 | 582575 | 582815 | 583843 | Seq. ID No. 2238 |
| 1111b | Seq. ID No. 2239 | 582575 | 582611 | 583843 | Seq. ID No. 2240 |
| 1111c | Seq. ID No. 2241 | 582575 | 582578 | 583843 | Seq. ID No. 2242 |
| 1112a | Seq. ID No. 2243 | 584607 | 584424 | 584239 | Seq. ID No. 2244 |
| 1125 | Seq. ID No. 2245 | 590309 | 590312 | 590752 | Seq. ID No. 2246 |
| 1126a | Seq. ID No. 2247 | 590674 | 590830 | 591966 | Seq. ID No. 2248 |
| 1126b | Seq. ID No. 2249 | 590674 | 590749 | 591966 | Seq. ID No. 2250 |
| 1126c | Seq. ID No. 2251 | 590674 | 590734 | 591966 | Seq. ID No. 2252 |
| 1127 | Seq. ID No. 2253 | 591927 | 591963 | 592610 | Seq. ID No. 2254 |
| 1128 | Seq. ID No. 2255 | 592612 | 592612 | 592767 | Seq. ID No. 2256 |
| 1131 | Seq. ID No. 2257 | 593102 | 593108 | 594118 | Seq. ID No. 2258 |
| 1132 | Seq. ID No. 2259 | 594208 | 594211 | 595179 | Seq. ID No. 2260 |
| 1135a | Seq. ID No. 2261 | 595339 | 595396 | 596805 | Seq. ID No. 2262 |
| 1135b | Seq. ID No. 2263 | 595339 | 595381 | 596805 | Seq. ID No. 2264 |
| 1136a | Seq. ID No. 2265 | 598288 | 598243 | 596903 | Seq. ID No. 2266 |
| 1136b | Seq. ID No. 2267 | 598288 | 598267 | 596903 | Seq. ID No. 2268 |
| 1138 | Seq. ID No. 2269 | 599921 | 599849 | 598668 | Seq. ID No. 2270 |
| 1140a | Seq. ID No. 2271 | 602187 | 601377 | 600247 | Seq. ID No. 2272 |
| 1140b | Seq. ID No. 2273 | 602187 | 601914 | 600247 | Seq. ID No. 2274 |
| 1140c | Seq. ID No. 2275 | 602187 | 602187 | 600247 | Seq. ID No. 2276 |
| 1141a | Seq. ID No. 2277 | 600251 | 600302 | 600556 | Seq. ID No. 2278 |
| 1141b | Seq. ID No. 2279 | 600251 | 600257 | 600556 | Seq. ID No. 2280 |
| 1142 | Seq. ID No. 2281 | 602718 | 602700 | 602377 | Seq. ID No. 2282 |
| 1144a | Seq. ID No. 2283 | 602866 | 603421 | 603672 | Seq. ID No. 2284 |
| 1144b | Seq. ID No. 2285 | 602866 | 602962 | 603672 | Seq. ID No. 2286 |
| 1144c | Seq. ID No. 2287 | 602866 | 602893 | 603672 | Seq. ID No. 2288 |
| 1146a | Seq. ID No. 2289 | 603660 | 604296 | 604598 | Seq. ID No. 2290 |
| 1146b | Seq. ID No. 2291 | 603660 | 604140 | 604598 | Seq. ID No. 2292 |
| 1146c | Seq. ID No. 2293 | 603660 | 603963 | 604598 | Seq. ID No. 2294 |
| 1146d | Seq. ID No. 2295 | 603660 | 603828 | 604598 | Seq. ID No. 2296 |
| 1146e | Seq. ID No. 2297 | 603660 | 603675 | 604598 | Seq. ID No. 2298 |
| 1148 | Seq. ID No. 2299 | 604610 | 604631 | 605278 | Seq. ID No. 2300 |
| 1149 | Seq. ID No. 2301 | 607200 | 607170 | 605347 | Seq. ID No. 2302 |
| 1152a | Seq. ID No. 2303 | 607234 | 608383 | 608667 | Seq. ID No. 2304 |
| 1152b | Seq. ID No. 2305 | 607234 | 607984 | 608667 | Seq. ID No. 2306 |
| 1152c | Seq. ID No. 2307 | 607234 | 607690 | 608667 | Seq. ID No. 2308 |
| 1152d | Seq. ID No. 2309 | 607234 | 607429 | 608667 | Seq. ID No. 2310 |
| 1152e | Seq. ID No. 2311 | 607234 | 607297 | 608667 | Seq. ID No. 2312 |
| 1152f | Seq. ID No. 2313 | 607234 | 607261 | 608667 | Seq. ID No. 2314 |
| 1152g | Seq. ID No. 2315 | 607234 | 607252 | 608667 | Seq. ID No. 2316 |
| 1155a | Seq. ID No. 2317 | 608668 | 609631 | 610128 | Seq. ID No. 2318 |
| 1155b | Seq. ID No. 2319 | 608668 | 609508 | 610128 | Seq. ID No. 2320 |
| 1155c | Seq. ID No. 2321 | 608668 | 609319 | 610128 | Seq. ID No. 2322 |
| 1155d | Seq. ID No. 2323 | 608668 | 609193 | 610128 | Seq. ID No. 2324 |
| 1155e | Seq. ID No. 2325 | 608668 | 608896 | 610128 | Seq. ID No. 2326 |
| 1155f | Seq. ID No. 2327 | 608668 | 608683 | 610128 | Seq. ID No. 2328 |
| 1155g | Seq. ID No. 2329 | 608668 | 608671 | 610128 | Seq. ID No. 2330 |
| 1158a | Seq. ID No. 2331 | 610088 | 610232 | 611965 | Seq. ID No. 2332 |
| 1158b | Seq. ID No. 2333 | 610088 | 610091 | 611965 | Seq. ID No. 2334 |
| 1160 | Seq. ID No. 2335 | 612095 | 612125 | 612553 | Seq. ID No. 2336 |
| 1161a | Seq. ID No. 2337 | 612672 | 612684 | 612935 | Seq. ID No. 2338 |
| 1161b | Seq. ID No. 2339 | 612672 | 612675 | 612935 | Seq. ID No. 2340 |
| 1162 | Seq. ID No. 2341 | 613141 | 613141 | 613064 | Seq. ID No. 2342 |
| 1165 | Seq. ID No. 2343 | 613461 | 613476 | 614456 | Seq. ID No. 2344 |
| 1166 | Seq. ID No. 2345 | 614621 | 614621 | 615358 | Seq. ID No. 2346 |
| 1167a | Seq. ID No. 2347 | 615351 | 615507 | 616433 | Seq. ID No. 2348 |
| 1167b | Seq. ID No. 2349 | 615351 | 615351 | 616433 | Seq. ID No. 2350 |
| 1168 | Seq. ID No. 2351 | 616628 | 616640 | 617020 | Seq. ID No. 2352 |
| 1169a | Seq. ID No. 2353 | 617085 | 618015 | 619262 | Seq. ID No. 2354 |
| 1169b | Seq. ID No. 2355 | 617085 | 617751 | 619262 | Seq. ID No. 2356 |
| 1169c | Seq. ID No. 2357 | 617085 | 617109 | 619262 | Seq. ID No. 2358 |
| 1170 | Seq. ID No. 2359 | 619263 | 619263 | 619499 | Seq. ID No. 2360 |
| 1171a | Seq. ID No. 2361 | 619429 | 619558 | 619929 | Seq. ID No. 2362 |
| 1171c | Seq. ID No. 2363 | 619429 | 619501 | 619929 | Seq. ID No. 2364 |
| 1172 | Seq. ID No. 2365 | 619930 | 619930 | 620520 | Seq. ID No. 2366 |
| 1173 | Seq. ID No. 2367 | 621124 | 621133 | 621411 | Seq. ID No. 2368 |
| 1174 | Seq. ID No. 2369 | 620736 | 620745 | 621947 | Seq. ID No. 2370 |
| 1175 | Seq. ID No. 2371 | 622379 | 622379 | 622125 | Seq. ID No. 2372 |
| 1177 | Seq. ID No. 2373 | 621934 | 621958 | 622941 | Seq. ID No. 2374 |
| 1178 | Seq. ID No. 2375 | 623806 | 623752 | 623462 | Seq. ID No. 2376 |
| 1179a | Seq. ID No. 2377 | 624467 | 624323 | 623553 | Seq. ID No. 2378 |
| 1179b | Seq. ID No. 2379 | 624467 | 624440 | 623553 | Seq. ID No. 2380 |
| 1179c | Seq. ID No. 2381 | 624467 | 624449 | 623553 | Seq. ID No. 2382 |
| 1180a | Seq. ID No. 2383 | 626331 | 625863 | 624772 | Seq. ID No. 2384 |
| 1180b | Seq. ID No. 2385 | 626331 | 626118 | 624772 | Seq. ID No. 2386 |
| 1180c | Seq. ID No. 2387 | 626331 | 626217 | 624772 | Seq. ID No. 2388 |
| 1180d | Seq. ID No. 2389 | 626331 | 626253 | 624772 | Seq. ID No. 2390 |
| 1180e | Seq. ID No. 2391 | 626331 | 626310 | 624772 | Seq. ID No. 2392 |
| 1182a | Seq. ID No. 2393 | 626734 | 626677 | 626441 | Seq. ID No. 2394 |
| 1182b | Seq. ID No. 2395 | 626734 | 626716 | 626441 | Seq. ID No. 2396 |
| 1183a | Seq. ID No. 2397 | 626391 | 626460 | 627632 | Seq. ID No. 2398 |
| 1183b | Seq. ID No. 2399 | 626391 | 626430 | 627632 | Seq. ID No. 2400 |
| 1184 | Seq. ID No. 2401 | 629315 | 629285 | 627993 | Seq. ID No. 2402 |
| 1185a | Seq. ID No. 2403 | 630076 | 629893 | 629300 | Seq. ID No. 2404 |
| 1185b | Seq. ID No. 2405 | 630076 | 630007 | 629300 | Seq. ID No. 2406 |
| 1186a | Seq. ID No. 2407 | 630394 | 630427 | 631026 | Seq. ID No. 2408 |
| 1186b | Seq. ID No. 2409 | 630394 | 630403 | 631026 | Seq. ID No. 2410 |
| 1188a | Seq. ID No. 2411 | 630986 | 631292 | 631714 | Seq. ID No. 2412 |
| 1188b | Seq. ID No. 2413 | 630986 | 631007 | 631714 | Seq. ID No. 2414 |
| 1188c | Seq. ID No. 2415 | 630986 | 630998 | 631714 | Seq. ID No. 2416 |
| 1191 | Seq. ID No. 2417 | 631666 | 631711 | 633153 | Seq. ID No. 2418 |
| 1192a | Seq. ID No. 2419 | 634110 | 634047 | 633199 | Seq. ID No. 2420 |
| 1192b | Seq. ID No. 2421 | 634110 | 634110 | 633199 | Seq. ID No. 2422 |
| 1194 | Seq. ID No. 2423 | 634701 | 634680 | 634123 | Seq. ID No. 2424 |
| 1196a | Seq. ID No. 2425 | 634859 | 634868 | 635863 | Seq. ID No. 2426 |
| 1196b | Seq. ID No. 2427 | 634859 | 634859 | 635863 | Seq. ID No. 2428 |
| 1197 | Seq. ID No. 2429 | 635887 | 635887 | 636759 | Seq. ID No. 2430 |
| 1198 | Seq. ID No. 2431 | 636665 | 636668 | 636898 | Seq. ID No. 2432 |
| 1199a | Seq. ID No. 2433 | 636828 | 636942 | 637436 | Seq. ID No. 2434 |
| 1199b | Seq. ID No. 2435 | 636828 | 636888 | 637436 | Seq. ID No. 2436 |
| 1200 | Seq. ID No. 2437 | 637555 | 637555 | 638352 | Seq. ID No. 2438 |
| 1201 | Seq. ID No. 2439 | 638346 | 638373 | 638648 | Seq. ID No. 2440 |
| 1202a | Seq. ID No. 2441 | 638811 | 638871 | 639152 | Seq. ID No. 2442 |
| 1202b | Seq. ID No. 2443 | 638811 | 638844 | 639152 | Seq. ID No. 2444 |
| 1205 | Seq. ID No. 2445 | 639692 | 639704 | 640546 | Seq. ID No. 2446 |
| 1206 | Seq. ID No. 2447 | 642187 | 642151 | 641477 | Seq. ID No. 2448 |
| 1207 | Seq. ID No. 2449 | 640543 | 640543 | 641538 | Seq. ID No. 2450 |
| 1208 | Seq. ID No. 2451 | 641532 | 641538 | 642890 | Seq. ID No. 2452 |
| 1209 | Seq. ID No. 2453 | 642972 | 642984 | 643718 | Seq. ID No. 2454 |
| 1210a | Seq. ID No. 2455 | 644513 | 644420 | 643800 | Seq. ID No. 2456 |
| 1210b | Seq. ID No. 2457 | 644513 | 644495 | 643800 | Seq. ID No. 2458 |
| 1212 | Seq. ID No. 2459 | 646415 | 646403 | 646137 | Seq. ID No. 2460 |
| 1213a | Seq. ID No. 2461 | 644672 | 644684 | 646174 | Seq. ID No. 2462 |
| 1213b | Seq. ID No. 2463 | 644672 | 644681 | 646174 | Seq. ID No. 2464 |
| 1214 | Seq. ID No. 2465 | 646171 | 646171 | 646944 | Seq. ID No. 2466 |
| 1215a | Seq. ID No. 2467 | 646895 | 647057 | 647500 | Seq. ID No. 2468 |
| 1215b | Seq. ID No. 2469 | 646895 | 646907 | 647500 | Seq. ID No. 2470 |
| 1217c | Seq. ID No. 2471 | 647452 | 647515 | 648060 | Seq. ID No. 2472 |
| 1218a | Seq. ID No. 2473 | 648023 | 648035 | 649042 | Seq. ID No. 2474 |
| 1218b | Seq. ID No. 2475 | 648023 | 648023 | 649042 | Seq. ID No. 2476 |
| 1219 | Seq. ID No. 2477 | 649847 | 649754 | 649371 | Seq. ID No. 2478 |
| 1220 | Seq. ID No. 2479 | 649953 | 649953 | 649813 | Seq. ID No. 2480 |
| 1221a | Seq. ID No. 2481 | 652043 | 652016 | 650058 | Seq. ID No. 2482 |
| 1221b | Seq. ID No. 2483 | 652043 | 652037 | 650058 | Seq. ID No. 2484 |
| 1223 | Seq. ID No. 2485 | 652187 | 652208 | 652873 | Seq. ID No. 2486 |
| 1224a | Seq. ID No. 2487 | 653833 | 653740 | 653129 | Seq. ID No. 2488 |
| 1224b | Seq. ID No. 2489 | 653833 | 653791 | 653129 | Seq. ID No. 2490 |
| 1224c | Seq. ID No. 2491 | 653833 | 653824 | 653129 | Seq. ID No. 2492 |
| 1225 | Seq. ID No. 2493 | 654455 | 654434 | 653970 | Seq. ID No. 2494 |
| 1226 | Seq. ID No. 2495 | 654010 | 654010 | 654294 | Seq. ID No. 2496 |
| 1228a | Seq. ID No. 2497 | 655583 | 655400 | 655020 | Seq. ID No. 2498 |
| 1228b | Seq. ID No. 2499 | 655583 | 655562 | 655020 | Seq. ID No. 2500 |
| 1229b | Seq. ID No. 2501 | 656021 | 655988 | 655689 | Seq. ID No. 2502 |
| 1229c | Seq. ID No. 2503 | 656021 | 655994 | 655689 | Seq. ID No. 2504 |
| 1230 | Seq. ID No. 2505 | 654334 | 654337 | 655968 | Seq. ID No. 2506 |
| 1231 | Seq. ID No. 2507 | 656741 | 656720 | 656064 | Seq. ID No. 2508 |
| 1233a | Seq. ID No. 2509 | 656808 | 656892 | 657533 | Seq. ID No. 2510 |
| 1233b | Seq. ID No. 2511 | 656808 | 656874 | 657533 | Seq. ID No. 2512 |
| 1233c | Seq. ID No. 2513 | 656808 | 656871 | 657533 | Seq. ID No. 2514 |
| 1234a | Seq. ID No. 2515 | 657534 | 657801 | 658301 | Seq. ID No. 2516 |
| 1234b | Seq. ID No. 2517 | 657534 | 657744 | 658301 | Seq. ID No. 2518 |
| 1234c | Seq. ID No. 2519 | 657534 | 657537 | 658301 | Seq. ID No. 2520 |
| 1235 | Seq. ID No. 2521 | 658795 | 658795 | 658484 | Seq. ID No. 2522 |
| 1236a | Seq. ID No. 2523 | 659168 | 659063 | 658815 | Seq. ID No. 2524 |
| 1237 | Seq. ID No. 2525 | 658446 | 658503 | 659438 | Seq. ID No. 2526 |
| 1239 | Seq. ID No. 2527 | 659964 | 659976 | 660188 | Seq. ID No. 2528 |
| 1240 | Seq. ID No. 2529 | 661436 | 661367 | 660249 | Seq. ID No. 2530 |
| 1241a | Seq. ID No. 2531 | 661467 | 662226 | 662597 | Seq. ID No. 2532 |
| 1241b | Seq. ID No. 2533 | 661467 | 661497 | 662597 | Seq. ID No. 2534 |
| 1241c | Seq. ID No. 2535 | 661467 | 661479 | 662597 | Seq. ID No. 2536 |
| 1243 | Seq. ID No. 2537 | 662682 | 662682 | 663986 | Seq. ID No. 2538 |
| 1244 | Seq. ID No. 2539 | 664168 | 664189 | 664956 | Seq. ID No. 2540 |
| 1245a | Seq. ID No. 2541 | 666679 | 665737 | 665099 | Seq. ID No. 2542 |
| 1245b | Seq. ID No. 2543 | 666679 | 666304 | 665099 | Seq. ID No. 2544 |
| 1245c | Seq. ID No. 2545 | 666679 | 666400 | 665099 | Seq. ID No. 2546 |
| 1245d | Seq. ID No. 2547 | 666679 | 666649 | 665099 | Seq. ID No. 2548 |
| 1245e | Seq. ID No. 2549 | 666679 | 666658 | 665099 | Seq. ID No. 2550 |
| 1246a | Seq. ID No. 2551 | 667345 | 667318 | 666701 | Seq. ID No. 2552 |
| 1246b | Seq. ID No. 2553 | 667345 | 667327 | 666701 | Seq. ID No. 2554 |
| 1250a | Seq. ID No. 2555 | 667704 | 668655 | 668966 | Seq. ID No. 2556 |
| 1250b | Seq. ID No. 2557 | 667704 | 667953 | 668966 | Seq. ID No. 2558 |
| 1250c | Seq. ID No. 2559 | 667704 | 667830 | 668966 | Seq. ID No. 2560 |
| 1250d | Seq. ID No. 2561 | 667704 | 667782 | 668966 | Seq. ID No. 2562 |
| 1250e | Seq. ID No. 2563 | 667704 | 667746 | 668966 | Seq. ID No. 2564 |
| 1251a | Seq. ID No. 2565 | 669339 | 669444 | 669770 | Seq. ID No. 2566 |
| 1251b | Seq. ID No. 2567 | 669339 | 669420 | 669770 | Seq. ID No. 2568 |
| 1253a | Seq. ID No. 2569 | 669718 | 670432 | 670668 | Seq. ID No. 2570 |
| 1253b | Seq. ID No. 2571 | 669718 | 670354 | 670668 | Seq. ID No. 2572 |
| 1253c | Seq. ID No. 2573 | 669718 | 669841 | 670668 | Seq. ID No. 2574 |
| 1253d | Seq. ID No. 2575 | 669718 | 669808 | 670668 | Seq. ID No. 2576 |
| 1256 | Seq. ID No. 2577 | 670771 | 670801 | 672036 | Seq. ID No. 2578 |
| 1257a | Seq. ID No. 2579 | 671964 | 672051 | 672518 | Seq. ID No. 2580 |
| 1257b | Seq. ID No. 2581 | 671964 | 672048 | 672518 | Seq. ID No. 2582 |
| 1258a | Seq. ID No. 2583 | 675281 | 674429 | 672591 | Seq. ID No. 2584 |
| 1258b | Seq. ID No. 2585 | 675281 | 675200 | 672591 | Seq. ID No. 2586 |
| 1258c | Seq. ID No. 2587 | 675281 | 675221 | 672591 | Seq. ID No. 2588 |
| 1258d | Seq. ID No. 2589 | 675281 | 675278 | 672591 | Seq. ID No. 2590 |
| 1260 | Seq. ID No. 2591 | 672895 | 672934 | 673179 | Seq. ID No. 2592 |
| 1263 | Seq. ID No. 2593 | 675596 | 675608 | 676525 | Seq. ID No. 2594 |
| 1265 | Seq. ID No. 2595 | 676530 | 676545 | 677219 | Seq. ID No. 2596 |
| 1266a | Seq. ID No. 2597 | 677365 | 677401 | 678045 | Seq. ID No. 2598 |
| 1266b | Seq. ID No. 2599 | 677365 | 677365 | 678045 | Seq. ID No. 2600 |
| 1267a | Seq. ID No. 2601 | 678014 | 678107 | 678361 | Seq. ID No. 2602 |
| 1267b | Seq. ID No. 2603 | 678014 | 678038 | 678361 | Seq. ID No. 2604 |
| 1268a | Seq. ID No. 2605 | 680490 | 680229 | 678862 | Seq. ID No. 2606 |
| 1268b | Seq. ID No. 2607 | 680490 | 680436 | 678862 | Seq. ID No. 2608 |
| 1269 | Seq. ID No. 2609 | 680591 | 680612 | 681463 | Seq. ID No. 2610 |
| 1270 | Seq. ID No. 2611 | 682595 | 682535 | 682245 | Seq. ID No. 2612 |
| 1271 | Seq. ID No. 2613 | 681643 | 681646 | 683115 | Seq. ID No. 2614 |
| 1272a | Seq. ID No. 2615 | 684311 | 684107 | 683223 | Seq. ID No. 2616 |
| 1272b | Seq. ID No. 2617 | 684311 | 684308 | 683223 | Seq. ID No. 2618 |
| 1273 | Seq. ID No. 2619 | 685392 | 685377 | 684289 | Seq. ID No. 2620 |
| 1275a | Seq. ID No. 2621 | 686415 | 686145 | 685393 | Seq. ID No. 2622 |
| 1275b | Seq. ID No. 2623 | 686415 | 686403 | 685393 | Seq. ID No. 2624 |
| 1277 | Seq. ID No. 2625 | 687376 | 687349 | 686396 | Seq. ID No. 2626 |
| 1278a | Seq. ID No. 2627 | 686400 | 686439 | 686732 | Seq. ID No. 2628 |
| 1278b | Seq. ID No. 2629 | 686400 | 686406 | 686732 | Seq. ID No. 2630 |
| 1279 | Seq. ID No. 2631 | 687461 | 687506 | 688435 | Seq. ID No. 2632 |
| 1281 | Seq. ID No. 2633 | 688554 | 688557 | 689963 | Seq. ID No. 2634 |
| 1283a | Seq. ID No. 2635 | 690164 | 690239 | 691681 | Seq. ID No. 2636 |
| 1283b | Seq. ID No. 2637 | 690164 | 690212 | 691681 | Seq. ID No. 2638 |
| 1283c | Seq. ID No. 2639 | 690164 | 690182 | 691681 | Seq. ID No. 2640 |
| 1284 | Seq. ID No. 2641 | 691675 | 691681 | 692520 | Seq. ID No. 2642 |
| 1285a | Seq. ID No. 2643 | 692499 | 692535 | 693674 | Seq. ID No. 2644 |
| 1285b | Seq. ID No. 2645 | 692499 | 692502 | 693674 | Seq. ID No. 2646 |
| 1287a | Seq. ID No. 2647 | 693675 | 693741 | 694412 | Seq. ID No. 2648 |
| 1287b | Seq. ID No. 2649 | 693675 | 693696 | 694412 | Seq. ID No. 2650 |
| 1288 | Seq. ID No. 2651 | 696061 | 696046 | 695036 | Seq. ID No. 2652 |
| 1289 | Seq. ID No. 2653 | 695318 | 695384 | 695629 | Seq. ID No. 2654 |
| 1290 | Seq. ID No. 2655 | 696712 | 696700 | 696062 | Seq. ID No. 2656 |
| 1293a | Seq. ID No. 2657 | 696820 | 697045 | 698178 | Seq. ID No. 2658 |
| 1293b | Seq. ID No. 2659 | 696820 | 696829 | 698178 | Seq. ID No. 2660 |
| 1294a | Seq. ID No. 2661 | 698539 | 698527 | 698231 | Seq. ID No. 2662 |
| 1295 | Seq. ID No. 2663 | 698150 | 698171 | 698863 | Seq. ID No. 2664 |
| 1297a | Seq. ID No. 2665 | 699177 | 699198 | 699776 | Seq. ID No. 2666 |
| 1297b | Seq. ID No. 2667 | 699177 | 699180 | 699776 | Seq. ID No. 2668 |
| 1300 | Seq. ID No. 2669 | 699972 | 699981 | 702449 | Seq. ID No. 2670 |
| 1302 | Seq. ID No. 2671 | 702563 | 702629 | 703615 | Seq. ID No. 2672 |
| 1305a | Seq. ID No. 2673 | 703701 | 703719 | 704396 | Seq. ID No. 2674 |
| 1305b | Seq. ID No. 2675 | 703701 | 703710 | 704396 | Seq. ID No. 2676 |
| 1305c | Seq. ID No. 2677 | 703701 | 703701 | 704396 | Seq. ID No. 2678 |
| 1308a | Seq. ID No. 2679 | 704332 | 704872 | 705273 | Seq. ID No. 2680 |
| 1308b | Seq. ID No. 2681 | 704332 | 704452 | 705273 | Seq. ID No. 2682 |
| 1308c | Seq. ID No. 2683 | 704332 | 704362 | 705273 | Seq. ID No. 2684 |
| 1308d | Seq. ID No. 2685 | 704332 | 704344 | 705273 | Seq. ID No. 2686 |
| 1308e | Seq. ID No. 2687 | 704332 | 704332 | 705273 | Seq. ID No. 2688 |
| 1311 | Seq. ID No. 2689 | 705580 | 705598 | 706314 | Seq. ID No. 2690 |
| 1314b | Seq. ID No. 2691 | 707728 | 707725 | 707432 | Seq. ID No. 2692 |
| 1315 | Seq. ID No. 2693 | 706311 | 706311 | 708119 | Seq. ID No. 2694 |
| 1316 | Seq. ID No. 2695 | 708319 | 708325 | 708573 | Seq. ID No. 2696 |
| 1317 | Seq. ID No. 2697 | 709234 | 709210 | 708947 | Seq. ID No. 2698 |
| 1318 | Seq. ID No. 2699 | 708258 | 708261 | 709274 | Seq. ID No. 2700 |
| 1319a | Seq. ID No. 2701 | 709369 | 709570 | 710250 | Seq. ID No. 2702 |
| 1319b | Seq. ID No. 2703 | 709369 | 709390 | 710250 | Seq. ID No. 2704 |
| 1320a | Seq. ID No. 2705 | 710244 | 710745 | 711068 | Seq. ID No. 2706 |
| 1320b | Seq. ID No. 2707 | 710244 | 710400 | 711068 | Seq. ID No. 2708 |
| 1320c | Seq. ID No. 2709 | 710244 | 710250 | 711068 | Seq. ID No. 2710 |
| 1321 | Seq. ID No. 2711 | 711047 | 711068 | 711586 | Seq. ID No. 2712 |
| 1322a | Seq. ID No. 2713 | 711571 | 711586 | 711831 | Seq. ID No. 2714 |
| 1322b | Seq. ID No. 2715 | 711571 | 711583 | 711831 | Seq. ID No. 2716 |
| 1323b | Seq. ID No. 2717 | 712308 | 712248 | 711997 | Seq. ID No. 2718 |
| 1324 | Seq. ID No. 2719 | 711914 | 711917 | 712600 | Seq. ID No. 2720 |
| 1326a | Seq. ID No. 2721 | 712714 | 712729 | 713361 | Seq. ID No. 2722 |
| 1326b | Seq. ID No. 2723 | 712714 | 712723 | 713361 | Seq. ID No. 2724 |
| 1326c | Seq. ID No. 2725 | 712714 | 712720 | 713361 | Seq. ID No. 2726 |
| 1333 | Seq. ID No. 2727 | 713541 | 713541 | 716393 | Seq. ID No. 2728 |
| 1334a | Seq. ID No. 2729 | 716775 | 716739 | 716479 | Seq. ID No. 2730 |
| 1334b | Seq. ID No. 2731 | 716775 | 716748 | 716479 | Seq. ID No. 2732 |
| 1335a | Seq. ID No. 2733 | 717513 | 717270 | 716776 | Seq. ID No. 2734 |
| 1335b | Seq. ID No. 2735 | 717513 | 717462 | 716776 | Seq. ID No. 2736 |
| 1337 | Seq. ID No. 2737 | 716522 | 716558 | 718135 | Seq. ID No. 2738 |
| 1338 | Seq. ID No. 2739 | 718855 | 718855 | 718586 | Seq. ID No. 2740 |
| 1340a | Seq. ID No. 2741 | 718451 | 718490 | 718822 | Seq. ID No. 2742 |
| 1340b | Seq. ID No. 2743 | 718451 | 718466 | 718822 | Seq. ID No. 2744 |
| 1341a | Seq. ID No. 2745 | 718816 | 718858 | 719163 | Seq. ID No. 2746 |
| 1341c | Seq. ID No. 2747 | 718816 | 718822 | 719163 | Seq. ID No. 2748 |
| 1344a | Seq. ID No. 2749 | 719333 | 719396 | 720334 | Seq. ID No. 2750 |
| 1344b | Seq. ID No. 2751 | 719333 | 719381 | 720334 | Seq. ID No. 2752 |
| 1348 | Seq. ID No. 2753 | 720382 | 720400 | 721263 | Seq. ID No. 2754 |
| 1349a | Seq. ID No. 2755 | 721264 | 721312 | 722295 | Seq. ID No. 2756 |
| 1349b | Seq. ID No. 2757 | 721264 | 721276 | 722295 | Seq. ID No. 2758 |
| 1349c | Seq. ID No. 2759 | 721264 | 721267 | 722295 | Seq. ID No. 2760 |
| 1350a | Seq. ID No. 2761 | 722342 | 722369 | 722797 | Seq. ID No. 2762 |
| 1350b | Seq. ID No. 2763 | 722342 | 722348 | 722797 | Seq. ID No. 2764 |
| 1353 | Seq. ID No. 2765 | 722851 | 722869 | 723843 | Seq. ID No. 2766 |
| 1355 | Seq. ID No. 2767 | 723944 | 723944 | 725668 | Seq. ID No. 2768 |
| 1358a | Seq. ID No. 2769 | 725732 | 725753 | 726643 | Seq. ID No. 2770 |
| 1358b | Seq. ID No. 2771 | 725732 | 725732 | 726643 | Seq. ID No. 2772 |
| 1359 | Seq. ID No. 2773 | 726616 | 726640 | 727635 | Seq. ID No. 2774 |
| 1361 | Seq. ID No. 2775 | 727639 | 727651 | 728580 | Seq. ID No. 2776 |
| 1364a | Seq. ID No. 2777 | 730140 | 730065 | 729379 | Seq. ID No. 2778 |
| 1364b | Seq. ID No. 2779 | 730140 | 730092 | 729379 | Seq. ID No. 2780 |
| 1364c | Seq. ID No. 2781 | 730140 | 730107 | 729379 | Seq. ID No. 2782 |
| 1365b | Seq. ID No. 2783 | 729761 | 729827 | 730129 | Seq. ID No. 2784 |
| 1366a | Seq. ID No. 2785 | 730210 | 730483 | 730836 | Seq. ID No. 2786 |
| 1366b | Seq. ID No. 2787 | 730210 | 730243 | 730836 | Seq. ID No. 2788 |
| 1367 | Seq. ID No. 2789 | 730970 | 730988 | 732001 | Seq. ID No. 2790 |
| 1368 | Seq. ID No. 2791 | 732002 | 732038 | 733033 | Seq. ID No. 2792 |
| 1371 | Seq. ID No. 2793 | 733169 | 733175 | 734359 | Seq. ID No. 2794 |
| 1373a | Seq. ID No. 2795 | 734383 | 734389 | 735144 | Seq. ID No. 2796 |
| 1373b | Seq. ID No. 2797 | 734383 | 734386 | 735144 | Seq. ID No. 2798 |
| 1374 | Seq. ID No. 2799 | 735111 | 735144 | 736679 | Seq. ID No. 2800 |
| 1375 | Seq. ID No. 2801 | 737472 | 737457 | 737191 | Seq. ID No. 2802 |
| 1377 | Seq. ID No. 2803 | 736676 | 736730 | 738028 | Seq. ID No. 2804 |
| 1378 | Seq. ID No. 2805 | 738364 | 738346 | 738119 | Seq. ID No. 2806 |
| 1379 | Seq. ID No. 2807 | 738645 | 738612 | 738340 | Seq. ID No. 2808 |
| 1380 | Seq. ID No. 2809 | 738987 | 738954 | 738646 | Seq. ID No. 2810 |
| 1381a | Seq. ID No. 2811 | 738197 | 738560 | 739288 | Seq. ID No. 2812 |
| 1381b | Seq. ID No. 2813 | 738197 | 738221 | 739288 | Seq. ID No. 2814 |
| 1382 | Seq. ID No. 2815 | 739289 | 739298 | 739879 | Seq. ID No. 2816 |
| 1383 | Seq. ID No. 2817 | 739834 | 739906 | 740064 | Seq. ID No. 2818 |
| 1384a | Seq. ID No. 2819 | 740592 | 740637 | 740843 | Seq. ID No. 2820 |
| 1384b | Seq. ID No. 2821 | 740592 | 740613 | 740843 | Seq. ID No. 2822 |
| 1384c | Seq. ID No. 2823 | 740592 | 740607 | 740843 | Seq. ID No. 2824 |
| 1386a | Seq. ID No. 2825 | 740904 | 740955 | 741692 | Seq. ID No. 2826 |
| 1386b | Seq. ID No. 2827 | 740904 | 740949 | 741692 | Seq. ID No. 2828 |
| 1387a | Seq. ID No. 2829 | 742161 | 741981 | 741694 | Seq. ID No. 2830 |
| 1388b | Seq. ID No. 2831 | 741439 | 741448 | 741723 | Seq. ID No. 2832 |
| 1389a | Seq. ID No. 2833 | 742245 | 742329 | 742586 | Seq. ID No. 2834 |
| 1393a | Seq. ID No. 2835 | 744190 | 744004 | 743774 | Seq. ID No. 2836 |
| 1393b | Seq. ID No. 2837 | 744190 | 744088 | 743774 | Seq. ID No. 2838 |
| 1393c | Seq. ID No. 2839 | 744190 | 744118 | 743774 | Seq. ID No. 2840 |
| 1393d | Seq. ID No. 2841 | 744190 | 744172 | 743774 | Seq. ID No. 2842 |
| 1394 | Seq. ID No. 2843 | 741716 | 741716 | 744145 | Seq. ID No. 2844 |
| 1395a | Seq. ID No. 2845 | 743835 | 743886 | 744179 | Seq. ID No. 2846 |
| 1396 | Seq. ID No. 2847 | 744166 | 744166 | 744624 | Seq. ID No. 2848 |
| 1397 | Seq. ID No. 2849 | 745009 | 745003 | 744779 | Seq. ID No. 2850 |
| 1398 | Seq. ID No. 2851 | 745395 | 745395 | 745583 | Seq. ID No. 2852 |
| 1399a | Seq. ID No. 2853 | 746205 | 746151 | 745810 | Seq. ID No. 2854 |
| 1399b | Seq. ID No. 2855 | 746205 | 746178 | 745810 | Seq. ID No. 2856 |
| 1400 | Seq. ID No. 2857 | 745613 | 745613 | 745813 | Seq. ID No. 2858 |
| 1401 | Seq. ID No. 2859 | 745943 | 745943 | 746104 | Seq. ID No. 2860 |
| 1402c | Seq. ID No. 2861 | 746612 | 746558 | 746190 | Seq. ID No. 2862 |
| 1405 | Seq. ID No. 2863 | 746924 | 746924 | 749635 | Seq. ID No. 2864 |
| 1407b | Seq. ID No. 2865 | 751061 | 751055 | 750588 | Seq. ID No. 2866 |
| 1408 | Seq. ID No. 2867 | 749626 | 749647 | 751767 | Seq. ID No. 2868 |
| 1409 | Seq. ID No. 2869 | 751768 | 751768 | 754359 | Seq. ID No. 2870 |
| 1410 | Seq. ID No. 2871 | 754341 | 754374 | 755108 | Seq. ID No. 2872 |
| 1411a | Seq. ID No. 2873 | 755080 | 755485 | 755841 | Seq. ID No. 2874 |
| 1411b | Seq. ID No. 2875 | 755080 | 755344 | 755841 | Seq. ID No. 2876 |
| 1411c | Seq. ID No. 2877 | 755080 | 755092 | 755841 | Seq. ID No. 2878 |
| 1412a | Seq. ID No. 2879 | 757589 | 757421 | 756222 | Seq. ID No. 2880 |
| 1412b | Seq. ID No. 2881 | 757589 | 757586 | 756222 | Seq. ID No. 2882 |
| 1414a | Seq. ID No. 2883 | 756988 | 757285 | 757674 | Seq. ID No. 2884 |
| 1414b | Seq. ID No. 2885 | 756988 | 757180 | 757674 | Seq. ID No. 2886 |
| 1414c | Seq. ID No. 2887 | 756988 | 757003 | 757674 | Seq. ID No. 2888 |
| 1415 | Seq. ID No. 2889 | 758110 | 758110 | 758241 | Seq. ID No. 2890 |
| 1416 | Seq. ID No. 2891 | 759768 | 759735 | 758569 | Seq. ID No. 2892 |
| 1418 | Seq. ID No. 2893 | 758771 | 758771 | 759010 | Seq. ID No. 2894 |
| 1419 | Seq. ID No. 2895 | 760783 | 760735 | 760511 | Seq. ID No. 2896 |
| 1420a | Seq. ID No. 2897 | 760029 | 760194 | 760772 | Seq. ID No. 2898 |
| 1420b | Seq. ID No. 2899 | 760029 | 760029 | 760772 | Seq. ID No. 2900 |
| 1422 | Seq. ID No. 2901 | 760703 | 760775 | 762157 | Seq. ID No. 2902 |
| 1423 | Seq. ID No. 2903 | 762262 | 762283 | 762516 | Seq. ID No. 2904 |
| 1426a | Seq. ID No. 2905 | 762510 | 762915 | 763571 | Seq. ID No. 2906 |
| 1426b | Seq. ID No. 2907 | 762510 | 762528 | 763571 | Seq. ID No. 2908 |
| 1427a | Seq. ID No. 2909 | 763552 | 763810 | 763941 | Seq. ID No. 2910 |
| 1427b | Seq. ID No. 2911 | 763552 | 763564 | 763941 | Seq. ID No. 2912 |
| 1428 | Seq. ID No. 2913 | 763895 | 763949 | 764311 | Seq. ID No. 2914 |
| 1429a | Seq. ID No. 2915 | 765122 | 765107 | 764379 | Seq. ID No. 2916 |
| 1429b | Seq. ID No. 2917 | 765122 | 765116 | 764379 | Seq. ID No. 2918 |
| 1431 | Seq. ID No. 2919 | 765350 | 765365 | 766138 | Seq. ID No. 2920 |
| 1432 | Seq. ID No. 2921 | 766139 | 766154 | 767452 | Seq. ID No. 2922 |
| 1433 | Seq. ID No. 2923 | 767422 | 767449 | 768687 | Seq. ID No. 2924 |
| 1434a | Seq. ID No. 2925 | 768641 | 768761 | 769153 | Seq. ID No. 2926 |
| 1434b | Seq. ID No. 2927 | 768641 | 768674 | 769153 | Seq. ID No. 2928 |
| 1437a | Seq. ID No. 2929 | 769101 | 769194 | 770588 | Seq. ID No. 2930 |
| 1437b | Seq. ID No. 2931 | 769101 | 769185 | 770588 | Seq. ID No. 2932 |
| 1437c | Seq. ID No. 2933 | 769101 | 769119 | 770588 | Seq. ID No. 2934 |
| 1438 | Seq. ID No. 2935 | 771081 | 771093 | 771485 | Seq. ID No. 2936 |
| 1439a | Seq. ID No. 2937 | 771556 | 772081 | 772368 | Seq. ID No. 2938 |
| 1439b | Seq. ID No. 2939 | 771556 | 771832 | 772368 | Seq. ID No. 2940 |
| 1439c | Seq. ID No. 2941 | 771556 | 771688 | 772368 | Seq. ID No. 2942 |
| 1439d | Seq. ID No. 2943 | 771556 | 771565 | 772368 | Seq. ID No. 2944 |
| 1439e | Seq. ID No. 2945 | 771556 | 771556 | 772368 | Seq. ID No. 2946 |
| 1440a | Seq. ID No. 2947 | 772260 | 773019 | 773900 | Seq. ID No. 2948 |
| 1440b | Seq. ID No. 2949 | 772260 | 772659 | 773900 | Seq. ID No. 2950 |
| 1440c | Seq. ID No. 2951 | 772260 | 772371 | 773900 | Seq. ID No. 2952 |
| 1440d | Seq. ID No. 2953 | 772260 | 772347 | 773900 | Seq. ID No. 2954 |
| 1442a | Seq. ID No. 2955 | 773837 | 774053 | 774535 | Seq. ID No. 2956 |
| 1442b | Seq. ID No. 2957 | 773837 | 773897 | 774535 | Seq. ID No. 2958 |
| 1445a | Seq. ID No. 2959 | 774811 | 775252 | 775797 | Seq. ID No. 2960 |
| 1445b | Seq. ID No. 2961 | 774811 | 775042 | 775797 | Seq. ID No. 2962 |
| 1445c | Seq. ID No. 2963 | 774811 | 774853 | 775797 | Seq. ID No. 2964 |
| 1446a | Seq. ID No. 2965 | 776293 | 776224 | 775967 | Seq. ID No. 2966 |
| 1446b | Seq. ID No. 2967 | 776293 | 776278 | 775967 | Seq. ID No. 2968 |
| 1447a | Seq. ID No. 2969 | 776362 | 776419 | 777786 | Seq. ID No. 2970 |
| 1447b | Seq. ID No. 2971 | 776362 | 776377 | 777786 | Seq. ID No. 2972 |
| 1448 | Seq. ID No. 2973 | 778141 | 778195 | 778794 | Seq. ID No. 2974 |
| 1449a | Seq. ID No. 2975 | 778847 | 778862 | 779716 | Seq. ID No. 2976 |
| 1449b | Seq. ID No. 2977 | 778847 | 778853 | 779716 | Seq. ID No. 2978 |
| 1451 | Seq. ID No. 2979 | 779700 | 779706 | 780320 | Seq. ID No. 2980 |
| 1452a | Seq. ID No. 2981 | 780286 | 780310 | 781452 | Seq. ID No. 2982 |
| 1452b | Seq. ID No. 2983 | 780286 | 780295 | 781452 | Seq. ID No. 2984 |
| 1453 | Seq. ID No. 2985 | 781774 | 781777 | 782313 | Seq. ID No. 2986 |
| 1454 | Seq. ID No. 2987 | 782603 | 782594 | 782319 | Seq. ID No. 2988 |
| 1455a | Seq. ID No. 2989 | 782323 | 782416 | 782772 | Seq. ID No. 2990 |
| 1455b | Seq. ID No. 2991 | 782323 | 782359 | 782772 | Seq. ID No. 2992 |
| 1456 | Seq. ID No. 2993 | 783394 | 783373 | 782858 | Seq. ID No. 2994 |
| 1457a | Seq. ID No. 2995 | 785272 | 784672 | 783587 | Seq. ID No. 2996 |
| 1457b | Seq. ID No. 2997 | 785272 | 784795 | 783587 | Seq. ID No. 2998 |
| 1457c | Seq. ID No. 2999 | 785272 | 785272 | 783587 | Seq. ID No. 3000 |
| 1459 | Seq. ID No. 3001 | 785453 | 785486 | 785917 | Seq. ID No. 3002 |
| 1462a | Seq. ID No. 3003 | 785979 | 786078 | 787760 | Seq. ID No. 3004 |
| 1462b | Seq. ID No. 3005 | 785979 | 785994 | 787760 | Seq. ID No. 3006 |
| 1463a | Seq. ID No. 3007 | 787735 | 787813 | 788136 | Seq. ID No. 3008 |
| 1463b | Seq. ID No. 3009 | 787735 | 787753 | 788136 | Seq. ID No. 3010 |
| 1463c | Seq. ID No. 3011 | 787735 | 787747 | 788136 | Seq. ID No. 3012 |
| 1466a | Seq. ID No. 3013 | 788108 | 788501 | 789526 | Seq. ID No. 3014 |
| 1466b | Seq. ID No. 3015 | 788108 | 788249 | 789526 | Seq. ID No. 3016 |
| 1466c | Seq. ID No. 3017 | 788108 | 788147 | 789526 | Seq. ID No. 3018 |
| 1466d | Seq. ID No. 3019 | 788108 | 788120 | 789526 | Seq. ID No. 3020 |
| 1467 | Seq. ID No. 3021 | 790286 | 790256 | 789855 | Seq. ID No. 3022 |
| 1469a | Seq. ID No. 3023 | 789757 | 790642 | 791115 | Seq. ID No. 3024 |
| 1469b | Seq. ID No. 3025 | 789757 | 790531 | 791115 | Seq. ID No. 3026 |
| 1469c | Seq. ID No. 3027 | 789757 | 789769 | 791115 | Seq. ID No. 3028 |
| 1470a | Seq. ID No. 3029 | 792128 | 791798 | 791205 | Seq. ID No. 3030 |
| 1470b | Seq. ID No. 3031 | 792128 | 792008 | 791205 | Seq. ID No. 3032 |
| 1470c | Seq. ID No. 3033 | 792128 | 792116 | 791205 | Seq. ID No. 3034 |
| 1471 | Seq. ID No. 3035 | 792254 | 792257 | 793039 | Seq. ID No. 3036 |
| 1472b | Seq. ID No. 3037 | 793681 | 793612 | 793046 | Seq. ID No. 3038 |
| 1472c | Seq. ID No. 3039 | 793681 | 793666 | 793046 | Seq. ID No. 3040 |
| 1474 | Seq. ID No. 3041 | 793841 | 793841 | 794050 | Seq. ID No. 3042 |
| 1475 | Seq. ID No. 3043 | 794342 | 794369 | 795070 | Seq. ID No. 3044 |
| 1476 | Seq. ID No. 3045 | 795230 | 795236 | 795805 | Seq. ID No. 3046 |
| 1477a | Seq. ID No. 3047 | 796616 | 796430 | 795831 | Seq. ID No. 3048 |
| 1477b | Seq. ID No. 3049 | 796616 | 796589 | 795831 | Seq. ID No. 3050 |
| 1479a | Seq. ID No. 3051 | 796810 | 797002 | 798135 | Seq. ID No. 3052 |
| 1479b | Seq. ID No. 3053 | 796810 | 796867 | 798135 | Seq. ID No. 3054 |
| 1479c | Seq. ID No. 3055 | 796810 | 796813 | 798135 | Seq. ID No. 3056 |
| 1482a | Seq. ID No. 3057 | 798771 | 798831 | 799577 | Seq. ID No. 3058 |
| 1482b | Seq. ID No. 3059 | 798771 | 798774 | 799577 | Seq. ID No. 3060 |
| 1483 | Seq. ID No. 3061 | 799724 | 799736 | 800257 | Seq. ID No. 3062 |
| 1484a | Seq. ID No. 3063 | 800458 | 801334 | 801843 | Seq. ID No. 3064 |
| 1484b | Seq. ID No. 3065 | 800458 | 800473 | 801843 | Seq. ID No. 3066 |
| 1484c | Seq. ID No. 3067 | 800458 | 800458 | 801843 | Seq. ID No. 3068 |
| 1486 | Seq. ID No. 3069 | 801844 | 801853 | 802452 | Seq. ID No. 3070 |
| 1487 | Seq. ID No. 3071 | 802771 | 802768 | 802532 | Seq. ID No. 3072 |
| 1488a | Seq. ID No. 3073 | 802224 | 802977 | 803219 | Seq. ID No. 3074 |
| 1488b | Seq. ID No. 3075 | 802224 | 802917 | 803219 | Seq. ID No. 3076 |
| 1488c | Seq. ID No. 3077 | 802224 | 802788 | 803219 | Seq. ID No. 3078 |
| 1488d | Seq. ID No. 3079 | 802224 | 802464 | 803219 | Seq. ID No. 3080 |
| 1488e | Seq. ID No. 3081 | 802224 | 802431 | 803219 | Seq. ID No. 3082 |
| 1488f | Seq. ID No. 3083 | 802224 | 802377 | 803219 | Seq. ID No. 3084 |
| 1488g | Seq. ID No. 3085 | 802224 | 802323 | 803219 | Seq. ID No. 3086 |
| 1489 | Seq. ID No. 3087 | 803568 | 803550 | 803326 | Seq. ID No. 3088 |
| 1490 | Seq. ID No. 3089 | 803135 | 803330 | 803674 | Seq. ID No. 3090 |
| 1494 | Seq. ID No. 3091 | 803890 | 803929 | 805041 | Seq. ID No. 3092 |
| 1497 | Seq. ID No. 3093 | 805042 | 805048 | 806508 | Seq. ID No. 3094 |
| 1498a | Seq. ID No. 3095 | 806363 | 806543 | 807457 | Seq. ID No. 3096 |
| 1498b | Seq. ID No. 3097 | 806363 | 806423 | 807457 | Seq. ID No. 3098 |
| 1498c | Seq. ID No. 3099 | 806363 | 806396 | 807457 | Seq. ID No. 3100 |
| 1498d | Seq. ID No. 3101 | 806363 | 806393 | 807457 | Seq. ID No. 3102 |
| 1499 | Seq. ID No. 3103 | 809397 | 809382 | 808153 | Seq. ID No. 3104 |
| 1502a | Seq. ID No. 3105 | 809502 | 809565 | 809828 | Seq. ID No. 3106 |
| 1502b | Seq. ID No. 3107 | 809502 | 809502 | 809828 | Seq. ID No. 3108 |
| 1503a | Seq. ID No. 3109 | 809829 | 809847 | 810431 | Seq. ID No. 3110 |
| 1503b | Seq. ID No. 3111 | 809829 | 809835 | 810431 | Seq. ID No. 3112 |
| 1506a | Seq. ID No. 3113 | 810510 | 810666 | 812906 | Seq. ID No. 3114 |
| 1506b | Seq. ID No. 3115 | 810510 | 810621 | 812906 | Seq. ID No. 3116 |
| 1506c | Seq. ID No. 3117 | 810510 | 810525 | 812906 | Seq. ID No. 3118 |
| 1507a | Seq. ID No. 3119 | 813291 | 813231 | 812944 | Seq. ID No. 3120 |
| 1507b | Seq. ID No. 3121 | 813291 | 813246 | 812944 | Seq. ID No. 3122 |
| 1507c | Seq. ID No. 3123 | 813291 | 813282 | 812944 | Seq. ID No. 3124 |
| 1508 | Seq. ID No. 3125 | 812996 | 813032 | 813340 | Seq. ID No. 3126 |
| 1510a | Seq. ID No. 3127 | 813521 | 813737 | 815023 | Seq. ID No. 3128 |
| 1510b | Seq. ID No. 3129 | 813521 | 813704 | 815023 | Seq. ID No. 3130 |
| 1510c | Seq. ID No. 3131 | 813521 | 813545 | 815023 | Seq. ID No. 3132 |
| 1510d | Seq. ID No. 3133 | 813521 | 813524 | 815023 | Seq. ID No. 3134 |
| 1511 | Seq. ID No. 3135 | 815078 | 815084 | 815920 | Seq. ID No. 3136 |
| 1512 | Seq. ID No. 3137 | 815892 | 815910 | 816524 | Seq. ID No. 3138 |
| 1513a | Seq. ID No. 3139 | 816525 | 816570 | 817034 | Seq. ID No. 3140 |
| 1513b | Seq. ID No. 3141 | 816525 | 816525 | 817034 | Seq. ID No. 3142 |
| 1516 | Seq. ID No. 3143 | 817055 | 817070 | 817537 | Seq. ID No. 3144 |
| 1517a | Seq. ID No. 3145 | 818704 | 818314 | 817709 | Seq. ID No. 3146 |
| 1517b | Seq. ID No. 3147 | 818704 | 818608 | 817709 | Seq. ID No. 3148 |
| 1517c | Seq. ID No. 3149 | 818704 | 818614 | 817709 | Seq. ID No. 3150 |
| 1518a | Seq. ID No. 3151 | 818067 | 818139 | 818636 | Seq. ID No. 3152 |
| 1518b | Seq. ID No. 3153 | 818067 | 818085 | 818636 | Seq. ID No. 3154 |
| 1520 | Seq. ID No. 3155 | 818738 | 818753 | 819112 | Seq. ID No. 3156 |
| 1522 | Seq. ID No. 3157 | 819117 | 819150 | 819596 | Seq. ID No. 3158 |
| 1523 | Seq. ID No. 3159 | 820780 | 820756 | 819647 | Seq. ID No. 3160 |
| 1528 | Seq. ID No. 3161 | 820968 | 820968 | 821969 | Seq. ID No. 3162 |
| 1530a | Seq. ID No. 3163 | 824730 | 824625 | 822091 | Seq. ID No. 3164 |
| 1530b | Seq. ID No. 3165 | 824730 | 824706 | 822091 | Seq. ID No. 3166 |
| 1533a | Seq. ID No. 3167 | 825870 | 825864 | 825565 | Seq. ID No. 3168 |
| 1534a | Seq. ID No. 3169 | 824975 | 825119 | 826375 | Seq. ID No. 3170 |
| 1534b | Seq. ID No. 3171 | 824975 | 824981 | 826375 | Seq. ID No. 3172 |
| 1535a | Seq. ID No. 3173 | 826056 | 826164 | 826391 | Seq. ID No. 3174 |
| 1536a | Seq. ID No. 3175 | 826336 | 826537 | 826698 | Seq. ID No. 3176 |
| 1536b | Seq. ID No. 3177 | 826336 | 826345 | 826698 | Seq. ID No. 3178 |
| 1537 | Seq. ID No. 3179 | 827893 | 827875 | 826907 | Seq. ID No. 3180 |
| 1542 | Seq. ID No. 3181 | 829139 | 829166 | 829660 | Seq. ID No. 3182 |
| 1543 | Seq. ID No. 3183 | 828142 | 828145 | 829974 | Seq. ID No. 3184 |
| 1544 | Seq. ID No. 3185 | 832325 | 832298 | 830493 | Seq. ID No. 3186 |
| 1547a | Seq. ID No. 3187 | 832494 | 832638 | 833225 | Seq. ID No. 3188 |
| 1547b | Seq. ID No. 3189 | 832494 | 832497 | 833225 | Seq. ID No. 3190 |
| 1548 | Seq. ID No. 3191 | 834559 | 834523 | 833408 | Seq. ID No. 3192 |
| 1552 | Seq. ID No. 3193 | 836319 | 836316 | 836029 | Seq. ID No. 3194 |
| 1553a | Seq. ID No. 3195 | 835285 | 835321 | 836385 | Seq. ID No. 3196 |
| 1553b | Seq. ID No. 3197 | 835285 | 835303 | 836385 | Seq. ID No. 3198 |
| 1554a | Seq. ID No. 3199 | 836324 | 837011 | 837400 | Seq. ID No. 3200 |
| 1554b | Seq. ID No. 3201 | 836324 | 836807 | 837400 | Seq. ID No. 3202 |
| 1554c | Seq. ID No. 3203 | 836324 | 836333 | 837400 | Seq. ID No. 3204 |
| 1555 | Seq. ID No. 3205 | 837622 | 837649 | 839016 | Seq. ID No. 3206 |
| 1556a | Seq. ID No. 3207 | 839851 | 840013 | 840648 | Seq. ID No. 3208 |
| 1556b | Seq. ID No. 3209 | 839851 | 839893 | 840648 | Seq. ID No. 3210 |
| 1556c | Seq. ID No. 3211 | 839851 | 839860 | 840648 | Seq. ID No. 3212 |
| 1557a | Seq. ID No. 3213 | 840617 | 840911 | 841375 | Seq. ID No. 3214 |
| 1557b | Seq. ID No. 3215 | 840617 | 840770 | 841375 | Seq. ID No. 3216 |
| 1557c | Seq. ID No. 3217 | 840617 | 840632 | 841375 | Seq. ID No. 3218 |
| 1559a | Seq. ID No. 3219 | 841324 | 841705 | 842100 | Seq. ID No. 3220 |
| 1559b | Seq. ID No. 3221 | 841324 | 841372 | 842100 | Seq. ID No. 3222 |
| 1563 | Seq. ID No. 3223 | 843403 | 843385 | 843149 | Seq. ID No. 3224 |
| 1564a | Seq. ID No. 3225 | 842064 | 842112 | 843983 | Seq. ID No. 3226 |
| 1564b | Seq. ID No. 3227 | 842064 | 842106 | 843983 | Seq. ID No. 3228 |
| 1565 | Seq. ID No. 3229 | 845451 | 845418 | 844252 | Seq. ID No. 3230 |
| 1567 | Seq. ID No. 3231 | 845601 | 845766 | 845942 | Seq. ID No. 3232 |
| 1568b | Seq. ID No. 3233 | 845896 | 845899 | 846222 | Seq. ID No. 3234 |
| 1569 | Seq. ID No. 3235 | 846223 | 846256 | 846519 | Seq. ID No. 3236 |
| 1570 | Seq. ID No. 3237 | 846792 | 846792 | 846953 | Seq. ID No. 3238 |
| 1571 | Seq. ID No. 3239 | 847452 | 847467 | 848087 | Seq. ID No. 3240 |
| 1572a | Seq. ID No. 3241 | 848056 | 849394 | 849630 | Seq. ID No. 3242 |
| 1572b | Seq. ID No. 3243 | 848056 | 848599 | 849630 | Seq. ID No. 3244 |
| 1572c | Seq. ID No. 3245 | 848056 | 848353 | 849630 | Seq. ID No. 3246 |
| 1572d | Seq. ID No. 3247 | 848056 | 848200 | 849630 | Seq. ID No. 3248 |
| 1572e | Seq. ID No. 3249 | 848056 | 848065 | 849630 | Seq. ID No. 3250 |
| 1573 | Seq. ID No. 3251 | 849498 | 849618 | 850292 | Seq. ID No. 3252 |
| 1576 | Seq. ID No. 3253 | 850427 | 850442 | 851671 | Seq. ID No. 3254 |
| 1577a | Seq. ID No. 3255 | 852156 | 852168 | 852893 | Seq. ID No. 3256 |
| 1577b | Seq. ID No. 3257 | 852156 | 852165 | 852893 | Seq. ID No. 3258 |
| 1578 | Seq. ID No. 3259 | 852839 | 852860 | 853744 | Seq. ID No. 3260 |
| 1580a | Seq. ID No. 3261 | 854091 | 854103 | 854405 | Seq. ID No. 3262 |
| 1580b | Seq. ID No. 3263 | 854091 | 854091 | 854405 | Seq. ID No. 3264 |
| 1581 | Seq. ID No. 3265 | 854392 | 854407 | 854871 | Seq. ID No. 3266 |
| 1582a | Seq. ID No. 3267 | 854822 | 854933 | 855163 | Seq. ID No. 3268 |
| 1582b | Seq. ID No. 3269 | 854822 | 854855 | 855163 | Seq. ID No. 3270 |
| 1582c | Seq. ID No. 3271 | 854822 | 854837 | 855163 | Seq. ID No. 3272 |
| 1584 | Seq. ID No. 3273 | 855136 | 855160 | 855618 | Seq. ID No. 3274 |
| 1585a | Seq. ID No. 3275 | 855593 | 855626 | 856216 | Seq. ID No. 3276 |
| 1585b | Seq. ID No. 3277 | 855593 | 855602 | 856216 | Seq. ID No. 3278 |
| 1587a | Seq. ID No. 3279 | 856349 | 856619 | 857485 | Seq. ID No. 3280 |
| 1587b | Seq. ID No. 3281 | 856349 | 856424 | 857485 | Seq. ID No. 3282 |
| 1587c | Seq. ID No. 3283 | 856349 | 856373 | 857485 | Seq. ID No. 3284 |
| 1588 | Seq. ID No. 3285 | 857486 | 857504 | 858061 | Seq. ID No. 3286 |
| 1590 | Seq. ID No. 3287 | 858037 | 858088 | 858495 | Seq. ID No. 3288 |
| 1591 | Seq. ID No. 3289 | 858485 | 858488 | 858988 | Seq. ID No. 3290 |
| 1592a | Seq. ID No. 3291 | 859040 | 859280 | 860416 | Seq. ID No. 3292 |
| 1592b | Seq. ID No. 3293 | 859040 | 859052 | 860416 | Seq. ID No. 3294 |
| 1593 | Seq. ID No. 3295 | 860376 | 860406 | 860675 | Seq. ID No. 3296 |
| 1594a | Seq. ID No. 3297 | 860676 | 860784 | 861578 | Seq. ID No. 3298 |
| 1594b | Seq. ID No. 3299 | 860676 | 860685 | 861578 | Seq. ID No. 3300 |
| 1595a | Seq. ID No. 3301 | 861559 | 861571 | 862377 | Seq. ID No. 3302 |
| 1595b | Seq. ID No. 3303 | 861559 | 861562 | 862377 | Seq. ID No. 3304 |
| 1598a | Seq. ID No. 3305 | 862517 | 862631 | 864310 | Seq. ID No. 3306 |
| 1598b | Seq. ID No. 3307 | 862517 | 862601 | 864310 | Seq. ID No. 3308 |
| 1600a | Seq. ID No. 3309 | 864687 | 864807 | 866219 | Seq. ID No. 3310 |
| 1600b | Seq. ID No. 3311 | 864687 | 864798 | 866219 | Seq. ID No. 3312 |
| 1600c | Seq. ID No. 3313 | 864687 | 864702 | 866219 | Seq. ID No. 3314 |
| 1602 | Seq. ID No. 3315 | 866403 | 866418 | 867107 | Seq. ID No. 3316 |
| 1606a | Seq. ID No. 3317 | 867044 | 867632 | 868624 | Seq. ID No. 3318 |
| 1606b | Seq. ID No. 3319 | 867044 | 867104 | 868624 | Seq. ID No. 3320 |
| 1607a | Seq. ID No. 3321 | 869654 | 869564 | 868668 | Seq. ID No. 3322 |
| 1607b | Seq. ID No. 3323 | 869654 | 869636 | 868668 | Seq. ID No. 3324 |
| 1608 | Seq. ID No. 3325 | 868639 | 868657 | 868893 | Seq. ID No. 3326 |
| 1610a | Seq. ID No. 3327 | 870103 | 870085 | 869714 | Seq. ID No. 3328 |
| 1610b | Seq. ID No. 3329 | 870103 | 870094 | 869714 | Seq. ID No. 3330 |
| 1612 | Seq. ID No. 3331 | 871769 | 871679 | 871356 | Seq. ID No. 3332 |
| 1616a | Seq. ID No. 3333 | 870406 | 870709 | 872826 | Seq. ID No. 3334 |
| 1616b | Seq. ID No. 3335 | 870406 | 870418 | 872826 | Seq. ID No. 3336 |
| 1619 | Seq. ID No. 3337 | 873157 | 873166 | 874266 | Seq. ID No. 3338 |
| 1621 | Seq. ID No. 3339 | 875446 | 875428 | 875066 | Seq. ID No. 3340 |
| 1622a | Seq. ID No. 3341 | 875866 | 875788 | 875537 | Seq. ID No. 3342 |
| 1623a | Seq. ID No. 3343 | 874398 | 874665 | 875993 | Seq. ID No. 3344 |
| 1623b | Seq. ID No. 3345 | 874398 | 874437 | 875993 | Seq. ID No. 3346 |
| 1623c | Seq. ID No. 3347 | 874398 | 874410 | 875993 | Seq. ID No. 3348 |
| 1624 | Seq. ID No. 3349 | 876797 | 876749 | 876447 | Seq. ID No. 3350 |
| 1625a | Seq. ID No. 3351 | 875908 | 876511 | 877038 | Seq. ID No. 3352 |
| 1625b | Seq. ID No. 3353 | 875908 | 875974 | 877038 | Seq. ID No. 3354 |
| 1625c | Seq. ID No. 3355 | 875908 | 875935 | 877038 | Seq. ID No. 3356 |
| 1626a | Seq. ID No. 3357 | 876984 | 877617 | 877994 | Seq. ID No. 3358 |
| 1626b | Seq. ID No. 3359 | 876984 | 877038 | 877994 | Seq. ID No. 3360 |
| 1628a | Seq. ID No. 3361 | 878218 | 878263 | 879225 | Seq. ID No. 3362 |
| 1628b | Seq. ID No. 3363 | 878218 | 878233 | 879225 | Seq. ID No. 3364 |
| 1632a | Seq. ID No. 3365 | 880585 | 880471 | 879908 | Seq. ID No. 3366 |
| 1632b | Seq. ID No. 3367 | 880585 | 880525 | 879908 | Seq. ID No. 3368 |
| 1633a | Seq. ID No. 3369 | 882546 | 882498 | 882007 | Seq. ID No. 3370 |
| 1633b | Seq. ID No. 3371 | 882546 | 882543 | 882007 | Seq. ID No. 3372 |
| 1634a | Seq. ID No. 3373 | 880656 | 880746 | 882020 | Seq. ID No. 3374 |
| 1634b | Seq. ID No. 3375 | 880656 | 880707 | 882020 | Seq. ID No. 3376 |
| 1634c | Seq. ID No. 3377 | 880656 | 880659 | 882020 | Seq. ID No. 3378 |
| 1639b | Seq. ID No. 3379 | 881983 | 881992 | 883659 | Seq. ID No. 3380 |
| 1642a | Seq. ID No. 3381 | 883922 | 883976 | 886069 | Seq. ID No. 3382 |
| 1642b | Seq. ID No. 3383 | 883922 | 883934 | 886069 | Seq. ID No. 3384 |
| 1643a | Seq. ID No. 3385 | 886023 | 886074 | 886400 | Seq. ID No. 3386 |
| 1643b | Seq. ID No. 3387 | 886023 | 886035 | 886400 | Seq. ID No. 3388 |
| 1646a | Seq. ID No. 3389 | 886658 | 888038 | 888331 | Seq. ID No. 3390 |
| 1646b | Seq. ID No. 3391 | 886658 | 887708 | 888331 | Seq. ID No. 3392 |
| 1646c | Seq. ID No. 3393 | 886658 | 886886 | 888331 | Seq. ID No. 3394 |
| 1646d | Seq. ID No. 3395 | 886658 | 886745 | 888331 | Seq. ID No. 3396 |
| 1646e | Seq. ID No. 3397 | 886658 | 886664 | 888331 | Seq. ID No. 3398 |
| 1646f | Seq. ID No. 3399 | 886658 | 886658 | 888331 | Seq. ID No. 3400 |
| 1647 | Seq. ID No. 3401 | 888398 | 888425 | 889144 | Seq. ID No. 3402 |
| 1648b | Seq. ID No. 3403 | 890392 | 890371 | 890099 | Seq. ID No. 3404 |
| 1649a | Seq. ID No. 3405 | 891564 | 891375 | 890626 | Seq. ID No. 3406 |
| 1649b | Seq. ID No. 3407 | 891564 | 891537 | 890626 | Seq. ID No. 3408 |
| 1649c | Seq. ID No. 3409 | 891564 | 891555 | 890626 | Seq. ID No. 3410 |
| 1652a | Seq. ID No. 3411 | 891565 | 891646 | 893049 | Seq. ID No. 3412 |
| 1652b | Seq. ID No. 3413 | 891565 | 891580 | 893049 | Seq. ID No. 3414 |
| 1654 | Seq. ID No. 3415 | 893169 | 893250 | 894092 | Seq. ID No. 3416 |
| 1656b | Seq. ID No. 3417 | 894132 | 894327 | 894650 | Seq. ID No. 3418 |
| 1657 | Seq. ID No. 3419 | 895472 | 895439 | 895203 | Seq. ID No. 3420 |
| 1659a | Seq. ID No. 3421 | 894949 | 896161 | 896691 | Seq. ID No. 3422 |
| 1659b | Seq. ID No. 3423 | 894949 | 895030 | 896691 | Seq. ID No. 3424 |
| 1659c | Seq. ID No. 3425 | 894949 | 894982 | 896691 | Seq. ID No. 3426 |
| 1660b | Seq. ID No. 3427 | 896222 | 896285 | 896725 | Seq. ID No. 3428 |
| 1662a | Seq. ID No. 3429 | 896963 | 896975 | 897847 | Seq. ID No. 3430 |
| 1662b | Seq. ID No. 3431 | 896963 | 896963 | 897847 | Seq. ID No. 3432 |
| 1663 | Seq. ID No. 3433 | 898657 | 898648 | 898361 | Seq. ID No. 3434 |
| 1664 | Seq. ID No. 3435 | 897988 | 898021 | 898602 | Seq. ID No. 3436 |
| 1665a | Seq. ID No. 3437 | 898458 | 898578 | 898913 | Seq. ID No. 3438 |
| 1668 | Seq. ID No. 3439 | 899037 | 899052 | 899945 | Seq. ID No. 3440 |
| 1669 | Seq. ID No. 3441 | 900663 | 900630 | 900307 | Seq. ID No. 3442 |
| 1670a | Seq. ID No. 3443 | 900059 | 900101 | 900757 | Seq. ID No. 3444 |
| 1670b | Seq. ID No. 3445 | 900059 | 900089 | 900757 | Seq. ID No. 3446 |
| 1670c | Seq. ID No. 3447 | 900059 | 900068 | 900757 | Seq. ID No. 3448 |
| 1673a | Seq. ID No. 3449 | 900778 | 902047 | 902286 | Seq. ID No. 3450 |
| 1673b | Seq. ID No. 3451 | 900778 | 901402 | 902286 | Seq. ID No. 3452 |
| 1673c | Seq. ID No. 3453 | 900778 | 901204 | 902286 | Seq. ID No. 3454 |
| 1673d | Seq. ID No. 3455 | 900778 | 901018 | 902286 | Seq. ID No. 3456 |
| 1673e | Seq. ID No. 3457 | 900778 | 900898 | 902286 | Seq. ID No. 3458 |
| 1673f | Seq. ID No. 3459 | 900778 | 900814 | 902286 | Seq. ID No. 3460 |
| 1674 | Seq. ID No. 3461 | 902433 | 902532 | 903383 | Seq. ID No. 3462 |
| 1675a | Seq. ID No. 3463 | 904184 | 904340 | 905383 | Seq. ID No. 3464 |
| 1675b | Seq. ID No. 3465 | 904184 | 904208 | 905383 | Seq. ID No. 3466 |
| 1675c | Seq. ID No. 3467 | 904184 | 904184 | 905383 | Seq. ID No. 3468 |
| 1676 | Seq. ID No. 3469 | 905424 | 905451 | 905852 | Seq. ID No. 3470 |
| 1677 | Seq. ID No. 3471 | 907317 | 907284 | 906022 | Seq. ID No. 3472 |
| 1679 | Seq. ID No. 3473 | 907448 | 907472 | 908014 | Seq. ID No. 3474 |
| 1680 | Seq. ID No. 3475 | 908628 | 908625 | 908176 | Seq. ID No. 3476 |
| 1682a | Seq. ID No. 3477 | 908785 | 908872 | 909591 | Seq. ID No. 3478 |
| 1682b | Seq. ID No. 3479 | 908785 | 908803 | 909591 | Seq. ID No. 3480 |
| 1683a | Seq. ID No. 3481 | 909633 | 910008 | 910523 | Seq. ID No. 3482 |
| 1683b | Seq. ID No. 3483 | 909633 | 909669 | 910523 | Seq. ID No. 3484 |
| 1684 | Seq. ID No. 3485 | 911359 | 911347 | 910640 | Seq. ID No. 3486 |
| 1685 | Seq. ID No. 3487 | 912399 | 912375 | 911350 | Seq. ID No. 3488 |
| 1687 | Seq. ID No. 3489 | 912556 | 912568 | 912813 | Seq. ID No. 3490 |
| 1688a | Seq. ID No. 3491 | 912536 | 913217 | 913645 | Seq. ID No. 3492 |
| 1688b | Seq. ID No. 3493 | 912536 | 912641 | 913645 | Seq. ID No. 3494 |
| 1688c | Seq. ID No. 3495 | 912536 | 912539 | 913645 | Seq. ID No. 3496 |
| 1689a | Seq. ID No. 3497 | 913587 | 913638 | 914393 | Seq. ID No. 3498 |
| 1689b | Seq. ID No. 3499 | 913587 | 913602 | 914393 | Seq. ID No. 3500 |
| 1689c | Seq. ID No. 3501 | 913587 | 913596 | 914393 | Seq. ID No. 3502 |
| 1690a | Seq. ID No. 3503 | 914420 | 914993 | 915481 | Seq. ID No. 3504 |
| 1690b | Seq. ID No. 3505 | 914420 | 914423 | 915481 | Seq. ID No. 3506 |
| 1691 | Seq. ID No. 3507 | 915482 | 915497 | 916570 | Seq. ID No. 3508 |
| 1692 | Seq. ID No. 3509 | 916577 | 916586 | 917389 | Seq. ID No. 3510 |
| 1693 | Seq. ID No. 3511 | 917802 | 917814 | 919235 | Seq. ID No. 3512 |
| 1694a | Seq. ID No. 3513 | 919415 | 919634 | 920410 | Seq. ID No. 3514 |
| 1694b | Seq. ID No. 3515 | 919415 | 919559 | 920410 | Seq. ID No. 3516 |
| 1694c | Seq. ID No. 3517 | 919415 | 919424 | 920410 | Seq. ID No. 3518 |
| 1694d | Seq. ID No. 3519 | 919415 | 919415 | 920410 | Seq. ID No. 3520 |
| 1696 | Seq. ID No. 3521 | 920424 | 920427 | 921608 | Seq. ID No. 3522 |
| 1697a | Seq. ID No. 3523 | 921701 | 921848 | 922666 | Seq. ID No. 3524 |
| 1697b | Seq. ID No. 3525 | 921701 | 921710 | 922666 | Seq. ID No. 3526 |
| 1699a | Seq. ID No. 3527 | 922706 | 923171 | 924190 | Seq. ID No. 3528 |
| 1699b | Seq. ID No. 3529 | 922706 | 922706 | 924190 | Seq. ID No. 3530 |
| 1700a | Seq. ID No. 3531 | 924300 | 924342 | 924713 | Seq. ID No. 3532 |
| 1700b | Seq. ID No. 3533 | 924300 | 924303 | 924713 | Seq. ID No. 3534 |
| 1701a | Seq. ID No. 3535 | 925996 | 925984 | 924812 | Seq. ID No. 3536 |
| 1701b | Seq. ID No. 3537 | 925996 | 925987 | 924812 | Seq. ID No. 3538 |
| 1703b | Seq. ID No. 3539 | 924930 | 924948 | 925280 | Seq. ID No. 3540 |
| 1704a | Seq. ID No. 3541 | 926230 | 926311 | 926853 | Seq. ID No. 3542 |
| 1704b | Seq. ID No. 3543 | 926230 | 926239 | 926853 | Seq. ID No. 3544 |
| 1705a | Seq. ID No. 3545 | 927259 | 927289 | 927513 | Seq. ID No. 3546 |
| 1705b | Seq. ID No. 3547 | 927259 | 927283 | 927513 | Seq. ID No. 3548 |
| 1706 | Seq. ID No. 3549 | 926847 | 926850 | 927800 | Seq. ID No. 3550 |
| 1707a | Seq. ID No. 3551 | 928874 | 928865 | 928023 | Seq. ID No. 3552 |
| 1707b | Seq. ID No. 3553 | 928874 | 928868 | 928023 | Seq. ID No. 3554 |
| 1708 | Seq. ID No. 3555 | 928398 | 928443 | 928799 | Seq. ID No. 3556 |
| 1709 | Seq. ID No. 3557 | 929780 | 929798 | 930049 | Seq. ID No. 3558 |
| 1710a | Seq. ID No. 3559 | 929083 | 929158 | 930066 | Seq. ID No. 3560 |
| 1710b | Seq. ID No. 3561 | 929083 | 929098 | 930066 | Seq. ID No. 3562 |
| 1712a | Seq. ID No. 3563 | 930274 | 930283 | 931269 | Seq. ID No. 3564 |
| 1712b | Seq. ID No. 3565 | 930274 | 930277 | 931269 | Seq. ID No. 3566 |
| 1715a | Seq. ID No. 3567 | 931778 | 931790 | 932497 | Seq. ID No. 3568 |
| 1715b | Seq. ID No. 3569 | 931778 | 931787 | 932497 | Seq. ID No. 3570 |
| 1718a | Seq. ID No. 3571 | 932926 | 932998 | 933222 | Seq. ID No. 3572 |
| 1718b | Seq. ID No. 3573 | 932926 | 932956 | 933222 | Seq. ID No. 3574 |
| 1722 | Seq. ID No. 3575 | 932562 | 932577 | 936251 | Seq. ID No. 3576 |
| 1723a | Seq. ID No. 3577 | 936220 | 936388 | 937206 | Seq. ID No. 3578 |
| 1723b | Seq. ID No. 3579 | 936220 | 936226 | 937206 | Seq. ID No. 3580 |
| 1724 | Seq. ID No. 3581 | 937832 | 937808 | 937542 | Seq. ID No. 3582 |
| 1727 | Seq. ID No. 3583 | 937230 | 937248 | 938735 | Seq. ID No. 3584 |
| 1728 | Seq. ID No. 3585 | 938736 | 938766 | 939101 | Seq. ID No. 3586 |
| 1731 | Seq. ID No. 3587 | 940559 | 940523 | 940092 | Seq. ID No. 3588 |
| 1733 | Seq. ID No. 3589 | 939109 | 939109 | 940608 | Seq. ID No. 3590 |
| 1734 | Seq. ID No. 3591 | 940535 | 940625 | 940846 | Seq. ID No. 3592 |
| 1735a | Seq. ID No. 3593 | 941618 | 941300 | 940989 | Seq. ID No. 3594 |
| 1735b | Seq. ID No. 3595 | 941618 | 941555 | 940989 | Seq. ID No. 3596 |
| 1737a | Seq. ID No. 3597 | 941044 | 941542 | 945477 | Seq. ID No. 3598 |
| 1737b | Seq. ID No. 3599 | 941044 | 941326 | 945477 | Seq. ID No. 3600 |
| 1738a | Seq. ID No. 3601 | 945878 | 946205 | 946741 | Seq. ID No. 3602 |
| 1738b | Seq. ID No. 3603 | 945878 | 945977 | 946741 | Seq. ID No. 3604 |
| 1738c | Seq. ID No. 3605 | 945878 | 945908 | 946741 | Seq. ID No. 3606 |
| 1739 | Seq. ID No. 3607 | 946742 | 946859 | 947188 | Seq. ID No. 3608 |
| 1741 | Seq. ID No. 3609 | 947264 | 947297 | 948463 | Seq. ID No. 3610 |
| 1743 | Seq. ID No. 3611 | 949075 | 949111 | 950652 | Seq. ID No. 3612 |
| 1744a | Seq. ID No. 3613 | 950653 | 951049 | 951951 | Seq. ID No. 3614 |
| 1744b | Seq. ID No. 3615 | 950653 | 950716 | 951951 | Seq. ID No. 3616 |
| 1745 | Seq. ID No. 3617 | 952788 | 952791 | 953084 | Seq. ID No. 3618 |
| 1747 | Seq. ID No. 3619 | 952652 | 952664 | 954949 | Seq. ID No. 3620 |
| 1749a | Seq. ID No. 3621 | 957393 | 957060 | 956695 | Seq. ID No. 3622 |
| 1749b | Seq. ID No. 3623 | 957393 | 957297 | 956695 | Seq. ID No. 3624 |
| 1750a | Seq. ID No. 3625 | 957380 | 957347 | 957060 | Seq. ID No. 3626 |
| 1752a | Seq. ID No. 3627 | 955633 | 956623 | 957144 | Seq. ID No. 3628 |
| 1752b | Seq. ID No. 3629 | 955633 | 955708 | 957144 | Seq. ID No. 3630 |
| 1753 | Seq. ID No. 3631 | 957145 | 957193 | 957429 | Seq. ID No. 3632 |
| 1756a | Seq. ID No. 3633 | 957791 | 957923 | 958780 | Seq. ID No. 3634 |
| 1756b | Seq. ID No. 3635 | 957791 | 957803 | 958780 | Seq. ID No. 3636 |
| 1757a | Seq. ID No. 3637 | 959675 | 959477 | 958860 | Seq. ID No. 3638 |
| 1757b | Seq. ID No. 3639 | 959675 | 959555 | 958860 | Seq. ID No. 3640 |
| 1759 | Seq. ID No. 3641 | 959662 | 959665 | 959976 | Seq. ID No. 3642 |
| 1760 | Seq. ID No. 3643 | 959998 | 960001 | 960330 | Seq. ID No. 3644 |
| 1762 | Seq. ID No. 3645 | 960400 | 960412 | 960936 | Seq. ID No. 3646 |
| 1764a | Seq. ID No. 3647 | 960937 | 960970 | 961668 | Seq. ID No. 3648 |
| 1764b | Seq. ID No. 3649 | 960937 | 960952 | 961668 | Seq. ID No. 3650 |
| 1764c | Seq. ID No. 3651 | 960937 | 960937 | 961668 | Seq. ID No. 3652 |
| 1765 | Seq. ID No. 3653 | 962620 | 962575 | 961790 | Seq. ID No. 3654 |
| 1766 | Seq. ID No. 3655 | 962761 | 962761 | 962645 | Seq. ID No. 3656 |
| 1767a | Seq. ID No. 3657 | 963674 | 963638 | 962793 | Seq. ID No. 3658 |
| 1767b | Seq. ID No. 3659 | 963674 | 963671 | 962793 | Seq. ID No. 3660 |
| 1769a | Seq. ID No. 3661 | 963809 | 964073 | 964522 | Seq. ID No. 3662 |
| 1769b | Seq. ID No. 3663 | 963809 | 963842 | 964522 | Seq. ID No. 3664 |
| 1771 | Seq. ID No. 3665 | 964635 | 964641 | 965921 | Seq. ID No. 3666 |
| 1773 | Seq. ID No. 3667 | 965922 | 965931 | 966497 | Seq. ID No. 3668 |
| 1774 | Seq. ID No. 3669 | 966556 | 966556 | 966882 | Seq. ID No. 3670 |
| 1775a | Seq. ID No. 3671 | 966897 | 967200 | 968219 | Seq. ID No. 3672 |
| 1775b | Seq. ID No. 3673 | 966897 | 967089 | 968219 | Seq. ID No. 3674 |
| 1775c | Seq. ID No. 3675 | 966897 | 966909 | 968219 | Seq. ID No. 3676 |
| 1778a | Seq. ID No. 3677 | 970818 | 970803 | 970543 | Seq. ID No. 3678 |
| 1778b | Seq. ID No. 3679 | 970818 | 970812 | 970543 | Seq. ID No. 3680 |
| 1780 | Seq. ID No. 3681 | 968216 | 968234 | 971017 | Seq. ID No. 3682 |
| 1783a | Seq. ID No. 3683 | 972121 | 972085 | 971618 | Seq. ID No. 3684 |
| 1783b | Seq. ID No. 3685 | 972121 | 972103 | 971618 | Seq. ID No. 3686 |
| 1784a | Seq. ID No. 3687 | 971018 | 971165 | 971623 | Seq. ID No. 3688 |
| 1784b | Seq. ID No. 3689 | 971018 | 971144 | 971623 | Seq. ID No. 3690 |
| 1788a | Seq. ID No. 3691 | 971657 | 972002 | 972955 | Seq. ID No. 3692 |
| 1788b | Seq. ID No. 3693 | 971657 | 971762 | 972955 | Seq. ID No. 3694 |
| 1788c | Seq. ID No. 3695 | 971657 | 971660 | 972955 | Seq. ID No. 3696 |
| 1789a | Seq. ID No. 3697 | 973047 | 973068 | 973802 | Seq. ID No. 3698 |
| 1789b | Seq. ID No. 3699 | 973047 | 973065 | 973802 | Seq. ID No. 3700 |
| 1790a | Seq. ID No. 3701 | 974447 | 974363 | 974136 | Seq. ID No. 3702 |
| 1790b | Seq. ID No. 3703 | 974447 | 974393 | 974136 | Seq. ID No. 3704 |
| 1792a | Seq. ID No. 3705 | 973813 | 973930 | 974529 | Seq. ID No. 3706 |
| 1792b | Seq. ID No. 3707 | 973813 | 973918 | 974529 | Seq. ID No. 3708 |
| 1792c | Seq. ID No. 3709 | 973813 | 973882 | 974529 | Seq. ID No. 3710 |
| 1793a | Seq. ID No. 3711 | 976354 | 976111 | 974645 | Seq. ID No. 3712 |
| 1793b | Seq. ID No. 3713 | 976354 | 976339 | 974645 | Seq. ID No. 3714 |
| 1793c | Seq. ID No. 3715 | 976354 | 976348 | 974645 | Seq. ID No. 3716 |
| 1794a | Seq. ID No. 3717 | 974942 | 975002 | 975253 | Seq. ID No. 3718 |
| 1797a | Seq. ID No. 3719 | 976552 | 976615 | 977007 | Seq. ID No. 3720 |
| 1797b | Seq. ID No. 3721 | 976552 | 976576 | 977007 | Seq. ID No. 3722 |
| 1798a | Seq. ID No. 3723 | 977119 | 977431 | 977703 | Seq. ID No. 3724 |
| 1798b | Seq. ID No. 3725 | 977119 | 977155 | 977703 | Seq. ID No. 3726 |
| 1798c | Seq. ID No. 3727 | 977119 | 977119 | 977703 | Seq. ID No. 3728 |
| 1800 | Seq. ID No. 3729 | 977713 | 977731 | 978411 | Seq. ID No. 3730 |
| 1802a | Seq. ID No. 3731 | 978665 | 978695 | 979249 | Seq. ID No. 3732 |
| 1802b | Seq. ID No. 3733 | 978665 | 978674 | 979249 | Seq. ID No. 3734 |
| 1803 | Seq. ID No. 3735 | 979246 | 979252 | 979512 | Seq. ID No. 3736 |
| 1804a | Seq. ID No. 3737 | 979506 | 979521 | 979817 | Seq. ID No. 3738 |
| 1804b | Seq. ID No. 3739 | 979506 | 979512 | 979817 | Seq. ID No. 3740 |
| 1805 | Seq. ID No. 3741 | 979818 | 979821 | 980378 | Seq. ID No. 3742 |
| 1806a | Seq. ID No. 3743 | 980320 | 980392 | 981078 | Seq. ID No. 3744 |
| 1806b | Seq. ID No. 3745 | 980320 | 980371 | 981078 | Seq. ID No. 3746 |
| 1806c | Seq. ID No. 3747 | 980320 | 980338 | 981078 | Seq. ID No. 3748 |
| 1807a | Seq. ID No. 3749 | 981100 | 981109 | 981456 | Seq. ID No. 3750 |
| 1807b | Seq. ID No. 3751 | 981100 | 981100 | 981456 | Seq. ID No. 3752 |
| 1808a | Seq. ID No. 3753 | 981441 | 982707 | 983054 | Seq. ID No. 3754 |
| 1808b | Seq. ID No. 3755 | 981441 | 982521 | 983054 | Seq. ID No. 3756 |
| 1808c | Seq. ID No. 3757 | 981441 | 982008 | 983054 | Seq. ID No. 3758 |
| 1808d | Seq. ID No. 3759 | 981441 | 981453 | 983054 | Seq. ID No. 3760 |
| 1809a | Seq. ID No. 3761 | 983069 | 984152 | 984700 | Seq. ID No. 3762 |
| 1809b | Seq. ID No. 3763 | 983069 | 983858 | 984700 | Seq. ID No. 3764 |
| 1809c | Seq. ID No. 3765 | 983069 | 983705 | 984700 | Seq. ID No. 3766 |
| 1809d | Seq. ID No. 3767 | 983069 | 983258 | 984700 | Seq. ID No. 3768 |
| 1809e | Seq. ID No. 3769 | 983069 | 983093 | 984700 | Seq. ID No. 3770 |
| 1809f | Seq. ID No. 3771 | 983069 | 983090 | 984700 | Seq. ID No. 3772 |
| 1810a | Seq. ID No. 3773 | 984753 | 984786 | 985220 | Seq. ID No. 3774 |
| 1810b | Seq. ID No. 3775 | 984753 | 984777 | 985220 | Seq. ID No. 3776 |
| 1811a | Seq. ID No. 3777 | 985498 | 985525 | 986454 | Seq. ID No. 3778 |
| 1811b | Seq. ID No. 3779 | 985498 | 985504 | 986454 | Seq. ID No. 3780 |
| 1812a | Seq. ID No. 3781 | 986785 | 986863 | 987144 | Seq. ID No. 3782 |
| 1812b | Seq. ID No. 3783 | 986785 | 986821 | 987144 | Seq. ID No. 3784 |
| 1813 | Seq. ID No. 3785 | 987852 | 987849 | 987478 | Seq. ID No. 3786 |
| 1815a | Seq. ID No. 3787 | 988694 | 988457 | 988041 | Seq. ID No. 3788 |
| 1815b | Seq. ID No. 3789 | 988694 | 988541 | 988041 | Seq. ID No. 3790 |
| 1815c | Seq. ID No. 3791 | 988694 | 988565 | 988041 | Seq. ID No. 3792 |
| 1816a | Seq. ID No. 3793 | 990681 | 990537 | 988399 | Seq. ID No. 3794 |
| 1816b | Seq. ID No. 3795 | 990681 | 990672 | 988399 | Seq. ID No. 3796 |
| 1816c | Seq. ID No. 3797 | 990681 | 990675 | 988399 | Seq. ID No. 3798 |
| 1817a | Seq. ID No. 3799 | 988113 | 988182 | 988472 | Seq. ID No. 3800 |
| 1819a | Seq. ID No. 3801 | 991399 | 991240 | 990737 | Seq. ID No. 3802 |
| 1819b | Seq. ID No. 3803 | 991399 | 991372 | 990737 | Seq. ID No. 3804 |
| 1821 | Seq. ID No. 3805 | 991399 | 991462 | 992019 | Seq. ID No. 3806 |
| 1822 | Seq. ID No. 3807 | 992670 | 992622 | 992392 | Seq. ID No. 3808 |
| 1823a | Seq. ID No. 3809 | 992081 | 992093 | 992791 | Seq. ID No. 3810 |
| 1823b | Seq. ID No. 3811 | 992081 | 992084 | 992791 | Seq. ID No. 3812 |
| 1825 | Seq. ID No. 3813 | 993363 | 993384 | 993830 | Seq. ID No. 3814 |
| 1826a | Seq. ID No. 3815 | 994009 | 994021 | 994287 | Seq. ID No. 3816 |
| 1826b | Seq. ID No. 3817 | 994009 | 994015 | 994287 | Seq. ID No. 3818 |
| 1828 | Seq. ID No. 3819 | 993831 | 993834 | 994970 | Seq. ID No. 3820 |
| 1830a | Seq. ID No. 3821 | 995061 | 995355 | 995987 | Seq. ID No. 3822 |
| 1830b | Seq. ID No. 3823 | 995061 | 995127 | 995987 | Seq. ID No. 3824 |
| 1833 | Seq. ID No. 3825 | 996148 | 996148 | 997719 | Seq. ID No. 3826 |
| 1834 | Seq. ID No. 3827 | 998067 | 998067 | 997864 | Seq. ID No. 3828 |
| 1835 | Seq. ID No. 3829 | 998280 | 998280 | 998462 | Seq. ID No. 3830 |
| 1836a | Seq. ID No. 3831 | 998407 | 998452 | 999252 | Seq. ID No. 3832 |
| 1836b | Seq. ID No. 3833 | 998407 | 998437 | 999252 | Seq. ID No. 3834 |
| 1837 | Seq. ID No. 3835 | 999249 | 999264 | 999962 | Seq. ID No. 3836 |
| 1838a | Seq. ID No. 3837 | 999984 | 1000437 | 1001729 | Seq. ID No. 3838 |
| 1838b | Seq. ID No. 3839 | 999984 | 1000410 | 1001729 | Seq. ID No. 3840 |
| 1838c | Seq. ID No. 3841 | 999984 | 1000026 | 1001729 | Seq. ID No. 3842 |
| 1838d | Seq. ID No. 3843 | 999984 | 999993 | 1001729 | Seq. ID No. 3844 |
| 1839a | Seq. ID No. 3845 | 1001786 | 1001882 | 1002862 | Seq. ID No. 3846 |
| 1839b | Seq. ID No. 3847 | 1001786 | 1001852 | 1002862 | Seq. ID No. 3848 |
| 1842a | Seq. ID No. 3849 | 1003007 | 1004168 | 1004401 | Seq. ID No. 3850 |
| 1842b | Seq. ID No. 3851 | 1003007 | 1003763 | 1004401 | Seq. ID No. 3852 |
| 1842c | Seq. ID No. 3853 | 1003007 | 1003511 | 1004401 | Seq. ID No. 3854 |
| 1842d | Seq. ID No. 3855 | 1003007 | 1003013 | 1004401 | Seq. ID No. 3856 |
| 1843 | Seq. ID No. 3857 | 1004418 | 1004421 | 1005749 | Seq. ID No. 3858 |
| 1845a | Seq. ID No. 3859 | 1008055 | 1007122 | 1006469 | Seq. ID No. 3860 |
| 1845b | Seq. ID No. 3861 | 1008055 | 1007620 | 1006469 | Seq. ID No. 3862 |
| 1845c | Seq. ID No. 3863 | 1008055 | 1008037 | 1006469 | Seq. ID No. 3864 |
| 1848a | Seq. ID No. 3865 | 1009315 | 1009012 | 1008188 | Seq. ID No. 3866 |
| 1848b | Seq. ID No. 3867 | 1009315 | 1009315 | 1008188 | Seq. ID No. 3868 |
| 1850a | Seq. ID No. 3869 | 1009477 | 1009501 | 1010796 | Seq. ID No. 3870 |
| 1850b | Seq. ID No. 3871 | 1009477 | 1009495 | 1010796 | Seq. ID No. 3872 |
| 1850c | Seq. ID No. 3873 | 1009477 | 1009486 | 1010796 | Seq. ID No. 3874 |
| 1853a | Seq. ID No. 3875 | 1010827 | 1010860 | 1011777 | Seq. ID No. 3876 |
| 1853b | Seq. ID No. 3877 | 1010827 | 1010830 | 1011777 | Seq. ID No. 3878 |
| 1854a | Seq. ID No. 3879 | 1011872 | 1011941 | 1012564 | Seq. ID No. 3880 |
| 1854b | Seq. ID No. 3881 | 1011872 | 1011875 | 1012564 | Seq. ID No. 3882 |
| 1855 | Seq. ID No. 3883 | 1012793 | 1012808 | 1013707 | Seq. ID No. 3884 |
| 1856 | Seq. ID No. 3885 | 1013934 | 1014024 | 1014491 | Seq. ID No. 3886 |
| 1857a | Seq. ID No. 3887 | 1016563 | 1015273 | 1014593 | Seq. ID No. 3888 |
| 1857b | Seq. ID No. 3889 | 1016563 | 1016080 | 1014593 | Seq. ID No. 3890 |
| 1857c | Seq. ID No. 3891 | 1016563 | 1016137 | 1014593 | Seq. ID No. 3892 |
| 1857d | Seq. ID No. 3893 | 1016563 | 1016536 | 1014593 | Seq. ID No. 3894 |
| 1858 | Seq. ID No. 3895 | 1015131 | 1015131 | 1014907 | Seq. ID No. 3896 |
| 1859 | Seq. ID No. 3897 | 1016974 | 1016971 | 1016564 | Seq. ID No. 3898 |
| 1861a | Seq. ID No. 3899 | 1017158 | 1017200 | 1019077 | Seq. ID No. 3900 |
| 1861b | Seq. ID No. 3901 | 1017158 | 1017173 | 1019077 | Seq. ID No. 3902 |
| 1862a | Seq. ID No. 3903 | 1019736 | 1019589 | 1019356 | Seq. ID No. 3904 |
| 1863a | Seq. ID No. 3905 | 1019023 | 1019089 | 1019583 | Seq. ID No. 3906 |
| 1863b | Seq. ID No. 3907 | 1019023 | 1019050 | 1019583 | Seq. ID No. 3908 |
| 1864a | Seq. ID No. 3909 | 1019384 | 1019564 | 1019725 | Seq. ID No. 3910 |
| 1865a | Seq. ID No. 3911 | 1020661 | 1020484 | 1019975 | Seq. ID No. 3912 |
| 1865b | Seq. ID No. 3913 | 1020661 | 1020583 | 1019975 | Seq. ID No. 3914 |
| 1868 | Seq. ID No. 3915 | 1020702 | 1020720 | 1021364 | Seq. ID No. 3916 |
| 1869 | Seq. ID No. 3917 | 1021387 | 1021423 | 1021665 | Seq. ID No. 3918 |
| 1870 | Seq. ID No. 3919 | 1021696 | 1021726 | 1022442 | Seq. ID No. 3920 |
| 1873a | Seq. ID No. 3921 | 1022670 | 1022817 | 1024157 | Seq. ID No. 3922 |
| 1873b | Seq. ID No. 3923 | 1022670 | 1022691 | 1024157 | Seq. ID No. 3924 |
| 1873c | Seq. ID No. 3925 | 1022670 | 1022685 | 1024157 | Seq. ID No. 3926 |
| 1875b | Seq. ID No. 3927 | 1024158 | 1024332 | 1024784 | Seq. ID No. 3928 |
| 1875c | Seq. ID No. 3929 | 1024158 | 1024176 | 1024784 | Seq. ID No. 3930 |
| 1877a | Seq. ID No. 3931 | 1024785 | 1024875 | 1025720 | Seq. ID No. 3932 |
| 1877b | Seq. ID No. 3933 | 1024785 | 1024839 | 1025720 | Seq. ID No. 3934 |
| 1878 | Seq. ID No. 3935 | 1025721 | 1025727 | 1026500 | Seq. ID No. 3936 |
| 1879 | Seq. ID No. 3937 | 1026737 | 1026746 | 1027618 | Seq. ID No. 3938 |
| 1882 | Seq. ID No. 3939 | 1029775 | 1029730 | 1029149 | Seq. ID No. 3940 |
| 1884 | Seq. ID No. 3941 | 1027658 | 1027664 | 1029772 | Seq. ID No. 3942 |
| 1886 | Seq. ID No. 3943 | 1030310 | 1030322 | 1031617 | Seq. ID No. 3944 |
| 1887 | Seq. ID No. 3945 | 1032244 | 1032274 | 1033614 | Seq. ID No. 3946 |
| 1888a | Seq. ID No. 3947 | 1033590 | 1033638 | 1034795 | Seq. ID No. 3948 |
| 1888b | Seq. ID No. 3949 | 1033590 | 1033605 | 1034795 | Seq. ID No. 3950 |
| 1890 | Seq. ID No. 3951 | 1035321 | 1035333 | 1036628 | Seq. ID No. 3952 |
| 1891a | Seq. ID No. 3953 | 1036778 | 1036790 | 1037686 | Seq. ID No. 3954 |
| 1891b | Seq. ID No. 3955 | 1036778 | 1036781 | 1037686 | Seq. ID No. 3956 |
| 1893 | Seq. ID No. 3957 | 1037696 | 1037708 | 1038250 | Seq. ID No. 3958 |
| 1894c | Seq. ID No. 3959 | 1039058 | 1039037 | 1038525 | Seq. ID No. 3960 |
| 1895 | Seq. ID No. 3961 | 1039206 | 1039179 | 1038955 | Seq. ID No. 3962 |
| 1896a | Seq. ID No. 3963 | 1039673 | 1039535 | 1039275 | Seq. ID No. 3964 |
| 1896b | Seq. ID No. 3965 | 1039673 | 1039670 | 1039275 | Seq. ID No. 3966 |
| 1897a | Seq. ID No. 3967 | 1038235 | 1038385 | 1039677 | Seq. ID No. 3968 |
| 1897b | Seq. ID No. 3969 | 1038235 | 1038367 | 1039677 | Seq. ID No. 3970 |
| 1897c | Seq. ID No. 3971 | 1038235 | 1038247 | 1039677 | Seq. ID No. 3972 |
| 1898a | Seq. ID No. 3973 | 1040455 | 1040092 | 1039814 | Seq. ID No. 3974 |
| 1898b | Seq. ID No. 3975 | 1040455 | 1040443 | 1039814 | Seq. ID No. 3976 |
| 1899 | Seq. ID No. 3977 | 1039856 | 1039856 | 1039984 | Seq. ID No. 3978 |
| 1900 | Seq. ID No. 3979 | 1040697 | 1040739 | 1041041 | Seq. ID No. 3980 |
| 1902a | Seq. ID No. 3981 | 1040639 | 1040720 | 1042606 | Seq. ID No. 3982 |
| 1902b | Seq. ID No. 3983 | 1040639 | 1040645 | 1042606 | Seq. ID No. 3984 |
| 1907 | Seq. ID No. 3985 | 1042729 | 1042732 | 1045191 | Seq. ID No. 3986 |
| 1912 | Seq. ID No. 3987 | 1045328 | 1045331 | 1047583 | Seq. ID No. 3988 |
| 1915a | Seq. ID No. 3989 | 1047614 | 1047626 | 1048372 | Seq. ID No. 3990 |
| 1915b | Seq. ID No. 3991 | 1047614 | 1047620 | 1048372 | Seq. ID No. 3992 |
| 1916 | Seq. ID No. 3993 | 1048741 | 1048741 | 1048932 | Seq. ID No. 3994 |
| 1917a | Seq. ID No. 3995 | 1049809 | 1049785 | 1049450 | Seq. ID No. 3996 |
| 1917b | Seq. ID No. 3997 | 1049809 | 1049806 | 1049450 | Seq. ID No. 3998 |
| 1918b | Seq. ID No. 3999 | 1049037 | 1049160 | 1049855 | Seq. ID No. 4000 |
| 1918c | Seq. ID No. 4001 | 1049037 | 1049073 | 1049855 | Seq. ID No. 4002 |
| 1918d | Seq. ID No. 4003 | 1049037 | 1049058 | 1049855 | Seq. ID No. 4004 |
| 1919a | Seq. ID No. 4005 | 1050015 | 1050093 | 1050395 | Seq. ID No. 4006 |
| 1919b | Seq. ID No. 4007 | 1050015 | 1050060 | 1050395 | Seq. ID No. 4008 |
| 1919c | Seq. ID No. 4009 | 1050015 | 1050033 | 1050395 | Seq. ID No. 4010 |
| 1923a | Seq. ID No. 4011 | 1050906 | 1050942 | 1051274 | Seq. ID No. 4012 |
| 1923b | Seq. ID No. 4013 | 1050906 | 1050921 | 1051274 | Seq. ID No. 4014 |
| 1925 | Seq. ID No. 4015 | 1051259 | 1051301 | 1053043 | Seq. ID No. 4016 |
| 1926 | Seq. ID No. 4017 | 1056054 | 1056039 | 1053739 | Seq. ID No. 4018 |
| 1927a | Seq. ID No. 4019 | 1057220 | 1056728 | 1056186 | Seq. ID No. 4020 |
| 1927b | Seq. ID No. 4021 | 1057220 | 1057217 | 1056186 | Seq. ID No. 4022 |
| 1930 | Seq. ID No. 4023 | 1057377 | 1057383 | 1058411 | Seq. ID No. 4024 |
| 1931a | Seq. ID No. 4025 | 1059508 | 1058830 | 1058501 | Seq. ID No. 4026 |
| 1931b | Seq. ID No. 4027 | 1059508 | 1058938 | 1058501 | Seq. ID No. 4028 |
| 1931c | Seq. ID No. 4029 | 1059508 | 1059370 | 1058501 | Seq. ID No. 4030 |
| 1932 | Seq. ID No. 4031 | 1059744 | 1059726 | 1059454 | Seq. ID No. 4032 |
| 1933a | Seq. ID No. 4033 | 1060398 | 1060266 | 1059748 | Seq. ID No. 4034 |
| 1933b | Seq. ID No. 4035 | 1060398 | 1060398 | 1059748 | Seq. ID No. 4036 |
| 1934 | Seq. ID No. 4037 | 1061142 | 1061124 | 1060399 | Seq. ID No. 4038 |
| 1936 | Seq. ID No. 4039 | 1062327 | 1062327 | 1061245 | Seq. ID No. 4040 |
| 1937a | Seq. ID No. 4041 | 1063629 | 1062585 | 1062328 | Seq. ID No. 4042 |
| 1937b | Seq. ID No. 4043 | 1063629 | 1062750 | 1062328 | Seq. ID No. 4044 |
| 1937c | Seq. ID No. 4045 | 1063629 | 1062804 | 1062328 | Seq. ID No. 4046 |
| 1937d | Seq. ID No. 4047 | 1063629 | 1063587 | 1062328 | Seq. ID No. 4048 |
| 1937e | Seq. ID No. 4049 | 1063629 | 1063596 | 1062328 | Seq. ID No. 4050 |
| 1939a | Seq. ID No. 4051 | 1064617 | 1064539 | 1063607 | Seq. ID No. 4052 |
| 1939b | Seq. ID No. 4053 | 1064617 | 1064596 | 1063607 | Seq. ID No. 4054 |
| 1939c | Seq. ID No. 4055 | 1064617 | 1064614 | 1063607 | Seq. ID No. 4056 |
| 1940 | Seq. ID No. 4057 | 1065300 | 1065288 | 1064641 | Seq. ID No. 4058 |
| 1941a | Seq. ID No. 4059 | 1066411 | 1066216 | 1065281 | Seq. ID No. 4060 |
| 1941b | Seq. ID No. 4061 | 1066411 | 1066393 | 1065281 | Seq. ID No. 4062 |
| 1942 | Seq. ID No. 4063 | 1067113 | 1067026 | 1066649 | Seq. ID No. 4064 |
| 1943a | Seq. ID No. 4065 | 1067802 | 1067511 | 1067122 | Seq. ID No. 4066 |
| 1943b | Seq. ID No. 4067 | 1067802 | 1067544 | 1067122 | Seq. ID No. 4068 |
| 1944 | Seq. ID No. 4069 | 1067921 | 1067873 | 1067535 | Seq. ID No. 4070 |
| 1946a | Seq. ID No. 4071 | 1069576 | 1068571 | 1068113 | Seq. ID No. 4072 |
| 1946b | Seq. ID No. 4073 | 1069576 | 1069309 | 1068113 | Seq. ID No. 4074 |
| 1946c | Seq. ID No. 4075 | 1069576 | 1069570 | 1068113 | Seq. ID No. 4076 |
| 1949 | Seq. ID No. 4077 | 1069969 | 1069969 | 1069706 | Seq. ID No. 4078 |
| 1950 | Seq. ID No. 4079 | 1070524 | 1070500 | 1069970 | Seq. ID No. 4080 |
| 1951a | Seq. ID No. 4081 | 1072333 | 1072153 | 1070546 | Seq. ID No. 4082 |
| 1951b | Seq. ID No. 4083 | 1072333 | 1072267 | 1070546 | Seq. ID No. 4084 |
| 1951c | Seq. ID No. 4085 | 1072333 | 1072279 | 1070546 | Seq. ID No. 4086 |
| 1951d | Seq. ID No. 4087 | 1072333 | 1072321 | 1070546 | Seq. ID No. 4088 |
| 1953 | Seq. ID No. 4089 | 1073298 | 1073298 | 1072453 | Seq. ID No. 4090 |
| 1954a | Seq. ID No. 4091 | 1074218 | 1074092 | 1073424 | Seq. ID No. 4092 |
| 1954b | Seq. ID No. 4093 | 1074218 | 1074155 | 1073424 | Seq. ID No. 4094 |
| 1954c | Seq. ID No. 4095 | 1074218 | 1074167 | 1073424 | Seq. ID No. 4096 |
| 1956 | Seq. ID No. 4097 | 1074927 | 1074882 | 1074442 | Seq. ID No. 4098 |
| 1957 | Seq. ID No. 4099 | 1075766 | 1075754 | 1074945 | Seq. ID No. 4100 |
| 1958a | Seq. ID No. 4101 | 1076688 | 1076142 | 1075747 | Seq. ID No. 4102 |
| 1958b | Seq. ID No. 4103 | 1076688 | 1076502 | 1075747 | Seq. ID No. 4104 |
| 1958c | Seq. ID No. 4105 | 1076688 | 1076646 | 1075747 | Seq. ID No. 4106 |
| 1960a | Seq. ID No. 4107 | 1077650 | 1077386 | 1076643 | Seq. ID No. 4108 |
| 1960b | Seq. ID No. 4109 | 1077650 | 1077530 | 1076643 | Seq. ID No. 4110 |
| 1960c | Seq. ID No. 4111 | 1077650 | 1077635 | 1076643 | Seq. ID No. 4112 |
| 1961 | Seq. ID No. 4113 | 1076360 | 1076369 | 1076734 | Seq. ID No. 4114 |
| 1962a | Seq. ID No. 4115 | 1079051 | 1079006 | 1077744 | Seq. ID No. 4116 |
| 1962b | Seq. ID No. 4117 | 1079051 | 1079039 | 1077744 | Seq. ID No. 4118 |
| 1965a | Seq. ID No. 4119 | 1080385 | 1080358 | 1079804 | Seq. ID No. 4120 |
| 1965b | Seq. ID No. 4121 | 1080385 | 1080367 | 1079804 | Seq. ID No. 4122 |
| 1966 | Seq. ID No. 4123 | 1080971 | 1080932 | 1080441 | Seq. ID No. 4124 |
| 1967 | Seq. ID No. 4125 | 1081519 | 1081510 | 1081103 | Seq. ID No. 4126 |
| 1968 | Seq. ID No. 4127 | 1082563 | 1082482 | 1081568 | Seq. ID No. 4128 |
| 1969 | Seq. ID No. 4129 | 1082949 | 1082946 | 1082479 | Seq. ID No. 4130 |
| 1970a | Seq. ID No. 4131 | 1083034 | 1083196 | 1083726 | Seq. ID No. 4132 |
| 1970b | Seq. ID No. 4133 | 1083034 | 1083067 | 1083726 | Seq. ID No. 4134 |
| 1972 | Seq. ID No. 4135 | 1084033 | 1084045 | 1084338 | Seq. ID No. 4136 |
| 1973 | Seq. ID No. 4137 | 1083717 | 1083723 | 1084562 | Seq. ID No. 4138 |
| 1976 | Seq. ID No. 4139 | 1085958 | 1085958 | 1085671 | Seq. ID No. 4140 |
| 1977a | Seq. ID No. 4141 | 1084534 | 1084552 | 1085925 | Seq. ID No. 4142 |
| 1977b | Seq. ID No. 4143 | 1084534 | 1084540 | 1085925 | Seq. ID No. 4144 |
| 1977c | Seq. ID No. 4145 | 1084534 | 1084534 | 1085925 | Seq. ID No. 4146 |
| 1979a | Seq. ID No. 4147 | 1090104 | 1089960 | 1086301 | Seq. ID No. 4148 |
| 1979b | Seq. ID No. 4149 | 1090104 | 1090077 | 1086301 | Seq. ID No. 4150 |
| 1980a | Seq. ID No. 4151 | 1088858 | 1088837 | 1088577 | Seq. ID No. 4152 |
| 1980b | Seq. ID No. 4153 | 1088858 | 1088855 | 1088577 | Seq. ID No. 4154 |
| 1981 | Seq. ID No. 4155 | 1089710 | 1089743 | 1090015 | Seq. ID No. 4156 |
| 1982 | Seq. ID No. 4157 | 1093702 | 1093678 | 1090049 | Seq. ID No. 4158 |
| 1985a | Seq. ID No. 4159 | 1095093 | 1094937 | 1093696 | Seq. ID No. 4160 |
| 1985b | Seq. ID No. 4161 | 1095093 | 1095036 | 1093696 | Seq. ID No. 4162 |
| 1986 | Seq. ID No. 4163 | 1096130 | 1096085 | 1094955 | Seq. ID No. 4164 |
| 1987a | Seq. ID No. 4165 | 1096803 | 1096599 | 1096072 | Seq. ID No. 4166 |
| 1987b | Seq. ID No. 4167 | 1096803 | 1096779 | 1096072 | Seq. ID No. 4168 |
| 1989a | Seq. ID No. 4169 | 1097597 | 1097564 | 1096920 | Seq. ID No. 4170 |
| 1989b | Seq. ID No. 4171 | 1097597 | 1097585 | 1096920 | Seq. ID No. 4172 |
| 1989c | Seq. ID No. 4173 | 1097597 | 1097588 | 1096920 | Seq. ID No. 4174 |
| 1990a | Seq. ID No. 4175 | 1098801 | 1098660 | 1097689 | Seq. ID No. 4176 |
| 1990b | Seq. ID No. 4177 | 1098801 | 1098744 | 1097689 | Seq. ID No. 4178 |
| 1990c | Seq. ID No. 4179 | 1098801 | 1098798 | 1097689 | Seq. ID No. 4180 |
| 1991 | Seq. ID No. 4181 | 1098269 | 1098332 | 1098715 | Seq. ID No. 4182 |
| 1992a | Seq. ID No. 4183 | 1100670 | 1100409 | 1098817 | Seq. ID No. 4184 |
| 1992b | Seq. ID No. 4185 | 1100670 | 1100670 | 1098817 | Seq. ID No. 4186 |
| 1993a | Seq. ID No. 4187 | 1103997 | 1103709 | 1100698 | Seq. ID No. 4188 |
| 1993b | Seq. ID No. 4189 | 1103997 | 1103943 | 1100698 | Seq. ID No. 4190 |
| 1993c | Seq. ID No. 4191 | 1103997 | 1103985 | 1100698 | Seq. ID No. 4192 |
| 1996 | Seq. ID No. 4193 | 1106269 | 1106263 | 1104185 | Seq. ID No. 4194 |
| 2000 | Seq. ID No. 4195 | 1107254 | 1107176 | 1106244 | Seq. ID No. 4196 |
| 2001 | Seq. ID No. 4197 | 1108224 | 1108203 | 1107433 | Seq. ID No. 4198 |
| 2003 | Seq. ID No. 4199 | 1109198 | 1109144 | 1108212 | Seq. ID No. 4200 |
| 2006 | Seq. ID No. 4201 | 1109556 | 1109550 | 1109131 | Seq. ID No. 4202 |
| 2008 | Seq. ID No. 4203 | 1109960 | 1109942 | 1109565 | Seq. ID No. 4204 |
| 2010 | Seq. ID No. 4205 | 1110423 | 1110420 | 1109926 | Seq. ID No. 4206 |
| 2011a | Seq. ID No. 4207 | 1111515 | 1111290 | 1110424 | Seq. ID No. 4208 |
| 2011b | Seq. ID No. 4209 | 1111515 | 1111335 | 1110424 | Seq. ID No. 4210 |
| 2011c | Seq. ID No. 4211 | 1111515 | 1111476 | 1110424 | Seq. ID No. 4212 |
| 2013a | Seq. ID No. 4213 | 1112031 | 1111983 | 1111570 | Seq. ID No. 4214 |
| 2013b | Seq. ID No. 4215 | 1112031 | 1112013 | 1111570 | Seq. ID No. 4216 |
| 2014c | Seq. ID No. 4217 | 1112923 | 1112905 | 1112459 | Seq. ID No. 4218 |
| 2015 | Seq. ID No. 4219 | 1112955 | 1112988 | 1113797 | Seq. ID No. 4220 |
| 2016a | Seq. ID No. 4221 | 1115435 | 1114151 | 1113879 | Seq. ID No. 4222 |
| 2016b | Seq. ID No. 4223 | 1115435 | 1114319 | 1113879 | Seq. ID No. 4224 |
| 2016c | Seq. ID No. 4225 | 1115435 | 1114655 | 1113879 | Seq. ID No. 4226 |
| 2016d | Seq. ID No. 4227 | 1115435 | 1115117 | 1113879 | Seq. ID No. 4228 |
| 2016e | Seq. ID No. 4229 | 1115435 | 1115213 | 1113879 | Seq. ID No. 4230 |
| 2016f | Seq. ID No. 4231 | 1115435 | 1115306 | 1113879 | Seq. ID No. 4232 |
| 2016g | Seq. ID No. 4233 | 1115435 | 1115360 | 1113879 | Seq. ID No. 4234 |
| 2016h | Seq. ID No. 4235 | 1115435 | 1115390 | 1113879 | Seq. ID No. 4236 |
| 2020 | Seq. ID No. 4237 | 1115867 | 1115864 | 1115451 | Seq. ID No. 4238 |
| 2021 | Seq. ID No. 4239 | 1116881 | 1116842 | 1115868 | Seq. ID No. 4240 |
| 2023a | Seq. ID No. 4241 | 1117636 | 1117222 | 1116860 | Seq. ID No. 4242 |
| 2023b | Seq. ID No. 4243 | 1117636 | 1117546 | 1116860 | Seq. ID No. 4244 |
| 2024a | Seq. ID No. 4245 | 1120274 | 1120019 | 1117974 | Seq. ID No. 4246 |
| 2024b | Seq. ID No. 4247 | 1120274 | 1120139 | 1117974 | Seq. ID No. 4248 |
| 2024c | Seq. ID No. 4249 | 1120274 | 1120199 | 1117974 | Seq. ID No. 4250 |
| 2024d | Seq. ID No. 4251 | 1120274 | 1120241 | 1117974 | Seq. ID No. 4252 |
| 2025 | Seq. ID No. 4253 | 1117941 | 1117962 | 1118285 | Seq. ID No. 4254 |
| 2027 | Seq. ID No. 4255 | 1120944 | 1120941 | 1120333 | Seq. ID No. 4256 |
| 2030a | Seq. ID No. 4257 | 1121092 | 1121569 | 1121886 | Seq. ID No. 4258 |
| 2030b | Seq. ID No. 4259 | 1121092 | 1121170 | 1121886 | Seq. ID No. 4260 |
| 2030c | Seq. ID No. 4261 | 1121092 | 1121146 | 1121886 | Seq. ID No. 4262 |
| 2030d | Seq. ID No. 4263 | 1121092 | 1121110 | 1121886 | Seq. ID No. 4264 |
| 2032c | Seq. ID No. 4265 | 1122301 | 1122400 | 1122681 | Seq. ID No. 4266 |
| 2033 | Seq. ID No. 4267 | 1121958 | 1121985 | 1122926 | Seq. ID No. 4268 |
| 2035b | Seq. ID No. 4269 | 1124479 | 1124464 | 1124108 | Seq. ID No. 4270 |
| 2036 | Seq. ID No. 4271 | 1123137 | 1123161 | 1124852 | Seq. ID No. 4272 |
| 2037 | Seq. ID No. 4273 | 1126339 | 1126327 | 1125326 | Seq. ID No. 4274 |
| 2038a | Seq. ID No. 4275 | 1126251 | 1126158 | 1125856 | Seq. ID No. 4276 |
| 2040a | Seq. ID No. 4277 | 1125890 | 1125932 | 1126255 | Seq. ID No. 4278 |
| 2042 | Seq. ID No. 4279 | 1126698 | 1126698 | 1127249 | Seq. ID No. 4280 |
| 2043b | Seq. ID No. 4281 | 1127254 | 1127254 | 1128054 | Seq. ID No. 4282 |
| 2044 | Seq. ID No. 4283 | 1129041 | 1129041 | 1128340 | Seq. ID No. 4284 |
| 2045a | Seq. ID No. 4285 | 1128368 | 1128383 | 1128649 | Seq. ID No. 4286 |
| 2045b | Seq. ID No. 4287 | 1128368 | 1128371 | 1128649 | Seq. ID No. 4288 |
| 2046a | Seq. ID No. 4289 | 1130349 | 1130322 | 1129096 | Seq. ID No. 4290 |
| 2046b | Seq. ID No. 4291 | 1130349 | 1130349 | 1129096 | Seq. ID No. 4292 |
| 2047a | Seq. ID No. 4293 | 1131789 | 1131750 | 1130452 | Seq. ID No. 4294 |
| 2047b | Seq. ID No. 4295 | 1131789 | 1131762 | 1130452 | Seq. ID No. 4296 |
| 2049c | Seq. ID No. 4297 | 1132248 | 1132347 | 1132628 | Seq. ID No. 4298 |
| 2050 | Seq. ID No. 4299 | 1131905 | 1131932 | 1132873 | Seq. ID No. 4300 |
| 2053 | Seq. ID No. 4301 | 1133117 | 1133117 | 1134556 | Seq. ID No. 4302 |
| 2054 | Seq. ID No. 4303 | 1135143 | 1135140 | 1134631 | Seq. ID No. 4304 |
| 2055 | Seq. ID No. 4305 | 1135139 | 1135139 | 1135279 | Seq. ID No. 4306 |
| 2056 | Seq. ID No. 4307 | 1135786 | 1135771 | 1135430 | Seq. ID No. 4308 |
| 2057 | Seq. ID No. 4309 | 1137026 | 1137020 | 1135758 | Seq. ID No. 4310 |
| 2059a | Seq. ID No. 4311 | 1137134 | 1137374 | 1138240 | Seq. ID No. 4312 |
| 2059b | Seq. ID No. 4313 | 1137134 | 1137140 | 1138240 | Seq. ID No. 4314 |
| 2060a | Seq. ID No. 4315 | 1138968 | 1138920 | 1138384 | Seq. ID No. 4316 |
| 2060b | Seq. ID No. 4317 | 1138968 | 1138962 | 1138384 | Seq. ID No. 4318 |
| 2062a | Seq. ID No. 4319 | 1140218 | 1140038 | 1139013 | Seq. ID No. 4320 |
| 2062b | Seq. ID No. 4321 | 1140218 | 1140119 | 1139013 | Seq. ID No. 4322 |
| 2062c | Seq. ID No. 4323 | 1140218 | 1140200 | 1139013 | Seq. ID No. 4324 |
| 2064 | Seq. ID No. 4325 | 1141198 | 1141186 | 1140200 | Seq. ID No. 4326 |
| 2065a | Seq. ID No. 4327 | 1142468 | 1142435 | 1141179 | Seq. ID No. 4328 |
| 2065b | Seq. ID No. 4329 | 1142468 | 1142465 | 1141179 | Seq. ID No. 4330 |
| 2066 | Seq. ID No. 4331 | 1143393 | 1143375 | 1142428 | Seq. ID No. 4332 |
| 2068 | Seq. ID No. 4333 | 1145373 | 1145277 | 1143484 | Seq. ID No. 4334 |
| 2069a | Seq. ID No. 4335 | 1146734 | 1146590 | 1145292 | Seq. ID No. 4336 |
| 2069b | Seq. ID No. 4337 | 1146734 | 1146608 | 1145292 | Seq. ID No. 4338 |
| 2069c | Seq. ID No. 4339 | 1146734 | 1146662 | 1145292 | Seq. ID No. 4340 |
| 2074a | Seq. ID No. 4341 | 1147059 | 1147179 | 1148387 | Seq. ID No. 4342 |
| 2074b | Seq. ID No. 4343 | 1147059 | 1147074 | 1148387 | Seq. ID No. 4344 |
| 2075a | Seq. ID No. 4345 | 1149029 | 1148891 | 1148439 | Seq. ID No. 4346 |
| 2075b | Seq. ID No. 4347 | 1149029 | 1148894 | 1148439 | Seq. ID No. 4348 |
| 2076a | Seq. ID No. 4349 | 1151134 | 1151065 | 1148912 | Seq. ID No. 4350 |
| 2076b | Seq. ID No. 4351 | 1151134 | 1151122 | 1148912 | Seq. ID No. 4352 |
| 2076c | Seq. ID No. 4353 | 1151134 | 1151131 | 1148912 | Seq. ID No. 4354 |
| 2078 | Seq. ID No. 4355 | 1151921 | 1151906 | 1151253 | Seq. ID No. 4356 |
| 2079a | Seq. ID No. 4357 | 1152998 | 1152887 | 1151922 | Seq. ID No. 4358 |
| 2079b | Seq. ID No. 4359 | 1152998 | 1152926 | 1151922 | Seq. ID No. 4360 |
| 2079c | Seq. ID No. 4361 | 1152998 | 1152989 | 1151922 | Seq. ID No. 4362 |
| 2081a | Seq. ID No. 4363 | 1153482 | 1153383 | 1152865 | Seq. ID No. 4364 |
| 2081b | Seq. ID No. 4365 | 1153482 | 1153419 | 1152865 | Seq. ID No. 4366 |
| 2086a | Seq. ID No. 4367 | 1153561 | 1153570 | 1154904 | Seq. ID No. 4368 |
| 2086b | Seq. ID No. 4369 | 1153561 | 1153561 | 1154904 | Seq. ID No. 4370 |
| 2087a | Seq. ID No. 4371 | 1154925 | 1154952 | 1155377 | Seq. ID No. 4372 |
| 2087b | Seq. ID No. 4373 | 1154925 | 1154934 | 1155377 | Seq. ID No. 4374 |
| 2088 | Seq. ID No. 4375 | 1156149 | 1156134 | 1155403 | Seq. ID No. 4376 |
| 2091 | Seq. ID No. 4377 | 1156470 | 1156494 | 1157174 | Seq. ID No. 4378 |
| 2092 | Seq. ID No. 4379 | 1157162 | 1157174 | 1157866 | Seq. ID No. 4380 |
| 2093a | Seq. ID No. 4381 | 1157784 | 1157838 | 1158065 | Seq. ID No. 4382 |
| 2094 | Seq. ID No. 4383 | 1158149 | 1158164 | 1158913 | Seq. ID No. 4384 |
| 2096a | Seq. ID No. 4385 | 1158888 | 1159740 | 1160903 | Seq. ID No. 4386 |
| 2096b | Seq. ID No. 4387 | 1158888 | 1159086 | 1160903 | Seq. ID No. 4388 |
| 2096c | Seq. ID No. 4389 | 1158888 | 1158915 | 1160903 | Seq. ID No. 4390 |
| 2096d | Seq. ID No. 4391 | 1158888 | 1158906 | 1160903 | Seq. ID No. 4392 |
| 2097 | Seq. ID No. 4393 | 1163111 | 1163090 | 1161267 | Seq. ID No. 4394 |
| 2098b | Seq. ID No. 4395 | 1161405 | 1161474 | 1161959 | Seq. ID No. 4396 |
| 2100 | Seq. ID No. 4397 | 1164393 | 1164378 | 1163221 | Seq. ID No. 4398 |
| 2101 | Seq. ID No. 4399 | 1164206 | 1164203 | 1163817 | Seq. ID No. 4400 |
| 2104 | Seq. ID No. 4401 | 1166334 | 1166304 | 1164466 | Seq. ID No. 4402 |
| 2105 | Seq. ID No. 4403 | 1164590 | 1164728 | 1164985 | Seq. ID No. 4404 |
| 2107a | Seq. ID No. 4405 | 1164986 | 1165082 | 1165558 | Seq. ID No. 4406 |
| 2107b | Seq. ID No. 4407 | 1164986 | 1165040 | 1165558 | Seq. ID No. 4408 |
| 2109a | Seq. ID No. 4409 | 1166983 | 1166947 | 1166375 | Seq. ID No. 4410 |
| 2109b | Seq. ID No. 4411 | 1166983 | 1166968 | 1166375 | Seq. ID No. 4412 |
| 2110a | Seq. ID No. 4413 | 1168054 | 1168024 | 1166984 | Seq. ID No. 4414 |
| 2110b | Seq. ID No. 4415 | 1168054 | 1168045 | 1166984 | Seq. ID No. 4416 |
| 2110c | Seq. ID No. 4417 | 1168054 | 1168048 | 1166984 | Seq. ID No. 4418 |
| 2112a | Seq. ID No. 4419 | 1169192 | 1168997 | 1168212 | Seq. ID No. 4420 |
| 2112b | Seq. ID No. 4421 | 1169192 | 1169051 | 1168212 | Seq. ID No. 4422 |
| 2112c | Seq. ID No. 4423 | 1169192 | 1169159 | 1168212 | Seq. ID No. 4424 |
| 2114b | Seq. ID No. 4425 | 1168308 | 1168356 | 1168880 | Seq. ID No. 4426 |
| 2115a | Seq. ID No. 4427 | 1170166 | 1170046 | 1169168 | Seq. ID No. 4428 |
| 2115b | Seq. ID No. 4429 | 1170166 | 1170091 | 1169168 | Seq. ID No. 4430 |
| 2115c | Seq. ID No. 4431 | 1170166 | 1170157 | 1169168 | Seq. ID No. 4432 |
| 2116 | Seq. ID No. 4433 | 1170571 | 1170541 | 1170185 | Seq. ID No. 4434 |
| 2117 | Seq. ID No. 4435 | 1172931 | 1172919 | 1170559 | Seq. ID No. 4436 |
| 2120a | Seq. ID No. 4437 | 1170911 | 1170998 | 1171270 | Seq. ID No. 4438 |
| 2120b | Seq. ID No. 4439 | 1170911 | 1170983 | 1171270 | Seq. ID No. 4440 |
| 2122b | Seq. ID No. 4441 | 1172314 | 1172344 | 1172679 | Seq. ID No. 4442 |
| 2123 | Seq. ID No. 4443 | 1173237 | 1173237 | 1172935 | Seq. ID No. 4444 |
| 2124a | Seq. ID No. 4445 | 1173535 | 1173502 | 1173227 | Seq. ID No. 4446 |
| 2124b | Seq. ID No. 4447 | 1173535 | 1173523 | 1173227 | Seq. ID No. 4448 |
| 2125a | Seq. ID No. 4449 | 1174978 | 1174948 | 1173578 | Seq. ID No. 4450 |
| 2125b | Seq. ID No. 4451 | 1174978 | 1174975 | 1173578 | Seq. ID No. 4452 |
| 2127a | Seq. ID No. 4453 | 1174343 | 1174370 | 1174894 | Seq. ID No. 4454 |
| 2127b | Seq. ID No. 4455 | 1174343 | 1174349 | 1174894 | Seq. ID No. 4456 |
| 2128a | Seq. ID No. 4457 | 1175492 | 1175444 | 1174965 | Seq. ID No. 4458 |
| 2128b | Seq. ID No. 4459 | 1175492 | 1175456 | 1174965 | Seq. ID No. 4460 |
| 2133 | Seq. ID No. 4461 | 1179995 | 1179938 | 1175604 | Seq. ID No. 4462 |
| 2138a | Seq. ID No. 4463 | 1181752 | 1181653 | 1180022 | Seq. ID No. 4464 |
| 2138b | Seq. ID No. 4465 | 1181752 | 1181728 | 1180022 | Seq. ID No. 4466 |
| 2138c | Seq. ID No. 4467 | 1181752 | 1181737 | 1180022 | Seq. ID No. 4468 |
| 2140a | Seq. ID No. 4469 | 1183087 | 1182526 | 1181753 | Seq. ID No. 4470 |
| 2140b | Seq. ID No. 4471 | 1183087 | 1183021 | 1181753 | Seq. ID No. 4472 |
| 2142a | Seq. ID No. 4473 | 1183896 | 1183278 | 1183039 | Seq. ID No. 4474 |
| 2142b | Seq. ID No. 4475 | 1183896 | 1183833 | 1183039 | Seq. ID No. 4476 |
| 2144a | Seq. ID No. 4477 | 1184612 | 1184585 | 1183848 | Seq. ID No. 4478 |
| 2144b | Seq. ID No. 4479 | 1184612 | 1184603 | 1183848 | Seq. ID No. 4480 |
| 2146 | Seq. ID No. 4481 | 1185730 | 1185712 | 1184780 | Seq. ID No. 4482 |
| 2147a | Seq. ID No. 4483 | 1186442 | 1185977 | 1185735 | Seq. ID No. 4484 |
| 2147b | Seq. ID No. 4485 | 1186442 | 1186334 | 1185735 | Seq. ID No. 4486 |
| 2147c | Seq. ID No. 4487 | 1186442 | 1186436 | 1185735 | Seq. ID No. 4488 |
| 2149a | Seq. ID No. 4489 | 1187547 | 1187235 | 1186429 | Seq. ID No. 4490 |
| 2149b | Seq. ID No. 4491 | 1187547 | 1187448 | 1186429 | Seq. ID No. 4492 |
| 2150a | Seq. ID No. 4493 | 1186646 | 1186742 | 1187041 | Seq. ID No. 4494 |
| 2150b | Seq. ID No. 4495 | 1186646 | 1186736 | 1187041 | Seq. ID No. 4496 |
| 2150c | Seq. ID No. 4497 | 1186646 | 1186703 | 1187041 | Seq. ID No. 4498 |
| 2152a | Seq. ID No. 4499 | 1188368 | 1188338 | 1188096 | Seq. ID No. 4500 |
| 2153 | Seq. ID No. 4501 | 1189235 | 1189223 | 1188666 | Seq. ID No. 4502 |
| 2155a | Seq. ID No. 4503 | 1189980 | 1189947 | 1189216 | Seq. ID No. 4504 |
| 2155b | Seq. ID No. 4505 | 1189980 | 1189965 | 1189216 | Seq. ID No. 4506 |
| 2156a | Seq. ID No. 4507 | 1190977 | 1190908 | 1190087 | Seq. ID No. 4508 |
| 2156b | Seq. ID No. 4509 | 1190977 | 1190941 | 1190087 | Seq. ID No. 4510 |
| 2156c | Seq. ID No. 4511 | 1190977 | 1190965 | 1190087 | Seq. ID No. 4512 |
| 2159a | Seq. ID No. 4513 | 1190613 | 1190619 | 1190945 | Seq. ID No. 4514 |
| 2159b | Seq. ID No. 4515 | 1190613 | 1190616 | 1190945 | Seq. ID No. 4516 |
| 2160a | Seq. ID No. 4517 | 1191908 | 1191713 | 1191027 | Seq. ID No. 4518 |
| 2160b | Seq. ID No. 4519 | 1191908 | 1191791 | 1191027 | Seq. ID No. 4520 |
| 2160c | Seq. ID No. 4521 | 1191908 | 1191845 | 1191027 | Seq. ID No. 4522 |
| 2160d | Seq. ID No. 4523 | 1191908 | 1191872 | 1191027 | Seq. ID No. 4524 |
| 2161a | Seq. ID No. 4525 | 1190995 | 1191142 | 1191522 | Seq. ID No. 4526 |
| 2161b | Seq. ID No. 4527 | 1190995 | 1191037 | 1191522 | Seq. ID No. 4528 |
| 2161c | Seq. ID No. 4529 | 1190995 | 1191001 | 1191522 | Seq. ID No. 4530 |
| 2164 | Seq. ID No. 4531 | 1191988 | 1192009 | 1192626 | Seq. ID No. 4532 |
| 2165 | Seq. ID No. 4533 | 1192933 | 1192903 | 1192676 | Seq. ID No. 4534 |
| 2166a | Seq. ID No. 4535 | 1194996 | 1194612 | 1193065 | Seq. ID No. 4536 |
| 2166b | Seq. ID No. 4537 | 1194996 | 1194993 | 1193065 | Seq. ID No. 4538 |
| 2169 | Seq. ID No. 4539 | 1195265 | 1195253 | 1195011 | Seq. ID No. 4540 |
| 2171 | Seq. ID No. 4541 | 1195373 | 1195388 | 1196059 | Seq. ID No. 4542 |
| 2172 | Seq. ID No. 4543 | 1196128 | 1196143 | 1196508 | Seq. ID No. 4544 |
| 2173a | Seq. ID No. 4545 | 1197663 | 1197603 | 1196563 | Seq. ID No. 4546 |
| 2173b | Seq. ID No. 4547 | 1197663 | 1197618 | 1196563 | Seq. ID No. 4548 |
| 2174 | Seq. ID No. 4549 | 1198165 | 1198165 | 1197650 | Seq. ID No. 4550 |
| 2175 | Seq. ID No. 4551 | 1197212 | 1197287 | 1197667 | Seq. ID No. 4552 |
| 2176a | Seq. ID No. 4553 | 1200498 | 1200012 | 1198177 | Seq. ID No. 4554 |
| 2176b | Seq. ID No. 4555 | 1200498 | 1200204 | 1198177 | Seq. ID No. 4556 |
| 2176c | Seq. ID No. 4557 | 1200498 | 1200297 | 1198177 | Seq. ID No. 4558 |
| 2176d | Seq. ID No. 4559 | 1200498 | 1200480 | 1198177 | Seq. ID No. 4560 |
| 2177b | Seq. ID No. 4561 | 1199045 | 1199054 | 1199458 | Seq. ID No. 4562 |
| 2179a | Seq. ID No. 4563 | 1201338 | 1201272 | 1200499 | Seq. ID No. 4564 |
| 2179b | Seq. ID No. 4565 | 1201338 | 1201329 | 1200499 | Seq. ID No. 4566 |
| 2180 | Seq. ID No. 4567 | 1202280 | 1202268 | 1201339 | Seq. ID No. 4568 |
| 2181a | Seq. ID No. 4569 | 1203606 | 1203582 | 1202281 | Seq. ID No. 4570 |
| 2181b | Seq. ID No. 4571 | 1203606 | 1203588 | 1202281 | Seq. ID No. 4572 |
| 2182 | Seq. ID No. 4573 | 1203089 | 1203110 | 1203406 | Seq. ID No. 4574 |
| 2183 | Seq. ID No. 4575 | 1204620 | 1204614 | 1203607 | Seq. ID No. 4576 |
| 2185 | Seq. ID No. 4577 | 1205219 | 1205216 | 1204611 | Seq. ID No. 4578 |
| 2186a | Seq. ID No. 4579 | 1206643 | 1206499 | 1205366 | Seq. ID No. 4580 |
| 2186b | Seq. ID No. 4581 | 1206643 | 1206634 | 1205366 | Seq. ID No. 4582 |
| 2189a | Seq. ID No. 4583 | 1208090 | 1207934 | 1206753 | Seq. ID No. 4584 |
| 2189b | Seq. ID No. 4585 | 1208090 | 1208090 | 1206753 | Seq. ID No. 4586 |
| 2190a | Seq. ID No. 4587 | 1206664 | 1206916 | 1207176 | Seq. ID No. 4588 |
| 2190b | Seq. ID No. 4589 | 1206664 | 1206700 | 1207176 | Seq. ID No. 4590 |
| 2193 | Seq. ID No. 4591 | 1209177 | 1209138 | 1208200 | Seq. ID No. 4592 |
| 2194a | Seq. ID No. 4593 | 1209651 | 1209582 | 1209211 | Seq. ID No. 4594 |
| 2194c | Seq. ID No. 4595 | 1209651 | 1209600 | 1209211 | Seq. ID No. 4596 |
| 2196 | Seq. ID No. 4597 | 1210624 | 1210606 | 1209737 | Seq. ID No. 4598 |
| 2197a | Seq. ID No. 4599 | 1213676 | 1212611 | 1210572 | Seq. ID No. 4600 |
| 2197b | Seq. ID No. 4601 | 1213676 | 1213463 | 1210572 | Seq. ID No. 4602 |
| 2197c | Seq. ID No. 4603 | 1213676 | 1213610 | 1210572 | Seq. ID No. 4604 |
| 2201a | Seq. ID No. 4605 | 1215054 | 1214976 | 1214029 | Seq. ID No. 4606 |
| 2201b | Seq. ID No. 4607 | 1215054 | 1214991 | 1214029 | Seq. ID No. 4608 |
| 2203 | Seq. ID No. 4609 | 1215377 | 1215341 | 1215063 | Seq. ID No. 4610 |
| 2204 | Seq. ID No. 4611 | 1216828 | 1216804 | 1215491 | Seq. ID No. 4612 |
| 2206a | Seq. ID No. 4613 | 1218208 | 1218172 | 1216907 | Seq. ID No. 4614 |
| 2206b | Seq. ID No. 4615 | 1218208 | 1218190 | 1216907 | Seq. ID No. 4616 |
| 2208a | Seq. ID No. 4617 | 1217688 | 1217814 | 1218146 | Seq. ID No. 4618 |
| 2208b | Seq. ID No. 4619 | 1217688 | 1217691 | 1218146 | Seq. ID No. 4620 |
| 2208c | Seq. ID No. 4621 | 1217688 | 1217688 | 1218146 | Seq. ID No. 4622 |
| 2209 | Seq. ID No. 4623 | 1218957 | 1218957 | 1218280 | Seq. ID No. 4624 |
| 2212a | Seq. ID No. 4625 | 1219686 | 1219626 | 1218958 | Seq. ID No. 4626 |
| 2212b | Seq. ID No. 4627 | 1219686 | 1219662 | 1218958 | Seq. ID No. 4628 |
| 2215a | Seq. ID No. 4629 | 1221266 | 1220597 | 1219668 | Seq. ID No. 4630 |
| 2215b | Seq. ID No. 4631 | 1221266 | 1221101 | 1219668 | Seq. ID No. 4632 |
| 2216a | Seq. ID No. 4633 | 1221658 | 1221358 | 1221098 | Seq. ID No. 4634 |
| 2216b | Seq. ID No. 4635 | 1221658 | 1221532 | 1221098 | Seq. ID No. 4636 |
| 2218 | Seq. ID No. 4637 | 1222155 | 1222137 | 1221580 | Seq. ID No. 4638 |
| 2219a | Seq. ID No. 4639 | 1222926 | 1222596 | 1222252 | Seq. ID No. 4640 |
| 2219b | Seq. ID No. 4641 | 1222926 | 1222881 | 1222252 | Seq. ID No. 4642 |
| 2219c | Seq. ID No. 4643 | 1222926 | 1222920 | 1222252 | Seq. ID No. 4644 |
| 2220a | Seq. ID No. 4645 | 1223944 | 1223878 | 1223180 | Seq. ID No. 4646 |
| 2220b | Seq. ID No. 4647 | 1223944 | 1223899 | 1223180 | Seq. ID No. 4648 |
| 2220c | Seq. ID No. 4649 | 1223944 | 1223938 | 1223180 | Seq. ID No. 4650 |
| 2221 | Seq. ID No. 4651 | 1224470 | 1224470 | 1223904 | Seq. ID No. 4652 |
| 2222 | Seq. ID No. 4653 | 1225280 | 1225268 | 1224471 | Seq. ID No. 4654 |
| 2223a | Seq. ID No. 4655 | 1225739 | 1225661 | 1225281 | Seq. ID No. 4656 |
| 2223b | Seq. ID No. 4657 | 1225739 | 1225700 | 1225281 | Seq. ID No. 4658 |
| 2225 | Seq. ID No. 4659 | 1225293 | 1225299 | 1225634 | Seq. ID No. 4660 |
| 2226 | Seq. ID No. 4661 | 1226654 | 1226639 | 1225824 | Seq. ID No. 4662 |
| 2227 | Seq. ID No. 4663 | 1227837 | 1227819 | 1226650 | Seq. ID No. 4664 |
| 2228a | Seq. ID No. 4665 | 1228938 | 1228710 | 1228009 | Seq. ID No. 4666 |
| 2228b | Seq. ID No. 4667 | 1228938 | 1228809 | 1228009 | Seq. ID No. 4668 |
| 2228c | Seq. ID No. 4669 | 1228938 | 1228920 | 1228009 | Seq. ID No. 4670 |
| 2231 | Seq. ID No. 4671 | 1229408 | 1229393 | 1228929 | Seq. ID No. 4672 |
| 2232 | Seq. ID No. 4673 | 1230413 | 1230371 | 1229511 | Seq. ID No. 4674 |
| 2234a | Seq. ID No. 4675 | 1232266 | 1232191 | 1230434 | Seq. ID No. 4676 |
| 2234b | Seq. ID No. 4677 | 1232266 | 1232194 | 1230434 | Seq. ID No. 4678 |
| 2234c | Seq. ID No. 4679 | 1232266 | 1232203 | 1230434 | Seq. ID No. 4680 |
| 2236a | Seq. ID No. 4681 | 1231236 | 1231488 | 1231871 | Seq. ID No. 4682 |
| 2236b | Seq. ID No. 4683 | 1231236 | 1231266 | 1231871 | Seq. ID No. 4684 |
| 2238 | Seq. ID No. 4685 | 1233192 | 1233186 | 1232224 | Seq. ID No. 4686 |
| 2239b | Seq. ID No. 4687 | 1232255 | 1232264 | 1232569 | Seq. ID No. 4688 |
| 2240a | Seq. ID No. 4689 | 1236616 | 1236499 | 1233293 | Seq. ID No. 4690 |
| 2240b | Seq. ID No. 4691 | 1236616 | 1236571 | 1233293 | Seq. ID No. 4692 |
| 2240c | Seq. ID No. 4693 | 1236616 | 1236607 | 1233293 | Seq. ID No. 4694 |
| 2243a | Seq. ID No. 4695 | 1234812 | 1234866 | 1235147 | Seq. ID No. 4696 |
| 2244 | Seq. ID No. 4697 | 1237063 | 1237051 | 1236710 | Seq. ID No. 4698 |
| 2245 | Seq. ID No. 4699 | 1236540 | 1236573 | 1236899 | Seq. ID No. 4700 |
| 2246 | Seq. ID No. 4701 | 1237191 | 1237188 | 1236955 | Seq. ID No. 4702 |
| 2248 | Seq. ID No. 4703 | 1237171 | 1237180 | 1237992 | Seq. ID No. 4704 |
| 2249 | Seq. ID No. 4705 | 1240000 | 1239967 | 1238063 | Seq. ID No. 4706 |
| 2253a | Seq. ID No. 4707 | 1240985 | 1240250 | 1239996 | Seq. ID No. 4708 |
| 2253b | Seq. ID No. 4709 | 1240985 | 1240487 | 1239996 | Seq. ID No. 4710 |
| 2253c | Seq. ID No. 4711 | 1240985 | 1240508 | 1239996 | Seq. ID No. 4712 |
| 2253d | Seq. ID No. 4713 | 1240985 | 1240955 | 1239996 | Seq. ID No. 4714 |
| 2255a | Seq. ID No. 4715 | 1241983 | 1241392 | 1240976 | Seq. ID No. 4716 |
| 2255b | Seq. ID No. 4717 | 1241983 | 1241938 | 1240976 | Seq. ID No. 4718 |
| 2256 | Seq. ID No. 4719 | 1242954 | 1242936 | 1241938 | Seq. ID No. 4720 |
| 2257 | Seq. ID No. 4721 | 1241664 | 1241670 | 1241996 | Seq. ID No. 4722 |
| 2259a | Seq. ID No. 4723 | 1243340 | 1243250 | 1242933 | Seq. ID No. 4724 |
| 2259b | Seq. ID No. 4725 | 1243340 | 1243280 | 1242933 | Seq. ID No. 4726 |
| 2260b | Seq. ID No. 4727 | 1243953 | 1243842 | 1243264 | Seq. ID No. 4728 |
| 2260c | Seq. ID No. 4729 | 1243953 | 1243941 | 1243264 | Seq. ID No. 4730 |
| 2262 | Seq. ID No. 4731 | 1244377 | 1244347 | 1244105 | Seq. ID No. 4732 |
| 2263 | Seq. ID No. 4733 | 1245473 | 1245458 | 1244454 | Seq. ID No. 4734 |
| 2264a | Seq. ID No. 4735 | 1244629 | 1244707 | 1244976 | Seq. ID No. 4736 |
| 2264b | Seq. ID No. 4737 | 1244629 | 1244662 | 1244976 | Seq. ID No. 4738 |
| 2267 | Seq. ID No. 4739 | 1247508 | 1247496 | 1245469 | Seq. ID No. 4740 |
| 2268 | Seq. ID No. 4741 | 1246241 | 1246247 | 1246615 | Seq. ID No. 4742 |
| 2269 | Seq. ID No. 4743 | 1249248 | 1249239 | 1247569 | Seq. ID No. 4744 |
| 2270 | Seq. ID No. 4745 | 1249629 | 1249626 | 1249264 | Seq. ID No. 4746 |
| 2272 | Seq. ID No. 4747 | 1250752 | 1250749 | 1250108 | Seq. ID No. 4748 |
| 2273 | Seq. ID No. 4749 | 1251423 | 1251405 | 1250758 | Seq. ID No. 4750 |
| 2274 | Seq. ID No. 4751 | 1252317 | 1252305 | 1251424 | Seq. ID No. 4752 |
| 2275a | Seq. ID No. 4753 | 1254354 | 1254258 | 1252318 | Seq. ID No. 4754 |
| 2275b | Seq. ID No. 4755 | 1254354 | 1254339 | 1252318 | Seq. ID No. 4756 |
| 2275c | Seq. ID No. 4757 | 1254354 | 1254354 | 1252318 | Seq. ID No. 4758 |
| 2276a | Seq. ID No. 4759 | 1255081 | 1254802 | 1254299 | Seq. ID No. 4760 |
| 2276b | Seq. ID No. 4761 | 1255081 | 1254958 | 1254299 | Seq. ID No. 4762 |
| 2276c | Seq. ID No. 4763 | 1255081 | 1255066 | 1254299 | Seq. ID No. 4764 |
| 2276d | Seq. ID No. 4765 | 1255081 | 1255075 | 1254299 | Seq. ID No. 4766 |
| 2277a | Seq. ID No. 4767 | 1256478 | 1256451 | 1255072 | Seq. ID No. 4768 |
| 2277b | Seq. ID No. 4769 | 1256478 | 1256463 | 1255072 | Seq. ID No. 4770 |
| 2278a | Seq. ID No. 4771 | 1257418 | 1256983 | 1256438 | Seq. ID No. 4772 |
| 2278b | Seq. ID No. 4773 | 1257418 | 1257376 | 1256438 | Seq. ID No. 4774 |
| 2278c | Seq. ID No. 4775 | 1257418 | 1257394 | 1256438 | Seq. ID No. 4776 |
| 2278d | Seq. ID No. 4777 | 1257418 | 1257403 | 1256438 | Seq. ID No. 4778 |
| 2280 | Seq. ID No. 4779 | 1259834 | 1259834 | 1257408 | Seq. ID No. 4780 |
| 2284 | Seq. ID No. 4781 | 1261069 | 1261063 | 1259858 | Seq. ID No. 4782 |
| 2285 | Seq. ID No. 4783 | 1261477 | 1261465 | 1261241 | Seq. ID No. 4784 |
| 2286a | Seq. ID No. 4785 | 1262148 | 1261971 | 1261465 | Seq. ID No. 4786 |
| 2286b | Seq. ID No. 4787 | 1262148 | 1262130 | 1261465 | Seq. ID No. 4788 |
| 2288 | Seq. ID No. 4789 | 1263490 | 1263487 | 1262135 | Seq. ID No. 4790 |
| 2290 | Seq. ID No. 4791 | 1264398 | 1264398 | 1263730 | Seq. ID No. 4792 |
| 2291a | Seq. ID No. 4793 | 1265817 | 1265784 | 1264405 | Seq. ID No. 4794 |
| 2291b | Seq. ID No. 4795 | 1265817 | 1265799 | 1264405 | Seq. ID No. 4796 |
| 2291c | Seq. ID No. 4797 | 1265817 | 1265811 | 1264405 | Seq. ID No. 4798 |
| 2292a | Seq. ID No. 4799 | 1267633 | 1267510 | 1265777 | Seq. ID No. 4800 |
| 2292b | Seq. ID No. 4801 | 1267633 | 1267570 | 1265777 | Seq. ID No. 4802 |
| 2292c | Seq. ID No. 4803 | 1267633 | 1267573 | 1265777 | Seq. ID No. 4804 |
| 2294 | Seq. ID No. 4805 | 1266308 | 1266353 | 1266808 | Seq. ID No. 4806 |
| 2297 | Seq. ID No. 4807 | 1267892 | 1267889 | 1267578 | Seq. ID No. 4808 |
| 2298 | Seq. ID No. 4809 | 1268895 | 1268883 | 1267879 | Seq. ID No. 4810 |
| 2299 | Seq. ID No. 4811 | 1269489 | 1269483 | 1268896 | Seq. ID No. 4812 |
| 2300 | Seq. ID No. 4813 | 1270011 | 1269993 | 1269514 | Seq. ID No. 4814 |
| 2301 | Seq. ID No. 4815 | 1271985 | 1271964 | 1270018 | Seq. ID No. 4816 |
| 2302 | Seq. ID No. 4817 | 1269785 | 1269794 | 1270087 | Seq. ID No. 4818 |
| 2303 | Seq. ID No. 4819 | 1270295 | 1270301 | 1270528 | Seq. ID No. 4820 |
| 2304 | Seq. ID No. 4821 | 1272307 | 1272277 | 1271951 | Seq. ID No. 4822 |
| 2306c | Seq. ID No. 4823 | 1272365 | 1272404 | 1272844 | Seq. ID No. 4824 |
| 2307 | Seq. ID No. 4825 | 1273700 | 1273679 | 1272978 | Seq. ID No. 4826 |
| 2308 | Seq. ID No. 4827 | 1274658 | 1274640 | 1273681 | Seq. ID No. 4828 |
| 2309a | Seq. ID No. 4829 | 1275133 | 1274938 | 1274654 | Seq. ID No. 4830 |
| 2309b | Seq. ID No. 4831 | 1275133 | 1274956 | 1274654 | Seq. ID No. 4832 |
| 2309c | Seq. ID No. 4833 | 1275133 | 1275010 | 1274654 | Seq. ID No. 4834 |
| 2310 | Seq. ID No. 4835 | 1275318 | 1275294 | 1275058 | Seq. ID No. 4836 |
| 2311 | Seq. ID No. 4837 | 1276504 | 1276489 | 1275308 | Seq. ID No. 4838 |
| 2312a | Seq. ID No. 4839 | 1278512 | 1278431 | 1276806 | Seq. ID No. 4840 |
| 2312b | Seq. ID No. 4841 | 1278512 | 1278485 | 1276806 | Seq. ID No. 4842 |
| 2312c | Seq. ID No. 4843 | 1278512 | 1278494 | 1276806 | Seq. ID No. 4844 |
| 2314 | Seq. ID No. 4845 | 1278600 | 1278633 | 1279616 | Seq. ID No. 4846 |
| 2315 | Seq. ID No. 4847 | 1279974 | 1279980 | 1280267 | Seq. ID No. 4848 |
| 2316 | Seq. ID No. 4849 | 1282453 | 1282450 | 1280330 | Seq. ID No. 4850 |
| 2317 | Seq. ID No. 4851 | 1280784 | 1280841 | 1281170 | Seq. ID No. 4852 |
| 2320 | Seq. ID No. 4853 | 1282272 | 1282278 | 1282565 | Seq. ID No. 4854 |
| 2321a | Seq. ID No. 4855 | 1282966 | 1282873 | 1282604 | Seq. ID No. 4856 |
| 2321b | Seq. ID No. 4857 | 1282966 | 1282909 | 1282604 | Seq. ID No. 4858 |
| 2321c | Seq. ID No. 4859 | 1282966 | 1282939 | 1282604 | Seq. ID No. 4860 |
| 2322 | Seq. ID No. 4861 | 1285049 | 1285034 | 1283040 | Seq. ID No. 4862 |
| 2324 | Seq. ID No. 4863 | 1283044 | 1283068 | 1283607 | Seq. ID No. 4864 |
| 2327 | Seq. ID No. 4865 | 1285600 | 1285549 | 1285298 | Seq. ID No. 4866 |
| 2328 | Seq. ID No. 4867 | 1286689 | 1286668 | 1285637 | Seq. ID No. 4868 |
| 2329a | Seq. ID No. 4869 | 1289009 | 1287596 | 1286658 | Seq. ID No. 4870 |
| 2329b | Seq. ID No. 4871 | 1289009 | 1287857 | 1286658 | Seq. ID No. 4872 |
| 2329c | Seq. ID No. 4873 | 1289009 | 1288268 | 1286658 | Seq. ID No. 4874 |
| 2329d | Seq. ID No. 4875 | 1289009 | 1288994 | 1286658 | Seq. ID No. 4876 |
| 2330 | Seq. ID No. 4877 | 1289504 | 1289489 | 1289010 | Seq. ID No. 4878 |
| 2331 | Seq. ID No. 4879 | 1290204 | 1290183 | 1289548 | Seq. ID No. 4880 |
| 2333 | Seq. ID No. 4881 | 1290847 | 1290847 | 1290371 | Seq. ID No. 4882 |
| 2335 | Seq. ID No. 4883 | 1291411 | 1291402 | 1290848 | Seq. ID No. 4884 |
| 2336 | Seq. ID No. 4885 | 1291822 | 1291732 | 1291412 | Seq. ID No. 4886 |
| 2337b | Seq. ID No. 4887 | 1292097 | 1292079 | 1291810 | Seq. ID No. 4888 |
| 2338a | Seq. ID No. 4889 | 1293987 | 1293840 | 1292143 | Seq. ID No. 4890 |
| 2338b | Seq. ID No. 4891 | 1293987 | 1293891 | 1292143 | Seq. ID No. 4892 |
| 2338c | Seq. ID No. 4893 | 1293987 | 1293972 | 1292143 | Seq. ID No. 4894 |
| 2342a | Seq. ID No. 4895 | 1294892 | 1294544 | 1294074 | Seq. ID No. 4896 |
| 2342b | Seq. ID No. 4897 | 1294892 | 1294781 | 1294074 | Seq. ID No. 4898 |
| 2342c | Seq. ID No. 4899 | 1294892 | 1294856 | 1294074 | Seq. ID No. 4900 |
| 2344 | Seq. ID No. 4901 | 1296464 | 1296425 | 1295013 | Seq. ID No. 4902 |
| 2345 | Seq. ID No. 4903 | 1297883 | 1297877 | 1296513 | Seq. ID No. 4904 |
| 2347 | Seq. ID No. 4905 | 1298903 | 1298897 | 1297914 | Seq. ID No. 4906 |
| 2348 | Seq. ID No. 4907 | 1300105 | 1300084 | 1298900 | Seq. ID No. 4908 |
| 2349a | Seq. ID No. 4909 | 1300346 | 1300391 | 1300954 | Seq. ID No. 4910 |
| 2349b | Seq. ID No. 4911 | 1300346 | 1300373 | 1300954 | Seq. ID No. 4912 |
| 2350a | Seq. ID No. 4913 | 1302119 | 1302026 | 1301769 | Seq. ID No. 4914 |
| 2353 | Seq. ID No. 4915 | 1301242 | 1301248 | 1302921 | Seq. ID No. 4916 |
| 2354 | Seq. ID No. 4917 | 1303618 | 1303603 | 1302971 | Seq. ID No. 4918 |
| 2357 | Seq. ID No. 4919 | 1304669 | 1304657 | 1303596 | Seq. ID No. 4920 |
| 2358 | Seq. ID No. 4921 | 1305415 | 1305397 | 1304654 | Seq. ID No. 4922 |
| 2360 | Seq. ID No. 4923 | 1306366 | 1306351 | 1306055 | Seq. ID No. 4924 |
| 2361a | Seq. ID No. 4925 | 1306811 | 1306532 | 1306110 | Seq. ID No. 4926 |
| 2361b | Seq. ID No. 4927 | 1306811 | 1306745 | 1306110 | Seq. ID No. 4928 |
| 2363a | Seq. ID No. 4929 | 1307978 | 1307936 | 1306833 | Seq. ID No. 4930 |
| 2363b | Seq. ID No. 4931 | 1307978 | 1307975 | 1306833 | Seq. ID No. 4932 |
| 2365a | Seq. ID No. 4933 | 1308165 | 1308219 | 1309355 | Seq. ID No. 4934 |
| 2365b | Seq. ID No. 4935 | 1308165 | 1308189 | 1309355 | Seq. ID No. 4936 |
| 2366a | Seq. ID No. 4937 | 1310189 | 1310135 | 1309491 | Seq. ID No. 4938 |
| 2366b | Seq. ID No. 4939 | 1310189 | 1310138 | 1309491 | Seq. ID No. 4940 |
| 2367 | Seq. ID No. 4941 | 1310796 | 1310793 | 1310128 | Seq. ID No. 4942 |
| 2368 | Seq. ID No. 4943 | 1311881 | 1311860 | 1310793 | Seq. ID No. 4944 |
| 2369 | Seq. ID No. 4945 | 1313031 | 1313028 | 1311862 | Seq. ID No. 4946 |
| 2370 | Seq. ID No. 4947 | 1313797 | 1313788 | 1313054 | Seq. ID No. 4948 |
| 2371a | Seq. ID No. 4949 | 1312950 | 1313103 | 1313339 | Seq. ID No. 4950 |
| 2372 | Seq. ID No. 4951 | 1313340 | 1313382 | 1313705 | Seq. ID No. 4952 |
| 2373 | Seq. ID No. 4953 | 1314523 | 1314511 | 1313798 | Seq. ID No. 4954 |
| 2374a | Seq. ID No. 4955 | 1315386 | 1315314 | 1314508 | Seq. ID No. 4956 |
| 2374b | Seq. ID No. 4957 | 1315386 | 1315377 | 1314508 | Seq. ID No. 4958 |
| 2376 | Seq. ID No. 4959 | 1316374 | 1316374 | 1315394 | Seq. ID No. 4960 |
| 2377a | Seq. ID No. 4961 | 1317653 | 1317590 | 1316367 | Seq. ID No. 4962 |
| 2377b | Seq. ID No. 4963 | 1317653 | 1317650 | 1316367 | Seq. ID No. 4964 |
| 2378 | Seq. ID No. 4965 | 1318340 | 1318334 | 1318038 | Seq. ID No. 4966 |
| 2379a | Seq. ID No. 4967 | 1318807 | 1318741 | 1318331 | Seq. ID No. 4968 |
| 2379b | Seq. ID No. 4969 | 1318807 | 1318750 | 1318331 | Seq. ID No. 4970 |
| 2380 | Seq. ID No. 4971 | 1319037 | 1319019 | 1318747 | Seq. ID No. 4972 |
| 2381 | Seq. ID No. 4973 | 1320232 | 1320205 | 1319264 | Seq. ID No. 4974 |
| 2382c | Seq. ID No. 4975 | 1319889 | 1319790 | 1319509 | Seq. ID No. 4976 |
| 2384a | Seq. ID No. 4977 | 1322997 | 1322961 | 1320310 | Seq. ID No. 4978 |
| 2384b | Seq. ID No. 4979 | 1322997 | 1322982 | 1320310 | Seq. ID No. 4980 |
| 2386b | Seq. ID No. 4981 | 1322660 | 1322696 | 1323103 | Seq. ID No. 4982 |
| 2386c | Seq. ID No. 4983 | 1322660 | 1322675 | 1323103 | Seq. ID No. 4984 |
| 2387 | Seq. ID No. 4985 | 1324321 | 1324225 | 1323305 | Seq. ID No. 4986 |
| 2390 | Seq. ID No. 4987 | 1324802 | 1324754 | 1324458 | Seq. ID No. 4988 |
| 2391 | Seq. ID No. 4989 | 1324273 | 1324294 | 1325208 | Seq. ID No. 4990 |
| 2392 | Seq. ID No. 4991 | 1327683 | 1327665 | 1325251 | Seq. ID No. 4992 |
| 2394 | Seq. ID No. 4993 | 1325873 | 1325882 | 1326148 | Seq. ID No. 4994 |
| 2395 | Seq. ID No. 4995 | 1328666 | 1328639 | 1327779 | Seq. ID No. 4996 |
| 2396 | Seq. ID No. 4997 | 1329927 | 1329906 | 1328779 | Seq. ID No. 4998 |
| 2397 | Seq. ID No. 4999 | 1328731 | 1328752 | 1329045 | Seq. ID No. 5000 |
| 2398 | Seq. ID No. 5001 | 1330390 | 1330381 | 1330037 | Seq. ID No. 5002 |
| 2399a | Seq. ID No. 5003 | 1331563 | 1331224 | 1330391 | Seq. ID No. 5004 |
| 2399b | Seq. ID No. 5005 | 1331563 | 1331488 | 1330391 | Seq. ID No. 5006 |
| 2399c | Seq. ID No. 5007 | 1331563 | 1331542 | 1330391 | Seq. ID No. 5008 |
| 2401 | Seq. ID No. 5009 | 1333918 | 1333894 | 1331564 | Seq. ID No. 5010 |
| 2402a | Seq. ID No. 5011 | 1334374 | 1334266 | 1334003 | Seq. ID No. 5012 |
| 2402b | Seq. ID No. 5013 | 1334374 | 1334305 | 1334003 | Seq. ID No. 5014 |
| 2403a | Seq. ID No. 5015 | 1333974 | 1334010 | 1335254 | Seq. ID No. 5016 |
| 2403b | Seq. ID No. 5017 | 1333974 | 1333986 | 1335254 | Seq. ID No. 5018 |
| 2404a | Seq. ID No. 5019 | 1337114 | 1336970 | 1335393 | Seq. ID No. 5020 |
| 2404b | Seq. ID No. 5021 | 1337114 | 1337108 | 1335393 | Seq. ID No. 5022 |
| 2407a | Seq. ID No. 5023 | 1336219 | 1336225 | 1336524 | Seq. ID No. 5024 |
| 2409a | Seq. ID No. 5025 | 1337582 | 1337615 | 1338046 | Seq. ID No. 5026 |
| 2409b | Seq. ID No. 5027 | 1337582 | 1337606 | 1338046 | Seq. ID No. 5028 |
| 2410 | Seq. ID No. 5029 | 1339429 | 1339369 | 1338176 | Seq. ID No. 5030 |
| 2412 | Seq. ID No. 5031 | 1339044 | 1339017 | 1338787 | Seq. ID No. 5032 |
| 2413a | Seq. ID No. 5033 | 1338351 | 1338411 | 1338818 | Seq. ID No. 5034 |
| 2413b | Seq. ID No. 5035 | 1338351 | 1338357 | 1338818 | Seq. ID No. 5036 |
| 2415a | Seq. ID No. 5037 | 1339712 | 1339586 | 1339392 | Seq. ID No. 5038 |
| 2415b | Seq. ID No. 5039 | 1339712 | 1339688 | 1339392 | Seq. ID No. 5040 |
| 2416b | Seq. ID No. 5041 | 1338897 | 1338969 | 1339433 | Seq. ID No. 5042 |
| 2416c | Seq. ID No. 5043 | 1338897 | 1338915 | 1339433 | Seq. ID No. 5044 |
| 2417a | Seq. ID No. 5045 | 1340413 | 1340371 | 1339583 | Seq. ID No. 5046 |
| 2417b | Seq. ID No. 5047 | 1340413 | 1340398 | 1339583 | Seq. ID No. 5048 |
| 2418a | Seq. ID No. 5049 | 1340847 | 1340808 | 1340524 | Seq. ID No. 5050 |
| 2418b | Seq. ID No. 5051 | 1340847 | 1340817 | 1340524 | Seq. ID No. 5052 |
| 2420 | Seq. ID No. 5053 | 1341166 | 1341157 | 1340795 | Seq. ID No. 5054 |
| 2421 | Seq. ID No. 5055 | 1341520 | 1341478 | 1341170 | Seq. ID No. 5056 |
| 2422b | Seq. ID No. 5057 | 1342395 | 1342347 | 1341700 | Seq. ID No. 5058 |
| 2422c | Seq. ID No. 5059 | 1342395 | 1342395 | 1341700 | Seq. ID No. 5060 |
| 2423a | Seq. ID No. 5061 | 1343009 | 1342928 | 1342413 | Seq. ID No. 5062 |
| 2423b | Seq. ID No. 5063 | 1343009 | 1342994 | 1342413 | Seq. ID No. 5064 |
| 2424a | Seq. ID No. 5065 | 1343829 | 1343793 | 1342930 | Seq. ID No. 5066 |
| 2424b | Seq. ID No. 5067 | 1343829 | 1343799 | 1342930 | Seq. ID No. 5068 |
| 2424c | Seq. ID No. 5069 | 1343829 | 1343817 | 1342930 | Seq. ID No. 5070 |
| 2426 | Seq. ID No. 5071 | 1344336 | 1344336 | 1344130 | Seq. ID No. 5072 |
| 2428a | Seq. ID No. 5073 | 1344573 | 1344774 | 1345985 | Seq. ID No. 5074 |
| 2428b | Seq. ID No. 5075 | 1344573 | 1344648 | 1345985 | Seq. ID No. 5076 |
| 2428c | Seq. ID No. 5077 | 1344573 | 1344579 | 1345985 | Seq. ID No. 5078 |
| 2429 | Seq. ID No. 5079 | 1346793 | 1346718 | 1346035 | Seq. ID No. 5080 |
| 2432 | Seq. ID No. 5081 | 1347442 | 1347430 | 1346780 | Seq. ID No. 5082 |
| 2433 | Seq. ID No. 5083 | 1350107 | 1350095 | 1347432 | Seq. ID No. 5084 |
| 2434 | Seq. ID No. 5085 | 1347436 | 1347457 | 1347729 | Seq. ID No. 5086 |
| 2440 | Seq. ID No. 5087 | 1351640 | 1351607 | 1350387 | Seq. ID No. 5088 |
| 2442a | Seq. ID No. 5089 | 1352776 | 1352596 | 1351619 | Seq. ID No. 5090 |
| 2442b | Seq. ID No. 5091 | 1352776 | 1352761 | 1351619 | Seq. ID No. 5092 |
| 2443 | Seq. ID No. 5093 | 1353214 | 1353199 | 1352876 | Seq. ID No. 5094 |
| 2444a | Seq. ID No. 5095 | 1354016 | 1353962 | 1353192 | Seq. ID No. 5096 |
| 2444b | Seq. ID No. 5097 | 1354016 | 1353977 | 1353192 | Seq. ID No. 5098 |
| 2444c | Seq. ID No. 5099 | 1354016 | 1354001 | 1353192 | Seq. ID No. 5100 |
| 2444d | Seq. ID No. 5101 | 1354016 | 1354007 | 1353192 | Seq. ID No. 5102 |
| 2445 | Seq. ID No. 5103 | 1354599 | 1354581 | 1353931 | Seq. ID No. 5104 |
| 2448b | Seq. ID No. 5105 | 1354686 | 1354710 | 1355162 | Seq. ID No. 5106 |
| 2449a | Seq. ID No. 5107 | 1355925 | 1355691 | 1355272 | Seq. ID No. 5108 |
| 2449b | Seq. ID No. 5109 | 1355925 | 1355877 | 1355272 | Seq. ID No. 5110 |
| 2451 | Seq. ID No. 5111 | 1356746 | 1356734 | 1356105 | Seq. ID No. 5112 |
| 2452 | Seq. ID No. 5113 | 1356996 | 1357029 | 1358138 | Seq. ID No. 5114 |
| 2453 | Seq. ID No. 5115 | 1359295 | 1359286 | 1358234 | Seq. ID No. 5116 |
| 2455 | Seq. ID No. 5117 | 1360072 | 1360054 | 1359296 | Seq. ID No. 5118 |
| 2456a | Seq. ID No. 5119 | 1361028 | 1360605 | 1360051 | Seq. ID No. 5120 |
| 2456b | Seq. ID No. 5121 | 1361028 | 1360677 | 1360051 | Seq. ID No. 5122 |
| 2456c | Seq. ID No. 5123 | 1361028 | 1360941 | 1360051 | Seq. ID No. 5124 |
| 2456d | Seq. ID No. 5125 | 1361028 | 1361013 | 1360051 | Seq. ID No. 5126 |
| 2456e | Seq. ID No. 5127 | 1361028 | 1361025 | 1360051 | Seq. ID No. 5128 |
| 2458a | Seq. ID No. 5129 | 1362001 | 1361608 | 1361003 | Seq. ID No. 5130 |
| 2458b | Seq. ID No. 5131 | 1362001 | 1361680 | 1361003 | Seq. ID No. 5132 |
| 2458c | Seq. ID No. 5133 | 1362001 | 1361764 | 1361003 | Seq. ID No. 5134 |
| 2458d | Seq. ID No. 5135 | 1362001 | 1361767 | 1361003 | Seq. ID No. 5136 |
| 2458e | Seq. ID No. 5137 | 1362001 | 1361854 | 1361003 | Seq. ID No. 5138 |
| 2458f | Seq. ID No. 5139 | 1362001 | 1361983 | 1361003 | Seq. ID No. 5140 |
| 2458g | Seq. ID No. 5141 | 1362001 | 1361986 | 1361003 | Seq. ID No. 5142 |
| 2461a | Seq. ID No. 5143 | 1363565 | 1363550 | 1362201 | Seq. ID No. 5144 |
| 2461b | Seq. ID No. 5145 | 1363565 | 1363556 | 1362201 | Seq. ID No. 5146 |
| 2462a | Seq. ID No. 5147 | 1364408 | 1364270 | 1363584 | Seq. ID No. 5148 |
| 2462b | Seq. ID No. 5149 | 1364408 | 1364294 | 1363584 | Seq. ID No. 5150 |
| 2463a | Seq. ID No. 5151 | 1363279 | 1363366 | 1363731 | Seq. ID No. 5152 |
| 2464 | Seq. ID No. 5153 | 1364536 | 1364536 | 1364309 | Seq. ID No. 5154 |
| 2465a | Seq. ID No. 5155 | 1365094 | 1365082 | 1364537 | Seq. ID No. 5156 |
| 2465b | Seq. ID No. 5157 | 1365094 | 1365094 | 1364537 | Seq. ID No. 5158 |
| 2468a | Seq. ID No. 5159 | 1365781 | 1365751 | 1365422 | Seq. ID No. 5160 |
| 2468b | Seq. ID No. 5161 | 1365781 | 1365775 | 1365422 | Seq. ID No. 5162 |
| 2469a | Seq. ID No. 5163 | 1365183 | 1365474 | 1365908 | Seq. ID No. 5164 |
| 2469b | Seq. ID No. 5165 | 1365183 | 1365204 | 1365908 | Seq. ID No. 5166 |
| 2470 | Seq. ID No. 5167 | 1367011 | 1367011 | 1366664 | Seq. ID No. 5168 |
| 2471 | Seq. ID No. 5169 | 1365926 | 1365932 | 1366858 | Seq. ID No. 5170 |
| 2473 | Seq. ID No. 5171 | 1366716 | 1366872 | 1367141 | Seq. ID No. 5172 |
| 2474a | Seq. ID No. 5173 | 1367135 | 1367240 | 1367476 | Seq. ID No. 5174 |
| 2474b | Seq. ID No. 5175 | 1367135 | 1367189 | 1367476 | Seq. ID No. 5176 |
| 2474c | Seq. ID No. 5177 | 1367135 | 1367135 | 1367476 | Seq. ID No. 5178 |
| 2475a | Seq. ID No. 5179 | 1370572 | 1370467 | 1367687 | Seq. ID No. 5180 |
| 2475b | Seq. ID No. 5181 | 1370572 | 1370503 | 1367687 | Seq. ID No. 5182 |
| 2475c | Seq. ID No. 5183 | 1370572 | 1370509 | 1367687 | Seq. ID No. 5184 |
| 2477 | Seq. ID No. 5185 | 1371542 | 1371515 | 1370790 | Seq. ID No. 5186 |
| 2480 | Seq. ID No. 5187 | 1372374 | 1372365 | 1371577 | Seq. ID No. 5188 |
| 2481a | Seq. ID No. 5189 | 1371566 | 1371596 | 1372078 | Seq. ID No. 5190 |
| 2481b | Seq. ID No. 5191 | 1371566 | 1371569 | 1372078 | Seq. ID No. 5192 |
| 2482 | Seq. ID No. 5193 | 1372662 | 1372662 | 1372375 | Seq. ID No. 5194 |
| 2483 | Seq. ID No. 5195 | 1373156 | 1373144 | 1372683 | Seq. ID No. 5196 |
| 2484a | Seq. ID No. 5197 | 1373586 | 1373568 | 1373149 | Seq. ID No. 5198 |
| 2484b | Seq. ID No. 5199 | 1373586 | 1373580 | 1373149 | Seq. ID No. 5200 |
| 2485 | Seq. ID No. 5201 | 1374427 | 1374400 | 1373168 | Seq. ID No. 5202 |
| 2489 | Seq. ID No. 5203 | 1375804 | 1375792 | 1374428 | Seq. ID No. 5204 |
| 2490 | Seq. ID No. 5205 | 1376880 | 1376880 | 1375942 | Seq. ID No. 5206 |
| 2492a | Seq. ID No. 5207 | 1378075 | 1377256 | 1376897 | Seq. ID No. 5208 |
| 2492b | Seq. ID No. 5209 | 1378075 | 1378060 | 1376897 | Seq. ID No. 5210 |
| 2494 | Seq. ID No. 5211 | 1379477 | 1379453 | 1378050 | Seq. ID No. 5212 |
| 2495a | Seq. ID No. 5213 | 1380488 | 1380320 | 1379526 | Seq. ID No. 5214 |
| 2495b | Seq. ID No. 5215 | 1380488 | 1380479 | 1379526 | Seq. ID No. 5216 |
| 2497a | Seq. ID No. 5217 | 1382929 | 1382839 | 1380632 | Seq. ID No. 5218 |
| 2497b | Seq. ID No. 5219 | 1382929 | 1382926 | 1380632 | Seq. ID No. 5220 |
| 2498b | Seq. ID No. 5221 | 1380214 | 1380247 | 1380741 | Seq. ID No. 5222 |
| 2500a | Seq. ID No. 5223 | 1383330 | 1383216 | 1382926 | Seq. ID No. 5224 |
| 2500b | Seq. ID No. 5225 | 1383330 | 1383318 | 1382926 | Seq. ID No. 5226 |
| 2500c | Seq. ID No. 5227 | 1383330 | 1383327 | 1382926 | Seq. ID No. 5228 |
| 2501a | Seq. ID No. 5229 | 1384292 | 1384196 | 1383324 | Seq. ID No. 5230 |
| 2501b | Seq. ID No. 5231 | 1384292 | 1384280 | 1383324 | Seq. ID No. 5232 |
| 2504a | Seq. ID No. 5233 | 1384748 | 1384718 | 1384293 | Seq. ID No. 5234 |
| 2504b | Seq. ID No. 5235 | 1384748 | 1384724 | 1384293 | Seq. ID No. 5236 |
| 2505 | Seq. ID No. 5237 | 1385365 | 1385353 | 1384976 | Seq. ID No. 5238 |
| 2506 | Seq. ID No. 5239 | 1386567 | 1386534 | 1385977 | Seq. ID No. 5240 |
| 2507b | Seq. ID No. 5241 | 1387792 | 1387783 | 1386590 | Seq. ID No. 5242 |
| 2507c | Seq. ID No. 5243 | 1387792 | 1387792 | 1386590 | Seq. ID No. 5244 |
| 2510 | Seq. ID No. 5245 | 1388536 | 1388533 | 1387814 | Seq. ID No. 5246 |
| 2512c | Seq. ID No. 5247 | 1388904 | 1388901 | 1388533 | Seq. ID No. 5248 |
| 2513a | Seq. ID No. 5249 | 1389534 | 1389486 | 1388905 | Seq. ID No. 5250 |
| 2513b | Seq. ID No. 5251 | 1389534 | 1389516 | 1388905 | Seq. ID No. 5252 |
| 2513c | Seq. ID No. 5253 | 1389534 | 1389525 | 1388905 | Seq. ID No. 5254 |
| 2514 | Seq. ID No. 5255 | 1390141 | 1390135 | 1389491 | Seq. ID No. 5256 |
| 2515 | Seq. ID No. 5257 | 1390451 | 1390451 | 1390137 | Seq. ID No. 5258 |
| 2516a | Seq. ID No. 5259 | 1391642 | 1391396 | 1390482 | Seq. ID No. 5260 |
| 2516b | Seq. ID No. 5261 | 1391642 | 1391627 | 1390482 | Seq. ID No. 5262 |
| 2517a | Seq. ID No. 5263 | 1392251 | 1392185 | 1391643 | Seq. ID No. 5264 |
| 2517b | Seq. ID No. 5265 | 1392251 | 1392248 | 1391643 | Seq. ID No. 5266 |
| 2518a | Seq. ID No. 5267 | 1391575 | 1391770 | 1392045 | Seq. ID No. 5268 |
| 2519a | Seq. ID No. 5269 | 1392498 | 1392462 | 1392199 | Seq. ID No. 5270 |
| 2519b | Seq. ID No. 5271 | 1392498 | 1392492 | 1392199 | Seq. ID No. 5272 |
| 2520a | Seq. ID No. 5273 | 1394487 | 1394379 | 1392658 | Seq. ID No. 5274 |
| 2520b | Seq. ID No. 5275 | 1394487 | 1394400 | 1392658 | Seq. ID No. 5276 |
| 2520c | Seq. ID No. 5277 | 1394487 | 1394469 | 1392658 | Seq. ID No. 5278 |
| 2520d | Seq. ID No. 5279 | 1394487 | 1394487 | 1392658 | Seq. ID No. 5280 |
| 2521 | Seq. ID No. 5281 | 1393493 | 1393499 | 1393756 | Seq. ID No. 5282 |
| 2523 | Seq. ID No. 5283 | 1394650 | 1394647 | 1394381 | Seq. ID No. 5284 |
| 2525a | Seq. ID No. 5285 | 1396526 | 1396475 | 1396092 | Seq. ID No. 5286 |
| 2525b | Seq. ID No. 5287 | 1396526 | 1396490 | 1396092 | Seq. ID No. 5288 |
| 2527a | Seq. ID No. 5289 | 1394878 | 1395148 | 1397184 | Seq. ID No. 5290 |
| 2527b | Seq. ID No. 5291 | 1394878 | 1394953 | 1397184 | Seq. ID No. 5292 |
| 2528a | Seq. ID No. 5293 | 1399138 | 1398754 | 1397267 | Seq. ID No. 5294 |
| 2528b | Seq. ID No. 5295 | 1399138 | 1398850 | 1397267 | Seq. ID No. 5296 |
| 2528c | Seq. ID No. 5297 | 1399138 | 1398922 | 1397267 | Seq. ID No. 5298 |
| 2528d | Seq. ID No. 5299 | 1399138 | 1398961 | 1397267 | Seq. ID No. 5300 |
| 2532b | Seq. ID No. 5301 | 1397745 | 1397808 | 1398275 | Seq. ID No. 5302 |
| 2532c | Seq. ID No. 5303 | 1397745 | 1397772 | 1398275 | Seq. ID No. 5304 |
| 2534 | Seq. ID No. 5305 | 1399875 | 1399866 | 1398979 | Seq. ID No. 5306 |
| 2535a | Seq. ID No. 5307 | 1400680 | 1400668 | 1399847 | Seq. ID No. 5308 |
| 2535b | Seq. ID No. 5309 | 1400680 | 1400674 | 1399847 | Seq. ID No. 5310 |
| 2538 | Seq. ID No. 5311 | 1401349 | 1401310 | 1400681 | Seq. ID No. 5312 |
| 2541 | Seq. ID No. 5313 | 1401366 | 1401387 | 1401971 | Seq. ID No. 5314 |
| 2542a | Seq. ID No. 5315 | 1402014 | 1402296 | 1402676 | Seq. ID No. 5316 |
| 2542b | Seq. ID No. 5317 | 1402014 | 1402077 | 1402676 | Seq. ID No. 5318 |
| 2542c | Seq. ID No. 5319 | 1402014 | 1402062 | 1402676 | Seq. ID No. 5320 |
| 2543 | Seq. ID No. 5321 | 1403760 | 1403730 | 1403266 | Seq. ID No. 5322 |
| 2544 | Seq. ID No. 5323 | 1403203 | 1403254 | 1404255 | Seq. ID No. 5324 |
| 2545 | Seq. ID No. 5325 | 1404256 | 1404304 | 1404855 | Seq. ID No. 5326 |
| 2546 | Seq. ID No. 5327 | 1405305 | 1405302 | 1405021 | Seq. ID No. 5328 |
| 2547b | Seq. ID No. 5329 | 1406040 | 1406031 | 1405459 | Seq. ID No. 5330 |
| 2547c | Seq. ID No. 5331 | 1406040 | 1406037 | 1405459 | Seq. ID No. 5332 |
| 2548a | Seq. ID No. 5333 | 1404971 | 1405037 | 1405978 | Seq. ID No. 5334 |
| 2548b | Seq. ID No. 5335 | 1404971 | 1404995 | 1405978 | Seq. ID No. 5336 |
| 2549 | Seq. ID No. 5337 | 1405979 | 1405988 | 1406572 | Seq. ID No. 5338 |
| 2550b | Seq. ID No. 5339 | 1406454 | 1406559 | 1406936 | Seq. ID No. 5340 |
| 2550c | Seq. ID No. 5341 | 1406454 | 1406538 | 1406936 | Seq. ID No. 5342 |
| 2550e | Seq. ID No. 5343 | 1406454 | 1406469 | 1406936 | Seq. ID No. 5344 |
| 2552 | Seq. ID No. 5345 | 1407012 | 1407042 | 1408169 | Seq. ID No. 5346 |
| 2553 | Seq. ID No. 5347 | 1408922 | 1408922 | 1408764 | Seq. ID No. 5348 |
| 2554a | Seq. ID No. 5349 | 1410070 | 1410022 | 1409012 | Seq. ID No. 5350 |
| 2554b | Seq. ID No. 5351 | 1410070 | 1410031 | 1409012 | Seq. ID No. 5352 |
| 2555a | Seq. ID No. 5353 | 1410981 | 1410666 | 1410037 | Seq. ID No. 5354 |
| 2555b | Seq. ID No. 5355 | 1410981 | 1410930 | 1410037 | Seq. ID No. 5356 |
| 2555c | Seq. ID No. 5357 | 1410981 | 1410954 | 1410037 | Seq. ID No. 5358 |
| 2555d | Seq. ID No. 5359 | 1410981 | 1410960 | 1410037 | Seq. ID No. 5360 |
| 2558a | Seq. ID No. 5361 | 1411225 | 1411165 | 1410899 | Seq. ID No. 5362 |
| 2558b | Seq. ID No. 5363 | 1411225 | 1411183 | 1410899 | Seq. ID No. 5364 |
| 2559 | Seq. ID No. 5365 | 1411808 | 1411841 | 1413085 | Seq. ID No. 5366 |
| 2561 | Seq. ID No. 5367 | 1413552 | 1413567 | 1414415 | Seq. ID No. 5368 |
| 2562a | Seq. ID No. 5369 | 1414348 | 1414450 | 1414695 | Seq. ID No. 5370 |
| 2562c | Seq. ID No. 5371 | 1414348 | 1414396 | 1414695 | Seq. ID No. 5372 |
| 2563 | Seq. ID No. 5373 | 1416663 | 1416645 | 1414831 | Seq. ID No. 5374 |
| 2564a | Seq. ID No. 5375 | 1417575 | 1417422 | 1416748 | Seq. ID No. 5376 |
| 2564b | Seq. ID No. 5377 | 1417575 | 1417524 | 1416748 | Seq. ID No. 5378 |
| 2564c | Seq. ID No. 5379 | 1417575 | 1417533 | 1416748 | Seq. ID No. 5380 |
| 2564d | Seq. ID No. 5381 | 1417575 | 1417569 | 1416748 | Seq. ID No. 5382 |
| 2566 | Seq. ID No. 5383 | 1418729 | 1418708 | 1417590 | Seq. ID No. 5384 |
| 2567a | Seq. ID No. 5385 | 1419622 | 1419307 | 1418843 | Seq. ID No. 5386 |
| 2567b | Seq. ID No. 5387 | 1419622 | 1419610 | 1418843 | Seq. ID No. 5388 |
| 2568 | Seq. ID No. 5389 | 1420782 | 1420740 | 1419607 | Seq. ID No. 5390 |
| 2572 | Seq. ID No. 5391 | 1421908 | 1421875 | 1420760 | Seq. ID No. 5392 |
| 2573 | Seq. ID No. 5393 | 1422663 | 1422660 | 1421995 | Seq. ID No. 5394 |
| 2575 | Seq. ID No. 5395 | 1423311 | 1423263 | 1422889 | Seq. ID No. 5396 |
| 2576 | Seq. ID No. 5397 | 1424366 | 1424318 | 1423299 | Seq. ID No. 5398 |
| 2578 | Seq. ID No. 5399 | 1424941 | 1424926 | 1424564 | Seq. ID No. 5400 |
| 2579b | Seq. ID No. 5401 | 1425765 | 1425729 | 1425178 | Seq. ID No. 5402 |
| 2581 | Seq. ID No. 5403 | 1426786 | 1426756 | 1425986 | Seq. ID No. 5404 |
| 2582a | Seq. ID No. 5405 | 1427718 | 1427607 | 1426879 | Seq. ID No. 5406 |
| 2582b | Seq. ID No. 5407 | 1427718 | 1427694 | 1426879 | Seq. ID No. 5408 |
| 2582c | Seq. ID No. 5409 | 1427718 | 1427712 | 1426879 | Seq. ID No. 5410 |
| 2585 | Seq. ID No. 5411 | 1429438 | 1429423 | 1427699 | Seq. ID No. 5412 |
| 2587a | Seq. ID No. 5413 | 1429982 | 1429652 | 1429425 | Seq. ID No. 5414 |
| 2587b | Seq. ID No. 5415 | 1429982 | 1429982 | 1429425 | Seq. ID No. 5416 |
| 2588 | Seq. ID No. 5417 | 1430858 | 1430852 | 1429983 | Seq. ID No. 5418 |
| 2589 | Seq. ID No. 5419 | 1433411 | 1433399 | 1431450 | Seq. ID No. 5420 |
| 2591 | Seq. ID No. 5421 | 1434679 | 1434667 | 1433714 | Seq. ID No. 5422 |
| 2592 | Seq. ID No. 5423 | 1435611 | 1435470 | 1434667 | Seq. ID No. 5424 |
| 2593a | Seq. ID No. 5425 | 1436075 | 1435949 | 1435482 | Seq. ID No. 5426 |
| 2593b | Seq. ID No. 5427 | 1436075 | 1436042 | 1435482 | Seq. ID No. 5428 |
| 2594a | Seq. ID No. 5429 | 1436529 | 1436517 | 1436059 | Seq. ID No. 5430 |
| 2594b | Seq. ID No. 5431 | 1436529 | 1436526 | 1436059 | Seq. ID No. 5432 |
| 2596 | Seq. ID No. 5433 | 1437374 | 1437374 | 1436709 | Seq. ID No. 5434 |
| 2597a | Seq. ID No. 5435 | 1438188 | 1437930 | 1437334 | Seq. ID No. 5436 |
| 2597b | Seq. ID No. 5437 | 1438188 | 1438107 | 1437334 | Seq. ID No. 5438 |
| 2597c | Seq. ID No. 5439 | 1438188 | 1438158 | 1437334 | Seq. ID No. 5440 |
| 2598 | Seq. ID No. 5441 | 1440874 | 1440859 | 1438196 | Seq. ID No. 5442 |
| 2602 | Seq. ID No. 5443 | 1442399 | 1442396 | 1441065 | Seq. ID No. 5444 |
| 2605a | Seq. ID No. 5445 | 1442989 | 1442629 | 1442468 | Seq. ID No. 5446 |
| 2605b | Seq. ID No. 5447 | 1442989 | 1442968 | 1442468 | Seq. ID No. 5448 |
| 2606 | Seq. ID No. 5449 | 1443636 | 1443591 | 1442983 | Seq. ID No. 5450 |
| 2608 | Seq. ID No. 5451 | 1444685 | 1444676 | 1443603 | Seq. ID No. 5452 |
| 2610 | Seq. ID No. 5453 | 1444840 | 1444840 | 1445172 | Seq. ID No. 5454 |
| 2611 | Seq. ID No. 5455 | 1445942 | 1445927 | 1445280 | Seq. ID No. 5456 |
| 2613 | Seq. ID No. 5457 | 1446810 | 1446789 | 1445992 | Seq. ID No. 5458 |
| 2616a | Seq. ID No. 5459 | 1448123 | 1447217 | 1446882 | Seq. ID No. 5460 |
| 2616b | Seq. ID No. 5461 | 1448123 | 1447583 | 1446882 | Seq. ID No. 5462 |
| 2616c | Seq. ID No. 5463 | 1448123 | 1447613 | 1446882 | Seq. ID No. 5464 |
| 2616d | Seq. ID No. 5465 | 1448123 | 1447805 | 1446882 | Seq. ID No. 5466 |
| 2616e | Seq. ID No. 5467 | 1448123 | 1447850 | 1446882 | Seq. ID No. 5468 |
| 2616f | Seq. ID No. 5469 | 1448123 | 1447976 | 1446882 | Seq. ID No. 5470 |
| 2616g | Seq. ID No. 5471 | 1448123 | 1448084 | 1446882 | Seq. ID No. 5472 |
| 2616h | Seq. ID No. 5473 | 1448123 | 1448093 | 1446882 | Seq. ID No. 5474 |
| 2617a | Seq. ID No. 5475 | 1448922 | 1448838 | 1448077 | Seq. ID No. 5476 |
| 2617b | Seq. ID No. 5477 | 1448922 | 1448922 | 1448077 | Seq. ID No. 5478 |
| 2618a | Seq. ID No. 5479 | 1448201 | 1448282 | 1448533 | Seq. ID No. 5480 |
| 2618b | Seq. ID No. 5481 | 1448201 | 1448249 | 1448533 | Seq. ID No. 5482 |
| 2619 | Seq. ID No. 5483 | 1448954 | 1448987 | 1449424 | Seq. ID No. 5484 |
| 2620 | Seq. ID No. 5485 | 1449683 | 1449668 | 1449456 | Seq. ID No. 5486 |
| 2621a | Seq. ID No. 5487 | 1449261 | 1449426 | 1449758 | Seq. ID No. 5488 |
| 2623a | Seq. ID No. 5489 | 1451028 | 1450824 | 1449895 | Seq. ID No. 5490 |
| 2623b | Seq. ID No. 5491 | 1451028 | 1451010 | 1449895 | Seq. ID No. 5492 |
| 2624a | Seq. ID No. 5493 | 1452149 | 1452101 | 1451130 | Seq. ID No. 5494 |
| 2624b | Seq. ID No. 5495 | 1452149 | 1452110 | 1451130 | Seq. ID No. 5496 |
| 2625a | Seq. ID No. 5497 | 1454863 | 1453486 | 1452116 | Seq. ID No. 5498 |
| 2625b | Seq. ID No. 5499 | 1454863 | 1454809 | 1452116 | Seq. ID No. 5500 |
| 2626a | Seq. ID No. 5501 | 1456055 | 1455935 | 1454820 | Seq. ID No. 5502 |
| 2626b | Seq. ID No. 5503 | 1456055 | 1455998 | 1454820 | Seq. ID No. 5504 |
| 2627a | Seq. ID No. 5505 | 1456504 | 1456453 | 1456079 | Seq. ID No. 5506 |
| 2627b | Seq. ID No. 5507 | 1456504 | 1456465 | 1456079 | Seq. ID No. 5508 |
| 2628 | Seq. ID No. 5509 | 1457511 | 1457484 | 1456576 | Seq. ID No. 5510 |
| 2629a | Seq. ID No. 5511 | 1459560 | 1459494 | 1457665 | Seq. ID No. 5512 |
| 2629b | Seq. ID No. 5513 | 1459560 | 1459539 | 1457665 | Seq. ID No. 5514 |
| 2631 | Seq. ID No. 5515 | 1458482 | 1458482 | 1458249 | Seq. ID No. 5516 |
| 2632 | Seq. ID No. 5517 | 1459307 | 1459322 | 1459567 | Seq. ID No. 5518 |
| 2633a | Seq. ID No. 5519 | 1461946 | 1461220 | 1459916 | Seq. ID No. 5520 |
| 2633b | Seq. ID No. 5521 | 1461946 | 1461544 | 1459916 | Seq. ID No. 5522 |
| 2633c | Seq. ID No. 5523 | 1461946 | 1461727 | 1459916 | Seq. ID No. 5524 |
| 2633d | Seq. ID No. 5525 | 1461946 | 1461778 | 1459916 | Seq. ID No. 5526 |
| 2633e | Seq. ID No. 5527 | 1461946 | 1461904 | 1459916 | Seq. ID No. 5528 |
| 2637a | Seq. ID No. 5529 | 1463997 | 1463967 | 1462111 | Seq. ID No. 5530 |
| 2637b | Seq. ID No. 5531 | 1463997 | 1463991 | 1462111 | Seq. ID No. 5532 |
| 2642 | Seq. ID No. 5533 | 1463789 | 1463825 | 1464121 | Seq. ID No. 5534 |
| 2643a | Seq. ID No. 5535 | 1466534 | 1466318 | 1464372 | Seq. ID No. 5536 |
| 2643b | Seq. ID No. 5537 | 1466534 | 1466522 | 1464372 | Seq. ID No. 5538 |
| 2645 | Seq. ID No. 5539 | 1467800 | 1467782 | 1466751 | Seq. ID No. 5540 |
| 2647 | Seq. ID No. 5541 | 1468855 | 1468774 | 1467806 | Seq. ID No. 5542 |
| 2648a | Seq. ID No. 5543 | 1468422 | 1468452 | 1468748 | Seq. ID No. 5544 |
| 2648b | Seq. ID No. 5545 | 1468422 | 1468449 | 1468748 | Seq. ID No. 5546 |
| 2649 | Seq. ID No. 5547 | 1470087 | 1470087 | 1468786 | Seq. ID No. 5548 |
| 2650 | Seq. ID No. 5549 | 1468988 | 1468994 | 1469452 | Seq. ID No. 5550 |
| 2652a | Seq. ID No. 5551 | 1471356 | 1471212 | 1470088 | Seq. ID No. 5552 |
| 2652b | Seq. ID No. 5553 | 1471356 | 1471272 | 1470088 | Seq. ID No. 5554 |
| 2655a | Seq. ID No. 5555 | 1471693 | 1471618 | 1471307 | Seq. ID No. 5556 |
| 2655b | Seq. ID No. 5557 | 1471693 | 1471666 | 1471307 | Seq. ID No. 5558 |
| 2656 | Seq. ID No. 5559 | 1472777 | 1472771 | 1471758 | Seq. ID No. 5560 |
| 2658a | Seq. ID No. 5561 | 1473451 | 1473403 | 1472798 | Seq. ID No. 5562 |
| 2658b | Seq. ID No. 5563 | 1473451 | 1473430 | 1472798 | Seq. ID No. 5564 |
| 2659a | Seq. ID No. 5565 | 1474937 | 1474799 | 1473552 | Seq. ID No. 5566 |
| 2659b | Seq. ID No. 5567 | 1474937 | 1474931 | 1473552 | Seq. ID No. 5568 |
| 2660 | Seq. ID No. 5569 | 1474156 | 1474156 | 1474392 | Seq. ID No. 5570 |
| 2662 | Seq. ID No. 5571 | 1475396 | 1475390 | 1474959 | Seq. ID No. 5572 |
| 2664a | Seq. ID No. 5573 | 1477384 | 1477357 | 1475402 | Seq. ID No. 5574 |
| 2664b | Seq. ID No. 5575 | 1477384 | 1477375 | 1475402 | Seq. ID No. 5576 |
| 2665 | Seq. ID No. 5577 | 1476161 | 1476173 | 1476403 | Seq. ID No. 5578 |
| 2666a | Seq. ID No. 5579 | 1479987 | 1479798 | 1477390 | Seq. ID No. 5580 |
| 2666b | Seq. ID No. 5581 | 1479987 | 1479987 | 1477390 | Seq. ID No. 5582 |
| 2667a | Seq. ID No. 5583 | 1480983 | 1480833 | 1480003 | Seq. ID No. 5584 |
| 2667b | Seq. ID No. 5585 | 1480983 | 1480860 | 1480003 | Seq. ID No. 5586 |
| 2669a | Seq. ID No. 5587 | 1482332 | 1482248 | 1480950 | Seq. ID No. 5588 |
| 2669b | Seq. ID No. 5589 | 1482332 | 1482296 | 1480950 | Seq. ID No. 5590 |
| 2669c | Seq. ID No. 5591 | 1482332 | 1482305 | 1480950 | Seq. ID No. 5592 |
| 2672 | Seq. ID No. 5593 | 1481077 | 1481095 | 1481394 | Seq. ID No. 5594 |
| 2673 | Seq. ID No. 5595 | 1481941 | 1481944 | 1482171 | Seq. ID No. 5596 |
| 2674a | Seq. ID No. 5597 | 1484170 | 1483174 | 1482620 | Seq. ID No. 5598 |
| 2674b | Seq. ID No. 5599 | 1484170 | 1484128 | 1482620 | Seq. ID No. 5600 |
| 2676 | Seq. ID No. 5601 | 1485021 | 1485003 | 1484260 | Seq. ID No. 5602 |
| 2677a | Seq. ID No. 5603 | 1486187 | 1486013 | 1485009 | Seq. ID No. 5604 |
| 2677b | Seq. ID No. 5605 | 1486187 | 1486094 | 1485009 | Seq. ID No. 5606 |
| 2677c | Seq. ID No. 5607 | 1486187 | 1486172 | 1485009 | Seq. ID No. 5608 |
| 2679a | Seq. ID No. 5609 | 1486773 | 1486521 | 1486042 | Seq. ID No. 5610 |
| 2679b | Seq. ID No. 5611 | 1486773 | 1486701 | 1486042 | Seq. ID No. 5612 |
| 2679c | Seq. ID No. 5613 | 1486773 | 1486764 | 1486042 | Seq. ID No. 5614 |
| 2679d | Seq. ID No. 5615 | 1486773 | 1486773 | 1486042 | Seq. ID No. 5616 |
| 2680a | Seq. ID No. 5617 | 1488572 | 1488065 | 1487013 | Seq. ID No. 5618 |
| 2680b | Seq. ID No. 5619 | 1488572 | 1488401 | 1487013 | Seq. ID No. 5620 |
| 2680c | Seq. ID No. 5621 | 1488572 | 1488572 | 1487013 | Seq. ID No. 5622 |
| 2683a | Seq. ID No. 5623 | 1487806 | 1487878 | 1488216 | Seq. ID No. 5624 |
| 2683b | Seq. ID No. 5625 | 1487806 | 1487839 | 1488216 | Seq. ID No. 5626 |
| 2684a | Seq. ID No. 5627 | 1489943 | 1489898 | 1488828 | Seq. ID No. 5628 |
| 2684b | Seq. ID No. 5629 | 1489943 | 1489913 | 1488828 | Seq. ID No. 5630 |
| 2686 | Seq. ID No. 5631 | 1491201 | 1491189 | 1489990 | Seq. ID No. 5632 |
| 2688a | Seq. ID No. 5633 | 1491962 | 1491728 | 1491381 | Seq. ID No. 5634 |
| 2688b | Seq. ID No. 5635 | 1491962 | 1491923 | 1491381 | Seq. ID No. 5636 |
| 2688c | Seq. ID No. 5637 | 1491962 | 1491962 | 1491381 | Seq. ID No. 5638 |
| 2689a | Seq. ID No. 5639 | 1491463 | 1491526 | 1491783 | Seq. ID No. 5640 |
| 2689b | Seq. ID No. 5641 | 1491463 | 1491508 | 1491783 | Seq. ID No. 5642 |
| 2690a | Seq. ID No. 5643 | 1492662 | 1492314 | 1492012 | Seq. ID No. 5644 |
| 2690b | Seq. ID No. 5645 | 1492662 | 1492623 | 1492012 | Seq. ID No. 5646 |
| 2690c | Seq. ID No. 5647 | 1492662 | 1492656 | 1492012 | Seq. ID No. 5648 |
| 2692b | Seq. ID No. 5649 | 1492270 | 1492330 | 1492617 | Seq. ID No. 5650 |
| 2693 | Seq. ID No. 5651 | 1494018 | 1493982 | 1492696 | Seq. ID No. 5652 |
| 2694a | Seq. ID No. 5653 | 1495259 | 1494968 | 1493979 | Seq. ID No. 5654 |
| 2694b | Seq. ID No. 5655 | 1495259 | 1495259 | 1493979 | Seq. ID No. 5656 |
| 2695 | Seq. ID No. 5657 | 1496442 | 1496442 | 1495243 | Seq. ID No. 5658 |
| 2696a | Seq. ID No. 5659 | 1496578 | 1496656 | 1497375 | Seq. ID No. 5660 |
| 2696b | Seq. ID No. 5661 | 1496578 | 1496623 | 1497375 | Seq. ID No. 5662 |
| 2705 | Seq. ID No. 5663 | 1501429 | 1501429 | 1501343 | Seq. ID No. 5664 |
| 2709 | Seq. ID No. 5665 | 1505327 | 1505300 | 1504359 | Seq. ID No. 5666 |
| 2710c | Seq. ID No. 5667 | 1504984 | 1504885 | 1504604 | Seq. ID No. 5668 |
| 2712 | Seq. ID No. 5669 | 1506916 | 1506898 | 1505546 | Seq. ID No. 5670 |
| 2713 | Seq. ID No. 5671 | 1507318 | 1507309 | 1506926 | Seq. ID No. 5672 |
| 2714 | Seq. ID No. 5673 | 1507705 | 1507696 | 1507319 | Seq. ID No. 5674 |
| 2716a | Seq. ID No. 5675 | 1508501 | 1508477 | 1507878 | Seq. ID No. 5676 |
| 2716b | Seq. ID No. 5677 | 1508501 | 1508486 | 1507878 | Seq. ID No. 5678 |
| 2716c | Seq. ID No. 5679 | 1508501 | 1508501 | 1507878 | Seq. ID No. 5680 |
| 2718a | Seq. ID No. 5681 | 1508621 | 1508753 | 1509109 | Seq. ID No. 5682 |
| 2718b | Seq. ID No. 5683 | 1508621 | 1508702 | 1509109 | Seq. ID No. 5684 |
| 2718c | Seq. ID No. 5685 | 1508621 | 1508624 | 1509109 | Seq. ID No. 5686 |
| 2719a | Seq. ID No. 5687 | 1509372 | 1509399 | 1509650 | Seq. ID No. 5688 |
| 2719b | Seq. ID No. 5689 | 1509372 | 1509378 | 1509650 | Seq. ID No. 5690 |
| 2721a | Seq. ID No. 5691 | 1509110 | 1509434 | 1510138 | Seq. ID No. 5692 |
| 2721b | Seq. ID No. 5693 | 1509110 | 1509143 | 1510138 | Seq. ID No. 5694 |
| 2722a | Seq. ID No. 5695 | 1510153 | 1510180 | 1510692 | Seq. ID No. 5696 |
| 2722b | Seq. ID No. 5697 | 1510153 | 1510162 | 1510692 | Seq. ID No. 5698 |
| 2723 | Seq. ID No. 5699 | 1511400 | 1511397 | 1510747 | Seq. ID No. 5700 |
| 2724 | Seq. ID No. 5701 | 1512181 | 1512169 | 1511390 | Seq. ID No. 5702 |
| 2725a | Seq. ID No. 5703 | 1511541 | 1511553 | 1511786 | Seq. ID No. 5704 |
| 2725b | Seq. ID No. 5705 | 1511541 | 1511541 | 1511786 | Seq. ID No. 5706 |
| 2726 | Seq. ID No. 5707 | 1512847 | 1512844 | 1512182 | Seq. ID No. 5708 |
| 2728a | Seq. ID No. 5709 | 1512137 | 1512284 | 1512532 | Seq. ID No. 5710 |
| 2728b | Seq. ID No. 5711 | 1512137 | 1512275 | 1512532 | Seq. ID No. 5712 |
| 2731a | Seq. ID No. 5713 | 1512977 | 1513013 | 1513606 | Seq. ID No. 5714 |
| 2731b | Seq. ID No. 5715 | 1512977 | 1512980 | 1513606 | Seq. ID No. 5716 |
| 2732 | Seq. ID No. 5717 | 1514469 | 1514454 | 1513648 | Seq. ID No. 5718 |
| 2734a | Seq. ID No. 5719 | 1514794 | 1515100 | 1515729 | Seq. ID No. 5720 |
| 2734b | Seq. ID No. 5721 | 1514794 | 1514950 | 1515729 | Seq. ID No. 5722 |
| 2734c | Seq. ID No. 5723 | 1514794 | 1514827 | 1515729 | Seq. ID No. 5724 |
| 2735a | Seq. ID No. 5725 | 1516826 | 1516781 | 1515786 | Seq. ID No. 5726 |
| 2735b | Seq. ID No. 5727 | 1516826 | 1516790 | 1515786 | Seq. ID No. 5728 |
| 2738a | Seq. ID No. 5729 | 1518180 | 1517928 | 1516897 | Seq. ID No. 5730 |
| 2738b | Seq. ID No. 5731 | 1518180 | 1518084 | 1516897 | Seq. ID No. 5732 |
| 2738c | Seq. ID No. 5733 | 1518180 | 1518165 | 1516897 | Seq. ID No. 5734 |
| 2738d | Seq. ID No. 5735 | 1518180 | 1518177 | 1516897 | Seq. ID No. 5736 |
| 2742a | Seq. ID No. 5737 | 1518180 | 1518327 | 1518965 | Seq. ID No. 5738 |
| 2742b | Seq. ID No. 5739 | 1518180 | 1518195 | 1518965 | Seq. ID No. 5740 |
| 2743a | Seq. ID No. 5741 | 1520031 | 1520013 | 1519519 | Seq. ID No. 5742 |
| 2743b | Seq. ID No. 5743 | 1520031 | 1520022 | 1519519 | Seq. ID No. 5744 |
| 2744 | Seq. ID No. 5745 | 1520872 | 1520860 | 1520513 | Seq. ID No. 5746 |
| 2745a | Seq. ID No. 5747 | 1519154 | 1519358 | 1520725 | Seq. ID No. 5748 |
| 2745b | Seq. ID No. 5749 | 1519154 | 1519169 | 1520725 | Seq. ID No. 5750 |
| 2746 | Seq. ID No. 5751 | 1520820 | 1520832 | 1521431 | Seq. ID No. 5752 |
| 2747a | Seq. ID No. 5753 | 1522209 | 1522173 | 1521472 | Seq. ID No. 5754 |
| 2747b | Seq. ID No. 5755 | 1522209 | 1522209 | 1521472 | Seq. ID No. 5756 |
| 2748a | Seq. ID No. 5757 | 1523583 | 1523526 | 1522213 | Seq. ID No. 5758 |
| 2748b | Seq. ID No. 5759 | 1523583 | 1523568 | 1522213 | Seq. ID No. 5760 |
| 2748c | Seq. ID No. 5761 | 1523583 | 1523571 | 1522213 | Seq. ID No. 5762 |
| 2749a | Seq. ID No. 5763 | 1524727 | 1523995 | 1523705 | Seq. ID No. 5764 |
| 2749b | Seq. ID No. 5765 | 1524727 | 1524178 | 1523705 | Seq. ID No. 5766 |
| 2749c | Seq. ID No. 5767 | 1524727 | 1524541 | 1523705 | Seq. ID No. 5768 |
| 2749d | Seq. ID No. 5769 | 1524727 | 1524631 | 1523705 | Seq. ID No. 5770 |
| 2749e | Seq. ID No. 5771 | 1524727 | 1524721 | 1523705 | Seq. ID No. 5772 |
| 2750a | Seq. ID No. 5773 | 1525364 | 1525289 | 1524954 | Seq. ID No. 5774 |
| 2750b | Seq. ID No. 5775 | 1525364 | 1525319 | 1524954 | Seq. ID No. 5776 |
| 2752 | Seq. ID No. 5777 | 1525678 | 1525690 | 1527060 | Seq. ID No. 5778 |
| 2753 | Seq. ID No. 5779 | 1528083 | 1528029 | 1527202 | Seq. ID No. 5780 |
| 2757a | Seq. ID No. 5781 | 1528638 | 1528524 | 1528156 | Seq. ID No. 5782 |
| 2757b | Seq. ID No. 5783 | 1528638 | 1528623 | 1528156 | Seq. ID No. 5784 |
| 2758a | Seq. ID No. 5785 | 1529611 | 1529440 | 1528613 | Seq. ID No. 5786 |
| 2758b | Seq. ID No. 5787 | 1529611 | 1529485 | 1528613 | Seq. ID No. 5788 |
| 2758c | Seq. ID No. 5789 | 1529611 | 1529497 | 1528613 | Seq. ID No. 5790 |
| 2758d | Seq. ID No. 5791 | 1529611 | 1529581 | 1528613 | Seq. ID No. 5792 |
| 2759a | Seq. ID No. 5793 | 1530134 | 1530053 | 1529427 | Seq. ID No. 5794 |
| 2759b | Seq. ID No. 5795 | 1530134 | 1530134 | 1529427 | Seq. ID No. 5796 |
| 2760a | Seq. ID No. 5797 | 1530993 | 1530369 | 1530127 | Seq. ID No. 5798 |
| 2760b | Seq. ID No. 5799 | 1530993 | 1530963 | 1530127 | Seq. ID No. 5800 |
| 2760c | Seq. ID No. 5801 | 1530993 | 1530987 | 1530127 | Seq. ID No. 5802 |
| 2762 | Seq. ID No. 5803 | 1531820 | 1531820 | 1531293 | Seq. ID No. 5804 |
| 2763a | Seq. ID No. 5805 | 1532723 | 1532576 | 1531821 | Seq. ID No. 5806 |
| 2763b | Seq. ID No. 5807 | 1532723 | 1532588 | 1531821 | Seq. ID No. 5808 |
| 2763c | Seq. ID No. 5809 | 1532723 | 1532666 | 1531821 | Seq. ID No. 5810 |
| 2764 | Seq. ID No. 5811 | 1533169 | 1533145 | 1532774 | Seq. ID No. 5812 |
| 2765b | Seq. ID No. 5813 | 1533695 | 1533587 | 1533045 | Seq. ID No. 5814 |
| 2765c | Seq. ID No. 5815 | 1533695 | 1533623 | 1533045 | Seq. ID No. 5816 |
| 2766a | Seq. ID No. 5817 | 1535067 | 1535031 | 1533682 | Seq. ID No. 5818 |
| 2766b | Seq. ID No. 5819 | 1535067 | 1535061 | 1533682 | Seq. ID No. 5820 |
| 2768a | Seq. ID No. 5821 | 1536982 | 1536859 | 1535072 | Seq. ID No. 5822 |
| 2768b | Seq. ID No. 5823 | 1536982 | 1536934 | 1535072 | Seq. ID No. 5824 |
| 2768c | Seq. ID No. 5825 | 1536982 | 1536958 | 1535072 | Seq. ID No. 5826 |
| 2769a | Seq. ID No. 5827 | 1535138 | 1535258 | 1535506 | Seq. ID No. 5828 |
| 2770 | Seq. ID No. 5829 | 1536567 | 1536576 | 1536812 | Seq. ID No. 5830 |
| 2771a | Seq. ID No. 5831 | 1537982 | 1537820 | 1536999 | Seq. ID No. 5832 |
| 2771b | Seq. ID No. 5833 | 1537982 | 1537907 | 1536999 | Seq. ID No. 5834 |
| 2773 | Seq. ID No. 5835 | 1539371 | 1539353 | 1538247 | Seq. ID No. 5836 |
| 2774a | Seq. ID No. 5837 | 1540719 | 1540500 | 1539532 | Seq. ID No. 5838 |
| 2774b | Seq. ID No. 5839 | 1540719 | 1540638 | 1539532 | Seq. ID No. 5840 |
| 2774c | Seq. ID No. 5841 | 1540719 | 1540674 | 1539532 | Seq. ID No. 5842 |
| 2776a | Seq. ID No. 5843 | 1541302 | 1541248 | 1540820 | Seq. ID No. 5844 |
| 2776b | Seq. ID No. 5845 | 1541302 | 1541293 | 1540820 | Seq. ID No. 5846 |
| 2777 | Seq. ID No. 5847 | 1542467 | 1542467 | 1541280 | Seq. ID No. 5848 |
| 2778 | Seq. ID No. 5849 | 1543318 | 1543297 | 1542479 | Seq. ID No. 5850 |
| 2779 | Seq. ID No. 5851 | 1544480 | 1544465 | 1543347 | Seq. ID No. 5852 |
| 2780 | Seq. ID No. 5853 | 1545002 | 1545002 | 1544664 | Seq. ID No. 5854 |
| 2784 | Seq. ID No. 5855 | 1545859 | 1545892 | 1547058 | Seq. ID No. 5856 |
| 2785a | Seq. ID No. 5857 | 1548736 | 1548439 | 1547480 | Seq. ID No. 5858 |
| 2785b | Seq. ID No. 5859 | 1548736 | 1548700 | 1547480 | Seq. ID No. 5860 |
| 2786a | Seq. ID No. 5861 | 1549210 | 1549273 | 1549941 | Seq. ID No. 5862 |
| 2786b | Seq. ID No. 5863 | 1549210 | 1549219 | 1549941 | Seq. ID No. 5864 |
| 2786c | Seq. ID No. 5865 | 1549210 | 1549210 | 1549941 | Seq. ID No. 5866 |
| 2787a | Seq. ID No. 5867 | 1550972 | 1550930 | 1550007 | Seq. ID No. 5868 |
| 2787b | Seq. ID No. 5869 | 1550972 | 1550948 | 1550007 | Seq. ID No. 5870 |
| 2787c | Seq. ID No. 5871 | 1550972 | 1550969 | 1550007 | Seq. ID No. 5872 |
| 2788b | Seq. ID No. 5873 | 1550131 | 1550137 | 1550595 | Seq. ID No. 5874 |
| 2789 | Seq. ID No. 5875 | 1552131 | 1552116 | 1550923 | Seq. ID No. 5876 |
| 2790 | Seq. ID No. 5877 | 1552550 | 1552550 | 1552113 | Seq. ID No. 5878 |
| 2791 | Seq. ID No. 5879 | 1552345 | 1552345 | 1552118 | Seq. ID No. 5880 |
| 2793 | Seq. ID No. 5881 | 1553495 | 1553474 | 1552551 | Seq. ID No. 5882 |
| 2794 | Seq. ID No. 5883 | 1553299 | 1553296 | 1553060 | Seq. ID No. 5884 |
| 2796a | Seq. ID No. 5885 | 1554180 | 1554075 | 1553785 | Seq. ID No. 5886 |
| 2798 | Seq. ID No. 5887 | 1554325 | 1554301 | 1553969 | Seq. ID No. 5888 |
| 2799a | Seq. ID No. 5889 | 1553810 | 1553918 | 1554184 | Seq. ID No. 5890 |
| 2799b | Seq. ID No. 5891 | 1553810 | 1553822 | 1554184 | Seq. ID No. 5892 |
| 2801a | Seq. ID No. 5893 | 1556543 | 1556078 | 1555158 | Seq. ID No. 5894 |
| 2801b | Seq. ID No. 5895 | 1556543 | 1556471 | 1555158 | Seq. ID No. 5896 |
| 2804 | Seq. ID No. 5897 | 1557136 | 1557124 | 1556468 | Seq. ID No. 5898 |
| 2805a | Seq. ID No. 5899 | 1558765 | 1557724 | 1557347 | Seq. ID No. 5900 |
| 2805b | Seq. ID No. 5901 | 1558765 | 1558723 | 1557347 | Seq. ID No. 5902 |
| 2809 | Seq. ID No. 5903 | 1559388 | 1559373 | 1558726 | Seq. ID No. 5904 |
| 2810a | Seq. ID No. 5905 | 1560847 | 1559776 | 1559384 | Seq. ID No. 5906 |
| 2810b | Seq. ID No. 5907 | 1560847 | 1560571 | 1559384 | Seq. ID No. 5908 |
| 2810c | Seq. ID No. 5909 | 1560847 | 1560772 | 1559384 | Seq. ID No. 5910 |
| 2810d | Seq. ID No. 5911 | 1560847 | 1560826 | 1559384 | Seq. ID No. 5912 |
| 2812 | Seq. ID No. 5913 | 1562885 | 1562828 | 1561227 | Seq. ID No. 5914 |
| 2814 | Seq. ID No. 5915 | 1563915 | 1563912 | 1563031 | Seq. ID No. 5916 |
| 2815 | Seq. ID No. 5917 | 1564857 | 1564806 | 1563925 | Seq. ID No. 5918 |
| 2816 | Seq. ID No. 5919 | 1565582 | 1565555 | 1564821 | Seq. ID No. 5920 |
| 2818a | Seq. ID No. 5921 | 1567335 | 1565871 | 1565566 | Seq. ID No. 5922 |
| 2818b | Seq. ID No. 5923 | 1567335 | 1566276 | 1565566 | Seq. ID No. 5924 |
| 2818c | Seq. ID No. 5925 | 1567335 | 1567161 | 1565566 | Seq. ID No. 5926 |
| 2818d | Seq. ID No. 5927 | 1567335 | 1567281 | 1565566 | Seq. ID No. 5928 |
| 2818e | Seq. ID No. 5929 | 1567335 | 1567335 | 1565566 | Seq. ID No. 5930 |
| 2819 | Seq. ID No. 5931 | 1568219 | 1568195 | 1567689 | Seq. ID No. 5932 |
| 2820 | Seq. ID No. 5933 | 1569072 | 1569063 | 1568275 | Seq. ID No. 5934 |
| 2821a | Seq. ID No. 5935 | 1570005 | 1569981 | 1569271 | Seq. ID No. 5936 |
| 2821b | Seq. ID No. 5937 | 1570005 | 1570002 | 1569271 | Seq. ID No. 5938 |
| 2822a | Seq. ID No. 5939 | 1571393 | 1570382 | 1570002 | Seq. ID No. 5940 |
| 2822b | Seq. ID No. 5941 | 1571393 | 1571372 | 1570002 | Seq. ID No. 5942 |
| 2823 | Seq. ID No. 5943 | 1571696 | 1571675 | 1571394 | Seq. ID No. 5944 |
| 2824a | Seq. ID No. 5945 | 1573753 | 1573744 | 1571678 | Seq. ID No. 5946 |
| 2824b | Seq. ID No. 5947 | 1573753 | 1573753 | 1571678 | Seq. ID No. 5948 |
| 2829a | Seq. ID No. 5949 | 1574248 | 1574341 | 1574763 | Seq. ID No. 5950 |
| 2829b | Seq. ID No. 5951 | 1574248 | 1574260 | 1574763 | Seq. ID No. 5952 |
| 2831b | Seq. ID No. 5953 | 1574873 | 1574897 | 1575280 | Seq. ID No. 5954 |
| 2832 | Seq. ID No. 5955 | 1575871 | 1575832 | 1575464 | Seq. ID No. 5956 |
| 2833 | Seq. ID No. 5957 | 1576608 | 1576584 | 1576153 | Seq. ID No. 5958 |
| 2834a | Seq. ID No. 5959 | 1580917 | 1580884 | 1576613 | Seq. ID No. 5960 |
| 2834b | Seq. ID No. 5961 | 1580917 | 1580917 | 1576613 | Seq. ID No. 5962 |
| 2837a | Seq. ID No. 5963 | 1578356 | 1578371 | 1578688 | Seq. ID No. 5964 |
| 2837b | Seq. ID No. 5965 | 1578356 | 1578356 | 1578688 | Seq. ID No. 5966 |
| 2842a | Seq. ID No. 5967 | 1581713 | 1581578 | 1580898 | Seq. ID No. 5968 |
| 2842b | Seq. ID No. 5969 | 1581713 | 1581665 | 1580898 | Seq. ID No. 5970 |
| 2843a | Seq. ID No. 5971 | 1583065 | 1583050 | 1581806 | Seq. ID No. 5972 |
| 2843b | Seq. ID No. 5973 | 1583065 | 1583062 | 1581806 | Seq. ID No. 5974 |
| 2845 | Seq. ID No. 5975 | 1583130 | 1583130 | 1583053 | Seq. ID No. 5976 |
| 2846a | Seq. ID No. 5977 | 1584739 | 1584682 | 1583180 | Seq. ID No. 5978 |
| 2846b | Seq. ID No. 5979 | 1584739 | 1584736 | 1583180 | Seq. ID No. 5980 |
| 2848 | Seq. ID No. 5981 | 1585873 | 1585864 | 1585124 | Seq. ID No. 5982 |
| 2850 | Seq. ID No. 5983 | 1586969 | 1586951 | 1586739 | Seq. ID No. 5984 |
| 2851 | Seq. ID No. 5985 | 1588973 | 1588943 | 1587099 | Seq. ID No. 5986 |
| 2852 | Seq. ID No. 5987 | 1587816 | 1587849 | 1588103 | Seq. ID No. 5988 |
| 2853 | Seq. ID No. 5989 | 1589963 | 1589963 | 1588974 | Seq. ID No. 5990 |
| 2854a | Seq. ID No. 5991 | 1590804 | 1590726 | 1590217 | Seq. ID No. 5992 |
| 2854b | Seq. ID No. 5993 | 1590804 | 1590798 | 1590217 | Seq. ID No. 5994 |
| 2855 | Seq. ID No. 5995 | 1591410 | 1591476 | 1591931 | Seq. ID No. 5996 |
| 2856a | Seq. ID No. 5997 | 1591867 | 1591996 | 1594047 | Seq. ID No. 5998 |
| 2856b | Seq. ID No. 5999 | 1591867 | 1591945 | 1594047 | Seq. ID No. 6000 |
| 2856c | Seq. ID No. 6001 | 1591867 | 1591900 | 1594047 | Seq. ID No. 6002 |
| 2857 | Seq. ID No. 6003 | 1595645 | 1595627 | 1594251 | Seq. ID No. 6004 |
| 2859 | Seq. ID No. 6005 | 1595626 | 1595626 | 1595733 | Seq. ID No. 6006 |
| 2860a | Seq. ID No. 6007 | 1597299 | 1597131 | 1596229 | Seq. ID No. 6008 |
| 2860b | Seq. ID No. 6009 | 1597299 | 1597275 | 1596229 | Seq. ID No. 6010 |
| 2860c | Seq. ID No. 6011 | 1597299 | 1597293 | 1596229 | Seq. ID No. 6012 |
| 2862 | Seq. ID No. 6013 | 1596809 | 1596821 | 1597177 | Seq. ID No. 6014 |
| 2863 | Seq. ID No. 6015 | 1598748 | 1598733 | 1597300 | Seq. ID No. 6016 |
| 2864 | Seq. ID No. 6017 | 1600262 | 1600202 | 1598736 | Seq. ID No. 6018 |
| 2868a | Seq. ID No. 6019 | 1600543 | 1600507 | 1600202 | Seq. ID No. 6020 |
| 2868b | Seq. ID No. 6021 | 1600543 | 1600525 | 1600202 | Seq. ID No. 6022 |
| 2869a | Seq. ID No. 6023 | 1601667 | 1601622 | 1600540 | Seq. ID No. 6024 |
| 2869b | Seq. ID No. 6025 | 1601667 | 1601646 | 1600540 | Seq. ID No. 6026 |
| 2869c | Seq. ID No. 6027 | 1601667 | 1601658 | 1600540 | Seq. ID No. 6028 |
| 2871 | Seq. ID No. 6029 | 1603689 | 1603686 | 1601668 | Seq. ID No. 6030 |
| 2872 | Seq. ID No. 6031 | 1601990 | 1602041 | 1602271 | Seq. ID No. 6032 |
| 2875 | Seq. ID No. 6033 | 1605944 | 1605923 | 1603701 | Seq. ID No. 6034 |
| 2877 | Seq. ID No. 6035 | 1606804 | 1606738 | 1606094 | Seq. ID No. 6036 |
| 2879a | Seq. ID No. 6037 | 1607607 | 1607562 | 1606828 | Seq. ID No. 6038 |
| 2879b | Seq. ID No. 6039 | 1607607 | 1607583 | 1606828 | Seq. ID No. 6040 |
| 2879c | Seq. ID No. 6041 | 1607607 | 1607607 | 1606828 | Seq. ID No. 6042 |
| 2881 | Seq. ID No. 6043 | 1608286 | 1608187 | 1607564 | Seq. ID No. 6044 |
| 2882 | Seq. ID No. 6045 | 1607771 | 1607807 | 1608046 | Seq. ID No. 6046 |
| 2883a | Seq. ID No. 6047 | 1611163 | 1609828 | 1608341 | Seq. ID No. 6048 |
| 2883b | Seq. ID No. 6049 | 1611163 | 1611025 | 1608341 | Seq. ID No. 6050 |
| 2883c | Seq. ID No. 6051 | 1611163 | 1611046 | 1608341 | Seq. ID No. 6052 |
| 2883d | Seq. ID No. 6053 | 1611163 | 1611157 | 1608341 | Seq. ID No. 6054 |
| 2886a | Seq. ID No. 6055 | 1611898 | 1611889 | 1611185 | Seq. ID No. 6056 |
| 2886b | Seq. ID No. 6057 | 1611898 | 1611898 | 1611185 | Seq. ID No. 6058 |
| 2887a | Seq. ID No. 6059 | 1611006 | 1611216 | 1611500 | Seq. ID No. 6060 |
| 2887b | Seq. ID No. 6061 | 1611006 | 1611024 | 1611500 | Seq. ID No. 6062 |
| 2888 | Seq. ID No. 6063 | 1611669 | 1611672 | 1611914 | Seq. ID No. 6064 |
| 2890 | Seq. ID No. 6065 | 1612042 | 1612057 | 1612656 | Seq. ID No. 6066 |
| 2891a | Seq. ID No. 6067 | 1613956 | 1613311 | 1612760 | Seq. ID No. 6068 |
| 2891b | Seq. ID No. 6069 | 1613956 | 1613935 | 1612760 | Seq. ID No. 6070 |
| 2891c | Seq. ID No. 6071 | 1613956 | 1613944 | 1612760 | Seq. ID No. 6072 |
| 2893b | Seq. ID No. 6073 | 1613327 | 1613333 | 1613815 | Seq. ID No. 6074 |
| 2894a | Seq. ID No. 6075 | 1614952 | 1614535 | 1613957 | Seq. ID No. 6076 |
| 2894b | Seq. ID No. 6077 | 1614952 | 1614928 | 1613957 | Seq. ID No. 6078 |
| 2894c | Seq. ID No. 6079 | 1614952 | 1614931 | 1613957 | Seq. ID No. 6080 |
| 2895 | Seq. ID No. 6081 | 1614554 | 1614620 | 1614847 | Seq. ID No. 6082 |
| 2897a | Seq. ID No. 6083 | 1616609 | 1616564 | 1615302 | Seq. ID No. 6084 |
| 2897b | Seq. ID No. 6085 | 1616609 | 1616597 | 1615302 | Seq. ID No. 6086 |
| 2900a | Seq. ID No. 6087 | 1618165 | 1618144 | 1617167 | Seq. ID No. 6088 |
| 2900b | Seq. ID No. 6089 | 1618165 | 1618150 | 1617167 | Seq. ID No. 6090 |
| 2901a | Seq. ID No. 6091 | 1617668 | 1617746 | 1618075 | Seq. ID No. 6092 |
| 2901b | Seq. ID No. 6093 | 1617668 | 1617680 | 1618075 | Seq. ID No. 6094 |
| 2902 | Seq. ID No. 6095 | 1619717 | 1619714 | 1618173 | Seq. ID No. 6096 |
| 2904 | Seq. ID No. 6097 | 1620381 | 1620264 | 1619935 | Seq. ID No. 6098 |
| 2905a | Seq. ID No. 6099 | 1621305 | 1620918 | 1620382 | Seq. ID No. 6100 |
| 2905b | Seq. ID No. 6101 | 1621305 | 1621284 | 1620382 | Seq. ID No. 6102 |
| 2906 | Seq. ID No. 6103 | 1623352 | 1623340 | 1621463 | Seq. ID No. 6104 |
| 2909a | Seq. ID No. 6105 | 1625836 | 1625236 | 1623602 | Seq. ID No. 6106 |
| 2909b | Seq. ID No. 6107 | 1625836 | 1625401 | 1623602 | Seq. ID No. 6108 |
| 2909c | Seq. ID No. 6109 | 1625836 | 1625821 | 1623602 | Seq. ID No. 6110 |
| 2909d | Seq. ID No. 6111 | 1625836 | 1625827 | 1623602 | Seq. ID No. 6112 |
| 2912 | Seq. ID No. 6113 | 1626173 | 1626179 | 1626469 | Seq. ID No. 6114 |
| 2913a | Seq. ID No. 6115 | 1628027 | 1627994 | 1626564 | Seq. ID No. 6116 |
| 2913b | Seq. ID No. 6117 | 1628027 | 1628021 | 1626564 | Seq. ID No. 6118 |
| 2915a | Seq. ID No. 6119 | 1628740 | 1628713 | 1628024 | Seq. ID No. 6120 |
| 2915b | Seq. ID No. 6121 | 1628740 | 1628725 | 1628024 | Seq. ID No. 6122 |
| 2915c | Seq. ID No. 6123 | 1628740 | 1628740 | 1628024 | Seq. ID No. 6124 |
| 2916 | Seq. ID No. 6125 | 1629233 | 1629218 | 1628859 | Seq. ID No. 6126 |
| 2917 | Seq. ID No. 6127 | 1629566 | 1629566 | 1629375 | Seq. ID No. 6128 |
| 2918 | Seq. ID No. 6129 | 1631092 | 1631089 | 1629779 | Seq. ID No. 6130 |
| 2920 | Seq. ID No. 6131 | 1631506 | 1631473 | 1631243 | Seq. ID No. 6132 |
| 2921 | Seq. ID No. 6133 | 1632233 | 1632218 | 1631589 | Seq. ID No. 6134 |
| 2922b | Seq. ID No. 6135 | 1631644 | 1631647 | 1631913 | Seq. ID No. 6136 |
| 2923a | Seq. ID No. 6137 | 1633273 | 1632916 | 1632269 | Seq. ID No. 6138 |
| 2923b | Seq. ID No. 6139 | 1633273 | 1633258 | 1632269 | Seq. ID No. 6140 |
| 2923c | Seq. ID No. 6141 | 1633273 | 1633267 | 1632269 | Seq. ID No. 6142 |
| 2925a | Seq. ID No. 6143 | 1634281 | 1634122 | 1633424 | Seq. ID No. 6144 |
| 2925b | Seq. ID No. 6145 | 1634281 | 1634278 | 1633424 | Seq. ID No. 6146 |
| 2926a | Seq. ID No. 6147 | 1635443 | 1635365 | 1634265 | Seq. ID No. 6148 |
| 2926b | Seq. ID No. 6149 | 1635443 | 1635383 | 1634265 | Seq. ID No. 6150 |
| 2926c | Seq. ID No. 6151 | 1635443 | 1635437 | 1634265 | Seq. ID No. 6152 |
| 2928a | Seq. ID No. 6153 | 1635990 | 1635924 | 1635358 | Seq. ID No. 6154 |
| 2928b | Seq. ID No. 6155 | 1635990 | 1635954 | 1635358 | Seq. ID No. 6156 |
| 2928c | Seq. ID No. 6157 | 1635990 | 1635963 | 1635358 | Seq. ID No. 6158 |
| 2929b | Seq. ID No. 6159 | 1635214 | 1635241 | 1635663 | Seq. ID No. 6160 |
| 2931a | Seq. ID No. 6161 | 1637677 | 1636687 | 1636097 | Seq. ID No. 6162 |
| 2931b | Seq. ID No. 6163 | 1637677 | 1636831 | 1636097 | Seq. ID No. 6164 |
| 2931c | Seq. ID No. 6165 | 1637677 | 1637605 | 1636097 | Seq. ID No. 6166 |
| 2933a | Seq. ID No. 6167 | 1639308 | 1639119 | 1637683 | Seq. ID No. 6168 |
| 2933b | Seq. ID No. 6169 | 1639308 | 1639299 | 1637683 | Seq. ID No. 6170 |
| 2934 | Seq. ID No. 6171 | 1640421 | 1640421 | 1639309 | Seq. ID No. 6172 |
| 2935a | Seq. ID No. 6173 | 1641368 | 1641266 | 1640808 | Seq. ID No. 6174 |
| 2935b | Seq. ID No. 6175 | 1641368 | 1641365 | 1640808 | Seq. ID No. 6176 |
| 2936 | Seq. ID No. 6177 | 1642293 | 1642278 | 1641352 | Seq. ID No. 6178 |
| 2939a | Seq. ID No. 6179 | 1642920 | 1642842 | 1642435 | Seq. ID No. 6180 |
| 2939b | Seq. ID No. 6181 | 1642920 | 1642902 | 1642435 | Seq. ID No. 6182 |
| 2941a | Seq. ID No. 6183 | 1645387 | 1644805 | 1643459 | Seq. ID No. 6184 |
| 2941b | Seq. ID No. 6185 | 1645387 | 1645246 | 1643459 | Seq. ID No. 6186 |
| 2941c | Seq. ID No. 6187 | 1645387 | 1645318 | 1643459 | Seq. ID No. 6188 |
| 2943a | Seq. ID No. 6189 | 1647063 | 1646619 | 1645318 | Seq. ID No. 6190 |
| 2943b | Seq. ID No. 6191 | 1647063 | 1646910 | 1645318 | Seq. ID No. 6192 |
| 2943c | Seq. ID No. 6193 | 1647063 | 1647036 | 1645318 | Seq. ID No. 6194 |
| 2943d | Seq. ID No. 6195 | 1647063 | 1647042 | 1645318 | Seq. ID No. 6196 |
| 2944b | Seq. ID No. 6197 | 1645364 | 1645379 | 1645759 | Seq. ID No. 6198 |
| 2945b | Seq. ID No. 6199 | 1646411 | 1646414 | 1646893 | Seq. ID No. 6200 |
| 2947 | Seq. ID No. 6201 | 1647671 | 1647659 | 1647036 | Seq. ID No. 6202 |
| 2948a | Seq. ID No. 6203 | 1648994 | 1648082 | 1647831 | Seq. ID No. 6204 |
| 2948b | Seq. ID No. 6205 | 1648994 | 1648781 | 1647831 | Seq. ID No. 6206 |
| 2948c | Seq. ID No. 6207 | 1648994 | 1648886 | 1647831 | Seq. ID No. 6208 |
| 2948d | Seq. ID No. 6209 | 1648994 | 1648985 | 1647831 | Seq. ID No. 6210 |
| 2950a | Seq. ID No. 6211 | 1649095 | 1649113 | 1650147 | Seq. ID No. 6212 |
| 2950b | Seq. ID No. 6213 | 1649095 | 1649095 | 1650147 | Seq. ID No. 6214 |
| 2951 | Seq. ID No. 6215 | 1650757 | 1650751 | 1650221 | Seq. ID No. 6216 |
| 2952 | Seq. ID No. 6217 | 1650135 | 1650153 | 1650560 | Seq. ID No. 6218 |
| 2954 | Seq. ID No. 6219 | 1650935 | 1650944 | 1651768 | Seq. ID No. 6220 |
| 2955a | Seq. ID No. 6221 | 1653168 | 1652175 | 1651840 | Seq. ID No. 6222 |
| 2955b | Seq. ID No. 6223 | 1653168 | 1652187 | 1651840 | Seq. ID No. 6224 |
| 2955c | Seq. ID No. 6225 | 1653168 | 1652409 | 1651840 | Seq. ID No. 6226 |
| 2955d | Seq. ID No. 6227 | 1653168 | 1652565 | 1651840 | Seq. ID No. 6228 |
| 2955e | Seq. ID No. 6229 | 1653168 | 1652973 | 1651840 | Seq. ID No. 6230 |
| 2955f | Seq. ID No. 6231 | 1653168 | 1653084 | 1651840 | Seq. ID No. 6232 |
| 2955g | Seq. ID No. 6233 | 1653168 | 1653168 | 1651840 | Seq. ID No. 6234 |
| 2957 | Seq. ID No. 6235 | 1653355 | 1653376 | 1653993 | Seq. ID No. 6236 |
| 2958a | Seq. ID No. 6237 | 1654895 | 1654766 | 1654368 | Seq. ID No. 6238 |
| 2958b | Seq. ID No. 6239 | 1654895 | 1654856 | 1654368 | Seq. ID No. 6240 |
| 2958c | Seq. ID No. 6241 | 1654895 | 1654880 | 1654368 | Seq. ID No. 6242 |
| 2959 | Seq. ID No. 6243 | 1653994 | 1653997 | 1654986 | Seq. ID No. 6244 |
| 2960a | Seq. ID No. 6245 | 1655465 | 1655390 | 1655130 | Seq. ID No. 6246 |
| 2962a | Seq. ID No. 6247 | 1654995 | 1655103 | 1655627 | Seq. ID No. 6248 |
| 2962b | Seq. ID No. 6249 | 1654995 | 1654995 | 1655627 | Seq. ID No. 6250 |
| 2963 | Seq. ID No. 6251 | 1655942 | 1655954 | 1656421 | Seq. ID No. 6252 |
| 2965a | Seq. ID No. 6253 | 1658340 | 1657503 | 1657087 | Seq. ID No. 6254 |
| 2965b | Seq. ID No. 6255 | 1658340 | 1658307 | 1657087 | Seq. ID No. 6256 |
| 2965c | Seq. ID No. 6257 | 1658340 | 1658325 | 1657087 | Seq. ID No. 6258 |
| 2966 | Seq. ID No. 6259 | 1658084 | 1658096 | 1658344 | Seq. ID No. 6260 |
| 2967 | Seq. ID No. 6261 | 1660896 | 1660872 | 1658707 | Seq. ID No. 6262 |
| 2969 | Seq. ID No. 6263 | 1661016 | 1661061 | 1661981 | Seq. ID No. 6264 |
| 2970a | Seq. ID No. 6265 | 1663149 | 1662966 | 1662703 | Seq. ID No. 6266 |
| 2970b | Seq. ID No. 6267 | 1663149 | 1662999 | 1662703 | Seq. ID No. 6268 |
| 2971a | Seq. ID No. 6269 | 1661991 | 1662033 | 1662746 | Seq. ID No. 6270 |
| 2971b | Seq. ID No. 6271 | 1661991 | 1661991 | 1662746 | Seq. ID No. 6272 |
| 2974a | Seq. ID No. 6273 | 1662698 | 1663724 | 1663963 | Seq. ID No. 6274 |
| 2974b | Seq. ID No. 6275 | 1662698 | 1662743 | 1663963 | Seq. ID No. 6276 |
| 2975 | Seq. ID No. 6277 | 1665419 | 1665395 | 1664037 | Seq. ID No. 6278 |
| 2977a | Seq. ID No. 6279 | 1665491 | 1665566 | 1666129 | Seq. ID No. 6280 |
| 2977b | Seq. ID No. 6281 | 1665491 | 1665506 | 1666129 | Seq. ID No. 6282 |
| 2978a | Seq. ID No. 6283 | 1666409 | 1666355 | 1666131 | Seq. ID No. 6284 |
| 2978b | Seq. ID No. 6285 | 1666409 | 1666364 | 1666131 | Seq. ID No. 6286 |
| 2979 | Seq. ID No. 6287 | 1666051 | 1666126 | 1666689 | Seq. ID No. 6288 |
| 2980a | Seq. ID No. 6289 | 1668091 | 1667302 | 1666901 | Seq. ID No. 6290 |
| 2980b | Seq. ID No. 6291 | 1668091 | 1667875 | 1666901 | Seq. ID No. 6292 |
| 2980c | Seq. ID No. 6293 | 1668091 | 1667995 | 1666901 | Seq. ID No. 6294 |
| 2980d | Seq. ID No. 6295 | 1668091 | 1668028 | 1666901 | Seq. ID No. 6296 |
| 2980e | Seq. ID No. 6297 | 1668091 | 1668070 | 1666901 | Seq. ID No. 6298 |
| 2984 | Seq. ID No. 6299 | 1669334 | 1669304 | 1667970 | Seq. ID No. 6300 |
| 2986a | Seq. ID No. 6301 | 1670826 | 1670514 | 1669684 | Seq. ID No. 6302 |
| 2986b | Seq. ID No. 6303 | 1670826 | 1670706 | 1669684 | Seq. ID No. 6304 |
| 2986c | Seq. ID No. 6305 | 1670826 | 1670790 | 1669684 | Seq. ID No. 6306 |
| 2988a | Seq. ID No. 6307 | 1672193 | 1672121 | 1670793 | Seq. ID No. 6308 |
| 2988b | Seq. ID No. 6309 | 1672193 | 1672148 | 1670793 | Seq. ID No. 6310 |
| 2988c | Seq. ID No. 6311 | 1672193 | 1672154 | 1670793 | Seq. ID No. 6312 |
| 2989 | Seq. ID No. 6313 | 1672635 | 1672536 | 1672342 | Seq. ID No. 6314 |
| 2990 | Seq. ID No. 6315 | 1674173 | 1674143 | 1672551 | Seq. ID No. 6316 |
| 2991a | Seq. ID No. 6317 | 1675155 | 1674828 | 1674154 | Seq. ID No. 6318 |
| 2991b | Seq. ID No. 6319 | 1675155 | 1675080 | 1674154 | Seq. ID No. 6320 |
| 2991c | Seq. ID No. 6321 | 1675155 | 1675140 | 1674154 | Seq. ID No. 6322 |
| 2992 | Seq. ID No. 6323 | 1675279 | 1675294 | 1675782 | Seq. ID No. 6324 |
| 2993a | Seq. ID No. 6325 | 1676636 | 1676624 | 1675920 | Seq. ID No. 6326 |
| 2993b | Seq. ID No. 6327 | 1676636 | 1676627 | 1675920 | Seq. ID No. 6328 |
| 2995a | Seq. ID No. 6329 | 1677557 | 1677437 | 1676643 | Seq. ID No. 6330 |
| 2995b | Seq. ID No. 6331 | 1677557 | 1677554 | 1676643 | Seq. ID No. 6332 |
| 2997 | Seq. ID No. 6333 | 1677875 | 1677869 | 1677558 | Seq. ID No. 6334 |
| 2998a | Seq. ID No. 6335 | 1678755 | 1678617 | 1677862 | Seq. ID No. 6336 |
| 2998b | Seq. ID No. 6337 | 1678755 | 1678701 | 1677862 | Seq. ID No. 6338 |
| 2999 | Seq. ID No. 6339 | 1679123 | 1679120 | 1678734 | Seq. ID No. 6340 |
| 3001 | Seq. ID No. 6341 | 1680254 | 1680221 | 1679229 | Seq. ID No. 6342 |
| 3003 | Seq. ID No. 6343 | 1680586 | 1680574 | 1680245 | Seq. ID No. 6344 |
| 3005 | Seq. ID No. 6345 | 1681275 | 1681266 | 1680622 | Seq. ID No. 6346 |
| 3008a | Seq. ID No. 6347 | 1681972 | 1681849 | 1681283 | Seq. ID No. 6348 |
| 3008b | Seq. ID No. 6349 | 1681972 | 1681879 | 1681283 | Seq. ID No. 6350 |
| 3008c | Seq. ID No. 6351 | 1681972 | 1681882 | 1681283 | Seq. ID No. 6352 |
| 3008d | Seq. ID No. 6353 | 1681972 | 1681936 | 1681283 | Seq. ID No. 6354 |
| 3010a | Seq. ID No. 6355 | 1682354 | 1682306 | 1681995 | Seq. ID No. 6356 |
| 3010b | Seq. ID No. 6357 | 1682354 | 1682315 | 1681995 | Seq. ID No. 6358 |
| 3012 | Seq. ID No. 6359 | 1684114 | 1684102 | 1682330 | Seq. ID No. 6360 |
| 3015 | Seq. ID No. 6361 | 1684647 | 1684692 | 1685450 | Seq. ID No. 6362 |
| 3016a | Seq. ID No. 6363 | 1686951 | 1686111 | 1685629 | Seq. ID No. 6364 |
| 3016b | Seq. ID No. 6365 | 1686951 | 1686861 | 1685629 | Seq. ID No. 6366 |
| 3016c | Seq. ID No. 6367 | 1686951 | 1686930 | 1685629 | Seq. ID No. 6368 |
| 3017 | Seq. ID No. 6369 | 1687931 | 1687823 | 1686927 | Seq. ID No. 6370 |
| 3018a | Seq. ID No. 6371 | 1688418 | 1688376 | 1687897 | Seq. ID No. 6372 |
| 3018b | Seq. ID No. 6373 | 1688418 | 1688379 | 1687897 | Seq. ID No. 6374 |
| 3020 | Seq. ID No. 6375 | 1689300 | 1689261 | 1688419 | Seq. ID No. 6376 |
| 3021 | Seq. ID No. 6377 | 1689758 | 1689743 | 1689423 | Seq. ID No. 6378 |
| 3022 | Seq. ID No. 6379 | 1692324 | 1692324 | 1689814 | Seq. ID No. 6380 |
| 3023 | Seq. ID No. 6381 | 1689787 | 1689787 | 1690059 | Seq. ID No. 6382 |
| 3024a | Seq. ID No. 6383 | 1691465 | 1691537 | 1691806 | Seq. ID No. 6384 |
| 3024b | Seq. ID No. 6385 | 1691465 | 1691474 | 1691806 | Seq. ID No. 6386 |
| 3026a | Seq. ID No. 6387 | 1692828 | 1692804 | 1692346 | Seq. ID No. 6388 |
| 3026b | Seq. ID No. 6389 | 1692828 | 1692819 | 1692346 | Seq. ID No. 6390 |
| 3028a | Seq. ID No. 6391 | 1693823 | 1693703 | 1693047 | Seq. ID No. 6392 |
| 3028b | Seq. ID No. 6393 | 1693823 | 1693709 | 1693047 | Seq. ID No. 6394 |
| 3028c | Seq. ID No. 6395 | 1693823 | 1693763 | 1693047 | Seq. ID No. 6396 |
| 3029a | Seq. ID No. 6397 | 1694761 | 1694746 | 1693703 | Seq. ID No. 6398 |
| 3029b | Seq. ID No. 6399 | 1694761 | 1694758 | 1693703 | Seq. ID No. 6400 |
| 3031a | Seq. ID No. 6401 | 1695379 | 1695412 | 1695777 | Seq. ID No. 6402 |
| 3031b | Seq. ID No. 6403 | 1695379 | 1695394 | 1695777 | Seq. ID No. 6404 |
| 3032a | Seq. ID No. 6405 | 1696446 | 1696257 | 1696033 | Seq. ID No. 6406 |
| 3032b | Seq. ID No. 6407 | 1696446 | 1696299 | 1696033 | Seq. ID No. 6408 |
| 3033a | Seq. ID No. 6409 | 1697227 | 1697194 | 1696382 | Seq. ID No. 6410 |
| 3033b | Seq. ID No. 6411 | 1697227 | 1697197 | 1696382 | Seq. ID No. 6412 |
| 3035 | Seq. ID No. 6413 | 1697987 | 1697987 | 1697232 | Seq. ID No. 6414 |
| 3037a | Seq. ID No. 6415 | 1698709 | 1698781 | 1699263 | Seq. ID No. 6416 |
| 3037b | Seq. ID No. 6417 | 1698709 | 1698739 | 1699263 | Seq. ID No. 6418 |
| 3038 | Seq. ID No. 6419 | 1699841 | 1699841 | 1700038 | Seq. ID No. 6420 |
| 3040a | Seq. ID No. 6421 | 1701079 | 1700950 | 1700138 | Seq. ID No. 6422 |
| 3040b | Seq. ID No. 6423 | 1701079 | 1701079 | 1700138 | Seq. ID No. 6424 |
| 3042a | Seq. ID No. 6425 | 1700433 | 1700445 | 1700675 | Seq. ID No. 6426 |
| 3042b | Seq. ID No. 6427 | 1700433 | 1700433 | 1700675 | Seq. ID No. 6428 |
| 3043a | Seq. ID No. 6429 | 1701375 | 1701306 | 1701073 | Seq. ID No. 6430 |
| 3043b | Seq. ID No. 6431 | 1701375 | 1701324 | 1701073 | Seq. ID No. 6432 |
| 3044 | Seq. ID No. 6433 | 1701638 | 1701617 | 1701321 | Seq. ID No. 6434 |
| 3045a | Seq. ID No. 6435 | 1702055 | 1702040 | 1701639 | Seq. ID No. 6436 |
| 3045b | Seq. ID No. 6437 | 1702055 | 1702043 | 1701639 | Seq. ID No. 6438 |
| 3048a | Seq. ID No. 6439 | 1702494 | 1702383 | 1702033 | Seq. ID No. 6440 |
| 3049a | Seq. ID No. 6441 | 1704304 | 1703878 | 1702388 | Seq. ID No. 6442 |
| 3049b | Seq. ID No. 6443 | 1704304 | 1704055 | 1702388 | Seq. ID No. 6444 |
| 3049c | Seq. ID No. 6445 | 1704304 | 1704289 | 1702388 | Seq. ID No. 6446 |
| 3051a | Seq. ID No. 6447 | 1704655 | 1704649 | 1704305 | Seq. ID No. 6448 |
| 3051b | Seq. ID No. 6449 | 1704655 | 1704655 | 1704305 | Seq. ID No. 6450 |
| 3052 | Seq. ID No. 6451 | 1706095 | 1706095 | 1704656 | Seq. ID No. 6452 |
| 3054 | Seq. ID No. 6453 | 1705443 | 1705446 | 1705685 | Seq. ID No. 6454 |
| 3055a | Seq. ID No. 6455 | 1706386 | 1706362 | 1706096 | Seq. ID No. 6456 |
| 3055b | Seq. ID No. 6457 | 1706386 | 1706383 | 1706096 | Seq. ID No. 6458 |
| 3057 | Seq. ID No. 6459 | 1708245 | 1708242 | 1706380 | Seq. ID No. 6460 |
| 3059 | Seq. ID No. 6461 | 1709019 | 1709007 | 1708246 | Seq. ID No. 6462 |
| 3060 | Seq. ID No. 6463 | 1711662 | 1711659 | 1709020 | Seq. ID No. 6464 |
| 3061 | Seq. ID No. 6465 | 1709528 | 1709528 | 1709259 | Seq. ID No. 6466 |
| 3062 | Seq. ID No. 6467 | 1709324 | 1709330 | 1710160 | Seq. ID No. 6468 |
| 3063a | Seq. ID No. 6469 | 1710350 | 1710368 | 1710655 | Seq. ID No. 6470 |
| 3063b | Seq. ID No. 6471 | 1710350 | 1710365 | 1710655 | Seq. ID No. 6472 |
| 3064b | Seq. ID No. 6473 | 1710920 | 1710938 | 1711330 | Seq. ID No. 6474 |
| 3065a | Seq. ID No. 6475 | 1712082 | 1711980 | 1711663 | Seq. ID No. 6476 |
| 3065b | Seq. ID No. 6477 | 1712082 | 1711983 | 1711663 | Seq. ID No. 6478 |
| 3066 | Seq. ID No. 6479 | 1711331 | 1711412 | 1711774 | Seq. ID No. 6480 |
| 3067 | Seq. ID No. 6481 | 1712393 | 1712384 | 1712001 | Seq. ID No. 6482 |
| 3068 | Seq. ID No. 6483 | 1713000 | 1712988 | 1712401 | Seq. ID No. 6484 |
| 3069 | Seq. ID No. 6485 | 1713434 | 1713380 | 1712991 | Seq. ID No. 6486 |
| 3071 | Seq. ID No. 6487 | 1714045 | 1714003 | 1713764 | Seq. ID No. 6488 |
| 3072a | Seq. ID No. 6489 | 1714353 | 1714293 | 1714000 | Seq. ID No. 6490 |
| 3073 | Seq. ID No. 6491 | 1714645 | 1714639 | 1714304 | Seq. ID No. 6492 |
| 3074 | Seq. ID No. 6493 | 1715783 | 1715780 | 1714785 | Seq. ID No. 6494 |
| 3075 | Seq. ID No. 6495 | 1716350 | 1716335 | 1715784 | Seq. ID No. 6496 |
| 3076 | Seq. ID No. 6497 | 1715964 | 1716024 | 1716341 | Seq. ID No. 6498 |
| 3077a | Seq. ID No. 6499 | 1717443 | 1717344 | 1716424 | Seq. ID No. 6500 |
| 3077b | Seq. ID No. 6501 | 1717443 | 1717425 | 1716424 | Seq. ID No. 6502 |
| 3077c | Seq. ID No. 6503 | 1717443 | 1717443 | 1716424 | Seq. ID No. 6504 |
| 3078 | Seq. ID No. 6505 | 1718902 | 1718866 | 1717376 | Seq. ID No. 6506 |
| 3079 | Seq. ID No. 6507 | 1717769 | 1717769 | 1718020 | Seq. ID No. 6508 |
| 3081a | Seq. ID No. 6509 | 1720200 | 1720161 | 1718881 | Seq. ID No. 6510 |
| 3081b | Seq. ID No. 6511 | 1720200 | 1720179 | 1718881 | Seq. ID No. 6512 |
| 3083c | Seq. ID No. 6513 | 1720609 | 1720570 | 1720154 | Seq. ID No. 6514 |
| 3083d | Seq. ID No. 6515 | 1720609 | 1720588 | 1720154 | Seq. ID No. 6516 |
| 3085 | Seq. ID No. 6517 | 1720239 | 1720287 | 1720517 | Seq. ID No. 6518 |
| 3086 | Seq. ID No. 6519 | 1721223 | 1721223 | 1720798 | Seq. ID No. 6520 |
| 3087a | Seq. ID No. 6521 | 1721741 | 1721516 | 1721220 | Seq. ID No. 6522 |
| 3087c | Seq. ID No. 6523 | 1721741 | 1721729 | 1721220 | Seq. ID No. 6524 |
| 3088 | Seq. ID No. 6525 | 1722323 | 1722311 | 1721742 | Seq. ID No. 6526 |
| 3089a | Seq. ID No. 6527 | 1722579 | 1722534 | 1722301 | Seq. ID No. 6528 |
| 3089b | Seq. ID No. 6529 | 1722579 | 1722540 | 1722301 | Seq. ID No. 6530 |
| 3090 | Seq. ID No. 6531 | 1723087 | 1723024 | 1722710 | Seq. ID No. 6532 |
| 3091 | Seq. ID No. 6533 | 1723434 | 1723377 | 1723024 | Seq. ID No. 6534 |
| 3092a | Seq. ID No. 6535 | 1726056 | 1726017 | 1723726 | Seq. ID No. 6536 |
| 3092b | Seq. ID No. 6537 | 1726056 | 1726023 | 1723726 | Seq. ID No. 6538 |
| 3095 | Seq. ID No. 6539 | 1725374 | 1725374 | 1724937 | Seq. ID No. 6540 |
| 3096a | Seq. ID No. 6541 | 1724735 | 1724747 | 1724995 | Seq. ID No. 6542 |
| 3096b | Seq. ID No. 6543 | 1724735 | 1724738 | 1724995 | Seq. ID No. 6544 |
| 3097 | Seq. ID No. 6545 | 1726568 | 1726568 | 1726035 | Seq. ID No. 6546 |
| 3098a | Seq. ID No. 6547 | 1728002 | 1727765 | 1726569 | Seq. ID No. 6548 |
| 3098b | Seq. ID No. 6549 | 1728002 | 1727993 | 1726569 | Seq. ID No. 6550 |
| 3098c | Seq. ID No. 6551 | 1728002 | 1728002 | 1726569 | Seq. ID No. 6552 |
| 3099 | Seq. ID No. 6553 | 1728681 | 1728669 | 1727986 | Seq. ID No. 6554 |
| 3100a | Seq. ID No. 6555 | 1727960 | 1728044 | 1728307 | Seq. ID No. 6556 |
| 3100b | Seq. ID No. 6557 | 1727960 | 1728017 | 1728307 | Seq. ID No. 6558 |
| 3101 | Seq. ID No. 6559 | 1729161 | 1729158 | 1728682 | Seq. ID No. 6560 |
| 3102 | Seq. ID No. 6561 | 1729471 | 1729429 | 1729220 | Seq. ID No. 6562 |
| 3103b | Seq. ID No. 6563 | 1729917 | 1729917 | 1729426 | Seq. ID No. 6564 |
| 3104 | Seq. ID No. 6565 | 1730101 | 1730101 | 1729931 | Seq. ID No. 6566 |
| 3105 | Seq. ID No. 6567 | 1730458 | 1730434 | 1730144 | Seq. ID No. 6568 |
| 3106 | Seq. ID No. 6569 | 1730695 | 1730668 | 1730465 | Seq. ID No. 6570 |
| 3107a | Seq. ID No. 6571 | 1731400 | 1731364 | 1731125 | Seq. ID No. 6572 |
| 3107b | Seq. ID No. 6573 | 1731400 | 1731385 | 1731125 | Seq. ID No. 6574 |
| 3108a | Seq. ID No. 6575 | 1730751 | 1730838 | 1731467 | Seq. ID No. 6576 |
| 3108b | Seq. ID No. 6577 | 1730751 | 1730778 | 1731467 | Seq. ID No. 6578 |
| 3109 | Seq. ID No. 6579 | 1731554 | 1731557 | 1732228 | Seq. ID No. 6580 |
| 3110a | Seq. ID No. 6581 | 1732232 | 1732322 | 1732933 | Seq. ID No. 6582 |
| 3110b | Seq. ID No. 6583 | 1732232 | 1732244 | 1732933 | Seq. ID No. 6584 |
| 3113 | Seq. ID No. 6585 | 1733078 | 1733090 | 1734292 | Seq. ID No. 6586 |
| 3116 | Seq. ID No. 6587 | 1734912 | 1734921 | 1735769 | Seq. ID No. 6588 |
| 3118a | Seq. ID No. 6589 | 1735601 | 1735763 | 1736248 | Seq. ID No. 6590 |
| 3118b | Seq. ID No. 6591 | 1735601 | 1735673 | 1736248 | Seq. ID No. 6592 |
| 3119a | Seq. ID No. 6593 | 1736902 | 1736743 | 1736339 | Seq. ID No. 6594 |
| 3119b | Seq. ID No. 6595 | 1736902 | 1736782 | 1736339 | Seq. ID No. 6596 |
| 3119c | Seq. ID No. 6597 | 1736902 | 1736809 | 1736339 | Seq. ID No. 6598 |
| 3119d | Seq. ID No. 6599 | 1736902 | 1736848 | 1736339 | Seq. ID No. 6600 |
| 3119e | Seq. ID No. 6601 | 1736902 | 1736863 | 1736339 | Seq. ID No. 6602 |
| 3119f | Seq. ID No. 6603 | 1736902 | 1736872 | 1736339 | Seq. ID No. 6604 |
| 3121 | Seq. ID No. 6605 | 1736957 | 1736978 | 1737658 | Seq. ID No. 6606 |
| 3122a | Seq. ID No. 6607 | 1739218 | 1739212 | 1738022 | Seq. ID No. 6608 |
| 3122b | Seq. ID No. 6609 | 1739218 | 1739218 | 1738022 | Seq. ID No. 6610 |
| 3123a | Seq. ID No. 6611 | 1741016 | 1740947 | 1739265 | Seq. ID No. 6612 |
| 3123b | Seq. ID No. 6613 | 1741016 | 1741007 | 1739265 | Seq. ID No. 6614 |
| 3124 | Seq. ID No. 6615 | 1741584 | 1741551 | 1740970 | Seq. ID No. 6616 |
| 3126 | Seq. ID No. 6617 | 1742270 | 1742243 | 1741596 | Seq. ID No. 6618 |
| 3129a | Seq. ID No. 6619 | 1742959 | 1742902 | 1742246 | Seq. ID No. 6620 |
| 3129b | Seq. ID No. 6621 | 1742959 | 1742932 | 1742246 | Seq. ID No. 6622 |
| 3131 | Seq. ID No. 6623 | 1745173 | 1745167 | 1743977 | Seq. ID No. 6624 |
| 3134a | Seq. ID No. 6625 | 1747570 | 1747468 | 1745222 | Seq. ID No. 6626 |
| 3134b | Seq. ID No. 6627 | 1747570 | 1747507 | 1745222 | Seq. ID No. 6628 |
| 3138 | Seq. ID No. 6629 | 1748892 | 1748832 | 1747537 | Seq. ID No. 6630 |
| 3140 | Seq. ID No. 6631 | 1748324 | 1748327 | 1748560 | Seq. ID No. 6632 |
| 3141 | Seq. ID No. 6633 | 1750186 | 1750177 | 1748972 | Seq. ID No. 6634 |
| 3145a | Seq. ID No. 6635 | 1752277 | 1752205 | 1750973 | Seq. ID No. 6636 |
| 3145b | Seq. ID No. 6637 | 1752277 | 1752259 | 1750973 | Seq. ID No. 6638 |
| 3145c | Seq. ID No. 6639 | 1752277 | 1752274 | 1750973 | Seq. ID No. 6640 |
| 3147a | Seq. ID No. 6641 | 125 | 1754054 | 1752510 | Seq. ID No. 6642 |
| 3147b | Seq. ID No. 6643 | 125 | 122 | 1752510 | Seq. ID No. 6644 |
| 3147c | Seq. ID No. 6645 | 125 | 83 | 1752510 | Seq. ID No. 6646 |
| 3149a | Seq. ID No. 6647 | 7 | 1754326 | 1754123 | Seq. ID No. 6648 |
| 3150a | Seq. ID No. 6649 | 1753936 | 1754086 | 1754331 | Seq. ID No. 6650 |
| 3099 | Seq. ID No. 6553 | 1728681 | 1728669 | 1727986 | Seq. ID No. 6554 |
| 3100a | Seq. ID No. 6555 | 1727960 | 1728044 | 1728307 | Seq. ID No. 6556 |
| 3100b | Seq. ID No. 6557 | 1727960 | 1728017 | 1728307 | Seq. ID No. 6558 |
| 3101 | Seq. ID No. 6559 | 1729161 | 1729158 | 1728682 | Seq. ID No. 6560 |
| 3102 | Seq. ID No. 6561 | 1729471 | 1729429 | 1729220 | Seq. ID No. 6562 |
| 3103b | Seq. ID No. 6563 | 1729917 | 1729917 | 1729426 | Seq. ID No. 6564 |
| 3104 | Seq. ID No. 6565 | 1730101 | 1730101 | 1729931 | Seq. ID No. 6566 |
| 3105 | Seq. ID No. 6567 | 1730458 | 1730434 | 1730144 | Seq. ID No. 6568 |
| 3106 | Seq. ID No. 6569 | 1730695 | 1730668 | 1730465 | Seq. ID No. 6570 |
| 3107a | Seq. ID No. 6571 | 1731400 | 1731364 | 1731125 | Seq. ID No. 6572 |
| 3107b | Seq. ID No. 6573 | 1731400 | 1731385 | 1731125 | Seq. ID No. 6574 |
| 3108a | Seq. ID No. 6575 | 1730751 | 1730838 | 1731467 | Seq. ID No. 6576 |
| 3108b | Seq. ID No. 6577 | 1730751 | 1730778 | 1731467 | Seq. ID No. 6578 |
| 3109 | Seq. ID No. 6579 | 1731554 | 1731557 | 1732228 | Seq. ID No. 6580 |
| 3110a | Seq. ID No. 6581 | 1732232 | 1732322 | 1732933 | Seq. ID No. 6582 |
| 3110b | Seq. ID No. 6583 | 1732232 | 1732244 | 1732933 | Seq. ID No. 6584 |
| 3113 | Seq. ID No. 6585 | 1733078 | 1733090 | 1734292 | Seq. ID No. 6586 |
| 3116 | Seq. ID No. 6587 | 1734912 | 1734921 | 1735769 | Seq. ID No. 6588 |
| 3118a | Seq. ID No. 6589 | 1735601 | 1735763 | 1736248 | Seq. ID No. 6590 |
| 3118b | Seq. ID No. 6591 | 1735601 | 1735673 | 1736248 | Seq. ID No. 6592 |
| 3119a | Seq. ID No. 6593 | 1736902 | 1736743 | 1736339 | Seq. ID No. 6594 |
| 3119b | Seq. ID No. 6595 | 1736902 | 1736782 | 1736339 | Seq. ID No. 6596 |
| 3119c | Seq. ID No. 6597 | 1736902 | 1736809 | 1736339 | Seq. ID No. 6598 |
| 3119d | Seq. ID No. 6599 | 1736902 | 1736848 | 1736339 | Seq. ID No. 6600 |
| 3119e | Seq. ID No. 6601 | 1736902 | 1736863 | 1736339 | Seq. ID No. 6602 |
| 3119f | Seq. ID No. 6603 | 1736902 | 1736872 | 1736339 | Seq. ID No. 6604 |
| 3121 | Seq. ID No. 6605 | 1736957 | 1736978 | 1737658 | Seq. ID No. 6606 |
| 3122a | Seq. ID No. 6607 | 1739218 | 1739212 | 1738022 | Seq. ID No. 6608 |
| 3122b | Seq. ID No. 6609 | 1739218 | 1739218 | 1738022 | Seq. ID No. 6610 |
| 3123a | Seq. ID No. 6611 | 1741016 | 1740947 | 1739265 | Seq. ID No. 6612 |
| 3123b | Seq. ID No. 6613 | 1741016 | 1741007 | 1739265 | Seq. ID No. 6614 |
| 3124 | Seq. ID No. 6615 | 1741584 | 1741551 | 1740970 | Seq. ID No. 6616 |
| 3126 | Seq. ID No. 6617 | 1742270 | 1742243 | 1741596 | Seq. ID No. 6618 |
| 3129a | Seq. ID No. 6619 | 1742959 | 1742902 | 1742246 | Seq. ID No. 6620 |
| 3129b | Seq. ID No. 6621 | 1742959 | 1742932 | 1742246 | Seq. ID No. 6622 |
| 3131 | Seq. ID No. 6623 | 1745173 | 1745167 | 1743977 | Seq. ID No. 6624 |
| 3134a | Seq. ID No. 6625 | 1747570 | 1747468 | 1745222 | Seq. ID No. 6626 |
| 3134b | Seq. ID No. 6627 | 1747570 | 1747507 | 1745222 | Seq. ID No. 6628 |
| 3138 | Seq. ID No. 6629 | 1748892 | 1748832 | 1747537 | Seq. ID No. 6630 |
| 3140 | Seq. ID No. 6631 | 1748324 | 1748327 | 1748560 | Seq. ID No. 6632 |
| 3141 | Seq. ID No. 6633 | 1750186 | 1750177 | 1748972 | Seq. ID No. 6634 |
| 3145a | Seq. ID No. 6635 | 1752277 | 1752205 | 1750973 | Seq. ID No. 6636 |
| 3145b | Seq. ID No. 6637 | 1752277 | 1752259 | 1750973 | Seq. ID No. 6638 |
| 3145c | Seq. ID No. 6639 | 1752277 | 1752274 | 1750973 | Seq. ID No. 6640 |
| 3147a | Seq. ID No. 6641 | 125 | 1754054 | 1752510 | Seq. ID No. 6642 |
| 3147b | Seq. ID No. 6643 | 125 | 122 | 1752510 | Seq. ID No. 6644 |
| 3147c | Seq. ID No. 6645 | 125 | 83 | 1752510 | Seq. ID No. 6646 |
| 3149a | Seq. ID No. 6647 | 7 | 1754326 | 1754123 | Seq. ID No. 6648 |
| 3150a | Seq. ID No. 6649 | 1753936 | 1754086 | 1754331 | Seq. ID No. 6650 |

Listed in Table 3 are ORFs that have been identified using Signal P algorithm (i.e. Signal P value of “YES”) were considered to be surface exposed. 250 ORFs met all four of the criteria of the SignalP program for having a signal peptide (listed as a ‘YES’ in the SignalP column). A subset of 208 of these ORFs was also predicted by the HMM SignalP software to contain a signal peptide (Signal peptide). Of these 208 ORFs, the PSORT program predicted 21 ORFs to be localized to the periplasm (Peri); 29 ORFs to be localized to the outer membrane (OM); 153 ORFs to be localized to the cytoplasmic inner membrane (IM); and 5 ORFs to be localized to the cytoplasm. Of the 42 ORFs predicted by HMM SignalP to be non-secretory proteins (Non-secretory protein), 6 ORFs were predicted by PSORT to be localized to the periplasm and 7 ORFs were predicted by PSORT to be localized to the cytoplasm, 180 ORFs were predicted to be localized to the IM and 31 ORFs were predicted to be localized to the OM.

TABLE 3
SIGNALP: YES

| | DNA SEQ ID | Protein SEQ ID | | |
|---|---|---|---|---|
| ORF Number | Number | Number | HMMSignalP | PSORT |
| 51c | Seq. ID No. 89 | Seq. ID No. 90 | Non-secretory protein | IM |
| 69 | Seq. ID No. 127 | Seq. ID No. 128 | Non-secretory protein | Cyto |
| 73a | Seq. ID No. 131 | Seq. ID No. 132 | Signal peptide | Cyto |
| 73b | Seq. ID No. 133 | Seq. ID No. 134 | Signal peptide | Cyto |
| 87a | Seq. ID No. 161 | Seq. ID No. 162 | Signal peptide | OM |
| 87b | Seq. ID No. 163 | Seq. ID No. 164 | Signal peptide | IM |
| 109c | Seq. ID No. 213 | Seq. ID No. 214 | Signal peptide | IM |
| 109d | Seq. ID No. 215 | Seq. ID No. 216 | Signal peptide | IM |
| 114 | Seq. ID No. 225 | Seq. ID No. 226 | Signal peptide | OM |
| 129a | Seq. ID No. 249 | Seq. ID No. 250 | Signal peptide | IM |
| 129b | Seq. ID No. 251 | Seq. ID No. 252 | Signal peptide | IM |
| 145b | Seq. ID No. 287 | Seq. ID No. 288 | Signal peptide | IM |
| 145c | Seq. ID No. 289 | Seq. ID No. 290 | Signal peptide | IM |
| 179a | Seq. ID No. 369 | Seq. ID No. 370 | Non-secretory protein | IM |
| 179b | Seq. ID No. 371 | Seq. ID No. 372 | Non-secretory protein | IM |
| 180b | Seq. ID No. 375 | Seq. ID No. 376 | Signal peptide | IM |
| 183a | Seq. ID No. 377 | Seq. ID No. 378 | Non-secretory protein | IM |
| 245a | Seq. ID No. 497 | Seq. ID No. 498 | Signal peptide | IM |
| 246a | Seq. ID No. 503 | Seq. ID No. 504 | Signal peptide | IM |
| 246b | Seq. ID No. 505 | Seq. ID No. 506 | Signal peptide | IM |
| 255b | Seq. ID No. 527 | Seq. ID No. 528 | Signal peptide | IM |
| 264b | Seq. ID No. 549 | Seq. ID No. 550 | Signal peptide | IM |
| 277c | Seq. ID No. 575 | Seq. ID No. 576 | Signal peptide | OM |
| 297 | Seq. ID No. 617 | Seq. ID No. 618 | Signal peptide | Peri |
| 318a | Seq. ID No. 657 | Seq. ID No. 658 | Signal peptide | OM |
| 318b | Seq. ID No. 659 | Seq. ID No. 660 | Signal peptide | OM |
| 318c | Seq. ID No. 661 | Seq. ID No. 662 | Signal peptide | IM |
| 324 | Seq. ID No. 675 | Seq. ID No. 676 | Signal peptide | OM |
| 342a | Seq. ID No. 715 | Seq. ID No. 716 | Signal peptide | IM |
| 342b | Seq. ID No. 717 | Seq. ID No. 718 | Signal peptide | IM |
| 349b | Seq. ID No. 745 | Seq. ID No. 746 | Signal peptide | OM |
| 349c | Seq. ID No. 747 | Seq. ID No. 748 | Signal peptide | OM |
| 352a | Seq. ID No. 755 | Seq. ID No. 756 | Signal peptide | IM |
| 352b | Seq. ID No. 757 | Seq. ID No. 758 | Signal peptide | IM |
| 352c | Seq. ID No. 759 | Seq. ID No. 760 | Signal peptide | IM |
| 354a | Seq. ID No. 761 | Seq. ID No. 762 | Signal peptide | IM |
| 356c | Seq. ID No. 773 | Seq. ID No. 774 | Non-secretory protein | IM |
| 372d | Seq. ID No. 807 | Seq. ID No. 808 | Signal peptide | OM |
| 448 | Seq. ID No. 967 | Seq. ID No. 968 | Signal peptide | IM |
| 451a | Seq. ID No. 973 | Seq. ID No. 974 | Non-secretory protein | Peri |
| 451b | Seq. ID No. 975 | Seq. ID No. 976 | Non-secretory protein | Peri |
| 464b | Seq. ID No. 1013 | Seq. ID No. 1014 | Signal peptide | IM |
| 471a | Seq. ID No. 1025 | Seq. ID No. 1026 | Non-secretory protein | Cyto |
| 471b | Seq. ID No. 1027 | Seq. ID No. 1028 | Non-secretory protein | Cyto |
| 472f | Seq. ID No. 1039 | Seq. ID No. 1040 | Signal peptide | IM |
| 492 | Seq. ID No. 1075 | Seq. ID No. 1076 | Signal peptide | OM |
| 499a | Seq. ID No. 1089 | Seq. ID No. 1090 | Non-secretory protein | IM |
| 547a | Seq. ID No. 1195 | Seq. ID No. 1196 | Signal peptide | OM |
| 547b | Seq. ID No. 1197 | Seq. ID No. 1198 | Signal peptide | IM |
| 553c | Seq. ID No. 1207 | Seq. ID No. 1208 | Non-secretory protein | IM |
| 571 | Seq. ID No. 1239 | Seq. ID No. 1240 | Non-secretory protein | Peri |
| 576a | Seq. ID No. 1243 | Seq. ID No. 1244 | Signal peptide | Peri |
| 576b | Seq. ID No. 1245 | Seq. ID No. 1246 | Signal peptide | IM |
| 587c | Seq. ID No. 1269 | Seq. ID No. 1270 | Signal peptide | IM |
| 609a | Seq. ID No. 1315 | Seq. ID No. 1316 | Signal peptide | IM |
| 609b | Seq. ID No. 1317 | Seq. ID No. 1318 | Signal peptide | IM |
| 613d | Seq. ID No. 1331 | Seq. ID No. 1332 | Signal peptide | IM |
| 631b | Seq. ID No. 1365 | Seq. ID No. 1366 | Signal peptide | IM |
| 631c | Seq. ID No. 1367 | Seq. ID No. 1368 | Signal peptide | IM |
| 631d | Seq. ID No. 1369 | Seq. ID No. 1370 | Signal peptide | IM |
| 679a | Seq. ID No. 1445 | Seq. ID No. 1446 | Signal peptide | Cyto |
| 679b | Seq. ID No. 1447 | Seq. ID No. 1448 | Signal peptide | IM |
| 722a | Seq. ID No. 1513 | Seq. ID No. 1514 | Signal peptide | IM |
| 722c | Seq. ID No. 1517 | Seq. ID No. 1518 | Signal peptide | IM |
| 738a | Seq. ID No. 1545 | Seq. ID No. 1546 | Signal peptide | IM |
| 738c | Seq. ID No. 1549 | Seq. ID No. 1550 | Non-secretory protein | IM |
| 743b | Seq. ID No. 1561 | Seq. ID No. 1562 | Signal peptide | IM |
| 746a | Seq. ID No. 1569 | Seq. ID No. 1570 | Non-secretory protein | IM |
| 746b | Seq. ID No. 1571 | Seq. ID No. 1572 | Signal peptide | IM |
| 754b | Seq. ID No. 1585 | Seq. ID No. 1586 | Signal peptide | IM |
| 786b | Seq. ID No. 1643 | Seq. ID No. 1644 | Signal peptide | Peri |
| 789c | Seq. ID No. 1653 | Seq. ID No. 1654 | Signal peptide | IM |
| 794b | Seq. ID No. 1661 | Seq. ID No. 1662 | Signal peptide | OM |
| 794c | Seq. ID No. 1663 | Seq. ID No. 1664 | Signal peptide | OM |
| 826 | Seq. ID No. 1707 | Seq. ID No. 1708 | Signal peptide | IM |
| 837b | Seq. ID No. 1733 | Seq. ID No. 1734 | Signal peptide | IM |
| 843 | Seq. ID No. 1737 | Seq. ID No. 1738 | Signal peptide | IM |
| 853b | Seq. ID No. 1755 | Seq. ID No. 1756 | Signal peptide | IM |
| 859a | Seq. ID No. 1765 | Seq. ID No. 1766 | Signal peptide | IM |
| 859b | Seq. ID No. 1767 | Seq. ID No. 1768 | Signal peptide | IM |
| 862 | Seq. ID No. 1775 | Seq. ID No. 1776 | Non-secretory protein | Peri |
| 870 | Seq. ID No. 1791 | Seq. ID No. 1792 | Signal peptide | OM |
| 876 | Seq. ID No. 1805 | Seq. ID No. 1806 | Signal peptide | Peri |
| 931 | Seq. ID No. 1875 | Seq. ID No. 1876 | Signal peptide | IM |
| 977a | Seq. ID No. 1969 | Seq. ID No. 1970 | Signal peptide | OM |
| 981a | Seq. ID No. 1981 | Seq. ID No. 1982 | Signal peptide | IM |
| 1017c | Seq. ID No. 2053 | Seq. ID No. 2054 | Signal peptide | IM |
| 1017d | Seq. ID No. 2055 | Seq. ID No. 2056 | Signal peptide | IM |
| 1036a | Seq. ID No. 2087 | Seq. ID No. 2088 | Signal peptide | IM |
| 1036b | Seq. ID No. 2089 | Seq. ID No. 2090 | Signal peptide | IM |
| 1036c | Seq. ID No. 2091 | Seq. ID No. 2092 | Signal peptide | IM |
| 1044a | Seq. ID No. 2105 | Seq. ID No. 2106 | Signal peptide | IM |
| 1047b | Seq. ID No. 2115 | Seq. ID No. 2116 | Signal peptide | OM |
| 1065a | Seq. ID No. 2149 | Seq. ID No. 2150 | Signal peptide | Peri |
| 1065b | Seq. ID No. 2151 | Seq. ID No. 2152 | Signal peptide | Peri |
| 1067 | Seq. ID No. 2159 | Seq. ID No. 2160 | Signal peptide | OM |
| 1077d | Seq. ID No. 2177 | Seq. ID No. 2178 | Signal peptide | IM |
| 1141a | Seq. ID No. 2277 | Seq. ID No. 2278 | Non-secretory protein | Cyto |
| 1146c | Seq. ID No. 2293 | Seq. ID No. 2294 | Signal peptide | IM |
| 1155c | Seq. ID No. 2321 | Seq. ID No. 2322 | Signal peptide | IM |
| 1167a | Seq. ID No. 2347 | Seq. ID No. 2348 | Signal peptide | Peri |
| 1168 | Seq. ID No. 2351 | Seq. ID No. 2352 | Signal peptide | Peri |
| 1169a | Seq. ID No. 2353 | Seq. ID No. 2354 | Signal peptide | IM |
| 1172 | Seq. ID No. 2365 | Seq. ID No. 2366 | Signal peptide | IM |
| 1182b | Seq. ID No. 2395 | Seq. ID No. 2396 | Non-secretory protein | IM |
| 1192b | Seq. ID No. 2421 | Seq. ID No. 2422 | Signal peptide | IM |
| 1196a | Seq. ID No. 2425 | Seq. ID No. 2426 | Signal peptide | Peri |
| 1196b | Seq. ID No. 2427 | Seq. ID No. 2428 | Signal peptide | Peri |
| 1197 | Seq. ID No. 2429 | Seq. ID No. 2430 | Signal peptide | IM |
| 1207 | Seq. ID No. 2449 | Seq. ID No. 2450 | Signal peptide | OM |
| 1212 | Seq. ID No. 2459 | Seq. ID No. 2460 | Signal peptide | IM |
| 1237 | Seq. ID No. 2525 | Seq. ID No. 2526 | Signal peptide | OM |
| 1241a | Seq. ID No. 2531 | Seq. ID No. 2532 | Signal peptide | IM |
| 1268b | Seq. ID No. 2607 | Seq. ID No. 2608 | Signal peptide | IM |
| 1288 | Seq. ID No. 2651 | Seq. ID No. 2652 | Signal peptide | IM |
| 1308a | Seq. ID No. 2679 | Seq. ID No. 2680 | Signal peptide | IM |
| 1315 | Seq. ID No. 2693 | Seq. ID No. 2694 | Signal peptide | IM |
| 1318 | Seq. ID No. 2699 | Seq. ID No. 2700 | Signal peptide | IM |
| 1319b | Seq. ID No. 2703 | Seq. ID No. 2704 | Signal peptide | IM |
| 1348 | Seq. ID No. 2753 | Seq. ID No. 2754 | Non-secretory protein | IM |
| 1366a | Seq. ID No. 2785 | Seq. ID No. 2786 | Signal peptide | Peri |
| 1381b | Seq. ID No. 2813 | Seq. ID No. 2814 | Signal peptide | IM |
| 1387a | Seq. ID No. 2829 | Seq. ID No. 2830 | Non-secretory protein | OM |
| 1393a | Seq. ID No. 2835 | Seq. ID No. 2836 | Signal peptide | Peri |
| 1393d | Seq. ID No. 2841 | Seq. ID No. 2842 | Signal peptide | IM |
| 1412a | Seq. ID No. 2879 | Seq. ID No. 2880 | Non-secretory protein | IM |
| 1412b | Seq. ID No. 2881 | Seq. ID No. 2882 | Signal peptide | IM |
| 1438 | Seq. ID No. 2935 | Seq. ID No. 2936 | Signal peptide | OM |
| 1439a | Seq. ID No. 2937 | Seq. ID No. 2938 | Signal peptide | IM |
| 1440b | Seq. ID No. 2949 | Seq. ID No. 2950 | Signal peptide | IM |
| 1446a | Seq. ID No. 2965 | Seq. ID No. 2966 | Non-secretory protein | IM |
| 1446b | Seq. ID No. 2967 | Seq. ID No. 2968 | Non-secretory protein | IM |
| 1448 | Seq. ID No. 2973 | Seq. ID No. 2974 | Signal peptide | IM |
| 1463a | Seq. ID No. 3007 | Seq. ID No. 3008 | Signal peptide | IM |
| 1463b | Seq. ID No. 3009 | Seq. ID No. 3010 | Signal peptide | IM |
| 1463c | Seq. ID No. 3011 | Seq. ID No. 3012 | Signal peptide | IM |
| 1470a | Seq. ID No. 3029 | Seq. ID No. 3030 | Signal peptide | IM |
| 1470b | Seq. ID No. 3031 | Seq. ID No. 3032 | Signal peptide | IM |
| 1503a | Seq. ID No. 3109 | Seq. ID No. 3110 | Non-secretory protein | IM |
| 1503b | Seq. ID No. 3111 | Seq. ID No. 3112 | Non-secretory protein | IM |
| 1559a | Seq. ID No. 3219 | Seq. ID No. 3220 | Signal peptide | IM |
| 1563 | Seq. ID No. 3223 | Seq. ID No. 3224 | Non-secretory protein | Cyto |
| 1584 | Seq. ID No. 3273 | Seq. ID No. 3274 | Non-secretory protein | Peri |
| 1594a | Seq. ID No. 3297 | Seq. ID No. 3298 | Signal peptide | IM |
| 1612 | Seq. ID No. 3331 | Seq. ID No. 3332 | Signal peptide | IM |
| 1616b | Seq. ID No. 3335 | Seq. ID No. 3336 | Signal peptide | IM |
| 1622a | Seq. ID No. 3341 | Seq. ID No. 3342 | Non-secretory protein | Cyto |
| 1624 | Seq. ID No. 3349 | Seq. ID No. 3350 | Signal peptide | Peri |
| 1625b | Seq. ID No. 3353 | Seq. ID No. 3354 | Signal peptide | IM |
| 1625c | Seq. ID No. 3355 | Seq. ID No. 3356 | Signal peptide | IM |
| 1634c | Seq. ID No. 3377 | Seq. ID No. 3378 | Signal peptide | IM |
| 1646b | Seq. ID No. 3391 | Seq. ID No. 3392 | Signal peptide | IM |
| 1659c | Seq. ID No. 3425 | Seq. ID No. 3426 | Signal peptide | IM |
| 1673a | Seq. ID No. 3449 | Seq. ID No. 3450 | Signal peptide | IM |
| 1688b | Seq. ID No. 3493 | Seq. ID No. 3494 | Signal peptide | IM |
| 1718b | Seq. ID No. 3573 | Seq. ID No. 3574 | Signal peptide | Cyto |
| 1743 | Seq. ID No. 3611 | Seq. ID No. 3612 | Signal peptide | IM |
| 1747 | Seq. ID No. 3619 | Seq. ID No. 3620 | Signal peptide | IM |
| 1784b | Seq. ID No. 3689 | Seq. ID No. 3690 | Signal peptide | IM |
| 1808a | Seq. ID No. 3753 | Seq. ID No. 3754 | Signal peptide | IM |
| 1808b | Seq. ID No. 3755 | Seq. ID No. 3756 | Signal peptide | IM |
| 1809c | Seq. ID No. 3765 | Seq. ID No. 3766 | Signal peptide | IM |
| 1812a | Seq. ID No. 3781 | Seq. ID No. 3782 | Signal peptide | IM |
| 1812b | Seq. ID No. 3783 | Seq. ID No. 3784 | Signal peptide | IM |
| 1842b | Seq. ID No. 3851 | Seq. ID No. 3852 | Signal peptide | IM |
| 1862a | Seq. ID No. 3903 | Seq. ID No. 3904 | Signal peptide | IM |
| 1919a | Seq. ID No. 4005 | Seq. ID No. 4006 | Signal peptide | Peri |
| 1919b | Seq. ID No. 4007 | Seq. ID No. 4008 | Signal peptide | IM |
| 1931b | Seq. ID No. 4027 | Seq. ID No. 4028 | Signal peptide | IM |
| 1946b | Seq. ID No. 4073 | Seq. ID No. 4074 | Signal peptide | IM |
| 1973 | Seq. ID No. 4137 | Seq. ID No. 4138 | Signal peptide | OM |
| 1977a | Seq. ID No. 4141 | Seq. ID No. 4142 | Signal peptide | OM |
| 1977b | Seq. ID No. 4143 | Seq. ID No. 4144 | Signal peptide | OM |
| 1977c | Seq. ID No. 4145 | Seq. ID No. 4146 | Signal peptide | OM |
| 2016c | Seq. ID No. 4225 | Seq. ID No. 4226 | Signal peptide | IM |
| 2074b | Seq. ID No. 4343 | Seq. ID No. 4344 | Signal peptide | OM |
| 2096a | Seq. ID No. 4385 | Seq. ID No. 4386 | Signal peptide | IM |
| 2101 | Seq. ID No. 4399 | Seq. ID No. 4400 | Signal peptide | Peri |
| 2140a | Seq. ID No. 4469 | Seq. ID No. 4470 | Signal peptide | IM |
| 2147a | Seq. ID No. 4483 | Seq. ID No. 4484 | Signal peptide | IM |
| 2150a | Seq. ID No. 4493 | Seq. ID No. 4494 | Signal peptide | IM |
| 2150b | Seq. ID No. 4495 | Seq. ID No. 4496 | Signal peptide | IM |
| 2165 | Seq. ID No. 4533 | Seq. ID No. 4534 | Signal peptide | IM |
| 2245 | Seq. ID No. 4699 | Seq. ID No. 4700 | Non-secretory protein | OM |
| 2253b | Seq. ID No. 4709 | Seq. ID No. 4710 | Signal peptide | IM |
| 2253c | Seq. ID No. 4711 | Seq. ID No. 4712 | Signal peptide | IM |
| 2264a | Seq. ID No. 4735 | Seq. ID No. 4736 | Non-secretory protein | Peri |
| 2308 | Seq. ID No. 4827 | Seq. ID No. 4828 | Signal peptide | OM |
| 2329b | Seq. ID No. 4871 | Seq. ID No. 4872 | Signal peptide | IM |
| 2329c | Seq. ID No. 4873 | Seq. ID No. 4874 | Signal peptide | IM |
| 2331 | Seq. ID No. 4879 | Seq. ID No. 4880 | Non-secretory protein | IM |
| 2358 | Seq. ID No. 4921 | Seq. ID No. 4922 | Signal peptide | IM |
| 2386b | Seq. ID No. 4981 | Seq. ID No. 4982 | Non-secretory protein | IM |
| 2453 | Seq. ID No. 5115 | Seq. ID No. 5116 | Signal peptide | OM |
| 2518a | Seq. ID No. 5267 | Seq. ID No. 5268 | Signal peptide | Cyto |
| 2550c | Seq. ID No. 5341 | Seq. ID No. 5342 | Signal peptide | IM |
| 2567b | Seq. ID No. 5387 | Seq. ID No. 5388 | Signal peptide | IM |
| 2616d | Seq. ID No. 5465 | Seq. ID No. 5466 | Signal peptide | IM |
| 2616e | Seq. ID No. 5467 | Seq. ID No. 5468 | Non-secretory protein | IM |
| 2618a | Seq. ID No. 5479 | Seq. ID No. 5480 | Non-secretory protein | IM |
| 2625a | Seq. ID No. 5497 | Seq. ID No. 5498 | Signal peptide | Peri |
| 2629b | Seq. ID No. 5513 | Seq. ID No. 5514 | Signal peptide | OM |
| 2648a | Seq. ID No. 5543 | Seq. ID No. 5544 | Signal peptide | IM |
| 2648b | Seq. ID No. 5545 | Seq. ID No. 5546 | Signal peptide | IM |
| 2659b | Seq. ID No. 5567 | Seq. ID No. 5568 | Non-secretory protein | IM |
| 2669a | Seq. ID No. 5587 | Seq. ID No. 5588 | Signal peptide | Peri |
| 2669b | Seq. ID No. 5589 | Seq. ID No. 5590 | Signal peptide | IM |
| 2669c | Seq. ID No. 5591 | Seq. ID No. 5592 | Signal peptide | IM |
| 2679b | Seq. ID No. 5611 | Seq. ID No. 5612 | Signal peptide | IM |
| 2680c | Seq. ID No. 5621 | Seq. ID No. 5622 | Signal peptide | IM |
| 2689b | Seq. ID No. 5641 | Seq. ID No. 5642 | Signal peptide | Peri |
| 2718a | Seq. ID No. 5681 | Seq. ID No. 5682 | Signal peptide | IM |
| 2734a | Seq. ID No. 5719 | Seq. ID No. 5720 | Signal peptide | IM |
| 2749e | Seq. ID No. 5771 | Seq. ID No. 5772 | Signal peptide | IM |
| 2757b | Seq. ID No. 5783 | Seq. ID No. 5784 | Signal peptide | IM |
| 2801b | Seq. ID No. 5895 | Seq. ID No. 5896 | Signal peptide | IM |
| 2805a | Seq. ID No. 5899 | Seq. ID No. 5900 | Signal peptide | IM |
| 2805b | Seq. ID No. 5901 | Seq. ID No. 5902 | Signal peptide | IM |
| 2818d | Seq. ID No. 5927 | Seq. ID No. 5928 | Signal peptide | IM |
| 2818e | Seq. ID No. 5929 | Seq. ID No. 5930 | Signal peptide | IM |
| 2822a | Seq. ID No. 5939 | Seq. ID No. 5940 | Signal peptide | IM |
| 2852 | Seq. ID No. 5987 | Seq. ID No. 5988 | Non-secretory protein | IM |
| 2862 | Seq. ID No. 6013 | Seq. ID No. 6014 | Non-secretory protein | Cyto |
| 2900a | Seq. ID No. 6087 | Seq. ID No. 6088 | Signal peptide | Peri |
| 2900b | Seq. ID No. 6089 | Seq. ID No. 6090 | Signal peptide | Peri |
| 2901a | Seq. ID No. 6091 | Seq. ID No. 6092 | Signal peptide | IM |
| 2901b | Seq. ID No. 6093 | Seq. ID No. 6094 | Non-secretory protein | IM |
| 2902 | Seq. ID No. 6095 | Seq. ID No. 6096 | Signal peptide | IM |
| 2906 | Seq. ID No. 6103 | Seq. ID No. 6104 | Signal peptide | IM |
| 2933b | Seq. ID No. 6169 | Seq. ID No. 6170 | Signal peptide | IM |
| 2941b | Seq. ID No. 6185 | Seq. ID No. 6186 | Signal peptide | IM |
| 2941c | Seq. ID No. 6187 | Seq. ID No. 6188 | Signal peptide | IM |
| 2943d | Seq. ID No. 6195 | Seq. ID No. 6196 | Non-secretory protein | IM |
| 2944b | Seq. ID No. 6197 | Seq. ID No. 6198 | Signal peptide | IM |
| 2948a | Seq. ID No. 6203 | Seq. ID No. 6204 | Signal peptide | IM |
| 2948b | Seq. ID No. 6205 | Seq. ID No. 6206 | Signal peptide | IM |
| 2955a | Seq. ID No. 6221 | Seq. ID No. 6222 | Signal peptide | IM |
| 2955b | Seq. ID No. 6223 | Seq. ID No. 6224 | Signal peptide | IM |
| 2955e | Seq. ID No. 6229 | Seq. ID No. 6230 | Signal peptide | IM |
| 2980b | Seq. ID No. 6291 | Seq. ID No. 6292 | Non-secretory protein | IM |
| 3037a | Seq. ID No. 6415 | Seq. ID No. 6416 | Signal peptide | OM |
| 3037b | Seq. ID No. 6417 | Seq. ID No. 6418 | Non-secretory protein | IM |
| 3064b | Seq. ID No. 6473 | Seq. ID No. 6474 | Signal peptide | Peri |
| 3079 | Seq. ID No. 6507 | Seq. ID No. 6508 | Non-secretory protein | IM |
| 3096b | Seq. ID No. 6543 | Seq. ID No. 6544 | Signal peptide | IM |
| 3119b | Seq. ID No. 6595 | Seq. ID No. 6596 | Signal peptide | IM |
| 3119c | Seq. ID No. 6597 | Seq. ID No. 6598 | Signal peptide | IM |
| 3119d | Seq. ID No. 6599 | Seq. ID No. 6600 | Signal peptide | IM |
| 3119e | Seq. ID No. 6601 | Seq. ID No. 6602 | Signal peptide | IM |
| 3119f | Seq. ID No. 6603 | Seq. ID No. 6604 | Signal peptide | IM |

Listed in Table 4 are all 526 ORFs of the SignalP program that met only three of the criteria of having a signal peptide (listed as a ‘yes’ in the SignalP column). A subset of 359 of these ORFs was also predicted by the HMM SignalP software to contain a signal peptide. Of these 359 ORFs, the PSORT program predicted 24 ORFs to be localized to the periplasm (Peri); 44 ORFs to be localized to the outer membrane (OM); 267 ORFs to be localized to the cytoplasmic membrane (IM); and 24 ORFs to be localized to the cytoplasm. Of the 167 ORFs predicted by HMM SignalP to be Non-secretory proteins, PSORT predicted 6 ORFs to be localized to the periplasm; 1 ORF to be localized to the outer membrane; 122 ORFs to be localized to the cytoplasmic membrane; and 38 ORFs to be localized to the cytoplasm. Also listed for all ORFs is the predicted number of transmembrane, a-helical hydrophobic domains predicted by the TopPred2 software (listed in the MSD column). If no value is listed, none has yet been determined. (The same is also the case for Tables 5 and 6).

TABLE 4
SIGNALP: YES

| ORF | DNA SEQ ID | Protein SEQ ID | | | |
|---|---|---|---|---|---|
| Number | Number | Number | HMMSignalP | PSORT | MSD |
| 4a | Seq. ID No. 7 | Seq. ID No. 8 | Signal peptide | IM | 4 |
| 4c | Seq. ID No. 11 | Seq. ID No. 12 | Non-secretory protein | IM | 12 |
| 16 | Seq. ID No. 23 | Seq. ID No. 24 | Non-secretory protein | IM | 0 |
| 38a | Seq. ID No. 45 | Seq. ID No. 46 | Signal peptide | OM | 1 |
| 38b | Seq. ID No. 47 | Seq. ID No. 48 | Signal peptide | OM | 1 |
| 40 | Seq. ID No. 53 | Seq. ID No. 54 | Signal peptide | IM | 1 |
| 41c | Seq. ID No. 59 | Seq. ID No. 60 | Non-secretory protein | IM | 1 |
| 44a | Seq. ID No. 65 | Seq. ID No. 66 | Signal peptide | IM | 2 |
| 44b | Seq. ID No. 67 | Seq. ID No. 68 | Signal peptide | IM | 2 |
| 51b | Seq. ID No. 87 | Seq. ID No. 88 | Signal peptide | IM | 9 |
| 55 | Seq. ID No. 91 | Seq. ID No. 92 | Non-secretory protein | IM | 1 |
| 79 | Seq. ID No. 139 | Seq. ID No. 140 | Non-secretory protein | IM | 2 |
| 83 | Seq. ID No. 151 | Seq. ID No. 152 | Non-secretory protein | IM | 2 |
| 86 | Seq. ID No. 159 | Seq. ID No. 160 | Signal peptide | Peri | 1 |
| 87c | Seq. ID No. 165 | Seq. ID No. 166 | Non-secretory protein | IM | 2 |
| 89 | Seq. ID No. 169 | Seq. ID No. 170 | Non-secretory protein | IM | 2 |
| 94 | Seq. ID No. 185 | Seq. ID No. 186 | Non-secretory protein | IM | 2 |
| 109b | Seq. ID No. 211 | Seq. ID No. 212 | Signal peptide | IM | 7 |
| 111a | Seq. ID No. 217 | Seq. ID No. 218 | Signal peptide | IM | 6 |
| 124a | Seq. ID No. 235 | Seq. ID No. 236 | Signal peptide | IM | 1 |
| 124b | Seq. ID No. 237 | Seq. ID No. 238 | Signal peptide | IM | 3 |
| 124c | Seq. ID No. 239 | Seq. ID No. 240 | Non-secretory protein | IM | 5 |
| 138a | Seq. ID No. 271 | Seq. ID No. 272 | Signal peptide | IM | 6 |
| 145a | Seq. ID No. 285 | Seq. ID No. 286 | Signal peptide | IM | 3 |
| 145d | Seq. ID No. 291 | Seq. ID No. 292 | Non-secretory protein | IM | 12 |
| 162a | Seq. ID No. 327 | Seq. ID No. 328 | Signal peptide | IM | 2 |
| 170 | Seq. ID No. 349 | Seq. ID No. 350 | Non-secretory protein | IM | 1 |
| 187a | Seq. ID No. 385 | Seq. ID No. 386 | Non-secretory protein | IM | 1 |
| 187b | Seq. ID No. 387 | Seq. ID No. 388 | Non-secretory protein | IM | 1 |
| 196 | Seq. ID No. 407 | Seq. ID No. 408 | Non-secretory protein | Cyto | 1 |
| 205 | Seq. ID No. 427 | Seq. ID No. 428 | Non-secretory protein | Cyto | |
| 207 | Seq. ID No. 431 | Seq. ID No. 432 | Non-secretory protein | IM | 0 |
| 223a | Seq. ID No. 455 | Seq. ID No. 456 | Signal peptide | Cyto | 1 |
| 223b | Seq. ID No. 457 | Seq. ID No. 458 | Signal peptide | Cyto | 1 |
| 241 | Seq. ID No. 495 | Seq. ID No. 496 | Signal peptide | IM | 1 |
| 245b | Seq. ID No. 499 | Seq. ID No. 500 | Signal peptide | IM | 10 |
| 248b | Seq. ID No. 511 | Seq. ID No. 512 | Signal peptide | IM | 9 |
| 249a | Seq. ID No. 513 | Seq. ID No. 514 | Signal peptide | OM | 1 |
| 249b | Seq. ID No. 515 | Seq. ID No. 516 | Signal peptide | IM | 1 |
| 255a | Seq. ID No. 525 | Seq. ID No. 526 | Signal peptide | IM | 6 |
| 263a | Seq. ID No. 545 | Seq. ID No. 546 | Signal peptide | OM | 1 |
| 263b | Seq. ID No. 547 | Seq. ID No. 548 | Signal peptide | IM | 1 |
| 268 | Seq. ID No. 557 | Seq. ID No. 558 | Signal peptide | Cyto | 1 |
| 273b | Seq. ID No. 569 | Seq. ID No. 570 | Signal peptide | IM | 12 |
| 282 | Seq. ID No. 589 | Seq. ID No. 590 | Non-secretory protein | Cyto | |
| 283a | Seq. ID No. 591 | Seq. ID No. 592 | Signal peptide | OM | 1 |
| 283b | Seq. ID No. 593 | Seq. ID No. 594 | Signal peptide | OM | 1 |
| 284a | Seq. ID No. 595 | Seq. ID No. 596 | Non-secretory protein | IM | 5 |
| 290b | Seq. ID No. 609 | Seq. ID No. 610 | Non-secretory protein | IM | |
| 311 | Seq. ID No. 637 | Seq. ID No. 638 | Signal peptide | IM | 6 |
| 313a | Seq. ID No. 639 | Seq. ID No. 640 | Signal peptide | IM | 2 |
| 313b | Seq. ID No. 641 | Seq. ID No. 642 | Signal peptide | IM | 3 |
| 317a | Seq. ID No. 653 | Seq. ID No. 654 | Signal peptide | IM | 2 |
| 321 | Seq. ID No. 669 | Seq. ID No. 670 | Signal peptide | OM | 1 |
| 327b | Seq. ID No. 679 | Seq. ID No. 680 | Signal peptide | IM | 5 |
| 342c | Seq. ID No. 719 | Seq. ID No. 720 | Signal peptide | IM | 4 |
| 342e | Seq. ID No. 723 | Seq. ID No. 724 | Signal peptide | IM | 10 |
| 346b | Seq. ID No. 739 | Seq. ID No. 740 | Non-secretory protein | Cyto | 0 |
| 350a | Seq. ID No. 749 | Seq. ID No. 750 | Signal peptide | IM | 6 |
| 350b | Seq. ID No. 751 | Seq. ID No. 752 | Signal peptide | IM | 6 |
| 354b | Seq. ID No. 763 | Seq. ID No. 764 | Signal peptide | IM | 6 |
| 354c | Seq. ID No. 765 | Seq. ID No. 766 | Signal peptide | IM | 6 |
| 356a | Seq. ID No. 769 | Seq. ID No. 770 | Signal peptide | IM | 3 |
| 358a | Seq. ID No. 777 | Seq. ID No. 778 | Signal peptide | IM | 3 |
| 358b | Seq. ID No. 779 | Seq. ID No. 780 | Signal peptide | IM | 10 |
| 358c | Seq. ID No. 781 | Seq. ID No. 782 | Signal peptide | IM | 10 |
| 382b | Seq. ID No. 819 | Seq. ID No. 820 | Signal peptide | OM | 1 |
| 418b | Seq. ID No. 895 | Seq. ID No. 896 | Non-secretory protein | IM | 7 |
| 420a | Seq. ID No. 903 | Seq. ID No. 904 | Non-secretory protein | IM | 1 |
| 420b | Seq. ID No. 905 | Seq. ID No. 906 | Non-secretory protein | IM | 1 |
| 422 | Seq. ID No. 909 | Seq. ID No. 910 | Non-secretory protein | Cyto | |
| 431 | Seq. ID No. 935 | Seq. ID No. 936 | Signal peptide | OM | 1 |
| 434 | Seq. ID No. 937 | Seq. ID No. 938 | Signal peptide | IM | 2 |
| 440 | Seq. ID No. 951 | Seq. ID No. 952 | Non-secretory protein | IM | 2 |
| 441b | Seq. ID No. 955 | Seq. ID No. 956 | Signal peptide | IM | 2 |
| 452b | Seq. ID No. 979 | Seq. ID No. 980 | Signal peptide | IM | 4 |
| 452c | Seq. ID No. 981 | Seq. ID No. 982 | Signal peptide | IM | 4 |
| 452d | Seq. ID No. 983 | Seq. ID No. 984 | Signal peptide | IM | 4 |
| 452e | Seq. ID No. 985 | Seq. ID No. 986 | Signal peptide | IM | 4 |
| 462 | Seq. ID No. 1005 | Seq. ID No. 1006 | Signal peptide | Peri | 1 |
| 463a | Seq. ID No. 1007 | Seq. ID No. 1008 | Signal peptide | Peri | 1 |
| 463b | Seq. ID No. 1009 | Seq. ID No. 1010 | Signal peptide | Peri | 1 |
| 464c | Seq. ID No. 1015 | Seq. ID No. 1016 | Signal peptide | IM | 11 |
| 465b | Seq. ID No. 1019 | Seq. ID No. 1020 | Signal peptide | OM | 3 |
| 472a | Seq. ID No. 1029 | Seq. ID No. 1030 | Signal peptide | IM | 4 |
| 472b | Seq. ID No. 1031 | Seq. ID No. 1032 | Signal peptide | IM | 5 |
| 477b | Seq. ID No. 1047 | Seq. ID No. 1048 | Non-secretory protein | Cyto | 0 |
| 499b | Seq. ID No. 1091 | Seq. ID No. 1092 | Non-secretory protein | IM | 2 |
| 510 | Seq. ID No. 1111 | Seq. ID No. 1112 | Non-secretory protein | Cyto | |
| 524b | Seq. ID No. 1137 | Seq. ID No. 1138 | Signal peptide | IM | 6 |
| 530a | Seq. ID No. 1151 | Seq. ID No. 1152 | Signal peptide | IM | |
| 530b | Seq. ID No. 1153 | Seq. ID No. 1154 | Non-secretory protein | Peri | |
| 541c | Seq. ID No. 1169 | Seq. ID No. 1170 | Signal peptide | IM | 5 |
| 542 | Seq. ID No. 1173 | Seq. ID No. 1174 | Signal peptide | IM | 1 |
| 545a | Seq. ID No. 1183 | Seq. ID No. 1184 | Signal peptide | IM | 3 |
| 546b | Seq. ID No. 1191 | Seq. ID No. 1192 | Non-secretory protein | IM | 1 |
| 546c | Seq. ID No. 1193 | Seq. ID No. 1194 | Non-secretory protein | IM | 1 |
| 556 | Seq. ID No. 1213 | Seq. ID No. 1214 | Signal peptide | IM | 2 |
| 561 | Seq. ID No. 1223 | Seq. ID No. 1224 | Signal peptide | IM | 5 |
| 564b | Seq. ID No. 1229 | Seq. ID No. 1230 | Signal peptide | IM | 4 |
| 578a | Seq. ID No. 1247 | Seq. ID No. 1248 | Non-secretory protein | Cyto | 0 |
| 585b | Seq. ID No. 1259 | Seq. ID No. 1260 | Signal peptide | Cyto | 0 |
| 595a | Seq. ID No. 1283 | Seq. ID No. 1284 | Signal peptide | IM | 2 |
| 603a | Seq. ID No. 1307 | Seq. ID No. 1308 | Non-secretory protein | Cyto | 1 |
| 610a | Seq. ID No. 1319 | Seq. ID No. 1320 | Signal peptide | IM | 1 |
| 610b | Seq. ID No. 1321 | Seq. ID No. 1322 | Non-secretory protein | IM | 1 |
| 613b | Seq. ID No. 1327 | Seq. ID No. 1328 | Signal peptide | IM | 7 |
| 619a | Seq. ID No. 1341 | Seq. ID No. 1342 | Signal peptide | Cyto | 1 |
| 619b | Seq. ID No. 1343 | Seq. ID No. 1344 | Signal peptide | Cyto | 1 |
| 623 | Seq. ID No. 1347 | Seq. ID No. 1348 | Signal peptide | Peri | 1 |
| 640 | Seq. ID No. 1389 | Seq. ID No. 1390 | Non-secretory protein | Cyto | 0 |
| 645 | Seq. ID No. 1401 | Seq. ID No. 1402 | Signal peptide | Peri | 1 |
| 665 | Seq. ID No. 1429 | Seq. ID No. 1430 | Signal peptide | IM | 3 |
| 699b | Seq. ID No. 1479 | Seq. ID No. 1480 | Non-secretory protein | IM | 1 |
| 739 | Seq. ID No. 1551 | Seq. ID No. 1552 | Signal peptide | IM | 2 |
| 743a | Seq. ID No. 1559 | Seq. ID No. 1560 | Signal peptide | IM | 2 |
| 749 | Seq. ID No. 1579 | Seq. ID No. 1580 | Signal peptide | IM | 1 |
| 754c | Seq. ID No. 1587 | Seq. ID No. 1588 | Non-secretory protein | IM | 9 |
| 772a | Seq. ID No. 1621 | Seq. ID No. 1622 | Signal peptide | IM | 5 |
| 775 | Seq. ID No. 1627 | Seq. ID No. 1628 | Non-secretory protein | IM | 7 |
| 779 | Seq. ID No. 1633 | Seq. ID No. 1634 | Signal peptide | Peri | 2 |
| 788a | Seq. ID No. 1645 | Seq. ID No. 1646 | Signal peptide | IM | 4 |
| 788b | Seq. ID No. 1647 | Seq. ID No. 1648 | Signal peptide | IM | 6 |
| 789a | Seq. ID No. 1649 | Seq. ID No. 1650 | Signal peptide | IM | 4 |
| 791 | Seq. ID No. 1655 | Seq. ID No. 1656 | Non-secretory protein | Cyto | |
| 794d | Seq. ID No. 1665 | Seq. ID No. 1666 | Signal peptide | OM | 0 |
| 795 | Seq. ID No. 1667 | Seq. ID No. 1668 | Non-secretory protein | Cyto | |
| 799a | Seq. ID No. 1671 | Seq. ID No. 1672 | Signal peptide | IM | 2 |
| 799d | Seq. ID No. 1677 | Seq. ID No. 1678 | Signal peptide | IM | 9 |
| 799e | Seq. ID No. 1679 | Seq. ID No. 1680 | Signal peptide | IM | 10 |
| 799f | Seq. ID No. 1681 | Seq. ID No. 1682 | Signal peptide | IM | 10 |
| 809b | Seq. ID No. 1695 | Seq. ID No. 1696 | Non-secretory protein | IM | 4 |
| 820a | Seq. ID No. 1703 | Seq. ID No. 1704 | Non-secretory protein | IM | 0 |
| 820b | Seq. ID No. 1705 | Seq. ID No. 1706 | Non-secretory protein | IM | 0 |
| 837a | Seq. ID No. 1731 | Seq. ID No. 1732 | Signal peptide | IM | 2 |
| 853a | Seq. ID No. 1753 | Seq. ID No. 1754 | Signal peptide | IM | 3 |
| 854 | Seq. ID No. 1759 | Seq. ID No. 1760 | Non-secretory protein | IM | 2 |
| 857 | Seq. ID No. 1763 | Seq. ID No. 1764 | Non-secretory protein | Peri | 1 |
| 868a | Seq. ID No. 1787 | Seq. ID No. 1788 | Signal peptide | IM | 3 |
| 873c | Seq. ID No. 1797 | Seq. ID No. 1798 | Signal peptide | OM | 1 |
| 886 | Seq. ID No. 1821 | Seq. ID No. 1822 | Signal peptide | OM | 1 |
| 887 | Seq. ID No. 1823 | Seq. ID No. 1824 | Signal peptide | IM | 1 |
| 904 | Seq. ID No. 1833 | Seq. ID No. 1834 | Non-secretory protein | Peri | |
| 908a | Seq. ID No. 1839 | Seq. ID No. 1840 | Signal peptide | IM | 6 |
| 908b | Seq. ID No. 1841 | Seq. ID No. 1842 | Non-secretory protein | IM | 9 |
| 908c | Seq. ID No. 1843 | Seq. ID No. 1844 | Non-secretory protein | IM | 9 |
| 939b | Seq. ID No. 1893 | Seq. ID No. 1894 | Signal peptide | IM | 10 |
| 951a | Seq. ID No. 1917 | Seq. ID No. 1918 | Signal peptide | OM | 0 |
| 951b | Seq. ID No. 1919 | Seq. ID No. 1920 | Signal peptide | IM | 0 |
| 977b | Seq. ID No. 1971 | Seq. ID No. 1972 | Non-secretory protein | Cyto | 1 |
| 987 | Seq. ID No. 1991 | Seq. ID No. 1992 | Signal peptide | IM | 5 |
| 998a | Seq. ID No. 2013 | Seq. ID No. 2014 | Signal peptide | IM | 5 |
| 998b | Seq. ID No. 2015 | Seq. ID No. 2016 | Non-secretory protein | IM | 5 |
| 999a | Seq. ID No. 2017 | Seq. ID No. 2018 | Signal peptide | IM | 2 |
| 999b | Seq. ID No. 2019 | Seq. ID No. 2020 | Signal peptide | IM | 2 |
| 1006a | Seq. ID No. 2033 | Seq. ID No. 2034 | Signal peptide | Peri | 1 |
| 1006b | Seq. ID No. 2035 | Seq. ID No. 2036 | Signal peptide | Cyto | 1 |
| 1017a | Seq. ID No. 2049 | Seq. ID No. 2050 | Signal peptide | IM | 4 |
| 1035a | Seq. ID No. 2081 | Seq. ID No. 2082 | Non-secretory protein | IM | 1 |
| 1035b | Seq. ID No. 2083 | Seq. ID No. 2084 | Non-secretory protein | IM | 1 |
| 1035c | Seq. ID No. 2085 | Seq. ID No. 2086 | Non-secretory protein | IM | 1 |
| 1044d | Seq. ID No. 2111 | Seq. ID No. 2112 | Non-secretory protein | IM | 3 |
| 1047a | Seq. ID No. 2113 | Seq. ID No. 2114 | Signal peptide | OM | 1 |
| 1050b | Seq. ID No. 2123 | Seq. ID No. 2124 | Signal peptide | IM | 7 |
| 1050c | Seq. ID No. 2125 | Seq. ID No. 2126 | Signal peptide | IM | 7 |
| 1050d | Seq. ID No. 2127 | Seq. ID No. 2128 | Signal peptide | IM | 7 |
| 1056a | Seq. ID No. 2131 | Seq. ID No. 2132 | Non-secretory protein | Cyto | |
| 1056b | Seq. ID No. 2133 | Seq. ID No. 2134 | Non-secretory protein | Cyto | |
| 1065c | Seq. ID No. 2153 | Seq. ID No. 2154 | Non-secretory protein | IM | 1 |
| 1069 | Seq. ID No. 2161 | Seq. ID No. 2162 | Non-secretory protein | IM | 1 |
| 1077a | Seq. ID No. 2171 | Seq. ID No. 2172 | Signal peptide | IM | 3 |
| 1077c | Seq. ID No. 2175 | Seq. ID No. 2176 | Non-secretory protein | IM | 12 |
| 1077e | Seq. ID No. 2179 | Seq. ID No. 2180 | Non-secretory protein | IM | 15 |
| 1081a | Seq. ID No. 2187 | Seq. ID No. 2188 | Signal peptide | Peri | 1 |
| 1091a | Seq. ID No. 2201 | Seq. ID No. 2202 | Non-secretory protein | Cyto | |
| 1091b | Seq. ID No. 2203 | Seq. ID No. 2204 | Non-secretory protein | Peri | |
| 1098b | Seq. ID No. 2221 | Seq. ID No. 2222 | Non-secretory protein | IM | 2 |
| 1111a | Seq. ID No. 2237 | Seq. ID No. 2238 | Signal peptide | IM | 6 |
| 1126b | Seq. ID No. 2249 | Seq. ID No. 2250 | Signal peptide | IM | 4 |
| 1126c | Seq. ID No. 2251 | Seq. ID No. 2252 | Signal peptide | IM | 4 |
| 1128 | Seq. ID No. 2255 | Seq. ID No. 2256 | Signal peptide | IM | 2 |
| 1135a | Seq. ID No. 2261 | Seq. ID No. 2262 | Non-secretory protein | IM | 1 |
| 1135b | Seq. ID No. 2263 | Seq. ID No. 2264 | Non-secretory protein | IM | 1 |
| 1140a | Seq. ID No. 2271 | Seq. ID No. 2272 | Signal peptide | IM | 2 |
| 1141b | Seq. ID No. 2279 | Seq. ID No. 2280 | Non-secretory protein | Cyto | 1 |
| 1144a | Seq. ID No. 2283 | Seq. ID No. 2284 | Signal peptide | IM | 2 |
| 1144b | Seq. ID No. 2285 | Seq. ID No. 2286 | Non-secretory protein | IM | 4 |
| 1146a | Seq. ID No. 2289 | Seq. ID No. 2290 | Signal peptide | IM | 3 |
| 1146d | Seq. ID No. 2295 | Seq. ID No. 2296 | Signal peptide | IM | 8 |
| 1152b | Seq. ID No. 2305 | Seq. ID No. 2306 | Signal peptide | IM | 5 |
| 1152c | Seq. ID No. 2307 | Seq. ID No. 2308 | Signal peptide | IM | 6 |
| 1155a | Seq. ID No. 2317 | Seq. ID No. 2318 | Signal peptide | IM | 3 |
| 1155b | Seq. ID No. 2319 | Seq. ID No. 2320 | Signal peptide | IM | 4 |
| 1155d | Seq. ID No. 2323 | Seq. ID No. 2324 | Signal peptide | IM | 6 |
| 1155e | Seq. ID No. 2325 | Seq. ID No. 2326 | Signal peptide | IM | 9 |
| 1165 | Seq. ID No. 2343 | Seq. ID No. 2344 | Signal peptide | OM | 1 |
| 1167b | Seq. ID No. 2349 | Seq. ID No. 2350 | Signal peptide | IM | 2 |
| 1180b | Seq. ID No. 2385 | Seq. ID No. 2386 | Non-secretory protein | IM | 2 |
| 1182a | Seq. ID No. 2393 | Seq. ID No. 2394 | Signal peptide | IM | 1 |
| 1194 | Seq. ID No. 2423 | Seq. ID No. 2424 | Non-secretory protein | IM | 1 |
| 1209 | Seq. ID No. 2453 | Seq. ID No. 2454 | Non-secretory protein | IM | 4 |
| 1224c | Seq. ID No. 2491 | Seq. ID No. 2492 | Signal peptide | IM | 6 |
| 1228a | Seq. ID No. 2497 | Seq. ID No. 2498 | Signal peptide | IM | 1 |
| 1228b | Seq. ID No. 2499 | Seq. ID No. 2500 | Signal peptide | IM | 2 |
| 1231 | Seq. ID No. 2507 | Seq. ID No. 2508 | Signal peptide | OM | 1 |
| 1234a | Seq. ID No. 2515 | Seq. ID No. 2516 | Signal peptide | IM | 4 |
| 1234b | Seq. ID No. 2517 | Seq. ID No. 2518 | Signal peptide | IM | 4 |
| 1235 | Seq. ID No. 2521 | Seq. ID No. 2522 | Non-secretory protein | Cyto | 1 |
| 1236a | Seq. ID No. 2523 | Seq. ID No. 2524 | Signal peptide | IM | 2 |
| 1239 | Seq. ID No. 2527 | Seq. ID No. 2528 | Signal peptide | OM | 1 |
| 1241b | Seq. ID No. 2533 | Seq. ID No. 2534 | Signal peptide | IM | 7 |
| 1241c | Seq. ID No. 2535 | Seq. ID No. 2536 | Signal peptide | IM | 7 |
| 1245a | Seq. ID No. 2541 | Seq. ID No. 2542 | Signal peptide | IM | 6 |
| 1245b | Seq. ID No. 2543 | Seq. ID No. 2544 | Signal peptide | IM | 10 |
| 1245d | Seq. ID No. 2547 | Seq. ID No. 2548 | Non-secretory protein | IM | 12 |
| 1245e | Seq. ID No. 2549 | Seq. ID No. 2550 | Non-secretory protein | IM | 12 |
| 1250b | Seq. ID No. 2557 | Seq. ID No. 2558 | Signal peptide | IM | 8 |
| 1253c | Seq. ID No. 2573 | Seq. ID No. 2574 | Non-secretory protein | IM | 7 |
| 1258a | Seq. ID No. 2583 | Seq. ID No. 2584 | Signal peptide | IM | 5 |
| 1267a | Seq. ID No. 2601 | Seq. ID No. 2602 | Signal peptide | IM | 2 |
| 1267b | Seq. ID No. 2603 | Seq. ID No. 2604 | Non-secretory protein | IM | 3 |
| 1275a | Seq. ID No. 2621 | Seq. ID No. 2622 | Signal peptide | Cyto | 1 |
| 1289 | Seq. ID No. 2653 | Seq. ID No. 2654 | Non-secretory protein | IM | 1 |
| 1308b | Seq. ID No. 2681 | Seq. ID No. 2682 | Signal peptide | IM | 4 |
| 1314b | Seq. ID No. 2691 | Seq. ID No. 2692 | Signal peptide | IM | 1 |
| 1319a | Seq. ID No. 2701 | Seq. ID No. 2702 | Signal peptide | IM | 4 |
| 1335a | Seq. ID No. 2733 | Seq. ID No. 2734 | Signal peptide | OM | 2 |
| 1338 | Seq. ID No. 2739 | Seq. ID No. 2740 | Non-secretory protein | IM | 1 |
| 1375 | Seq. ID No. 2801 | Seq. ID No. 2802 | Signal peptide | Cyto | 0 |
| 1379 | Seq. ID No. 2807 | Seq. ID No. 2808 | Non-secretory protein | IM | 1 |
| 1382 | Seq. ID No. 2815 | Seq. ID No. 2816 | Signal peptide | OM | 1 |
| 1384a | Seq. ID No. 2819 | Seq. ID No. 2820 | Signal peptide | IM | 2 |
| 1384b | Seq. ID No. 2821 | Seq. ID No. 2822 | Signal peptide | IM | 2 |
| 1384c | Seq. ID No. 2823 | Seq. ID No. 2824 | Signal peptide | IM | 2 |
| 1389a | Seq. ID No. 2833 | Seq. ID No. 2834 | Signal peptide | Cyto | 0 |
| 1393b | Seq. ID No. 2837 | Seq. ID No. 2838 | Signal peptide | IM | 2 |
| 1393c | Seq. ID No. 2839 | Seq. ID No. 2840 | Signal peptide | IM | 3 |
| 1395a | Seq. ID No. 2845 | Seq. ID No. 2846 | Signal peptide | Cyto | |
| 1399a | Seq. ID No. 2853 | Seq. ID No. 2854 | Signal peptide | IM | 2 |
| 1402c | Seq. ID No. 2861 | Seq. ID No. 2862 | Signal peptide | IM | 2 |
| 1407b | Seq. ID No. 2865 | Seq. ID No. 2866 | Non-secretory protein | IM | 1 |
| 1411a | Seq. ID No. 2873 | Seq. ID No. 2874 | Signal peptide | IM | 3 |
| 1411b | Seq. ID No. 2875 | Seq. ID No. 2876 | Non-secretory protein | IM | 4 |
| 1411c | Seq. ID No. 2877 | Seq. ID No. 2878 | Non-secretory protein | IM | 6 |
| 1414a | Seq. ID No. 2883 | Seq. ID No. 2884 | Non-secretory protein | IM | 0 |
| 1419 | Seq. ID No. 2895 | Seq. ID No. 2896 | Non-secretory protein | IM | |
| 1426a | Seq. ID No. 2905 | Seq. ID No. 2906 | Signal peptide | IM | 4 |
| 1439b | Seq. ID No. 2939 | Seq. ID No. 2940 | Signal peptide | IM | 5 |
| 1439c | Seq. ID No. 2941 | Seq. ID No. 2942 | Signal peptide | IM | 6 |
| 1440a | Seq. ID No. 2947 | Seq. ID No. 2948 | Signal peptide | IM | 6 |
| 1452a | Seq. ID No. 2981 | Seq. ID No. 2982 | Signal peptide | IM | 4 |
| 1452b | Seq. ID No. 2983 | Seq. ID No. 2984 | Signal peptide | IM | 4 |
| 1454 | Seq. ID No. 2987 | Seq. ID No. 2988 | Signal peptide | Cyto | 1 |
| 1455b | Seq. ID No. 2991 | Seq. ID No. 2992 | Signal peptide | OM | 1 |
| 1457a | Seq. ID No. 2995 | Seq. ID No. 2996 | Signal peptide | IM | 3 |
| 1459 | Seq. ID No. 3001 | Seq. ID No. 3002 | Non-secretory protein | IM | 1 |
| 1462b | Seq. ID No. 3005 | Seq. ID No. 3006 | Signal peptide | IM | 6 |
| 1467 | Seq. ID No. 3021 | Seq. ID No. 3022 | Non-secretory protein | Cyto | 2 |
| 1469b | Seq. ID No. 3025 | Seq. ID No. 3026 | Signal peptide | IM | 5 |
| 1475 | Seq. ID No. 3043 | Seq. ID No. 3044 | Signal peptide | OM | 1 |
| 1476 | Seq. ID No. 3045 | Seq. ID No. 3046 | Non-secretory protein | IM | 5 |
| 1488a | Seq. ID No. 3073 | Seq. ID No. 3074 | Signal peptide | IM | 1 |
| 1488b | Seq. ID No. 3075 | Seq. ID No. 3076 | Signal peptide | IM | 2 |
| 1520 | Seq. ID No. 3155 | Seq. ID No. 3156 | Signal peptide | Peri | 1 |
| 1530a | Seq. ID No. 3163 | Seq. ID No. 3164 | Signal peptide | OM | 1 |
| 1530b | Seq. ID No. 3165 | Seq. ID No. 3166 | Signal peptide | IM | 1 |
| 1533a | Seq. ID No. 3167 | Seq. ID No. 3168 | Non-secretory protein | IM | 0 |
| 1543 | Seq. ID No. 3183 | Seq. ID No. 3184 | Signal peptide | OM | 1 |
| 1557a | Seq. ID No. 3213 | Seq. ID No. 3214 | Signal peptide | IM | 4 |
| 1557b | Seq. ID No. 3215 | Seq. ID No. 3216 | Signal peptide | IM | 5 |
| 1569 | Seq. ID No. 3235 | Seq. ID No. 3236 | Non-secretory protein | IM | 2 |
| 1572c | Seq. ID No. 3245 | Seq. ID No. 3246 | Signal peptide | IM | 10 |
| 1580a | Seq. ID No. 3261 | Seq. ID No. 3262 | Signal peptide | IM | 1 |
| 1580b | Seq. ID No. 3263 | Seq. ID No. 3264 | Signal peptide | IM | 1 |
| 1581 | Seq. ID No. 3265 | Seq. ID No. 3266 | Non-secretory protein | IM | 1 |
| 1582b | Seq. ID No. 3269 | Seq. ID No. 3270 | Signal peptide | IM | 1 |
| 1582c | Seq. ID No. 3271 | Seq. ID No. 3272 | Non-secretory protein | IM | 1 |
| 1585a | Seq. ID No. 3275 | Seq. ID No. 3276 | Signal peptide | IM | 1 |
| 1585b | Seq. ID No. 3277 | Seq. ID No. 3278 | Signal peptide | IM | 1 |
| 1606a | Seq. ID No. 3317 | Seq. ID No. 3318 | Signal peptide | IM | 1 |
| 1608 | Seq. ID No. 3325 | Seq. ID No. 3326 | Signal peptide | Cyto | 1 |
| 1619 | Seq. ID No. 3337 | Seq. ID No. 3338 | Signal peptide | OM | 1 |
| 1625a | Seq. ID No. 3351 | Seq. ID No. 3352 | Signal peptide | IM | 4 |
| 1646a | Seq. ID No. 3389 | Seq. ID No. 3390 | Signal peptide | IM | 2 |
| 1649a | Seq. ID No. 3405 | Seq. ID No. 3406 | Signal peptide | Peri | 2 |
| 1657 | Seq. ID No. 3419 | Seq. ID No. 3420 | Non-secretory protein | Cyto | |
| 1659a | Seq. ID No. 3421 | Seq. ID No. 3422 | Signal peptide | OM | 1 |
| 1659b | Seq. ID No. 3423 | Seq. ID No. 3424 | Non-secretory protein | IM | 9 |
| 1673b | Seq. ID No. 3451 | Seq. ID No. 3452 | Signal peptide | IM | 8 |
| 1673c | Seq. ID No. 3453 | Seq. ID No. 3454 | Signal peptide | IM | 10 |
| 1673d | Seq. ID No. 3455 | Seq. ID No. 3456 | Signal peptide | IM | 12 |
| 1688a | Seq. ID No. 3491 | Seq. ID No. 3492 | Signal peptide | IM | 3 |
| 1688c | Seq. ID No. 3495 | Seq. ID No. 3496 | Non-secretory protein | IM | 11 |
| 1690a | Seq. ID No. 3503 | Seq. ID No. 3504 | Signal peptide | IM | 1 |
| 1690b | Seq. ID No. 3505 | Seq. ID No. 3506 | Signal peptide | IM | 2 |
| 1694c | Seq. ID No. 3517 | Seq. ID No. 3518 | Signal peptide | IM | 1 |
| 1694d | Seq. ID No. 3519 | Seq. ID No. 3520 | Signal peptide | IM | 1 |
| 1705a | Seq. ID No. 3545 | Seq. ID No. 3546 | Non-secretory protein | Cyto | |
| 1705b | Seq. ID No. 3547 | Seq. ID No. 3548 | Non-secretory protein | Cyto | |
| 1718a | Seq. ID No. 3571 | Seq. ID No. 3572 | Non-secretory protein | Cyto | |
| 1735a | Seq. ID No. 3593 | Seq. ID No. 3594 | Signal peptide | OM | 1 |
| 1745 | Seq. ID No. 3617 | Seq. ID No. 3618 | Signal peptide | Peri | 0 |
| 1749a | Seq. ID No. 3621 | Seq. ID No. 3622 | Signal peptide | IM | 3 |
| 1752b | Seq. ID No. 3629 | Seq. ID No. 3630 | Signal peptide | Peri | 1 |
| 1769b | Seq. ID No. 3663 | Seq. ID No. 3664 | Non-secretory protein | IM | 7 |
| 1790a | Seq. ID No. 3701 | Seq. ID No. 3702 | Non-secretory protein | Cyto | 2 |
| 1790b | Seq. ID No. 3703 | Seq. ID No. 3704 | Non-secretory protein | Cyto | 3 |
| 1798a | Seq. ID No. 3723 | Seq. ID No. 3724 | Signal peptide | IM | 2 |
| 1804a | Seq. ID No. 3737 | Seq. ID No. 3738 | Non-secretory protein | IM | 2 |
| 1805 | Seq. ID No. 3741 | Seq. ID No. 3742 | Signal peptide | IM | 3 |
| 1806b | Seq. ID No. 3745 | Seq. ID No. 3746 | Signal peptide | IM | 5 |
| 1806c | Seq. ID No. 3747 | Seq. ID No. 3748 | Non-secretory protein | IM | 5 |
| 1808d | Seq. ID No. 3759 | Seq. ID No. 3760 | Non-secretory protein | IM | 15 |
| 1809b | Seq. ID No. 3763 | Seq. ID No. 3764 | Signal peptide | IM | 7 |
| 1809d | Seq. ID No. 3767 | Seq. ID No. 3768 | Non-secretory protein | IM | 12 |
| 1809e | Seq. ID No. 3769 | Seq. ID No. 3770 | Non-secretory protein | IM | 14 |
| 1809f | Seq. ID No. 3771 | Seq. ID No. 3772 | Non-secretory protein | IM | 14 |
| 1813 | Seq. ID No. 3785 | Seq. ID No. 3786 | Non-secretory protein | IM | 1 |
| 1816a | Seq. ID No. 3793 | Seq. ID No. 3794 | Signal peptide | Peri | 1 |
| 1825 | Seq. ID No. 3813 | Seq. ID No. 3814 | Signal peptide | IM | 2 |
| 1838a | Seq. ID No. 3837 | Seq. ID No. 3838 | Signal peptide | IM | 3 |
| 1838b | Seq. ID No. 3839 | Seq. ID No. 3840 | Signal peptide | IM | 4 |
| 1845b | Seq. ID No. 3861 | Seq. ID No. 3862 | Signal peptide | IM | 8 |
| 1848a | Seq. ID No. 3865 | Seq. ID No. 3866 | Signal peptide | IM | 7 |
| 1857b | Seq. ID No. 3889 | Seq. ID No. 3890 | Signal peptide | IM | 5 |
| 1857c | Seq. ID No. 3891 | Seq. ID No. 3892 | Signal peptide | IM | 6 |
| 1858 | Seq. ID No. 3895 | Seq. ID No. 3896 | Signal peptide | Cyto | 0 |
| 1868 | Seq. ID No. 3915 | Seq. ID No. 3916 | Signal peptide | IM | 1 |
| 1873b | Seq. ID No. 3923 | Seq. ID No. 3924 | Signal peptide | Peri | 1 |
| 1873c | Seq. ID No. 3925 | Seq. ID No. 3926 | Signal peptide | Peri | 1 |
| 1875c | Seq. ID No. 3929 | Seq. ID No. 3930 | Non-secretory protein | IM | 1 |
| 1896b | Seq. ID No. 3965 | Seq. ID No. 3966 | Non-secretory protein | IM | 2 |
| 1898b | Seq. ID No. 3975 | Seq. ID No. 3976 | Signal peptide | IM | 4 |
| 1900 | Seq. ID No. 3979 | Seq. ID No. 3980 | Non-secretory protein | Cyto | 1 |
| 1917a | Seq. ID No. 3995 | Seq. ID No. 3996 | Signal peptide | IM | 0 |
| 1917b | Seq. ID No. 3997 | Seq. ID No. 3998 | Signal peptide | IM | 0 |
| 1919c | Seq. ID No. 4009 | Seq. ID No. 4010 | Signal peptide | IM | 1 |
| 1936 | Seq. ID No. 4039 | Seq. ID No. 4040 | Signal peptide | IM | 6 |
| 1937a | Seq. ID No. 4041 | Seq. ID No. 4042 | Signal peptide | IM | 2 |
| 1941b | Seq. ID No. 4061 | Seq. ID No. 4062 | Signal peptide | IM | 4 |
| 1950 | Seq. ID No. 4079 | Seq. ID No. 4080 | Signal peptide | OM | 1 |
| 1951a | Seq. ID No. 4081 | Seq. ID No. 4082 | Signal peptide | IM | 3 |
| 1960c | Seq. ID No. 4111 | Seq. ID No. 4112 | Signal peptide | IM | 1 |
| 1961 | Seq. ID No. 4113 | Seq. ID No. 4114 | Non-secretory protein | IM | 1 |
| 1972 | Seq. ID No. 4135 | Seq. ID No. 4136 | Signal peptide | Cyto | 1 |
| 1976 | Seq. ID No. 4139 | Seq. ID No. 4140 | Signal peptide | IM | 1 |
| 1981 | Seq. ID No. 4155 | Seq. ID No. 4156 | Non-secretory protein | Cyto | 1 |
| 1991 | Seq. ID No. 4181 | Seq. ID No. 4182 | Signal peptide | Cyto | 1 |
| 2008 | Seq. ID No. 4203 | Seq. ID No. 4204 | Non-secretory protein | IM | 2 |
| 2016a | Seq. ID No. 4221 | Seq. ID No. 4222 | Signal peptide | IM | 2 |
| 2024b | Seq. ID No. 4247 | Seq. ID No. 4248 | Signal peptide | IM | 2 |
| 2025 | Seq. ID No. 4253 | Seq. ID No. 4254 | Signal peptide | OM | 1 |
| 2030a | Seq. ID No. 4257 | Seq. ID No. 4258 | Signal peptide | IM | 4 |
| 2030b | Seq. ID No. 4259 | Seq. ID No. 4260 | Signal peptide | IM | 5 |
| 2030c | Seq. ID No. 4261 | Seq. ID No. 4262 | Signal peptide | IM | 5 |
| 2030d | Seq. ID No. 4263 | Seq. ID No. 4264 | Signal peptide | IM | 5 |
| 2035b | Seq. ID No. 4269 | Seq. ID No. 4270 | Signal peptide | Cyto | 0 |
| 2059a | Seq. ID No. 4311 | Seq. ID No. 4312 | Signal peptide | IM | 2 |
| 2092 | Seq. ID No. 4379 | Seq. ID No. 4380 | Signal peptide | IM | 2 |
| 2096c | Seq. ID No. 4389 | Seq. ID No. 4390 | Signal peptide | IM | 10 |
| 2096d | Seq. ID No. 4391 | Seq. ID No. 4392 | Signal peptide | IM | 10 |
| 2140b | Seq. ID No. 4471 | Seq. ID No. 4472 | Non-secretory protein | IM | 5 |
| 2142a | Seq. ID No. 4473 | Seq. ID No. 4474 | Signal peptide | IM | 1 |
| 2146 | Seq. ID No. 4481 | Seq. ID No. 4482 | Signal peptide | OM | 1 |
| 2161a | Seq. ID No. 4525 | Seq. ID No. 4526 | Non-secretory protein | Cyto | 1 |
| 2175 | Seq. ID No. 4551 | Seq. ID No. 4552 | Non-secretory protein | IM | 2 |
| 2177b | Seq. ID No. 4561 | Seq. ID No. 4562 | Non-secretory protein | IM | 1 |
| 2182 | Seq. ID No. 4573 | Seq. ID No. 4574 | Non-secretory protein | IM | 1 |
| 2208a | Seq. ID No. 4617 | Seq. ID No. 4618 | Non-secretory protein | Cyto | 0 |
| 2219a | Seq. ID No. 4639 | Seq. ID No. 4640 | Signal peptide | IM | 1 |
| 2225 | Seq. ID No. 4659 | Seq. ID No. 4660 | Signal peptide | OM | 0 |
| 2236a | Seq. ID No. 4681 | Seq. ID No. 4682 | Signal peptide | IM | 2 |
| 2246 | Seq. ID No. 4701 | Seq. ID No. 4702 | Non-secretory protein | IM | |
| 2249 | Seq. ID No. 4705 | Seq. ID No. 4706 | Signal peptide | OM | 1 |
| 2253d | Seq. ID No. 4713 | Seq. ID No. 4714 | Non-secretory protein | IM | 7 |
| 2255b | Seq. ID No. 4717 | Seq. ID No. 4718 | Signal peptide | IM | 6 |
| 2257 | Seq. ID No. 4721 | Seq. ID No. 4722 | Non-secretory protein | IM | 0 |
| 2264b | Seq. ID No. 4737 | Seq. ID No. 4738 | Signal peptide | IM | 1 |
| 2268 | Seq. ID No. 4741 | Seq. ID No. 4742 | Non-secretory protein | Cyto | 0 |
| 2300 | Seq. ID No. 4813 | Seq. ID No. 4814 | Signal peptide | IM | 4 |
| 2302 | Seq. ID No. 4817 | Seq. ID No. 4818 | Non-secretory protein | IM | 1 |
| 2303 | Seq. ID No. 4819 | Seq. ID No. 4820 | Non-secretory protein | IM | 1 |
| 2309a | Seq. ID No. 4829 | Seq. ID No. 4830 | Non-secretory protein | IM | 2 |
| 2309b | Seq. ID No. 4831 | Seq. ID No. 4832 | Non-secretory protein | IM | 2 |
| 2317 | Seq. ID No. 4851 | Seq. ID No. 4852 | Non-secretory protein | IM | 2 |
| 2329a | Seq. ID No. 4869 | Seq. ID No. 4870 | Signal peptide | IM | 1 |
| 2329d | Seq. ID No. 4875 | Seq. ID No. 4876 | Signal peptide | IM | 7 |
| 2350a | Seq. ID No. 4913 | Seq. ID No. 4914 | Non-secretory protein | Cyto | |
| 2357 | Seq. ID No. 4919 | Seq. ID No. 4920 | Signal peptide | IM | 2 |
| 2363a | Seq. ID No. 4929 | Seq. ID No. 4930 | Signal peptide | IM | 1 |
| 2363b | Seq. ID No. 4931 | Seq. ID No. 4932 | Signal peptide | IM | 1 |
| 2386c | Seq. ID No. 4983 | Seq. ID No. 4984 | Non-secretory protein | IM | 0 |
| 2394 | Seq. ID No. 4993 | Seq. ID No. 4994 | Non-secretory protein | IM | 1 |
| 2402a | Seq. ID No. 5011 | Seq. ID No. 5012 | Non-secretory protein | IM | 1 |
| 2407a | Seq. ID No. 5023 | Seq. ID No. 5024 | Non-secretory protein | IM | 0 |
| 2412 | Seq. ID No. 5031 | Seq. ID No. 5032 | Signal peptide | IM | 2 |
| 2413a | Seq. ID No. 5033 | Seq. ID No. 5034 | Non-secretory protein | Cyto | 0 |
| 2413b | Seq. ID No. 5035 | Seq. ID No. 5036 | Non-secretory protein | Peri | 0 |
| 2416c | Seq. ID No. 5043 | Seq. ID No. 5044 | Non-secretory protein | IM | 1 |
| 2423a | Seq. ID No. 5061 | Seq. ID No. 5062 | Signal peptide | IM | 5 |
| 2423b | Seq. ID No. 5063 | Seq. ID No. 5064 | Signal peptide | IM | 5 |
| 2424c | Seq. ID No. 5069 | Seq. ID No. 5070 | Signal peptide | IM | 1 |
| 2445 | Seq. ID No. 5103 | Seq. ID No. 5104 | Signal peptide | IM | 1 |
| 2456b | Seq. ID No. 5121 | Seq. ID No. 5122 | Non-secretory protein | IM | 5 |
| 2456c | Seq. ID No. 5123 | Seq. ID No. 5124 | Signal peptide | IM | 8 |
| 2458b | Seq. ID No. 5131 | Seq. ID No. 5132 | Signal peptide | IM | 6 |
| 2458c | Seq. ID No. 5133 | Seq. ID No. 5134 | Signal peptide | IM | 7 |
| 2458d | Seq. ID No. 5135 | Seq. ID No. 5136 | Signal peptide | IM | 7 |
| 2458e | Seq. ID No. 5137 | Seq. ID No. 5138 | Signal peptide | IM | 8 |
| 2458f | Seq. ID No. 5139 | Seq. ID No. 5140 | Signal peptide | IM | 9 |
| 2458g | Seq. ID No. 5141 | Seq. ID No. 5142 | Signal peptide | IM | 9 |
| 2463a | Seq. ID No. 5151 | Seq. ID No. 5152 | Non-secretory protein | IM | 1 |
| 2468a | Seq. ID No. 5159 | Seq. ID No. 5160 | Signal peptide | IM | 1 |
| 2468b | Seq. ID No. 5161 | Seq. ID No. 5162 | Non-secretory protein | IM | 1 |
| 2469b | Seq. ID No. 5165 | Seq. ID No. 5166 | Signal peptide | IM | 2 |
| 2470 | Seq. ID No. 5167 | Seq. ID No. 5168 | Non-secretory protein | Cyto | 0 |
| 2474b | Seq. ID No. 5175 | Seq. ID No. 5176 | Signal peptide | IM | 3 |
| 2474c | Seq. ID No. 5177 | Seq. ID No. 5178 | Non-secretory protein | IM | 3 |
| 2490 | Seq. ID No. 5205 | Seq. ID No. 5206 | Signal peptide | IM | 1 |
| 2492a | Seq. ID No. 5207 | Seq. ID No. 5208 | Signal peptide | IM | 1 |
| 2494 | Seq. ID No. 5211 | Seq. ID No. 5212 | Non-secretory protein | IM | 0 |
| 2495b | Seq. ID No. 5215 | Seq. ID No. 5216 | Non-secretory protein | IM | 10 |
| 2497b | Seq. ID No. 5219 | Seq. ID No. 5220 | Signal peptide | Peri | 2 |
| 2500a | Seq. ID No. 5223 | Seq. ID No. 5224 | Signal peptide | OM | 1 |
| 2525a | Seq. ID No. 5285 | Seq. ID No. 5286 | Signal peptide | IM | 1 |
| 2525b | Seq. ID No. 5287 | Seq. ID No. 5288 | Non-secretory protein | IM | 1 |
| 2547b | Seq. ID No. 5329 | Seq. ID No. 5330 | Signal peptide | IM | 1 |
| 2547c | Seq. ID No. 5331 | Seq. ID No. 5332 | Signal peptide | IM | 1 |
| 2564b | Seq. ID No. 5377 | Seq. ID No. 5378 | Signal peptide | OM | 1 |
| 2564c | Seq. ID No. 5379 | Seq. ID No. 5380 | Signal peptide | OM | 1 |
| 2564d | Seq. ID No. 5381 | Seq. ID No. 5382 | Non-secretory protein | IM | 1 |
| 2567a | Seq. ID No. 5385 | Seq. ID No. 5386 | Signal peptide | IM | 4 |
| 2582a | Seq. ID No. 5405 | Seq. ID No. 5406 | Signal peptide | IM | 4 |
| 2582b | Seq. ID No. 5407 | Seq. ID No. 5408 | Non-secretory protein | IM | 4 |
| 2610 | Seq. ID No. 5453 | Seq. ID No. 5454 | Signal peptide | IM | 1 |
| 2616a | Seq. ID No. 5459 | Seq. ID No. 5460 | Signal peptide | IM | 2 |
| 2616b | Seq. ID No. 5461 | Seq. ID No. 5462 | Signal peptide | IM | 3 |
| 2616c | Seq. ID No. 5463 | Seq. ID No. 5464 | Signal peptide | IM | 4 |
| 2618b | Seq. ID No. 5481 | Seq. ID No. 5482 | Non-secretory protein | IM | |
| 2628 | Seq. ID No. 5509 | Seq. ID No. 5510 | Signal peptide | IM | 1 |
| 2637a | Seq. ID No. 5529 | Seq. ID No. 5530 | Signal peptide | OM | 1 |
| 2637b | Seq. ID No. 5531 | Seq. ID No. 5532 | Signal peptide | IM | 1 |
| 2655b | Seq. ID No. 5557 | Seq. ID No. 5558 | Non-secretory protein | IM | 1 |
| 2660 | Seq. ID No. 5569 | Seq. ID No. 5570 | Signal peptide | Peri | 1 |
| 2665 | Seq. ID No. 5577 | Seq. ID No. 5578 | Non-secretory protein | IM | 1 |
| 2683a | Seq. ID No. 5623 | Seq. ID No. 5624 | Signal peptide | IM | 1 |
| 2683b | Seq. ID No. 5625 | Seq. ID No. 5626 | Signal peptide | IM | 1 |
| 2690a | Seq. ID No. 5643 | Seq. ID No. 5644 | Signal peptide | Peri | 1 |
| 2696a | Seq. ID No. 5659 | Seq. ID No. 5660 | Signal peptide | IM | 8 |
| 2696b | Seq. ID No. 5661 | Seq. ID No. 5662 | Non-secretory protein | IM | 8 |
| 2718b | Seq. ID No. 5683 | Seq. ID No. 5684 | Signal peptide | IM | 3 |
| 2718c | Seq. ID No. 5685 | Seq. ID No. 5686 | Signal peptide | IM | 4 |
| 2728a | Seq. ID No. 5709 | Seq. ID No. 5710 | Signal peptide | IM | 1 |
| 2728b | Seq. ID No. 5711 | Seq. ID No. 5712 | Signal peptide | IM | 1 |
| 2738c | Seq. ID No. 5733 | Seq. ID No. 5734 | Signal peptide | IM | 12 |
| 2742b | Seq. ID No. 5739 | Seq. ID No. 5740 | Signal peptide | Peri | |
| 2749b | Seq. ID No. 5765 | Seq. ID No. 5766 | Signal peptide | IM | 4 |
| 2760a | Seq. ID No. 5797 | Seq. ID No. 5798 | Signal peptide | Peri | 1 |
| 2769a | Seq. ID No. 5827 | Seq. ID No. 5828 | Non-secretory protein | IM | 0 |
| 2799a | Seq. ID No. 5889 | Seq. ID No. 5890 | Signal peptide | OM | 1 |
| 2801a | Seq. ID No. 5893 | Seq. ID No. 5894 | Signal peptide | IM | 1 |
| 2810a | Seq. ID No. 5905 | Seq. ID No. 5906 | Signal peptide | IM | 3 |
| 2810c | Seq. ID No. 5909 | Seq. ID No. 5910 | Signal peptide | IM | 4 |
| 2810d | Seq. ID No. 5911 | Seq. ID No. 5912 | Non-secretory protein | IM | 4 |
| 2818b | Seq. ID No. 5923 | Seq. ID No. 5924 | Signal peptide | IM | 5 |
| 2837a | Seq. ID No. 5963 | Seq. ID No. 5964 | Non-secretory protein | IM | 2 |
| 2837b | Seq. ID No. 5965 | Seq. ID No. 5966 | Non-secretory protein | IM | 3 |
| 2869b | Seq. ID No. 6025 | Seq. ID No. 6026 | Signal peptide | OM | 1 |
| 2869c | Seq. ID No. 6027 | Seq. ID No. 6028 | Signal peptide | IM | 1 |
| 2872 | Seq. ID No. 6031 | Seq. ID No. 6032 | Non-secretory protein | Cyto | |
| 2881 | Seq. ID No. 6043 | Seq. ID No. 6044 | Signal peptide | IM | 1 |
| 2882 | Seq. ID No. 6045 | Seq. ID No. 6046 | Non-secretory protein | IM | 2 |
| 2883a | Seq. ID No. 6047 | Seq. ID No. 6048 | Signal peptide | IM | 6 |
| 2883d | Seq. ID No. 6053 | Seq. ID No. 6054 | Signal peptide | IM | 8 |
| 2887a | Seq. ID No. 6059 | Seq. ID No. 6060 | Signal peptide | Peri | 1 |
| 2909c | Seq. ID No. 6109 | Seq. ID No. 6110 | Signal peptide | IM | 4 |
| 2909d | Seq. ID No. 6111 | Seq. ID No. 6112 | Signal peptide | IM | 4 |
| 2913a | Seq. ID No. 6115 | Seq. ID No. 6116 | Signal peptide | IM | 2 |
| 2913b | Seq. ID No. 6117 | Seq. ID No. 6118 | Signal peptide | IM | 2 |
| 2917 | Seq. ID No. 6127 | Seq. ID No. 6128 | Non-secretory protein | OM | 1 |
| 2920 | Seq. ID No. 6131 | Seq. ID No. 6132 | Non-secretory protein | IM | 2 |
| 2922b | Seq. ID No. 6135 | Seq. ID No. 6136 | Signal peptide | Cyto | 0 |
| 2923a | Seq. ID No. 6137 | Seq. ID No. 6138 | Signal peptide | Cyto | |
| 2931a | Seq. ID No. 6161 | Seq. ID No. 6162 | Signal peptide | IM | 5 |
| 2931c | Seq. ID No. 6165 | Seq. ID No. 6166 | Signal peptide | IM | 13 |
| 2941a | Seq. ID No. 6183 | Seq. ID No. 6184 | Signal peptide | IM | 2 |
| 2943a | Seq. ID No. 6189 | Seq. ID No. 6190 | Signal peptide | IM | 3 |
| 2943b | Seq. ID No. 6191 | Seq. ID No. 6192 | Signal peptide | IM | 5 |
| 2943c | Seq. ID No. 6193 | Seq. ID No. 6194 | Non-secretory protein | IM | 6 |
| 2948c | Seq. ID No. 6207 | Seq. ID No. 6208 | Non-secretory protein | IM | 6 |
| 2948d | Seq. ID No. 6209 | Seq. ID No. 6210 | Signal peptide | IM | 7 |
| 2952 | Seq. ID No. 6217 | Seq. ID No. 6218 | Signal peptide | Cyto | 1 |
| 2955c | Seq. ID No. 6225 | Seq. ID No. 6226 | Signal peptide | IM | 6 |
| 2957 | Seq. ID No. 6235 | Seq. ID No. 6236 | Signal peptide | IM | 4 |
| 2959 | Seq. ID No. 6243 | Seq. ID No. 6244 | Signal peptide | IM | 2 |
| 2962b | Seq. ID No. 6249 | Seq. ID No. 6250 | Signal peptide | IM | 1 |
| 2965a | Seq. ID No. 6253 | Seq. ID No. 6254 | Signal peptide | Cyto | 1 |
| 2966 | Seq. ID No. 6259 | Seq. ID No. 6260 | Signal peptide | IM | 1 |
| 2970a | Seq. ID No. 6265 | Seq. ID No. 6266 | Signal peptide | IM | 1 |
| 2970b | Seq. ID No. 6267 | Seq. ID No. 6268 | Signal peptide | IM | 1 |
| 2974b | Seq. ID No. 6275 | Seq. ID No. 6276 | Signal peptide | IM | 4 |
| 2978a | Seq. ID No. 6283 | Seq. ID No. 6284 | Signal peptide | Peri | 1 |
| 2978b | Seq. ID No. 6285 | Seq. ID No. 6286 | Signal peptide | Peri | 1 |
| 2986b | Seq. ID No. 6303 | Seq. ID No. 6304 | Signal peptide | IM | 7 |
| 2993a | Seq. ID No. 6325 | Seq. ID No. 6326 | Signal peptide | OM | 0 |
| 2993b | Seq. ID No. 6327 | Seq. ID No. 6328 | Signal peptide | OM | 0 |
| 3016a | Seq. ID No. 6363 | Seq. ID No. 6364 | Signal peptide | IM | 4 |
| 3016b | Seq. ID No. 6365 | Seq. ID No. 6366 | Signal peptide | IM | 7 |
| 3016c | Seq. ID No. 6367 | Seq. ID No. 6368 | Non-secretory protein | IM | 7 |
| 3024b | Seq. ID No. 6385 | Seq. ID No. 6386 | Non-secretory protein | Cyto | 1 |
| 3042a | Seq. ID No. 6425 | Seq. ID No. 6426 | Signal peptide | Cyto | 1 |
| 3042b | Seq. ID No. 6427 | Seq. ID No. 6428 | Signal peptide | Cyto | 1 |
| 3043a | Seq. ID No. 6429 | Seq. ID No. 6430 | Signal peptide | IM | 2 |
| 3043b | Seq. ID No. 6431 | Seq. ID No. 6432 | Signal peptide | IM | 2 |
| 3054 | Seq. ID No. 6453 | Seq. ID No. 6454 | Non-secretory protein | Cyto | |
| 3061 | Seq. ID No. 6465 | Seq. ID No. 6466 | Non-secretory protein | Peri | 1 |
| 3063a | Seq. ID No. 6469 | Seq. ID No. 6470 | Non-secretory protein | Cyto | 0 |
| 3063b | Seq. ID No. 6471 | Seq. ID No. 6472 | Non-secretory protein | Cyto | 0 |
| 3066 | Seq. ID No. 6479 | Seq. ID No. 6480 | Signal peptide | Cyto | 0 |
| 3095 | Seq. ID No. 6539 | Seq. ID No. 6540 | Non-secretory protein | IM | 1 |
| 3096a | Seq. ID No. 6541 | Seq. ID No. 6542 | Signal peptide | IM | 1 |
| 3107a | Seq. ID No. 6571 | Seq. ID No. 6572 | Signal peptide | OM | 1 |
| 3107b | Seq. ID No. 6573 | Seq. ID No. 6574 | Signal peptide | OM | 2 |
| 3145c | Seq. ID No. 6639 | Seq. ID No. 6640 | Signal peptide | OM | 1 |
| 3147b | Seq. ID No. 6643 | Seq. ID No. 6644 | Signal peptide | IM | 0 |
| 3147c | Seq. ID No. 6645 | Seq. ID No. 6646 | Signal peptide | OM | 0 |

Listed in Table 5 are 936 ORFs that meet the criteria of having a signal peptide based on the HMM SignalP program. Of these ORFs, the PSORT program predicted 71 ORFs to be localized to the periplasm (Peri); 93 ORFs to be localized to the outer membrane (OM); 663 ORFs to be localized to the cytoplasmic inner membrane (IM); and 109 ORFs to be localized to the cytoplasm. Shown for all ORFs is the predicted number of transmembrane domains predicted by the TopPred2 software (listed in the MSD column).

TABLE 5
SIGNALP: SIGNAL PEPTIDE

| ORF | DNA SEQ ID | Protein SEQ ID | | | |
|---|---|---|---|---|---|
| Number | Number | Number | SignalP | PSORT | MSD |
| 2a | Seq. ID No. 3 | Seq. ID No. 4 | NO | IM | 3 |
| 4a | Seq. ID No. 7 | Seq. ID No. 8 | Yes | IM | 4 |
| 4b | Seq. ID No. 9 | Seq. ID No. 10 | Maybe | IM | 10 |
| 11 | Seq. ID No. 19 | Seq. ID No. 20 | maybe | Cyto | 0 |
| 13a | Seq. ID No. 21 | Seq. ID No. 22 | NO | Cyto | 0 |
| 21b | Seq. ID No. 31 | Seq. ID No. 32 | maybe | Cyto | 1 |
| 23a | Seq. ID No. 33 | Seq. ID No. 34 | maybe | IM | 1 |
| 38a | Seq. ID No. 45 | Seq. ID No. 46 | yes | OM | 1 |
| 38b | Seq. ID No. 47 | Seq. ID No. 48 | yes | OM | 1 |
| 39a | Seq. ID No. 49 | Seq. ID No. 50 | maybe | IM | 4 |
| 40 | Seq. ID No. 53 | Seq. ID No. 54 | yes | IM | 1 |
| 44a | Seq. ID No. 65 | Seq. ID No. 66 | yes | IM | 2 |
| 44b | Seq. ID No. 67 | Seq. ID No. 68 | yes | IM | 2 |
| 51a | Seq. ID No. 85 | Seq. ID No. 86 | maybe | IM | 5 |
| 51b | Seq. ID No. 87 | Seq. ID No. 88 | yes | IM | 9 |
| 61 | Seq. ID No. 105 | Seq. ID No. 106 | NO | Cyto | 2 |
| 62c | Seq. ID No. 111 | Seq. ID No. 112 | NO | IM | 1 |
| 66a | Seq. ID No. 123 | Seq. ID No. 124 | NO | OM | 1 |
| 73a | Seq. ID No. 131 | Seq. ID No. 132 | YES | Cyto | 1 |
| 73b | Seq. ID No. 133 | Seq. ID No. 134 | YES | Cyto | 1 |
| 80a | Seq. ID No. 141 | Seq. ID No. 142 | maybe | IM | 4 |
| 86 | Seq. ID No. 159 | Seq. ID No. 160 | yes | Peri | 1 |
| 87a | Seq. ID No. 161 | Seq. ID No. 162 | YES | OM | 1 |
| 87b | Seq. ID No. 163 | Seq. ID No. 164 | YES | IM | 1 |
| 91a | Seq. ID No. 171 | Seq. ID No. 172 | maybe | OM | 0 |
| 99 | Seq. ID No. 191 | Seq. ID No. 192 | maybe | IM | 10 |
| 109a | Seq. ID No. 209 | Seq. ID No. 210 | maybe | IM | 5 |
| 109b | Seq. ID No. 211 | Seq. ID No. 212 | yes | IM | 7 |
| 109c | Seq. ID No. 213 | Seq. ID No. 214 | YES | IM | 8 |
| 109d | Seq. ID No. 215 | Seq. ID No. 216 | YES | IM | 8 |
| 111a | Seq. ID No. 217 | Seq. ID No. 218 | yes | IM | 6 |
| 114 | Seq. ID No. 225 | Seq. ID No. 226 | YES | OM | 1 |
| 124a | Seq. ID No. 235 | Seq. ID No. 236 | yes | IM | 1 |
| 124b | Seq. ID No. 237 | Seq. ID No. 238 | yes | IM | 3 |
| 129a | Seq. ID No. 249 | Seq. ID No. 250 | YES | IM | 6 |
| 129b | Seq. ID No. 251 | Seq. ID No. 252 | YES | IM | 9 |
| 132a | Seq. ID No. 255 | Seq. ID No. 256 | maybe | IM | 2 |
| 132b | Seq. ID No. 257 | Seq. ID No. 258 | maybe | IM | 2 |
| 132c | Seq. ID No. 259 | Seq. ID No. 260 | maybe | IM | 2 |
| 138a | Seq. ID No. 271 | Seq. ID No. 272 | yes | IM | 6 |
| 138b | Seq. ID No. 273 | Seq. ID No. 274 | maybe | IM | 6 |
| 143 | Seq. ID No. 277 | Seq. ID No. 278 | maybe | IM | 2 |
| 145a | Seq. ID No. 285 | Seq. ID No. 286 | yes | IM | 3 |
| 145b | Seq. ID No. 287 | Seq. ID No. 288 | YES | IM | 10 |
| 145c | Seq. ID No. 289 | Seq. ID No. 290 | YES | IM | 12 |
| 162a | Seq. ID No. 327 | Seq. ID No. 328 | yes | IM | 2 |
| 162b | Seq. ID No. 329 | Seq. ID No. 330 | maybe | IM | 3 |
| 173 | Seq. ID No. 357 | Seq. ID No. 358 | maybe | IM | 1 |
| 174 | Seq. ID No. 359 | Seq. ID No. 360 | maybe | Peri | 1 |
| 180a | Seq. ID No. 373 | Seq. ID No. 374 | maybe | IM | 2 |
| 180b | Seq. ID No. 375 | Seq. ID No. 376 | YES | IM | 10 |
| 191b | Seq. ID No. 393 | Seq. ID No. 394 | maybe | IM | 1 |
| 194a | Seq. ID No. 401 | Seq. ID No. 402 | maybe | Cyto | 1 |
| 194b | Seq. ID No. 403 | Seq. ID No. 404 | maybe | Cyto | 1 |
| 223a | Seq. ID No. 455 | Seq. ID No. 456 | yes | Cyto | 1 |
| 223b | Seq. ID No. 457 | Seq. ID No. 458 | yes | Cyto | 1 |
| 235a | Seq. ID No. 477 | Seq. ID No. 478 | maybe | Cyto | 0 |
| 239a | Seq. ID No. 487 | Seq. ID No. 488 | NO | Cyto | 2 |
| 239b | Seq. ID No. 489 | Seq. ID No. 490 | NO | Cyto | 2 |
| 239c | Seq. ID No. 491 | Seq. ID No. 492 | NO | Cyto | 2 |
| 240 | Seq. ID No. 493 | Seq. ID No. 494 | maybe | Cyto | |
| 241 | Seq. ID No. 495 | Seq. ID No. 496 | yes | IM | 1 |
| 245a | Seq. ID No. 497 | Seq. ID No. 498 | YES | IM | 5 |
| 245b | Seq. ID No. 499 | Seq. ID No. 500 | yes | IM | 10 |
| 246a | Seq. ID No. 503 | Seq. ID No. 504 | YES | IM | 7 |
| 246b | Seq. ID No. 505 | Seq. ID No. 506 | YES | IM | 9 |
| 248a | Seq. ID No. 509 | Seq. ID No. 510 | maybe | IM | 8 |
| 248b | Seq. ID No. 511 | Seq. ID No. 512 | yes | IM | 9 |
| 249a | Seq. ID No. 513 | Seq. ID No. 514 | yes | OM | 1 |
| 249b | Seq. ID No. 515 | Seq. ID No. 516 | yes | IM | 1 |
| 255a | Seq. ID No. 525 | Seq. ID No. 526 | yes | IM | 6 |
| 255b | Seq. ID No. 527 | Seq. ID No. 528 | YES | IM | 10 |
| 263a | Seq. ID No. 545 | Seq. ID No. 546 | yes | OM | 1 |
| 263b | Seq. ID No. 547 | Seq. ID No. 548 | yes | IM | 1 |
| 264b | Seq. ID No. 549 | Seq. ID No. 550 | YES | IM | 1 |
| 268 | Seq. ID No. 557 | Seq. ID No. 558 | yes | Cyto | 1 |
| 273a | Seq. ID No. 567 | Seq. ID No. 568 | maybe | IM | 8 |
| 273b | Seq. ID No. 569 | Seq. ID No. 570 | yes | IM | 12 |
| 277c | Seq. ID No. 575 | Seq. ID No. 576 | YES | OM | 1 |
| 279a | Seq. ID No. 577 | Seq. ID No. 578 | maybe | IM | 3 |
| 279b | Seq. ID No. 579 | Seq. ID No. 580 | maybe | IM | 5 |
| 283a | Seq. ID No. 591 | Seq. ID No. 592 | yes | OM | 1 |
| 283b | Seq. ID No. 593 | Seq. ID No. 594 | yes | OM | 1 |
| 285b | Seq. ID No. 599 | Seq. ID No. 600 | maybe | Cyto | |
| 297 | Seq. ID No. 617 | Seq. ID No. 618 | YES | Peri | 1 |
| 311 | Seq. ID No. 637 | Seq. ID No. 638 | yes | IM | 6 |
| 313a | Seq. ID No. 639 | Seq. ID No. 640 | yes | IM | 2 |
| 313b | Seq. ID No. 641 | Seq. ID No. 642 | yes | IM | 3 |
| 313c | Seq. ID No. 643 | Seq. ID No. 644 | maybe | IM | 6 |
| 313d | Seq. ID No. 645 | Seq. ID No. 646 | maybe | IM | 6 |
| 313e | Seq. ID No. 647 | Seq. ID No. 648 | maybe | IM | 6 |
| 317a | Seq. ID No. 653 | Seq. ID No. 654 | yes | IM | 2 |
| 317b | Seq. ID No. 655 | Seq. ID No. 656 | maybe | IM | 4 |
| 318a | Seq. ID No. 657 | Seq. ID No. 658 | YES | OM | 2 |
| 318b | Seq. ID No. 659 | Seq. ID No. 660 | YES | OM | 2 |
| 318c | Seq. ID No. 661 | Seq. ID No. 662 | YES | IM | 2 |
| 320 | Seq. ID No. 667 | Seq. ID No. 668 | maybe | Cyto | 1 |
| 321 | Seq. ID No. 669 | Seq. ID No. 670 | yes | OM | 1 |
| 324 | Seq. ID No. 675 | Seq. ID No. 676 | YES | OM | 1 |
| 327a | Seq. ID No. 677 | Seq. ID No. 678 | maybe | IM | 3 |
| 327b | Seq. ID No. 679 | Seq. ID No. 680 | yes | IM | 5 |
| 342a | Seq. ID No. 715 | Seq. ID No. 716 | YES | IM | 2 |
| 342b | Seq. ID No. 717 | Seq. ID No. 718 | YES | IM | 3 |
| 342c | Seq. ID No. 719 | Seq. ID No. 720 | yes | IM | 4 |
| 342d | Seq. ID No. 721 | Seq. ID No. 722 | NO | IM | 6 |
| 342e | Seq. ID No. 723 | Seq. ID No. 724 | yes | IM | 10 |
| 342f | Seq. ID No. 725 | Seq. ID No. 726 | NO | IM | 12 |
| 349b | Seq. ID No. 745 | Seq. ID No. 746 | YES | OM | 1 |
| 349c | Seq. ID No. 747 | Seq. ID No. 748 | YES | OM | 1 |
| 350a | Seq. ID No. 749 | Seq. ID No. 750 | yes | IM | 6 |
| 350b | Seq. ID No. 751 | Seq. ID No. 752 | yes | IM | 6 |
| 352a | Seq. ID No. 755 | Seq. ID No. 756 | YES | IM | 3 |
| 352b | Seq. ID No. 757 | Seq. ID No. 758 | YES | IM | 3 |
| 352c | Seq. ID No. 759 | Seq. ID No. 760 | YES | IM | 6 |
| 354a | Seq. ID No. 761 | Seq. ID No. 762 | YES | IM | 4 |
| 354b | Seq. ID No. 763 | Seq. ID No. 764 | yes | IM | 6 |
| 354c | Seq. ID No. 765 | Seq. ID No. 766 | yes | IM | 6 |
| 356a | Seq. ID No. 769 | Seq. ID No. 770 | yes | IM | 3 |
| 358a | Seq. ID No. 777 | Seq. ID No. 778 | yes | IM | 3 |
| 358b | Seq. ID No. 779 | Seq. ID No. 780 | yes | IM | 10 |
| 358c | Seq. ID No. 781 | Seq. ID No. 782 | yes | IM | 10 |
| 372d | Seq. ID No. 807 | Seq. ID No. 808 | YES | OM | 1 |
| 374 | Seq. ID No. 809 | Seq. ID No. 810 | NO | IM | 1 |
| 382b | Seq. ID No. 819 | Seq. ID No. 820 | yes | OM | 1 |
| 384a | Seq. ID No. 825 | Seq. ID No. 826 | maybe | IM | 2 |
| 403b | Seq. ID No. 863 | Seq. ID No. 864 | NO | Peri | 2 |
| 418a | Seq. ID No. 893 | Seq. ID No. 894 | NO | IM | 5 |
| 419c | Seq. ID No. 901 | Seq. ID No. 902 | NO | IM | 2 |
| 431 | Seq. ID No. 935 | Seq. ID No. 936 | yes | OM | 1 |
| 434 | Seq. ID No. 937 | Seq. ID No. 938 | yes | IM | 2 |
| 437b | Seq. ID No. 941 | Seq. ID No. 942 | maybe | Cyto | 1 |
| 437c | Seq. ID No. 943 | Seq. ID No. 944 | maybe | Cyto | 1 |
| 441b | Seq. ID No. 955 | Seq. ID No. 956 | yes | IM | 2 |
| 443b | Seq. ID No. 957 | Seq. ID No. 958 | maybe | Cyto | |
| 448 | Seq. ID No. 967 | Seq. ID No. 968 | YES | IM | 1 |
| 452b | Seq. ID No. 979 | Seq. ID No. 980 | yes | IM | 4 |
| 452c | Seq. ID No. 981 | Seq. ID No. 982 | yes | IM | 4 |
| 452d | Seq. ID No. 983 | Seq. ID No. 984 | yes | IM | 4 |
| 452e | Seq. ID No. 985 | Seq. ID No. 986 | yes | IM | 4 |
| 460b | Seq. ID No. 1001 | Seq. ID No. 1002 | maybe | Cyto | 1 |
| 460c | Seq. ID No. 1003 | Seq. ID No. 1004 | maybe | Cyto | 1 |
| 462 | Seq. ID No. 1005 | Seq. ID No. 1006 | yes | Peri | 1 |
| 463a | Seq. ID No. 1007 | Seq. ID No. 1008 | yes | Peri | 1 |
| 463b | Seq. ID No. 1009 | Seq. ID No. 1010 | yes | Peri | 1 |
| 464a | Seq. ID No. 1011 | Seq. ID No. 1012 | maybe | IM | 2 |
| 464b | Seq. ID No. 1013 | Seq. ID No. 1014 | YES | IM | 9 |
| 464c | Seq. ID No. 1015 | Seq. ID No. 1016 | yes | IM | 11 |
| 465b | Seq. ID No. 1019 | Seq. ID No. 1020 | yes | OM | 3 |
| 472a | Seq. ID No. 1029 | Seq. ID No. 1030 | yes | IM | 4 |
| 472b | Seq. ID No. 1031 | Seq. ID No. 1032 | yes | IM | 5 |
| 472c | Seq. ID No. 1033 | Seq. ID No. 1034 | maybe | IM | 7 |
| 472d | Seq. ID No. 1035 | Seq. ID No. 1036 | maybe | IM | 8 |
| 472e | Seq. ID No. 1037 | Seq. ID No. 1038 | maybe | IM | 8 |
| 472f | Seq. ID No. 1039 | Seq. ID No. 1040 | YES | IM | 10 |
| 476 | Seq. ID No. 1045 | Seq. ID No. 1046 | maybe | Peri | 1 |
| 480a | Seq. ID No. 1055 | Seq. ID No. 1056 | NO | IM | 1 |
| 492 | Seq. ID No. 1075 | Seq. ID No. 1076 | YES | OM | 1 |
| 494a | Seq. ID No. 1077 | Seq. ID No. 1078 | NO | Cyto | 1 |
| 507a | Seq. ID No. 1105 | Seq. ID No. 1106 | maybe | IM | 1 |
| 507b | Seq. ID No. 1107 | Seq. ID No. 1108 | maybe | IM | 1 |
| 521b | Seq. ID No. 1131 | Seq. ID No. 1132 | maybe | Cyto | 2 |
| 524a | Seq. ID No. 1135 | Seq. ID No. 1136 | maybe | IM | 1 |
| 524b | Seq. ID No. 1137 | Seq. ID No. 1138 | yes | IM | 6 |
| 525 | Seq. ID No. 1139 | Seq. ID No. 1140 | maybe | Cyto | 1 |
| 530a | Seq. ID No. 1151 | Seq. ID No. 1152 | yes | IM | |
| 541a | Seq. ID No. 1165 | Seq. ID No. 1166 | maybe | IM | 2 |
| 541b | Seq. ID No. 1167 | Seq. ID No. 1168 | maybe | IM | 4 |
| 541c | Seq. ID No. 1169 | Seq. ID No. 1170 | yes | IM | 5 |
| 542 | Seq. ID No. 1173 | Seq. ID No. 1174 | yes | IM | 1 |
| 545a | Seq. ID No. 1183 | Seq. ID No. 1184 | yes | IM | 3 |
| 547a | Seq. ID No. 1195 | Seq. ID No. 1196 | YES | OM | 1 |
| 547b | Seq. ID No. 1197 | Seq. ID No. 1198 | YES | IM | 1 |
| 550a | Seq. ID No. 1203 | Seq. ID No. 1204 | maybe | IM | 1 |
| 556 | Seq. ID No. 1213 | Seq. ID No. 1214 | yes | IM | 2 |
| 561 | Seq. ID No. 1223 | Seq. ID No. 1224 | yes | IM | 5 |
| 564a | Seq. ID No. 1227 | Seq. ID No. 1228 | NO | IM | 4 |
| 564b | Seq. ID No. 1229 | Seq. ID No. 1230 | yes | IM | 4 |
| 570b | Seq. ID No. 1237 | Seq. ID No. 1238 | NO | IM | 5 |
| 576a | Seq. ID No. 1243 | Seq. ID No. 1244 | YES | Peri | 1 |
| 576b | Seq. ID No. 1245 | Seq. ID No. 1246 | YES | IM | 1 |
| 585b | Seq. ID No. 1259 | Seq. ID No. 1260 | yes | Cyto | 0 |
| 587a | Seq. ID No. 1265 | Seq. ID No. 1266 | maybe | IM | 4 |
| 587b | Seq. ID No. 1267 | Seq. ID No. 1268 | NO | IM | 5 |
| 587c | Seq. ID No. 1269 | Seq. ID No. 1270 | YES | IM | 9 |
| 587d | Seq. ID No. 1271 | Seq. ID No. 1272 | NO | IM | 12 |
| 595a | Seq. ID No. 1283 | Seq. ID No. 1284 | yes | IM | 2 |
| 596a | Seq. ID No. 1287 | Seq. ID No. 1288 | maybe | IM | 1 |
| 596b | Seq. ID No. 1289 | Seq. ID No. 1290 | maybe | IM | 2 |
| 596c | Seq. ID No. 1291 | Seq. ID No. 1292 | maybe | IM | 2 |
| 598a | Seq. ID No. 1295 | Seq. ID No. 1296 | maybe | IM | 2 |
| 598b | Seq. ID No. 1297 | Seq. ID No. 1298 | maybe | IM | 2 |
| 600a | Seq. ID No. 1303 | Seq. ID No. 1304 | maybe | Peri | 1 |
| 600b | Seq. ID No. 1305 | Seq. ID No. 1306 | maybe | Peri | 1 |
| 609a | Seq. ID No. 1315 | Seq. ID No. 1316 | YES | IM | 2 |
| 609b | Seq. ID No. 1317 | Seq. ID No. 1318 | YES | IM | 2 |
| 610a | Seq. ID No. 1319 | Seq. ID No. 1320 | yes | IM | 1 |
| 613a | Seq. ID No. 1325 | Seq. ID No. 1326 | maybe | IM | 6 |
| 613b | Seq. ID No. 1327 | Seq. ID No. 1328 | yes | IM | 7 |
| 613c | Seq. ID No. 1329 | Seq. ID No. 1330 | NO | IM | 9 |
| 613d | Seq. ID No. 1331 | Seq. ID No. 1332 | YES | IM | 10 |
| 615b | Seq. ID No. 1337 | Seq. ID No. 1338 | maybe | IM | 2 |
| 619a | Seq. ID No. 1341 | Seq. ID No. 1342 | yes | Cyto | 1 |
| 619b | Seq. ID No. 1343 | Seq. ID No. 1344 | yes | Cyto | 1 |
| 623 | Seq. ID No. 1347 | Seq. ID No. 1348 | yes | Peri | 1 |
| 624a | Seq. ID No. 1349 | Seq. ID No. 1350 | NO | IM | 1 |
| 624b | Seq. ID No. 1351 | Seq. ID No. 1352 | maybe | IM | 1 |
| 624c | Seq. ID No. 1353 | Seq. ID No. 1354 | maybe | IM | 1 |
| 631b | Seq. ID No. 1365 | Seq. ID No. 1366 | YES | IM | 2 |
| 631c | Seq. ID No. 1367 | Seq. ID No. 1368 | YES | IM | 2 |
| 631d | Seq. ID No. 1369 | Seq. ID No. 1370 | YES | IM | 2 |
| 632b | Seq. ID No. 1371 | Seq. ID No. 1372 | maybe | IM | 1 |
| 636 | Seq. ID No. 1381 | Seq. ID No. 1382 | maybe | Cyto | |
| 645 | Seq. ID No. 1401 | Seq. ID No. 1402 | yes | Peri | 1 |
| 665 | Seq. ID No. 1429 | Seq. ID No. 1430 | yes | IM | 3 |
| 674a | Seq. ID No. 1435 | Seq. ID No. 1436 | NO | IM | 1 |
| 679a | Seq. ID No. 1445 | Seq. ID No. 1446 | YES | Cyto | 1 |
| 679b | Seq. ID No. 1447 | Seq. ID No. 1448 | YES | IM | 1 |
| 700a | Seq. ID No. 1481 | Seq. ID No. 1482 | NO | IM | 2 |
| 722a | Seq. ID No. 1513 | Seq. ID No. 1514 | YES | IM | 3 |
| 722b | Seq. ID No. 1515 | Seq. ID No. 1516 | maybe | IM | 7 |
| 722c | Seq. ID No. 1517 | Seq. ID No. 1518 | YES | IM | 11 |
| 728a | Seq. ID No. 1529 | Seq. ID No. 1530 | NO | IM | 6 |
| 730a | Seq. ID No. 1533 | Seq. ID No. 1534 | NO | IM | 3 |
| 730b | Seq. ID No. 1535 | Seq. ID No. 1536 | maybe | IM | 5 |
| 738a | Seq. ID No. 1545 | Seq. ID No. 1546 | YES | IM | 2 |
| 738b | Seq. ID No. 1547 | Seq. ID No. 1548 | NO | IM | 9 |
| 739 | Seq. ID No. 1551 | Seq. ID No. 1552 | yes | IM | 2 |
| 740 | Seq. ID No. 1553 | Seq. ID No. 1554 | NO | OM | 0 |
| 743a | Seq. ID No. 1559 | Seq. ID No. 1560 | yes | IM | 2 |
| 743b | Seq. ID No. 1561 | Seq. ID No. 1562 | YES | IM | 2 |
| 744 | Seq. ID No. 1563 | Seq. ID No. 1564 | maybe | OM | 1 |
| 746b | Seq. ID No. 1571 | Seq. ID No. 1572 | YES | IM | 6 |
| 749 | Seq. ID No. 1579 | Seq. ID No. 1580 | yes | IM | 1 |
| 754a | Seq. ID No. 1583 | Seq. ID No. 1584 | NO | IM | 3 |
| 754b | Seq. ID No. 1585 | Seq. ID No. 1586 | YES | IM | 9 |
| 772a | Seq. ID No. 1621 | Seq. ID No. 1622 | yes | IM | 5 |
| 779 | Seq. ID No. 1633 | Seq. ID No. 1634 | yes | Peri | 2 |
| 782b | Seq. ID No. 1639 | Seq. ID No. 1640 | NO | IM | 5 |
| 786b | Seq. ID No. 1643 | Seq. ID No. 1644 | YES | Peri | 1 |
| 788a | Seq. ID No. 1645 | Seq. ID No. 1646 | yes | IM | 4 |
| 788b | Seq. ID No. 1647 | Seq. ID No. 1648 | yes | IM | 6 |
| 789a | Seq. ID No. 1649 | Seq. ID No. 1650 | yes | IM | 4 |
| 789c | Seq. ID No. 1653 | Seq. ID No. 1654 | YES | IM | 5 |
| 794b | Seq. ID No. 1661 | Seq. ID No. 1662 | YES | OM | 0 |
| 794c | Seq. ID No. 1663 | Seq. ID No. 1664 | YES | OM | 0 |
| 794d | Seq. ID No. 1665 | Seq. ID No. 1666 | yes | OM | 0 |
| 799a | Seq. ID No. 1671 | Seq. ID No. 1672 | yes | IM | 2 |
| 799b | Seq. ID No. 1673 | Seq. ID No. 1674 | maybe | IM | 4 |
| 799d | Seq. ID No. 1677 | Seq. ID No. 1678 | yes | IM | 9 |
| 799e | Seq. ID No. 1679 | Seq. ID No. 1680 | yes | IM | 10 |
| 799f | Seq. ID No. 1681 | Seq. ID No. 1682 | yes | IM | 10 |
| 808 | Seq. ID No. 1693 | Seq. ID No. 1694 | NO | Cyto | |
| 826 | Seq. ID No. 1707 | Seq. ID No. 1708 | YES | IM | 3 |
| 829b | Seq. ID No. 1711 | Seq. ID No. 1712 | NO | Peri | |
| 837a | Seq. ID No. 1731 | Seq. ID No. 1732 | yes | IM | 2 |
| 837b | Seq. ID No. 1733 | Seq. ID No. 1734 | YES | IM | 8 |
| 843 | Seq. ID No. 1737 | Seq. ID No. 1738 | YES | IM | 1 |
| 853a | Seq. ID No. 1753 | Seq. ID No. 1754 | yes | IM | 3 |
| 853b | Seq. ID No. 1755 | Seq. ID No. 1756 | YES | IM | 5 |
| 853c | Seq. ID No. 1757 | Seq. ID No. 1758 | maybe | IM | 5 |
| 859a | Seq. ID No. 1765 | Seq. ID No. 1766 | YES | IM | 2 |
| 859b | Seq. ID No. 1767 | Seq. ID No. 1768 | YES | IM | 4 |
| 859d | Seq. ID No. 1771 | Seq. ID No. 1772 | NO | IM | 6 |
| 864b | Seq. ID No. 1781 | Seq. ID No. 1782 | maybe | Cyto | 1 |
| 868a | Seq. ID No. 1787 | Seq. ID No. 1788 | yes | IM | 3 |
| 870 | Seq. ID No. 1791 | Seq. ID No. 1792 | YES | OM | 1 |
| 873c | Seq. ID No. 1797 | Seq. ID No. 1798 | yes | OM | 1 |
| 876 | Seq. ID No. 1805 | Seq. ID No. 1806 | YES | Peri | 1 |
| 885 | Seq. ID No. 1819 | Seq. ID No. 1820 | NO | IM | 2 |
| 886 | Seq. ID No. 1821 | Seq. ID No. 1822 | yes | OM | 1 |
| 887 | Seq. ID No. 1823 | Seq. ID No. 1824 | yes | IM | 1 |
| 908a | Seq. ID No. 1839 | Seq. ID No. 1840 | yes | IM | 6 |
| 919 | Seq. ID No. 1857 | Seq. ID No. 1858 | maybe | Cyto | |
| 929a | Seq. ID No. 1869 | Seq. ID No. 1870 | maybe | Cyto | 1 |
| 931 | Seq. ID No. 1875 | Seq. ID No. 1876 | YES | IM | 2 |
| 939a | Seq. ID No. 1891 | Seq. ID No. 1892 | maybe | IM | 9 |
| 939b | Seq. ID No. 1893 | Seq. ID No. 1894 | yes | IM | 10 |
| 939c | Seq. ID No. 1895 | Seq. ID No. 1896 | NO | IM | 12 |
| 950 | Seq. ID NO. 1915 | Seq. ID No. 1916 | NO | IM | 1 |
| 951a | Seq. ID No. 1917 | Seq. ID No. 1918 | yes | OM | 0 |
| 951b | Seq. ID No. 1919 | Seq. ID No. 1920 | yes | IM | 0 |
| 952b | Seq. ID No. 1923 | Seq. ID No. 1924 | maybe | IM | 4 |
| 955a | Seq. ID No. 1927 | Seq. ID No. 1928 | maybe | IM | 1 |
| 955b | Seq. ID No. 1929 | Seq. ID No. 1930 | maybe | IM | 1 |
| 977a | Seq. ID No. 1969 | Seq. ID No. 1970 | YES | OM | 1 |
| 981a | Seq. ID No. 1981 | Seq. ID No. 1982 | YES | IM | 1 |
| 987 | Seq. ID No. 1991 | Seq. ID No. 1992 | yes | IM | 5 |
| 988a | Seq. ID No. 1993 | Seq. ID No. 1994 | NO | IM | 5 |
| 993 | Seq. ID No. 2005 | Seq. ID No. 2006 | maybe | IM | 0 |
| 998a | Seq. ID No. 2013 | Seq. ID No. 2014 | yes | IM | 5 |
| 999a | Seq. ID No. 2017 | Seq. ID No. 2018 | yes | IM | 2 |
| 999b | Seq. ID No. 2019 | Seq. ID No. 2020 | yes | IM | 2 |
| 1003a | Seq. ID No. 2025 | Seq. ID No. 2026 | maybe | IM | 2 |
| 1003b | Seq. ID No. 2027 | Seq. ID No. 2028 | maybe | IM | 2 |
| 1006a | Seq. ID No. 2033 | Seq. ID No. 2034 | yes | Peri | 1 |
| 1006b | Seq. ID No. 2035 | Seq. ID No. 2036 | yes | Cyto | 1 |
| 1013a | Seq. ID No. 2041 | Seq. ID No. 2042 | maybe | IM | 5 |
| 1017a | Seq. ID No. 2049 | Seq. ID No. 2050 | yes | IM | 4 |
| 1017c | Seq. ID No. 2053 | Seq. ID No. 2054 | YES | IM | 6 |
| 1017d | Seq. ID No. 2055 | Seq. ID No. 2056 | YES | IM | 6 |
| 1036a | Seq. ID No. 2087 | Seq. ID No. 2088 | YES | IM | 3 |
| 1036b | Seq. ID No. 2089 | Seq. ID No. 2090 | YES | IM | 4 |
| 1036c | Seq. ID No. 2091 | Seq. ID No. 2092 | YES | IM | 4 |
| 1044a | Seq. ID No. 2105 | Seq. ID No. 2106 | YES | IM | 2 |
| 1044b | Seq. ID No. 2107 | Seq. ID No. 2108 | maybe | IM | 3 |
| 1044c | Seq. ID No. 2109 | Seq. ID No. 2110 | maybe | IM | 3 |
| 1047a | Seq. ID No. 2113 | Seq. ID No. 2114 | yes | OM | 1 |
| 1047b | Seq. ID No. 2115 | Seq. ID No. 2116 | YES | OM | 1 |
| 1050a | Seq. ID No. 2121 | Seq. ID No. 2122 | maybe | IM | 3 |
| 1050b | Seq. ID No. 2123 | Seq. ID No. 2124 | yes | IM | 7 |
| 1050c | Seq. ID No. 2125 | Seq. ID No. 2126 | yes | IM | 7 |
| 1050d | Seq. ID No. 2127 | Seq. ID No. 2128 | yes | IM | 7 |
| 1065a | Seq. ID No. 2149 | Seq. ID No. 2150 | YES | Peri | 1 |
| 1065b | Seq. ID No. 2151 | Seq. ID No. 2152 | YES | Peri | 1 |
| 1066a | Seq. ID No. 2155 | Seq. ID No. 2156 | NO | IM | 1 |
| 1066b | Seq. ID No. 2157 | Seq. ID No. 2158 | NO | IM | 1 |
| 1067 | Seq. ID No. 2159 | Seq. ID No. 2160 | YES | OM | 1 |
| 1077a | Seq. ID No. 2171 | Seq. ID No. 2172 | yes | IM | 3 |
| 1077b | Seq. ID No. 2173 | Seq. ID No. 2174 | maybe | IM | 10 |
| 1077d | Seq. ID No. 2177 | Seq. ID No. 2178 | YES | IM | 13 |
| 1081a | Seq. ID No. 2187 | Seq. ID No. 2188 | yes | Peri | 1 |
| 1082 | Seq. ID No. 2191 | Seq. ID No. 2192 | maybe | Peri | 0 |
| 1094a | Seq. ID No. 2205 | Seq. ID No. 2206 | maybe | IM | 2 |
| 1111a | Seq. ID No. 2237 | Seq. ID No. 2238 | yes | IM | 6 |
| 1111b | Seq. ID No. 2239 | Seq. ID No. 2240 | maybe | IM | 8 |
| 1125 | Seq. ID No. 2245 | Seq. ID No. 2246 | NO | IM | 4 |
| 1126b | Seq. ID No. 2249 | Seq. ID No. 2250 | yes | IM | 4 |
| 1126c | Seq. ID No. 2251 | Seq. ID No. 2252 | yes | IM | 4 |
| 1128 | Seq. ID No. 2255 | Seq. ID No. 2256 | yes | IM | 2 |
| 1132 | Seq. ID No. 2259 | Seq. ID No. 2260 | NO | Cyto | |
| 1140a | Seq. ID No. 2271 | Seq. ID No. 2272 | yes | IM | 2 |
| 1144a | Seq. ID No. 2283 | Seq. ID No. 2284 | yes | IM | 2 |
| 1146a | Seq. ID No. 2289 | Seq. ID No. 2290 | yes | IM | 3 |
| 1146b | Seq. ID No. 2291 | Seq. ID No. 2292 | NO | IM | 5 |
| 1146c | Seq. ID No. 2293 | Seq. ID No. 2294 | YES | IM | 7 |
| 1146d | Seq. ID No. 2295 | Seq. ID No. 2296 | yes | IM | 8 |
| 1152a | Seq. ID No. 2303 | Seq. ID No. 2304 | maybe | IM | 2 |
| 1152b | Seq. ID No. 2305 | Seq. ID No. 2306 | yes | IM | 5 |
| 1152c | Seq. ID No. 2307 | Seq. ID No. 2308 | yes | IM | 6 |
| 1155a | Seq. ID No. 2317 | Seq. ID No. 2318 | yes | IM | 3 |
| 1155b | Seq. ID No. 2319 | Seq. ID No. 2320 | yes | IM | 4 |
| 1155c | Seq. ID No. 2321 | Seq. ID No. 2322 | YES | IM | 5 |
| 1155d | Seq. ID No. 2323 | Seq. ID No. 2324 | yes | IM | 6 |
| 1155e | Seq. ID No. 2325 | Seq. ID No. 2326 | yes | IM | 9 |
| 1155f | Seq. ID No. 2327 | Seq. ID No. 2328 | maybe | IM | 11 |
| 1155g | Seq. ID No. 2329 | Seq. ID No. 2330 | maybe | IM | 11 |
| 1158b | Seq. ID No. 2333 | Seq. ID No. 2334 | maybe | Cyto | 1 |
| 1165 | Seq. ID No. 2343 | Seq. ID No. 2344 | yes | OM | 1 |
| 1167a | Seq. ID No. 2347 | Seq. ID No. 2348 | YES | Peri | 1 |
| 1167b | Seq. ID No. 2349 | Seq. ID No. 2350 | yes | IM | 2 |
| 1168 | Seq. ID No. 2351 | Seq. ID No. 2352 | YES | Peri | 1 |
| 1169a | Seq. ID No. 2353 | Seq. ID No. 2354 | YES | IM | 4 |
| 1169b | Seq. ID No. 2355 | Seq. ID No. 2356 | maybe | IM | 6 |
| 1169c | Seq. ID No. 2357 | Seq. ID No. 2358 | maybe | IM | 8 |
| 1172 | Seq. ID No. 2365 | Seq. ID No. 2366 | YES | IM | 1 |
| 1173 | Seq. ID No. 2367 | Seq. ID No. 2368 | maybe | IM | 2 |
| 1175 | Seq. ID No. 2371 | Seq. ID No. 2372 | maybe | Cyto | 0 |
| 1178 | Seq. ID No. 2375 | Seq. ID No. 2376 | maybe | Cyto | 1 |
| 1180c | Seq. ID No. 2387 | Seq. ID No. 2388 | maybe | IM | 3 |
| 1180d | Seq. ID No. 2389 | Seq. ID No. 2390 | maybe | IM | 3 |
| 1180e | Seq. ID No. 2391 | Seq. ID No. 2392 | maybe | IM | 4 |
| 1182a | Seq. ID No. 2393 | Seq. ID No. 2394 | yes | IM | 1 |
| 1186a | Seq. ID No. 2407 | Seq. ID No. 2408 | maybe | IM | 5 |
| 1192a | Seq. ID No. 2419 | Seq. ID No. 2420 | maybe | IM | 3 |
| 1192b | Seq. ID No. 2421 | Seq. ID No. 2422 | YES | IM | 4 |
| 1196a | Seq. ID No. 2425 | Seq. ID No. 2426 | YES | Peri | 1 |
| 1196b | Seq. ID No. 2427 | Seq. ID No. 2428 | YES | Peri | 1 |
| 1197 | Seq. ID No. 2429 | Seq. ID No. 2430 | YES | IM | 2 |
| 1199a | Seq. ID No. 2433 | Seq. ID No. 2434 | maybe | IM | 3 |
| 1207 | Seq. ID No. 2449 | Seq. ID No. 2450 | YES | OM | 1 |
| 1212 | Seq. ID No. 2459 | Seq. ID No. 2460 | YES | IM | 2 |
| 1224a | Seq. ID No. 2487 | Seq. ID No. 2488 | maybe | IM | 6 |
| 1224b | Seq. ID No. 2489 | Seq. ID No. 2490 | maybe | IM | 6 |
| 1224c | Seq. ID No. 2491 | Seq. ID No. 2492 | yes | IM | 6 |
| 1225 | Seq. ID No. 2493 | Seq. ID No. 2494 | NO | Cyto | 1 |
| 1228a | Seq. ID No. 2497 | Seq. ID No. 2498 | yes | IM | 1 |
| 1228b | Seq. ID No. 2499 | Seq. ID No. 2500 | yes | IM | 2 |
| 1229b | Seq. ID No. 2501 | Seq. ID No. 2502 | NO | Cyto | 2 |
| 1229c | Seq. ID No. 2503 | Seq. ID No. 2504 | NO | Cyto | 2 |
| 1231 | Seq. ID No. 2507 | Seq. ID No. 2508 | yes | OM | 1 |
| 1234a | Seq. ID No. 2515 | Seq. ID No. 2516 | yes | IM | 4 |
| 1234b | Seq. ID No. 2517 | Seq. ID No. 2518 | yes | IM | 4 |
| 1236a | Seq. ID No. 2523 | Seq. ID No. 2524 | yes | IM | 2 |
| 1237 | Seq. ID No. 2525 | Seq. ID No. 2526 | YES | OM | 1 |
| 1239 | Seq. ID No. 2527 | Seq. ID No. 2528 | yes | OM | 1 |
| 1241a | Seq. ID No. 2531 | Seq. ID No. 2532 | YES | IM | 2 |
| 1241b | Seq. ID No. 2533 | Seq. ID No. 2534 | yes | IM | 7 |
| 1241c | Seq. ID No. 2535 | Seq. ID No. 2536 | yes | IM | 7 |
| 1243 | Seq. ID No. 2537 | Seq. ID No. 2538 | maybe | IM | 4 |
| 1245a | Seq. ID No. 2541 | Seq. ID No. 2542 | yes | IM | 6 |
| 1245b | Seq. ID No. 2543 | Seq. ID No. 2544 | yes | IM | 10 |
| 1245c | Seq. ID No. 2545 | Seq. ID No. 2546 | maybe | IM | 11 |
| 1250a | Seq. ID No. 2555 | Seq. ID No. 2556 | maybe | IM | 2 |
| 1250b | Seq. ID No. 2557 | Seq. ID No. 2558 | yes | IM | 8 |
| 1251a | Seq. ID No. 2565 | Seq. ID No. 2566 | maybe | IM | 3 |
| 1253a | Seq. ID No. 2569 | Seq. ID No. 2570 | maybe | OM | 1 |
| 1253b | Seq. ID No. 2571 | Seq. ID No. 2572 | maybe | OM | 2 |
| 1258a | Seq. ID No. 2583 | Seq. ID No. 2584 | yes | IM | 5 |
| 1260 | Seq. ID No. 2591 | Seq. ID No. 2592 | maybe | Cyto | 1 |
| 1266a | Seq. ID No. 2597 | Seq. ID No. 2598 | NO | IM | 6 |
| 1266b | Seq. ID No. 2599 | Seq. ID No. 2600 | maybe | IM | 7 |
| 1267a | Seq. ID No. 2601 | Seq. ID No. 2602 | yes | IM | 2 |
| 1268b | Seq. ID No. 2607 | Seq. ID No. 2608 | YES | IM | 5 |
| 1270 | Seq. ID No. 2611 | Seq. ID No. 2612 | maybe | IM | 2 |
| 1275a | Seq. ID No. 2621 | Seq. ID No. 2622 | yes | Cyto | 1 |
| 1278a | Seq. ID No. 2627 | Seq. ID No. 2628 | NO | IM | 1 |
| 1278b | Seq. ID No. 2629 | Seq. ID No. 2630 | NO | IM | 2 |
| 1288 | Seq. ID No. 2651 | Seq. ID No. 2652 | YES | IM | 1 |
| 1308a | Seq. ID No. 2679 | Seq. ID No. 2680 | YES | IM | 3 |
| 1308b | Seq. ID No. 2681 | Seq. ID No. 2682 | yes | IM | 4 |
| 1308c | Seq. ID No. 2683 | Seq. ID No. 2684 | maybe | IM | 4 |
| 1314b | Seq. ID No. 2691 | Seq. ID No. 2692 | yes | IM | 1 |
| 1315 | Seq. ID No. 2693 | Seq. ID No. 2694 | YES | IM | 2 |
| 1316 | Seq. ID No. 2695 | Seq. ID No. 2696 | maybe | IM | |
| 1317 | Seq. ID No. 2697 | Seq. ID No. 2698 | NO | Cyto | 1 |
| 1318 | Seq. ID No. 2699 | Seq. ID No. 2700 | YES | IM | 0 |
| 1319a | Seq. ID No. 2701 | Seq. ID No. 2702 | yes | IM | 4 |
| 1319b | Seq. ID No. 2703 | Seq. ID No. 2704 | YES | IM | 5 |
| 1320a | Seq. ID No. 2705 | Seq. ID No. 2706 | maybe | IM | 2 |
| 1320b | Seq. ID No. 2707 | Seq. ID No. 2708 | NO | IM | 4 |
| 1323b | Seq. ID No. 2717 | Seq. ID No. 2718 | maybe | OM | 1 |
| 1334a | Seq. ID No. 2729 | Seq. ID No. 2730 | maybe | IM | 2 |
| 1334b | Seq. ID No. 2731 | Seq. ID No. 2732 | maybe | OM | 2 |
| 1335a | Seq. ID No. 2733 | Seq. ID No. 2734 | yes | OM | 2 |
| 1365b | Seq. ID No. 2783 | Seq. ID No. 2784 | maybe | IM | 2 |
| 1366a | Seq. ID No. 2785 | Seq. ID No. 2786 | YES | Peri | 1 |
| 1375 | Seq. ID No. 2801 | Seq. ID No. 2802 | yes | Cyto | 0 |
| 1380 | Seq. ID No. 2809 | Seq. ID No. 2810 | NO | IM | 2 |
| 1381b | Seq. ID No. 2813 | Seq. ID No. 2814 | YES | IM | 2 |
| 1382 | Seq. ID No. 2815 | Seq. ID No. 2816 | yes | OM | 1 |
| 1384a | Seq. ID No. 2819 | Seq. ID No. 2820 | yes | IM | 2 |
| 1384b | Seq. ID No. 2821 | Seq. ID No. 2822 | yes | IM | 2 |
| 1384c | Seq. ID No. 2823 | Seq. ID No. 2824 | yes | IM | 2 |
| 1388b | Seq. ID No. 2831 | Seq. ID No. 2832 | maybe | Cyto | |
| 1389a | Seq. ID No. 2833 | Seq. ID No. 2834 | yes | Cyto | 0 |
| 1393a | Seq. ID No. 2835 | Seq. ID No. 2836 | YES | Peri | 1 |
| 1393b | Seq. ID No. 2837 | Seq. ID No. 2838 | yes | IM | 2 |
| 1393c | Seq. ID No. 2839 | Seq. ID No. 2840 | yes | IM | 3 |
| 1393d | Seq. ID No. 2841 | Seq. ID No. 2842 | YES | IM | 3 |
| 1395a | Seq. ID No. 2845 | Seq. ID No. 2846 | yes | Cyto | |
| 1399a | Seq. ID No. 2853 | Seq. ID No. 2854 | yes | IM | 2 |
| 1399b | Seq. ID No. 2855 | Seq. ID No. 2856 | maybe | IM | 2 |
| 1401 | Seq. ID No. 2859 | Seq. ID No. 2860 | NO | IM | 1 |
| 1402c | Seq. ID No. 2861 | Seq. ID No. 2862 | yes | IM | 2 |
| 1411a | Seq. ID No. 2873 | Seq. ID No. 2874 | yes | IM | 3 |
| 1412b | Seq. ID No. 2881 | Seq. ID No. 2882 | YES | IM | 9 |
| 1418 | Seq. ID No. 2893 | Seq. ID No. 2894 | maybe | Cyto | |
| 1426a | Seq. ID No. 2905 | Seq. ID No. 2906 | yes | IM | 4 |
| 1438 | Seq. ID No. 2935 | Seq. ID No. 2936 | YES | OM | 1 |
| 1439a | Seq. ID No. 2937 | Seq. ID No. 2938 | YES | IM | 3 |
| 1439b | Seq. ID No. 2939 | Seq. ID No. 2940 | yes | IM | 5 |
| 1439c | Seq. ID No. 2941 | Seq. ID No. 2942 | yes | IM | 6 |
| 1440a | Seq. ID No. 2947 | Seq. ID No. 2948 | yes | IM | 6 |
| 1440b | Seq. ID No. 2949 | Seq. ID No. 2950 | YES | IM | 10 |
| 1440c | Seq. ID No. 2951 | Seq. ID No. 2952 | maybe | IM | 12 |
| 1447a | Seq. ID No. 2969 | Seq. ID No. 2970 | NO | IM | 10 |
| 1448 | Seq. ID No. 2973 | Seq. ID No. 2974 | YES | IM | 1 |
| 1449a | Seq. ID No. 2975 | Seq. ID No. 2976 | maybe | OM | 1 |
| 1449b | Seq. ID No. 2977 | Seq. ID No. 2978 | maybe | OM | 1 |
| 1452a | Seq. ID No. 2981 | Seq. ID No. 2982 | yes | IM | 4 |
| 1452b | Seq. ID No. 2983 | Seq. ID No. 2984 | yes | IM | 4 |
| 1454 | Seq. ID No. 2987 | Seq. ID No. 2988 | yes | Cyto | 1 |
| 1455b | Seq. ID No. 2991 | Seq. ID No. 2992 | yes | OM | 1 |
| 1457a | Seq. ID No. 2995 | Seq. ID No. 2996 | yes | IM | 3 |
| 1457b | Seq. ID No. 2997 | Seq. ID No. 2998 | maybe | IM | 4 |
| 1462b | Seq. ID No. 3005 | Seq. ID No. 3006 | yes | IM | 6 |
| 1463a | Seq. ID No. 3007 | Seq. ID No. 3008 | YES | IM | 2 |
| 1463b | Seq. ID No. 3009 | Seq. ID No. 3010 | YES | IM | 2 |
| 14630 | Seq. ID No. 3011 | Seq. ID No. 3012 | YES | IM | 2 |
| 1466a | Seq. ID No. 3013 | Seq. ID No. 3014 | maybe | IM | 1 |
| 1469a | Seq. ID No. 3023 | Seq. ID No. 3024 | NO | IM | 4 |
| 1469b | Seq. ID No. 3025 | Seq. ID No. 3026 | yes | IM | 5 |
| 1469c | Seq. ID No. 3027 | Seq. ID No. 3028 | maybe | IM | 11 |
| 1470a | Seq. ID No. 3029 | Seq. ID No. 3030 | YES | IM | 5 |
| 1470b | Seq. ID No. 3031 | Seq. ID No. 3032 | YES | IM | 7 |
| 1475 | Seq. ID No. 3043 | Seq. ID No. 3044 | yes | OM | 1 |
| 1479c | Seq. ID No. 3055 | Seq. ID No. 3056 | NO | Peri | 1 |
| 1484a | Seq. ID No. 3063 | Seq. ID No. 3064 | maybe | IM | 4 |
| 1487 | Seq. ID No. 3071 | Seq. ID No. 3072 | maybe | OM | 2 |
| 1488a | Seq. ID No. 3073 | Seq. ID No. 3074 | yes | IM | 1 |
| 1488b | Seq. ID No. 3075 | Seq. ID No. 3076 | yes | IM | 2 |
| 1488c | Seq. ID No. 3077 | Seq. ID No. 3078 | maybe | IM | 3 |
| 1507a | Seq. ID No. 3119 | Seq. ID No. 3120 | NO | IM | 2 |
| 1507b | Seq. ID No. 3121 | Seq. ID No. 3122 | NO | IM | 2 |
| 1507c | Seq. ID No. 3123 | Seq. ID No. 3124 | maybe | IM | 2 |
| 1517a | Seq. ID No. 3145 | Seq. ID No. 3146 | NO | Peri | 1 |
| 1520 | Seq. ID No. 3155 | Seq. ID No. 3156 | yes | Peri | 1 |
| 1522 | Seq. ID No. 3157 | Seq. ID No. 3158 | NO | Peri | 1 |
| 1530a | Seq. ID No. 3163 | Seq. ID No. 3164 | yes | OM | 1 |
| 1530b | Seq. ID No. 3165 | Seq. ID No. 3166 | yes | IM | 1 |
| 1535a | Seq. ID No. 3173 | Seq. ID No. 3174 | maybe | Cyto | 1 |
| 1543 | Seq. ID No. 3183 | Seq. ID No. 3184 | yes | OM | 1 |
| 1552 | Seq. ID No. 3193 | Seq. ID No. 3194 | maybe | IM | 2 |
| 1554b | Seq. ID No. 3201 | Seq. ID No. 3202 | maybe | IM | 2 |
| 1557a | Seq. ID No. 3213 | Seq. ID No. 3214 | yes | IM | 4 |
| 1557b | Seq. ID No. 3215 | Seq. ID No. 3216 | yes | IM | 5 |
| 1557c | Seq. ID No. 3217 | Seq. ID No. 3218 | maybe | IM | 6 |
| 1559a | Seq. ID No. 3219 | Seq. ID No. 3220 | YES | IM | 4 |
| 1572a | Seq. ID No. 3241 | Seq. ID No. 3242 | maybe | IM | 2 |
| 1572b | Seq. ID No. 3243 | Seq. ID No. 3244 | NO | IM | 8 |
| 1572c | Seq. ID No. 3245 | Seq. ID No. 3246 | yes | IM | 10 |
| 1572e | Seq. ID No. 3249 | Seq. ID No. 3250 | NO | IM | 12 |
| 1577a | Seq. ID No. 3255 | Seq. ID No. 3256 | maybe | IM | 4 |
| 1577b | Seq. ID No. 3257 | Seq. ID No. 3258 | maybe | IM | 4 |
| 1580a | Seq. ID No. 3261 | Seq. ID No. 3262 | yes | IM | 1 |
| 1580b | Seq. ID No. 3263 | Seq. ID No. 3264 | yes | IM | 1 |
| 1582b | Seq. ID No. 3269 | Seq. ID No. 3270 | yes | IM | 1 |
| 1585a | Seq. ID No. 3275 | Seq. ID No. 3276 | yes | IM | 1 |
| 1585b | Seq. ID No. 3277 | Seq. ID No. 3278 | yes | IM | 1 |
| 1594a | Seq. ID No. 3297 | Seq. ID No. 3298 | YES | IM | 1 |
| 1606a | Seq. ID No. 3317 | Seq. ID No. 3318 | yes | IM | 1 |
| 1607b | Seq. ID No. 3323 | Seq. ID No. 3324 | maybe | IM | 6 |
| 1608 | Seq. ID No. 3325 | Seq. ID No. 3326 | yes | Cyto | 1 |
| 1612 | Seq. ID No. 3331 | Seq. ID No. 3332 | YES | IM | 3 |
| 1616b | Seq. ID No. 3335 | Seq. ID No. 3336 | YES | IM | 4 |
| 1619 | Seq. ID No. 3337 | Seq. ID No. 3338 | yes | OM | 1 |
| 1621 | Seq. ID No. 3339 | Seq. ID No. 3340 | maybe | IM | 1 |
| 1624 | Seq. ID No. 3349 | Seq. ID No. 3350 | YES | Peri | 1 |
| 1625a | Seq. ID No. 3351 | Seq. ID No. 3352 | yes | IM | 4 |
| 1625b | Seq. ID No. 3353 | Seq. ID No. 3354 | YES | IM | 8 |
| 1625c | Seq. ID No. 3355 | Seq. ID No. 3356 | YES | IM | 8 |
| 1626a | Seq. ID No. 3357 | Seq. ID No. 3358 | maybe | IM | 4 |
| 1626b | Seq. ID No. 3359 | Seq. ID No. 3360 | maybe | IM | 9 |
| 1634b | Seq. ID No. 3375 | Seq. ID No. 3376 | maybe | OM | 0 |
| 1634c | Seq. ID No. 3377 | Seq. ID No. 3378 | YES | IM | 1 |
| 1646a | Seq. ID No. 3389 | Seq. ID No. 3390 | yes | IM | 2 |
| 1646b | Seq. ID No. 3391 | Seq. ID No. 3392 | YES | IM | 6 |
| 1646d | Seq. ID No. 3395 | Seq. ID No. 3396 | maybe | IM | 13 |
| 1649a | Seq. ID No. 3405 | Seq. ID No. 3406 | yes | Peri | 2 |
| 1654 | Seq. ID No. 3415 | Seq. ID No. 3416 | maybe | Peri | 0 |
| 1659a | Seq. ID No. 3421 | Seq. ID No. 3422 | yes | OM | 1 |
| 1659c | Seq. ID No. 3425 | Seq. ID No. 3426 | YES | IM | 10 |
| 1660b | Seq. ID No. 3427 | Seq. ID No. 3428 | maybe | IM | 4 |
| 1663 | Seq. ID No. 3433 | Seq. ID No. 3434 | maybe | Cyto | 0 |
| 1669 | Seq. ID No. 3441 | Seq. ID No. 3442 | maybe | Cyto | 1 |
| 1673a | Seq. ID No. 3449 | Seq. ID No. 3450 | YES | IM | 2 |
| 1673b | Seq. ID No. 3451 | Seq. ID No. 3452 | yes | IM | 8 |
| 1673c | Seq. ID No. 3453 | Seq. ID No. 3454 | yes | IM | 10 |
| 1673d | Seq. ID No. 3455 | Seq. ID No. 3456 | yes | IM | 12 |
| 1673e | Seq. ID No. 3457 | Seq. ID No. 3458 | maybe | IM | 13 |
| 1675a | Seq. ID No. 3463 | Seq. ID No. 3464 | maybe | IM | 1 |
| 1683a | Seq. ID No. 3481 | Seq. ID No. 3482 | maybe | IM | 4 |
| 1688a | Seq. ID No. 3491 | Seq. ID No. 3492 | yes | IM | 3 |
| 1688b | Seq. ID No. 3493 | Seq. ID No. 3494 | YES | IM | 10 |
| 1690a | Seq. ID No. 3503 | Seq. ID No. 3504 | yes | IM | 1 |
| 1690b | Seq. ID No. 3505 | Seq. ID No. 3506 | yes | IM | 2 |
| 1694c | Seq. ID No. 3517 | Seq. ID No. 3518 | yes | IM | 1 |
| 1694d | Seq. ID No. 3519 | Seq. ID No. 3520 | yes | IM | 1 |
| 1699a | Seq. ID No. 3527 | Seq. ID No. 3528 | maybe | IM | 7 |
| 1700a | Seq. ID No. 3531 | Seq. ID No. 3532 | maybe | IM | 4 |
| 1703b | Seq. ID No. 3539 | Seq. ID No. 3540 | NO | Cyto | 0 |
| 1710b | Seq. ID No. 3561 | Seq. ID No. 3562 | NO | Cyto | |
| 1718b | Seq. ID No. 3573 | Seq. ID No. 3574 | YES | Cyto | |
| 1724 | Seq. ID No. 3581 | Seq. ID No. 3582 | maybe | IM | 2 |
| 1731 | Seq. ID No. 3587 | Seq. ID No. 3588 | NO | IM | 2 |
| 1735a | Seq. ID No. 3593 | Seq. ID No. 3594 | yes | OM | 1 |
| 1735b | Seq. ID No. 3595 | Seq. ID No. 3596 | maybe | OM | 3 |
| 1743 | Seq. ID No. 3611 | Seq. ID No. 3612 | YES | IM | 1 |
| 1745 | Seq. ID No. 3617 | Seq. ID No. 3618 | yes | Peri | 0 |
| 1747 | Seq. ID No. 3619 | Seq. ID No. 3620 | YES | IM | 2 |
| 1749a | Seq. ID No. 3621 | Seq. ID No. 3622 | yes | IM | 3 |
| 1750a | Seq. ID No. 3625 | Seq. ID No. 3626 | maybe | IM | 2 |
| 1752b | Seq. ID No. 3629 | Seq. ID No. 3630 | yes | Peri | 1 |
| 1756b | Seq. ID No. 3635 | Seq. ID No. 3636 | maybe | OM | 1 |
| 1778a | Seq. ID No. 3677 | Seq. ID No. 3678 | maybe | IM | 1 |
| 1778b | Seq. ID No. 3679 | Seq. ID No. 3680 | maybe | Cyto | 1 |
| 1784b | Seq. ID No. 3689 | Seq. ID No. 3690 | YES | IM | 1 |
| 1794a | Seq. ID No. 3717 | Seq. ID No. 3718 | maybe | OM | 1 |
| 1798a | Seq. ID No. 3723 | Seq. ID No. 3724 | yes | IM | 2 |
| 1798c | Seq. ID No. 3727 | Seq. ID No. 3728 | maybe | IM | 4 |
| 1805 | Seq. ID No. 3741 | Seq. ID No. 3742 | yes | IM | 3 |
| 1806b | Seq. ID No. 3745 | Seq. ID No. 3746 | yes | IM | 5 |
| 1808a | Seq. ID No. 3753 | Seq. ID No. 3754 | YES | IM | 3 |
| 1808b | Seq. ID No. 3755 | Seq. ID No. 3756 | YES | IM | 5 |
| 1808c | Seq. ID No. 3757 | Seq. ID No. 3758 | maybe | IM | 9 |
| 1809a | Seq. ID No. 3761 | Seq. ID No. 3762 | maybe | IM | 5 |
| 1809b | Seq. ID No. 3763 | Seq. ID No. 3764 | yes | IM | 7 |
| 1809c | Seq. ID No. 3765 | Seq. ID No. 3766 | YES | IM | 8 |
| 1812a | Seq. ID No. 3781 | Seq. ID No. 3782 | YES | IM | 1 |
| 1812b | Seq. ID No. 3783 | Seq. ID No. 3784 | YES | IM | 2 |
| 1816a | Seq. ID No. 3793 | Seq. ID No. 3794 | yes | Peri | 1 |
| 1817a | Seq. ID No. 3799 | Seq. ID No. 3800 | maybe | IM | 3 |
| 1822 | Seq. ID No. 3807 | Seq. ID No. 3808 | maybe | Cyto | 1 |
| 1825 | Seq. ID No. 3813 | Seq. ID No. 3814 | yes | IM | 2 |
| 1826a | Seq. ID No. 3815 | Seq. ID No. 3816 | NO | Cyto | 0 |
| 1826b | Seq. ID No. 3817 | Seq. ID No. 3818 | NO | Cyto | 0 |
| 1838a | Seq. ID No. 3837 | Seq. ID No. 3838 | yes | IM | 3 |
| 1838b | Seq. ID No. 3839 | Seq. ID No. 3840 | yes | IM | 4 |
| 1842a | Seq. ID No. 3849 | Seq. ID No. 3850 | maybe | IM | 2 |
| 1842b | Seq. ID No. 3851 | Seq. ID No. 3852 | YES | IM | 6 |
| 1842c | Seq. ID No. 3853 | Seq. ID No. 3854 | maybe | IM | 8 |
| 1845a | Seq. ID No. 3859 | Seq. ID No. 3860 | maybe | IM | 3 |
| 1845b | Seq. ID No. 3861 | Seq. ID No. 3862 | yes | IM | 8 |
| 1845c | Seq. ID No. 3863 | Seq. ID No. 3864 | maybe | IM | 10 |
| 1848a | Seq. ID No. 3865 | Seq. ID No. 3866 | yes | IM | 7 |
| 1854a | Seq. ID No. 3879 | Seq. ID No. 3880 | NO | IM | 3 |
| 1857b | Seq. ID No. 3889 | Seq. ID No. 3890 | yes | IM | 5 |
| 1857c | Seq. ID No. 3891 | Seq. ID No. 3892 | yes | IM | 6 |
| 1858 | Seq. ID No. 3895 | Seq. ID No. 3896 | yes | Cyto | 0 |
| 1862a | Seq. ID No. 3903 | Seq. ID No. 3904 | YES | IM | 1 |
| 1868 | Seq. ID No. 3915 | Seq. ID No. 3916 | yes | IM | 1 |
| 1873b | Seq. ID No. 3923 | Seq. ID No. 3924 | yes | Peri | 1 |
| 1873c | Seq. ID No. 3925 | Seq. ID No. 3926 | yes | Peri | 1 |
| 1894c | Seq. ID No. 3959 | Seq. ID No. 3960 | maybe | IM | 1 |
| 1896a | Seq. ID No. 3963 | Seq. ID No. 3964 | maybe | Peri | 1 |
| 1898a | Seq. ID No. 3973 | Seq. ID No. 3974 | maybe | IM | 2 |
| 1898b | Seq. ID No. 3975 | Seq. ID No. 3976 | yes | IM | 4 |
| 1916 | Seq. ID No. 3993 | Seq. ID No. 3994 | maybe | IM | 1 |
| 1917a | Seq. ID No. 3995 | Seq. ID No. 3996 | yes | IM | 0 |
| 1917b | Seq. ID No. 3997 | Seq. ID No. 3998 | yes | IM | 0 |
| 1919a | Seq. ID No. 4005 | Seq. ID No. 4006 | YES | Peri | 1 |
| 1919b | Seq. ID No. 4007 | Seq. ID No. 4008 | YES | IM | 1 |
| 1919c | Seq. ID No. 4009 | Seq. ID No. 4010 | yes | IM | 1 |
| 1931a | Seq. ID No. 4025 | Seq. ID No. 4026 | NO | IM | 2 |
| 1931b | Seq. ID No. 4027 | Seq. ID No. 4028 | YES | IM | 3 |
| 1936 | Seq. ID No. 4039 | Seq. ID No. 4040 | yes | IM | 6 |
| 1937a | Seq. ID No. 4041 | Seq. ID No. 4042 | yes | IM | 2 |
| 1937b | Seq. ID No. 4043 | Seq. ID No. 4044 | maybe | IM | 3 |
| 1937c | Seq. ID No. 4045 | Seq. ID No. 4046 | maybe | IM | 4 |
| 1937d | Seq. ID No. 4047 | Seq. ID No. 4048 | maybe | IM | 7 |
| 1937e | Seq. ID No. 4049 | Seq. ID No. 4050 | maybe | IM | 7 |
| 1941b | Seq. ID No. 4061 | Seq. ID No. 4062 | yes | IM | 4 |
| 1946a | Seq. ID No. 4071 | Seq. ID No. 4072 | NO | IM | 4 |
| 1946b | Seq. ID No. 4073 | Seq. ID No. 4074 | YES | IM | 8 |
| 1950 | Seq. ID No. 4079 | Seq. ID No. 4080 | yes | OM | 1 |
| 1951a | Seq. ID No. 4081 | Seq. ID No. 4082 | yes | IM | 3 |
| 1951b | Seq. ID No. 4083 | Seq. ID No. 4084 | maybe | IM | 4 |
| 1958a | Seq. ID No. 4101 | Seq. ID No. 4102 | maybe | IM | 4 |
| 1958b | Seq. ID No. 4103 | Seq. ID No. 4104 | maybe | IM | 7 |
| 1960c | Seq. ID No. 4111 | Seq. ID No. 4112 | yes | IM | 1 |
| 1970b | Seq. ID No. 4133 | Seq. ID No. 4134 | maybe | OM | 1 |
| 1972 | Seq. ID No. 4135 | Seq. ID No. 4136 | yes | Cyto | 1 |
| 1973 | Seq. ID No. 4137 | Seq. ID No. 4138 | YES | OM | 1 |
| 1976 | Seq. ID No. 4139 | Seq. ID No. 4140 | yes | IM | 1 |
| 1977a | Seq. ID No. 4141 | Seq. ID No. 4142 | YES | OM | 1 |
| 1977b | Seq. ID No. 4143 | Seq. ID No. 4144 | YES | OM | 1 |
| 1977c | Seq. ID No. 4145 | Seq. ID No. 4146 | YES | OM | 1 |
| 1980a | Seq. ID No. 4151 | Seq. ID No. 4152 | NO | Cyto | 1 |
| 1980b | Seq. ID No. 4153 | Seq. ID No. 4154 | maybe | Cyto | 1 |
| 1991 | Seq. ID No. 4181 | Seq. ID No. 4182 | yes | Cyto | 1 |
| 2016a | Seq. ID No. 4221 | Seq. ID No. 4222 | yes | IM | 2 |
| 2016b | Seq. ID No. 4223 | Seq. ID No. 4224 | maybe | IM | 4 |
| 2016c | Seq. ID No. 4225 | Seq. ID No. 4226 | YES | IM | 7 |
| 2016d | Seq. ID No. 4227 | Seq. ID No. 4228 | maybe | IM | 11 |
| 2016f | Seq. ID No. 4231 | Seq. ID No. 4232 | maybe | IM | 13 |
| 2016g | Seq. ID No. 4233 | Seq. ID No. 4234 | NO | IM | 13 |
| 2023a | Seq. ID No. 4241 | Seq. ID No. 4242 | maybe | IM | 4 |
| 2024b | Seq. ID No. 4247 | Seq. ID No. 4248 | yes | IM | 2 |
| 2025 | Seq. ID No. 4253 | Seq. ID No. 4254 | yes | OM | 1 |
| 2030a | Seq. ID No. 4257 | Seq. ID No. 4258 | yes | IM | 4 |
| 2030b | Seq. ID No. 4259 | Seq. ID No. 4260 | yes | IM | 5 |
| 2030c | Seq. ID No. 4261 | Seq. ID No. 4262 | yes | IM | 5 |
| 2030d | Seq. ID No. 4263 | Seq. ID No. 4264 | yes | IM | 5 |
| 2032c | Seq. ID No. 4265 | Seq. ID No. 4266 | maybe | Cyto | |
| 2035b | Seq. ID No. 4269 | Seq. ID No. 4270 | yes | Cyto | 0 |
| 2038a | Seq. ID No. 4275 | Seq. ID No. 4276 | NO | Cyto | 0 |
| 2045a | Seq. ID No. 4285 | Seq. ID No. 4286 | maybe | Cyto | 1 |
| 2045b | Seq. ID No. 4287 | Seq. ID No. 4288 | maybe | Cyto | 1 |
| 2049c | Seq. ID No. 4297 | Seq. ID No. 4298 | maybe | Cyto | |
| 2056 | Seq. ID No. 4307 | Seq. ID No. 4308 | maybe | Peri | 1 |
| 2059a | Seq. ID No. 4311 | Seq. ID No. 4312 | yes | IM | 2 |
| 2059b | Seq. ID No. 4313 | Seq. ID No. 4314 | maybe | IM | 4 |
| 2074b | Seq. ID No. 4343 | Seq. ID No. 4344 | YES | OM | 1 |
| 2092 | Seq. ID No. 4379 | Seq. ID No. 4380 | yes | IM | 2 |
| 2096a | Seq. ID No. 4385 | Seq. ID No. 4386 | YES | IM | 4 |
| 2096c | Seq. ID No. 4389 | Seq. ID No. 4390 | yes | IM | 10 |
| 2096d | Seq. ID No. 4391 | Seq. ID No. 4392 | yes | IM | 10 |
| 2101 | Seq. ID No. 4399 | Seq. ID No. 4400 | YES | Peri | 1 |
| 2105 | Seq. ID No. 4403 | Seq. ID No. 4404 | maybe | Cyto | 1 |
| 2120a | Seq. ID No. 4437 | Seq. ID No. 4438 | NO | IM | 1 |
| 2120b | Seq. ID No. 4439 | Seq. ID No. 4440 | NO | IM | 1 |
| 2122b | Seq. ID No. 4441 | Seq. ID No. 4442 | maybe | IM | 4 |
| 2140a | Seq. ID No. 4469 | Seq. ID No. 4470 | YES | IM | 4 |
| 2142a | Seq. ID No. 4473 | Seq. ID No. 4474 | yes | IM | 1 |
| 2146 | Seq. ID No. 4481 | Seq. ID No. 4482 | yes | OM | 1 |
| 2147a | Seq. ID No. 4483 | Seq. ID No. 4484 | YES | IM | 2 |
| 2147b | Seq. ID No. 4485 | Seq. ID No. 4486 | maybe | IM | 5 |
| 2150a | Seq. ID No. 4493 | Seq. ID No. 4494 | YES | IM | 1 |
| 2150b | Seq. ID No. 4495 | Seq. ID No. 4496 | YES | IM | 2 |
| 2150c | Seq. ID No. 4497 | Seq. ID No. 4498 | maybe | IM | 2 |
| 2159a | Seq. ID No. 4513 | Seq. ID No. 4514 | maybe | IM | 2 |
| 2159b | Seq. ID No. 4515 | Seq. ID No. 4516 | maybe | IM | 2 |
| 2161b | Seq. ID No. 4527 | Seq. ID No. 4528 | maybe | Cyto | 1 |
| 2161c | Seq. ID No. 4529 | Seq. ID No. 4530 | maybe | Cyto | 1 |
| 2165 | Seq. ID No. 4533 | Seq. ID No. 4534 | YES | IM | 1 |
| 2190a | Seq. ID No. 4587 | Seq. ID No. 4588 | maybe | IM | 1 |
| 2190b | Seq. ID No. 4589 | Seq. ID No. 4590 | NO | Peri | 1 |
| 2219a | Seq. ID No. 4639 | Seq. ID No. 4640 | yes | IM | 1 |
| 2219b | Seq. ID No. 4641 | Seq. ID No. 4642 | maybe | IM | 4 |
| 2219c | Seq. ID No. 4643 | Seq. ID No. 4644 | maybe | IM | 4 |
| 2225 | Seq. ID No. 4659 | Seq. ID No. 4660 | yes | OM | 0 |
| 2236a | Seq. ID No. 4681 | Seq. ID No. 4682 | yes | IM | 2 |
| 2239b | Seq. ID No. 4687 | Seq. ID No. 4688 | maybe | Cyto | 0 |
| 2243a | Seq. ID No. 4695 | Seq. ID No. 4696 | maybe | IM | 2 |
| 2249 | Seq. ID No. 4705 | Seq. ID No. 4706 | yes | OM | 1 |
| 2253a | Seq. ID No. 4707 | Seq. ID No. 4708 | NO | IM | 2 |
| 2253b | Seq. ID No. 4709 | Seq. ID No. 4710 | YES | IM | 4 |
| 2253c | Seq. ID No. 4711 | Seq. ID No. 4712 | YES | IM | 5 |
| 2255a | Seq. ID No. 4715 | Seq. ID No. 4716 | NO | IM | 3 |
| 2255b | Seq. ID No. 4717 | Seq. ID No. 4718 | yes | IM | 6 |
| 2264b | Seq. ID No. 4737 | Seq. ID No. 4738 | yes | IM | 1 |
| 2276a | Seq. ID No. 4759 | Seq. ID No. 4760 | NO | IM | 1 |
| 2300 | Seq. ID No. 4813 | Seq. ID No. 4814 | yes | IM | 4 |
| 2308 | Seq. ID No. 4827 | Seq. ID No. 4828 | YES | OM | 1 |
| 2320 | Seq. ID No. 4853 | Seq. ID No. 4854 | maybe | IM | 1 |
| 2324 | Seq. ID No. 4863 | Seq. ID No. 4864 | maybe | IM | 2 |
| 2329a | Seq. ID No. 4869 | Seq. ID No. 4870 | yes | IM | 1 |
| 2329b | Seq. ID No. 4871 | Seq. ID No. 4872 | YES | IM | 3 |
| 2329c | Seq. ID No. 4873 | Seq. ID No. 4874 | YES | IM | 6 |
| 2329d | Seq. ID No. 4875 | Seq. ID No. 4876 | yes | IM | 7 |
| 2357 | Seq. ID No. 4919 | Seq. ID No. 4920 | yes | IM | 2 |
| 2358 | Seq. ID No. 4921 | Seq. ID No. 4922 | YES | IM | 2 |
| 2360 | Seq. ID No. 4923 | Seq. ID No. 4924 | NO | Cyto | 1 |
| 2363a | Seq. ID No. 4929 | Seq. ID No. 4930 | yes | IM | 1 |
| 2363b | Seq. ID No. 4931 | Seq. ID No. 4932 | yes | IM | 1 |
| 2371a | Seq. ID No. 4949 | Seq. ID No. 4950 | NO | IM | 1 |
| 2372 | Seq. ID No. 4951 | Seq. ID No. 4952 | maybe | Peri | 1 |
| 2382c | Seq. ID No. 4975 | Seq. ID No. 4976 | maybe | Cyto | |
| 2390 | Seq. ID No. 4987 | Seq. ID No. 4988 | NO | Peri | 1 |
| 2397 | Seq. ID No. 4999 | Seq. ID No. 5000 | maybe | Peri | 0 |
| 2402b | Seq. ID No. 5013 | Seq. ID No. 5014 | maybe | IM | 1 |
| 2412 | Seq. ID No. 5031 | Seq. ID No. 5032 | yes | IM | 2 |
| 2423a | Seq. ID No. 5061 | Seq. ID No. 5062 | yes | IM | 5 |
| 2423b | Seq. ID No. 5063 | Seq. ID No. 5064 | yes | IM | 5 |
| 2424c | Seq. ID No. 5069 | Seq. ID No. 5070 | yes | IM | 1 |
| 2434 | Seq. ID No. 5085 | Seq. ID No. 5086 | maybe | Cyto | 1 |
| 2445 | Seq. ID No. 5103 | Seq. ID No. 5104 | yes | IM | 1 |
| 2453 | Seq. ID No. 5115 | Seq. ID No. 5116 | YES | OM | 0 |
| 2456c | Seq. ID No. 5123 | Seq. ID No. 5124 | yes | IM | 8 |
| 2456d | Seq. ID No. 5125 | Seq. ID No. 5126 | maybe | IM | 9 |
| 2458a | Seq. ID No. 5129 | Seq. ID No. 5130 | maybe | IM | 5 |
| 2458b | Seq. ID No. 5131 | Seq. ID No. 5132 | yes | IM | 6 |
| 2458c | Seq. ID No. 5133 | Seq. ID No. 5134 | yes | IM | 7 |
| 2458d | Seq. ID No. 5135 | Seq. ID No. 5136 | yes | IM | 7 |
| 2458e | Seq. ID No. 5137 | Seq. ID No. 5138 | yes | IM | 8 |
| 2458f | Seq. ID No. 5139 | Seq. ID No. 5140 | yes | IM | 9 |
| 2458g | Seq. ID No. 5141 | Seq. ID No. 5142 | yes | IM | 9 |
| 2468a | Seq. ID No. 5159 | Seq. ID No. 5160 | yes | IM | 1 |
| 2469b | Seq. ID No. 5165 | Seq. ID No. 5166 | yes | IM | 2 |
| 2474b | Seq. ID No. 5175 | Seq. ID No. 5176 | yes | IM | 3 |
| 2484a | Seq. ID No. 5197 | Seq. ID No. 5198 | maybe | Peri | 1 |
| 2484b | Seq. ID No. 5199 | Seq. ID No. 5200 | maybe | Peri | 1 |
| 2490 | Seq. ID No. 5205 | Seq. ID No. 5206 | yes | IM | 1 |
| 2492a | Seq. ID No. 5207 | Seq. ID No. 5208 | yes | IM | 1 |
| 2495a | Seq. ID No. 5213 | Seq. ID No. 5214 | maybe | IM | 9 |
| 2497b | Seq. ID No. 5219 | Seq. ID No. 5220 | yes | Peri | 2 |
| 2500a | Seq. ID No. 5223 | Seq. ID No. 5224 | yes | OM | 1 |
| 2518a | Seq. ID No. 5267 | Seq. ID No. 5268 | YES | Cyto | 1 |
| 2520a | Seq. ID No. 5273 | Seq. ID No. 5274 | NO | Cyto | 0 |
| 2520b | Seq. ID No. 5275 | Seq. ID No. 5276 | NO | Cyto | 0 |
| 2520c | Seq. ID No. 5277 | Seq. ID No. 5278 | NO | Cyto | 0 |
| 2520d | Seq. ID No. 5279 | Seq. ID No. 5280 | NO | Cyto | 0 |
| 2521 | Seq. ID No. 5281 | Seq. ID No. 5282 | maybe | IM | 3 |
| 2525a | Seq. ID No. 5285 | Seq. ID No. 5286 | yes | IM | 1 |
| 2546 | Seq. ID No. 5327 | Seq. ID No. 5328 | NO | Cyto | 1 |
| 2547b | Seq. ID No. 5329 | Seq. ID No. 5330 | yes | IM | 1 |
| 2547c | Seq. ID No. 5331 | Seq. ID No. 5332 | yes | IM | 1 |
| 2550c | Seq. ID No. 5341 | Seq. ID No. 5342 | YES | IM | |
| 2564b | Seq. ID No. 5377 | Seq. ID No. 5378 | yes | OM | 1 |
| 2564c | Seq. ID No. 5379 | Seq. ID No. 5380 | yes | OM | 1 |
| 2567a | Seq. ID No. 5385 | Seq. ID No. 5386 | yes | IM | 4 |
| 2567b | Seq. ID No. 5387 | Seq. ID No. 5388 | YES | IM | 5 |
| 2582a | Seq. ID No. 5405 | Seq. ID No. 5406 | yes | IM | 4 |
| 2587a | Seq. ID No. 5413 | Seq. ID No. 5414 | NO | Peri | 2 |
| 2587b | Seq. ID No. 5415 | Seq. ID No. 5416 | maybe | IM | 5 |
| 2596 | Seq. ID No. 5433 | Seq. ID No. 5434 | NO | IM | 1 |
| 2610 | Seq. ID No. 5453 | Seq. ID No. 5454 | yes | IM | 1 |
| 2616a | Seq. ID No. 5459 | Seq. ID No. 5460 | yes | IM | 2 |
| 2616b | Seq. ID No. 5461 | Seq. ID No. 5462 | yes | IM | 3 |
| 2616c | Seq. ID No. 5463 | Seq. ID No. 5464 | yes | IM | 4 |
| 2616d | Seq. ID No. 5465 | Seq. ID No. 5466 | YES | IM | 5 |
| 2623b | Seq. ID No. 5491 | Seq. ID No. 5492 | maybe | OM | 1 |
| 2625a | Seq. ID No. 5497 | Seq. ID No. 5498 | YES | Peri | 1 |
| 2628 | Seq. ID No. 5509 | Seq. ID No. 5510 | yes | IM | 1 |
| 2629b | Seq. ID No. 5513 | Seq. ID No. 5514 | YES | OM | 1 |
| 2631 | Seq. ID No. 5515 | Seq. ID No. 5516 | NO | Cyto | 1 |
| 2632 | Seq. ID No. 5517 | Seq. ID No. 5518 | NO | Cyto | 1 |
| 2633d | Seq. ID No. 5525 | Seq. ID No. 5526 | maybe | OM | 1 |
| 2637a | Seq. ID No. 5529 | Seq. ID No. 5530 | yes | OM | 1 |
| 2637b | Seq. ID No. 5531 | Seq. ID No. 5532 | yes | IM | 1 |
| 2642 | Seq. ID No. 5533 | Seq. ID No. 5534 | NO | IM | 2 |
| 2648a | Seq. ID No. 5543 | Seq. ID No. 5544 | YES | IM | 0 |
| 2648b | Seq. ID No. 5545 | Seq. ID No. 5546 | YES | IM | 0 |
| 2660 | Seq. ID No. 5569 | Seq. ID No. 5570 | yes | Peri | 1 |
| 2669a | Seq. ID No. 5587 | Seq. ID No. 5588 | YES | Peri | 2 |
| 2669b | Seq. ID No. 5589 | Seq. ID No. 5590 | YES | IM | 2 |
| 2669c | Seq. ID No. 5591 | Seq. ID No. 5592 | YES | IM | 2 |
| 2672 | Seq. ID No. 5593 | Seq. ID No. 5594 | NO | IM | 3 |
| 2673 | Seq. ID No. 5595 | Seq. ID No. 5596 | maybe | IM | 2 |
| 2674a | Seq. ID No. 5597 | Seq. ID No. 5598 | maybe | IM | 4 |
| 2679a | Seq. ID No. 5609 | Seq. ID No. 5610 | NO | IM | 4 |
| 2679b | Seq. ID No. 5611 | Seq. ID No. 5612 | YES | IM | 5 |
| 2680c | Seq. ID No. 5621 | Seq. ID No. 5622 | YES | IM | 1 |
| 2683a | Seq. ID No. 5623 | Seq. ID No. 5624 | yes | IM | 1 |
| 2683b | Seq. ID No. 5625 | Seq. ID No. 5626 | yes | IM | 1 |
| 2688a | Seq. ID No. 5633 | Seq. ID No. 5634 | maybe | IM | 2 |
| 2688c | Seq. ID No. 5637 | Seq. ID No. 5638 | maybe | IM | 4 |
| 2689a | Seq. ID No. 5639 | Seq. ID No. 5640 | maybe | Peri | 0 |
| 2689b | Seq. ID No. 5641 | Seq. ID No. 5642 | YES | Peri | 0 |
| 2690a | Seq. ID No. 5643 | Seq. ID No. 5644 | yes | Peri | 1 |
| 2692b | Seq. ID No. 5649 | Seq. ID No. 5650 | maybe | IM | 1 |
| 2696a | Seq. ID No. 5659 | Seq. ID No. 5660 | yes | IM | 8 |
| 2710c | Seq. ID No. 5667 | Seq. ID No. 5668 | maybe | Cyto | |
| 2718a | Seq. ID No. 5681 | Seq. ID No. 5682 | YES | IM | 3 |
| 2718b | Seq. ID No. 5683 | Seq. ID No. 5684 | yes | IM | 3 |
| 2718c | Seq. ID No. 5685 | Seq. ID No. 5686 | yes | IM | 4 |
| 2719a | Seq. ID No. 5687 | Seq. ID No. 5688 | maybe | Cyto | 0 |
| 2719b | Seq. ID No. 5689 | Seq. ID No. 5690 | maybe | Cyto | 0 |
| 2723 | Seq. ID No. 5699 | Seq. ID No. 5700 | maybe | IM | 4 |
| 2725a | Seq. ID No. 5703 | Seq. ID No. 5704 | maybe | Peri | 2 |
| 2725b | Seq. ID No. 5705 | Seq. ID No. 5706 | maybe | Cyto | 2 |
| 2728a | Seq. ID No. 5709 | Seq. ID No. 5710 | yes | IM | 1 |
| 2728b | Seq. ID No. 5711 | Seq. ID No. 5712 | yes | IM | 1 |
| 2734a | Seq. ID No. 5719 | Seq. ID No. 5720 | YES | IM | 3 |
| 2738a | Seq. ID No. 5729 | Seq. ID No. 5730 | maybe | IM | 10 |
| 2738c | Seq. ID No. 5733 | Seq. ID No. 5734 | yes | IM | 12 |
| 2738d | Seq. ID No. 5735 | Seq. ID No. 5736 | maybe | IM | 12 |
| 2742b | Seq. ID No. 5739 | Seq. ID No. 5740 | yes | Peri | |
| 2749a | Seq. ID No. 5763 | Seq. ID No. 5764 | NO | IM | 3 |
| 2749b | Seq. ID No. 5765 | Seq. ID No. 5766 | yes | IM | 4 |
| 2749c | Seq. ID No. 5767 | Seq. ID No. 5768 | NO | IM | 8 |
| 2749e | Seq. ID No. 5771 | Seq. ID No. 5772 | YES | IM | 10 |
| 2757b | Seq. ID No. 5783 | Seq. ID No. 5784 | YES | IM | 5 |
| 2758a | Seq. ID No. 5785 | Seq. ID No. 5786 | NO | Cyto | 0 |
| 2758d | Seq. ID No. 5791 | Seq. ID No. 5792 | maybe | Cyto | 1 |
| 2760a | Seq. ID No. 5797 | Seq. ID No. 5798 | yes | Peri | 1 |
| 2770 | Seq. ID No. 5829 | Seq. ID No. 5830 | maybe | IM | 1 |
| 2791 | Seq. ID No. 5879 | Seq. ID No. 5880 | maybe | IM | 1 |
| 2794 | Seq. ID No. 5883 | Seq. ID No. 5884 | NO | IM | 2 |
| 2796a | Seq. ID No. 5885 | Seq. ID No. 5886 | maybe | IM | 2 |
| 2799a | Seq. ID No. 5889 | Seq. ID No. 5890 | yes | OM | 1 |
| 2801a | Seq. ID No. 5893 | Seq. ID No. 5894 | yes | IM | 1 |
| 2801b | Seq. ID No. 5895 | Seq. ID No. 5896 | YES | IM | 2 |
| 2805a | Seq. ID No. 5899 | Seq. ID No. 5900 | YES | IM | 2 |
| 2805b | Seq. ID No. 5901 | Seq. ID No. 5902 | YES | IM | 4 |
| 2810a | Seq. ID No. 5905 | Seq. ID No. 5906 | yes | IM | 3 |
| 2810c | Seq. ID No. 5909 | Seq. ID No. 5910 | yes | IM | 4 |
| 2818a | Seq. ID No. 5921 | Seq. ID No. 5922 | maybe | IM | 2 |
| 2818b | Seq. ID No. 5923 | Seq. ID No. 5924 | yes | IM | 5 |
| 2818d | Seq. ID No. 5927 | Seq. ID No. 5928 | YES | IM | 6 |
| 2818e | Seq. ID No. 5929 | Seq. ID No. 5930 | YES | IM | 6 |
| 2822a | Seq. ID No. 5939 | Seq. ID No. 5940 | YES | IM | 3 |
| 2869a | Seq. ID No. 6023 | Seq. ID No. 6024 | maybe | OM | 1 |
| 2869b | Seq. ID No. 6025 | Seq. ID No. 6026 | yes | OM | 1 |
| 2869c | Seq. ID No. 6027 | Seq. ID No. 6028 | yes | IM | 1 |
| 2881 | Seq. ID No. 6043 | Seq. ID No. 6044 | yes | IM | 1 |
| 2883a | Seq. ID No. 6047 | Seq. ID No. 6048 | yes | IM | 6 |
| 2883d | Seq. ID No. 6053 | Seq. ID No. 6054 | yes | IM | 8 |
| 2887a | Seq. ID No. 6059 | Seq. ID No. 6060 | yes | Peri | 1 |
| 2888 | Seq. ID No. 6063 | Seq. ID No. 6064 | maybe | Cyto | 1 |
| 2891a | Seq. ID No. 6067 | Seq. ID No. 6068 | NO | Peri | 1 |
| 2900a | Seq. ID No. 6087 | Seq. ID No. 6088 | YES | Peri | 1 |
| 2900b | Seq. ID No. 6089 | Seq. ID No. 6090 | YES | Peri | 1 |
| 2901a | Seq. ID No. 6091 | Seq. ID No. 6092 | YES | IM | 1 |
| 2902 | Seq. ID No. 6095 | Seq. ID No. 6096 | YES | IM | 0 |
| 2906 | Seq. ID No. 6103 | Seq. ID No. 6104 | YES | IM | 2 |
| 2909b | Seq. ID No. 6107 | Seq. ID No. 6108 | maybe | Cyto | 1 |
| 2909c | Seq. ID No. 6109 | Seq. ID No. 6110 | yes | IM | 4 |
| 2909d | Seq. ID No. 6111 | Seq. ID No. 6112 | yes | IM | 4 |
| 2913a | Seq. ID No. 6115 | Seq. ID No. 6116 | yes | IM | 2 |
| 2913b | Seq. ID No. 6117 | Seq. ID No. 6118 | yes | IM | 2 |
| 2922b | Seq. ID No. 6135 | Seq. ID No. 6136 | yes | Cyto | 0 |
| 2923a | Seq. ID No. 6137 | Seq. ID No. 6138 | yes | Cyto | |
| 2929b | Seq. ID No. 6159 | Seq. ID No. 6160 | maybe | Cyto | |
| 2931a | Seq. ID No. 6161 | Seq. ID No. 6162 | yes | IM | 5 |
| 2931b | Seq. ID No. 6163 | Seq. ID No. 6164 | NO | IM | 6 |
| 2931c | Seq. ID No. 6165 | Seq. ID No. 6166 | yes | IM | 13 |
| 2933b | Seq. ID No. 6169 | Seq. ID No. 6170 | YES | IM | 1 |
| 2941a | Seq. ID No. 6183 | Seq. ID No. 6184 | yes | IM | 2 |
| 2941b | Seq. ID No. 6185 | Seq. ID No. 6186 | YES | IM | 5 |
| 2941c | Seq. ID No. 6187 | Seq. ID No. 6188 | YES | IM | 5 |
| 2943a | Seq. ID No. 6189 | Seq. ID No. 6190 | yes | IM | 3 |
| 2943b | Seq. ID No. 6191 | Seq. ID No. 6192 | yes | IM | 5 |
| 2944b | Seq. ID No. 6197 | Seq. ID No. 6198 | YES | IM | 1 |
| 2948a | Seq. ID No. 6203 | Seq. ID No. 6204 | YES | IM | 1 |
| 2948b | Seq. ID No. 6205 | Seq. ID No. 6206 | YES | IM | 5 |
| 2948d | Seq. ID No. 6209 | Seq. ID No. 6210 | yes | IM | 7 |
| 2952 | Seq. ID No. 6217 | Seq. ID No. 6218 | yes | Cyto | 1 |
| 2955a | Seq. ID No. 6221 | Seq. ID No. 6222 | YES | IM | 4 |
| 2955b | Seq. ID No. 6223 | Seq. ID No. 6224 | YES | IM | 4 |
| 2955c | Seq. ID No. 6225 | Seq. ID No. 6226 | yes | IM | 6 |
| 2955d | Seq. ID No. 6227 | Seq. ID No. 6228 | maybe | IM | 7 |
| 2955e | Seq. ID No. 6229 | Seq. ID No. 6230 | YES | IM | 11 |
| 2957 | Seq. ID No. 6235 | Seq. ID No. 6236 | yes | IM | 4 |
| 2958a | Seq. ID No. 6237 | Seq. ID No. 6238 | maybe | IM | 2 |
| 2958c | Seq. ID No. 6241 | Seq. ID No. 6242 | maybe | IM | 2 |
| 2959 | Seq. ID No. 6243 | Seq. ID No. 6244 | yes | IM | 2 |
| 2960a | Seq. ID No. 6245 | Seq. ID No. 6246 | maybe | IM | 2 |
| 2962b | Seq. ID No. 6249 | Seq. ID No. 6250 | yes | IM | 1 |
| 2965a | Seq. ID No. 6253 | Seq. ID No. 6254 | yes | Cyto | 1 |
| 2966 | Seq. ID No. 6259 | Seq. ID No. 6260 | yes | IM | 1 |
| 2969 | Seq. ID No. 6263 | Seq. ID No. 6264 | maybe | IM | 1 |
| 2970a | Seq. ID No. 6265 | Seq. ID No. 6266 | yes | IM | 1 |
| 2970b | Seq. ID No. 6267 | Seq. ID No. 6268 | yes | IM | 1 |
| 2974a | Seq. ID No. 6273 | Seq. ID No. 6274 | maybe | Peri | 2 |
| 2974b | Seq. ID No. 6275 | Seq. ID No. 6276 | yes | IM | 4 |
| 2978a | Seq. ID No. 6283 | Seq. ID No. 6284 | yes | Peri | 1 |
| 2978b | Seq. ID No. 6285 | Seq. ID No. 6286 | yes | Peri | 1 |
| 2980a | Seq. ID No. 6289 | Seq. ID No. 6290 | maybe | IM | 3 |
| 2986a | Seq. ID No. 6301 | Seq. ID No. 6302 | maybe | IM | 6 |
| 2986b | Seq. ID No. 6303 | Seq. ID No. 6304 | yes | IM | 7 |
| 2993a | Seq. ID No. 6325 | Seq. ID No. 6326 | yes | OM | 0 |
| 2993b | Seq. ID No. 6327 | Seq. ID No. 6328 | yes | OM | 0 |
| 3016a | Seq. ID No. 6363 | Seq. ID No. 6364 | yes | IM | 4 |
| 3016b | Seq. ID No. 6365 | Seq. ID No. 6366 | yes | IM | 7 |
| 3021 | Seq. ID No. 6377 | Seq. ID No. 6378 | NO | Peri | 1 |
| 3023 | Seq. ID No. 6381 | Seq. ID No. 6382 | maybe | IM | 1 |
| 3024a | Seq. ID No. 6383 | Seq. ID No. 6384 | maybe | Peri | 1 |
| 3037a | Seq. ID No. 6415 | Seq. ID No. 6416 | YES | OM | 1 |
| 3042a | Seq. ID No. 6425 | Seq. ID No. 6426 | yes | Cyto | 1 |
| 3042b | Seq. ID No. 6427 | Seq. ID No. 6428 | yes | Cyto | 1 |
| 3043a | Seq. ID No. 6429 | Seq. ID No. 6430 | yes | IM | 2 |
| 3043b | Seq. ID No. 6431 | Seq. ID No. 6432 | yes | IM | 2 |
| 3064b | Seq. ID No. 6473 | Seq. ID No. 6474 | YES | Peri | 1 |
| 3066 | Seq. ID No. 6479 | Seq. ID No. 6480 | yes | Cyto | 0 |
| 3076 | Seq. ID No. 6497 | Seq. ID No. 6498 | maybe | IM | 3 |
| 3085 | Seq. ID No. 6517 | Seq. ID No. 6518 | maybe | Cyto | 0 |
| 3096a | Seq. ID No. 6541 | Seq. ID No. 6542 | yes | IM | 1 |
| 3096b | Seq. ID No. 6543 | Seq. ID No. 6544 | YES | IM | 1 |
| 3100a | Seq. ID No. 6555 | Seq. ID No. 6556 | maybe | Cyto | 0 |
| 3100b | Seq. ID No. 6557 | Seq. ID No. 6558 | maybe | Cyto | 0 |
| 3107a | Seq. ID No. 6571 | Seq. ID No. 6572 | yes | OM | 1 |
| 3107b | Seq. ID No. 6573 | Seq. ID No. 6574 | yes | OM | 2 |
| 3110a | Seq. ID No. 6581 | Seq. ID No. 6582 | NO | IM | 1 |
| 3119a | Seq. ID No. 6593 | Seq. ID No. 6594 | NO | IM | 3 |
| 3119b | Seq. ID No. 6595 | Seq. ID No. 6596 | YES | IM | 4 |
| 3119c | Seq. ID No. 6597 | Seq. ID No. 6598 | YES | IM | 4 |
| 3119d | Seq. ID No. 6599 | Seq. ID No. 6600 | YES | IM | 4 |
| 3119e | Seq. ID No. 6601 | Seq. ID No. 6602 | YES | IM | 4 |
| 3119f | Seq. ID No. 6603 | Seq. ID No. 6604 | YES | IM | 4 |
| 3140 | Seq. ID No. 6631 | Seq. ID No. 6632 | maybe | IM | 2 |
| 3145b | Seq. ID No. 6637 | Seq. ID No. 6638 | maybe | OM | 1 |
| 3145c | Seq. ID No. 6639 | Seq. ID No. 6640 | yes | OM | 1 |
| 3147b | Seq. ID No. 6643 | Seq. ID No. 6644 | yes | IM | 0 |
| 3147c | Seq. ID No. 6645 | Seq. ID No. 6646 | yes | OM | 0 |
| 3150a | Seq. ID No. 6649 | Seq. ID No. 6650 | NO | Cyto | 0 |

Listed in Table 6 are 2388 ORFs that meet the criteria of being a non-secretory protein, based on the HMM SignalP program. Of these ORFs, the PSORT program predicted 756 ORFs to be localized to the cytoplasmic inner membrane (IM); 35 ORFs to be localized to the periplasm (Peri); 10 ORFs to be localized to the outer membrane (OM); and 1586 ORFs localized to the cytoplasm. Shown for all ORFs is the number of transmembrane domains predicted by the TopPred2 software (listed in the MSD column).

TABLE 6
SIGNALP: NON-SECRETORY

| ORF | DNA SEQ | Protein SEQ | | |
|---|---|---|---|---|
| Number | ID Number | ID Number | PSORT | MSD |
| 1 | Seq. ID No. 1 | Seq. ID No. 2 | Cyto | 2 |
| 2b | Seq. ID No. 5 | Seq. ID No. 6 | Cyto | 3 |
| 4c | Seq. ID No. 11 | Seq. ID No. 12 | IM | 12 |
| 5 | Seq. ID No. 13 | Seq. ID No. 14 | Cyto | 0 |
| 7 | Seq. ID No. 15 | Seq. ID No. 16 | Cyto | |
| 10 | Seq. ID No. 17 | Seq. ID No. 18 | Cyto | |
| 16 | Seq. ID No. 23 | Seq. ID No. 24 | IM | 0 |
| 17 | Seq. ID No. 25 | Seq. ID No. 26 | Cyto | |
| 18 | Seq. ID No. 27 | Seq. ID No. 28 | Cyto | 1 |
| 20 | Seq. ID No. 29 | Seq. ID No. 30 | IM | 1 |
| 23b | Seq. ID No. 35 | Seq. ID No. 36 | IM | 1 |
| 24 | Seq. ID No. 37 | Seq. ID No. 38 | IM | 1 |
| 32 | Seq. ID No. 39 | Seq. ID No. 40 | Cyto | |
| 36 | Seq. ID No. 41 | Seq. ID No. 42 | IM | 0 |
| 37 | Seq. ID No. 43 | Seq. ID No. 44 | Cyto | |
| 39b | Seq. ID No. 51 | Seq. ID No. 52 | IM | 5 |
| 41a | Seq. ID No. 55 | Seq. ID No. 56 | Cyto | |
| 41b | Seq. ID No. 57 | Seq. ID No. 58 | Cyto | |
| 41c | Seq. ID No. 59 | Seq. ID No. 60 | IM | 1 |
| 43b | Seq. ID No. 61 | Seq. ID No. 62 | Cyto | |
| 43c | Seq. ID No. 63 | Seq. ID No. 64 | Cyto | |
| 45 | Seq. ID No. 69 | Seq. ID No. 70 | Cyto | |
| 46a | Seq. ID No. 71 | Seq. ID No. 72 | Cyto | |
| 46b | Seq. ID No. 73 | Seq. ID No. 74 | Cyto | |
| 47 | Seq. ID No. 75 | Seq. ID No. 76 | Cyto | |
| 48a | Seq. ID No. 77 | Seq. ID No. 78 | IM | 0 |
| 48b | Seq. ID No. 79 | Seq. ID No. 80 | IM | 0 |
| 48c | Seq. ID No. 81 | Seq. ID No. 82 | IM | 0 |
| 50 | Seq. ID No. 83 | Seq. ID No. 84 | Cyto | 0 |
| 51c | Seq. ID No. 89 | Seq. ID No. 90 | IM | 10 |
| 55 | Seq. ID No. 91 | Seq. ID No. 92 | IM | 1 |
| 56b | Seq. ID No. 93 | Seq. ID No. 94 | Cyto | |
| 57a | Seq. ID No. 95 | Seq. ID No. 96 | IM | 0 |
| 57b | Seq. ID No. 97 | Seq. ID No. 98 | IM | 0 |
| 58 | Seq. ID No. 99 | Seq. ID No. 100 | Cyto | |
| 60a | Seq. ID No. 101 | Seq. ID No. 102 | Cyto | |
| 60b | Seq. ID No. 103 | Seq. ID No. 104 | Cyto | |
| 62a | Seq. ID No. 107 | Seq. ID No. 108 | Cyto | 0 |
| 62b | Seq. ID No. 109 | Seq. ID No. 110 | Cyto | 0 |
| 63a | Seq. ID No. 113 | Seq. ID No. 114 | OM | 0 |
| 63b | Seq. ID No. 115 | Seq. ID No. 116 | OM | 1 |
| 65a | Seq. ID No. 117 | Seq. ID No. 118 | Cyto | |
| 65b | Seq. ID No. 119 | Seq. ID No. 120 | Cyto | |
| 65c | Seq. ID No. 121 | Seq. ID No. 122 | Cyto | |
| 67 | Seq. ID No. 125 | Seq. ID No. 126 | Cyto | |
| 69 | Seq. ID No. 127 | Seq. ID No. 128 | Cyto | 0 |
| 71 | Seq. ID No. 129 | Seq. ID No. 130 | Cyto | 0 |
| 74 | Seq. ID No. 135 | Seq. ID No. 136 | Cyto | |
| 77 | Seq. ID No. 137 | Seq. ID No. 138 | Cyto | |
| 79 | Seq. ID No. 139 | Seq. ID No. 140 | IM | 2 |
| 80b | Seq. ID No. 143 | Seq. ID No. 144 | IM | 4 |
| 80c | Seq. ID No. 145 | Seq. ID No. 146 | IM | 5 |
| 81a | Seq. ID No. 147 | Seq. ID No. 148 | Cyto | |
| 81b | Seq. ID No. 149 | Seq. ID No. 150 | Cyto | |
| 83 | Seq. ID No. 151 | Seq. ID No. 152 | IM | 2 |
| 85a | Seq. ID No. 153 | Seq. ID No. 154 | Cyto | |
| 85b | Seq. ID No. 155 | Seq. ID No. 156 | Cyto | |
| 85c | Seq. ID No. 157 | Seq. ID No. 158 | Cyto | |
| 87c | Seq. ID No. 165 | Seq. ID No. 166 | IM | 2 |
| 88 | Seq. ID No. 167 | Seq. ID No. 168 | IM | 5 |
| 89 | Seq. ID No. 169 | Seq. ID No. 170 | IM | 2 |
| 91b | Seq. ID No. 173 | Seq. ID No. 174 | IM | 0 |
| 91c | Seq. ID No. 175 | Seq. ID No. 176 | IM | 0 |
| 91d | Seq. ID No. 177 | Seq. ID No. 178 | IM | 0 |
| 93a | Seq. ID No. 179 | Seq. ID No. 180 | Cyto | 1 |
| 93b | Seq. ID No. 181 | Seq. ID No. 182 | Peri | 1 |
| 93c | Seq. ID No. 183 | Seq. ID No. 184 | Peri | 2 |
| 94 | Seq. ID No. 185 | Seq. ID No. 186 | IM | 2 |
| 95b | Seq. ID No. 187 | Seq. ID No. 188 | Cyto | |
| 97b | Seq. ID No. 189 | Seq. ID No. 190 | Cyto | |
| 101 | Seq. ID No. 193 | Seq. ID No. 194 | IM | 1 |
| 102 | Seq. ID No. 195 | Seq. ID No. 196 | Cyto | 0 |
| 104a | Seq. ID No. 197 | Seq. ID No. 198 | Cyto | |
| 104b | Seq. ID No. 199 | Seq. ID No. 200 | Cyto | |
| 104c | Seq. ID No. 201 | Seq. ID No. 202 | Peri | 1 |
| 105a | Seq. ID No. 203 | Seq. ID No. 204 | IM | |
| 105b | Seq. ID No. 205 | Seq. ID No. 206 | IM | |
| 106 | Seq. ID No. 207 | Seq. ID No. 208 | Cyto | 0 |
| 111b | Seq. ID No. 219 | Seq. ID No. 220 | IM | 8 |
| 112a | Seq. ID No. 221 | Seq. ID No. 222 | Cyto | 0 |
| 112b | Seq. ID No. 223 | Seq. ID No. 224 | Cyto | 0 |
| 115 | Seq. ID No. 227 | Seq. ID No. 228 | Cyto | |
| 119a | Seq. ID No. 229 | Seq. ID No. 230 | IM | 2 |
| 119b | Seq. ID No. 231 | Seq. ID No. 232 | IM | 3 |
| 121 | Seq. ID No. 233 | Seq. ID No. 234 | Cyto | 0 |
| 124c | Seq. ID No. 239 | Seq. ID No. 240 | IM | 5 |
| 125 | Seq. ID No. 241 | Seq. ID No. 242 | Cyto | |
| 127a | Seq. ID No. 243 | Seq. ID No. 244 | Cyto | |
| 127b | Seq. ID No. 245 | Seq. ID No. 246 | Cyto | |
| 127c | Seq. ID No. 247 | Seq. ID No. 248 | IM | |
| 129c | Seq. ID No. 253 | Seq. ID No. 254 | IM | 10 |
| 134 | Seq. ID No. 261 | Seq. ID No. 262 | Cyto | 1 |
| 135a | Seq. ID No. 263 | Seq. ID No. 264 | Cyto | |
| 135b | Seq. ID No. 265 | Seq. ID No. 266 | Cyto | |
| 137a | Seq. ID No. 267 | Seq. ID No. 268 | Cyto | |
| 137b | Seq. ID No. 269 | Seq. ID No. 270 | Cyto | |
| 142 | Seq. ID No. 275 | Seq. ID No. 276 | Cyto | |
| 144a | Seq. ID No. 279 | Seq. ID No. 280 | Peri | 0 |
| 144b | Seq. ID No. 281 | Seq. ID No. 282 | Peri | 0 |
| 144c | Seq. ID No. 283 | Seq. ID No. 284 | Cyto | 0 |
| 145d | Seq. ID No. 291 | Seq. ID No. 292 | IM | 12 |
| 147 | Seq. ID No. 293 | Seq. ID No. 294 | Cyto | |
| 151a | Seq. ID No. 295 | Seq. ID No. 296 | IM | 1 |
| 151b | Seq. ID No. 297 | Seq. ID No. 298 | IM | 1 |
| 152a | Seq. ID No. 299 | Seq. ID No. 300 | Cyto | 0 |
| 152b | Seq. ID No. 301 | Seq. ID No. 302 | Cyto | 0 |
| 153a | Seq. ID No. 303 | Seq. ID No. 304 | Cyto | |
| 153b | Seq. ID No. 305 | Seq. ID No. 306 | Cyto | |
| 153c | Seq. ID No. 307 | Seq. ID No. 308 | Cyto | |
| 154 | Seq. ID No. 309 | Seq. ID No. 310 | Cyto | 0 |
| 155a | Seq. ID No. 311 | Seq. ID No. 312 | Cyto | |
| 155b | Seq. ID No. 313 | Seq. ID No. 314 | Cyto | |
| 157a | Seq. ID No. 315 | Seq. ID No. 316 | Cyto | |
| 157b | Seq. ID No. 317 | Seq. ID No. 318 | Cyto | |
| 158a | Seq. ID No. 319 | Seq. ID No. 320 | Cyto | |
| 158b | Seq. ID No. 321 | Seq. ID No. 322 | Cyto | |
| 160a | Seq. ID No. 323 | Seq. ID No. 324 | Cyto | |
| 160b | Seq. ID No. 325 | Seq. ID No. 326 | Cyto | |
| 162c | Seq. ID No. 331 | Seq. ID No. 332 | IM | 5 |
| 162d | Seq. ID No. 333 | Seq. ID No. 334 | IM | 5 |
| 162e | Seq. ID No. 335 | Seq. ID No. 336 | IM | 5 |
| 163 | Seq. ID No. 337 | Seq. ID No. 338 | Cyto | 0 |
| 164 | Seq. ID No. 339 | Seq. ID No. 340 | Cyto | |
| 165a | Seq. ID No. 341 | Seq. ID No. 342 | IM | |
| 165b | Seq. ID No. 343 | Seq. ID No. 344 | IM | |
| 167 | Seq. ID No. 345 | Seq. ID No. 346 | IM | |
| 169 | Seq. ID No. 347 | Seq. ID No. 348 | IM | 2 |
| 170 | Seq. ID No. 349 | Seq. ID No. 350 | IM | 1 |
| 171a | Seq. ID No. 351 | Seq. ID No. 352 | Cyto | |
| 171b | Seq. ID No. 353 | Seq. ID No. 354 | Cyto | |
| 172 | Seq. ID No. 355 | Seq. ID No. 356 | Cyto | |
| 175 | Seq. ID No. 361 | Seq. ID No. 362 | Cyto | |
| 176 | Seq. ID No. 363 | Seq. ID No. 364 | Cyto | |
| 177a | Seq. ID No. 365 | Seq. ID No. 366 | IM | |
| 177b | Seq. ID No. 367 | Seq. ID No. 368 | IM | |
| 179a | Seq. ID No. 369 | Seq. ID No. 370 | IM | 2 |
| 179b | Seq. ID No. 371 | Seq. ID No. 372 | IM | 2 |
| 183a | Seq. ID No. 377 | Seq. ID No. 378 | IM | 1 |
| 184 | Seq. ID No. 379 | Seq. ID No. 380 | Cyto | |
| 185 | Seq. ID No. 381 | Seq. ID No. 382 | Cyto | |
| 186 | Seq. ID No. 383 | Seq. ID No. 384 | Cyto | |
| 187a | Seq. ID No. 385 | Seq. ID No. 386 | IM | 1 |
| 187b | Seq. ID No. 387 | Seq. ID No. 388 | IM | 1 |
| 188 | Seq. ID No. 389 | Seq. ID No. 390 | Cyto | |
| 189b | Seq. ID No. 391 | Seq. ID No. 392 | Cyto | |
| 192a | Seq. ID No. 395 | Seq. ID No. 396 | Cyto | 0 |
| 192b | Seq. ID No. 397 | Seq. ID No. 398 | Cyto | 0 |
| 193 | Seq. ID No. 399 | Seq. ID No. 400 | Cyto | |
| 195 | Seq. ID No. 405 | Seq. ID No. 406 | Cyto | |
| 196 | Seq. ID No. 407 | Seq. ID No. 408 | Cyto | 1 |
| 197a | Seq. ID No. 409 | Seq. ID No. 410 | Cyto | |
| 197b | Seq. ID No. 411 | Seq. ID No. 412 | Cyto | |
| 198a | Seq. ID No. 413 | Seq. ID No. 414 | Peri | |
| 198b | Seq. ID No. 415 | Seq. ID No. 416 | Peri | |
| 200 | Seq. ID No. 417 | Seq. ID No. 418 | Cyto | |
| 201a | Seq. ID No. 419 | Seq. ID No. 420 | IM | 2 |
| 202 | Seq. ID No. 421 | Seq. ID No. 422 | Cyto | |
| 203 | Seq. ID No. 423 | Seq. ID No. 424 | Cyto | |
| 204 | Seq. ID No. 425 | Seq. ID No. 426 | Cyto | |
| 205 | Seq. ID No. 427 | Seq. ID No. 428 | Cyto | |
| 206 | Seq. ID No. 429 | Seq. ID No. 430 | Cyto | |
| 207 | Seq. ID No. 431 | Seq. ID No. 432 | IM | 0 |
| 208a | Seq. ID No. 433 | Seq. ID No. 434 | Cyto | |
| 208b | Seq. ID No. 435 | Seq. ID No. 436 | Cyto | |
| 210 | Seq. ID No. 437 | Seq. ID No. 438 | Cyto | |
| 211 | Seq. ID No. 439 | Seq. ID No. 440 | Cyto | |
| 213 | Seq. ID No. 441 | Seq. ID No. 442 | Cyto | |
| 215a | Seq. ID No. 443 | Seq. ID No. 444 | Cyto | |
| 218 | Seq. ID No. 445 | Seq. ID No. 446 | Cyto | |
| 219 | Seq. ID No. 447 | Seq. ID No. 448 | Cyto | 0 |
| 220a | Seq. ID No. 449 | Seq. ID No. 450 | Cyto | 0 |
| 220b | Seq. ID No. 451 | Seq. ID No. 452 | Cyto | 0 |
| 220c | Seq. ID No. 453 | Seq. ID No. 454 | Cyto | 0 |
| 226a | Seq. ID No. 459 | Seq. ID No. 460 | Cyto | |
| 226b | Seq. ID No. 461 | Seq. ID No. 462 | Cyto | |
| 227 | Seq. ID No. 463 | Seq. ID No. 464 | Cyto | |
| 228a | Seq. ID No. 465 | Seq. ID No. 466 | Cyto | |
| 228b | Seq. ID No. 467 | Seq. ID No. 468 | Cyto | |
| 228c | Seq. ID No. 469 | Seq. ID No. 470 | Cyto | |
| 228d | Seq. ID No. 471 | Seq. ID No. 472 | Cyto | |
| 232a | Seq. ID No. 473 | Seq. ID No. 474 | IM | 0 |
| 232b | Seq. ID No. 475 | Seq. ID No. 476 | IM | 0 |
| 235b | Seq. ID No. 479 | Seq. ID No. 480 | Cyto | 1 |
| 236a | Seq. ID No. 481 | Seq. ID No. 482 | Cyto | |
| 236b | Seq. ID No. 483 | Seq. ID No. 484 | Cyto | |
| 236c | Seq. ID No. 485 | Seq. ID No. 486 | Cyto | |
| 245c | Seq. ID No. 501 | Seq. ID No. 502 | IM | 10 |
| 247 | Seq. ID No. 507 | Seq. ID No. 508 | IM | 3 |
| 251 | Seq. ID No. 517 | Seq. ID No. 518 | IM | 1 |
| 253a | Seq. ID No. 519 | Seq. ID No. 520 | IM | |
| 253b | Seq. ID No. 521 | Seq. ID No. 522 | Cyto | |
| 254 | Seq. ID No. 523 | Seq. ID No. 524 | IM | 1 |
| 255c | Seq. ID No. 529 | Seq. ID No. 530 | IM | 12 |
| 255d | Seq. ID No. 531 | Seq. ID No. 532 | IM | 12 |
| 258 | Seq. ID No. 533 | Seq. ID No. 534 | Cyto | |
| 259a | Seq. ID No. 535 | Seq. ID No. 536 | Cyto | |
| 259b | Seq. ID No. 537 | Seq. ID No. 538 | Cyto | |
| 259c | Seq. ID No. 539 | Seq. ID No. 540 | Cyto | |
| 260 | Seq. ID No. 541 | Seq. ID No. 542 | Cyto | |
| 262 | Seq. ID No. 543 | Seq. ID No. 544 | Cyto | |
| 265 | Seq. ID No. 551 | Seq. ID No. 552 | Cyto | |
| 267b | Seq. ID No. 553 | Seq. ID No. 554 | Cyto | 0 |
| 267c | Seq. ID No. 555 | Seq. ID No. 556 | Cyto | 0 |
| 269a | Seq. ID No. 559 | Seq. ID No. 560 | Cyto | |
| 269b | Seq. ID No. 561 | Seq. ID No. 562 | IM | 0 |
| 271a | Seq. ID No. 563 | Seq. ID No. 564 | Cyto | |
| 271b | Seq. ID No. 565 | Seq. ID No. 566 | Cyto | |
| 277a | Seq. ID No. 571 | Seq. ID No. 572 | Cyto | |
| 277b | Seq. ID No. 573 | Seq. ID No. 574 | IM | |
| 280a | Seq. ID No. 581 | Seq. ID No. 582 | Cyto | 1 |
| 280b | Seq. ID No. 583 | Seq. ID No. 584 | Cyto | 1 |
| 280c | Seq. ID No. 585 | Seq. ID No. 586 | Cyto | 1 |
| 280d | Seq. ID No. 587 | Seq. ID No. 588 | Cyto | 1 |
| 282 | Seq. ID No. 589 | Seq. ID No. 590 | Cyto | |
| 284a | Seq. ID No. 595 | Seq. ID No. 596 | IM | 5 |
| 284b | Seq. ID No. 597 | Seq. ID No. 598 | IM | 5 |
| 286a | Seq. ID No. 601 | Seq. ID No. 602 | Cyto | |
| 286b | Seq. ID No. 603 | Seq. ID No. 604 | Cyto | |
| 287 | Seq. ID No. 605 | Seq. ID No. 606 | Cyto | |
| 290a | Seq. ID No. 607 | Seq. ID No. 608 | Cyto | |
| 290b | Seq. ID No. 609 | Seq. ID No. 610 | IM | |
| 293 | Seq. ID No. 611 | Seq. ID No. 612 | Cyto | |
| 294 | Seq. ID No. 613 | Seq. ID No. 614 | Cyto | 0 |
| 296 | Seq. ID No. 615 | Seq. ID No. 616 | Cyto | 0 |
| 300b | Seq. ID No. 619 | Seq. ID No. 620 | IM | 0 |
| 301a | Seq. ID No. 621 | Seq. ID No. 622 | Cyto | 1 |
| 301b | Seq. ID No. 623 | Seq. ID No. 624 | Cyto | 1 |
| 304a | Seq. ID No. 625 | Seq. ID No. 626 | Cyto | 1 |
| 304b | Seq. ID No. 627 | Seq. ID No. 628 | Cyto | 1 |
| 307 | Seq. ID No. 629 | Seq. ID No. 630 | Cyto | |
| 308 | Seq. ID No. 631 | Seq. ID No. 632 | Cyto | 0 |
| 309 | Seq. ID No. 633 | Seq. ID No. 634 | IM | 0 |
| 310 | Seq. ID No. 635 | Seq. ID No. 636 | Cyto | 0 |
| 315 | Seq. ID No. 649 | Seq. ID No. 650 | Cyto | |
| 316a | Seq. ID No. 651 | Seq. ID No. 652 | IM | 3 |
| 318d | Seq. ID No. 663 | Seq. ID No. 664 | IM | 2 |
| 318e | Seq. ID No. 665 | Seq. ID No. 666 | IM | 2 |
| 323a | Seq. ID No. 671 | Seq. ID No. 672 | Cyto | |
| 323b | Seq. ID No. 673 | Seq. ID No. 674 | Cyto | |
| 327c | Seq. ID No. 681 | Seq. ID No. 682 | IM | 5 |
| 329 | Seq. ID No. 683 | Seq. ID No. 684 | Cyto | |
| 330a | Seq. ID No. 685 | Seq. ID No. 686 | Cyto | 0 |
| 330b | Seq. ID No. 687 | Seq. ID No. 688 | Cyto | 0 |
| 330c | Seq. ID No. 689 | Seq. ID No. 690 | Cyto | 0 |
| 331 | Seq. ID No. 691 | Seq. ID No. 692 | Cyto | |
| 332 | Seq. ID No. 693 | Seq. ID No. 694 | Cyto | |
| 333 | Seq. ID No. 695 | Seq. ID No. 696 | Cyto | 1 |
| 334 | Seq. ID No. 697 | Seq. ID No. 698 | Cyto | |
| 335a | Seq. ID No. 699 | Seq. ID No. 700 | IM | 5 |
| 335b | Seq. ID No. 701 | Seq. ID No. 702 | IM | 5 |
| 337 | Seq. ID No. 703 | Seq. ID No. 704 | Cyto | |
| 338a | Seq. ID No. 705 | Seq. ID No. 706 | Cyto | |
| 338b | Seq. ID No. 707 | Seq. ID No. 708 | Cyto | |
| 338c | Seq. ID No. 709 | Seq. ID No. 710 | Cyto | |
| 341a | Seq. ID No. 711 | Seq. ID No. 712 | Cyto | 0 |
| 341b | Seq. ID No. 713 | Seq. ID No. 714 | Cyto | 0 |
| 342g | Seq. ID No. 727 | Seq. ID No. 728 | IM | 12 |
| 342h | Seq. ID No. 729 | Seq. ID No. 730 | IM | 12 |
| 344a | Seq. ID No. 731 | Seq. ID No. 732 | IM | 1 |
| 344b | Seq. ID No. 733 | Seq. ID No. 734 | IM | 1 |
| 345 | Seq. ID No. 735 | Seq. ID No. 736 | Cyto | |
| 346a | Seq. ID No. 737 | Seq. ID No. 738 | Cyto | |
| 346b | Seq. ID No. 739 | Seq. ID No. 740 | Cyto | 0 |
| 347 | Seq. ID No. 741 | Seq. ID No. 742 | Cyto | |
| 349a | Seq. ID No. 743 | Seq. ID No. 744 | Cyto | |
| 351 | Seq. ID No. 753 | Seq. ID No. 754 | Cyto | 1 |
| 355 | Seq. ID No. 767 | Seq. ID No. 768 | Cyto | 0 |
| 356b | Seq. ID No. 771 | Seq. ID No. 772 | IM | 4 |
| 356c | Seq. ID No. 773 | Seq. ID No. 774 | IM | 4 |
| 356d | Seq. ID No. 775 | Seq. ID No. 776 | IM | 5 |
| 362 | Seq. ID No. 783 | Seq. ID No. 784 | IM | 2 |
| 365a | Seq. ID No. 785 | Seq. ID No. 786 | Cyto | |
| 365b | Seq. ID No. 787 | Seq. ID No. 788 | Cyto | |
| 366b | Seq. ID No. 789 | Seq. ID No. 790 | Cyto | |
| 366c | Seq. ID No. 791 | Seq. ID No. 792 | Cyto | |
| 367a | Seq. ID No. 793 | Seq. ID No. 794 | Cyto | 1 |
| 367b | Seq. ID No. 795 | Seq. ID No. 796 | Cyto | 1 |
| 371a | Seq. ID No. 797 | Seq. ID No. 798 | Cyto | |
| 371b | Seq. ID No. 799 | Seq. ID No. 800 | Cyto | |
| 372a | Seq. ID No. 801 | Seq. ID No. 802 | Cyto | |
| 372b | Seq. ID No. 803 | Seq. ID No. 804 | Cyto | |
| 372c | Seq. ID No. 805 | Seq. ID No. 806 | OM | |
| 377a | Seq. ID No. 811 | Seq. ID No. 812 | Cyto | 1 |
| 377b | Seq. ID No. 813 | Seq. ID No. 814 | Cyto | 1 |
| 378a | Seq. ID No. 815 | Seq. ID No. 816 | IM | 1 |
| 378b | Seq. ID No. 817 | Seq. ID No. 818 | IM | 1 |
| 383a | Seq. ID No. 821 | Seq. ID No. 822 | Cyto | |
| 383b | Seq. ID No. 823 | Seq. ID No. 824 | Cyto | 1 |
| 384b | Seq. ID No. 827 | Seq. ID No. 828 | IM | 10 |
| 385a | Seq. ID No. 829 | Seq. ID No. 830 | IM | 0 |
| 385b | Seq. ID No. 831 | Seq. ID No. 832 | IM | 0 |
| 386 | Seq. ID No. 833 | Seq. ID No. 834 | Cyto | |
| 387 | Seq. ID No. 835 | Seq. ID No. 836 | Cyto | |
| 390 | Seq. ID No. 837 | Seq. ID No. 838 | IM | |
| 391 | Seq. ID No. 839 | Seq. ID No. 840 | Peri | |
| 393b | Seq. ID No. 841 | Seq. ID No. 842 | Cyto | |
| 395a | Seq. ID No. 843 | Seq. ID No. 844 | IM | 6 |
| 395b | Seq. ID No. 845 | Seq. ID No. 846 | IM | 6 |
| 395c | Seq. ID No. 847 | Seq. ID No. 848 | IM | 6 |
| 396 | Seq. ID No. 849 | Seq. ID No. 850 | Peri | 2 |
| 398 | Seq. ID No. 851 | Seq. ID No. 852 | IM | 2 |
| 400a | Seq. ID No. 853 | Seq. ID No. 854 | IM | 0 |
| 400b | Seq. ID No. 855 | Seq. ID No. 856 | IM | 0 |
| 401a | Seq. ID No. 857 | Seq. ID No. 858 | Cyto | |
| 401b | Seq. ID No. 859 | Seq. ID No. 860 | Cyto | |
| 403a | Seq. ID No. 861 | Seq. ID No. 862 | Cyto | 1 |
| 403c | Seq. ID No. 865 | Seq. ID No. 866 | Cyto | 2 |
| 403d | Seq. ID No. 867 | Seq. ID No. 868 | Cyto | 2 |
| 406a | Seq. ID No. 869 | Seq. ID No. 870 | Cyto | |
| 406b | Seq. ID No. 871 | Seq. ID No. 872 | Cyto | |
| 409a | Seq. ID No. 873 | Seq. ID No. 874 | Cyto | 0 |
| 409b | Seq. ID No. 875 | Seq. ID No. 876 | Cyto | 0 |
| 410a | Seq. ID No. 877 | Seq. ID No. 878 | Cyto | 0 |
| 410b | Seq. ID No. 879 | Seq. ID No. 880 | IM | 1 |
| 410c | Seq. ID No. 881 | Seq. ID No. 882 | IM | 1 |
| 413a | Seq. ID No. 883 | Seq. ID No. 884 | Cyto | |
| 413b | Seq. ID No. 885 | Seq. ID No. 886 | Cyto | |
| 416a | Seq. ID No. 887 | Seq. ID No. 888 | Cyto | |
| 416b | Seq. ID No. 889 | Seq. ID No. 890 | Cyto | |
| 417 | Seq. ID No. 891 | Seq. ID No. 892 | Cyto | |
| 418b | Seq. ID No. 895 | Seq. ID No. 896 | IM | 7 |
| 419a | Seq. ID No. 897 | Seq. ID No. 898 | IM | 1 |
| 419b | Seq. ID No. 899 | Seq. ID No. 900 | IM | 1 |
| 420a | Seq. ID No. 903 | Seq. ID No. 904 | IM | 1 |
| 420b | Seq. ID No. 905 | Seq. ID No. 906 | IM | 1 |
| 421 | Seq. ID No. 907 | Seq. ID No. 908 | IM | 1 |
| 422 | Seq. ID No. 909 | Seq. ID No. 910 | Cyto | |
| 424a | Seq. ID No. 911 | Seq. ID No. 912 | Cyto | 0 |
| 424b | Seq. ID No. 913 | Seq. ID No. 914 | IM | 1 |
| 424c | Seq. ID No. 915 | Seq. ID No. 916 | IM | 1 |
| 426a | Seq. ID No. 917 | Seq. ID No. 918 | Cyto | |
| 426b | Seq. ID No. 919 | Seq. ID No. 920 | Cyto | |
| 427a | Seq. ID No. 921 | Seq. ID No. 922 | Cyto | |
| 427b | Seq. ID No. 923 | Seq. ID No. 924 | Cyto | |
| 427c | Seq. ID No. 925 | Seq. ID No. 926 | Cyto | |
| 429a | Seq. ID No. 927 | Seq. ID No. 928 | Cyto | |
| 429b | Seq. ID No. 929 | Seq. ID No. 930 | OM | 0 |
| 429c | Seq. ID No. 931 | Seq. ID No. 932 | Cyto | 0 |
| 429d | Seq. ID No. 933 | Seq. ID No. 934 | Cyto | 0 |
| 435 | Seq. ID No. 939 | Seq. ID No. 940 | Cyto | |
| 438a | Seq. ID No. 945 | Seq. ID No. 946 | IM | 1 |
| 438b | Seq. ID No. 947 | Seq. ID No. 948 | IM | 1 |
| 438c | Seq. ID No. 949 | Seq. ID No. 950 | IM | 1 |
| 440 | Seq. ID No. 951 | Seq. ID No. 952 | IM | 2 |
| 441a | Seq. ID No. 953 | Seq. ID No. 954 | IM | 1 |
| 444a | Seq. ID No. 959 | Seq. ID No. 960 | Cyto | |
| 444b | Seq. ID No. 961 | Seq. ID No. 962 | Cyto | |
| 445a | Seq. ID No. 963 | Seq. ID No. 964 | Cyto | 0 |
| 445b | Seq. ID No. 965 | Seq. ID No. 966 | Cyto | 0 |
| 450a | Seq. ID No. 969 | Seq. ID No. 970 | Cyto | 0 |
| 450b | Seq. ID No. 971 | Seq. ID No. 972 | Cyto | 0 |
| 451a | Seq. ID No. 973 | Seq. ID No. 974 | Peri | |
| 451b | Seq. ID No. 975 | Seq. ID No. 976 | Peri | |
| 452a | Seq. ID No. 977 | Seq. ID No. 978 | IM | 4 |
| 454b | Seq. ID No. 987 | Seq. ID No. 988 | Cyto | |
| 455a | Seq. ID No. 989 | Seq. ID No. 990 | IM | 0 |
| 455b | Seq. ID No. 991 | Seq. ID No. 992 | IM | 0 |
| 455c | Seq. ID No. 993 | Seq. ID No. 994 | IM | 0 |
| 458a | Seq. ID No. 995 | Seq. ID No. 996 | Cyto | |
| 458b | Seq. ID No. 997 | Seq. ID No. 998 | Cyto | |
| 460a | Seq. ID No. 999 | Seq. ID No. 1000 | Cyto | 1 |
| 464d | Seq. ID No. 1017 | Seq. ID No. 1018 | IM | 13 |
| 467a | Seq. ID No. 1021 | Seq. ID No. 1022 | Cyto | 0 |
| 467b | Seq. ID No. 1023 | Seq. ID No. 1024 | Cyto | 0 |
| 471a | Seq. ID No. 1025 | Seq. ID No. 1026 | Cyto | 1 |
| 471b | Seq. ID No. 1027 | Seq. ID No. 1028 | Cyto | 1 |
| 473 | Seq. ID No. 1041 | Seq. ID No. 1042 | IM | 2 |
| 474 | Seq. ID No. 1043 | Seq. ID No. 1044 | Cyto | 0 |
| 477b | Seq. ID No. 1047 | Seq. ID No. 1048 | Cyto | 0 |
| 478a | Seq. ID No. 1049 | Seq. ID No. 1050 | IM | 1 |
| 478b | Seq. ID No. 1051 | Seq. ID No. 1052 | IM | 1 |
| 479 | Seq. ID No. 1053 | Seq. ID No. 1054 | Cyto | |
| 480b | Seq. ID No. 1057 | Seq. ID No. 1058 | Cyto | 1 |
| 481a | Seq. ID No. 1059 | Seq. ID No. 1060 | Cyto | |
| 481b | Seq. ID No. 1061 | Seq. ID No. 1062 | Cyto | |
| 482a | Seq. ID No. 1063 | Seq. ID No. 1064 | Cyto | |
| 482b | Seq. ID No. 1065 | Seq. ID No. 1066 | Cyto | |
| 483 | Seq. ID No. 1067 | Seq. ID No. 1068 | Cyto | |
| 490 | Seq. ID No. 1069 | Seq. ID No. 1070 | Cyto | |
| 491a | Seq. ID No. 1071 | Seq. ID No. 1072 | Cyto | |
| 491b | Seq. ID No. 1073 | Seq. ID No. 1074 | Cyto | |
| 495a | Seq. ID No. 1079 | Seq. ID No. 1080 | Cyto | 0 |
| 495b | Seq. ID No. 1081 | Seq. ID No. 1082 | Cyto | 0 |
| 495c | Seq. ID No. 1083 | Seq. ID No. 1084 | IM | 0 |
| 498a | Seq. ID No. 1085 | Seq. ID No. 1086 | Cyto | |
| 498b | Seq. ID No. 1087 | Seq. ID No. 1088 | Cyto | |
| 499a | Seq. ID No. 1089 | Seq. ID No. 1090 | IM | 2 |
| 499b | Seq. ID No. 1091 | Seq. ID No. 1092 | IM | 2 |
| 500 | Seq. ID No. 1093 | Seq. ID No. 1094 | Cyto | |
| 502b | Seq. ID No. 1095 | Seq. ID No. 1096 | Cyto | |
| 502c | Seq. ID No. 1097 | Seq. ID No. 1098 | Cyto | |
| 504 | Seq. ID No. 1099 | Seq. ID No. 1100 | Cyto | |
| 505a | Seq. ID No. 1101 | Seq. ID No. 1102 | Cyto | 1 |
| 505b | Seq. ID No. 1103 | Seq. ID No. 1104 | Cyto | 1 |
| 509 | Seq. ID No. 1109 | Seq. ID No. 1110 | Cyto | |
| 510 | Seq. ID No. 1111 | Seq. ID No. 1112 | Cyto | |
| 512a | Seq. ID No. 1113 | Seq. ID No. 1114 | Cyto | |
| 512b | Seq. ID No. 1115 | Seq. ID No. 1116 | Cyto | |
| 514 | Seq. ID No. 1117 | Seq. ID No. 1118 | Cyto | |
| 515a | Seq. ID No. 1119 | Seq. ID No. 1120 | Cyto | |
| 515b | Seq. ID No. 1121 | Seq. ID No. 1122 | IM | |
| 515c | Seq. ID No. 1123 | Seq. ID No. 1124 | Cyto | |
| 518a | Seq. ID No. 1125 | Seq. ID No. 1126 | Cyto | 0 |
| 518b | Seq. ID No. 1127 | Seq. ID No. 1128 | Cyto | 0 |
| 518c | Seq. ID No. 1129 | Seq. ID No. 1130 | Cyto | 0 |
| 523 | Seq. ID No. 1133 | Seq. ID No. 1134 | Cyto | |
| 527a | Seq. ID No. 1141 | Seq. ID No. 1142 | Cyto | |
| 527b | Seq. ID No. 1143 | Seq. ID No. 1144 | Cyto | |
| 528a | Seq. ID No. 1145 | Seq. ID No. 1146 | IM | 1 |
| 528b | Seq. ID No. 1147 | Seq. ID No. 1148 | IM | 1 |
| 529 | Seq. ID No. 1149 | Seq. ID No. 1150 | Cyto | 0 |
| 530b | Seq. ID No. 1153 | Seq. ID No. 1154 | Peri | |
| 533 | Seq. ID No. 1155 | Seq. ID No. 1156 | IM | 9 |
| 535 | Seq. ID No. 1157 | Seq. ID No. 1158 | Cyto | |
| 536 | Seq. ID No. 1159 | Seq. ID No. 1160 | Cyto | 1 |
| 539b | Seq. ID No. 1161 | Seq. ID No. 1162 | Cyto | |
| 539c | Seq. ID No. 1163 | Seq. ID No. 1164 | Cyto | |
| 541d | Seq. ID No. 1171 | Seq. ID No. 1172 | IM | 5 |
| 543a | Seq. ID No. 1175 | Seq. ID No. 1176 | Cyto | |
| 543b | Seq. ID No. 1177 | Seq. ID No. 1178 | Cyto | |
| 543c | Seq. ID No. 1179 | Seq. ID No. 1180 | Cyto | |
| 544 | Seq. ID No. 1181 | Seq. ID No. 1182 | IM | 4 |
| 545b | Seq. ID No. 1185 | Seq. ID No. 1186 | IM | 3 |
| 545c | Seq. ID No. 1187 | Seq. ID No. 1188 | IM | 4 |
| 546a | Seq. ID No. 1189 | Seq. ID No. 1190 | Cyto | |
| 546b | Seq. ID No. 1191 | Seq. ID No. 1192 | IM | 1 |
| 546c | Seq. ID No. 1193 | Seq. ID No. 1194 | IM | 1 |
| 548 | Seq. ID No. 1199 | Seq. ID No. 1200 | IM | 2 |
| 549b | Seq. ID No. 1201 | Seq. ID No. 1202 | IM | 2 |
| 551 | Seq. ID No. 1205 | Seq. ID No. 1206 | IM | 1 |
| 553c | Seq. ID No. 1207 | Seq. ID No. 1208 | IM | 1 |
| 554a | Seq. ID No. 1209 | Seq. ID No. 1210 | Cyto | 1 |
| 554b | Seq. ID No. 1211 | Seq. ID No. 1212 | Cyto | 1 |
| 557a | Seq. ID No. 1215 | Seq. ID No. 1216 | Cyto | 0 |
| 557b | Seq. ID No. 1217 | Seq. ID No. 1218 | Cyto | 0 |
| 560a | Seq. ID No. 1219 | Seq. ID No. 1220 | IM | 0 |
| 560b | Seq. ID No. 1221 | Seq. ID No. 1222 | IM | 0 |
| 563 | Seq. ID No. 1225 | Seq. ID No. 1226 | IM | 0 |
| 564c | Seq. ID No. 1231 | Seq. ID No. 1232 | IM | 5 |
| 569 | Seq. ID No. 1233 | Seq. ID No. 1234 | Cyto | |
| 570a | Seq. ID No. 1235 | Seq. ID No. 1236 | IM | 1 |
| 571 | Seq. ID No. 1239 | Seq. ID No. 1240 | Peri | 0 |
| 573 | Seq. ID No. 1241 | Seq. ID No. 1242 | IM | 5 |
| 578a | Seq. ID No. 1247 | Seq. ID No. 1248 | Cyto | 0 |
| 578b | Seq. ID No. 1249 | Seq. ID No. 1250 | Cyto | 0 |
| 580a | Seq. ID No. 1251 | Seq. ID No. 1252 | Cyto | |
| 580b | Seq. ID No. 1253 | Seq. ID No. 1254 | Cyto | |
| 583a | Seq. ID No. 1255 | Seq. ID No. 1256 | IM | 0 |
| 583b | Seq. ID No. 1257 | Seq. ID No. 1258 | IM | 0 |
| 586a | Seq. ID No. 1261 | Seq. ID No. 1262 | IM | 2 |
| 586b | Seq. ID No. 1263 | Seq. ID No. 1264 | IM | 2 |
| 588a | Seq. ID No. 1273 | Seq. ID No. 1274 | Cyto | 1 |
| 588b | Seq. ID No. 1275 | Seq. ID No. 1276 | Cyto | 1 |
| 589a | Seq. ID No. 1277 | Seq. ID No. 1278 | Cyto | 1 |
| 589b | Seq. ID No. 1279 | Seq. ID No. 1280 | Cyto | 1 |
| 590 | Seq. ID No. 1281 | Seq. ID No. 1282 | IM | 2 |
| 595b | Seq. ID No. 1285 | Seq. ID No. 1286 | IM | 2 |
| 597 | Seq. ID No. 1293 | Seq. ID No. 1294 | IM | 3 |
| 599a | Seq. ID No. 1299 | Seq. ID No. 1300 | IM | 2 |
| 599b | Seq. ID No. 1301 | Seq. ID No. 1302 | IM | 2 |
| 603a | Seq. ID No. 1307 | Seq. ID No. 1308 | Cyto | 1 |
| 606 | Seq. ID No. 1309 | Seq. ID No. 1310 | Cyto | |
| 607a | Seq. ID No. 1311 | Seq. ID No. 1312 | IM | 0 |
| 607b | Seq. ID No. 1313 | Seq. ID No. 1314 | IM | 0 |
| 610b | Seq. ID No. 1321 | Seq. ID No. 1322 | IM | 1 |
| 611 | Seq. ID No. 1323 | Seq. ID No. 1324 | Peri | 0 |
| 614a | Seq. ID No. 1333 | Seq. ID No. 1334 | Cyto | |
| 614b | Seq. ID No. 1335 | Seq. ID No. 1336 | Cyto | |
| 618 | Seq. ID No. 1339 | Seq. ID No. 1340 | IM | 4 |
| 620 | Seq. ID No. 1345 | Seq. ID No. 1346 | Cyto | |
| 625 | Seq. ID No. 1355 | Seq. ID No. 1356 | Cyto | |
| 626a | Seq. ID No. 1357 | Seq. ID No. 1358 | Cyto | 0 |
| 626b | Seq. ID No. 1359 | Seq. ID No. 1360 | Cyto | 0 |
| 626c | Seq. ID No. 1361 | Seq. ID No. 1362 | Cyto | 0 |
| 631a | Seq. ID No. 1363 | Seq. ID No. 1364 | IM | 1 |
| 633a | Seq. ID No. 1373 | Seq. ID No. 1374 | IM | 0 |
| 633b | Seq. ID No. 1375 | Seq. ID No. 1376 | IM | 0 |
| 633c | Seq. ID No. 1377 | Seq. ID No. 1378 | Cyto | 0 |
| 634b | Seq. ID No. 1379 | Seq. ID No. 1380 | Cyto | |
| 637a | Seq. ID No. 1383 | Seq. ID No. 1384 | IM | 0 |
| 637b | Seq. ID No. 1385 | Seq. ID No. 1386 | IM | 0 |
| 637c | Seq. ID No. 1387 | Seq. ID No. 1388 | IM | 0 |
| 640 | Seq. ID No. 1389 | Seq. ID No. 1390 | Cyto | 0 |
| 641a | Seq. ID No. 1391 | Seq. ID No. 1392 | IM | 1 |
| 641b | Seq. ID No. 1393 | Seq. ID No. 1394 | IM | 1 |
| 643 | Seq. ID No. 1395 | Seq. ID No. 1396 | IM | 1 |
| 644a | Seq. ID No. 1397 | Seq. ID No. 1398 | Cyto | |
| 644b | Seq. ID No. 1399 | Seq. ID No. 1400 | Cyto | |
| 650 | Seq. ID No. 1403 | Seq. ID No. 1404 | Cyto | |
| 652 | Seq. ID No. 1405 | Seq. ID No. 1406 | Cyto | |
| 653a | Seq. ID No. 1407 | Seq. ID No. 1408 | Cyto | 1 |
| 653b | Seq. ID No. 1409 | Seq. ID No. 1410 | Cyto | 1 |
| 653c | Seq. ID No. 1411 | Seq. ID No. 1412 | Cyto | 1 |
| 656 | Seq. ID No. 1413 | Seq. ID No. 1414 | Cyto | 0 |
| 657c | Seq. ID No. 1415 | Seq. ID No. 1416 | Cyto | |
| 658a | Seq. ID No. 1417 | Seq. ID No. 1418 | IM | 4 |
| 658b | Seq. ID No. 1419 | Seq. ID No. 1420 | IM | 4 |
| 659a | Seq. ID No. 1421 | Seq. ID No. 1422 | Cyto | |
| 659b | Seq. ID No. 1423 | Seq. ID No. 1424 | Cyto | |
| 661 | Seq. ID No. 1425 | Seq. ID No. 1426 | Cyto | |
| 663 | Seq. ID No. 1427 | Seq. ID No. 1428 | Cyto | 0 |
| 666 | Seq. ID No. 1431 | Seq. ID No. 1432 | IM | 4 |
| 670 | Seq. ID No. 1433 | Seq. ID No. 1434 | Cyto | 1 |
| 674b | Seq. ID No. 1437 | Seq. ID No. 1438 | IM | 1 |
| 676 | Seq. ID No. 1439 | Seq. ID No. 1440 | Cyto | |
| 677a | Seq. ID No. 1441 | Seq. ID No. 1442 | Cyto | 0 |
| 677b | Seq. ID No. 1443 | Seq. ID No. 1444 | Cyto | 0 |
| 680 | Seq. ID No. 1449 | Seq. ID No. 1450 | Cyto | 0 |
| 681 | Seq. ID No. 1451 | Seq. ID No. 1452 | Cyto | 0 |
| 683a | Seq. ID No. 1453 | Seq. ID No. 1454 | Cyto | |
| 683b | Seq. ID No. 1455 | Seq. ID No. 1456 | Cyto | |
| 684 | Seq. ID No. 1457 | Seq. ID No. 1458 | Cyto | |
| 687 | Seq. ID No. 1459 | Seq. ID No. 1460 | Peri | |
| 688a | Seq. ID No. 1461 | Seq. ID No. 1462 | IM | 4 |
| 688b | Seq. ID No. 1463 | Seq. ID No. 1464 | IM | 4 |
| 690 | Seq. ID No. 1465 | Seq. ID No. 1466 | Cyto | |
| 691a | Seq. ID No. 1467 | Seq. ID No. 1468 | Cyto | |
| 691b | Seq. ID No. 1469 | Seq. ID No. 1470 | Cyto | |
| 692 | Seq. ID No. 1471 | Seq. ID No. 1472 | IM | 0 |
| 694 | Seq. ID No. 1473 | Seq. ID No. 1474 | Cyto | |
| 696 | Seq. ID No. 1475 | Seq. ID No. 1476 | Cyto | |
| 698 | Seq. ID No. 1477 | Seq. ID No. 1478 | IM | 4 |
| 699b | Seq. ID No. 1479 | Seq. ID No. 1480 | IM | 1 |
| 700b | Seq. ID No. 1483 | Seq. ID No. 1484 | IM | 7 |
| 700c | Seq. ID No. 1485 | Seq. ID No. 1486 | IM | 7 |
| 703 | Seq. ID No. 1487 | Seq. ID No. 1488 | IM | 0 |
| 705a | Seq. ID No. 1489 | Seq. ID No. 1490 | Cyto | 0 |
| 705b | Seq. ID No. 1491 | Seq. ID No. 1492 | Cyto | 0 |
| 707 | Seq. ID No. 1493 | Seq. ID No. 1494 | Cyto | |
| 709 | Seq. ID No. 1495 | Seq. ID No. 1496 | Cyto | |
| 710 | Seq. ID No. 1497 | Seq. ID No. 1498 | Cyto | |
| 712a | Seq. ID No. 1499 | Seq. ID No. 1500 | Cyto | |
| 712b | Seq. ID No. 1501 | Seq. ID No. 1502 | Cyto | |
| 715a | Seq. ID No. 1503 | Seq. ID No. 1504 | Cyto | 0 |
| 715b | Seq. ID No. 1505 | Seq. ID No. 1506 | Cyto | 0 |
| 717 | Seq. ID No. 1507 | Seq. ID No. 1508 | Cyto | |
| 718a | Seq. ID No. 1509 | Seq. ID No. 1510 | Cyto | |
| 721 | Seq. ID No. 1511 | Seq. ID No. 1512 | Cyto | |
| 724a | Seq. ID No. 1519 | Seq. ID No. 1520 | Cyto | 0 |
| 724b | Seq. ID No. 1521 | Seq. ID No. 1522 | Cyto | 0 |
| 724c | Seq. ID No. 1523 | Seq. ID No. 1524 | Cyto | 0 |
| 724d | Seq. ID No. 1525 | Seq. ID No. 1526 | Cyto | 0 |
| 726 | Seq. ID No. 1527 | Seq. ID No. 1528 | Cyto | |
| 728b | Seq. ID No. 1531 | Seq. ID No. 1532 | IM | 8 |
| 730c | Seq. ID No. 1537 | Seq. ID No. 1538 | IM | 5 |
| 730d | Seq. ID No. 1539 | Seq. ID No. 1540 | IM | 5 |
| 731b | Seq. ID No. 1541 | Seq. ID No. 1542 | Cyto | 1 |
| 732 | Seq. ID No. 1543 | Seq. ID No. 1544 | Cyto | 2 |
| 738c | Seq. ID No. 1549 | Seq. ID No. 1550 | IM | 10 |
| 741a | Seq. ID No. 1555 | Seq. ID No. 1556 | Cyto | |
| 741b | Seq. ID No. 1557 | Seq. ID No. 1558 | Cyto | |
| 745a | Seq. ID No. 1565 | Seq. ID No. 1566 | IM | 6 |
| 745b | Seq. ID No. 1567 | Seq. ID No. 1568 | IM | 6 |
| 746a | Seq. ID No. 1569 | Seq. ID No. 1570 | IM | 6 |
| 747 | Seq. ID No. 1573 | Seq. ID No. 1574 | Cyto | |
| 748a | Seq. ID No. 1575 | Seq. ID No. 1576 | Cyto | |
| 748b | Seq. ID No. 1577 | Seq. ID No. 1578 | Cyto | |
| 751 | Seq. ID No. 1581 | Seq. ID No. 1582 | IM | 1 |
| 754c | Seq. ID No. 1587 | Seq. ID No. 1588 | IM | 9 |
| 754d | Seq. ID No. 1589 | Seq. ID No. 1590 | IM | 9 |
| 756a | Seq. ID No. 1591 | Seq. ID No. 1592 | Cyto | 1 |
| 756b | Seq. ID No. 1593 | Seq. ID No. 1594 | Cyto | 1 |
| 758 | Seq. ID No. 1595 | Seq. ID No. 1596 | Cyto | |
| 760 | Seq. ID No. 1597 | Seq. ID No. 1598 | Cyto | |
| 762b | Seq. ID No. 1601 | Seq. ID No. 1602 | Cyto | 0 |
| 762c | Seq. ID No. 1603 | Seq. ID No. 1604 | Cyto | 0 |
| 763a | Seq. ID No. 1605 | Seq. ID No. 1606 | Cyto | |
| 763b | Seq. ID No. 1607 | Seq. ID No. 1608 | Cyto | |
| 764 | Seq. ID No. 1609 | Seq. ID No. 1610 | Cyto | |
| 766 | Seq. ID No. 1611 | Seq. ID No. 1612 | Cyto | 0 |
| 767 | Seq. ID No. 1613 | Seq. ID No. 1614 | Cyto | |
| 770a | Seq. ID No. 1615 | Seq. ID No. 1616 | IM | |
| 770b | Seq. ID No. 1617 | Seq. ID No. 1618 | Cyto | |
| 770c | Seq. ID No. 1619 | Seq. ID No. 1620 | Cyto | |
| 772b | Seq. ID No. 1623 | Seq. ID No. 1624 | IM | 6 |
| 774 | Seq. ID No. 1625 | Seq. ID No. 1626 | Cyto | 0 |
| 775 | Seq. ID No. 1627 | Seq. ID No. 1628 | IM | 7 |
| 777a | Seq. ID No. 1629 | Seq. ID No. 1630 | Cyto | |
| 777c | Seq. ID No. 1631 | Seq. ID No. 1632 | OM | |
| 780a | Seq. ID No. 1635 | Seq. ID No. 1636 | Cyto | |
| 780b | Seq. ID No. 1637 | Seq. ID No. 1638 | Cyto | |
| 785 | Seq. ID No. 1641 | Seq. ID No. 1642 | Cyto | 0 |
| 789b | Seq. ID No. 1651 | Seq. ID No. 1652 | IM | 4 |
| 791 | Seq. ID No. 1655 | Seq. ID No. 1656 | Cyto | |
| 792 | Seq. ID No. 1657 | Seq. ID No. 1658 | Cyto | |
| 794a | Seq. ID No. 1659 | Seq. ID No. 1660 | Peri | 0 |
| 795 | Seq. ID No. 1667 | Seq. ID No. 1668 | Cyto | |
| 797 | Seq. ID No. 1669 | Seq. ID No. 1670 | Cyto | |
| 799c | Seq. ID No. 1675 | Seq. ID No. 1676 | IM | 9 |
| 803a | Seq. ID No. 1683 | Seq. ID No. 1684 | Cyto | 3 |
| 803b | Seq. ID No. 1685 | Seq. ID No. 1686 | Cyto | 3 |
| 804a | Seq. ID No. 1687 | Seq. ID No. 1688 | Cyto | |
| 804b | Seq. ID No. 1689 | Seq. ID No. 1690 | Cyto | |
| 807 | Seq. ID No. 1691 | Seq. ID No. 1692 | Cyto | |
| 809b | Seq. ID No. 1695 | Seq. ID No. 1696 | IM | 4 |
| 812 | Seq. ID No. 1697 | Seq. ID No. 1698 | Cyto | 0 |
| 813 | Seq. ID No. 1699 | Seq. ID No. 1700 | IM | 5 |
| 816 | Seq. ID No. 1701 | Seq. ID No. 1702 | IM | 0 |
| 820a | Seq. ID No. 1703 | Seq. ID No. 1704 | IM | 0 |
| 820b | Seq. ID No. 1705 | Seq. ID No. 1706 | IM | 0 |
| 829a | Seq. ID No. 1709 | Seq. ID No. 1710 | Peri | |
| 830 | Seq. ID No. 1713 | Seq. ID No. 1714 | Cyto | |
| 834a | Seq. ID No. 1715 | Seq. ID No. 1716 | Cyto | |
| 834b | Seq. ID No. 1717 | Seq. ID No. 1718 | Cyto | |
| 834c | Seq. ID No. 1719 | Seq. ID No. 1720 | Cyto | |
| 835a | Seq. ID No. 1721 | Seq. ID No. 1722 | Cyto | |
| 835b | Seq. ID No. 1723 | Seq. ID No. 1724 | Cyto | |
| 836a | Seq. ID No. 1725 | Seq. ID No. 1726 | Cyto | |
| 836b | Seq. ID No. 1727 | Seq. ID No. 1728 | Cyto | |
| 836c | Seq. ID No. 1729 | Seq. ID No. 1730 | Cyto | |
| 837c | Seq. ID No. 1735 | Seq. ID No. 1736 | IM | 8 |
| 847a | Seq. ID No. 1739 | Seq. ID No. 1740 | IM | 1 |
| 847b | Seq. ID No. 1741 | Seq. ID No. 1742 | IM | 1 |
| 848 | Seq. ID No. 1743 | Seq. ID No. 1744 | Cyto | |
| 849a | Seq. ID No. 1745 | Seq. ID No. 1746 | Cyto | |
| 849b | Seq. ID No. 1747 | Seq. ID No. 1748 | IM | 0 |
| 851a | Seq. ID No. 1749 | Seq. ID No. 1750 | Cyto | |
| 851b | Seq. ID No. 1751 | Seq. ID No. 1752 | Cyto | |
| 854 | Seq. ID No. 1759 | Seq. ID No. 1760 | IM | 2 |
| 855 | Seq. ID No. 1761 | Seq. ID No. 1762 | Cyto | |
| 857 | Seq. ID No. 1763 | Seq. ID No. 1764 | Peri | 1 |
| 859c | Seq. ID No. 1769 | Seq. ID No. 1770 | IM | 6 |
| 860 | Seq. ID No. 1773 | Seq. ID No. 1774 | Cyto | |
| 862 | Seq. ID No. 1775 | Seq. ID No. 1776 | Peri | 0 |
| 863 | Seq. ID No. 1777 | Seq. ID No. 1778 | Cyto | |
| 864a | Seq. ID No. 1779 | Seq. ID No. 1780 | Cyto | 1 |
| 866a | Seq. ID No. 1783 | Seq. ID No. 1784 | Cyto | |
| 866b | Seq. ID No. 1785 | Seq. ID No. 1786 | Cyto | 0 |
| 868b | Seq. ID No. 1789 | Seq. ID No. 1790 | IM | 6 |
| 873a | Seq. ID No. 1793 | Seq. ID No. 1794 | Cyto | |
| 873b | Seq. ID No. 1795 | Seq. ID No. 1796 | Cyto | |
| 874a | Seq. ID No. 1799 | Seq. ID No. 1800 | Cyto | |
| 874b | Seq. ID No. 1801 | Seq. ID No. 1802 | Cyto | |
| 875 | Seq. ID No. 1803 | Seq. ID No. 1804 | Cyto | |
| 878a | Seq. ID No. 1807 | Seq. ID No. 1808 | Cyto | |
| 878b | Seq. ID No. 1809 | Seq. ID No. 1810 | Cyto | |
| 878c | Seq. ID No. 1811 | Seq. ID No. 1812 | Cyto | 0 |
| 880a | Seq. ID No. 1813 | Seq. ID No. 1814 | IM | 0 |
| 880b | Seq. ID No. 1815 | Seq. ID No. 1816 | IM | 0 |
| 880c | Seq. ID No. 1817 | Seq. ID No. 1818 | IM | 0 |
| 888 | Seq. ID No. 1825 | Seq. ID No. 1826 | Cyto | |
| 890a | Seq. ID No. 1827 | Seq. ID No. 1828 | Cyto | |
| 890b | Seq. ID No. 1829 | Seq. ID No. 1830 | Cyto | |
| 894 | Seq. ID No. 1831 | Seq. ID No. 1832 | Cyto | |
| 904 | Seq. ID No. 1833 | Seq. ID No. 1834 | Peri | |
| 907a | Seq. ID No. 1835 | Seq. ID No. 1836 | Cyto | 1 |
| 907b | Seq. ID No. 1837 | Seq. ID No. 1838 | Cyto | 1 |
| 908b | Seq. ID No. 1841 | Seq. ID No. 1842 | IM | 9 |
| 908c | Seq. ID No. 1843 | Seq. ID No. 1844 | IM | 9 |
| 911 | Seq. ID No. 1845 | Seq. ID No. 1846 | Cyto | |
| 912 | Seq. ID No. 1847 | Seq. ID No. 1848 | Cyto | |
| 916 | Seq. ID No. 1849 | Seq. ID No. 1850 | Cyto | 1 |
| 917 | Seq. ID No. 1851 | Seq. ID No. 1852 | Cyto | |
| 918b | Seq. ID No. 1853 | Seq. ID No. 1854 | Cyto | |
| 918c | Seq. ID No. 1855 | Seq. ID No. 1856 | Cyto | |
| 923 | Seq. ID No. 1859 | Seq. ID No. 1860 | Cyto | 0 |
| 925a | Seq. ID No. 1861 | Seq. ID No. 1862 | Cyto | 0 |
| 925b | Seq. ID No. 1863 | Seq. ID No. 1864 | Cyto | 0 |
| 926 | Seq. ID No. 1865 | Seq. ID No. 1866 | Cyto | 0 |
| 927 | Seq. ID No. 1867 | Seq. ID No. 1868 | Cyto | |
| 929b | Seq. ID No. 1871 | Seq. ID No. 1872 | Cyto | 1 |
| 930 | Seq. ID No. 1873 | Seq. ID No. 1874 | Cyto | 1 |
| 932 | Seq. ID No. 1877 | Seq. ID No. 1878 | Cyto | 1 |
| 934a | Seq. ID No. 1879 | Seq. ID No. 1880 | Cyto | |
| 934b | Seq. ID No. 1881 | Seq. ID No. 1882 | Cyto | |
| 934c | Seq. ID No. 1883 | Seq. ID No. 1884 | Cyto | 0 |
| 934d | Seq. ID No. 1885 | Seq. ID No. 1886 | Cyto | 0 |
| 935 | Seq. ID No. 1887 | Seq. ID No. 1888 | Cyto | 0 |
| 938 | Seq. ID No. 1889 | Seq. ID No. 1890 | Cyto | 1 |
| 942 | Seq. ID No. 1897 | Seq. ID No. 1898 | IM | |
| 943 | Seq. ID No. 1899 | Seq. ID No. 1900 | Cyto | |
| 944a | Seq. ID No. 1901 | Seq. ID No. 1902 | Cyto | 0 |
| 944b | Seq. ID No. 1903 | Seq. ID No. 1904 | Cyto | 0 |
| 946 | Seq. ID No. 1905 | Seq. ID No. 1906 | Cyto | 0 |
| 949a | Seq. ID No. 1907 | Seq. ID No. 1908 | Cyto | |
| 949b | Seq. ID No. 1909 | Seq. ID No. 1910 | IM | 1 |
| 949c | Seq. ID No. 1911 | Seq. ID No. 1912 | IM | 1 |
| 949d | Seq. ID No. 1913 | Seq. ID No. 1914 | IM | 1 |
| 952a | Seq. ID No. 1921 | Seq. ID No. 1922 | IM | 1 |
| 954 | Seq. ID No. 1925 | Seq. ID No. 1926 | Cyto | 0 |
| 956 | Seq. ID No. 1931 | Seq. ID No. 1932 | IM | 0 |
| 958a | Seq. ID No. 1933 | Seq. ID No. 1934 | Cyto | |
| 958b | Seq. ID No. 1935 | Seq. ID No. 1936 | Cyto | |
| 959 | Seq. ID No. 1937 | Seq. ID No. 1938 | Cyto | |
| 961a | Seq. ID No. 1939 | Seq. ID No. 1940 | Cyto | |
| 961b | Seq. ID No. 1941 | Seq. ID No. 1942 | Cyto | |
| 961c | Seq. ID No. 1943 | Seq. ID No. 1944 | Cyto | |
| 963 | Seq. ID No. 1945 | Seq. ID No. 1946 | Cyto | 0 |
| 965a | Seq. ID No. 1947 | Seq. ID No. 1948 | Cyto | |
| 965b | Seq. ID No. 1949 | Seq. ID No. 1950 | Cyto | |
| 966a | Seq. ID No. 1951 | Seq. ID No. 1952 | Cyto | |
| 966b | Seq. ID No. 1953 | Seq. ID No. 1954 | Cyto | |
| 967a | Seq. ID No. 1955 | Seq. ID No. 1956 | IM | |
| 967b | Seq. ID No. 1957 | Seq. ID No. 1958 | Cyto | |
| 968 | Seq. ID No. 1959 | Seq. ID No. 1960 | Cyto | 0 |
| 969 | Seq. ID No. 1961 | Seq. ID No. 1962 | IM | |
| 972a | Seq. ID No. 1963 | Seq. ID No. 1964 | IM | |
| 972b | Seq. ID No. 1965 | Seq. ID No. 1966 | IM | |
| 976 | Seq. ID No. 1967 | Seq. ID No. 1968 | Cyto | |
| 977b | Seq. ID No. 1971 | Seq. ID No. 1972 | Cyto | 1 |
| 978a | Seq. ID No. 1973 | Seq. ID No. 1974 | Cyto | |
| 978b | Seq. ID No. 1975 | Seq. ID No. 1976 | Cyto | |
| 979 | Seq. ID No. 1977 | Seq. ID No. 1978 | IM | 1 |
| 980 | Seq. ID No. 1979 | Seq. ID No. 1980 | Cyto | 0 |
| 982 | Seq. ID No. 1983 | Seq. ID No. 1984 | Cyto | |
| 984a | Seq. ID No. 1985 | Seq. ID No. 1986 | Cyto | |
| 984b | Seq. ID No. 1987 | Seq. ID No. 1988 | Cyto | |
| 986 | Seq. ID No. 1989 | Seq. ID No. 1990 | Cyto | |
| 988b | Seq. ID No. 1995 | Seq. ID No. 1996 | IM | 6 |
| 988c | Seq. ID No. 1997 | Seq. ID No. 1998 | IM | 6 |
| 988d | Seq. ID No. 1999 | Seq. ID No. 2000 | IM | 6 |
| 991 | Seq. ID No. 2001 | Seq. ID No. 2002 | IM | |
| 992 | Seq. ID No. 2003 | Seq. ID No. 2004 | Cyto | 0 |
| 994 | Seq. ID No. 2007 | Seq. ID No. 2008 | Cyto | 2 |
| 995 | Seq. ID No. 2009 | Seq. ID No. 2010 | IM | 1 |
| 997 | Seq. ID No. 2011 | Seq. ID No. 2012 | IM | |
| 998b | Seq. ID No. 2015 | Seq. ID No. 2016 | IM | 5 |
| 1000 | Seq. ID No. 2021 | Seq. ID No. 2022 | Cyto | 1 |
| 1001 | Seq. ID No. 2023 | Seq. ID No. 2024 | Cyto | |
| 1004a | Seq. ID No. 2029 | Seq. ID No. 2030 | IM | 1 |
| 1004b | Seq. ID No. 2031 | Seq. ID No. 2032 | IM | 1 |
| 1007 | Seq. ID No. 2037 | Seq. ID No. 2038 | Cyto | |
| 1012 | Seq. ID No. 2039 | Seq. ID No. 2040 | IM | 1 |
| 1013b | Seq. ID No. 2043 | Seq. ID No. 2044 | IM | 6 |
| 1013c | Seq. ID No. 2045 | Seq. ID No. 2046 | IM | 6 |
| 1016c | Seq. ID No. 2047 | Seq. ID No. 2048 | Cyto | |
| 1017b | Seq. ID No. 2051 | Seq. ID No. 2052 | IM | 5 |
| 1018a | Seq. ID No. 2057 | Seq. ID No. 2058 | Cyto | |
| 1018b | Seq. ID No. 2059 | Seq. ID No. 2060 | Cyto | |
| 1021a | Seq. ID No. 2061 | Seq. ID No. 2062 | Cyto | 1 |
| 1021b | Seq. ID No. 2063 | Seq. ID No. 2064 | Cyto | 1 |
| 1021c | Seq. ID No. 2065 | Seq. ID No. 2066 | Cyto | 1 |
| 1024 | Seq. ID No. 2067 | Seq. ID No. 2068 | Cyto | |
| 1025 | Seq. ID No. 2069 | Seq. ID No. 2070 | Cyto | |
| 1026 | Seq. ID No. 2071 | Seq. ID No. 2072 | Cyto | |
| 1029 | Seq. ID No. 2073 | Seq. ID No. 2074 | Cyto | |
| 1033a | Seq. ID No. 2075 | Seq. ID No. 2076 | IM | 0 |
| 1033b | Seq. ID No. 2077 | Seq. ID No. 2078 | IM | 0 |
| 1033c | Seq. ID No. 2079 | Seq. ID No. 2080 | IM | 0 |
| 1035a | Seq. ID No. 2081 | Seq. ID No. 2082 | IM | 1 |
| 1035b | Seq. ID No. 2083 | Seq. ID No. 2084 | IM | 1 |
| 1035c | Seq. ID No. 2085 | Seq. ID No. 2086 | IM | 1 |
| 1037 | Seq. ID No. 2093 | Seq. ID No. 2094 | Cyto | |
| 1039a | Seq. ID No. 2095 | Seq. ID No. 2096 | Cyto | 0 |
| 1039b | Seq. ID No. 2097 | Seq. ID No. 2098 | Cyto | 0 |
| 1041 | Seq. ID No. 2099 | Seq. ID No. 2100 | Cyto | |
| 1042a | Seq. ID No. 2101 | Seq. ID No. 2102 | Cyto | 1 |
| 1042b | Seq. ID No. 2103 | Seq. ID No. 2104 | Cyto | 1 |
| 1044d | Seq. ID No. 2111 | Seq. ID No. 2112 | IM | 3 |
| 1049a | Seq. ID No. 2117 | Seq. ID No. 2118 | Cyto | 0 |
| 1049b | Seq. ID No. 2119 | Seq. ID No. 2120 | Cyto | 0 |
| 1055 | Seq. ID No. 2129 | Seq. ID No. 2130 | Cyto | |
| 1056a | Seq. ID No. 2131 | Seq. ID No. 2132 | Cyto | |
| 1056b | Seq. ID No. 2133 | Seq. ID No. 2134 | Cyto | |
| 1057 | Seq. ID No. 2135 | Seq. ID No. 2136 | Cyto | 0 |
| 1058 | Seq. ID No. 2137 | Seq. ID No. 2138 | Cyto | 1 |
| 1060a | Seq. ID No. 2139 | Seq. ID No. 2140 | IM | 1 |
| 1060b | Seq. ID No. 2141 | Seq. ID No. 2142 | IM | 1 |
| 1062 | Seq. ID No. 2143 | Seq. ID No. 2144 | Cyto | |
| 1063 | Seq. ID No. 2145 | Seq. ID No. 2146 | Cyto | 1 |
| 1064 | Seq. ID No. 2147 | Seq. ID No. 2148 | Cyto | |
| 1065c | Seq. ID No. 2153 | Seq. ID No. 2154 | IM | 1 |
| 1069 | Seq. ID No. 2161 | Seq. ID No. 2162 | IM | 1 |
| 1070a | Seq. ID No. 2163 | Seq. ID No. 2164 | Cyto | |
| 1070b | Seq. ID No. 2165 | Seq. ID No. 2166 | Cyto | |
| 1076a | Seq. ID No. 2167 | Seq. ID No. 2168 | IM | 2 |
| 1076b | Seq. ID No. 2169 | Seq. ID No. 2170 | IM | 2 |
| 1077c | Seq. ID No. 2175 | Seq. ID No. 2176 | IM | 12 |
| 1077e | Seq. ID No. 2179 | Seq. ID No. 2180 | IM | 15 |
| 1079a | Seq. ID No. 2181 | Seq. ID No. 2182 | Cyto | |
| 1079b | Seq. ID No. 2183 | Seq. ID No. 2184 | Cyto | |
| 1080a | Seq. ID No. 2185 | Seq. ID No. 2186 | Cyto | |
| 1081b | Seq. ID No. 2189 | Seq. ID No. 2190 | IM | 1 |
| 1085 | Seq. ID No. 2193 | Seq. ID No. 2194 | Cyto | |
| 1086 | Seq. ID No. 2195 | Seq. ID No. 2196 | IM | 1 |
| 1087a | Seq. ID No. 2197 | Seq. ID No. 2198 | IM | 0 |
| 1087b | Seq. ID No. 2199 | Seq. ID No. 2200 | IM | 0 |
| 1091a | Seq. ID No. 2201 | Seq. ID No. 2202 | Cyto | |
| 1091b | Seq. ID No. 2203 | Seq. ID No. 2204 | Peri | |
| 1094b | Seq. ID No. 2207 | Seq. ID No. 2208 | IM | 2 |
| 1095a | Seq. ID No. 2209 | Seq. ID No. 2210 | Cyto | |
| 1095b | Seq. ID No. 2211 | Seq. ID No. 2212 | Cyto | |
| 1096a | Seq. ID No. 2213 | Seq. ID No. 2214 | Cyto | 0 |
| 1096b | Seq. ID No. 2215 | Seq. ID No. 2216 | Cyto | 0 |
| 1096c | Seq. ID No. 2217 | Seq. ID No. 2218 | Cyto | 0 |
| 1097 | Seq. ID No. 2219 | Seq. ID No. 2220 | IM | 2 |
| 1098b | Seq. ID No. 2221 | Seq. ID No. 2222 | IM | 2 |
| 1100 | Seq. ID No. 2223 | Seq. ID No. 2224 | Cyto | |
| 1101a | Seq. ID No. 2225 | Seq. ID No. 2226 | Cyto | |
| 1101b | Seq. ID No. 2227 | Seq. ID No. 2228 | Cyto | |
| 1104 | Seq. ID No. 2229 | Seq. ID No. 2230 | Cyto | |
| 1106 | Seq. ID No. 2231 | Seq. ID No. 2232 | Cyto | |
| 1108a | Seq. ID No. 2233 | Seq. ID No. 2234 | Cyto | |
| 1108b | Seq. ID No. 2235 | Seq. ID No. 2236 | Cyto | |
| 1111c | Seq. ID No. 2241 | Seq. ID No. 2242 | IM | 8 |
| 1112a | Seq. ID No. 2243 | Seq. ID No. 2244 | IM | 1 |
| 1126a | Seq. ID No. 2247 | Seq. ID No. 2248 | IM | 3 |
| 1127 | Seq. ID No. 2253 | Seq. ID No. 2254 | Cyto | |
| 1131 | Seq. ID No. 2257 | Seq. ID No. 2258 | Cyto | 1 |
| 1135a | Seq. ID No. 2261 | Seq. ID No. 2262 | IM | 1 |
| 1135b | Seq. ID No. 2263 | Seq. ID No. 2264 | IM | 1 |
| 1136a | Seq. ID No. 2265 | Seq. ID No. 2266 | IM | |
| 1136b | Seq. ID No. 2267 | Seq. ID No. 2268 | IM | |
| 1138 | Seq. ID No. 2269 | Seq. ID No. 2270 | Cyto | |
| 1140b | Seq. ID No. 2273 | Seq. ID No. 2274 | IM | 3 |
| 1140c | Seq. ID No. 2275 | Seq. ID No. 2276 | IM | 6 |
| 1141a | Seq. ID No. 2277 | Seq. ID No. 2278 | Cyto | 1 |
| 1141b | Seq. ID No. 2279 | Seq. ID No. 2280 | Cyto | 1 |
| 1142 | Seq. ID No. 2281 | Seq. ID No. 2282 | Cyto | |
| 1144b | Seq. ID No. 2285 | Seq. ID No. 2286 | IM | 4 |
| 1144c | Seq. ID No. 2287 | Seq. ID No. 2288 | IM | 5 |
| 1146e | Seq. ID No. 2297 | Seq. ID No. 2298 | IM | 10 |
| 1148 | Seq. ID No. 2299 | Seq. ID No. 2300 | Cyto | 0 |
| 1149 | Seq. ID No. 2301 | Seq. ID No. 2302 | IM | 0 |
| 1152d | Seq. ID No. 2309 | Seq. ID No. 2310 | IM | 7 |
| 1152e | Seq. ID No. 2311 | Seq. ID No. 2312 | IM | 8 |
| 1152f | Seq. ID No. 2313 | Seq. ID No. 2314 | IM | 8 |
| 1152g | Seq. ID No. 2315 | Seq. ID No. 2316 | IM | 8 |
| 1158a | Seq. ID No. 2331 | Seq. ID No. 2332 | Cyto | |
| 1160 | Seq. ID No. 2335 | Seq. ID No. 2336 | Cyto | |
| 1161a | Seq. ID No. 2337 | Seq. ID No. 2338 | Cyto | |
| 1161b | Seq. ID No. 2339 | Seq. ID No. 2340 | Cyto | |
| 1162 | Seq. ID No. 2341 | Seq. ID No. 2342 | Cyto | |
| 1166 | Seq. ID No. 2345 | Seq. ID No. 2346 | Cyto | |
| 1170 | Seq. ID No. 2359 | Seq. ID No. 2360 | Cyto | 0 |
| 1171a | Seq. ID No. 2361 | Seq. ID No. 2362 | Cyto | |
| 1171c | Seq. ID No. 2363 | Seq. ID No. 2364 | Cyto | |
| 1174 | Seq. ID No. 2369 | Seq. ID No. 2370 | Cyto | 2 |
| 1177 | Seq. ID No. 2373 | Seq. ID No. 2374 | Cyto | 1 |
| 1179a | Seq. ID No. 2377 | Seq. ID No. 2378 | Cyto | 0 |
| 1179b | Seq. ID No. 2379 | Seq. ID No. 2380 | Cyto | 0 |
| 1179c | Seq. ID No. 2381 | Seq. ID No. 2382 | Cyto | 0 |
| 1180a | Seq. ID No. 2383 | Seq. ID No. 2384 | Cyto | 1 |
| 1180b | Seq. ID No. 2385 | Seq. ID No. 2386 | IM | 2 |
| 1182b | Seq. ID No. 2395 | Seq. ID No. 2396 | IM | 2 |
| 1183a | Seq. ID No. 2397 | Seq. ID No. 2398 | IM | 0 |
| 1183b | Seq. ID No. 2399 | Seq. ID No. 2400 | IM | 0 |
| 1184 | Seq. ID No. 2401 | Seq. ID No. 2402 | Cyto | 1 |
| 1185a | Seq. ID No. 2403 | Seq. ID No. 2404 | Cyto | 0 |
| 1185b | Seq. ID No. 2405 | Seq. ID No. 2406 | Cyto | 0 |
| 1186b | Seq. ID No. 2409 | Seq. ID No. 2410 | IM | 5 |
| 1188a | Seq. ID No. 2411 | Seq. ID No. 2412 | IM | 2 |
| 1188b | Seq. ID No. 2413 | Seq. ID No. 2414 | IM | 4 |
| 1188c | Seq. ID No. 2415 | Seq. ID No. 2416 | IM | 4 |
| 1191 | Seq. ID No. 2417 | Seq. ID No. 2418 | IM | 0 |
| 1194 | Seq. ID No. 2423 | Seq. ID No. 2424 | IM | 1 |
| 1198 | Seq. ID No. 2431 | Seq. ID No. 2432 | Cyto | |
| 1199b | Seq. ID No. 2435 | Seq. ID No. 2436 | IM | 3 |
| 1200 | Seq. ID No. 2437 | Seq. ID No. 2438 | Cyto | 0 |
| 1201 | Seq. ID No. 2439 | Seq. ID No. 2440 | Cyto | |
| 1202a | Seq. ID No. 2441 | Seq. ID No. 2442 | IM | 3 |
| 1202b | Seq. ID No. 2443 | Seq. ID No. 2444 | IM | 3 |
| 1205 | Seq. ID No. 2445 | Seq. ID No. 2446 | IM | 3 |
| 1206 | Seq. ID No. 2447 | Seq. ID No. 2448 | Cyto | 0 |
| 1208 | Seq. ID No. 2451 | Seq. ID No. 2452 | Cyto | |
| 1209 | Seq. ID No. 2453 | Seq. ID No. 2454 | IM | 4 |
| 1210a | Seq. ID No. 2455 | Seq. ID No. 2456 | IM | 3 |
| 1210b | Seq. ID No. 2457 | Seq. ID No. 2458 | IM | 4 |
| 1213a | Seq. ID No. 2461 | Seq. ID No. 2462 | Cyto | 1 |
| 1213b | Seq. ID No. 2463 | Seq. ID No. 2464 | Cyto | 1 |
| 1214 | Seq. ID No. 2465 | Seq. ID No. 2466 | IM | |
| 1215a | Seq. ID No. 2467 | Seq. ID No. 2468 | Cyto | 0 |
| 1215b | Seq. ID No. 2469 | Seq. ID No. 2470 | Cyto | 0 |
| 1217c | Seq. ID No. 2471 | Seq. ID No. 2472 | Cyto | |
| 1218a | Seq. ID No. 2473 | Seq. ID No. 2474 | IM | 2 |
| 1218b | Seq. ID No. 2475 | Seq. ID No. 2476 | IM | 2 |
| 1219 | Seq. ID No. 2477 | Seq. ID No. 2478 | Cyto | |
| 1220 | Seq. ID No. 2479 | Seq. ID No. 2480 | Cyto | |
| 1221a | Seq. ID No. 2481 | Seq. ID No. 2482 | IM | |
| 1221b | Seq. ID No. 2483 | Seq. ID No. 2484 | IM | |
| 1223 | Seq. ID No. 2485 | Seq. ID No. 2486 | Cyto | |
| 1226 | Seq. ID No. 2495 | Seq. ID No. 2496 | Cyto | |
| 1230 | Seq. ID No. 2505 | Seq. ID No. 2506 | Cyto | 0 |
| 1233a | Seq. ID No. 2509 | Seq. ID No. 2510 | IM | |
| 1233b | Seq. ID No. 2511 | Seq. ID No. 2512 | IM | |
| 1233c | Seq. ID No. 2513 | Seq. ID No. 2514 | IM | |
| 1234c | Seq. ID No. 2519 | Seq. ID No. 2520 | IM | 6 |
| 1235 | Seq. ID No. 2521 | Seq. ID No. 2522 | Cyto | 1 |
| 1240 | Seq. ID No. 2529 | Seq. ID No. 2530 | Cyto | |
| 1244 | Seq. ID No. 2539 | Seq. ID No. 2540 | Cyto | 0 |
| 1245d | Seq. ID No. 2547 | Seq. ID No. 2548 | IM | 12 |
| 1245e | Seq. ID No. 2549 | Seq. ID No. 2550 | IM | 12 |
| 1246a | Seq. ID No. 2551 | Seq. ID No. 2552 | Cyto | |
| 1246b | Seq. ID No. 2553 | Seq. ID No. 2554 | Cyto | |
| 1250c | Seq. ID No. 2559 | Seq. ID No. 2560 | IM | 9 |
| 1250d | Seq. ID No. 2561 | Seq. ID No. 2562 | IM | 10 |
| 1250e | Seq. ID No. 2563 | Seq. ID No. 2564 | IM | 10 |
| 1251b | Seq. ID No. 2567 | Seq. ID No. 2568 | IM | 3 |
| 1253c | Seq. ID No. 2573 | Seq. ID No. 2574 | IM | 7 |
| 1253d | Seq. ID No. 2575 | Seq. ID No. 2576 | IM | 7 |
| 1256 | Seq. ID No. 2577 | Seq. ID No. 2578 | Cyto | 1 |
| 1257a | Seq. ID No. 2579 | Seq. ID No. 2580 | Cyto | |
| 1257b | Seq. ID No. 2581 | Seq. ID No. 2582 | Cyto | |
| 1258b | Seq. ID No. 2585 | Seq. ID No. 2586 | IM | 7 |
| 1258c | Seq. ID No. 2587 | Seq. ID No. 2588 | IM | 7 |
| 1258d | Seq. ID No. 2589 | Seq. ID No. 2590 | IM | 7 |
| 1263 | Seq. ID No. 2593 | Seq. ID No. 2594 | Cyto | 0 |
| 1265 | Seq. ID No. 2595 | Seq. ID No. 2596 | Cyto | 1 |
| 1267b | Seq. ID No. 2603 | Seq. ID No. 2604 | IM | 3 |
| 1268a | Seq. ID No. 2605 | Seq. ID No. 2606 | IM | 3 |
| 1269 | Seq. ID No. 2609 | Seq. ID No. 2610 | Cyto | |
| 1271 | Seq. ID No. 2613 | Seq. ID No. 2614 | Cyto | |
| 1272a | Seq. ID No. 2615 | Seq. ID No. 2616 | Cyto | |
| 1272b | Seq. ID No. 2617 | Seq. ID No. 2618 | Cyto | 0 |
| 1273 | Seq. ID No. 2619 | Seq. ID No. 2620 | Cyto | 0 |
| 1275b | Seq. ID No. 2623 | Seq. ID No. 2624 | Cyto | 1 |
| 1277 | Seq. ID No. 2625 | Seq. ID No. 2626 | Cyto | 1 |
| 1279 | Seq. ID No. 2631 | Seq. ID No. 2632 | Cyto | 0 |
| 1281 | Seq. ID No. 2633 | Seq. ID No. 2634 | Cyto | 0 |
| 1283a | Seq. ID No. 2635 | Seq. ID No. 2636 | Cyto | |
| 1283b | Seq. ID No. 2637 | Seq. ID No. 2638 | Cyto | |
| 1283c | Seq. ID No. 2639 | Seq. ID No. 2640 | Cyto | |
| 1284 | Seq. ID No. 2641 | Seq. ID No. 2642 | Cyto | |
| 1285a | Seq. ID No. 2643 | Seq. ID No. 2644 | Cyto | 0 |
| 1285b | Seq. ID No. 2645 | Seq. ID No. 2646 | Cyto | 0 |
| 1287a | Seq. ID No. 2647 | Seq. ID No. 2648 | Cyto | |
| 1287b | Seq. ID No. 2649 | Seq. ID No. 2650 | Cyto | |
| 1289 | Seq. ID No. 2653 | Seq. ID No. 2654 | IM | 1 |
| 1290 | Seq. ID No. 2655 | Seq. ID No. 2656 | Cyto | |
| 1293a | Seq. ID No. 2657 | Seq. ID No. 2658 | Cyto | |
| 1293b | Seq. ID No. 2659 | Seq. ID No. 2660 | Cyto | |
| 1294a | Seq. ID No. 2661 | Seq. ID No. 2662 | Cyto | |
| 1295 | Seq. ID No. 2663 | Seq. ID No. 2664 | Cyto | |
| 1297a | Seq. ID No. 2665 | Seq. ID No. 2666 | Cyto | |
| 1297b | Seq. ID No. 2667 | Seq. ID No. 2668 | Cyto | |
| 1300 | Seq. ID No. 2669 | Seq. ID No. 2670 | Cyto | |
| 1302 | Seq. ID No. 2671 | Seq. ID No. 2672 | Cyto | |
| 1305a | Seq. ID No. 2673 | Seq. ID No. 2674 | Cyto | |
| 1305b | Seq. ID No. 2675 | Seq. ID No. 2676 | Cyto | |
| 1305c | Seq. ID No. 2677 | Seq. ID No. 2678 | Cyto | |
| 1308d | Seq. ID No. 2685 | Seq. ID No. 2686 | IM | 4 |
| 1308e | Seq. ID No. 2687 | Seq. ID No. 2688 | IM | 4 |
| 1311 | Seq. ID No. 2689 | Seq. ID No. 2690 | Cyto | |
| 1320c | Seq. ID No. 2709 | Seq. ID No. 2710 | IM | 5 |
| 1321 | Seq. ID No. 2711 | Seq. ID No. 2712 | Cyto | |
| 1322a | Seq. ID No. 2713 | Seq. ID No. 2714 | Cyto | |
| 1322b | Seq. ID No. 2715 | Seq. ID No. 2716 | Cyto | |
| 1324 | Seq. ID No. 2719 | Seq. ID No. 2720 | Cyto | |
| 1326a | Seq. ID No. 2721 | Seq. ID No. 2722 | Cyto | |
| 1326b | Seq. ID No. 2723 | Seq. ID No. 2724 | Cyto | |
| 1326c | Seq. ID No. 2725 | Seq. ID No. 2726 | Cyto | |
| 1333 | Seq. ID No. 2727 | Seq. ID No. 2728 | Cyto | 0 |
| 1335b | Seq. ID No. 2735 | Seq. ID No. 2736 | IM | 3 |
| 1337 | Seq. ID No. 2737 | Seq. ID No. 2738 | Cyto | |
| 1338 | Seq. ID No. 2739 | Seq. ID No. 2740 | IM | 1 |
| 1340a | Seq. ID No. 2741 | Seq. ID No. 2742 | IM | 1 |
| 1340b | Seq. ID No. 2743 | Seq. ID No. 2744 | IM | 1 |
| 1341a | Seq. ID No. 2745 | Seq. ID No. 2746 | IM | 3 |
| 1341c | Seq. ID No. 2747 | Seq. ID No. 2748 | IM | 4 |
| 1344a | Seq. ID No. 2749 | Seq. ID No. 2750 | Cyto | 0 |
| 1344b | Seq. ID No. 2751 | Seq. ID No. 2752 | Cyto | 0 |
| 1348 | Seq. ID No. 2753 | Seq. ID No. 2754 | IM | 5 |
| 1349a | Seq. ID No. 2755 | Seq. ID No. 2756 | IM | 1 |
| 1349b | Seq. ID No. 2757 | Seq. ID No. 2758 | IM | 1 |
| 1349c | Seq. ID No. 2759 | Seq. ID No. 2760 | IM | 1 |
| 1350a | Seq. ID No. 2761 | Seq. ID No. 2762 | Cyto | |
| 1350b | Seq. ID No. 2763 | Seq. ID No. 2764 | Cyto | |
| 1353 | Seq. ID No. 2765 | Seq. ID No. 2766 | Cyto | 2 |
| 1355 | Seq. ID No. 2767 | Seq. ID No. 2768 | Cyto | |
| 1358a | Seq. ID No. 2769 | Seq. ID No. 2770 | Cyto | 0 |
| 1358b | Seq. ID No. 2771 | Seq. ID No. 2772 | Cyto | 0 |
| 1359 | Seq. ID No. 2773 | Seq. ID No. 2774 | Cyto | 1 |
| 1361 | Seq. ID No. 2775 | Seq. ID No. 2776 | Cyto | |
| 1364a | Seq. ID No. 2777 | Seq. ID No. 2778 | Cyto | |
| 1364b | Seq. ID No. 2779 | Seq. ID No. 2780 | Cyto | |
| 1364c | Seq. ID No. 2781 | Seq. ID No. 2782 | Cyto | |
| 1366b | Seq. ID No. 2787 | Seq. ID No. 2788 | IM | 1 |
| 1367 | Seq. ID No. 2789 | Seq. ID No. 2790 | Cyto | 1 |
| 1368 | Seq. ID No. 2791 | Seq. ID No. 2792 | Cyto | 0 |
| 1371 | Seq. ID No. 2793 | Seq. ID No. 2794 | Cyto | |
| 1373a | Seq. ID No. 2795 | Seq. ID No. 2796 | Cyto | |
| 1373b | Seq. ID No. 2797 | Seq. ID No. 2798 | Cyto | |
| 1374 | Seq. ID No. 2799 | Seq. ID No. 2800 | IM | |
| 1377 | Seq. ID No. 2803 | Seq. ID No. 2804 | IM | 0 |
| 1378 | Seq. ID No. 2805 | Seq. ID No. 2806 | IM | 0 |
| 1379 | Seq. ID No. 2807 | Seq. ID No. 2808 | IM | 1 |
| 1381a | Seq. ID No. 2811 | Seq. ID No. 2812 | IM | 1 |
| 1383 | Seq. ID No. 2817 | Seq. ID No. 2818 | Cyto | |
| 1386a | Seq. ID No. 2825 | Seq. ID No. 2826 | IM | 1 |
| 1386b | Seq. ID No. 2827 | Seq. ID No. 2828 | IM | 1 |
| 1387a | Seq. ID No. 2829 | Seq. ID No. 2830 | OM | 1 |
| 1394 | Seq. ID No. 2843 | Seq. ID No. 2844 | Cyto | 1 |
| 1396 | Seq. ID No. 2847 | Seq. ID No. 2848 | Cyto | |
| 1397 | Seq. ID No. 2849 | Seq. ID No. 2850 | Peri | |
| 1398 | Seq. ID No. 2851 | Seq. ID No. 2852 | Cyto | |
| 1400 | Seq. ID No. 2857 | Seq. ID No. 2858 | IM | 1 |
| 1405 | Seq. ID No. 2863 | Seq. ID No. 2864 | IM | 2 |
| 1407b | Seq. ID No. 2865 | Seq. ID No. 2866 | IM | 1 |
| 1408 | Seq. ID No. 2867 | Seq. ID No. 2868 | IM | 5 |
| 1409 | Seq. ID No. 2869 | Seq. ID No. 2870 | IM | 1 |
| 1410 | Seq. ID No. 2871 | Seq. ID No. 2872 | Cyto | |
| 1411b | Seq. ID No. 2875 | Seq. ID No. 2876 | IM | 4 |
| 1411c | Seq. ID No. 2877 | Seq. ID No. 2878 | IM | 6 |
| 1412a | Seq. ID No. 2879 | Seq. ID No. 2880 | IM | 8 |
| 1414a | Seq. ID No. 2883 | Seq. ID No. 2884 | IM | 0 |
| 1414b | Seq. ID No. 2885 | Seq. ID No. 2886 | IM | 0 |
| 1414c | Seq. ID No. 2887 | Seq. ID No. 2888 | IM | 0 |
| 1415 | Seq. ID No. 2889 | Seq. ID No. 2890 | Cyto | |
| 1416 | Seq. ID No. 2891 | Seq. ID No. 2892 | Cyto | |
| 1419 | Seq. ID No. 2895 | Seq. ID No. 2896 | IM | |
| 1420a | Seq. ID No. 2897 | Seq. ID No. 2898 | IM | 0 |
| 1420b | Seq. ID No. 2899 | Seq. ID No. 2900 | IM | 0 |
| 1422 | Seq. ID No. 2901 | Seq. ID No. 2902 | Cyto | 0 |
| 1423 | Seq. ID No. 2903 | Seq. ID No. 2904 | Cyto | |
| 1426b | Seq. ID No. 2907 | Seq. ID No. 2908 | IM | 6 |
| 1427a | Seq. ID No. 2909 | Seq. ID No. 2910 | Cyto | |
| 1427b | Seq. ID No. 2911 | Seq. ID No. 2912 | Cyto | |
| 1428 | Seq. ID No. 2913 | Seq. ID No. 2914 | Cyto | |
| 1429a | Seq. ID No. 2915 | Seq. ID No. 2916 | Cyto | |
| 1429b | Seq. ID No. 2917 | Seq. ID No. 2918 | IM | |
| 1431 | Seq. ID No. 2919 | Seq. ID No. 2920 | Cyto | 0 |
| 1432 | Seq. ID No. 2921 | Seq. ID No. 2922 | Cyto | 0 |
| 1433 | Seq. ID No. 2923 | Seq. ID No. 2924 | Cyto | 0 |
| 1434a | Seq. ID No. 2925 | Seq. ID No. 2926 | Cyto | 1 |
| 1434b | Seq. ID No. 2927 | Seq. ID No. 2928 | Cyto | 1 |
| 1437a | Seq. ID No. 2929 | Seq. ID No. 2930 | Cyto | 0 |
| 1437b | Seq. ID No. 2931 | Seq. ID No. 2932 | Cyto | 0 |
| 1437c | Seq. ID No. 2933 | Seq. ID No. 2934 | Cyto | 0 |
| 1439d | Seq. ID No. 2943 | Seq. ID No. 2944 | IM | 7 |
| 1439e | Seq. ID No. 2945 | Seq. ID No. 2946 | IM | 7 |
| 1440d | Seq. ID No. 2953 | Seq. ID No. 2954 | IM | 12 |
| 1442a | Seq. ID No. 2955 | Seq. ID No. 2956 | Cyto | |
| 1442b | Seq. ID No. 2957 | Seq. ID No. 2958 | Cyto | |
| 1445a | Seq. ID No. 2959 | Seq. ID No. 2960 | Cyto | |
| 1445b | Seq. ID No. 2961 | Seq. ID No. 2962 | Cyto | |
| 1445c | Seq. ID No. 2963 | Seq. ID No. 2964 | Cyto | |
| 1446a | Seq. ID No. 2965 | Seq. ID No. 2966 | IM | 2 |
| 1446b | Seq. ID No. 2967 | Seq. ID No. 2968 | IM | 2 |
| 1447b | Seq. ID No. 2971 | Seq. ID No. 2972 | IM | 11 |
| 1451 | Seq. ID No. 2979 | Seq. ID No. 2980 | Cyto | |
| 1453 | Seq. ID No. 2985 | Seq. ID No. 2986 | IM | 5 |
| 1455a | Seq. ID No. 2989 | Seq. ID No. 2990 | Cyto | |
| 1456 | Seq. ID No. 2993 | Seq. ID No. 2994 | Cyto | |
| 1457c | Seq. ID No. 2999 | Seq. ID No. 3000 | IM | 8 |
| 1459 | Seq. ID No. 3001 | Seq. ID No. 3002 | IM | 1 |
| 1462a | Seq. ID No. 3003 | Seq. ID No. 3004 | IM | 5 |
| 1466b | Seq. ID No. 3015 | Seq. ID No. 3016 | IM | 2 |
| 1466c | Seq. ID No. 3017 | Seq. ID No. 3018 | IM | 2 |
| 1466d | Seq. ID No. 3019 | Seq. ID No. 3020 | IM | 2 |
| 1467 | Seq. ID No. 3021 | Seq. ID No. 3022 | Cyto | 2 |
| 1470c | Seq. ID No. 3033 | Seq. ID No. 3034 | IM | 8 |
| 1471 | Seq. ID No. 3035 | Seq. ID No. 3036 | IM | 6 |
| 1472b | Seq. ID No. 3037 | Seq. ID No. 3038 | IM | |
| 1472c | Seq. ID No. 3039 | Seq. ID No. 3040 | Cyto | |
| 1474 | Seq. ID No. 3041 | Seq. ID No. 3042 | Cyto | |
| 1476 | Seq. ID No. 3045 | Seq. ID No. 3046 | IM | 5 |
| 1477a | Seq. ID No. 3047 | Seq. ID No. 3048 | Cyto | 0 |
| 1477b | Seq. ID No. 3049 | Seq. ID No. 3050 | Cyto | 0 |
| 1479a | Seq. ID No. 3051 | Seq. ID No. 3052 | Cyto | |
| 1479b | Seq. ID No. 3053 | Seq. ID No. 3054 | Cyto | |
| 1482a | Seq. ID No. 3057 | Seq. ID No. 3058 | Cyto | 0 |
| 1482b | Seq. ID No. 3059 | Seq. ID No. 3060 | Cyto | 0 |
| 1483 | Seq. ID No. 3061 | Seq. ID No. 3062 | Cyto | |
| 1484b | Seq. ID No. 3065 | Seq. ID No. 3066 | IM | 4 |
| 1484c | Seq. ID No. 3067 | Seq. ID No. 3068 | IM | 4 |
| 1486 | Seq. ID No. 3069 | Seq. ID No. 3070 | Cyto | |
| 1488d | Seq. ID No. 3079 | Seq. ID No. 3080 | IM | 4 |
| 1488e | Seq. ID No. 3081 | Seq. ID No. 3082 | IM | 4 |
| 1488f | Seq. ID No. 3083 | Seq. ID No. 3084 | IM | 4 |
| 1488g | Seq. ID No. 3085 | Seq. ID No. 3086 | IM | 4 |
| 1489 | Seq. ID No. 3087 | Seq. ID No. 3088 | Cyto | |
| 1490 | Seq. ID No. 3089 | Seq. ID No. 3090 | Cyto | |
| 1494 | Seq. ID No. 3091 | Seq. ID No. 3092 | Cyto | |
| 1497 | Seq. ID No. 3093 | Seq. ID No. 3094 | Cyto | 0 |
| 1498a | Seq. ID No. 3095 | Seq. ID No. 3096 | Cyto | |
| 1498b | Seq. ID No. 3097 | Seq. ID No. 3098 | Cyto | |
| 1498c | Seq. ID No. 3099 | Seq. ID No. 3100 | Cyto | |
| 1498d | Seq. ID No. 3101 | Seq. ID No. 3102 | Cyto | |
| 1499 | Seq. ID No. 3103 | Seq. ID No. 3104 | Cyto | |
| 1502a | Seq. ID No. 3105 | Seq. ID No. 3106 | Cyto | |
| 1502b | Seq. ID No. 3107 | Seq. ID No. 3108 | Cyto | |
| 1503a | Seq. ID No. 3109 | Seq. ID No. 3110 | IM | 4 |
| 1503b | Seq. ID No. 3111 | Seq. ID No. 3112 | IM | 4 |
| 1506a | Seq. ID No. 3113 | Seq. ID No. 3114 | Cyto | 0 |
| 1506b | Seq. ID No. 3115 | Seq. ID No. 3116 | Cyto | 0 |
| 1506c | Seq. ID No. 3117 | Seq. ID No. 3118 | Cyto | 0 |
| 1508 | Seq. ID No. 3125 | Seq. ID No. 3126 | Cyto | 0 |
| 1510a | Seq. ID No. 3127 | Seq. ID No. 3128 | IM | 0 |
| 1510b | Seq. ID No. 3129 | Seq. ID No. 3130 | IM | 0 |
| 1510c | Seq. ID No. 3131 | Seq. ID No. 3132 | IM | 0 |
| 1510d | Seq. ID No. 3133 | Seq. ID No. 3134 | IM | 0 |
| 1511 | Seq. ID No. 3135 | Seq. ID No. 3136 | IM | 0 |
| 1512 | Seq. ID No. 3137 | Seq. ID No. 3138 | Cyto | |
| 1513a | Seq. ID No. 3139 | Seq. ID No. 3140 | Cyto | |
| 1513b | Seq. ID No. 3141 | Seq. ID No. 3142 | Cyto | |
| 1516 | Seq. ID No. 3143 | Seq. ID No. 3144 | Cyto | |
| 1517b | Seq. ID No. 3147 | Seq. ID No. 3148 | IM | 3 |
| 1517c | Seq. ID No. 3149 | Seq. ID No. 3150 | IM | 3 |
| 1518a | Seq. ID No. 3151 | Seq. ID No. 3152 | Cyto | 0 |
| 1518b | Seq. ID No. 3153 | Seq. ID No. 3154 | Cyto | 0 |
| 1523 | Seq. ID No. 3159 | Seq. ID No. 3160 | Cyto | 0 |
| 1528 | Seq. ID No. 3161 | Seq. ID No. 3162 | Cyto | |
| 1533a | Seq. ID No. 3167 | Seq. ID No. 3168 | IM | 0 |
| 1534a | Seq. ID No. 3169 | Seq. ID No. 3170 | Cyto | 0 |
| 1534b | Seq. ID No. 3171 | Seq. ID No. 3172 | Cyto | 0 |
| 1536a | Seq. ID No. 3175 | Seq. ID No. 3176 | Cyto | |
| 1536b | Seq. ID No. 3177 | Seq. ID No. 3178 | Cyto | 0 |
| 1537 | Seq. ID No. 3179 | Seq. ID No. 3180 | Cyto | |
| 1542 | Seq. ID No. 3181 | Seq. ID No. 3182 | IM | 0 |
| 1544 | Seq. ID No. 3185 | Seq. ID No. 3186 | Cyto | 1 |
| 1547a | Seq. ID No. 3187 | Seq. ID No. 3188 | Cyto | |
| 1547b | Seq. ID No. 3189 | Seq. ID No. 3190 | Cyto | |
| 1548 | Seq. ID No. 3191 | Seq. ID No. 3192 | Cyto | |
| 1553a | Seq. ID No. 3195 | Seq. ID No. 3196 | Cyto | |
| 1553b | Seq. ID No. 3197 | Seq. ID No. 3198 | Cyto | |
| 1554a | Seq. ID No. 3199 | Seq. ID No. 3200 | IM | 1 |
| 1554c | Seq. ID No. 3203 | Seq. ID No. 3204 | IM | 3 |
| 1555 | Seq. ID No. 3205 | Seq. ID No. 3206 | IM | 1 |
| 1556a | Seq. ID No. 3207 | Seq. ID No. 3208 | Cyto | |
| 1556b | Seq. ID No. 3209 | Seq. ID No. 3210 | Cyto | |
| 1556c | Seq. ID No. 3211 | Seq. ID No. 3212 | Cyto | |
| 1559b | Seq. ID No. 3221 | Seq. ID No. 3222 | IM | 7 |
| 1563 | Seq. ID No. 3223 | Seq. ID No. 3224 | Cyto | |
| 1564a | Seq. ID No. 3225 | Seq. ID No. 3226 | Cyto | 1 |
| 1564b | Seq. ID No. 3227 | Seq. ID No. 3228 | Cyto | 1 |
| 1565 | Seq. ID No. 3229 | Seq. ID No. 3230 | Cyto | |
| 1567 | Seq. ID No. 3231 | Seq. ID No. 3232 | Cyto | |
| 1568b | Seq. ID No. 3233 | Seq. ID No. 3234 | IM | 1 |
| 1569 | Seq. ID No. 3235 | Seq. ID No. 3236 | IM | 2 |
| 1570 | Seq. ID No. 3237 | Seq. ID No. 3238 | Peri | 2 |
| 1571 | Seq. ID No. 3239 | Seq. ID No. 3240 | IM | 5 |
| 1572d | Seq. ID No. 3247 | Seq. ID No. 3248 | IM | 11 |
| 1573 | Seq. ID No. 3251 | Seq. ID No. 3252 | Cyto | |
| 1576 | Seq. ID No. 3253 | Seq. ID No. 3254 | Cyto | |
| 1578 | Seq. ID No. 3259 | Seq. ID No. 3260 | IM | 2 |
| 1581 | Seq. ID No. 3265 | Seq. ID No. 3266 | IM | 1 |
| 1582a | Seq. ID No. 3267 | Seq. ID No. 3268 | IM | |
| 1582c | Seq. ID No. 3271 | Seq. ID No. 3272 | IM | 1 |
| 1584 | Seq. ID No. 3273 | Seq. ID No. 3274 | Peri | 1 |
| 1587a | Seq. ID No. 3279 | Seq. ID No. 3280 | Cyto | |
| 1587b | Seq. ID No. 3281 | Seq. ID No. 3282 | Cyto | |
| 1587c | Seq. ID No. 3283 | Seq. ID No. 3284 | Cyto | |
| 1588 | Seq. ID No. 3285 | Seq. ID No. 3286 | IM | 0 |
| 1590 | Seq. ID No. 3287 | Seq. ID No. 3288 | Cyto | |
| 1591 | Seq. ID No. 3289 | Seq. ID No. 3290 | Cyto | |
| 1592a | Seq. ID No. 3291 | Seq. ID No. 3292 | Cyto | |
| 1592b | Seq. ID No. 3293 | Seq. ID No. 3294 | Cyto | |
| 1593 | Seq. ID No. 3295 | Seq. ID No. 3296 | Cyto | |
| 1594b | Seq. ID No. 3299 | Seq. ID No. 3300 | IM | 1 |
| 1595a | Seq. ID No. 3301 | Seq. ID No. 3302 | Cyto | 0 |
| 1595b | Seq. ID No. 3303 | Seq. ID No. 3304 | Cyto | 0 |
| 1598a | Seq. ID No. 3305 | Seq. ID No. 3306 | Cyto | 0 |
| 1598b | Seq. ID No. 3307 | Seq. ID No. 3308 | Cyto | 0 |
| 1600a | Seq. ID No. 3309 | Seq. ID No. 3310 | Cyto | 1 |
| 1600b | Seq. ID No. 3311 | Seq. ID No. 3312 | Cyto | 1 |
| 1600c | Seq. ID No. 3313 | Seq. ID No. 3314 | Cyto | 1 |
| 1602 | Seq. ID No. 3315 | Seq. ID No. 3316 | Cyto | |
| 1606b | Seq. ID No. 3319 | Seq. ID No. 3320 | IM | 2 |
| 1607a | Seq. ID No. 3321 | Seq. ID No. 3322 | IM | 5 |
| 1610a | Seq. ID No. 3327 | Seq. ID No. 3328 | Cyto | |
| 1610b | Seq. ID No. 3329 | Seq. ID No. 3330 | Cyto | |
| 1616a | Seq. ID No. 3333 | Seq. ID No. 3334 | IM | 1 |
| 1622a | Seq. ID No. 3341 | Seq. ID No. 3342 | Cyto | 0 |
| 1623a | Seq. ID No. 3343 | Seq. ID No. 3344 | Cyto | |
| 1623b | Seq. ID No. 3345 | Seq. ID No. 3346 | Cyto | |
| 1623c | Seq. ID No. 3347 | Seq. ID No. 3348 | Cyto | |
| 1628a | Seq. ID No. 3361 | Seq. ID No. 3362 | Cyto | |
| 1628b | Seq. ID No. 3363 | Seq. ID No. 3364 | Cyto | |
| 1632a | Seq. ID No. 3365 | Seq. ID No. 3366 | Cyto | 0 |
| 1632b | Seq. ID No. 3367 | Seq. ID No. 3368 | Cyto | 0 |
| 1633a | Seq. ID No. 3369 | Seq. ID No. 3370 | IM | 2 |
| 1633b | Seq. ID No. 3371 | Seq. ID No. 3372 | IM | 2 |
| 1634a | Seq. ID No. 3373 | Seq. ID No. 3374 | Cyto | |
| 1639b | Seq. ID No. 3379 | Seq. ID No. 3380 | Cyto | |
| 1642a | Seq. ID No. 3381 | Seq. ID No. 3382 | Cyto | 1 |
| 1642b | Seq. ID No. 3383 | Seq. ID No. 3384 | Cyto | 1 |
| 1643a | Seq. ID No. 3385 | Seq. ID No. 3386 | Cyto | |
| 1643b | Seq. ID No. 3387 | Seq. ID No. 3388 | Cyto | |
| 1646c | Seq. ID No. 3393 | Seq. ID No. 3394 | IM | 11 |
| 1646e | Seq. ID No. 3397 | Seq. ID No. 3398 | IM | 13 |
| 1646f | Seq. ID No. 3399 | Seq. ID No. 3400 | IM | 13 |
| 1647 | Seq. ID No. 3401 | Seq. ID No. 3402 | Cyto | |
| 1648b | Seq. ID No. 3403 | Seq. ID No. 3404 | IM | 3 |
| 1649b | Seq. ID No. 3407 | Seq. ID No. 3408 | Cyto | 2 |
| 1649c | Seq. ID No. 3409 | Seq. ID No. 3410 | Cyto | 2 |
| 1652a | Seq. ID No. 3411 | Seq. ID No. 3412 | Cyto | |
| 1652b | Seq. ID No. 3413 | Seq. ID No. 3414 | Cyto | |
| 1656b | Seq. ID No. 3417 | Seq. ID No. 3418 | Cyto | |
| 1657 | Seq. ID No. 3419 | Seq. ID No. 3420 | Cyto | |
| 1659b | Seq. ID No. 3423 | Seq. ID No. 3424 | IM | 9 |
| 1662a | Seq. ID No. 3429 | Seq. ID No. 3430 | Cyto | 1 |
| 1662b | Seq. ID No. 3431 | Seq. ID No. 3432 | Cyto | 1 |
| 1664 | Seq. ID No. 3435 | Seq. ID No. 3436 | IM | 0 |
| 1665a | Seq. ID No. 3437 | Seq. ID No. 3438 | Cyto | 1 |
| 1668 | Seq. ID No. 3439 | Seq. ID No. 3440 | IM | 1 |
| 1670a | Seq. ID No. 3443 | Seq. ID No. 3444 | Cyto | |
| 1670b | Seq. ID No. 3445 | Seq. ID No. 3446 | Cyto | |
| 1670c | Seq. ID No. 3447 | Seq. ID No. 3448 | IM | |
| 1673f | Seq. ID No. 3459 | Seq. ID No. 3460 | IM | 14 |
| 1674 | Seq. ID No. 3461 | Seq. ID No. 3462 | OM | 1 |
| 1675b | Seq. ID No. 3465 | Seq. ID No. 3466 | Cyto | 1 |
| 1675c | Seq. ID No. 3467 | Seq. ID No. 3468 | Cyto | 1 |
| 1676 | Seq. ID No. 3469 | Seq. ID No. 3470 | Cyto | 1 |
| 1677 | Seq. ID No. 3471 | Seq. ID No. 3472 | Cyto | 0 |
| 1679 | Seq. ID No. 3473 | Seq. ID No. 3474 | IM | 1 |
| 1680 | Seq. ID No. 3475 | Seq. ID No. 3476 | IM | |
| 1682a | Seq. ID No. 3477 | Seq. ID No. 3478 | Cyto | |
| 1682b | Seq. ID No. 3479 | Seq. ID No. 3480 | Cyto | |
| 1683b | Seq. ID No. 3483 | Seq. ID No. 3484 | IM | 6 |
| 1684 | Seq. ID No. 3485 | Seq. ID No. 3486 | Cyto | |
| 1685 | Seq. ID No. 3487 | Seq. ID No. 3488 | Cyto | 0 |
| 1687 | Seq. ID No. 3489 | Seq. ID No. 3490 | Cyto | 0 |
| 1688c | Seq. ID No. 3495 | Seq. ID No. 3496 | IM | 11 |
| 1689a | Seq. ID No. 3497 | Seq. ID No. 3498 | IM | |
| 1689b | Seq. ID No. 3499 | Seq. ID No. 3500 | IM | |
| 1689c | Seq. ID No. 3501 | Seq. ID No. 3502 | Cyto | |
| 1691 | Seq. ID No. 3507 | Seq. ID No. 3508 | IM | 9 |
| 1692 | Seq. ID No. 3509 | Seq. ID No. 3510 | Cyto | |
| 1693 | Seq. ID No. 3511 | Seq. ID No. 3512 | IM | |
| 1694a | Seq. ID No. 3513 | Seq. ID No. 3514 | Cyto | |
| 1694b | Seq. ID No. 3515 | Seq. ID No. 3516 | Cyto | |
| 1696 | Seq. ID No. 3521 | Seq. ID No. 3522 | Peri | 1 |
| 1697a | Seq. ID No. 3523 | Seq. ID No. 3524 | IM | 7 |
| 1697b | Seq. ID No. 3525 | Seq. ID No. 3526 | IM | 9 |
| 1699b | Seq. ID No. 3529 | Seq. ID No. 3530 | IM | 10 |
| 1700b | Seq. ID No. 3533 | Seq. ID No. 3534 | IM | 4 |
| 1701a | Seq. ID No. 3535 | Seq. ID No. 3536 | Cyto | 0 |
| 1701b | Seq. ID No. 3537 | Seq. ID No. 3538 | Cyto | 0 |
| 1704a | Seq. ID No. 3541 | Seq. ID No. 3542 | IM | 0 |
| 1704b | Seq. ID No. 3543 | Seq. ID No. 3544 | Cyto | 0 |
| 1705a | Seq. ID No. 3545 | Seq. ID No. 3546 | Cyto | |
| 1705b | Seq. ID No. 3547 | Seq. ID No. 3548 | Cyto | |
| 1706 | Seq. ID No. 3549 | Seq. ID No. 3550 | Cyto | |
| 1707a | Seq. ID No. 3551 | Seq. ID No. 3552 | Cyto | |
| 1707b | Seq. ID No. 3553 | Seq. ID No. 3554 | Cyto | |
| 1708 | Seq. ID No. 3555 | Seq. ID No. 3556 | Cyto | 0 |
| 1709 | Seq. ID No. 3557 | Seq. ID No. 3558 | Cyto | |
| 1710a | Seq. ID No. 3559 | Seq. ID No. 3560 | Peri | |
| 1712a | Seq. ID No. 3563 | Seq. ID No. 3564 | Cyto | |
| 1712b | Seq. ID No. 3565 | Seq. ID No. 3566 | Cyto | |
| 1715a | Seq. ID No. 3567 | Seq. ID No. 3568 | Cyto | |
| 1715b | Seq. ID No. 3569 | Seq. ID No. 3570 | Cyto | |
| 1718a | Seq. ID No. 3571 | Seq. ID No. 3572 | Cyto | |
| 1722 | Seq. ID No. 3575 | Seq. ID No. 3576 | IM | 1 |
| 1723a | Seq. ID No. 3577 | Seq. ID No. 3578 | Cyto | 0 |
| 1723b | Seq. ID No. 3579 | Seq. ID No. 3580 | Cyto | 0 |
| 1727 | Seq. ID No. 3583 | Seq. ID No. 3584 | Cyto | 0 |
| 1728 | Seq. ID No. 3585 | Seq. ID No. 3586 | Cyto | |
| 1733 | Seq. ID No. 3589 | Seq. ID No. 3590 | Cyto | |
| 1734 | Seq. ID No. 3591 | Seq. ID No. 3592 | Cyto | |
| 1737a | Seq. ID No. 3597 | Seq. ID No. 3598 | Cyto | |
| 1737b | Seq. ID No. 3599 | Seq. ID No. 3600 | Cyto | |
| 1738a | Seq. ID No. 3601 | Seq. ID No. 3602 | Cyto | |
| 1738b | Seq. ID No. 3603 | Seq. ID No. 3604 | Cyto | 0 |
| 1738c | Seq. ID No. 3605 | Seq. ID No. 3606 | Cyto | 0 |
| 1739 | Seq. ID No. 3607 | Seq. ID No. 3608 | Cyto | |
| 1741 | Seq. ID No. 3609 | Seq. ID No. 3610 | Cyto | |
| 1744a | Seq. ID No. 3613 | Seq. ID No. 3614 | Cyto | |
| 1744b | Seq. ID No. 3615 | Seq. ID No. 3616 | Cyto | |
| 1749b | Seq. ID No. 3623 | Seq. ID No. 3624 | IM | 4 |
| 1752a | Seq. ID No. 3627 | Seq. ID No. 3628 | Cyto | |
| 1753 | Seq. ID No. 3631 | Seq. ID No. 3632 | Cyto | |
| 1756a | Seq. ID No. 3633 | Seq. ID No. 3634 | Cyto | |
| 1757a | Seq. ID No. 3637 | Seq. ID No. 3638 | IM | 2 |
| 1757b | Seq. ID No. 3639 | Seq. ID No. 3640 | IM | 2 |
| 1759 | Seq. ID No. 3641 | Seq. ID No. 3642 | Cyto | |
| 1760 | Seq. ID No. 3643 | Seq. ID No. 3644 | Cyto | |
| 1762 | Seq. ID No. 3645 | Seq. ID No. 3646 | Cyto | |
| 1764a | Seq. ID No. 3647 | Seq. ID No. 3648 | IM | |
| 1764b | Seq. ID No. 3649 | Seq. ID No. 3650 | IM | |
| 1764c | Seq. ID No. 3651 | Seq. ID No. 3652 | IM | |
| 1765 | Seq. ID No. 3653 | Seq. ID No. 3654 | Cyto | |
| 1766 | Seq. ID No. 3655 | Seq. ID No. 3656 | IM | 0 |
| 1767a | Seq. ID No. 3657 | Seq. ID No. 3658 | Cyto | |
| 1767b | Seq. ID No. 3659 | Seq. ID No. 3660 | Cyto | |
| 1769a | Seq. ID No. 3661 | Seq. ID No. 3662 | IM | 5 |
| 1769b | Seq. ID No. 3663 | Seq. ID No. 3664 | IM | 7 |
| 1771 | Seq. ID No. 3665 | Seq. ID No. 3666 | Cyto | |
| 1773 | Seq. ID No. 3667 | Seq. ID No. 3668 | Cyto | |
| 1774 | Seq. ID No. 3669 | Seq. ID No. 3670 | IM | |
| 1775a | Seq. ID No. 3671 | Seq. ID No. 3672 | Cyto | |
| 1775b | Seq. ID No. 3673 | Seq. ID No. 3674 | Cyto | |
| 1775c | Seq. ID No. 3675 | Seq. ID No. 3676 | Cyto | |
| 1780 | Seq. ID No. 3681 | Seq. ID No. 3682 | IM | 0 |
| 1783a | Seq. ID No. 3683 | Seq. ID No. 3684 | IM | 1 |
| 1783b | Seq. ID No. 3685 | Seq. ID No. 3686 | IM | 1 |
| 1784a | Seq. ID No. 3687 | Seq. ID No. 3688 | IM | |
| 1788a | Seq. ID No. 3691 | Seq. ID No. 3692 | Cyto | |
| 1788b | Seq. ID No. 3693 | Seq. ID No. 3694 | IM | |
| 1788c | Seq. ID No. 3695 | Seq. ID No. 3696 | Cyto | |
| 1789a | Seq. ID No. 3697 | Seq. ID No. 3698 | Cyto | |
| 1789b | Seq. ID No. 3699 | Seq. ID No. 3700 | Cyto | |
| 1790a | Seq. ID No. 3701 | Seq. ID No. 3702 | Cyto | 2 |
| 1790b | Seq. ID No. 3703 | Seq. ID No. 3704 | Cyto | 3 |
| 1792a | Seq. ID No. 3705 | Seq. ID No. 3706 | Peri | 0 |
| 1792b | Seq. ID No. 3707 | Seq. ID No. 3708 | Cyto | 0 |
| 1792c | Seq. ID No. 3709 | Seq. ID No. 3710 | Cyto | 0 |
| 1793a | Seq. ID No. 3711 | Seq. ID No. 3712 | IM | |
| 1793b | Seq. ID No. 3713 | Seq. ID No. 3714 | IM | |
| 1793c | Seq. ID No. 3715 | Seq. ID No. 3716 | IM | |
| 1797a | Seq. ID No. 3719 | Seq. ID No. 3720 | Cyto | |
| 1797b | Seq. ID No. 3721 | Seq. ID No. 3722 | Cyto | |
| 1798b | Seq. ID No. 3725 | Seq. ID No. 3726 | IM | 4 |
| 1800 | Seq. ID No. 3729 | Seq. ID No. 3730 | IM | 1 |
| 1802a | Seq. ID No. 3731 | Seq. ID No. 3732 | IM | 2 |
| 1802b | Seq. ID No. 3733 | Seq. ID No. 3734 | IM | 2 |
| 1803 | Seq. ID No. 3735 | Seq. ID No. 3736 | IM | 3 |
| 1804a | Seq. ID No. 3737 | Seq. ID No. 3738 | IM | 2 |
| 1804b | Seq. ID No. 3739 | Seq. ID No. 3740 | IM | 2 |
| 1806a | Seq. ID No. 3743 | Seq. ID No. 3744 | IM | 5 |
| 1806c | Seq. ID No. 3747 | Seq. ID No. 3748 | IM | 5 |
| 1807a | Seq. ID No. 3749 | Seq. ID No. 3750 | IM | 2 |
| 1807b | Seq. ID No. 3751 | Seq. ID No. 3752 | IM | 2 |
| 1808d | Seq. ID No. 3759 | Seq. ID No. 3760 | IM | 15 |
| 1809d | Seq. ID No. 3767 | Seq. ID No. 3768 | IM | 12 |
| 1809e | Seq. ID No. 3769 | Seq. ID No. 3770 | IM | 14 |
| 1809f | Seq. ID No. 3771 | Seq. ID No. 3772 | IM | 14 |
| 1810a | Seq. ID No. 3773 | Seq. ID No. 3774 | Cyto | |
| 1810b | Seq. ID No. 3775 | Seq. ID No. 3776 | Cyto | |
| 1811a | Seq. ID No. 3777 | Seq. ID No. 3778 | Cyto | |
| 1811b | Seq. ID No. 3779 | Seq. ID No. 3780 | Cyto | |
| 1813 | Seq. ID No. 3785 | Seq. ID No. 3786 | IM | 1 |
| 1815a | Seq. ID No. 3787 | Seq. ID No. 3788 | Cyto | |
| 1815b | Seq. ID No. 3789 | Seq. ID No. 3790 | Cyto | |
| 1815c | Seq. ID No. 3791 | Seq. ID No. 3792 | Cyto | |
| 1816b | Seq. ID No. 3795 | Seq. ID No. 3796 | IM | 1 |
| 1816c | Seq. ID No. 3797 | Seq. ID No. 3798 | IM | 1 |
| 1819a | Seq. ID No. 3801 | Seq. ID No. 3802 | IM | 0 |
| 1819b | Seq. ID No. 3803 | Seq. ID No. 3804 | IM | 0 |
| 1821 | Seq. ID No. 3805 | Seq. ID No. 3806 | Cyto | 0 |
| 1823a | Seq. ID No. 3809 | Seq. ID No. 3810 | Cyto | |
| 1823b | Seq. ID No. 3811 | Seq. ID No. 3812 | Cyto | |
| 1828 | Seq. ID No. 3819 | Seq. ID No. 3820 | Cyto | |
| 1830a | Seq. ID No. 3821 | Seq. ID No. 3822 | IM | 6 |
| 1830b | Seq. ID No. 3823 | Seq. ID No. 3824 | IM | 7 |
| 1833 | Seq. ID No. 3825 | Seq. ID No. 3826 | Cyto | |
| 1834 | Seq. ID No. 3827 | Seq. ID No. 3828 | Cyto | |
| 1835 | Seq. ID No. 3829 | Seq. ID No. 3830 | Cyto | |
| 1836a | Seq. ID No. 3831 | Seq. ID No. 3832 | IM | 1 |
| 1836b | Seq. ID No. 3833 | Seq. ID No. 3834 | IM | 1 |
| 1837 | Seq. ID No. 3835 | Seq. ID No. 3836 | IM | |
| 1838c | Seq. ID No. 3841 | Seq. ID No. 3842 | IM | 6 |
| 1838d | Seq. ID No. 3843 | Seq. ID No. 3844 | IM | 6 |
| 1839a | Seq. ID No. 3845 | Seq. ID No. 3846 | Cyto | 0 |
| 1839b | Seq. ID No. 3847 | Seq. ID No. 3848 | Cyto | 0 |
| 1842d | Seq. ID No. 3855 | Seq. ID No. 3856 | IM | 10 |
| 1843 | Seq. ID No. 3857 | Seq. ID No. 3858 | Cyto | |
| 1848b | Seq. ID No. 3867 | Seq. ID No. 3868 | IM | 10 |
| 1850a | Seq. ID No. 3869 | Seq. ID No. 3870 | IM | 1 |
| 1850b | Seq. ID No. 3871 | Seq. ID No. 3872 | IM | 1 |
| 1850c | Seq. ID No. 3873 | Seq. ID No. 3874 | IM | 1 |
| 1853a | Seq. ID No. 3875 | Seq. ID No. 3876 | IM | 0 |
| 1853b | Seq. ID No. 3877 | Seq. ID No. 3878 | IM | 0 |
| 1854b | Seq. ID No. 3881 | Seq. ID No. 3882 | IM | 4 |
| 1855 | Seq. ID No. 3883 | Seq. ID No. 3884 | IM | 1 |
| 1856 | Seq. ID No. 3885 | Seq. ID No. 3886 | Cyto | |
| 1857a | Seq. ID No. 3887 | Seq. ID No. 3888 | Cyto | 0 |
| 1857d | Seq. ID No. 3893 | Seq. ID No. 3894 | IM | 8 |
| 1859 | Seq. ID No. 3897 | Seq. ID No. 3898 | Cyto | |
| 1861a | Seq. ID No. 3899 | Seq. ID No. 3900 | Cyto | |
| 1861b | Seq. ID No. 3901 | Seq. ID No. 3902 | Cyto | |
| 1863a | Seq. ID No. 3905 | Seq. ID No. 3906 | Cyto | |
| 1863b | Seq. ID No. 3907 | Seq. ID No. 3908 | Cyto | 0 |
| 1864a | Seq. ID No. 3909 | Seq. ID No. 3910 | Cyto | 0 |
| 1865a | Seq. ID No. 3911 | Seq. ID No. 3912 | IM | 6 |
| 1865b | Seq. ID No. 3913 | Seq. ID No. 3914 | IM | 7 |
| 1869 | Seq. ID No. 3917 | Seq. ID No. 3918 | Cyto | |
| 1870 | Seq. ID No. 3919 | Seq. ID No. 3920 | Cyto | 0 |
| 1873a | Seq. ID No. 3921 | Seq. ID No. 3922 | Cyto | |
| 1875b | Seq. ID No. 3927 | Seq. ID No. 3928 | Cyto | |
| 1875c | Seq. ID No. 3929 | Seq. ID No. 3930 | IM | 1 |
| 1877a | Seq. ID No. 3931 | Seq. ID No. 3932 | Cyto | |
| 1877b | Seq. ID No. 3933 | Seq. ID No. 3934 | Cyto | |
| 1878 | Seq. ID No. 3935 | Seq. ID No. 3936 | IM | |
| 1879 | Seq. ID No. 3937 | Seq. ID No. 3938 | Cyto | 1 |
| 1882 | Seq. ID No. 3939 | Seq. ID No. 3940 | IM | 2 |
| 1884 | Seq. ID No. 3941 | Seq. ID No. 3942 | Cyto | |
| 1886 | Seq. ID No. 3943 | Seq. ID No. 3944 | Cyto | |
| 1887 | Seq. ID No. 3945 | Seq. ID No. 3946 | Cyto | 0 |
| 1888a | Seq. ID No. 3947 | Seq. ID No. 3948 | IM | 10 |
| 1888b | Seq. ID No. 3949 | Seq. ID No. 3950 | IM | 10 |
| 1890 | Seq. ID No. 3951 | Seq. ID No. 3952 | Cyto | |
| 1891a | Seq. ID No. 3953 | Seq. ID No. 3954 | Cyto | |
| 1891b | Seq. ID No. 3955 | Seq. ID No. 3956 | Cyto | |
| 1893 | Seq. ID No. 3957 | Seq. ID No. 3958 | Cyto | 0 |
| 1895 | Seq. ID No. 3961 | Seq. ID No. 3962 | IM | 0 |
| 1896b | Seq. ID No. 3965 | Seq. ID No. 3966 | IM | 2 |
| 1897a | Seq. ID No. 3967 | Seq. ID No. 3968 | Cyto | |
| 1897b | Seq. ID No. 3969 | Seq. ID No. 3970 | Cyto | |
| 1897c | Seq. ID No. 3971 | Seq. ID No. 3972 | Cyto | |
| 1899 | Seq. ID No. 3977 | Seq. ID No. 3978 | Cyto | |
| 1900 | Seq. ID No. 3979 | Seq. ID No. 3980 | Cyto | 1 |
| 1902a | Seq. ID No. 3981 | Seq. ID No. 3982 | Cyto | 0 |
| 1902b | Seq. ID No. 3983 | Seq. ID No. 3984 | Cyto | 0 |
| 1907 | Seq. ID No. 3985 | Seq. ID No. 3986 | Cyto | |
| 1912 | Seq. ID No. 3987 | Seq. ID No. 3988 | Cyto | 0 |
| 1915a | Seq. ID No. 3989 | Seq. ID No. 3990 | Cyto | |
| 1915b | Seq. ID No. 3991 | Seq. ID No. 3992 | Cyto | |
| 1918b | Seq. ID No. 3999 | Seq. ID No. 4000 | IM | 0 |
| 1918c | Seq. ID No. 4001 | Seq. ID No. 4002 | IM | 0 |
| 1918d | Seq. ID No. 4003 | Seq. ID No. 4004 | IM | 0 |
| 1923a | Seq. ID No. 4011 | Seq. ID No. 4012 | Cyto | 0 |
| 1923b | Seq. ID No. 4013 | Seq. ID No. 4014 | Cyto | 0 |
| 1925 | Seq. ID No. 4015 | Seq. ID No. 4016 | Cyto | |
| 1926 | Seq. ID No. 4017 | Seq. ID No. 4018 | Cyto | 1 |
| 1927a | Seq. ID No. 4019 | Seq. ID No. 4020 | Cyto | |
| 1927b | Seq. ID No. 4021 | Seq. ID No. 4022 | Cyto | 0 |
| 1930 | Seq. ID No. 4023 | Seq. ID No. 4024 | Cyto | |
| 1931c | Seq. ID No. 4029 | Seq. ID No. 4030 | IM | 7 |
| 1932 | Seq. ID No. 4031 | Seq. ID No. 4032 | IM | 3 |
| 1933a | Seq. ID No. 4033 | Seq. ID No. 4034 | Cyto | 1 |
| 1933b | Seq. ID No. 4035 | Seq. ID No. 4036 | Cyto | 1 |
| 1934 | Seq. ID No. 4037 | Seq. ID No. 4038 | Cyto | |
| 1939a | Seq. ID No. 4051 | Seq. ID No. 4052 | Cyto | 0 |
| 1939b | Seq. ID No. 4053 | Seq. ID No. 4054 | Cyto | 0 |
| 1939c | Seq. ID No. 4055 | Seq. ID No. 4056 | IM | 0 |
| 1940 | Seq. ID No. 4057 | Seq. ID No. 4058 | Cyto | |
| 1941a | Seq. ID No. 4059 | Seq. ID No. 4060 | IM | 2 |
| 1942 | Seq. ID No. 4063 | Seq. ID No. 4064 | Cyto | |
| 1943a | Seq. ID No. 4065 | Seq. ID No. 4066 | Cyto | 0 |
| 1943b | Seq. ID No. 4067 | Seq. ID No. 4068 | Cyto | 0 |
| 1944 | Seq. ID No. 4069 | Seq. ID No. 4070 | IM | 1 |
| 1946c | Seq. ID No. 4075 | Seq. ID No. 4076 | IM | 10 |
| 1949 | Seq. ID No. 4077 | Seq. ID No. 4078 | Cyto | |
| 1951c | Seq. ID No. 4085 | Seq. ID No. 4086 | IM | 4 |
| 1951d | Seq. ID No. 4087 | Seq. ID No. 4088 | IM | 4 |
| 1953 | Seq. ID No. 4089 | Seq. ID No. 4090 | IM | 1 |
| 1954a | Seq. ID No. 4091 | Seq. ID No. 4092 | IM | 0 |
| 1954b | Seq. ID No. 4093 | Seq. ID No. 4094 | IM | 0 |
| 1954c | Seq. ID No. 4095 | Seq. ID No. 4096 | IM | 0 |
| 1956 | Seq. ID No. 4097 | Seq. ID No. 4098 | Cyto | |
| 1957 | Seq. ID No. 4099 | Seq. ID No. 4100 | Cyto | |
| 1958c | Seq. ID No. 4105 | Seq. ID No. 4106 | IM | 9 |
| 1960a | Seq. ID No. 4107 | Seq. ID No. 4108 | Cyto | 0 |
| 1960b | Seq. ID No. 4109 | Seq. ID No. 4110 | Cyto | 0 |
| 1961 | Seq. ID No. 4113 | Seq. ID No. 4114 | IM | 1 |
| 1962a | Seq. ID No. 4115 | Seq. ID No. 4116 | Cyto | |
| 1962b | Seq. ID No. 4117 | Seq. ID No. 4118 | Cyto | |
| 1965a | Seq. ID No. 4119 | Seq. ID No. 4120 | Cyto | 0 |
| 1965b | Seq. ID No. 4121 | Seq. ID No. 4122 | Cyto | 0 |
| 1966 | Seq. ID No. 4123 | Seq. ID No. 4124 | Cyto | |
| 1967 | Seq. ID No. 4125 | Seq. ID No. 4126 | Cyto | |
| 1968 | Seq. ID No. 4127 | Seq. ID No. 4128 | Cyto | |
| 1969 | Seq. ID No. 4129 | Seq. ID No. 4130 | IM | 3 |
| 1970a | Seq. ID No. 4131 | Seq. ID No. 4132 | Cyto | |
| 1979a | Seq. ID No. 4147 | Seq. ID No. 4148 | Cyto | |
| 1979b | Seq. ID No. 4149 | Seq. ID No. 4150 | Cyto | |
| 1981 | Seq. ID No. 4155 | Seq. ID No. 4156 | Cyto | 1 |
| 1982 | Seq. ID No. 4157 | Seq. ID No. 4158 | IM | |
| 1985a | Seq. ID No. 4159 | Seq. ID No. 4160 | Cyto | |
| 1985b | Seq. ID No. 4161 | Seq. ID No. 4162 | Cyto | |
| 1986 | Seq. ID No. 4163 | Seq. ID No. 4164 | Cyto | |
| 1987a | Seq. ID No. 4165 | Seq. ID No. 4166 | Cyto | |
| 1987b | Seq. ID No. 4167 | Seq. ID No. 4168 | Cyto | |
| 1989a | Seq. ID No. 4169 | Seq. ID No. 4170 | Cyto | |
| 1989b | Seq. ID No. 4171 | Seq. ID No. 4172 | Cyto | |
| 1989c | Seq. ID No. 4173 | Seq. ID No. 4174 | Cyto | |
| 1990a | Seq. ID No. 4175 | Seq. ID No. 4176 | Cyto | |
| 1990b | Seq. ID No. 4177 | Seq. ID No. 4178 | Cyto | |
| 1990c | Seq. ID No. 4179 | Seq. ID No. 4180 | Cyto | |
| 1992a | Seq. ID No. 4183 | Seq. ID No. 4184 | Cyto | |
| 1992b | Seq. ID No. 4185 | Seq. ID No. 4186 | Cyto | |
| 1993a | Seq. ID No. 4187 | Seq. ID No. 4188 | Cyto | |
| 1993b | Seq. ID No. 4189 | Seq. ID No. 4190 | Cyto | |
| 1993c | Seq. ID No. 4191 | Seq. ID No. 4192 | Cyto | |
| 1996 | Seq. ID No. 4193 | Seq. ID No. 4194 | Cyto | 0 |
| 2000 | Seq. ID No. 4195 | Seq. ID No. 4196 | IM | |
| 2001 | Seq. ID No. 4197 | Seq. ID No. 4198 | IM | 0 |
| 2003 | Seq. ID No. 4199 | Seq. ID No. 4200 | Cyto | |
| 2006 | Seq. ID No. 4201 | Seq. ID No. 4202 | Cyto | |
| 2008 | Seq. ID No. 4203 | Seq. ID No. 4204 | IM | 2 |
| 2010 | Seq. ID No. 4205 | Seq. ID No. 4206 | Cyto | |
| 2011a | Seq. ID No. 4207 | Seq. ID No. 4208 | Cyto | 1 |
| 2011b | Seq. ID No. 4209 | Seq. ID No. 4210 | Cyto | 1 |
| 2011c | Seq. ID No. 4211 | Seq. ID No. 4212 | Cyto | 1 |
| 2013a | Seq. ID No. 4213 | Seq. ID No. 4214 | Cyto | |
| 2013b | Seq. ID No. 4215 | Seq. ID No. 4216 | Cyto | |
| 2014c | Seq. ID No. 4217 | Seq. ID No. 4218 | Cyto | |
| 2015 | Seq. ID No. 4219 | Seq. ID No. 4220 | Cyto | |
| 2016e | Seq. ID No. 4229 | Seq. ID No. 4230 | IM | 12 |
| 2016h | Seq. ID No. 4235 | Seq. ID No. 4236 | IM | 13 |
| 2020 | Seq. ID No. 4237 | Seq. ID No. 4238 | IM | 1 |
| 2021 | Seq. ID No. 4239 | Seq. ID No. 4240 | IM | 2 |
| 2023b | Seq. ID No. 4243 | Seq. ID No. 4244 | IM | 7 |
| 2024a | Seq. ID No. 4245 | Seq. ID No. 4246 | Cyto | 1 |
| 2024c | Seq. ID No. 4249 | Seq. ID No. 4250 | IM | 2 |
| 2024d | Seq. ID No. 4251 | Seq. ID No. 4252 | IM | 2 |
| 2027 | Seq. ID No. 4255 | Seq. ID No. 4256 | Cyto | 0 |
| 2033 | Seq. ID No. 4267 | Seq. ID No. 4268 | Cyto | |
| 2036 | Seq. ID No. 4271 | Seq. ID No. 4272 | Cyto | |
| 2037 | Seq. ID No. 4273 | Seq. ID No. 4274 | Cyto | |
| 2040a | Seq. ID No. 4277 | Seq. ID No. 4278 | IM | 3 |
| 2042 | Seq. ID No. 4279 | Seq. ID No. 4280 | IM | 2 |
| 2043b | Seq. ID No. 4281 | Seq. ID No. 4282 | Cyto | |
| 2044 | Seq. ID No. 4283 | Seq. ID No. 4284 | Cyto | 0 |
| 2046a | Seq. ID No. 4289 | Seq. ID No. 4290 | Cyto | |
| 2046b | Seq. ID No. 4291 | Seq. ID No. 4292 | Cyto | |
| 2047a | Seq. ID No. 4293 | Seq. ID No. 4294 | IM | 0 |
| 2047b | Seq. ID No. 4295 | Seq. ID No. 4296 | IM | 0 |
| 2050 | Seq. ID No. 4299 | Seq. ID No. 4300 | Cyto | |
| 2053 | Seq. ID No. 4301 | Seq. ID No. 4302 | Peri | |
| 2054 | Seq. ID No. 4303 | Seq. ID No. 4304 | Cyto | |
| 2055 | Seq. ID No. 4305 | Seq. ID No. 4306 | Cyto | |
| 2057 | Seq. ID No. 4309 | Seq. ID No. 4310 | IM | 0 |
| 2060a | Seq. ID No. 4315 | Seq. ID No. 4316 | Cyto | |
| 2060b | Seq. ID No. 4317 | Seq. ID No. 4318 | Cyto | |
| 2062a | Seq. ID No. 4319 | Seq. ID No. 4320 | Cyto | 0 |
| 2062b | Seq. ID No. 4321 | Seq. ID No. 4322 | Cyto | 0 |
| 2062c | Seq. ID No. 4323 | Seq. ID No. 4324 | Cyto | 0 |
| 2064 | Seq. ID No. 4325 | Seq. ID No. 4326 | Cyto | |
| 2065a | Seq. ID No. 4327 | Seq. ID No. 4328 | Cyto | |
| 2065b | Seq. ID No. 4329 | Seq. ID No. 4330 | Cyto | |
| 2066 | Seq. ID No. 4331 | Seq. ID No. 4332 | IM | |
| 2068 | Seq. ID No. 4333 | Seq. ID No. 4334 | Cyto | |
| 2069a | Seq. ID No. 4335 | Seq. ID No. 4336 | Cyto | |
| 2069b | Seq. ID No. 4337 | Seq. ID No. 4338 | Cyto | |
| 2069c | Seq. ID No. 4339 | Seq. ID No. 4340 | Cyto | |
| 2074a | Seq. ID No. 4341 | Seq. ID No. 4342 | Cyto | |
| 2075a | Seq. ID No. 4345 | Seq. ID No. 4346 | Cyto | |
| 2075b | Seq. ID No. 4347 | Seq. ID No. 4348 | Cyto | |
| 2076a | Seq. ID No. 4349 | Seq. ID No. 4350 | Cyto | |
| 2076b | Seq. ID No. 4351 | Seq. ID No. 4352 | IM | |
| 2076c | Seq. ID No. 4353 | Seq. ID No. 4354 | IM | |
| 2078 | Seq. ID No. 4355 | Seq. ID No. 4356 | IM | 1 |
| 2079a | Seq. ID No. 4357 | Seq. ID No. 4358 | IM | 1 |
| 2079b | Seq. ID No. 4359 | Seq. ID No. 4360 | IM | 1 |
| 2079c | Seq. ID No. 4361 | Seq. ID No. 4362 | IM | 1 |
| 2081a | Seq. ID No. 4363 | Seq. ID No. 4364 | Cyto | |
| 2081b | Seq. ID No. 4365 | Seq. ID No. 4366 | Cyto | |
| 2086a | Seq. ID No. 4367 | Seq. ID No. 4368 | Cyto | |
| 2086b | Seq. ID No. 4369 | Seq. ID No. 4370 | Cyto | |
| 2087a | Seq. ID No. 4371 | Seq. ID No. 4372 | Cyto | |
| 2087b | Seq. ID No. 4373 | Seq. ID No. 4374 | Cyto | |
| 2088 | Seq. ID No. 4375 | Seq. ID No. 4376 | IM | 1 |
| 2091 | Seq. ID No. 4377 | Seq. ID No. 4378 | Cyto | |
| 2093a | Seq. ID No. 4381 | Seq. ID No. 4382 | Cyto | |
| 2094 | Seq. ID No. 4383 | Seq. ID No. 4384 | Cyto | |
| 2096b | Seq. ID No. 4387 | Seq. ID No. 4388 | IM | 9 |
| 2097 | Seq. ID No. 4393 | Seq. ID No. 4394 | Cyto | 0 |
| 2098b | Seq. ID No. 4395 | Seq. ID No. 4396 | IM | 1 |
| 2100 | Seq. ID No. 4397 | Seq. ID No. 4398 | Cyto | 1 |
| 2104 | Seq. ID No. 4401 | Seq. ID No. 4402 | IM | 1 |
| 2107a | Seq. ID No. 4405 | Seq. ID No. 4406 | Cyto | 0 |
| 2107b | Seq. ID No. 4407 | Seq. ID No. 4408 | Cyto | 0 |
| 2109a | Seq. ID No. 4409 | Seq. ID No. 4410 | Cyto | |
| 2109b | Seq. ID No. 4411 | Seq. ID No. 4412 | Cyto | |
| 2110a | Seq. ID No. 4413 | Seq. ID No. 4414 | Cyto | 1 |
| 2110b | Seq. ID No. 4415 | Seq. ID No. 4416 | Cyto | 1 |
| 2110c | Seq. ID No. 4417 | Seq. ID No. 4418 | Cyto | 1 |
| 2112a | Seq. ID No. 4419 | Seq. ID No. 4420 | Cyto | 1 |
| 2112b | Seq. ID No. 4421 | Seq. ID No. 4422 | Cyto | 1 |
| 2112c | Seq. ID No. 4423 | Seq. ID No. 4424 | IM | 1 |
| 2114b | Seq. ID No. 4425 | Seq. ID No. 4426 | IM | 1 |
| 2115a | Seq. ID No. 4427 | Seq. ID No. 4428 | Cyto | |
| 2115b | Seq. ID No. 4429 | Seq. ID No. 4430 | Cyto | |
| 2115c | Seq. ID No. 4431 | Seq. ID No. 4432 | IM | |
| 2116 | Seq. ID No. 4433 | Seq. ID No. 4434 | Cyto | |
| 2117 | Seq. ID No. 4435 | Seq. ID No. 4436 | Cyto | 0 |
| 2123 | Seq. ID No. 4443 | Seq. ID No. 4444 | Cyto | |
| 2124a | Seq. ID No. 4445 | Seq. ID No. 4446 | Cyto | |
| 2124b | Seq. ID No. 4447 | Seq. ID No. 4448 | Cyto | |
| 2125a | Seq. ID No. 4449 | Seq. ID No. 4450 | Cyto | |
| 2125b | Seq. ID No. 4451 | Seq. ID No. 4452 | Cyto | |
| 2127a | Seq. ID No. 4453 | Seq. ID No. 4454 | IM | 1 |
| 2127b | Seq. ID No. 4455 | Seq. ID No. 4456 | IM | 1 |
| 2128a | Seq. ID No. 4457 | Seq. ID No. 4458 | Cyto | |
| 2128b | Seq. ID No. 4459 | Seq. ID No. 4460 | Cyto | |
| 2133 | Seq. ID No. 4461 | Seq. ID No. 4462 | Cyto | 0 |
| 2138a | Seq. ID No. 4463 | Seq. ID No. 4464 | Cyto | 0 |
| 2138b | Seq. ID No. 4465 | Seq. ID No. 4466 | Cyto | 0 |
| 2138c | Seq. ID No. 4467 | Seq. ID No. 4468 | Cyto | 0 |
| 2140b | Seq. ID No. 4471 | Seq. ID No. 4472 | IM | 5 |
| 2142b | Seq. ID No. 4475 | Seq. ID No. 4476 | IM | 7 |
| 2144a | Seq. ID No. 4477 | Seq. ID No. 4478 | Cyto | |
| 2144b | Seq. ID No. 4479 | Seq. ID No. 4480 | Cyto | |
| 2147c | Seq. ID No. 4487 | Seq. ID No. 4488 | IM | 5 |
| 2149a | Seq. ID No. 4489 | Seq. ID No. 4490 | Cyto | |
| 2149b | Seq. ID No. 4491 | Seq. ID No. 4492 | Cyto | |
| 2152a | Seq. ID No. 4499 | Seq. ID No. 4500 | Cyto | |
| 2153 | Seq. ID No. 4501 | Seq. ID No. 4502 | Cyto | |
| 2155a | Seq. ID No. 4503 | Seq. ID No. 4504 | Cyto | 0 |
| 2155b | Seq. ID No. 4505 | Seq. ID No. 4506 | Cyto | 0 |
| 2156a | Seq. ID No. 4507 | Seq. ID No. 4508 | Cyto | |
| 2156b | Seq. ID No. 4509 | Seq. ID No. 4510 | Cyto | |
| 2156c | Seq. ID No. 4511 | Seq. ID No. 4512 | Cyto | |
| 2160a | Seq. ID No. 4517 | Seq. ID No. 4518 | Cyto | |
| 2160b | Seq. ID No. 4519 | Seq. ID No. 4520 | Cyto | |
| 2160c | Seq. ID No. 4521 | Seq. ID No. 4522 | Cyto | |
| 2160d | Seq. ID No. 4523 | Seq. ID No. 4524 | Cyto | |
| 2161a | Seq. ID No. 4525 | Seq. ID No. 4526 | Cyto | 1 |
| 2164 | Seq. ID No. 4531 | Seq. ID No. 4532 | IM | 0 |
| 2166a | Seq. ID No. 4535 | Seq. ID No. 4536 | Cyto | 1 |
| 2166b | Seq. ID No. 4537 | Seq. ID No. 4538 | Cyto | 2 |
| 2169 | Seq. ID No. 4539 | Seq. ID No. 4540 | Cyto | |
| 2171 | Seq. ID No. 4541 | Seq. ID No. 4542 | Cyto | 0 |
| 2172 | Seq. ID No. 4543 | Seq. ID No. 4544 | Cyto | |
| 2173a | Seq. ID No. 4545 | Seq. ID No. 4546 | Cyto | 0 |
| 2173b | Seq. ID No. 4547 | Seq. ID No. 4548 | Cyto | 0 |
| 2174 | Seq. ID No. 4549 | Seq. ID No. 4550 | IM | 0 |
| 2175 | Seq. ID No. 4551 | Seq. ID No. 4552 | IM | 2 |
| 2176a | Seq. ID No. 4553 | Seq. ID No. 4554 | Cyto | 0 |
| 2176b | Seq. ID No. 4555 | Seq. ID No. 4556 | IM | 1 |
| 2176c | Seq. ID No. 4557 | Seq. ID No. 4558 | Cyto | 1 |
| 2176d | Seq. ID No. 4559 | Seq. ID No. 4560 | Cyto | 1 |
| 2177b | Seq. ID No. 4561 | Seq. ID No. 4562 | IM | 1 |
| 2179a | Seq. ID No. 4563 | Seq. ID No. 4564 | Cyto | 0 |
| 2179b | Seq. ID No. 4565 | Seq. ID No. 4566 | Cyto | 0 |
| 2180 | Seq. ID No. 4567 | Seq. ID No. 4568 | Cyto | 0 |
| 2181a | Seq. ID No. 4569 | Seq. ID No. 4570 | Cyto | 0 |
| 2181b | Seq. ID No. 4571 | Seq. ID No. 4572 | Cyto | 0 |
| 2182 | Seq. ID No. 4573 | Seq. ID No. 4574 | IM | 1 |
| 2183 | Seq. ID No. 4575 | Seq. ID No. 4576 | Cyto | |
| 2185 | Seq. ID No. 4577 | Seq. ID No. 4578 | Cyto | |
| 2186a | Seq. ID No. 4579 | Seq. ID No. 4580 | Cyto | |
| 2186b | Seq. ID No. 4581 | Seq. ID No. 4582 | Cyto | |
| 2189a | Seq. ID No. 4583 | Seq. ID No. 4584 | Cyto | |
| 2189b | Seq. ID No. 4585 | Seq. ID No. 4586 | Cyto | |
| 2193 | Seq. ID No. 4591 | Seq. ID No. 4592 | Cyto | |
| 2194a | Seq. ID No. 4593 | Seq. ID No. 4594 | Cyto | |
| 2194c | Seq. ID No. 4595 | Seq. ID No. 4596 | Cyto | |
| 2196 | Seq. ID No. 4597 | Seq. ID No. 4598 | Cyto | |
| 2197a | Seq. ID No. 4599 | Seq. ID No. 4600 | Cyto | |
| 2197b | Seq. ID No. 4601 | Seq. ID No. 4602 | Cyto | |
| 2197c | Seq. ID No. 4603 | Seq. ID No. 4604 | Cyto | |
| 2201a | Seq. ID No. 4605 | Seq. ID No. 4606 | Cyto | 0 |
| 2201b | Seq. ID No. 4607 | Seq. ID No. 4608 | Cyto | 0 |
| 2203 | Seq. ID No. 4609 | Seq. ID No. 4610 | Cyto | |
| 2204 | Seq. ID No. 4611 | Seq. ID No. 4612 | Cyto | 0 |
| 2206a | Seq. ID No. 4613 | Seq. ID No. 4614 | Cyto | |
| 2206b | Seq. ID No. 4615 | Seq. ID No. 4616 | Cyto | |
| 2208a | Seq. ID No. 4617 | Seq. ID No. 4618 | Cyto | 0 |
| 2208b | Seq. ID No. 4619 | Seq. ID No. 4620 | Cyto | 0 |
| 2208c | Seq. ID No. 4621 | Seq. ID No. 4622 | Cyto | 0 |
| 2209 | Seq. ID No. 4623 | Seq. ID No. 4624 | Cyto | |
| 2212a | Seq. ID No. 4625 | Seq. ID No. 4626 | IM | 1 |
| 2212b | Seq. ID No. 4627 | Seq. ID No. 4628 | IM | 1 |
| 2215a | Seq. ID No. 4629 | Seq. ID No. 4630 | Cyto | 0 |
| 2215b | Seq. ID No. 4631 | Seq. ID No. 4632 | Cyto | 0 |
| 2216a | Seq. ID No. 4633 | Seq. ID No. 4634 | Cyto | |
| 2216b | Seq. ID No. 4635 | Seq. ID No. 4636 | Cyto | |
| 2218 | Seq. ID No. 4637 | Seq. ID No. 4638 | Cyto | 1 |
| 2220a | Seq. ID No. 4645 | Seq. ID No. 4646 | Cyto | |
| 2220b | Seq. ID No. 4647 | Seq. ID No. 4648 | Cyto | |
| 2220c | Seq. ID No. 4649 | Seq. ID No. 4650 | Cyto | |
| 2221 | Seq. ID No. 4651 | Seq. ID No. 4652 | Cyto | |
| 2222 | Seq. ID No. 4653 | Seq. ID No. 4654 | Cyto | 0 |
| 2223a | Seq. ID No. 4655 | Seq. ID No. 4656 | Cyto | |
| 2223b | Seq. ID No. 4657 | Seq. ID No. 4658 | Cyto | |
| 2226 | Seq. ID No. 4661 | Seq. ID No. 4662 | Cyto | 0 |
| 2227 | Seq. ID No. 4663 | Seq. ID No. 4664 | Cyto | |
| 2228a | Seq. ID No. 4665 | Seq. ID No. 4666 | Cyto | |
| 2228b | Seq. ID No. 4667 | Seq. ID No. 4668 | Cyto | |
| 2228c | Seq. ID No. 4669 | Seq. ID No. 4670 | Cyto | |
| 2231 | Seq. ID No. 4671 | Seq. ID No. 4672 | Cyto | |
| 2232 | Seq. ID No. 4673 | Seq. ID No. 4674 | Cyto | |
| 2234a | Seq. ID No. 4675 | Seq. ID No. 4676 | IM | 1 |
| 2234b | Seq. ID No. 4677 | Seq. ID No. 4678 | IM | 1 |
| 2234c | Seq. ID No. 4679 | Seq. ID No. 4680 | IM | 1 |
| 2236b | Seq. ID No. 4683 | Seq. ID No. 4684 | IM | 4 |
| 2238 | Seq. ID No. 4685 | Seq. ID No. 4686 | Cyto | |
| 2240a | Seq. ID No. 4689 | Seq. ID No. 4690 | Cyto | 0 |
| 2240b | Seq. ID No. 4691 | Seq. ID No. 4692 | Cyto | 1 |
| 2240c | Seq. ID No. 4693 | Seq. ID No. 4694 | Cyto | 1 |
| 2244 | Seq. ID No. 4697 | Seq. ID No. 4698 | Cyto | |
| 2245 | Seq. ID No. 4699 | Seq. ID No. 4700 | OM | 1 |
| 2246 | Seq. ID No. 4701 | Seq. ID No. 4702 | IM | |
| 2248 | Seq. ID No. 4703 | Seq. ID No. 4704 | Cyto | |
| 2253d | Seq. ID No. 4713 | Seq. ID No. 4714 | IM | 7 |
| 2256 | Seq. ID No. 4719 | Seq. ID No. 4720 | Cyto | |
| 2257 | Seq. ID No. 4721 | Seq. ID No. 4722 | IM | 0 |
| 2259a | Seq. ID No. 4723 | Seq. ID No. 4724 | Cyto | |
| 2259b | Seq. ID No. 4725 | Seq. ID No. 4726 | Cyto | |
| 2260b | Seq. ID No. 4727 | Seq. ID No. 4728 | IM | |
| 2260c | Seq. ID No. 4729 | Seq. ID No. 4730 | IM | |
| 2262 | Seq. ID No. 4731 | Seq. ID No. 4732 | Cyto | |
| 2263 | Seq. ID No. 4733 | Seq. ID No. 4734 | Cyto | 1 |
| 2264a | Seq. ID No. 4735 | Seq. ID No. 4736 | Peri | 1 |
| 2267 | Seq. ID No. 4739 | Seq. ID No. 4740 | Cyto | 0 |
| 2268 | Seq. ID No. 4741 | Seq. ID No. 4742 | Cyto | 0 |
| 2269 | Seq. ID No. 4743 | Seq. ID No. 4744 | Cyto | |
| 2270 | Seq. ID No. 4745 | Seq. ID No. 4746 | IM | 1 |
| 2272 | Seq. ID No. 4747 | Seq. ID No. 4748 | Cyto | 1 |
| 2273 | Seq. ID No. 4749 | Seq. ID No. 4750 | Cyto | |
| 2274 | Seq. ID No. 4751 | Seq. ID No. 4752 | Cyto | |
| 2275a | Seq. ID No. 4753 | Seq. ID No. 4754 | IM | 1 |
| 2275b | Seq. ID No. 4755 | Seq. ID No. 4756 | IM | 1 |
| 2275c | Seq. ID No. 4757 | Seq. ID No. 4758 | IM | 1 |
| 2276b | Seq. ID No. 4761 | Seq. ID No. 4762 | Cyto | 1 |
| 2276c | Seq. ID No. 4763 | Seq. ID No. 4764 | Cyto | 1 |
| 2276d | Seq. ID No. 4765 | Seq. ID No. 4766 | Cyto | 1 |
| 2277a | Seq. ID No. 4767 | Seq. ID No. 4768 | Cyto | 1 |
| 2277b | Seq. ID No. 4769 | Seq. ID No. 4770 | Cyto | 1 |
| 2278a | Seq. ID No. 4771 | Seq. ID No. 4772 | Cyto | |
| 2278b | Seq. ID No. 4773 | Seq. ID No. 4774 | Cyto | |
| 2278c | Seq. ID No. 4775 | Seq. ID No. 4776 | Cyto | 0 |
| 2278d | Seq. ID No. 4777 | Seq. ID No. 4778 | Cyto | 0 |
| 2280 | Seq. ID No. 4779 | Seq. ID No. 4780 | Cyto | |
| 2284 | Seq. ID No. 4781 | Seq. ID No. 4782 | Cyto | 0 |
| 2285 | Seq. ID No. 4783 | Seq. ID No. 4784 | Cyto | |
| 2286a | Seq. ID No. 4785 | Seq. ID No. 4786 | Cyto | |
| 2286b | Seq. ID No. 4787 | Seq. ID No. 4788 | Cyto | |
| 2288 | Seq. ID No. 4789 | Seq. ID No. 4790 | Peri | 1 |
| 2290 | Seq. ID No. 4791 | Seq. ID No. 4792 | Cyto | |
| 2291a | Seq. ID No. 4793 | Seq. ID No. 4794 | Cyto | |
| 2291b | Seq. ID No. 4795 | Seq. ID No. 4796 | Cyto | |
| 2291c | Seq. ID No. 4797 | Seq. ID No. 4798 | Cyto | |
| 2292a | Seq. ID No. 4799 | Seq. ID No. 4800 | Cyto | 0 |
| 2292b | Seq. ID No. 4801 | Seq. ID No. 4802 | Cyto | 0 |
| 2292c | Seq. ID No. 4803 | Seq. ID No. 4804 | Cyto | 0 |
| 2294 | Seq. ID No. 4805 | Seq. ID No. 4806 | Cyto | |
| 2297 | Seq. ID No. 4807 | Seq. ID No. 4808 | Cyto | |
| 2298 | Seq. ID No. 4809 | Seq. ID No. 4810 | Cyto | |
| 2299 | Seq. ID No. 4811 | Seq. ID No. 4812 | Cyto | |
| 2301 | Seq. ID No. 4815 | Seq. ID No. 4816 | IM | 7 |
| 2302 | Seq. ID No. 4817 | Seq. ID No. 4818 | IM | 1 |
| 2303 | Seq. ID No. 4819 | Seq. ID No. 4820 | IM | 1 |
| 2304 | Seq. ID No. 4821 | Seq. ID No. 4822 | Cyto | |
| 2306c | Seq. ID No. 4823 | Seq. ID No. 4824 | Cyto | |
| 2307 | Seq. ID No. 4825 | Seq. ID No. 4826 | IM | 4 |
| 2309a | Seq. ID No. 4829 | Seq. ID No. 4830 | IM | 2 |
| 2309b | Seq. ID No. 4831 | Seq. ID No. 4832 | IM | 2 |
| 2309c | Seq. ID No. 4833 | Seq. ID No. 4834 | IM | 2 |
| 2310 | Seq. ID No. 4835 | Seq. ID No. 4836 | IM | 2 |
| 2311 | Seq. ID No. 4837 | Seq. ID No. 4838 | Cyto | |
| 2312a | Seq. ID No. 4839 | Seq. ID No. 4840 | Cyto | |
| 2312b | Seq. ID No. 4841 | Seq. ID No. 4842 | Cyto | |
| 2312c | Seq. ID No. 4843 | Seq. ID No. 4844 | Cyto | |
| 2314 | Seq. ID No. 4845 | Seq. ID No. 4846 | Cyto | 0 |
| 2315 | Seq. ID No. 4847 | Seq. ID No. 4848 | Cyto | |
| 2316 | Seq. ID No. 4849 | Seq. ID No. 4850 | Cyto | 1 |
| 2317 | Seq. ID No. 4851 | Seq. ID No. 4852 | IM | 2 |
| 2321a | Seq. ID No. 4855 | Seq. ID No. 4856 | Cyto | |
| 2321b | Seq. ID No. 4857 | Seq. ID No. 4858 | Cyto | |
| 2321c | Seq. ID No. 4859 | Seq. ID No. 4860 | Cyto | |
| 2322 | Seq. ID No. 4861 | Seq. ID No. 4862 | IM | 0 |
| 2327 | Seq. ID No. 4865 | Seq. ID No. 4866 | Cyto | |
| 2328 | Seq. ID No. 4867 | Seq. ID No. 4868 | Cyto | |
| 2330 | Seq. ID No. 4877 | Seq. ID No. 4878 | Cyto | |
| 2331 | Seq. ID No. 4879 | Seq. ID No. 4880 | IM | 1 |
| 2333 | Seq. ID No. 4881 | Seq. ID No. 4882 | Cyto | |
| 2335 | Seq. ID No. 4883 | Seq. ID No. 4884 | Cyto | |
| 2336 | Seq. ID No. 4885 | Seq. ID No. 4886 | Cyto | |
| 2337b | Seq. ID No. 4887 | Seq. ID No. 4888 | Cyto | |
| 2338a | Seq. ID No. 4889 | Seq. ID No. 4890 | Cyto | |
| 2338b | Seq. ID No. 4891 | Seq. ID No. 4892 | Cyto | |
| 2338c | Seq. ID No. 4893 | Seq. ID No. 4894 | Cyto | |
| 2342a | Seq. ID No. 4895 | Seq. ID No. 4896 | Cyto | |
| 2342b | Seq. ID No. 4897 | Seq. ID No. 4898 | Cyto | |
| 2342c | Seq. ID No. 4899 | Seq. ID No. 4900 | Cyto | |
| 2344 | Seq. ID No. 4901 | Seq. ID No. 4902 | IM | 0 |
| 2345 | Seq. ID No. 4903 | Seq. ID No. 4904 | Cyto | 0 |
| 2347 | Seq. ID No. 4905 | Seq. ID No. 4906 | Cyto | 1 |
| 2348 | Seq. ID No. 4907 | Seq. ID No. 4908 | Cyto | 0 |
| 2349a | Seq. ID No. 4909 | Seq. ID No. 4910 | Cyto | |
| 2349b | Seq. ID No. 4911 | Seq. ID No. 4912 | Cyto | |
| 2350a | Seq. ID No. 4913 | Seq. ID No. 4914 | Cyto | |
| 2353 | Seq. ID No. 4915 | Seq. ID No. 4916 | Cyto | |
| 2354 | Seq. ID No. 4917 | Seq. ID No. 4918 | Cyto | |
| 2361a | Seq. ID No. 4925 | Seq. ID No. 4926 | Cyto | |
| 2361b | Seq. ID No. 4927 | Seq. ID No. 4928 | Cyto | |
| 2365a | Seq. ID No. 4933 | Seq. ID No. 4934 | Cyto | 0 |
| 2365b | Seq. ID No. 4935 | Seq. ID No. 4936 | Cyto | 0 |
| 2366a | Seq. ID No. 4937 | Seq. ID No. 4938 | Cyto | |
| 2366b | Seq. ID No. 4939 | Seq. ID No. 4940 | Cyto | |
| 2367 | Seq. ID No. 4941 | Seq. ID No. 4942 | Cyto | |
| 2368 | Seq. ID No. 4943 | Seq. ID No. 4944 | Cyto | |
| 2369 | Seq. ID No. 4945 | Seq. ID No. 4946 | Cyto | |
| 2370 | Seq. ID No. 4947 | Seq. ID No. 4948 | IM | 1 |
| 2373 | Seq. ID No. 4953 | Seq. ID No. 4954 | Cyto | |
| 2374a | Seq. ID No. 4955 | Seq. ID No. 4956 | Cyto | 0 |
| 2374b | Seq. ID No. 4957 | Seq. ID No. 4958 | Cyto | 0 |
| 2376 | Seq. ID No. 4959 | Seq. ID No. 4960 | IM | |
| 2377a | Seq. ID No. 4961 | Seq. ID No. 4962 | Cyto | |
| 2377b | Seq. ID No. 4963 | Seq. ID No. 4964 | IM | 1 |
| 2378 | Seq. ID No. 4965 | Seq. ID No. 4966 | Cyto | |
| 2379a | Seq. ID No. 4967 | Seq. ID No. 4968 | Cyto | |
| 2379b | Seq. ID No. 4969 | Seq. ID No. 4970 | Cyto | |
| 2380 | Seq. ID No. 4971 | Seq. ID No. 4972 | Cyto | |
| 2381 | Seq. ID No. 4973 | Seq. ID No. 4974 | Cyto | |
| 2384a | Seq. ID No. 4977 | Seq. ID No. 4978 | Cyto | 0 |
| 2384b | Seq. ID No. 4979 | Seq. ID No. 4980 | Cyto | 0 |
| 2386b | Seq. ID No. 4981 | Seq. ID No. 4982 | IM | 0 |
| 2386c | Seq. ID No. 4983 | Seq. ID No. 4984 | IM | 0 |
| 2387 | Seq. ID No. 4985 | Seq. ID No. 4986 | IM | 5 |
| 2391 | Seq. ID No. 4989 | Seq. ID No. 4990 | IM | 0 |
| 2392 | Seq. ID No. 4991 | Seq. ID No. 4992 | Cyto | 1 |
| 2394 | Seq. ID No. 4993 | Seq. ID No. 4994 | IM | 1 |
| 2395 | Seq. ID No. 4995 | Seq. ID No. 4996 | IM | |
| 2396 | Seq. ID No. 4997 | Seq. ID No. 4998 | Cyto | 0 |
| 2398 | Seq. ID No. 5001 | Seq. ID No. 5002 | Cyto | |
| 2399a | Seq. ID No. 5003 | Seq. ID No. 5004 | Cyto | |
| 2399b | Seq. ID No. 5005 | Seq. ID No. 5006 | Cyto | |
| 2399c | Seq. ID No. 5007 | Seq. ID No. 5008 | Cyto | |
| 2401 | Seq. ID No. 5009 | Seq. ID No. 5010 | Cyto | |
| 2402a | Seq. ID No. 5011 | Seq. ID No. 5012 | IM | 1 |
| 2403a | Seq. ID No. 5015 | Seq. ID No. 5016 | Cyto | 0 |
| 2403b | Seq. ID No. 5017 | Seq. ID No. 5018 | Cyto | 0 |
| 2404a | Seq. ID No. 5019 | Seq. ID No. 5020 | Cyto | 0 |
| 2404b | Seq. ID No. 5021 | Seq. ID No. 5022 | Cyto | 0 |
| 2407a | Seq. ID No. 5023 | Seq. ID No. 5024 | IM | 0 |
| 2409a | Seq. ID No. 5025 | Seq. ID No. 5026 | Cyto | |
| 2409b | Seq. ID No. 5027 | Seq. ID No. 5028 | Cyto | |
| 2410 | Seq. ID No. 5029 | Seq. ID No. 5030 | Cyto | 0 |
| 2413a | Seq. ID No. 5033 | Seq. ID No. 5034 | Cyto | 0 |
| 2413b | Seq. ID No. 5035 | Seq. ID No. 5036 | Peri | 0 |
| 2415a | Seq. ID No. 5037 | Seq. ID No. 5038 | Cyto | |
| 2415b | Seq. ID No. 5039 | Seq. ID No. 5040 | IM | |
| 2416b | Seq. ID No. 5041 | Seq. ID No. 5042 | OM | 1 |
| 2416c | Seq. ID No. 5043 | Seq. ID No. 5044 | IM | 1 |
| 2417a | Seq. ID No. 5045 | Seq. ID No. 5046 | Cyto | 1 |
| 2417b | Seq. ID No. 5047 | Seq. ID No. 5048 | Cyto | 1 |
| 2418a | Seq. ID No. 5049 | Seq. ID No. 5050 | Cyto | |
| 2418b | Seq. ID No. 5051 | Seq. ID No. 5052 | Cyto | |
| 2420 | Seq. ID No. 5053 | Seq. ID No. 5054 | Cyto | 0 |
| 2421 | Seq. ID No. 5055 | Seq. ID No. 5056 | Cyto | |
| 2422b | Seq. ID No. 5057 | Seq. ID No. 5058 | Cyto | |
| 2422c | Seq. ID No. 5059 | Seq. ID No. 5060 | Cyto | |
| 2424a | Seq. ID No. 5065 | Seq. ID No. 5066 | Cyto | 1 |
| 2424b | Seq. ID No. 5067 | Seq. ID No. 5068 | Cyto | 1 |
| 2426 | Seq. ID No. 5071 | Seq. ID No. 5072 | Cyto | |
| 2428a | Seq. ID No. 5073 | Seq. ID No. 5074 | IM | 3 |
| 2428b | Seq. ID No. 5075 | Seq. ID No. 5076 | IM | 3 |
| 2428c | Seq. ID No. 5077 | Seq. ID No. 5078 | IM | 3 |
| 2429 | Seq. ID No. 5079 | Seq. ID No. 5080 | Cyto | |
| 2432 | Seq. ID No. 5081 | Seq. ID No. 5082 | Cyto | 0 |
| 2433 | Seq. ID No. 5083 | Seq. ID No. 5084 | IM | 1 |
| 2440 | Seq. ID No. 5087 | Seq. ID No. 5088 | Cyto | 0 |
| 2442a | Seq. ID No. 5089 | Seq. ID No. 5090 | Cyto | |
| 2442b | Seq. ID No. 5091 | Seq. ID No. 5092 | Cyto | |
| 2443 | Seq. ID No. 5093 | Seq. ID No. 5094 | Cyto | |
| 2444a | Seq. ID No. 5095 | Seq. ID No. 5096 | Cyto | |
| 2444b | Seq. ID No. 5097 | Seq. ID No. 5098 | Cyto | |
| 2444c | Seq. ID No. 5099 | Seq. ID No. 5100 | Cyto | |
| 2444d | Seq. ID No. 5101 | Seq. ID No. 5102 | Cyto | |
| 2448b | Seq. ID No. 5105 | Seq. ID No. 5106 | Cyto | |
| 2449a | Seq. ID No. 5107 | Seq. ID No. 5108 | Cyto | |
| 2449b | Seq. ID No. 5109 | Seq. ID No. 5110 | Cyto | |
| 2451 | Seq. ID No. 5111 | Seq. ID No. 5112 | Cyto | |
| 2452 | Seq. ID No. 5113 | Seq. ID No. 5114 | IM | 0 |
| 2455 | Seq. ID No. 5117 | Seq. ID No. 5118 | Cyto | |
| 2456a | Seq. ID No. 5119 | Seq. ID No. 5120 | IM | 4 |
| 2456b | Seq. ID No. 5121 | Seq. ID No. 5122 | IM | 5 |
| 2456e | Seq. ID No. 5127 | Seq. ID No. 5128 | IM | 9 |
| 2461a | Seq. ID No. 5143 | Seq. ID No. 5144 | Cyto | |
| 2461b | Seq. ID No. 5145 | Seq. ID No. 5146 | Cyto | |
| 2462a | Seq. ID No. 5147 | Seq. ID No. 5148 | Cyto | 0 |
| 2462b | Seq. ID No. 5149 | Seq. ID No. 5150 | Cyto | 0 |
| 2463a | Seq. ID No. 5151 | Seq. ID No. 5152 | IM | 1 |
| 2464 | Seq. ID No. 5153 | Seq. ID No. 5154 | IM | 1 |
| 2465a | Seq. ID No. 5155 | Seq. ID No. 5156 | Cyto | |
| 2465b | Seq. ID No. 5157 | Seq. ID No. 5158 | Cyto | |
| 2468b | Seq. ID No. 5161 | Seq. ID No. 5162 | IM | 1 |
| 2469a | Seq. ID No. 5163 | Seq. ID No. 5164 | Cyto | |
| 2470 | Seq. ID No. 5167 | Seq. ID No. 5168 | Cyto | 0 |
| 2471 | Seq. ID No. 5169 | Seq. ID No. 5170 | Cyto | |
| 2473 | Seq. ID No. 5171 | Seq. ID No. 5172 | Cyto | |
| 2474a | Seq. ID No. 5173 | Seq. ID No. 5174 | IM | 3 |
| 2474c | Seq. ID No. 5177 | Seq. ID No. 5178 | IM | 3 |
| 2475a | Seq. ID No. 5179 | Seq. ID No. 5180 | IM | 1 |
| 2475b | Seq. ID No. 5181 | Seq. ID No. 5182 | IM | 1 |
| 2475c | Seq. ID No. 5183 | Seq. ID No. 5184 | IM | 1 |
| 2477 | Seq. ID No. 5185 | Seq. ID No. 5186 | Cyto | |
| 2480 | Seq. ID No. 5187 | Seq. ID No. 5188 | Cyto | |
| 2481a | Seq. ID No. 5189 | Seq. ID No. 5190 | IM | 0 |
| 2481b | Seq. ID No. 5191 | Seq. ID No. 5192 | IM | 1 |
| 2482 | Seq. ID No. 5193 | Seq. ID No. 5194 | IM | 2 |
| 2483 | Seq. ID No. 5195 | Seq. ID No. 5196 | Cyto | |
| 2485 | Seq. ID No. 5201 | Seq. ID No. 5202 | Cyto | 1 |
| 2489 | Seq. ID No. 5203 | Seq. ID No. 5204 | Cyto | 1 |
| 2492b | Seq. ID No. 5209 | Seq. ID No. 5210 | IM | 1 |
| 2494 | Seq. ID No. 5211 | Seq. ID No. 5212 | IM | 0 |
| 2495b | Seq. ID No. 5215 | Seq. ID No. 5216 | IM | 10 |
| 2497a | Seq. ID No. 5217 | Seq. ID No. 5218 | IM | 1 |
| 2498b | Seq. ID No. 5221 | Seq. ID No. 5222 | IM | 1 |
| 2500b | Seq. ID No. 5225 | Seq. ID No. 5226 | IM | 1 |
| 2500c | Seq. ID No. 5227 | Seq. ID No. 5228 | IM | 1 |
| 2501a | Seq. ID No. 5229 | Seq. ID No. 5230 | Cyto | |
| 2501b | Seq. ID No. 5231 | Seq. ID No. 5232 | Cyto | |
| 2504a | Seq. ID No. 5233 | Seq. ID No. 5234 | Cyto | |
| 2504b | Seq. ID No. 5235 | Seq. ID No. 5236 | Cyto | |
| 2505 | Seq. ID No. 5237 | Seq. ID No. 5238 | IM | 3 |
| 2506 | Seq. ID No. 5239 | Seq. ID No. 5240 | Cyto | |
| 2507b | Seq. ID No. 5241 | Seq. ID No. 5242 | Cyto | |
| 2507c | Seq. ID No. 5243 | Seq. ID No. 5244 | Cyto | |
| 2510 | Seq. ID No. 5245 | Seq. ID No. 5246 | Cyto | |
| 2512c | Seq. ID No. 5247 | Seq. ID No. 5248 | IM | |
| 2513a | Seq. ID No. 5249 | Seq. ID No. 5250 | Cyto | |
| 2513b | Seq. ID No. 5251 | Seq. ID No. 5252 | Cyto | |
| 2513c | Seq. ID No. 5253 | Seq. ID No. 5254 | Cyto | |
| 2514 | Seq. ID No. 5255 | Seq. ID No. 5256 | Cyto | |
| 2515 | Seq. ID No. 5257 | Seq. ID No. 5258 | Cyto | |
| 2516a | Seq. ID No. 5259 | Seq. ID No. 5260 | Cyto | |
| 2516b | Seq. ID No. 5261 | Seq. ID No. 5262 | Cyto | |
| 2517a | Seq. ID No. 5263 | Seq. ID No. 5264 | Cyto | |
| 2517b | Seq. ID No. 5265 | Seq. ID No. 5266 | Cyto | |
| 2519a | Seq. ID No. 5269 | Seq. ID No. 5270 | Cyto | |
| 2519b | Seq. ID No. 5271 | Seq. ID No. 5272 | Cyto | 0 |
| 2523 | Seq. ID No. 5283 | Seq. ID No. 5284 | Cyto | 0 |
| 2525b | Seq. ID No. 5287 | Seq. ID No. 5288 | IM | 1 |
| 2527a | Seq. ID No. 5289 | Seq. ID No. 5290 | Cyto | |
| 2527b | Seq. ID No. 5291 | Seq. ID No. 5292 | Cyto | |
| 2528a | Seq. ID No. 5293 | Seq. ID No. 5294 | Cyto | 0 |
| 2528b | Seq. ID No. 5295 | Seq. ID No. 5296 | Cyto | 0 |
| 2528c | Seq. ID No. 5297 | Seq. ID No. 5298 | Cyto | 0 |
| 2528d | Seq. ID No. 5299 | Seq. ID No. 5300 | Cyto | 0 |
| 2532b | Seq. ID No. 5301 | Seq. ID No. 5302 | Cyto | 1 |
| 2532c | Seq. ID No. 5303 | Seq. ID No. 5304 | Cyto | 1 |
| 2534 | Seq. ID No. 5305 | Seq. ID No. 5306 | Cyto | |
| 2535a | Seq. ID No. 5307 | Seq. ID No. 5308 | Cyto | |
| 2535b | Seq. ID No. 5309 | Seq. ID No. 5310 | Cyto | |
| 2538 | Seq. ID No. 5311 | Seq. ID No. 5312 | Cyto | |
| 2541 | Seq. ID No. 5313 | Seq. ID No. 5314 | Cyto | |
| 2542a | Seq. ID No. 5315 | Seq. ID No. 5316 | Cyto | |
| 2542b | Seq. ID No. 5317 | Seq. ID No. 5318 | Cyto | 0 |
| 2542c | Seq. ID No. 5319 | Seq. ID No. 5320 | Cyto | 0 |
| 2543 | Seq. ID No. 5321 | Seq. ID No. 5322 | Cyto | 0 |
| 2544 | Seq. ID No. 5323 | Seq. ID No. 5324 | Cyto | 0 |
| 2545 | Seq. ID No. 5325 | Seq. ID No. 5326 | Cyto | |
| 2548a | Seq. ID No. 5333 | Seq. ID No. 5334 | Cyto | 0 |
| 2548b | Seq. ID No. 5335 | Seq. ID No. 5336 | Cyto | 0 |
| 2549 | Seq. ID No. 5337 | Seq. ID No. 5338 | Cyto | 0 |
| 2550b | Seq. ID No. 5339 | Seq. ID No. 5340 | Cyto | |
| 2550e | Seq. ID No. 5343 | Seq. ID No. 5344 | IM | |
| 2552 | Seq. ID No. 5345 | Seq. ID No. 5346 | Cyto | 0 |
| 2553 | Seq. ID No. 5347 | Seq. ID No. 5348 | Cyto | |
| 2554a | Seq. ID No. 5349 | Seq. ID No. 5350 | Cyto | |
| 2554b | Seq. ID No. 5351 | Seq. ID No. 5352 | Cyto | |
| 2555a | Seq. ID No. 5353 | Seq. ID No. 5354 | Cyto | |
| 2555b | Seq. ID No. 5355 | Seq. ID No. 5356 | Cyto | 0 |
| 2555c | Seq. ID No. 5357 | Seq. ID No. 5358 | Cyto | 0 |
| 2555d | Seq. ID No. 5359 | Seq. ID No. 5360 | Cyto | 0 |
| 2558a | Seq. ID No. 5361 | Seq. ID No. 5362 | Cyto | |
| 2558b | Seq. ID No. 5363 | Seq. ID No. 5364 | Cyto | |
| 2559 | Seq. ID No. 5365 | Seq. ID No. 5366 | Cyto | |
| 2561 | Seq. ID No. 5367 | Seq. ID No. 5368 | Cyto | 1 |
| 2562a | Seq. ID No. 5369 | Seq. ID No. 5370 | Cyto | |
| 2562c | Seq. ID No. 5371 | Seq. ID No. 5372 | Cyto | |
| 2563 | Seq. ID No. 5373 | Seq. ID No. 5374 | Cyto | |
| 2564a | Seq. ID No. 5375 | Seq. ID No. 5376 | Cyto | |
| 2564d | Seq. ID No. 5381 | Seq. ID No. 5382 | IM | 1 |
| 2566 | Seq. ID No. 5383 | Seq. ID No. 5384 | IM | |
| 2568 | Seq. ID No. 5389 | Seq. ID No. 5390 | IM | 2 |
| 2572 | Seq. ID No. 5391 | Seq. ID No. 5392 | IM | 0 |
| 2573 | Seq. ID No. 5393 | Seq. ID No. 5394 | Cyto | |
| 2575 | Seq. ID No. 5395 | Seq. ID No. 5396 | Cyto | |
| 2576 | Seq. ID No. 5397 | Seq. ID No. 5398 | Cyto | |
| 2578 | Seq. ID No. 5399 | Seq. ID No. 5400 | Cyto | |
| 2579b | Seq. ID No. 5401 | Seq. ID No. 5402 | IM | |
| 2581 | Seq. ID No. 5403 | Seq. ID No. 5404 | IM | 1 |
| 2582b | Seq. ID No. 5407 | Seq. ID No. 5408 | IM | 4 |
| 2582c | Seq. ID No. 5409 | Seq. ID No. 5410 | IM | 4 |
| 2585 | Seq. ID No. 5411 | Seq. ID No. 5412 | IM | |
| 2588 | Seq. ID No. 5417 | Seq. ID No. 5418 | Cyto | 0 |
| 2589 | Seq. ID No. 5419 | Seq. ID No. 5420 | Cyto | 1 |
| 2591 | Seq. ID No. 5421 | Seq. ID No. 5422 | Cyto | 0 |
| 2592 | Seq. ID No. 5423 | Seq. ID No. 5424 | Cyto | |
| 2593a | Seq. ID No. 5425 | Seq. ID No. 5426 | Cyto | 0 |
| 2593b | Seq. ID No. 5427 | Seq. ID No. 5428 | Cyto | 0 |
| 2594a | Seq. ID No. 5429 | Seq. ID No. 5430 | Cyto | |
| 2594b | Seq. ID No. 5431 | Seq. ID No. 5432 | Cyto | |
| 2597a | Seq. ID No. 5435 | Seq. ID No. 5436 | Cyto | |
| 2597b | Seq. ID No. 5437 | Seq. ID No. 5438 | Cyto | |
| 2597c | Seq. ID No. 5439 | Seq. ID No. 5440 | Cyto | |
| 2598 | Seq. ID No. 5441 | Seq. ID No. 5442 | Cyto | 0 |
| 2602 | Seq. ID No. 5443 | Seq. ID No. 5444 | Cyto | 0 |
| 2605a | Seq. ID No. 5445 | Seq. ID No. 5446 | Cyto | |
| 2605b | Seq. ID No. 5447 | Seq. ID No. 5448 | Cyto | |
| 2606 | Seq. ID No. 5449 | Seq. ID No. 5450 | Cyto | |
| 2608 | Seq. ID No. 5451 | Seq. ID No. 5452 | Cyto | |
| 2611 | Seq. ID No. 5455 | Seq. ID No. 5456 | Cyto | |
| 2613 | Seq. ID No. 5457 | Seq. ID No. 5458 | Cyto | 0 |
| 2616e | Seq. ID No. 5467 | Seq. ID No. 5468 | IM | 5 |
| 2616f | Seq. ID No. 5469 | Seq. ID No. 5470 | IM | 6 |
| 2616g | Seq. ID No. 5471 | Seq. ID No. 5472 | IM | 7 |
| 2616h | Seq. ID No. 5473 | Seq. ID No. 5474 | IM | 7 |
| 2617a | Seq. ID No. 5475 | Seq. ID No. 5476 | IM | |
| 2617b | Seq. ID No. 5477 | Seq. ID No. 5478 | IM | |
| 2618a | Seq. ID No. 5479 | Seq. ID No. 5480 | IM | |
| 2618b | Seq. ID No. 5481 | Seq. ID No. 5482 | IM | |
| 2619 | Seq. ID No. 5483 | Seq. ID No. 5484 | Cyto | |
| 2620 | Seq. ID No. 5485 | Seq. ID No. 5486 | IM | 0 |
| 2621a | Seq. ID No. 5487 | Seq. ID No. 5488 | IM | 1 |
| 2623a | Seq. ID No. 5489 | Seq. ID No. 5490 | Cyto | |
| 2624a | Seq. ID No. 5493 | Seq. ID No. 5494 | Cyto | 0 |
| 2624b | Seq. ID No. 5495 | Seq. ID No. 5496 | Cyto | 0 |
| 2625b | Seq. ID No. 5499 | Seq. ID No. 5500 | IM | 3 |
| 2626a | Seq. ID No. 5501 | Seq. ID No. 5502 | Cyto | |
| 2626b | Seq. ID No. 5503 | Seq. ID No. 5504 | Cyto | |
| 2627a | Seq. ID No. 5505 | Seq. ID No. 5506 | Cyto | |
| 2627b | Seq. ID No. 5507 | Seq. ID No. 5508 | Cyto | |
| 2629a | Seq. ID No. 5511 | Seq. ID No. 5512 | Cyto | |
| 2633a | Seq. ID No. 5519 | Seq. ID No. 5520 | Cyto | |
| 2633b | Seq. ID No. 5521 | Seq. ID No. 5522 | Cyto | |
| 2633c | Seq. ID No. 5523 | Seq. ID No. 5524 | Cyto | |
| 2633e | Seq. ID No. 5527 | Seq. ID No. 5528 | IM | 1 |
| 2643a | Seq. ID No. 5535 | Seq. ID No. 5536 | Cyto | 1 |
| 2643b | Seq. ID No. 5537 | Seq. ID No. 5538 | IM | 2 |
| 2645 | Seq. ID No. 5539 | Seq. ID No. 5540 | Cyto | |
| 2647 | Seq. ID No. 5541 | Seq. ID No. 5542 | Cyto | |
| 2649 | Seq. ID No. 5547 | Seq. ID No. 5548 | IM | 1 |
| 2650 | Seq. ID No. 5549 | Seq. ID No. 5550 | IM | 3 |
| 2652a | Seq. ID No. 5551 | Seq. ID No. 5552 | Cyto | |
| 2652b | Seq. ID No. 5553 | Seq. ID No. 5554 | Cyto | |
| 2655a | Seq. ID No. 5555 | Seq. ID No. 5556 | Cyto | |
| 2655b | Seq. ID No. 5557 | Seq. ID No. 5558 | IM | 1 |
| 2656 | Seq. ID No. 5559 | Seq. ID No. 5560 | Cyto | 0 |
| 2658a | Seq. ID No. 5561 | Seq. ID No. 5562 | Cyto | 0 |
| 2658b | Seq. ID No. 5563 | Seq. ID No. 5564 | Cyto | 0 |
| 2659a | Seq. ID No. 5565 | Seq. ID No. 5566 | IM | 3 |
| 2659b | Seq. ID No. 5567 | Seq. ID No. 5568 | IM | 4 |
| 2662 | Seq. ID No. 5571 | Seq. ID No. 5572 | Cyto | |
| 2664a | Seq. ID No. 5573 | Seq. ID No. 5574 | Cyto | |
| 2664b | Seq. ID No. 5575 | Seq. ID No. 5576 | Cyto | |
| 2665 | Seq. ID No. 5577 | Seq. ID No. 5578 | IM | 1 |
| 2666a | Seq. ID No. 5579 | Seq. ID No. 5580 | Cyto | 0 |
| 2666b | Seq. ID No. 5581 | Seq. ID No. 5582 | Cyto | 0 |
| 2667a | Seq. ID No. 5583 | Seq. ID No. 5584 | IM | |
| 2667b | Seq. ID No. 5585 | Seq. ID No. 5586 | IM | |
| 2674b | Seq. ID No. 5599 | Seq. ID No. 5600 | IM | 11 |
| 2676 | Seq. ID No. 5601 | Seq. ID No. 5602 | Cyto | 0 |
| 2677a | Seq. ID No. 5603 | Seq. ID No. 5604 | IM | 1 |
| 2677b | Seq. ID No. 5605 | Seq. ID No. 5606 | IM | 1 |
| 2677c | Seq. ID No. 5607 | Seq. ID No. 5608 | IM | 1 |
| 2679c | Seq. ID No. 5613 | Seq. ID No. 5614 | IM | 5 |
| 2679d | Seq. ID No. 5615 | Seq. ID No. 5616 | IM | 5 |
| 2680a | Seq. ID No. 5617 | Seq. ID No. 5618 | Cyto | |
| 2680b | Seq. ID No. 5619 | Seq. ID No. 5620 | Cyto | |
| 2684a | Seq. ID No. 5627 | Seq. ID No. 5628 | IM | 0 |
| 2684b | Seq. ID No. 5629 | Seq. ID No. 5630 | IM | 0 |
| 2686 | Seq. ID No. 5631 | Seq. ID No. 5632 | Cyto | 0 |
| 2688b | Seq. ID No. 5635 | Seq. ID No. 5636 | IM | 4 |
| 2690b | Seq. ID No. 5645 | Seq. ID No. 5646 | IM | 1 |
| 2690c | Seq. ID No. 5647 | Seq. ID No. 5648 | IM | 1 |
| 2693 | Seq. ID No. 5651 | Seq. ID No. 5652 | Cyto | |
| 2694a | Seq. ID No. 5653 | Seq. ID No. 5654 | Cyto | 0 |
| 2694b | Seq. ID No. 5655 | Seq. ID No. 5656 | Cyto | 0 |
| 2695 | Seq. ID No. 5657 | Seq. ID No. 5658 | Cyto | 0 |
| 2696b | Seq. ID No. 5661 | Seq. ID No. 5662 | IM | 8 |
| 2705 | Seq. ID No. 5663 | Seq. ID No. 5664 | Cyto | |
| 2709 | Seq. ID No. 5665 | Seq. ID No. 5666 | Cyto | |
| 2712 | Seq. ID No. 5669 | Seq. ID No. 5670 | IM | 1 |
| 2713 | Seq. ID No. 5671 | Seq. ID No. 5672 | Cyto | |
| 2714 | Seq. ID No. 5673 | Seq. ID No. 5674 | Cyto | |
| 2716a | Seq. ID No. 5675 | Seq. ID No. 5676 | Cyto | |
| 2716b | Seq. ID No. 5677 | Seq. ID No. 5678 | Cyto | |
| 2716c | Seq. ID No. 5679 | Seq. ID No. 5680 | Cyto | |
| 2721a | Seq. ID No. 5691 | Seq. ID No. 5692 | Cyto | |
| 2721b | Seq. ID No. 5693 | Seq. ID No. 5694 | IM | 1 |
| 2722a | Seq. ID No. 5695 | Seq. ID No. 5696 | Cyto | 1 |
| 2722b | Seq. ID No. 5697 | Seq. ID No. 5698 | Cyto | 1 |
| 2724 | Seq. ID No. 5701 | Seq. ID No. 5702 | Cyto | |
| 2726 | Seq. ID No. 5707 | Seq. ID No. 5708 | Cyto | |
| 2731a | Seq. ID No. 5713 | Seq. ID No. 5714 | Cyto | |
| 2731b | Seq. ID No. 5715 | Seq. ID No. 5716 | Cyto | |
| 2732 | Seq. ID No. 5717 | Seq. ID No. 5718 | Cyto | |
| 2734b | Seq. ID No. 5721 | Seq. ID No. 5722 | IM | 4 |
| 2734c | Seq. ID No. 5723 | Seq. ID No. 5724 | IM | 5 |
| 2735a | Seq. ID No. 5725 | Seq. ID No. 5726 | Cyto | 2 |
| 2735b | Seq. ID No. 5727 | Seq. ID No. 5728 | Cyto | 2 |
| 2738b | Seq. ID No. 5731 | Seq. ID No. 5732 | IM | 11 |
| 2742a | Seq. ID No. 5737 | Seq. ID No. 5738 | Cyto | |
| 2743a | Seq. ID No. 5741 | Seq. ID No. 5742 | Cyto | 1 |
| 2743b | Seq. ID No. 5743 | Seq. ID No. 5744 | Cyto | 1 |
| 2744 | Seq. ID No. 5745 | Seq. ID No. 5746 | IM | |
| 2745a | Seq. ID No. 5747 | Seq. ID No. 5748 | Cyto | 1 |
| 2745b | Seq. ID No. 5749 | Seq. ID No. 5750 | Cyto | 1 |
| 2746 | Seq. ID No. 5751 | Seq. ID No. 5752 | Cyto | |
| 2747a | Seq. ID No. 5753 | Seq. ID No. 5754 | Cyto | 0 |
| 2747b | Seq. ID No. 5755 | Seq. ID No. 5756 | Cyto | 0 |
| 2748a | Seq. ID No. 5757 | Seq. ID No. 5758 | IM | 10 |
| 2748b | Seq. ID No. 5759 | Seq. ID No. 5760 | IM | 10 |
| 2748c | Seq. ID No. 5761 | Seq. ID No. 5762 | IM | 10 |
| 2749d | Seq. ID No. 5769 | Seq. ID No. 5770 | IM | 9 |
| 2750a | Seq. ID No. 5773 | Seq. ID No. 5774 | Cyto | |
| 2750b | Seq. ID No. 5775 | Seq. ID No. 5776 | Cyto | |
| 2752 | Seq. ID No. 5777 | Seq. ID No. 5778 | Cyto | 0 |
| 2753 | Seq. ID No. 5779 | Seq. ID No. 5780 | Cyto | |
| 2757a | Seq. ID No. 5781 | Seq. ID No. 5782 | IM | 4 |
| 2758b | Seq. ID No. 5787 | Seq. ID No. 5788 | Cyto | 0 |
| 2758c | Seq. ID No. 5789 | Seq. ID No. 5790 | Cyto | 0 |
| 2759a | Seq. ID No. 5793 | Seq. ID No. 5794 | Cyto | 0 |
| 2759b | Seq. ID No. 5795 | Seq. ID No. 5796 | Cyto | 0 |
| 2760b | Seq. ID No. 5799 | Seq. ID No. 5800 | IM | 1 |
| 2760c | Seq. ID No. 5801 | Seq. ID No. 5802 | IM | 1 |
| 2762 | Seq. ID No. 5803 | Seq. ID No. 5804 | Cyto | 0 |
| 2763a | Seq. ID No. 5805 | Seq. ID No. 5806 | IM | 2 |
| 2763b | Seq. ID No. 5807 | Seq. ID No. 5808 | IM | 2 |
| 2763c | Seq. ID No. 5809 | Seq. ID No. 5810 | IM | 2 |
| 2764 | Seq. ID No. 5811 | Seq. ID No. 5812 | IM | |
| 2765b | Seq. ID No. 5813 | Seq. ID No. 5814 | Cyto | 1 |
| 2765c | Seq. ID No. 5815 | Seq. ID No. 5816 | Cyto | 1 |
| 2766a | Seq. ID No. 5817 | Seq. ID No. 5818 | IM | 0 |
| 2766b | Seq. ID No. 5819 | Seq. ID No. 5820 | IM | 0 |
| 2768a | Seq. ID No. 5821 | Seq. ID No. 5822 | IM | 0 |
| 2768b | Seq. ID No. 5823 | Seq. ID No. 5824 | IM | 0 |
| 2768c | Seq. ID No. 5825 | Seq. ID No. 5826 | IM | 0 |
| 2769a | Seq. ID No. 5827 | Seq. ID No. 5828 | IM | 0 |
| 2771a | Seq. ID No. 5831 | Seq. ID No. 5832 | Cyto | |
| 2771b | Seq. ID No. 5833 | Seq. ID No. 5834 | Cyto | |
| 2773 | Seq. ID No. 5835 | Seq. ID No. 5836 | IM | 1 |
| 2774a | Seq. ID No. 5837 | Seq. ID No. 5838 | IM | 2 |
| 2774b | Seq. ID No. 5839 | Seq. ID No. 5840 | IM | 2 |
| 2774c | Seq. ID No. 5841 | Seq. ID No. 5842 | IM | 2 |
| 2776a | Seq. ID No. 5843 | Seq. ID No. 5844 | Cyto | 1 |
| 2776b | Seq. ID No. 5845 | Seq. ID No. 5846 | Cyto | 1 |
| 2777 | Seq. ID No. 5847 | Seq. ID No. 5848 | Cyto | |
| 2778 | Seq. ID No. 5849 | Seq. ID No. 5850 | Cyto | |
| 2779 | Seq. ID No. 5851 | Seq. ID No. 5852 | IM | 1 |
| 2780 | Seq. ID No. 5853 | Seq. ID No. 5854 | Cyto | |
| 2784 | Seq. ID No. 5855 | Seq. ID No. 5856 | Cyto | |
| 2785a | Seq. ID No. 5857 | Seq. ID No. 5858 | Cyto | 1 |
| 2785b | Seq. ID No. 5859 | Seq. ID No. 5860 | Cyto | 1 |
| 2786a | Seq. ID No. 5861 | Seq. ID No. 5862 | IM | 1 |
| 2786b | Seq. ID No. 5863 | Seq. ID No. 5864 | IM | 1 |
| 2786c | Seq. ID No. 5865 | Seq. ID No. 5866 | IM | 1 |
| 2787a | Seq. ID No. 5867 | Seq. ID No. 5868 | Cyto | 1 |
| 2787b | Seq. ID No. 5869 | Seq. ID No. 5870 | Cyto | 1 |
| 2787c | Seq. ID No. 5871 | Seq. ID No. 5872 | Cyto | 1 |
| 2788b | Seq. ID No. 5873 | Seq. ID No. 5874 | Cyto | 1 |
| 2789 | Seq. ID No. 5875 | Seq. ID No. 5876 | Cyto | 0 |
| 2790 | Seq. ID No. 5877 | Seq. ID No. 5878 | Cyto | |
| 2793 | Seq. ID No. 5881 | Seq. ID No. 5882 | Cyto | |
| 2798 | Seq. ID No. 5887 | Seq. ID No. 5888 | IM | 2 |
| 2799b | Seq. ID No. 5891 | Seq. ID No. 5892 | IM | 1 |
| 2804 | Seq. ID No. 5897 | Seq. ID No. 5898 | IM | 0 |
| 2809 | Seq. ID No. 5903 | Seq. ID No. 5904 | Cyto | |
| 2810b | Seq. ID No. 5907 | Seq. ID No. 5908 | IM | 3 |
| 2810d | Seq. ID No. 5911 | Seq. ID No. 5912 | IM | 4 |
| 2812 | Seq. ID No. 5913 | Seq. ID No. 5914 | Cyto | |
| 2814 | Seq. ID No. 5915 | Seq. ID No. 5916 | IM | 1 |
| 2815 | Seq. ID No. 5917 | Seq. ID No. 5918 | Cyto | |
| 2816 | Seq. ID No. 5919 | Seq. ID No. 5920 | Cyto | |
| 2818c | Seq. ID No. 5925 | Seq. ID No. 5926 | IM | 5 |
| 2819 | Seq. ID No. 5931 | Seq. ID No. 5932 | Cyto | |
| 2820 | Seq. ID No. 5933 | Seq. ID No. 5934 | Cyto | |
| 2821a | Seq. ID No. 5935 | Seq. ID No. 5936 | Cyto | |
| 2821b | Seq. ID No. 5937 | Seq. ID No. 5938 | Cyto | |
| 2822b | Seq. ID No. 5941 | Seq. ID No. 5942 | IM | 11 |
| 2823 | Seq. ID No. 5943 | Seq. ID No. 5944 | Cyto | 0 |
| 2824a | Seq. ID No. 5945 | Seq. ID No. 5946 | Cyto | 1 |
| 2824b | Seq. ID No. 5947 | Seq. ID No. 5948 | Cyto | 1 |
| 2829a | Seq. ID No. 5949 | Seq. ID No. 5950 | Cyto | |
| 2829b | Seq. ID No. 5951 | Seq. ID No. 5952 | Cyto | |
| 2831b | Seq. ID No. 5953 | Seq. ID No. 5954 | Cyto | |
| 2832 | Seq. ID No. 5955 | Seq. ID No. 5956 | Cyto | |
| 2833 | Seq. ID No. 5957 | Seq. ID No. 5958 | Cyto | |
| 2834a | Seq. ID No. 5959 | Seq. ID No. 5960 | Cyto | 0 |
| 2834b | Seq. ID No. 5961 | Seq. ID No. 5962 | Cyto | 0 |
| 2837a | Seq. ID No. 5963 | Seq. ID No. 5964 | IM | 2 |
| 2837b | Seq. ID No. 5965 | Seq. ID No. 5966 | IM | 3 |
| 2842a | Seq. ID No. 5967 | Seq. ID No. 5968 | Cyto | |
| 2842b | Seq. ID No. 5969 | Seq. ID No. 5970 | Cyto | |
| 2843a | Seq. ID No. 5971 | Seq. ID No. 5972 | Cyto | |
| 2843b | Seq. ID No. 5973 | Seq. ID No. 5974 | Cyto | |
| 2845 | Seq. ID No. 5975 | Seq. ID No. 5976 | Cyto | |
| 2846a | Seq. ID No. 5977 | Seq. ID No. 5978 | Cyto | |
| 2846b | Seq. ID No. 5979 | Seq. ID No. 5980 | Peri | |
| 2848 | Seq. ID No. 5981 | Seq. ID No. 5982 | Cyto | |
| 2850 | Seq. ID No. 5983 | Seq. ID No. 5984 | Cyto | |
| 2851 | Seq. ID No. 5985 | Seq. ID No. 5986 | Cyto | 0 |
| 2852 | Seq. ID No. 5987 | Seq. ID No. 5988 | IM | 1 |
| 2853 | Seq. ID No. 5989 | Seq. ID No. 5990 | Cyto | |
| 2854a | Seq. ID No. 5991 | Seq. ID No. 5992 | IM | |
| 2854b | Seq. ID No. 5993 | Seq. ID No. 5994 | IM | |
| 2855 | Seq. ID No. 5995 | Seq. ID No. 5996 | Cyto | |
| 2856a | Seq. ID No. 5997 | Seq. ID No. 5998 | IM | 8 |
| 2856b | Seq. ID No. 5999 | Seq. ID No. 6000 | IM | 8 |
| 2856c | Seq. ID No. 6001 | Seq. ID No. 6002 | IM | 8 |
| 2857 | Seq. ID No. 6003 | Seq. ID No. 6004 | Cyto | |
| 2859 | Seq. ID No. 6005 | Seq. ID No. 6006 | Cyto | |
| 2860a | Seq. ID No. 6007 | Seq. ID No. 6008 | Cyto | 0 |
| 2860b | Seq. ID No. 6009 | Seq. ID No. 6010 | Cyto | 0 |
| 2860c | Seq. ID No. 6011 | Seq. ID No. 6012 | Cyto | 0 |
| 2862 | Seq. ID No. 6013 | Seq. ID No. 6014 | Cyto | 2 |
| 2863 | Seq. ID No. 6015 | Seq. ID No. 6016 | Cyto | |
| 2864 | Seq. ID No. 6017 | Seq. ID No. 6018 | Cyto | 2 |
| 2868a | Seq. ID No. 6019 | Seq. ID No. 6020 | Cyto | |
| 2868b | Seq. ID No. 6021 | Seq. ID No. 6022 | IM | |
| 2871 | Seq. ID No. 6029 | Seq. ID No. 6030 | Cyto | |
| 2872 | Seq. ID No. 6031 | Seq. ID No. 6032 | Cyto | |
| 2875 | Seq. ID No. 6033 | Seq. ID No. 6034 | Cyto | 0 |
| 2877 | Seq. ID No. 6035 | Seq. ID No. 6036 | Cyto | |
| 2879a | Seq. ID No. 6037 | Seq. ID No. 6038 | Cyto | 0 |
| 2879b | Seq. ID No. 6039 | Seq. ID No. 6040 | Cyto | 0 |
| 2879c | Seq. ID No. 6041 | Seq. ID No. 6042 | Cyto | 0 |
| 2882 | Seq. ID No. 6045 | Seq. ID No. 6046 | IM | 2 |
| 2883b | Seq. ID No. 6049 | Seq. ID No. 6050 | IM | 7 |
| 2883c | Seq. ID No. 6051 | Seq. ID No. 6052 | IM | 7 |
| 2886a | Seq. ID No. 6055 | Seq. ID No. 6056 | Cyto | 0 |
| 2886b | Seq. ID No. 6057 | Seq. ID No. 6058 | Cyto | 0 |
| 2887b | Seq. ID No. 6061 | Seq. ID No. 6062 | IM | 2 |
| 2890 | Seq. ID No. 6065 | Seq. ID No. 6066 | IM | 1 |
| 2891b | Seq. ID No. 6069 | Seq. ID No. 6070 | Cyto | 1 |
| 2891c | Seq. ID No. 6071 | Seq. ID No. 6072 | Cyto | 1 |
| 2893b | Seq. ID No. 6073 | Seq. ID No. 6074 | IM | 1 |
| 2894a | Seq. ID No. 6075 | Seq. ID No. 6076 | Cyto | |
| 2894b | Seq. ID No. 6077 | Seq. ID No. 6078 | Cyto | |
| 2894c | Seq. ID No. 6079 | Seq. ID No. 6080 | Cyto | |
| 2895 | Seq. ID No. 6081 | Seq. ID No. 6082 | IM | |
| 2897a | Seq. ID No. 6083 | Seq. ID No. 6084 | Cyto | |
| 2897b | Seq. ID No. 6085 | Seq. ID No. 6086 | Cyto | |
| 2901b | Seq. ID No. 6093 | Seq. ID No. 6094 | IM | 2 |
| 2904 | Seq. ID No. 6097 | Seq. ID No. 6098 | Cyto | |
| 2905a | Seq. ID No. 6099 | Seq. ID No. 6100 | Cyto | |
| 2905b | Seq. ID No. 6101 | Seq. ID No. 6102 | Cyto | 0 |
| 2909a | Seq. ID No. 6105 | Seq. ID No. 6106 | Cyto | |
| 2912 | Seq. ID No. 6113 | Seq. ID No. 6114 | Cyto | |
| 2915a | Seq. ID No. 6119 | Seq. ID No. 6120 | Cyto | |
| 2915b | Seq. ID No. 6121 | Seq. ID No. 6122 | Cyto | |
| 2915c | Seq. ID No. 6123 | Seq. ID No. 6124 | Cyto | |
| 2916 | Seq. ID No. 6125 | Seq. ID No. 6126 | Cyto | |
| 2917 | Seq. ID No. 6127 | Seq. ID No. 6128 | OM | 1 |
| 2918 | Seq. ID No. 6129 | Seq. ID No. 6130 | Cyto | 0 |
| 2920 | Seq. ID No. 6131 | Seq. ID No. 6132 | IM | 2 |
| 2921 | Seq. ID No. 6133 | Seq. ID No. 6134 | IM | 0 |
| 2923b | Seq. ID No. 6139 | Seq. ID No. 6140 | Cyto | |
| 2923c | Seq. ID No. 6141 | Seq. ID No. 6142 | Cyto | |
| 2925a | Seq. ID No. 6143 | Seq. ID No. 6144 | Cyto | |
| 2925b | Seq. ID No. 6145 | Seq. ID No. 6146 | Cyto | |
| 2926a | Seq. ID No. 6147 | Seq. ID No. 6148 | Cyto | |
| 2926b | Seq. ID No. 6149 | Seq. ID No. 6150 | Cyto | |
| 2926c | Seq. ID No. 6151 | Seq. ID No. 6152 | Cyto | |
| 2928a | Seq. ID No. 6153 | Seq. ID No. 6154 | Cyto | |
| 2928b | Seq. ID No. 6155 | Seq. ID No. 6156 | Cyto | |
| 2928c | Seq. ID No. 6157 | Seq. ID No. 6158 | Cyto | |
| 2933a | Seq. ID No. 6167 | Seq. ID No. 6168 | Cyto | |
| 2934 | Seq. ID No. 6171 | Seq. ID No. 6172 | Cyto | 1 |
| 2935a | Seq. ID No. 6173 | Seq. ID No. 6174 | Cyto | |
| 2935b | Seq. ID No. 6175 | Seq. ID No. 6176 | Cyto | 0 |
| 2936 | Seq. ID No. 6177 | Seq. ID No. 6178 | IM | 2 |
| 2939a | Seq. ID No. 6179 | Seq. ID No. 6180 | Cyto | |
| 2939b | Seq. ID No. 6181 | Seq. ID No. 6182 | Cyto | |
| 2943c | Seq. ID No. 6193 | Seq. ID No. 6194 | IM | 6 |
| 2943d | Seq. ID No. 6195 | Seq. ID No. 6196 | IM | 6 |
| 2945b | Seq. ID No. 6199 | Seq. ID No. 6200 | IM | 0 |
| 2947 | Seq. ID No. 6201 | Seq. ID No. 6202 | Cyto | |
| 2948c | Seq. ID No. 6207 | Seq. ID No. 6208 | IM | 6 |
| 2950a | Seq. ID No. 6211 | Seq. ID No. 6212 | Cyto | |
| 2950b | Seq. ID No. 6213 | Seq. ID No. 6214 | Cyto | |
| 2951 | Seq. ID No. 6215 | Seq. ID No. 6216 | Cyto | |
| 2954 | Seq. ID No. 6219 | Seq. ID No. 6220 | IM | 7 |
| 2955f | Seq. ID No. 6231 | Seq. ID No. 6232 | IM | 12 |
| 2955g | Seq. ID No. 6233 | Seq. ID No. 6234 | IM | 12 |
| 2958b | Seq. ID No. 6239 | Seq. ID No. 6240 | IM | 2 |
| 2962a | Seq. ID No. 6247 | Seq. ID No. 6248 | Cyto | |
| 2963 | Seq. ID No. 6251 | Seq. ID No. 6252 | Cyto | |
| 2965b | Seq. ID No. 6255 | Seq. ID No. 6256 | IM | 2 |
| 2965c | Seq. ID No. 6257 | Seq. ID No. 6258 | IM | 2 |
| 2967 | Seq. ID No. 6261 | Seq. ID No. 6262 | Cyto | |
| 2971a | Seq. ID No. 6269 | Seq. ID No. 6270 | Cyto | 0 |
| 2971b | Seq. ID No. 6271 | Seq. ID No. 6272 | Cyto | 0 |
| 2975 | Seq. ID No. 6277 | Seq. ID No. 6278 | Cyto | 1 |
| 2977a | Seq. ID No. 6279 | Seq. ID No. 6280 | IM | 4 |
| 2977b | Seq. ID No. 6281 | Seq. ID No. 6282 | IM | 4 |
| 2979 | Seq. ID No. 6287 | Seq. ID No. 6288 | IM | 4 |
| 2980b | Seq. ID No. 6291 | Seq. ID No. 6292 | IM | 5 |
| 2980c | Seq. ID No. 6293 | Seq. ID No. 6294 | IM | 5 |
| 2980d | Seq. ID No. 6295 | Seq. ID No. 6296 | IM | 5 |
| 2980e | Seq. ID No. 6297 | Seq. ID No. 6298 | IM | 5 |
| 2984 | Seq. ID No. 6299 | Seq. ID No. 6300 | Cyto | 0 |
| 2986c | Seq. ID No. 6305 | Seq. ID No. 6306 | IM | 7 |
| 2988a | Seq. ID No. 6307 | Seq. ID No. 6308 | Cyto | 1 |
| 2988b | Seq. ID No. 6309 | Seq. ID No. 6310 | Cyto | 1 |
| 2988c | Seq. ID No. 6311 | Seq. ID No. 6312 | Cyto | 1 |
| 2989 | Seq. ID No. 6313 | Seq. ID No. 6314 | Cyto | |
| 2990 | Seq. ID No. 6315 | Seq. ID No. 6316 | Cyto | 0 |
| 2991a | Seq. ID No. 6317 | Seq. ID No. 6318 | IM | |
| 2991b | Seq. ID No. 6319 | Seq. ID No. 6320 | Cyto | 0 |
| 2991c | Seq. ID No. 6321 | Seq. ID No. 6322 | Cyto | 0 |
| 2992 | Seq. ID No. 6323 | Seq. ID No. 6324 | IM | 4 |
| 2995a | Seq. ID No. 6329 | Seq. ID No. 6330 | Cyto | 1 |
| 2995b | Seq. ID No. 6331 | Seq. ID No. 6332 | Cyto | 1 |
| 2997 | Seq. ID No. 6333 | Seq. ID No. 6334 | Cyto | |
| 2998a | Seq. ID No. 6335 | Seq. ID No. 6336 | Cyto | |
| 2998b | Seq. ID No. 6337 | Seq. ID No. 6338 | Cyto | |
| 2999 | Seq. ID No. 6339 | Seq. ID No. 6340 | Cyto | |
| 3001 | Seq. ID No. 6341 | Seq. ID No. 6342 | Cyto | |
| 3003 | Seq. ID No. 6343 | Seq. ID No. 6344 | Cyto | |
| 3005 | Seq. ID No. 6345 | Seq. ID No. 6346 | Cyto | |
| 3008a | Seq. ID No. 6347 | Seq. ID No. 6348 | Cyto | |
| 3008b | Seq. ID No. 6349 | Seq. ID No. 6350 | Cyto | |
| 3008c | Seq. ID No. 6351 | Seq. ID No. 6352 | Cyto | |
| 3008d | Seq. ID No. 6353 | Seq. ID No. 6354 | Cyto | |
| 3010a | Seq. ID No. 6355 | Seq. ID No. 6356 | Cyto | |
| 3010b | Seq. ID No. 6357 | Seq. ID No. 6358 | Cyto | |
| 3012 | Seq. ID No. 6359 | Seq. ID No. 6360 | Cyto | |
| 3015 | Seq. ID No. 6361 | Seq. ID No. 6362 | IM | 7 |
| 3016c | Seq. ID No. 6367 | Seq. ID No. 6368 | IM | 7 |
| 3017 | Seq. ID No. 6369 | Seq. ID No. 6370 | Cyto | |
| 3018a | Seq. ID No. 6371 | Seq. ID No. 6372 | Cyto | |
| 3018b | Seq. ID No. 6373 | Seq. ID No. 6374 | Cyto | |
| 3020 | Seq. ID No. 6375 | Seq. ID No. 6376 | IM | |
| 3022 | Seq. ID No. 6379 | Seq. ID No. 6380 | Cyto | |
| 3024b | Seq. ID No. 6385 | Seq. ID No. 6386 | Cyto | 1 |
| 3026a | Seq. ID No. 6387 | Seq. ID No. 6388 | Cyto | |
| 3026b | Seq. ID No. 6389 | Seq. ID No. 6390 | Cyto | |
| 3028a | Seq. ID No. 6391 | Seq. ID No. 6392 | Cyto | |
| 3028b | Seq. ID No. 6393 | Seq. ID No. 6394 | Cyto | |
| 3028c | Seq. ID No. 6395 | Seq. ID No. 6396 | Cyto | |
| 3029a | Seq. ID No. 6397 | Seq. ID No. 6398 | Cyto | |
| 3029b | Seq. ID No. 6399 | Seq. ID No. 6400 | Cyto | |
| 3031a | Seq. ID No. 6401 | Seq. ID No. 6402 | Cyto | |
| 3031b | Seq. ID No. 6403 | Seq. ID No. 6404 | Cyto | |
| 3032a | Seq. ID No. 6405 | Seq. ID No. 6406 | Cyto | |
| 3032b | Seq. ID No. 6407 | Seq. ID No. 6408 | Cyto | |
| 3033a | Seq. ID No. 6409 | Seq. ID No. 6410 | Cyto | |
| 3033b | Seq. ID No. 6411 | Seq. ID No. 6412 | Cyto | |
| 3035 | Seq. ID No. 6413 | Seq. ID No. 6414 | Cyto | 1 |
| 3037b | Seq. ID No. 6417 | Seq. ID No. 6418 | IM | 1 |
| 3038 | Seq. ID No. 6419 | Seq. ID No. 6420 | Cyto | |
| 3040a | Seq. ID No. 6421 | Seq. ID No. 6422 | Cyto | |
| 3040b | Seq. ID No. 6423 | Seq. ID No. 6424 | Cyto | |
| 3044 | Seq. ID No. 6433 | Seq. ID No. 6434 | IM | 1 |
| 3045a | Seq. ID No. 6435 | Seq. ID No. 6436 | Cyto | |
| 3045b | Seq. ID No. 6437 | Seq. ID No. 6438 | Cyto | |
| 3048a | Seq. ID No. 6439 | Seq. ID No. 6440 | Cyto | |
| 3049a | Seq. ID No. 6441 | Seq. ID No. 6442 | Cyto | |
| 3049b | Seq. ID No. 6443 | Seq. ID No. 6444 | Cyto | |
| 3049c | Seq. ID No. 6445 | Seq. ID No. 6446 | Cyto | |
| 3051a | Seq. ID No. 6447 | Seq. ID No. 6448 | Cyto | |
| 3051b | Seq. ID No. 6449 | Seq. ID No. 6450 | Cyto | |
| 3052 | Seq. ID No. 6451 | Seq. ID No. 6452 | Cyto | |
| 3054 | Seq. ID No. 6453 | Seq. ID No. 6454 | Cyto | |
| 3055a | Seq. ID No. 6455 | Seq. ID No. 6456 | Cyto | |
| 3055b | Seq. ID No. 6457 | Seq. ID No. 6458 | Cyto | |
| 3057 | Seq. ID No. 6459 | Seq. ID No. 6460 | Cyto | |
| 3059 | Seq. ID No. 6461 | Seq. ID No. 6462 | Cyto | |
| 3060 | Seq. ID No. 6463 | Seq. ID No. 6464 | IM | 5 |
| 3061 | Seq. ID No. 6465 | Seq. ID No. 6466 | Peri | 1 |
| 3062 | Seq. ID No. 6467 | Seq. ID No. 6468 | IM | 5 |
| 3063a | Seq. ID No. 6469 | Seq. ID No. 6470 | Cyto | 0 |
| 3063b | Seq. ID No. 6471 | Seq. ID No. 6472 | Cyto | 0 |
| 3065a | Seq. ID No. 6475 | Seq. ID No. 6476 | Cyto | |
| 3065b | Seq. ID No. 6477 | Seq. ID No. 6478 | Cyto | |
| 3067 | Seq. ID No. 6481 | Seq. ID No. 6482 | Cyto | |
| 3068 | Seq. ID No. 6483 | Seq. ID No. 6484 | Cyto | |
| 3069 | Seq. ID No. 6485 | Seq. ID No. 6486 | Cyto | |
| 3071 | Seq. ID No. 6487 | Seq. ID No. 6488 | Cyto | |
| 3072a | Seq. ID No. 6489 | Seq. ID No. 6490 | Cyto | |
| 3073 | Seq. ID No. 6491 | Seq. ID No. 6492 | Cyto | |
| 3074 | Seq. ID No. 6493 | Seq. ID No. 6494 | Cyto | |
| 3075 | Seq. ID No. 6495 | Seq. ID No. 6496 | Cyto | |
| 3077a | Seq. ID No. 6499 | Seq. ID No. 6500 | Cyto | |
| 3077b | Seq. ID No. 6501 | Seq. ID No. 6502 | Cyto | |
| 3077c | Seq. ID No. 6503 | Seq. ID No. 6504 | Cyto | |
| 3078 | Seq. ID No. 6505 | Seq. ID No. 6506 | Cyto | 1 |
| 3079 | Seq. ID No. 6507 | Seq. ID No. 6508 | IM | 1 |
| 3081a | Seq. ID No. 6509 | Seq. ID No. 6510 | Cyto | |
| 3081b | Seq. ID No. 6511 | Seq. ID No. 6512 | Cyto | |
| 3083c | Seq. ID No. 6513 | Seq. ID No. 6514 | Cyto | |
| 3083d | Seq. ID No. 6515 | Seq. ID No. 6516 | Cyto | |
| 3086 | Seq. ID No. 6519 | Seq. ID No. 6520 | Cyto | |
| 3087a | Seq. ID No. 6521 | Seq. ID No. 6522 | Cyto | |
| 3087c | Seq. ID No. 6523 | Seq. ID No. 6524 | Cyto | |
| 3088 | Seq. ID No. 6525 | Seq. ID No. 6526 | Cyto | |
| 3089a | Seq. ID No. 6527 | Seq. ID No. 6528 | Cyto | |
| 3089b | Seq. ID No. 6529 | Seq. ID No. 6530 | Cyto | |
| 3090 | Seq. ID No. 6531 | Seq. ID No. 6532 | Cyto | |
| 3091 | Seq. ID No. 6533 | Seq. ID No. 6534 | Cyto | |
| 3092a | Seq. ID No. 6535 | Seq. ID No. 6536 | Cyto | |
| 3092b | Seq. ID No. 6537 | Seq. ID No. 6538 | Cyto | |
| 3095 | Seq. ID No. 6539 | Seq. ID No. 6540 | IM | 1 |
| 3097 | Seq. ID No. 6545 | Seq. ID No. 6546 | Cyto | |
| 3098a | Seq. ID No. 6547 | Seq. ID No. 6548 | Cyto | 0 |
| 3098b | Seq. ID No. 6549 | Seq. ID No. 6550 | Cyto | 0 |
| 3098c | Seq. ID No. 6551 | Seq. ID No. 6552 | Cyto | 0 |
| 3099 | Seq. ID No. 6553 | Seq. ID No. 6554 | Cyto | |
| 3101 | Seq. ID No. 6559 | Seq. ID No. 6560 | Cyto | |
| 3102 | Seq. ID No. 6561 | Seq. ID No. 6562 | Peri | 2 |
| 3103b | Seq. ID No. 6563 | Seq. ID No. 6564 | Cyto | |
| 3104 | Seq. ID No. 6565 | Seq. ID No. 6566 | Cyto | |
| 3105 | Seq. ID No. 6567 | Seq. ID No. 6568 | Cyto | |
| 3106 | Seq. ID No. 6569 | Seq. ID No. 6570 | Cyto | |
| 3108a | Seq. ID No. 6575 | Seq. ID No. 6576 | Cyto | |
| 3108b | Seq. ID No. 6577 | Seq. ID No. 6578 | Peri | |
| 3109 | Seq. ID No. 6579 | Seq. ID No. 6580 | Cyto | |
| 3110b | Seq. ID No. 6583 | Seq. ID No. 6584 | IM | 2 |
| 3113 | Seq. ID No. 6585 | Seq. ID No. 6586 | IM | 1 |
| 3116 | Seq. ID No. 6587 | Seq. ID No. 6588 | Cyto | 1 |
| 3118a | Seq. ID No. 6589 | Seq. ID No. 6590 | Cyto | |
| 3118b | Seq. ID No. 6591 | Seq. ID No. 6592 | Cyto | |
| 3121 | Seq. ID No. 6605 | Seq. ID No. 6606 | Cyto | |
| 3122a | Seq. ID No. 6607 | Seq. ID No. 6608 | Cyto | 2 |
| 3122b | Seq. ID No. 6609 | Seq. ID No. 6610 | Cyto | 2 |
| 3123a | Seq. ID No. 6611 | Seq. ID No. 6612 | IM | 0 |
| 3123b | Seq. ID No. 6613 | Seq. ID No. 6614 | IM | 0 |
| 3124 | Seq. ID No. 6615 | Seq. ID No. 6616 | IM | 1 |
| 3126 | Seq. ID No. 6617 | Seq. ID No. 6618 | Cyto | 0 |
| 3129a | Seq. ID No. 6619 | Seq. ID No. 6620 | Cyto | 2 |
| 3129b | Seq. ID No. 6621 | Seq. ID No. 6622 | Cyto | 2 |
| 3131 | Seq. ID No. 6623 | Seq. ID No. 6624 | IM | 2 |
| 3134a | Seq. ID No. 6625 | Seq. ID No. 6626 | Cyto | 0 |
| 3134b | Seq. ID No. 6627 | Seq. ID No. 6628 | IM | 0 |
| 3138 | Seq. ID No. 6629 | Seq. ID No. 6630 | IM | 0 |
| 3141 | Seq. ID No. 6633 | Seq. ID No. 6634 | Cyto | 2 |
| 3145a | Seq. ID No. 6635 | Seq. ID No. 6636 | Cyto | 0 |
| 3147a | Seq. ID No. 6641 | Seq. ID No. 6642 | Cyto | |
| 3149a | Seq. ID No. 6647 | Seq. ID No. 6648 | Cyto | |

Listed in Table 7 are 287 ORFs whose Pfam results predict possible secreted, surface or membrane localization of proteins encoded by the ORFs.

TABLE 7
PFAM

| ORF | DNA SEQ ID | Protein SEQ ID | |
|---|---|---|---|
| Number | Number | Number | Pfam |
| 51c | Seq. ID No. 89 | Seq. ID No. 90 | Sodium: dicarboxylate symporter family |
| 80c | Seq. ID No. 145 | Seq. ID No. 146 | Formate/nitrite transporter |
| 85b | Seq. ID No. 155 | Seq. ID No. 156 | ABC transporter |
| 88 | Seq. ID No. 167 | Seq. ID No. 168 | Binding-protein-dependent transport s |
| 91b | Seq. ID No. 173 | Seq. ID No. 174 | NLPA lipoprotein |
| 99 | Seq. ID No. 191 | Seq. ID No. 192 | Phosphotransferase system, EIIC |
| 109d | Seq. ID No. 215 | Seq. ID No. 216 | ABC 3 transport family |
| 111b | Seq. ID No. 219 | Seq. ID No. 220 | ABC 3 transport family |
| 112a | Seq. ID No. 221 | Seq. ID No. 222 | ABC transporter |
| 112b | Seq. ID No. 223 | Seq. ID No. 224 | ABC transporter |
| 114 | Seq. ID No. 225 | Seq. ID No. 226 | Periplasmic solute binding protein fa |
| 121 | Seq. ID No. 233 | Seq. ID No. 234 | ABC transporter |
| 124c | Seq. ID No. 239 | Seq. ID No. 240 | Binding-protein-dependent transport s |
| 129b | Seq. ID No. 251 | Seq. ID No. 252 | Phosphotransferase system, EIIC |
| 129c | Seq. ID No. 253 | Seq. ID No. 254 | PTS system, Fructose specific IIB sub |
| 138a | Seq. ID No. 271 | Seq. ID No. 272 | E1-E2 ATPase |
| 145d | Seq. ID No. 291 | Seq. ID No. 292 | AbgT putative transporter family |
| 162c | Seq. ID No. 331 | Seq. ID No. 332 | Cobalt transport protein |
| 165a | Seq. ID No. 341 | Seq. ID No. 342 | ABC transporter |
| 167 | Seq. ID No. 345 | Seq. ID No. 346 | ABC transporter |
| 180b | Seq. ID No. 375 | Seq. ID No. 376 | eubacterial secY protein |
| 245c | Seq. ID No. 501 | Seq. ID No. 502 | Permease family |
| 246b | Seq. ID No. 505 | Seq. ID No. 506 | Cell cycle protein |
| 248b | Seq. ID No. 511 | Seq. ID No. 512 | Cell cycle protein |
| 255c | Seq. ID No. 529 | Seq. ID No. 530 | Sodium: solute symporter family |
| 273a | Seq. ID No. 567 | Seq. ID No. 568 | phosphotransferase system, EIIB |
| 273b | Seq. ID No. 569 | Seq. ID No. 570 | Phosphotransferase system, EIIC |
| 277c | Seq. ID No. 575 | Seq. ID No. 576 | ApbE family |
| 283b | Seq. ID No. 593 | Seq. ID No. 594 | Bacterial extracellular solute-bindin |
| 284a | Seq. ID No. 595 | Seq. ID No. 596 | Binding-protein-dependent transport s |
| 286a | Seq. ID No. 601 | Seq. ID No. 602 | ABC transporter |
| 311 | Seq. ID No. 637 | Seq. ID No. 638 | ABC transporter |
| 315 | Seq. ID No. 649 | Seq. ID No. 650 | ABC transporter |
| 318c | Seq. ID No. 661 | Seq. ID No. 662 | NLPA lipoprotein |
| 321 | Seq. ID No. 669 | Seq. ID No. 670 | NLPA lipoprotein |
| 324 | Seq. ID No. 675 | Seq. ID No. 676 | NLPA lipoprotein |
| 327c | Seq. ID No. 681 | Seq. ID No. 682 | Binding-protein-dependent transport s |
| 329 | Seq. ID No. 683 | Seq. ID No. 684 | ABC transporter |
| 342h | Seq. ID No. 729 | Seq. ID No. 730 | Uncharacterized membrane protein fami |
| 351 | Seq. ID No. 753 | Seq. ID No. 754 | ABC transporter |
| 358b | Seq. ID No. 779 | Seq. ID No. 780 | Na+/H+ antiporter family |
| 384b | Seq. ID No. 827 | Seq. ID No. 828 | Phosphotransferase system, EIIC |
| 391 | Seq. ID No. 839 | Seq. ID No. 840 | Glycosyl transferase family 8 |
| 418b | Seq. ID No. 895 | Seq. ID No. 896 | E1-E2 ATPase |
| 435 | Seq. ID No. 939 | Seq. ID No. 940 | Metallo-beta-lactamase superfamily |
| 450a | Seq. ID No. 969 | Seq. ID No. 970 | ABC transporter |
| 452e | Seq. ID No. 985 | Seq. ID No. 986 | Predicted permease |
| 464c | Seq. ID No. 1015 | Seq. ID No. 1016 | Cation transport protein |
| 472f | Seq. ID No. 1039 | Seq. ID No. 1040 | Cation transport protein |
| 492 | Seq. ID No. 1075 | Seq. ID No. 1076 | NLPA lipoprotein |
| 524b | Seq. ID No. 1137 | Seq. ID No. 1138 | 60 Kd inner membrane protein |
| 533 | Seq. ID No. 1155 | Seq. ID No. 1156 | Phosphotransferase system, EIIC |
| 539c | Seq. ID No. 1163 | Seq. ID No. 1164 | ABC transporter |
| 543a | Seq. ID No. 1175 | Seq. ID No. 1176 | ABC transporter |
| 561 | Seq. ID No. 1223 | Seq. ID No. 1224 | ABC transporter |
| 564c | Seq. ID No. 1231 | Seq. ID No. 1232 | BioY family |
| 570b | Seq. ID No. 1237 | Seq. ID No. 1238 | ABC transporter |
| 573 | Seq. ID No. 1241 | Seq. ID No. 1242 | ABC transporter |
| 586a | Seq. ID No. 1261 | Seq. ID No. 1262 | Large-conductance mechanosensitive ch |
| 613d | Seq. ID No. 1331 | Seq. ID No. 1332 | Sodium/hydrogen exchanger family |
| 618 | Seq. ID No. 1339 | Seq. ID No. 1340 | Mechanosensitive ion channel |
| 631d | Seq. ID No. 1369 | Seq. ID No. 1370 | 5′-nucleotidase, C-terminal domain |
| 698 | Seq. ID No. 1477 | Seq. ID No. 1478 | LrgA family |
| 722c | Seq. ID No. 1517 | Seq. ID No. 1518 | PTS system Galactitol-specific IIC co |
| 728b | Seq. ID No. 1531 | Seq. ID No. 1532 | PTS system sorbose-specific iic compo |
| 730d | Seq. ID No. 1539 | Seq. ID No. 1540 | PTS system mannose/fructose/sorbose f |
| 738c | Seq. ID No. 1549 | Seq. ID No. 1550 | PTS system Galactitol-specific IIC co |
| 744 | Seq. ID No. 1563 | Seq. ID No. 1564 | Bacterial extracellular solute-bindin |
| 745b | Seq. ID No. 1567 | Seq. ID No. 1568 | Binding-protein-dependent transport s |
| 746b | Seq. ID No. 1571 | Seq. ID No. 1572 | Binding-protein-dependent transport s |
| 747 | Seq. ID No. 1573 | Seq. ID No. 1574 | ABC transporter |
| 754d | Seq. ID No. 1589 | Seq. ID No. 1590 | PTS system, Lactose/Cellobiose specif |
| 758 | Seq. ID No. 1595 | Seq. ID No. 1596 | PTS system, Lactose/Cellobiose specif |
| 788b | Seq. ID No. 1647 | Seq. ID No. 1648 | Binding-protein-dependent transport s |
| 792 | Seq. ID No. 1657 | Seq. ID No. 1658 | ABC transporter |
| 859d | Seq. ID No. 1771 | Seq. ID No. 1772 | E1-E2 ATPase |
| 866b | Seq. ID No. 1785 | Seq. ID No. 1786 | ABC transporter |
| 868b | Seq. ID No. 1789 | Seq. ID No. 1790 | Binding-protein-dependent transport s |
| 885 | Seq. ID No. 1819 | Seq. ID No. 1820 | Histidine kinase-, DNA gyrase B-, and |
| 886 | Seq. ID No. 1821 | Seq. ID No. 1822 | D-alanyl-D-alanine carboxypeptidase |
| 977a | Seq. ID No. 1969 | Seq. ID No. 1970 | NLPA lipoprotein |
| 998b | Seq. ID No. 2015 | Seq. ID No. 2016 | PAP2 superfamily |
| 1016c | Seq. ID No. 2047 | Seq. ID No. 2048 | ABC transporter |
| 1036a | Seq. ID No. 2087 | Seq. ID No. 2088 | Binding-protein-dependent transport s |
| 1036b | Seq. ID No. 2089 | Seq. ID No. 2090 | Bacterial extracellular solute-bindin |
| 1037 | Seq. ID No. 2093 | Seq. ID No. 2094 | ABC transporter |
| 1047b | Seq. ID No. 2115 | Seq. ID No. 2116 | Periplasmic solute binding protein fa |
| 1049a | Seq. ID No. 2117 | Seq. ID No. 2118 | ABC transporter |
| 1050d | Seq. ID No. 2127 | Seq. ID No. 2128 | ABC 3 transport family |
| 1060b | Seq. ID No. 2141 | Seq. ID No. 2142 | Bacterial transferase hexapeptide (fo |
| 1077e | Seq. ID No. 2179 | Seq. ID No. 2180 | Polysaccharide biosynthesis protein |
| 1094b | Seq. ID No. 2207 | Seq. ID No. 2208 | Peptidase family M41 |
| 1100 | Seq. ID No. 2223 | Seq. ID No. 2224 | ABC transporter |
| 1127 | Seq. ID No. 2253 | Seq. ID No. 2254 | ABC transporter |
| 1140c | Seq. ID No. 2275 | Seq. ID No. 2276 | E1-E2 ATPase |
| 1149 | Seq. ID No. 2301 | Seq. ID No. 2302 | ABC transporter |
| 1152g | Seq. ID No. 2315 | Seq. ID No. 2316 | Binding-protein-dependent transport s |
| 1155g | Seq. ID No. 2329 | Seq. ID No. 2330 | Binding-protein-dependent transport s |
| 1165 | Seq. ID No. 2343 | Seq. ID No. 2344 | NLP/P60 family |
| 1167b | Seq. ID No. 2349 | Seq. ID No. 2350 | His Kinase A (phosphoacceptor) domain |
| 1171c | Seq. ID No. 2363 | Seq. ID No. 2364 | ABC transporter |
| 1179c | Seq. ID No. 2381 | Seq. ID No. 2382 | Metallo-beta-lactamase superfamily |
| 1180e | Seq. ID No. 2391 | Seq. ID No. 2392 | Phospholipase D. Active site motif |
| 1191 | Seq. ID No. 2417 | Seq. ID No. 2418 | ABC transporter |
| 1192b | Seq. ID No. 2421 | Seq. ID No. 2422 | Peptidase family M48 |
| 1194 | Seq. ID No. 2423 | Seq. ID No. 2424 | LemA family |
| 1197 | Seq. ID No. 2429 | Seq. ID No. 2430 | Peptidase family U7 |
| 1221a | Seq. ID No. 2481 | Seq. ID No. 2482 | ABC transporter |
| 1224a | Seq. ID No. 2487 | Seq. ID No. 2488 | CAAX amino terminal protease family |
| 1224b | Seq. ID No. 2489 | Seq. ID No. 2490 | CAAX amino terminal protease family |
| 1224c | Seq. ID No. 2491 | Seq. ID No. 2492 | CAAX amino terminal protease family |
| 1233a | Seq. ID No. 2509 | Seq. ID No. 2510 | ABC transporter |
| 1234c | Seq. ID No. 2519 | Seq. ID No. 2520 | Uncharacterised protein family (UPF00 |
| 1237 | Seq. ID No. 2525 | Seq. ID No. 2526 | LysM domain |
| 1245d | Seq. ID No. 2547 | Seq. ID No. 2548 | AbgT putative transporter family |
| 1246b | Seq. ID No. 2553 | Seq. ID No. 2554 | Metallo-beta-lactamase superfamily |
| 1250e | Seq. ID No. 2563 | Seq. ID No. 2564 | Sodium:dicarboxylate symporter family |
| 1258c | Seq. ID No. 2587 | Seq. ID No. 2588 | E1-E2 ATPase |
| 1266b | Seq. ID No. 2599 | Seq. ID No. 2600 | AzIC protein |
| 1268b | Seq. ID No. 2607 | Seq. ID No. 2608 | ABC transporter |
| 1271 | Seq. ID No. 2613 | Seq. ID No. 2614 | ABC transporter |
| 1288 | Seq. ID No. 2651 | Seq. ID No. 2652 | Cell envelope-related transcriptional |
| 1305b | Seq. ID No. 2675 | Seq. ID No. 2676 | ABC transporter |
| 1308b | Seq. ID No. 2681 | Seq. ID No. 2682 | Predicted permease |
| 1308e | Seq. ID No. 2687 | Seq. ID No. 2688 | Predicted permease |
| 1315 | Seq. ID No. 2693 | Seq. ID No. 2694 | Histidine kinase-, DNA gyrase B-, and |
| 1318 | Seq. ID No. 2699 | Seq. ID No. 2700 | Phosphate-binding protein |
| 1319b | Seq. ID No. 2703 | Seq. ID No. 2704 | Binding-protein-dependent transport s |
| 1320c | Seq. ID No. 2709 | Seq. ID No. 2710 | Binding-protein-dependent transport s |
| 1321 | Seq. ID No. 2711 | Seq. ID No. 2712 | ABC transporter |
| 1333 | Seq. ID No. 2727 | Seq. ID No. 2728 | ABC transporter |
| 1348 | Seq. ID No. 2753 | Seq. ID No. 2754 | Prolipoprotein diacylglyceryl transfe |
| 1384a | Seq. ID No. 2819 | Seq. ID No. 2820 | Preprotein translocase SecG subunit |
| 1384b | Seq. ID No. 2821 | Seq. ID No. 2822 | Preprotein translocase SecG subunit |
| 1384c | Seq. ID No. 2823 | Seq. ID No. 2824 | Preprotein translocase SecG subunit |
| 1408 | Seq. ID No. 2867 | Seq. ID No. 2868 | ABC transporter |
| 1410 | Seq. ID No. 2871 | Seq. ID No. 2872 | ABC transporter |
| 1426b | Seq. ID No. 2907 | Seq. ID No. 2908 | Ribonuclease BN-like family |
| 1431 | Seq. ID No. 2919 | Seq. ID No. 2920 | ABC transporter |
| 1442b | Seq. ID No. 2957 | Seq. ID No. 2958 | ABC transporter |
| 1447b | Seq. ID No. 2971 | Seq. ID No. 2972 | Sodium/glutamate symporter |
| 1451 | Seq. ID No. 2979 | Seq. ID No. 2980 | ABC transporter |
| 1457c | Seq. ID No. 2999 | Seq. ID No. 3000 | Na+/Pi-cotransporter |
| 1462b | Seq. ID No. 3005 | Seq. ID No. 3006 | ABC transporter |
| 1466d | Seq. ID No. 3019 | Seq. ID No. 3020 | ABC transporter |
| 1471 | Seq. ID No. 3035 | Seq. ID No. 3036 | Ion transport protein |
| 1484b | Seq. ID No. 3065 | Seq. ID No. 3066 | Divalent cation transporter |
| 1488g | Seq. ID No. 3085 | Seq. ID No. 3086 | Divalent cation transporter |
| 1517c | Seq. ID No. 3149 | Seq. ID No. 3150 | Mechanosensitive ion channel |
| 1530b | Seq. ID No. 3165 | Seq. ID No. 3166 | Transglycosylase |
| 1543 | Seq. ID No. 3183 | Seq. ID No. 3184 | NLP/P60 family |
| 1554c | Seq. ID No. 3203 | Seq. ID No. 3204 | Bacterial type II secretion system pr |
| 1556c | Seq. ID No. 3211 | Seq. ID No. 3212 | ABC transporter |
| 1573 | Seq. ID No. 3251 | Seq. ID No. 3252 | ABC transporter |
| 1578 | Seq. ID No. 3259 | Seq. ID No. 3260 | ABC transporter |
| 1606b | Seq. ID No. 3319 | Seq. ID No. 3320 | Histidine kinase-, DNA gyrase B-, and |
| 1607b | Seq. ID No. 3323 | Seq. ID No. 3324 | 60 Kd inner membrane protein |
| 1616b | Seq. ID No. 3335 | Seq. ID No. 3336 | FtsK/SpoIIIE family |
| 1619 | Seq. ID No. 3337 | Seq. ID No. 3338 | Basic membrane protein |
| 1623c | Seq. ID No. 3347 | Seq. ID No. 3348 | ABC transporter |
| 1626b | Seq. ID No. 3359 | Seq. ID No. 3360 | Branched-chain amino acid transport s |
| 1634c | Seq. ID No. 3377 | Seq. ID No. 3378 | Mannosyl-glycoprotein endo-beta-N-ace |
| 1639b | Seq. ID No. 3379 | Seq. ID No. 3380 | LysM domain |
| 1646f | Seq. ID No. 3399 | Seq. ID No. 3400 | Polysaccharide biosynthesis protein |
| 1659c | Seq. ID No. 3425 | Seq. ID No. 3426 | Na+/H+ antiporter family |
| 1683b | Seq. ID No. 3483 | Seq. ID No. 3484 | Ribonuclease BN-like family |
| 1688c | Seq. ID No. 3495 | Seq. ID No. 3496 | Glycosyl transferase |
| 1689a | Seq. ID No. 3497 | Seq. ID No. 3498 | Glycosyl transferase |
| 1694d | Seq. ID No. 3519 | Seq. ID No. 3520 | Cell envelope-related transcriptional |
| 1699b | Seq. ID No. 3529 | Seq. ID No. 3530 | Polysaccharide biosynthesis protein |
| 1737b | Seq. ID No. 3599 | Seq. ID No. 3600 | GA module |
| 1743 | Seq. ID No. 3611 | Seq. ID No. 3612 | GA module |
| 1744b | Seq. ID No. 3615 | Seq. ID No. 3616 | GA module |
| 1747 | Seq. ID No. 3619 | Seq. ID No. 3620 | GA module |
| 1769b | Seq. ID No. 3663 | Seq. ID No. 3664 | Type IV leader peptidase family |
| 1804b | Seq. ID No. 3739 | Seq. ID No. 3740 | Na+/H+ antiporter subunit |
| 1808d | Seq. ID No. 3759 | Seq. ID No. 3760 | NADH-Ubiquinone/plastoquinone (comple |
| 1809f | Seq. ID No. 3771 | Seq. ID No. 3772 | NADH-Ubiquinone/plastoquinone (comple |
| 1816c | Seq. ID No. 3797 | Seq. ID No. 3798 | Transglycosylase |
| 1833 | Seq. ID No. 3825 | Seq. ID No. 3826 | 5′-nucleotidase, C-terminal domain |
| 1838c | Seq. ID No. 3841 | Seq. ID No. 3842 | ABC transporter |
| 1842d | Seq. ID No. 3855 | Seq. ID No. 3856 | Phosphotransferase system, EIIC |
| 1845c | Seq. ID No. 3863 | Seq. ID No. 3864 | Voltage gated chloride channel |
| 1861b | Seq. ID No. 3901 | Seq. ID No. 3902 | ABC transporter |
| 1875c | Seq. ID No. 3929 | Seq. ID No. 3930 | Signal peptidase I |
| 1925 | Seq. ID No. 4015 | Seq. ID No. 4016 | Anion-transporting ATPase |
| 1926 | Seq. ID No. 4017 | Seq. ID No. 4018 | ABC transporter |
| 1939c | Seq. ID No. 4055 | Seq. ID No. 4056 | ABC transporter |
| 1946c | Seq. ID No. 4075 | Seq. ID No. 4076 | Sodium: alanine symporter family |
| 1951c | Seq. ID No. 4085 | Seq. ID No. 4086 | ABC transporter |
| 1953 | Seq. ID No. 4089 | Seq. ID No. 4090 | Patatin-like phospholipase |
| 1957 | Seq. ID No. 4099 | Seq. ID No. 4100 | ABC transporter |
| 1958c | Seq. ID No. 4105 | Seq. ID No. 4106 | Branched-chain amino acid transport s |
| 1969 | Seq. ID No. 4129 | Seq. ID No. 4130 | Signal peptidase (SPase) II |
| 2008 | Seq. ID No. 4203 | Seq. ID No. 4204 | Prokaryotic diacylglycerol kinase |
| 2023b | Seq. ID No. 4243 | Seq. ID No. 4244 | Rhomboid family |
| 2024d | Seq. ID No. 4251 | Seq. ID No. 4252 | Penicillin binding protein transpepti |
| 2074b | Seq. ID No. 4343 | Seq. ID No. 4344 | N-acetylmuramoyl-L-alanine amidase |
| 2094 | Seq. ID No. 4383 | Seq. ID No. 4384 | ABC transporter |
| 2142b | Seq. ID No. 4475 | Seq. ID No. 4476 | Phosphatidate cytidylyltransferase |
| 2146 | Seq. ID No. 4481 | Seq. ID No. 4482 | NLPA lipoprotein |
| 2147c | Seq. ID No. 4487 | Seq. ID No. 4488 | Binding-protein-dependent transport s |
| 2149b | Seq. ID No. 4491 | Seq. ID No. 4492 | ABC transporter |
| 2165 | Seq. ID No. 4533 | Seq. ID No. 4534 | Uncharacterised protein family (UPF01 |
| 2212b | Seq. ID No. 4627 | Seq. ID No. 4628 | LysM domain |
| 2249 | Seq. ID No. 4705 | Seq. ID No. 4706 | Bacterial extracellular solute-bindin |
| 2253d | Seq. ID No. 4713 | Seq. ID No. 4714 | Binding-protein-dependent transport s |
| 2255b | Seq. ID No. 4717 | Seq. ID No. 4718 | Binding-protein-dependent transport s |
| 2256 | Seq. ID No. 4719 | Seq. ID No. 4720 | ABC transporter |
| 2260c | Seq. ID No. 4729 | Seq. ID No. 4730 | ABC transporter |
| 2288 | Seq. ID No. 4789 | Seq. ID No. 4790 | Patatin-like phospholipase |
| 2290 | Seq. ID No. 4791 | Seq. ID No. 4792 | ATP synthase subunit D |
| 2291a | Seq. ID No. 4793 | Seq. ID No. 4794 | ATP synthase alpha/beta family, nucle |
| 2292c | Seq. ID No. 4803 | Seq. ID No. 4804 | ATP synthase alpha/beta family, nucle |
| 2300 | Seq. ID No. 4813 | Seq. ID No. 4814 | ATP synthase subunit C |
| 2301 | Seq. ID No. 4815 | Seq. ID No. 4816 | V-type ATPase 116 kDa subunit family |
| 2307 | Seq. ID No. 4825 | Seq. ID No. 4826 | Binding-protein-dependent transport s |
| 2309c | Seq. ID No. 4833 | Seq. ID No. 4834 | Binding-protein-dependent transport s |
| 2311 | Seq. ID No. 4837 | Seq. ID No. 4838 | ABC transporter |
| 2329d | Seq. ID No. 4875 | Seq. ID No. 4876 | Competence protein |
| 2331 | Seq. ID No. 4879 | Seq. ID No. 4880 | Helix-hairpin-helix motif |
| 2357 | Seq. ID No. 4919 | Seq. ID No. 4920 | Histidine kinase-, DNA gyrase B-, and |
| 2363b | Seq. ID No. 4931 | Seq. ID No. 4932 | Uncharacterized BCR, YceG family COG1 |
| 2370 | Seq. ID No. 4947 | Seq. ID No. 4948 | Bacterial transferase hexapeptide (fo |
| 2387 | Seq. ID No. 4985 | Seq. ID No. 4986 | Uncharacterized BCR, YitT family COG1 |
| 2428c | Seq. ID No. 5077 | Seq. ID No. 5078 | Pyridoxal-phosphate dependent enzyme |
| 2453 | Seq. ID No. 5115 | Seq. ID No. 5116 | Periplasmic binding protein |
| 2455 | Seq. ID No. 5117 | Seq. ID No. 5118 | ABC transporter |
| 2456e | Seq. ID No. 5127 | Seq. ID No. 5128 | FecCD transport family |
| 2458g | Seq. ID No. 5141 | Seq. ID No. 5142 | FecCD transport family |
| 2482 | Seq. ID No. 5193 | Seq. ID No. 5194 | YGGT family |
| 2495b | Seq. ID No. 5215 | Seq. ID No. 5216 | Glycosyl transferase |
| 2497b | Seq. ID No. 5219 | Seq. ID No. 5220 | Penicillin binding protein transpepti |
| 2520b | Seq. ID No. 5275 | Seq. ID No. 5276 | PEP-utilizing enzyme, TIM barrel doma |
| 2564d | Seq. ID No. 5381 | Seq. ID No. 5382 | D-alanyl-D-alanine carboxypeptidase |
| 2568 | Seq. ID No. 5389 | Seq. ID No. 5390 | ABC transporter |
| 2582c | Seq. ID No. 5409 | Seq. ID No. 5410 | Cobalt transport protein |
| 2585 | Seq. ID No. 5411 | Seq. ID No. 5412 | ABC transporter |
| 2617a | Seq. ID No. 5475 | Seq. ID No. 5476 | ABC transporter |
| 2623b | Seq. ID No. 5491 | Seq. ID No. 5492 | PPIC-type PPIASE domain |
| 2629b | Seq. ID No. 5513 | Seq. ID No. 5514 | Bacterial extracellular solute-bindin |
| 2633a | Seq. ID No. 5519 | Seq. ID No. 5520 | Bacterial extracellular solute-bindin |
| 2633d | Seq. ID No. 5525 | Seq. ID No. 5526 | Bacterial extracellular solute-bindin |
| 2637a | Seq. ID No. 5529 | Seq. ID No. 5530 | Bacterial extracellular solute-bindin |
| 2643b | Seq. ID No. 5537 | Seq. ID No. 5538 | Transglycosylase |
| 2655b | Seq. ID No. 5557 | Seq. ID No. 5558 | Uncharacterized secreted proteins, Ya |
| 2666b | Seq. ID No. 5581 | Seq. ID No. 5582 | MutS family, N-terminal putative DNA |
| 2674b | Seq. ID No. 5599 | Seq. ID No. 5600 | Carbon starvation protein CstA |
| 2688c | Seq. ID No. 5637 | Seq. ID No. 5638 | CDP-alcohol phosphatidyltransferase |
| 2718c | Seq. ID No. 5685 | Seq. ID No. 5686 | Uncharacterized BCR, COG1963 |
| 2734c | Seq. ID No. 5723 | Seq. ID No. 5724 | Cation efflux family |
| 2747b | Seq. ID No. 5755 | Seq. ID No. 5756 | Metallo-beta-lactamase superfamily |
| 2748c | Seq. ID No. 5761 | Seq. ID No. 5762 | Uncharacterized membrane protein fami |
| 2801b | Seq. ID No. 5895 | Seq. ID No. 5896 | HAMP domain |
| 2805b | Seq. ID No. 5901 | Seq. ID No. 5902 | Predicted permease |
| 2809 | Seq. ID No. 5903 | Seq. ID No. 5904 | ABC transporter |
| 2810d | Seq. ID No. 5911 | Seq. ID No. 5912 | Predicted permease |
| 2812 | Seq. ID No. 5913 | Seq. ID No. 5914 | ABC transporter |
| 2816 | Seq. ID No. 5919 | Seq. ID No. 5920 | ABC transporter |
| 2818b | Seq. ID No. 5923 | Seq. ID No. 5924 | Binding-protein-dependent transport s |
| 2818e | Seq. ID No. 5929 | Seq. ID No. 5930 | Bacterial extracellular solute-bindin |
| 2856c | Seq. ID No. 6001 | Seq. ID No. 6002 | E1-E2 ATPase |
| 2881 | Seq. ID No. 6043 | Seq. ID No. 6044 | Mannosyl-glycoprotein endo-beta-N-ace |
| 2883d | Seq. ID No. 6053 | Seq. ID No. 6054 | Predicted permease |
| 2886a | Seq. ID No. 6055 | Seq. ID No. 6056 | ABC transporter |
| 2900b | Seq. ID No. 6089 | Seq. ID No. 6090 | LysM domain |
| 2902 | Seq. ID No. 6095 | Seq. ID No. 6096 | LysM domain |
| 2909d | Seq. ID No. 6111 | Seq. ID No. 6112 | Sulfatase |
| 2913b | Seq. ID No. 6117 | Seq. ID No. 6118 | Histidine kinase-, DNA gyrase B-, and |
| 2931c | Seq. ID No. 6165 | Seq. ID No. 6166 | Sodium: solute symporter family |
| 2933b | Seq. ID No. 6169 | Seq. ID No. 6170 | BNR/Asp-box repeat |
| 2941c | Seq. ID No. 6187 | Seq. ID No. 6188 | ABC transporter |
| 2943d | Seq. ID No. 6195 | Seq. ID No. 6196 | ABC transporter |
| 2954 | Seq. ID No. 6219 | Seq. ID No. 6220 | Bacitracin resistance protein BacA |
| 2955g | Seq. ID No. 6233 | Seq. ID No. 6234 | Permease family |
| 2971a | Seq. ID No. 6269 | Seq. ID No. 6270 | ABC transporter |
| 2974b | Seq. ID No. 6275 | Seq. ID No. 6276 | Predicted permease |
| 2977a | Seq. ID No. 6279 | Seq. ID No. 6280 | Chromate transporter |
| 2979 | Seq. ID No. 6287 | Seq. ID No. 6288 | Chromate transporter |
| 2980e | Seq. ID No. 6297 | Seq. ID No. 6298 | NQR2, RnfD, RnfE family |
| 2984 | Seq. ID No. 6299 | Seq. ID No. 6300 | Respiratory-chain NADH dehydrogenase |
| 2986c | Seq. ID No. 6305 | Seq. ID No. 6306 | NQR2, RnfD, RnfE family |
| 2988c | Seq. ID No. 6311 | Seq. ID No. 6312 | Respiratory-chain NADH dehydrogenase |
| 3017 | Seq. ID No. 6369 | Seq. ID No. 6370 | ABC transporter |
| 3022 | Seq. ID No. 6379 | Seq. ID No. 6380 | Clp amino terminal domain |
| 3116 | Seq. ID No. 6587 | Seq. ID No. 6588 | Peptidase C1-like family |
| 3118b | Seq. ID No. 6591 | Seq. ID No. 6592 | Peptidase C1-like family |

Listed in Table 8 are ORFs whose BlastP results predict for possible secreted, surface or membrane localization. Keywords used in the search are: MHC, murein, mycolic, NisP, penicillin, peptidoglycan, periplasmic, phosphatidate, pilus, protease, receptor, response regulator, sacculus, secreted protein, sensor protein, signal, surface, teichoic, toxin, transferring, transmembrane, virulence, vitronectin, wall, spor, sporulatio, immunoglobulin, IgG, IgE, IgM, IgD, cueat, teichoic, serum, transport, transporter, channel, porin, hemolysin, phosphatase, vaccine, polysaccharide, rhamnose, dexB, aliA, cap, cps, wze, wzh. A total of 319 ORFs whose BlastP results predict for a possible secreted, surface or membrane localization for the polypeptides encoded by the ORFs are listed in Table 8.

TABLE 8
_BLASTP

| | DNA | Protein | | | |
|---|---|---|---|---|---|
| ORF | SEQ ID | SEQ ID | | | Blast ‘e’ |
| Number | Number | Number | Keyword | Description | Value |
| 4b | Seq. ID No. | Seq. ID No. | transport | transporter, Bacillus | 4.00E−15 |
| | 9 | 10 | | halodurans Bacteria; | |
| | | | | Firmicu . . . | |
| 10 | Seq. ID No. | Seq. ID No. | virulence | Neisseria | 5.00E−35 |
| | 17 | 18 | | meningitidis | |
| | | | | virulence pro . . . | |
| | | | | Aau72997 . . . | |
| 38a | Seq. ID No. | Seq. ID No. | porin | Cephalosporin C | 6.00E−30 |
| | 45 | 46 | | amidase enzyme. | |
| | | | | 3/1991 Aar10055 . . . | |
| 39a | Seq. ID No. | Seq. ID No. | capsular | PgsC, Bacillus | 1.00E−18 |
| | 49 | 50 | | subtilis Bacteria; | |
| | | | | Firmicutes; Baci . . . | |
| 40 | Seq. ID No. | Seq. ID No. | capB | CapB, Bacillus | 9.00E−95 |
| | 53 | 54 | | subtilis Bacteria; | |
| | | | | Firmicutes; Baci . . . | |
| 41c | Seq. ID No. | Seq. ID No. | capA | PgsA, Bacillus | 2.00E−44 |
| | 59 | 60 | | subtilis Bacteria; | |
| | | | | Firmicutes; Baci . . . | |
| 51c | Seq. ID No. | Seq. ID No. | membrane | putative | 2.00E−44 |
| | 89 | 90 | | transmembrane | |
| | | | | symporter, | |
| | | | | Campylobacter je . . . | |
| 80c | Seq. ID No. | Seq. ID No. | transport | Listeria | 6.00E−32 |
| | 145 | 146 | | monocytogenes | |
| | | | | Bacteria; Firmicutes; | |
| | | | | Bacil . . . | |
| 85c | Seq. ID No. | Seq. ID No. | envelop | H. pylori cell | 2.00E−50 |
| | 157 | 158 | | envelope transporter | |
| | | | | . . . Aaw20861 . . . | |
| 88 | Seq. ID No. | Seq. ID No. | TRANSMEMBRANE | PROBABLE | 2.00E−24 |
| | 167 | 168 | | TRANSMEMBRANE | |
| | | | | ABC | |
| | | | | TRANSPORTER | |
| | | | | PROTEIN, Ra . . . | |
| 91a | Seq. ID No. | Seq. ID No. | membrane | outer membrane | 5.00E−25 |
| | 171 | 172 | | protein, Xylella | |
| | | | | fastidiosa 9a5c Ba . . . | |
| 99 | Seq. ID No. | Seq. ID No. | membrane | putative PTS | 8.00E−38 |
| | 191 | 192 | | transmembrane | |
| | | | | compon . . . | |
| | | | | AL096852 . . . | |
| 109d | Seq. ID No. | Seq. ID No. | membrane | High-affinity zinc | 2.00E−60 |
| | 215 | 216 | | uptake system | |
| | | | | membran, | |
| | | | | Fusobact . . . | |
| 111a | Seq. ID No. | Seq. ID No. | transporter | YtgC, Bacillus | 7.00E−34 |
| | 217 | 218 | | subtilis Bacteria; | |
| | | | | Firmicutes; Baci . . . | |
| 114 | Seq. ID No. | Seq. ID No. | adhesin | surface adhesin A | 6.00E−42 |
| | 225 | 226 | | precursor, Bacillus | |
| | | | | halodurans B . . . | |
| 124c | Seq. ID No. | Seq. ID No. | membrane | ABC transporter | 1.00E−105 |
| | 239 | 240 | | membrane-spanning | |
| | | | | permease, Strep . . . | |
| 129c | Seq. ID No. | Seq. ID No. | immunogenic | Propionibacterium | 5.00E−77 |
| | 253 | 254 | | acnes immunogenic | |
| | | | | . . . Aau44300 . . . | |
| 138b | Seq. ID No. | Seq. ID No. | transport | cadmium- | 1.00E−114 |
| | 273 | 274 | | transporting | |
| | | | | ATPase, Bacillus | |
| | | | | halodurans B . . . | |
| 143 | Seq. ID No. | Seq. ID No. | immunogenic | Propionibacterium | 1.00E−14 |
| | 277 | 278 | | acnes immunogenic | |
| | | | | . . . Aau4245 . . . | |
| 145c | Seq. ID No. | Seq. ID No. | membrane | hypothetical integral | 1.00E−92 |
| | 289 | 290 | | membrane protein, | |
| | | | | Neisseria . . . | |
| 162b | Seq. ID No. | Seq. ID No. | membrane | putative ABC | 2.00E−58 |
| | 329 | 330 | | transporter | |
| | | | | membrane-spannin, | |
| | | | | Strepto . . . | |
| 165b | Seq. ID No. | Seq. ID No. | transport | ABC transporter | 7.00E−90 |
| | 343 | 344 | | ATP-binding protein, | |
| | | | | Lactococcus l . . . | |
| 167 | Seq. ID No. | Seq. ID No. | transport | putative ABC | 5.00E−99 |
| | 345 | 346 | | transporter, | |
| | | | | Enterococcus | |
| | | | | faecium Bac . . . | |
| 180b | Seq. ID No. | Seq. ID No. | membrane | preprotein | 1.00E−101 |
| | 375 | 376 | | translocase, SecY | |
| | | | | subunit, | |
| | | | | Streptococcu . . . | |
| 245c | Seq. ID No. | Seq. ID No. | transporter | uracil permease, | 2.00E−80 |
| | 501 | 502 | | Bacillus caldolyticus | |
| | | | | Bacteria; F . . . | |
| 246b | Seq. ID No. | Seq. ID No. | sporulation | stage V sporulation | 3.00E−46 |
| | 505 | 506 | | protein E, Bacillus | |
| | | | | halodurans . . . | |
| 248b | Seq. ID No. | Seq. ID No. | sporulation | stage V sporulation | 3.00E−46 |
| | 511 | 512 | | protein E, Bacillus | |
| | | | | halodurans . . . | |
| 249b | Seq. ID No. | Seq. ID No. | peptidoglycan | putative | 4.00E−47 |
| | 515 | 516 | | peptidoglycan | |
| | | | | GlcNAc deacetylase, | |
| | | | | Strepto . . . | |
| 255c | Seq. ID No. | Seq. ID No. | transporter | putative | 1.00E−52 |
| | 529 | 530 | | sodium/glucose | |
| | | | | cotransporter, | |
| | | | | Salmonella . . . | |
| 273b | Seq. ID No. | Seq. ID No. | immunogenic | Propionibacterium | 5.00E−56 |
| | 569 | 570 | | acnes immunogenic | |
| | | | | . . . Aau41330 . . . | |
| 277c | Seq. ID No. | Seq. ID No. | lipoprotein | Listeria | 4.00E−91 |
| | 575 | 576 | | monocytogenes | |
| | | | | Bacteria; Firmicutes; | |
| | | | | Bacil . . . | |
| 283b | Seq. ID No. | Seq. ID No. | transport | probable amino acid | 3.00E−19 |
| | 593 | 594 | | ABC transporter, | |
| | | | | Clostridium p . . . | |
| 284a | Seq. ID No. | Seq. ID No. | membrane | membrane protein, | 5.00E−39 |
| | 595 | 596 | | Vibrio harveyi | |
| | | | | Bacteria; Proteob . . . | |
| 311 | Seq. ID No. | Seq. ID No. | transporter | ABC transporter | 5.00E−58 |
| | 637 | 638 | | (ATP-binding | |
| | | | | protein), Bacillus | |
| | | | | ha . . . | |
| 318b | Seq. ID No. | Seq. ID No. | lipoprotein | PUTATIVE OUTER | 4.00E−34 |
| | 659 | 660 | | MEMBRANE | |
| | | | | LIPOPROTEIN, | |
| | | | | Sinorhizobium . . . | |
| 321 | Seq. ID No. | Seq. ID No. | lipoprotein | Staphylococcus | 1.00E−54 |
| | 669 | 670 | | aureus subsp. | |
| | | | | aureus MW2 | |
| | | | | Bacteria; . . . | |
| 324 | Seq. ID No. | Seq. ID No. | lipoprotein | Staphylococcus | 1.00E−55 |
| | 675 | 676 | | aureus subsp. | |
| | | | | aureus MW2 | |
| | | | | Bacteria; . . . | |
| 327c | Seq. ID No. | Seq. ID No. | transport | ABC transporter, | 9.00E−36 |
| | 681 | 682 | | permease protein, | |
| | | | | putative, Strep . . . | |
| 342h | Seq. ID No. | Seq. ID No. | transmembrane | putative | 1.00E−33 |
| | 729 | 730 | | transmembrane | |
| | | | | efflux protein, | |
| | | | | Methanosarc . . . | |
| 358b | Seq. ID No. | Seq. ID No. | transport | transport protein, | 1.00E−119 |
| | 779 | 780 | | Fusobacterium | |
| | | | | nucleatum subsp. . . . | |
| 382b | Seq. ID No. | Seq. ID No. | secreted protein | putative secreted | 2.00E−37 |
| | 819 | 820 | | protein, Listeria | |
| | | | | innocua Bacter . . . | |
| 418b | Seq. ID No. | Seq. ID No. | transport | metal transporting | 1.00E−138 |
| | 895 | 896 | | ATPase, | |
| | | | | Lactococcus lactis | |
| | | | | subs . . . | |
| 427a | Seq. ID No. | Seq. ID No. | capsular | unknown, | 1.00E−46 |
| | 921 | 922 | | Staphylococcus | |
| | | | | aureus Bacteria; | |
| | | | | Firmicute . . . | |
| 431 | Seq. ID No. | Seq. ID No. | protease | Listeria innocua | 2.00E−41 |
| | 935 | 936 | | Bacteria; Firmicutes; | |
| | | | | Bacillus/Cl . . . | |
| 441b | Seq. ID No. | Seq. ID No. | response regulator | histidine kinase | 1.00E−135 |
| | 955 | 956 | | YycG, | |
| | | | | Staphylococcus | |
| | | | | aureus Bacte . . . | |
| 448 | Seq. ID No. | Seq. ID No. | transporter | accessory protein, | 1.00E−17 |
| | 967 | 968 | | Enterococcus | |
| | | | | faecalis subsp. li . . . | |
| 452e | Seq. ID No. | Seq. ID No. | transporter | membrane subunit, | 8.00E−55 |
| | 985 | 986 | | Enterococcus | |
| | | | | faecalis subsp. liq . . . | |
| 464d | Seq. ID No. | Seq. ID No. | membrane | Trk transporter | 4.00E−82 |
| | 1017 | 1018 | | membrane-spanning | |
| | | | | protein - K, Str . . . | |
| 472f | Seq. ID No. | Seq. ID No. | membrane | potassium uptake | 1.00E−73 |
| | 1039 | 1040 | | protein, Trk family, | |
| | | | | Streptococcu . . . | |
| 492 | Seq. ID No. | Seq. ID No. | lipoprotein | putative outer | 4.00E−28 |
| | 1075 | 1076 | | membrane | |
| | | | | lipoprotein, | |
| | | | | Salmonella ty . . . | |
| 499a | Seq. ID No. | Seq. ID No. | phosphatase | Exopolyphosphatase- | 1.00E−110 |
| | 1089 | 1090 | | related proteins, | |
| | | | | Thermoanaerob . . . | |
| 524b | Seq. ID No. | Seq. ID No. | sporulation | stage III sporulation, | 4.00E−38 |
| | 1137 | 1138 | | Bacillus subtilis | |
| | | | | Bacteria; . . . | |
| 561 | Seq. ID No. | Seq. ID No. | membrane | ABC transporter | 2.00E−63 |
| | 1223 | 1224 | | ATP- | |
| | | | | binding/membrane | |
| | | | | spannin, Stre . . . | |
| 573 | Seq. ID No. | Seq. ID No. | transport | ABC-type | 2.00E−61 |
| | 1241 | 1242 | | multidrug/protein/lipid | |
| | | | | transpor, | |
| | | | | Thermoa . . . | |
| 609a | Seq. ID No. | Seq. ID No. | spore | spore wall protein 2 | 6.00E−23 |
| | 1315 | 1316 | | precursor, | |
| | | | | Encephalitozoon | |
| | | | | in . . . | |
| 613d | Seq. ID No. | Seq. ID No. | TRANSMEMBRANE | HYPOTHETICAL | 5.00E−13 |
| | 1331 | 1332 | | TRANSMEMBRANE | |
| | | | | PROTEIN, | |
| | | | | Ralstonia sola . . . | |
| 631d | Seq. ID No. | Seq. ID No. | Signal | PROBABLE 5′- | 4.00E−60 |
| | 1369 | 1370 | | NUCLEOTIDASE | |
| | | | | PRECURSOR | |
| | | | | (SIGNAL, Sinorhi . . . | |
| 687 | Seq. ID No. | Seq. ID No. | transmembrane | Chlamydia | 6.00E−45 |
| | 1459 | 1460 | | pneumoniae | |
| | | | | transmembrane p . . . | |
| | | | | Aay34923 . . . | |
| 722c | Seq. ID No. | Seq. ID No. | immunogenic | Propionibacterium | 3.00E−16 |
| | 1517 | 1518 | | acnes immunogenic | |
| | | | | . . . Aau6647 . . . | |
| 743b | Seq. ID No. | Seq. ID No. | transport | putative alpha- | 2.00E−27 |
| | 1561 | 1562 | | glucosidase, | |
| | | | | Alicyclobacillus | |
| | | | | acido . . . | |
| 744 | Seq. ID No. | Seq. ID No. | transport | Glicerol-3-phosphate | 1.00E−93 |
| | 1563 | 1564 | | ABC-transporter, | |
| | | | | Clostridium . . . | |
| 745b | Seq. ID No. | Seq. ID No. | transport | Glycerol-3- | 2.00E−49 |
| | 1567 | 1568 | | phosphate ABC- | |
| | | | | transporter, | |
| | | | | permeas, Clo . . . | |
| 746b | Seq. ID No. | Seq. ID No. | transport | glycerol-3-phosphate | 1.00E−51 |
| | 1571 | 1572 | | ABC transporter | |
| | | | | (permease), B . . . | |
| 747 | Seq. ID No. | Seq. ID No. | transport | Multiple sugar- | 2.00E−95 |
| | 1573 | 1574 | | binding ABC- | |
| | | | | transporter, MSM, | |
| | | | | Clost . . . | |
| 762b | Seq. ID No. | Seq. ID No. | cpsL | transposase, | 5.00E−44 |
| | 1601 | 1602 | | Streptococcus | |
| | | | | pneumoniae | |
| | | | | Bacteria; Fi . . . | |
| 763b | Seq. ID No. | Seq. ID No. | capsular | transposase, | 4.00E−16 |
| | 1607 | 1608 | | Staphylococcus | |
| | | | | aureus Bacteria; | |
| | | | | Firmi . . . | |
| 767 | Seq. ID No. | Seq. ID No. | capsular | transposase, | 4.00E−14 |
| | 1613 | 1614 | | Staphylococcus | |
| | | | | aureus Bacteria; | |
| | | | | Firmi . . . | |
| 770c | Seq. ID No. | Seq. ID No. | transport | acetylornitine | 2.00E−32 |
| | 1619 | 1620 | | deacetylase, Bacillus | |
| | | | | subtilis Bact . . . | |
| 785 | Seq. ID No. | Seq. ID No. | phosphatase | phosphatase, | 9.00E−29 |
| | 1641 | 1642 | | Lactococcus lactis | |
| | | | | subsp. lactis Bact . . . | |
| 788b | Seq. ID No. | Seq. ID No. | transport | Staphylococcus | 1.00E−19 |
| | 1647 | 1648 | | aureus subsp. | |
| | | | | aureus MW2 | |
| | | | | Bacteria; . . . | |
| 789c | Seq. ID No. | Seq. ID No. | transport | Staphylococcus | 3.00E−31 |
| | 1653 | 1654 | | aureus subsp. | |
| | | | | aureus MW2 | |
| | | | | Bacteria; . . . | |
| 792 | Seq. ID No. | Seq. ID No. | transport | Staphylococcus | 2.00E−87 |
| | 1657 | 1658 | | aureus subsp. | |
| | | | | aureus N315 | |
| | | | | Bacteria; . . . | |
| 794d | Seq. ID No. | Seq. ID No. | transporter | alkylphosphonate | 4.00E−26 |
| | 1665 | 1666 | | ABC tranporte, | |
| | | | | Bacillus haloduran . . . | |
| 813 | Seq. ID No. | Seq. ID No. | transporter | cadmium resistance | 3.00E−57 |
| | 1699 | 1700 | | transporter, putative, | |
| | | | | Streptoc . . . | |
| 830 | Seq. ID No. | Seq. ID No. | cpsA | Thermostable | 1.00E−50 |
| | 1713 | 1714 | | carboxypeptidase | |
| | | | | (cpsA-2), | |
| | | | | Sulfolobus . . . | |
| 851b | Seq. ID No. | Seq. ID No. | penicillin | B. lactofermentum | 3.00E−23 |
| | 1751 | 1752 | | penicillin binding . . . | |
| | | | | Aay33117 . . . | |
| 859d | Seq. ID No. | Seq. ID No. | transport | putative cation | 1.00E−146 |
| | 1771 | 1772 | | transporting P-ty . . . | |
| | | | | AF067954 . . . | |
| 866b | Seq. ID No. | Seq. ID No. | membrane | ATPase, Bacillus | 1.00E−124 |
| | 1785 | 1786 | | subtilis Bacteria; | |
| | | | | Firmicutes; GE . . . | |
| 868b | Seq. ID No. | Seq. ID No. | transporter | betaine ABC | 1.00E−71 |
| | 1789 | 1790 | | transporter | |
| | | | | permease and | |
| | | | | substrat, Lac . . . | |
| 870 | Seq. ID No. | Seq. ID No. | transporter | Pseudogene, similar | 1.00E−80 |
| | 1791 | 1792 | | to glycine-betaine | |
| | | | | bindin, Lis . . . | |
| 873c | Seq. ID No. | Seq. ID No. | transporter | Pseudogene, similar | 2.00E−73 |
| | 1797 | 1798 | | to glycine-betaine | |
| | | | | bindin, Lis . . . | |
| 878c | Seq. ID No. | Seq. ID No. | hemolysin | Alpha-hemolysin, | 1.00E−20 |
| | 1811 | 1812 | | Fusobacterium | |
| | | | | nucleatum subsp. | |
| | | | | nu . . . | |
| 880c | Seq. ID No. | Seq. ID No. | response regulator | putative response | 5.00E−76 |
| | 1817 | 1818 | | regulator, | |
| | | | | Lactobacillus sakei | |
| | | | | B . . . | |
| 885 | Seq. ID No. | Seq. ID No. | response regulator | putative histidine | 1.00E−82 |
| | 1819 | 1820 | | kinase, Lactobacillus | |
| | | | | sakei Bac . . . | |
| 886 | Seq. ID No. | Seq. ID No. | penicillin | D-alanine | 4.00E−55 |
| | 1821 | 1822 | | carboxypeptidase | |
| | | | | (PBP-5), Bacillus | |
| | | | | subti . . . | |
| 929b | Seq. ID No. | Seq. ID No. | membrane | PROBABLE | 1.00E−117 |
| | 1871 | 1872 | | BIFUNCTIONAL, | |
| | | | | Ralstonia | |
| | | | | solanacearum Bac . . . | |
| 939c | Seq. ID No. | Seq. ID No. | transporter | branch-chain amino | 3.00E−64 |
| | 1895 | 1896 | | acid transporter, | |
| | | | | Bacillus subt . . . | |
| 946 | Seq. ID No. | Seq. ID No. | phosphatase | deoxyuridine 5′- | 3.00E−30 |
| | 1905 | 1906 | | triphosphate | |
| | | | | nucleotidhydrolase, | |
| | | | | L . . . | |
| 949d | Seq. ID No. | Seq. ID No. | PROTEASE | PROBABLE ATP- | 1.00E−101 |
| | 1913 | 1914 | | DEPENDENT | |
| | | | | PROTEASE, . . . | |
| | | | | AL646064 . . . | |
| 951b | Seq. ID No. | Seq. ID No. | transporter | Pseudogene, similar | 4.00E−76 |
| | 1919 | 1920 | | to glycine-betaine | |
| | | | | bindin, Lis . . . | |
| 952b | Seq. ID No. | Seq. ID No. | Membrane | Membrane- | 3.00E−68 |
| | 1923 | 1924 | | associated protein | |
| | | | | containing a homolo, | |
| | | | | C . . . | |
| 972b | Seq. ID No. | Seq. ID No. | phosphatase | exopolyphosphatase, | 3.00E−60 |
| | 1965 | 1966 | | Bacillus halodurans | |
| | | | | Bacteria; . . . | |
| 977b | Seq. ID No. | Seq. ID No. | lipoprotein | PUTATIVE OUTER | 1.00E−50 |
| | 1971 | 1972 | | MEMBRANE | |
| | | | | LIPOPROTEIN, | |
| | | | | Sinorhizobium . . . | |
| 988d | Seq. ID No. | Seq. ID No. | membrane | putative conserved | 3.00E−35 |
| | 1999 | 2000 | | membrane protein, | |
| | | | | Listeria mono . . . | |
| 1016c | Seq. ID No. | Seq. ID No. | transporter | Na+ ABC transporter | 1.00E−41 |
| | 2047 | 2048 | | (ATP-binding | |
| | | | | protein), NATA, C . . . | |
| 1017d | Seq. ID No. | Seq. ID No. | transporter | Na+ ABC | 2.00E−17 |
| | 2055 | 2056 | | transporter, NATB, | |
| | | | | Clostridium | |
| | | | | acetobutyli . . . | |
| 1036c | Seq. ID No. | Seq. ID No. | transporter | Listeria | 1.00E−126 |
| | 2091 | 2092 | | monocytogenes | |
| | | | | Bacteria; Firmicutes; | |
| | | | | Bacil . . . | |
| 1037 | Seq. ID No. | Seq. ID No. | transporter | Glutamine ABC | 1.00E−93 |
| | 2093 | 2094 | | transporter (ATP- | |
| | | | | binding protein), C . . . | |
| 1047b | Seq. ID No. | Seq. ID No. | adhesion | YcdH, Bacillus | 3.00E−35 |
| | 2115 | 2116 | | subtilis Bacteria; | |
| | | | | Firmicutes; Baci . . . | |
| 1049b | Seq. ID No. | Seq. ID No. | transporter | Listeria innocua | 8.00E−92 |
| | 2119 | 2120 | | Bacteria; Firmicutes; | |
| | | | | Bacillus/Cl . . . | |
| 1050d | Seq. ID No. | Seq. ID No. | membrane | Listeria innocua | 4.00E−71 |
| | 2127 | 2128 | | Bacteria; Firmicutes; | |
| | | | | Bacillus/Cl . . . | |
| 1070b | Seq. ID No. | Seq. ID No. | sporulation | stage V sporulation | 3.00E−47 |
| | 2165 | 2166 | | protein C (peptidyl- | |
| | | | | tRN, Bacil . . . | |
| 1077e | Seq. ID No. | Seq. ID No. | membrane | Listeria innocua | 3.00E−53 |
| | 2179 | 2180 | | Bacteria; Firmicutes; | |
| | | | | Bacillus/Cl . . . | |
| 1094b | Seq. ID No. | Seq. ID No. | membrane | S. pneumoniae | 0 |
| | 2207 | 2208 | | putative ATPase | |
| | | | | involved in | |
| | | | | membrane . . . | |
| 1100 | Seq. ID No. | Seq. ID No. | transporter | probable ABC | 9.00E−27 |
| | 2223 | 2224 | | transporter, | |
| | | | | Clostridium | |
| | | | | perfringens . . . | |
| 1101b | Seq. ID No. | Seq. ID No. | transporter | ABC transporter | 2.00E−15 |
| | 2227 | 2228 | | ATP-binding protein, | |
| | | | | Xanthomonas a . . . | |
| 1111c | Seq. ID No. | Seq. ID No. | membrane | Listeria innocua | 2.00E−49 |
| | 2241 | 2242 | | Bacteria; Firmicutes; | |
| | | | | Bacillus/Cl . . . | |
| 1127 | Seq. ID No. | Seq. ID No. | transporter | ABC transporter | 7.00E−35 |
| | 2253 | 2254 | | ATP-binding protein, | |
| | | | | Clostridium a . . . | |
| 1135b | Seq. ID No. | Seq. ID No. | lipoprotein | putative lipoprotein, | 5.00E−29 |
| | 2263 | 2264 | | Streptomyces | |
| | | | | coelicolor A3(2 . . . | |
| 1138 | Seq. ID No. | Seq. ID No. | capsular | unknown, | 9.00E−60 |
| | 2269 | 2270 | | Staphylococcus | |
| | | | | aureus Bacteria; | |
| | | | | Firmicute . . . | |
| 1140c | Seq. ID No. | Seq. ID No. | transport | Listeria innocua | 2.00E−85 |
| | 2275 | 2276 | | Bacteria; Firmicutes; | |
| | | | | Bacillus/Cl . . . | |
| 1149 | Seq. ID No. | Seq. ID No. | transporter | putative ABC- | 1.00E−106 |
| | 2301 | 2302 | | transporter ATP- | |
| | | | | bind . . . AL627273 . . . | |
| 1152g | Seq. ID No. | Seq. ID No. | transporter | dipeptide ABC | 1.00E−49 |
| | 2315 | 2316 | | transporter, | |
| | | | | permease protei, | |
| | | | | Helico . . . | |
| 1155g | Seq. ID No. | Seq. ID No. | transporter | Staphylococcus | 3.00E−80 |
| | 2329 | 2330 | | aureus subsp. | |
| | | | | aureus MW2 | |
| | | | | Bacteria; . . . | |
| 1158b | Seq. ID No. | Seq. ID No. | lipoprotein | RGD-containing | 2.00E−94 |
| | 2333 | 2334 | | lipoprotein, | |
| | | | | Staphylococcus | |
| | | | | aureus . . . | |
| 1165 | Seq. ID No. | Seq. ID No. | peptidoglycan | peptidoglycan lytic | 1.00E−29 |
| | 2343 | 2344 | | protein P45, Listeria | |
| | | | | innocua . . . | |
| 1166 | Seq. ID No. | Seq. ID No. | Response regulator | Response regulator | 1.00E−42 |
| | 2345 | 2346 | | (CheY-like domain, | |
| | | | | HT, Clostrid . . . | |
| 1171c | Seq. ID No. | Seq. ID No. | membrane | ABC-type | 1.00E−20 |
| | 2363 | 2364 | | transmembrane | |
| | | | | transport protein, | |
| | | | | Campylob . . . | |
| 1177 | Seq. ID No. | Seq. ID No. | capsular | capsular | 3.00E−11 |
| | 2373 | 2374 | | polysaccharide | |
| | | | | biosynthesi . . . | |
| | | | | U67548 . . . | |
| 1180e | Seq. ID No. | Seq. ID No. | wall | unknown protein, | 3.00E−66 |
| | 2391 | 2392 | | Streptococcus | |
| | | | | mutans Bacteria; | |
| | | | | Fi . . . | |
| 1191 | Seq. ID No. | Seq. ID No. | transporter | putative ABC | 3.00E−70 |
| | 2417 | 2418 | | transporter (ATP- | |
| | | | | binding protein), | |
| | | | | St . . . | |
| 1197 | Seq. ID No. | Seq. ID No. | Periplasmic | Periplasmic serine | 2.00E−37 |
| | 2429 | 2430 | | proteases (ClpP | |
| | | | | class), Thermoa . . . | |
| 1214 | Seq. ID No. | Seq. ID No. | protease | Listeria innocua | 4.00E−35 |
| | 2465 | 2466 | | Bacteria; Firmicutes; | |
| | | | | Bacillus/Cl . . . | |
| 1218b | Seq. ID No. | Seq. ID No. | protease | glycoprotease family | 1.00E−115 |
| | 2475 | 2476 | | protein, | |
| | | | | Streptococcus | |
| | | | | pneumo . . . | |
| 1221b | Seq. ID No. | Seq. ID No. | transporter | hypothetical ABC | 1.00E−155 |
| | 2483 | 2484 | | transporter ATP- | |
| | | | | bindin, Staphyloc . . . | |
| 1233c | Seq. ID No. | Seq. ID No. | transporter | putative ATP-binding | 6.00E−26 |
| | 2513 | 2514 | | component of a | |
| | | | | transpor, Esch . . . | |
| 1237 | Seq. ID No. | Seq. ID No. | wall | YocH, Bacillus | 4.00E−14 |
| | 2525 | 2526 | | subtilis Bacteria; | |
| | | | | Firmicutes; Baci . . . | |
| 1258d | Seq. ID No. | Seq. ID No. | transporter | P-type ATPase - | 1.00E−159 |
| | 2589 | 2590 | | calcium transporter, | |
| | | | | Streptococcus . . . | |
| 1271 | Seq. ID No. | Seq. ID No. | transporter | Met, Streptococcus | 4.00E−99 |
| | 2613 | 2614 | | pneumoniae | |
| | | | | Bacteria; | |
| | | | | Firmicutes . . . | |
| 1288 | Seq. ID No. | Seq. ID No. | membrane | membrane bound | 3.00E−42 |
| | 2651 | 2652 | | protein, Bacillus | |
| | | | | subtilis Bacteria . . . | |
| 1300 | Seq. ID No. | Seq. ID No. | transport | secretory protein for | 1.00E−121 |
| | 2669 | 2670 | | transport of . . . | |
| | | | | X65961 . . . | |
| 1305c | Seq. ID No. | Seq. ID No. | transporter | cell division ABC | 7.00E−81 |
| | 2677 | 2678 | | transporter, ATP- | |
| | | | | bindin, Strepto . . . | |
| 1308e | Seq. ID No. | Seq. ID No. | transporter | cell division ABC | 4.00E−47 |
| | 2687 | 2688 | | transporter, | |
| | | | | permease protei, | |
| | | | | St . . . | |
| 1311 | Seq. ID No. | Seq. ID No. | response regulator | two-component | 3.00E−79 |
| | 2689 | 2690 | | response regulator | |
| | | | | involved i, Bacil . . . | |
| 1315 | Seq. ID No. | Seq. ID No. | Membrane | Membrane- | 1.00E−36 |
| | 2693 | 2694 | | associated sensory | |
| | | | | histi . . . AE007774 . . . | |
| 1319b | Seq. ID No. | Seq. ID No. | membrane | ABC transporter | 7.00E−90 |
| | 2703 | 2704 | | membrane-spanning | |
| | | | | permease , Strep . . . | |
| 1320c | Seq. ID No. | Seq. ID No. | transmembrane | phosphate ABC | 1.00E−78 |
| | 2709 | 2710 | | transporter, | |
| | | | | permease protein, | |
| | | | | Strep . . . | |
| 1322b | Seq. ID No. | Seq. ID No. | transport | Listeria | 1.00E−29 |
| | 2715 | 2716 | | monocytogenes | |
| | | | | Bacteria; Firmicutes; | |
| | | | | Bacil . . . | |
| 1344b | Seq. ID No. | Seq. ID No. | lipoprotein | putative HPr(ser) | 8.00E−79 |
| | 2751 | 2752 | | kinase, | |
| | | | | Streptococcus | |
| | | | | mutans Bac . . . | |
| 1348 | Seq. ID No. | Seq. ID No. | lipoprotein | Listeria | 2.00E−66 |
| | 2753 | 2754 | | monocytogenes | |
| | | | | Bacteria; Firmicutes; | |
| | | | | Bacil . . . | |
| 1355 | Seq. ID No. | Seq. ID No. | Capsular | Capsular | 1.00E−103 |
| | 2767 | 2768 | | polysaccharide of | |
| | | | | Streptoco . . . | |
| | | | | Aar83041 . . . | |
| 1366b | Seq. ID No. | Seq. ID No. | protease | protease ClpP, | 1.00E−81 |
| | 2787 | 2788 | | Listeria | |
| | | | | monocytogenes | |
| | | | | Bacteria; Fi . . . | |
| 1368 | Seq. ID No. | Seq. ID No. | receptor | plasmin receptor, | 1.00E−100 |
| | 2791 | 2792 | | Streptococcus | |
| | | | | pyogenes | |
| | | | | Bacteria; . . . | |
| 1377 | Seq. ID No. | Seq. ID No. | iron | enolase, | 2.00E−66 |
| | 2803 | 2804 | | Spironucleus vortens | |
| | | | | Euk . . . AF159517 . . . | |
| 1408 | Seq. ID No. | Seq. ID No. | transporter | ABC transporter, | 1.00E−116 |
| | 2867 | 2868 | | Lactobacillus | |
| | | | | plantarum | |
| | | | | Bacteria; . . . | |
| 1410 | Seq. ID No. | Seq. ID No. | transport | bacillus subtilis. | 1.00E−39 |
| | 2871 | 2872 | | hypothetical abc | |
| | | | | transporter at . . . | |
| 1426b | Seq. ID No. | Seq. ID No. | polysaccharide | Orfde2, | 3.00E−24 |
| | 2907 | 2908 | | Enterococcus | |
| | | | | faecalis Bacteria; | |
| | | | | Firmicutes . . . | |
| 1431 | Seq. ID No. | Seq. ID No. | transporter | Bacillus subtilis | 1.00E−100 |
| | 2919 | 2920 | | Bacteria; Firmicutes; | |
| | | | | Bacillus/C . . . | |
| 1432 | Seq. ID No. | Seq. ID No. | transporter | predicted membrane | 3.00E−38 |
| | 2921 | 2922 | | components of a, | |
| | | | | Thermoanaeroba . . . | |
| 1433 | Seq. ID No. | Seq. ID No. | immunogenic | Propionibacterium | 1.00E−111 |
| | 2923 | 2924 | | acnes immunogenic | |
| | | | | . . . Aau55208 . . . | |
| 1434b | Seq. ID No. | Seq. ID No. | iron | Bacillus subtilis | 8.00E−37 |
| | 2927 | 2928 | | Bacteria; Firmicutes; | |
| | | | | Bacillus/C . . . | |
| 1437c | Seq. ID No. | Seq. ID No. | transporter | ABC transporter | 1.00E−102 |
| | 2933 | 2934 | | subunit, | |
| | | | | Synechocys . . . | |
| | | | | D64004 . . . | |
| 1442b | Seq. ID No. | Seq. ID No. | transporter | ABC transporter | 2.00E−47 |
| | 2957 | 2958 | | (ATP-binding | |
| | | | | protein), Bacillus | |
| | | | | ha . . . | |
| 1451 | Seq. ID No. | Seq. ID No. | transporter | ABC transporter, | 1.00E−23 |
| | 2979 | 2980 | | ATP-binding protein, | |
| | | | | Streptococcu . . . | |
| 1457c | Seq. ID No. | Seq. ID No. | transporter | Na/Pi cotransporter | 1.00E−118 |
| | 2999 | 3000 | | II-related protein, | |
| | | | | Streptococ . . . | |
| 1462b | Seq. ID No. | Seq. ID No. | transporter | probable ABC | 1.00E−111 |
| | 3005 | 3006 | | transporter (ATP- | |
| | | | | binding protein), | |
| | | | | Ye . . . | |
| 1466d | Seq. ID No. | Seq. ID No. | membrane | ABC transporter | 1.00E−115 |
| | 3019 | 3020 | | ATP- | |
| | | | | binding/membrane | |
| | | | | spannin, Stre . . . | |
| 1470b | Seq. ID No. | Seq. ID No. | membrane | Integral membrane | 2.00E−11 |
| | 3031 | 3032 | | protein, | |
| | | | | Fusobacterium | |
| | | | | nucleatum . . . | |
| 1471 | Seq. ID No. | Seq. ID No. | channel | potassium channel | 6.00E−20 |
| | 3035 | 3036 | | protein, | |
| | | | | Methanosarcina | |
| | | | | acetivor . . . | |
| 1479c | Seq. ID No. | Seq. ID No. | wall | Listeria | 2.00E−15 |
| | 3055 | 3056 | | monocytogenes | |
| | | | | Bacteria; Firmicutes; | |
| | | | | Bacil . . . | |
| 1484c | Seq. ID No. | Seq. ID No. | transporter | magnesium (Mg2+) | 2.00E−65 |
| | 3067 | 3068 | | transporter, Bacillus | |
| | | | | halodurans . . . | |
| 1488g | Seq. ID No. | Seq. ID No. | transporter | Staphylococcus | 2.00E−55 |
| | 3085 | 3086 | | aureus subsp. | |
| | | | | aureus N315 | |
| | | | | Bacteria; . . . | |
| 1530b | Seq. ID No. | Seq. ID No. | peptidoglycan | Penicillin-binding | 1.00E−120 |
| | 3165 | 3166 | | protein 1b, | |
| | | | | Streptococcus | |
| | | | | pneum . . . | |
| 1543 | Seq. ID No. | Seq. ID No. | invasion | extracellular protein, | 2.00E−26 |
| | 3183 | 3184 | | Listeria welshimeri | |
| | | | | Bacteri . . . | |
| 1547b | Seq. ID No. | Seq. ID No. | spore | Bacillus subtilis | 2.00E−71 |
| | 3189 | 3190 | | Bacteria; Firmicutes; | |
| | | | | Bacillus/C . . . | |
| 1553b | Seq. ID No. | Seq. ID No. | transporter | putative ABC | 3.00E−55 |
| | 3197 | 3198 | | transporter subunit | |
| | | | | ComYA, | |
| | | | | Streptococ . . . | |
| 1556c | Seq. ID No. | Seq. ID No. | transporter | Na+ ABC transporter | 1.00E−36 |
| | 3211 | 3212 | | (ATP-binding | |
| | | | | protein), NATA, C . . . | |
| 1573 | Seq. ID No. | Seq. ID No. | transporter | ABC transporter, | 2.00E−42 |
| | 3251 | 3252 | | ATP-binding protein, | |
| | | | | Streptococcu . . . | |
| 1577b | Seq. ID No. | Seq. ID No. | transport | drug efflux ABC | 7.00E−12 |
| | 3257 | 3258 | | transporter, | |
| | | | | Streptococcus | |
| | | | | pneumon . . . | |
| 1578 | Seq. ID No. | Seq. ID No. | transport | ABC transporter, | 7.00E−34 |
| | 3259 | 3260 | | Lactobacillus | |
| | | | | helveticus | |
| | | | | Bacteria . . . | |
| 1594a | Seq. ID No. | Seq. ID No. | transmembrane | Chlamydia | 9.00E−22 |
| | 3297 | 3298 | | pneumoniae | |
| | | | | transmembrane p . . . | |
| | | | | Aay35379 . . . | |
| 1606b | Seq. ID No. | Seq. ID No. | response regulator | putative histidine | 4.00E−69 |
| | 3319 | 3320 | | protein kinase, | |
| | | | | Lactococcus lac . . . | |
| 1607b | Seq. ID No. | Seq. ID No. | lipoprotein | YqjG, Bacillus | 7.00E−30 |
| | 3323 | 3324 | | subtilis Bacteria; | |
| | | | | Firmicutes; Baci . . . | |
| 1616b | Seq. ID No. | Seq. ID No. | sporulation | Staphylococcus | 1.00E−156 |
| | 3335 | 3336 | | aureus subsp. | |
| | | | | aureus MW2 | |
| | | | | Bacteria; . . . | |
| 1619 | Seq. ID No. | Seq. ID No. | lipoprotein | CD4+ T cell- | 4.00E−81 |
| | 3337 | 3338 | | stimulating antigen, | |
| | | | | lipoprotein, List . . . | |
| 1625c | Seq. ID No. | Seq. ID No. | transport | putative sugar ABC | 3.00E−76 |
| | 3355 | 3356 | | transporter | |
| | | | | (permeas, | |
| | | | | Streptoco . . . | |
| 1626b | Seq. ID No. | Seq. ID No. | membrane | sugar ABC | 7.00E−97 |
| | 3359 | 3360 | | transporter, | |
| | | | | permease protein, | |
| | | | | Streptoco . . . | |
| 1634c | Seq. ID No. | Seq. ID No. | peptidoglycan | beta-1,4-N- | 4.00E−34 |
| | 3377 | 3378 | | acetylmuramoylhydro- | |
| | | | | lase, Enterococcus | |
| | | | | h . . . | |
| 1646e | Seq. ID No. | Seq. ID No. | polysaccharide | putative | 3.00E−78 |
| | 3397 | 3398 | | polysaccharide | |
| | | | | biosynthesis protein, | |
| | | | | Stre . . . | |
| 1649a | Seq. ID No. | Seq. ID No. | penicillin | Streptococcus | 7.00E−35 |
| | 3405 | 3406 | | thermophilus | |
| | | | | Bacteria; Firmicutes; | |
| | | | | G . . . | |
| 1654 | Seq. ID No. | Seq. ID No. | immunogenic | Propionibacterium | 8.00E−24 |
| | 3415 | 3416 | | acnes immunogenic | |
| | | | | . . . Aau49489 . . . | |
| 1659c | Seq. ID No. | Seq. ID No. | membrane | putative integral | 2.00E−93 |
| | 3425 | 3426 | | membrane protein, | |
| | | | | Campylobacter . . . | |
| 1660b | Seq. ID No. | Seq. ID No. | membrane | putative integral | 1.00E−24 |
| | 3427 | 3428 | | membrane protein, | |
| | | | | Campylobacter . . . | |
| 1674 | Seq. ID No. | Seq. ID No. | membrane | putative integral | 9.00E−15 |
| | 3461 | 3462 | | membrane protein, | |
| | | | | Campylobacter . . . | |
| 1675a | Seq. ID No. | Seq. ID No. | immunogenic | Propionibacterium | 3.00E−24 |
| | 3463 | 3464 | | acnes immunogenic | |
| | | | | . . . Aau47623 . . . | |
| 1685 | Seq. ID No. | Seq. ID No. | immunogenic | Propionibacterium | 2.00E−50 |
| | 3487 | 3488 | | acnes immunogenic | |
| | | | | . . . Aau58232 . . . | |
| 1688b | Seq. ID No. | Seq. ID No. | polysaccharide | Orfde3, | 5.00E−87 |
| | 3493 | 3494 | | Enterococcus | |
| | | | | faecalis Bacteria; | |
| | | | | Firmicutes . . . | |
| 1689a | Seq. ID No. | Seq. ID No. | cps7H | putative | 5.00E−38 |
| | 3497 | 3498 | | glycosyltransferase | |
| | | | | Cps7H, | |
| | | | | Streptococcus . . . | |
| 1691 | Seq. ID No. | Seq. ID No. | membrane | Hypothetical | 6.00E−12 |
| | 3507 | 3508 | | membrane-spanning | |
| | | | | protein, | |
| | | | | Fusobacter . . . | |
| 1694d | Seq. ID No. | Seq. ID No. | membrane | membrane bound | 1.00E−68 |
| | 3519 | 3520 | | protein, Bacillus | |
| | | | | subtilis Bacteria . . . | |
| 1696 | Seq. ID No. | Seq. ID No. | transport | UDP-Glc-6- | 1.00E−155 |
| | 3521 | 3522 | | dehydrogenase Ugd, | |
| | | | | Escherichia coli | |
| | | | | Bact . . . | |
| 1699b | Seq. ID No. | Seq. ID No. | transport | probable export | 4.00E−28 |
| | 3529 | 3530 | | protein, Escherichia | |
| | | | | coli K12 Bact . . . | |
| 1701a | Seq. ID No. | Seq. ID No. | immunogenic | Propionibacterium | 4.00E−34 |
| | 3535 | 3536 | | acnes immunogenic | |
| | | | | . . . Aau67497 . . . | |
| 1752b | Seq. ID No. | Seq. ID No. | surface | streptococcal | 7.00E−14 |
| | 3629 | 3630 | | surface protein, | |
| | | | | Streptococcus | |
| | | | | dysga . . . | |
| 1756b | Seq. ID No. | Seq. ID No. | membrane | hypothetical | 1.00E−31 |
| | 3635 | 3636 | | membrane protein | |
| | | | | LaaO, Lactobacillus | |
| | | | | . . . | |
| 1798c | Seq. ID No. | Seq. ID No. | membrane | Uncharacterized | 1.00E−13 |
| | 3727 | 3728 | | membrane protein, | |
| | | | | homolog o, Clost . . . | |
| 1808d | Seq. ID No. | Seq. ID No. | membrane | mbh8 NADH | 7.00E−43 |
| | 3759 | 3760 | | dehydrogenase | |
| | | | | subunit - integra, | |
| | | | | Pyrococ . . . | |
| 1809f | Seq. ID No. | Seq. ID No. | membrane | mbh8 NADH | 2.00E−48 |
| | 3771 | 3772 | | dehydrogenase | |
| | | | | subunit - integra, | |
| | | | | Pyrococ . . . | |
| 1816a | Seq. ID No. | Seq. ID No. | peptidoglycan | Penicillin-binding | 1.00E−124 |
| | 3793 | 3794 | | protein 1A, | |
| | | | | Streptococcus | |
| | | | | pneum . . . | |
| 1838b | Seq. ID No. | Seq. ID No. | transport | ABC-type | 6.00E−57 |
| | 3839 | 3840 | | multidrug/protein/lipid | |
| | | | | transpor, | |
| | | | | Thermoa . . . | |
| 1842b | Seq. ID No. | Seq. ID No. | protease | sac operon related | 1.00E−22 |
| | 3851 | 3852 | | regulation protein, | |
| | | | | Bacillus sp . . . | |
| 1845c | Seq. ID No. | Seq. ID No. | channel | voltage-gated | 4.00E−63 |
| | 3863 | 3864 | | chloride channel | |
| | | | | family protein, Str . . . | |
| 1848a | Seq. ID No. | Seq. ID No. | membrane | Integral membrane | 6.00E−24 |
| | 3865 | 3866 | | protein, | |
| | | | | Fusobacterium | |
| | | | | nucleatum . . . | |
| 1855 | Seq. ID No. | Seq. ID No. | capsule | Regulatory function | 1.00E−13 |
| | 3883 | 3884 | | on capsule ex . . . | |
| | | | | AE008458 . . . | |
| 1857c | Seq. ID No. | Seq. ID No. | polysaccharide | Staphylococcus | 7.00E−15 |
| | 3891 | 3892 | | aureus subsp. | |
| | | | | aureus N315 | |
| | | | | Bacteria; . . . | |
| 1865b | Seq. ID No. | Seq. ID No. | membrane | Listeria innocua | 2.00E−40 |
| | 3913 | 3914 | | Bacteria; Firmicutes; | |
| | | | | Bacillus/Cl . . . | |
| 1873c | Seq. ID No. | Seq. ID No. | protease | OrfRM1, Bacillus | 8.00E−91 |
| | 3925 | 3926 | | subtilis Bacteria; | |
| | | | | Firmicutes; GE . . . | |
| 1875c | Seq. ID No. | Seq. ID No. | Signal | ref\|NP_655047.1\| | 1.00E−32 |
| | 3929 | 3930 | | (NC_003995) | |
| | | | | Peptidase_S26, | |
| | | | | Signal . . . | |
| 1898b | Seq. ID No. | Seq. ID No. | membrane | Hypothetical | 6.00E−32 |
| | 3975 | 3976 | | membrane-spanning | |
| | | | | protein, | |
| | | | | Fusobacter . . . | |
| 1902a | Seq. ID No. | Seq. ID No. | capsular | DNA topoisomerase | 0 |
| | 3981 | 3982 | | IV subunit B, | |
| | | | | Streptococcus | |
| | | | | agal . . . | |
| 1931b | Seq. ID No. | Seq. ID No. | transport | transporter protein, | 3.00E−22 |
| | 4027 | 4028 | | Haemophilus | |
| | | | | influenzae Rd Bac . . . | |
| 1936 | Seq. ID No. | Seq. ID No. | transport | Bacillus subtilis | 2.00E−18 |
| | 4039 | 4040 | | Bacteria; Firmicutes; | |
| | | | | Bacillus/C . . . | |
| 1941b | Seq. ID No. | Seq. ID No. | Membrane | Membrane | 6.00E−18 |
| | 4061 | 4062 | | associated histidine | |
| | | | | kinase-lik, Clostrid . . . | |
| 1946b | Seq. ID No. | Seq. ID No. | transport | probable amino acid | 2.00E−56 |
| | 4073 | 4074 | | transport protein, | |
| | | | | Clostridium . . . | |
| 1951a | Seq. ID No. | Seq. ID No. | membrane | ABC-type | 1.00E−137 |
| | 4081 | 4082 | | multidrug/protein/lipid | |
| | | | | transpor, Clostri . . . | |
| 1958b | Seq. ID No. | Seq. ID No. | membrane | ABC transporter | 8.00E−44 |
| | 4103 | 4104 | | membrane-spanning | |
| | | | | permease, Strep . . . | |
| 1960c | Seq. ID No. | Seq. ID No. | transport | ABC transporter | 3.00E−68 |
| | 4111 | 4112 | | substrate-binding | |
| | | | | protein, Strept . . . | |
| 1973 | Seq. ID No. | Seq. ID No. | peptidoglycan | peptidoglycan N- | 1.00E−15 |
| | 4137 | 4138 | | acetylglucosamine | |
| | | | | deacetylase A, S . . . | |
| 2016c | Seq. ID No. | Seq. ID No. | transport | Listeria | 2.00E−20 |
| | 4225 | 4226 | | monocytogenes | |
| | | | | Bacteria; Firmicutes; | |
| | | | | Bacil . . . | |
| 2024b | Seq. ID No. | Seq. ID No. | penicillin | Listeria | 1.00E−132 |
| | 4247 | 4248 | | monocytogenes | |
| | | | | Bacteria; Firmicutes; | |
| | | | | Bacil . . . | |
| 2074b | Seq. ID No. | Seq. ID No. | wall | Bacillus subtilis | 2.00E−26 |
| | 4343 | 4344 | | Bacteria; Firmicutes; | |
| | | | | Bacillus/C . . . | |
| 2092 | Seq. ID No. | Seq. ID No. | response regulator | Hpk1, Streptococcus | 2.00E−22 |
| | 4379 | 4380 | | thermophilus | |
| | | | | Bacteria; Firmicu . . . | |
| 2096c | Seq. ID No. | Seq. ID No. | transport | Staphylococcus | 1.00E−61 |
| | 4389 | 4390 | | aureus subsp. | |
| | | | | aureus MW2 | |
| | | | | Bacteria; . . . | |
| 2140a | Seq. ID No. | Seq. ID No. | protease | Listeria innocua | 6.00E−55 |
| | 4469 | 4470 | | Bacteria; Firmicutes; | |
| | | | | Bacillus/Cl . . . | |
| 2142a | Seq. ID No. | Seq. ID No. | phosphatidate | putative | 1.00E−11 |
| | 4473 | 4474 | | phosphatidate | |
| | | | | cytidylyltransferas, | |
| | | | | Strept . . . | |
| 2146 | Seq. ID No. | Seq. ID No. | lipoprotein | Listeria innocua | 1.00E−68 |
| | 4481 | 4482 | | Bacteria; Firmicutes; | |
| | | | | Bacillus/Cl . . . | |
| 2147b | Seq. ID No. | Seq. ID No. | transport | Listeria | 5.00E−41 |
| | 4485 | 4486 | | monocytogenes | |
| | | | | Bacteria; Firmicutes; | |
| | | | | Bacil . . . | |
| 2150a | Seq. ID No. | Seq. ID No. | immunogenic | Propionibacterium | 9.00E−15 |
| | 4493 | 4494 | | acnes immunogenic | |
| | | | | protein #18988 . . . | |
| 2161c | Seq. ID No. | Seq. ID No. | immunogenic | Propionibacterium | 5.00E−12 |
| | 4529 | 4530 | | acnes immunogenic | |
| | | | | protein #20073 . . . | |
| 2219c | Seq. ID No. | Seq. ID No. | membrane | Conserved | 4.00E−20 |
| | 4643 | 4644 | | membrane protein, | |
| | | | | probabl, Clostridium | |
| | | | | a . . . | |
| 2249 | Seq. ID No. | Seq. ID No. | transport | oligopeptide ABC | 1.00E−148 |
| | 4705 | 4706 | | transporte, Bacillus | |
| | | | | halodurans B . . . | |
| 2253d | Seq. ID No. | Seq. ID No. | transport | oligopeptide ABC | 1.00E−60 |
| | 4713 | 4714 | | transporter | |
| | | | | (permease), Bacillus . . . | |
| 2255b | Seq. ID No. | Seq. ID No. | transport | oligopeptide ABC | 8.00E−93 |
| | 4717 | 4718 | | transporter | |
| | | | | (permease), Bacillus . . . | |
| 2300 | Seq. ID No. | Seq. ID No. | transport | H+-transporting ATP | 2.00E−18 |
| | 4813 | 4814 | | synthase, subunit K | |
| | | | | (atpK), Me . . . | |
| 2301 | Seq. ID No. | Seq. ID No. | transport | H+-transporting ATP | 3.00E−43 |
| | 4815 | 4816 | | synthase, subunit I | |
| | | | | (atpI), Py . . . | |
| 2307 | Seq. ID No. | Seq. ID No. | membrane | membrane | 1.00E−55 |
| | 4825 | 4826 | | permease OpuCD, | |
| | | | | Listeria | |
| | | | | monocytogenes | |
| | | | | Ba . . . | |
| 2308 | Seq. ID No. | Seq. ID No. | transport | Listeria innocua | 5.00E−81 |
| | 4827 | 4828 | | Bacteria; Firmicutes; | |
| | | | | Bacillus/Cl . . . | |
| 2309c | Seq. ID No. | Seq. ID No. | transport | membrane | 9.00E−33 |
| | 4833 | 4834 | | permease OpuCB, | |
| | | | | Listeria | |
| | | | | monocytogenes | |
| | | | | Ba . . . | |
| 2311 | Seq. ID No. | Seq. ID No. | transport | glycine | 1.00E−109 |
| | 4837 | 4838 | | betaine/carnitine/ | |
| | | | | choline AB, | |
| | | | | Staphylococc . . . | |
| 2317 | Seq. ID No. | Seq. ID No. | immunogenic | Propionibacterium | 9.00E−17 |
| | 4851 | 4852 | | acnes immunogenic | |
| | | | | protein #7465 . . . . | |
| 2324 | Seq. ID No. | Seq. ID No. | immunogenic | Propionibacterium | 5.00E−18 |
| | 4863 | 4864 | | acnes immunogenic | |
| | | | | protein #5020 . . . . | |
| 2329d | Seq. ID No. | Seq. ID No. | membrane | Listeria innocua | 2.00E−72 |
| | 4875 | 4876 | | Bacteria; Firmicutes; | |
| | | | | Bacillus/Cl . . . | |
| 2331 | Seq. ID No. | Seq. ID No. | membrane | Listeria | 1.00E−28 |
| | 4879 | 4880 | | monocytogenes | |
| | | | | Bacteria; Firmicutes; | |
| | | | | Bacil . . . | |
| 2338c | Seq. ID No. | Seq. ID No. | membrane | predicted membrane | 1.00E−178 |
| | 4893 | 4894 | | GTPase involved in | |
| | | | | stres, Therm . . . | |
| 2344 | Seq. ID No. | Seq. ID No. | membrane | dihydrolipoamide | 1.00E−167 |
| | 4901 | 4902 | | dehydrogenase | |
| | | | | component o, | |
| | | | | Staphy . . . | |
| 2357 | Seq. ID No. | Seq. ID No. | sensor protein | sensor protein | 1.00E−47 |
| | 4919 | 4920 | | kinase, Lactococcus | |
| | | | | lactis subsp. l . . . | |
| 2363b | Seq. ID No. | Seq. ID No. | periplasmic | predicted | 4.00E−45 |
| | 4931 | 4932 | | periplasmic solute- | |
| | | | | binding protein, | |
| | | | | Ther . . . | |
| 2376 | Seq. ID No. | Seq. ID No. | virulence | aspartate | 1.00E−117 |
| | 4959 | 4960 | | semialdehyde | |
| | | | | dehydrogenase, | |
| | | | | Staphylococc . . . | |
| 2377b | Seq. ID No. | Seq. ID No. | virulence | Staphylococcus | 2.00E−87 |
| | 4963 | 4964 | | aureus mutant | |
| | | | | P14C15 virulence | |
| | | | | gene . . . | |
| 2387 | Seq. ID No. | Seq. ID No. | Transport | Transporter, | 9.00E−40 |
| | 4985 | 4986 | | Fusobacterium | |
| | | | | nucleatum subsp. | |
| | | | | nuclea . . . | |
| 2452 | Seq. ID No. | Seq. ID No. | sporulation | alanine | 1.00E−118 |
| | 5113 | 5114 | | dehydrogenase, | |
| | | | | Bacillus subtilis | |
| | | | | Bacteria; . . . | |
| 2453 | Seq. ID No. | Seq. ID No. | ferric | Bacillus subtilis | 5.00E−48 |
| | 5115 | 5116 | | Bacteria; Firmicutes; | |
| | | | | Bacillus/C . . . | |
| 2455 | Seq. ID No. | Seq. ID No. | ferric | Bacillus subtilis | 2.00E−76 |
| | 5117 | 5118 | | Bacteria; Firmicutes; | |
| | | | | Bacillus/C . . . | |
| 2456d | Seq. ID No. | Seq. ID No. | transport | Bacillus subtilis | 2.00E−54 |
| | 5125 | 5126 | | Bacteria; Firmicutes; | |
| | | | | Bacillus/C . . . | |
| 2458g | Seq. ID No. | Seq. ID No. | transporter | ferrichrome ABC | 2.00E−65 |
| | 5141 | 5142 | | transporter | |
| | | | | (permease), Bacillus | |
| | | | | h . . . | |
| 2475c | Seq. ID No. | Seq. ID No. | secreted protein | Human secreted | 1.00E−109 |
| | 5183 | 5184 | | protein sequence | |
| | | | | enco . . . | |
| | | | | Aab34842 . . . | |
| 2485 | Seq. ID No. | Seq. ID No. | wall | cell division protein, | 1.00E−114 |
| | 5201 | 5202 | | Enterococcus | |
| | | | | faecalis Bacte . . . | |
| 2490 | Seq. ID No. | Seq. ID No. | wall | cell division protein, | 5.00E−22 |
| | 5205 | 5206 | | Enterococcus | |
| | | | | faecalis Bacte . . . | |
| 2495a | Seq. ID No. | Seq. ID No. | penicillin | MRAY, | 6.00E−64 |
| | 5213 | 5214 | | Staphylococcus | |
| | | | | aureus Bacteria; | |
| | | | | Firmicutes; . . . | |
| 2497b | Seq. ID No. | Seq. ID No. | wall | penicillin-binding | 1.00E−116 |
| | 5219 | 5220 | | protein, | |
| | | | | Enterococcus | |
| | | | | faecalis . . . | |
| 2520d | Seq. ID No. | Seq. ID No. | phosphatase | phosphoenolpyruvate- | 0 |
| | 5279 | 5280 | | protein | |
| | | | | phosphatase, | |
| | | | | Staphyloc . . . | |
| 2525a | Seq. ID No. | Seq. ID No. | virulence | Amino acid | 3.00E−11 |
| | 5285 | 5286 | | sequence of a | |
| | | | | virulence factor | |
| | | | | encoded . . . | |
| 2547c | Seq. ID No. | Seq. ID No. | immunogenic | Propionibacterium | 2.00E−14 |
| | 5331 | 5332 | | acnes immunogenic | |
| | | | | protein #22248 . . . | |
| 2564d | Seq. ID No. | Seq. ID No. | transport | carboxypeptidase, | 1.00E−42 |
| | 5381 | 5382 | | Bacillus subtilis | |
| | | | | Bacteria; Firm . . . | |
| 2568 | Seq. ID No. | Seq. ID No. | transport | ABC-type multidrug | 2.00E−47 |
| | 5389 | 5390 | | transport system, | |
| | | | | ATPas, Thermo . . . | |
| 2582a | Seq. ID No. | Seq. ID No. | membrane | ABC transporter | 9.00E−66 |
| | 5405 | 5406 | | membrane-spanning | |
| | | | | permease, Strep . . . | |
| 2585 | Seq. ID No. | Seq. ID No. | transport | ABC transporter | 1.00E−148 |
| | 5411 | 5412 | | ATP-binding protein - | |
| | | | | cobal, Strep . . . | |
| 2589 | Seq. ID No. | Seq. ID No. | lipoprotein | Chlamydia | 1.00E−120 |
| | 5419 | 5420 | | trachomatis | |
| | | | | lipoprotein se . . . | |
| | | | | Aay37627 . . . | |
| 2596 | Seq. ID No. | Seq. ID No. | immunogenic | Propionibacterium | 2.00E−27 |
| | 5433 | 5434 | | acnes immunogenic | |
| | | | | protein #4493 . . . . | |
| 2616c | Seq. ID No. | Seq. ID No. | membrane | Listeria innocua | 1.00E−10 |
| | 5463 | 5464 | | Bacteria; Firmicutes; | |
| | | | | Bacillus/Cl . . . | |
| 2617b | Seq. ID No. | Seq. ID No. | transport | putative ABC | 9.00E−69 |
| | 5477 | 5478 | | transporter (ATP- | |
| | | | | binding protein), | |
| | | | | St . . . | |
| 2623b | Seq. ID No. | Seq. ID No. | wall | Lactococcus lactis | 2.00E−23 |
| | 5491 | 5492 | | subsp. cremoris | |
| | | | | Bacteria; Firmi . . . | |
| 2628 | Seq. ID No. | Seq. ID No. | transport | Pseudogene, similar | 2.00E−77 |
| | 5509 | 5510 | | to glycine-betaine | |
| | | | | bindin, Lis . . . | |
| 2629b | Seq. ID No. | Seq. ID No. | transport | oligopeptide ABC | 1.00E−149 |
| | 5513 | 5514 | | transporte, Bacillus | |
| | | | | halodurans B . . . | |
| 2633d | Seq. ID No. | Seq. ID No. | transport | oligopeptide ABC | 1.00E−149 |
| | 5525 | 5526 | | transporte, Bacillus | |
| | | | | halodurans B . . . | |
| 2637a | Seq. ID No. | Seq. ID No. | transport | oligopeptide ABC | 1.00E−140 |
| | 5529 | 5530 | | transporte, Bacillus | |
| | | | | halodurans B . . . | |
| 2643b | Seq. ID No. | Seq. ID No. | peptidoglycan | Penicillin-binding | 1.00E−143 |
| | 5537 | 5538 | | protein 2a, | |
| | | | | Streptococcus | |
| | | | | pneum . . . | |
| 2659b | Seq. ID No. | Seq. ID No. | hemolysin | putative hemolysin, | 5.00E−71 |
| | 5567 | 5568 | | Streptococcus | |
| | | | | pyogenes | |
| | | | | MGAS823 . . . | |
| 2674b | Seq. ID No. | Seq. ID No. | membrane | putative integral | 5.00E−21 |
| | 5599 | 5600 | | membrane protei . . . | |
| | | | | AL162756 . . . | |
| 2680c | Seq. ID No. | Seq. ID No. | virulence | Staphylococcus | 1.00E−160 |
| | 5621 | 5622 | | aureus mutant | |
| | | | | P11C12 virulence | |
| | | | | gene . . . | |
| 2688c | Seq. ID No. | Seq. ID No. | transport | phosphotidylglycero | 4.00E−43 |
| | 5637 | 5638 | | phosphate synthase, | |
| | | | | Streptococc . . . | |
| 2723 | Seq. ID No. | Seq. ID No. | membrane | Uncharacterized | 1.00E−13 |
| | 5699 | 5700 | | membrane protein, | |
| | | | | homolog o, Clost . . . | |
| 2734c | Seq. ID No. | Seq. ID No. | transport | cation transporter, | 9.00E−62 |
| | 5723 | 5724 | | Lactococcus lactis | |
| | | | | subsp. lact . . . | |
| 2738c | Seq. ID No. | Seq. ID No. | transport | transporter, Bacillus | 3.00E−21 |
| | 5733 | 5734 | | halodurans Bacteria; | |
| | | | | Firmicu . . . | |
| 2748c | Seq. ID No. | Seq. ID No. | transport | conserved | 5.00E−34 |
| | 5761 | 5762 | | hypothetical protein, | |
| | | | | Thermotoga | |
| | | | | maritim . . . | |
| 2758d | Seq. ID No. | Seq. ID No. | immunogenic | Propionibacterium | 3.00E−36 |
| | 5791 | 5792 | | acnes immunogenic | |
| | | | | . . . Aau49434 . . . | |
| 2801b | Seq. ID No. | Seq. ID No. | response regulator | VanSB, | 1.00E−21 |
| | 5895 | 5896 | | Enterococcus | |
| | | | | faecalis Bacteria; | |
| | | | | Firmicutes; . . . | |
| 2805b | Seq. ID No. | Seq. ID No. | transmembrane | transmembrane | 1.00E−130 |
| | 5901 | 5902 | | protein Vexp3, | |
| | | | | Streptococcus | |
| | | | | pneumon . . . | |
| 2809 | Seq. ID No. | Seq. ID No. | transport | ABC transporter, | 1.00E−63 |
| | 5903 | 5904 | | ATP-binding protein | |
| | | | | Vexp2, Strept . . . | |
| 2810c | Seq. ID No. | Seq. ID No. | membrane | ABG transporter | 1.00E−59 |
| | 5909 | 5910 | | membrane-spanning | |
| | | | | permease - Pe, S . . . | |
| 2812 | Seq. ID No. | Seq. ID No. | transport | probable ABC | 1.00E−102 |
| | 5913 | 5914 | | transporter, | |
| | | | | Clostridium | |
| | | | | perfringens . . . | |
| 2816 | Seq. ID No. | Seq. ID No. | transport | Listeria innocua | 5.00E−59 |
| | 5919 | 5920 | | Bacteria; Firmicutes; | |
| | | | | Bacillus/Cl . . . | |
| 2818e | Seq. ID No. | Seq. ID No. | transport | amino acid ABC | 2.00E−58 |
| | 5929 | 5930 | | transporter | |
| | | | | permease protein, | |
| | | | | Lacto . . . | |
| 2822a | Seq. ID No. | Seq. ID No. | membrane | putative membrane | 7.00E−27 |
| | 5939 | 5940 | | protein, Salmonella | |
| | | | | enterica sub . . . | |
| 2856c | Seq. ID No. | Seq. ID No. | transport | heavy-metal | 1.00E−131 |
| | 6001 | 6002 | | transporting CPx- | |
| | | | | type ATPase, | |
| | | | | Methanot . . . | |
| 2881 | Seq. ID No. | Seq. ID No. | surface | surface protein (GW | 4.00E−23 |
| | 6043 | 6044 | | repeat) similar t, | |
| | | | | Listeria in . . . | |
| 2883d | Seq. ID No. | Seq. ID No. | transport | ABC transporter | 1.00E−107 |
| | 6053 | 6054 | | permease protein, | |
| | | | | Lactococcus lact . . . | |
| 2886b | Seq. ID No. | Seq. ID No. | transport | Listeria | 5.00E−69 |
| | 6057 | 6058 | | monocytogenes | |
| | | | | Bacteria; Firmicutes; | |
| | | | | Bacil . . . | |
| 2900b | Seq. ID No. | Seq. ID No. | wall | YojL, Bacillus subtilis | 2.00E−15 |
| | 6089 | 6090 | | Bacteria; Firmicutes; | |
| | | | | Baci . . . | |
| 2902 | Seq. ID No. | Seq. ID No. | peptidoglycan | beta-1,4-N- | 2.00E−32 |
| | 6095 | 6096 | | acetylmuramoylhydro- | |
| | | | | lase, Enterococcus | |
| | | | | h . . . | |
| 2906 | Seq. ID No. | Seq. ID No. | immunoglobulin | Streptococcus sp. | 2.00E−17 |
| | 6103 | 6104 | | G148 Bacteria; | |
| | | | | Firmicutes; Bacil . . . | |
| 2909d | Seq. ID No. | Seq. ID No. | transmembrane | Listeria innocua | 1.00E−144 |
| | 6111 | 6112 | | Bacteria; Firmicutes; | |
| | | | | Bacillus/Cl . . . | |
| 2913b | Seq. ID No. | Seq. ID No. | response regulator | putative histidine | 1.00E−92 |
| | 6117 | 6118 | | kinase, Lactobacillus | |
| | | | | sakei Bac . . . | |
| 2931c | Seq. ID No. | Seq. ID No. | transport | putative | 8.00E−96 |
| | 6165 | 6166 | | sodium/glucose | |
| | | | | cotransporter, | |
| | | | | Salmonella . . . | |
| 2933b | Seq. ID No. | Seq. ID No. | immunogenic | Propionibacterium | 1.00E−19 |
| | 6169 | 6170 | | acnes immunogenic | |
| | | | | protein #6411 . . . . | |
| 2941b | Seq. ID No. | Seq. ID No. | transport | probable ABC | 1.00E−145 |
| | 6185 | 6186 | | transporter, | |
| | | | | Clostridium | |
| | | | | perfringens . . . | |
| 2943b | Seq. ID No. | Seq. ID No. | transport | ABC transporter | 1.00E−114 |
| | 6191 | 6192 | | ATP binding protein, | |
| | | | | Lactococcus l . . . | |
| 2948d | Seq. ID No. | Seq. ID No. | membrane | Listeria innocua | 4.00E−56 |
| | 6209 | 6210 | | Bacteria; Firmicutes; | |
| | | | | Bacillus/Cl . . . | |
| 2955e | Seq. ID No. | Seq. ID No. | membrane | putative integral | 4.00E−35 |
| | 6229 | 6230 | | membrane | |
| | | | | transporter protein, | |
| | | | | Si . . . | |
| 2965c | Seq. ID No. | Seq. ID No. | hemolysin | hemolysin, | 1E−55 |
| | 6257 | 6258 | | Treponema | |
| | | | | denticola Bact . . . | |
| | | | | U30249 . . . | |
| 2969 | Seq. ID No. | Seq. ID No. | Membrane | Membrane-fusion | 5.00E−23 |
| | 6263 | 6264 | | protein, | |
| | | | | Thermoanaerobacter | |
| | | | | tengco . . . | |
| 2971b | Seq. ID No. | Seq. ID No. | lipoprotein | ABC-type transport | 5.00E−72 |
| | 6271 | 6272 | | systems, involved i, | |
| | | | | Thermoanae . . . | |
| 2974b | Seq. ID No. | Seq. ID No. | lipoprotein | ABC-type transport | 4.00E−61 |
| | 6275 | 6276 | | systems, involved i, | |
| | | | | Thermoanae . . . | |
| 2980a | Seq. ID No. | Seq. ID No. | transport | unknown, | 5.00E−13 |
| | 6289 | 6290 | | Pasteurella | |
| | | | | multocida Bacteria; | |
| | | | | Proteobac . . . | |
| 2986b | Seq. ID No. | Seq. ID No. | transport | rnfD protein, | 3.00E−28 |
| | 6303 | 6304 | | Pseudomonas | |
| | | | | stutzeri A15 | |
| | | | | Bacteria; P . . . | |
| 3016b | Seq. ID No. | Seq. ID No. | membrane | ABC transporter | 3.00E−27 |
| | 6365 | 6366 | | membrane-spanning | |
| | | | | permease - Na, S . . . | |
| 3017 | Seq. ID No. | Seq. ID No. | transporter | ABC transporter | 6.00E−82 |
| | 6369 | 6370 | | ATP binding protein, | |
| | | | | Lactococcus l . . . | |
| 3024a | Seq. ID No. | Seq. ID No. | virulence | Amino acid | 2.00E−13 |
| | 6383 | 6384 | | sequence of a | |
| | | | | virulence factor | |
| | | | | encoded . . . | |
| 3037a | Seq. ID No. | Seq. ID No. | lipoprotein | putative host cell | 8.00E−13 |
| | 6415 | 6416 | | surface-exposed | |
| | | | | lipoprotein, St . . . | |

Listed in Table 9 are 242 ORFs that share some homology with the ORFs predicted by proteomic methods used for studying surface exposed proteins of Streptococcus pneumoniae.

TABLE 9
PROTEOMICS

| ORF Number | DNA SEQ ID Number | Protein SEQ ID Number |
|---|---|---|
| 1 | Seq. ID No. 1 | Seq. ID No. 2 |
| 2b | Seq. ID No. 5 | Seq. ID No. 6 |
| 56b | Seq. ID No. 93 | Seq. ID No. 94 |
| 57a | Seq. ID No. 95 | Seq. ID No. 96 |
| 60b | Seq. ID No. 103 | Seq. ID No. 104 |
| 62a | Seq. ID No. 107 | Seq. ID No. 108 |
| 85c | Seq. ID No. 157 | Seq. ID No. 158 |
| 112b | Seq. ID No. 223 | Seq. ID No. 224 |
| 114 | Seq. ID No. 225 | Seq. ID No. 226 |
| 121 | Seq. ID No. 233 | Seq. ID No. 234 |
| 124c | Seq. ID No. 239 | Seq. ID No. 240 |
| 129c | Seq. ID No. 253 | Seq. ID No. 254 |
| 157b | Seq. ID No. 317 | Seq. ID No. 318 |
| 158b | Seq. ID No. 321 | Seq. ID No. 322 |
| 165a | Seq. ID No. 341 | Seq. ID No. 342 |
| 167 | Seq. ID No. 345 | Seq. ID No. 346 |
| 171b | Seq. ID No. 353 | Seq. ID No. 354 |
| 175 | Seq. ID No. 361 | Seq. ID No. 362 |
| 176 | Seq. ID No. 363 | Seq. ID No. 364 |
| 177a | Seq. ID No. 365 | Seq. ID No. 366 |
| 180b | Seq. ID No. 375 | Seq. ID No. 376 |
| 184 | Seq. ID No. 379 | Seq. ID No. 380 |
| 186 | Seq. ID No. 383 | Seq. ID No. 384 |
| 188 | Seq. ID No. 389 | Seq. ID No. 390 |
| 189b | Seq. ID No. 391 | Seq. ID No. 392 |
| 192b | Seq. ID No. 397 | Seq. ID No. 398 |
| 195 | Seq. ID No. 405 | Seq. ID No. 406 |
| 197a | Seq. ID No. 409 | Seq. ID No. 410 |
| 198b | Seq. ID No. 415 | Seq. ID No. 416 |
| 203 | Seq. ID No. 423 | Seq. ID No. 424 |
| 204 | Seq. ID No. 425 | Seq. ID No. 426 |
| 205 | Seq. ID No. 427 | Seq. ID No. 428 |
| 206 | Seq. ID No. 429 | Seq. ID No. 430 |
| 208b | Seq. ID No. 435 | Seq. ID No. 436 |
| 210 | Seq. ID No. 437 | Seq. ID No. 438 |
| 211 | Seq. ID No. 439 | Seq. ID No. 440 |
| 213 | Seq. ID No. 441 | Seq. ID No. 442 |
| 215a | Seq. ID No. 443 | Seq. ID No. 444 |
| 218 | Seq. ID No. 445 | Seq. ID No. 446 |
| 219 | Seq. ID No. 447 | Seq. ID No. 448 |
| 220a | Seq. ID No. 449 | Seq. ID No. 450 |
| 226b | Seq. ID No. 461 | Seq. ID No. 462 |
| 227 | Seq. ID No. 463 | Seq. ID No. 464 |
| 228a | Seq. ID No. 465 | Seq. ID No. 466 |
| 249a | Seq. ID No. 513 | Seq. ID No. 514 |
| 262 | Seq. ID No. 543 | Seq. ID No. 544 |
| 265 | Seq. ID No. 551 | Seq. ID No. 552 |
| 267b | Seq. ID No. 553 | Seq. ID No. 554 |
| 269a | Seq. ID No. 559 | Seq. ID No. 560 |
| 284a | Seq. ID No. 595 | Seq. ID No. 596 |
| 286a | Seq. ID No. 601 | Seq. ID No. 602 |
| 311 | Seq. ID No. 637 | Seq. ID No. 638 |
| 315 | Seq. ID No. 649 | Seq. ID No. 650 |
| 318a | Seq. ID No. 657 | Seq. ID No. 658 |
| 321 | Seq. ID No. 669 | Seq. ID No. 670 |
| 324 | Seq. ID No. 675 | Seq. ID No. 676 |
| 329 | Seq. ID No. 683 | Seq. ID No. 684 |
| 351 | Seq. ID No. 753 | Seq. ID No. 754 |
| 429b | Seq. ID No. 929 | Seq. ID No. 930 |
| 450a | Seq. ID No. 969 | Seq. ID No. 970 |
| 473 | Seq. ID No. 1041 | Seq. ID No. 1042 |
| 474 | Seq. ID No. 1043 | Seq. ID No. 1044 |
| 498a | Seq. ID No. 1085 | Seq. ID No. 1086 |
| 499a | Seq. ID No. 1089 | Seq. ID No. 1090 |
| 500 | Seq. ID No. 1093 | Seq. ID No. 1094 |
| 502c | Seq. ID No. 1097 | Seq. ID No. 1098 |
| 504 | Seq. ID No. 1099 | Seq. ID No. 1100 |
| 505a | Seq. ID No. 1101 | Seq. ID No. 1102 |
| 543a | Seq. ID No. 1175 | Seq. ID No. 1176 |
| 551 | Seq. ID No. 1205 | Seq. ID No. 1206 |
| 570b | Seq. ID No. 1237 | Seq. ID No. 1238 |
| 687 | Seq. ID No. 1459 | Seq. ID No. 1460 |
| 709 | Seq. ID No. 1495 | Seq. ID No. 1496 |
| 710 | Seq. ID No. 1497 | Seq. ID No. 1498 |
| 715a | Seq. ID No. 1503 | Seq. ID No. 1504 |
| 722c | Seq. ID No. 1517 | Seq. ID No. 1518 |
| 726 | Seq. ID No. 1527 | Seq. ID No. 1528 |
| 728b | Seq. ID No. 1531 | Seq. ID No. 1532 |
| 730d | Seq. ID No. 1539 | Seq. ID No. 1540 |
| 732 | Seq. ID No. 1543 | Seq. ID No. 1544 |
| 738c | Seq. ID No. 1549 | Seq. ID No. 1550 |
| 747 | Seq. ID No. 1573 | Seq. ID No. 1574 |
| 792 | Seq. ID No. 1657 | Seq. ID No. 1658 |
| 829a | Seq. ID No. 1709 | Seq. ID No. 1710 |
| 859b | Seq. ID No. 1767 | Seq. ID No. 1768 |
| 866b | Seq. ID No. 1785 | Seq. ID No. 1786 |
| 886 | Seq. ID No. 1821 | Seq. ID No. 1822 |
| 938 | Seq. ID No. 1889 | Seq. ID No. 1890 |
| 954 | Seq. ID No. 1925 | Seq. ID No. 1926 |
| 977a | Seq. ID No. 1969 | Seq. ID No. 1970 |
| 992 | Seq. ID No. 2003 | Seq. ID No. 2004 |
| 1016c | Seq. ID No. 2047 | Seq. ID No. 2048 |
| 1033a | Seq. ID No. 2075 | Seq. ID No. 2076 |
| 1036b | Seq. ID No. 2089 | Seq. ID No. 2090 |
| 1037 | Seq. ID No. 2093 | Seq. ID No. 2094 |
| 1049a | Seq. ID No. 2117 | Seq. ID No. 2118 |
| 1058 | Seq. ID No. 2137 | Seq. ID No. 2138 |
| 1062 | Seq. ID No. 2143 | Seq. ID No. 2144 |
| 1094a | Seq. ID No. 2205 | Seq. ID No. 2206 |
| 1108b | Seq. ID No. 2235 | Seq. ID No. 2236 |
| 1131 | Seq. ID No. 2257 | Seq. ID No. 2258 |
| 1149 | Seq. ID No. 2301 | Seq. ID No. 2302 |
| 1171c | Seq. ID No. 2363 | Seq. ID No. 2364 |
| 1194 | Seq. ID No. 2423 | Seq. ID No. 2424 |
| 1205 | Seq. ID No. 2445 | Seq. ID No. 2446 |
| 1208 | Seq. ID No. 2451 | Seq. ID No. 2452 |
| 1221a | Seq. ID No. 2481 | Seq. ID No. 2482 |
| 1226 | Seq. ID No. 2495 | Seq. ID No. 2496 |
| 1230 | Seq. ID No. 2505 | Seq. ID No. 2506 |
| 1256 | Seq. ID No. 2577 | Seq. ID No. 2578 |
| 1258c | Seq. ID No. 2587 | Seq. ID No. 2588 |
| 1271 | Seq. ID No. 2613 | Seq. ID No. 2614 |
| 1281 | Seq. ID No. 2633 | Seq. ID No. 2634 |
| 1288 | Seq. ID No. 2651 | Seq. ID No. 2652 |
| 1297a | Seq. ID No. 2665 | Seq. ID No. 2666 |
| 1300 | Seq. ID No. 2669 | Seq. ID No. 2670 |
| 1305c | Seq. ID No. 2677 | Seq. ID No. 2678 |
| 1308e | Seq. ID No. 2687 | Seq. ID No. 2688 |
| 1315 | Seq. ID No. 2693 | Seq. ID No. 2694 |
| 1318 | Seq. ID No. 2699 | Seq. ID No. 2700 |
| 1321 | Seq. ID No. 2711 | Seq. ID No. 2712 |
| 1322b | Seq. ID No. 2715 | Seq. ID No. 2716 |
| 1349b | Seq. ID No. 2757 | Seq. ID No. 2758 |
| 1368 | Seq. ID No. 2791 | Seq. ID No. 2792 |
| 1371 | Seq. ID No. 2793 | Seq. ID No. 2794 |
| 1377 | Seq. ID No. 2803 | Seq. ID No. 2804 |
| 1408 | Seq. ID No. 2867 | Seq. ID No. 2868 |
| 1431 | Seq. ID No. 2919 | Seq. ID No. 2920 |
| 1437a | Seq. ID No. 2929 | Seq. ID No. 2930 |
| 1462b | Seq. ID No. 3005 | Seq. ID No. 3006 |
| 1466d | Seq. ID No. 3019 | Seq. ID No. 3020 |
| 1508 | Seq. ID No. 3125 | Seq. ID No. 3126 |
| 1512 | Seq. ID No. 3137 | Seq. ID No. 3138 |
| 1528 | Seq. ID No. 3161 | Seq. ID No. 3162 |
| 1537 | Seq. ID No. 3179 | Seq. ID No. 3180 |
| 1573 | Seq. ID No. 3251 | Seq. ID No. 3252 |
| 1578 | Seq. ID No. 3259 | Seq. ID No. 3260 |
| 1588 | Seq. ID No. 3285 | Seq. ID No. 3286 |
| 1600a | Seq. ID No. 3309 | Seq. ID No. 3310 |
| 1606a | Seq. ID No. 3317 | Seq. ID No. 3318 |
| 1619 | Seq. ID No. 3337 | Seq. ID No. 3338 |
| 1623b | Seq. ID No. 3345 | Seq. ID No. 3346 |
| 1626b | Seq. ID No. 3359 | Seq. ID No. 3360 |
| 1680 | Seq. ID No. 3475 | Seq. ID No. 3476 |
| 1694b | Seq. ID No. 3515 | Seq. ID No. 3516 |
| 1696 | Seq. ID No. 3521 | Seq. ID No. 3522 |
| 1759 | Seq. ID No. 3641 | Seq. ID No. 3642 |
| 1788c | Seq. ID No. 3695 | Seq. ID No. 3696 |
| 1816a | Seq. ID No. 3793 | Seq. ID No. 3794 |
| 1861b | Seq. ID No. 3901 | Seq. ID No. 3902 |
| 1875b | Seq. ID No. 3927 | Seq. ID No. 3928 |
| 1912 | Seq. ID No. 3987 | Seq. ID No. 3988 |
| 1918c | Seq. ID No. 4001 | Seq. ID No. 4002 |
| 1940 | Seq. ID No. 4057 | Seq. ID No. 4058 |
| 1951a | Seq. ID No. 4081 | Seq. ID No. 4082 |
| 1960c | Seq. ID No. 4111 | Seq. ID No. 4112 |
| 1967 | Seq. ID No. 4125 | Seq. ID No. 4126 |
| 1996 | Seq. ID No. 4193 | Seq. ID No. 4194 |
| 2000 | Seq. ID No. 4195 | Seq. ID No. 4196 |
| 2020 | Seq. ID No. 4237 | Seq. ID No. 4238 |
| 2024b | Seq. ID No. 4247 | Seq. ID No. 4248 |
| 2094 | Seq. ID No. 4383 | Seq. ID No. 4384 |
| 2097 | Seq. ID No. 4393 | Seq. ID No. 4394 |
| 2100 | Seq. ID No. 4397 | Seq. ID No. 4398 |
| 2104 | Seq. ID No. 4401 | Seq. ID No. 4402 |
| 2117 | Seq. ID No. 4435 | Seq. ID No. 4436 |
| 2125a | Seq. ID No. 4449 | Seq. ID No. 4450 |
| 2140b | Seq. ID No. 4471 | Seq. ID No. 4472 |
| 2149b | Seq. ID No. 4491 | Seq. ID No. 4492 |
| 2156c | Seq. ID No. 4511 | Seq. ID No. 4512 |
| 2160b | Seq. ID No. 4519 | Seq. ID No. 4520 |
| 2164 | Seq. ID No. 4531 | Seq. ID No. 4532 |
| 2174 | Seq. ID No. 4549 | Seq. ID No. 4550 |
| 2185 | Seq. ID No. 4577 | Seq. ID No. 4578 |
| 2186b | Seq. ID No. 4581 | Seq. ID No. 4582 |
| 2189b | Seq. ID No. 4585 | Seq. ID No. 4586 |
| 2203 | Seq. ID No. 4609 | Seq. ID No. 4610 |
| 2204 | Seq. ID No. 4611 | Seq. ID No. 4612 |
| 2206a | Seq. ID No. 4613 | Seq. ID No. 4614 |
| 2234a | Seq. ID No. 4675 | Seq. ID No. 4676 |
| 2238 | Seq. ID No. 4685 | Seq. ID No. 4686 |
| 2256 | Seq. ID No. 4719 | Seq. ID No. 4720 |
| 2260c | Seq. ID No. 4729 | Seq. ID No. 4730 |
| 2269 | Seq. ID No. 4743 | Seq. ID No. 4744 |
| 2275b | Seq. ID No. 4755 | Seq. ID No. 4756 |
| 2284 | Seq. ID No. 4781 | Seq. ID No. 4782 |
| 2286b | Seq. ID No, 4787 | Seq. ID No. 4788 |
| 2311 | Seq. ID No. 4837 | Seq. ID No. 4838 |
| 2312b | Seq. ID No. 4841 | Seq. ID No. 4842 |
| 2316 | Seq. ID No. 4849 | Seq. ID No. 4850 |
| 2321a | Seq. ID No. 4855 | Seq. ID No. 4856 |
| 2338a | Seq. ID No. 4889 | Seq. ID No. 4890 |
| 2344 | Seq. ID No. 4901 | Seq. ID No. 4902 |
| 2353 | Seq. ID No. 4915 | Seq. ID No. 4916 |
| 2354 | Seq. ID No. 4917 | Seq. ID No. 4918 |
| 2363b | Seq. ID No. 4931 | Seq. ID No. 4932 |
| 2365b | Seq. ID No. 4935 | Seq. ID No. 4936 |
| 2384a | Seq. ID No. 4977 | Seq. ID No. 4978 |
| 2409a | Seq. ID No. 5025 | Seq. ID No. 5026 |
| 2410 | Seq. ID No. 5029 | Seq. ID No. 5030 |
| 2449b | Seq. ID No. 5109 | Seq. ID No. 5110 |
| 2453 | Seq. ID No. 5115 | Seq. ID No. 5116 |
| 2455 | Seq. ID No. 5117 | Seq. ID No. 5118 |
| 2465a | Seq. ID No. 5155 | Seq. ID No. 5156 |
| 2477 | Seq. ID No. 5185 | Seq. ID No. 5186 |
| 2485 | Seq. ID No. 5201 | Seq. ID No. 5202 |
| 2489 | Seq. ID No. 5203 | Seq. ID No. 5204 |
| 2497b | Seq. ID No. 5219 | Seq. ID No. 5220 |
| 2516b | Seq. ID No. 5261 | Seq. ID No. 5262 |
| 2527a | Seq. ID No. 5289 | Seq. ID No. 5290 |
| 2528b | Seq. ID No. 5295 | Seq. ID No. 5296 |
| 2564b | Seq. ID No. 5377 | Seq. ID No. 5378 |
| 2568 | Seq. ID No. 5389 | Seq. ID No. 5390 |
| 2578 | Seq. ID No. 5399 | Seq. ID No. 5400 |
| 2585 | Seq. ID No. 5411 | Seq. ID No. 5412 |
| 2643b | Seq. ID No. 5537 | Seq. ID No. 5538 |
| 2680c | Seq. ID No. 5621 | Seq. ID No. 5622 |
| 2684a | Seq. ID No. 5627 | Seq. ID No. 5628 |
| 2695 | Seq. ID No. 5657 | Seq. ID No. 5658 |
| 2716a | Seq. ID No. 5675 | Seq. ID No. 5676 |
| 2732 | Seq. ID No. 5717 | Seq. ID No. 5718 |
| 2752 | Seq. ID No. 5777 | Seq. ID No. 5778 |
| 2785b | Seq. ID No. 5859 | Seq. ID No. 5860 |
| 2805b | Seq. ID No. 5901 | Seq. ID No. 5902 |
| 2812 | Seq. ID No. 5913 | Seq. ID No. 5914 |
| 2816 | Seq. ID No. 5919 | Seq. ID No. 5920 |
| 2818c | Seq. ID No. 5925 | Seq. ID No. 5926 |
| 2820 | Seq. ID No. 5933 | Seq. ID No. 5934 |
| 2886a | Seq. ID No. 6055 | Seq. ID No. 6056 |
| 2918 | Seq. ID No. 6129 | Seq. ID No. 6130 |
| 2921 | Seq. ID No. 6133 | Seq. ID No. 6134 |
| 2941c | Seq. ID No. 6187 | Seq. ID No. 6188 |
| 2943c | Seq. ID No. 6193 | Seq. ID No. 6194 |
| 2954 | Seq. ID No. 6219 | Seq. ID No. 6220 |
| 2971b | Seq. ID No. 6271 | Seq. ID No. 6272 |
| 2975 | Seq. ID No. 6277 | Seq. ID No. 6278 |
| 3017 | Seq. ID No. 6369 | Seq. ID No. 6370 |
| 3020 | Seq. ID No. 6375 | Seq. ID No. 6376 |
| 3022 | Seq. ID No. 6379 | Seq. ID No. 6380 |
| 3026b | Seq. ID No. 6389 | Seq. ID No. 6390 |
| 3118b | Seq. ID No. 6591 | Seq. ID No. 6592 |
| 3121 | Seq. ID No. 6605 | Seq. ID No. 6606 |

Listed in Table 10 are 22 ORFs predicted to be covalently bound to the peptidoglycan region via the ‘LPXTG’ motif. These ORFs were found using the HMM LPXTG motif finder and are classified as proteins that might be targeted by sortase. The Applicants developed a HMM using approximately 70 known prokaryotic polypeptides containing the LPXTG cell wall sorting signal. This HMM was used to predict cell wall polypeptides that are anchored to the peptidoglycan layer.

TABLE 10
LPXTG MOTIF

| ORF Number | DNA SEQ ID Number | Protein SEQ ID Number |
|---|---|---|
| 246a | Seq. ID No. 503 | Seq. ID No. 504 |
| 246b | Seq. ID No. 505 | Seq. ID No. 506 |
| 609a | Seq. ID No. 1315 | Seq. ID No. 1316 |
| 609b | Seq. ID No. 1317 | Seq. ID No. 1318 |
| 631a | Seq. ID No. 1363 | Seq. ID No. 1364 |
| 631b | Seq. ID No. 1365 | Seq. ID No. 1366 |
| 631c | Seq. ID No. 1367 | Seq. ID No. 1368 |
| 631d | Seq. ID No. 1369 | Seq. ID No. 1370 |
| 743a | Seq. ID No. 1559 | Seq. ID No. 1560 |
| 743b | Seq. ID No. 1561 | Seq. ID No. 1562 |
| 751 | Seq. ID No. 1581 | Seq. ID No. 1582 |
| 847a | Seq. ID No. 1739 | Seq. ID No. 1740 |
| 847b | Seq. ID No. 1741 | Seq. ID No. 1742 |
| 1381a | Seq. ID No. 2811 | Seq. ID No. 2812 |
| 1381b | Seq. ID No. 2813 | Seq. ID No. 2814 |
| 1747 | Seq. ID No. 3619 | Seq. ID No. 3620 |
| 2669a | Seq. ID No. 5587 | Seq. ID No. 5588 |
| 2669b | Seq. ID No. 5589 | Seq. ID No. 5590 |
| 2669c | Seq. ID No. 5591 | Seq. ID No. 5592 |
| 2757a | Seq. ID No. 5781 | Seq. ID No. 5782 |
| 2757b | Seq. ID No. 5783 | Seq. ID No. 5784 |
| 2906 | Seq. ID No. 6103 | Seq. ID No. 6104 |

Listed in Table 11 are 71 ORFs predicted to be lipoproteins based on the HMM Lipo program.

TABLE 11
LIPOPROTEIN

| ORF Number | DNA SEQ ID Number | Protein SEQ ID Number |
|---|---|---|
| 38a | Seq. ID No. 45 | Seq. ID No. 46 |
| 38b | Seq. ID No. 47 | Seq. ID No. 48 |
| 91a | Seq. ID No. 171 | Seq. ID No. 172 |
| 114 | Seq. ID No. 225 | Seq. ID No. 226 |
| 201a | Seq. ID No. 419 | Seq. ID No. 420 |
| 249a | Seq. ID No. 513 | Seq. ID No. 514 |
| 277c | Seq. ID No. 575 | Seq. ID No. 576 |
| 283a | Seq. ID No. 591 | Seq. ID No. 592 |
| 283b | Seq. ID No. 593 | Seq. ID No. 594 |
| 318a | Seq. ID No. 657 | Seq. ID No. 658 |
| 318b | Seq. ID No. 659 | Seq. ID No. 660 |
| 321 | Seq. ID No. 669 | Seq. ID No. 670 |
| 324 | Seq. ID No. 675 | Seq. ID No. 676 |
| 349b | Seq. ID No. 745 | Seq. ID No. 746 |
| 349c | Seq. ID No. 747 | Seq. ID No. 748 |
| 431 | Seq. ID No. 935 | Seq. ID No. 936 |
| 476 | Seq. ID No. 1045 | Seq. ID No. 1046 |
| 492 | Seq. ID No. 1075 | Seq. ID No. 1076 |
| 524b | Seq. ID No. 1137 | Seq. ID No. 1138 |
| 542 | Seq. ID No. 1173 | Seq. ID No. 1174 |
| 546b | Seq. ID No. 1191 | Seq. ID No. 1192 |
| 546c | Seq. ID No. 1193 | Seq. ID No. 1194 |
| 547a | Seq. ID No. 1195 | Seq. ID No. 1196 |
| 744 | Seq. ID No. 1563 | Seq. ID No. 1564 |
| 794b | Seq. ID No. 1661 | Seq. ID No. 1662 |
| 794c | Seq. ID No. 1663 | Seq. ID No. 1664 |
| 794d | Seq. ID No. 1665 | Seq. ID No. 1666 |
| 870 | Seq. ID No. 1791 | Seq. ID No. 1792 |
| 873c | Seq. ID No. 1797 | Seq. ID No. 1798 |
| 951a | Seq. ID No. 1917 | Seq. ID No. 1918 |
| 977a | Seq. ID No. 1969 | Seq. ID No. 1970 |
| 1047b | Seq. ID No. 2115 | Seq. ID No. 2116 |
| 1067 | Seq. ID No. 2159 | Seq. ID No. 2160 |
| 1197 | Seq. ID No. 2429 | Seq. ID No. 2430 |
| 1239 | Seq. ID No. 2527 | Seq. ID No. 2528 |
| 1318 | Seq. ID No. 2699 | Seq. ID No. 2700 |
| 1382 | Seq. ID No. 2815 | Seq. ID No. 2816 |
| 1411b | Seq. ID No. 2875 | Seq. ID No. 2876 |
| 1449a | Seq. ID No. 2975 | Seq. ID No. 2976 |
| 1449b | Seq. ID No. 2977 | Seq. ID No. 2978 |
| 1455b | Seq. ID No. 2991 | Seq. ID No. 2992 |
| 1475 | Seq. ID No. 3043 | Seq. ID No. 3044 |
| 1607b | Seq. ID No. 3323 | Seq. ID No. 3324 |
| 1619 | Seq. ID No. 3337 | Seq. ID No. 3338 |
| 1970b | Seq. ID No. 4133 | Seq. ID No. 4134 |
| 1973 | Seq. ID No. 4137 | Seq. ID No. 4138 |
| 2146 | Seq. ID No. 4481 | Seq. ID No. 4482 |
| 2249 | Seq. ID No. 4705 | Seq. ID No. 4706 |
| 2308 | Seq. ID No. 4827 | Seq. ID No. 4828 |
| 2329a | Seq. ID No. 4869 | Seq. ID No. 4870 |
| 2453 | Seq. ID No. 5115 | Seq. ID No. 5116 |
| 2564b | Seq. ID No. 5377 | Seq. ID No. 5378 |
| 2564c | Seq. ID No. 5379 | Seq. ID No. 5380 |
| 2623b | Seq. ID No. 5491 | Seq. ID No. 5492 |
| 2628 | Seq. ID No. 5509 | Seq. ID No. 5510 |
| 2629b | Seq. ID No. 5513 | Seq. ID No. 5514 |
| 2633d | Seq. ID No. 5525 | Seq. ID No. 5526 |
| 2637a | Seq. ID No. 5529 | Seq. ID No. 5530 |
| 2637b | Seq. ID No. 5531 | Seq. ID No. 5532 |
| 2799a | Seq. ID No. 5889 | Seq. ID No. 5890 |
| 2810c | Seq. ID No. 5909 | Seq. ID No. 5910 |
| 2869b | Seq. ID No. 6025 | Seq. ID No. 6026 |
| 2869c | Seq. ID No. 6027 | Seq. ID No. 6028 |
| 2900a | Seq. ID No. 6087 | Seq. ID No. 6088 |
| 2900b | Seq. ID No. 6089 | Seq. ID No. 6090 |
| 2993a | Seq. ID No. 6325 | Seq. ID No. 6326 |
| 2993b | Seq. ID No. 6327 | Seq. ID No. 6328 |
| 3037a | Seq. ID No. 6415 | Seq. ID No. 6416 |
| 3145b | Seq. ID No. 6637 | Seq. ID No. 6638 |
| 3145c | Seq. ID No. 6639 | Seq. ID No. 6640 |
| 3147c | Seq. ID No. 6645 | Seq. ID No. 6646 |

Listed in Table 12 are ORFs encoding polypeptides predicted to be noncovalently bound to the peptidoglycan layer based on the HMM program. Four ORFs were identified that were predicted to encode proteins that bound peptidoglycans noncovalently.

TABLE 12
PEPTIDOGLYCAN BINDING

| ORF Number | DNA SEQ ID Number | Protein SEQ ID Number |
|---|---|---|
| 1733 | Seq. ID No. 3589 | Seq. ID No. 3590 |
| 1912 | Seq. ID No. 3987 | Seq. ID No. 3988 |
| 2497b | Seq. ID No. 5219 | Seq. ID No. 5220 |
| 2549 | Seq. ID No. 5337 | Seq. ID No. 5338 |

Also contemplated in the present invention are other important characteristics of Alloiococcus otitidis ORFs. For example, the Alloiococcus otitidis ORFs were searched to identify any that encode polypeptides with RGD motifs. Proteins that contain the Arg-Gly-Asp (RGD) attachment motif, together with integrins that serve as their receptor, constitute a major recognition system for cell adhesion, and thus are putative Alloiococcus otitidis polypeptide antigens. RGD recognition is one mechanism used by microbes to gain entry into eukaryotic tissues (Stockbauer et al., 1999; Isberg and Nhieu, 1994). However, not all RGD-containing proteins mediate cell attachment. It has been shown that RGD-containing peptides with a proline at the carboxy end (RGDP) are inactive in cell attachment assays (Pierschbacher and Rouslahti, 1987) and were excluded from consideration.

Listed in Table 13 are 125 ORFs found to contain the peptide sequence ‘RGDX’ where X is not proline. These ORFs are predicted to be surface localized and may interact with eukaryotic extracellular proteins.

TABLE 13
RGD MOTIF

| ORF Number | DNA SEQ ID Number | Protein SEQ ID Number |
|---|---|---|
| 24 | Seq. ID No. 37 | Seq. ID No. 38 |
| 58 | Seq. ID No. 99 | Seq. ID No. 100 |
| 62a | Seq. ID No. 107 | Seq. ID No. 108 |
| 62b | Seq. ID No. 109 | Seq. ID No. 110 |
| 62c | Seq. ID No. 111 | Seq. ID No. 112 |
| 171a | Seq. ID No. 351 | Seq. ID No. 352 |
| 171b | Seq. ID No. 353 | Seq. ID No. 354 |
| 228a | Seq. ID No. 465 | Seq. ID No. 466 |
| 228b | Seq. ID No. 467 | Seq. ID No. 468 |
| 228c | Seq. ID No. 469 | Seq. ID No. 470 |
| 228d | Seq. ID No. 471 | Seq. ID No. 472 |
| 329 | Seq. ID No. 683 | Seq. ID No. 684 |
| 383a | Seq. ID No. 821 | Seq. ID No. 822 |
| 383b | Seq. ID No. 823 | Seq. ID No. 824 |
| 467a | Seq. ID No. 1021 | Seq. ID No. 1022 |
| 467b | Seq. ID No. 1023 | Seq. ID No. 1024 |
| 535 | Seq. ID No. 1157 | Seq. ID No. 1158 |
| 536 | Seq. ID No. 1159 | Seq. ID No. 1160 |
| 569 | Seq. ID No. 1233 | Seq. ID No. 1234 |
| 614a | Seq. ID No: 1333 | Seq. ID No. 1334 |
| 614b | Seq. ID No. 1335 | Seq. ID No. 1336 |
| 680 | Seq. ID No. 1449 | Seq. ID No. 1450 |
| 743a | Seq. ID No. 1559 | Seq. ID No. 1560 |
| 743b | Seq. ID No. 1561 | Seq. ID No. 1562 |
| 744 | Seq. ID No. 1563 | Seq. ID No. 1564 |
| 774 | Seq. ID No. 1625 | Seq. ID No. 1626 |
| 863 | Seq. ID No. 1777 | Seq. ID No. 1778 |
| 890a | Seq. ID No. 1827 | Seq. ID No. 1828 |
| 890b | Seq. ID No. 1829 | Seq. ID No. 1830 |
| 929b | Seq. ID No. 1871 | Seq. ID No. 1872 |
| 954 | Seq. ID No. 1925 | Seq. ID No. 1926 |
| 1057 | Seq. ID No. 2135 | Seq. ID No. 2136 |
| 1063 | Seq. ID No. 2145 | Seq. ID No. 2146 |
| 1077b | Seq. ID No. 2173 | Seq. ID No. 2174 |
| 1077c | Seq. ID No. 2175 | Seq. ID No. 2176 |
| 1077d | Seq. ID No. 2177 | Seq. ID No. 2178 |
| 1077e | Seq. ID No. 2179 | Seq. ID No. 2180 |
| 1132 | Seq. ID No. 2259 | Seq. ID No. 2260 |
| 1155a | Seq. ID No. 2317 | Seq. ID No. 2318 |
| 1155b | Seq. ID No. 2319 | Seq. ID No. 2320 |
| 1155c | Seq. ID No. 2321 | Seq. ID No. 2322 |
| 1155d | Seq. ID No. 2323 | Seq. ID No. 2324 |
| 1155e | Seq. ID No. 2325 | Seq. ID No. 2326 |
| 1155f | Seq. ID No. 2327 | Seq. ID No. 2328 |
| 1155g | Seq. ID No. 2329 | Seq. ID No. 2330 |
| 1184 | Seq. ID No. 2401 | Seq. ID No. 2402 |
| 1302 | Seq. ID No. 2671 | Seq. ID No. 2672 |
| 1405 | Seq. ID No. 2863 | Seq. ID No. 2864 |
| 1409 | Seq. ID No. 2869 | Seq. ID No. 2870 |
| 1431 | Seq. ID No. 2919 | Seq. ID No. 2920 |
| 1432 | Seq. ID No. 2921 | Seq. ID No. 2922 |
| 1456 | Seq. ID No. 2993 | Seq. ID No. 2994 |
| 1489 | Seq. ID No. 3087 | Seq. ID No. 3088 |
| 1512 | Seq. ID No. 3137 | Seq. ID No. 3138 |
| 1587a | Seq. ID No. 3279 | Seq. ID No. 3280 |
| 1587b | Seq. ID No. 3281 | Seq. ID No. 3282 |
| 1587c | Seq. ID No. 3283 | Seq. ID No. 3284 |
| 1616a | Seq. ID No. 3333 | Seq. ID No. 3334 |
| 1616b | Seq. ID No. 3335 | Seq. ID No. 3336 |
| 1689a | Seq. ID No. 3497 | Seq. ID No. 3498 |
| 1689b | Seq. ID No. 3499 | Seq. ID No. 3500 |
| 1689c | Seq. ID No. 3501 | Seq. ID No. 3502 |
| 1700b | Seq. ID No. 3533 | Seq. ID No. 3534 |
| 1775a | Seq. ID No. 3671 | Seq. ID No. 3672 |
| 1775b | Seq. ID No. 3673 | Seq. ID No. 3674 |
| 1775c | Seq. ID No. 3675 | Seq. ID No. 3676 |
| 1838a | Seq. ID No. 3837 | Seq. ID No. 3838 |
| 1838b | Seq. ID No. 3839 | Seq. ID No. 3840 |
| 1838c | Seq. ID No. 3841 | Seq. ID No. 3842 |
| 1838d | Seq. ID No. 3843 | Seq. ID No. 3844 |
| 1875c | Seq. ID No. 3929 | Seq. ID No. 3930 |
| 1895 | Seq. ID No. 3961 | Seq. ID No. 3962 |
| 1934 | Seq. ID No. 4037 | Seq. ID No. 4038 |
| 1996 | Seq. ID No. 4193 | Seq. ID No. 4194 |
| 2021 | Seq. ID No. 4239 | Seq. ID No. 4240 |
| 2065a | Seq. ID No. 4327 | Seq. ID No. 4328 |
| 2065b | Seq. ID No. 4329 | Seq. ID No. 4330 |
| 2068 | Seq. ID No. 4333 | Seq. ID No. 4334 |
| 2097 | Seq. ID No. 4393 | Seq. ID No. 4394 |
| 2138a | Seq. ID No. 4463 | Seq. ID No. 4464 |
| 2138b | Seq. ID No. 4465 | Seq. ID No. 4466 |
| 2138c | Seq. ID No. 4467 | Seq. ID No. 4468 |
| 2196 | Seq. ID No. 4597 | Seq. ID No. 4598 |
| 2215a | Seq. ID No. 4629 | Seq. ID No. 4630 |
| 2215b | Seq. ID No. 4631 | Seq. ID No. 4632 |
| 2234a | Seq. ID No. 4675 | Seq. ID No. 4676 |
| 2234b | Seq. ID No. 4677 | Seq. ID No. 4678 |
| 2234c | Seq. ID No. 4679 | Seq. ID No. 4680 |
| 2240a | Seq. ID No. 4689 | Seq. ID No. 4690 |
| 2240b | Seq. ID No. 4691 | Seq. ID No. 4692 |
| 2240c | Seq. ID No. 4693 | Seq. ID No. 4694 |
| 2284 | Seq. ID No. 4781 | Seq. ID No. 4782 |
| 2292a | Seq. ID No. 4799 | Seq. ID No. 4800 |
| 2292b | Seq. ID No. 4801 | Seq. ID No. 4802 |
| 2292c | Seq. ID No. 4803 | Seq. ID No. 4804 |
| 2322 | Seq. ID No. 4861 | Seq. ID No. 4862 |
| 2401 | Seq. ID No. 5009 | Seq. ID No. 5010 |
| 2461a | Seq. ID No. 5143 | Seq. ID No. 5144 |
| 2461b | Seq. ID No. 5145 | Seq. ID No. 5146 |
| 2475a | Seq. ID No. 5179 | Seq. ID No. 5180 |
| 2475b | Seq. ID No. 5181 | Seq. ID No. 5182 |
| 2475c | Seq. ID No. 5183 | Seq. ID No. 5184 |
| 2513a | Seq. ID No. 5249 | Seq. ID No. 5250 |
| 2513b | Seq. ID No. 5251 | Seq. ID No. 5252 |
| 2513c | Seq. ID No. 5253 | Seq. ID No. 5254 |
| 2516a | Seq. ID No. 5259 | Seq. ID No. 5260 |
| 2516b | Seq. ID No. 5261 | Seq. ID No. 5262 |
| 2528a | Seq. ID No. 5293 | Seq. ID No. 5294 |
| 2528b | Seq. ID No. 5295 | Seq. ID No. 5296 |
| 2528c | Seq. ID No. 5297 | Seq. ID No. 5298 |
| 2528d | Seq. ID No. 5299 | Seq. ID No. 5300 |
| 2535a | Seq. ID No. 5307 | Seq. ID No. 5308 |
| 2535b | Seq. ID No. 5309 | Seq. ID No. 5310 |
| 2585 | Seq. ID No. 5411 | Seq. ID No. 5412 |
| 2643a | Seq. ID No. 5535 | Seq. ID No. 5536 |
| 2643b | Seq. ID No. 5537 | Seq. ID No. 5538 |
| 2744 | Seq. ID No. 5745 | Seq. ID No. 5746 |
| 2768a | Seq. ID No. 5821 | Seq. ID No. 5822 |
| 2768b | Seq. ID No. 5823 | Seq. ID No. 5824 |
| 2768c | Seq. ID No. 5825 | Seq. ID No. 5826 |
| 2871 | Seq. ID No. 6029 | Seq. ID No. 6030 |
| 2934 | Seq. ID No. 6171 | Seq. ID No. 6172 |
| 2988a | Seq. ID No. 6307 | Seq. ID No. 6308 |
| 2988b | Seq. ID No. 6309 | Seq. ID No. 6310 |
| 2988c | Seq. ID No. 6311 | Seq. ID No. 6312 |

Listed in Table 14 are the 39 ORFs whose BlastP results predict for possible capsule biosynthesis and transport. Keywords used in the search are: polysaccharide, saccharide, rhamnos, dexB, aliA, cap[A-Z], cps[A-Z], wze[A-Z], wzh[A-Z], teichu, tuaG, manB, pyrophosphorylase, glucose, eps[ABE], glucosyl, xylos, encapsula, pgs[ABC], exopolysacch, galactos, epimerase, lytR, lytABC, lysR, Transferase, GalNAc, GlcNAc, Spore, LPS, Peptidoglycan, Glutamate, Mannos, Oligosaccharide, Outer core, Arabinose, Teichoic, Teicho, Lipoteichoic, Glycerol, spo[A-Z], capsule, capsular.

TABLE 14
CAPSULE RELATED PROTEINS

| ORF | DNA SEQ ID | Protein SEQ ID | | | Blast ‘e’ |
|---|---|---|---|---|---|
| Number | Number | Number | Keyword | Description | Value |
| 39a | Seq. ID No. 49 | Seq. ID No. 50 | capC | capsule biosynthesis | 6.00E−19 |
| | | | | protein CapC, (pXO2- | |
| | | | | 57), Baci . . . | |
| 39b | Seq. ID No. 51 | Seq. ID No. 52 | capC | capsule biosynthesis | 7.00E−19 |
| | | | | protein CapC, (pXO2- | |
| | | | | 57), Baci . . . | |
| 40 | Seq. ID No. 53 | Seq. ID No. 54 | capB | CapB, Bacillus subtilis | 9.00E−95 |
| | | | | Bacteria; Firmicutes; | |
| | | | | Baci . . . | |
| 41c | Seq. ID No. 59 | Seq. ID No. 60 | capA | PgsA, Bacillus subtilis | 9.00E−95 |
| | | | | Bacteria; Firmicutes; | |
| | | | | Baci . . . | |
| 102 | Seq. ID No. 195 | Seq. ID No. 196 | saccharide | Bacillus cereus | 1.00E−168 |
| | | | | Bacteria; Firmicutes; | |
| | | | | Bacillus/Clo . . . | |
| 330c | Seq. ID No. 689 | Seq. ID No. 690 | transferase | Glycosyltransferase, | 5.00E−79 |
| | | | | Clostridium | |
| | | | | acetobutylicum Ba . . . | |
| 337 | Seq. ID No. 703 | Seq. ID No. 704 | transferase | glycosyltransferase, | 2.00E−58 |
| | | | | Lactococcus lactis | |
| | | | | subsp. lac . . . | |
| 427c | Seq. ID No. 925 | Seq. ID No. 926 | capsular | unknown, | 2.00E−46 |
| | | | | Staphylococcus aureus | |
| | | | | Bacteria; Firmicute . . . | |
| 760 | Seq. ID No. | Seq. ID No. 1598 | dexB | maturase-related | 6.00E−78 |
| | 1597 | | | protein, Streptococcus | |
| | | | | pneumoniae . . . | |
| 762b | Seq. ID No. | Seq. ID No. 1602 | cpsL | transposase, | 1.00E−46 |
| | 1601 | | | Streptococcus | |
| | | | | pneumoniae Bacteria; | |
| | | | | Fi . . . | |
| 763b | Seq. ID No. | Seq. ID No. 1608 | capsular | transposase, | 5.00E−44 |
| | 1607 | | | Staphylococcus aureus | |
| | | | | Bacteria; Firmi . . . | |
| 766 | Seq. ID No. | Seq. ID No. 1612 | cpsL | transposase, | 6.00E−44 |
| | 1611 | | | Streptococcus | |
| | | | | pneumoniae Bacteria; | |
| | | | | Fi . . . | |
| 767 | Seq. ID No. | Seq. ID No. 1614 | capsular | transposase, | 4.00E−16 |
| | 1613 | | | Staphylococcus aureus | |
| | | | | Bacteria; Firmi . . . | |
| 830 | Seq. ID No. | Seq. ID No. 1714 | cpsA | Thermostable | 4.00E−14 |
| | 1713 | | | carboxypeptidase | |
| | | | | (cpsA-2), Sulfolobus . . . | |
| 1004a | Seq. ID No. | Seq ID No. 2030 | EpsE | unknown, Lactobacillus | 1.00E−168 |
| | 2029 | | | rhamnosus Bacteria; | |
| | | | | Firmicu . . . | |
| 1135b | Seq. ID No. | Seq. ID No. 2264 | capA | Bacillus anthracis | 3.00E−25 |
| | 2263 | | | Bacteria; Firmicutes; | |
| | | | | Bacillus/ . . . | |
| 1138 | Seq. ID No. | Seq. ID No. 2270 | saccharide | unknown, | 9.00E−60 |
| | 2269 | | | Staphylococcus aureus | |
| | | | | Bacteria; Firmicute . . . | |
| 1177 | Seq. ID No. | Seq. ID No. 2374 | capsular | capsular | 9.00E−60 |
| | 2373 | | | polysaccharide | |
| | | | | biosynthesi . . . | |
| | | | | U67548 . . . | |
| 1200 | Seq. ID No. | Seq. ID No. 2438 | xylos | acetylxylosidase, | 9.00E−36 |
| | 2437 | | | Caldicellulosiruptor | |
| | | | | saccharolyt . . . | |
| 1288 | Seq. ID No. | Seq. ID No. 2652 | lytR | membrane bound | 3.00E−42 |
| | 2651 | | | protein, Bacillus subtilis | |
| | | | | Bacteria . . . | |
| 1355 | Seq. ID No. | Seq. ID No. 2768 | manB | probable | 1.00E−136 |
| | 2767 | | | phosphomannomutase, | |
| | | | | Clostridium | |
| | | | | perfringe . . . | |
| 1426b | Seq. ID No. | Seq. ID No. 2908 | polysaccharide | Orfde2, Enterococcus | 1.00E−103 |
| | 2907 | | | faecalis Bacteria; | |
| | | | | Firmicutes . . . | |
| 1646e | Seq. ID No. | Seq. ID No. 3398 | polysaccharide | putative polysaccharide | 3.00E−24 |
| | 3397 | | | biosynthesis protein, | |
| | | | | Stre . . . | |
| 1646f | Seq. ID No. | Seq. ID No. 3400 | polysaccharide | putative polysaccharide | 3.00E−78 |
| | 3399 | | | biosynthesis protein, | |
| | | | | Stre . . . | |
| 1682b | Seq. ID No. | Seq. ID No. 3480 | polysaccharide | methionine | 2.00E−68 |
| | 3479 | | | aminopeptidase A, | |
| | | | | Enterococcus | |
| | | | | faecalis . . . | |
| 1685 | Seq. ID No. | Seq. ID No. 3488 | glucose | UDP-glucose 4- | 1.00E−111 |
| | 3487 | | | epimerase, | |
| | | | | Streptococcus | |
| | | | | pneumoniae . . . | |
| 1688a | Seq. ID No. | Seq. ID No. 3492 | polysaccharide | Orfde3, Enterococcus | 3.00E−78 |
| | 3491 | | | faecalis Bacteria; | |
| | | | | Firmicutes . . . | |
| 1688b | Seq. ID No. | Seq. ID No. 3494 | polysaccharide | Orfde3, Enterococcus | 9.00E−34 |
| | 3493 | | | faecalis Bacteria; | |
| | | | | Firmicutes . . . | |
| 1688c | Seq. ID No. | Seq. ID No. 3496 | transferase | RgpG, Lactobacillus | 4.00E−99 |
| | 3495 | | | delbrueckii Bacteria; | |
| | | | | Firmicut . . . | |
| 1689a | Seq. ID No. | Seq. ID No. 3498 | tuaG | teichuronic acid | 9.00E−53 |
| | 3497 | | | biosynthesis, Bacillus | |
| | | | | halodurans . . . | |
| 1094d | Seq. ID No. | Seq. ID No. 3520 | lytR | attenuator for lytABC | 6.00E−69 |
| | 3519 | | | and lytR expression, | |
| | | | | Bacillu . . . | |
| 1696 | Seq. ID No. | Seq. ID No. 3522 | glucose | putative UDP-glucose | 1.00E−157 |
| | 3521 | | | dehydrogenase, | |
| | | | | Escherichia co . . . | |
| 1699b | Seq. ID No. | Seq. ID No. 3530 | teichu | unknown, Bacillus | 8.00E−30 |
| | 3529 | | | subtilis Bacteria; | |
| | | | | Firmicutes; G . . . | |
| 1701a | Seq. ID No. | Seq. ID No. 3536 | mannos | alpha-D-mannose- | 1.00E−84 |
| | 3535 | | | alpha(1-6)phospha . . . | |
| | | | | AP001516 . . . | |
| 1710b | Seq. ID No. | Seq. ID No. 3562 | LPS | LPS biosynthesis | 4.00E−12 |
| | 3561 | | | protein RfaE, Ps . . . | |
| | | | | AE004912 . . . | |
| 1855 | Seq. ID No. | Seq. ID No. 3884 | capsule | Regulatory function on | 5.00E−38 |
| | 3883 | | | capsule ex . . . | |
| | | | | AE008458 . . . | |
| 1857c | Seq. ID No. | Seq. ID No. 3892 | polysaccharide | Staphylococcus aureus | 1.00E−13 |
| | 3891 | | | subsp. aureus N315 | |
| | | | | Bacteria; . . . | |
| 1902a | Seq. ID No. | Seq. ID No. 3982 | capsular | DNA topoisomerase IV | 7.00E−15 |
| | 3981 | | | subunit B, | |
| | | | | Streptococcus agal . . . | |
| 1951d | Seq. ID No. | Seq. ID No. 4088 | saccharide | Phospholipid- | 7.00E−84 |
| | 4087 | | | lipopolysaccharide A . . . | |
| | | | | AE010571 . . . | |

Three potential capsule loci have been identified based on BlastP analysis listed in Table 14: poly-gamma-glutamate from base 20186 to 24645, sugar transport from base 159934 to 172757, and teichuronic from base 886658 to 930066. Table 15 lists the ORFs found within these regions.

TABLE 15
CAPSULE LOCI

| ORF | DNA SEQ ID | Protein SEQ | |
|---|---|---|---|
| Number | Number | ID Number | Locus |
| 38a | Seq. ID No. 45 | Seq. ID No. 46 | Poly-gamma-glutamate |
| 38b | Seq. ID No. 47 | Seq. ID No. 48 | Poly-gamma-glutamate |
| 39b | Seq. ID No. 51 | Seq. ID No. 52 | Poly-gamma-glutamate |
| 40 | Seq. ID No. 53 | Seq. ID No. 54 | Poly-gamma-glutamate |
| 41a | Seq. ID No. 55 | Seq. ID No. 56 | Poly-gamma-glutamate |
| 41b | Seq. ID No. 57 | Seq. ID No. 58 | Poly-gamma-glutamate |
| 41c | Seq. ID No. 59 | Seq. ID No. 60 | Poly-gamma-glutamate |
| 329 | Seq. ID No. 683 | Seq. ID No. 684 | sugar transport |
| 330a | Seq. ID No. 685 | Seq. ID No. 686 | sugar transport |
| 330b | Seq. ID No. 687 | Seq. ID No. 688 | sugar transport |
| 330c | Seq. ID No. 689 | Seq. ID No. 690 | sugar transport |
| 331 | Seq. ID No. 691 | Seq. ID No. 670 | sugar transport |
| 332 | Seq. ID No. 693 | Seq. ID No. 694 | sugar transport |
| 333 | Seq. ID No. 695 | Seq. ID No. 696 | sugar transport |
| 334 | Seq. ID No. 687 | Seq. ID No. 698 | sugar transport |
| 335a | Seq. ID No. 699 | Seq. ID No. 670 | sugar transport |
| 335b | Seq. ID No. 701 | Seq. ID No. 702 | sugar transport |
| 337 | Seq. ID No. 703 | Seq. ID No. 704 | sugar transport |
| 338a | Seq. ID No. 705 | Seq. ID No. 706 | sugar transport |
| 338b | Seq. ID No. 707 | Seq. ID No. 708 | sugar transport |
| 338c | Seq. ID No. 709 | Seq. ID No. 710 | sugar transport |
| 341a | Seq. ID No. 711 | Seq. ID No. 712 | sugar transport |
| 341b | Seq. ID No. 713 | Seq. ID No. 714 | sugar transport |
| 342a | Seq. ID No. 715 | Seq. ID No. 716 | sugar transport |
| 342b | Seq. ID No. 717 | Seq. ID No. 718 | sugar transport |
| 342c | Seq. ID No. 719 | Seq. ID No. 720 | sugar transport |
| 342d | Seq. ID No. 721 | Seq. ID No. 722 | sugar transport |
| 342e | Seq. ID No. 723 | Seq. ID No. 724 | sugar transport |
| 342f | Seq. ID No. 725 | Seq. ID No. 726 | sugar transport |
| 342g | Seq. ID No. 727 | Seq. ID No. 728 | sugar transport |
| 342h | Seq. ID No. 729 | Seq. ID No. 730 | sugar transport |
| 344a | Seq. ID No. 731 | Seq. ID No. 732 | sugar transport |
| 344b | Seq. ID No. 733 | Seq. ID No. 734 | sugar transport |
| 345 | Seq. ID No. 735 | Seq. ID No. 736 | sugar transport |
| 346a | Seq. ID No. 737 | Seq. ID No. 738 | sugar transport |
| 346b | Seq. ID No. 739 | Seq. ID No. 740 | sugar transport |
| 347 | Seq. ID No. 741 | Seq. ID No. 742 | sugar transport |
| 349a | Seq. ID No. 743 | Seq. ID No. 744 | sugar transport |
| 349b | Seq. ID No. 745 | Seq. ID No. 746 | sugar transport |
| 349c | Seq. ID No. 747 | Seq. ID No. 748 | sugar transport |
| 350a | Seq. ID No. 749 | Seq. ID No. 750 | sugar transport |
| 350b | Seq. ID No. 751 | Seq. ID No. 752 | sugar transport |
| 351 | Seq. ID No. 753 | Seq. ID No. 754 | sugar transport |
| 1646a | Seq. ID No. 3389 | Seq. ID No. 3390 | Teichuronic |
| 1646b | Seq. ID No. 3391 | Seq. ID No. 3392 | Teichuronic |
| 1646c | Seq. ID No. 3393 | Seq. ID No. 3394 | Teichuronic |
| 1646d | Seq. ID No. 3395 | Seq. ID No. 3396 | Teichuronic |
| 1646e | Seq. ID No. 3397 | Seq. ID No. 3398 | Teichuronic |
| 1646f | Seq. ID No. 3399 | Seq. ID No. 3400 | Teichuronic |
| 1647 | Seq. ID No. 3401 | Seq. ID No. 3402 | Teichuronic |
| 1648b | Seq. ID No. 3403 | Seq. ID No. 3404 | Teichuronic |
| 1649a | Seq. ID No. 3405 | Seq. ID No. 3406 | Teichuronic |
| 1649b | Seq. ID No. 3407 | Seq. ID No. 3408 | Teichuronic |
| 1649c | Seq. ID No. 3409 | Seq. ID No. 3410 | Teichuronic |
| 1652a | Seq. ID No. 3411 | Seq. ID No. 3412 | Teichuronic |
| 1652b | Seq. ID No. 3413 | Seq. ID No. 3414 | Teichuronic |
| 1654 | Seq. ID No. 3415 | Seq. ID No. 3416 | Teichuronic |
| 1656b | Seq. ID No. 3417 | Seq. ID No. 3418 | Teichuronic |
| 1657 | Seq. ID No. 3419 | Seq. ID No. 3420 | Teichuronic |
| 1659a | Seq. ID No. 3421 | Seq. ID No. 3422 | Teichuronic |
| 1659b | Seq. ID No. 3423 | Seq. ID No. 3424 | Teichuronic |
| 1659c | Seq. ID No. 3425 | Seq. ID No. 3426 | Teichuronic |
| 1660b | Seq. ID No. 3427 | Seq. ID No. 3428 | Teichuronic |
| 1662a | Seq. ID No. 3429 | Seq. ID No. 3430 | Teichuronic |
| 1662b | Seq. ID No. 3431 | Seq. ID No. 3432 | Teichuronic |
| 1663 | Seq. ID No. 3433 | Seq. ID No. 3434 | Teichuronic |
| 1664 | Seq. ID No. 3435 | Seq. ID No. 3436 | Teichuronic |
| 1665a | Seq. ID No. 3437 | Seq. ID No. 3438 | Teichuronic |
| 1668 | Seq. ID No. 3439 | Seq. ID No. 3440 | Teichuronic |
| 1669 | Seq. ID No. 3441 | Seq. ID No. 3442 | Teichuronic |
| 1670a | Seq. ID No. 3443 | Seq. ID No. 3444 | Teichuronic |
| 1670b | Seq. ID No. 3445 | Seq. ID No. 3446 | Teichuronic |
| 1670c | Seq. ID No. 3447 | Seq. ID No. 3448 | Teichuronic |
| 1673a | Seq. ID No. 3449 | Seq. ID No. 3450 | Teichuronic |
| 1673b | Seq. ID No. 3451 | Seq. ID No. 3452 | Teichuronic |
| 1673c | Seq. ID No. 3453 | Seq. ID No. 3454 | Teichuronic |
| 1673d | Seq. ID No. 3455 | Seq. ID No. 3456 | Teichuronic |
| 1673e | Seq. ID No. 3457 | Seq. ID No. 3458 | Teichuronic |
| 1673f | Seq. ID No. 3459 | Seq. ID No. 3460 | Teichuronic |
| 1674 | Seq. ID No. 3461 | Seq. ID No. 3462 | Teichuronic |
| 1675a | Seq. ID No. 3463 | Seq. ID No. 3464 | Teichuronic |
| 1675b | Seq. ID No. 3465 | Seq. ID No. 3466 | Teichuronic |
| 1675c | Seq. ID No. 3467 | Seq. ID No. 3468 | Teichuronic |
| 1676 | Seq. ID No. 3469 | Seq. ID No. 3470 | Teichuronic |
| 1677 | Seq. ID No. 3471 | Seq. ID No. 3472 | Teichuronic |
| 1679 | Seq. ID No. 3473 | Seq. ID No. 3474 | Teichuronic |
| 1680 | Seq. ID No. 3475 | Seq. ID No. 3476 | Teichuronic |
| 1682a | Seq. ID No. 3477 | Seq. ID No. 3478 | Teichuronic |
| 1682b | Seq. ID No. 3479 | Seq. ID No. 3480 | Teichuronic |
| 1683a | Seq. ID No. 3481 | Seq. ID No. 3482 | Teichuronic |
| 1683b | Seq. ID No. 3483 | Seq. ID No. 3484 | Teichuronic |
| 1684 | Seq. ID No. 3485 | Seq. ID No. 3486 | Teichuronic |
| 1685 | Seq. ID No. 3487 | Seq. ID No. 3488 | Teichuronic |
| 1687 | Seq. ID No. 3489 | Seq. ID No. 3490 | Teichuronic |
| 1688a | Seq. ID No. 3491 | Seq. ID No. 3492 | Teichuronic |
| 1688b | Seq. ID No. 3493 | Seq. ID No. 3494 | Teichuronic |
| 1688c | Seq. ID No. 3495 | Seq. ID No. 3496 | Teichuronic |
| 1689a | Seq. ID No. 3497 | Seq. ID No. 3498 | Teichuronic |
| 1689b | Seq. ID No. 3499 | Seq. ID No. 3500 | Teichuronic |
| 1689c | Seq. ID No. 3501 | Seq. ID No. 3502 | Teichuronic |
| 1690a | Seq. ID No. 3503 | Seq. ID No. 3504 | Teichuronic |
| 1690b | Seq. ID No. 3505 | Seq. ID No. 3506 | Teichuronic |
| 1691 | Seq. ID No. 3507 | Seq. ID No. 3508 | Teichuronic |
| 1692 | Seq. ID No. 3509 | Seq. ID No. 3510 | Teichuronic |
| 1693 | Seq. ID No. 3511 | Seq. ID No. 3512 | Teichuronic |
| 1694a | Seq. ID No. 3513 | Seq. ID No. 3514 | Teichuronic |
| 1694b | Seq. ID No. 3515 | Seq. ID No. 3516 | Teichuronic |
| 1694c | Seq. ID No. 3517 | Seq. ID No. 3518 | Teichuronic |
| 1694d | Seq. ID No. 3519 | Seq. ID No. 3520 | Teichuronic |
| 1696 | Seq. ID No. 3521 | Seq. ID No. 3522 | Teichuronic |
| 1697a | Seq. ID No. 3523 | Seq. ID No. 3524 | Teichuronic |
| 1697b | Seq. ID No. 3525 | Seq. ID No. 3526 | Teichuronic |
| 1699a | Seq. ID No. 3527 | Seq. ID No. 3528 | Teichuronic |
| 1699b | Seq. ID No. 3529 | Seq. ID No. 3530 | Teichuronic |
| 1700a | Seq. ID No. 3531 | Seq. ID No. 3532 | Teichuronic |
| 1700b | Seq. ID No. 3533 | Seq. ID No. 3534 | Teichuronic |
| 1701a | Seq. ID No. 3535 | Seq. ID No. 3536 | Teichuronic |
| 1701b | Seq. ID No. 3537 | Seq. ID No. 3538 | Teichuronic |
| 1703b | Seq. ID No. 3539 | Seq. ID No. 3540 | Teichuronic |
| 1704a | Seq. ID No. 3541 | Seq. ID No. 3542 | Teichuronic |
| 1704b | Seq. ID No. 3543 | Seq. ID No. 3544 | Teichuronic |
| 1705a | Seq. ID No. 3545 | Seq. ID No. 3546 | Teichuronic |
| 1705b | Seq. ID No. 3547 | Seq. ID No. 3548 | Teichuronic |
| 1706 | Seq. ID No. 3549 | Seq. ID No. 3550 | Teichuronic |
| 1707a | Seq. ID No. 3551 | Seq. ID No. 3552 | Teichuronic |
| 1707b | Seq. ID No. 3553 | Seq. ID No. 3554 | Teichuronic |
| 1708 | Seq. ID No. 3555 | Seq. ID No. 3556 | Teichuronic |
| 1709 | Seq. ID No. 3557 | Seq. ID No. 3558 | Teichuronic |
| 1710a | Seq. ID No. 3559 | Seq. ID No. 3560 | Teichuronic |
| 1710b | Seq. ID No. 3561 | Seq. ID No. 3562 | Teichuronic |

Listed in Table 16 are 14 ORFs determined by keyword search of the BlastP associated with sporulation.

TABLE 16
SPORULATION RELATED PROTEINS

| ORF Number | DNA SEQ ID Number | Protein SEQ ID Number |
|---|---|---|
| 524b | Seq. ID No. 1137 | Seq. ID No. 1138 |
| 683b | Seq. ID No. 1455 | Seq. ID No. 1456 |
| 1058 | Seq. ID No. 2137 | Seq. ID No. 2138 |
| 1060b | Seq. ID No. 2141 | Seq. ID No. 2142 |
| 1070b | Seq. ID No. 2165 | Seq. ID No. 2166 |
| 1077e | Seq. ID No. 2179 | Seq. ID No. 2180 |
| 1149 | Seq. ID No. 2301 | Seq. ID No. 2302 |
| 1348 | Seq. ID No. 2753 | Seq. ID No. 2754 |
| 2011c | Seq. ID No. 4211 | Seq. ID No. 4212 |
| 2024d | Seq. ID No. 4251 | Seq. ID No. 4252 |
| 2065a | Seq. ID No. 4327 | Seq. ID No. 4328 |
| 2181a | Seq. ID No. 4569 | Seq. ID No. 4570 |
| 2256 | Seq. ID No. 4719 | Seq. ID No. 4720 |
| 2260c | Seq. ID No. 4729 | Seq. ID No. 4730 |

Listed in Table 17 are the 913 start to stop ORFs which are unique to Alloiococcus otitidis, or 653 unique Stop-Stop ORFs identified as having a BlastP ‘E Value’ of >e⁻¹⁰ (not identified separately in Table 17).

TABLE 17
UNIQUE ORFs

| ORF Number | DNA SEQ ID Number | Protein SEQ ID Number |
|---|---|---|
| 4a | Seq. ID No. 7 | Seq. ID No. 8 |
| 11 | Seq. ID No. 19 | Seq. ID No. 20 |
| 13a | Seq. ID No. 21 | Seq. ID No. 22 |
| 16 | Seq. ID No. 23 | Seq. ID No. 24 |
| 21b | Seq. ID No. 31 | Seq. ID No. 32 |
| 32 | Seq. ID No. 39 | Seq. ID No. 40 |
| 44a | Seq. ID No. 65 | Seq. ID No. 66 |
| 44b | Seq. ID No. 67 | Seq. ID No. 68 |
| 45 | Seq. ID No. 69 | Seq. ID No. 70 |
| 55 | Seq. ID No. 91 | Seq. ID No. 92 |
| 58 | Seq. ID No. 99 | Seq. ID No. 100 |
| 61 | Seq. ID No. 105 | Seq. ID No. 106 |
| 63a | Seq. ID No. 113 | Seq. ID No. 114 |
| 63b | Seq. ID No. 115 | Seq. ID No. 116 |
| 66a | Seq. ID No. 123 | Seq. ID No. 124 |
| 67 | Seq. ID No. 125 | Seq. ID No. 126 |
| 69 | Seq. ID No. 127 | Seq. ID No. 128 |
| 73a | Seq. ID No. 131 | Seq. ID No. 132 |
| 73b | Seq. ID No. 133 | Seq. ID No. 134 |
| 79 | Seq. ID No. 139 | Seq. ID No. 140 |
| 83 | Seq. ID No. 151 | Seq. ID No. 152 |
| 86 | Seq. ID No. 159 | Seq. ID No. 160 |
| 87a | Seq. ID No. 161 | Seq. ID No. 162 |
| 87b | Seq. ID No. 163 | Seq. ID No. 164 |
| 87c | Seq. ID No. 165 | Seq. ID No. 166 |
| 89 | Seq. ID No. 169 | Seq. ID No. 170 |
| 94 | Seq. ID No. 185 | Seq. ID No. 186 |
| 97b | Seq. ID No. 189 | Seq. ID No. 190 |
| 101 | Seq. ID No. 193 | Seq. ID No. 194 |
| 119a | Seq. ID No. 229 | Seq. ID No. 230 |
| 119b | Seq. ID No. 231 | Seq. ID No. 232 |
| 132a | Seq. ID No. 255 | Seq. ID No. 256 |
| 132b | Seq. ID No. 257 | Seq. ID No. 258 |
| 132c | Seq. ID No. 259 | Seq. ID No. 260 |
| 142 | Seq. ID No. 275 | Seq. ID No. 276 |
| 163 | Seq. ID No. 337 | Seq. ID No. 338 |
| 164 | Seq. ID No. 339 | Seq. ID No. 340 |
| 169 | Seq. ID No. 347 | Seq. ID No. 348 |
| 170 | Seq. ID No. 349 | Seq. ID No. 350 |
| 173 | Seq. ID No. 357 | Seq. ID No. 358 |
| 174 | Seq. ID No. 359 | Seq. ID No. 360 |
| 179a | Seq. ID No. 369 | Seq. ID No. 370 |
| 179b | Seq. ID No. 371 | Seq. ID No. 372 |
| 183a | Seq. ID No. 377 | Seq. ID No. 378 |
| 187a | Seq. ID No. 385 | Seq. ID No. 386 |
| 187b | Seq. ID No. 387 | Seq. ID No. 388 |
| 191b | Seq. ID No. 393 | Seq. ID No. 394 |
| 193 | Seq. ID No. 399 | Seq. ID No. 400 |
| 194a | Seq. ID No. 401 | Seq. ID No. 402 |
| 194b | Seq. ID No. 403 | Seq. ID No. 404 |
| 196 | Seq. ID No. 407 | Seq. ID No. 408 |
| 201a | Seq. ID No. 419 | Seq. ID No. 420 |
| 207 | Seq. ID No. 431 | Seq. ID No. 432 |
| 235a | Seq. ID No. 477 | Seq. ID No. 478 |
| 241 | Seq. ID No. 495 | Seq. ID No. 496 |
| 247 | Seq. ID No. 507 | Seq. ID No. 508 |
| 253a | Seq. ID No. 519 | Seq. ID No. 520 |
| 260 | Seq. ID No. 541 | Seq. ID No. 542 |
| 263a | Seq. ID No. 545 | Seq. ID No. 546 |
| 263b | Seq. ID No. 547 | Seq. ID No. 548 |
| 264b | Seq. ID No. 549 | Seq. ID No. 550 |
| 268 | Seq. ID No. 557 | Seq. ID No. 558 |
| 282 | Seq. ID No. 589 | Seq. ID No. 590 |
| 297 | Seq. ID No. 617 | Seq. ID No. 618 |
| 300b | Seq. ID No. 619 | Seq. ID No. 620 |
| 309 | Seq. ID No. 633 | Seq. ID No. 634 |
| 313a | Seq. ID No. 639 | Seq. ID No. 640 |
| 313b | Seq. ID No. 641 | Seq. ID No. 642 |
| 313c | Seq. ID No. 643 | Seq. ID No. 644 |
| 313d | Seq. ID No. 645 | Seq. ID No. 646 |
| 313e | Seq. ID No. 647 | Seq. ID No. 648 |
| 316a | Seq. ID No. 651 | Seq. ID No. 652 |
| 317a | Seq. ID No. 653 | Seq. ID No. 654 |
| 317b | Seq. ID No. 655 | Seq. ID No. 656 |
| 320 | Seq. ID No. 667 | Seq. ID No. 668 |
| 331 | Seq. ID No. 691 | Seq. ID No. 692 |
| 332 | Seq. ID No. 693 | Seq. ID No. 694 |
| 333 | Seq. ID No. 695 | Seq. ID No. 696 |
| 334 | Seq. ID No. 697 | Seq. ID No. 698 |
| 342a | Seq. ID No. 715 | Seq. ID No. 716 |
| 342b | Seq. ID No. 717 | Seq. ID No. 718 |
| 342c | Seq. ID No. 719 | Seq. ID No. 720 |
| 344a | Seq. ID No. 731 | Seq. ID No. 732 |
| 344b | Seq. ID No. 733 | Seq. ID No. 734 |
| 347 | Seq. ID No. 741 | Seq. ID No. 742 |
| 349a | Seq. ID No. 743 | Seq. ID No. 744 |
| 349b | Seq. ID No. 745 | Seq. ID No. 746 |
| 349c | Seq. ID No. 747 | Seq. ID No. 748 |
| 350a | Seq. ID No. 749 | Seq. ID No. 750 |
| 350b | Seq. ID No. 751 | Seq. ID No. 752 |
| 352a | Seq. ID No. 755 | Seq. ID No. 756 |
| 352b | Seq. ID No. 757 | Seq. ID No. 758 |
| 352c | Seq. ID No. 759 | Seq. ID No. 760 |
| 354a | Seq. ID No. 761 | Seq. ID No. 762 |
| 354b | Seq. ID No. 763 | Seq. ID No. 764 |
| 354c | Seq. ID No. 765 | Seq. ID No. 766 |
| 355 | Seq. ID No. 767 | Seq. ID No. 768 |
| 372a | Seq. ID No. 801 | Seq. ID No. 802 |
| 372b | Seq. ID No. 803 | Seq. ID No. 804 |
| 372c | Seq. ID No. 805 | Seq. ID No. 806 |
| 372d | Seq. ID No. 807 | Seq. ID No. 808 |
| 374 | Seq. ID No. 809 | Seq. ID No. 810 |
| 377a | Seq. ID No. 811 | Seq. ID No. 812 |
| 377b | Seq. ID No. 813 | Seq. ID No. 814 |
| 386 | Seq. ID No. 833 | Seq. ID No. 834 |
| 393b | Seq. ID No. 841 | Seq. ID No. 842 |
| 396 | Seq. ID No. 849 | Seq. ID No. 850 |
| 398 | Seq. ID No. 851 | Seq. ID No. 852 |
| 400a | Seq. ID No. 853 | Seq. ID No. 854 |
| 400b | Seq. ID No. 855 | Seq. ID No. 856 |
| 416a | Seq. ID No. 887 | Seq. ID No. 888 |
| 416b | Seq. ID No. 889 | Seq. ID No. 890 |
| 417 | Seq. ID No. 891 | Seq. ID No. 892 |
| 420a | Seq. ID No. 903 | Seq. ID No. 904 |
| 420b | Seq. ID No. 905 | Seq. ID No. 906 |
| 422 | Seq. ID No. 909 | Seq. ID No. 910 |
| 434 | Seq. ID No. 937 | Seq. ID No. 938 |
| 437b | Seq. ID No. 941 | Seq. ID No. 942 |
| 437c | Seq. ID No. 943 | Seq. ID No. 944 |
| 440 | Seq. ID No. 951 | Seq. ID No. 952 |
| 443b | Seq. ID No. 957 | Seq. ID No. 958 |
| 451a | Seq. ID No. 973 | Seq. ID No. 974 |
| 451b | Seq. ID No. 975 | Seq. ID No. 976 |
| 454b | Seq. ID No. 987 | Seq. ID No. 988 |
| 462 | Seq. ID No. 1005 | Seq. ID No. 1006 |
| 463a | Seq. ID No. 1007 | Seq. ID No. 1008 |
| 463b | Seq. ID No. 1009 | Seq. ID No. 1010 |
| 464a | Seq. ID No. 1011 | Seq. ID No. 1012 |
| 465b | Seq. ID No. 1019 | Seq. ID No. 1020 |
| 471a | Seq. ID No. 1025 | Seq. ID No. 1026 |
| 471b | Seq. ID No. 1027 | Seq. ID No. 1028 |
| 476 | Seq. ID No. 1045 | Seq. ID No. 1046 |
| 477b | Seq. ID No. 1047 | Seq. ID No. 1048 |
| 491a | Seq. ID No. 1071 | Seq. ID No. 1072 |
| 491b | Seq. ID No. 1073 | Seq. ID No. 1074 |
| 494a | Seq. ID No. 1077 | Seq. ID No. 1078 |
| 507a | Seq. ID No. 1105 | Seq. ID No. 1106 |
| 507b | Seq. ID No. 1107 | Seq. ID No. 1108 |
| 510 | Seq. ID No. 1111 | Seq. ID No. 1112 |
| 521b | Seq. ID No. 1131 | Seq. ID No. 1132 |
| 524a | Seq. ID No. 1135 | Seq. ID No. 1136 |
| 525 | Seq. ID No. 1139 | Seq. ID No. 1140 |
| 530a | Seq. ID No. 1151 | Seq. ID No. 1152 |
| 530b | Seq. ID No. 1153 | Seq. ID No. 1154 |
| 541a | Seq. ID No. 1165 | Seq. ID No. 1166 |
| 541b | Seq. ID No. 1167 | Seq. ID No. 1168 |
| 541c | Seq. ID No. 1169 | Seq. ID No. 1170 |
| 541d | Seq. ID No. 1171 | Seq. ID No. 1172 |
| 542 | Seq. ID No. 1173 | Seq. ID No. 1174 |
| 544 | Seq. ID No. 1181 | Seq. ID No. 1182 |
| 545a | Seq. ID No. 1183 | Seq. ID No. 1184 |
| 545b | Seq. ID No. 1185 | Seq. ID No. 1186 |
| 545c | Seq. ID No. 1187 | Seq. ID No. 1188 |
| 546a | Seq. ID No. 1189 | Seq. ID No. 1190 |
| 546b | Seq. ID No. 1191 | Seq. ID No. 1192 |
| 546c | Seq. ID No. 1193 | Seq. ID No. 1194 |
| 547a | Seq. ID No. 1195 | Seq. ID No. 1196 |
| 547b | Seq. ID No. 1197 | Seq. ID No. 1198 |
| 548 | Seq. ID No. 1199 | Seq. ID No. 1200 |
| 549b | Seq. ID No. 1201 | Seq. ID No. 1202 |
| 550a | Seq. ID No. 1203 | Seq. ID No. 1204 |
| 553c | Seq. ID No. 1207 | Seq. ID No. 1208 |
| 556 | Seq. ID No. 1213 | Seq. ID No. 1214 |
| 564a | Seq. ID No. 1227 | Seq. ID No. 1228 |
| 564b | Seq. ID No. 1229 | Seq. ID No. 1230 |
| 571 | Seq. ID No. 1239 | Seq. ID No. 1240 |
| 576a | Seq. ID No. 1243 | Seq. ID No. 1244 |
| 576b | Seq. ID No. 1245 | Seq. ID No. 1246 |
| 578a | Seq. ID No. 1247 | Seq. ID No. 1248 |
| 578b | Seq. ID No. 1249 | Seq. ID No. 1250 |
| 585b | Seq. ID No. 1259 | Seq. ID No. 1260 |
| 587a | Seq. ID No. 1265 | Seq. ID No. 1266 |
| 587b | Seq. ID No. 1267 | Seq. ID No. 1268 |
| 587c | Seq. ID No. 1269 | Seq. ID No. 1270 |
| 587d | Seq. ID No. 1271 | Seq. ID No. 1272 |
| 588a | Seq. ID No. 1273 | Seq. ID No. 1274 |
| 588b | Seq. ID No. 1275 | Seq. ID No. 1276 |
| 589a | Seq. ID No. 1277 | Seq. ID No. 1278 |
| 589b | Seq. ID No. 1279 | Seq. ID No. 1280 |
| 590 | Seq. ID No. 1281 | Seq. ID No. 1282 |
| 595a | Seq. ID No. 1283 | Seq. ID No. 1284 |
| 595b | Seq. ID No. 1285 | Seq. ID No. 1286 |
| 596a | Seq. ID No. 1287 | Seq. ID No. 1288 |
| 596b | Seq. ID No. 1289 | Seq. ID No. 1290 |
| 596c | Seq. ID No. 1291 | Seq. ID No. 1292 |
| 597 | Seq. ID No. 1293 | Seq. ID No. 1294 |
| 598a | Seq. ID No. 1295 | Seq. ID No. 1296 |
| 598b | Seq. ID No. 1297 | Seq. ID No. 1298 |
| 599a | Seq. ID No. 1299 | Seq. ID No. 1300 |
| 599b | Seq. ID No. 1301 | Seq. ID No. 1302 |
| 600a | Seq. ID No. 1303 | Seq. ID No. 1304 |
| 600b | Seq. ID No. 1305 | Seq. ID No. 1306 |
| 603a | Seq. ID No. 1307 | Seq. ID No. 1308 |
| 606 | Seq. ID No. 1309 | Seq. ID No. 1310 |
| 607a | Seq. ID No. 1311 | Seq. ID No. 1312 |
| 607b | Seq. ID No. 1313 | Seq. ID No. 1314 |
| 610a | Seq. ID No. 1319 | Seq. ID No. 1320 |
| 610b | Seq. ID No. 1321 | Seq. ID No. 1322 |
| 611 | Seq. ID No. 1323 | Seq. ID No. 1324 |
| 613a | Seq. ID No. 1325 | Seq. ID No. 1326 |
| 613b | Seq. ID No. 1327 | Seq. ID No. 1328 |
| 615b | Seq. ID No. 1337 | Seq. ID No. 1338 |
| 619a | Seq. ID No. 1341 | Seq. ID No. 1342 |
| 619b | Seq. ID No. 1343 | Seq. ID No. 1344 |
| 623 | Seq. ID No. 1347 | Seq. ID No. 1348 |
| 624a | Seq. ID No. 1349 | Seq. ID No. 1350 |
| 624b | Seq. ID No. 1351 | Seq. ID No. 1352 |
| 624c | Seq. ID No. 1353 | Seq. ID No. 1354 |
| 625 | Seq. ID No. 1355 | Seq. ID No. 1356 |
| 632b | Seq. ID No. 1371 | Seq. ID No. 1372 |
| 634b | Seq. ID No. 1379 | Seq. ID No. 1380 |
| 636 | Seq. ID No. 1381 | Seq. ID No. 1382 |
| 640 | Seq. ID No. 1389 | Seq. ID No. 1390 |
| 644a | Seq. ID No. 1397 | Seq. ID No. 1398 |
| 644b | Seq. ID No. 1399 | Seq. ID No. 1400 |
| 645 | Seq. ID No. 1401 | Seq. ID No. 1402 |
| 658a | Seq. ID No. 1417 | Seq. ID No. 1418 |
| 658b | Seq. ID No. 1419 | Seq. ID No. 1420 |
| 659a | Seq. ID No. 1421 | Seq. ID No. 1422 |
| 659b | Seq. ID No. 1423 | Seq. ID No. 1424 |
| 676 | Seq. ID No. 1439 | Seq. ID No. 1440 |
| 679a | Seq. ID No. 1445 | Seq. ID No. 1446 |
| 679b | Seq. ID No. 1447 | Seq. ID No. 1448 |
| 684 | Seq. ID No. 1457 | Seq. ID No. 1458 |
| 690 | Seq. ID No. 1465 | Seq. ID No. 1466 |
| 699b | Seq. ID No. 1479 | Seq. ID No. 1480 |
| 721 | Seq. ID No. 1511 | Seq. ID No. 1512 |
| 739 | Seq. ID No. 1551 | Seq. ID No. 1552 |
| 740 | Seq. ID No. 1553 | Seq. ID No. 1554 |
| 741a | Seq. ID No. 1555 | Seq. ID No. 1556 |
| 741b | Seq. ID No. 1557 | Seq. ID No. 1558 |
| 749 | Seq. ID No. 1579 | Seq. ID No. 1580 |
| 764 | Seq. ID No. 1609 | Seq. ID No. 1610 |
| 772a | Seq. ID No. 1621 | Seq. ID No. 1622 |
| 772b | Seq. ID No. 1623 | Seq. ID No. 1624 |
| 774 | Seq. ID No. 1625 | Seq. ID No. 1626 |
| 775 | Seq. ID No. 1627 | Seq. ID No. 1628 |
| 777a | Seq. ID No. 1629 | Seq. ID No. 1630 |
| 777c | Seq. ID No. 1631 | Seq. ID No. 1632 |
| 779 | Seq. ID No. 1633 | Seq. ID No. 1634 |
| 782b | Seq. ID No. 1639 | Seq. ID No. 1640 |
| 786b | Seq. ID No. 1643 | Seq. ID No. 1644 |
| 791 | Seq. ID No. 1655 | Seq. ID No. 1656 |
| 795 | Seq. ID No. 1667 | Seq. ID No. 1668 |
| 799a | Seq. ID No. 1671 | Seq. ID No. 1672 |
| 808 | Seq. ID No. 1693 | Seq. ID No. 1694 |
| 809b | Seq. ID No. 1695 | Seq. ID No. 1696 |
| 816 | Seq. ID No. 1701 | Seq. ID No. 1702 |
| 820a | Seq. ID No. 1703 | Seq. ID No. 1704 |
| 820b | Seq. ID No. 1705 | Seq. ID No. 1706 |
| 835a | Seq. ID No. 1721 | Seq. ID No. 1722 |
| 835b | Seq. ID No. 1723 | Seq. ID No. 1724 |
| 837a | Seq. ID No. 1731 | Seq. ID No. 1732 |
| 843 | Seq. ID No. 1737 | Seq. ID No. 1738 |
| 848 | Seq. ID No. 1743 | Seq. ID No. 1744 |
| 854 | Seq. ID No. 1759 | Seq. ID No. 1760 |
| 857 | Seq. ID No. 1763 | Seq. ID No. 1764 |
| 860 | Seq. ID No. 1773 | Seq. ID No. 1774 |
| 862 | Seq. ID No. 1775 | Seq. ID No. 1776 |
| 863 | Seq. ID No. 1777 | Seq. ID No. 1778 |
| 864a | Seq. ID No. 1779 | Seq. ID No. 1780 |
| 864b | Seq. ID No. 1781 | Seq. ID No. 1782 |
| 874a | Seq. ID No. 1799 | Seq. ID No. 1800 |
| 874b | Seq. ID No. 1801 | Seq. ID No. 1802 |
| 875 | Seq. ID No. 1803 | Seq. ID No. 1804 |
| 878a | Seq. ID No. 1807 | Seq. ID No. 1808 |
| 887 | Seq. ID No. 1823 | Seq. ID No. 1824 |
| 894 | Seq. ID No. 1831 | Seq. ID No. 1832 |
| 917 | Seq. ID No. 1851 | Seq. ID No. 1852 |
| 919 | Seq. ID No. 1857 | Seq. ID No. 1858 |
| 931 | Seq. ID No. 1875 | Seq. ID No. 1876 |
| 943 | Seq. ID No. 1899 | Seq. ID No. 1900 |
| 950 | Seq. ID No. 1915 | Seq. ID No. 1916 |
| 955a | Seq. ID No. 1927 | Seq. ID No. 1928 |
| 955b | Seq. ID No. 1929 | Seq. ID No. 1930 |
| 978a | Seq. ID No. 1973 | Seq. ID No. 1974 |
| 979 | Seq. ID No. 1977 | Seq. ID No. 1978 |
| 981a | Seq. ID No. 1981 | Seq. ID No. 1982 |
| 993 | Seq. ID No. 2005 | Seq. ID No. 2006 |
| 994 | Seq. ID No. 2007 | Seq. ID No. 2008 |
| 995 | Seq. ID No. 2009 | Seq. ID No. 2010 |
| 997 | Seq. ID No. 2011 | Seq. ID No. 2012 |
| 999a | Seq. ID No. 2017 | Seq. ID No. 2018 |
| 999b | Seq. ID No. 2019 | Seq. ID No. 2020 |
| 1001 | Seq. ID No. 2023 | Seq. ID No. 2024 |
| 1003a | Seq. ID No. 2025 | Seq. ID No. 2026 |
| 1003b | Seq. ID No. 2027 | Seq. ID No. 2028 |
| 1012 | Seq. ID No. 2039 | Seq. ID No. 2040 |
| 1013a | Seq. ID No. 2041 | Seq. ID No. 2042 |
| 1013b | Seq. ID No. 2043 | Seq. ID No. 2044 |
| 1013c | Seq. ID No. 2045 | Seq. ID No. 2046 |
| 1017a | Seq. ID No. 2049 | Seq. ID No. 2050 |
| 1035a | Seq. ID No. 2081 | Seq. ID No. 2082 |
| 1035b | Seq. ID No. 2083 | Seq. ID No. 2084 |
| 1035c | Seq. ID No. 2085 | Seq. ID No. 2086 |
| 1044a | Seq. ID No. 2105 | Seq. ID No. 2106 |
| 1044b | Seq. ID No. 2107 | Seq. ID No. 2108 |
| 1044c | Seq. ID No. 2109 | Seq. ID No. 2110 |
| 1044d | Seq. ID No. 2111 | Seq. ID No. 2112 |
| 1055 | Seq. ID No. 2129 | Seq. ID No. 2130 |
| 1056a | Seq. ID No. 2131 | Seq. ID No. 2132 |
| 1056b | Seq. ID No. 2133 | Seq. ID No. 2134 |
| 1063 | Seq. ID No. 2145 | Seq. ID No. 2146 |
| 1066a | Seq. ID No. 2155 | Seq. ID No. 2156 |
| 1066b | Seq. ID No. 2157 | Seq. ID No. 2158 |
| 1067 | Seq. ID No. 2159 | Seq. ID No. 2160 |
| 1069 | Seq. ID No. 2161 | Seq. ID No. 2162 |
| 1077a | Seq. ID No. 2171 | Seq. ID No. 2172 |
| 1081a | Seq. ID No. 2187 | Seq. ID No. 2188 |
| 1081b | Seq. ID No. 2189 | Seq. ID No. 2190 |
| 1091a | Seq. ID No. 2201 | Seq. ID No. 2202 |
| 1091b | Seq. ID No. 2203 | Seq. ID No. 2204 |
| 1095a | Seq. ID No. 2209 | Seq. ID No. 2210 |
| 1095b | Seq. ID No. 2211 | Seq. ID No. 2212 |
| 1097 | Seq. ID No. 2219 | Seq. ID No. 2220 |
| 1101a | Seq. ID No. 2225 | Seq. ID No. 2226 |
| 1112a | Seq. ID No. 2243 | Seq. ID No. 2244 |
| 1125 | Seq. ID No. 2245 | Seq. ID No. 2246 |
| 1126a | Seq. ID No. 2247 | Seq. ID No. 2248 |
| 1126b | Seq. ID No. 2249 | Seq. ID No. 2250 |
| 1126c | Seq. ID No. 2251 | Seq. ID No. 2252 |
| 1128 | Seq. ID No. 2255 | Seq. ID No. 2256 |
| 1141a | Seq. ID No. 2277 | Seq. ID No. 2278 |
| 1141b | Seq. ID No. 2279 | Seq. ID No. 2280 |
| 1162 | Seq. ID No. 2341 | Seq. ID No. 2342 |
| 1168 | Seq. ID No. 2351 | Seq. ID No. 2352 |
| 1169a | Seq. ID No. 2353 | Seq. ID No. 2354 |
| 1169b | Seq. ID No. 2355 | Seq. ID No. 2356 |
| 1169c | Seq. ID No. 2357 | Seq. ID No. 2358 |
| 1170 | Seq. ID No. 2359 | Seq. ID No. 2360 |
| 1172 | Seq. ID No. 2365 | Seq. ID No. 2366 |
| 1173 | Seq. ID No. 2367 | Seq. ID No. 2368 |
| 1175 | Seq. ID No. 2371 | Seq. ID No. 2372 |
| 1178 | Seq. ID No. 2375 | Seq. ID No. 2376 |
| 1182a | Seq. ID No. 2393 | Seq. ID No. 2394 |
| 1182b | Seq. ID No. 2395 | Seq. ID No. 2396 |
| 1196a | Seq. ID No. 2425 | Seq. ID No. 2426 |
| 1196b | Seq. ID No. 2427 | Seq. ID No. 2428 |
| 1198 | Seq. ID No. 2431 | Seq. ID No. 2432 |
| 1201 | Seq. ID No. 2439 | Seq. ID No. 2440 |
| 1202a | Seq. ID No. 2441 | Seq. ID No. 2442 |
| 1202b | Seq. ID No. 2443 | Seq. ID No. 2444 |
| 1206 | Seq. ID No. 2447 | Seq. ID No. 2448 |
| 1212 | Seq. ID No. 2459 | Seq. ID No. 2460 |
| 1220 | Seq. ID No. 2479 | Seq. ID No. 2480 |
| 1225 | Seq. ID No. 2493 | Seq. ID No. 2494 |
| 1229b | Seq. ID No. 2501 | Seq. ID No. 2502 |
| 1229c | Seq. ID No. 2503 | Seq. ID No. 2504 |
| 1235 | Seq. ID No. 2521 | Seq. ID No. 2522 |
| 1236a | Seq. ID No. 2523 | Seq. ID No. 2524 |
| 1239 | Seq. ID No. 2527 | Seq. ID No. 2528 |
| 1260 | Seq. ID No. 2591 | Seq. ID No. 2592 |
| 1267a | Seq. ID No. 2601 | Seq. ID No. 2602 |
| 1267b | Seq. ID No. 2603 | Seq. ID No. 2604 |
| 1270 | Seq. ID No. 2611 | Seq. ID No. 2612 |
| 1278a | Seq. ID No. 2627 | Seq. ID No. 2628 |
| 1278b | Seq. ID No. 2629 | Seq. ID No. 2630 |
| 1289 | Seq. ID No. 2653 | Seq. ID No. 2654 |
| 1294a | Seq. ID No. 2661 | Seq. ID No. 2662 |
| 1314b | Seq. ID No. 2691 | Seq. ID No. 2692 |
| 1316 | Seq. ID No. 2695 | Seq. ID No. 2696 |
| 1317 | Seq. ID No. 2697 | Seq. ID No. 2698 |
| 1323b | Seq. ID No. 2717 | Seq. ID No. 2718 |
| 1334a | Seq. ID No. 2729 | Seq. ID No. 2730 |
| 1334b | Seq. ID No. 2731 | Seq. ID No. 2732 |
| 1335a | Seq. ID No. 2733 | Seq. ID No. 2734 |
| 1335b | Seq. ID No. 2735 | Seq. ID No. 2736 |
| 1338 | Seq. ID No. 2739 | Seq. ID No. 2740 |
| 1340a | Seq. ID No. 2741 | Seq. ID No. 2742 |
| 1340b | Seq. ID No. 2743 | Seq. ID No. 2744 |
| 1364a | Seq. ID No. 2777 | Seq. ID No. 2778 |
| 1364b | Seq. ID No. 2779 | Seq. ID No. 2780 |
| 1364c | Seq. ID No. 2781 | Seq. ID No. 2782 |
| 1365b | Seq. ID No. 2783 | Seq. ID No. 2784 |
| 1375 | Seq. ID No. 2801 | Seq. ID No. 2802 |
| 1378 | Seq. ID No. 2805 | Seq. ID No. 2806 |
| 1379 | Seq. ID No. 2807 | Seq. ID No. 2808 |
| 1380 | Seq. ID No. 2809 | Seq. ID No. 2810 |
| 1381a | Seq. ID No. 2811 | Seq. ID No. 2812 |
| 1381b | Seq. ID No. 2813 | Seq. ID No. 2814 |
| 1382 | Seq. ID No. 2815 | Seq. ID No. 2816 |
| 1383 | Seq. ID No. 2817 | Seq. ID No. 2818 |
| 1384a | Seq. ID No. 2819 | Seq. ID No. 2820 |
| 1387a | Seq. ID No. 2829 | Seq. ID No. 2830 |
| 1388b | Seq. ID No. 2831 | Seq. ID No. 2832 |
| 1389a | Seq. ID No. 2833 | Seq. ID No. 2834 |
| 1393a | Seq. ID No. 2835 | Seq. ID No. 2836 |
| 1393b | Seq. ID No. 2837 | Seq. ID No. 2838 |
| 1393c | Seq. ID No. 2839 | Seq. ID No. 2840 |
| 1393d | Seq. ID No. 2841 | Seq. ID No. 2842 |
| 1395a | Seq. ID No. 2845 | Seq. ID No. 2846 |
| 1397 | Seq. ID No. 2849 | Seq. ID No. 2850 |
| 1398 | Seq. ID No. 2851 | Seq. ID No. 2852 |
| 1399a | Seq. ID No. 2853 | Seq. ID No. 2854 |
| 1399b | Seq. ID No. 2855 | Seq. ID No. 2856 |
| 1400 | Seq. ID No. 2857 | Seq. ID No. 2858 |
| 1401 | Seq. ID No. 2859 | Seq. ID No. 2860 |
| 1402c | Seq. ID No. 2861 | Seq. ID No. 2862 |
| 1407b | Seq. ID No. 2865 | Seq. ID No. 2866 |
| 1411a | Seq. ID No. 2873 | Seq. ID No. 2874 |
| 1411b | Seq. ID No. 2875 | Seq. ID No. 2876 |
| 1411c | Seq. ID No. 2877 | Seq. ID No. 2878 |
| 1414a | Seq. ID No. 2883 | Seq. ID No. 2884 |
| 1414b | Seq. ID No. 2885 | Seq. ID No. 2886 |
| 1414c | Seq. ID No. 2887 | Seq. ID No. 2888 |
| 1415 | Seq. ID No. 2889 | Seq. ID No. 2890 |
| 1418 | Seq. ID No. 2893 | Seq. ID No. 2894 |
| 1419 | Seq. ID No. 2895 | Seq. ID No. 2896 |
| 1423 | Seq. ID No. 2903 | Seq. ID No. 2904 |
| 1427a | Seq. ID No. 2909 | Seq. ID No. 2910 |
| 1438 | Seq. ID No. 2935 | Seq. ID No. 2936 |
| 1439a | Seq. ID No. 2937 | Seq. ID No. 2938 |
| 1439b | Seq. ID No. 2939 | Seq. ID No. 2940 |
| 1439c | Seq. ID No. 2941 | Seq. ID No. 2942 |
| 1439d | Seq. ID No. 2943 | Seq. ID No. 2944 |
| 1439e | Seq. ID No. 2945 | Seq. ID No. 2946 |
| 1440a | Seq. ID No. 2947 | Seq. ID No. 2948 |
| 1440b | Seq. ID No. 2949 | Seq. ID No. 2950 |
| 1440c | Seq. ID No. 2951 | Seq. ID No. 2952 |
| 1440d | Seq. ID No. 2953 | Seq. ID No. 2954 |
| 1446a | Seq. ID No. 2965 | Seq. ID No. 2966 |
| 1446b | Seq. ID No. 2967 | Seq. ID No. 2968 |
| 1448 | Seq. ID No. 2973 | Seq. ID No. 2974 |
| 1449a | Seq. ID No. 2975 | Seq. ID No. 2976 |
| 1449b | Seq. ID No. 2977 | Seq. ID No. 2978 |
| 1452a | Seq. ID No. 2981 | Seq. ID No. 2982 |
| 1452b | Seq. ID No. 2983 | Seq. ID No. 2984 |
| 1453 | Seq. ID No. 2985 | Seq. ID No. 2986 |
| 1454 | Seq. ID No. 2987 | Seq. ID No. 2988 |
| 1455a | Seq. ID No. 2989 | Seq. ID No. 2990 |
| 1455b | Seq. ID No. 2991 | Seq. ID No. 2992 |
| 1459 | Seq. ID No. 3001 | Seq. ID No. 3002 |
| 1463a | Seq. ID No. 3007 | Seq. ID No. 3008 |
| 1463b | Seq. ID No. 3009 | Seq. ID No. 3010 |
| 1463c | Seq. ID No. 3011 | Seq. ID No. 3012 |
| 1467 | Seq. ID No. 3021 | Seq. ID No. 3022 |
| 1469a | Seq. ID No. 3023 | Seq. ID No. 3024 |
| 1474 | Seq. ID No. 3041 | Seq. ID No. 3042 |
| 1476 | Seq. ID No. 3045 | Seq. ID No. 3046 |
| 1487 | Seq. ID No. 3071 | Seq. ID No. 3072 |
| 1489 | Seq. ID No. 3087 | Seq. ID No. 3088 |
| 1502a | Seq. ID No. 3105 | Seq. ID No. 3106 |
| 1502b | Seq. ID No. 3107 | Seq. ID No. 3108 |
| 1507a | Seq. ID No. 3119 | Seq. ID No. 3120 |
| 1507b | Seq. ID No. 3121 | Seq. ID No. 3122 |
| 1507c | Seq. ID No. 3123 | Seq. ID No. 3124 |
| 1518a | Seq. ID No. 3151 | Seq. ID No. 3152 |
| 1518b | Seq. ID No. 3153 | Seq. ID No. 3154 |
| 1533a | Seq. ID No. 3167 | Seq. ID No. 3168 |
| 1535a | Seq. ID No. 3173 | Seq. ID No. 3174 |
| 1536a | Seq. ID No. 3175 | Seq. ID No. 3176 |
| 1536b | Seq. ID No. 3177 | Seq. ID No. 3178 |
| 1542 | Seq. ID No. 3181 | Seq. ID No. 3182 |
| 1552 | Seq. ID No. 3193 | Seq. ID No. 3194 |
| 1557a | Seq. ID No. 3213 | Seq. ID No. 3214 |
| 1557b | Seq. ID No. 3215 | Seq. ID No. 3216 |
| 1557c | Seq. ID No. 3217 | Seq. ID No. 3218 |
| 1559a | Seq. ID No. 3219 | Seq. ID No. 3220 |
| 1559b | Seq. ID No. 3221 | Seq. ID No. 3222 |
| 1563 | Seq. ID No. 3223 | Seq. ID No. 3224 |
| 1567 | Seq. ID No. 3231 | Seq. ID No. 3232 |
| 1568b | Seq. ID No. 3233 | Seq. ID No. 3234 |
| 1569 | Seq. ID No. 3235 | Seq. ID No. 3236 |
| 1570 | Seq. ID No. 3237 | Seq. ID No. 3238 |
| 1571 | Seq. ID No. 3239 | Seq. ID No. 3240 |
| 1572a | Seq. ID No. 3241 | Seq. ID No. 3242 |
| 1572b | Seq. ID No. 3243 | Seq. ID No. 3244 |
| 1572c | Seq. ID No. 3245 | Seq. ID No. 3246 |
| 1572d | Seq. ID No. 3247 | Seq. ID No. 3248 |
| 1572e | Seq. ID No. 3249 | Seq. ID No. 3250 |
| 1580a | Seq. ID No. 3261 | Seq. ID No. 3262 |
| 1580b | Seq. ID No. 3263 | Seq. ID No. 3264 |
| 1581 | Seq. ID No. 3265 | Seq. ID No. 3266 |
| 1582a | Seq. ID No. 3267 | Seq. ID No. 3268 |
| 1582b | Seq. ID No. 3269 | Seq. ID No. 3270 |
| 1582c | Seq. ID No. 3271 | Seq. ID No. 3272 |
| 1584 | Seq. ID No. 3273 | Seq. ID No. 3274 |
| 1585a | Seq. ID No. 3275 | Seq. ID No. 3276 |
| 1585b | Seq. ID No. 3277 | Seq. ID No. 3278 |
| 1608 | Seq. ID No. 3325 | Seq. ID No. 3326 |
| 1612 | Seq. ID No. 3331 | Seq. ID No. 3332 |
| 1621 | Seq. ID No. 3339 | Seq. ID No. 3340 |
| 1622a | Seq. ID No. 3341 | Seq. ID No. 3342 |
| 1624 | Seq. ID No. 3349 | Seq. ID No. 3350 |
| 1628a | Seq. ID No. 3361 | Seq. ID No. 3362 |
| 1628b | Seq. ID No. 3363 | Seq. ID No. 3364 |
| 1633a | Seq. ID No. 3369 | Seq. ID No. 3370 |
| 1633b | Seq. ID No. 3371 | Seq. ID No. 3372 |
| 1646a | Seq. ID No. 3389 | Seq. ID No. 3390 |
| 1648b | Seq. ID No. 3403 | Seq. ID No. 3404 |
| 1656b | Seq. ID No. 3417 | Seq. ID No. 3418 |
| 1657 | Seq. ID No. 3419 | Seq. ID No. 3420 |
| 1659a | Seq. ID No. 3421 | Seq. ID No. 3422 |
| 1663 | Seq. ID No. 3433 | Seq. ID No. 3434 |
| 1665a | Seq. ID No. 3437 | Seq. ID No. 3438 |
| 1669 | Seq. ID No. 3441 | Seq. ID No. 3442 |
| 1670a | Seq. ID No. 3443 | Seq. ID No. 3444 |
| 1670b | Seq. ID No. 3445 | Seq. ID No. 3446 |
| 1670c | Seq. ID No. 3447 | Seq. ID No. 3448 |
| 1679 | Seq. ID No. 3473 | Seq. ID No. 3474 |
| 1683a | Seq. ID No. 3481 | Seq. ID No. 3482 |
| 1683b | Seq. ID No. 3483 | Seq. ID No. 3484 |
| 1687 | Seq. ID No. 3489 | Seq. ID No. 3490 |
| 1690a | Seq. ID No. 3503 | Seq. ID No. 3504 |
| 1690b | Seq. ID No. 3505 | Seq. ID No. 3506 |
| 1692 | Seq. ID No. 3509 | Seq. ID No. 3510 |
| 1693 | Seq. ID No. 3511 | Seq. ID No. 3512 |
| 1697a | Seq. ID No. 3523 | Seq. ID No. 3524 |
| 1697b | Seq. ID No. 3525 | Seq. ID No. 3526 |
| 1700a | Seq. ID No. 3531 | Seq. ID No. 3532 |
| 1700b | Seq. ID No. 3533 | Seq. ID No. 3534 |
| 1703b | Seq. ID No. 3539 | Seq. ID No. 3540 |
| 1704a | Seq. ID No. 3541 | Seq. ID No. 3542 |
| 1704b | Seq. ID No. 3543 | Seq. ID No. 3544 |
| 1705a | Seq. ID No. 3545 | Seq. ID No. 3546 |
| 1705b | Seq. ID No. 3547 | Seq. ID No. 3548 |
| 1708 | Seq. ID No. 3555 | Seq. ID No. 3556 |
| 1709 | Seq. ID No. 3557 | Seq. ID No. 3558 |
| 1712a | Seq. ID No. 3563 | Seq. ID No. 3564 |
| 1712b | Seq. ID No. 3565 | Seq. ID No. 3566 |
| 1718a | Seq. ID No. 3571 | Seq. ID No. 3572 |
| 1718b | Seq. ID No. 3573 | Seq. ID No. 3574 |
| 1724 | Seq. ID No. 3581 | Seq. ID No. 3582 |
| 1731 | Seq. ID No. 3587 | Seq. ID No. 3588 |
| 1734 | Seq. ID No. 3591 | Seq. ID No. 3592 |
| 1735a | Seq. ID No. 3593 | Seq. ID No. 3594 |
| 1735b | Seq. ID No. 3595 | Seq. ID No. 3596 |
| 1743 | Seq. ID No. 3611 | Seq. ID No. 3612 |
| 1744a | Seq. ID No. 3613 | Seq. ID No. 3614 |
| 1745 | Seq. ID No. 3617 | Seq. ID No. 3618 |
| 1747 | Seq. ID No. 3619 | Seq. ID No. 3620 |
| 1749a | Seq. ID No. 3621 | Seq. ID No. 3622 |
| 1749b | Seq. ID No. 3623 | Seq. ID No. 3624 |
| 1750a | Seq. ID No. 3625 | Seq. ID No. 3626 |
| 1753 | Seq. ID No. 3631 | Seq. ID No. 3632 |
| 1757a | Seq. ID No. 3637 | Seq. ID No. 3638 |
| 1757b | Seq. ID No. 3639 | Seq. ID No. 3640 |
| 1766 | Seq. ID No. 3655 | Seq. ID No. 3656 |
| 1767a | Seq. ID No. 3657 | Seq. ID No. 3658 |
| 1767b | Seq. ID No. 3659 | Seq. ID No. 3660 |
| 1769a | Seq. ID No. 3661 | Seq. ID No. 3662 |
| 1778a | Seq. ID No. 3677 | Seq. ID No. 3678 |
| 1778b | Seq. ID No. 3679 | Seq. ID No. 3680 |
| 1783a | Seq. ID No. 3683 | Seq. ID No. 3684 |
| 1783b | Seq. ID No. 3685 | Seq. ID No. 3686 |
| 1790a | Seq. ID No. 3701 | Seq. ID No. 3702 |
| 1790b | Seq. ID No. 3703 | Seq. ID No. 3704 |
| 1794a | Seq. ID No. 3717 | Seq. ID No. 3718 |
| 1797a | Seq. ID No. 3719 | Seq. ID No. 3720 |
| 1797b | Seq. ID No. 3721 | Seq. ID No. 3722 |
| 1798a | Seq. ID No. 3723 | Seq. ID No. 3724 |
| 1800 | Seq. ID No. 3729 | Seq. ID No. 3730 |
| 1802a | Seq. ID No. 3731 | Seq. ID No. 3732 |
| 1802b | Seq. ID No. 3733 | Seq. ID No. 3734 |
| 1803 | Seq. ID No. 3735 | Seq. ID No. 3736 |
| 1804a | Seq. ID No. 3737 | Seq. ID No. 3738 |
| 1804b | Seq. ID No. 3739 | Seq. ID No. 3740 |
| 1805 | Seq. ID No. 3741 | Seq. ID No. 3742 |
| 1807a | Seq. ID No. 3749 | Seq. ID No. 3750 |
| 1807b | Seq. ID No. 3751 | Seq. ID No. 3752 |
| 1808a | Seq. ID No. 3753 | Seq. ID No. 3754 |
| 1808b | Seq. ID No. 3755 | Seq. ID No. 3756 |
| 1809a | Seq. ID No. 3761 | Seq. ID No. 3762 |
| 1812a | Seq. ID No. 3781 | Seq. ID No. 3782 |
| 1812b | Seq. ID No. 3783 | Seq. ID No. 3784 |
| 1813 | Seq. ID No. 3785 | Seq. ID No. 3786 |
| 1815a | Seq. ID No. 3787 | Seq. ID No. 3788 |
| 1815b | Seq. ID No. 3789 | Seq. ID No. 3790 |
| 1815c | Seq. ID No. 3791 | Seq. ID No. 3792 |
| 1817a | Seq. ID No. 3799 | Seq. ID No. 3800 |
| 1822 | Seq. ID No. 3807 | Seq. ID No. 3808 |
| 1823a | Seq. ID No. 3809 | Seq. ID No. 3810 |
| 1823b | Seq. ID No. 3811 | Seq. ID No. 3812 |
| 1825 | Seq. ID No. 3813 | Seq. ID No. 3814 |
| 1826a | Seq. ID No. 3815 | Seq. ID No. 3816 |
| 1826b | Seq. ID No. 3817 | Seq. ID No. 3818 |
| 1834 | Seq. ID No. 3827 | Seq. ID No. 3828 |
| 1835 | Seq. ID No. 3829 | Seq. ID No. 3830 |
| 1836a | Seq. ID No. 3831 | Seq. ID No. 3832 |
| 1836b | Seq. ID No. 3833 | Seq. ID No. 3834 |
| 1837 | Seq. ID No. 3835 | Seq. ID No. 3836 |
| 1842a | Seq. ID No. 3849 | Seq. ID No. 3850 |
| 1854a | Seq. ID No. 3879 | Seq. ID No. 3880 |
| 1854b | Seq. ID No. 3881 | Seq. ID No. 3882 |
| 1858 | Seq. ID No. 3895 | Seq. ID No. 3896 |
| 1862a | Seq. ID No. 3903 | Seq. ID No. 3904 |
| 1864a | Seq. ID No. 3909 | Seq. ID No. 3910 |
| 1868 | Seq. ID No. 3915 | Seq. ID No. 3916 |
| 1882 | Seq. ID No. 3939 | Seq. ID No. 3940 |
| 1894c | Seq. ID No. 3959 | Seq. ID No. 3960 |
| 1895 | Seq. ID No. 3961 | Seq. ID No. 3962 |
| 1896a | Seq. ID No. 3963 | Seq. ID No. 3964 |
| 1896b | Seq. ID No. 3965 | Seq. ID No. 3966 |
| 1898a | Seq. ID No. 3973 | Seq. ID No. 3974 |
| 1899 | Seq. ID No. 3977 | Seq. ID No. 3978 |
| 1900 | Seq. ID No. 3979 | Seq. ID No. 3980 |
| 1916 | Seq. ID No. 3993 | Seq. ID No. 3994 |
| 1917a | Seq. ID No. 3995 | Seq. ID No. 3996 |
| 1917b | Seq. ID No. 3997 | Seq. ID No. 3998 |
| 1919a | Seq. ID No. 4005 | Seq. ID No. 4006 |
| 1919b | Seq. ID No. 4007 | Seq. ID No. 4008 |
| 1919c | Seq. ID No. 4009 | Seq. ID No. 4010 |
| 1937a | Seq. ID No. 4041 | Seq. ID No. 4042 |
| 1937b | Seq. ID No. 4043 | Seq. ID No. 4044 |
| 1937c | Seq. ID No. 4045 | Seq. ID No. 4046 |
| 1944 | Seq. ID No. 4069 | Seq. ID No. 4070 |
| 1949 | Seq. ID No. 4077 | Seq. ID No. 4078 |
| 1956 | Seq. ID No. 4097 | Seq. ID No. 4098 |
| 1961 | Seq. ID No. 4113 | Seq. ID No. 4114 |
| 1966 | Seq. ID No. 4123 | Seq. ID No. 4124 |
| 1970a | Seq. ID No. 4131 | Seq. ID No. 4132 |
| 1970b | Seq. ID No. 4133 | Seq. ID No. 4134 |
| 1972 | Seq. ID No. 4135 | Seq. ID No. 4136 |
| 1976 | Seq. ID No. 4139 | Seq. ID No. 4140 |
| 1980a | Seq. ID No. 4151 | Seq. ID No. 4152 |
| 1980b | Seq. ID No. 4153 | Seq. ID No. 4154 |
| 1981 | Seq. ID No. 4155 | Seq. ID No. 4156 |
| 1991 | Seq. ID No. 4181 | Seq. ID No. 4182 |
| 2016a | Seq. ID No. 4221 | Seq. ID No. 4222 |
| 2023a | Seq. ID No. 4241 | Seq. ID No. 4242 |
| 2025 | Seq. ID No. 4253 | Seq. ID No. 4254 |
| 2030a | Seq. ID No. 4257 | Seq. ID No. 4258 |
| 2030b | Seq. ID No. 4259 | Seq. ID No. 4260 |
| 2030c | Seq. ID No. 4261 | Seq. ID No. 4262 |
| 2030d | Seq. ID No. 4263 | Seq. ID No. 4264 |
| 2032c | Seq. ID No. 4265 | Seq. ID No. 4266 |
| 2035b | Seq. ID No. 4269 | Seq. ID No. 4270 |
| 2038a | Seq. ID No. 4275 | Seq. ID No. 4276 |
| 2040a | Seq. ID No. 4277 | Seq. ID No. 4278 |
| 2042 | Seq. ID No. 4279 | Seq. ID No. 4280 |
| 2043b | Seq. ID No. 4281 | Seq. ID No. 4282 |
| 2045a | Seq. ID No. 4285 | Seq. ID No. 4286 |
| 2045b | Seq. ID No. 4287 | Seq. ID No. 4288 |
| 2049c | Seq. ID No. 4297 | Seq. ID No. 4298 |
| 2055 | Seq. ID No. 4305 | Seq. ID No. 4306 |
| 2056 | Seq. ID No. 4307 | Seq. ID No. 4308 |
| 2081a | Seq. ID No. 4363 | Seq. ID No. 4364 |
| 2081b | Seq. ID No. 4365 | Seq. ID No. 4366 |
| 2087a | Seq. ID No. 4371 | Seq. ID No. 4372 |
| 2087b | Seq. ID No. 4373 | Seq. ID No. 4374 |
| 2098b | Seq. ID No. 4395 | Seq. ID No. 4396 |
| 2101 | Seq. ID No. 4399 | Seq. ID No. 4400 |
| 2105 | Seq. ID No. 4403 | Seq. ID No. 4404 |
| 2114b | Seq. ID No. 4425 | Seq. ID No. 4426 |
| 2120a | Seq. ID No. 4437 | Seq. ID No. 4438 |
| 2120b | Seq. ID No. 4439 | Seq. ID No. 4440 |
| 2122b | Seq. ID No. 4441 | Seq. ID No. 4442 |
| 2127a | Seq. ID No. 4453 | Seq. ID No. 4454 |
| 2127b | Seq. ID No. 4455 | Seq. ID No. 4456 |
| 2159a | Seq. ID No. 4513 | Seq. ID No. 4514 |
| 2159b | Seq. ID No. 4515 | Seq. ID No. 4516 |
| 2175 | Seq. ID No. 4551 | Seq. ID No. 4552 |
| 2177b | Seq. ID No. 4561 | Seq. ID No. 4562 |
| 2182 | Seq. ID No. 4573 | Seq. ID No. 4574 |
| 2183 | Seq. ID No. 4575 | Seq. ID No. 4576 |
| 2190a | Seq. ID No. 4587 | Seq. ID No. 4588 |
| 2190b | Seq. ID No. 4589 | Seq. ID No. 4590 |
| 2193 | Seq. ID No. 4591 | Seq. ID No. 4592 |
| 2194a | Seq. ID No. 4593 | Seq. ID No. 4594 |
| 2194c | Seq. ID No. 4595 | Seq. ID No. 4596 |
| 2208a | Seq. ID No. 4617 | Seq. ID No. 4618 |
| 2208b | Seq. ID No. 4619 | Seq. ID No. 4620 |
| 2208c | Seq. ID No. 4621 | Seq. ID No. 4622 |
| 2212a | Seq. ID No. 4625 | Seq. ID No. 4626 |
| 2212b | Seq. ID No. 4627 | Seq. ID No. 4628 |
| 2216a | Seq. ID No. 4633 | Seq. ID No. 4634 |
| 2216b | Seq. ID No. 4635 | Seq. ID No. 4636 |
| 2218 | Seq. ID No. 4637 | Seq. ID No. 4638 |
| 2219a | Seq. ID No. 4639 | Seq. ID No. 4640 |
| 2225 | Seq. ID No. 4659 | Seq. ID No. 4660 |
| 2236a | Seq. ID No. 4681 | Seq. ID No. 4682 |
| 2239b | Seq. ID No. 4687 | Seq. ID No. 4688 |
| 2243a | Seq. ID No. 4695 | Seq. ID No. 4696 |
| 2245 | Seq. ID No. 4699 | Seq. ID No. 4700 |
| 2246 | Seq. ID No. 4701 | Seq. ID No. 4702 |
| 2257 | Seq. ID No. 4721 | Seq. ID No. 4722 |
| 2264a | Seq. ID No. 4735 | Seq. ID No. 4736 |
| 2264b | Seq. ID No. 4737 | Seq. ID No. 4738 |
| 2268 | Seq. ID No. 4741 | Seq. ID No. 4742 |
| 2285 | Seq. ID No. 4783 | Seq. ID No. 4784 |
| 2288 | Seq. ID No. 4789 | Seq. ID No. 4790 |
| 2294 | Seq. ID No. 4805 | Seq. ID No. 4806 |
| 2299 | Seq. ID No. 4811 | Seq. ID No. 4812 |
| 2302 | Seq. ID No. 4817 | Seq. ID No. 4818 |
| 2303 | Seq. ID No. 4819 | Seq. ID No. 4820 |
| 2304 | Seq. ID No. 4821 | Seq. ID No. 4822 |
| 2306c | Seq. ID No. 4823 | Seq. ID No. 4824 |
| 2310 | Seq. ID No. 4835 | Seq. ID No. 4836 |
| 2320 | Seq. ID No. 4853 | Seq. ID No. 4854 |
| 2336 | Seq. ID No. 4885 | Seq. ID No. 4886 |
| 2350a | Seq. ID No. 4913 | Seq. ID No. 4914 |
| 2360 | Seq. ID No. 4923 | Seq. ID No. 4924 |
| 2371a | Seq. ID No. 4949 | Seq. ID No. 4950 |
| 2372 | Seq. ID No. 4951 | Seq. ID No. 4952 |
| 2382c | Seq. ID No. 4975 | Seq. ID No. 4976 |
| 2386b | Seq. ID No. 4981 | Seq. ID No. 4982 |
| 2386c | Seq. ID No. 4983 | Seq. ID No. 4984 |
| 2390 | Seq. ID No. 4987 | Seq. ID No. 4988 |
| 2394 | Seq. ID No. 4993 | Seq. ID No. 4994 |
| 2397 | Seq. ID No. 4999 | Seq. ID No. 5000 |
| 2398 | Seq. ID No. 5001 | Seq. ID No. 5002 |
| 2402a | Seq. ID No. 5011 | Seq. ID No. 5012 |
| 2402b | Seq. ID No. 5013 | Seq. ID No. 5014 |
| 2407a | Seq. ID No. 5023 | Seq. ID No. 5024 |
| 2412 | Seq. ID No. 5031 | Seq. ID No. 5032 |
| 2413a | Seq. ID No. 5033 | Seq. ID No. 5034 |
| 2413b | Seq. ID No. 5035 | Seq. ID No. 5036 |
| 2415a | Seq. ID No. 5037 | Seq. ID No. 5038 |
| 2416b | Seq. ID No. 5041 | Seq. ID No. 5042 |
| 2416c | Seq. ID No. 5043 | Seq. ID No. 5044 |
| 2420 | Seq. ID No. 5053 | Seq. ID No. 5054 |
| 2423a | Seq. ID No. 5061 | Seq. ID No. 5062 |
| 2423b | Seq. ID No. 5063 | Seq. ID No. 5064 |
| 2434 | Seq. ID No. 5085 | Seq. ID No. 5086 |
| 2443 | Seq. ID No. 5093 | Seq. ID No. 5094 |
| 2463a | Seq. ID No. 5151 | Seq. ID No. 5152 |
| 2464 | Seq. ID No. 5153 | Seq. ID No. 5154 |
| 2468a | Seq. ID No. 5159 | Seq. ID No. 5160 |
| 2468b | Seq. ID No. 5161 | Seq. ID No. 5162 |
| 2470 | Seq. ID No. 5167 | Seq. ID No. 5168 |
| 2474a | Seq. ID No. 5173 | Seq. ID No. 5174 |
| 2474b | Seq. ID No. 5175 | Seq. ID No. 5176 |
| 2474c | Seq. ID No. 5177 | Seq. ID No. 5178 |
| 2481a | Seq. ID No. 5189 | Seq. ID No. 5190 |
| 2481b | Seq. ID No. 5191 | Seq. ID No. 5192 |
| 2482 | Seq. ID No. 5193 | Seq. ID No. 5194 |
| 2484a | Seq. ID No. 5197 | Seq. ID No. 5198 |
| 2484b | Seq. ID No. 5199 | Seq. ID No. 5200 |
| 2498b | Seq. ID No. 5221 | Seq. ID No. 5222 |
| 2500a | Seq. ID No. 5223 | Seq. ID No. 5224 |
| 2500b | Seq. ID No. 5225 | Seq. ID No. 5226 |
| 2500c | Seq. ID No. 5227 | Seq. ID No. 5228 |
| 2505 | Seq. ID No. 5237 | Seq. ID No. 5238 |
| 2518a | Seq. ID No. 5267 | Seq. ID No. 5268 |
| 2519a | Seq. ID No. 5269 | Seq. ID No. 5270 |
| 2519b | Seq. ID No. 5271 | Seq. ID No. 5272 |
| 2521 | Seq. ID No. 5281 | Seq. ID No. 5282 |
| 2532b | Seq. ID No. 5301 | Seq. ID No. 5302 |
| 2532c | Seq. ID No. 5303 | Seq. ID No. 5304 |
| 2542a | Seq. ID No. 5315 | Seq. ID No. 5316 |
| 2543 | Seq. ID No. 5321 | Seq. ID No. 5322 |
| 2546 | Seq. ID No. 5327 | Seq. ID No. 5328 |
| 2553 | Seq. ID No. 5347 | Seq. ID No. 5348 |
| 2567a | Seq. ID No. 5385 | Seq. ID No. 5386 |
| 2605a | Seq. ID No. 5445 | Seq. ID No. 5446 |
| 2605b | Seq. ID No. 5447 | Seq. ID No. 5448 |
| 2616a | Seq. ID No. 5459 | Seq. ID No. 5460 |
| 2616d | Seq. ID No. 5465 | Seq. ID No. 5466 |
| 2616e | Seq. ID No. 5467 | Seq. ID No. 5468 |
| 2618a | Seq. ID No. 5479 | Seq. ID No. 5480 |
| 2618b | Seq. ID No. 5481 | Seq. ID No. 5482 |
| 2620 | Seq. ID No. 5485 | Seq. ID No. 5486 |
| 2621a | Seq. ID No. 5487 | Seq. ID No. 5488 |
| 2631 | Seq. ID No. 5515 | Seq. ID No. 5516 |
| 2632 | Seq. ID No. 5517 | Seq. ID No. 5518 |
| 2642 | Seq. ID No. 5533 | Seq. ID No. 5534 |
| 2648a | Seq. ID No. 5543 | Seq. ID No. 5544 |
| 2648b | Seq. ID No. 5545 | Seq. ID No. 5546 |
| 2650 | Seq. ID No. 5549 | Seq. ID No. 5550 |
| 2660 | Seq. ID No. 5569 | Seq. ID No. 5570 |
| 2665 | Seq. ID No. 5577 | Seq. ID No. 5578 |
| 2672 | Seq. ID No. 5593 | Seq. ID No. 5594 |
| 2673 | Seq. ID No. 5595 | Seq. ID No. 5596 |
| 2679a | Seq. ID No. 5609 | Seq. ID No. 5610 |
| 2683a | Seq. ID No. 5623 | Seq. ID No. 5624 |
| 2683b | Seq. ID No. 5625 | Seq. ID No. 5626 |
| 2689a | Seq. ID No. 5639 | Seq. ID No. 5640 |
| 2689b | Seq. ID No. 5641 | Seq. ID No. 5642 |
| 2690a | Seq. ID No. 5643 | Seq. ID No. 5644 |
| 2692b | Seq. ID No. 5649 | Seq. ID No. 5650 |
| 2696a | Seq. ID No. 5659 | Seq. ID No. 5660 |
| 2696b | Seq. ID No. 5661 | Seq. ID No. 5662 |
| 2705 | Seq. ID No. 5663 | Seq. ID No. 5664 |
| 2710c | Seq. ID No. 5667 | Seq. ID No. 5668 |
| 2713 | Seq. ID No. 5671 | Seq. ID No. 5672 |
| 2719a | Seq. ID No. 5687 | Seq. ID No. 5688 |
| 2719b | Seq. ID No. 5689 | Seq. ID No. 5690 |
| 2725a | Seq. ID No. 5703 | Seq. ID No. 5704 |
| 2725b | Seq. ID No. 5705 | Seq. ID No. 5706 |
| 2728a | Seq. ID No. 5709 | Seq. ID No. 5710 |
| 2728b | Seq. ID No. 5711 | Seq. ID No. 5712 |
| 2742a | Seq. ID No. 5737 | Seq. ID No. 5738 |
| 2742b | Seq. ID No. 5739 | Seq. ID No. 5740 |
| 2744 | Seq. ID No. 5745 | Seq. ID No. 5746 |
| 2746 | Seq. ID No. 5751 | Seq. ID No. 5752 |
| 2749a | Seq. ID No. 5763 | Seq. ID No. 5764 |
| 2760a | Seq. ID No. 5797 | Seq. ID No. 5798 |
| 2764 | Seq. ID No. 5811 | Seq. ID No. 5812 |
| 2765b | Seq. ID No. 5813 | Seq. ID No. 5814 |
| 2765c | Seq. ID No. 5815 | Seq. ID No. 5816 |
| 2769a | Seq. ID No. 5827 | Seq. ID No. 5828 |
| 2770 | Seq. ID No. 5829 | Seq. ID No. 5830 |
| 2776a | Seq. ID No. 5843 | Seq. ID No. 5844 |
| 2776b | Seq. ID No. 5845 | Seq. ID No. 5846 |
| 2788b | Seq. ID No. 5873 | Seq. ID No. 5874 |
| 2791 | Seq. ID No. 5879 | Seq. ID No. 5880 |
| 2794 | Seq. ID No. 5883 | Seq. ID No. 5884 |
| 2796a | Seq. ID No. 5885 | Seq. ID No. 5886 |
| 2798 | Seq. ID No. 5887 | Seq. ID No. 5888 |
| 2799a | Seq. ID No. 5889 | Seq. ID No. 5890 |
| 2799b | Seq. ID No. 5891 | Seq. ID No. 5892 |
| 2819 | Seq. ID No. 5931 | Seq. ID No. 5932 |
| 2832 | Seq. ID No. 5955 | Seq. ID No. 5956 |
| 2837a | Seq. ID No. 5963 | Seq. ID No. 5964 |
| 2837b | Seq. ID No. 5965 | Seq. ID No. 5966 |
| 2845 | Seq. ID No. 5975 | Seq. ID No. 5976 |
| 2850 | Seq. ID No. 5983 | Seq. ID No. 5984 |
| 2851 | Seq. ID No. 5985 | Seq. ID No. 5986 |
| 2852 | Seq. ID No. 5987 | Seq. ID No. 5988 |
| 2853 | Seq. ID No. 5989 | Seq. ID No. 5990 |
| 2859 | Seq. ID No. 6005 | Seq. ID No. 6006 |
| 2862 | Seq. ID No. 6013 | Seq. ID No. 6014 |
| 2872 | Seq. ID No. 6031 | Seq. ID No. 6032 |
| 2877 | Seq. ID No. 6035 | Seq. ID No. 6036 |
| 2882 | Seq. ID No. 6045 | Seq. ID No. 6046 |
| 2887a | Seq. ID No. 6059 | Seq. ID No. 6060 |
| 2887b | Seq. ID No. 6061 | Seq. ID No. 6062 |
| 2888 | Seq. ID No. 6063 | Seq. ID No. 6064 |
| 2893b | Seq. ID No. 6073 | Seq. ID No. 6074 |
| 2895 | Seq. ID No. 6081 | Seq. ID No. 6082 |
| 2901a | Seq. ID No. 6091 | Seq. ID No. 6092 |
| 2901b | Seq. ID No. 6093 | Seq. ID No. 6094 |
| 2917 | Seq. ID No. 6127 | Seq. ID No. 6128 |
| 2929b | Seq. ID No. 6159 | Seq. ID No. 6160 |
| 2944b | Seq. ID No. 6197 | Seq. ID No. 6198 |
| 2945b | Seq. ID No. 6199 | Seq. ID No. 6200 |
| 2948a | Seq. ID No. 6203 | Seq. ID No. 6204 |
| 2952 | Seq. ID No. 6217 | Seq. ID No. 6218 |
| 2955a | Seq. ID No. 6221 | Seq. ID No. 6222 |
| 2955b | Seq. ID No. 6223 | Seq. ID No. 6224 |
| 2958a | Seq. ID No. 6237 | Seq. ID No. 6238 |
| 2958b | Seq. ID No. 6239 | Seq. ID No. 6240 |
| 2958c | Seq. ID No. 6241 | Seq. ID No. 6242 |
| 2960a | Seq. ID No. 6245 | Seq. ID No. 6246 |
| 2962a | Seq. ID No. 6247 | Seq. ID No. 6248 |
| 2962b | Seq. ID No. 6249 | Seq. ID No. 6250 |
| 2966 | Seq. ID No. 6259 | Seq. ID No. 6260 |
| 2970a | Seq. ID No. 6265 | Seq. ID No. 6266 |
| 2970b | Seq. ID No. 6267 | Seq. ID No. 6268 |
| 2974a | Seq. ID No. 6273 | Seq. ID No. 6274 |
| 2978a | Seq. ID No. 6283 | Seq. ID No. 6284 |
| 2978b | Seq. ID No. 6285 | Seq. ID No. 6286 |
| 2992 | Seq. ID No. 6323 | Seq. ID No. 6324 |
| 2993a | Seq. ID No. 6325 | Seq. ID No. 6326 |
| 2993b | Seq. ID No. 6327 | Seq. ID No. 6328 |
| 3015 | Seq. ID No. 6361 | Seq. ID No. 6362 |
| 3016a | Seq. ID No. 6363 | Seq. ID No. 6364 |
| 3021 | Seq. ID No. 6377 | Seq. ID No. 6378 |
| 3023 | Seq. ID No. 6381 | Seq. ID No. 6382 |
| 3031a | Seq. ID No. 6401 | Seq. ID No. 6402 |
| 3031b | Seq. ID No. 6403 | Seq. ID No. 6404 |
| 3038 | Seq. ID No. 6419 | Seq. ID No. 6420 |
| 3042a | Seq. ID No. 6425 | Seq. ID No. 6426 |
| 3042b | Seq. ID No. 6427 | Seq. ID No. 6428 |
| 3043a | Seq. ID No. 6429 | Seq. ID No. 6430 |
| 3043b | Seq. ID No. 6431 | Seq. ID No. 6432 |
| 3044 | Seq. ID No. 6433 | Seq. ID No. 6434 |
| 3045a | Seq. ID No. 6435 | Seq. ID No. 6436 |
| 3045b | Seq. ID No. 6437 | Seq. ID No. 6438 |
| 3048a | Seq. ID No. 6439 | Seq. ID No. 6440 |
| 3051a | Seq. ID No. 6447 | Seq. ID No. 6448 |
| 3051b | Seq. ID No. 6449 | Seq. ID No. 6450 |
| 3052 | Seq. ID No. 6451 | Seq. ID No. 6452 |
| 3054 | Seq. ID No. 6453 | Seq. ID No. 6454 |
| 3055a | Seq. ID No. 6455 | Seq. ID No. 6456 |
| 3055b | Seq. ID No. 6457 | Seq. ID No. 6458 |
| 3061 | Seq. ID No. 6465 | Seq. ID No. 6466 |
| 3062 | Seq. ID No. 6467 | Seq. ID No. 6468 |
| 3063a | Seq. ID No. 6469 | Seq. ID No. 6470 |
| 3063b | Seq. ID No. 6471 | Seq. ID No. 6472 |
| 3064b | Seq. ID No. 6473 | Seq. ID No. 6474 |
| 3065a | Seq. ID No. 6475 | Seq. ID No. 6476 |
| 3065b | Seq. ID No. 6477 | Seq. ID No. 6478 |
| 3066 | Seq. ID No. 6479 | Seq. ID No. 6480 |
| 3067 | Seq. ID No. 6481 | Seq. ID No. 6482 |
| 3068 | Seq. ID No. 6483 | Seq. ID No. 6484 |
| 3069 | Seq. ID No. 6485 | Seq. ID No. 6486 |
| 3071 | Seq. ID No. 6487 | Seq. ID No. 6488 |
| 3072a | Seq. ID No. 6489 | Seq. ID No. 6490 |
| 3073 | Seq. ID No. 6491 | Seq. ID No. 6492 |
| 3075 | Seq. ID No. 6495 | Seq. ID No. 6496 |
| 3076 | Seq. ID No. 6497 | Seq. ID No. 6498 |
| 3079 | Seq. ID No. 6507 | Seq. ID No. 6508 |
| 3085 | Seq. ID No. 6517 | Seq. ID No. 6518 |
| 3086 | Seq. ID No. 6519 | Seq. ID No. 6520 |
| 3087a | Seq. ID No. 6521 | Seq. ID No. 6522 |
| 3087c | Seq. ID No. 6523 | Seq. ID No. 6524 |
| 3088 | Seq. ID No. 6525 | Seq. ID No. 6526 |
| 3089a | Seq. ID No. 6527 | Seq. ID No. 6528 |
| 3089b | Seq. ID No. 6529 | Seq. ID No. 6530 |
| 3091 | Seq. ID No. 6533 | Seq. ID No. 6534 |
| 3095 | Seq. ID No. 6539 | Seq. ID No. 6540 |
| 3096a | Seq. ID No. 6541 | Seq. ID No. 6542 |
| 3096b | Seq. ID No. 6543 | Seq. ID No. 6544 |
| 3100a | Seq. ID No. 6555 | Seq. ID No. 6556 |
| 3100b | Seq. ID No. 6557 | Seq. ID No. 6558 |
| 3102 | Seq. ID No. 6561 | Seq. ID No. 6562 |
| 3103b | Seq. ID No. 6563 | Seq. ID No. 6564 |
| 3104 | Seq. ID No. 6565 | Seq. ID No. 6566 |
| 3105 | Seq. ID No. 6567 | Seq. ID No. 6568 |
| 3106 | Seq. ID No. 6569 | Seq. ID No. 6570 |
| 3107a | Seq. ID No. 6571 | Seq. ID No. 6572 |
| 3107b | Seq. ID No. 6573 | Seq. ID No. 6574 |
| 3110a | Seq. ID No. 6581 | Seq. ID No. 6582 |
| 3110b | Seq. ID No. 6583 | Seq. ID No. 6584 |
| 3140 | Seq. ID No. 6631 | Seq. ID No. 6632 |
| 3145a | Seq. ID No. 6635 | Seq. ID No. 6636 |
| 3145b | Seq. ID No. 6637 | Seq. ID No. 6638 |
| 3145c | Seq. ID No. 6639 | Seq. ID No. 6640 |
| 3147b | Seq. ID No. 6643 | Seq. ID No. 6644 |
| 3147c | Seq. ID No. 6645 | Seq. ID No. 6646 |
| 3149a | Seq. ID No. 6647 | Seq. ID No. 6648 |
| 3150a | Seq. ID No. 6649 | Seq. ID No. 6650 |

Listed in Table 18 are 1529 ORFs detected by the Glimmer™ ORF finder program.

TABLE 18
GLIMMER ™ ORFs

| ORF Number | DNA SEQ ID Number | Protein SEQ ID Number |
|---|---|---|
| 1 | Seq. ID No. 1 | Seq. ID No. 2 |
| 2b | Seq. ID No. 5 | Seq. ID No. 6 |
| 4c | Seq. ID No. 11 | Seq. ID No. 12 |
| 5 | Seq. ID No. 13 | Seq. ID No. 14 |
| 7 | Seq. ID No. 15 | Seq. ID No. 16 |
| 10 | Seq. ID No. 17 | Seq. ID No. 18 |
| 17 | Seq. ID No. 25 | Seq. ID No. 26 |
| 18 | Seq. ID No. 27 | Seq. ID No. 28 |
| 20 | Seq. ID No. 29 | Seq. ID No. 30 |
| 23b | Seq. ID No. 35 | Seq. ID No. 36 |
| 24 | Seq. ID No. 37 | Seq. ID No. 38 |
| 36 | Seq. ID No. 41 | Seq. ID No. 42 |
| 37 | Seq. ID No. 43 | Seq. ID No. 44 |
| 38b | Seq. ID No. 47 | Seq. ID No. 48 |
| 39b | Seq. ID No. 51 | Seq. ID No. 52 |
| 40 | Seq. ID No. 53 | Seq. ID No. 54 |
| 41c | Seq. ID No. 59 | Seq. ID No. 60 |
| 43c | Seq. ID No. 63 | Seq. ID No. 64 |
| 45 | Seq. ID No. 69 | Seq. ID No. 70 |
| 46b | Seq. ID No. 73 | Seq. ID No. 74 |
| 47 | Seq. ID No. 75 | Seq. ID No. 76 |
| 48c | Seq. ID No. 81 | Seq. ID No. 82 |
| 50 | Seq. ID No. 83 | Seq. ID No. 84 |
| 51c | Seq. ID No. 89 | Seq. ID No. 90 |
| 56b | Seq. ID No. 93 | Seq. ID No. 94 |
| 57b | Seq. ID No. 97 | Seq. ID No. 98 |
| 60b | Seq. ID No. 103 | Seq. ID No. 104 |
| 62c | Seq. ID No. 111 | Seq. ID No. 112 |
| 65c | Seq. ID No. 121 | Seq. ID No. 122 |
| 67 | Seq. ID No. 125 | Seq. ID No. 126 |
| 71 | Seq. ID No. 129 | Seq. ID No. 130 |
| 74 | Seq. ID No. 135 | Seq. ID No. 136 |
| 77 | Seq. ID No. 137 | Seq. ID No. 138 |
| 80c | Seq. ID No. 145 | Seq. ID No. 146 |
| 81b | Seq. ID No. 149 | Seq. ID No. 150 |
| 85c | Seq. ID No. 157 | Seq. ID No. 158 |
| 88 | Seq. ID No. 167 | Seq. ID No. 168 |
| 91b | Seq. ID No. 173 | Seq. ID No. 174 |
| 93c | Seq. ID No. 183 | Seq. ID No. 184 |
| 95b | Seq. ID No. 187 | Seq. ID No. 188 |
| 97b | Seq. ID No. 189 | Seq. ID No. 190 |
| 99 | Seq. ID No. 191 | Seq. ID No. 192 |
| 102 | Seq. ID No. 195 | Seq. ID No. 196 |
| 104a | Seq. ID No. 197 | Seq. ID No. 198 |
| 105a | Seq. ID No. 203 | Seq. ID No. 204 |
| 106 | Seq. ID No. 207 | Seq. ID No. 208 |
| 109d | Seq. ID No. 215 | Seq. ID No. 216 |
| 111b | Seq. ID No. 219 | Seq. ID No. 220 |
| 112b | Seq. ID No. 223 | Seq. ID No. 224 |
| 114 | Seq. ID No. 225 | Seq. ID No. 226 |
| 115 | Seq. ID No. 227 | Seq. ID No. 228 |
| 119b | Seq. ID No. 231 | Seq. ID No. 232 |
| 121 | Seq. ID No. 233 | Seq. ID No. 234 |
| 124c | Seq. ID No. 239 | Seq. ID No. 240 |
| 125 | Seq. ID No. 241 | Seq. ID No. 242 |
| 127c | Seq. ID No. 247 | Seq. ID No. 248 |
| 129c | Seq. ID No. 253 | Seq. ID No. 254 |
| 134 | Seq. ID No. 261 | Seq. ID No. 262 |
| 135b | Seq. ID No. 265 | Seq. ID No. 266 |
| 137b | Seq. ID No. 269 | Seq. ID No. 270 |
| 138b | Seq. ID No. 273 | Seq. ID No. 274 |
| 142 | Seq. ID No. 275 | Seq. ID No. 276 |
| 143 | Seq. ID No. 277 | Seq. ID No. 278 |
| 144c | Seq. ID No. 283 | Seq. ID No. 284 |
| 145d | Seq. ID No. 291 | Seq. ID No. 292 |
| 147 | Seq. ID No. 293 | Seq. ID No. 294 |
| 151b | Seq. ID No. 297 | Seq. ID No. 298 |
| 152b | Seq. ID No. 301 | Seq. ID No. 302 |
| 153c | Seq. ID No. 307 | Seq. ID No. 308 |
| 154 | Seq. ID No. 309 | Seq. ID No. 310 |
| 155b | Seq. ID No. 313 | Seq. ID No. 314 |
| 157b | Seq. ID No. 317 | Seq. ID No. 318 |
| 158b | Seq. ID No. 321 | Seq. ID No. 322 |
| 160b | Seq. ID No. 325 | Seq. ID No. 326 |
| 162e | Seq. ID No. 335 | Seq. ID No. 336 |
| 165b | Seq. ID No. 343 | Seq. ID No. 344 |
| 167 | Seq. ID No. 345 | Seq. ID No. 346 |
| 171b | Seq. ID No. 353 | Seq. ID No. 354 |
| 172 | Seq. ID No. 355 | Seq. ID No. 356 |
| 175 | Seq. ID No. 361 | Seq. ID No. 362 |
| 176 | Seq. ID No. 363 | Seq. ID No. 364 |
| 177b | Seq. ID No. 367 | Seq. ID No. 368 |
| 180b | Seq. ID No. 375 | Seq. ID No. 376 |
| 184 | Seq. ID No. 379 | Seq. ID No. 380 |
| 186 | Seq. ID No. 383 | Seq. ID No. 384 |
| 188 | Seq. ID No. 389 | Seq. ID No. 390 |
| 189b | Seq. ID No. 391 | Seq. ID No. 392 |
| 192b | Seq. ID No. 397 | Seq. ID No. 398 |
| 195 | Seq. ID No. 405 | Seq. ID No. 406 |
| 197b | Seq. ID No. 411 | Seq. ID No. 412 |
| 198b | Seq. ID No. 415 | Seq. ID No. 416 |
| 200 | Seq. ID No. 417 | Seq. ID No. 418 |
| 203 | Seq. ID No. 423 | Seq. ID No. 424 |
| 204 | Seq. ID No. 425 | Seq. ID No. 426 |
| 205 | Seq. ID No. 427 | Seq. ID No. 428 |
| 206 | Seq. ID No. 429 | Seq. ID No. 430 |
| 208b | Seq. ID No. 435 | Seq. ID No. 436 |
| 210 | Seq. ID No. 437 | Seq. ID No. 438 |
| 211 | Seq. ID No. 439 | Seq. ID No. 440 |
| 213 | Seq. ID No. 441 | Seq. ID No. 442 |
| 218 | Seq. ID No. 445 | Seq. ID No. 446 |
| 219 | Seq. ID No. 447 | Seq. ID No. 448 |
| 220c | Seq. ID No. 453 | Seq. ID No. 454 |
| 226b | Seq. ID No. 461 | Seq. ID No. 462 |
| 227 | Seq. ID No. 463 | Seq. ID No. 464 |
| 228d | Seq. ID No. 471 | Seq. ID No. 472 |
| 236c | Seq. ID No. 485 | Seq. ID No. 486 |
| 245c | Seq. ID No. 501 | Seq. ID No. 502 |
| 246b | Seq. ID No. 505 | Seq. ID No. 506 |
| 248b | Seq. ID No. 511 | Seq. ID No. 512 |
| 249b | Seq. ID No. 515 | Seq. ID No. 516 |
| 251 | Seq. ID No. 517 | Seq. ID No. 518 |
| 253b | Seq. ID No. 521 | Seq. ID No. 522 |
| 254 | Seq. ID No. 523 | Seq. ID No. 524 |
| 255d | Seq. ID No. 531 | Seq. ID No. 532 |
| 258 | Seq. ID No. 533 | Seq. ID No. 534 |
| 259c | Seq. ID No. 539 | Seq. ID No. 540 |
| 260 | Seq. ID No. 541 | Seq. ID No. 542 |
| 262 | Seq. ID No. 543 | Seq. ID No. 544 |
| 265 | Seq. ID No. 551 | Seq. ID No. 552 |
| 267c | Seq. ID No. 555 | Seq. ID No. 556 |
| 269b | Seq. ID No. 561 | Seq. ID No. 562 |
| 271b | Seq. ID No. 565 | Seq. ID No. 566 |
| 273b | Seq. ID No. 569 | Seq. ID No. 570 |
| 277c | Seq. ID No. 575 | Seq. ID No. 576 |
| 279b | Seq. ID No. 579 | Seq. ID No. 580 |
| 280d | Seq. ID No. 587 | Seq. ID No. 588 |
| 283b | Seq. ID No. 593 | Seq. ID No. 594 |
| 284b | Seq. ID No. 597 | Seq. ID No. 598 |
| 286b | Seq. ID No. 603 | Seq. ID No. 604 |
| 287 | Seq. ID No. 605 | Seq. ID No. 606 |
| 290b | Seq. ID No. 609 | Seq. ID No. 610 |
| 293 | Seq. ID No. 611 | Seq. ID No. 612 |
| 294 | Seq. ID No. 613 | Seq. ID No. 614 |
| 296 | Seq. ID No. 615 | Seq. ID No. 616 |
| 301b | Seq. ID No. 623 | Seq. ID No. 624 |
| 304b | Seq. ID No. 627 | Seq. ID No. 628 |
| 307 | Seq. ID No. 629 | Seq. ID No. 630 |
| 308 | Seq. ID No. 631 | Seq. ID No. 632 |
| 310 | Seq. ID No. 635 | Seq. ID No. 636 |
| 311 | Seq. ID No. 637 | Seq. ID No. 638 |
| 313e | Seq. ID No. 647 | Seq. ID No. 648 |
| 315 | Seq. ID No. 649 | Seq. ID No. 650 |
| 318c | Seq. ID No. 661 | Seq. ID No. 662 |
| 321 | Seq. ID No. 669 | Seq. ID No. 670 |
| 323b | Seq. ID No. 673 | Seq. ID No. 674 |
| 324 | Seq. ID No. 675 | Seq. ID No. 676 |
| 327c | Seq. ID No. 681 | Seq. ID No. 682 |
| 329 | Seq. ID No. 683 | Seq. ID No. 684 |
| 330c | Seq. ID No. 689 | Seq. ID No. 690 |
| 335b | Seq. ID No. 701 | Seq. ID No. 702 |
| 337 | Seq. ID No. 703 | Seq. ID No. 704 |
| 338c | Seq. ID No. 709 | Seq. ID No. 710 |
| 341b | Seq. ID No. 713 | Seq. ID No. 714 |
| 342h | Seq. ID No. 729 | Seq. ID No. 730 |
| 344b | Seq. ID No. 733 | Seq. ID No. 734 |
| 345 | Seq. ID No. 735 | Seq. ID No. 736 |
| 346a | Seq. ID No. 737 | Seq. ID No. 738 |
| 347 | Seq. ID No. 741 | Seq. ID No. 742 |
| 349c | Seq. ID No. 747 | Seq. ID No. 748 |
| 350b | Seq. ID No. 751 | Seq. ID No. 752 |
| 351 | Seq. ID No. 753 | Seq. ID No. 754 |
| 352c | Seq. ID No. 759 | Seq. ID No. 760 |
| 354c | Seq. ID No. 765 | Seq. ID No. 766 |
| 355 | Seq. ID No. 767 | Seq. ID No. 768 |
| 356b | Seq. ID No. 771 | Seq. ID No. 772 |
| 358c | Seq. ID No. 781 | Seq. ID No. 782 |
| 362 | Seq. ID No. 783 | Seq. ID No. 784 |
| 365b | Seq. ID No. 787 | Seq. ID No. 788 |
| 366c | Seq. ID No. 791 | Seq. ID No. 792 |
| 367b | Seq. ID No. 795 | Seq. ID No. 796 |
| 371b | Seq. ID No. 799 | Seq. ID No. 800 |
| 372d | Seq. ID No. 807 | Seq. ID No. 808 |
| 378b | Seq. ID No. 817 | Seq. ID No. 818 |
| 382b | Seq. ID No. 819 | Seq. ID No. 820 |
| 383b | Seq. ID No. 823 | Seq. ID No. 824 |
| 384b | Seq. ID No. 827 | Seq. ID No. 828 |
| 385b | Seq. ID No. 831 | Seq. ID No. 832 |
| 387 | Seq. ID No. 835 | Seq. ID No. 836 |
| 390 | Seq. ID No. 837 | Seq. ID No. 838 |
| 391 | Seq. ID No. 839 | Seq. ID No. 840 |
| 395c | Seq. ID No. 847 | Seq. ID No. 848 |
| 398 | Seq. ID No. 851 | Seq. ID No. 852 |
| 400b | Seq. ID No. 855 | Seq. ID No. 856 |
| 401b | Seq. ID No. 859 | Seq. ID No. 860 |
| 403d | Seq. ID No. 867 | Seq. ID No. 868 |
| 406b | Seq. ID No. 871 | Seq. ID No. 872 |
| 409b | Seq. ID No. 875 | Seq. ID No. 876 |
| 410c | Seq. ID No. 881 | Seq. ID No. 882 |
| 413b | Seq. ID No. 885 | Seq. ID No. 886 |
| 416b | Seq. ID No. 889 | Seq. ID No. 890 |
| 417 | Seq. ID No. 891 | Seq. ID No. 892 |
| 418b | Seq. ID No. 895 | Seq. ID No. 896 |
| 419c | Seq. ID No. 901 | Seq. ID No. 902 |
| 421 | Seq. ID No. 907 | Seq. ID No. 908 |
| 424c | Seq. ID No. 915 | Seq. ID No. 916 |
| 426b | Seq. ID No. 919 | Seq. ID No. 920 |
| 427c | Seq. ID No. 925 | Seq. ID No. 926 |
| 429b | Seq. ID No. 929 | Seq. ID No. 930 |
| 431 | Seq. ID No. 935 | Seq. ID No. 936 |
| 435 | Seq. ID No. 939 | Seq. ID No. 940 |
| 438c | Seq. ID No. 949 | Seq. ID No. 950 |
| 440 | Seq. ID No. 951 | Seq. ID No. 952 |
| 441b | Seq. ID No. 955 | Seq. ID No. 956 |
| 444b | Seq. ID No. 961 | Seq. ID No. 962 |
| 445b | Seq. ID No. 965 | Seq. ID No. 966 |
| 448 | Seq. ID No. 967 | Seq. ID No. 968 |
| 450b | Seq. ID No. 971 | Seq. ID No. 972 |
| 452e | Seq. ID No. 985 | Seq. ID No. 986 |
| 455c | Seq. ID No. 993 | Seq. ID No. 994 |
| 458b | Seq. ID No. 997 | Seq. ID No. 998 |
| 460c | Seq. ID No. 1003 | Seq. ID No. 1004 |
| 462 | Seq. ID No. 1005 | Seq. ID No. 1006 |
| 463b | Seq. ID No. 1009 | Seq. ID No. 1010 |
| 464d | Seq. ID No. 1017 | Seq. ID No. 1018 |
| 467b | Seq. ID No. 1023 | Seq. ID No. 1024 |
| 472f | Seq. ID No. 1039 | Seq. ID No. 1040 |
| 473 | Seq. ID No. 1041 | Seq. ID No. 1042 |
| 474 | Seq. ID No. 1043 | Seq. ID No. 1044 |
| 478b | Seq. ID No. 1051 | Seq. ID No. 1052 |
| 479 | Seq. ID No. 1053 | Seq. ID No. 1054 |
| 480b | Seq. ID No. 1057 | Seq. ID No. 1058 |
| 481b | Seq. ID No. 1061 | Seq. ID No. 1062 |
| 482b | Seq. ID No. 1065 | Seq. ID No. 1066 |
| 483 | Seq. ID No. 1067 | Seq. ID No. 1068 |
| 490 | Seq. ID No. 1069 | Seq. ID No. 1070 |
| 491b | Seq. ID No. 1073 | Seq. ID No. 1074 |
| 492 | Seq. ID No. 1075 | Seq. ID No. 1076 |
| 495c | Seq. ID No. 1083 | Seq. ID No. 1084 |
| 498b | Seq. ID No. 1087 | Seq. ID No. 1088 |
| 499b | Seq. ID No. 1091 | Seq. ID No. 1092 |
| 500 | Seq. ID No. 1093 | Seq. ID No. 1094 |
| 502c | Seq. ID No. 1097 | Seq. ID No. 1098 |
| 504 | Seq. ID No. 1099 | Seq. ID No. 1100 |
| 505b | Seq. ID No. 1103 | Seq. ID No. 1104 |
| 509 | Seq. ID No. 1109 | Seq. ID No. 1110 |
| 512b | Seq. ID No. 1115 | Seq. ID No. 1116 |
| 514 | Seq. ID No. 1117 | Seq. ID No. 1118 |
| 515c | Seq. ID No. 1123 | Seq. ID No. 1124 |
| 518c | Seq. ID No. 1129 | Seq. ID No. 1130 |
| 523 | Seq. ID No. 1133 | Seq. ID No. 1134 |
| 524b | Seq. ID No. 1137 | Seq. ID No. 1138 |
| 527b | Seq. ID No. 1143 | Seq. ID No. 1144 |
| 528b | Seq. ID No. 1147 | Seq. ID No. 1148 |
| 529 | Seq. ID No. 1149 | Seq. ID No. 1150 |
| 533 | Seq. ID No. 1155 | Seq. ID No. 1156 |
| 535 | Seq. ID No. 1157 | Seq. ID No. 1158 |
| 536 | Seq. ID No. 1159 | Seq. ID No. 1160 |
| 539c | Seq. ID No. 1163 | Seq. ID No. 1164 |
| 541d | Seq. ID No. 1171 | Seq. ID No. 1172 |
| 543c | Seq. ID No. 1179 | Seq. ID No. 1180 |
| 544 | Seq. ID No. 1181 | Seq. ID No. 1182 |
| 545c | Seq. ID No. 1187 | Seq. ID No. 1188 |
| 546c | Seq. ID No. 1193 | Seq. ID No. 1194 |
| 548 | Seq. ID No. 1199 | Seq. ID No. 1200 |
| 551 | Seq. ID No. 1205 | Seq. ID No. 1206 |
| 554b | Seq. ID No. 1211 | Seq. ID No. 1212 |
| 557b | Seq. ID No. 1217 | Seq. ID No. 1218 |
| 560b | Seq. ID No. 1221 | Seq. ID No. 1222 |
| 561 | Seq. ID No. 1223 | Seq. ID No. 1224 |
| 563 | Seq. ID No. 1225 | Seq. ID No. 1226 |
| 564c | Seq. ID No. 1231 | Seq. ID No. 1232 |
| 569 | Seq. ID No. 1233 | Seq. ID No. 1234 |
| 570b | Seq. ID No. 1237 | Seq. ID No. 1238 |
| 573 | Seq. ID No. 1241 | Seq. ID No. 1242 |
| 580b | Seq. ID No. 1253 | Seq. ID No. 1254 |
| 583b | Seq. ID No. 1257 | Seq. ID No. 1258 |
| 586b | Seq. ID No. 1263 | Seq. ID No. 1264 |
| 587d | Seq. ID No. 1271 | Seq. ID No. 1272 |
| 590 | Seq. ID No. 1281 | Seq. ID No. 1282 |
| 609b | Seq. ID No. 1317 | Seq. ID No. 1318 |
| 611 | Seq. ID No. 1323 | Seq. ID No. 1324 |
| 613d | Seq. ID No. 1331 | Seq. ID No. 1332 |
| 614b | Seq. ID No. 1335 | Seq. ID No. 1336 |
| 618 | Seq. ID No. 1339 | Seq. ID No. 1340 |
| 620 | Seq. ID No. 1345 | Seq. ID No. 1346 |
| 623 | Seq. ID No. 1347 | Seq. ID No. 1348 |
| 626c | Seq. ID No. 1361 | Seq. ID No. 1362 |
| 631d | Seq. ID No. 1369 | Seq. ID No. 1370 |
| 633c | Seq. ID No. 1377 | Seq. ID No. 1378 |
| 634b | Seq. ID No. 1379 | Seq. ID No. 1380 |
| 637c | Seq. ID No. 1387 | Seq. ID No. 1388 |
| 641b | Seq. ID No. 1393 | Seq. ID No. 1394 |
| 643 | Seq. ID No. 1395 | Seq. ID No. 1396 |
| 644b | Seq. ID No. 1399 | Seq. ID No. 1400 |
| 650 | Seq. ID No. 1403 | Seq. ID No. 1404 |
| 652 | Seq. ID No. 1405 | Seq. ID No. 1406 |
| 653c | Seq. ID No. 1411 | Seq. ID No. 1412 |
| 656 | Seq. ID No. 1413 | Seq. ID No. 1414 |
| 657c | Seq. ID No. 1415 | Seq. ID No. 1416 |
| 658b | Seq. ID No. 1419 | Seq. ID No. 1420 |
| 661 | Seq. ID No. 1425 | Seq. ID No. 1426 |
| 663 | Seq. ID No. 1427 | Seq. ID No. 1428 |
| 665 | Seq. ID No. 1429 | Seq. ID No. 1430 |
| 666 | Seq. ID No. 1431 | Seq. ID No. 1432 |
| 670 | Seq. ID No. 1433 | Seq. ID No. 1434 |
| 674b | Seq. ID No. 1437 | Seq. ID No. 1438 |
| 677b | Seq. ID No. 1443 | Seq. ID No. 1444 |
| 680 | Seq. ID No. 1449 | Seq. ID No. 1450 |
| 681 | Seq. ID No. 1451 | Seq. ID No. 1452 |
| 683b | Seq. ID No. 1455 | Seq. ID No. 1456 |
| 684 | Seq. ID No. 1457 | Seq. ID No. 1458 |
| 687 | Seq. ID No. 1459 | Seq. ID No. 1460 |
| 688b | Seq. ID No. 1463 | Seq. ID No. 1464 |
| 690 | Seq. ID No. 1465 | Seq. ID No. 1466 |
| 691b | Seq. ID No. 1469 | Seq. ID No. 1470 |
| 692 | Seq. ID No. 1471 | Seq. ID No. 1472 |
| 694 | Seq. ID No. 1473 | Seq. ID No. 1474 |
| 696 | Seq. ID No. 1475 | Seq. ID No. 1476 |
| 698 | Seq. ID No. 1477 | Seq. ID No. 1478 |
| 700c | Seq. ID No. 1485 | Seq. ID No. 1486 |
| 703 | Seq. ID No. 1487 | Seq. ID No. 1488 |
| 705b | Seq. ID No. 1491 | Seq. ID No. 1492 |
| 707 | Seq. ID No. 1493 | Seq. ID No. 1494 |
| 709 | Seq. ID No. 1495 | Seq. ID No. 1496 |
| 710 | Seq. ID No. 1497 | Seq. ID No. 1498 |
| 712b | Seq. ID No. 1501 | Seq. ID No. 1502 |
| 715b | Seq. ID No. 1505 | Seq. ID No. 1506 |
| 717 | Seq. ID No. 1507 | Seq. ID No. 1508 |
| 718a | Seq. ID No. 1509 | Seq. ID No. 1510 |
| 722c | Seq. ID No. 1517 | Seq. ID No. 1518 |
| 724d | Seq. ID No. 1525 | Seq. ID No. 1526 |
| 726 | Seq. ID No. 1527 | Seq. ID No. 1528 |
| 728b | Seq. ID No. 1531 | Seq. ID No. 1532 |
| 730d | Seq. ID No. 1539 | Seq. ID No. 1540 |
| 731b | Seq. ID No. 1541 | Seq. ID No. 1542 |
| 732 | Seq. ID No. 1543 | Seq. ID No. 1544 |
| 738c | Seq. ID No. 1549 | Seq. ID No. 1550 |
| 743b | Seq. ID No. 1561 | Seq. ID No. 1562 |
| 744 | Seq. ID No. 1563 | Seq. ID No. 1564 |
| 745b | Seq. ID No. 1567 | Seq. ID No. 1568 |
| 746b | Seq. ID No. 1571 | Seq. ID No. 1572 |
| 747 | Seq. ID No. 1573 | Seq. ID No. 1574 |
| 748b | Seq. ID No. 1577 | Seq. ID No. 1578 |
| 751 | Seq. ID No. 1581 | Seq. ID No. 1582 |
| 754d | Seq. ID No. 1589 | Seq. ID No. 1590 |
| 756b | Seq. ID No. 1593 | Seq. ID No. 1594 |
| 758 | Seq. ID No. 1595 | Seq. ID No. 1596 |
| 760 | Seq. ID No. 1597 | Seq. ID No. 1598 |
| 762c | Seq. ID No. 1603 | Seq. ID No. 1604 |
| 763b | Seq. ID No. 1607 | Seq. ID No. 1608 |
| 766 | Seq. ID No. 1611 | Seq. ID No. 1612 |
| 767 | Seq. ID No. 1613 | Seq. ID No. 1614 |
| 770c | Seq. ID No. 1619 | Seq. ID No. 1620 |
| 772b | Seq. ID No. 1623 | Seq. ID No. 1624 |
| 775 | Seq. ID No. 1627 | Seq. ID No. 1628 |
| 777c | Seq. ID No. 1631 | Seq. ID No. 1632 |
| 780b | Seq. ID No. 1637 | Seq. ID No. 1638 |
| 782b | Seq. ID No. 1639 | Seq. ID No. 1640 |
| 785 | Seq. ID No. 1641 | Seq. ID No. 1642 |
| 788b | Seq. ID No. 1647 | Seq. ID No. 1648 |
| 789c | Seq. ID No. 1653 | Seq. ID No. 1654 |
| 792 | Seq. ID No. 1657 | Seq. ID No. 1658 |
| 794d | Seq. ID No. 1665 | Seq. ID No. 1666 |
| 797 | Seq. ID No. 1669 | Seq. ID No. 1670 |
| 799f | Seq. ID No. 1681 | Seq. ID No. 1682 |
| 803b | Seq. ID No. 1685 | Seq. ID No. 1686 |
| 804b | Seq. ID No. 1689 | Seq. ID No. 1690 |
| 807 | Seq. ID No. 1691 | Seq. ID No. 1692 |
| 812 | Seq. ID No. 1697 | Seq. ID No. 1698 |
| 813 | Seq. ID No. 1699 | Seq. ID No. 1700 |
| 826 | Seq. ID No. 1707 | Seq. ID No. 1708 |
| 829b | Seq. ID No. 1711 | Seq. ID No. 1712 |
| 830 | Seq. ID No. 1713 | Seq. ID No. 1714 |
| 834c | Seq. ID No. 1719 | Seq. ID No. 1720 |
| 835b | Seq. ID No. 1723 | Seq. ID No. 1724 |
| 836c | Seq. ID No. 1729 | Seq. ID No. 1730 |
| 837c | Seq. ID No. 1735 | Seq. ID No. 1736 |
| 843 | Seq. ID No. 1737 | Seq. ID No. 1738 |
| 847b | Seq. ID No. 1741 | Seq. ID No. 1742 |
| 849b | Seq. ID No. 1747 | Seq. ID No. 1748 |
| 851b | Seq. ID No. 1751 | Seq. ID No. 1752 |
| 853c | Seq. ID No. 1757 | Seq. ID No. 1758 |
| 854 | Seq. ID No. 1759 | Seq. ID No. 1760 |
| 855 | Seq. ID No. 1761 | Seq. ID No. 1762 |
| 859d | Seq. ID No. 1771 | Seq. ID No. 1772 |
| 863 | Seq. ID No. 1777 | Seq. ID No. 1778 |
| 866b | Seq. ID No. 1785 | Seq. ID No. 1786 |
| 868b | Seq. ID No. 1789 | Seq. ID No. 1790 |
| 870 | Seq. ID No. 1791 | Seq. ID No. 1792 |
| 873c | Seq. ID No. 1797 | Seq. ID No. 1798 |
| 874b | Seq. ID No. 1801 | Seq. ID No. 1802 |
| 875 | Seq. ID No. 1803 | Seq. ID No. 1804 |
| 876 | Seq. ID No. 1805 | Seq. ID No. 1806 |
| 878c | Seq. ID No. 1811 | Seq. ID No. 1812 |
| 880c | Seq. ID No. 1817 | Seq. ID No. 1818 |
| 885 | Seq. ID No. 1819 | Seq. ID No. 1820 |
| 886 | Seq. ID No. 1821 | Seq. ID No. 1822 |
| 888 | Seq. ID No. 1825 | Seq. ID No. 1826 |
| 890b | Seq. ID No. 1829 | Seq. ID No. 1830 |
| 907b | Seq. ID No. 1837 | Seq. ID No. 1838 |
| 908c | Seq. ID No. 1843 | Seq. ID No. 1844 |
| 911 | Seq. ID No. 1845 | Seq. ID No. 1846 |
| 912 | Seq. ID No. 1847 | Seq. ID No. 1848 |
| 916 | Seq. ID No. 1849 | Seq. ID No. 1850 |
| 917 | Seq. ID No. 1851 | Seq. ID No. 1852 |
| 918c | Seq. ID No. 1855 | Seq. ID No. 1856 |
| 923 | Seq. ID No. 1859 | Seq. ID No. 1860 |
| 925b | Seq. ID No. 1863 | Seq. ID No. 1864 |
| 926 | Seq. ID No. 1865 | Seq. ID No. 1866 |
| 927 | Seq. ID No. 1867 | Seq. ID No. 1868 |
| 929b | Seq. ID No. 1871 | Seq. ID No. 1872 |
| 930 | Seq. ID No. 1873 | Seq. ID No. 1874 |
| 932 | Seq. ID No. 1877 | Seq. ID No. 1878 |
| 934d | Seq. ID No. 1885 | Seq. ID No. 1886 |
| 935 | Seq. ID No. 1887 | Seq. ID No. 1888 |
| 938 | Seq. ID No. 1889 | Seq. ID No. 1890 |
| 939c | Seq. ID No. 1895 | Seq. ID No. 1896 |
| 942 | Seq. ID No. 1897 | Seq. ID No. 1898 |
| 943 | Seq. ID No. 1899 | Seq. ID No. 1900 |
| 944b | Seq. ID No. 1903 | Seq. ID No. 1904 |
| 946 | Seq. ID No. 1905 | Seq. ID No. 1906 |
| 949d | Seq. ID No. 1913 | Seq. ID No. 1914 |
| 951b | Seq. ID No. 1919 | Seq. ID No. 1920 |
| 952b | Seq. ID No. 1923 | Seq. ID No. 1924 |
| 954 | Seq. ID No. 1925 | Seq. ID No. 1926 |
| 956 | Seq. ID No. 1931 | Seq. ID No. 1932 |
| 958b | Seq. ID No. 1935 | Seq. ID No. 1936 |
| 959 | Seq. ID No. 1937 | Seq. ID No. 1938 |
| 961c | Seq. ID No. 1943 | Seq. ID No. 1944 |
| 963 | Seq. ID No. 1945 | Seq. ID No. 1946 |
| 965b | Seq. ID No. 1949 | Seq. ID No. 1950 |
| 966b | Seq. ID No. 1953 | Seq. ID No. 1954 |
| 967b | Seq. ID No. 1957 | Seq. ID No. 1958 |
| 968 | Seq. ID No. 1959 | Seq. ID No. 1960 |
| 969 | Seq. ID No. 1961 | Seq. ID No. 1962 |
| 972b | Seq. ID No. 1965 | Seq. ID No. 1966 |
| 976 | Seq. ID No. 1967 | Seq. ID No. 1968 |
| 977b | Seq. ID No. 1971 | Seq. ID No. 1972 |
| 978b | Seq. ID No. 1975 | Seq. ID No. 1976 |
| 980 | Seq. ID No. 1979 | Seq. ID No. 1980 |
| 982 | Seq. ID No. 1983 | Seq. ID No. 1984 |
| 984b | Seq. ID No. 1987 | Seq. ID No. 1988 |
| 986 | Seq. ID No. 1989 | Seq. ID No. 1990 |
| 987 | Seq. ID No. 1991 | Seq. ID No. 1992 |
| 988d | Seq. ID No. 1999 | Seq. ID No. 2000 |
| 991 | Seq. ID No. 2001 | Seq. ID No. 2002 |
| 992 | Seq. ID No. 2003 | Seq. ID No. 2004 |
| 995 | Seq. ID No. 2009 | Seq. ID No. 2010 |
| 998b | Seq. ID No. 2015 | Seq. ID No. 2016 |
| 999b | Seq. ID No. 2019 | Seq. ID No. 2020 |
| 1000 | Seq. ID No. 2021 | Seq. ID No. 2022 |
| 1004b | Seq. ID No. 2031 | Seq. ID No. 2032 |
| 1006b | Seq. ID No. 2035 | Seq. ID No. 2036 |
| 1007 | Seq. ID No. 2037 | Seq. ID No. 2038 |
| 1013c | Seq. ID No. 2045 | Seq. ID No. 2046 |
| 1016c | Seq. ID No. 2047 | Seq. ID No. 2048 |
| 1017d | Seq. ID No. 2055 | Seq. ID No. 2056 |
| 1018b | Seq. ID No. 2059 | Seq. ID No. 2060 |
| 1021c | Seq. ID No. 2065 | Seq. ID No. 2066 |
| 1024 | Seq. ID No. 2067 | Seq. ID No. 2068 |
| 1025 | Seq. ID No. 2069 | Seq. ID No. 2070 |
| 1026 | Seq. ID No. 2071 | Seq. ID No. 2072 |
| 1029 | Seq. ID No. 2073 | Seq. ID No. 2074 |
| 1033c | Seq. ID No. 2079 | Seq. ID No. 2080 |
| 1036c | Seq. ID No. 2091 | Seq. ID No. 2092 |
| 1037 | Seq. ID No. 2093 | Seq. ID No. 2094 |
| 1039b | Seq. ID No. 2097 | Seq. ID No. 2098 |
| 1041 | Seq. ID No. 2099 | Seq. ID No. 2100 |
| 1042b | Seq. ID No. 2103 | Seq. ID No. 2104 |
| 1047b | Seq. ID No. 2115 | Seq. ID No. 2116 |
| 1049b | Seq. ID No. 2119 | Seq. ID No. 2120 |
| 1050d | Seq. ID No. 2127 | Seq. ID No. 2128 |
| 1057 | Seq. ID No. 2135 | Seq. ID No. 2136 |
| 1058 | Seq. ID No. 2137 | Seq. ID No. 2138 |
| 1060b | Seq. ID No. 2141 | Seq. ID No. 2142 |
| 1062 | Seq. ID No. 2143 | Seq. ID No. 2144 |
| 1064 | Seq. ID No. 2147 | Seq. ID No. 2148 |
| 1065c | Seq. ID No. 2153 | Seq. ID No. 2154 |
| 1067 | Seq. ID No. 2159 | Seq. ID No. 2160 |
| 1069 | Seq. ID No. 2161 | Seq. ID No. 2162 |
| 1070b | Seq. ID No. 2165 | Seq. ID No. 2166 |
| 1076b | Seq. ID No. 2169 | Seq. ID No. 2170 |
| 1077e | Seq. ID No. 2179 | Seq. ID No. 2180 |
| 1079b | Seq. ID No. 2183 | Seq. ID No. 2184 |
| 1080a | Seq. ID No. 2185 | Seq. ID No. 2186 |
| 1081b | Seq. ID No. 2189 | Seq. ID No. 2190 |
| 1082 | Seq. ID No. 2191 | Seq. ID No. 2192 |
| 1085 | Seq. ID No. 2193 | Seq. ID No. 2194 |
| 1086 | Seq. ID No. 2195 | Seq. ID No. 2196 |
| 1087b | Seq. ID No. 2199 | Seq. ID No. 2200 |
| 1094b | Seq. ID No. 2207 | Seq. ID No. 2208 |
| 1096c | Seq. ID No. 2217 | Seq. ID No. 2218 |
| 1097 | Seq. ID No. 2219 | Seq. ID No. 2220 |
| 1098b | Seq. ID No. 2221 | Seq. ID No. 2222 |
| 1100 | Seq. ID No. 2223 | Seq. ID No. 2224 |
| 1101b | Seq. ID No. 2227 | Seq. ID No. 2228 |
| 1104 | Seq. ID No. 2229 | Seq. ID No. 2230 |
| 1106 | Seq. ID No. 2231 | Seq. ID No. 2232 |
| 1108a | Seq. ID No. 2233 | Seq. ID No. 2234 |
| 1111c | Seq. ID No. 2241 | Seq. ID No. 2242 |
| 1125 | Seq. ID No. 2245 | Seq. ID No. 2246 |
| 1126c | Seq. ID No. 2251 | Seq. ID No. 2252 |
| 1127 | Seq. ID No. 2253 | Seq. ID No. 2254 |
| 1131 | Seq. ID No. 2257 | Seq. ID No. 2258 |
| 1132 | Seq. ID No. 2259 | Seq. ID No. 2260 |
| 1135b | Seq. ID No. 2263 | Seq. ID No. 2264 |
| 1136b | Seq. ID No. 2267 | Seq. ID No. 2268 |
| 1138 | Seq. ID No. 2269 | Seq. ID No. 2270 |
| 1140c | Seq. ID No. 2275 | Seq. ID No. 2276 |
| 1142 | Seq. ID No. 2281 | Seq. ID No. 2282 |
| 1144c | Seq. ID No. 2287 | Seq. ID No. 2288 |
| 1146e | Seq. ID No. 2297 | Seq. ID No. 2298 |
| 1148 | Seq. ID No. 2299 | Seq. ID No. 2300 |
| 1149 | Seq. ID No. 2301 | Seq. ID No. 2302 |
| 1152g | Seq. ID No. 2315 | Seq. ID No. 2316 |
| 1155g | Seq. ID No. 2329 | Seq. ID No. 2330 |
| 1158b | Seq. ID No. 2333 | Seq. ID No. 2334 |
| 1160 | Seq. ID No. 2335 | Seq. ID No. 2336 |
| 1161b | Seq. ID No. 2339 | Seq. ID No. 2340 |
| 1165 | Seq. ID No. 2343 | Seq. ID No. 2344 |
| 1166 | Seq. ID No. 2345 | Seq. ID No. 2346 |
| 1167b | Seq. ID No. 2349 | Seq. ID No. 2350 |
| 1168 | Seq. ID No. 2351 | Seq. ID No. 2352 |
| 1169c | Seq. ID No. 2357 | Seq. ID No. 2358 |
| 1170 | Seq. ID No. 2359 | Seq. ID No. 2360 |
| 1171c | Seq. ID No. 2363 | Seq. ID No. 2364 |
| 1172 | Seq. ID No. 2365 | Seq. ID No. 2366 |
| 1174 | Seq. ID No. 2369 | Seq. ID No. 2370 |
| 1177 | Seq. ID No. 2373 | Seq. ID No. 2374 |
| 1179c | Seq. ID No. 2381 | Seq. ID No. 2382 |
| 1180e | Seq. ID No. 2391 | Seq. ID No. 2392 |
| 1183b | Seq. ID No. 2399 | Seq. ID No. 2400 |
| 1184 | Seq. ID No. 2401 | Seq. ID No. 2402 |
| 1185b | Seq. ID No. 2405 | Seq. ID No. 2406 |
| 1186b | Seq. ID No. 2409 | Seq. ID No. 2410 |
| 1188c | Seq. ID No. 2415 | Seq. ID No. 2416 |
| 1191 | Seq. ID No. 2417 | Seq. ID No. 2418 |
| 1192b | Seq. ID No. 2421 | Seq. ID No. 2422 |
| 1194 | Seq. ID No. 2423 | Seq. ID No. 2424 |
| 1196b | Seq. ID No. 2427 | Seq. ID No. 2428 |
| 1197 | Seq. ID No. 2429 | Seq. ID No. 2430 |
| 1198 | Seq. ID No. 2431 | Seq. ID No. 2432 |
| 1199b | Seq. ID No. 2435 | Seq. ID No. 2436 |
| 1200 | Seq. ID No. 2437 | Seq. ID No. 2438 |
| 1201 | Seq. ID No. 2439 | Seq. ID No. 2440 |
| 1202b | Seq. ID No. 2443 | Seq. ID No. 2444 |
| 1205 | Seq. ID No. 2445 | Seq. ID No. 2446 |
| 1207 | Seq. ID No. 2449 | Seq. ID No. 2450 |
| 1208 | Seq. ID No. 2451 | Seq. ID No. 2452 |
| 1209 | Seq. ID No. 2453 | Seq. ID No. 2454 |
| 1210b | Seq. ID No. 2457 | Seq. ID No. 2458 |
| 1213b | Seq. ID No. 2463 | Seq. ID No. 2464 |
| 1214 | Seq. ID No. 2465 | Seq. ID No. 2466 |
| 1215b | Seq. ID No. 2469 | Seq. ID No. 2470 |
| 1217c | Seq. ID No. 2471 | Seq. ID No. 2472 |
| 1218b | Seq. ID No. 2475 | Seq. ID No. 2476 |
| 1219 | Seq. ID No. 2477 | Seq. ID No. 2478 |
| 1221b | Seq. ID No. 2483 | Seq. ID No. 2484 |
| 1223 | Seq. ID No. 2485 | Seq. ID No. 2486 |
| 1224c | Seq. ID No. 2491 | Seq. ID No. 2492 |
| 1226 | Seq. ID No. 2495 | Seq. ID No. 2496 |
| 1230 | Seq. ID No. 2505 | Seq. ID No. 2506 |
| 1231 | Seq. ID No. 2507 | Seq. ID No. 2508 |
| 1233c | Seq. ID No. 2513 | Seq. ID No. 2514 |
| 1234c | Seq. ID No. 2519 | Seq. ID No. 2520 |
| 1237 | Seq. ID No. 2525 | Seq. ID No. 2526 |
| 1240 | Seq. ID No. 2529 | Seq. ID No. 2530 |
| 1241c | Seq. ID No. 2535 | Seq. ID No. 2536 |
| 1243 | Seq. ID No. 2537 | Seq. ID No. 2538 |
| 1244 | Seq. ID No. 2539 | Seq. ID No. 2540 |
| 1245e | Seq. ID No. 2549 | Seq. ID No. 2550 |
| 1246b | Seq. ID No. 2553 | Seq. ID No. 2554 |
| 1250e | Seq. ID No. 2563 | Seq. ID No. 2564 |
| 1251b | Seq. ID No. 2567 | Seq. ID No. 2568 |
| 1253d | Seq. ID No. 2575 | Seq. ID No. 2576 |
| 1256 | Seq. ID No. 2577 | Seq. ID No. 2578 |
| 1257b | Seq. ID No. 2581 | Seq. ID No. 2582 |
| 1258d | Seq. ID No. 2589 | Seq. ID No. 2590 |
| 1263 | Seq. ID No. 2593 | Seq. ID No. 2594 |
| 1265 | Seq. ID No. 2595 | Seq. ID No. 2596 |
| 1266b | Seq. ID No. 2599 | Seq. ID No. 2600 |
| 1267b | Seq. ID No. 2603 | Seq. ID No. 2604 |
| 1268b | Seq. ID No. 2607 | Seq. ID No. 2608 |
| 1269 | Seq. ID No. 2609 | Seq. ID No. 2610 |
| 1271 | Seq. ID No. 2613 | Seq. ID No. 2614 |
| 1272b | Seq. ID No. 2617 | Seq. ID No. 2618 |
| 1273 | Seq. ID No. 2619 | Seq. ID No. 2620 |
| 1275b | Seq. ID No. 2623 | Seq. ID No. 2624 |
| 1277 | Seq. ID No. 2625 | Seq. ID No. 2626 |
| 1279 | Seq. ID No. 2631 | Seq. ID No. 2632 |
| 1281 | Seq. ID No. 2633 | Seq. ID No. 2634 |
| 1283c | Seq. ID No. 2639 | Seq. ID No. 2640 |
| 1284 | Seq. ID No. 2641 | Seq. ID No. 2642 |
| 1285b | Seq. ID No. 2645 | Seq. ID No. 2646 |
| 1287b | Seq. ID No. 2649 | Seq. ID No. 2650 |
| 1288 | Seq. ID No. 2651 | Seq. ID No. 2652 |
| 1290 | Seq. ID No. 2655 | Seq. ID No. 2656 |
| 1293b | Seq. ID No. 2659 | Seq. ID No. 2660 |
| 1295 | Seq. ID No. 2663 | Seq. ID No. 2664 |
| 1297b | Seq. ID No. 2667 | Seq. ID No. 2668 |
| 1300 | Seq. ID No. 2669 | Seq. ID No. 2670 |
| 1302 | Seq. ID No. 2671 | Seq. ID No. 2672 |
| 1305c | Seq. ID No. 2677 | Seq. ID No. 2678 |
| 1308e | Seq. ID No. 2687 | Seq. ID No. 2688 |
| 1311 | Seq. ID No. 2689 | Seq. ID No. 2690 |
| 1315 | Seq. ID No. 2693 | Seq. ID No. 2694 |
| 1318 | Seq. ID No. 2699 | Seq. ID No. 2700 |
| 1319b | Seq. ID No. 2703 | Seq. ID No. 2704 |
| 1320c | Seq. ID No. 2709 | Seq. ID No. 2710 |
| 1321 | Seq. ID No. 2711 | Seq. ID No. 2712 |
| 1322b | Seq. ID No. 2715 | Seq. ID No. 2716 |
| 1324 | Seq. ID No. 2719 | Seq. ID No. 2720 |
| 1326c | Seq. ID No. 2725 | Seq. ID No. 2726 |
| 1333 | Seq. ID No. 2727 | Seq. ID No. 2728 |
| 1337 | Seq. ID No. 2737 | Seq. ID No. 2738 |
| 1340b | Seq. ID No. 2743 | Seq. ID No. 2744 |
| 1341c | Seq. ID No. 2747 | Seq. ID No. 2748 |
| 1344b | Seq. ID No. 2751 | Seq. ID No. 2752 |
| 1348 | Seq. ID No. 2753 | Seq. ID No. 2754 |
| 1349c | Seq. ID No. 2759 | Seq. ID No. 2760 |
| 1350b | Seq. ID No. 2763 | Seq. ID No. 2764 |
| 1353 | Seq. ID No. 2765 | Seq. ID No. 2766 |
| 1355 | Seq. ID No. 2767 | Seq. ID No. 2768 |
| 1358b | Seq. ID No. 2771 | Seq. ID No. 2772 |
| 1359 | Seq. ID No. 2773 | Seq. ID No. 2774 |
| 1361 | Seq. ID No. 2775 | Seq. ID No. 2776 |
| 1364c | Seq. ID No. 2781 | Seq. ID No. 2782 |
| 1366b | Seq. ID No. 2787 | Seq. ID No. 2788 |
| 1367 | Seq. ID No. 2789 | Seq. ID No. 2790 |
| 1368 | Seq. ID No. 2791 | Seq. ID No. 2792 |
| 1371 | Seq. ID No. 2793 | Seq. ID No. 2794 |
| 1373b | Seq. ID No. 2797 | Seq. ID No. 2798 |
| 1374 | Seq. ID No. 2799 | Seq. ID No. 2800 |
| 1377 | Seq. ID No. 2803 | Seq. ID No. 2804 |
| 1378 | Seq. ID No. 2805 | Seq. ID No. 2806 |
| 1381a | Seq. ID No. 2811 | Seq. ID No. 2812 |
| 1382 | Seq. ID No. 2815 | Seq. ID No. 2816 |
| 1384c | Seq. ID No. 2823 | Seq. ID No. 2824 |
| 1386b | Seq. ID No. 2827 | Seq. ID No. 2828 |
| 1394 | Seq. ID No. 2843 | Seq. ID No. 2844 |
| 1396 | Seq. ID No. 2847 | Seq. ID No. 2848 |
| 1405 | Seq. ID No. 2863 | Seq. ID No. 2864 |
| 1408 | Seq. ID No. 2867 | Seq. ID No. 2868 |
| 1409 | Seq. ID No. 2869 | Seq. ID No. 2870 |
| 1410 | Seq. ID No. 2871 | Seq. ID No. 2872 |
| 1411c | Seq. ID No. 2877 | Seq. ID No. 2878 |
| 1412b | Seq. ID No. 2881 | Seq. ID No. 2882 |
| 1416 | Seq. ID No. 2891 | Seq. ID No. 2892 |
| 1420b | Seq. ID No. 2899 | Seq. ID No. 2900 |
| 1422 | Seq. ID No. 2901 | Seq. ID No. 2902 |
| 1423 | Seq. ID No. 2903 | Seq. ID No. 2904 |
| 1426b | Seq. ID No. 2907 | Seq. ID No. 2908 |
| 1427b | Seq. ID No. 2911 | Seq. ID No. 2912 |
| 1428 | Seq. ID No. 2913 | Seq. ID No. 2914 |
| 1429b | Seq. ID No. 2917 | Seq. ID No. 2918 |
| 1431 | Seq. ID No. 2919 | Seq. ID No. 2920 |
| 1432 | Seq. ID No. 2921 | Seq. ID No. 2922 |
| 1433 | Seq. ID No. 2923 | Seq. ID No. 2924 |
| 1434b | Seq. ID No. 2927 | Seq. ID No. 2928 |
| 1437c | Seq. ID No. 2933 | Seq. ID No. 2934 |
| 1439e | Seq. ID No. 2945 | Seq. ID No. 2946 |
| 1440d | Seq. ID No. 2953 | Seq. ID No. 2954 |
| 1442b | Seq. ID No. 2957 | Seq. ID No. 2958 |
| 1445c | Seq. ID No. 2963 | Seq. ID No. 2964 |
| 1447b | Seq. ID No. 2971 | Seq. ID No. 2972 |
| 1448 | Seq. ID No. 2973 | Seq. ID No. 2974 |
| 1449b | Seq. ID No. 2977 | Seq. ID No. 2978 |
| 1451 | Seq. ID No. 2979 | Seq. ID No. 2980 |
| 1452b | Seq. ID No. 2983 | Seq. ID No. 2984 |
| 1453 | Seq. ID No. 2985 | Seq. ID No. 2986 |
| 1455b | Seq. ID No. 2991 | Seq. ID No. 2992 |
| 1456 | Seq. ID No. 2993 | Seq. ID No. 2994 |
| 1457c | Seq. ID No. 2999 | Seq. ID No. 3000 |
| 1459 | Seq. ID No. 3001 | Seq. ID No. 3002 |
| 1462b | Seq. ID No. 3005 | Seq. ID No. 3006 |
| 1463c | Seq. ID No. 3011 | Seq. ID No. 3012 |
| 1466d | Seq. ID No. 3019 | Seq. ID No. 3020 |
| 1469c | Seq. ID No. 3027 | Seq. ID No. 3028 |
| 1470c | Seq. ID No. 3033 | Seq. ID No. 3034 |
| 1471 | Seq. ID No. 3035 | Seq. ID No. 3036 |
| 1472c | Seq. ID No. 3039 | Seq. ID No. 3040 |
| 1475 | Seq. ID No. 3043 | Seq. ID No. 3044 |
| 1476 | Seq. ID No. 3045 | Seq. ID No. 3046 |
| 1477b | Seq. ID No. 3049 | Seq. ID No. 3050 |
| 1479c | Seq. ID No. 3055 | Seq. ID No. 3056 |
| 1482b | Seq. ID No. 3059 | Seq. ID No. 3060 |
| 1483 | Seq. ID No. 3061 | Seq. ID No. 3062 |
| 1484c | Seq. ID No. 3067 | Seq. ID No. 3068 |
| 1486 | Seq. ID No. 3069 | Seq. ID No. 3070 |
| 1488e | Seq. ID No. 3081 | Seq. ID No. 3082 |
| 1490 | Seq. ID No. 3089 | Seq. ID No. 3090 |
| 1494 | Seq. ID No. 3091 | Seq. ID No. 3092 |
| 1497 | Seq. ID No. 3093 | Seq. ID No. 3094 |
| 1498b | Seq. ID No. 3097 | Seq. ID No. 3098 |
| 1499 | Seq. ID No. 3103 | Seq. ID No. 3104 |
| 1502b | Seq. ID No. 3107 | Seq. ID No. 3108 |
| 1503b | Seq. ID No. 3111 | Seq. ID No. 3112 |
| 1506c | Seq. ID No. 3117 | Seq. ID No. 3118 |
| 1508 | Seq. ID No. 3125 | Seq. ID No. 3126 |
| 1510d | Seq. ID No. 3133 | Seq. ID No. 3134 |
| 1511 | Seq. ID No. 3135 | Seq. ID No. 3136 |
| 1512 | Seq. ID No. 3137 | Seq. ID No. 3138 |
| 1513b | Seq. ID No. 3141 | Seq. ID No. 3142 |
| 1516 | Seq. ID No. 3143 | Seq. ID No. 3144 |
| 1517c | Seq. ID No. 3149 | Seq. ID No. 3150 |
| 1520 | Seq. ID No. 3155 | Seq. ID No. 3156 |
| 1522 | Seq. ID No. 3157 | Seq. ID No. 3158 |
| 1523 | Seq. ID No. 3159 | Seq. ID No. 3160 |
| 1528 | Seq. ID No. 3161 | Seq. ID No. 3162 |
| 1530b | Seq. ID No. 3165 | Seq. ID No. 3166 |
| 1534b | Seq. ID No. 3171 | Seq. ID No. 3172 |
| 1536b | Seq. ID No. 3177 | Seq. ID No. 3178 |
| 1537 | Seq. ID No. 3179 | Seq. ID No. 3180 |
| 1543 | Seq. ID No. 3183 | Seq. ID No. 3184 |
| 1544 | Seq. ID No. 3185 | Seq. ID No. 3186 |
| 1547b | Seq. ID No. 3189 | Seq. ID No. 3190 |
| 1548 | Seq. ID No. 3191 | Seq. ID No. 3192 |
| 1553b | Seq. ID No. 3197 | Seq. ID No. 3198 |
| 1554c | Seq. ID No. 3203 | Seq. ID No. 3204 |
| 1555 | Seq. ID No. 3205 | Seq. ID No. 3206 |
| 1556c | Seq. ID No. 3211 | Seq. ID No. 3212 |
| 1557c | Seq. ID No. 3217 | Seq. ID No. 3218 |
| 1559b | Seq. ID No. 3221 | Seq. ID No. 3222 |
| 1564b | Seq. ID No. 3227 | Seq. ID No. 3228 |
| 1565 | Seq. ID No. 3229 | Seq. ID No. 3230 |
| 1568b | Seq. ID No. 3233 | Seq. ID No. 3234 |
| 1569 | Seq. ID No. 3235 | Seq. ID No. 3236 |
| 1571 | Seq. ID No. 3239 | Seq. ID No. 3240 |
| 1572e | Seq. ID No. 3249 | Seq. ID No. 3250 |
| 1573 | Seq. ID No. 3251 | Seq. ID No. 3252 |
| 1576 | Seq. ID No. 3253 | Seq. ID No. 3254 |
| 1577b | Seq. ID No. 3257 | Seq. ID No. 3258 |
| 1578 | Seq. ID No. 3259 | Seq. ID No. 3260 |
| 1580b | Seq. ID No. 3263 | Seq. ID No. 3264 |
| 1581 | Seq. ID No. 3265 | Seq. ID No. 3266 |
| 1582c | Seq. ID No. 3271 | Seq. ID No. 3272 |
| 1584 | Seq. ID No. 3273 | Seq. ID No. 3274 |
| 1585b | Seq. ID No. 3277 | Seq. ID No. 3278 |
| 1587c | Seq. ID No. 3283 | Seq. ID No. 3284 |
| 1588 | Seq. ID No. 3285 | Seq. ID No. 3286 |
| 1590 | Seq. ID No. 3287 | Seq. ID No. 3288 |
| 1591 | Seq. ID No. 3289 | Seq. ID No. 3290 |
| 1592b | Seq. ID No. 3293 | Seq. ID No. 3294 |
| 1593 | Seq. ID No. 3295 | Seq. ID No. 3296 |
| 1594b | Seq. ID No. 3299 | Seq. ID No. 3300 |
| 1595b | Seq. ID No. 3303 | Seq. ID No. 3304 |
| 1598b | Seq. ID No. 3307 | Seq. ID No. 3308 |
| 1600c | Seq. ID No. 3313 | Seq. ID No. 3314 |
| 1602 | Seq. ID No. 3315 | Seq. ID No. 3316 |
| 1606b | Seq. ID No. 3319 | Seq. ID No. 3320 |
| 1607b | Seq. ID No. 3323 | Seq. ID No. 3324 |
| 1610b | Seq. ID No. 3329 | Seq. ID No. 3330 |
| 1616b | Seq. ID No. 3335 | Seq. ID No. 3336 |
| 1619 | Seq. ID No. 3337 | Seq. ID No. 3338 |
| 1623c | Seq. ID No. 3347 | Seq. ID No. 3348 |
| 1625c | Seq. ID No. 3355 | Seq. ID No. 3356 |
| 1626b | Seq. ID No. 3359 | Seq. ID No. 3360 |
| 1628b | Seq. ID No. 3363 | Seq. ID No. 3364 |
| 1632b | Seq. ID No. 3367 | Seq. ID No. 3368 |
| 1634c | Seq. ID No. 3377 | Seq. ID No. 3378 |
| 1639b | Seq. ID No. 3379 | Seq. ID No. 3380 |
| 1642b | Seq. ID No. 3383 | Seq. ID No. 3384 |
| 1643b | Seq. ID No. 3387 | Seq. ID No. 3388 |
| 1646f | Seq. ID No. 3399 | Seq. ID No. 3400 |
| 1647 | Seq. ID No. 3401 | Seq. ID No. 3402 |
| 1648b | Seq. ID No. 3403 | Seq. ID No. 3404 |
| 1649c | Seq. ID No. 3409 | Seq. ID No. 3410 |
| 1652b | Seq. ID No. 3413 | Seq. ID No. 3414 |
| 1654 | Seq. ID No. 3415 | Seq. ID No. 3416 |
| 1656b | Seq. ID No. 3417 | Seq. ID No. 3418 |
| 1659c | Seq. ID No. 3425 | Seq. ID No. 3426 |
| 1660b | Seq. ID No. 3427 | Seq. ID No. 3428 |
| 1662b | Seq. ID No. 3431 | Seq. ID No. 3432 |
| 1664 | Seq. ID No. 3435 | Seq. ID No. 3436 |
| 1665a | Seq. ID No. 3437 | Seq. ID No. 3438 |
| 1668 | Seq. ID No. 3439 | Seq. ID No. 3440 |
| 1670c | Seq. ID No. 3447 | Seq. ID No. 3448 |
| 1673f | Seq. ID No. 3459 | Seq. ID No. 3460 |
| 1674 | Seq. ID No. 3461 | Seq. ID No. 3462 |
| 1675c | Seq. ID No. 3467 | Seq. ID No. 3468 |
| 1676 | Seq. ID No. 3469 | Seq. ID No. 3470 |
| 1677 | Seq. ID No. 3471 | Seq. ID No. 3472 |
| 1679 | Seq. ID No. 3473 | Seq. ID No. 3474 |
| 1680 | Seq. ID No. 3475 | Seq. ID No. 3476 |
| 1682b | Seq. ID No. 3479 | Seq. ID No. 3480 |
| 1683b | Seq. ID No. 3483 | Seq. ID No. 3484 |
| 1684 | Seq. ID No. 3485 | Seq. ID No. 3486 |
| 1685 | Seq. ID No. 3487 | Seq. ID No. 3488 |
| 1688c | Seq. ID No. 3495 | Seq. ID No. 3496 |
| 1689c | Seq. ID No. 3501 | Seq. ID No. 3502 |
| 1690b | Seq. ID No. 3505 | Seq. ID No. 3506 |
| 1691 | Seq. ID No. 3507 | Seq. ID No. 3508 |
| 1692 | Seq. ID No. 3509 | Seq. ID No. 3510 |
| 1693 | Seq. ID No. 3511 | Seq. ID No. 3512 |
| 1694d | Seq. ID No. 3519 | Seq. ID No. 3520 |
| 1696 | Seq. ID No. 3521 | Seq. ID No. 3522 |
| 1697b | Seq. ID No. 3525 | Seq. ID No. 3526 |
| 1699b | Seq. ID No. 3529 | Seq. ID No. 3530 |
| 1700b | Seq. ID No. 3533 | Seq. ID No. 3534 |
| 1701b | Seq. ID No. 3537 | Seq. ID No. 3538 |
| 1704b | Seq. ID No. 3543 | Seq. ID No. 3544 |
| 1706 | Seq. ID No. 3549 | Seq. ID No. 3550 |
| 1707b | Seq. ID No. 3553 | Seq. ID No. 3554 |
| 1710b | Seq. ID No. 3561 | Seq. ID No. 3562 |
| 1712b | Seq. ID No. 3565 | Seq. ID No. 3566 |
| 1715b | Seq. ID No. 3569 | Seq. ID No. 3570 |
| 1722 | Seq. ID No. 3575 | Seq. ID No. 3576 |
| 1723b | Seq. ID No. 3579 | Seq. ID No. 3580 |
| 1727 | Seq. ID No. 3583 | Seq. ID No. 3584 |
| 1728 | Seq. ID No. 3585 | Seq. ID No. 3586 |
| 1733 | Seq. ID No. 3589 | Seq. ID No. 3590 |
| 1737b | Seq. ID No. 3599 | Seq. ID No. 3600 |
| 1738c | Seq. ID No. 3605 | Seq. ID No. 3606 |
| 1739 | Seq. ID No. 3607 | Seq. ID No. 3608 |
| 1741 | Seq. ID No. 3609 | Seq. ID No. 3610 |
| 1743 | Seq. ID No. 3611 | Seq. ID No. 3612 |
| 1744b | Seq. ID No. 3615 | Seq. ID No. 3616 |
| 1747 | Seq. ID No. 3619 | Seq. ID No. 3620 |
| 1752b | Seq. ID No. 3629 | Seq. ID No. 3630 |
| 1756b | Seq. ID No. 3635 | Seq. ID No. 3636 |
| 1757b | Seq. ID No. 3639 | Seq. ID No. 3640 |
| 1759 | Seq. ID No. 3641 | Seq. ID No. 3642 |
| 1760 | Seq. ID No. 3643 | Seq. ID No. 3644 |
| 1762 | Seq. ID No. 3645 | Seq. ID No. 3646 |
| 1764c | Seq. ID No. 3651 | Seq. ID No. 3652 |
| 1765 | Seq. ID No. 3653 | Seq. ID No. 3654 |
| 1767b | Seq. ID No. 3659 | Seq. ID No. 3660 |
| 1769b | Seq. ID No. 3663 | Seq. ID No. 3664 |
| 1771 | Seq. ID No. 3665 | Seq. ID No. 3666 |
| 1773 | Seq. ID No. 3667 | Seq. ID No. 3668 |
| 1774 | Seq. ID No. 3669 | Seq. ID No. 3670 |
| 1775c | Seq. ID No. 3675 | Seq. ID No. 3676 |
| 1780 | Seq. ID No. 3681 | Seq. ID No. 3682 |
| 1784b | Seq. ID No. 3689 | Seq. ID No. 3690 |
| 1788c | Seq. ID No. 3695 | Seq. ID No. 3696 |
| 1789b | Seq. ID No. 3699 | Seq. ID No. 3700 |
| 1792c | Seq. ID No. 3709 | Seq. ID No. 3710 |
| 1793c | Seq. ID No. 3715 | Seq. ID No. 3716 |
| 1797b | Seq. ID No. 3721 | Seq. ID No. 3722 |
| 1798c | Seq. ID No. 3727 | Seq. ID No. 3728 |
| 1800 | Seq. ID No. 3729 | Seq. ID No. 3730 |
| 1802b | Seq. ID No. 3733 | Seq. ID No. 3734 |
| 1803 | Seq. ID No. 3735 | Seq. ID No. 3736 |
| 1804b | Seq. ID No. 3739 | Seq. ID No. 3740 |
| 1805 | Seq. ID No. 3741 | Seq. ID No. 3742 |
| 1806c | Seq. ID No. 3747 | Seq. ID No. 3748 |
| 1807b | Seq. ID No. 3751 | Seq. ID No. 3752 |
| 1808d | Seq. ID No. 3759 | Seq. ID No. 3760 |
| 1809f | Seq. ID No. 3771 | Seq. ID No. 3772 |
| 1810b | Seq. ID No. 3775 | Seq. ID No. 3776 |
| 1811b | Seq. ID No. 3779 | Seq. ID No. 3780 |
| 1812b | Seq. ID No. 3783 | Seq. ID No. 3784 |
| 1815c | Seq. ID No. 3791 | Seq. ID No. 3792 |
| 1816c | Seq. ID No. 3797 | Seq. ID No. 3798 |
| 1819b | Seq. ID No. 3803 | Seq. ID No. 3804 |
| 1821 | Seq. ID No. 3805 | Seq. ID No. 3806 |
| 1823b | Seq. ID No. 3811 | Seq. ID No. 3812 |
| 1825 | Seq. ID No. 3813 | Seq. ID No. 3814 |
| 1828 | Seq. ID No. 3819 | Seq. ID No. 3820 |
| 1830b | Seq. ID No. 3823 | Seq. ID No. 3824 |
| 1833 | Seq. ID No. 3825 | Seq. ID No. 3826 |
| 1836b | Seq. ID No. 3833 | Seq. ID No. 3834 |
| 1837 | Seq. ID No. 3835 | Seq. ID No. 3836 |
| 1838d | Seq. ID No. 3843 | Seq. ID No. 3844 |
| 1839b | Seq. ID No. 3847 | Seq. ID No. 3848 |
| 1842d | Seq. ID No. 3855 | Seq. ID No. 3856 |
| 1843 | Seq. ID No. 3857 | Seq. ID No. 3858 |
| 1845c | Seq. ID No. 3863 | Seq. ID No. 3864 |
| 1848b | Seq. ID No. 3867 | Seq. ID No. 3868 |
| 1850c | Seq. ID No. 3873 | Seq. ID No. 3874 |
| 1853b | Seq. ID No. 3877 | Seq. ID No. 3878 |
| 1854b | Seq. ID No. 3881 | Seq. ID No. 3882 |
| 1855 | Seq. ID No. 3883 | Seq. ID No. 3884 |
| 1856 | Seq. ID No. 3885 | Seq. ID No. 3886 |
| 1857d | Seq. ID No. 3893 | Seq. ID No. 3894 |
| 1859 | Seq. ID No. 3897 | Seq. ID No. 3898 |
| 1861b | Seq. ID No. 3901 | Seq. ID No. 3902 |
| 1863b | Seq. ID No. 3907 | Seq. ID No. 3908 |
| 1865b | Seq. ID No. 3913 | Seq. ID No. 3914 |
| 1868 | Seq. ID No. 3915 | Seq. ID No. 3916 |
| 1869 | Seq. ID No. 3917 | Seq. ID No. 3918 |
| 1870 | Seq. ID No. 3919 | Seq. ID No. 3920 |
| 1873c | Seq. ID No. 3925 | Seq. ID No. 3926 |
| 1875c | Seq. ID No. 3929 | Seq. ID No. 3930 |
| 1877b | Seq. ID No. 3933 | Seq. ID No. 3934 |
| 1878 | Seq. ID No. 3935 | Seq. ID No. 3936 |
| 1879 | Seq. ID No. 3937 | Seq. ID No. 3938 |
| 1884 | Seq. ID No. 3941 | Seq. ID No. 3942 |
| 1886 | Seq. ID No. 3943 | Seq. ID No. 3944 |
| 1887 | Seq. ID No. 3945 | Seq. ID No. 3946 |
| 1888b | Seq. ID No. 3949 | Seq. ID No. 3950 |
| 1890 | Seq. ID No. 3951 | Seq. ID No. 3952 |
| 1891b | Seq. ID No. 3955 | Seq. ID No. 3956 |
| 1893 | Seq. ID No. 3957 | Seq. ID No. 3958 |
| 1897c | Seq. ID No. 3971 | Seq. ID No. 3972 |
| 1898b | Seq. ID No. 3975 | Seq. ID No. 3976 |
| 1902b | Seq. ID No. 3983 | Seq. ID No. 3984 |
| 1907 | Seq. ID No. 3985 | Seq. ID No. 3986 |
| 1912 | Seq. ID No. 3987 | Seq. ID No. 3988 |
| 1915b | Seq. ID No. 3991 | Seq. ID No. 3992 |
| 1918d | Seq. ID No. 4003 | Seq. ID No. 4004 |
| 1919c | Seq. ID No. 4009 | Seq. ID No. 4010 |
| 1923b | Seq. ID No. 4013 | Seq. ID No. 4014 |
| 1925 | Seq. ID No. 4015 | Seq. ID No. 4016 |
| 1926 | Seq. ID No. 4017 | Seq. ID No. 4018 |
| 1927b | Seq. ID No. 4021 | Seq. ID No. 4022 |
| 1930 | Seq. ID No. 4023 | Seq. ID No. 4024 |
| 1931c | Seq. ID No. 4029 | Seq. ID No. 4030 |
| 1932 | Seq. ID No. 4031 | Seq. ID No. 4032 |
| 1933b | Seq. ID No. 4035 | Seq. ID No. 4036 |
| 1934 | Seq. ID No. 4037 | Seq. ID No. 4038 |
| 1936 | Seq. ID No. 4039 | Seq. ID No. 4040 |
| 1937e | Seq. ID No. 4049 | Seq. ID No. 4050 |
| 1939c | Seq. ID No. 4055 | Seq. ID No. 4056 |
| 1940 | Seq. ID No. 4057 | Seq. ID No. 4058 |
| 1941b | Seq. ID No. 4061 | Seq. ID No. 4062 |
| 1942 | Seq. ID No. 4063 | Seq. ID No. 4064 |
| 1943b | Seq. ID No. 4067 | Seq. ID No. 4068 |
| 1944 | Seq. ID No. 4069 | Seq. ID No. 4070 |
| 1946c | Seq. ID No. 4075 | Seq. ID No. 4076 |
| 1949 | Seq. ID No. 4077 | Seq. ID No. 4078 |
| 1950 | Seq. ID No. 4079 | Seq. ID No. 4080 |
| 1951d | Seq. ID No. 4087 | Seq. ID No. 4088 |
| 1953 | Seq. ID No. 4089 | Seq. ID No. 4090 |
| 1954c | Seq. ID No. 4095 | Seq. ID No. 4096 |
| 1956 | Seq. ID No. 4097 | Seq. ID No. 4098 |
| 1957 | Seq. ID No. 4099 | Seq. ID No. 4100 |
| 1958c | Seq. ID No. 4105 | Seq. ID No. 4106 |
| 1960c | Seq. ID No. 4111 | Seq. ID No. 4112 |
| 1962b | Seq. ID No. 4117 | Seq. ID No. 4118 |
| 1965b | Seq. ID No. 4121 | Seq. ID No. 4122 |
| 1966 | Seq. ID No. 4123 | Seq. ID No. 4124 |
| 1968 | Seq. ID No. 4127 | Seq. ID No. 4128 |
| 1969 | Seq. ID No. 4129 | Seq. ID No. 4130 |
| 1970b | Seq. ID No. 4133 | Seq. ID No. 4134 |
| 1973 | Seq. ID No. 4137 | Seq. ID No. 4138 |
| 1977c | Seq. ID No. 4145 | Seq. ID No. 4146 |
| 1979b | Seq. ID No. 4149 | Seq. ID No. 4150 |
| 1982 | Seq. ID No. 4157 | Seq. ID No. 4158 |
| 1985b | Seq. ID No. 4161 | Seq. ID No. 4162 |
| 1986 | Seq. ID No. 4163 | Seq. ID No. 4164 |
| 1987b | Seq. ID No. 4167 | Seq. ID No. 4168 |
| 1989c | Seq. ID No. 4173 | Seq. ID No. 4174 |
| 1990c | Seq. ID No. 4179 | Seq. ID No. 4180 |
| 1992b | Seq. ID No. 4185 | Seq. ID No. 4186 |
| 1993c | Seq. ID No. 4191 | Seq. ID No. 4192 |
| 1996 | Seq. ID No. 4193 | Seq. ID No. 4194 |
| 2000 | Seq. ID No. 4195 | Seq. ID No. 4196 |
| 2001 | Seq. ID No. 4197 | Seq. ID No. 4198 |
| 2003 | Seq. ID No. 4199 | Seq. ID No. 4200 |
| 2006 | Seq. ID No. 4201 | Seq. ID No. 4202 |
| 2008 | Seq. ID No. 4203 | Seq. ID No. 4204 |
| 2010 | Seq. ID No. 4205 | Seq. ID No. 4206 |
| 2011c | Seq. ID No. 4211 | Seq. ID No. 4212 |
| 2013b | Seq. ID No. 4215 | Seq. ID No. 4216 |
| 2014c | Seq. ID No. 4217 | Seq. ID No. 4218 |
| 2015 | Seq. ID No. 4219 | Seq. ID No. 4220 |
| 2016h | Seq. ID No. 4235 | Seq. ID No. 4236 |
| 2020 | Seq. ID No. 4237 | Seq. ID No. 4238 |
| 2021 | Seq. ID No. 4239 | Seq. ID No. 4240 |
| 2023b | Seq. ID No. 4243 | Seq. ID No. 4244 |
| 2024d | Seq. ID No. 4251 | Seq. ID No. 4252 |
| 2027 | Seq. ID No. 4255 | Seq. ID No. 4256 |
| 2030d | Seq. ID No. 4263 | Seq. ID No. 4264 |
| 2033 | Seq. ID No. 4267 | Seq. ID No. 4268 |
| 2036 | Seq. ID No. 4271 | Seq. ID No. 4272 |
| 2037 | Seq. ID No. 4273 | Seq. ID No. 4274 |
| 2042 | Seq. ID No. 4279 | Seq. ID No. 4280 |
| 2043b | Seq. ID No. 4281 | Seq. ID No. 4282 |
| 2044 | Seq. ID No. 4283 | Seq. ID No. 4284 |
| 2046b | Seq. ID No. 4291 | Seq. ID No. 4292 |
| 2047b | Seq. ID No. 4295 | Seq. ID No. 4296 |
| 2050 | Seq. ID No. 4299 | Seq. ID No. 4300 |
| 2053 | Seq. ID No. 4301 | Seq. ID No. 4302 |
| 2054 | Seq. ID No. 4303 | Seq. ID No. 4304 |
| 2056 | Seq. ID No. 4307 | Seq. ID No. 4308 |
| 2057 | Seq. ID No. 4309 | Seq. ID No. 4310 |
| 2059b | Seq. ID No. 4313 | Seq. ID No. 4314 |
| 2060b | Seq. ID No. 4317 | Seq. ID No. 4318 |
| 2062c | Seq. ID No. 4323 | Seq. ID No. 4324 |
| 2064 | Seq. ID No. 4325 | Seq. ID No. 4326 |
| 2065b | Seq. ID No. 4329 | Seq. ID No. 4330 |
| 2066 | Seq. ID No. 4331 | Seq. ID No. 4332 |
| 2068 | Seq. ID No. 4333 | Seq. ID No. 4334 |
| 2069c | Seq. ID No. 4339 | Seq. ID No. 4340 |
| 2074b | Seq. ID No. 4343 | Seq. ID No. 4344 |
| 2075b | Seq. ID No. 4347 | Seq. ID No. 4348 |
| 2076c | Seq. ID No. 4353 | Seq. ID No. 4354 |
| 2078 | Seq. ID No. 4355 | Seq. ID No. 4356 |
| 2079a | Seq. ID No. 4357 | Seq. ID No. 4358 |
| 2081b | Seq. ID No. 4365 | Seq. ID No. 4366 |
| 2086b | Seq. ID No. 4369 | Seq. ID No. 4370 |
| 2087b | Seq. ID No. 4373 | Seq. ID No. 4374 |
| 2088 | Seq. ID No. 4375 | Seq. ID No. 4376 |
| 2091 | Seq. ID No. 4377 | Seq. ID No. 4378 |
| 2092 | Seq. ID No. 4379 | Seq. ID No. 4380 |
| 2093a | Seq. ID No. 4381 | Seq. ID No. 4382 |
| 2094 | Seq. ID No. 4383 | Seq. ID No. 4384 |
| 2096d | Seq. ID No. 4391 | Seq. ID No. 4392 |
| 2097 | Seq. ID No. 4393 | Seq. ID No. 4394 |
| 2100 | Seq. ID No. 4397 | Seq. ID No. 4398 |
| 2104 | Seq. ID No. 4401 | Seq. ID No. 4402 |
| 2109b | Seq. ID No. 4411 | Seq. ID No. 4412 |
| 2110c | Seq. ID No. 4417 | Seq. ID No. 4418 |
| 2112c | Seq. ID No. 4423 | Seq. ID No. 4424 |
| 2115c | Seq. ID No. 4431 | Seq. ID No. 4432 |
| 2116 | Seq. ID No. 4433 | Seq. ID No. 4434 |
| 2117 | Seq. ID No. 4435 | Seq. ID No. 4436 |
| 2123 | Seq. ID No. 4443 | Seq. ID No. 4444 |
| 2124b | Seq. ID No. 4447 | Seq. ID No. 4448 |
| 2125b | Seq. ID No. 4451 | Seq. ID No. 4452 |
| 2128b | Seq. ID No. 4459 | Seq. ID No. 4460 |
| 2133 | Seq. ID No. 4461 | Seq. ID No. 4462 |
| 2138c | Seq. ID No. 4467 | Seq. ID No. 4468 |
| 2140b | Seq. ID No. 4471 | Seq. ID No. 4472 |
| 2142b | Seq. ID No. 4475 | Seq. ID No. 4476 |
| 2144b | Seq. ID No. 4479 | Seq. ID No. 4480 |
| 2146 | Seq. ID No. 4481 | Seq. ID No. 4482 |
| 2147c | Seq. ID No. 4487 | Seq. ID No. 4488 |
| 2149b | Seq. ID No. 4491 | Seq. ID No. 4492 |
| 2153 | Seq. ID No. 4501 | Seq. ID No. 4502 |
| 2155b | Seq. ID No. 4505 | Seq. ID No. 4506 |
| 2156c | Seq. ID No. 4511 | Seq. ID No. 4512 |
| 2160d | Seq. ID No. 4523 | Seq. ID No. 4524 |
| 2164 | Seq. ID No. 4531 | Seq. ID No. 4532 |
| 2165 | Seq. ID No. 4533 | Seq. ID No. 4534 |
| 2166b | Seq. ID No. 4537 | Seq. ID No. 4538 |
| 2169 | Seq. ID No. 4539 | Seq. ID No. 4540 |
| 2171 | Seq. ID No. 4541 | Seq. ID No. 4542 |
| 2172 | Seq. ID No. 4543 | Seq. ID No. 4544 |
| 2173b | Seq. ID No. 4547 | Seq. ID No. 4548 |
| 2174 | Seq. ID No. 4549 | Seq. ID No. 4550 |
| 2176d | Seq. ID No. 4559 | Seq. ID No. 4560 |
| 2179b | Seq. ID No. 4565 | Seq. ID No. 4566 |
| 2180 | Seq. ID No. 4567 | Seq. ID No. 4568 |
| 2181b | Seq. ID No. 4571 | Seq. ID No. 4572 |
| 2183 | Seq. ID No. 4575 | Seq. ID No. 4576 |
| 2185 | Seq. ID No. 4577 | Seq. ID No. 4578 |
| 2186b | Seq. ID No. 4581 | Seq. ID No. 4582 |
| 2189b | Seq. ID No. 4585 | Seq. ID No. 4586 |
| 2193 | Seq. ID No. 4591 | Seq. ID No. 4592 |
| 2194c | Seq. ID No. 4595 | Seq. ID No. 4596 |
| 2196 | Seq. ID No. 4597 | Seq. ID No. 4598 |
| 2197c | Seq. ID No. 4603 | Seq. ID No. 4604 |
| 2201b | Seq. ID No. 4607 | Seq. ID No. 4608 |
| 2203 | Seq. ID No. 4609 | Seq. ID No. 4610 |
| 2204 | Seq. ID No. 4611 | Seq. ID No. 4612 |
| 2206b | Seq. ID No. 4615 | Seq. ID No. 4616 |
| 2209 | Seq. ID No. 4623 | Seq. ID No. 4624 |
| 2212b | Seq. ID No. 4627 | Seq. ID No. 4628 |
| 2215b | Seq. ID No. 4631 | Seq. ID No. 4632 |
| 2216b | Seq. ID No. 4635 | Seq. ID No. 4636 |
| 2218 | Seq. ID No. 4637 | Seq. ID No. 4638 |
| 2219c | Seq. ID No. 4643 | Seq. ID No. 4644 |
| 2220c | Seq. ID No. 4649 | Seq. ID No. 4650 |
| 2221 | Seq. ID No. 4651 | Seq. ID No. 4652 |
| 2222 | Seq. ID No. 4653 | Seq. ID No. 4654 |
| 2223b | Seq. ID No. 4657 | Seq. ID No. 4658 |
| 2226 | Seq. ID No. 4661 | Seq. ID No. 4662 |
| 2227 | Seq. ID No. 4663 | Seq. ID No. 4664 |
| 2228c | Seq. ID No. 4669 | Seq. ID No. 4670 |
| 2231 | Seq. ID No. 4671 | Seq. ID No. 4672 |
| 2232 | Seq. ID No. 4673 | Seq. ID No. 4674 |
| 2234c | Seq. ID No. 4679 | Seq. ID No. 4680 |
| 2238 | Seq. ID No. 4685 | Seq. ID No. 4686 |
| 2240c | Seq. ID No. 4693 | Seq. ID No. 4694 |
| 2244 | Seq. ID No. 4697 | Seq. ID No. 4698 |
| 2248 | Seq. ID No. 4703 | Seq. ID No. 4704 |
| 2249 | Seq. ID No. 4705 | Seq. ID No. 4706 |
| 2253d | Seq. ID No. 4713 | Seq. ID No. 4714 |
| 2255b | Seq. ID No. 4717 | Seq. ID No. 4718 |
| 2256 | Seq. ID No. 4719 | Seq. ID No. 4720 |
| 2259b | Seq. ID No. 4725 | Seq. ID No. 4726 |
| 2260c | Seq. ID No. 4729 | Seq. ID No. 4730 |
| 2262 | Seq. ID No. 4731 | Seq. ID No. 4732 |
| 2263 | Seq. ID No. 4733 | Seq. ID No. 4734 |
| 2267 | Seq. ID No. 4739 | Seq. ID No. 4740 |
| 2269 | Seq. ID No. 4743 | Seq. ID No. 4744 |
| 2270 | Seq. ID No. 4745 | Seq. ID No. 4746 |
| 2272 | Seq. ID No. 4747 | Seq. ID No. 4748 |
| 2273 | Seq. ID No. 4749 | Seq. ID No. 4750 |
| 2274 | Seq. ID No. 4751 | Seq. ID No. 4752 |
| 2275c | Seq. ID No. 4757 | Seq. ID No. 4758 |
| 2276d | Seq. ID No. 4765 | Seq. ID No. 4766 |
| 2277b | Seq. ID No. 4769 | Seq. ID No. 4770 |
| 2278d | Seq. ID No. 4777 | Seq. ID No. 4778 |
| 2280 | Seq. ID No. 4779 | Seq. ID No. 4780 |
| 2284 | Seq. ID No. 4781 | Seq. ID No. 4782 |
| 2286b | Seq. ID No. 4787 | Seq. ID No. 4788 |
| 2288 | Seq. ID No. 4789 | Seq. ID No. 4790 |
| 2290 | Seq. ID No. 4791 | Seq. ID No. 4792 |
| 2291c | Seq. ID No. 4797 | Seq. ID No. 4798 |
| 2292c | Seq. ID No. 4803 | Seq. ID No. 4804 |
| 2297 | Seq. ID No. 4807 | Seq. ID No. 4808 |
| 2298 | Seq. ID No. 4809 | Seq. ID No. 4810 |
| 2299 | Seq. ID No. 4811 | Seq. ID No. 4812 |
| 2300 | Seq. ID No. 4813 | Seq. ID No. 4814 |
| 2301 | Seq. ID No. 4815 | Seq. ID No. 4816 |
| 2304 | Seq. ID No. 4821 | Seq. ID No. 4822 |
| 2306c | Seq. ID No. 4823 | Seq. ID No. 4824 |
| 2307 | Seq. ID No. 4825 | Seq. ID No. 4826 |
| 2308 | Seq. ID No. 4827 | Seq. ID No. 4828 |
| 2309c | Seq. ID No. 4833 | Seq. ID No. 4834 |
| 2310 | Seq. ID No. 4835 | Seq. ID No. 4836 |
| 2311 | Seq. ID No. 4837 | Seq. ID No. 4838 |
| 2312c | Seq. ID No. 4843 | Seq. ID No. 4844 |
| 2314 | Seq. ID No. 4845 | Seq. ID No. 4846 |
| 2315 | Seq. ID No. 4847 | Seq. ID No. 4848 |
| 2316 | Seq. ID No. 4849 | Seq. ID No. 4850 |
| 2321c | Seq. ID No. 4859 | Seq. ID No. 4860 |
| 2322 | Seq. ID No. 4861 | Seq. ID No. 4862 |
| 2327 | Seq. ID No. 4865 | Seq. ID No. 4866 |
| 2328 | Seq. ID No. 4867 | Seq. ID No. 4868 |
| 2329d | Seq. ID No. 4875 | Seq. ID No. 4876 |
| 2330 | Seq. ID No. 4877 | Seq. ID No. 4878 |
| 2331 | Seq. ID No. 4879 | Seq. ID No. 4880 |
| 2333 | Seq. ID No. 4881 | Seq. ID No. 4882 |
| 2335 | Seq. ID No. 4883 | Seq. ID No. 4884 |
| 2336 | Seq. ID No. 4885 | Seq. ID No. 4886 |
| 2337b | Seq. ID No. 4887 | Seq. ID No. 4888 |
| 2338c | Seq. ID No. 4893 | Seq. ID No. 4894 |
| 2342c | Seq. ID No. 4899 | Seq. ID No. 4900 |
| 2344 | Seq. ID No. 4901 | Seq. ID No. 4902 |
| 2345 | Seq. ID No. 4903 | Seq. ID No. 4904 |
| 2347 | Seq. ID No. 4905 | Seq. ID No. 4906 |
| 2348 | Seq. ID No. 4907 | Seq. ID No. 4908 |
| 2349b | Seq. ID No. 4911 | Seq. ID No. 4912 |
| 2353 | Seq. ID No. 4915 | Seq. ID No. 4916 |
| 2354 | Seq. ID No. 4917 | Seq. ID No. 4918 |
| 2357 | Seq. ID No. 4919 | Seq. ID No. 4920 |
| 2358 | Seq. ID No. 4921 | Seq. ID No. 4922 |
| 2361b | Seq. ID No. 4927 | Seq. ID No. 4928 |
| 2363b | Seq. ID No. 4931 | Seq. ID No. 4932 |
| 2365b | Seq. ID No. 4935 | Seq. ID No. 4936 |
| 2366b | Seq. ID No. 4939 | Seq. ID No. 4940 |
| 2367 | Seq. ID No. 4941 | Seq. ID No. 4942 |
| 2368 | Seq. ID No. 4943 | Seq. ID No. 4944 |
| 2369 | Seq. ID No. 4945 | Seq. ID No. 4946 |
| 2370 | Seq. ID No. 4947 | Seq. ID No. 4948 |
| 2373 | Seq. ID No. 4953 | Seq. ID No. 4954 |
| 2374b | Seq. ID No. 4957 | Seq. ID No. 4958 |
| 2376 | Seq. ID No. 4959 | Seq. ID No. 4960 |
| 2377b | Seq. ID No. 4963 | Seq. ID No. 4964 |
| 2378 | Seq. ID No. 4965 | Seq. ID No. 4966 |
| 2379b | Seq. ID No. 4969 | Seq. ID No. 4970 |
| 2380 | Seq. ID No. 4971 | Seq. ID No. 4972 |
| 2381 | Seq. ID No. 4973 | Seq. ID No. 4974 |
| 2384b | Seq. ID No. 4979 | Seq. ID No. 4980 |
| 2387 | Seq. ID No. 4985 | Seq. ID No. 4986 |
| 2391 | Seq. ID No. 4989 | Seq. ID No. 4990 |
| 2392 | Seq. ID No. 4991 | Seq. ID No. 4992 |
| 2395 | Seq. ID No. 4995 | Seq. ID No. 4996 |
| 2396 | Seq. ID No. 4997 | Seq. ID No. 4998 |
| 2398 | Seq. ID No. 5001 | Seq. ID No. 5002 |
| 2399c | Seq. ID No. 5007 | Seq. ID No. 5008 |
| 2401 | Seq. ID No. 5009 | Seq. ID No. 5010 |
| 2403b | Seq. ID No. 5017 | Seq. ID No. 5018 |
| 2404b | Seq. ID No. 5021 | Seq. ID No. 5022 |
| 2409b | Seq. ID No. 5027 | Seq. ID No. 5028 |
| 2410 | Seq. ID No. 5029 | Seq. ID No. 5030 |
| 2415b | Seq. ID No. 5039 | Seq. ID No. 5040 |
| 2417b | Seq. ID No. 5047 | Seq. ID No. 5048 |
| 2418b | Seq. ID No. 5051 | Seq. ID No. 5052 |
| 2420 | Seq. ID No. 5053 | Seq. ID No. 5054 |
| 2421 | Seq. ID No. 5055 | Seq. ID No. 5056 |
| 2422c | Seq. ID No. 5059 | Seq. ID No. 5060 |
| 2423a | Seq. ID No. 5061 | Seq. ID No. 5062 |
| 2424c | Seq. ID No. 5069 | Seq. ID No. 5070 |
| 2428c | Seq. ID No. 5077 | Seq. ID No. 5078 |
| 2429 | Seq. ID No. 5079 | Seq. ID No. 5080 |
| 2432 | Seq. ID No. 5081 | Seq. ID No. 5082 |
| 2433 | Seq. ID No. 5083 | Seq. ID No. 5084 |
| 2440 | Seq. ID No. 5087 | Seq. ID No. 5088 |
| 2442b | Seq. ID No. 5091 | Seq. ID No. 5092 |
| 2443 | Seq. ID No. 5093 | Seq. ID No. 5094 |
| 2444b | Seq. ID No. 5097 | Seq. ID No. 5098 |
| 2445 | Seq. ID No. 5103 | Seq. ID No. 5104 |
| 2448b | Seq. ID No. 5105 | Seq. ID No. 5106 |
| 2449b | Seq. ID No. 5109 | Seq. ID No. 5110 |
| 2451 | Seq. ID No. 5111 | Seq. ID No. 5112 |
| 2452 | Seq. ID No. 5113 | Seq. ID No. 5114 |
| 2453 | Seq. ID No. 5115 | Seq. ID No. 5116 |
| 2455 | Seq. ID No. 5117 | Seq. ID No. 5118 |
| 2456e | Seq. ID No. 5127 | Seq. ID No. 5128 |
| 2458g | Seq. ID No. 5141 | Seq. ID No. 5142 |
| 2461b | Seq. ID No. 5145 | Seq. ID No. 5146 |
| 2462b | Seq. ID No. 5149 | Seq. ID No. 5150 |
| 2464 | Seq. ID No. 5153 | Seq. ID No. 5154 |
| 2465b | Seq. ID No. 5157 | Seq. ID No. 5158 |
| 2469b | Seq. ID No. 5165 | Seq. ID No. 5166 |
| 2471 | Seq. ID No. 5169 | Seq. ID No. 5170 |
| 2473 | Seq. ID No. 5171 | Seq. ID No. 5172 |
| 2474c | Seq. ID No. 5177 | Seq. ID No. 5178 |
| 2475c | Seq. ID No. 5183 | Seq. ID No. 5184 |
| 2477 | Seq. ID No. 5185 | Seq. ID No. 5186 |
| 2480 | Seq. ID No. 5187 | Seq. ID No. 5188 |
| 2482 | Seq. ID No. 5193 | Seq. ID No. 5194 |
| 2483 | Seq. ID No. 5195 | Seq. ID No. 5196 |
| 2485 | Seq. ID No. 5201 | Seq. ID No. 5202 |
| 2489 | Seq. ID No. 5203 | Seq. ID No. 5204 |
| 2490 | Seq. ID No. 5205 | Seq. ID No. 5206 |
| 2492b | Seq. ID No. 5209 | Seq. ID No. 5210 |
| 2494 | Seq. ID No. 5211 | Seq. ID No. 5212 |
| 2495b | Seq. ID No. 5215 | Seq. ID No. 5216 |
| 2497b | Seq. ID No. 5219 | Seq. ID No. 5220 |
| 2500c | Seq. ID No. 5227 | Seq. ID No. 5228 |
| 2501b | Seq. ID No. 5231 | Seq. ID No. 5232 |
| 2504b | Seq. ID No. 5235 | Seq. ID No. 5236 |
| 2505 | Seq. ID No. 5237 | Seq. ID No. 5238 |
| 2506 | Seq. ID No. 5239 | Seq. ID No. 5240 |
| 2507c | Seq. ID No. 5243 | Seq. ID No. 5244 |
| 2510 | Seq. ID No. 5245 | Seq. ID No. 5246 |
| 2512c | Seq. ID No. 5247 | Seq. ID No. 5248 |
| 2513c | Seq. ID No. 5253 | Seq. ID No. 5254 |
| 2514 | Seq. ID No. 5255 | Seq. ID No. 5256 |
| 2515 | Seq. ID No. 5257 | Seq. ID No. 5258 |
| 2516b | Seq. ID No. 5261 | Seq. ID No. 5262 |
| 2517a | Seq. ID No. 5263 | Seq. ID No. 5264 |
| 2519b | Seq. ID No. 5271 | Seq. ID No. 5272 |
| 2520b | Seq. ID No. 5275 | Seq. ID No. 5276 |
| 2523 | Seq. ID No. 5283 | Seq. ID No. 5284 |
| 2527b | Seq. ID No. 5291 | Seq. ID No. 5292 |
| 2528d | Seq. ID No. 5299 | Seq. ID No. 5300 |
| 2534 | Seq. ID No. 5305 | Seq. ID No. 5306 |
| 2535b | Seq. ID No. 5309 | Seq. ID No. 5310 |
| 2538 | Seq. ID No. 5311 | Seq. ID No. 5312 |
| 2541 | Seq. ID No. 5313 | Seq. ID No. 5314 |
| 2542c | Seq. ID No. 5319 | Seq. ID No. 5320 |
| 2544 | Seq. ID No. 5323 | Seq. ID No. 5324 |
| 2545 | Seq. ID No. 5325 | Seq. ID No. 5326 |
| 2548b | Seq. ID No. 5335 | Seq. ID No. 5336 |
| 2549 | Seq. ID No. 5337 | Seq. ID No. 5338 |
| 2550c | Seq. ID No. 5341 | Seq. ID No. 5342 |
| 2552 | Seq. ID No. 5345 | Seq. ID No. 5346 |
| 2554b | Seq. ID No. 5351 | Seq. ID No. 5352 |
| 2555b | Seq. ID No. 5355 | Seq. ID No. 5356 |
| 2558b | Seq. ID No. 5363 | Seq. ID No. 5364 |
| 2559 | Seq. ID No. 5365 | Seq. ID No. 5366 |
| 2561 | Seq. ID No. 5367 | Seq. ID No. 5368 |
| 2562c | Seq. ID No. 5371 | Seq. ID No. 5372 |
| 2563 | Seq. ID No. 5373 | Seq. ID No. 5374 |
| 2564d | Seq. ID No. 5381 | Seq. ID No. 5382 |
| 2566 | Seq. ID No. 5383 | Seq. ID No. 5384 |
| 2567b | Seq. ID No. 5387 | Seq. ID No. 5388 |
| 2568 | Seq. ID No. 5389 | Seq. ID No. 5390 |
| 2572 | Seq. ID No. 5391 | Seq. ID No. 5392 |
| 2573 | Seq. ID No. 5393 | Seq. ID No. 5394 |
| 2575 | Seq. ID No. 5395 | Seq. ID No. 5396 |
| 2576 | Seq. ID No. 5397 | Seq. ID No. 5398 |
| 2578 | Seq. ID No. 5399 | Seq. ID No. 5400 |
| 2579b | Seq. ID No. 5401 | Seq. ID No. 5402 |
| 2581 | Seq. ID No. 5403 | Seq. ID No. 5404 |
| 2582c | Seq. ID No. 5409 | Seq. ID No. 5410 |
| 2585 | Seq. ID No. 5411 | Seq. ID No. 5412 |
| 2587b | Seq. ID No. 5415 | Seq. ID No. 5416 |
| 2588 | Seq. ID No. 5417 | Seq. ID No. 5418 |
| 2589 | Seq. ID No. 5419 | Seq. ID No. 5420 |
| 2591 | Seq. ID No. 5421 | Seq. ID No. 5422 |
| 2592 | Seq. ID No. 5423 | Seq. ID No. 5424 |
| 2593b | Seq. ID No. 5427 | Seq. ID No. 5428 |
| 2594b | Seq. ID No. 5431 | Seq. ID No. 5432 |
| 2596 | Seq. ID No. 5433 | Seq. ID No. 5434 |
| 2597c | Seq. ID No. 5439 | Seq. ID No. 5440 |
| 2598 | Seq. ID No. 5441 | Seq. ID No. 5442 |
| 2602 | Seq. ID No. 5443 | Seq. ID No. 5444 |
| 2605b | Seq. ID No. 5447 | Seq. ID No. 5448 |
| 2606 | Seq. ID No. 5449 | Seq. ID No. 5450 |
| 2608 | Seq. ID No. 5451 | Seq. ID No. 5452 |
| 2610 | Seq. ID No. 5453 | Seq. ID No. 5454 |
| 2611 | Seq. ID No. 5455 | Seq. ID No. 5456 |
| 2613 | Seq. ID No. 5457 | Seq. ID No. 5458 |
| 2616h | Seq. ID No. 5473 | Seq. ID No. 5474 |
| 2617b | Seq. ID No. 5477 | Seq. ID No. 5478 |
| 2619 | Seq. ID No. 5483 | Seq. ID No. 5484 |
| 2621a | Seq. ID No. 5487 | Seq. ID No. 5488 |
| 2623b | Seq. ID No. 5491 | Seq. ID No. 5492 |
| 2624b | Seq. ID No. 5495 | Seq. ID No. 5496 |
| 2625b | Seq. ID No. 5499 | Seq. ID No. 5500 |
| 2626b | Seq. ID No. 5503 | Seq. ID No. 5504 |
| 2627b | Seq. ID No. 5507 | Seq. ID No. 5508 |
| 2628 | Seq. ID No. 5509 | Seq. ID No. 5510 |
| 2629b | Seq. ID No. 5513 | Seq. ID No. 5514 |
| 2633e | Seq. ID No. 5527 | Seq. ID No. 5528 |
| 2637b | Seq. ID No. 5531 | Seq. ID No. 5532 |
| 2643b | Seq. ID No. 5537 | Seq. ID No. 5538 |
| 2645 | Seq. ID No. 5539 | Seq. ID No. 5540 |
| 2647 | Seq. ID No. 5541 | Seq. ID No. 5542 |
| 2649 | Seq. ID No. 5547 | Seq. ID No. 5548 |
| 2652b | Seq. ID No. 5553 | Seq. ID No. 5554 |
| 2655b | Seq. ID No. 5557 | Seq. ID No. 5558 |
| 2656 | Seq. ID No. 5559 | Seq. ID No. 5560 |
| 2658b | Seq. ID No. 5563 | Seq. ID No. 5564 |
| 2659b | Seq. ID No. 5567 | Seq. ID No. 5568 |
| 2662 | Seq. ID No. 5571 | Seq. ID No. 5572 |
| 2664b | Seq. ID No. 5575 | Seq. ID No. 5576 |
| 2666b | Seq. ID No. 5581 | Seq. ID No. 5582 |
| 2667b | Seq. ID No. 5585 | Seq. ID No. 5586 |
| 2669c | Seq. ID No. 5591 | Seq. ID No. 5592 |
| 2674b | Seq. ID No. 5599 | Seq. ID No. 5600 |
| 2676 | Seq. ID No. 5601 | Seq. ID No. 5602 |
| 2677b | Seq. ID No. 5605 | Seq. ID No. 5606 |
| 2679d | Seq. ID No. 5615 | Seq. ID No. 5616 |
| 2680c | Seq. ID No. 5621 | Seq. ID No. 5622 |
| 2684b | Seq. ID No. 5629 | Seq. ID No. 5630 |
| 2686 | Seq. ID No. 5631 | Seq. ID No. 5632 |
| 2688c | Seq. ID No. 5637 | Seq. ID No. 5638 |
| 2690c | Seq. ID No. 5647 | Seq. ID No. 5648 |
| 2693 | Seq. ID No. 5651 | Seq. ID No. 5652 |
| 2694b | Seq. ID No. 5655 | Seq. ID No. 5656 |
| 2695 | Seq. ID No. 5657 | Seq. ID No. 5658 |
| 2696b | Seq. ID No. 5661 | Seq. ID No. 5662 |
| 2709 | Seq. ID No. 5665 | Seq. ID No. 5666 |
| 2712 | Seq. ID No. 5669 | Seq. ID No. 5670 |
| 2713 | Seq. ID No. 5671 | Seq. ID No. 5672 |
| 2714 | Seq. ID No. 5673 | Seq. ID No. 5674 |
| 2716c | Seq. ID No. 5679 | Seq. ID No. 5680 |
| 2718c | Seq. ID No. 5685 | Seq. ID No. 5686 |
| 2721b | Seq. ID No. 5693 | Seq. ID No. 5694 |
| 2722b | Seq. ID No. 5697 | Seq. ID No. 5698 |
| 2723 | Seq. ID No. 5699 | Seq. ID No. 5700 |
| 2724 | Seq. ID No. 5701 | Seq. ID No. 5702 |
| 2726 | Seq. ID No. 5707 | Seq. ID No. 5708 |
| 2731b | Seq. ID No. 5715 | Seq. ID No. 5716 |
| 2732 | Seq. ID No. 5717 | Seq. ID No. 5718 |
| 2734c | Seq. ID No. 5723 | Seq. ID No. 5724 |
| 2735b | Seq. ID No. 5727 | Seq. ID No. 5728 |
| 2738d | Seq. ID No. 5735 | Seq. ID No. 5736 |
| 2742b | Seq. ID No. 5739 | Seq. ID No. 5740 |
| 2745b | Seq. ID No. 5749 | Seq. ID No. 5750 |
| 2746 | Seq. ID No. 5751 | Seq. ID No. 5752 |
| 2747b | Seq. ID No. 5755 | Seq. ID No. 5756 |
| 2748c | Seq. ID No. 5761 | Seq. ID No. 5762 |
| 2749e | Seq. ID No. 5771 | Seq. ID No. 5772 |
| 2750b | Seq. ID No. 5775 | Seq. ID No. 5776 |
| 2752 | Seq. ID No. 5777 | Seq. ID No. 5778 |
| 2753 | Seq. ID No. 5779 | Seq. ID No. 5780 |
| 2757b | Seq. ID No. 5783 | Seq. ID No. 5784 |
| 2758c | Seq. ID No. 5789 | Seq. ID No. 5790 |
| 2759b | Seq. ID No. 5795 | Seq. ID No. 5796 |
| 2760c | Seq. ID No. 5801 | Seq. ID No. 5802 |
| 2762 | Seq. ID No. 5803 | Seq. ID No. 5804 |
| 2763c | Seq. ID No. 5809 | Seq. ID No. 5810 |
| 2765c | Seq. ID No. 5815 | Seq. ID No. 5816 |
| 2766b | Seq. ID No. 5819 | Seq. ID No. 5820 |
| 2768c | Seq. ID No. 5825 | Seq. ID No. 5826 |
| 2771b | Seq. ID No. 5833 | Seq. ID No. 5834 |
| 2773 | Seq. ID No. 5835 | Seq. ID No. 5836 |
| 2774c | Seq. ID No. 5841 | Seq. ID No. 5842 |
| 2776b | Seq. ID No. 5845 | Seq. ID No. 5846 |
| 2777 | Seq. ID No. 5847 | Seq. ID No. 5848 |
| 2778 | Seq. ID No. 5849 | Seq. ID No. 5850 |
| 2779 | Seq. ID No. 5851 | Seq. ID No. 5852 |
| 2780 | Seq. ID No. 5853 | Seq. ID No. 5854 |
| 2784 | Seq. ID No. 5855 | Seq. ID No. 5856 |
| 2785b | Seq. ID No. 5859 | Seq. ID No. 5860 |
| 2786c | Seq. ID No. 5865 | Seq. ID No. 5866 |
| 2787c | Seq. ID No. 5871 | Seq. ID No. 5872 |
| 2789 | Seq. ID No. 5875 | Seq. ID No. 5876 |
| 2790 | Seq. ID No. 5877 | Seq. ID No. 5878 |
| 2793 | Seq. ID No. 5881 | Seq. ID No. 5882 |
| 2798 | Seq. ID No. 5887 | Seq. ID No. 5888 |
| 2799b | Seq. ID No. 5891 | Seq. ID No. 5892 |
| 2801b | Seq. ID No. 5895 | Seq. ID No. 5896 |
| 2804 | Seq. ID No. 5897 | Seq. ID No. 5898 |
| 2805b | Seq. ID No. 5901 | Seq. ID No. 5902 |
| 2809 | Seq. ID No. 5903 | Seq. ID No. 5904 |
| 2810d | Seq. ID No. 5911 | Seq. ID No. 5912 |
| 2812 | Seq. ID No. 5913 | Seq. ID No. 5914 |
| 2814 | Seq. ID No. 5915 | Seq. ID No. 5916 |
| 2815 | Seq. ID No. 5917 | Seq. ID No. 5918 |
| 2816 | Seq. ID No. 5919 | Seq. ID No. 5920 |
| 2818e | Seq. ID No. 5929 | Seq. ID No. 5930 |
| 2819 | Seq. ID No. 5931 | Seq. ID No. 5932 |
| 2820 | Seq. ID No. 5933 | Seq. ID No. 5934 |
| 2821b | Seq. ID No. 5937 | Seq. ID No. 5938 |
| 2822b | Seq. ID No. 5941 | Seq. ID No. 5942 |
| 2823 | Seq. ID No. 5943 | Seq. ID No. 5944 |
| 2824b | Seq. ID No. 5947 | Seq. ID No. 5948 |
| 2829b | Seq. ID No. 5951 | Seq. ID No. 5952 |
| 2831b | Seq. ID No. 5953 | Seq. ID No. 5954 |
| 2832 | Seq. ID No. 5955 | Seq. ID No. 5956 |
| 2833 | Seq. ID No. 5957 | Seq. ID No. 5958 |
| 2834b | Seq. ID No. 5961 | Seq. ID No. 5962 |
| 2842b | Seq. ID No. 5969 | Seq. ID No. 5970 |
| 2843b | Seq. ID No. 5973 | Seq. ID No. 5974 |
| 2846b | Seq. ID No. 5979 | Seq. ID No. 5980 |
| 2848 | Seq. ID No. 5981 | Seq. ID No. 5982 |
| 2851 | Seq. ID No. 5985 | Seq. ID No. 5986 |
| 2853 | Seq. ID No. 5989 | Seq. ID No. 5990 |
| 2854b | Seq. ID No. 5993 | Seq. ID No. 5994 |
| 2856c | Seq. ID No. 6001 | Seq. ID No. 6002 |
| 2857 | Seq. ID No. 6003 | Seq. ID No. 6004 |
| 2860c | Seq. ID No. 6011 | Seq. ID No. 6012 |
| 2863 | Seq. ID No. 6015 | Seq. ID No. 6016 |
| 2864 | Seq. ID No. 6017 | Seq. ID No. 6018 |
| 2868b | Seq. ID No. 6021 | Seq. ID No. 6022 |
| 2869c | Seq. ID No. 6027 | Seq. ID No. 6028 |
| 2871 | Seq. ID No. 6029 | Seq. ID No. 6030 |
| 2875 | Seq. ID No. 6033 | Seq. ID No. 6034 |
| 2877 | Seq. ID No. 6035 | Seq. ID No. 6036 |
| 2879c | Seq. ID No. 6041 | Seq. ID No. 6042 |
| 2881 | Seq. ID No. 6043 | Seq. ID No. 6044 |
| 2883d | Seq. ID No. 6053 | Seq. ID No. 6054 |
| 2886b | Seq. ID No. 6057 | Seq. ID No. 6058 |
| 2890 | Seq. ID No. 6065 | Seq. ID No. 6066 |
| 2891c | Seq. ID No. 6071 | Seq. ID No. 6072 |
| 2894c | Seq. ID No. 6079 | Seq. ID No. 6080 |
| 2897b | Seq. ID No. 6085 | Seq. ID No. 6086 |
| 2900b | Seq. ID No. 6089 | Seq. ID No. 6090 |
| 2902 | Seq. ID No. 6095 | Seq. ID No. 6096 |
| 2904 | Seq. ID No. 6097 | Seq. ID No. 6098 |
| 2905b | Seq. ID No. 6101 | Seq. ID No. 6102 |
| 2906 | Seq. ID No. 6103 | Seq. ID No. 6104 |
| 2909d | Seq. ID No. 6111 | Seq. ID No. 6112 |
| 2912 | Seq. ID No. 6113 | Seq. ID No. 6114 |
| 2913b | Seq. ID No. 6117 | Seq. ID No. 6118 |
| 2915c | Seq. ID No. 6123 | Seq. ID No. 6124 |
| 2916 | Seq. ID No. 6125 | Seq. ID No. 6126 |
| 2918 | Seq. ID No. 6129 | Seq. ID No. 6130 |
| 2920 | Seq. ID No. 6131 | Seq. ID No. 6132 |
| 2921 | Seq. ID No. 6133 | Seq. ID No. 6134 |
| 2923c | Seq. ID No. 6141 | Seq. ID No. 6142 |
| 2925b | Seq. ID No. 6145 | Seq. ID No. 6146 |
| 2926b | Seq. ID No. 6149 | Seq. ID No. 6150 |
| 2928c | Seq. ID No. 6157 | Seq. ID No. 6158 |
| 2931c | Seq. ID No. 6165 | Seq. ID No. 6166 |
| 2933b | Seq. ID No. 6169 | Seq. ID No. 6170 |
| 2934 | Seq. ID No. 6171 | Seq. ID No. 6172 |
| 2935b | Seq. ID No. 6175 | Seq. ID No. 6176 |
| 2936 | Seq. ID No. 6177 | Seq. ID No. 6178 |
| 2939b | Seq. ID No. 6181 | Seq. ID No. 6182 |
| 2941c | Seq. ID No. 6187 | Seq. ID No. 6188 |
| 2943d | Seq. ID No. 6195 | Seq. ID No. 6196 |
| 2947 | Seq. ID No. 6201 | Seq. ID No. 6202 |
| 2948d | Seq. ID No. 6209 | Seq. ID No. 6210 |
| 2950b | Seq. ID No. 6213 | Seq. ID No. 6214 |
| 2951 | Seq. ID No. 6215 | Seq. ID No. 6216 |
| 2954 | Seq. ID No. 6219 | Seq. ID No. 6220 |
| 2955g | Seq. ID No. 6233 | Seq. ID No. 6234 |
| 2957 | Seq. ID No. 6235 | Seq. ID No. 6236 |
| 2959 | Seq. ID No. 6243 | Seq. ID No. 6244 |
| 2962b | Seq. ID No. 6249 | Seq. ID No. 6250 |
| 2963 | Seq. ID No. 6251 | Seq. ID No. 6252 |
| 2965c | Seq. ID No. 6257 | Seq. ID No. 6258 |
| 2967 | Seq. ID No. 6261 | Seq. ID No. 6262 |
| 2969 | Seq. ID No. 6263 | Seq. ID No. 6264 |
| 2971b | Seq. ID No. 6271 | Seq. ID No. 6272 |
| 2974b | Seq. ID No. 6275 | Seq. ID No. 6276 |
| 2975 | Seq. ID No. 6277 | Seq. ID No. 6278 |
| 2977b | Seq. ID No. 6281 | Seq. ID No. 6282 |
| 2979 | Seq. ID No. 6287 | Seq. ID No. 6288 |
| 2980e | Seq. ID No. 6297 | Seq. ID No. 6298 |
| 2984 | Seq. ID No. 6299 | Seq. ID No. 6300 |
| 2986c | Seq. ID No. 6305 | Seq. ID No. 6306 |
| 2988c | Seq. ID No. 6311 | Seq. ID No. 6312 |
| 2990 | Seq. ID No. 6315 | Seq. ID No. 6316 |
| 2991c | Seq. ID No. 6321 | Seq. ID No. 6322 |
| 2992 | Seq. ID No. 6323 | Seq. ID No. 6324 |
| 2993b | Seq. ID No. 6327 | Seq. ID No. 6328 |
| 2995b | Seq. ID No. 6331 | Seq. ID No. 6332 |
| 2997 | Seq. ID No. 6333 | Seq. ID No. 6334 |
| 2998b | Seq. ID No. 6337 | Seq. ID No. 6338 |
| 2999 | Seq. ID No. 6339 | Seq. ID No. 6340 |
| 3001 | Seq. ID No. 6341 | Seq. ID No. 6342 |
| 3003 | Seq. ID No. 6343 | Seq. ID No. 6344 |
| 3005 | Seq. ID No. 6345 | Seq. ID No. 6346 |
| 3008d | Seq. ID No. 6353 | Seq. ID No. 6354 |
| 3010b | Seq. ID No. 6357 | Seq. ID No. 6358 |
| 3012 | Seq. ID No. 6359 | Seq. ID No. 6360 |
| 3015 | Seq. ID No. 6361 | Seq. ID No. 6362 |
| 3016c | Seq. ID No. 6367 | Seq. ID No. 6368 |
| 3017 | Seq. ID No. 6369 | Seq. ID No. 6370 |
| 3018b | Seq. ID No. 6373 | Seq. ID No. 6374 |
| 3020 | Seq. ID No. 6375 | Seq. ID No. 6376 |
| 3021 | Seq. ID No. 6377 | Seq. ID No. 6378 |
| 3022 | Seq. ID No. 6379 | Seq. ID No. 6380 |
| 3026b | Seq. ID No. 6389 | Seq. ID No. 6390 |
| 3028c | Seq. ID No. 6395 | Seq. ID No. 6396 |
| 3029b | Seq. ID No. 6399 | Seq. ID No. 6400 |
| 3031b | Seq. ID No. 6403 | Seq. ID No. 6404 |
| 3032b | Seq. ID No. 6407 | Seq. ID No. 6408 |
| 3033b | Seq. ID No. 6411 | Seq. ID No. 6412 |
| 3035 | Seq. ID No. 6413 | Seq. ID No. 6414 |
| 3037b | Seq. ID No. 6417 | Seq. ID No. 6418 |
| 3040b | Seq. ID No. 6423 | Seq. ID No. 6424 |
| 3044 | Seq. ID No. 6433 | Seq. ID No. 6434 |
| 3045b | Seq. ID No. 6437 | Seq. ID No. 6438 |
| 3048a | Seq. ID No. 6439 | Seq. ID No. 6440 |
| 3049c | Seq. ID No. 6445 | Seq. ID No. 6446 |
| 3051b | Seq. ID No. 6449 | Seq. ID No. 6450 |
| 3052 | Seq. ID No. 6451 | Seq. ID No. 6452 |
| 3055b | Seq. ID No. 6457 | Seq. ID No. 6458 |
| 3057 | Seq. ID No. 6459 | Seq. ID No. 6460 |
| 3059 | Seq. ID No. 6461 | Seq. ID No. 6462 |
| 3060 | Seq. ID No. 6463 | Seq. ID No. 6464 |
| 3065b | Seq. ID No. 6477 | Seq. ID No. 6478 |
| 3067 | Seq. ID No. 6481 | Seq. ID No. 6482 |
| 3068 | Seq. ID No. 6483 | Seq. ID No. 6484 |
| 3071 | Seq. ID No. 6487 | Seq. ID No. 6488 |
| 3072a | Seq. ID No. 6489 | Seq. ID No. 6490 |
| 3073 | Seq. ID No. 6491 | Seq. ID No. 6492 |
| 3074 | Seq. ID No. 6493 | Seq. ID No. 6494 |
| 3075 | Seq. ID No. 6495 | Seq. ID No. 6496 |
| 3077c | Seq. ID No. 6503 | Seq. ID No. 6504 |
| 3078 | Seq. ID No. 6505 | Seq. ID No. 6506 |
| 3081b | Seq. ID No. 6511 | Seq. ID No. 6512 |
| 3083d | Seq. ID No. 6515 | Seq. ID No. 6516 |
| 3086 | Seq. ID No. 6519 | Seq. ID No. 6520 |
| 3087c | Seq. ID No. 6523 | Seq. ID No. 6524 |
| 3088 | Seq. ID No. 6525 | Seq. ID No. 6526 |
| 3089b | Seq. ID No. 6529 | Seq. ID No. 6530 |
| 3090 | Seq. ID No. 6531 | Seq. ID No. 6532 |
| 3091 | Seq. ID No. 6533 | Seq. ID No. 6534 |
| 3092b | Seq. ID No. 6537 | Seq. ID No. 6538 |
| 3097 | Seq. ID No. 6545 | Seq. ID No. 6546 |
| 3098c | Seq. ID No. 6551 | Seq. ID No. 6552 |
| 3099 | Seq. ID No. 6553 | Seq. ID No. 6554 |
| 3101 | Seq. ID No. 6559 | Seq. ID No. 6560 |
| 3103b | Seq. ID No. 6563 | Seq. ID No. 6564 |
| 3105 | Seq. ID No. 6567 | Seq. ID No. 6568 |
| 3108b | Seq. ID No. 6577 | Seq. ID No. 6578 |
| 3109 | Seq. ID No. 6579 | Seq. ID No. 6580 |
| 3110b | Seq. ID No. 6583 | Seq. ID No. 6584 |
| 3113 | Seq. ID No. 6585 | Seq. ID No. 6586 |
| 3116 | Seq. ID No. 6587 | Seq. ID No. 6588 |
| 3118a | Seq. ID No. 6589 | Seq. ID No. 6590 |
| 3119f | Seq. ID No. 6603 | Seq. ID No. 6604 |
| 3121 | Seq. ID No. 6605 | Seq. ID No. 6606 |
| 3122b | Seq. ID No. 6609 | Seq. ID No. 6610 |
| 3123b | Seq. ID No. 6613 | Seq. ID No. 6614 |
| 3124 | Seq. ID No. 6615 | Seq. ID No. 6616 |
| 3126 | Seq. ID No. 6617 | Seq. ID No. 6618 |
| 3129b | Seq. ID No. 6621 | Seq. ID No. 6622 |
| 3131 | Seq. ID No. 6623 | Seq. ID No. 6624 |
| 3134b | Seq. ID No. 6627 | Seq. ID No. 6628 |
| 3138 | Seq. ID No. 6629 | Seq. ID No. 6630 |
| 3141 | Seq. ID No. 6633 | Seq. ID No. 6634 |
| 3145c | Seq. ID No. 6639 | Seq. ID No. 6640 |

Listed in Table 19 are the 1534 ORFs detected by the GeneMark™ ORF finder program trained on Bacillus subtilis published ORFs.

TABLE 19
GENEMARK ™ ORFs

| ORF Number | DNA SEQ ID Number | Protein SEQ ID Number |
|---|---|---|
| 1 | Seq. ID No. 1 | Seq. ID No. 2 |
| 2a | Seq. ID No. 3 | Seq. ID No. 4 |
| 4c | Seq. ID No. 11 | Seq. ID No. 12 |
| 5 | Seq. ID No. 13 | Seq. ID No. 14 |
| 7 | Seq. ID No. 15 | Seq. ID No. 16 |
| 10 | Seq. ID No. 17 | Seq. ID No. 18 |
| 17 | Seq. ID No. 25 | Seq. ID No. 26 |
| 18 | Seq. ID No. 27 | Seq. ID No. 28 |
| 20 | Seq. ID No. 29 | Seq. ID No. 30 |
| 23b | Seq. ID No. 35 | Seq. ID No. 36 |
| 24 | Seq. ID No. 37 | Seq. ID No. 38 |
| 32 | Seq. ID No. 39 | Seq. ID No. 40 |
| 36 | Seq. ID No. 41 | Seq. ID No. 42 |
| 37 | Seq. ID No. 43 | Seq. ID No. 44 |
| 38a | Seq. ID No. 45 | Seq. ID No. 46 |
| 39a | Seq. ID No. 49 | Seq. ID No. 50 |
| 40 | Seq. ID No. 53 | Seq. ID No. 54 |
| 41b | Seq. ID No. 57 | Seq. ID No. 58 |
| 43b | Seq. ID No. 61 | Seq. ID No. 62 |
| 45 | Seq. ID No. 69 | Seq. ID No. 70 |
| 46a | Seq. ID No. 71 | Seq. ID No. 72 |
| 47 | Seq. ID No. 75 | Seq. ID No. 76 |
| 48c | Seq. ID No. 81 | Seq. ID No. 82 |
| 50 | Seq. ID No. 83 | Seq. ID No. 84 |
| 51c | Seq. ID No. 89 | Seq. ID No. 90 |
| 57a | Seq. ID No. 95 | Seq. ID No. 96 |
| 60a | Seq. ID No. 101 | Seq. ID No. 102 |
| 62c | Seq. ID No. 111 | Seq. ID No. 112 |
| 65b | Seq. ID No. 119 | Seq. ID No. 120 |
| 67 | Seq. ID No. 125 | Seq. ID No. 126 |
| 71 | Seq. ID No. 129 | Seq. ID No. 130 |
| 74 | Seq. ID No. 135 | Seq. ID No. 136 |
| 77 | Seq. ID No. 137 | Seq. ID No. 138 |
| 80a | Seq. ID No. 141 | Seq. ID No. 142 |
| 81a | Seq. ID No. 147 | Seq. ID No. 148 |
| 83 | Seq. ID No. 151 | Seq. ID No. 152 |
| 85b | Seq. ID No. 155 | Seq. ID No. 156 |
| 88 | Seq. ID No. 167 | Seq. ID No. 168 |
| 91a | Seq. ID No. 171 | Seq. ID No. 172 |
| 93b | Seq. ID No. 181 | Seq. ID No. 182 |
| 94 | Seq. ID No. 185 | Seq. ID No. 186 |
| 95b | Seq. ID No. 187 | Seq. ID No. 188 |
| 97b | Seq. ID No. 189 | Seq. ID No. 190 |
| 99 | Seq. ID No. 191 | Seq. ID No. 192 |
| 102 | Seq. ID No. 195 | Seq. ID No. 196 |
| 104b | Seq. ID No. 199 | Seq. ID No. 200 |
| 106 | Seq. ID No. 207 | Seq. ID No. 208 |
| 109c | Seq. ID No. 213 | Seq. ID No. 214 |
| 111b | Seq. ID No. 219 | Seq. ID No. 220 |
| 112a | Seq. ID No. 221 | Seq. ID No. 222 |
| 114 | Seq. ID No. 225 | Seq. ID No. 226 |
| 115 | Seq. ID No. 227 | Seq. ID No. 228 |
| 119a | Seq. ID No. 229 | Seq. ID No. 230 |
| 121 | Seq. ID No. 233 | Seq. ID No. 234 |
| 124c | Seq. ID No. 239 | Seq. ID No. 240 |
| 125 | Seq. ID No. 241 | Seq. ID No. 242 |
| 127a | Seq. ID No. 243 | Seq. ID No. 244 |
| 129c | Seq. ID No. 253 | Seq. ID No. 254 |
| 134 | Seq. ID No. 261 | Seq. ID No. 262 |
| 135a | Seq. ID No. 263 | Seq. ID No. 264 |
| 137a | Seq. ID No. 267 | Seq. ID No. 268 |
| 138b | Seq. ID No. 273 | Seq. ID No. 274 |
| 142 | Seq. ID No. 275 | Seq. ID No. 276 |
| 143 | Seq. ID No. 277 | Seq. ID No. 278 |
| 144a | Seq. ID No. 279 | Seq. ID No. 280 |
| 145d | Seq. ID No. 291 | Seq. ID No. 292 |
| 147 | Seq. ID No. 293 | Seq. ID No. 294 |
| 151a | Seq. ID No. 295 | Seq. ID No. 296 |
| 152a | Seq. ID No. 299 | Seq. ID No. 300 |
| 153a | Seq. ID No. 303 | Seq. ID No. 304 |
| 154 | Seq. ID No. 309 | Seq. ID No. 310 |
| 155a | Seq. ID No. 311 | Seq. ID No. 312 |
| 157a | Seq. ID No. 315 | Seq. ID No. 316 |
| 158a | Seq. ID No. 319 | Seq. ID No. 320 |
| 160a | Seq. ID No. 323 | Seq. ID No. 324 |
| 162c | Seq. ID No. 331 | Seq. ID No. 332 |
| 165a | Seq. ID No. 341 | Seq. ID No. 342 |
| 167 | Seq. ID No. 345 | Seq. ID No. 346 |
| 171a | Seq. ID No. 351 | Seq. ID No. 352 |
| 172 | Seq. ID No. 355 | Seq. ID No. 356 |
| 175 | Seq. ID No. 361 | Seq. ID No. 362 |
| 176 | Seq. ID No. 363 | Seq. ID No. 364 |
| 177a | Seq. ID No. 365 | Seq. ID No. 366 |
| 180b | Seq. ID No. 375 | Seq. ID No. 376 |
| 184 | Seq. ID No. 379 | Seq. ID No. 380 |
| 185 | Seq. ID No. 381 | Seq. ID No. 382 |
| 186 | Seq. ID No. 383 | Seq. ID No. 384 |
| 188 | Seq. ID No. 389 | Seq. ID No. 390 |
| 189b | Seq. ID No. 391 | Seq. ID No. 392 |
| 192a | Seq. ID No. 395 | Seq. ID No. 396 |
| 193 | Seq. ID No. 399 | Seq. ID No. 400 |
| 195 | Seq. ID No. 405 | Seq. ID No. 406 |
| 197a | Seq. ID No. 409 | Seq. ID No. 410 |
| 198a | Seq. ID No. 413 | Seq. ID No. 414 |
| 200 | Seq. ID No. 417 | Seq. ID No. 418 |
| 202 | Seq. ID No. 421 | Seq. ID No. 422 |
| 203 | Seq. ID No. 423 | Seq. ID No. 424 |
| 204 | Seq. ID No. 425 | Seq. ID No. 426 |
| 205 | Seq. ID No. 427 | Seq. ID No. 428 |
| 206 | Seq. ID No. 429 | Seq. ID No. 430 |
| 208b | Seq. ID No. 435 | Seq. ID No. 436 |
| 210 | Seq. ID No. 437 | Seq. ID No. 438 |
| 211 | Seq. ID No. 439 | Seq. ID No. 440 |
| 213 | Seq. ID No. 441 | Seq. ID No. 442 |
| 215a | Seq. ID No. 443 | Seq. ID No. 444 |
| 218 | Seq. ID No. 445 | Seq. ID No. 446 |
| 219 | Seq. ID No. 447 | Seq. ID No. 448 |
| 220a | Seq. ID No. 449 | Seq. ID No. 450 |
| 226a | Seq. ID No. 459 | Seq. ID No. 460 |
| 228a | Seq. ID No. 465 | Seq. ID No. 466 |
| 236c | Seq. ID No. 485 | Seq. ID No. 486 |
| 245c | Seq. ID No. 501 | Seq. ID No. 502 |
| 246b | Seq. ID No. 505 | Seq. ID No. 506 |
| 248b | Seq. ID No. 511 | Seq. ID No. 512 |
| 249a | Seq. ID No. 513 | Seq. ID No. 514 |
| 251 | Seq. ID No. 517 | Seq. ID No. 518 |
| 253a | Seq. ID No. 519 | Seq. ID No. 520 |
| 254 | Seq. ID No. 523 | Seq. ID No. 524 |
| 255c | Seq. ID No. 529 | Seq. ID No. 530 |
| 258 | Seq. ID No. 533 | Seq. ID No. 534 |
| 259a | Seq. ID No. 535 | Seq. ID No. 536 |
| 260 | Seq. ID No. 541 | Seq. ID No. 542 |
| 262 | Seq. ID No. 543 | Seq. ID No. 544 |
| 265 | Seq. ID No. 551 | Seq. ID No. 552 |
| 267b | Seq. ID No. 553 | Seq. ID No. 554 |
| 269a | Seq. ID No. 559 | Seq. ID No. 560 |
| 271a | Seq. ID No. 563 | Seq. ID No. 564 |
| 273b | Seq. ID No. 569 | Seq. ID No. 570 |
| 277c | Seq. ID No. 575 | Seq. ID No. 576 |
| 279b | Seq. ID No. 579 | Seq. ID No. 580 |
| 280b | Seq. ID No. 583 | Seq. ID No. 584 |
| 283a | Seq. ID No. 591 | Seq. ID No. 592 |
| 284a | Seq. ID No. 595 | Seq. ID No. 596 |
| 286a | Seq. ID No. 601 | Seq. ID No. 602 |
| 287 | Seq. ID No. 605 | Seq. ID No. 606 |
| 290a | Seq. ID No. 607 | Seq. ID No. 608 |
| 293 | Seq. ID No. 611 | Seq. ID No. 612 |
| 294 | Seq. ID No. 613 | Seq. ID No. 614 |
| 296 | Seq. ID No. 615 | Seq. ID No. 616 |
| 301a | Seq. ID No. 621 | Seq. ID No. 622 |
| 304b | Seq. ID No. 627 | Seq. ID No. 628 |
| 307 | Seq. ID No. 629 | Seq. ID No. 630 |
| 309 | Seq. ID No. 633 | Seq. ID No. 634 |
| 310 | Seq. ID No. 635 | Seq. ID No. 636 |
| 311 | Seq. ID No. 637 | Seq. ID No. 638 |
| 313c | Seq. ID No. 643 | Seq. ID No. 644 |
| 315 | Seq. ID No. 649 | Seq. ID No. 650 |
| 316a | Seq. ID No. 651 | Seq. ID No. 652 |
| 317b | Seq. ID No. 655 | Seq. ID No. 656 |
| 318a | Seq. ID No. 657 | Seq. ID No. 658 |
| 321 | Seq. ID No. 669 | Seq. ID No. 670 |
| 323a | Seq. ID No. 671 | Seq. ID No. 672 |
| 324 | Seq. ID No. 675 | Seq. ID No. 676 |
| 327c | Seq. ID No. 681 | Seq. ID No. 682 |
| 329 | Seq. ID No. 683 | Seq. ID No. 684 |
| 330a | Seq. ID No. 685 | Seq. ID No. 686 |
| 332 | Seq. ID No. 693 | Seq. ID No. 694 |
| 337 | Seq. ID No. 703 | Seq. ID No. 704 |
| 338a | Seq. ID No. 705 | Seq. ID No. 706 |
| 341a | Seq. ID No. 711 | Seq. ID No. 712 |
| 342f | Seq. ID No. 725 | Seq. ID No. 726 |
| 344a | Seq. ID No. 731 | Seq. ID No. 732 |
| 345 | Seq. ID No. 735 | Seq. ID No. 736 |
| 346a | Seq. ID No. 737 | Seq. ID No. 738 |
| 347 | Seq. ID No. 741 | Seq. ID No. 742 |
| 349a | Seq. ID No. 743 | Seq. ID No. 744 |
| 350a | Seq. ID No. 749 | Seq. ID No. 750 |
| 351 | Seq. ID No. 753 | Seq. ID No. 754 |
| 352c | Seq. ID No. 759 | Seq. ID No. 760 |
| 356c | Seq. ID No. 773 | Seq. ID No. 774 |
| 358c | Seq. ID No. 781 | Seq. ID No. 782 |
| 362 | Seq. ID No. 783 | Seq. ID No. 784 |
| 365a | Seq. ID No. 785 | Seq. ID No. 786 |
| 366b | Seq. ID No. 789 | Seq. ID No. 790 |
| 367a | Seq. ID No. 793 | Seq. ID No. 794 |
| 371a | Seq. ID No. 797 | Seq. ID NO. 798 |
| 372b | Seq. ID No. 803 | Seq. ID No. 804 |
| 378a | Seq. ID No. 815 | Seq. ID No. 816 |
| 382b | Seq. ID No. 819 | Seq. ID No. 820 |
| 383a | Seq. ID No. 821 | Seq. ID No. 822 |
| 384b | Seq. ID No. 827 | Seq. ID No. 828 |
| 385b | Seq. ID No. 831 | Seq. ID No. 832 |
| 386 | Seq. ID No. 833 | Seq. ID No. 834 |
| 387 | Seq. ID No. 835 | Seq. ID No. 836 |
| 390 | Seq. ID No. 837 | Seq. ID No. 838 |
| 393b | Seq. ID No. 841 | Seq. ID No. 842 |
| 395a | Seq. ID No. 843 | Seq. ID No. 844 |
| 396 | Seq. ID No. 849 | Seq. ID No. 850 |
| 398 | Seq. ID No. 851 | Seq. ID No. 852 |
| 400a | Seq. ID No. 853 | Seq. ID No. 854 |
| 401a | Seq. ID No. 857 | Seq. ID No. 858 |
| 403c | Seq. ID No. 865 | Seq. ID No. 866 |
| 406a | Seq. ID No. 869 | Seq. ID No. 870 |
| 409a | Seq. ID No. 873 | Seq. ID No. 874 |
| 410a | Seq. ID No. 877 | Seq. ID No. 878 |
| 413a | Seq. ID No. 883 | Seq. ID No. 884 |
| 416a | Seq. ID No. 887 | Seq. ID No. 888 |
| 417 | Seq. ID No. 891 | Seq. ID No. 892 |
| 418b | Seq. ID No. 895 | Seq. ID No. 896 |
| 419c | Seq. ID No. 901 | Seq. ID No. 902 |
| 421 | Seq. ID No. 907 | Seq. ID No. 908 |
| 424a | Seq. ID No. 911 | Seq. ID No. 912 |
| 426a | Seq. ID No. 917 | Seq. ID No. 918 |
| 427a | Seq. ID No. 921 | Seq. ID No. 922 |
| 429a | Seq. ID No. 927 | Seq. ID No. 928 |
| 431 | Seq. ID No. 935 | Seq. ID No. 936 |
| 435 | Seq. ID No. 939 | Seq. ID No. 940 |
| 438a | Seq. ID No. 945 | Seq. ID No. 946 |
| 440 | Seq. ID No. 951 | Seq. ID No. 952 |
| 441a | Seq. ID No. 953 | Seq. ID No. 954 |
| 444a | Seq. ID No. 959 | Seq. ID No. 960 |
| 445a | Seq. ID No. 963 | Seq. ID No. 964 |
| 448 | Seq. ID No. 967 | Seq. ID No. 968 |
| 450a | Seq. ID No. 969 | Seq. ID No. 970 |
| 452a | Seq. ID No. 977 | Seq. ID No. 978 |
| 455a | Seq. ID No. 989 | Seq. ID No. 990 |
| 458a | Seq. ID No. 995 | Seq. ID No. 996 |
| 460a | Seq. ID No. 999 | Seq. ID No. 1000 |
| 462 | Seq. ID No. 1005 | Seq. ID No. 1006 |
| 463a | Seq. ID No. 1007 | Seq. ID No. 1008 |
| 464c | Seq. ID No. 1015 | Seq. ID No. 1016 |
| 467a | Seq. ID No. 1021 | Seq. ID No. 1022 |
| 472d | Seq. ID No. 1035 | Seq. ID No. 1036 |
| 473 | Seq. ID No. 1041 | Seq. ID No. 1042 |
| 474 | Seq. ID No. 1043 | Seq. ID No. 1044 |
| 478a | Seq. ID No. 1049 | Seq. ID No. 1050 |
| 479 | Seq. ID No. 1053 | Seq. ID No. 1054 |
| 480b | Seq. ID No. 1057 | Seq. ID No. 1058 |
| 481a | Seq. ID No. 1059 | Seq. ID No. 1060 |
| 482a | Seq. ID No. 1063 | Seq. ID No. 1064 |
| 483 | Seq. ID No. 1067 | Seq. ID No. 1068 |
| 490 | Seq. ID No. 1069 | Seq. ID No. 1070 |
| 491a | Seq. ID No. 1071 | Seq. ID No. 1072 |
| 492 | Seq. ID No. 1075 | Seq. ID No. 1076 |
| 495c | Seq. ID No. 1083 | Seq. ID No. 1084 |
| 498a | Seq. ID No. 1085 | Seq. ID No. 1086 |
| 499a | Seq. ID No. 1089 | Seq. ID No. 1090 |
| 500 | Seq. ID No. 1093 | Seq. ID No. 1094 |
| 502c | Seq. ID No. 1097 | Seq. ID No. 1098 |
| 504 | Seq. ID No. 1099 | Seq. ID No. 1100 |
| 505a | Seq. ID No. 1101 | Seq. ID No. 1102 |
| 509 | Seq. ID No. 1109 | Seq. ID No. 1110 |
| 512a | Seq. ID No. 1113 | Seq. ID No. 1114 |
| 514 | Seq. ID No. 1117 | Seq. ID No. 1118 |
| 515b | Seq. ID No. 1121 | Seq. ID No. 1122 |
| 518c | Seq. ID No. 1129 | Seq. ID No. 1130 |
| 523 | Seq. ID No. 1133 | Seq. ID No. 1134 |
| 524b | Seq. ID No. 1137 | Seq. ID No. 1138 |
| 527a | Seq. ID No. 1141 | Seq. ID No. 1142 |
| 528a | Seq. ID No. 1145 | Seq. ID No. 1146 |
| 529 | Seq. ID No. 1149 | Seq. ID No. 1150 |
| 533 | Seq. ID No. 1155 | Seq. ID No. 1156 |
| 535 | Seq. ID No. 1157 | Seq. ID No. 1158 |
| 536 | Seq. ID No. 1159 | Seq. ID No. 1160 |
| 539c | Seq. ID No. 1163 | Seq. ID No. 1164 |
| 541d | Seq. ID No. 1171 | Seq. ID No. 1172 |
| 542 | Seq. ID No. 1173 | Seq. ID No. 1174 |
| 543a | Seq. ID No. 1175 | Seq. ID No. 1176 |
| 544 | Seq. ID No. 1181 | Seq. ID No. 1182 |
| 545b | Seq. ID No. 1185 | Seq. ID No. 1186 |
| 546a | Seq. ID No. 1189 | Seq. ID No. 1190 |
| 548 | Seq. ID No. 1199 | Seq. ID No. 1200 |
| 551 | Seq. ID No. 1205 | Seq. ID No. 1206 |
| 554a | Seq. ID No. 1209 | Seq. ID No. 1210 |
| 557a | Seq. ID No. 1215 | Seq. ID No. 1216 |
| 560a | Seq. ID No. 1219 | Seq. ID No. 1220 |
| 561 | Seq. ID No. 1223 | Seq. ID No. 1224 |
| 563 | Seq. ID No. 1225 | Seq. ID No. 1226 |
| 569 | Seq. ID No. 1233 | Seq. ID No. 1234 |
| 570a | Seq. ID No. 1235 | Seq. ID No. 1236 |
| 573 | Seq. ID No. 1241 | Seq. ID No. 1242 |
| 580a | Seq. ID No. 1251 | Seq. ID No. 1252 |
| 583a | Seq. ID No. 1255 | Seq. ID No. 1256 |
| 586a | Seq. ID No. 1261 | Seq. ID No. 1262 |
| 587d | Seq. ID No. 1271 | Seq. ID No. 1272 |
| 590 | Seq. ID No. 1281 | Seq. ID No. 1282 |
| 609a | Seq. ID No. 1315 | Seq. ID No. 1316 |
| 611 | Seq. ID No. 1323 | Seq. ID No. 1324 |
| 613d | Seq. ID No. 1331 | Seq. ID No. 1332 |
| 614a | Seq. ID No. 1333 | Seq. ID No. 1334 |
| 618 | Seq. ID No. 1339 | Seq. ID No. 1340 |
| 620 | Seq. ID No. 1345 | Seq. ID No. 1346 |
| 623 | Seq. ID No. 1347 | Seq. ID No. 1348 |
| 625 | Seq. ID No. 1355 | Seq. ID No. 1356 |
| 626a | Seq. ID No. 1357 | Seq. ID No. 1358 |
| 631d | Seq. ID No. 1369 | Seq. ID No. 1370 |
| 633a | Seq. ID No. 1373 | Seq. ID No. 1374 |
| 637c | Seq. ID No. 1387 | Seq. ID No. 1388 |
| 641a | Seq. ID No. 1391 | Seq. ID No. 1392 |
| 643 | Seq. ID No. 1395 | Seq. ID No. 1396 |
| 644a | Seq. ID No. 1397 | Seq. ID No. 1398 |
| 650 | Seq. ID No. 1403 | Seq. ID No. 1404 |
| 652 | Seq. ID No. 1405 | Seq. ID No. 1406 |
| 653a | Seq. ID No. 1407 | Seq. ID No. 1408 |
| 656 | Seq. ID No. 1413 | Seq. ID No. 1414 |
| 657c | Seq. ID No. 1415 | Seq. ID No. 1416 |
| 658a | Seq. ID No. 1417 | Seq. ID No. 1418 |
| 661 | Seq. ID No. 1425 | Seq. ID No. 1426 |
| 663 | Seq. ID No. 1427 | Seq. ID No. 1428 |
| 665 | Seq. ID No. 1429 | Seq. ID No. 1430 |
| 666 | Seq. ID No. 1431 | Seq. ID No. 1432 |
| 670 | Seq. ID No. 1433 | Seq. ID No. 1434 |
| 674a | Seq. ID No. 1435 | Seq. ID No. 1436 |
| 676 | Seq. ID No. 1439 | Seq. ID No. 1440 |
| 677a | Seq. ID No. 1441 | Seq. ID No. 1442 |
| 681 | Seq. ID No. 1451 | Seq. ID No. 1452 |
| 683a | Seq. ID No. 1453 | Seq. ID No. 1454 |
| 687 | Seq. ID No. 1459 | Seq. ID No. 1460 |
| 688a | Seq. ID No. 1461 | Seq. ID No. 1462 |
| 691b | Seq. ID No. 1469 | Seq. ID No. 1470 |
| 692 | Seq. ID No. 1471 | Seq. ID No. 1472 |
| 694 | Seq. ID No. 1473 | Seq. ID No. 1474 |
| 696 | Seq. ID No. 1475 | Seq. ID No. 1476 |
| 698 | Seq. ID No. 1477 | Seq. ID No. 1478 |
| 700b | Seq. ID No. 1483 | Seq. ID No. 1484 |
| 703 | Seq. ID No. 1487 | Seq. ID No. 1488 |
| 705a | Seq. ID No. 1489 | Seq. ID No. 1490 |
| 707 | Seq. ID No. 1493 | Seq. ID No. 1494 |
| 709 | Seq. ID No. 1495 | Seq. ID No. 1496 |
| 710 | Seq. ID No. 1497 | Seq. ID No. 1498 |
| 712b | Seq. ID No. 1501 | Seq. ID No. 1502 |
| 715a | Seq. ID No. 1503 | Seq. ID No. 1504 |
| 717 | Seq. ID No. 1507 | Seq. ID No. 1508 |
| 718a | Seq. ID No. 1509 | Seq. ID No. 1510 |
| 722c | Seq. ID No. 1517 | Seq. ID No. 1518 |
| 724b | Seq. ID No. 1521 | Seq. ID No. 1522 |
| 726 | Seq. ID No. 1527 | Seq. ID No. 1528 |
| 728b | Seq. ID No. 1531 | Seq. ID No. 1532 |
| 730c | Seq. ID No. 1537 | Seq. ID No. 1538 |
| 731b | Seq. ID No. 1541 | Seq. ID No. 1542 |
| 732 | Seq. ID No. 1543 | Seq. ID No. 1544 |
| 738c | Seq. ID No. 1549 | Seq. ID No. 1550 |
| 743b | Seq. ID No. 1561 | Seq. ID No. 1562 |
| 744 | Seq. ID No. 1563 | Seq. ID No. 1564 |
| 745a | Seq. ID No. 1565 | Seq. ID No. 1566 |
| 746a | Seq. ID No. 1569 | Seq. ID No. 1570 |
| 747 | Seq. ID No. 1573 | Seq. ID No. 1574 |
| 748a | Seq. ID No. 1575 | Seq. ID No. 1576 |
| 751 | Seq. ID No. 1581 | Seq. ID No. 1582 |
| 754c | Seq. ID No. 1587 | Seq. ID No. 1588 |
| 756a | Seq. ID No. 1591 | Seq. ID No. 1592 |
| 758 | Seq. ID No. 1595 | Seq. ID No. 1596 |
| 760 | Seq. ID No. 1597 | Seq. ID No. 1598 |
| 763a | Seq. ID No. 1605 | Seq. ID No. 1606 |
| 764 | Seq. ID No. 1609 | Seq. ID No. 1610 |
| 767 | Seq. ID No. 1613 | Seq. ID No. 1614 |
| 770c | Seq. ID No. 1619 | Seq. ID No. 1620 |
| 772b | Seq. ID No. 1623 | Seq. ID No. 1624 |
| 775 | Seq. ID No. 1627 | Seq. ID No. 1628 |
| 777a | Seq. ID No. 1629 | Seq. ID No. 1630 |
| 780a | Seq. ID No. 1635 | Seq. ID No. 1636 |
| 785 | Seq. ID No. 1641 | Seq. ID No. 1642 |
| 788b | Seq. ID No. 1647 | Seq. ID No. 1648 |
| 789b | Seq. ID No. 1651 | Seq. ID No. 1652 |
| 792 | Seq. ID No. 1657 | Seq. ID No. 1658 |
| 794a | Seq. ID No. 1659 | Seq. ID No. 1660 |
| 795 | Seq. ID No. 1667 | Seq. ID No. 1668 |
| 797 | Seq. ID No. 1669 | Seq. ID No. 1670 |
| 799e | Seq. ID No. 1679 | Seq. ID No. 1680 |
| 803a | Seq. ID No. 1683 | Seq. ID No. 1684 |
| 804a | Seq. ID No. 1687 | Seq. ID No. 1688 |
| 807 | Seq. ID No. 1691 | Seq. ID No. 1692 |
| 812 | Seq. ID No. 1697 | Seq. ID No. 1698 |
| 813 | Seq. ID No. 1699 | Seq. ID No. 1700 |
| 816 | Seq. ID No. 1701 | Seq. ID No. 1702 |
| 826 | Seq. ID No. 1707 | Seq. ID No. 1708 |
| 829a | Seq. ID No. 1709 | Seq. ID No. 1710 |
| 830 | Seq. ID No. 1713 | Seq. ID No. 1714 |
| 834b | Seq. ID No. 1717 | Seq. ID No. 1718 |
| 835a | Seq. ID No. 1721 | Seq. ID No. 1722 |
| 836a | Seq. ID No. 1725 | Seq. ID No. 1726 |
| 837c | Seq. ID No. 1735 | Seq. ID No. 1736 |
| 843 | Seq. ID No. 1737 | Seq. ID No. 1738 |
| 847a | Seq. ID No. 1739 | Seq. ID No. 1740 |
| 848 | Seq. ID No. 1743 | Seq. ID No. 1744 |
| 849a | Seq. ID No. 1745 | Seq. ID No. 1746 |
| 851a | Seq. ID No. 1749 | Seq. ID No. 1750 |
| 853b | Seq. ID No. 1755 | Seq. ID No. 1756 |
| 859c | Seq. ID No. 1769 | Seq. ID No. 1770 |
| 860 | Seq. ID No. 1773 | Seq. ID No. 1774 |
| 863 | Seq. ID No. 1777 | Seq. ID No. 1778 |
| 866b | Seq. ID No. 1785 | Seq. ID No. 1786 |
| 868b | Seq. ID No. 1789 | Seq. ID No. 1790 |
| 870 | Seq. ID No. 1791 | Seq. ID No. 1792 |
| 873c | Seq. ID No. 1797 | Seq. ID No. 1798 |
| 874a | Seq. ID No. 1799 | Seq. ID No. 1800 |
| 875 | Seq. ID No. 1803 | Seq. ID No. 1804 |
| 876 | Seq. ID No. 1805 | Seq. ID No. 1806 |
| 878a | Seq. ID No. 1807 | Seq. ID No. 1808 |
| 880c | Seq. ID No. 1817 | Seq. ID No. 1818 |
| 885 | Seq. ID No. 1819 | Seq. ID No. 1820 |
| 886 | Seq. ID No. 1821 | Seq. ID No. 1822 |
| 888 | Seq. ID No. 1825 | Seq. ID No. 1826 |
| 890a | Seq. ID No. 1827 | Seq. ID No. 1828 |
| 894 | Seq. ID No. 1831 | Seq. ID No. 1832 |
| 907a | Seq. ID No. 1835 | Seq. ID No. 1836 |
| 908b | Seq. ID No. 1841 | Seq. ID No. 1842 |
| 911 | Seq. ID No. 1845 | Seq. ID No. 1846 |
| 912 | Seq. ID No. 1847 | Seq. ID No. 1848 |
| 916 | Seq. ID No. 1849 | Seq. ID No. 1850 |
| 917 | Seq. ID No. 1851 | Seq. ID No. 1852 |
| 918c | Seq. ID No. 1855 | Seq. ID No. 1856 |
| 923 | Seq. ID No. 1859 | Seq. ID No. 1860 |
| 925a | Seq. ID No. 1861 | Seq. ID No. 1862 |
| 926 | Seq. ID No. 1865 | Seq. ID No. 1866 |
| 927 | Seq. ID No. 1867 | Seq. ID No. 1868 |
| 929b | Seq. ID No. 1871 | Seq. ID No. 1872 |
| 930 | Seq. ID No. 1873 | Seq. ID No. 1874 |
| 932 | Seq. ID No. 1877 | Seq. ID No. 1878 |
| 934a | Seq. ID No. 1879 | Seq. ID No. 1880 |
| 935 | Seq. ID No. 1887 | Seq. ID No. 1888 |
| 938 | Seq. ID No. 1889 | Seq. ID No. 1890 |
| 939c | Seq. ID No. 1895 | Seq. ID No. 1896 |
| 942 | Seq. ID No. 1897 | Seq. ID No. 1898 |
| 943 | Seq. ID No. 1899 | Seq. ID No. 1900 |
| 944a | Seq. ID No. 1901 | Seq. ID No. 1902 |
| 946 | Seq. ID No. 1905 | Seq. ID No. 1906 |
| 949b | Seq. ID No. 1909 | Seq. ID No. 1910 |
| 951a | Seq. ID No. 1917 | Seq. ID No. 1918 |
| 952a | Seq. ID No. 1921 | Seq. ID No. 1922 |
| 954 | Seq. ID No. 1925 | Seq. ID No. 1926 |
| 956 | Seq. ID No. 1931 | Seq. ID No. 1932 |
| 958a | Seq. ID No. 1933 | Seq. ID No. 1934 |
| 959 | Seq. ID No. 1937 | Seq. ID No. 1938 |
| 961a | Seq. ID No. 1939 | Seq. ID No. 1940 |
| 963 | Seq. ID No. 1945 | Seq. ID No. 1946 |
| 965a | Seq. ID No. 1947 | Seq. ID No. 1948 |
| 966a | Seq. ID No. 1951 | Seq. ID No. 1952 |
| 967a | Seq. ID No. 1955 | Seq. ID No. 1956 |
| 968 | Seq. ID No. 1959 | Seq. ID No. 1960 |
| 969 | Seq. ID No. 1961 | Seq. ID No. 1962 |
| 972b | Seq. ID No. 1965 | Seq. ID No. 1966 |
| 976 | Seq. ID No. 1967 | Seq. ID No. 1968 |
| 977a | Seq. ID No. 1969 | Seq. ID No. 1970 |
| 978a | Seq. ID No. 1973 | Seq. ID No. 1974 |
| 979 | Seq. ID No. 1977 | Seq. ID No. 1978 |
| 980 | Seq. ID No. 1979 | Seq. ID No. 1980 |
| 982 | Seq. ID No. 1983 | Seq. ID No. 1984 |
| 986 | Seq. ID No. 1989 | Seq. ID No. 1990 |
| 987 | Seq. ID No. 1991 | Seq. ID No. 1992 |
| 988c | Seq. ID No. 1997 | Seq. ID No. 1998 |
| 991 | Seq. ID No. 2001 | Seq. ID No. 2002 |
| 992 | Seq. ID No. 2003 | Seq. ID No. 2004 |
| 997 | Seq. ID No. 2011 | Seq. ID No. 2012 |
| 1000 | Seq. ID No. 2021 | Seq. ID No. 2022 |
| 1001 | Seq. ID No. 2023 | Seq. ID No. 2024 |
| 1004a | Seq. ID No. 2029 | Seq. ID No. 2030 |
| 1006a | Seq. ID No. 2033 | Seq. ID No. 2034 |
| 1007 | Seq. ID No. 2037 | Seq. ID No. 2038 |
| 1012 | Seq. ID No. 2039 | Seq. ID No. 2040 |
| 1013c | Seq. ID No. 2045 | Seq. ID No. 2046 |
| 1016c | Seq. ID No. 2047 | Seq. ID No. 2048 |
| 1017b | Seq. ID No. 2051 | Seq. ID No. 2052 |
| 1018a | Seq. ID No. 2057 | Seq. ID No. 2058 |
| 1021a | Seq. ID No. 2061 | Seq. ID No. 2062 |
| 1024 | Seq. ID No. 2067 | Seq. ID No. 2068 |
| 1025 | Seq. ID No. 2069 | Seq. ID No. 2070 |
| 1026 | Seq. ID No. 2071 | Seq. ID No. 2072 |
| 1029 | Seq. ID No. 2073 | Seq. ID No. 2074 |
| 1033b | Seq. ID No. 2077 | Seq. ID No. 2078 |
| 1036b | Seq. ID No. 2089 | Seq. ID No. 2090 |
| 1037 | Seq. ID No. 2093 | Seq. ID No. 2094 |
| 1039a | Seq. ID No. 2095 | Seq. ID No. 2096 |
| 1041 | Seq. ID No. 2099 | Seq. ID No. 2100 |
| 1042a | Seq. ID No. 2101 | Seq. ID No. 2102 |
| 1047a | Seq. ID No. 2113 | Seq. ID No. 2114 |
| 1049a | Seq. ID No. 2117 | Seq. ID No. 2118 |
| 1050b | Seq. ID No. 2123 | Seq. ID No. 2124 |
| 1057 | Seq. ID No. 2135 | Seq. ID No. 2136 |
| 1058 | Seq. ID No. 2137 | Seq. ID No. 2138 |
| 1060a | Seq. ID No. 2139 | Seq. ID No. 2140 |
| 1062 | Seq. ID No. 2143 | Seq. ID No. 2144 |
| 1064 | Seq. ID No. 2147 | Seq. ID No. 2148 |
| 1065c | Seq. ID No. 2153 | Seq. ID No. 2154 |
| 1067 | Seq. ID No. 2159 | Seq. ID No. 2160 |
| 1070a | Seq. ID No. 2163 | Seq. ID No. 2164 |
| 1076a | Seq. ID No. 2167 | Seq. ID No. 2168 |
| 1077c | Seq. ID No. 2175 | Seq. ID No. 2176 |
| 1079a | Seq. ID No. 2181 | Seq. ID No. 2182 |
| 1080a | Seq. ID No. 2185 | Seq. ID No. 2186 |
| 1081b | Seq. ID No. 2189 | Seq. ID No. 2190 |
| 1085 | Seq. ID No. 2193 | Seq. ID No. 2194 |
| 1086 | Seq. ID No. 2195 | Seq. ID No. 2196 |
| 1087a | Seq. ID No. 2197 | Seq. ID No. 2198 |
| 1094a | Seq. ID No. 2205 | Seq. ID No. 2206 |
| 1096c | Seq. ID No. 2217 | Seq. ID No. 2218 |
| 1097 | Seq. ID No. 2219 | Seq. ID No. 2220 |
| 1100 | Seq. ID No. 2223 | Seq. ID No. 2224 |
| 1101a | Seq. ID No. 2225 | Seq. ID No. 2226 |
| 1104 | Seq. ID No. 2229 | Seq. ID No. 2230 |
| 1108b | Seq. ID No. 2235 | Seq. ID No. 2236 |
| 1111b | Seq. ID No. 2239 | Seq. ID No. 2240 |
| 1112a | Seq. ID No. 2243 | Seq. ID No. 2244 |
| 1125 | Seq. ID No. 2245 | Seq. ID No. 2246 |
| 1126a | Seq. ID No. 2247 | Seq. ID No. 2248 |
| 1127 | Seq. ID No. 2253 | Seq. ID No. 2254 |
| 1128 | Seq. ID No. 2255 | Seq. ID No. 2256 |
| 1131 | Seq. ID No. 2257 | Seq. ID No. 2258 |
| 1132 | Seq. ID No. 2259 | Seq. ID No. 2260 |
| 1135a | Seq. ID No. 2261 | Seq. ID No. 2262 |
| 1136a | Seq. ID No. 2265 | Seq. ID No. 2266 |
| 1138 | Seq. ID No. 2269 | Seq. ID No. 2270 |
| 1140b | Seq. ID No. 2273 | Seq. ID No. 2274 |
| 1144b | Seq. ID No. 2285 | Seq. ID No. 2286 |
| 1146e | Seq. ID No. 2297 | Seq. ID No. 2298 |
| 1148 | Seq. ID No. 2299 | Seq. ID No. 2300 |
| 1149 | Seq. ID No. 2301 | Seq. ID No. 2302 |
| 1152f | Seq. ID No. 2313 | Seq. ID No. 2314 |
| 1155f | Seq. ID No. 2327 | Seq. ID No. 2328 |
| 1158a | Seq. ID No. 2331 | Seq. ID No. 2332 |
| 1160 | Seq. ID No. 2335 | Seq. ID No. 2336 |
| 1161a | Seq. ID No. 2337 | Seq. ID No. 2338 |
| 1162 | Seq. ID No. 2341 | Seq. ID No. 2342 |
| 1165 | Seq. ID No. 2343 | Seq. ID No. 2344 |
| 1166 | Seq. ID No. 2345 | Seq. ID No. 2346 |
| 1167b | Seq. ID No. 2349 | Seq. ID No. 2350 |
| 1168 | Seq. ID No. 2351 | Seq. ID No. 2352 |
| 1169c | Seq. ID No. 2357 | Seq. ID No. 2358 |
| 1170 | Seq. ID No. 2359 | Seq. ID No. 2360 |
| 1171a | Seq. ID No. 2361 | Seq. ID No. 2362 |
| 1172 | Seq. ID No. 2365 | Seq. ID No. 2366 |
| 1174 | Seq. ID No. 2369 | Seq. ID No. 2370 |
| 1177 | Seq. ID No. 2373 | Seq. ID No. 2374 |
| 1179a | Seq. ID No. 2377 | Seq. ID No. 2378 |
| 1180a | Seq. ID No. 2383 | Seq. ID No. 2384 |
| 1183a | Seq. ID No. 2397 | Seq. ID No. 2398 |
| 1184 | Seq. ID No. 2401 | Seq. ID No. 2402 |
| 1185a | Seq. ID No. 2403 | Seq. ID No. 2404 |
| 1186b | Seq. ID No. 2409 | Seq. ID No. 2410 |
| 1188a | Seq. ID No. 2411 | Seq. ID No. 2412 |
| 1191 | Seq. ID No. 2417 | Seq. ID No. 2418 |
| 1192b | Seq. ID No. 2421 | Seq. ID No. 2422 |
| 1194 | Seq. ID No. 2423 | Seq. ID No. 2424 |
| 1196a | Seq. ID No. 2425 | Seq. ID No. 2426 |
| 1197 | Seq. ID No. 2429 | Seq. ID No. 2430 |
| 1199b | Seq. ID No. 2435 | Seq. ID No. 2436 |
| 1200 | Seq. ID No. 2437 | Seq. ID No. 2438 |
| 1201 | Seq. ID No. 2439 | Seq. ID No. 2440 |
| 1202a | Seq. ID No. 2441 | Seq. ID No. 2442 |
| 1205 | Seq. ID No. 2445 | Seq. ID No. 2446 |
| 1207 | Seq. ID No. 2449 | Seq. ID No. 2450 |
| 1208 | Seq. ID No. 2451 | Seq. ID No. 2452 |
| 1209 | Seq. ID No. 2453 | Seq. ID No. 2454 |
| 1213a | Seq. ID No. 2461 | Seq. ID No. 2462 |
| 1214 | Seq. ID No. 2465 | Seq. ID No. 2466 |
| 1215a | Seq. ID No. 2467 | Seq. ID No. 2468 |
| 1217c | Seq. ID No. 2471 | Seq. ID No. 2472 |
| 1218a | Seq. ID No. 2473 | Seq. ID No. 2474 |
| 1219 | Seq. ID No. 2477 | Seq. ID No. 2478 |
| 1220 | Seq. ID No. 2479 | Seq. ID No. 2480 |
| 1221a | Seq. ID No. 2481 | Seq. ID No. 2482 |
| 1223 | Seq. ID No. 2485 | Seq. ID No. 2486 |
| 1224a | Seq. ID No. 2487 | Seq. ID No. 2488 |
| 1226 | Seq. ID No. 2495 | Seq. ID No. 2496 |
| 1230 | Seq. ID No. 2505 | Seq. ID No. 2506 |
| 1231 | Seq. ID No. 2507 | Seq. ID No. 2508 |
| 1233a | Seq. ID No. 2509 | Seq. ID No. 2510 |
| 1234c | Seq. ID No. 2519 | Seq. ID No. 2520 |
| 1237 | Seq. ID No. 2525 | Seq. ID No. 2526 |
| 1239 | Seq. ID No. 2527 | Seq. ID No. 2528 |
| 1240 | Seq. ID No. 2529 | Seq. ID No. 2530 |
| 1241c | Seq. ID No. 2535 | Seq. ID No. 2536 |
| 1243 | Seq. ID No. 2537 | Seq. ID No. 2538 |
| 1244 | Seq. ID No. 2539 | Seq. ID No. 2540 |
| 1245d | Seq. ID No. 2547 | Seq. ID No. 2548 |
| 1246a | Seq. ID No. 2551 | Seq. ID No. 2552 |
| 1250e | Seq. ID No. 2563 | Seq. ID No. 2564 |
| 1251a | Seq. ID No. 2565 | Seq. ID No. 2566 |
| 1253c | Seq. ID No. 2573 | Seq. ID No. 2574 |
| 1256 | Seq. ID No. 2577 | Seq. ID No. 2578 |
| 1257a | Seq. ID No. 2579 | Seq. ID No. 2580 |
| 1258b | Seq. ID No. 2585 | Seq. ID No. 2586 |
| 1263 | Seq. ID No. 2593 | Seq. ID No. 2594 |
| 1265 | Seq. ID No. 2595 | Seq. ID No. 2596 |
| 1266b | Seq. ID No. 2599 | Seq. ID No. 2600 |
| 1268b | Seq. ID No. 2607 | Seq. ID No. 2608 |
| 1269 | Seq. ID No. 2609 | Seq. ID No. 2610 |
| 1271 | Seq. ID No. 2613 | Seq. ID No. 2614 |
| 1272a | Seq. ID No. 2615 | Seq. ID No. 2616 |
| 1273 | Seq. ID No. 2619 | Seq. ID No. 2620 |
| 1275b | Seq. ID No. 2623 | Seq. ID No. 2624 |
| 1277 | Seq. ID No. 2625 | Seq. ID No. 2626 |
| 1279 | Seq. ID No. 2631 | Seq. ID No. 2632 |
| 1281 | Seq. ID No. 2633 | Seq. ID No. 2634 |
| 1283c | Seq. ID No. 2639 | Seq. ID No. 2640 |
| 1284 | Seq. ID No. 2641 | Seq. ID No. 2642 |
| 1285a | Seq. ID No. 2643 | Seq. ID No. 2644 |
| 1287a | Seq. ID No. 2647 | Seq. ID No. 2648 |
| 1288 | Seq. ID No. 2651 | Seq. ID No. 2652 |
| 1290 | Seq. ID No. 2655 | Seq. ID No. 2656 |
| 1293a | Seq. ID No. 2657 | Seq. ID No. 2658 |
| 1294a | Seq. ID No. 2661 | Seq. ID No. 2662 |
| 1297a | Seq. ID No. 2665 | Seq. ID No. 2666 |
| 1300 | Seq. ID No. 2669 | Seq. ID No. 2670 |
| 1302 | Seq. ID No. 2671 | Seq. ID No. 2672 |
| 1305a | Seq. ID No. 2673 | Seq. ID No. 2674 |
| 1308b | Seq. ID No. 2681 | Seq. ID No. 2682 |
| 1311 | Seq. ID No. 2689 | Seq. ID No. 2690 |
| 1315 | Seq. ID No. 2693 | Seq. ID No. 2694 |
| 1318 | Seq. ID No. 2699 | Seq. ID No. 2700 |
| 1319b | Seq. ID No. 2703 | Seq. ID No. 2704 |
| 1320c | Seq. ID No. 2709 | Seq. ID No. 2710 |
| 1321 | Seq. ID No. 2711 | Seq. ID No. 2712 |
| 1322a | Seq. ID No. 2713 | Seq. ID No. 2714 |
| 1324 | Seq. ID No. 2719 | Seq. ID No. 2720 |
| 1326a | Seq. ID No. 2721 | Seq. ID No. 2722 |
| 1333 | Seq. ID No. 2727 | Seq. ID No. 2728 |
| 1337 | Seq. ID No. 2737 | Seq. ID No. 2738 |
| 1340a | Seq. ID No. 2741 | Seq. ID No. 2742 |
| 1341a | Seq. ID No. 2745 | Seq. ID No. 2746 |
| 1344a | Seq. ID No. 2749 | Seq. ID No. 2750 |
| 1348 | Seq. ID No. 2753 | Seq. ID No. 2754 |
| 1349b | Seq. ID No. 2757 | Seq. ID No. 2758 |
| 1350a | Seq. ID No. 2761 | Seq. ID No. 2762 |
| 1353 | Seq. ID No. 2765 | Seq. ID No. 2766 |
| 1355 | Seq. ID No. 2767 | Seq. ID No. 2768 |
| 1358a | Seq. ID No. 2769 | Seq. ID No. 2770 |
| 1359 | Seq. ID No. 2773 | Seq. ID No. 2774 |
| 1361 | Seq. ID No. 2775 | Seq. ID No. 2776 |
| 1364a | Seq. ID No. 2777 | Seq. ID No. 2778 |
| 1366b | Seq. ID No. 2787 | Seq. ID No. 2788 |
| 1367 | Seq. ID No. 2789 | Seq. ID No. 2790 |
| 1368 | Seq. ID No. 2791 | Seq. ID No. 2792 |
| 1371 | Seq. ID No. 2793 | Seq. ID No. 2794 |
| 1373a | Seq. ID No. 2795 | Seq. ID No. 2796 |
| 1374 | Seq. ID No. 2799 | Seq. ID No. 2800 |
| 1377 | Seq. ID No. 2803 | Seq. ID No. 2804 |
| 1382 | Seq. ID No. 2815 | Seq. ID No. 2816 |
| 1383 | Seq. ID No. 2817 | Seq. ID No. 2818 |
| 1384a | Seq. ID No. 2819 | Seq. ID No. 2820 |
| 1386a | Seq. ID No. 2825 | Seq. ID No. 2826 |
| 1394 | Seq. ID No. 2843 | Seq. ID No. 2844 |
| 1396 | Seq. ID No. 2847 | Seq. ID No. 2848 |
| 1397 | Seq. ID No. 2849 | Seq. ID No. 2850 |
| 1398 | Seq. ID No. 2851 | Seq. ID No. 2852 |
| 1400 | Seq. ID No. 2857 | Seq. ID No. 2858 |
| 1401 | Seq. ID No. 2859 | Seq. ID No. 2860 |
| 1405 | Seq. ID No. 2863 | Seq. ID No. 2864 |
| 1408 | Seq. ID No. 2867 | Seq. ID No. 2868 |
| 1409 | Seq. ID No. 2869 | Seq. ID No. 2870 |
| 1410 | Seq. ID No. 2871 | Seq. ID No. 2872 |
| 1411c | Seq. ID No. 2877 | Seq. ID No. 2878 |
| 1412a | Seq. ID No. 2879 | Seq. ID No. 2880 |
| 1415 | Seq. ID No. 2889 | Seq. ID No. 2890 |
| 1416 | Seq. ID No. 2891 | Seq. ID No. 2892 |
| 1420a | Seq. ID No. 2897 | Seq. ID No. 2898 |
| 1422 | Seq. ID No. 2901 | Seq. ID No. 2902 |
| 1423 | Seq. ID No. 2903 | Seq. ID No. 2904 |
| 1426b | Seq. ID No. 2907 | Seq. ID No. 2908 |
| 1427a | Seq. ID No. 2909 | Seq. ID No. 2910 |
| 1428 | Seq. ID No. 2913 | Seq. ID No. 2914 |
| 1429a | Seq. ID No. 2915 | Seq. ID No. 2916 |
| 1431 | Seq. ID No. 2919 | Seq. ID No. 2920 |
| 1432 | Seq. ID No. 2921 | Seq. ID No. 2922 |
| 1433 | Seq. ID No. 2923 | Seq. ID No. 2924 |
| 1434a | Seq. ID No. 2925 | Seq. ID No. 2926 |
| 1437a | Seq. ID No. 2929 | Seq. ID No. 2930 |
| 1438 | Seq. ID No. 2935 | Seq. ID No. 2936 |
| 1439d | Seq. ID No. 2943 | Seq. ID No. 2944 |
| 1440c | Seq. ID No. 2951 | Seq. ID No. 2952 |
| 1442a | Seq. ID No. 2955 | Seq. ID No. 2956 |
| 1445c | Seq. ID No. 2963 | Seq. ID No. 2964 |
| 1446a | Seq. ID No. 2965 | Seq. ID No. 2966 |
| 1447b | Seq. ID No. 2971 | Seq. ID No. 2972 |
| 1448 | Seq. ID No. 2973 | Seq. ID No. 2974 |
| 1449a | Seq. ID No. 2975 | Seq. ID No. 2976 |
| 1451 | Seq. ID No. 2979 | Seq. ID No. 2980 |
| 1452a | Seq. ID No. 2981 | Seq. ID No. 2982 |
| 1453 | Seq. ID No. 2985 | Seq. ID No. 2986 |
| 1455a | Seq. ID No. 2989 | Seq. ID No. 2990 |
| 1456 | Seq. ID No. 2993 | Seq. ID No. 2994 |
| 1457c | Seq. ID No. 2999 | Seq. ID No. 3000 |
| 1459 | Seq. ID No. 3001 | Seq. ID No. 3002 |
| 1462a | Seq. ID No. 3003 | Seq. ID No. 3004 |
| 1466c | Seq. ID No. 3017 | Seq. ID No. 3018 |
| 1469c | Seq. ID No. 3027 | Seq. ID No. 3028 |
| 1470c | Seq. ID No. 3033 | Seq. ID No. 3034 |
| 1471 | Seq. ID No. 3035 | Seq. ID No. 3036 |
| 1472c | Seq. ID No. 3039 | Seq. ID No. 3040 |
| 1474 | Seq. ID No. 3041 | Seq. ID No. 3042 |
| 1475 | Seq. ID No. 3043 | Seq. ID No. 3044 |
| 1476 | Seq. ID No. 3045 | Seq. ID No. 3046 |
| 1477a | Seq. ID No. 3047 | Seq. ID No. 3048 |
| 1479c | Seq. ID No. 3055 | Seq. ID No. 3056 |
| 1482a | Seq. ID No. 3057 | Seq. ID No. 3058 |
| 1483 | Seq. ID No. 3061 | Seq. ID No. 3062 |
| 1484b | Seq. ID No. 3065 | Seq. ID No. 3066 |
| 1486 | Seq. ID No. 3069 | Seq. ID No. 3070 |
| 1488d | Seq. ID No. 3079 | Seq. ID No. 3080 |
| 1490 | Seq. ID No. 3089 | Seq. ID No. 3090 |
| 1494 | Seq. ID No. 3091 | Seq. ID No. 3092 |
| 1497 | Seq. ID No. 3093 | Seq. ID No. 3094 |
| 1498a | Seq. ID No. 3095 | Seq. ID No. 3096 |
| 1499 | Seq. ID No. 3103 | Seq. ID No. 3104 |
| 1502a | Seq. ID No. 3105 | Seq. ID No. 3106 |
| 1503a | Seq. ID No. 3109 | Seq. ID No. 3110 |
| 1506a | Seq. ID No. 3113 | Seq. ID No. 3114 |
| 1508 | Seq. ID No. 3125 | Seq. ID No. 3126 |
| 1510a | Seq. ID No. 3127 | Seq. ID No. 3128 |
| 1511 | Seq. ID No. 3135 | Seq. ID No. 3136 |
| 1512 | Seq. ID No. 3137 | Seq. ID No. 3138 |
| 1513b | Seq. ID No. 3141 | Seq. ID No. 3142 |
| 1516 | Seq. ID No. 3143 | Seq. ID No. 3144 |
| 1517b | Seq. ID No. 3147 | Seq. ID No. 3148 |
| 1520 | Seq. ID No. 3155 | Seq. ID No. 3156 |
| 1522 | Seq. ID No. 3157 | Seq. ID No. 3158 |
| 1523 | Seq. ID No. 3159 | Seq. ID No. 3160 |
| 1528 | Seq. ID No. 3161 | Seq. ID No. 3162 |
| 1530b | Seq. ID No. 3165 | Seq. ID No. 3166 |
| 1534a | Seq. ID No. 3169 | Seq. ID No. 3170 |
| 1536a | Seq. ID No. 3175 | Seq. ID No. 3176 |
| 1537 | Seq. ID No. 3179 | Seq. ID No. 3180 |
| 1543 | Seq. ID No. 3183 | Seq. ID No. 3184 |
| 1544 | Seq. ID No. 3185 | Seq. ID No. 3186 |
| 1547b | Seq. ID No. 3189 | Seq. ID No. 3190 |
| 1548 | Seq. ID No. 3191 | Seq. ID No. 3192 |
| 1553a | Seq. ID No. 3195 | Seq. ID No. 3196 |
| 1554a | Seq. ID No. 3199 | Seq. ID No. 3200 |
| 1555 | Seq. ID No. 3205 | Seq. ID No. 3206 |
| 1556c | Seq. ID No. 3211 | Seq. ID No. 3212 |
| 1557c | Seq. ID No. 3217 | Seq. ID No. 3218 |
| 1559b | Seq. ID No. 3221 | Seq. ID No. 3222 |
| 1564a | Seq. ID No. 3225 | Seq. ID No. 3226 |
| 1565 | Seq. ID No. 3229 | Seq. ID No. 3230 |
| 1567 | Seq. ID No. 3231 | Seq. ID No. 3232 |
| 1570 | Seq. ID No. 3237 | Seq. ID No. 3238 |
| 1571 | Seq. ID No. 3239 | Seq. ID No. 3240 |
| 1572d | Seq. ID No. 3247 | Seq. ID No. 3248 |
| 1573 | Seq. ID No. 3251 | Seq. ID No. 3252 |
| 1576 | Seq. ID No. 3253 | Seq. ID No. 3254 |
| 1577a | Seq. ID No. 3255 | Seq. ID No. 3256 |
| 1578 | Seq. ID No. 3259 | Seq. ID No. 3260 |
| 1580a | Seq. ID No. 3261 | Seq. ID No. 3262 |
| 1582a | Seq. ID No. 3267 | Seq. ID No. 3268 |
| 1584 | Seq. ID No. 3273 | Seq. ID No. 3274 |
| 1585b | Seq. ID No. 3277 | Seq. ID No. 3278 |
| 1587a | Seq. ID No. 3279 | Seq. ID No. 3280 |
| 1588 | Seq. ID No. 3285 | Seq. ID No. 3286 |
| 1590 | Seq. ID No. 3287 | Seq. ID No. 3288 |
| 1591 | Seq. ID No. 3289 | Seq. ID No. 3290 |
| 1592b | Seq. ID No. 3293 | Seq. ID No. 3294 |
| 1593 | Seq. ID No. 3295 | Seq. ID No. 3296 |
| 1594b | Seq. ID No. 3299 | Seq. ID No. 3300 |
| 1595a | Seq. ID No. 3301 | Seq. ID No. 3302 |
| 1598a | Seq. ID No. 3305 | Seq. ID No. 3306 |
| 1600a | Seq. ID No. 3309 | Seq. ID No. 3310 |
| 1602 | Seq. ID No. 3315 | Seq. ID No. 3316 |
| 1606b | Seq. ID No. 3319 | Seq. ID No. 3320 |
| 1607a | Seq. ID No. 3321 | Seq. ID No. 3322 |
| 1610a | Seq. ID No. 3327 | Seq. ID No. 3328 |
| 1616a | Seq. ID No. 3333 | Seq. ID No. 3334 |
| 1619 | Seq. ID No. 3337 | Seq. ID No. 3338 |
| 1623a | Seq. ID No. 3343 | Seq. ID No. 3344 |
| 1625b | Seq. ID No. 3353 | Seq. ID No. 3354 |
| 1626b | Seq. ID No. 3359 | Seq. ID No. 3360 |
| 1628a | Seq. ID No. 3361 | Seq. ID No. 3362 |
| 1632a | Seq. ID No. 3365 | Seq. ID No. 3366 |
| 1634a | Seq. ID No. 3373 | Seq. ID No. 3374 |
| 1642a | Seq. ID No. 3381 | Seq. ID No. 3382 |
| 1643a | Seq. ID No. 3385 | Seq. ID No. 3386 |
| 1646c | Seq. ID No. 3393 | Seq. ID No. 3394 |
| 1647 | Seq. ID No. 3401 | Seq. ID No. 3402 |
| 1649b | Seq. ID No. 3407 | Seq. ID No. 3408 |
| 1652a | Seq. ID No. 3411 | Seq. ID No. 3412 |
| 1654 | Seq. ID No. 3415 | Seq. ID No. 3416 |
| 1656b | Seq. ID No. 3417 | Seq. ID No. 3418 |
| 1659b | Seq. ID No. 3423 | Seq. ID No. 3424 |
| 1662a | Seq. ID No. 3429 | Seq. ID No. 3430 |
| 1665a | Seq. ID No. 3437 | Seq. ID No. 3438 |
| 1668 | Seq. ID No. 3439 | Seq. ID No. 3440 |
| 1670a | Seq. ID No. 3443 | Seq. ID No. 3444 |
| 1673f | Seq. ID No. 3459 | Seq. ID No. 3460 |
| 1674 | Seq. ID No. 346 | Seq. ID No. 3462 |
| 1675b | Seq. ID No. 3465 | Seq. ID No. 3466 |
| 1676 | Seq. ID No. 3469 | Seq. ID No. 3470 |
| 1677 | Seq. ID No. 3471 | Seq. ID No. 3472 |
| 1679 | Seq. ID No. 3473 | Seq. ID No. 3474 |
| 1680 | Seq. ID No. 3475 | Seq. ID No. 3476 |
| 1682a | Seq. ID No. 3477 | Seq. ID No. 3478 |
| 1683b | Seq. ID No. 3483 | Seq. ID No. 3484 |
| 1685 | Seq. ID No. 3487 | Seq. ID No. 3488 |
| 1688c | Seq. ID No. 3495 | Seq. ID No. 3496 |
| 1689a | Seq. ID No. 3497 | Seq. ID No. 3498 |
| 1690b | Seq. ID No. 3505 | Seq. ID No. 3506 |
| 1691 | Seq. ID No. 3507 | Seq. ID No. 3508 |
| 1692 | Seq. ID No. 3509 | Seq. ID No. 3510 |
| 1693 | Seq. ID No. 3511 | Seq. ID No. 3512 |
| 1694b | Seq. ID No. 3515 | Seq. ID No. 3516 |
| 1696 | Seq. ID No. 3521 | Seq. ID No. 3522 |
| 1697a | Seq. ID No. 3523 | Seq. ID No. 3524 |
| 1699b | Seq. ID No. 3529 | Seq. ID No. 3530 |
| 1701a | Seq. ID No. 3535 | Seq. ID No. 3536 |
| 1704a | Seq. ID No. 3541 | Seq. ID No. 3542 |
| 1707a | Seq. ID No. 3551 | Seq. ID No. 3552 |
| 1710a | Seq. ID No. 3559 | Seq. ID No. 3560 |
| 1712a | Seq. ID No. 3563 | Seq. ID No. 3564 |
| 1715a | Seq. ID No. 3567 | Seq. ID No. 3568 |
| 1722 | Seq. ID No. 3575 | Seq. ID No. 3576 |
| 1723a | Seq. ID No. 3577 | Seq. ID No. 3578 |
| 1727 | Seq. ID No. 3583 | Seq. ID No. 3584 |
| 1728 | Seq. ID No. 3585 | Seq. ID No. 3586 |
| 1733 | Seq. ID No. 3589 | Seq. ID No. 3590 |
| 1734 | Seq. ID No. 3591 | Seq. ID No. 3592 |
| 1737b | Seq. ID No. 3599 | Seq. ID No. 3600 |
| 1738a | Seq. ID No. 3601 | Seq. ID No. 3602 |
| 1739 | Seq. ID No. 3607 | Seq. ID No. 3608 |
| 1741 | Seq. ID No. 3609 | Seq. ID No. 3610 |
| 1743 | Seq. ID No. 3611 | Seq. ID No. 3612 |
| 1744b | Seq. ID No. 3615 | Seq. ID No. 3616 |
| 1747 | Seq. ID No. 3619 | Seq. ID No. 3620 |
| 1752b | Seq. ID No. 3629 | Seq. ID No. 3630 |
| 1753 | Seq. ID No. 3631 | Seq. ID No. 3632 |
| 1756a | Seq. ID No. 3633 | Seq. ID No. 3634 |
| 1757a | Seq. ID No. 3637 | Seq. ID No. 3638 |
| 1759 | Seq. ID No. 3641 | Seq. ID No. 3642 |
| 1760 | Seq. ID No. 3643 | Seq. ID No. 3644 |
| 1762 | Seq. ID No. 3645 | Seq. ID No. 3646 |
| 1764c | Seq. ID No. 3651 | Seq. ID No. 3652 |
| 1765 | Seq. ID No. 3653 | Seq. ID No. 3654 |
| 1766 | Seq. ID No. 3655 | Seq. ID No. 3656 |
| 1769a | Seq. ID No. 3661 | Seq. ID No. 3662 |
| 1771 | Seq. ID No. 3665 | Seq. ID No. 3666 |
| 1773 | Seq. ID No. 3667 | Seq. ID No. 3668 |
| 1774 | Seq. ID No. 3669 | Seq. ID No. 3670 |
| 1775c | Seq. ID No. 3675 | Seq. ID No. 3676 |
| 1780 | Seq. ID No. 3681 | Seq. ID No. 3682 |
| 1784b | Seq. ID No. 3689 | Seq. ID No. 3690 |
| 1788c | Seq. ID No. 3695 | Seq. ID No. 3696 |
| 1789a | Seq. ID No. 3697 | Seq. ID No. 3698 |
| 1792a | Seq. ID No. 3705 | Seq. ID No. 3706 |
| 1793a | Seq. ID No. 3711 | Seq. ID No. 3712 |
| 1797a | Seq. ID No. 3719 | Seq. ID No. 3720 |
| 1798b | Seq. ID No. 3725 | Seq. ID No. 3726 |
| 1800 | Seq. ID No. 3729 | Seq. ID No. 3730 |
| 1802a | Seq. ID No. 3731 | Seq. ID No. 3732 |
| 1803 | Seq. ID No. 3735 | Seq. ID No. 3736 |
| 1804b | Seq. ID No. 3739 | Seq. ID No. 3740 |
| 1805 | Seq. ID No. 3741 | Seq. ID No. 3742 |
| 1806a | Seq. ID No. 3743 | Seq. ID No. 3744 |
| 1807a | Seq. ID No. 3749 | Seq. ID No. 3750 |
| 1808d | Seq. ID No. 3759 | Seq. ID No. 3760 |
| 1809e | Seq. ID No. 3769 | Seq. ID No. 3770 |
| 1810a | Seq. ID No. 3773 | Seq. ID No. 3774 |
| 1811a | Seq. ID No. 3777 | Seq. ID No. 3778 |
| 1812a | Seq. ID No. 3781 | Seq. ID No. 3782 |
| 1815a | Seq. ID No. 3787 | Seq. ID No. 3788 |
| 1816b | Seq. ID No. 3795 | Seq. ID No. 3796 |
| 1819a | Seq. ID No. 3801 | Seq. ID No. 3802 |
| 1821 | Seq. ID No. 3805 | Seq. ID No. 3806 |
| 1823a | Seq. ID No. 3809 | Seq. ID No. 3810 |
| 1825 | Seq. ID No. 3813 | Seq. ID No. 3814 |
| 1828 | Seq. ID No. 3819 | Seq. ID No. 3820 |
| 1830a | Seq. ID No. 3821 | Seq. ID No. 3822 |
| 1833 | Seq. ID No. 3825 | Seq. ID No. 3826 |
| 1834 | Seq. ID No. 3827 | Seq. ID No. 3828 |
| 1835 | Seq. ID No. 3829 | Seq. ID No. 3830 |
| 1836a | Seq. ID No. 3831 | Seq. ID No. 3832 |
| 1837 | Seq. ID No. 3835 | Seq. ID No. 3836 |
| 1838c | Seq. ID No. 3841 | Seq. ID No. 3842 |
| 1839a | Seq. ID No. 3845 | Seq. ID No. 3846 |
| 1842d | Seq. ID No. 3855 | Seq. ID No. 3856 |
| 1843 | Seq. ID No. 3857 | Seq. ID No. 3858 |
| 1845c | Seq. ID No. 3863 | Seq. ID No. 3864 |
| 1848b | Seq. ID No. 3867 | Seq. ID No. 3868 |
| 1850a | Seq. ID No. 3869 | Seq. ID No. 3870 |
| 1853a | Seq. ID No. 3875 | Seq. ID No. 3876 |
| 1854b | Seq. ID No. 3881 | Seq. ID No. 3882 |
| 1855 | Seq. ID No. 3883 | Seq. ID No. 3884 |
| 1856 | Seq. ID No. 3885 | Seq. ID No. 3886 |
| 1857a | Seq. ID No. 3887 | Seq. ID No. 3888 |
| 1859 | Seq. ID No. 3897 | Seq. ID No. 3898 |
| 1861b | Seq. ID No. 3901 | Seq. ID No. 3902 |
| 1863a | Seq. ID No. 3905 | Seq. ID No. 3906 |
| 1864a | Seq. ID No. 3909 | Seq. ID No. 3910 |
| 1865b | Seq. ID No. 3913 | Seq. ID No. 3914 |
| 1868 | Seq. ID No. 3915 | Seq. ID No. 3916 |
| 1869 | Seq. ID No. 3917 | Seq. ID No. 3918 |
| 1870 | Seq. ID No. 3919 | Seq. ID No. 3920 |
| 1873a | Seq. ID No. 3921 | Seq. ID No. 3922 |
| 1875c | Seq. ID No. 3929 | Seq. ID No. 3930 |
| 1877a | Seq. ID No. 3931 | Seq. ID No. 3932 |
| 1878 | Seq. ID No. 3935 | Seq. ID No. 3936 |
| 1884 | Seq. ID No. 3941 | Seq. ID No. 3942 |
| 1886 | Seq. ID No. 3943 | Seq. ID No. 3944 |
| 1887 | Seq. ID No. 3945 | Seq. ID No. 3946 |
| 1888a | Seq. ID No. 3947 | Seq. ID No. 3948 |
| 1890 | Seq. ID No. 3951 | Seq. ID No. 3952 |
| 1891a | Seq. ID No. 3953 | Seq. ID No. 3954 |
| 1893 | Seq. ID No. 3957 | Seq. ID No. 3958 |
| 1897c | Seq. ID No. 3971 | Seq. ID No. 3972 |
| 1899 | Seq. ID No. 3977 | Seq. ID No. 3978 |
| 1902a | Seq. ID No. 3981 | Seq. ID No. 3982 |
| 1907 | Seq. ID No. 3985 | Seq. ID No. 3986 |
| 1912 | Seq. ID No. 3987 | Seq. ID No. 3988 |
| 1915a | Seq. ID No. 3989 | Seq. ID No. 3990 |
| 1916 | Seq. ID No. 3993 | Seq. ID No. 3994 |
| 1918c | Seq. ID No. 4001 | Seq. ID No. 4002 |
| 1919a | Seq. ID No. 4005 | Seq. ID No. 4006 |
| 1923a | Seq. ID No. 4011 | Seq. ID No. 4012 |
| 1925 | Seq. ID No. 4015 | Seq. ID No. 4016 |
| 1926 | Seq. ID No. 4017 | Seq. ID No. 4018 |
| 1927a | Seq. ID No. 4019 | Seq. ID No. 4020 |
| 1930 | Seq. ID No. 4023 | Seq. ID No. 4024 |
| 1931c | Seq. ID No. 4029 | Seq. ID No. 4030 |
| 1932 | Seq. ID No. 4031 | Seq. ID No. 4032 |
| 1933a | Seq. ID No. 4033 | Seq. ID No. 4034 |
| 1934 | Seq. ID No. 4037 | Seq. ID No. 4038 |
| 1936 | Seq. ID No. 4039 | Seq. ID No. 4040 |
| 1937e | Seq. ID No. 4049 | Seq. ID No. 4050 |
| 1939a | Seq. ID No. 4051 | Seq. ID No. 4052 |
| 1940 | Seq. ID No. 4057 | Seq. ID No. 4058 |
| 1941a | Seq. ID No. 4059 | Seq. ID No. 4060 |
| 1943a | Seq. ID No. 4065 | Seq. ID No. 4066 |
| 1944 | Seq. ID No. 4069 | Seq. ID No. 4070 |
| 1946c | Seq. ID No. 4075 | Seq. ID No. 4076 |
| 1950 | Seq. ID No. 4079 | Seq. ID No. 4080 |
| 1951c | Seq. ID No. 4085 | Seq. ID No. 4086 |
| 1953 | Seq. ID No. 4089 | Seq. ID No. 4090 |
| 1954c | Seq. ID No. 4095 | Seq. ID No. 4096 |
| 1956 | Seq. ID No. 4097 | Seq. ID No. 4098 |
| 1957 | Seq. ID No. 4099 | Seq. ID No. 4100 |
| 1958c | Seq. ID No. 4105 | Seq. ID No. 4106 |
| 1960c | Seq. ID No. 4111 | Seq. ID No. 4112 |
| 1962a | Seq. ID No. 4115 | Seq. ID No. 4116 |
| 1965b | Seq. ID No. 4121 | Seq. ID No. 4122 |
| 1966 | Seq. ID No. 4123 | Seq. ID No. 4124 |
| 1968 | Seq. ID No. 4127 | Seq. ID No. 4128 |
| 1969 | Seq. ID No. 4129 | Seq. ID No. 4130 |
| 1970a | Seq. ID No. 4131 | Seq. ID No. 4132 |
| 1973 | Seq. ID No. 4137 | Seq. ID No. 4138 |
| 1977a | Seq. ID No. 4141 | Seq. ID No. 4142 |
| 1979a | Seq. ID No. 4147 | Seq. ID No. 4148 |
| 1982 | Seq. ID No. 4157 | Seq. ID No. 4158 |
| 1985a | Seq. ID No. 4159 | Seq. ID No. 4160 |
| 1986 | Seq. ID No. 4163 | Seq. ID No. 4164 |
| 1987a | Seq. ID No. 4165 | Seq. ID No. 4166 |
| 1989a | Seq. ID No. 4169 | Seq. ID No. 4170 |
| 1990c | Seq. ID No. 4179 | Seq. ID No. 4180 |
| 1992a | Seq. ID No. 4183 | Seq. ID No. 4184 |
| 1993a | Seq. ID No. 4187 | Seq. ID No. 4188 |
| 1996 | Seq. ID No. 4193 | Seq. ID No. 4194 |
| 2000 | Seq. ID No. 4195 | Seq. ID No. 4196 |
| 2001 | Seq. ID No. 4197 | Seq. ID No. 4198 |
| 2003 | Seq. ID No. 4199 | Seq. ID No. 4200 |
| 2006 | Seq. ID No. 4201 | Seq. ID No. 4202 |
| 2010 | Seq. ID No. 4205 | Seq. ID No. 4206 |
| 2011a | Seq. ID No. 4207 | Seq. ID No. 4208 |
| 2013a | Seq. ID No. 4213 | Seq. ID No. 4214 |
| 2015 | Seq. ID No. 4219 | Seq. ID No. 4220 |
| 2016h | Seq. ID No. 4235 | Seq. ID No. 4236 |
| 2020 | Seq. ID No. 4237 | Seq. ID No. 4238 |
| 2021 | Seq. ID No. 4239 | Seq. ID No. 4240 |
| 2023b | Seq. ID No. 4243 | Seq. ID No. 4244 |
| 2024a | Seq. ID No. 4245 | Seq. ID No. 4246 |
| 2027 | Seq. ID No. 4255 | Seq. ID No. 4256 |
| 2030b | Seq. ID No. 4259 | Seq. ID No. 4260 |
| 2033 | Seq. ID No. 4267 | Seq. ID No. 4268 |
| 2036 | Seq. ID No. 4271 | Seq. ID No. 4272 |
| 2040a | Seq. ID No. 4277 | Seq. ID No. 4278 |
| 2042 | Seq. ID No. 4279 | Seq. ID No. 4280 |
| 2043b | Seq. ID No. 4281 | Seq. ID No. 4282 |
| 2044 | Seq. ID No. 4283 | Seq. ID No. 4284 |
| 2046a | Seq. ID No. 4289 | Seq. ID No. 4290 |
| 2047a | Seq. ID No. 4293 | Seq. ID No. 4294 |
| 2050 | Seq. ID No. 4299 | Seq. ID No. 4300 |
| 2053 | Seq. ID No. 4301 | Seq. ID No. 4302 |
| 2054 | Seq. ID No. 4303 | Seq. ID No. 4304 |
| 2055 | Seq. ID No. 4305 | Seq. ID No. 4306 |
| 2056 | Seq. ID No. 4307 | Seq. ID No. 4308 |
| 2057 | Seq. ID No. 4309 | Seq. ID No. 4310 |
| 2059b | Seq. ID No. 4313 | Seq. ID No. 4314 |
| 2060a | Seq. ID No. 4315 | Seq. ID No. 4316 |
| 2062c | Seq. ID No. 4323 | Seq. ID No. 4324 |
| 2064 | Seq. ID No. 4325 | Seq. ID No. 4326 |
| 2065a | Seq. ID No. 4327 | Seq. ID No. 4328 |
| 2066 | Seq. ID No. 4331 | Seq. ID No. 4332 |
| 2068 | Seq. ID No. 4333 | Seq. ID No. 4334 |
| 2069b | Seq. ID No. 4337 | Seq. ID No. 4338 |
| 2074a | Seq. ID No. 4341 | Seq. ID No. 4342 |
| 2075a | Seq. ID No. 4345 | Seq. ID No. 4346 |
| 2076a | Seq. ID No. 4349 | Seq. ID No. 4350 |
| 2078 | Seq. ID No. 4355 | Seq. ID No. 4356 |
| 2079a | Seq. ID No. 4357 | Seq. ID No. 4358 |
| 2081b | Seq. ID No. 4365 | Seq. ID No. 4366 |
| 2086a | Seq. ID No. 4367 | Seq. ID No. 4368 |
| 2087a | Seq. ID No. 4371 | Seq. ID No. 4372 |
| 2088 | Seq. ID No. 4375 | Seq. ID No. 4376 |
| 2091 | Seq. ID No. 4377 | Seq. ID No. 4378 |
| 2092 | Seq. ID No. 4379 | Seq. ID No. 4380 |
| 2094 | Seq. ID No. 4383 | Seq. ID No. 4384 |
| 2096b | Seq. ID No. 4387 | Seq. ID No. 4388 |
| 2097 | Seq. ID No. 4393 | Seq. ID No. 4394 |
| 2100 | Seq. ID No. 4397 | Seq. ID No. 4398 |
| 2104 | Seq. ID No. 4401 | Seq. ID No. 4402 |
| 2109a | Seq. ID No. 4409 | Seq. ID No. 4410 |
| 2110a | Seq. ID No. 4413 | Seq. ID No. 4414 |
| 2112b | Seq. ID No. 4421 | Seq. ID No. 4422 |
| 2116 | Seq. ID No. 4433 | Seq. ID No. 4434 |
| 2117 | Seq. ID No. 4435 | Seq. ID No. 4436 |
| 2124a | Seq. ID No. 4445 | Seq. ID No. 4446 |
| 2125a | Seq. ID No. 4449 | Seq. ID No. 4450 |
| 2128a | Seq. ID No. 4457 | Seq. ID No. 4458 |
| 2133 | Seq. ID No. 4461 | Seq. ID No. 4462 |
| 2138a | Seq. ID No. 4463 | Seq. ID No. 4464 |
| 2140b | Seq. ID No. 4471 | Seq. ID No. 4472 |
| 2142b | Seq. ID No. 4475 | Seq. ID No. 4476 |
| 2144a | Seq. ID No. 4477 | Seq. ID No. 4478 |
| 2146 | Seq. ID No. 4481 | Seq. ID No. 4482 |
| 2147c | Seq. ID No. 4487 | Seq. ID No. 4488 |
| 2149b | Seq. ID No. 4491 | Seq. ID No. 4492 |
| 2152a | Seq. ID No. 4499 | Seq. ID No. 4500 |
| 2153 | Seq. ID No. 4501 | Seq. ID No. 4502 |
| 2155a | Seq. ID No. 4503 | Seq. ID No. 4504 |
| 2156b | Seq. ID No. 4509 | Seq. ID No. 4510 |
| 2160a | Seq. ID No. 4517 | Seq. ID No. 4518 |
| 2164 | Seq. ID No. 4531 | Seq. ID No. 4532 |
| 2165 | Seq. ID No. 4533 | Seq. ID No. 4534 |
| 2166a | Seq. ID No. 4535 | Seq. ID No. 4536 |
| 2169 | Seq. ID No. 4539 | Seq. ID No. 4540 |
| 2171 | Seq. ID No. 4541 | Seq. ID No. 4542 |
| 2173a | Seq. ID No. 4545 | Seq. ID No. 4546 |
| 2174 | Seq. ID No. 4549 | Seq. ID No. 4550 |
| 2176c | Seq. ID No. 4557 | Seq. ID No. 4558 |
| 2179a | Seq. ID No. 4563 | Seq. ID No. 4564 |
| 2180 | Seq. ID No. 4567 | Seq. ID No. 4568 |
| 2181a | Seq. ID No. 4569 | Seq. ID No. 4570 |
| 2183 | Seq. ID No. 4575 | Seq. ID No. 4576 |
| 2185 | Seq. ID No. 4577 | Seq. ID No. 4578 |
| 2186a | Seq. ID No. 4579 | Seq. ID No. 4580 |
| 2189a | Seq. ID No. 4583 | Seq. ID No. 4584 |
| 2193 | Seq. ID No. 4591 | Seq. ID No. 4592 |
| 2194a | Seq. ID No. 4593 | Seq. ID No. 4594 |
| 2196 | Seq. ID No. 4597 | Seq. ID No. 4598 |
| 2197b | Seq. ID No. 4601 | Seq. ID No. 4602 |
| 2201a | Seq. ID No. 4605 | Seq. ID No. 4606 |
| 2203 | Seq. ID No. 4609 | Seq. ID No. 4610 |
| 2204 | Seq. ID No. 4611 | Seq. ID No. 4612 |
| 2206a | Seq. ID No. 4613 | Seq. ID No. 4614 |
| 2209 | Seq. ID No. 4623 | Seq. ID No. 4624 |
| 2212a | Seq. ID No. 4625 | Seq. ID No. 4626 |
| 2215a | Seq. ID No. 4629 | Seq. ID No. 4630 |
| 2216a | Seq. ID No. 4633 | Seq. ID No. 4634 |
| 2218 | Seq. ID No. 4637 | Seq. ID No. 4638 |
| 2219b | Seq. ID No. 4641 | Seq. ID No. 4642 |
| 2220a | Seq. ID No. 4645 | Seq. ID No. 4646 |
| 2221 | Seq. ID No. 4651 | Seq. ID No. 4652 |
| 2222 | Seq. ID No. 4653 | Seq. ID No. 4654 |
| 2223a | Seq. ID No. 4655 | Seq. ID No. 4656 |
| 2226 | Seq. ID No. 4661 | Seq. ID No. 4662 |
| 2227 | Seq. ID No. 4663 | Seq. ID No. 4664 |
| 2228c | Seq. ID No. 4669 | Seq. ID No. 4670 |
| 2231 | Seq. ID No. 4671 | Seq. ID No. 4672 |
| 2232 | Seq. ID No. 4673 | Seq. ID No. 4674 |
| 2234a | Seq. ID No. 4675 | Seq. ID No. 4676 |
| 2238 | Seq. ID No. 4685 | Seq. ID No. 4686 |
| 2240a | Seq. ID No. 4689 | Seq. ID No. 4690 |
| 2244 | Seq. ID No. 4697 | Seq. ID No. 4698 |
| 2248 | Seq. ID No. 4703 | Seq. ID No. 4704 |
| 2249 | Seq. ID No. 4705 | Seq. ID No. 4706 |
| 2253d | Seq. ID No. 4713 | Seq. ID No. 4714 |
| 2255b | Seq. ID No. 4717 | Seq. ID No. 4718 |
| 2256 | Seq. ID No. 4719 | Seq. ID No. 4720 |
| 2259a | Seq. ID No. 4723 | Seq. ID No. 4724 |
| 2260c | Seq. ID No. 4729 | Seq. ID No. 4730 |
| 2262 | Seq. ID No. 4731 | Seq. ID No. 4732 |
| 2263 | Seq. ID No. 4733 | Seq. ID No. 4734 |
| 2267 | Seq. ID No. 4739 | Seq. ID No. 4740 |
| 2269 | Seq. ID No. 4743 | Seq. ID No. 4744 |
| 2270 | Seq. ID No. 4745 | Seq. ID No. 4746 |
| 2272 | Seq. ID No. 4747 | Seq. ID No. 4748 |
| 2273 | Seq. ID No. 4749 | Seq. ID No. 4750 |
| 2274 | Seq. ID No. 4751 | Seq. ID No. 4752 |
| 2275a | Seq. ID No. 4753 | Seq. ID No. 4754 |
| 2276b | Seq. ID No. 4761 | Seq. ID No. 4762 |
| 2277a | Seq. ID No. 4767 | Seq. ID No. 4768 |
| 2278a | Seq. ID No. 4771 | Seq. ID No. 4772 |
| 2280 | Seq. ID No. 4779 | Seq. ID No. 4780 |
| 2284 | Seq. ID No. 4781 | Seq. ID No. 4782 |
| 2285 | Seq. ID No. 4783 | Seq. ID No. 4784 |
| 2286a | Seq. ID No. 4785 | Seq. ID No. 4786 |
| 2288 | Seq. ID No. 4789 | Seq. ID No. 4790 |
| 2290 | Seq. ID No. 4791 | Seq. ID No. 4792 |
| 2291a | Seq. ID No. 4793 | Seq. ID No. 4794 |
| 2292a | Seq. ID No. 4799 | Seq. ID No. 4800 |
| 2297 | Seq. ID No. 4807 | Seq. ID No. 4808 |
| 2298 | Seq. ID No. 4809 | Seq. ID No. 4810 |
| 2299 | Seq. ID No. 4811 | Seq. ID No. 4812 |
| 2300 | Seq. ID No. 4813 | Seq. ID No. 4814 |
| 2301 | Seq. ID No. 4815 | Seq. ID No. 4816 |
| 2304 | Seq. ID No. 4821 | Seq. ID No. 4822 |
| 2307 | Seq. ID No. 4825 | Seq. ID No. 4826 |
| 2308 | Seq. ID No. 4827 | Seq. ID No. 4828 |
| 2309a | Seq. ID No. 4829 | Seq. ID No. 4830 |
| 2311 | Seq. ID No. 4837 | Seq. ID No. 4838 |
| 2312a | Seq. ID No. 4839 | Seq. ID No. 4840 |
| 2314 | Seq. ID No. 4845 | Seq. ID No. 4846 |
| 2315 | Seq. ID No. 4847 | Seq. ID No. 4848 |
| 2316 | Seq. ID No. 4849 | Seq. ID No. 4850 |
| 2321b | Seq. ID No. 4857 | Seq. ID No. 4858 |
| 2322 | Seq. ID No. 4861 | Seq. ID No. 4862 |
| 2327 | Seq. ID No. 4865 | Seq. ID No. 4866 |
| 2328 | Seq. ID No. 4867 | Seq. ID No. 4868 |
| 2329d | Seq. ID No. 4875 | Seq. ID No. 4876 |
| 2331 | Seq. ID No. 4879 | Seq. ID No. 4880 |
| 2333 | Seq. ID No. 4881 | Seq. ID No. 4882 |
| 2335 | Seq. ID No. 4883 | Seq. ID No. 4884 |
| 2336 | Seq. ID No. 4885 | Seq. ID No. 4886 |
| 2337b | Seq. ID No. 4887 | Seq. ID No. 4888 |
| 2338c | Seq. ID No. 4893 | Seq. ID No. 4894 |
| 2342c | Seq. ID No. 4899 | Seq. ID No. 4900 |
| 2344 | Seq. ID No. 4901 | Seq. ID No. 4902 |
| 2345 | Seq. ID No. 4903 | Seq. ID No. 4904 |
| 2347 | Seq. ID No. 4905 | Seq. ID No. 4906 |
| 2348 | Seq. ID No. 4907 | Seq. ID No. 4908 |
| 2349a | Seq. ID No. 4909 | Seq. ID No. 4910 |
| 2353 | Seq. ID No. 4915 | Seq. ID No. 4916 |
| 2354 | Seq. ID No. 4917 | Seq. ID No. 4918 |
| 2357 | Seq. ID No. 4919 | Seq. ID No. 4920 |
| 2358 | Seq. ID No. 4921 | Seq. ID No. 4922 |
| 2361a | Seq. ID No. 4925 | Seq. ID No. 4926 |
| 2363a | Seq. ID No. 4929 | Seq. ID No. 4930 |
| 2365a | Seq. ID No. 4933 | Seq. ID No. 4934 |
| 2366a | Seq. ID No. 4937 | Seq. ID No. 4938 |
| 2367 | Seq. ID No. 4941 | Seq. ID No. 4942 |
| 2368 | Seq. ID No. 4943 | Seq. ID No. 4944 |
| 2369 | Seq. ID No. 4945 | Seq. ID No. 4946 |
| 2370 | Seq. ID No. 4947 | Seq. ID No. 4948 |
| 2373 | Seq. ID No. 4953 | Seq. ID No. 4954 |
| 2374a | Seq. ID No. 4955 | Seq. ID No. 4956 |
| 2376 | Seq. ID No. 4959 | Seq. ID No. 4960 |
| 2377a | Seq. ID No. 4961 | Seq. ID No. 4962 |
| 2378 | Seq. ID No. 4965 | Seq. ID No. 4966 |
| 2379a | Seq. ID No. 4967 | Seq. ID No. 4968 |
| 2380 | Seq. ID No. 4971 | Seq. ID No. 4972 |
| 2381 | Seq. ID No. 4973 | Seq. ID No. 4974 |
| 2384a | Seq. ID No. 4977 | Seq. ID No. 4978 |
| 2387 | Seq. ID No. 4985 | Seq. ID No. 4986 |
| 2391 | Seq. ID No. 4989 | Seq. ID No. 4990 |
| 2392 | Seq. ID No. 4991 | Seq. ID No. 4992 |
| 2395 | Seq. ID No. 4995 | Seq. ID No. 4996 |
| 2396 | Seq. ID No. 4997 | Seq. ID No. 4998 |
| 2399b | Seq. ID No. 5005 | Seq. ID No. 5006 |
| 2401 | Seq. ID No. 5009 | Seq. ID No. 5010 |
| 2403a | Seq. ID No. 5015 | Seq. ID No. 5016 |
| 2404a | Seq. ID No. 5019 | Seq. ID No. 5020 |
| 2409a | Seq. ID No. 5025 | Seq. ID No. 5026 |
| 2410 | Seq. ID No. 5029 | Seq. ID No. 5030 |
| 2415a | Seq. ID No. 5037 | Seq. ID No. 5038 |
| 2417a | Seq. ID No. 5045 | Seq. ID No. 5046 |
| 2418a | Seq. ID No. 5049 | Seq. ID No. 5050 |
| 2420 | Seq. ID No. 5053 | Seq. ID No. 5054 |
| 2421 | Seq. ID No. 5055 | Seq. ID No. 5056 |
| 2422c | Seq. ID No. 5059 | Seq. ID No. 5060 |
| 2423a | Seq. ID No. 5061 | Seq. ID No. 5062 |
| 2424a | Seq. ID No. 5065 | Seq. ID No. 5066 |
| 2426 | Seq. ID No. 5071 | Seq. ID No. 5072 |
| 2428a | Seq. ID No. 5073 | Seq. ID No. 5074 |
| 2429 | Seq. ID No. 5079 | Seq. ID No. 5080 |
| 2432 | Seq. ID No. 5081 | Seq. ID No. 5082 |
| 2433 | Seq. ID No. 5083 | Seq. ID No. 5084 |
| 2440 | Seq. ID No. 5087 | Seq. ID No. 5088 |
| 2442a | Seq. ID No. 5089 | Seq. ID No. 5090 |
| 2443 | Seq. ID No. 5093 | Seq. ID No. 5094 |
| 2444a | Seq. ID No. 5095 | Seq. ID No. 5096 |
| 2445 | Seq. ID No. 5103 | Seq. ID No. 5104 |
| 2448b | Seq. ID No. 5105 | Seq. ID No. 5106 |
| 2449a | Seq. ID No. 5107 | Seq. ID No. 5108 |
| 2451 | Seq. ID No. 5111 | Seq. ID No. 5112 |
| 2452 | Seq. ID No. 5113 | Seq. ID No. 5114 |
| 2453 | Seq. ID No. 5115 | Seq. ID No. 5116 |
| 2455 | Seq. ID No. 5117 | Seq. ID No. 5118 |
| 2456a | Seq. ID No. 5119 | Seq. ID No. 5120 |
| 2458g | Seq. ID No. 5141 | Seq. ID No. 5142 |
| 2461a | Seq. ID No. 5143 | Seq. ID No. 5144 |
| 2462a | Seq. ID No. 5147 | Seq. ID No. 5148 |
| 2464 | Seq. ID No. 5153 | Seq. ID No. 5154 |
| 2465a | Seq. ID No. 5155 | Seq. ID No. 5156 |
| 2469a | Seq. ID No. 5163 | Seq. ID No. 5164 |
| 2471 | Seq. ID No. 5169 | Seq. ID No. 5170 |
| 2473 | Seq. ID No. 5171 | Seq. ID No. 5172 |
| 2474a | Seq. ID No. 5173 | Seq. ID No. 5174 |
| 2475a | Seq. ID No. 5179 | Seq. ID No. 5180 |
| 2477 | Seq. ID No. 5185 | Seq. ID No. 5186 |
| 2480 | Seq. ID No. 5187 | Seq. ID No. 5188 |
| 2482 | Seq. ID No. 5193 | Seq. ID No. 5194 |
| 2483 | Seq. ID No. 5195 | Seq. ID No. 5196 |
| 2485 | Seq. ID No. 5201 | Seq. ID No. 5202 |
| 2489 | Seq. ID No. 5203 | Seq. ID No. 5204 |
| 2490 | Seq. ID No. 5205 | Seq. ID No. 5206 |
| 2492b | Seq. ID No. 5209 | Seq. ID No. 5210 |
| 2494 | Seq. ID No. 5211 | Seq. ID No. 5212 |
| 2495a | Seq. ID No. 5213 | Seq. ID No. 5214 |
| 2497a | Seq. ID No. 5217 | Seq. ID No. 5218 |
| 2500b | Seq. ID No. 5225 | Seq. ID No. 5226 |
| 2501b | Seq. ID No. 5231 | Seq. ID No. 5232 |
| 2504a | Seq. ID No. 5233 | Seq. ID No. 5234 |
| 2505 | Seq. ID No. 5237 | Seq. ID No. 5238 |
| 2506 | Seq. ID No. 5239 | Seq. ID No. 5240 |
| 2510 | Seq. ID No. 5245 | Seq. ID No. 5246 |
| 2512c | Seq. ID No. 5247 | Seq. ID No. 5248 |
| 2513a | Seq. ID No. 5249 | Seq. ID No. 5250 |
| 2514 | Seq. ID No. 5255 | Seq. ID No. 5256 |
| 2515 | Seq. ID No. 5257 | Seq. ID No. 5258 |
| 2516a | Seq. ID No. 5259 | Seq. ID No. 5260 |
| 2517a | Seq. ID No. 5263 | Seq. ID No. 5264 |
| 2519a | Seq. ID No. 5269 | Seq. ID No. 5270 |
| 2520a | Seq. ID No. 5273 | Seq. ID No. 5274 |
| 2523 | Seq. ID No. 5283 | Seq. ID No. 5284 |
| 2527a | Seq. ID No. 5289 | Seq. ID No. 5290 |
| 2528a | Seq. ID No. 5293 | Seq. ID No. 5294 |
| 2535a | Seq. ID No. 5307 | Seq. ID No. 5308 |
| 2538 | Seq. ID No. 5311 | Seq. ID No. 5312 |
| 2541 | Seq. ID No. 5313 | Seq. ID No. 5314 |
| 2542a | Seq. ID No. 5315 | Seq. ID No. 5316 |
| 2544 | Seq. ID No. 5323 | Seq. ID No. 5324 |
| 2545 | Seq. ID No. 5325 | Seq. ID No. 5326 |
| 2548b | Seq. ID No. 5335 | Seq. ID No. 5336 |
| 2549 | Seq. ID No. 5337 | Seq. ID No. 5338 |
| 2550b | Seq. ID No. 5339 | Seq. ID No. 5340 |
| 2552 | Seq. ID No. 5345 | Seq. ID No. 5346 |
| 2553 | Seq. ID No. 5347 | Seq. ID No. 5348 |
| 2554a | Seq. ID No. 5349 | Seq. ID No. 5350 |
| 2555a | Seq. ID No. 5353 | Seq. ID No. 5354 |
| 2558a | Seq. ID No. 5361 | Seq. ID No. 5362 |
| 2559 | Seq. ID No. 5365 | Seq. ID No. 5366 |
| 2561 | Seq. ID No. 5367 | Seq. ID No. 5368 |
| 2562a | Seq. ID No. 5369 | Seq. ID No. 5370 |
| 2563 | Seq. ID No. 5373 | Seq. ID No. 5374 |
| 2564a | Seq. ID No. 5375 | Seq. ID No. 5376 |
| 2567b | Seq. ID No. 5387 | Seq. ID No. 5388 |
| 2568 | Seq. ID No. 5389 | Seq. ID No. 5390 |
| 2572 | Seq. ID No. 5391 | Seq. ID No. 5392 |
| 2573 | Seq. ID No. 5393 | Seq. ID No. 5394 |
| 2575 | Seq. ID No. 5395 | Seq. ID No. 5396 |
| 2576 | Seq. ID No. 5397 | Seq. ID No. 5398 |
| 2578 | Seq. ID No. 5399 | Seq. ID No. 5400 |
| 2579b | Seq. ID No. 5401 | Seq. ID No. 5402 |
| 2581 | Seq. ID No. 5403 | Seq. ID No. 5404 |
| 2582a | Seq. ID No. 5405 | Seq. ID No. 5406 |
| 2585 | Seq. ID No. 5411 | Seq. ID No. 5412 |
| 2587b | Seq. ID No. 5415 | Seq. ID No. 5416 |
| 2588 | Seq. ID No. 5417 | Seq. ID No. 5418 |
| 2589 | Seq. ID No. 5419 | Seq. ID No. 5420 |
| 2591 | Seq. ID No. 5421 | Seq. ID No. 5422 |
| 2592 | Seq. ID No. 5423 | Seq. ID No. 5424 |
| 2594a | Seq. ID No. 5429 | Seq. ID No. 5430 |
| 2596 | Seq. ID No. 5433 | Seq. ID No. 5434 |
| 2598 | Seq. ID No. 5441 | Seq. ID No. 5442 |
| 2602 | Seq. ID No. 5443 | Seq. ID No. 5444 |
| 2605a | Seq. ID No. 5445 | Seq. ID No. 5446 |
| 2606 | Seq. ID No. 5449 | Seq. ID No. 5450 |
| 2608 | Seq. ID No. 5451 | Seq. ID No. 5452 |
| 2610 | Seq. ID No. 5453 | Seq. ID No. 5454 |
| 2611 | Seq. ID No. 5455 | Seq. ID No. 5456 |
| 2613 | Seq. ID No. 5457 | Seq. ID No. 5458 |
| 2616f | Seq. ID No. 5469 | Seq. ID No. 5470 |
| 2617a | Seq. ID No. 5475 | Seq. ID No. 5476 |
| 2619 | Seq. ID No. 5483 | Seq. ID No. 5484 |
| 2620 | Seq. ID No. 5485 | Seq. ID No. 5486 |
| 2623a | Seq. ID No. 5489 | Seq. ID No. 5490 |
| 2624a | Seq. ID No. 5493 | Seq. ID No. 5494 |
| 2625b | Seq. ID No. 5499 | Seq. ID No. 5500 |
| 2626a | Seq. ID No. 5501 | Seq. ID No. 5502 |
| 2627a | Seq. ID No. 5505 | Seq. ID No. 5506 |
| 2628 | Seq. ID No. 5509 | Seq. ID No. 5510 |
| 2629a | Seq. ID No. 5511 | Seq. ID No. 5512 |
| 2633c | Seq. ID No. 5523 | Seq. ID No. 5524 |
| 2637a | Seq. ID No. 5529 | Seq. ID No. 5530 |
| 2643a | Seq. ID No. 5535 | Seq. ID No. 5536 |
| 2645 | Seq. ID No. 5539 | Seq. ID No. 5540 |
| 2647 | Seq. ID No. 5541 | Seq. ID No. 5542 |
| 2649 | Seq. ID No. 5547 | Seq. ID No. 5548 |
| 2652a | Seq. ID No. 5551 | Seq. ID No. 5552 |
| 2655a | Seq. ID No. 5555 | Seq. ID No. 5556 |
| 2656 | Seq. ID No. 5559 | Seq. ID No. 5560 |
| 2658a | Seq. ID No. 5561 | Seq. ID No. 5562 |
| 2659a | Seq. ID No. 5565 | Seq. ID No. 5566 |
| 2662 | Seq. ID No. 5571 | Seq. ID No. 5572 |
| 2664a | Seq. ID No. 5573 | Seq. ID No. 5574 |
| 2666a | Seq. ID No. 5579 | Seq. ID No. 5580 |
| 2667a | Seq. ID No. 5583 | Seq. ID No. 5584 |
| 2669b | Seq. ID No. 5589 | Seq. ID No. 5590 |
| 2674b | Seq. ID No. 5599 | Seq. ID No. 5600 |
| 2676 | Seq. ID No. 5601 | Seq. ID No. 5602 |
| 2677a | Seq. ID No. 5603 | Seq. ID No. 5604 |
| 2679c | Seq. ID No. 5613 | Seq. ID No. 5614 |
| 2680c | Seq. ID No. 5621 | Seq. ID No. 5622 |
| 2684a | Seq. ID No. 5627 | Seq. ID No. 5628 |
| 2686 | Seq. ID No. 5631 | Seq. ID No. 5632 |
| 2688b | Seq. ID No. 5635 | Seq. ID No. 5636 |
| 2690b | Seq. ID No. 5645 | Seq. ID No. 5646 |
| 2693 | Seq. ID No. 5651 | Seq. ID No. 5652 |
| 2694a | Seq. ID No. 5653 | Seq. ID No. 5654 |
| 2695 | Seq. ID No. 5657 | Seq. ID No. 5658 |
| 2696b | Seq. ID No. 5661 | Seq. ID No. 5662 |
| 2705 | Seq. ID No. 5663 | Seq. ID No. 5664 |
| 2709 | Seq. ID No. 5665 | Seq. ID No. 5666 |
| 2712 | Seq. ID No. 5669 | Seq. ID No. 5670 |
| 2713 | Seq. ID No. 5671 | Seq. ID No. 5672 |
| 2714 | Seq. ID No. 5673 | Seq. ID No. 5674 |
| 2716a | Seq. ID No. 5675 | Seq. ID No. 5676 |
| 2718a | Seq. ID No. 5681 | Seq. ID No. 5682 |
| 2721b | Seq. ID No. 5693 | Seq. ID No. 5694 |
| 2722a | Seq. ID No. 5695 | Seq. ID No. 5696 |
| 2723 | Seq. ID No. 5699 | Seq. ID No. 5700 |
| 2724 | Seq. ID No. 5701 | Seq. ID No. 5702 |
| 2726 | Seq. ID No. 5707 | Seq. ID No. 5708 |
| 2731a | Seq. ID No. 5713 | Seq. ID No. 5714 |
| 2732 | Seq. ID No. 5717 | Seq. ID No. 5718 |
| 2734c | Seq. ID No. 5723 | Seq. ID No. 5724 |
| 2735a | Seq. ID No. 5725 | Seq. ID No. 5726 |
| 2738b | Seq. ID No. 5731 | Seq. ID No. 5732 |
| 2742a | Seq. ID No. 5737 | Seq. ID No. 5738 |
| 2745a | Seq. ID No. 5747 | Seq. ID No. 5748 |
| 2746 | Seq. ID No. 5751 | Seq. ID No. 5752 |
| 2747b | Seq. ID No. 5755 | Seq. ID No. 5756 |
| 2748b | Seq. ID No. 5759 | Seq. ID No. 5760 |
| 2749e | Seq. ID No. 5771 | Seq. ID No. 5772 |
| 2750a | Seq. ID No. 5773 | Seq. ID No. 5774 |
| 2752 | Seq. ID No. 5777 | Seq. ID No. 5778 |
| 2753 | Seq. ID No. 5779 | Seq. ID No. 5780 |
| 2757a | Seq. ID No. 5781 | Seq. ID No. 5782 |
| 2758a | Seq. ID No. 5785 | Seq. ID No. 5786 |
| 2759a | Seq. ID No. 5793 | Seq. ID No. 5794 |
| 2760b | Seq. ID No. 5799 | Seq. ID No. 5800 |
| 2763a | Seq. ID No. 5805 | Seq. ID No. 5806 |
| 2764 | Seq. ID No. 5811 | Seq. ID No. 5812 |
| 2765c | Seq. ID No. 5815 | Seq. ID No. 5816 |
| 2766a | Seq. ID No. 5817 | Seq. ID No. 5818 |
| 2768b | Seq. ID No. 5823 | Seq. ID No. 5824 |
| 2771a | Seq. ID No. 5831 | Seq. ID No. 5832 |
| 2773 | Seq. ID No. 5835 | Seq. ID No. 5836 |
| 2774b | Seq. ID No. 5839 | Seq. ID No. 5840 |
| 2776a | Seq. ID No. 5843 | Seq. ID No. 5844 |
| 2777 | Seq. ID No. 5847 | Seq. ID No. 5848 |
| 2778 | Seq. ID No. 5849 | Seq. ID No. 5850 |
| 2779 | Seq. ID No. 5851 | Seq. ID No. 5852 |
| 2780 | Seq. ID No. 5853 | Seq. ID No. 5854 |
| 2784 | Seq. ID No. 5855 | Seq. ID No. 5856 |
| 2785a | Seq. ID No. 5857 | Seq. ID No. 5858 |
| 2786a | Seq. ID No. 5861 | Seq. ID No. 5862 |
| 2787a | Seq. ID No. 5867 | Seq. ID No. 5868 |
| 2789 | Seq. ID No. 5875 | Seq. ID No. 5876 |
| 2790 | Seq. ID No. 5877 | Seq. ID No. 5878 |
| 2793 | Seq. ID No. 5881 | Seq. ID No. 5882 |
| 2799a | Seq. ID No. 5889 | Seq. ID No. 5890 |
| 2801b | Seq. ID No. 5895 | Seq. ID No. 5896 |
| 2804 | Seq. ID No. 5897 | Seq. ID No. 5898 |
| 2805b | Seq. ID No. 5901 | Seq. ID No. 5902 |
| 2809 | Seq. ID No. 5903 | Seq. ID No. 5904 |
| 2810b | Seq. ID No. 5907 | Seq. ID No. 5908 |
| 2812 | Seq. ID No. 5913 | Seq. ID No. 5914 |
| 2814 | Seq. ID No. 5915 | Seq. ID No. 5916 |
| 2815 | Seq. ID No. 5917 | Seq. ID No. 5918 |
| 2816 | Seq. ID No. 5919 | Seq. ID No. 5920 |
| 2818c | Seq. ID No. 5925 | Seq. ID No. 5926 |
| 2819 | Seq. ID No. 5931 | Seq. ID No. 5932 |
| 2820 | Seq. ID No. 5933 | Seq. ID No. 5934 |
| 2821a | Seq. ID No. 5935 | Seq. ID No. 5936 |
| 2822b | Seq. ID No. 5941 | Seq. ID No. 5942 |
| 2823 | Seq. ID No. 5943 | Seq. ID No. 5944 |
| 2824a | Seq. ID No. 5945 | Seq. ID No. 5946 |
| 2829a | Seq. ID No. 5949 | Seq. ID No. 5950 |
| 2832 | Seq. ID No. 5955 | Seq. ID No. 5956 |
| 2833 | Seq. ID No. 5957 | Seq. ID No. 5958 |
| 2834a | Seq. ID No. 5959 | Seq. ID No. 5960 |
| 2842a | Seq. ID No. 5967 | Seq. ID No. 5968 |
| 2843a | Seq. ID No. 5971 | Seq. ID No. 5972 |
| 2845 | Seq. ID No. 5975 | Seq. ID No. 5976 |
| 2846a | Seq. ID No. 5977 | Seq. ID No. 5978 |
| 2848 | Seq. ID No. 5981 | Seq. ID No. 5982 |
| 2850 | Seq. ID No. 5983 | Seq. ID No. 5984 |
| 2851 | Seq. ID No. 5985 | Seq. ID No. 5986 |
| 2853 | Seq. ID No. 5989 | Seq. ID No. 5990 |
| 2854a | Seq. ID No. 5991 | Seq. ID No. 5992 |
| 2855 | Seq. ID No. 5995 | Seq. ID No. 5996 |
| 2856a | Seq. ID No. 5997 | Seq. ID No. 5998 |
| 2857 | Seq. ID No. 6003 | Seq. ID No. 6004 |
| 2859 | Seq. ID No. 6005 | Seq. ID No. 6006 |
| 2860a | Seq. ID No. 6007 | Seq. ID No. 6008 |
| 2863 | Seq. ID No. 6015 | Seq. ID No. 6016 |
| 2864 | Seq. ID No. 6017 | Seq. ID No. 6018 |
| 2868a | Seq. ID No. 6019 | Seq. ID No. 6020 |
| 2869a | Seq. ID No. 6023 | Seq. ID No. 6024 |
| 2871 | Seq. ID No. 6029 | Seq. ID No. 6030 |
| 2875 | Seq. ID No. 6033 | Seq. ID No. 6034 |
| 2877 | Seq. ID No. 6035 | Seq. ID No. 6036 |
| 2879a | Seq. ID No. 6037 | Seq. ID No. 6038 |
| 2881 | Seq. ID No. 6043 | Seq. ID No. 6044 |
| 2883b | Seq. ID No. 6049 | Seq. ID No. 6050 |
| 2886a | Seq. ID No. 6055 | Seq. ID No. 6056 |
| 2890 | Seq. ID No. 6065 | Seq. ID No. 6066 |
| 2891b | Seq. ID No. 6069 | Seq. ID No. 6070 |
| 2894a | Seq. ID No. 6075 | Seq. ID No. 6076 |
| 2895 | Seq. ID No. 6081 | Seq. ID No. 6082 |
| 2897a | Seq. ID No. 6083 | Seq. ID No. 6084 |
| 2900a | Seq. ID No. 6087 | Seq. ID No. 6088 |
| 2902 | Seq. ID No. 6095 | Seq. ID No. 6096 |
| 2904 | Seq. ID No. 6097 | Seq. ID No. 6098 |
| 2905a | Seq. ID No. 6099 | Seq. ID No. 6100 |
| 2906 | Seq. ID No. 6103 | Seq. ID No. 6104 |
| 2909a | Seq. ID No. 6105 | Seq. ID No. 6106 |
| 2912 | Seq. ID No. 6113 | Seq. ID No. 6114 |
| 2913a | Seq. ID No. 6115 | Seq. ID No. 6116 |
| 2915a | Seq. ID No. 6119 | Seq. ID No. 6120 |
| 2916 | Seq. ID No. 6125 | Seq. ID No. 6126 |
| 2917 | Seq. ID No. 6127 | Seq. ID No. 6128 |
| 2918 | Seq. ID No. 6129 | Seq. ID No. 6130 |
| 2920 | Seq. ID No. 6131 | Seq. ID No. 6132 |
| 2921 | Seq. ID No. 6133 | Seq. ID No. 6134 |
| 2923c | Seq. ID No. 6141 | Seq. ID No. 6142 |
| 2925a | Seq. ID No. 6143 | Seq. ID No. 6144 |
| 2926a | Seq. ID No. 6147 | Seq. ID No. 6148 |
| 2928a | Seq. ID No. 6153 | Seq. ID No. 6154 |
| 2931c | Seq. ID No. 6165 | Seq. ID No. 6166 |
| 2933b | Seq. ID No. 6169 | Seq. ID No. 6170 |
| 2934 | Seq. ID No. 6171 | Seq. ID No. 6172 |
| 2935a | Seq. ID No. 6173 | Seq. ID No. 6174 |
| 2936 | Seq. ID No. 6177 | Seq. ID No. 6178 |
| 2939a | Seq. ID No. 6179 | Seq. ID No. 6180 |
| 2941c | Seq. ID No. 6187 | Seq. ID No. 6188 |
| 2943c | Seq. ID No. 6193 | Seq. ID No. 6194 |
| 2947 | Seq. ID No. 6201 | Seq. ID No. 6202 |
| 2948c | Seq. ID No. 6207 | Seq. ID No. 6208 |
| 2950a | Seq. ID No. 6211 | Seq. ID No. 6212 |
| 2951 | Seq. ID No. 6215 | Seq. ID No. 6216 |
| 2954 | Seq. ID No. 6219 | Seq. ID No. 6220 |
| 2955f | Seq. ID No. 6231 | Seq. ID No. 6232 |
| 2957 | Seq. ID No. 6235 | Seq. ID No. 6236 |
| 2959 | Seq. ID No. 6243 | Seq. ID No. 6244 |
| 2962a | Seq. ID No. 6247 | Seq. ID No. 6248 |
| 2963 | Seq. ID No. 6251 | Seq. ID No. 6252 |
| 2965b | Seq. ID No. 6255 | Seq. ID No. 6256 |
| 2967 | Seq. ID No. 6261 | Seq. ID No. 6262 |
| 2969 | Seq. ID No. 6263 | Seq. ID No. 6264 |
| 2971a | Seq. ID No. 6269 | Seq. ID No. 6270 |
| 2974b | Seq. ID No. 6275 | Seq. ID No. 6276 |
| 2975 | Seq. ID No. 6277 | Seq. ID No. 6278 |
| 2977a | Seq. ID No. 6279 | Seq. ID No. 6280 |
| 2979 | Seq. ID No. 6287 | Seq. ID No. 6288 |
| 2980b | Seq. ID No. 6291 | Seq. ID No. 6292 |
| 2984 | Seq. ID No. 6299 | Seq. ID No. 6300 |
| 2986c | Seq. ID No. 6305 | Seq. ID No. 6306 |
| 2988a | Seq. ID No. 6307 | Seq. ID No. 6308 |
| 2989 | Seq. ID No. 6313 | Seq. ID No. 6314 |
| 2990 | Seq. ID No. 6315 | Seq. ID No. 6316 |
| 2991c | Seq. ID No. 6321 | Seq. ID No. 6322 |
| 2992 | Seq. ID No. 6323 | Seq. ID No. 6324 |
| 2993a | Seq. ID No. 6325 | Seq. ID No. 6326 |
| 2995b | Seq. ID No. 6331 | Seq. ID No. 6332 |
| 2998a | Seq. ID No. 6335 | Seq. ID No. 6336 |
| 2999 | Seq. ID No. 6339 | Seq. ID No. 6340 |
| 3001 | Seq. ID No. 6341 | Seq. ID No. 6342 |
| 3003 | Seq. ID No. 6343 | Seq. ID No. 6344 |
| 3005 | Seq. ID No. 6345 | Seq. ID No. 6346 |
| 3008a | Seq. ID No. 6347 | Seq. ID No. 6348 |
| 3010a | Seq. ID No. 6355 | Seq. ID No. 6356 |
| 3012 | Seq. ID No. 6359 | Seq. ID No. 6360 |
| 3015 | Seq. ID No. 6361 | Seq. ID No. 6362 |
| 3016b | Seq. ID No. 6365 | Seq. ID No. 6366 |
| 3017 | Seq. ID No. 6369 | Seq. ID No. 6370 |
| 3018a | Seq. ID No. 6371 | Seq. ID No. 6372 |
| 3020 | Seq. ID No. 6375 | Seq. ID No. 6376 |
| 3021 | Seq. ID No. 6377 | Seq. ID No. 6378 |
| 3022 | Seq. ID No. 6379 | Seq. ID No. 6380 |
| 3026a | Seq. ID No. 6387 | Seq. ID No. 6388 |
| 3028a | Seq. ID No. 6391 | Seq. ID No. 6392 |
| 3029a | Seq. ID No. 6397 | Seq. ID No. 6398 |
| 3031a | Seq. ID No. 6401 | Seq. ID No. 6402 |
| 3032a | Seq. ID No. 6405 | Seq. ID No. 6406 |
| 3033a | Seq. ID No. 6409 | Seq. ID No. 6410 |
| 3035 | Seq. ID No. 6413 | Seq. ID No. 6414 |
| 3037b | Seq. ID No. 6417 | Seq. ID No. 6418 |
| 3038 | Seq. ID No. 6419 | Seq. ID No. 6420 |
| 3040b | Seq. ID No. 6423 | Seq. ID No. 6424 |
| 3043a | Seq. ID No. 6429 | Seq. ID No. 6430 |
| 3044 | Seq. ID No. 6433 | Seq. ID No. 6434 |
| 3045a | Seq. ID No. 6435 | Seq. ID No. 6436 |
| 3048a | Seq. ID No. 6439 | Seq. ID No. 6440 |
| 3049c | Seq. ID No. 6445 | Seq. ID No. 6446 |
| 3051a | Seq. ID No. 6447 | Seq. ID No. 6448 |
| 3052 | Seq. ID No. 6451 | Seq. ID No. 6452 |
| 3055a | Seq. ID No. 6455 | Seq. ID No. 6456 |
| 3057 | Seq. ID No. 6459 | Seq. ID No. 6460 |
| 3059 | Seq. ID No. 6461 | Seq. ID No. 6462 |
| 3060 | Seq. ID No. 6463 | Seq. ID No. 6464 |
| 3065a | Seq. ID No. 6475 | Seq. ID No. 6476 |
| 3067 | Seq. ID No. 6481 | Seq. ID No. 6482 |
| 3068 | Seq. ID No. 6483 | Seq. ID No. 6484 |
| 3069 | Seq. ID No. 6485 | Seq. ID No. 6486 |
| 3071 | Seq. ID No. 6487 | Seq. ID No. 6488 |
| 3072a | Seq. ID No. 6489 | Seq. ID No. 6490 |
| 3073 | Seq. ID No. 6491 | Seq. ID No. 6492 |
| 3074 | Seq. ID No. 6493 | Seq. ID No. 6494 |
| 3075 | Seq. ID No. 6495 | Seq. ID No. 6496 |
| 3077a | Seq. ID No. 6499 | Seq. ID No. 6500 |
| 3078 | Seq. ID No. 6505 | Seq. ID No. 6506 |
| 3081a | Seq. ID No. 6509 | Seq. ID No. 6510 |
| 3083c | Seq. ID No. 6513 | Seq. ID No. 6514 |
| 3086 | Seq. ID No. 6519 | Seq. ID No. 6520 |
| 3087a | Seq. ID No. 6521 | Seq. ID No. 6522 |
| 3088 | Seq. ID No. 6525 | Seq. ID No. 6526 |
| 3089a | Seq. ID No. 6527 | Seq. ID No. 6528 |
| 3090 | Seq. ID No. 6531 | Seq. ID No. 6532 |
| 3091 | Seq. ID No. 6533 | Seq. ID No. 6534 |
| 3092a | Seq. ID No. 6535 | Seq. ID No. 6536 |
| 3097 | Seq. ID No. 6545 | Seq. ID No. 6546 |
| 3098c | Seq. ID No. 6551 | Seq. ID No. 6552 |
| 3099 | Seq. ID No. 6553 | Seq. ID No. 6554 |
| 3101 | Seq. ID No. 6559 | Seq. ID No. 6560 |
| 3102 | Seq. ID No. 6561 | Seq. ID No. 6562 |
| 3103b | Seq. ID No. 6563 | Seq. ID No. 6564 |
| 3104 | Seq. ID No. 6565 | Seq. ID No. 6566 |
| 3105 | Seq. ID No. 6567 | Seq. ID No. 6568 |
| 3106 | Seq. ID No. 6569 | Seq. ID No. 6570 |
| 3108a | Seq. ID No. 6575 | Seq. ID No. 6576 |
| 3109 | Seq. ID No. 6579 | Seq. ID No. 6580 |
| 3110b | Seq. ID No. 6583 | Seq. ID No. 6584 |
| 3113 | Seq. ID No. 6585 | Seq. ID No. 6586 |
| 3118b | Seq. ID No. 6591 | Seq. ID No. 6592 |
| 3119b | Seq. ID No. 6595 | Seq. ID No. 6596 |
| 3121 | Seq. ID No. 6605 | Seq. ID No. 6606 |
| 3122a | Seq. ID No. 6607 | Seq. ID No. 6608 |
| 3123a | Seq. ID No. 6611 | Seq. ID No. 6612 |
| 3124 | Seq. ID No. 6615 | Seq. ID No. 6616 |
| 3126 | Seq. ID No. 6617 | Seq. ID No. 6618 |
| 3129a | Seq. ID No. 6619 | Seq. ID No. 6620 |
| 3131 | Seq. ID No. 6623 | Seq. ID No. 6624 |
| 3134a | Seq. ID No. 6625 | Seq. ID No. 6626 |
| 3138 | Seq. ID No. 6629 | Seq. ID No. 6630 |
| 3141 | Seq. ID No. 6633 | Seq. ID No. 6634 |
| 3145a | Seq. ID No. 6635 | Seq. ID No. 6636 |
| 3147a | Seq. ID No. 6641 | Seq. ID No. 6642 |
| 3149a | Seq. ID No. 6647 | Seq. ID No. 6648 |

Listed in Table 20 are 2343 ORFs detected by the ORF finder program of Applicant's assignee that searches for one of the three potential Start codons between a Stop-Stop region. It first looks for a ‘ATG’ Start codon but will accept either a ‘GTG’ or ‘TTG’ Start codon if found first.

TABLE 20
WYETH ORFs

| ORF Number | DNA SEQ ID Number | Protein SEQ ID Number |
|---|---|---|
| 1 | Seq. ID No. 1 | Seq. ID No. 2 |
| 2b | Seq. ID No. 5 | Seq. ID No. 6 |
| 4c | Seq. ID No. 11 | Seq. ID No. 12 |
| 5 | Seq. ID No. 13 | Seq. ID No. 14 |
| 7 | Seq. ID No. 15 | Seq. ID No. 16 |
| 10 | Seq. ID No. 17 | Seq. ID No. 18 |
| 11 | Seq. ID No. 19 | Seq. ID No. 20 |
| 13a | Seq. ID No. 21 | Seq. ID No. 22 |
| 16 | Seq. ID No. 23 | Seq. ID No. 24 |
| 18 | Seq. ID No. 27 | Seq. ID No. 28 |
| 20 | Seq. ID No. 29 | Seq. ID No. 30 |
| 21b | Seq. ID No. 31 | Seq. ID No. 32 |
| 23b | Seq. ID No. 35 | Seq. ID No. 36 |
| 24 | Seq. ID No. 37 | Seq. ID No. 38 |
| 36 | Seq. ID No. 41 | Seq. ID No. 42 |
| 37 | Seq. ID No. 43 | Seq. ID No. 44 |
| 38a | Seq. ID No. 45 | Seq. ID No. 46 |
| 38b | Seq. ID No. 47 | Seq. ID No. 48 |
| 39a | Seq. ID No. 49 | Seq. ID No. 50 |
| 39b | Seq. ID No. 51 | Seq. ID No. 52 |
| 40 | Seq. ID No. 53 | Seq. ID No. 54 |
| 41a | Seq. ID No. 55 | Seq. ID No. 56 |
| 41b | Seq. ID No. 57 | Seq. ID No. 58 |
| 41c | Seq. ID No. 59 | Seq. ID No. 60 |
| 44a | Seq. ID No. 65 | Seq. ID No. 66 |
| 44b | Seq. ID No. 67 | Seq. ID No. 68 |
| 47 | Seq. ID No. 75 | Seq. ID No. 76 |
| 48a | Seq. ID No. 77 | Seq. ID No. 78 |
| 48b | Seq. ID No. 79 | Seq. ID No. 80 |
| 48c | Seq. ID No. 81 | Seq. ID No. 82 |
| 50 | Seq. ID No. 83 | Seq. ID No. 84 |
| 51c | Seq. ID No. 89 | Seq. ID No. 90 |
| 55 | Seq. ID No. 91 | Seq. ID No. 92 |
| 56b | Seq. ID No. 93 | Seq. ID No. 94 |
| 57b | Seq. ID No. 97 | Seq. ID No. 98 |
| 58 | Seq. ID No. 99 | Seq. ID No. 100 |
| 60b | Seq. ID No. 103 | Seq. ID No. 104 |
| 61 | Seq. ID No. 105 | Seq. ID No. 106 |
| 62a | Seq. ID No. 107 | Seq. ID No. 108 |
| 62b | Seq. ID No. 109 | Seq. ID No. 110 |
| 62c | Seq. ID No. 111 | Seq. ID No. 112 |
| 63a | Seq. ID No. 113 | Seq. ID No. 114 |
| 63b | Seq. ID No. 115 | Seq. ID No. 116 |
| 65a | Seq. ID No. 117 | Seq. ID No. 118 |
| 65b | Seq. ID No. 119 | Seq. ID No. 120 |
| 65c | Seq. ID No. 121 | Seq. ID No. 122 |
| 69 | Seq. ID No. 127 | Seq. ID No. 128 |
| 71 | Seq. ID No. 129 | Seq. ID No. 130 |
| 73a | Seq. ID No. 131 | Seq. ID No. 132 |
| 73b | Seq. ID No. 133 | Seq. ID No. 134 |
| 74 | Seq. ID No. 135 | Seq. ID No. 136 |
| 77 | Seq. ID No. 137 | Seq. ID No. 138 |
| 79 | Seq. ID No. 139 | Seq. ID No. 140 |
| 80b | Seq. ID No. 143 | Seq. ID No. 144 |
| 80c | Seq. ID No. 145 | Seq. ID No. 146 |
| 81a | Seq. ID No. 147 | Seq. ID No. 148 |
| 81b | Seq. ID No. 149 | Seq. ID No. 150 |
| 85a | Seq. ID No. 153 | Seq. ID No. 154 |
| 85b | Seq. ID No. 155 | Seq. ID No. 156 |
| 85c | Seq. ID No. 157 | Seq. ID No. 158 |
| 86 | Seq. ID No. 159 | Seq. ID No. 160 |
| 87a | Seq. ID No. 161 | Seq. ID No. 162 |
| 87b | Seq. ID No. 163 | Seq. ID No. 164 |
| 87c | Seq. ID No. 165 | Seq. ID No. 166 |
| 88 | Seq. ID No. 167 | Seq. ID No. 168 |
| 89 | Seq. ID No. 169 | Seq. ID No. 170 |
| 91a | Seq. ID No. 171 | Seq. ID No. 172 |
| 91c | Seq. ID No. 175 | Seq. ID No. 176 |
| 91d | Seq. ID No. 177 | Seq. ID No. 178 |
| 93a | Seq. ID No. 179 | Seq. ID No. 180 |
| 93b | Seq. ID No. 181 | Seq. ID No. 182 |
| 93c | Seq. ID No. 183 | Seq. ID No. 184 |
| 95b | Seq. ID No. 187 | Seq. ID No. 188 |
| 99 | Seq. ID No. 191 | Seq. ID No. 192 |
| 101 | Seq. ID No. 193 | Seq. ID No. 194 |
| 102 | Seq. ID No. 195 | Seq. ID No. 196 |
| 104c | Seq. ID No. 201 | Seq. ID No. 202 |
| 105b | Seq. ID No. 205 | Seq. ID No. 206 |
| 106 | Seq. ID No. 207 | Seq. ID No. 208 |
| 109d | Seq. ID No. 215 | Seq. ID No. 216 |
| 111b | Seq. ID No. 219 | Seq. ID No. 220 |
| 112a | Seq. ID No. 221 | Seq. ID No. 222 |
| 112b | Seq. ID No. 223 | Seq. ID No. 224 |
| 114 | Seq. ID No. 225 | Seq. ID No. 226 |
| 115 | Seq. ID No. 227 | Seq. ID No. 228 |
| 119b | Seq. ID No. 231 | Seq. ID No. 232 |
| 121 | Seq. ID No. 233 | Seq. ID No. 234 |
| 124c | Seq. ID No. 239 | Seq. ID No. 240 |
| 127a | Seq. ID No. 243 | Seq. ID No. 244 |
| 127b | Seq. ID No. 245 | Seq. ID No. 246 |
| 127c | Seq. ID No. 247 | Seq. ID No. 248 |
| 129c | Seq. ID No. 253 | Seq. ID No. 254 |
| 132b | Seq. ID No. 257 | Seq. ID No. 258 |
| 132c | Seq. ID No. 259 | Seq. ID No. 260 |
| 134 | Seq. ID No. 261 | Seq. ID No. 262 |
| 135a | Seq. ID No. 263 | Seq. ID No. 264 |
| 135b | Seq. ID No. 265 | Seq. ID No. 266 |
| 137a | Seq. ID No. 267 | Seq. ID No. 268 |
| 137b | Seq. ID No. 269 | Seq. ID No. 270 |
| 138b | Seq. ID No. 273 | Seq. ID No. 274 |
| 143 | Seq. ID No. 277 | Seq. ID No. 278 |
| 144a | Seq. ID No. 279 | Seq. ID No. 280 |
| 144b | Seq. ID No. 281 | Seq. ID No. 282 |
| 144c | Seq. ID No. 283 | Seq. ID No. 284 |
| 145d | Seq. ID No. 291 | Seq. ID No. 292 |
| 147 | Seq. ID No. 293 | Seq. ID No. 294 |
| 151b | Seq. ID No. 297 | Seq. ID No. 298 |
| 152b | Seq. ID No. 301 | Seq. ID No. 302 |
| 153b | Seq. ID No. 305 | Seq. ID No. 306 |
| 153c | Seq. ID No. 307 | Seq. ID No. 308 |
| 154 | Seq. ID No. 309 | Seq. ID No. 310 |
| 155b | Seq. ID No. 313 | Seq. ID No. 314 |
| 157b | Seq. ID No. 317 | Seq. ID No. 318 |
| 158b | Seq. ID No. 321 | Seq. ID No. 322 |
| 160a | Seq. ID No. 323 | Seq. ID No. 324 |
| 160b | Seq. ID No. 325 | Seq. ID No. 326 |
| 162c | Seq. ID No. 331 | Seq. ID No. 332 |
| 162d | Seq. ID No. 333 | Seq. ID No. 334 |
| 162e | Seq. ID No. 335 | Seq. ID No. 336 |
| 163 | Seq. ID No. 337 | Seq. ID No. 338 |
| 164 | Seq. ID No. 339 | Seq. ID No. 340 |
| 165b | Seq. ID No. 343 | Seq. ID No. 344 |
| 167 | Seq. ID No. 345 | Seq. ID No. 346 |
| 169 | Seq. ID No. 347 | Seq. ID No. 348 |
| 170 | Seq. ID No. 349 | Seq. ID No. 350 |
| 171b | Seq. ID No. 353 | Seq. ID No. 354 |
| 172 | Seq. ID No. 355 | Seq. ID No. 356 |
| 173 | Seq. ID No. 357 | Seq. ID No. 358 |
| 174 | Seq. ID No. 359 | Seq. ID No. 360 |
| 175 | Seq. ID No. 361 | Seq. ID No. 362 |
| 176 | Seq. ID No. 363 | Seq. ID No. 364 |
| 177a | Seq. ID No. 365 | Seq. ID No. 366 |
| 177b | Seq. ID No. 367 | Seq. ID No. 368 |
| 179a | Seq. ID No. 369 | Seq. ID No. 370 |
| 179b | Seq. ID No. 371 | Seq. ID No. 372 |
| 180b | Seq. ID No. 375 | Seq. ID No. 376 |
| 183a | Seq. ID No. 377 | Seq. ID No. 378 |
| 184 | Seq. ID No. 379 | Seq. ID No. 380 |
| 186 | Seq. ID No. 383 | Seq. ID No. 384 |
| 187a | Seq. ID No. 385 | Seq. ID No. 386 |
| 187b | Seq. ID No. 387 | Seq. ID No. 388 |
| 188 | Seq. ID No. 389 | Seq. ID No. 390 |
| 189b | Seq. ID No. 391 | Seq. ID No. 392 |
| 191b | Seq. ID No. 393 | Seq. ID No. 394 |
| 192b | Seq. ID No. 397 | Seq. ID No. 398 |
| 194a | Seq. ID No. 401 | Seq. ID No. 402 |
| 194b | Seq. ID No. 403 | Seq. ID No. 404 |
| 195 | Seq. ID No. 405 | Seq. ID No. 406 |
| 196 | Seq. ID No. 407 | Seq. ID No. 408 |
| 197a | Seq. ID No. 409 | Seq. ID No. 410 |
| 198b | Seq. ID No. 415 | Seq. ID No. 416 |
| 203 | Seq. ID No. 423 | Seq. ID No. 424 |
| 204 | Seq. ID No. 425 | Seq. ID No. 426 |
| 205 | Seq. ID No. 427 | Seq. ID No. 428 |
| 206 | Seq. ID No. 429 | Seq. ID No. 430 |
| 207 | Seq. ID No. 431 | Seq. ID No. 432 |
| 208a | Seq. ID No. 433 | Seq. ID No. 434 |
| 208b | Seq. ID No. 435 | Seq. ID No. 436 |
| 210 | Seq. ID No. 437 | Seq. ID No. 438 |
| 211 | Seq. ID No. 439 | Seq. ID No. 440 |
| 213 | Seq. ID No. 441 | Seq. ID No. 442 |
| 215a | Seq. ID No. 443 | Seq. ID No. 444 |
| 218 | Seq. ID No. 445 | Seq. ID No. 446 |
| 219 | Seq. ID No. 447 | Seq. ID No. 448 |
| 220b | Seq. ID No. 451 | Seq. ID No. 452 |
| 220c | Seq. ID No. 453 | Seq. ID No. 454 |
| 223a | Seq. ID No. 455 | Seq. ID No. 456 |
| 223b | Seq. ID No. 457 | Seq. ID No. 458 |
| 226b | Seq. ID No. 461 | Seq. ID No. 462 |
| 227 | Seq. ID No. 463 | Seq. ID No. 464 |
| 228b | Seq. ID No. 467 | Seq. ID No. 468 |
| 228c | Seq. ID No. 469 | Seq. ID No. 470 |
| 228d | Seq. ID No. 471 | Seq. ID No. 472 |
| 232a | Seq. ID No. 473 | Seq. ID No. 474 |
| 232b | Seq. ID No. 475 | Seq. ID No. 476 |
| 235a | Seq. ID No. 477 | Seq. ID No. 478 |
| 235b | Seq. ID No. 479 | Seq. ID No. 480 |
| 236a | Seq. ID No. 481 | Seq. ID No. 482 |
| 236b | Seq. ID No. 483 | Seq. ID No. 484 |
| 236c | Seq. ID No. 485 | Seq. ID No. 486 |
| 239b | Seq. ID No. 489 | Seq. ID No. 490 |
| 239c | Seq. ID No. 491 | Seq. ID No. 492 |
| 240 | Seq. ID No. 493 | Seq. ID No. 494 |
| 241 | Seq. ID No. 495 | Seq. ID No. 496 |
| 245c | Seq. ID No. 501 | Seq. ID No. 502 |
| 246b | Seq. ID No. 505 | Seq. ID No. 506 |
| 247 | Seq. ID No. 507 | Seq. ID No. 508 |
| 248b | Seq. ID No. 511 | Seq. ID No. 512 |
| 249b | Seq. ID No. 515 | Seq. ID No. 516 |
| 251 | Seq. ID No. 517 | Seq. ID No. 518 |
| 254 | Seq. ID No. 523 | Seq. ID No. 524 |
| 255c | Seq. ID No. 529 | Seq. ID No. 530 |
| 255d | Seq. ID No. 531 | Seq. ID No. 532 |
| 258 | Seq. ID No. 533 | Seq. ID No. 534 |
| 259b | Seq. ID No. 537 | Seq. ID No. 538 |
| 259c | Seq. ID No. 539 | Seq. ID No. 540 |
| 262 | Seq. ID No. 543 | Seq. ID No. 544 |
| 263b | Seq. ID No. 547 | Seq. ID No. 548 |
| 264b | Seq. ID No. 549 | Seq. ID No. 550 |
| 265 | Seq. ID No. 551 | Seq. ID No. 552 |
| 267b | Seq. ID No. 553 | Seq. ID No. 554 |
| 267c | Seq. ID No. 555 | Seq. ID No. 556 |
| 268 | Seq. ID No. 557 | Seq. ID No. 558 |
| 269a | Seq. ID No. 559 | Seq. ID No. 560 |
| 269b | Seq. ID No. 561 | Seq. ID No. 562 |
| 271b | Seq. ID No. 565 | Seq. ID No. 566 |
| 273b | Seq. ID No. 569 | Seq. ID No. 570 |
| 277a | Seq. ID No. 571 | Seq. ID No. 572 |
| 277b | Seq. ID No. 573 | Seq. ID No. 574 |
| 277c | Seq. ID No. 575 | Seq. ID No. 576 |
| 279b | Seq. ID No. 579 | Seq. ID No. 580 |
| 280a | Seq. ID No. 581 | Seq. ID No. 582 |
| 280c | Seq. ID No. 585 | Seq. ID No. 586 |
| 280d | Seq. ID No. 587 | Seq. ID No. 588 |
| 282 | Seq. ID No. 589 | Seq. ID No. 590 |
| 283a | Seq. ID No. 591 | Seq. ID No. 592 |
| 283b | Seq. ID No. 593 | Seq. ID No. 594 |
| 284a | Seq. ID No. 595 | Seq. ID No. 596 |
| 284b | Seq. ID No. 597 | Seq. ID No. 598 |
| 285b | Seq. ID No. 599 | Seq. ID No. 600 |
| 286a | Seq. ID No. 601 | Seq. ID No. 602 |
| 286b | Seq. ID No. 603 | Seq. ID No. 604 |
| 287 | Seq. ID No. 605 | Seq. ID No. 606 |
| 290b | Seq. ID No. 609 | Seq. ID No. 610 |
| 294 | Seq. ID No. 613 | Seq. ID No. 614 |
| 296 | Seq. ID No. 615 | Seq. ID No. 616 |
| 297 | Seq. ID No. 617 | Seq. ID No. 618 |
| 300b | Seq. ID No. 619 | Seq. ID No. 620 |
| 301a | Seq. ID No. 621 | Seq. ID No. 622 |
| 301b | Seq. ID No. 623 | Seq. ID No. 624 |
| 304a | Seq. ID No. 625 | Seq. ID No. 626 |
| 304b | Seq. ID No. 627 | Seq. ID No. 628 |
| 310 | Seq. ID No. 635 | Seq. ID No. 636 |
| 311 | Seq. ID No. 637 | Seq. ID No. 638 |
| 313c | Seq. ID No. 643 | Seq. ID No. 644 |
| 313d | Seq. ID No. 645 | Seq. ID No. 646 |
| 313e | Seq. ID No. 647 | Seq. ID No. 648 |
| 315 | Seq. ID No. 649 | Seq. ID No. 650 |
| 317b | Seq. ID No. 655 | Seq. ID No. 656 |
| 318d | Seq. ID No. 663 | Seq. ID No. 664 |
| 318e | Seq. ID No. 665 | Seq. ID No. 666 |
| 320 | Seq. ID No. 667 | Seq. ID No. 668 |
| 321 | Seq. ID No. 669 | Seq. ID No. 670 |
| 324 | Seq. ID No. 675 | Seq. ID No. 676 |
| 327c | Seq. ID No. 681 | Seq. ID No. 682 |
| 329 | Seq. ID No. 683 | Seq. ID No. 684 |
| 330b | Seq. ID No. 687 | Seq. ID No. 688 |
| 330c | Seq. ID No. 689 | Seq. ID No. 690 |
| 331 | Seq. ID No. 691 | Seq. ID No. 692 |
| 333 | Seq. ID No. 695 | Seq. ID No. 696 |
| 334 | Seq. ID No. 697 | Seq. ID No. 698 |
| 335a | Seq. ID No. 699 | Seq. ID No. 700 |
| 335b | Seq. ID No. 701 | Seq. ID No. 702 |
| 337 | Seq. ID No. 703 | Seq. ID No. 704 |
| 338a | Seq. ID No. 705 | Seq. ID No. 706 |
| 338b | Seq. ID No. 707 | Seq. ID No. 708 |
| 338c | Seq. ID No. 709 | Seq. ID No. 710 |
| 341b | Seq. ID No. 713 | Seq. ID No. 714 |
| 342f | Seq. ID No. 725 | Seq. ID No. 726 |
| 342g | Seq. ID No. 727 | Seq. ID No. 728 |
| 342h | Seq. ID No. 729 | Seq. ID No. 730 |
| 344a | Seq. ID No. 731 | Seq. ID No. 732 |
| 344b | Seq. ID No. 733 | Seq. ID No. 734 |
| 345 | Seq. ID No. 735 | Seq. ID No. 736 |
| 346b | Seq. ID No. 739 | Seq. ID No. 740 |
| 347 | Seq. ID No. 741 | Seq. ID No. 742 |
| 349b | Seq. ID No. 745 | Seq. ID No. 746 |
| 349c | Seq. ID No. 747 | Seq. ID No. 748 |
| 350a | Seq. ID No. 749 | Seq. ID No. 750 |
| 350b | Seq. ID No. 751 | Seq. ID No. 752 |
| 351 | Seq. ID No. 753 | Seq. ID No. 754 |
| 352c | Seq. ID No. 759 | Seq. ID No. 760 |
| 354b | Seq. ID No. 763 | Seq. ID No. 764 |
| 354c | Seq. ID No. 765 | Seq. ID No. 766 |
| 356c | Seq. ID No. 773 | Seq. ID No. 774 |
| 356d | Seq. ID No. 775 | Seq. ID No. 776 |
| 358c | Seq. ID No. 781 | Seq. ID No. 782 |
| 362 | Seq. ID No. 783 | Seq. ID No. 784 |
| 365b | Seq. ID No. 787 | Seq. ID No. 788 |
| 367a | Seq. ID No. 793 | Seq. ID No. 794 |
| 367b | Seq. ID No. 795 | Seq. ID No. 796 |
| 371b | Seq. ID No. 799 | Seq. ID No. 800 |
| 372a | Seq. ID No. 801 | Seq. ID No. 802 |
| 372c | Seq. ID No. 805 | Seq. ID No. 806 |
| 372d | Seq. ID No. 807 | Seq. ID No. 808 |
| 374 | Seq. ID No. 809 | Seq. ID No. 810 |
| 377a | Seq. ID No. 811 | Seq. ID No. 812 |
| 377b | Seq. ID No. 813 | Seq. ID No. 814 |
| 378a | Seq. ID No. 815 | Seq. ID No. 816 |
| 378b | Seq. ID No. 817 | Seq. ID No. 818 |
| 382b | Seq. ID No. 819 | Seq. ID No. 820 |
| 383a | Seq. ID No. 821 | Seq. ID No. 822 |
| 383b | Seq. ID No. 823 | Seq. ID No. 824 |
| 384b | Seq. ID No. 827 | Seq. ID No. 828 |
| 385a | Seq. ID No. 829 | Seq. ID No. 830 |
| 385b | Seq. ID No. 831 | Seq. ID No. 832 |
| 387 | Seq. ID No. 835 | Seq. ID No. 836 |
| 390 | Seq. ID No. 837 | Seq. ID No. 838 |
| 391 | Seq. ID No. 839 | Seq. ID No. 840 |
| 395a | Seq. ID No. 843 | Seq. ID No. 844 |
| 395b | Seq. ID No. 845 | Seq. ID No. 846 |
| 395c | Seq. ID No. 847 | Seq. ID No. 848 |
| 400b | Seq. ID No. 855 | Seq. ID No. 856 |
| 401b | Seq. ID No. 859 | Seq. ID No. 860 |
| 403a | Seq. ID No. 861 | Seq. ID No. 862 |
| 403c | Seq. ID No. 865 | Seq. ID No. 866 |
| 403d | Seq. ID No. 867 | Seq. ID No. 868 |
| 406b | Seq. ID No. 871 | Seq. ID No. 872 |
| 409a | Seq. ID No. 873 | Seq. ID No. 874 |
| 409b | Seq. ID No. 875 | Seq. ID No. 876 |
| 410a | Seq. ID No. 877 | Seq. ID No. 878 |
| 410b | Seq. ID No. 879 | Seq. ID No. 880 |
| 410c | Seq. ID No. 881 | Seq. ID No. 882 |
| 413b | Seq. ID No. 885 | Seq. ID No. 886 |
| 418b | Seq. ID No. 895 | Seq. ID No. 896 |
| 419a | Seq. ID No. 897 | Seq. ID No. 898 |
| 419b | Seq. ID No. 899 | Seq. ID No. 900 |
| 419c | Seq. ID No. 901 | Seq. ID No. 902 |
| 420a | Seq. ID No. 903 | Seq. ID No. 904 |
| 420b | Seq. ID No. 905 | Seq. ID No. 906 |
| 421 | Seq. ID No. 907 | Seq. ID No. 908 |
| 422 | Seq. ID No. 909 | Seq. ID No. 910 |
| 424b | Seq. ID No. 913 | Seq. ID No. 914 |
| 424c | Seq. ID No. 915 | Seq. ID No. 916 |
| 427a | Seq. ID No. 921 | Seq. ID No. 922 |
| 427b | Seq. ID No. 923 | Seq. ID No. 924 |
| 427c | Seq. ID No. 925 | Seq. ID No. 926 |
| 429c | Seq. ID No. 931 | Seq. ID No. 932 |
| 429d | Seq. ID No. 933 | Seq. ID No. 934 |
| 431 | Seq. ID No. 935 | Seq. ID No. 936 |
| 434 | Seq. ID No. 937 | Seq. ID No. 938 |
| 435 | Seq. ID No. 939 | Seq. ID No. 940 |
| 437b | Seq. ID No. 941 | Seq. ID No. 942 |
| 437c | Seq. ID No. 943 | Seq. ID No. 944 |
| 438b | Seq. ID No. 947 | Seq. ID No. 948 |
| 438c | Seq. ID No. 949 | Seq. ID No. 950 |
| 440 | Seq. ID No. 951 | Seq. ID No. 952 |
| 441a | Seq. ID No. 953 | Seq. ID No. 954 |
| 441b | Seq. ID No. 955 | Seq. ID No. 956 |
| 443b | Seq. ID No. 957 | Seq. ID No. 958 |
| 444b | Seq. ID No. 961 | Seq. ID No. 962 |
| 445a | Seq. ID No. 963 | Seq. ID No. 964 |
| 445b | Seq. ID No. 965 | Seq. ID No. 966 |
| 448 | Seq. ID No. 967 | Seq. ID No. 968 |
| 450a | Seq. ID No. 969 | Seq. ID No. 970 |
| 450b | Seq. ID No. 971 | Seq. ID No. 972 |
| 451a | Seq. ID No. 973 | Seq. ID No. 974 |
| 451b | Seq. ID No. 975 | Seq. ID No. 976 |
| 452c | Seq. ID No. 981 | Seq. ID No. 982 |
| 452d | Seq. ID No. 983 | Seq. ID No. 984 |
| 452e | Seq. ID No. 985 | Seq. ID No. 986 |
| 454b | Seq. ID No. 987 | Seq. ID No. 988 |
| 455a | Seq. ID No. 989 | Seq. ID No. 990 |
| 455b | Seq. ID No. 991 | Seq. ID No. 992 |
| 455c | Seq. ID No. 993 | Seq. ID No. 994 |
| 458a | Seq. ID No. 995 | Seq. ID No. 996 |
| 458b | Seq. ID No. 997 | Seq. ID No. 998 |
| 460b | Seq. ID No. 1001 | Seq. ID No. 1002 |
| 460c | Seq. ID No. 1003 | Seq. ID No. 1004 |
| 462 | Seq. ID No. 1005 | Seq. ID No. 1006 |
| 463a | Seq. ID No. 1007 | Seq. ID No. 1008 |
| 463b | Seq. ID No. 1009 | Seq. ID No. 1010 |
| 464d | Seq. ID No. 1017 | Seq. ID No. 1018 |
| 465b | Seq. ID No. 1019 | Seq. ID No. 1020 |
| 467b | Seq. ID No. 1023 | Seq. ID No. 1024 |
| 471a | Seq. ID No. 1025 | Seq. ID No. 1026 |
| 471b | Seq. ID No. 1027 | Seq. ID No. 1028 |
| 472f | Seq. ID No. 1039 | Seq. ID No. 1040 |
| 473 | Seq. ID No. 1041 | Seq. ID No. 1042 |
| 474 | Seq. ID No. 1043 | Seq. ID No. 1044 |
| 476 | Seq. ID No. 1045 | Seq. ID No. 1046 |
| 477b | Seq. ID No. 1047 | Seq. ID No. 1048 |
| 478a | Seq. ID No. 1049 | Seq. ID No. 1050 |
| 478b | Seq. ID No. 1051 | Seq. ID No. 1052 |
| 480b | Seq. ID No. 1057 | Seq. ID No. 1058 |
| 481a | Seq. ID No. 1059 | Seq. ID No. 1060 |
| 481b | Seq. ID No. 1061 | Seq. ID No. 1062 |
| 482b | Seq. ID No. 1065 | Seq. ID No. 1066 |
| 490 | Seq. ID No. 1069 | Seq. ID No. 1070 |
| 492 | Seq. ID No. 1075 | Seq. ID No. 1076 |
| 494a | Seq. ID No. 1077 | Seq. ID No. 1078 |
| 495a | Seq. ID No. 1079 | Seq. ID No. 1080 |
| 495b | Seq. ID No. 1081 | Seq. ID No. 1082 |
| 495c | Seq. ID No. 1083 | Seq. ID No. 1084 |
| 498a | Seq. ID No. 1085 | Seq. ID No. 1086 |
| 498b | Seq. ID No. 1087 | Seq. ID No. 1088 |
| 499b | Seq. ID No. 1091 | Seq. ID No. 1092 |
| 500 | Seq. ID No. 1093 | Seq. ID No. 1094 |
| 502b | Seq. ID No. 1095 | Seq. ID No. 1096 |
| 502c | Seq. ID No. 1097 | Seq. ID No. 1098 |
| 504 | Seq. ID No. 1099 | Seq. ID No. 1100 |
| 505a | Seq. ID No. 1101 | Seq. ID No. 1102 |
| 505b | Seq. ID No. 1103 | Seq. ID No. 1104 |
| 507b | Seq. ID No. 1107 | Seq. ID No. 1108 |
| 509 | Seq. ID No. 1109 | Seq. ID No. 1110 |
| 510 | Seq. ID No. 1111 | Seq. ID No. 1112 |
| 512a | Seq. ID No. 1113 | Seq. ID No. 1114 |
| 512b | Seq. ID No. 1115 | Seq. ID No. 1116 |
| 515a | Seq. ID No. 1119 | Seq. ID No. 1120 |
| 515b | Seq. ID No. 1121 | Seq. ID No. 1122 |
| 515c | Seq. ID No. 1123 | Seq. ID No. 1124 |
| 518a | Seq. ID No. 1125 | Seq. ID No. 1126 |
| 518b | Seq. ID No. 1127 | Seq. ID No. 1128 |
| 518c | Seq. ID No. 1129 | Seq. ID No. 1130 |
| 521b | Seq. ID No. 1131 | Seq. ID No. 1132 |
| 524b | Seq. ID No. 1137 | Seq. ID No. 1138 |
| 525 | Seq. ID No. 1139 | Seq. ID No. 1140 |
| 527a | Seq. ID No. 1141 | Seq. ID No. 1142 |
| 527b | Seq. ID No. 1143 | Seq. ID No. 1144 |
| 528a | Seq. ID No. 1145 | Seq. ID No. 1146 |
| 528b | Seq. ID No. 1147 | Seq. ID No. 1148 |
| 529 | Seq. ID No. 1149 | Seq. ID No. 1150 |
| 530a | Seq. ID No. 1151 | Seq. ID No. 1152 |
| 530b | Seq. ID No. 1153 | Seq. ID No. 1154 |
| 533 | Seq. ID No. 1155 | Seq. ID No. 1156 |
| 535 | Seq. ID No. 1157 | Seq. ID No. 1158 |
| 536 | Seq. ID No. 1159 | Seq. ID No. 1160 |
| 539b | Seq. ID No. 1161 | Seq. ID No. 1162 |
| 539c | Seq. ID No. 1163 | Seq. ID No. 1164 |
| 541d | Seq. ID No. 1171 | Seq. ID No. 1172 |
| 543a | Seq. ID No. 1175 | Seq. ID No. 1176 |
| 543b | Seq. ID No. 1177 | Seq. ID No. 1178 |
| 543c | Seq. ID No. 1179 | Seq. ID No. 1180 |
| 544 | Seq. ID No. 1181 | Seq. ID No. 1182 |
| 545c | Seq. ID No. 1187 | Seq. ID No. 1188 |
| 546b | Seq. ID No. 1191 | Seq. ID No. 1192 |
| 546c | Seq. ID No. 1193 | Seq. ID No. 1194 |
| 547a | Seq. ID No. 1195 | Seq. ID No. 1196 |
| 547b | Seq. ID No. 1197 | Seq. ID No. 1198 |
| 549b | Seq. ID No. 1201 | Seq. ID No. 1202 |
| 550a | Seq. ID No. 1203 | Seq. ID No. 1204 |
| 551 | Seq. ID No. 1205 | Seq. ID No. 1206 |
| 553c | Seq. ID No. 1207 | Seq. ID No. 1208 |
| 554b | Seq. ID No. 1211 | Seq. ID No. 1212 |
| 556 | Seq. ID No. 1213 | Seq. ID No. 1214 |
| 557a | Seq. ID No. 1215 | Seq. ID No. 1216 |
| 557b | Seq. ID No. 1217 | Seq. ID No. 1218 |
| 560b | Seq. ID No. 1221 | Seq. ID No. 1222 |
| 561 | Seq. ID No. 1223 | Seq. ID No. 1224 |
| 563 | Seq. ID No. 1225 | Seq. ID No. 1226 |
| 564c | Seq. ID No. 1231 | Seq. ID No. 1232 |
| 569 | Seq. ID No. 1233 | Seq. ID No. 1234 |
| 570b | Seq. ID No. 1237 | Seq. ID No. 1238 |
| 571 | Seq. ID No. 1239 | Seq. ID No. 1240 |
| 573 | Seq. ID No. 1241 | Seq. ID No. 1242 |
| 576b | Seq. ID No. 1245 | Seq. ID No. 1246 |
| 578a | Seq. ID No. 1247 | Seq. ID No. 1248 |
| 578b | Seq. ID No. 1249 | Seq. ID No. 1250 |
| 580a | Seq. ID No. 1251 | Seq. ID No. 1252 |
| 580b | Seq. ID No. 1253 | Seq. ID No. 1254 |
| 583a | Seq. ID No. 1255 | Seq. ID No. 1256 |
| 583b | Seq. ID No. 1257 | Seq. ID No. 1258 |
| 585b | Seq. ID No. 1259 | Seq. ID No. 1260 |
| 586a | Seq. ID No. 1261 | Seq. ID No. 1262 |
| 586b | Seq. ID No. 1263 | Seq. ID No. 1264 |
| 587d | Seq. ID No. 1271 | Seq. ID No. 1272 |
| 588a | Seq. ID No. 1273 | Seq. ID No. 1274 |
| 588b | Seq. ID No. 1275 | Seq. ID No. 1276 |
| 589a | Seq. ID No. 1277 | Seq. ID No. 1278 |
| 589b | Seq. ID No. 1279 | Seq. ID No. 1280 |
| 595b | Seq. ID No. 1285 | Seq. ID No. 1286 |
| 596b | Seq. ID No. 1289 | Seq. ID No. 1290 |
| 596c | Seq. ID No. 1291 | Seq. ID No. 1292 |
| 597 | Seq. ID No. 1293 | Seq. ID No. 1294 |
| 598a | Seq. ID No. 1295 | Seq. ID No. 1296 |
| 598b | Seq. ID No. 1297 | Seq. ID No. 1298 |
| 599a | Seq. ID No. 1299 | Seq. ID No. 1300 |
| 599b | Seq. ID No. 1301 | Seq. ID No. 1302 |
| 600a | Seq. ID No. 1303 | Seq. ID No. 1304 |
| 600b | Seq. ID No. 1305 | Seq. ID No. 1306 |
| 603a | Seq. ID No. 1307 | Seq. ID No. 1308 |
| 606 | Seq. ID No. 1309 | Seq. ID No. 1310 |
| 607a | Seq. ID No. 1311 | Seq. ID No. 1312 |
| 607b | Seq. ID No. 1313 | Seq. ID No. 1314 |
| 609b | Seq. ID No. 1317 | Seq. ID No. 1318 |
| 610a | Seq. ID No. 1319 | Seq. ID No. 1320 |
| 610b | Seq. ID No. 1321 | Seq. ID No. 1322 |
| 613d | Seq. ID No. 1331 | Seq. ID No. 1332 |
| 614a | Seq. ID No. 1333 | Seq. ID No. 1334 |
| 614b | Seq. ID No. 1335 | Seq. ID No. 1336 |
| 615b | Seq. ID No. 1337 | Seq. ID No. 1338 |
| 618 | Seq. ID No. 1339 | Seq. ID No. 1340 |
| 619b | Seq. ID No. 1343 | Seq. ID No. 1344 |
| 620 | Seq. ID No. 1345 | Seq. ID No. 1346 |
| 623 | Seq. ID No. 1347 | Seq. ID No. 1348 |
| 624a | Seq. ID No. 1349 | Seq. ID No. 1350 |
| 624b | Seq. ID No. 1351 | Seq. ID No. 1352 |
| 626b | Seq. ID No. 1359 | Seq. ID No. 1360 |
| 626c | Seq. ID No. 1361 | Seq. ID No. 1362 |
| 631a | Seq. ID No. 1363 | Seq. ID No. 1364 |
| 631b | Seq. ID No. 1365 | Seq. ID No. 1366 |
| 631d | Seq. ID No. 1369 | Seq. ID No. 1370 |
| 632b | Seq. ID No. 1371 | Seq. ID No. 1372 |
| 633b | Seq. ID No. 1375 | Seq. ID No. 1376 |
| 633c | Seq. ID No. 1377 | Seq. ID No. 1378 |
| 636 | Seq. ID No. 1381 | Seq. ID No. 1382 |
| 637a | Seq. ID No. 1383 | Seq. ID No. 1384 |
| 637b | Seq. ID No. 1385 | Seq. ID No. 1386 |
| 637c | Seq. ID No. 1387 | Seq. ID No. 1388 |
| 640 | Seq. ID No. 1389 | Seq. ID No. 1390 |
| 641a | Seq. ID No. 1391 | Seq. ID No. 1392 |
| 641b | Seq. ID No. 1393 | Seq. ID No. 1394 |
| 645 | Seq. ID No. 1401 | Seq. ID No. 1402 |
| 650 | Seq. ID No. 1403 | Seq. ID No. 1404 |
| 653a | Seq. ID No. 1407 | Seq. ID No. 1408 |
| 653b | Seq. ID No. 1409 | Seq. ID No. 1410 |
| 653c | Seq. ID No. 1411 | Seq. ID No. 1412 |
| 656 | Seq. ID No. 1413 | Seq. ID No. 1414 |
| 657c | Seq. ID No. 1415 | Seq. ID No. 1416 |
| 658b | Seq. ID No. 1419 | Seq. ID No. 1420 |
| 659a | Seq. ID No. 1421 | Seq. ID No. 1422 |
| 659b | Seq. ID No. 1423 | Seq. ID No. 1424 |
| 661 | Seq. ID No. 1425 | Seq. ID No. 1426 |
| 663 | Seq. ID No. 1427 | Seq. ID No. 1428 |
| 665 | Seq. ID No. 1429 | Seq. ID No. 1430 |
| 670 | Seq. ID No. 1433 | Seq. ID No. 1434 |
| 674b | Seq. ID No. 1437 | Seq. ID No. 1438 |
| 677a | Seq. ID No. 1441 | Seq. ID No. 1442 |
| 677b | Seq. ID No. 1443 | Seq. ID No. 1444 |
| 679b | Seq. ID No. 1447 | Seq. ID No. 1448 |
| 680 | Seq. ID No. 1449 | Seq. ID No. 1450 |
| 681 | Seq. ID No. 1451 | Seq. ID No. 1452 |
| 683b | Seq. ID No. 1455 | Seq. ID No. 1456 |
| 687 | Seq. ID No. 1459 | Seq. ID No. 1460 |
| 688b | Seq. ID No. 1463 | Seq. ID No. 1464 |
| 690 | Seq. ID No. 1465 | Seq. ID No. 1466 |
| 691a | Seq. ID No. 1467 | Seq. ID No. 1468 |
| 691b | Seq. ID No. 1469 | Seq. ID No. 1470 |
| 692 | Seq. ID No. 1471 | Seq. ID No. 1472 |
| 694 | Seq. ID No. 1473 | Seq. ID No. 1474 |
| 696 | Seq. ID No. 1475 | Seq. ID No. 1476 |
| 698 | Seq. ID No. 1477 | Seq. ID No. 1478 |
| 699b | Seq. ID No. 1479 | Seq. ID No. 1480 |
| 700b | Seq. ID No. 1483 | Seq. ID No. 1484 |
| 700c | Seq. ID No. 1485 | Seq. ID No. 1486 |
| 703 | Seq. ID No. 1487 | Seq. ID No. 1488 |
| 705b | Seq. ID No. 1491 | Seq. ID No. 1492 |
| 707 | Seq. ID No. 1493 | Seq. ID No. 1494 |
| 709 | Seq. ID No. 1495 | Seq. ID No. 1496 |
| 710 | Seq. ID No. 1497 | Seq. ID No. 1498 |
| 712a | Seq. ID No. 1499 | Seq. ID No. 1500 |
| 712b | Seq. ID No. 1501 | Seq. ID No. 1502 |
| 715b | Seq. ID No. 1505 | Seq. ID No. 1506 |
| 717 | Seq. ID No. 1507 | Seq. ID No. 1508 |
| 721 | Seq. ID No. 1511 | Seq. ID No. 1512 |
| 722c | Seq. ID No. 1517 | Seq. ID No. 1518 |
| 724a | Seq. ID No. 1519 | Seq. ID No. 1520 |
| 724c | Seq. ID No. 1523 | Seq. ID No. 1524 |
| 724d | Seq. ID No. 1525 | Seq. ID No. 1526 |
| 726 | Seq. ID No. 1527 | Seq. ID No. 1528 |
| 728b | Seq. ID No. 1531 | Seq. ID No. 1532 |
| 730d | Seq. ID No. 1539 | Seq. ID No. 1540 |
| 732 | Seq. ID No. 1543 | Seq. ID No. 1544 |
| 738c | Seq. ID No. 1549 | Seq. ID No. 1550 |
| 739 | Seq. ID No. 1551 | Seq. ID No. 1552 |
| 740 | Seq. ID No. 1553 | Seq. ID No. 1554 |
| 741a | Seq. ID No. 1555 | Seq. ID No. 1556 |
| 741b | Seq. ID No. 1557 | Seq. ID No. 1558 |
| 743b | Seq. ID No. 1561 | Seq. ID No. 1562 |
| 744 | Seq. ID No. 1563 | Seq. ID No. 1564 |
| 745a | Seq. ID No. 1565 | Seq. ID No. 1566 |
| 745b | Seq. ID No. 1567 | Seq. ID No. 1568 |
| 746b | Seq. ID No. 1571 | Seq. ID No. 1572 |
| 747 | Seq. ID No. 1573 | Seq. ID No. 1574 |
| 748a | Seq. ID No. 1575 | Seq. ID No. 1576 |
| 748b | Seq. ID No. 1577 | Seq. ID No. 1578 |
| 749 | Seq. ID No. 1579 | Seq. ID No. 1580 |
| 751 | Seq. ID No. 1581 | Seq. ID No. 1582 |
| 754d | Seq. ID No. 1589 | Seq. ID No. 1590 |
| 756b | Seq. ID No. 1593 | Seq. ID No. 1594 |
| 758 | Seq. ID No. 1595 | Seq. ID No. 1596 |
| 760 | Seq. ID No. 1597 | Seq. ID No. 1598 |
| 762a | Seq. ID No. 1599 | Seq. ID No. 1600 |
| 762b | Seq. ID No. 1601 | Seq. ID No. 1602 |
| 763b | Seq. ID No. 1607 | Seq. ID No. 1608 |
| 766 | Seq. ID No. 1611 | Seq. ID No. 1612 |
| 767 | Seq. ID No. 1613 | Seq. ID No. 1614 |
| 770a | Seq. ID No. 1615 | Seq. ID No. 1616 |
| 770b | Seq. ID No. 1617 | Seq. ID No. 1618 |
| 770c | Seq. ID No. 1619 | Seq. ID No. 1620 |
| 772b | Seq. ID No. 1623 | Seq. ID No. 1624 |
| 774 | Seq. ID No. 1625 | Seq. ID No. 1626 |
| 775 | Seq. ID No. 1627 | Seq. ID No. 1628 |
| 779 | Seq. ID No. 1633 | Seq. ID No. 1634 |
| 780b | Seq. ID No. 1637 | Seq. ID No. 1638 |
| 782b | Seq. ID No. 1639 | Seq. ID No. 1640 |
| 785 | Seq. ID No. 1641 | Seq. ID No. 1642 |
| 786b | Seq. ID No. 1643 | Seq. ID No. 1644 |
| 788b | Seq. ID No. 1647 | Seq. ID No. 1648 |
| 789b | Seq. ID No. 1651 | Seq. ID No. 1652 |
| 789c | Seq. ID No. 1653 | Seq. ID No. 1654 |
| 791 | Seq. ID No. 1655 | Seq. ID No. 1656 |
| 792 | Seq. ID No. 1657 | Seq. ID No. 1658 |
| 794b | Seq. ID No. 1661 | Seq. ID No. 1662 |
| 794c | Seq. ID No. 1663 | Seq. ID No. 1664 |
| 794d | Seq. ID No. 1665 | Seq. ID No. 1666 |
| 797 | Seq. ID No. 1669 | Seq. ID No. 1670 |
| 799c | Seq. ID No. 1675 | Seq. ID No. 1676 |
| 799e | Seq. ID No. 1679 | Seq. ID No. 1680 |
| 799f | Seq. ID No. 1681 | Seq. ID No. 1682 |
| 803b | Seq. ID No. 1685 | Seq. ID No. 1686 |
| 804a | Seq. ID No. 1687 | Seq. ID No. 1688 |
| 804b | Seq. ID No. 1689 | Seq. ID No. 1690 |
| 807 | Seq. ID No. 1691 | Seq. ID No. 1692 |
| 808 | Seq. ID No. 1693 | Seq. ID No. 1694 |
| 809b | Seq. ID No. 1695 | Seq. ID No. 1696 |
| 812 | Seq. ID No. 1697 | Seq. ID No. 1698 |
| 813 | Seq. ID No. 1699 | Seq. ID No. 1700 |
| 820a | Seq. ID No. 1703 | Seq. ID No. 1704 |
| 820b | Seq. ID No. 1705 | Seq. ID No. 1706 |
| 826 | Seq. ID No. 1707 | Seq. ID No. 1708 |
| 829b | Seq. ID No. 1711 | Seq. ID No. 1712 |
| 830 | Seq. ID No. 1713 | Seq. ID No. 1714 |
| 834a | Seq. ID No. 1715 | Seq. ID No. 1716 |
| 834b | Seq. ID No. 1717 | Seq. ID No. 1718 |
| 834c | Seq. ID No. 1719 | Seq. ID No. 1720 |
| 835a | Seq. ID No. 1721 | Seq. ID No. 1722 |
| 835b | Seq. ID No. 1723 | Seq. ID No. 1724 |
| 836b | Seq. ID No. 1727 | Seq. ID No. 1728 |
| 836c | Seq. ID No. 1729 | Seq. ID No. 1730 |
| 837c | Seq. ID No. 1735 | Seq. ID No. 1736 |
| 843 | Seq. ID No. 1737 | Seq. ID No. 1738 |
| 847a | Seq. ID No. 1739 | Seq. ID No. 1740 |
| 847b | Seq. ID No. 1741 | Seq. ID No. 1742 |
| 849b | Seq. ID No. 1747 | Seq. ID No. 1748 |
| 851a | Seq. ID No. 1749 | Seq. ID No. 1750 |
| 851b | Seq. ID No. 1751 | Seq. ID No. 1752 |
| 853b | Seq. ID No. 1755 | Seq. ID No. 1756 |
| 853c | Seq. ID No. 1757 | Seq. ID No. 1758 |
| 854 | Seq. ID No. 1759 | Seq. ID No. 1760 |
| 857 | Seq. ID No. 1763 | Seq. ID No. 1764 |
| 859c | Seq. ID No. 1769 | Seq. ID No. 1770 |
| 859d | Seq. ID No. 1771 | Seq. ID No. 1772 |
| 862 | Seq. ID No. 1775 | Seq. ID No. 1776 |
| 863 | Seq. ID No. 1777 | Seq. ID No. 1778 |
| 864a | Seq. ID No. 1779 | Seq. ID No. 1780 |
| 864b | Seq. ID No. 1781 | Seq. ID No. 1782 |
| 866a | Seq. ID No. 1783 | Seq. ID No. 1784 |
| 866b | Seq. ID No. 1785 | Seq. ID No. 1786 |
| 868b | Seq. ID No. 1789 | Seq. ID No. 1790 |
| 870 | Seq. ID No. 1791 | Seq. ID No. 1792 |
| 873a | Seq. ID No. 1793 | Seq. ID No. 1794 |
| 873b | Seq. ID No. 1795 | Seq. ID No. 1796 |
| 873c | Seq. ID No. 1797 | Seq. ID No. 1798 |
| 876 | Seq. ID No. 1805 | Seq. ID No. 1806 |
| 878b | Seq. ID No. 1809 | Seq. ID No. 1810 |
| 878c | Seq. ID No. 1811 | Seq. ID No. 1812 |
| 880a | Seq. ID No. 1813 | Seq. ID No. 1814 |
| 880b | Seq. ID No. 1815 | Seq. ID No. 1816 |
| 880c | Seq. ID No. 1817 | Seq. ID No. 1818 |
| 885 | Seq. ID No. 1819 | Seq. ID No. 1820 |
| 886 | Seq. ID No. 1821 | Seq. ID No. 1822 |
| 887 | Seq. ID No. 1823 | Seq. ID No. 1824 |
| 888 | Seq. ID No. 1825 | Seq. ID No. 1826 |
| 890b | Seq. ID No. 1829 | Seq. ID No. 1830 |
| 904 | Seq. ID No. 1833 | Seq. ID No. 1834 |
| 908b | Seq. ID No. 1841 | Seq. ID No. 1842 |
| 908c | Seq. ID No. 1843 | Seq. ID No. 1844 |
| 911 | Seq. ID No. 1845 | Seq. ID No. 1846 |
| 912 | Seq. ID No. 1847 | Seq. ID No. 1848 |
| 916 | Seq. ID No. 1849 | Seq. ID No. 1850 |
| 918b | Seq. ID No. 1853 | Seq. ID No. 1854 |
| 918c | Seq. ID No. 1855 | Seq. ID No. 1856 |
| 919 | Seq. ID No. 1857 | Seq. ID No. 1858 |
| 923 | Seq. ID No. 1859 | Seq. ID No. 1860 |
| 925a | Seq. ID No. 1861 | Seq. ID No. 1862 |
| 925b | Seq. ID No. 1863 | Seq. ID No. 1864 |
| 926 | Seq. ID No. 1865 | Seq. ID No. 1866 |
| 927 | Seq. ID No. 1867 | Seq. ID No. 1868 |
| 929b | Seq. ID No. 1871 | Seq. ID No. 1872 |
| 930 | Seq. ID No. 1873 | Seq. ID No. 1874 |
| 931 | Seq. ID No. 1875 | Seq. ID No. 1876 |
| 932 | Seq. ID No. 1877 | Seq. ID No. 1878 |
| 934b | Seq. ID No. 1881 | Seq. ID No. 1882 |
| 934c | Seq. ID No. 1883 | Seq. ID No. 1884 |
| 934d | Seq. ID No. 1885 | Seq. ID No. 1886 |
| 935 | Seq. ID No. 1887 | Seq. ID No. 1888 |
| 938 | Seq. ID No. 1889 | Seq. ID No. 1890 |
| 939c | Seq. ID No. 1895 | Seq. ID No. 1896 |
| 944b | Seq. ID No. 1903 | Seq. ID No. 1904 |
| 946 | Seq. ID No. 1905 | Seq. ID No. 1906 |
| 949a | Seq. ID No. 1907 | Seq. ID No. 1908 |
| 949c | Seq. ID No. 1911 | Seq. ID No. 1912 |
| 949d | Seq. ID No. 1913 | Seq. ID No. 1914 |
| 950 | Seq. ID No. 1915 | Seq. ID No. 1916 |
| 951b | Seq. ID No. 1919 | Seq. ID No. 1920 |
| 952b | Seq. ID No. 1923 | Seq. ID No. 1924 |
| 954 | Seq. ID No. 1925 | Seq. ID No. 1926 |
| 955a | Seq. ID No. 1927 | Seq. ID No. 1928 |
| 955b | Seq. ID No. 1929 | Seq. ID No. 1930 |
| 956 | Seq. ID No. 1931 | Seq. ID No. 1932 |
| 958b | Seq. ID No. 1935 | Seq. ID No. 1936 |
| 961b | Seq. ID No. 1941 | Seq. ID No. 1942 |
| 961c | Seq. ID No. 1943 | Seq. ID No. 1944 |
| 963 | Seq. ID No. 1945 | Seq. ID No. 1946 |
| 965a | Seq. ID No. 1947 | Seq. ID No. 1948 |
| 965b | Seq. ID No. 1949 | Seq. ID No. 1950 |
| 966a | Seq. ID No. 1951 | Seq. ID No. 1952 |
| 966b | Seq. ID No. 1953 | Seq. ID No. 1954 |
| 968 | Seq. ID No. 1959 | Seq. ID No. 1960 |
| 969 | Seq. ID No. 1961 | Seq. ID No. 1962 |
| 972a | Seq. ID No. 1963 | Seq. ID No. 1964 |
| 972b | Seq. ID No. 1965 | Seq. ID No. 1966 |
| 976 | Seq. ID No. 1967 | Seq. ID No. 1968 |
| 977a | Seq. ID No. 1969 | Seq. ID No. 1970 |
| 977b | Seq. ID No. 1971 | Seq. ID No. 1972 |
| 980 | Seq. ID No. 1979 | Seq. ID No. 1980 |
| 982 | Seq. ID No. 1983 | Seq. ID No. 1984 |
| 984a | Seq. ID No. 1985 | Seq. ID No. 1986 |
| 984b | Seq. ID No. 1987 | Seq. ID No. 1988 |
| 986 | Seq. ID No. 1989 | Seq. ID No. 1990 |
| 987 | Seq. ID No. 1991 | Seq. ID No. 1992 |
| 988b | Seq. ID No. 1995 | Seq. ID No. 1996 |
| 988c | Seq. ID No. 1997 | Seq. ID No. 1998 |
| 988d | Seq. ID No. 1999 | Seq. ID No. 2000 |
| 991 | Seq. ID No. 2001 | Seq. ID No. 2002 |
| 992 | Seq. ID No. 2003 | Seq. ID No. 2004 |
| 993 | Seq. ID No. 2005 | Seq. ID No. 2006 |
| 994 | Seq. ID No. 2007 | Seq. ID No. 2008 |
| 998b | Seq. ID No. 2015 | Seq. ID No. 2016 |
| 999b | Seq. ID No. 2019 | Seq. ID No. 2020 |
| 1000 | Seq. ID No. 2021 | Seq. ID No. 2022 |
| 1003a | Seq. ID No. 2025 | Seq. ID No. 2026 |
| 1003b | Seq. ID No. 2027 | Seq. ID No. 2028 |
| 1004a | Seq. ID No. 2029 | Seq. ID No. 2030 |
| 1004b | Seq. ID No. 2031 | Seq. ID No. 2032 |
| 1006b | Seq. ID No. 2035 | Seq. ID No. 2036 |
| 1007 | Seq. ID No. 2037 | Seq. ID No. 2038 |
| 1013a | Seq. ID No. 2041 | Seq. ID No. 2042 |
| 1013b | Seq. ID No. 2043 | Seq. ID No. 2044 |
| 1013c | Seq. ID No. 2045 | Seq. ID No. 2046 |
| 1016c | Seq. ID No. 2047 | Seq. ID No. 2048 |
| 1017c | Seq. ID No. 2053 | Seq. ID No. 2054 |
| 1017d | Seq. ID No. 2055 | Seq. ID No. 2056 |
| 1018b | Seq. ID No. 2059 | Seq. ID No. 2060 |
| 1021b | Seq. ID No. 2063 | Seq. ID No. 2064 |
| 1021c | Seq. ID No. 2065 | Seq. ID No. 2066 |
| 1024 | Seq. ID No. 2067 | Seq. ID No. 2068 |
| 1025 | Seq. ID No. 2069 | Seq. ID No. 2070 |
| 1026 | Seq. ID No. 2071 | Seq. ID No. 2072 |
| 1029 | Seq. ID No. 2073 | Seq. ID No. 2074 |
| 1033a | Seq. ID No. 2075 | Seq. ID No. 2076 |
| 1033b | Seq. ID No. 2077 | Seq. ID No. 2078 |
| 1033c | Seq. ID No. 2079 | Seq. ID No. 2080 |
| 1035a | Seq. ID No. 2081 | Seq. ID No. 2082 |
| 1035b | Seq. ID No. 2083 | Seq. ID No. 2084 |
| 1035c | Seq. ID No. 2085 | Seq. ID No. 2086 |
| 1036b | Seq. ID No. 2089 | Seq. ID No. 2090 |
| 1036c | Seq. ID No. 2091 | Seq. ID No. 2092 |
| 1037 | Seq. ID No. 2093 | Seq. ID No. 2094 |
| 1039b | Seq. ID No. 2097 | Seq. ID No. 2098 |
| 1041 | Seq. ID No. 2099 | Seq. ID No. 2100 |
| 1042a | Seq. ID No. 2101 | Seq. ID No. 2102 |
| 1042b | Seq. ID No. 2103 | Seq. ID No. 2104 |
| 1044b | Seq. ID No. 2107 | Seq. ID No. 2108 |
| 1044c | Seq. ID No. 2109 | Seq. ID No. 2110 |
| 1044d | Seq. ID No. 2111 | Seq. ID No. 2112 |
| 1047b | Seq. ID No. 2115 | Seq. ID No. 2116 |
| 1049a | Seq. ID No. 2117 | Seq. ID No. 2118 |
| 1049b | Seq. ID No. 2119 | Seq. ID No. 2120 |
| 1050c | Seq. ID No. 2125 | Seq. ID No. 2126 |
| 1050d | Seq. ID No. 2127 | Seq. ID No. 2128 |
| 1055 | Seq. ID No. 2129 | Seq. ID No. 2130 |
| 1056a | Seq. ID No. 2131 | Seq. ID No. 2132 |
| 1056b | Seq. ID No. 2133 | Seq. ID No. 2134 |
| 1057 | Seq. ID No. 2135 | Seq. ID No. 2136 |
| 1058 | Seq. ID No. 2137 | Seq. ID No. 2138 |
| 1060b | Seq. ID No. 2141 | Seq. ID No. 2142 |
| 1062 | Seq. ID No. 2143 | Seq. ID No. 2144 |
| 1063 | Seq. ID No. 2145 | Seq. ID No. 2146 |
| 1065a | Seq. ID No. 2149 | Seq. ID No. 2150 |
| 1065b | Seq. ID No. 2151 | Seq. ID No. 2152 |
| 1065c | Seq. ID No. 2153 | Seq. ID No. 2154 |
| 1066a | Seq. ID No. 2155 | Seq. ID No. 2156 |
| 1066b | Seq. ID No. 2157 | Seq. ID No. 2158 |
| 1067 | Seq. ID No. 2159 | Seq. ID No. 2160 |
| 1069 | Seq. ID No. 2161 | Seq. ID No. 2162 |
| 1070b | Seq. ID No. 2165 | Seq. ID No. 2166 |
| 1076a | Seq. ID No. 2167 | Seq. ID No. 2168 |
| 1076b | Seq. ID No. 2169 | Seq. ID No. 2170 |
| 1077e | Seq. ID No. 2179 | Seq. ID No. 2180 |
| 1079a | Seq. ID No. 2181 | Seq. ID No. 2182 |
| 1079b | Seq. ID No. 2183 | Seq. ID No. 2184 |
| 1081b | Seq. ID No. 2189 | Seq. ID No. 2190 |
| 1082 | Seq. ID No. 2191 | Seq. ID No. 2192 |
| 1086 | Seq. ID No. 2195 | Seq. ID No. 2196 |
| 1087a | Seq. ID No. 2197 | Seq. ID No. 2198 |
| 1087b | Seq. ID No. 2199 | Seq. ID No. 2200 |
| 1091a | Seq. ID No. 2201 | Seq. ID No. 2202 |
| 1091b | Seq. ID No. 2203 | Seq. ID No. 2204 |
| 1094a | Seq. ID No. 2205 | Seq. ID No. 2206 |
| 1094b | Seq. ID No. 2207 | Seq. ID No. 2208 |
| 1095a | Seq. ID No. 2209 | Seq. ID No. 2210 |
| 1095b | Seq. ID No. 2211 | Seq. ID No. 2212 |
| 1096a | Seq. ID No. 2213 | Seq. ID No. 2214 |
| 1096b | Seq. ID No. 2215 | Seq. ID No. 2216 |
| 1096c | Seq. ID No. 2217 | Seq. ID No. 2218 |
| 1098b | Seq. ID No. 2221 | Seq. ID No. 2222 |
| 1100 | Seq. ID No. 2223 | Seq. ID No. 2224 |
| 1101b | Seq. ID No. 2227 | Seq. ID No. 2228 |
| 1104 | Seq. ID No. 2229 | Seq. ID No. 2230 |
| 1108b | Seq. ID No. 2235 | Seq. ID No. 2236 |
| 1111c | Seq. ID No. 2241 | Seq. ID No. 2242 |
| 1125 | Seq. ID No. 2245 | Seq. ID No. 2246 |
| 1126a | Seq. ID No. 2247 | Seq. ID No. 2248 |
| 1126c | Seq. ID No. 2251 | Seq. ID No. 2252 |
| 1127 | Seq. ID No. 2253 | Seq. ID No. 2254 |
| 1131 | Seq. ID No. 2257 | Seq. ID No. 2258 |
| 1132 | Seq. ID No. 2259 | Seq. ID No. 2260 |
| 1135a | Seq. ID No. 2261 | Seq. ID No. 2262 |
| 1135b | Seq. ID No. 2263 | Seq. ID No. 2264 |
| 1136a | Seq. ID No. 2265 | Seq. ID No. 2266 |
| 1136b | Seq. ID No. 2267 | Seq. ID No. 2268 |
| 1138 | Seq. ID No. 2269 | Seq. ID No. 2270 |
| 1140c | Seq. ID No. 2275 | Seq. ID No. 2276 |
| 1141a | Seq. ID No. 2277 | Seq. ID No. 2278 |
| 1141b | Seq. ID No. 2279 | Seq. ID No. 2280 |
| 1144b | Seq. ID No. 2285 | Seq. ID No. 2286 |
| 1144c | Seq. ID No. 2287 | Seq. ID No. 2288 |
| 1146e | Seq. ID No. 2297 | Seq. ID No. 2298 |
| 1148 | Seq. ID No. 2299 | Seq. ID No. 2300 |
| 1149 | Seq. ID No. 2301 | Seq. ID No. 2302 |
| 1152d | Seq. ID No. 2309 | Seq. ID No. 2310 |
| 1152e | Seq. ID No. 2311 | Seq. ID No. 2312 |
| 1152g | Seq. ID No. 2315 | Seq. ID No. 2316 |
| 1155f | Seq. ID No. 2327 | Seq. ID No. 2328 |
| 1155g | Seq. ID No. 2329 | Seq. ID No. 2330 |
| 1158b | Seq. ID No. 2333 | Seq. ID No. 2334 |
| 1165 | Seq. ID No. 2343 | Seq. ID No. 2344 |
| 1166 | Seq. ID No. 2345 | Seq. ID No. 2346 |
| 1167b | Seq. ID No. 2349 | Seq. ID No. 2350 |
| 1168 | Seq. ID No. 2351 | Seq. ID No. 2352 |
| 1169c | Seq. ID No. 2357 | Seq. ID No. 2358 |
| 1171c | Seq. ID No. 2363 | Seq. ID No. 2364 |
| 1172 | Seq. ID No. 2365 | Seq. ID No. 2366 |
| 1173 | Seq. ID No. 2367 | Seq. ID No. 2368 |
| 1174 | Seq. ID No. 2369 | Seq. ID No. 2370 |
| 1175 | Seq. ID No. 2371 | Seq. ID No. 2372 |
| 1177 | Seq. ID No. 2373 | Seq. ID No. 2374 |
| 1178 | Seq. ID No. 2375 | Seq. ID No. 2376 |
| 1179b | Seq. ID No. 2379 | Seq. ID No. 2380 |
| 1179c | Seq. ID No. 2381 | Seq. ID No. 2382 |
| 1180b | Seq. ID No. 2385 | Seq. ID No. 2386 |
| 1180c | Seq. ID No. 2387 | Seq. ID No. 2388 |
| 1180e | Seq. ID No. 2391 | Seq. ID No. 2392 |
| 1182a | Seq. ID No. 2393 | Seq. ID No. 2394 |
| 1182b | Seq. ID No. 2395 | Seq. ID No. 2396 |
| 1183a | Seq. ID No. 2397 | Seq. ID No. 2398 |
| 1183b | Seq. ID No. 2399 | Seq. ID No. 2400 |
| 1184 | Seq. ID No. 2401 | Seq. ID No. 2402 |
| 1185b | Seq. ID No. 2405 | Seq. ID No. 2406 |
| 1186b | Seq. ID No. 2409 | Seq. ID No. 2410 |
| 1188b | Seq. ID No. 2413 | Seq. ID No. 2414 |
| 1188c | Seq. ID No. 2415 | Seq. ID No. 2416 |
| 1191 | Seq. ID No. 2417 | Seq. ID No. 2418 |
| 1192b | Seq. ID No. 2421 | Seq. ID No. 2422 |
| 1194 | Seq. ID No. 2423 | Seq. ID No. 2424 |
| 1196a | Seq. ID No. 2425 | Seq. ID No. 2426 |
| 1196b | Seq. ID No. 2427 | Seq. ID No. 2428 |
| 1197 | Seq. ID No. 2429 | Seq. ID No. 2430 |
| 1199b | Seq. ID No. 2435 | Seq. ID No. 2436 |
| 1200 | Seq. ID No. 2437 | Seq. ID No. 2438 |
| 1205 | Seq. ID No. 2445 | Seq. ID No. 2446 |
| 1206 | Seq. ID No. 2447 | Seq. ID No. 2448 |
| 1207 | Seq. ID No. 2449 | Seq. ID No. 2450 |
| 1208 | Seq. ID No. 2451 | Seq. ID No. 2452 |
| 1209 | Seq. ID No. 2453 | Seq. ID No. 2454 |
| 1210a | Seq. ID No. 2455 | Seq. ID No. 2456 |
| 1210b | Seq. ID No. 2457 | Seq. ID No. 2458 |
| 1212 | Seq. ID No. 2459 | Seq. ID No. 2460 |
| 1213a | Seq. ID No. 2461 | Seq. ID No. 2462 |
| 1213b | Seq. ID No. 2463 | Seq. ID No. 2464 |
| 1214 | Seq. ID No. 2465 | Seq. ID No. 2466 |
| 1215b | Seq. ID No. 2469 | Seq. ID No. 2470 |
| 1217c | Seq. ID No. 2471 | Seq. ID No. 2472 |
| 1218b | Seq. ID No. 2475 | Seq. ID No. 2476 |
| 1221a | Seq. ID No. 2481 | Seq. ID No. 2482 |
| 1221b | Seq. ID No. 2483 | Seq. ID No. 2484 |
| 1223 | Seq. ID No. 2485 | Seq. ID No. 2486 |
| 1224c | Seq. ID No. 2491 | Seq. ID No. 2492 |
| 1225 | Seq. ID No. 2493 | Seq. ID No. 2494 |
| 1226 | Seq. ID No. 2495 | Seq. ID No. 2496 |
| 1228b | Seq. ID No. 2499 | Seq. ID No. 2500 |
| 1229b | Seq. ID No. 2501 | Seq. ID No. 2502 |
| 1229c | Seq. ID No. 2503 | Seq. ID No. 2504 |
| 1230 | Seq. ID No. 2505 | Seq. ID No. 2506 |
| 1231 | Seq. ID No. 2507 | Seq. ID No. 2508 |
| 1233b | Seq. ID No. 2511 | Seq. ID No. 2512 |
| 1233c | Seq. ID No. 2513 | Seq. ID No. 2514 |
| 1234b | Seq. ID No. 2517 | Seq. ID No. 2518 |
| 1234c | Seq. ID No. 2519 | Seq. ID No. 2520 |
| 1235 | Seq. ID No. 2521 | Seq. ID No. 2522 |
| 1237 | Seq. ID No. 2525 | Seq. ID No. 2526 |
| 1240 | Seq. ID No. 2529 | Seq. ID No. 2530 |
| 1241b | Seq. ID No. 2533 | Seq. ID No. 2534 |
| 1241c | Seq. ID No. 2535 | Seq. ID No. 2536 |
| 1243 | Seq. ID No. 2537 | Seq. ID No. 2538 |
| 1244 | Seq. ID No. 2539 | Seq. ID No. 2540 |
| 1245d | Seq. ID No. 2547 | Seq. ID No. 2548 |
| 1245e | Seq. ID No. 2549 | Seq. ID No. 2550 |
| 1246a | Seq. ID No. 2551 | Seq. ID No. 2552 |
| 1246b | Seq. ID No. 2553 | Seq. ID No. 2554 |
| 1250c | Seq. ID No. 2559 | Seq. ID No. 2560 |
| 1250d | Seq. ID No. 2561 | Seq. ID No. 2562 |
| 1250e | Seq. ID No. 2563 | Seq. ID No. 2564 |
| 1251a | Seq. ID No. 2565 | Seq. ID No. 2566 |
| 1253c | Seq. ID No. 2573 | Seq. ID No. 2574 |
| 1253d | Seq. ID No. 2575 | Seq. ID No. 2576 |
| 1256 | Seq. ID No. 2577 | Seq. ID No. 2578 |
| 1257a | Seq. ID No. 2579 | Seq. ID No. 2580 |
| 1257b | Seq. ID No. 2581 | Seq. ID No. 2582 |
| 1258c | Seq. ID No. 2587 | Seq. ID No. 2588 |
| 1258d | Seq. ID No. 2589 | Seq. ID No. 2590 |
| 1260 | Seq. ID No. 2591 | Seq. ID No. 2592 |
| 1263 | Seq. ID No. 2593 | Seq. ID No. 2594 |
| 1265 | Seq. ID No. 2595 | Seq. ID No. 2596 |
| 1266b | Seq. ID No. 2599 | Seq. ID No. 2600 |
| 1267b | Seq. ID No. 2603 | Seq. ID No. 2604 |
| 1268a | Seq. ID No. 2605 | Seq. ID No. 2606 |
| 1268b | Seq. ID No. 2607 | Seq. ID No. 2608 |
| 1269 | Seq. ID No. 2609 | Seq. ID No. 2610 |
| 1270 | Seq. ID No. 2611 | Seq. ID No. 2612 |
| 1271 | Seq. ID No. 2613 | Seq. ID No. 2614 |
| 1272b | Seq. ID No. 2617 | Seq. ID No. 2618 |
| 1273 | Seq. ID No. 2619 | Seq. ID No. 2620 |
| 1275b | Seq. ID No. 2623 | Seq. ID No. 2624 |
| 1277 | Seq. ID No. 2625 | Seq. ID No. 2626 |
| 1278a | Seq. ID No. 2627 | Seq. ID No. 2628 |
| 1278b | Seq. ID No. 2629 | Seq. ID No. 2630 |
| 1279 | Seq. ID No. 2631 | Seq. ID No. 2632 |
| 1281 | Seq. ID No. 2633 | Seq. ID No. 2634 |
| 1283a | Seq. ID No. 2635 | Seq. ID No. 2636 |
| 1283b | Seq. ID No. 2637 | Seq. ID No. 2638 |
| 1283c | Seq. ID No. 2639 | Seq. ID No. 2640 |
| 1284 | Seq. ID No. 2641 | Seq. ID No. 2642 |
| 1285a | Seq. ID No. 2643 | Seq. ID No. 2644 |
| 1285b | Seq. ID No. 2645 | Seq. ID No. 2646 |
| 1287a | Seq. ID No. 2647 | Seq. ID No. 2648 |
| 1287b | Seq. ID No. 2649 | Seq. ID No. 2650 |
| 1288 | Seq. ID No. 2651 | Seq. ID No. 2652 |
| 1289 | Seq. ID No. 2653 | Seq. ID No. 2654 |
| 1290 | Seq. ID No. 2655 | Seq. ID No. 2656 |
| 1293b | Seq. ID No. 2659 | Seq. ID No. 2660 |
| 1295 | Seq. ID No. 2663 | Seq. ID No. 2664 |
| 1297b | Seq. ID No. 2667 | Seq. ID No. 2668 |
| 1300 | Seq. ID No. 2669 | Seq. ID No. 2670 |
| 1302 | Seq. ID No. 2671 | Seq. ID No. 2672 |
| 1305b | Seq. ID No. 2675 | Seq. ID No. 2676 |
| 1305c | Seq. ID No. 2677 | Seq. ID No. 2678 |
| 1308c | Seq. ID No. 2683 | Seq. ID No. 2684 |
| 1308d | Seq. ID No. 2685 | Seq. ID No. 2686 |
| 1308e | Seq. ID No. 2687 | Seq. ID No. 2688 |
| 1311 | Seq. ID No. 2689 | Seq. ID No. 2690 |
| 1314b | Seq. ID No. 2691 | Seq. ID No. 2692 |
| 1315 | Seq. ID No. 2693 | Seq. ID No. 2694 |
| 1316 | Seq. ID No. 2695 | Seq. ID No. 2696 |
| 1317 | Seq. ID No. 2697 | Seq. ID No. 2698 |
| 1318 | Seq. ID No. 2699 | Seq. ID No. 2700 |
| 1319b | Seq. ID No. 2703 | Seq. ID No. 2704 |
| 1320c | Seq. ID No. 2709 | Seq. ID No. 2710 |
| 1321 | Seq. ID No. 2711 | Seq. ID No. 2712 |
| 1322b | Seq. ID No. 2715 | Seq. ID No. 2716 |
| 1323b | Seq. ID No. 2717 | Seq. ID No. 2718 |
| 1324 | Seq. ID No. 2719 | Seq. ID No. 2720 |
| 1326b | Seq. ID No. 2723 | Seq. ID No. 2724 |
| 1326c | Seq. ID No. 2725 | Seq. ID No. 2726 |
| 1333 | Seq. ID No. 2727 | Seq. ID No. 2728 |
| 1334a | Seq. ID No. 2729 | Seq. ID No. 2730 |
| 1334b | Seq. ID No. 2731 | Seq. ID No. 2732 |
| 1335b | Seq. ID No. 2735 | Seq. ID No. 2736 |
| 1337 | Seq. ID No. 2737 | Seq. ID No. 2738 |
| 1338 | Seq. ID No. 2739 | Seq. ID No. 2740 |
| 1344a | Seq. ID No. 2749 | Seq. ID No. 2750 |
| 1344b | Seq. ID No. 2751 | Seq. ID No. 2752 |
| 1348 | Seq. ID No. 2753 | Seq. ID No. 2754 |
| 1349a | Seq. ID No. 2755 | Seq. ID No. 2756 |
| 1349c | Seq. ID No. 2759 | Seq. ID No. 2760 |
| 1353 | Seq. ID No. 2765 | Seq. ID No. 2766 |
| 1355 | Seq. ID No. 2767 | Seq. ID No. 2768 |
| 1358a | Seq. ID No. 2769 | Seq. ID No. 2770 |
| 1358b | Seq. ID No. 2771 | Seq. ID No. 2772 |
| 1359 | Seq. ID No. 2773 | Seq. ID No. 2774 |
| 1361 | Seq. ID No. 2775 | Seq. ID No. 2776 |
| 1364b | Seq. ID No. 2779 | Seq. ID No. 2780 |
| 1364c | Seq. ID No. 2781 | Seq. ID No. 2782 |
| 1365b | Seq. ID No. 2783 | Seq. ID No. 2784 |
| 1366b | Seq. ID No. 2787 | Seq. ID No. 2788 |
| 1367 | Seq. ID No. 2789 | Seq. ID No. 2790 |
| 1368 | Seq. ID No. 2791 | Seq. ID No. 2792 |
| 1371 | Seq. ID No. 2793 | Seq. ID No. 2794 |
| 1373a | Seq. ID No. 2795 | Seq. ID No. 2796 |
| 1373b | Seq. ID No. 2797 | Seq. ID No. 2798 |
| 1374 | Seq. ID No. 2799 | Seq. ID No. 2800 |
| 1375 | Seq. ID No. 2801 | Seq. ID No. 2802 |
| 1377 | Seq. ID No. 2803 | Seq. ID No. 2804 |
| 1379 | Seq. ID No. 2807 | Seq. ID No. 2808 |
| 1380 | Seq. ID No. 2809 | Seq. ID No. 2810 |
| 1381b | Seq. ID No. 2813 | Seq. ID No. 2814 |
| 1382 | Seq. ID No. 2815 | Seq. ID No. 2816 |
| 1384b | Seq. ID No. 2821 | Seq. ID No. 2822 |
| 1384c | Seq. ID No. 2823 | Seq. ID No. 2824 |
| 1386b | Seq. ID No. 2827 | Seq. ID No. 2828 |
| 1387a | Seq. ID No. 2829 | Seq. ID No. 2830 |
| 1388b | Seq. ID No. 2831 | Seq. ID No. 2832 |
| 1389a | Seq. ID No. 2833 | Seq. ID No. 2834 |
| 1393c | Seq. ID No. 2839 | Seq. ID No. 2840 |
| 1393d | Seq. ID No. 2841 | Seq. ID No. 2842 |
| 1394 | Seq. ID No. 2843 | Seq. ID No. 2844 |
| 1395a | Seq. ID No. 2845 | Seq. ID No. 2846 |
| 1396 | Seq. ID No. 2847 | Seq. ID No. 2848 |
| 1399b | Seq. ID No. 2855 | Seq. ID No. 2856 |
| 1402c | Seq. ID No. 2861 | Seq. ID No. 2862 |
| 1405 | Seq. ID No. 2863 | Seq. ID No. 2864 |
| 1407b | Seq. ID No. 2865 | Seq. ID No. 2866 |
| 1408 | Seq. ID No. 2867 | Seq. ID No. 2868 |
| 1409 | Seq. ID No. 2869 | Seq. ID No. 2870 |
| 1410 | Seq. ID No. 2871 | Seq. ID No. 2872 |
| 1411c | Seq. ID No. 2877 | Seq. ID No. 2878 |
| 1412b | Seq. ID No. 2881 | Seq. ID No. 2882 |
| 1414a | Seq. ID No. 2883 | Seq. ID No. 2884 |
| 1414b | Seq. ID No. 2885 | Seq. ID No. 2886 |
| 1414c | Seq. ID No. 2887 | Seq. ID No. 2888 |
| 1416 | Seq. ID No. 2891 | Seq. ID No. 2892 |
| 1418 | Seq. ID No. 2893 | Seq. ID No. 2894 |
| 1419 | Seq. ID No. 2895 | Seq. ID No. 2896 |
| 1420b | Seq. ID No. 2899 | Seq. ID No. 2900 |
| 1422 | Seq. ID No. 2901 | Seq. ID No. 2902 |
| 1426b | Seq. ID No. 2907 | Seq. ID No. 2908 |
| 1429a | Seq. ID No. 2915 | Seq. ID No. 2916 |
| 1429b | Seq. ID No. 2917 | Seq. ID No. 2918 |
| 1431 | Seq. ID No. 2919 | Seq. ID No. 2920 |
| 1432 | Seq. ID No. 2921 | Seq. ID No. 2922 |
| 1433 | Seq. ID No. 2923 | Seq. ID No. 2924 |
| 1434b | Seq. ID No. 2927 | Seq. ID No. 2928 |
| 1437b | Seq. ID No. 2931 | Seq. ID No. 2932 |
| 1437c | Seq. ID No. 2933 | Seq. ID No. 2934 |
| 1438 | Seq. ID No. 2935 | Seq. ID No. 2936 |
| 1439d | Seq. ID No. 2943 | Seq. ID No. 2944 |
| 1439e | Seq. ID No. 2945 | Seq. ID No. 2946 |
| 1440c | Seq. ID No. 2951 | Seq. ID No. 2952 |
| 1440d | Seq. ID No. 2953 | Seq. ID No. 2954 |
| 1442b | Seq. ID No. 2957 | Seq. ID No. 2958 |
| 1445a | Seq. ID No. 2959 | Seq. ID No. 2960 |
| 1445b | Seq. ID No. 2961 | Seq. ID No. 2962 |
| 1445c | Seq. ID No. 2963 | Seq. ID No. 2964 |
| 1446a | Seq. ID No. 2965 | Seq. ID No. 2966 |
| 1446b | Seq. ID No. 2967 | Seq. ID No. 2968 |
| 1447b | Seq. ID No. 2971 | Seq. ID No. 2972 |
| 1448 | Seq. ID No. 2973 | Seq. ID No. 2974 |
| 1449a | Seq. ID No. 2975 | Seq. ID No. 2976 |
| 1449b | Seq. ID No. 2977 | Seq. ID No. 2978 |
| 1451 | Seq. ID No. 2979 | Seq. ID No. 2980 |
| 1452b | Seq. ID No. 2983 | Seq. ID No. 2984 |
| 1453 | Seq. ID No. 2985 | Seq. ID No. 2986 |
| 1454 | Seq. ID No. 2987 | Seq. ID No. 2988 |
| 1455b | Seq. ID No. 2991 | Seq. ID No. 2992 |
| 1456 | Seq. ID No. 2993 | Seq. ID No. 2994 |
| 1457c | Seq. ID No. 2999 | Seq. ID No. 3000 |
| 1459 | Seq. ID No. 3001 | Seq. ID No. 3002 |
| 1462b | Seq. ID No. 3005 | Seq. ID No. 3006 |
| 1463b | Seq. ID No. 3009 | Seq. ID No. 3010 |
| 1463c | Seq. ID No. 3011 | Seq. ID No. 3012 |
| 1466b | Seq. ID No. 3015 | Seq. ID No. 3016 |
| 1466c | Seq. ID No. 3017 | Seq. ID No. 3018 |
| 1466d | Seq. ID No. 3019 | Seq. ID No. 3020 |
| 1467 | Seq. ID No. 3021 | Seq. ID No. 3022 |
| 1469c | Seq. ID No. 3027 | Seq. ID No. 3028 |
| 1470c | Seq. ID No. 3033 | Seq. ID No. 3034 |
| 1471 | Seq. ID No. 3035 | Seq. ID No. 3036 |
| 1472b | Seq. ID No. 3037 | Seq. ID No. 3038 |
| 1472c | Seq. ID No. 3039 | Seq. ID No. 3040 |
| 1475 | Seq. ID No. 3043 | Seq. ID No. 3044 |
| 1476 | Seq. ID No. 3045 | Seq. ID No. 3046 |
| 1477a | Seq. ID No. 3047 | Seq. ID No. 3048 |
| 1477b | Seq. ID No. 3049 | Seq. ID No. 3050 |
| 1479a | Seq. ID No. 3051 | Seq. ID No. 3052 |
| 1479b | Seq. ID No. 3053 | Seq. ID No. 3054 |
| 1479c | Seq. ID No. 3055 | Seq. ID No. 3056 |
| 1482a | Seq. ID No. 3057 | Seq. ID No. 3058 |
| 1482b | Seq. ID No. 3059 | Seq. ID No. 3060 |
| 1484c | Seq. ID No. 3067 | Seq. ID No. 3068 |
| 1486 | Seq. ID No. 3069 | Seq. ID No. 3070 |
| 1487 | Seq. ID No. 3071 | Seq. ID No. 3072 |
| 1488d | Seq. ID No. 3079 | Seq. ID No. 3080 |
| 1488f | Seq. ID No. 3083 | Seq. ID No. 3084 |
| 1488g | Seq. ID No. 3085 | Seq. ID No. 3086 |
| 1489 | Seq. ID No. 3087 | Seq. ID No. 3088 |
| 1494 | Seq. ID No. 3091 | Seq. ID No. 3092 |
| 1497 | Seq. ID No. 3093 | Seq. ID No. 3094 |
| 1498c | Seq. ID No. 3099 | Seq. ID No. 3100 |
| 1498d | Seq. ID No. 3101 | Seq. ID No. 3102 |
| 1499 | Seq. ID No. 3103 | Seq. ID No. 3104 |
| 1503a | Seq. ID No. 3109 | Seq. ID No. 3110 |
| 1503b | Seq. ID No. 3111 | Seq. ID No. 3112 |
| 1506b | Seq. ID No. 3115 | Seq. ID No. 3116 |
| 1506c | Seq. ID No. 3117 | Seq. ID No. 3118 |
| 1507a | Seq. ID No. 3119 | Seq. ID No. 3120 |
| 1507b | Seq. ID No. 3121 | Seq. ID No. 3122 |
| 1507c | Seq. ID No. 3123 | Seq. ID No. 3124 |
| 1508 | Seq. ID No. 3125 | Seq. ID No. 3126 |
| 1510b | Seq. ID No. 3129 | Seq. ID No. 3130 |
| 1510c | Seq. ID No. 3131 | Seq. ID No. 3132 |
| 1510d | Seq. ID No. 3133 | Seq. ID No. 3134 |
| 1511 | Seq. ID No. 3135 | Seq. ID No. 3136 |
| 1512 | Seq. ID No. 3137 | Seq. ID No. 3138 |
| 1513a | Seq. ID No. 3139 | Seq. ID No. 3140 |
| 1513b | Seq. ID No. 3141 | Seq. ID No. 3142 |
| 1516 | Seq. ID No. 3143 | Seq. ID No. 3144 |
| 1517b | Seq. ID No. 3147 | Seq. ID No. 3148 |
| 1517c | Seq. ID No. 3149 | Seq. ID No. 3150 |
| 1518a | Seq. ID No. 3151 | Seq. ID No. 3152 |
| 1518b | Seq. ID No. 3153 | Seq. ID No. 3154 |
| 1520 | Seq. ID No. 3155 | Seq. ID No. 3156 |
| 1522 | Seq. ID No. 3157 | Seq. ID No. 3158 |
| 1523 | Seq. ID No. 3159 | Seq. ID No. 3160 |
| 1528 | Seq. ID No. 3161 | Seq. ID No. 3162 |
| 1530b | Seq. ID No. 3165 | Seq. ID No. 3166 |
| 1533a | Seq. ID No. 3167 | Seq. ID No. 3168 |
| 1534a | Seq. ID No. 3169 | Seq. ID No. 3170 |
| 1534b | Seq. ID No. 3171 | Seq. ID No. 3172 |
| 1535a | Seq. ID No. 3173 | Seq. ID No. 3174 |
| 1537 | Seq. ID No. 3179 | Seq. ID No. 3180 |
| 1542 | Seq. ID No. 3181 | Seq. ID No. 3182 |
| 1543 | Seq. ID No. 3183 | Seq. ID No. 3184 |
| 1544 | Seq. ID No. 3185 | Seq. ID No. 3186 |
| 1547a | Seq. ID No. 3187 | Seq. ID No. 3188 |
| 1547b | Seq. ID No. 3189 | Seq. ID No. 3190 |
| 1548 | Seq. ID No. 3191 | Seq. ID No. 3192 |
| 1552 | Seq. ID No. 3193 | Seq. ID No. 3194 |
| 1553a | Seq. ID No. 3195 | Seq. ID No. 3196 |
| 1553b | Seq. ID No. 3197 | Seq. ID No. 3198 |
| 1554c | Seq. ID No. 3203 | Seq. ID No. 3204 |
| 1555 | Seq. ID No. 3205 | Seq. ID No. 3206 |
| 1556a | Seq. ID No. 3207 | Seq. ID No. 3208 |
| 1556b | Seq. ID No. 3209 | Seq. ID No. 3210 |
| 1556c | Seq. ID No. 3211 | Seq. ID No. 3212 |
| 1557c | Seq. ID No. 3217 | Seq. ID No. 3218 |
| 1563 | Seq. ID No. 3223 | Seq. ID No. 3224 |
| 1564a | Seq. ID No. 3225 | Seq. ID No. 3226 |
| 1564b | Seq. ID No. 3227 | Seq. ID No. 3228 |
| 1565 | Seq. ID No. 3229 | Seq. ID No. 3230 |
| 1569 | Seq. ID No. 3235 | Seq. ID No. 3236 |
| 1572e | Seq. ID No. 3249 | Seq. ID No. 3250 |
| 1573 | Seq. ID No. 3251 | Seq. ID No. 3252 |
| 1576 | Seq. ID No. 3253 | Seq. ID No. 3254 |
| 1577a | Seq. ID No. 3255 | Seq. ID No. 3256 |
| 1577b | Seq. ID No. 3257 | Seq. ID No. 3258 |
| 1578 | Seq. ID No. 3259 | Seq. ID No. 3260 |
| 1580b | Seq. ID No. 3263 | Seq. ID No. 3264 |
| 1581 | Seq. ID No. 3265 | Seq. ID No. 3266 |
| 1582b | Seq. ID No. 3269 | Seq. ID No. 3270 |
| 1582c | Seq. ID No. 3271 | Seq. ID No. 3272 |
| 1584 | Seq. ID No. 3273 | Seq. ID No. 3274 |
| 1585b | Seq. ID No. 3277 | Seq. ID No. 3278 |
| 1587b | Seq. ID No. 3281 | Seq. ID No. 3282 |
| 1587c | Seq. ID No. 3283 | Seq. ID No. 3284 |
| 1588 | Seq. ID No. 3285 | Seq. ID No. 3286 |
| 1591 | Seq. ID No. 3289 | Seq. ID No. 3290 |
| 1592a | Seq. ID No. 3291 | Seq. ID No. 3292 |
| 159 b | Seq. ID No. 3293 | Seq. ID No. 3294 |
| 1594b | Seq. ID No. 3299 | Seq. ID No. 3300 |
| 1595a | Seq. ID No. 3301 | Seq. ID No. 3302 |
| 1595b | Seq. ID No. 3303 | Seq. ID No. 3304 |
| 1598a | Seq. ID No. 3305 | Seq. ID No. 3306 |
| 1598b | Seq. ID No. 3307 | Seq. ID No. 3308 |
| 1600a | Seq. ID No. 3309 | Seq. ID No. 3310 |
| 1600b | Seq. ID No. 3311 | Seq. ID No. 3312 |
| 1600c | Seq. ID No. 3313 | Seq. ID No. 3314 |
| 1602 | Seq. ID No. 3315 | Seq. ID No. 3316 |
| 1606b | Seq. ID No. 3319 | Seq. ID No. 3320 |
| 1607a | Seq. ID No. 3321 | Seq. ID No. 3322 |
| 1607b | Seq. ID No. 3323 | Seq. ID No. 3324 |
| 1608 | Seq. ID No. 3325 | Seq. ID No. 3326 |
| 1612 | Seq. ID No. 3331 | Seq. ID No. 3332 |
| 1616b | Seq. ID No. 3335 | Seq. ID No. 3336 |
| 1619 | Seq. ID No. 3337 | Seq. ID No. 3338 |
| 1621 | Seq. ID No. 3339 | Seq. ID No. 3340 |
| 1622a | Seq. ID No. 3341 | Seq. ID No. 3342 |
| 1623b | Seq. ID No. 3345 | Seq. ID No. 3346 |
| 1623c | Seq. ID No. 3347 | Seq. ID No. 3348 |
| 1624 | Seq. ID No. 3349 | Seq. ID No. 3350 |
| 1625b | Seq. ID No. 3353 | Seq. ID No. 3354 |
| 1625c | Seq. ID No. 3355 | Seq. ID No. 3356 |
| 1626b | Seq. ID No. 3359 | Seq. ID No. 3360 |
| 1628a | Seq. ID No. 3361 | Seq. ID No. 3362 |
| 1628b | Seq. ID No. 3363 | Seq. ID No. 3364 |
| 1632a | Seq. ID No. 3365 | Seq. ID No. 3366 |
| 1632b | Seq. ID No. 3367 | Seq. ID No. 3368 |
| 1633a | Seq. ID No. 3369 | Seq. ID No. 3370 |
| 1633b | Seq. ID No. 3371 | Seq. ID No. 3372 |
| 1634a | Seq. ID No. 3373 | Seq. ID No. 3374 |
| 1634b | Seq. ID No. 3375 | Seq. ID No. 3376 |
| 1634c | Seq. ID No. 3377 | Seq. ID No. 3378 |
| 1639b | Seq. ID No. 3379 | Seq. ID No. 3380 |
| 1642a | Seq. ID No. 3381 | Seq. ID No. 3382 |
| 1642b | Seq. ID No. 3383 | Seq. ID No. 3384 |
| 1646e | Seq. ID No. 3397 | Seq. ID No. 3398 |
| 1646f | Seq. ID No. 3399 | Seq. ID No. 3400 |
| 1647 | Seq. ID No. 3401 | Seq. ID No. 3402 |
| 1649b | Seq. ID No. 3407 | Seq. ID No. 3408 |
| 1649c | Seq. ID No. 3409 | Seq. ID No. 3410 |
| 1652b | Seq. ID No. 3413 | Seq. ID No. 3414 |
| 1654 | Seq. ID No. 3415 | Seq. ID No. 3416 |
| 1657 | Seq. ID No. 3419 | Seq. ID No. 3420 |
| 1659c | Seq. ID No. 3425 | Seq. ID No. 3426 |
| 1660b | Seq. ID No. 3427 | Seq. ID No. 3428 |
| 1662b | Seq. ID No. 3431 | Seq. ID No. 3432 |
| 1663 | Seq. ID No. 3433 | Seq. ID No. 3434 |
| 1664 | Seq. ID No. 3435 | Seq. ID No. 3436 |
| 1668 | Seq. ID No. 3439 | Seq. ID No. 3440 |
| 1669 | Seq. ID No. 3441 | Seq. ID No. 3442 |
| 1670a | Seq. ID No. 3443 | Seq. ID No. 3444 |
| 1670b | Seq. ID No. 3445 | Seq. ID No. 3446 |
| 1670c | Seq. ID No. 3447 | Seq. ID No. 3448 |
| 1673d | Seq. ID No. 3455 | Seq. ID No. 3456 |
| 1673e | Seq. ID No. 3457 | Seq. ID No. 3458 |
| 1673f | Seq. ID No. 3459 | Seq. ID No. 3460 |
| 1674 | Seq. ID No. 3461 | Seq. ID No. 3462 |
| 1675b | Seq. ID No. 3465 | Seq. ID No. 3466 |
| 1675c | Seq. ID No. 3467 | Seq. ID No. 3468 |
| 1677 | Seq. ID No. 3471 | Seq. ID No. 3472 |
| 1679 | Seq. ID No. 3473 | Seq. ID No. 3474 |
| 1680 | Seq. ID No. 3475 | Seq. ID No. 3476 |
| 1682b | Seq. ID No. 3479 | Seq. ID No. 3480 |
| 1683b | Seq. ID No. 3483 | Seq. ID No. 3484 |
| 1684 | Seq. ID No. 3485 | Seq. ID No. 3486 |
| 1685 | Seq. ID No. 3487 | Seq. ID No. 3488 |
| 1687 | Seq. ID No. 3489 | Seq. ID No. 3490 |
| 1688c | Seq. ID No. 3495 | Seq. ID No. 3496 |
| 1689a | Seq. ID No. 3497 | Seq. ID No. 3498 |
| 1689b | Seq. ID No. 3499 | Seq. ID No. 3500 |
| 1689c | Seq. ID No. 3501 | Seq. ID No. 3502 |
| 1690b | Seq. ID No. 3505 | Seq. ID No. 3506 |
| 1691 | Seq. ID No. 3507 | Seq. ID No. 3508 |
| 1692 | Seq. ID No. 3509 | Seq. ID No. 3510 |
| 1693 | Seq. ID No. 3511 | Seq. ID No. 3512 |
| 1694a | Seq. ID No. 3513 | Seq. ID No. 3514 |
| 1694c | Seq. ID No. 3517 | Seq. ID No. 3518 |
| 1694d | Seq. ID No. 3519 | Seq. ID No. 3520 |
| 1696 | Seq. ID No. 3521 | Seq. ID No. 3522 |
| 1697b | Seq. ID No. 3525 | Seq. ID No. 3526 |
| 1699b | Seq. ID No. 3529 | Seq. ID No. 3530 |
| 1700a | Seq. ID No. 3531 | Seq. ID No. 3532 |
| 1700b | Seq. ID No. 3533 | Seq. ID No. 3534 |
| 1701a | Seq. ID No. 3535 | Seq. ID No. 3536 |
| 1701b | Seq. ID No. 3537 | Seq. ID No. 3538 |
| 1703b | Seq. ID No. 3539 | Seq. ID No. 3540 |
| 1704a | Seq. ID No. 3541 | Seq. ID No. 3542 |
| 1704b | Seq. ID No. 3543 | Seq. ID No. 3544 |
| 1705a | Seq. ID No. 3545 | Seq. ID No. 3546 |
| 1705b | Seq. ID No. 3547 | Seq. ID No. 3548 |
| 1706 | Seq. ID No. 3549 | Seq. ID No. 3550 |
| 1707a | Seq. ID No. 3551 | Seq. ID No. 3552 |
| 1707b | Seq. ID No. 3553 | Seq. ID No. 3554 |
| 1708 | Seq. ID No. 3555 | Seq. ID No. 3556 |
| 1709 | Seq. ID No. 3557 | Seq. ID No. 3558 |
| 1710a | Seq. ID No. 3559 | Seq. ID No. 3560 |
| 1710b | Seq. ID No. 3561 | Seq. ID No. 3562 |
| 1712a | Seq. ID No. 3563 | Seq. ID No. 3564 |
| 1712b | Seq. ID No. 3565 | Seq. ID No. 3566 |
| 1715a | Seq. ID No. 3567 | Seq. ID No. 3568 |
| 1715b | Seq. ID No. 3569 | Seq. ID No. 3570 |
| 1718a | Seq. ID No. 3571 | Seq. ID No. 3572 |
| 1718b | Seq. ID No. 3573 | Seq. ID No. 3574 |
| 1722 | Seq. ID No. 3575 | Seq. ID No. 3576 |
| 1723b | Seq. ID No. 3579 | Seq. ID No. 3580 |
| 1724 | Seq. ID No. 3581 | Seq. ID No. 3582 |
| 1727 | Seq. ID No. 3583 | Seq. ID No. 3584 |
| 1731 | Seq. ID No. 3587 | Seq. ID No. 3588 |
| 1733 | Seq. ID No. 3589 | Seq. ID No. 3590 |
| 1735b | Seq. ID No. 3595 | Seq. ID No. 3596 |
| 1 737a | Seq. ID No. 3597 | Seq. ID No. 3598 |
| 1737b | Seq. ID No. 3599 | Seq. ID No. 3600 |
| 1738b | Seq. ID No. 3603 | Seq. ID No. 3604 |
| 1738c | Seq. ID No. 3605 | Seq. ID No. 3606 |
| 1739 | Seq. ID No. 3607 | Seq. ID No. 3608 |
| 1741 | Seq. ID No. 3609 | Seq. ID No. 3610 |
| 1743 | Seq. ID No. 3611 | Seq. ID No. 3612 |
| 1744a | Seq. ID No. 3613 | Seq. ID No. 3614 |
| 1744b | Seq. ID No. 3615 | Seq. ID No. 3616 |
| 1745 | Seq. ID No. 3617 | Seq. ID No. 3618 |
| 1747 | Seq. ID No. 3619 | Seq. ID No. 3620 |
| 1749a | Seq. ID No. 3621 | Seq. ID No. 3622 |
| 1749b | Seq. ID No. 3623 | Seq. ID No. 3624 |
| 1752a | Seq. ID No. 3627 | Seq. ID No. 3628 |
| 1752b | Seq. ID No. 3629 | Seq. ID No. 3630 |
| 1756b | Seq. ID No. 3635 | Seq. ID No. 3636 |
| 1757a | Seq. ID No. 3637 | Seq. ID No. 3638 |
| 1757b | Seq. ID No. 3639 | Seq. ID No. 3640 |
| 1759 | Seq. ID No. 3641 | Seq. ID No. 3642 |
| 1762 | Seq. ID No. 3645 | Seq. ID No. 3646 |
| 1764a | Seq. ID No. 3647 | Seq. ID No. 3648 |
| 1764b | Seq. ID No. 3649 | Seq. ID No. 3650 |
| 1764c | Seq. ID No. 3651 | Seq. ID No. 3652 |
| 1765 | Seq. ID No. 3653 | Seq. ID No. 3654 |
| 1767a | Seq. ID No. 3657 | Seq. ID No. 3658 |
| 1767b | Seq. ID No. 3659 | Seq. ID No. 3660 |
| 1769b | Seq. ID No. 3663 | Seq. ID No. 3664 |
| 1771 | Seq. ID No. 3665 | Seq. ID No. 3666 |
| 1773 | Seq. ID No. 3667 | Seq. ID No. 3668 |
| 1775a | Seq. ID No. 3671 | Seq. ID No. 3672 |
| 1775b | Seq. ID No. 3673 | Seq. ID No. 3674 |
| 1775c | Seq. ID No. 3675 | Seq. ID No. 3676 |
| 1778a | Seq. ID No. 3677 | Seq. ID No. 3678 |
| 1778b | Seq. ID No. 3679 | Seq. ID No. 3680 |
| 1780 | Seq. ID No. 3681 | Seq. ID No. 3682 |
| 1783a | Seq. ID No. 3683 | Seq. ID No. 3684 |
| 1783b | Seq. ID No. 3685 | Seq. ID No. 3686 |
| 1784a | Seq. ID No. 3687 | Seq. ID No. 3688 |
| 1784b | Seq. ID No. 3689 | Seq. ID No. 3690 |
| 1788a | Seq. ID No. 3691 | Seq. ID No. 3692 |
| 1788b | Seq. ID No. 3693 | Seq. ID No. 3694 |
| 1788c | Seq. ID No. 3695 | Seq. ID No. 3696 |
| 1789b | Seq. ID No. 3699 | Seq. ID No. 3700 |
| 1790a | Seq. ID No. 3701 | Seq. ID No. 3702 |
| 1790b | Seq. ID No. 3703 | Seq. ID No. 3704 |
| 1792a | Seq. ID No. 3705 | Seq. ID No. 3706 |
| 1792b | Seq. ID No. 3707 | Seq. ID No. 3708 |
| 1792c | Seq. ID No. 3709 | Seq. ID No. 3710 |
| 1793b | Seq. ID No. 3713 | Seq. ID No. 3714 |
| 1793c | Seq. ID No. 3715 | Seq. ID No. 3716 |
| 1794a | Seq. ID No. 3717 | Seq. ID No. 3718 |
| 1798c | Seq. ID No. 3727 | Seq. ID No. 3728 |
| 1800 | Seq. ID No. 3729 | Seq. ID No. 3730 |
| 1802b | Seq. ID No. 3733 | Seq. ID No. 3734 |
| 1804a | Seq. ID No. 3737 | Seq. ID No. 3738 |
| 1804b | Seq. ID No. 3739 | Seq. ID No. 3740 |
| 1805 | Seq. ID No. 3741 | Seq. ID No. 3742 |
| 1806b | Seq. ID No. 3745 | Seq. ID No. 3746 |
| 1806c | Seq. ID No. 3747 | Seq. ID No. 3748 |
| 1808d | Seq. ID No. 3759 | Seq. ID No. 3760 |
| 1809d | Seq. ID No. 3767 | Seq. ID No. 3768 |
| 1809e | Seq. ID No. 3769 | Seq. ID No. 3770 |
| 1809f | Seq. ID No. 3771 | Seq. ID No. 3772 |
| 1811b | Seq. ID No. 3779 | Seq. ID No. 3780 |
| 1812b | Seq. ID No. 3783 | Seq. ID No. 3784 |
| 1813 | Seq. ID No. 3785 | Seq. ID No. 3786 |
| 1815b | Seq. ID No. 3789 | Seq. ID No. 3790 |
| 1815c | Seq. ID No. 3791 | Seq. ID No. 3792 |
| 1816b | Seq. ID No. 3795 | Seq. ID No. 3796 |
| 1816c | Seq. ID No. 3797 | Seq. ID No. 3798 |
| 1819b | Seq. ID No. 3803 | Seq. ID No. 3804 |
| 1821 | Seq. ID No. 3805 | Seq. ID No. 3806 |
| 1822 | Seq. ID No. 3807 | Seq. ID No. 3808 |
| 1823a | Seq. ID No. 3809 | Seq. ID No. 3810 |
| 1823b | Seq. ID No. 3811 | Seq. ID No. 3812 |
| 1825 | Seq. ID No. 3813 | Seq. ID No. 3814 |
| 1826a | Seq. ID No. 3815 | Seq. ID No. 3816 |
| 1826b | Seq. ID No. 3817 | Seq. ID No. 3818 |
| 1828 | Seq. ID No. 3819 | Seq. ID No. 3820 |
| 1830b | Seq. ID No. 3823 | Seq. ID No. 3824 |
| 1833 | Seq. ID No. 3825 | Seq. ID No. 3826 |
| 1836a | Seq. ID No. 3831 | Seq. ID No. 3832 |
| 1836b | Seq. ID No. 3833 | Seq. ID No. 3834 |
| 1837 | Seq. ID No. 3835 | Seq. ID No. 3836 |
| 1838c | Seq. ID No. 3841 | Seq. ID No. 3842 |
| 1838d | Seq. ID No. 3843 | Seq. ID No. 3844 |
| 1839b | Seq. ID No. 3847 | Seq. ID No. 3848 |
| 1842d | Seq. ID No. 3855 | Seq. ID No. 3856 |
| 1843 | Seq. ID No. 3857 | Seq. ID No. 3858 |
| 1845c | Seq. ID No. 3863 | Seq. ID No. 3864 |
| 1848b | Seq. ID No. 3867 | Seq. ID No. 3868 |
| 1850b | Seq. ID No. 3871 | Seq. ID No. 3872 |
| 1850c | Seq. ID No. 3873 | Seq. ID No. 3874 |
| 1853a | Seq. ID No. 3875 | Seq. ID No. 3876 |
| 1853b | Seq. ID No. 3877 | Seq. ID No. 3878 |
| 1854b | Seq. ID No. 3881 | Seq. ID No. 3882 |
| 1855 | Seq. ID No. 3883 | Seq. ID No. 3884 |
| 1856 | Seq. ID No. 3885 | Seq. ID No. 3886 |
| 1857d | Seq. ID No. 3893 | Seq. ID No. 3894 |
| 1858 | Seq. ID No. 3895 | Seq. ID No. 3896 |
| 1861a | Seq. ID No. 3899 | Seq. ID No. 3900 |
| 1861b | Seq. ID No. 3901 | Seq. ID No. 3902 |
| 1863a | Seq. ID No. 3905 | Seq. ID No. 3906 |
| 1863b | Seq. ID No. 3907 | Seq. ID No. 3908 |
| 1865a | Seq. ID No. 3911 | Seq. ID No. 3912 |
| 1865b | Seq. ID No. 3913 | Seq. ID No. 3914 |
| 1868 | Seq. ID No. 3915 | Seq. ID No. 3916 |
| 1870 | Seq. ID No. 3919 | Seq. ID No. 3920 |
| 1873a | Seq. ID No. 3921 | Seq. ID No. 3922 |
| 1873b | Seq. ID No. 3923 | Seq. ID No. 3924 |
| 1873c | Seq. ID No. 3925 | Seq. ID No. 3926 |
| 1875b | Seq. ID No. 3927 | Seq. ID No. 3928 |
| 1875c | Seq. ID No. 3929 | Seq. ID No. 3930 |
| 1877a | Seq. ID No. 3931 | Seq. ID No. 3932 |
| 1877b | Seq. ID No. 3933 | Seq. ID No. 3934 |
| 1878 | Seq. ID No. 3935 | Seq. ID No. 3936 |
| 1882 | Seq. ID No. 3939 | Seq. ID No. 3940 |
| 1884 | Seq. ID No. 3941 | Seq. ID No. 3942 |
| 1886 | Seq. ID No. 3943 | Seq. ID No. 3944 |
| 1887 | Seq. ID No. 3945 | Seq. ID No. 3946 |
| 1888a | Seq. ID No. 3947 | Seq. ID No. 3948 |
| 1888b | Seq. ID No. 3949 | Seq. ID No. 3950 |
| 1890 | Seq. ID No. 3951 | Seq. ID No. 3952 |
| 1891a | Seq. ID No. 3953 | Seq. ID No. 3954 |
| 1891b | Seq. ID No. 3955 | Seq. ID No. 3956 |
| 1893 | Seq. ID No. 3957 | Seq. ID No. 3958 |
| 1894c | Seq. ID No. 3959 | Seq. ID No. 3960 |
| 1895 | Seq. ID No. 3961 | Seq. ID No. 3962 |
| 1896b | Seq. ID No. 3965 | Seq. ID No. 3966 |
| 1897a | Seq. ID No. 3967 | Seq. ID No. 3968 |
| 1897b | Seq. ID No. 3969 | Seq. ID No. 3970 |
| 1897c | Seq. ID No. 3971 | Seq. ID No. 3972 |
| 1898b | Seq. ID No. 3975 | Seq. ID No. 3976 |
| 1900 | Seq. ID No. 3979 | Seq. ID No. 3980 |
| 1902a | Seq. ID No. 3981 | Seq. ID No. 3982 |
| 1902b | Seq. ID No. 3983 | Seq. ID No. 3984 |
| 1907 | Seq. ID No. 3985 | Seq. ID No. 3986 |
| 1912 | Seq. ID No. 3987 | Seq. ID No. 3988 |
| 1915b | Seq. ID No. 3991 | Seq. ID No. 3992 |
| 1917a | Seq. ID No. 3995 | Seq. ID No. 3996 |
| 1917b | Seq. ID No. 3997 | Seq. ID No. 3998 |
| 1918b | Seq. ID No. 3999 | Seq. ID No. 4000 |
| 1918d | Seq. ID No. 4003 | Seq. ID No. 4004 |
| 1919b | Seq. ID No. 4007 | Seq. ID No. 4008 |
| 1919c | Seq. ID No. 4009 | Seq. ID No. 4010 |
| 1925 | Seq. ID No. 4015 | Seq. ID No. 4016 |
| 1926 | Seq. ID No. 4017 | Seq. ID No. 4018 |
| 1927b | Seq. ID No. 4021 | Seq. ID No. 4022 |
| 1930 | Seq. ID No. 4023 | Seq. ID No. 4024 |
| 1931c | Seq. ID No. 4029 | Seq. ID No. 4030 |
| 1933b | Seq. ID No. 4035 | Seq. ID No. 4036 |
| 1934 | Seq. ID No. 4037 | Seq. ID No. 4038 |
| 1936 | Seq. ID No. 4039 | Seq. ID No. 4040 |
| 1937e | Seq. ID No. 4049 | Seq. ID No. 4050 |
| 1939a | Seq. ID No. 4051 | Seq. ID No. 4052 |
| 1939b | Seq. ID No. 4053 | Seq. ID No. 4054 |
| 1939c | Seq. ID No. 4055 | Seq. ID No. 4056 |
| 1940 | Seq. ID No. 4057 | Seq. ID No. 4058 |
| 1941b | Seq. ID No. 4061 | Seq. ID No. 4062 |
| 1946c | Seq. ID No. 4075 | Seq. ID No. 4076 |
| 1950 | Seq. ID No. 4079 | Seq. ID No. 4080 |
| 1951c | Seq. ID No. 4085 | Seq. ID No. 4086 |
| 1951d | Seq. ID No. 4087 | Seq. ID No. 4088 |
| 1953 | Seq. ID No. 4089 | Seq. ID No. 4090 |
| 1954a | Seq. ID No. 4091 | Seq. ID No. 4092 |
| 1954b | Seq. ID No. 4093 | Seq. ID No. 4094 |
| 1954c | Seq. ID No. 4095 | Seq. ID No. 4096 |
| 1957 | Seq. ID No. 4099 | Seq. ID No. 4100 |
| 1958c | Seq. ID No. 4105 | Seq. ID No. 4106 |
| 1960a | Seq. ID No. 4107 | Seq. ID No. 4108 |
| 1960b | Seq. ID No. 4109 | Seq. ID No. 4110 |
| 1960c | Seq. ID No. 4111 | Seq. ID No. 4112 |
| 1961 | Seq. ID No. 4113 | Seq. ID No. 4114 |
| 1962b | Seq. ID No. 4117 | Seq. ID No. 4118 |
| 1965a | Seq. ID No. 4119 | Seq. ID No. 4120 |
| 1965b | Seq. ID No. 4121 | Seq. ID No. 4122 |
| 1966 | Seq. ID No. 4123 | Seq. ID No. 4124 |
| 1967 | Seq. ID No. 4125 | Seq. ID No. 4126 |
| 1968 | Seq. ID No. 4127 | Seq. ID No. 4128 |
| 1969 | Seq. ID No. 4129 | Seq. ID No. 4130 |
| 1970a | Seq. ID No. 4131 | Seq. ID No. 4132 |
| 1970b | Seq. ID No. 4133 | Seq. ID No. 4134 |
| 1972 | Seq. ID No. 4135 | Seq. ID No. 4136 |
| 1973 | Seq. ID No. 4137 | Seq. ID No. 4138 |
| 1976 | Seq. ID No. 4139 | Seq. ID No. 4140 |
| 1977a | Seq. ID No. 4141 | Seq. ID No. 4142 |
| 1977b | Seq. ID No. 4143 | Seq. ID No. 4144 |
| 1977c | Seq. ID No. 4145 | Seq. ID No. 4146 |
| 1979b | Seq. ID No. 4149 | Seq. ID No. 4150 |
| 1980b | Seq. ID No. 4153 | Seq. ID No. 4154 |
| 1981 | Seq. ID No. 4155 | Seq. ID No. 4156 |
| 1982 | Seq. ID No. 4157 | Seq. ID No. 4158 |
| 1985b | Seq. ID No. 4161 | Seq. ID No. 4162 |
| 1986 | Seq. ID No. 4163 | Seq. ID No. 4164 |
| 1987b | Seq. ID No. 4167 | Seq. ID No. 4168 |
| 1989b | Seq. ID No. 4171 | Seq. ID No. 4172 |
| 1989c | Seq. ID No. 4173 | Seq. ID No. 4174 |
| 1990a | Seq. ID No. 4175 | Seq. ID No. 4176 |
| 1990b | Seq. ID No. 4177 | Seq. ID No. 4178 |
| 1990c | Seq. ID No. 4179 | Seq. ID No. 4180 |
| 1991 | Seq. ID No. 4181 | Seq. ID No. 4182 |
| 1992b | Seq. ID No. 4185 | Seq. ID No. 4186 |
| 1993a | Seq. ID No. 4187 | Seq. ID No. 4188 |
| 1993b | Seq. ID No. 4189 | Seq. ID No. 4190 |
| 1993c | Seq. ID No. 4191 | Seq. ID No. 4192 |
| 1996 | Seq. ID No. 4193 | Seq. ID No. 4194 |
| 2000 | Seq. ID No. 4195 | Seq. ID No. 4196 |
| 2001 | Seq. ID No. 4197 | Seq. ID No. 4198 |
| 2003 | Seq. ID No. 4199 | Seq. ID No. 4200 |
| 2008 | Seq. ID No. 4203 | Seq. ID No. 4204 |
| 2010 | Seq. ID No. 4205 | Seq. ID No. 4206 |
| 2011a | Seq. ID No. 4207 | Seq. ID No. 4208 |
| 2011b | Seq. ID No. 4209 | Seq. ID No. 4210 |
| 2011c | Seq. ID No. 4211 | Seq. ID No. 4212 |
| 2015 | Seq. ID No. 4219 | Seq. ID No. 4220 |
| 2016e | Seq. ID No. 4229 | Seq. ID No. 4230 |
| 2016g | Seq. ID No. 4233 | Seq. ID No. 4234 |
| 2016h | Seq. ID No. 4235 | Seq. ID No. 4236 |
| 2020 | Seq. ID No. 4237 | Seq. ID No. 4238 |
| 2021 | Seq. ID No. 4239 | Seq. ID No. 4240 |
| 2023b | Seq. ID No. 4243 | Seq. ID No. 4244 |
| 2024c | Seq. ID No. 4249 | Seq. ID No. 4250 |
| 2024d | Seq. ID No. 4251 | Seq. ID No. 4252 |
| 2025 | Seq. ID No. 4253 | Seq. ID No. 4254 |
| 2027 | Seq. ID No. 4255 | Seq. ID No. 4256 |
| 2030b | Seq. ID No. 4259 | Seq. ID No. 4260 |
| 2030d | Seq. ID No. 4263 | Seq. ID No. 4264 |
| 2032c | Seq. ID No. 4265 | Seq. ID No. 4266 |
| 2033 | Seq. ID No. 4267 | Seq. ID No. 4268 |
| 2035b | Seq. ID No. 4269 | Seq. ID No. 4270 |
| 2036 | Seq. ID No. 4271 | Seq. ID No. 4272 |
| 2037 | Seq. ID No. 4273 | Seq. ID No. 4274 |
| 2038a | Seq. ID No. 4275 | Seq. ID No. 4276 |
| 2043b | Seq. ID No. 4281 | Seq. ID No. 4282 |
| 2044 | Seq. ID No. 4283 | Seq. ID No. 4284 |
| 2045a | Seq. ID No. 4285 | Seq. ID No. 4286 |
| 2045b | Seq. ID No. 4287 | Seq. ID No. 4288 |
| 2046a | Seq. ID No. 4289 | Seq. ID No. 4290 |
| 2046b | Seq. ID No. 4291 | Seq. ID No. 4292 |
| 2047a | Seq. ID No. 4293 | Seq. ID No. 4294 |
| 2047b | Seq. ID No. 4295 | Seq. ID No. 4296 |
| 2049c | Seq. ID No. 4297 | Seq. ID No. 4298 |
| 2050 | Seq. ID No. 4299 | Seq. ID No. 4300 |
| 2053 | Seq. ID No. 4301 | Seq. ID No. 4302 |
| 2054 | Seq. ID No. 4303 | Seq. ID No. 4304 |
| 2056 | Seq. ID No. 4307 | Seq. ID No. 4308 |
| 2057 | Seq. ID No. 4309 | Seq. ID No. 4310 |
| 2059b | Seq. ID No. 4313 | Seq. ID No. 4314 |
| 2060a | Seq. ID No. 4315 | Seq. ID No. 4316 |
| 2060b | Seq. ID No. 4317 | Seq. ID No. 4318 |
| 2062a | Seq. ID No. 4319 | Seq. ID No. 4320 |
| 2062b | Seq. ID No. 4321 | Seq. ID No. 4322 |
| 2062c | Seq. ID No. 4323 | Seq. ID No. 4324 |
| 2064 | Seq. ID No. 4325 | Seq. ID No. 4326 |
| 2065b | Seq. ID No. 4329 | Seq. ID No. 4330 |
| 2066 | Seq. ID No. 4331 | Seq. ID No. 4332 |
| 2068 | Seq. ID No. 4333 | Seq. ID No. 4334 |
| 2069a | Seq. ID No. 4335 | Seq. ID No. 4336 |
| 2069b | Seq. ID No. 4337 | Seq. ID No. 4338 |
| 2069c | Seq. ID No. 4339 | Seq. ID No. 4340 |
| 2074b | Seq. ID No. 4343 | Seq. ID No. 4344 |
| 2075a | Seq. ID No. 4345 | Seq. ID No. 4346 |
| 2075b | Seq. ID No. 4347 | Seq. ID No. 4348 |
| 2076b | Seq. ID No. 4351 | Seq. ID No. 4352 |
| 2076c | Seq. ID No. 4353 | Seq. ID No. 4354 |
| 2078 | Seq. ID No. 4355 | Seq. ID No. 4356 |
| 2079b | Seq. ID No. 4359 | Seq. ID No. 4360 |
| 2079c | Seq. ID No. 4361 | Seq. ID No. 4362 |
| 2081a | Seq. ID No. 4363 | Seq. ID No. 4364 |
| 2081b | Seq. ID No. 4365 | Seq. ID No. 4366 |
| 2086a | Seq. ID No. 4367 | Seq. ID No. 4368 |
| 2086b | Seq. ID No. 4369 | Seq. ID No. 4370 |
| 2088 | Seq. ID No. 4375 | Seq. ID No. 4376 |
| 2091 | Seq. ID No. 4377 | Seq. ID No. 4378 |
| 2092 | Seq. ID No. 4379 | Seq. ID No. 4380 |
| 2094 | Seq. ID No. 4383 | Seq. ID No. 4384 |
| 2096c | Seq. ID No. 4389 | Seq. ID No. 4390 |
| 2096d | Seq. ID No. 4391 | Seq. ID No. 4392 |
| 2097 | Seq. ID No. 4393 | Seq. ID No. 4394 |
| 2098b | Seq. ID No. 4395 | Seq. ID No. 4396 |
| 2100 | Seq. ID No. 4397 | Seq. ID No. 4398 |
| 2101 | Seq. ID No. 4399 | Seq. ID No. 4400 |
| 2104 | Seq. ID No. 4401 | Seq. ID No. 4402 |
| 2105 | Seq. ID No. 4403 | Seq. ID No. 4404 |
| 2107a | Seq. ID No. 4405 | Seq. ID No. 4406 |
| 2107b | Seq. ID No. 4407 | Seq. ID No. 4408 |
| 2109a | Seq. ID No. 4409 | Seq. ID No. 4410 |
| 2109b | Seq. ID No. 4411 | Seq. ID No. 4412 |
| 2110a | Seq. ID No. 4413 | Seq. ID No. 4414 |
| 2110b | Seq. ID No. 4415 | Seq. ID No. 4416 |
| 2110c | Seq. ID No. 4417 | Seq. ID No. 4418 |
| 2112a | Seq. ID No. 4419 | Seq. ID No. 4420 |
| 2112b | Seq. ID No. 4421 | Seq. ID No. 4422 |
| 2112c | Seq. ID No. 4423 | Seq. ID No. 4424 |
| 2114b | Seq. ID No. 4425 | Seq. ID No. 4426 |
| 2115a | Seq. ID No. 4427 | Seq. ID No. 4428 |
| 2115b | Seq. ID No. 4429 | Seq. ID No. 4430 |
| 2115c | Seq. ID No. 4431 | Seq. ID No. 4432 |
| 2117 | Seq. ID No. 4435 | Seq. ID No. 4436 |
| 2120a | Seq. ID No. 4437 | Seq. ID No. 4438 |
| 2120b | Seq. ID No. 4439 | Seq. ID No. 4440 |
| 2122b | Seq. ID No. 4441 | Seq. ID No. 4442 |
| 2125a | Seq. ID No. 4449 | Seq. ID No. 4450 |
| 2125b | Seq. ID No. 4451 | Seq. ID No. 4452 |
| 2127a | Seq. ID No. 4453 | Seq. ID No. 4454 |
| 2127b | Seq. ID No. 4455 | Seq. ID No. 4456 |
| 2128a | Seq. ID No. 4457 | Seq. ID No. 4458 |
| 2128b | Seq. ID No. 4459 | Seq. ID No. 4460 |
| 2129a | #N/A | #N/A |
| 2129b | #N/A | #N/A |
| 2133 | Seq. ID No. 4461 | Seq. ID No. 4462 |
| 2138b | Seq. ID No. 4465 | Seq. ID No. 4466 |
| 2138c | Seq. ID No. 4467 | Seq. ID No. 4468 |
| 2140b | Seq. ID No. 4471 | Seq. ID No. 4472 |
| 2142b | Seq. ID No. 4475 | Seq. ID No. 4476 |
| 2144a | Seq. ID No. 4477 | Seq. ID No. 4478 |
| 2144b | Seq. ID No. 4479 | Seq. ID No. 4480 |
| 2146 | Seq. ID No. 4481 | Seq. ID No. 4482 |
| 2147b | Seq. ID No. 4485 | Seq. ID No. 4486 |
| 2147c | Seq. ID No. 4487 | Seq. ID No. 4488 |
| 2149a | Seq. ID No. 4489 | Seq. ID No. 4490 |
| 2149b | Seq. ID No. 4491 | Seq. ID No. 4492 |
| 2150a | Seq. ID No. 4493 | Seq. ID No. 4494 |
| 2150b | Seq. ID No. 4495 | Seq. ID No. 4496 |
| 2150c | Seq. ID No. 4497 | Seq. ID No. 4498 |
| 2153 | Seq. ID No. 4501 | Seq. ID No. 4502 |
| 2155a | Seq. ID No. 4503 | Seq. ID No. 4504 |
| 2155b | Seq. ID No. 4505 | Seq. ID No. 4506 |
| 2156a | Seq. ID No. 4507 | Seq. ID No. 4508 |
| 2156b | Seq. ID No. 4509 | Seq. ID No. 4510 |
| 2156c | Seq. ID No. 4511 | Seq. ID No. 4512 |
| 2159a | Seq. ID No. 4513 | Seq. ID No. 4514 |
| 2159b | Seq. ID No. 4515 | Seq. ID No. 4516 |
| 2160b | Seq. ID No. 4519 | Seq. ID No. 4520 |
| 2160c | Seq. ID No. 4521 | Seq. ID No. 4522 |
| 2160d | Seq. ID No. 4523 | Seq. ID No. 4524 |
| 2161a | Seq. ID No. 4525 | Seq. ID No. 4526 |
| 2161b | Seq. ID No. 4527 | Seq. ID No. 4528 |
| 2161c | Seq. ID No. 4529 | Seq. ID No. 4530 |
| 2164 | Seq. ID No. 4531 | Seq. ID No. 4532 |
| 2165 | Seq. ID No. 4533 | Seq. ID No. 4534 |
| 2166b | Seq. ID No. 4537 | Seq. ID No. 4538 |
| 2171 | Seq. ID No. 4541 | Seq. ID No. 4542 |
| 2173a | Seq. ID No. 4545 | Seq. ID No. 4546 |
| 2173b | Seq. ID No. 4547 | Seq. ID No. 4548 |
| 2174 | Seq. ID No. 4549 | Seq. ID No. 4550 |
| 2175 | Seq. ID No. 4551 | Seq. ID No. 4552 |
| 2176a | Seq. ID No. 4553 | Seq. ID No. 4554 |
| 2176b | Seq. ID No. 4555 | Seq. ID No. 4556 |
| 2176d | Seq. ID No. 4559 | Seq. ID No. 4560 |
| 2177b | Seq. ID No. 4561 | Seq. ID No. 4562 |
| 2179b | Seq. ID No. 4565 | Seq. ID No. 4566 |
| 2180 | Seq. ID No. 4567 | Seq. ID No. 4568 |
| 2181b | Seq. ID No. 4571 | Seq. ID No. 4572 |
| 2182 | Seq. ID No. 4573 | Seq. ID No. 4574 |
| 2183 | Seq. ID No. 4575 | Seq. ID No. 4576 |
| 2185 | Seq. ID No. 4577 | Seq. ID No. 4578 |
| 2186 | Seq. ID No. 4581 | Seq. ID No. 4582 |
| 2189b | Seq. ID No. 4585 | Seq. ID No. 4586 |
| 2190b | Seq. ID No. 4589 | Seq. ID No. 4590 |
| 2193 | Seq. ID No. 4591 | Seq. ID No. 4592 |
| 2196 | Seq. ID No. 4597 | Seq. ID No. 4598 |
| 2197a | Seq. ID No. 4599 | Seq. ID No. 4600 |
| 2197b | Seq. ID No. 4601 | Seq. ID No. 4602 |
| 2197c | Seq. ID No. 4603 | Seq. ID No. 4604 |
| 2201a | Seq. ID No. 4605 | Seq. ID No. 4606 |
| 2201b | Seq. ID No. 4607 | Seq. ID No. 4608 |
| 2203 | Seq. ID No. 4609 | Seq. ID No. 4610 |
| 2204 | Seq. ID No. 4611 | Seq. ID No. 4612 |
| 2206a | Seq. ID No. 4613 | Seq. ID No. 4614 |
| 2206b | Seq. ID No. 4615 | Seq. ID No. 4616 |
| 2208a | Seq. ID No. 4617 | Seq. ID No. 4618 |
| 2208b | Seq. ID No. 4619 | Seq. ID No. 4620 |
| 2208c | Seq. ID No. 4621 | Seq. ID No. 4622 |
| 2209 | Seq. ID No. 4623 | Seq. ID No. 4624 |
| 2212b | Seq. ID No. 4627 | Seq. ID No. 4628 |
| 2215b | Seq. ID No. 4631 | Seq. ID No. 4632 |
| 2218 | Seq. ID No. 4637 | Seq. ID No. 4638 |
| 2219b | Seq. ID No. 4641 | Seq. ID No. 4642 |
| 2219c | Seq. ID No. 4643 | Seq. ID No. 4644 |
| 2220a | Seq. ID No. 4645 | Seq. ID No. 4646 |
| 2220b | Seq. ID No. 4647 | Seq. ID No. 4648 |
| 2220c | Seq. ID No. 4649 | Seq. ID No. 4650 |
| 2222 | Seq. ID No. 4653 | Seq. ID No. 4654 |
| 2225 | Seq. ID No. 4659 | Seq. ID No. 4660 |
| 2226 | Seq. ID No. 4661 | Seq. ID No. 4662 |
| 2227 | Seq. ID No. 4663 | Seq. ID No. 4664 |
| 2228a | Seq. ID No. 4665 | Seq. ID No. 4666 |
| 2228b | Seq. ID No. 4667 | Seq. ID No. 4668 |
| 2228c | Seq. ID No. 4669 | Seq. ID No. 4670 |
| 2231 | Seq. ID No. 4671 | Seq. ID No. 4672 |
| 2232 | Seq. ID No. 4673 | Seq. ID No. 4674 |
| 2234a | Seq. ID No. 4675 | Seq. ID No. 4676 |
| 2234b | Seq. ID No. 4677 | Seq. ID No. 4678 |
| 2234c | Seq. ID No. 4679 | Seq. ID No. 4680 |
| 2236b | Seq. ID No. 4683 | Seq. ID No. 4684 |
| 2238 | Seq. ID No. 4685 | Seq. ID No. 4686 |
| 2239b | Seq. ID No. 4687 | Seq. ID No. 4688 |
| 2240b | Seq. ID No. 4691 | Seq. ID No. 4692 |
| 2240c | Seq. ID No. 4693 | Seq. ID No. 4694 |
| 2243a | Seq. ID No. 4695 | Seq. ID No. 4696 |
| 2245 | Seq. ID No. 4699 | Seq. ID No. 4700 |
| 2246 | Seq. ID No. 4701 | Seq. ID No. 4702 |
| 2248 | Seq. ID No. 4703 | Seq. ID No. 4704 |
| 2249 | Seq. ID No. 4705 | Seq. ID No. 4706 |
| 2253d | Seq. ID No. 4713 | Seq. ID No. 4714 |
| 2255b | Seq. ID No. 4717 | Seq. ID No. 4718 |
| 2256 | Seq. ID No. 4719 | Seq. ID No. 4720 |
| 2257 | Seq. ID No. 4721 | Seq. ID No. 4722 |
| 2260b | Seq. ID No. 4727 | Seq. ID No. 4728 |
| 2260c | Seq. ID No. 4729 | Seq. ID No. 4730 |
| 2263 | Seq. ID No. 4733 | Seq. ID No. 4734 |
| 2264a | Seq. ID No. 4735 | Seq. ID No. 4736 |
| 2264b | Seq. ID No. 4737 | Seq. ID No. 4738 |
| 2267 | Seq. ID No. 4739 | Seq. ID No. 4740 |
| 2268 | Seq. ID No. 4741 | Seq. ID No. 4742 |
| 2269 | Seq. ID No. 4743 | Seq. ID No. 4744 |
| 2272 | Seq. ID No. 4747 | Seq. ID No. 4748 |
| 2273 | Seq. ID No. 4749 | Seq. ID No. 4750 |
| 2274 | Seq. ID No. 4751 | Seq. ID No. 4752 |
| 2275b | Seq. ID No. 4755 | Seq. ID No. 4756 |
| 2275c | Seq. ID No. 4757 | Seq. ID No. 4758 |
| 2276c | Seq. ID No. 4763 | Seq. ID No. 4764 |
| 2276d | Seq. ID No. 4765 | Seq. ID No. 4766 |
| 2277a | Seq. ID No. 4767 | Seq. ID No. 4768 |
| 2277b | Seq. ID No. 4769 | Seq. ID No. 4770 |
| 2278b | Seq. ID No. 4773 | Seq. ID No. 4774 |
| 2278c | Seq. ID No. 4775 | Seq. ID No. 4776 |
| 2278d | Seq. ID No. 4777 | Seq. ID No. 4778 |
| 2280 | Seq. ID No. 4779 | Seq. ID No. 4780 |
| 2284 | Seq. ID No. 4781 | Seq. ID No. 4782 |
| 2286b | Seq. ID No. 4787 | Seq. ID No. 4788 |
| 2288 | Seq. ID No. 4789 | Seq. ID No. 4790 |
| 2290 | Seq. ID No. 4791 | Seq. ID No. 4792 |
| 2291a | Seq. ID No. 4793 | Seq. ID No. 4794 |
| 2291b | Seq. ID No. 4795 | Seq. ID No. 4796 |
| 2291c | Seq. ID No. 4797 | Seq. ID No. 4798 |
| 2292b | Seq. ID No. 4801 | Seq. ID No. 4802 |
| 2292c | Seq. ID No. 4803 | Seq. ID No. 4804 |
| 2294 | Seq. ID No. 4805 | Seq. ID No. 4806 |
| 2298 | Seq. ID No. 4809 | Seq. ID No. 4810 |
| 2299 | Seq. ID No. 4811 | Seq. ID No. 4812 |
| 2300 | Seq. ID No. 4813 | Seq. ID No. 4814 |
| 2301 | Seq. ID No. 4815 | Seq. ID No. 4816 |
| 2302 | Seq. ID No. 4817 | Seq. ID No. 4818 |
| 2303 | Seq. ID No. 4819 | Seq. ID No. 4820 |
| 2307 | Seq. ID No. 4825 | Seq. ID No. 4826 |
| 2308 | Seq. ID No. 4827 | Seq. ID No. 4828 |
| 2309a | Seq. ID No. 4829 | Seq. ID No. 4830 |
| 2309b | Seq. ID No. 4831 | Seq. ID No. 4832 |
| 2309c | Seq. ID No. 4833 | Seq. ID No. 4834 |
| 2311 | Seq. ID No. 4837 | Seq. ID No. 4838 |
| 2312a | Seq. ID No. 4839 | Seq. ID No. 4840 |
| 2312b | Seq. ID No. 4841 | Seq. ID No. 4842 |
| 2312c | Seq. ID No. 4843 | Seq. ID No. 4844 |
| 2314 | Seq. ID No. 4845 | Seq. ID No. 4846 |
| 2316 | Seq. ID No. 4849 | Seq. ID No. 4850 |
| 2317 | Seq. ID No. 4851 | Seq. ID No. 4852 |
| 2320 | Seq. ID No. 4853 | Seq. ID No. 4854 |
| 2321a | Seq. ID No. 4855 | Seq. ID No. 4856 |
| 2322 | Seq. ID No. 4861 | Seq. ID No. 4862 |
| 2324 | Seq. ID No. 4863 | Seq. ID No. 4864 |
| 2328 | Seq. ID No. 4867 | Seq. ID No. 4868 |
| 2329d | Seq. ID No. 4875 | Seq. ID No. 4876 |
| 2330 | Seq. ID No. 4877 | Seq. ID No. 4878 |
| 2331 | Seq. ID No. 4879 | Seq. ID No. 4880 |
| 2333 | Seq. ID No. 4881 | Seq. ID No. 4882 |
| 2335 | Seq. ID No. 4883 | Seq. ID No. 4884 |
| 2338a | Seq. ID No. 4889 | Seq. ID No. 4890 |
| 2338b | Seq. ID No. 4891 | Seq. ID No. 4892 |
| 2338c | Seq. ID No. 4893 | Seq. ID No. 4894 |
| 2342a | Seq. ID No. 4895 | Seq. ID No. 4896 |
| 2342b | Seq. ID No. 4897 | Seq. ID No. 4898 |
| 2342c | Seq. ID No. 4899 | Seq. ID No. 4900 |
| 2344 | Seq. ID No. 4901 | Seq. ID No. 4902 |
| 2345 | Seq. ID No. 4903 | Seq. ID No. 4904 |
| 2347 | Seq. ID No. 4905 | Seq. ID No. 4906 |
| 2348 | Seq. ID No. 4907 | Seq. ID No. 4908 |
| 2349a | Seq. ID No. 4909 | Seq. ID No. 4910 |
| 2349b | Seq. ID No. 4911 | Seq. ID No. 4912 |
| 2350a | Seq. ID No. 4913 | Seq. ID No. 4914 |
| 2353 | Seq. ID No. 4915 | Seq. ID No. 4916 |
| 2354 | Seq. ID No. 4917 | Seq. ID No. 4918 |
| 2357 | Seq. ID No. 4919 | Seq. ID No. 4920 |
| 2358 | Seq. ID No. 4921 | Seq. ID No. 4922 |
| 2360 | Seq. ID No. 4923 | Seq. ID No. 4924 |
| 2361b | Seq. ID No. 4927 | Seq. ID No. 4928 |
| 2363b | Seq. ID No. 4931 | Seq. ID No. 4932 |
| 2365b | Seq. ID No. 4935 | Seq. ID No. 4936 |
| 2366a | Seq. ID No. 4937 | Seq. ID No. 4938 |
| 2366b | Seq. ID No. 4939 | Seq. ID No. 4940 |
| 2367 | Seq. ID No. 4941 | Seq. ID No. 4942 |
| 2368 | Seq. ID No. 4943 | Seq. ID No. 4944 |
| 2369 | Seq. ID No. 4945 | Seq. ID No. 4946 |
| 2370 | Seq. ID No. 4947 | Seq. ID No. 4948 |
| 2371a | Seq. ID No. 4949 | Seq. ID No. 4950 |
| 2372 | Seq. ID No. 4951 | Seq. ID No. 4952 |
| 2373 | Seq. ID No. 4953 | Seq. ID No. 4954 |
| 2374b | Seq. ID No. 4957 | Seq. ID No. 4958 |
| 2376 | Seq. ID No. 4959 | Seq. ID No. 4960 |
| 2377a | Seq. ID No. 4961 | Seq. ID No. 4962 |
| 2377b | Seq. ID No. 4963 | Seq. ID No. 4964 |
| 2381 | Seq. ID No. 4973 | Seq. ID No. 4974 |
| 2382c | Seq. ID No. 4975 | Seq. ID No. 4976 |
| 2384a | Seq. ID No. 4977 | Seq. ID No. 4978 |
| 2384b | Seq. ID No. 4979 | Seq. ID No. 4980 |
| 2386b | Seq. ID No. 4981 | Seq. ID No. 4982 |
| 2386c | Seq. ID No. 4983 | Seq. ID No. 4984 |
| 2387 | Seq. ID No. 4985 | Seq. ID No. 4986 |
| 2390 | Seq. ID No. 4987 | Seq. ID No. 4988 |
| 2391 | Seq. ID No. 4989 | Seq. ID No. 4990 |
| 2392 | Seq. ID No. 4991 | Seq. ID No. 4992 |
| 2394 | Seq. ID No. 4993 | Seq. ID No. 4994 |
| 2395 | Seq. ID No. 4995 | Seq. ID No. 4996 |
| 2396 | Seq. ID No. 4997 | Seq. ID No. 4998 |
| 2397 | Seq. ID No. 4999 | Seq. ID No. 5000 |
| 2399a | Seq. ID No. 5003 | Seq. ID No. 5004 |
| 2399b | Seq. ID No. 5005 | Seq. ID No. 5006 |
| 2399c | Seq. ID No. 5007 | Seq. ID No. 5008 |
| 2401 | Seq. ID No. 5009 | Seq. ID No. 5010 |
| 2402a | Seq. ID No. 5011 | Seq. ID No. 5012 |
| 2402b | Seq. ID No. 5013 | Seq. ID No. 5014 |
| 2403b | Seq. ID No. 5017 | Seq. ID No. 5018 |
| 2404a | Seq. ID No. 5019 | Seq. ID No. 5020 |
| 2404b | Seq. ID No. 5021 | Seq. ID No. 5022 |
| 2407a | Seq. ID No. 5023 | Seq. ID No. 5024 |
| 2409a | Seq. ID No. 5025 | Seq. ID No. 5026 |
| 2410 | Seq. ID No. 5029 | Seq. ID No. 5030 |
| 2412 | Seq. ID No. 5031 | Seq. ID No. 5032 |
| 2413a | Seq. ID No. 5033 | Seq. ID No. 5034 |
| 2413b | Seq. ID No. 5035 | Seq. ID No. 5036 |
| 2416b | Seq. ID No. 5041 | Seq. ID No. 5042 |
| 2416c | Seq. ID No. 5043 | Seq. ID No. 5044 |
| 2417a | Seq. ID No. 5045 | Seq. ID No. 5046 |
| 2417b | Seq. ID No. 5047 | Seq. ID No. 5048 |
| 2422b | Seq. ID No. 5057 | Seq. ID No. 5058 |
| 2422c | Seq. ID No. 5059 | Seq. ID No. 5060 |
| 2423b | Seq. ID No. 5063 | Seq. ID No. 5064 |
| 2424a | Seq. ID No. 5065 | Seq. ID No. 5066 |
| 2424b | Seq. ID No. 5067 | Seq. ID No. 5068 |
| 2424c | Seq. ID No. 5069 | Seq. ID No. 5070 |
| 2428b | Seq. ID No. 5075 | Seq. ID No. 5076 |
| 2428c | Seq. ID No. 5077 | Seq. ID No. 5078 |
| 2429 | Seq. ID No. 5079 | Seq. ID No. 5080 |
| 2432 | Seq. ID No. 5081 | Seq. ID No. 5082 |
| 2433 | Seq. ID No. 5083 | Seq. ID No. 5084 |
| 2434 | Seq. ID No. 5085 | Seq. ID No. 5086 |
| 2440 | Seq. ID No. 5087 | Seq. ID No. 5088 |
| 2442b | Seq. ID No. 5091 | Seq. ID No. 5092 |
| 2444c | Seq. ID No. 5099 | Seq. ID No. 5100 |
| 2444d | Seq. ID No. 5101 | Seq. ID No. 5102 |
| 2445 | Seq. ID No. 5103 | Seq. ID No. 5104 |
| 2449b | Seq. ID No. 5109 | Seq. ID No. 5110 |
| 2451 | Seq. ID No. 5111 | Seq. ID No. 5112 |
| 2452 | Seq. ID No. 5113 | Seq. ID No. 5114 |
| 2453 | Seq. ID No. 5115 | Seq. ID No. 5116 |
| 2455 | Seq. ID No. 5117 | Seq. ID No. 5118 |
| 2456b | Seq. ID No. 5121 | Seq. ID No. 5122 |
| 2456d | Seq. ID No. 5125 | Seq. ID No. 5126 |
| 2456e | Seq. ID No. 5127 | Seq. ID No. 5128 |
| 2458c | Seq. ID No. 5133 | Seq. ID No. 5134 |
| 2458f | Seq. ID No. 5139 | Seq. ID No. 5140 |
| 2458g | Seq. ID No. 5141 | Seq. ID No. 5142 |
| 2461b | Seq. ID No. 5145 | Seq. ID No. 5146 |
| 2462a | Seq. ID No. 5147 | Seq. ID No. 5148 |
| 2462b | Seq. ID No. 5149 | Seq. ID No. 5150 |
| 2463a | Seq. ID No. 5151 | Seq. ID No. 5152 |
| 2465a | Seq. ID No. 5155 | Seq. ID No. 5156 |
| 2465b | Seq. ID No. 5157 | Seq. ID No. 5158 |
| 2468a | Seq. ID No. 5159 | Seq. ID No. 5160 |
| 2468b | Seq. ID No. 5161 | Seq. ID No. 5162 |
| 2469b | Seq. ID No. 5165 | Seq. ID No. 5166 |
| 2470 | Seq. ID No. 5167 | Seq. ID No. 5168 |
| 2471 | Seq. ID No. 5169 | Seq. ID No. 5170 |
| 2474c | Seq. ID No. 5177 | Seq. ID No. 5178 |
| 2475b | Seq. ID No. 5181 | Seq. ID No. 5182 |
| 2475c | Seq. ID No. 5183 | Seq. ID No. 5184 |
| 2477 | Seq. ID No. 5185 | Seq. ID No. 5186 |
| 2480 | Seq. ID No. 5187 | Seq. ID No. 5188 |
| 2481a | Seq. ID No. 5189 | Seq. ID No. 5190 |
| 2481b | Seq. ID No. 5191 | Seq. ID No. 5192 |
| 2482 | Seq. ID No. 5193 | Seq. ID No. 5194 |
| 2483 | Seq. ID No. 5195 | Seq. ID No. 5196 |
| 2484a | Seq. ID No. 5197 | Seq. ID No. 5198 |
| 2484b | Seq. ID No. 5199 | Seq. ID No. 5200 |
| 2485 | Seq. ID No. 5201 | Seq. ID No. 5202 |
| 2489 | Seq. ID No. 5203 | Seq. ID No. 5204 |
| 2490 | Seq. ID No. 5205 | Seq. ID No. 5206 |
| 2492b | Seq. ID No. 5209 | Seq. ID No. 5210 |
| 2494 | Seq. ID No. 5211 | Seq. ID No. 5212 |
| 2495b | Seq. ID No. 5215 | Seq. ID No. 5216 |
| 2497b | Seq. ID No. 5219 | Seq. ID No. 5220 |
| 2498b | Seq. ID No. 5221 | Seq. ID No. 5222 |
| 2501a | Seq. ID No. 5229 | Seq. ID No. 5230 |
| 2501b | Seq. ID No. 5231 | Seq. ID No. 5232 |
| 2506 | Seq. ID No. 5239 | Seq. ID No. 5240 |
| 2507b | Seq. ID No. 5241 | Seq. ID No. 5242 |
| 2507c | Seq. ID No. 5243 | Seq. ID No. 5244 |
| 2510 | Seq. ID No. 5245 | Seq. ID No. 5246 |
| 2513b | Seq. ID No. 5251 | Seq. ID No. 5252 |
| 2513c | Seq. ID No. 5253 | Seq. ID No. 5254 |
| 2514 | Seq. ID No. 5255 | Seq. ID No. 5256 |
| 2516b | Seq. ID No. 5261 | Seq. ID No. 5262 |
| 2517a | Seq. ID No. 5263 | Seq. ID No. 5264 |
| 2517b | Seq. ID No. 5265 | Seq. ID No. 5266 |
| 2518a | Seq. ID No. 5267 | Seq. ID No. 5268 |
| 2520c | Seq. ID No. 5277 | Seq. ID No. 5278 |
| 2520d | Seq. ID No. 5279 | Seq. ID No. 5280 |
| 2521 | Seq. ID No. 5281 | Seq. ID No. 5282 |
| 2525a | Seq. ID No. 5285 | Seq. ID No. 5286 |
| 2525b | Seq. ID No. 5287 | Seq. ID No. 5288 |
| 2527b | Seq. ID No. 5291 | Seq. ID No. 5292 |
| 2528b | Seq. ID No. 5295 | Seq. ID No. 5296 |
| 2528c | Seq. ID No. 5297 | Seq. ID No. 5298 |
| 2528d | Seq. ID No. 5299 | Seq. ID No. 5300 |
| 2532b | Seq. ID No. 5301 | Seq. ID No. 5302 |
| 2532c | Seq. ID No. 5303 | Seq. ID No. 5304 |
| 2534 | Seq. ID No. 5305 | Seq. ID No. 5306 |
| 2535a | Seq. ID No. 5307 | Seq. ID No. 5308 |
| 2535b | Seq. ID No. 5309 | Seq. ID No. 5310 |
| 2538 | Seq. ID No. 5311 | Seq. ID No. 5312 |
| 2541 | Seq. ID No. 5313 | Seq. ID No. 5314 |
| 2542b | Seq. ID No. 5317 | Seq. ID No. 5318 |
| 2542c | Seq. ID No. 5319 | Seq. ID No. 5320 |
| 2543 | Seq. ID No. 5321 | Seq. ID No. 5322 |
| 2544 | Seq. ID No. 5323 | Seq. ID No. 5324 |
| 2546 | Seq. ID No. 5327 | Seq. ID No. 5328 |
| 2547b | Seq. ID No. 5329 | Seq. ID No. 5330 |
| 2547c | Seq. ID No. 5331 | Seq. ID No. 5332 |
| 2548a | Seq. ID No. 5333 | Seq. ID No. 5334 |
| 2548b | Seq. ID No. 5335 | Seq. ID No. 5336 |
| 2549 | Seq. ID No. 5337 | Seq. ID No. 5338 |
| 2550e | Seq. ID No. 5343 | Seq. ID No. 5344 |
| 2552 | Seq. ID No. 5345 | Seq. ID No. 5346 |
| 2554a | Seq. ID No. 5349 | Seq. ID No. 5350 |
| 2554b | Seq. ID No. 5351 | Seq. ID No. 5352 |
| 2555c | Seq. ID No. 5357 | Seq. ID No. 5358 |
| 2555d | Seq. ID No. 5359 | Seq. ID No. 5360 |
| 2559 | Seq. ID No. 5365 | Seq. ID No. 5366 |
| 2561 | Seq. ID No. 5367 | Seq. ID No. 5368 |
| 2563 | Seq. ID No. 5373 | Seq. ID No. 5374 |
| 2564b | Seq. ID No. 5377 | Seq. ID No. 5378 |
| 2564c | Seq. ID No. 5379 | Seq. ID No. 5380 |
| 2564d | Seq. ID No. 5381 | Seq. ID No. 5382 |
| 2566 | Seq. ID No. 5383 | Seq. ID No. 5384 |
| 2567b | Seq. ID No. 5387 | Seq. ID No. 5388 |
| 2568 | Seq. ID No. 5389 | Seq. ID No. 5390 |
| 2572 | Seq. ID No. 5391 | Seq. ID No. 5392 |
| 2573 | Seq. ID No. 5393 | Seq. ID No. 5394 |
| 2576 | Seq. ID No. 5397 | Seq. ID No. 5398 |
| 2578 | Seq. ID No. 5399 | Seq. ID No. 5400 |
| 2579b | Seq. ID No. 5401 | Seq. ID No. 5402 |
| 2581 | Seq. ID No. 5403 | Seq. ID No. 5404 |
| 2582a | Seq. ID No. 5405 | Seq. ID No. 5406 |
| 2582b | Seq. ID No. 5407 | Seq. ID No. 5408 |
| 2582c | Seq. ID No. 5409 | Seq. ID No. 5410 |
| 2585 | Seq. ID No. 5411 | Seq. ID No. 5412 |
| 2587b | Seq. ID No. 5415 | Seq. ID No. 5416 |
| 2588 | Seq. ID No. 5417 | Seq. ID No. 5418 |
| 2589 | Seq. ID No. 5419 | Seq. ID No. 5420 |
| 2591 | Seq. ID No. 5421 | Seq. ID No. 5422 |
| 2592 | Seq. ID No. 5423 | Seq. ID No. 5424 |
| 2593a | Seq. ID No. 5425 | Seq. ID No. 5426 |
| 2593b | Seq. ID No. 5427 | Seq. ID No. 5428 |
| 2594b | Seq. ID No. 5431 | Seq. ID No. 5432 |
| 2596 | Seq. ID No. 5433 | Seq. ID No. 5434 |
| 2597a | Seq. ID No. 5435 | Seq. ID No. 5436 |
| 2597b | Seq. ID No. 5437 | Seq. ID No. 5438 |
| 2597c | Seq. ID No. 5439 | Seq. ID No. 5440 |
| 2598 | Seq. ID No. 5441 | Seq. ID No. 5442 |
| 2602 | Seq. ID No. 5443 | Seq. ID No. 5444 |
| 2605b | Seq. ID No. 5447 | Seq. ID No. 5448 |
| 2606 | Seq. ID No. 5449 | Seq. ID No. 5450 |
| 2608 | Seq. ID No. 5451 | Seq. ID No. 5452 |
| 2610 | Seq. ID No. 5453 | Seq. ID No. 5454 |
| 2611 | Seq. ID No. 5455 | Seq. ID No. 5456 |
| 2613 | Seq. ID No. 5457 | Seq. ID No. 5458 |
| 2616e | Seq. ID No. 5467 | Seq. ID No. 5468 |
| 2616g | Seq. ID No. 5471 | Seq. ID No. 5472 |
| 2616h | Seq. ID No. 5473 | Seq. ID No. 5474 |
| 2617b | Seq. ID No. 5477 | Seq. ID No. 5478 |
| 2618a | Seq. ID No. 5479 | Seq. ID No. 5480 |
| 2618b | Seq. ID No. 5481 | Seq. ID No. 5482 |
| 2623b | Seq. ID No. 5491 | Seq. ID No. 5492 |
| 2624a | Seq. ID No. 5493 | Seq. ID No. 5494 |
| 2624b | Seq. ID No. 5495 | Seq. ID No. 5496 |
| 2625b | Seq. ID No. 5499 | Seq. ID No. 5500 |
| 2626b | Seq. ID No. 5503 | Seq. ID No. 5504 |
| 2628 | Seq. ID No. 5509 | Seq. ID No. 5510 |
| 2629b | Seq. ID No. 5513 | Seq. ID No. 5514 |
| 2631 | Seq. ID No. 5515 | Seq. ID No. 5516 |
| 2632 | Seq. ID No. 5517 | Seq. ID No. 5518 |
| 2633a | Seq. ID No. 5519 | Seq. ID No. 5520 |
| 2633b | Seq. ID No. 5521 | Seq. ID No. 5522 |
| 2633e | Seq. ID No. 5527 | Seq. ID No. 5528 |
| 2637a | Seq. ID No. 5529 | Seq. ID No. 5530 |
| 2637b | Seq. ID No. 5531 | Seq. ID No. 5532 |
| 2642 | Seq. ID No. 5533 | Seq. ID No. 5534 |
| 2643b | Seq. ID No. 5537 | Seq. ID No. 5538 |
| 2645 | Seq. ID No. 5539 | Seq. ID No. 5540 |
| 2647 | Seq. ID No. 5541 | Seq. ID No. 5542 |
| 2648a | Seq. ID No. 5543 | Seq. ID No. 5544 |
| 2648b | Seq. ID No. 5545 | Seq. ID No. 5546 |
| 2649 | Seq. ID No. 5547 | Seq. ID No. 5548 |
| 2650 | Seq. ID No. 5549 | Seq. ID No. 5550 |
| 2652a | Seq. ID No. 5551 | Seq. ID No. 5552 |
| 2652b | Seq. ID No. 5553 | Seq. ID No. 5554 |
| 2655b | Seq. ID No. 5557 | Seq. ID No. 5558 |
| 2656 | Seq. ID No. 5559 | Seq. ID No. 5560 |
| 2658b | Seq. ID No. 5563 | Seq. ID No. 5564 |
| 2659b | Seq. ID No. 5567 | Seq. ID No. 5568 |
| 2660 | Seq. ID No. 5569 | Seq. ID No. 5570 |
| 2664b | Seq. ID No. 5575 | Seq. ID No. 5576 |
| 2665 | Seq. ID No. 5577 | Seq. ID No. 5578 |
| 2666b | Seq. ID No. 5581 | Seq. ID No. 5582 |
| 2667a | Seq. ID No. 5583 | Seq. ID No. 5584 |
| 2667b | Seq. ID No. 5585 | Seq. ID No. 5586 |
| 2669b | Seq. ID No. 5589 | Seq. ID No. 5590 |
| 2669c | Seq. ID No. 5591 | Seq. ID No. 5592 |
| 2672 | Seq. ID No. 5593 | Seq. ID No. 5594 |
| 2673 | Seq. ID No. 5595 | Seq. ID No. 5596 |
| 2674b | Seq. ID No. 5599 | Seq. ID No. 5600 |
| 2676 | Seq. ID No. 5601 | Seq. ID No. 5602 |
| 2677c | Seq. ID No. 5607 | Seq. ID No. 5608 |
| 2679c | Seq. ID No. 5613 | Seq. ID No. 5614 |
| 2679d | Seq. ID No. 5615 | Seq. ID No. 5616 |
| 2680a | Seq. ID No. 5617 | Seq. ID No. 5618 |
| 2680b | Seq. ID No. 5619 | Seq. ID No. 5620 |
| 2680c | Seq. ID No. 5621 | Seq. ID No. 5622 |
| 2683b | Seq. ID No. 5625 | Seq. ID No. 5626 |
| 2684a | Seq. ID No. 5627 | Seq. ID No. 5628 |
| 2684b | Seq. ID No. 5629 | Seq. ID No. 5630 |
| 2686 | Seq. ID No. 5631 | Seq. ID No. 5632 |
| 2688c | Seq. ID No. 5637 | Seq. ID No. 5638 |
| 2689a | Seq. ID No. 5639 | Seq. ID No. 5640 |
| 2689b | Seq. ID No. 5641 | Seq. ID No. 5642 |
| 2690b | Seq. ID No. 5645 | Seq. ID No. 5646 |
| 2690c | Seq. ID No. 5647 | Seq. ID No. 5648 |
| 2692b | Seq. ID No. 5649 | Seq. ID No. 5650 |
| 2693 | Seq. ID No. 5651 | Seq. ID No. 5652 |
| 2694b | Seq. ID No. 5655 | Seq. ID No. 5656 |
| 2695 | Seq. ID No. 5657 | Seq. ID No. 5658 |
| 2696b | Seq. ID No. 5661 | Seq. ID No. 5662 |
| 2709 | Seq. ID No. 5665 | Seq. ID No. 5666 |
| 2710c | Seq. ID No. 5667 | Seq. ID No. 5668 |
| 2712 | Seq. ID No. 5669 | Seq. ID No. 5670 |
| 2716b | Seq. ID No. 5677 | Seq. ID No. 5678 |
| 2716c | Seq. ID No. 5679 | Seq. ID No. 5680 |
| 2718b | Seq. ID No. 5683 | Seq. ID No. 5684 |
| 2718c | Seq. ID No. 5685 | Seq. ID No. 5686 |
| 2719a | Seq. ID No. 5687 | Seq. ID No. 5688 |
| 2719b | Seq. ID No. 5689 | Seq. ID No. 5690 |
| 2721a | Seq. ID No. 5691 | Seq. ID No. 5692 |
| 2721b | Seq. ID No. 5693 | Seq. ID No. 5694 |
| 2722b | Seq. ID No. 5697 | Seq. ID No. 5698 |
| 2723 | Seq. ID No. 5699 | Seq. ID No. 5700 |
| 2724 | Seq. ID No. 5701 | Seq. ID No. 5702 |
| 2725a | Seq. ID No. 5703 | Seq. ID No. 5704 |
| 2725b | Seq. ID No. 5705 | Seq. ID No. 5706 |
| 2726 | Seq. ID No. 5707 | Seq. ID No. 5708 |
| 2728b | Seq. ID No. 5711 | Seq. ID No. 5712 |
| 2731a | Seq. ID No. 5713 | Seq. ID No. 5714 |
| 2731b | Seq. ID No. 5715 | Seq. ID No. 5716 |
| 2732 | Seq. ID No. 5717 | Seq. ID No. 5718 |
| 2734a | Seq. ID No. 5719 | Seq. ID No. 5720 |
| 2734b | Seq. ID No. 5721 | Seq. ID No. 5722 |
| 2734c | Seq. ID No. 5723 | Seq. ID No. 5724 |
| 2735a | Seq. ID No. 5725 | Seq. ID No. 5726 |
| 2735b | Seq. ID No. 5727 | Seq. ID No. 5728 |
| 2738d | Seq. ID No. 5735 | Seq. ID No. 5736 |
| 2742b | Seq. ID No. 5739 | Seq. ID No. 5740 |
| 2743a | Seq. ID No. 5741 | Seq. ID No. 5742 |
| 2743b | Seq. ID No. 5743 | Seq. ID No. 5744 |
| 2744 | Seq. ID No. 5745 | Seq. ID No. 5746 |
| 2745a | Seq. ID No. 5747 | Seq. ID No. 5748 |
| 2745b | Seq. ID No. 5749 | Seq. ID No. 5750 |
| 2746 | Seq. ID No. 5751 | Seq. ID No. 5752 |
| 2747a | Seq. ID No. 5753 | Seq. ID No. 5754 |
| 2747b | Seq. ID No. 5755 | Seq. ID No. 5756 |
| 2748a | Seq. ID No. 5757 | Seq. ID No. 5758 |
| 2748b | Seq. ID No. 5759 | Seq. ID No. 5760 |
| 2748c | Seq. ID No. 5761 | Seq. ID No. 5762 |
| 2749d | Seq. ID No. 5769 | Seq. ID No. 5770 |
| 2749e | Seq. ID No. 5771 | Seq. ID No. 5772 |
| 2752 | Seq. ID No. 5777 | Seq. ID No. 5778 |
| 2753 | Seq. ID No. 5779 | Seq. ID No. 5780 |
| 2757b | Seq. ID No. 5783 | Seq. ID No. 5784 |
| 2758b | Seq. ID No. 5787 | Seq. ID No. 5788 |
| 2758d | Seq. ID No. 5791 | Seq. ID No. 5792 |
| 2759b | Seq. ID No. 5795 | Seq. ID No. 5796 |
| 2760b | Seq. ID No. 5799 | Seq. ID No. 5800 |
| 2760c | Seq. ID No. 5801 | Seq. ID No. 5802 |
| 2763b | Seq. ID No. 5807 | Seq. ID No. 5808 |
| 2763c | Seq. ID No. 5809 | Seq. ID No. 5810 |
| 2765b | Seq. ID No. 5813 | Seq. ID No. 5814 |
| 2765c | Seq. ID No. 5815 | Seq. ID No. 5816 |
| 2766b | Seq. ID No. 5819 | Seq. ID No. 5820 |
| 2768a | Seq. ID No. 5821 | Seq. ID No. 5822 |
| 2768b | Seq. ID No. 5823 | Seq. ID No. 5824 |
| 2768c | Seq. ID No. 5825 | Seq. ID No. 5826 |
| 2769a | Seq. ID No. 5827 | Seq. ID No. 5828 |
| 2770 | Seq. ID No. 5829 | Seq. ID No. 5830 |
| 2771a | Seq. ID No. 5831 | Seq. ID No. 5832 |
| 2771b | Seq. ID No. 5833 | Seq. ID No. 5834 |
| 2773 | Seq. ID No. 5835 | Seq. ID No. 5836 |
| 2774a | Seq. ID No. 5837 | Seq. ID No. 5838 |
| 2774b | Seq. ID No. 5839 | Seq. ID No. 5840 |
| 2774c | Seq. ID No. 5841 | Seq. ID No. 5842 |
| 2776b | Seq. ID No. 5845 | Seq. ID No. 5846 |
| 2777 | Seq. ID No. 5847 | Seq. ID No. 5848 |
| 2778 | Seq. ID No. 5849 | Seq. ID No. 5850 |
| 2779 | Seq. ID No. 5851 | Seq. ID No. 5852 |
| 2784 | Seq. ID No. 5855 | Seq. ID No. 5856 |
| 2785a | Seq. ID No. 5857 | Seq. ID No. 5858 |
| 2785b | Seq. ID No. 5859 | Seq. ID No. 5860 |
| 2786a | Seq. ID No. 5861 | Seq. ID No. 5862 |
| 2786b | Seq. ID No. 5863 | Seq. ID No. 5864 |
| 2786c | Seq. ID No. 5865 | Seq. ID No. 5866 |
| 2787b | Seq. ID No. 5869 | Seq. ID No. 5870 |
| 2787c | Seq. ID No. 5871 | Seq. ID No. 5872 |
| 2788b | Seq. ID No. 5873 | Seq. ID No. 5874 |
| 2789 | Seq. ID No. 5875 | Seq. ID No. 5876 |
| 2791 | Seq. ID No. 5879 | Seq. ID No. 5880 |
| 2793 | Seq. ID No. 5881 | Seq. ID No. 5882 |
| 2794 | Seq. ID No. 5883 | Seq. ID No. 5884 |
| 2799a | Seq. ID No. 5889 | Seq. ID No. 5890 |
| 2801b | Seq. ID No. 5895 | Seq. ID No. 5896 |
| 2804 | Seq. ID No. 5897 | Seq. ID No. 5898 |
| 2805b | Seq. ID No. 5901 | Seq. ID No. 5902 |
| 2809 | Seq. ID No. 5903 | Seq. ID No. 5904 |
| 2810d | Seq. ID No. 5911 | Seq. ID No. 5912 |
| 2812 | Seq. ID No. 5913 | Seq. ID No. 5914 |
| 2814 | Seq. ID No. 5915 | Seq. ID No. 5916 |
| 2815 | Seq. ID No. 5917 | Seq. ID No. 5918 |
| 2816 | Seq. ID No. 5919 | Seq. ID No. 5920 |
| 2818d | Seq. ID No. 5927 | Seq. ID No. 5928 |
| 2818e | Seq. ID No. 5929 | Seq. ID No. 5930 |
| 2820 | Seq. ID No. 5933 | Seq. ID No. 5934 |
| 2821a | Seq. ID No. 5935 | Seq. ID No. 5936 |
| 2821b | Seq. ID No. 5937 | Seq. ID No. 5938 |
| 2822b | Seq. ID No. 5941 | Seq. ID No. 5942 |
| 2824a | Seq. ID No. 5945 | Seq. ID No. 5946 |
| 2824b | Seq. ID No. 5947 | Seq. ID No. 5948 |
| 2829b | Seq. ID No. 5951 | Seq. ID No. 5952 |
| 2834a | Seq. ID No. 5959 | Seq. ID No. 5960 |
| 2834b | Seq. ID No. 5961 | Seq. ID No. 5962 |
| 2837a | Seq. ID No. 5963 | Seq. ID No. 5964 |
| 2837b | Seq. ID No. 5965 | Seq. ID No. 5966 |
| 2842a | Seq. ID No. 5967 | Seq. ID No. 5968 |
| 2842b | Seq. ID No. 5969 | Seq. ID No. 5970 |
| 2843a | Seq. ID No. 5971 | Seq. ID No. 5972 |
| 2843b | Seq. ID No. 5973 | Seq. ID No. 5974 |
| 2846a | Seq. ID No. 5977 | Seq. ID No. 5978 |
| 2846b | Seq. ID No. 5979 | Seq. ID No. 5980 |
| 2848 | Seq. ID No. 5981 | Seq. ID No. 5982 |
| 2851 | Seq. ID No. 5985 | Seq. ID No. 5986 |
| 2852 | Seq. ID No. 5987 | Seq. ID No. 5988 |
| 2853 | Seq. ID No. 5989 | Seq. ID No. 5990 |
| 2854a | Seq. ID No. 5991 | Seq. ID No. 5992 |
| 2854b | Seq. ID No. 5993 | Seq. ID No. 5994 |
| 2855 | Seq. ID No. 5995 | Seq. ID No. 5996 |
| 2856b | Seq. ID No. 5999 | Seq. ID No. 6000 |
| 2856c | Seq. ID No. 6001 | Seq. ID No. 6002 |
| 2857 | Seq. ID No. 6003 | Seq. ID No. 6004 |
| 2860b | Seq. ID No. 6009 | Seq. ID No. 6010 |
| 2860c | Seq. ID No. 6011 | Seq. ID No. 6012 |
| 2862 | Seq. ID No. 6013 | Seq. ID No. 6014 |
| 2863 | Seq. ID No. 6015 | Seq. ID No. 6016 |
| 2864 | Seq. ID No. 6017 | Seq. ID No. 6018 |
| 2869b | Seq. ID No. 6025 | Seq. ID No. 6026 |
| 2869c | Seq. ID No. 6027 | Seq. ID No. 6028 |
| 2871 | Seq. ID No. 6029 | Seq. ID No. 6030 |
| 2872 | Seq. ID No. 6031 | Seq. ID No. 6032 |
| 2875 | Seq. ID No. 6033 | Seq. ID No. 6034 |
| 2877 | Seq. ID No. 6035 | Seq. ID No. 6036 |
| 2879b | Seq. ID No. 6039 | Seq. ID No. 6040 |
| 2879c | Seq. ID No. 6041 | Seq. ID No. 6042 |
| 2881 | Seq. ID No. 6043 | Seq. ID No. 6044 |
| 2882 | Seq. ID No. 6045 | Seq. ID No. 6046 |
| 2883b | Seq. ID No. 6049 | Seq. ID No. 6050 |
| 2883c | Seq. ID No. 6051 | Seq. ID No. 6052 |
| 2883d | Seq. ID No. 6053 | Seq. ID No. 6054 |
| 2886a | Seq. ID No. 6055 | Seq. ID No. 6056 |
| 2886b | Seq. ID No. 6057 | Seq. ID No. 6058 |
| 2887b | Seq. ID No. 6061 | Seq. ID No. 6062 |
| 2888 | Seq. ID No. 6063 | Seq. ID No. 6064 |
| 2890 | Seq. ID No. 6065 | Seq. ID No. 6066 |
| 2891b | Seq. ID No. 6069 | Seq. ID No. 6070 |
| 2891c | Seq. ID No. 6071 | Seq. ID No. 6072 |
| 2893b | Seq. ID No. 6073 | Seq. ID No. 6074 |
| 2894b | Seq. ID No. 6077 | Seq. ID No. 6078 |
| 2894c | Seq. ID No. 6079 | Seq. ID No. 6080 |
| 2897b | Seq. ID No. 6085 | Seq. ID No. 6086 |
| 2900a | Seq. ID No. 6087 | Seq. ID No. 6088 |
| 2900b | Seq. ID No. 6089 | Seq. ID No. 6090 |
| 2901b | Seq. ID No. 6093 | Seq. ID No. 6094 |
| 2902 | Seq. ID No. 6095 | Seq. ID No. 6096 |
| 2905b | Seq. ID No. 6101 | Seq. ID No. 6102 |
| 2906 | Seq. ID No. 6103 | Seq. ID No. 6104 |
| 2909c | Seq. ID No. 6109 | Seq. ID No. 6110 |
| 2909d | Seq. ID No. 6111 | Seq. ID No. 6112 |
| 2913b | Seq. ID No. 6117 | Seq. ID No. 6118 |
| 2915b | Seq. ID No. 6121 | Seq. ID No. 6122 |
| 2915c | Seq. ID No. 6123 | Seq. ID No. 6124 |
| 2918 | Seq. ID No. 6129 | Seq. ID No. 6130 |
| 2920 | Seq. ID No. 6131 | Seq. ID No. 6132 |
| 2921 | Seq. ID No. 6133 | Seq. ID No. 6134 |
| 2922b | Seq. ID No. 6135 | Seq. ID No. 6136 |
| 2923a | Seq. ID No. 6137 | Seq. ID No. 6138 |
| 2923b | Seq. ID No. 6139 | Seq. ID No. 6140 |
| 2923c | Seq. ID No. 6141 | Seq. ID No. 6142 |
| 2925b | Seq. ID No. 6145 | Seq. ID No. 6146 |
| 2926c | Seq. ID No. 6151 | Seq. ID No. 6152 |
| 2928b | Seq. ID No. 6155 | Seq. ID No. 6156 |
| 2928c | Seq. ID No. 6157 | Seq. ID No. 6158 |
| 2929b | Seq. ID No. 6159 | Seq. ID No. 6160 |
| 2931c | Seq. ID No. 6165 | Seq. ID No. 6166 |
| 2933a | Seq. ID No. 6167 | Seq. ID No. 6168 |
| 2933b | Seq. ID No. 6169 | Seq. ID No. 6170 |
| 2934 | Seq. ID No. 6171 | Seq. ID No. 6172 |
| 2935b | Seq. ID No. 6175 | Seq. ID No. 6176 |
| 2936 | Seq. ID No. 6177 | Seq. ID No. 6178 |
| 2939b | Seq. ID No. 6181 | Seq. ID No. 6182 |
| 2941c | Seq. ID No. 6187 | Seq. ID No. 6188 |
| 2943c | Seq. ID No. 6193 | Seq. ID No. 6194 |
| 2943d | Seq. ID No. 6195 | Seq. ID No. 6196 |
| 2944b | Seq. ID No. 6197 | Seq. ID No. 6198 |
| 2945b | Seq. ID No. 6199 | Seq. ID No. 6200 |
| 2947 | Seq. ID No. 6201 | Seq. ID No. 6202 |
| 2948d | Seq. ID No. 6209 | Seq. ID No. 6210 |
| 2950b | Seq. ID No. 6213 | Seq. ID No. 6214 |
| 2951 | Seq. ID No. 6215 | Seq. ID No. 6216 |
| 2952 | Seq. ID No. 6217 | Seq. ID No. 6218 |
| 2954 | Seq. ID No. 6219 | Seq. ID No. 6220 |
| 2955f | Seq. ID No. 6231 | Seq. ID No. 6232 |
| 2955g | Seq. ID No. 6233 | Seq. ID No. 6234 |
| 2957 | Seq. ID No. 6235 | Seq. ID No. 6236 |
| 2958b | Seq. ID No. 6239 | Seq. ID No. 6240 |
| 2958c | Seq. ID No. 6241 | Seq. ID No. 6242 |
| 2959 | Seq. ID No. 6243 | Seq. ID No. 6244 |
| 2962b | Seq. ID No. 6249 | Seq. ID No. 6250 |
| 2965c | Seq. ID No. 6257 | Seq. ID No. 6258 |
| 2966 | Seq. ID No. 6259 | Seq. ID No. 6260 |
| 2967 | Seq. ID No. 6261 | Seq. ID No. 6262 |
| 2969 | Seq. ID No. 6263 | Seq. ID No. 6264 |
| 2970b | Seq. ID No. 6267 | Seq. ID No. 6268 |
| 2971a | Seq. ID No. 6269 | Seq. ID No. 6270 |
| 2971b | Seq. ID No. 6271 | Seq. ID No. 6272 |
| 2974b | Seq. ID No. 6275 | Seq. ID No. 6276 |
| 2975 | Seq. ID No. 6277 | Seq. ID No. 6278 |
| 2977a | Seq. ID No. 6279 | Seq. ID No. 6280 |
| 2977b | Seq. ID No. 6281 | Seq. ID No. 6282 |
| 2978a | Seq. ID No. 6283 | Seq. ID No. 6284 |
| 2978b | Seq. ID No. 6285 | Seq. ID No. 6286 |
| 2979 | Seq. ID No. 6287 | Seq. ID No. 6288 |
| 2980c | Seq. ID No. 6293 | Seq. ID No. 6294 |
| 2980d | Seq. ID No. 6295 | Seq. ID No. 6296 |
| 2980e | Seq. ID No. 6297 | Seq. ID No. 6298 |
| 2984 | Seq. ID No. 6299 | Seq. ID No. 6300 |
| 2986c | Seq. ID No. 6305 | Seq. ID No. 6306 |
| 2988b | Seq. ID No. 6309 | Seq. ID No. 6310 |
| 2988c | Seq. ID No. 6311 | Seq. ID No. 6312 |
| 2990 | Seq. ID No. 6315 | Seq. ID No. 6316 |
| 2991a | Seq. ID No. 6317 | Seq. ID No. 6318 |
| 2991b | Seq. ID No. 6319 | Seq. ID No. 6320 |
| 2991c | Seq. ID No. 6321 | Seq. ID No. 6322 |
| 2992 | Seq. ID No. 6323 | Seq. ID No. 6324 |
| 2993a | Seq. ID No. 6325 | Seq. ID No. 6326 |
| 2993b | Seq. ID No. 6327 | Seq. ID No. 6328 |
| 2995a | Seq. ID No. 6329 | Seq. ID No. 6330 |
| 2995b | Seq. ID No. 6331 | Seq. ID No. 6332 |
| 2998a | Seq. ID No. 6335 | Seq. ID No. 6336 |
| 2998b | Seq. ID No. 6337 | Seq. ID No. 6338 |
| 3001 | Seq. ID No. 6341 | Seq. ID No. 6342 |
| 3005 | Seq. ID No. 6345 | Seq. ID No. 6346 |
| 3008b | Seq. ID No. 6349 | Seq. ID No. 6350 |
| 3008c | Seq. ID No. 6351 | Seq. ID No. 6352 |
| 3008d | Seq. ID No. 6353 | Seq. ID No. 6354 |
| 3012 | Seq. ID No. 6359 | Seq. ID No. 6360 |
| 3015 | Seq. ID No. 6361 | Seq. ID No. 6362 |
| 3016c | Seq. ID No. 6367 | Seq. ID No. 6368 |
| 3017 | Seq. ID No. 6369 | Seq. ID No. 6370 |
| 3018a | Seq. ID No. 6371 | Seq. ID No. 6372 |
| 3018b | Seq. ID No. 6373 | Seq. ID No. 6374 |
| 3020 | Seq. ID No. 6375 | Seq. ID No. 6376 |
| 3021 | Seq. ID No. 6377 | Seq. ID No. 6378 |
| 3022 | Seq. ID No. 6379 | Seq. ID No. 6380 |
| 3023 | Seq. ID No. 6381 | Seq. ID No. 6382 |
| 3024b | Seq. ID No. 6385 | Seq. ID No. 6386 |
| 3026b | Seq. ID No. 6389 | Seq. ID No. 6390 |
| 3028b | Seq. ID No. 6393 | Seq. ID No. 6394 |
| 3028c | Seq. ID No. 6395 | Seq. ID No. 6396 |
| 3029b | Seq. ID No. 6399 | Seq. ID No. 6400 |
| 3033a | Seq. ID No. 6409 | Seq. ID No. 6410 |
| 3033b | Seq. ID No. 6411 | Seq. ID No. 6412 |
| 3035 | Seq. ID No. 6413 | Seq. ID No. 6414 |
| 3037b | Seq. ID No. 6417 | Seq. ID No. 6418 |
| 3040a | Seq. ID No. 6421 | Seq. ID No. 6422 |
| 3040b | Seq. ID No. 6423 | Seq. ID No. 6424 |
| 3042a | Seq. ID No. 6425 | Seq. ID No. 6426 |
| 3042b | Seq. ID No. 6427 | Seq. ID No. 6428 |
| 3043a | Seq. ID No. 6429 | Seq. ID No. 6430 |
| 3043b | Seq. ID No. 6431 | Seq. ID No. 6432 |
| 3049a | Seq. ID No. 6441 | Seq. ID No. 6442 |
| 3049b | Seq. ID No. 6443 | Seq. ID No. 6444 |
| 3049c | Seq. ID No. 6445 | Seq. ID No. 6446 |
| 3052 | Seq. ID No. 6451 | Seq. ID No. 6452 |
| 3054 | Seq. ID No. 6453 | Seq. ID No. 6454 |
| 3057 | Seq. ID No. 6459 | Seq. ID No. 6460 |
| 3059 | Seq. ID No. 6461 | Seq. ID No. 6462 |
| 3060 | Seq. ID No. 6463 | Seq. ID No. 6464 |
| 3061 | Seq. ID No. 6465 | Seq. ID No. 6466 |
| 3062 | Seq. ID No. 6467 | Seq. ID No. 6468 |
| 3063a | Seq. ID No. 6469 | Seq. ID No. 6470 |
| 3063b | Seq. ID No. 6471 | Seq. ID No. 6472 |
| 3064b | Seq. ID No. 6473 | Seq. ID No. 6474 |
| 3066 | Seq. ID No. 6479 | Seq. ID No. 6480 |
| 3068 | Seq. ID No. 6483 | Seq. ID No. 6484 |
| 3074 | Seq. ID No. 6493 | Seq. ID No. 6494 |
| 3075 | Seq. ID No. 6495 | Seq. ID No. 6496 |
| 3076 | Seq. ID No. 6497 | Seq. ID No. 6498 |
| 3077b | Seq. ID No. 6501 | Seq. ID No. 6502 |
| 3077c | Seq. ID No. 6503 | Seq. ID No. 6504 |
| 3078 | Seq. ID No. 6505 | Seq. ID No. 6506 |
| 3079 | Seq. ID No. 6507 | Seq. ID No. 6508 |
| 3081b | Seq. ID No. 6511 | Seq. ID No. 6512 |
| 3085 | Seq. ID No. 6517 | Seq. ID No. 6518 |
| 3087c | Seq. ID No. 6523 | Seq. ID No. 6524 |
| 3088 | Seq. ID No. 6525 | Seq. ID No. 6526 |
| 3092a | Seq. ID No. 6535 | Seq. ID No. 6536 |
| 3092b | Seq. ID No. 6537 | Seq. ID No. 6538 |
| 3095 | Seq. ID No. 6539 | Seq. ID No. 6540 |
| 3096a | Seq. ID No. 6541 | Seq. ID No. 6542 |
| 3096b | Seq. ID No. 6543 | Seq. ID No. 6544 |
| 3097 | Seq. ID No. 6545 | Seq. ID No. 6546 |
| 3098a | Seq. ID No. 6547 | Seq. ID No. 6548 |
| 3098b | Seq. ID No. 6549 | Seq. ID No. 6550 |
| 3098c | Seq. ID No. 6551 | Seq. ID No. 6552 |
| 3099 | Seq. ID No. 6553 | Seq. ID No. 6554 |
| 3100a | Seq. ID No. 6555 | Seq. ID No. 6556 |
| 3100b | Seq. ID No. 6557 | Seq. ID No. 6558 |
| 3101 | Seq. ID No. 6559 | Seq. ID No. 6560 |
| 3103b | Seq. ID No. 6563 | Seq. ID No. 6564 |
| 3107b | Seq. ID No. 6573 | Seq. ID No. 6574 |
| 3108a | Seq. ID No. 6575 | Seq. ID No. 6576 |
| 3108b | Seq. ID No. 6577 | Seq. ID No. 6578 |
| 3109 | Seq. ID No. 6579 | Seq. ID No. 6580 |
| 3110b | Seq. ID No. 6583 | Seq. ID No. 6584 |
| 3113 | Seq. ID No. 6585 | Seq. ID No. 6586 |
| 3116 | Seq. ID No. 6587 | Seq. ID No. 6588 |
| 3118b | Seq. ID No. 6591 | Seq. ID No. 6592 |
| 3119c | Seq. ID No. 6597 | Seq. ID No. 6598 |
| 3119e | Seq. ID No. 6601 | Seq. ID No. 6602 |
| 3119f | Seq. ID No. 6603 | Seq. ID No. 6604 |
| 3121 | Seq. ID No. 6605 | Seq. ID No. 6606 |
| 3122b | Seq. ID No. 6609 | Seq. ID No. 6610 |
| 3123a | Seq. ID No. 6611 | Seq. ID No. 6612 |
| 3123b | Seq. ID No. 6613 | Seq. ID No. 6614 |
| 3124 | Seq. ID No. 6615 | Seq. ID No. 6616 |
| 3126 | Seq. ID No. 6617 | Seq. ID No. 6618 |
| 3129a | Seq. ID No. 6619 | Seq. ID No. 6620 |
| 3129b | Seq. ID No. 6621 | Seq. ID No. 6622 |
| 3131 | Seq. ID No. 6623 | Seq. ID No. 6624 |
| 3134b | Seq. ID No. 6627 | Seq. ID No. 6628 |
| 3138 | Seq. ID No. 6629 | Seq. ID No. 6630 |
| 3140 | Seq. ID No. 6631 | Seq. ID No. 6632 |
| 3141 | Seq. ID No. 6633 | Seq. ID No. 6634 |
| 3145b | Seq. ID No. 6637 | Seq. ID No. 6638 |
| 3145c | Seq. ID No. 6639 | Seq. ID No. 6640 |
| 3147b | Seq. ID No. 6643 | Seq. ID No. 6644 |
| 3147c | Seq. ID No. 6645 | Seq. ID No. 6646 |
| 3150a | Seq. ID No. 6649 | Seq. ID No. 6650 |

Listed in Table 21, are 615 ORFs detected by another ORF finder program (Signal Search) of Applicant's assignee that searches for transmembrane regions between two Stop codons and a Start codon immediately upstream of the transmembrane region.

TABLE 21
SIGNAL SEARCH ORFS

| ORF Number | DNA SEQ ID Number | Protein SEQ ID Number |
|---|---|---|
| 4a | Seq. ID No. 7 | Seq. ID No. 8 |
| 4b | Seq. ID No. 9 | Seq. ID No. 10 |
| 23a | Seq. ID No. 33 | Seq. ID No. 34 |
| 38b | Seq. ID No. 47 | Seq. ID No. 48 |
| 44b | Seq. ID No. 67 | Seq. ID No. 68 |
| 51a | Seq. ID No. 85 | Seq. ID No. 86 |
| 51b | Seq. ID No. 87 | Seq. ID No. 88 |
| 66a | Seq. ID No. 123 | Seq. ID No. 124 |
| 73b | Seq. ID No. 133 | Seq. ID No. 134 |
| 80a | Seq. ID No. 141 | Seq. ID No. 142 |
| 86 | Seq. ID No. 159 | Seq. ID No. 160 |
| 87b | Seq. ID No. 163 | Seq. ID No. 164 |
| 99 | Seq. ID No. 191 | Seq. ID No. 192 |
| 109a | Seq. ID No. 209 | Seq. ID No. 210 |
| 109b | Seq. ID No. 211 | Seq. ID No. 212 |
| 109d | Seq. ID No. 215 | Seq. ID No. 216 |
| 111a | Seq. ID No. 217 | Seq. ID No. 218 |
| 114 | Seq. ID No. 225 | Seq. ID No. 226 |
| 124a | Seq. ID No. 235 | Seq. ID No. 236 |
| 124b | Seq. ID No. 237 | Seq. ID No. 238 |
| 129a | Seq. ID No. 249 | Seq. ID No. 250 |
| 129b | Seq. ID No. 251 | Seq. ID No. 252 |
| 132a | Seq. ID No. 255 | Seq. ID No. 256 |
| 138a | Seq. ID No. 271 | Seq. ID No. 272 |
| 143 | Seq. ID No. 277 | Seq. ID No. 278 |
| 145a | Seq. ID No. 285 | Seq. ID No. 286 |
| 145b | Seq. ID No. 287 | Seq. ID No. 288 |
| 145c | Seq. ID No. 289 | Seq. ID No. 290 |
| 162a | Seq. ID No. 327 | Seq. ID No. 328 |
| 162b | Seq. ID No. 329 | Seq. ID No. 330 |
| 180a | Seq. ID No. 373 | Seq. ID No. 374 |
| 180b | Seq. ID No. 375 | Seq. ID No. 376 |
| 201a | Seq. ID No. 419 | Seq. ID No. 420 |
| 239a | Seq. ID No. 487 | Seq. ID No. 488 |
| 245a | Seq. ID No. 497 | Seq. ID No. 498 |
| 245b | Seq. ID No. 499 | Seq. ID No. 500 |
| 246a | Seq. ID No. 503 | Seq. ID No. 504 |
| 246b | Seq. ID No. 505 | Seq. ID No. 506 |
| 248a | Seq. ID No. 509 | Seq. ID No. 510 |
| 248b | Seq. ID No. 511 | Seq. ID No. 512 |
| 249b | Seq. ID No. 515 | Seq. ID No. 516 |
| 255a | Seq. ID No. 525 | Seq. ID No. 526 |
| 255b | Seq. ID No. 527 | Seq. ID No. 528 |
| 263a | Seq. ID No. 545 | Seq. ID No. 546 |
| 273a | Seq. ID No. 567 | Seq. ID No. 568 |
| 273b | Seq. ID No. 569 | Seq. ID No. 570 |
| 277c | Seq. ID No. 575 | Seq. ID No. 576 |
| 279a | Seq. ID No. 577 | Seq. ID No. 578 |
| 279b | Seq. ID No. 579 | Seq. ID No. 580 |
| 283b | Seq. ID No. 593 | Seq. ID No. 594 |
| 297 | Seq. ID No. 617 | Seq. ID No. 618 |
| 311 | Seq. ID No. 637 | Seq. ID No. 638 |
| 313a | Seq. ID No. 639 | Seq. ID No. 640 |
| 313b | Seq. ID No. 641 | Seq. ID No. 642 |
| 313d | Seq. ID No. 645 | Seq. ID No. 646 |
| 317a | Seq. ID No. 653 | Seq. ID No. 654 |
| 317b | Seq. ID No. 655 | Seq. ID No. 656 |
| 318b | Seq. ID No. 659 | Seq. ID No. 660 |
| 321 | Seq. ID No. 669 | Seq. ID No. 670 |
| 324 | Seq. ID No. 675 | Seq. ID No. 676 |
| 327a | Seq. ID No. 677 | Seq. ID No. 678 |
| 327b | Seq. ID No. 679 | Seq. ID No. 680 |
| 342a | Seq. ID No. 715 | Seq. ID No. 716 |
| 342b | Seq. ID No. 717 | Seq. ID No. 718 |
| 342c | Seq. ID No. 719 | Seq. ID No. 720 |
| 342d | Seq. ID No. 721 | Seq. ID No. 722 |
| 342e | Seq. ID No. 723 | Seq. ID No. 724 |
| 349c | Seq. ID No. 747 | Seq. ID No. 748 |
| 350b | Seq. ID No. 751 | Seq. ID No. 752 |
| 352a | Seq. ID No. 755 | Seq. ID No. 756 |
| 352b | Seq. ID No. 757 | Seq. ID No. 758 |
| 352c | Seq. ID No. 759 | Seq. ID No. 760 |
| 354a | Seq. ID No. 761 | Seq. ID No. 762 |
| 354b | Seq. ID No. 763 | Seq. ID No. 764 |
| 356a | Seq. ID No. 769 | Seq. ID No. 770 |
| 358a | Seq. ID No. 777 | Seq. ID No. 778 |
| 358b | Seq. ID No. 779 | Seq. ID No. 780 |
| 372d | Seq. ID No. 807 | Seq. ID No. 808 |
| 374 | Seq. ID No. 809 | Seq. ID No. 810 |
| 382b | Seq. ID No. 819 | Seq. ID No. 820 |
| 384a | Seq. ID No. 825 | Seq. ID No. 826 |
| 403b | Seq. ID No. 863 | Seq. ID No. 864 |
| 418a | Seq. ID No. 893 | Seq. ID No. 894 |
| 419c | Seq. ID No. 901 | Seq. ID No. 902 |
| 431 | Seq. ID No. 935 | Seq. ID No. 936 |
| 437c | Seq. ID No. 943 | Seq. ID No. 944 |
| 441b | Seq. ID No. 955 | Seq. ID No. 956 |
| 448 | Seq. ID No. 967 | Seq. ID No. 968 |
| 452b | Seq. ID No. 979 | Seq. ID No. 980 |
| 462 | Seq. ID No. 1005 | Seq. ID No. 1006 |
| 463b | Seq. ID No. 1009 | Seq. ID No. 1010 |
| 464a | Seq. ID No. 1011 | Seq. ID No. 1012 |
| 464b | Seq. ID No. 1013 | Seq. ID No. 1014 |
| 464c | Seq. ID No. 1015 | Seq. ID No. 1016 |
| 465b | Seq. ID No. 1019 | Seq. ID No. 1020 |
| 472a | Seq. ID No. 1029 | Seq. ID No. 1030 |
| 472b | Seq. ID No. 1031 | Seq. ID No. 1032 |
| 472c | Seq. ID No. 1033 | Seq. ID No. 1034 |
| 472e | Seq. ID No. 1037 | Seq. ID No. 1038 |
| 472f | Seq. ID No. 1039 | Seq. ID No. 1040 |
| 476 | Seq. ID No. 1045 | Seq. ID No. 1046 |
| 480a | Seq. ID No. 1055 | Seq. ID No. 1056 |
| 492 | Seq. ID No. 1075 | Seq. ID No. 1076 |
| 507a | Seq. ID No. 1105 | Seq. ID No. 1106 |
| 521b | Seq. ID No. 1131 | Seq. ID No. 1132 |
| 524a | Seq. ID No. 1135 | Seq. ID No. 1136 |
| 524b | Seq. ID No. 1137 | Seq. ID No. 1138 |
| 541a | Seq. ID No. 1165 | Seq. ID No. 1166 |
| 541b | Seq. ID No. 1167 | Seq. ID No. 1168 |
| 541c | Seq. ID No. 1169 | Seq. ID No. 1170 |
| 545a | Seq. ID No. 1183 | Seq. ID No. 1184 |
| 546c | Seq. ID No. 1193 | Seq. ID No. 1194 |
| 547b | Seq. ID No. 1197 | Seq. ID No. 1198 |
| 556 | Seq. ID No. 1213 | Seq. ID No. 1214 |
| 561 | Seq. ID No. 1223 | Seq. ID No. 1224 |
| 564a | Seq. ID No. 1227 | Seq. ID No. 1228 |
| 564b | Seq. ID No. 1229 | Seq. ID No. 1230 |
| 570b | Seq. ID No. 1237 | Seq. ID No. 1238 |
| 576a | Seq. ID No. 1243 | Seq. ID No. 1244 |
| 587a | Seq. ID No. 1265 | Seq. ID No. 1266 |
| 587b | Seq. ID No. 1267 | Seq. ID No. 1268 |
| 587c | Seq. ID No. 1269 | Seq. ID No. 1270 |
| 587d | Seq. ID No. 1271 | Seq. ID No. 1272 |
| 595a | Seq. ID No. 1283 | Seq. ID No. 1284 |
| 596a | Seq. ID No. 1287 | Seq. ID No. 1288 |
| 596c | Seq. ID No. 1291 | Seq. ID No. 1292 |
| 598b | Seq. ID No. 1297 | Seq. ID No. 1298 |
| 600b | Seq. ID No. 1305 | Seq. ID No. 1306 |
| 609a | Seq. ID No. 1315 | Seq. ID No. 1316 |
| 613a | Seq. ID No. 1325 | Seq. ID No. 1326 |
| 613b | Seq. ID No. 1327 | Seq. ID No. 1328 |
| 613c | Seq. ID No. 1329 | Seq. ID No. 1330 |
| 613d | Seq. ID No. 1331 | Seq. ID No. 1332 |
| 619a | Seq. ID No. 1341 | Seq. ID No. 1342 |
| 624c | Seq. ID No. 1353 | Seq. ID No. 1354 |
| 631c | Seq. ID No. 1367 | Seq. ID No. 1368 |
| 645 | Seq. ID No. 1401 | Seq. ID No. 1402 |
| 665 | Seq. ID No. 1429 | Seq. ID No. 1430 |
| 674a | Seq. ID No. 1435 | Seq. ID No. 1436 |
| 679a | Seq. ID No. 1445 | Seq. ID No. 1446 |
| 700a | Seq. ID No. 1481 | Seq. ID No. 1482 |
| 722a | Seq. ID No. 1513 | Seq. ID No. 1514 |
| 722b | Seq. ID No. 1515 | Seq. ID No. 1516 |
| 722c | Seq. ID No. 1517 | Seq. ID No. 1518 |
| 728a | Seq. ID No. 1529 | Seq. ID No. 1530 |
| 730a | Seq. ID No. 1533 | Seq. ID No. 1534 |
| 730b | Seq. ID No. 1535 | Seq. ID No. 1536 |
| 738a | Seq. ID No. 1545 | Seq. ID No. 1546 |
| 738b | Seq. ID No. 1547 | Seq. ID No. 1548 |
| 739 | Seq. ID No. 1551 | Seq. ID No. 1552 |
| 743a | Seq. ID No. 1559 | Seq. ID No. 1560 |
| 744 | Seq. ID No. 1563 | Seq. ID No. 1564 |
| 746b | Seq. ID No. 1571 | Seq. ID No. 1572 |
| 749 | Seq. ID No. 1579 | Seq. ID No. 1580 |
| 754a | Seq. ID No. 1583 | Seq. ID No. 1584 |
| 754b | Seq. ID No. 1585 | Seq. ID No. 1586 |
| 772a | Seq. ID No. 1621 | Seq. ID No. 1622 |
| 779 | Seq. ID No. 1633 | Seq. ID No. 1634 |
| 786b | Seq. ID No. 1643 | Seq. ID No. 1644 |
| 788a | Seq. ID No. 1645 | Seq. ID No. 1646 |
| 788b | Seq. ID No. 1647 | Seq. ID No. 1648 |
| 789a | Seq. ID No. 1649 | Seq. ID No. 1650 |
| 789c | Seq. ID No. 1653 | Seq. ID No. 1654 |
| 799a | Seq. ID No. 1671 | Seq. ID No. 1672 |
| 799b | Seq. ID No. 1673 | Seq. ID No. 1674 |
| 799d | Seq. ID No. 1677 | Seq. ID No. 1678 |
| 799f | Seq. ID No. 1681 | Seq. ID No. 1682 |
| 826 | Seq. ID No. 1707 | Seq. ID No. 1708 |
| 837a | Seq. ID No. 1731 | Seq. ID No. 1732 |
| 837b | Seq. ID No. 1733 | Seq. ID No. 1734 |
| 843 | Seq. ID No. 1737 | Seq. ID No. 1738 |
| 853a | Seq. ID No. 1753 | Seq. ID No. 1754 |
| 853c | Seq. ID No. 1757 | Seq. ID No. 1758 |
| 859a | Seq. ID No. 1765 | Seq. ID No. 1766 |
| 859b | Seq. ID No. 1767 | Seq. ID No. 1768 |
| 868a | Seq. ID No. 1787 | Seq. ID No. 1788 |
| 870 | Seq. ID No. 1791 | Seq. ID No. 1792 |
| 873c | Seq. ID No. 1797 | Seq. ID No. 1798 |
| 876 | Seq. ID No. 1805 | Seq. ID No. 1806 |
| 885 | Seq. ID No. 1819 | Seq. ID No. 1820 |
| 886 | Seq. ID No. 1821 | Seq. ID No. 1822 |
| 887 | Seq. ID No. 1823 | Seq. ID No. 1824 |
| 908a | Seq. ID No. 1839 | Seq. ID No. 1840 |
| 929a | Seq. ID No. 1869 | Seq. ID No. 1870 |
| 931 | Seq. ID No. 1875 | Seq. ID No. 1876 |
| 939a | Seq. ID No. 1891 | Seq. ID No. 1892 |
| 939b | Seq. ID No. 1893 | Seq. ID No. 1894 |
| 939c | Seq. ID No. 1895 | Seq. ID No. 1896 |
| 952b | Seq. ID No. 1923 | Seq. ID No. 1924 |
| 955a | Seq. ID No. 1927 | Seq. ID No. 1928 |
| 977a | Seq. ID No. 1969 | Seq. ID No. 1970 |
| 981a | Seq. ID No. 1981 | Seq. ID No. 1982 |
| 987 | Seq. ID No. 1991 | Seq. ID No. 1992 |
| 988a | Seq. ID No. 1993 | Seq. ID No. 1994 |
| 998a | Seq. ID No. 2013 | Seq. ID No. 2014 |
| 999a | Seq. ID No. 2017 | Seq. ID No. 2018 |
| 1003b | Seq. ID No. 2027 | Seq. ID No. 2028 |
| 1006a | Seq. ID No. 2033 | Seq. ID No. 2034 |
| 1013a | Seq. ID No. 2041 | Seq. ID No. 2042 |
| 1017a | Seq. ID No. 2049 | Seq. ID No. 2050 |
| 1017c | Seq. ID No. 2053 | Seq. ID No. 2054 |
| 1036a | Seq. ID No. 2087 | Seq. ID No. 2088 |
| 1036b | Seq. ID No. 2089 | Seq. ID No. 2090 |
| 1044a | Seq. ID No. 2105 | Seq. ID No. 2106 |
| 1047b | Seq. ID No. 2115 | Seq. ID No. 2116 |
| 1050a | Seq. ID No. 2121 | Seq. ID No. 2122 |
| 1050d | Seq. ID No. 2127 | Seq. ID No. 2128 |
| 1065b | Seq. ID No. 2151 | Seq. ID No. 2152 |
| 1067 | Seq. ID No. 2159 | Seq. ID No. 2160 |
| 1077a | Seq. ID No. 2171 | Seq. ID No. 2172 |
| 1077b | Seq. ID No. 2173 | Seq. ID No. 2174 |
| 1077d | Seq. ID No. 2177 | Seq. ID No. 2178 |
| 1081a | Seq. ID No. 2187 | Seq. ID No. 2188 |
| 1111a | Seq. ID No. 2237 | Seq. ID No. 2238 |
| 1111b | Seq. ID No. 2239 | Seq. ID No. 2240 |
| 1125 | Seq. ID No. 2245 | Seq. ID No. 2246 |
| 1126b | Seq. ID No. 2249 | Seq. ID No. 2250 |
| 1140a | Seq. ID No. 2271 | Seq. ID No. 2272 |
| 1144a | Seq. ID No. 2283 | Seq. ID No. 2284 |
| 1146a | Seq. ID No. 2289 | Seq. ID No. 2290 |
| 1146b | Seq. ID No. 2291 | Seq. ID No. 2292 |
| 1146c | Seq. ID No. 2293 | Seq. ID No. 2294 |
| 1146d | Seq. ID No. 2295 | Seq. ID No. 2296 |
| 1152a | Seq. ID No. 2303 | Seq. ID No. 2304 |
| 1152b | Seq. ID No. 2305 | Seq. ID No. 2306 |
| 1152c | Seq. ID No. 2307 | Seq. ID No. 2308 |
| 1155a | Seq. ID No. 2317 | Seq. ID No. 2318 |
| 1155b | Seq. ID No. 2319 | Seq. ID No. 2320 |
| 1155c | Seq. ID No. 2321 | Seq. ID No. 2322 |
| 1155d | Seq. ID No. 2323 | Seq. ID No. 2324 |
| 1155e | Seq. ID No. 2325 | Seq. ID No. 2326 |
| 1155f | Seq. ID No. 2327 | Seq. ID No. 2328 |
| 1158b | Seq. ID No. 2333 | Seq. ID No. 2334 |
| 1165 | Seq. ID No. 2343 | Seq. ID No. 2344 |
| 1167a | Seq. ID No. 2347 | Seq. ID No. 2348 |
| 1167b | Seq. ID No. 2349 | Seq. ID No. 2350 |
| 1168 | Seq. ID No. 2351 | Seq. ID No. 2352 |
| 1169a | Seq. ID No. 2353 | Seq. ID No. 2354 |
| 1169b | Seq. ID No. 2355 | Seq. ID No. 2356 |
| 1169c | Seq. ID No. 2357 | Seq. ID No. 2358 |
| 1172 | Seq. ID No. 2365 | Seq. ID No. 2366 |
| 1180d | Seq. ID No. 2389 | Seq. ID No. 2390 |
| 1180e | Seq. ID No. 2391 | Seq. ID No. 2392 |
| 1186a | Seq. ID No. 2407 | Seq. ID No. 2408 |
| 1192a | Seq. ID No. 2419 | Seq. ID No. 2420 |
| 1192b | Seq. ID No. 2421 | Seq. ID No. 2422 |
| 1196b | Seq. ID No. 2427 | Seq. ID No. 2428 |
| 1197 | Seq. ID No. 2429 | Seq. ID No. 2430 |
| 1199a | Seq. ID No. 2433 | Seq. ID No. 2434 |
| 1207 | Seq. ID No. 2449 | Seq. ID No. 2450 |
| 1212 | Seq. ID No. 2459 | Seq. ID No. 2460 |
| 1224b | Seq. ID No. 2489 | Seq. ID No. 2490 |
| 1228a | Seq. ID No. 2497 | Seq. ID No. 2498 |
| 1229c | Seq. ID No. 2503 | Seq. ID No. 2504 |
| 1231 | Seq. ID No. 2507 | Seq. ID No. 2508 |
| 1234a | Seq. ID No. 2515 | Seq. ID No. 2516 |
| 1236a | Seq. ID No. 2523 | Seq. ID No. 2524 |
| 1237 | Seq. ID No. 2525 | Seq. ID No. 2526 |
| 1241a | Seq. ID No. 2531 | Seq. ID No. 2532 |
| 1241c | Seq. ID No. 2535 | Seq. ID No. 2536 |
| 1243 | Seq. ID No. 2537 | Seq. ID No. 2538 |
| 1245a | Seq. ID No. 2541 | Seq. ID No. 2542 |
| 1245b | Seq. ID No. 2543 | Seq. ID No. 2544 |
| 1245c | Seq. ID No. 2545 | Seq. ID No. 2546 |
| 1250a | Seq. ID No. 2555 | Seq. ID No. 2556 |
| 1250b | Seq. ID No. 2557 | Seq. ID No. 2558 |
| 1253a | Seq. ID No. 2569 | Seq. ID No. 2570 |
| 1253b | Seq. ID No. 2571 | Seq. ID No. 2572 |
| 1258a | Seq. ID No. 2583 | Seq. ID No. 2584 |
| 1260 | Seq. ID No. 2591 | Seq. ID No. 2592 |
| 1266a | Seq. ID No. 2597 | Seq. ID No. 2598 |
| 1266b | Seq. ID No. 2599 | Seq. ID No. 2600 |
| 1267a | Seq. ID No. 2601 | Seq. ID No. 2602 |
| 1268b | Seq. ID No. 2607 | Seq. ID No. 2608 |
| 1270 | Seq. ID No. 2611 | Seq. ID No. 2612 |
| 1275a | Seq. ID No. 2621 | Seq. ID No. 2622 |
| 1278b | Seq. ID No. 2629 | Seq. ID No. 2630 |
| 1288 | Seq. ID No. 2651 | Seq. ID No. 2652 |
| 1308a | Seq. ID No. 2679 | Seq. ID No. 2680 |
| 1315 | Seq. ID No. 2693 | Seq. ID No. 2694 |
| 1319a | Seq. ID No. 2701 | Seq. ID No. 2702 |
| 1319b | Seq. ID No. 2703 | Seq. ID No. 2704 |
| 1320a | Seq. ID No. 2705 | Seq. ID No. 2706 |
| 1320b | Seq. ID No. 2707 | Seq. ID No. 2708 |
| 1334b | Seq. ID No. 2731 | Seq. ID No. 2732 |
| 1335a | Seq. ID No. 2733 | Seq. ID No. 2734 |
| 1365b | Seq. ID No. 2783 | Seq. ID No. 2784 |
| 1366a | Seq. ID No. 2785 | Seq. ID No. 2786 |
| 1381b | Seq. ID No. 2813 | Seq. ID No. 2814 |
| 1382 | Seq. ID No. 2815 | Seq. ID No. 2816 |
| 1384c | Seq. ID No. 2823 | Seq. ID No. 2824 |
| 1393a | Seq. ID No. 2835 | Seq. ID No. 2836 |
| 1393b | Seq. ID No. 2837 | Seq. ID No. 2838 |
| 1393d | Seq. ID No. 2841 | Seq. ID No. 2842 |
| 1399a | Seq. ID No. 2853 | Seq. ID No. 2854 |
| 1411a | Seq. ID No. 2873 | Seq. ID No. 2874 |
| 1411b | Seq. ID No. 2875 | Seq. ID No. 2876 |
| 1412b | Seq. ID No. 2881 | Seq. ID No. 2882 |
| 1426a | Seq. ID No. 2905 | Seq. ID No. 2906 |
| 1438 | Seq. ID No. 2935 | Seq. ID No. 2936 |
| 1439a | Seq. ID No. 2937 | Seq. ID No. 2938 |
| 1439b | Seq. ID No. 2939 | Seq. ID No. 2940 |
| 1439c | Seq. ID No. 2941 | Seq. ID No. 2942 |
| 1440a | Seq. ID No. 2947 | Seq. ID No. 2948 |
| 1440b | Seq. ID No. 2949 | Seq. ID No. 2950 |
| 1447a | Seq. ID No. 2969 | Seq. ID No. 2970 |
| 1448 | Seq. ID No. 2973 | Seq. ID No. 2974 |
| 1449b | Seq. ID No. 2977 | Seq. ID No. 2978 |
| 1452a | Seq. ID No. 2981 | Seq. ID No. 2982 |
| 1454 | Seq. ID No. 2987 | Seq. ID No. 2988 |
| 1455b | Seq. ID No. 2991 | Seq. ID No. 2992 |
| 1457a | Seq. ID No. 2995 | Seq. ID No. 2996 |
| 1457b | Seq. ID No. 2997 | Seq. ID No. 2998 |
| 1462b | Seq. ID No. 3005 | Seq. ID No. 3006 |
| 1463a | Seq. ID No. 3007 | Seq. ID No. 3008 |
| 1466a | Seq. ID No. 3013 | Seq. ID No. 3014 |
| 1469a | Seq. ID No. 3023 | Seq. ID No. 3024 |
| 1469b | Seq. ID No. 3025 | Seq. ID No. 3026 |
| 1469c | Seq. ID No. 3027 | Seq. ID No. 3028 |
| 1470a | Seq. ID No. 3029 | Seq. ID No. 3030 |
| 1470b | Seq. ID No. 3031 | Seq. ID No. 3032 |
| 1475 | Seq. ID No. 3043 | Seq. ID No. 3044 |
| 1479c | Seq. ID No. 3055 | Seq. ID No. 3056 |
| 1484a | Seq. ID No. 3063 | Seq. ID No. 3064 |
| 1487 | Seq. ID No. 3071 | Seq. ID No. 3072 |
| 1488a | Seq. ID No. 3073 | Seq. ID No. 3074 |
| 1488b | Seq. ID No. 3075 | Seq. ID No. 3076 |
| 1488c | Seq. ID No. 3077 | Seq. ID No. 3078 |
| 1507c | Seq. ID No. 3123 | Seq. ID No. 3124 |
| 1517a | Seq. ID No. 3145 | Seq. ID No. 3146 |
| 1520 | Seq. ID No. 3155 | Seq. ID No. 3156 |
| 1522 | Seq. ID No. 3157 | Seq. ID No. 3158 |
| 1530a | Seq. ID No. 3163 | Seq. ID No. 3164 |
| 1543 | Seq. ID No. 3183 | Seq. ID No. 3184 |
| 1552 | Seq. ID No. 3193 | Seq. ID No. 3194 |
| 1554b | Seq. ID No. 3201 | Seq. ID No. 3202 |
| 1557a | Seq. ID No. 3213 | Seq. ID No. 3214 |
| 1557b | Seq. ID No. 3215 | Seq. ID No. 3216 |
| 1557c | Seq. ID No. 3217 | Seq. ID No. 3218 |
| 1559a | Seq. ID No. 3219 | Seq. ID No. 3220 |
| 1572a | Seq. ID No. 3241 | Seq. ID No. 3242 |
| 1572b | Seq. ID No. 3243 | Seq. ID No. 3244 |
| 1572c | Seq. ID No. 3245 | Seq. ID No. 3246 |
| 1572e | Seq. ID No. 3249 | Seq. ID No. 3250 |
| 1577b | Seq. ID No. 3257 | Seq. ID No. 3258 |
| 1580a | Seq. ID No. 3261 | Seq. ID No. 3262 |
| 1582b | Seq. ID No. 3269 | Seq. ID No. 3270 |
| 1585a | Seq. ID No. 3275 | Seq. ID No. 3276 |
| 1594a | Seq. ID No. 3297 | Seq. ID No. 3298 |
| 1606a | Seq. ID No. 3317 | Seq. ID No. 3318 |
| 1607b | Seq. ID No. 3323 | Seq. ID No. 3324 |
| 1619 | Seq. ID No. 3337 | Seq. ID No. 3338 |
| 1621 | Seq. ID No. 3339 | Seq. ID No. 3340 |
| 1624 | Seq. ID No. 3349 | Seq. ID No. 3350 |
| 1625a | Seq. ID No. 3351 | Seq. ID No. 3352 |
| 1625b | Seq. ID No. 3353 | Seq. ID No. 3354 |
| 1626a | Seq. ID No. 3357 | Seq. ID No. 3358 |
| 1626b | Seq. ID No. 3359 | Seq. ID No. 3360 |
| 1634c | Seq. ID No. 3377 | Seq. ID No. 3378 |
| 1646a | Seq. ID No. 3389 | Seq. ID No. 3390 |
| 1646b | Seq. ID No. 3391 | Seq. ID No. 3392 |
| 1646d | Seq. ID No. 3395 | Seq. ID No. 3396 |
| 1649a | Seq. ID No. 3405 | Seq. ID No. 3406 |
| 1659a | Seq. ID No. 3421 | Seq. ID No. 3422 |
| 1659c | Seq. ID No. 3425 | Seq. ID No. 3426 |
| 1673a | Seq. ID No. 3449 | Seq. ID No. 3450 |
| 1673b | Seq. ID No. 3451 | Seq. ID No. 3452 |
| 1673c | Seq. ID No. 3453 | Seq. ID No. 3454 |
| 1673d | Seq. ID No. 3455 | Seq. ID No. 3456 |
| 1673e | Seq. ID No. 3457 | Seq. ID No. 3458 |
| 1675a | Seq. ID No. 3463 | Seq. ID No. 3464 |
| 1683a | Seq. ID No. 3481 | Seq. ID No. 3482 |
| 1688a | Seq. ID No. 3491 | Seq. ID No. 3492 |
| 1688b | Seq. ID No. 3493 | Seq. ID No. 3494 |
| 1690a | Seq. ID No. 3503 | Seq. ID No. 3504 |
| 1690b | Seq. ID No. 3505 | Seq. ID No. 3506 |
| 1699a | Seq. ID No. 3527 | Seq. ID No. 3528 |
| 1735a | Seq. ID No. 3593 | Seq. ID No. 3594 |
| 1735b | Seq. ID No. 3595 | Seq. ID No. 3596 |
| 1749a | Seq. ID No. 3621 | Seq. ID No. 3622 |
| 1750a | Seq. ID No. 3625 | Seq. ID No. 3626 |
| 1756b | Seq. ID No. 3635 | Seq. ID No. 3636 |
| 1778b | Seq. ID No. 3679 | Seq. ID No. 3680 |
| 1798a | Seq. ID No. 3723 | Seq. ID No. 3724 |
| 1798c | Seq. ID No. 3727 | Seq. ID No. 3728 |
| 1806b | Seq. ID No. 3745 | Seq. ID No. 3746 |
| 1808a | Seq. ID No. 3753 | Seq. ID No. 3754 |
| 1808b | Seq. ID No. 3755 | Seq. ID No. 3756 |
| 1808c | Seq. ID No. 3757 | Seq. ID No. 3758 |
| 1809a | Seq. ID No. 3761 | Seq. ID No. 3762 |
| 1809b | Seq. ID No. 3763 | Seq. ID No. 3764 |
| 1809c | Seq. ID No. 3765 | Seq. ID No. 3766 |
| 1812b | Seq. ID No. 3783 | Seq. ID No. 3784 |
| 1816a | Seq. ID No. 3793 | Seq. ID No. 3794 |
| 1817a | Seq. ID No. 3799 | Seq. ID No. 3800 |
| 1825 | Seq. ID No. 3813 | Seq. ID No. 3814 |
| 1838a | Seq. ID No. 3837 | Seq. ID No. 3838 |
| 1838b | Seq. ID No. 3839 | Seq. ID No. 3840 |
| 1842a | Seq. ID No. 3849 | Seq. ID No. 3850 |
| 1842b | Seq. ID No. 3851 | Seq. ID No. 3852 |
| 1842c | Seq. ID No. 3853 | Seq. ID No. 3854 |
| 1845a | Seq. ID No. 3859 | Seq. ID No. 3860 |
| 1845b | Seq. ID No. 3861 | Seq. ID No. 3862 |
| 1845c | Seq. ID No. 3863 | Seq. ID No. 3864 |
| 1848a | Seq. ID No. 3865 | Seq. ID No. 3866 |
| 1854a | Seq. ID No. 3879 | Seq. ID No. 3880 |
| 1857b | Seq. ID No. 3889 | Seq. ID No. 3890 |
| 1857c | Seq. ID No. 3891 | Seq. ID No. 3892 |
| 1862a | Seq. ID No. 3903 | Seq. ID No. 3904 |
| 1873c | Seq. ID No. 3925 | Seq. ID No. 3926 |
| 1896a | Seq. ID No. 3963 | Seq. ID No. 3964 |
| 1898a | Seq. ID No. 3973 | Seq. ID No. 3974 |
| 1898b | Seq. ID No. 3975 | Seq. ID No. 3976 |
| 1919a | Seq. ID No. 4005 | Seq. ID No. 4006 |
| 1931a | Seq. ID No. 4025 | Seq. ID No. 4026 |
| 1931b | Seq. ID No. 4027 | Seq. ID No. 4028 |
| 1936 | Seq. ID No. 4039 | Seq. ID No. 4040 |
| 1937a | Seq. ID No. 4041 | Seq. ID No. 4042 |
| 1937b | Seq. ID No. 4043 | Seq. ID No. 4044 |
| 1937c | Seq. ID No. 4045 | Seq. ID No. 4046 |
| 1937d | Seq. ID No. 4047 | Seq. ID No. 4048 |
| 1941b | Seq. ID No. 4061 | Seq. ID No. 4062 |
| 1946a | Seq. ID No. 4071 | Seq. ID No. 4072 |
| 1946b | Seq. ID No. 4073 | Seq. ID No. 4074 |
| 1950 | Seq. ID No. 4079 | Seq. ID No. 4080 |
| 1951a | Seq. ID No. 4081 | Seq. ID No. 4082 |
| 1951b | Seq. ID No. 4083 | Seq. ID No. 4084 |
| 1958a | Seq. ID No. 4101 | Seq. ID No. 4102 |
| 1958b | Seq. ID No. 4103 | Seq. ID No. 4104 |
| 1960c | Seq. ID No. 4111 | Seq. ID No. 4112 |
| 1970b | Seq. ID No. 4133 | Seq. ID No. 4134 |
| 1973 | Seq. ID No. 4137 | Seq. ID No. 4138 |
| 1976 | Seq. ID No. 4139 | Seq. ID No. 4140 |
| 1977c | Seq. ID No. 4145 | Seq. ID No. 4146 |
| 1980a | Seq. ID No. 4151 | Seq. ID No. 4152 |
| 2016a | Seq. ID No. 4221 | Seq. ID No. 4222 |
| 2016b | Seq. ID No. 4223 | Seq. ID No. 4224 |
| 2016c | Seq. ID No. 4225 | Seq. ID No. 4226 |
| 2016d | Seq. ID No. 4227 | Seq. ID No. 4228 |
| 2016f | Seq. ID No. 4231 | Seq. ID No. 4232 |
| 2023a | Seq. ID No. 4241 | Seq. ID No. 4242 |
| 2024b | Seq. ID No. 4247 | Seq. ID No. 4248 |
| 2025 | Seq. ID No. 4253 | Seq. ID No. 4254 |
| 2030a | Seq. ID No. 4257 | Seq. ID No. 4258 |
| 2030c | Seq. ID No. 4261 | Seq. ID No. 4262 |
| 2056 | Seq. ID No. 4307 | Seq. ID No. 4308 |
| 2059a | Seq. ID No. 4311 | Seq. ID No. 4312 |
| 2059b | Seq. ID No. 4313 | Seq. ID No. 4314 |
| 2074b | Seq. ID No. 4343 | Seq. ID No. 4344 |
| 2092 | Seq. ID No. 4379 | Seq. ID No. 4380 |
| 2096a | Seq. ID No. 4385 | Seq. ID No. 4386 |
| 2096c | Seq. ID No. 4389 | Seq. ID No. 4390 |
| 2120b | Seq. ID No. 4439 | Seq. ID No. 4440 |
| 2140a | Seq. ID No. 4469 | Seq. ID No. 4470 |
| 2142a | Seq. ID No. 4473 | Seq. ID No. 4474 |
| 2146 | Seq. ID No. 4481 | Seq. ID No. 4482 |
| 2147a | Seq. ID No. 4483 | Seq. ID No. 4484 |
| 2147b | Seq. ID No. 4485 | Seq. ID No. 4486 |
| 2150c | Seq. ID No. 4497 | Seq. ID No. 4498 |
| 2159b | Seq. ID No. 4515 | Seq. ID No. 4516 |
| 2165 | Seq. ID No. 4533 | Seq. ID No. 4534 |
| 2190a | Seq. ID No. 4587 | Seq. ID No. 4588 |
| 2219a | Seq. ID No. 4639 | Seq. ID No. 4640 |
| 2219c | Seq. ID No. 4643 | Seq. ID No. 4644 |
| 2236a | Seq. ID No. 4681 | Seq. ID No. 4682 |
| 2249 | Seq. ID No. 4705 | Seq. ID No. 4706 |
| 2253a | Seq. ID No. 4707 | Seq. ID No. 4708 |
| 2253b | Seq. ID No. 4709 | Seq. ID No. 4710 |
| 2253c | Seq. ID No. 4711 | Seq. ID No. 4712 |
| 2255a | Seq. ID No. 4715 | Seq. ID No. 4716 |
| 2255b | Seq. ID No. 4717 | Seq. ID No. 4718 |
| 2264b | Seq. ID No. 4737 | Seq. ID No. 4738 |
| 2276a | Seq. ID No. 4759 | Seq. ID No. 4760 |
| 2300 | Seq. ID No. 4813 | Seq. ID No. 4814 |
| 2308 | Seq. ID No. 4827 | Seq. ID No. 4828 |
| 2329a | Seq. ID No. 4869 | Seq. ID No. 4870 |
| 2329b | Seq. ID No. 4871 | Seq. ID No. 4872 |
| 2329c | Seq. ID No. 4873 | Seq. ID No. 4874 |
| 2329d | Seq. ID No. 4875 | Seq. ID No. 4876 |
| 2357 | Seq. ID No. 4919 | Seq. ID No. 4920 |
| 2363b | Seq. ID No. 4931 | Seq. ID No. 4932 |
| 2371a | Seq. ID No. 4949 | Seq. ID No. 4950 |
| 2372 | Seq. ID No. 4951 | Seq. ID No. 4952 |
| 2390 | Seq. ID No. 4987 | Seq. ID No. 4988 |
| 2412 | Seq. ID No. 5031 | Seq. ID No. 5032 |
| 2423a | Seq. ID No. 5061 | Seq. ID No. 5062 |
| 2434 | Seq. ID No. 5085 | Seq. ID No. 5086 |
| 2445 | Seq. ID No. 5103 | Seq. ID No. 5104 |
| 2456c | Seq. ID No. 5123 | Seq. ID No. 5124 |
| 2458a | Seq. ID No. 5129 | Seq. ID No. 5130 |
| 2458b | Seq. ID No. 5131 | Seq. ID No. 5132 |
| 2458d | Seq. ID No. 5135 | Seq. ID No. 5136 |
| 2458e | Seq. ID No. 5137 | Seq. ID No. 5138 |
| 2458g | Seq. ID No. 5141 | Seq. ID No. 5142 |
| 2469b | Seq. ID No. 5165 | Seq. ID No. 5166 |
| 2474b | Seq. ID No. 5175 | Seq. ID No. 5176 |
| 2484b | Seq. ID No. 5199 | Seq. ID No. 5200 |
| 2490 | Seq. ID No. 5205 | Seq. ID No. 5206 |
| 2492a | Seq. ID No. 5207 | Seq. ID No. 5208 |
| 2495a | Seq. ID No. 5213 | Seq. ID No. 5214 |
| 2497b | Seq. ID No. 5219 | Seq. ID No. 5220 |
| 2500a | Seq. ID No. 5223 | Seq. ID No. 5224 |
| 2518a | Seq. ID No. 5267 | Seq. ID No. 5268 |
| 2521 | Seq. ID No. 5281 | Seq. ID No. 5282 |
| 2567a | Seq. ID No. 5385 | Seq. ID No. 5386 |
| 2567b | Seq. ID No. 5387 | Seq. ID No. 5388 |
| 2587a | Seq. ID No. 5413 | Seq. ID No. 5414 |
| 2587b | Seq. ID No. 5415 | Seq. ID No. 5416 |
| 2596 | Seq. ID No. 5433 | Seq. ID No. 5434 |
| 2610 | Seq. ID No. 5453 | Seq. ID No. 5454 |
| 2616a | Seq. ID No. 5459 | Seq. ID No. 5460 |
| 2616b | Seq. ID No. 5461 | Seq. ID No. 5462 |
| 2616c | Seq. ID No. 5463 | Seq. ID No. 5464 |
| 2616d | Seq. ID No. 5465 | Seq. ID No. 5466 |
| 2623b | Seq. ID No. 5491 | Seq. ID No. 5492 |
| 2625a | Seq. ID No. 5497 | Seq. ID No. 5498 |
| 2628 | Seq. ID No. 5509 | Seq. ID No. 5510 |
| 2629b | Seq. ID No. 5513 | Seq. ID No. 5514 |
| 2633d | Seq. ID No. 5525 | Seq. ID No. 5526 |
| 2637b | Seq. ID No. 5531 | Seq. ID No. 5532 |
| 2660 | Seq. ID No. 5569 | Seq. ID No. 5570 |
| 2669a | Seq. ID No. 5587 | Seq. ID No. 5588 |
| 2673 | Seq. ID No. 5595 | Seq. ID No. 5596 |
| 2674a | Seq. ID No. 5597 | Seq. ID No. 5598 |
| 2679a | Seq. ID No. 5609 | Seq. ID No. 5610 |
| 2679b | Seq. ID No. 5611 | Seq. ID No. 5612 |
| 2680c | Seq. ID No. 5621 | Seq. ID No. 5622 |
| 2683a | Seq. ID No. 5623 | Seq. ID No. 5624 |
| 2688a | Seq. ID No. 5633 | Seq. ID No. 5634 |
| 2688c | Seq. ID No. 5637 | Seq. ID No. 5638 |
| 2690a | Seq. ID No. 5643 | Seq. ID No. 5644 |
| 2692b | Seq. ID No. 5649 | Seq. ID No. 5650 |
| 2696a | Seq. ID No. 5659 | Seq. ID No. 5660 |
| 2718b | Seq. ID No. 5683 | Seq. ID No. 5684 |
| 2718c | Seq. ID No. 5685 | Seq. ID No. 5686 |
| 2723 | Seq. ID No. 5699 | Seq. ID No. 5700 |
| 2725b | Seq. ID No. 5705 | Seq. ID No. 5706 |
| 2728a | Seq. ID No. 5709 | Seq. ID No. 5710 |
| 2738a | Seq. ID No. 5729 | Seq. ID No. 5730 |
| 2738c | Seq. ID No. 5733 | Seq. ID No. 5734 |
| 2749a | Seq. ID No. 5763 | Seq. ID No. 5764 |
| 2749b | Seq. ID No. 5765 | Seq. ID No. 5766 |
| 2749c | Seq. ID No. 5767 | Seq. ID No. 5768 |
| 2749e | Seq. ID No. 5771 | Seq. ID No. 5772 |
| 2757b | Seq. ID No. 5783 | Seq. ID No. 5784 |
| 2758d | Seq. ID No. 5791 | Seq. ID No. 5792 |
| 2760a | Seq. ID No. 5797 | Seq. ID No. 5798 |
| 2796a | Seq. ID No. 5885 | Seq. ID No. 5886 |
| 2799a | Seq. ID No. 5889 | Seq. ID No. 5890 |
| 2801a | Seq. ID No. 5893 | Seq. ID No. 5894 |
| 2801b | Seq. ID No. 5895 | Seq. ID No. 5896 |
| 2805a | Seq. ID No. 5899 | Seq. ID No. 5900 |
| 2805b | Seq. ID No. 5901 | Seq. ID No. 5902 |
| 2810a | Seq. ID No. 5905 | Seq. ID No. 5906 |
| 2810c | Seq. ID No. 5909 | Seq. ID No. 5910 |
| 2818a | Seq. ID No. 5921 | Seq. ID No. 5922 |
| 2818b | Seq. ID No. 5923 | Seq. ID No. 5924 |
| 2818d | Seq. ID No. 5927 | Seq. ID No. 5928 |
| 2822a | Seq. ID No. 5939 | Seq. ID No. 5940 |
| 2869c | Seq. ID No. 6027 | Seq. ID No. 6028 |
| 2881 | Seq. ID No. 6043 | Seq. ID No. 6044 |
| 2883a | Seq. ID No. 6047 | Seq. ID No. 6048 |
| 2887a | Seq. ID No. 6059 | Seq. ID No. 6060 |
| 2891a | Seq. ID No. 6067 | Seq. ID No. 6068 |
| 2900b | Seq. ID No. 6089 | Seq. ID No. 6090 |
| 2901a | Seq. ID No. 6091 | Seq. ID No. 6092 |
| 2909b | Seq. ID No. 6107 | Seq. ID No. 6108 |
| 2909d | Seq. ID No. 6111 | Seq. ID No. 6112 |
| 2913b | Seq. ID No. 6117 | Seq. ID No. 6118 |
| 2931a | Seq. ID No. 6161 | Seq. ID No. 6162 |
| 2931b | Seq. ID No. 6163 | Seq. ID No. 6164 |
| 2931c | Seq. ID No. 6165 | Seq. ID No. 6166 |
| 2933b | Seq. ID No. 6169 | Seq. ID No. 6170 |
| 2941a | Seq. ID No. 6183 | Seq. ID No. 6184 |
| 2941b | Seq. ID No. 6185 | Seq. ID No. 6186 |
| 2943a | Seq. ID No. 6189 | Seq. ID No. 6190 |
| 2943b | Seq. ID No. 6191 | Seq. ID No. 6192 |
| 2944b | Seq. ID No. 6197 | Seq. ID No. 6198 |
| 2948a | Seq. ID No. 6203 | Seq. ID No. 6204 |
| 2948b | Seq. ID No. 6205 | Seq. ID No. 6206 |
| 2948d | Seq. ID No. 6209 | Seq. ID No. 6210 |
| 2955a | Seq. ID No. 6221 | Seq. ID No. 6222 |
| 2955b | Seq. ID No. 6223 | Seq. ID No. 6224 |
| 2955c | Seq. ID No. 6225 | Seq. ID No. 6226 |
| 2955d | Seq. ID No. 6227 | Seq. ID No. 6228 |
| 2955e | Seq. ID No. 6229 | Seq. ID No. 6230 |
| 2957 | Seq. ID No. 6235 | Seq. ID No. 6236 |
| 2958a | Seq. ID No. 6237 | Seq. ID No. 6238 |
| 2959 | Seq. ID No. 6243 | Seq. ID No. 6244 |
| 2960a | Seq. ID No. 6245 | Seq. ID No. 6246 |
| 2962b | Seq. ID No. 6249 | Seq. ID No. 6250 |
| 2965a | Seq. ID No. 6253 | Seq. ID No. 6254 |
| 2969 | Seq. ID No. 6263 | Seq. ID No. 6264 |
| 2970a | Seq. ID No. 6265 | Seq. ID No. 6266 |
| 2974a | Seq. ID No. 6273 | Seq. ID No. 6274 |
| 2974b | Seq. ID No. 6275 | Seq. ID No. 6276 |
| 2978b | Seq. ID No. 6285 | Seq. ID No. 6286 |
| 2980a | Seq. ID No. 6289 | Seq. ID No. 6290 |
| 2986a | Seq. ID No. 6301 | Seq. ID No. 6302 |
| 2986b | Seq. ID No. 6303 | Seq. ID No. 6304 |
| 3016a | Seq. ID No. 6363 | Seq. ID No. 6364 |
| 3021 | Seq. ID No. 6377 | Seq. ID No. 6378 |
| 3023 | Seq. ID No. 6381 | Seq. ID No. 6382 |
| 3024a | Seq. ID No. 6383 | Seq. ID No. 6384 |
| 3037a | Seq. ID No. 6415 | Seq. ID No. 6416 |
| 3042b | Seq. ID No. 6427 | Seq. ID No. 6428 |
| 3043b | Seq. ID No. 6431 | Seq. ID No. 6432 |
| 3064b | Seq. ID No. 6473 | Seq. ID No. 6474 |
| 3076 | Seq. ID No. 6497 | Seq. ID No. 6498 |
| 3107a | Seq. ID No. 6571 | Seq. ID No. 6572 |
| 3110a | Seq. ID No. 6581 | Seq. ID No. 6582 |
| 3119a | Seq. ID No. 6593 | Seq. ID No. 6594 |
| 3119d | Seq. ID No. 6599 | Seq. ID No. 6600 |
| 3140 | Seq. ID No. 6631 | Seq. ID No. 6632 |
| 3145c | Seq. ID No. 6639 | Seq. ID No. 6640 |

### B. ALLOIOCOCCUS OTITIDIS ORF POLYNUCLEOTIDES ENCODING SURFACE EXPOSED POLYPEPTIDES

Isolated and purified Alloiococcus otitidis ORF polynucleotides of the present invention are contemplated for use in the production of Alloiococcus otitidis polypeptides. More specifically, in certain embodiments, the ORFs encode Alloiococcus otitidis surface exposed polypeptides, particularly antigenic polypeptides. Thus, in one aspect, the present invention provides isolated and purified polynucleotides (ORFS) that encode Alloiococcus otitidis surface exposed polypeptides. In particular embodiments, a polynucleotide of the present invention is a DNA molecule, wherein the DNA may be genomic DNA, plasmid DNA or cDNA. In a preferred embodiment, a polynucleotide of the present invention is a recombinant polynucleotide, which encodes an Alloiococcus otitidis polypeptide comprising an amino acid sequence that has at least 70% identity to an amino acid sequence of one of the even numbered sequences set forth in SEQ ID NO: 2 through SEQ ID NO: 6650 or a fragment thereof. In another embodiment, an isolated and purified ORF polynucleotide comprises a nucleotide sequence that has at least 70% identity to one of the ORF polynucleotide nucleotide sequences of odd numbered sequences set forth in SEQ ID NO: 1 through SEQ ID NO: 6649, a degenerate variant thereof, or a complement thereof. In yet another embodiment, an ORF polynucleotide of one of the odd numbered sequences set forth in SEQ ID NO: 1 through SEQ ID NO: 6649 is comprised in a plasmid vector and expressed in a host cell. In a preferred embodiment, the host cell is a prokaryotic host cell.

As used herein, the term “polynucleotide” means a sequence of nucleotides connected by phosphodiester linkages. It is well understood that in all prokaryotes studied to date, including Alloiococcus otitidis , most or all cellular genetic material is contained within a single circular chromosome. The single circular chromosome is usually comprised of double stranded DNA, wherein the ORFs encoding polypeptides may be represented on either strand of the bacterial chromosome in a head to tail or a head to head orientation. Polynucleotides are presented herein from the 5′ to the 3′ direction. A polynucleotide of the present invention can comprise from about 10 nucleotides to about several hundred thousand nucleotides. Preferably, a polynucleotide comprises from about 10 to about 3,000 nucleotides. Preferred lengths of particular polynucleotide are set forth hereinafter.

A polynucleotide of the present invention can be a deoxyribonucleic acid (DNA) molecule, a ribonucleic acid (RNA) molecule, or analogs of the DNA or RNA generated using nucleotide analogs. The nucleic acid molecule can be single-stranded or double-stranded, but preferably is double-stranded DNA. Where a polynucleotide is a DNA molecule, that molecule can be a gene, a cDNA molecule or a genomic DNA molecule. Nucleotide bases are indicated herein by a single letter code: adenine (A), guanine (G), thymine (T) and cytosine (C).

“Isolated” means altered “by the hand of man” from the natural state. An “isolated” composition or substance is one that has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is employed herein.

Preferably, an “isolated” polynucleotide is free of sequences, which naturally flank the nucleic acid (ie., sequences located at the 5′ and 3′ ends of the nucleic acid) in the genomic DNA of the organism from which the nucleic acid is derived. For example, in various embodiments, the isolated Alloiococcus otitidis nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g., neuronal or placenta). However, the Alloiococcus otitidis nucleic acid molecule can be fused to heterologous protein encoding or regulatory sequences and still be considered isolated.

ORF polynucleotides of the present invention are obtained using standard cloning and screening techniques from a cDNA library derived from mRNA. Polynucleotides of the invention are also obtained from natural sources such as genomic DNA libraries (e.g., an Alloiococcus otitidis library) or are synthesized using well known and commercially available techniques. As contemplated in the present invention, ORF polynucleotides are obtained using Alloiococcus otitidis chromosomal DNA as the template.

The invention further encompasses nucleic acid molecules that differ from the nucleotide sequences shown in one of the odd numbered sequences set forth in SEQ ID NO: 1 through SEQ ID NO: 6649 (and fragments thereof) due to degeneracy of the genetic code, and thus encode the same Alloiococcus otitidis polypeptide as that encoded by the nucleotide sequence shown in one of the odd numbered sequences set forth in SEQ ID NO:1 through SEQ ID NO: 6649.

Orthologs and allelic variants of the Alloiococcus otitidis polynucleotides are readily identified using methods well known in the art. Allelic variants and orthologs of the polynucleotides comprise a nucleotide sequence that is typically at least about 70-75%, more typically at least about 80-85%, and most typically at least about 90-95% or more homologous to a nucleotide sequence shown in one of the odd numbered sequences set forth in SEQ ID NO:1 through SEQ ID NO: 6649, or a fragment of these nucleotide sequences. Such nucleic acid molecules are readily identified as being able to hybridize, preferably under stringent conditions, to a nucleotide sequence shown in one of the odd numbered sequences set forth in SEQ ID NO:1 through SEQ ID NO: 6649, or a fragment of these nucleotide sequences.

Moreover, the polynucleotides of the invention also comprise only a fragment of the coding region of an Alloiococcus otitidis polynucleotide or gene, such as a fragment of and one of the odd numbered sequences set forth in SEQ ID NO:1 through SEQ ID NO: 6649. Preferably, such fragments encode polypeptides, which are immunogenic fragments.

When the ORF polynucleotides of the invention are used for the recombinant production of Alloiococcus otitidis polypeptides of the present invention, the polynucleotide may include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. For example, a marker sequence, which facilitates purification of the fused polypeptide, can be linked to the coding sequence (see Gentz et al., 1989, incorporated herein by reference). Thus, contemplated in the present invention is the preparation of polynucleotides encoding fusion polypeptides permitting His-tag purification of expression products. The polynucleotide may also contain non-coding 5′ and 3′ sequences, such as transcribed, non-translated sequences, splicing and polyadenylation signals.

Thus, a polynucleotide encoding a polypeptide of the present invention, including homologs and orthologs from species other than Alloiococcus otitidis , is obtained by a process which comprises the steps of screening an appropriate library under stringent hybridization conditions (discussed below) with a labeled probe having the sequence of one of the odd numbered sequences set forth in one of SEQ ID NO:1 through SEQ ID NO: 6649 or a fragment thereof; and isolating full-length cDNA and genomic clones containing the polynucleotide sequence. Such hybridization techniques are well known to the skilled artisan. The skilled artisan will appreciate that, in many cases, an isolated cDNA sequence will be incomplete, in that the region coding for the polypeptide is cut short at the 5′ end of the cDNA. This is a consequence of reverse transcriptase, an enzyme with inherently low “processivity” (a measure of the ability of the enzyme to remain attached to the template during the polymerization reaction), failing to complete a DNA copy of the mRNA template during 1st strand cDNA synthesis.

Thus, in certain embodiments, the polynucleotide sequence information provided by the present invention allows for the preparation of relatively short DNA (or RNA) oligonucleotide sequences having the ability to specifically hybridize to gene sequences of the selected polynucleotides disclosed herein. The term “oligonucleotide” as used herein is defined as a molecule comprised of two or more deoxyribonucleotides or ribonucleotides, usually more than three (3), and typically more than ten (10) and up to one hundred (100) or more (although preferably between twenty and thirty). The exact size will depend on many factors, which in turn depends on the ultimate function or use of the oligonucleotide. Thus, in particular embodiments of the invention, nucleic acid probes of an appropriate length are prepared based on a consideration of a selected nucleotide sequence, e.g., a sequence such as that shown in one of odd numbered sequences set forth in SEQ ID NO:1 through SEQ ID NO: 6649. The ability of such nucleic acid probes to specifically hybridize to a polynucleotide encoding an Alloiococcus otitidis polypeptide lends them particular utility in a variety of embodiments. Most importantly, the probes are used in a variety of assays for detecting the presence of complementary sequences in a given sample.

In certain embodiments, it is advantageous to use oligonucleotide primers. These primers may be generated in any manner, including chemical synthesis, DNA replication, reverse transcription, or a combination thereof. The sequence of such primers is designed using a polynucleotide of the present invention for use in detecting, amplifying or mutating a defined segment of an ORF polynucleotide that encodes an Alloiococcus otitidis polypeptide from prokaryotic cells using polymerase chain reaction (PCR) technology.

In certain embodiments, it is advantageous to employ a polynucleotide of the present invention in combination with an appropriate label for detecting hybrid formation. A wide variety of appropriate labels are known in the art, including radioactive, enzymatic or other ligands, such as avidin/biotin, which are capable of giving a detectable signal.

Polynucleotides which are identical or sufficiently identical to a nucleotide sequence contained in one of odd numbered sequences set forth in SEQ ID NO:1 through SEQ ID NO: 6649, or a fragment thereof, may be used as hybridization probes for cDNA and genomic DNA or as primers for a nucleic acid amplification (PCR) reaction, to isolate full-length cDNAs and genomic clones encoding polypeptides of the present invention and to isolate cDNA and genomic clones of other genes (including genes encoding homologs and orthologs from species other than Alloiococcus otitidis ) that have a high sequence similarity to polynucleotide sequences set forth in one of the odd numbered sequences set forth in SEQ ID NO:1 through SEQ ID NO:6649, or a fragment thereof. Typically these nucleotide sequences are from at least 70% identical to at least about 70% identical to that of the reference polynucleotide sequence. The probes or primers will generally comprise at least 15 nucleotides, preferably, at least 30 nucleotides and may have at least 50 nucleotides. Particularly preferred probes will have between 30 and 50 nucleotides.

There are several methods available and well known to those skilled in the art to obtain full-length cDNAs, or extend short cDNAs, for example those based on the method of Rapid Amplification of cDNA ends (RACE) (see, Frohman et al., 1988). Recent modifications of the technique, exemplified by the Marathon™ technology (BD Biosciences Clontech, Palo Alto, Calif.) for example, have significantly simplified the search for longer cDNAs. In the Marathon™ technology, cDNAs have been prepared from mRNA extracted from a chosen tissue and an “adaptor” sequence ligated onto each end. Nucleic acid amplification (PCR) is then carried out to amplify the “rhissing” 5′ end of the cDNA using a combination of gene specific and adaptor specific oligonucleotide primers. The PCR reaction is then repeated using “nested” primers, that is, primers designed to anneal within the amplified product (typically an adaptor specific primer that anneals further 3′ in the adaptor sequence and a gene specific primer that anneals further 5′ in the known gene sequence). The products of this reaction are then analyzed by DNA sequencing and a full-length cDNA constructed either by joining the product directly to the existing cDNA to give a complete sequence, or carrying out a separate full-length PCR using the new sequence information for the design of the 5′ primer.

To provide certain of the advantages in accordance with the present invention, a preferred nucleic acid sequence employed for hybridization studies or assays includes probe molecules that are complementary to at least a 10 to about 70 long nucleotide stretch of a polynucleotide that encodes an Alloiococcus otitidis polypeptide, such as that shown in one of the even numbered sequences set forth in SEQ ID NO: 2 through SEQ ID NO: 6650. A size of at least 10 nucleotides in length helps to ensure that the fragment will be of sufficient length to form a duplex molecule that is both stable and selective. Molecules having complementary sequences over stretches greater than 10 bases in length are generally preferred in order to increase stability and selectivity of the hybrid, and thereby improve the quality and degree of specific hybrid molecules obtained. It is generally preferred to design nucleic acid molecules with gene-complementary stretches of 25 to 40 nucleotides, 55 to 70 nucleotides, or even longer where desired. For example, such fragments are readily prepared by directly synthesizing the fragment by chemical means, by application of nucleic acid reproduction technology, such as the PCR technology (U.S. Pat. No. 4,683,202, incorporated herein by reference), or by excising selected DNA fragments from recombinant plasmids containing appropriate inserts and suitable restriction enzyme sites.

In another aspect, the present invention contemplates an isolated and purified polynucleotide comprising a nucleotide sequence that is identical or complementary to a segment of at least 10 contiguous bases of one of the odd numbered sequences set forth in SEQ ID NO: 1 through SEQ ID NO: 6649, wherein the polynucleotide hybridizes to a polynucleotide that encodes an Alloiococcus otitidis polypeptide. Preferably, the isolated and purified polynucleotide comprises a base sequence that is identical or complementary to a segment of at least 25 to 70 contiguous bases of one of the odd numbered sequences set forth in SEQ ID NO: 1 through SEQ ID NO: 6649. For example, the polynucleotide of the invention can comprise a segment of bases identical or complementary to from 40 to 55 contiguous bases of the disclosed nucleotide sequences.

Accordingly, a polynucleotide probe molecule of the invention is used for its ability to selectively form duplex molecules with complementary stretches of the gene. Depending on the application envisioned, one will desire to employ varying conditions of hybridization stringency to achieve varying degree of selectivity of the probe toward the target sequence (see Table 22 below). For applications requiring a high degree of selectivity, one will typically desire to employ relatively stringent conditions to form the hybrids. For some applications, for example, where one desires to prepare mutants employing a mutant primer strand hybridized to an underlying template or where one seeks to isolate an Alloiococcus otitidis homologous polypeptide coding sequence from other cells, functional equivalents, or the like, less stringent hybridization conditions are typically needed to allow formation of the heteroduplex (see Table 22). Cross-hydridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

The present invention also includes polynucleotides capable of hybridizing under reduced stringency conditions, more preferably stringent conditions, and most preferably highly, stringent conditions, to polynucleotides described herein. Examples of stringency conditions are shown in the Table 22 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 22
HYBRIDIZATION STRINGENCY CONDITIONS

| | | | Hybridization | |
|---|---|---|---|---|
| Stringency | Polynucleotide | Hybrid | Temperature | Wash Temperature |
| Condition | Hybrid | Length (bp)¹ | and Buffer^{H} | and Buffer^{H} |
| A | DNA:DNA | >50 | 65° C.; 1 × SSC -or- | 65° C.; 0.3 × SSC |
| | | | 42° C.; 1 × SSC, | |
| | | | 50% formamide | |
| B | DNA:DNA | <50 | T_{B}; 1 × SSC | T_{B}; 1 × SSC |
| C | DNA:RNA | >50 | 67° C.; 1 × SSC - | 67° C.; 0.3 × SSC |
| | | | or- | |
| | | | 45° C.; 1 × SSC, | |
| | | | 50% formamide | |
| D | DNA:RNA | <50 | T_{D}; 1 × SSC | T_{D}; 1 × SSC |
| E | RNA:RNA | >50 | 70° C.; 1 × SSC - | 70° C.; 0.3 × SSC |
| | | | or- | |
| | | | 50° C.; 1 × SSC, | |
| | | | 50% formamide | |
| F | RNA:RNA | <50 | T_{F}; 1 × SSC | T_{F}; 1 × SSC |
| G | DNA:DNA | >50 | 65° C.; 4 × SSC - | 65 0; 1 × SSC |
| | | | or- | |
| | | | 42° C.; 4 × SSC, | |
| | | | 50% formamide | |
| H | DNA:DNA | <50 | T_{H}; 4 × SSC | T_{H}; 4 × SSC |
| I | DNA:RNA | >50 | 67° C.; 4 × SSC - | 67° C.; 1 × SSC |
| | | | or- | |
| | | | 45° C.; 4 × SSC, | |
| | | | 50% formamide | |
| J | DNA:RNA | <50 | T_{J}; 4 × SSC | T_{J}; 4 × SSC |
| K | RNA:RNA | >50 | 70° C.; 4 × SSC - | 67° C.; 1 × SSC |
| | | | or- | |
| | | | 50° C.; 4 × SSC, | |
| | | | 50% formamide | |
| L | RNA:RNA | <50 | T_{L}; 2 × SSC | T_{L}; 2 × SSC |
| M | DNA:DNA | >50 | 50° C.; 4 × SSC - | 50° C.; 2 × SSC |
| | | | or- | |
| | | | 40° C.; 6 × SSC, | |
| | | | 50% formamide | |
| N | DNA:DNA | <50 | T_{N}; 6 × SSC | T_{N}; 6 × SSC |
| O | DNA:RNA | >50 | 55° C.; 4 × SSC - | 55° C.; 2 × SSC |
| | | | or- | |
| | | | 42° C.; 6 × SSC, | |
| | | | 50% formamide | |
| P | DNA:RNA | <50 | T_{P}; 6 × SSC | T_{P}; 6 × SSC |
| Q | RNA:RNA | >50 | 60° C.; 4 × SSC - | 60° C.; 2 × SSC |
| | | | or- | |
| | | | 45° C.; 6 × SSC, | |
| | | | 50% formamide | |
| R | RNA:RNA | <50 | T_{R}; 4 × SSC | T_{R}; 4 × SSC |
| (bp)¹: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid | | | | |
| # length is determined by aligning the sequences of the polynucleotides and identifying the region or regions of optimal sequence complementarity. | | | | |
| Buffer^{H}: SSPE (1 × SSPE is 0.15 M NaCl, 10 mM NaH₂PO₄, and 1.25 mM EDTA, pH 7.4), can be substituted for SSC (1 × SSC is 0.15 M NaCl and 15 mM sodium citrate) in the hybridization and wash buffers; washes are performed for 15 minutes after hybridization is complete. | | | | |
| T_{B} through T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). | | | | |
| # For hybrids between 18 and 49 base pairs in length, Tₘ(° C.) = 81.5 + 16.6(log₁₀[Na⁺]) + 0.41 (% G + C) − (600/N), where N is the number of bases in the hybrid, and [Na⁺] is the concentration of sodium ions in the hybridization buffer ([Na⁺] for 1 × SSC = 0.165 M). | | | | |

Additional examples of stringency conditions for polynucleotide hybridization are provided in Sambrook et al., 1.989, Molecular Cloning: A Laboratory Manual, Cold Spring Harbor Laboratory Press, Cold Spring Harbor, N.Y., chapters 9 and 11, and Ausubel et al., 1995, Current Protocols in Molecular Biology, Eds., John Wiley & Sons, Inc., sections 2.10 and 6.3-6.4, incorporated herein by reference.

In addition to the nucleic acid molecules encoding Alloiococcus otitidis polypeptides described above, another aspect of the invention pertains to isolated nucleic acid molecules that are antisense thereto. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire Alloiococcus otitidis coding strand, or to only a fragment thereof. In one embodiment, an antisense nucleic acid molecule is antisense to a “coding region” of the coding strand of a nucleotide sequence encoding an Alloiococcus otitidis polypeptide.

The term “coding region” refers to the region of the nucleotide sequence comprising codons which are translated into amino acid residues, e.g., the entire coding region of each of odd numbered sequences set forth in SEQ ID NO: 1 through SEQ ID NO: 6649. In another embodiment, the antsense nucleic acid molecule is antisense to a “noncoding region” of the coding strand of a nucleofide sequence encoding an Alloiococcus otitidis polypeptide. The term “noncoding region” refers to 5′ and 3′ sequences that flank the coding region that are not translated into amino acids (i.e., also referred to as 5′ and 3′ untranslated regions).

Given the coding strand sequence encoding the Alloiococcus otitidis polypeptides disclosed herein antisense nucleic acids of the invention can be designed according to the rules of Watson and Crick base pairing. The antisense nucleic acid molecule can be complementary to the entire coding region of Alloiococcus otitidis mRNA, but more preferably is an oligonucleotide which is antisense to only a fragment of the coding or noncoding region of Alloiococcus otitidis mRNA. For example, the antisense oligonucleotide can be complementary to the region surrounding the translation start site of Alloiococcus otitidis mRNA.

An antisense oligonucleotide can be, for example, about 5, 10, 15,20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an Alloiococcus otitidis polypeptide to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual γ-units, the strands run parallel to each other (Gaultier et al., 1987). The antisense nucleic acid molecule can also comprise a 2′-o-methylribonucleotide (Inoue et al., 1987a) or a chimeric RNA-DNA analogue (Inoue et al., 1987b).

In still another embodiment, an antisense nucleic acid of the invention is a ribozyme. Ribozymes are catalytic RNA molecules with ribonuclease activity that are capable of cleaving a single-stranded nucleic acid, such as a mRNA, to which they have a complementary region. Thus, ribozymes (e.g., hammerhead ribozymes described in Haselhoff and Gerlach, 1988) can be used to catalytically cleave Alloiococcus otitidis mRNA transcripts to thereby inhibit translation of Alloiococcus otitidis mRNA. A ribozyme having specificity for an Alloiococcus otitidis-encoding nucleic acid can be designed based upon the nucleotide sequence of an Alloiococcus otitidis cDNA disclosed herein. For example, a derivative of a Tetrahymena L-19 IVS RNA can be constructed in which the nucleotide sequence of the active site is complementary to the nucleotide sequence to be cleaved in an Alloiococcus otitidis-encoding mRNA. See, e.g., Cech et al. U.S. Pat. No. 4,987,071 and Cech et al. U.S. Pat. No. 5,116,742 both incorporated herein in their entirety by reference. Altematively, Alloiococcus otitidis mRNA can be used to select a catalytic RNA having a specific ribonuclease activity from a pool of RNA molecules. See, e.g., Bartel and Szostak, 1993.

Alternatively Alloiococcus otitidis gene expression is inhibited by targeting nucleotide sequences complementary to the regulatory region of the Alloiococcus otitidis gene (e.g., the Alloiococcus otitidis gene promoter and/or enhancers) to form triple helical structures that prevent transcription of the Alloiococcus otitidis gene in target cells. (See generally, Helene, 1991; Helene et al., 1992; and Maher, 1992).

Alloiococcus otitidis gene expression is also inhibited using RNA interference (RNAi). This is a technique for post-transcriptional gene silencing (PTGS), in which target gene activity is specifically abolished with cognate double-stranded RNA (dsRNA). RNAi resembles in many aspects PTGS in plants and has been detected in many invertebrates including trypanosome, hydra, planaria, nematode and fruit fly (Drosophila melangnoste). It may be involved in the modulation of transposable element mobilization and antiviral state formation. RNAi in mammalian systems is disclosed in WO 00/63364, which is incorporated by reference herein in its entirety. Basically, dsRNA of at least about 600 nucleotides, homologous to the target is introduced into the cell and a sequence specific reduction in gene activity is observed.

### C. ALLOIOCOCCUS OTITIDIS POLYPEPTIDES

In particular embodiments, the present invention provides isolated and purified Alloiococcus otitidis polypeptides. Preferably, an Alloiococcus otitidis polypeptide of the invention is a recombinant polypeptide. In certain embodiments, an Alloiococcus otitidis polypeptide of the present invention comprises the amino acid sequence that has at least 70% identity to the amino acid sequence of one of even numbered sequences set forth in SEQ ID NO: 2 through SEQ ID NO: 6650, a biological equivalent thereof, or a fragment thereof.

An Alloiococcus otitidis polypeptide according to the present invention encompasses a polypeptide that comprises: 1) the amino acid sequence shown in one of the even numbered sequences set forth in SEQ ID NO: 2 through SEQ ID NO: 6650); (2) functional and non-functional naturally occurring variants or biological equivalents of Alloiococcus otitidis polypeptides of in one of the even numbered sequences set forth in SEQ ID NO: 2 through SEQ ID NO: 6650; (3) recombinantly produced variants or biological equivalents of Alloiococcus otitidis polypeptides set out in one of the even numbered sequences set forth in SEQ ID NO: 2 through SEQ ID NO: 6650; (4) polypeptides isolated from organisms other than Alloiococcus otitidis (orthologs of Alloiococcus otitidis polypeptides).

A biological equivalent or variant of an Alloiococcus otitidis polypeptide according to the present invention encompasses 1) a polypeptide isolated from Alloiococcus otitidis, and 2) a polypeptide that contains substantial homology to an Alloiococcus otitidis polypeptide.

Biological equivalents or variants of Alloiococcus otitidis include both functional and non-functional Alloiococcus otitidis polypeptides. Functional biological equivalents or variants are naturally occurring amino acid sequence variants of an Alloiococcus otitidis polypeptide that maintain the ability to elicit an immunological or antigenic response in a subject. Functional variants will typically contain only conservative substitutions of one or more amino acids in one of the even numbered sequences set forth in SEQ ID NO: 2 through SEQ ID NO: 6650 or substitution, deletion or insertion of non-critical residues in non-critical regions of the polypeptide (e.g., not in regions containing antigenic determinants or protective epitopes).

The present invention further provides non-Alloiococcus otitidis orthologs of Alloiococcus otitidis polypeptides. Orthologs of Alloiococcus otitidis polypeptides are polypeptides that are isolated from non-Alloiococcus otitidis organisms and possess antigenic capabilities of the Alloiococcus otitidis polypeptide. Orthologs of an Alloiococcus otitidis polypeptide can readily be identified as comprising an amino acid sequence that is substantially homologous to one of the even numbered sequences set forth in set forth in SEQ ID NO: 2 through SEQ ID NO: 6650.

Modifications and changes can be made in the structure of a polypeptide of the present invention and still obtain a molecule having Alloiococcus otitidis antigenicity. For example, certain amino acids can be substituted for other amino acids in a sequence without appreciable loss of antigenicity. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide with like properties.

In making such changes, the hydropathic index of amino acids can be considered. The importance of the hydropathic amino acid index in conferring interactive biologic function on a polypeptide is generally understood in the art (Kyte & Doolittle, 1982). It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biologically functional equivalent polypeptide or peptide thereby created is intended for use in immunological embodiments. U.S. Pat. No. 4,554,101, incorporated herein by reference, states that the greatest local average hydrophilicity of a polypeptide, as governed by the hydrophilicity of its adjacent amino acids, correlates with its immunogenicity and antigenicity, i.e. with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those that are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (see Table 23, below). The present invention thus contemplates functional or biological equivalents of an Alloiococcus otitidis polypeptide as set forth above.

TABLE 23
AMINO ACID SUBSTITUTIONS

| Original | Exemplary Residue |
|---|---|
| Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Met; Leu; Tyr |
| Ser | Thr |
| Thr | Ser |
| Trp | Tyr |
| Tyr | Trp; Phe |
| Val | Ile; Leu |

Biological or functional equivalents of a polypeptide are also prepared using site-specific mutagenesis. Site-specific mutagenesis is a technique useful in the preparation of second generation polypeptides, or biologically functional equivalent polypeptides or peptides, derived from the sequences thereof, through specific mutagenesis of the underlying DNA. As noted above, such changes can be desirable where amino acid substitutions are desirable. The technique further provides a capacity to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the site of the alteration of the sequence.

In general, the technique of site-specific mutagenesis is well known in the art. As will be appreciated, the technique typically employs a phage vector that can exist in both a single stranded and double stranded form. Typically, site-directed mutagenesis in accordance herewith is performed by first obtaining a single-stranded vector which includes within its sequence a DNA sequence which encodes all or a portion of the Alloiococcus otitidis polypeptide sequence selected. An oligonucleotide primer bearing the desired mutated sequence is prepared (e.g., synthetically). This primer is then annealed to the singled-stranded vector, and extended by the use of enzymes such as Escherichia coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells such as Escherichia coli cells and clones are selected which include recombinant vectors bearing the mutation. Commercially available kits come with all the reagents necessary, except the oligonucleotide primers.

An Alloiococcus otitidis polypeptide or polypeptide antigen of the present invention is understood to mean any Alloiococcus otitidis polypeptide comprising substantial sequence similarity, structural similarity and/or functional similarity to an Alloiococcus otitidis polypeptide comprising the amino acid sequence of one of the even numbered sequences set forth in SEQ ID NO: 2 through SEQ ID NO: 6650. In addition, an Alloiococcus otitidis polypeptide or polypeptide antigen of the invention is not limited to a particular source. Thus, the invention provides for the general detection and isolation of the polypeptides from a variety of sources.

It is contemplated in the present invention, that an Alloiococcus otitidis polypeptide may advantageously be cleaved into fragments for use in further structural or functional analysis, or in the generation of reagents such as Alloiococcus otitidis-related polypeptides and Alloiococcus otitidis-specific antibodies. This can be accomplished by treating purified or unpurified Alloiococcus otitidis polypeptides with a peptidase such as endoproteinase glu-C (Roche Diagnostics Corporation, Indianapolis, Ind.). Treatment with CNBr is another method by which peptide fragments may be produced from natural Alloiococcus otitidis polypeptides. Recombinant techniques also can be used to produce specific fragments of an Alloiococcus otitidis polypeptide.

In addition, the Applicants also contemplate that compounds sterically similar to a particular Alloiococcus otitidis polypeptide antigen, called peptidomimetics, may be formulated to mimic the key portions of the peptide structure. Peptidemimetics are peptide-containing molecules that mimic elements of protein secondary structure. (See, for example, Johnson et al., 1993.) The underlying rationale behind the use of peptide mimetics is that the peptide backbone of proteins exists chiefly to orient amino acid side chains in such a way as to facilitate molecular interactions, such as those of receptor and ligand.

Successful applications of the peptide mimetic concept have thus far focused on mimetics of β-turns within proteins. Likely β-turn structures within Alloiococcus otitidis proteins can be predicted by computer-based algorithms as discussed above. Once the component amino acids of the turn are determined, mimetics can be constructed to achieve a similar spatial orientation of the essential elements of the amino acid side chains, as discussed in Johnson et al., 1993.

Fragments of the Alloiococcus otitidis polypeptides are also included in the invention. A fragment is a polypeptide having an amino acid sequence that entirely is the same as a part, but not all, of the amino acid sequence. The fragment can comprise, for example, at least 7 or more (e.g., 8, 10 12, 14,16, 18, 20 or more) contiguous amino acids of an one of amino acid sequence selected from one of the even numbered sequences set forth in SEQ ID NO.: 2 through SEQ ID NO.: 6650. Fragments may be “freestanding” or comprised within a larger polypeptide of which they form a part or region, most preferably as a single, continuous region. In one embodiment, the fragments include at least one epitope of the mature polypeptide sequence. “Fusion protein” refers to a protein encoded by two, often unrelated, fused genes or fragments thereof. For example, fusion proteins comprising various portions of constant region of immunoglobulin molecules together with another human protein or part thereof have been described. In many cases, employing an immunoglobulin Fc region as a part of a fusion protein is advantageous for use in therapy and diagnosis resulting in, for example, improved pharmacokinetic properties (see, e.g., EP-A 0232 2621). On the other hand, for some uses it would be desirable to be able to delete the Fc part after the fusion protein has been expressed, detected and purified.

### D. ALLOIOCOCCUS OTITIDIS POLYNUCLEOTIDE AND POLYPEPTIDE VARIANTS

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions and deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Preferred methods to determine identity are designed to give the largest match between the sequences tested. Methods to determine identity are codified in publicly available computer programs. Preferred computer program methods to determine identity between two sequences include, but are not limited to, the GCG program package (Devereux, J., et al 1984), BLASTP, BLASTN, and FASTA (Altschul, S. F., et al., 1990. The BLASTX program is publicly available from NCBI and other sources (BLAST Manual, Altschul, S., et al., NCBI NLM NIH Bethesda, Md. 20894; Altschul, S., et al., 1990). The well-known Smith-Waterman algorithm may also be used to determine identity. (Smith, T. L. and Waterman, M. S., 1981).

By way of example, a polynucleotide sequence of the present invention may be identical to the reference sequence of one of the odd numbered sequences set forth in SEQ ID NO:1 through SEQ ID NO: 6649, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in one of the odd numbered sequences set forth in SEQ ID NO: 1 through SEQ ID NO: 6649 by the numerical percent of the respective percent identity (divided by 100) and subtracting that product from said total number of nucleotides in one of the odd numbered sequences set forth in SEQ ID NO: 1 through SEQ ID NO: 6649.

For example, the alterations in an isolated Alloiococcus otitidis polynucleotide comprising a polynucleotide sequence that has at least 70% identity to the nucleic acid sequence of one of the odd numbered sequences set forth in SEQ ID NO: 1 through SEQ ID NO: 6649; a degenerate variant thereof or a fragment thereof, wherein the polynucleotide sequence may include up to nₙ nucleic acid alterations over the entire polynucleotide region of the nucleic acid sequence of one of the odd numbered sequences set forth in SEQ ID NO: 1 through SEQ ID NO: 6649, wherein nₙ is the maximum number of alterations and is calculated by the formula:
nₙ≦xₙ−(xₙ·y),
in which xₙ is the total number of nucleic acids of one of the odd numbered sequences set forth in SEQ ID NO:1 through SEQ ID NO:6649 and y has a value of 0.70, wherein any non-integer product of xₙ and y is rounded down to the nearest integer prior to subtracting such product from xₙ. Furthermore, y may also have a value of 0.80 for 80%, 0.85 for 85%, 0.90 for 90% 0.95 for 95%, etc.

Similarly, a polypeptide sequence of the present invention may be identical to the reference sequence of one of the even numbered sequences set forth in SEQ ID NO: 2 through SEQ ID NO: 6650, that is 100% identical, or it may include up to a certain integer number of amino acid alterations as compared to the reference sequence such that the percentage identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in one of the even numbered sequences set forth in SEQ ID NO: 2 through SEQ ID NO: 6650 by the numerical percent of the respective percent identity (divided by 100) and then subtracting that product from said total number of amino acids in one of the even numbered sequences set forth in SEQ ID NO: 2 through SEQ ID NO: 6650, or:
nₐ≦xₐ−(xₐ·y),
wherein nₐ is the number of amino acid alterations, xₐ is the total number of amino acids in one of the even numbered sequences set forth in SEQ ID NO: 2 through SEQ ID NO: 6650, and y is, for instance 0.70 for 70%, 0.80 for 80%, 0.85 for 85% etc., and wherein any non-integer product of xₐ and y is rounded down to the nearest integer prior to subtracting it from xₐ.
E. VECTORS, HOST CELLS AND RECOMBINANT ALLOIOCOCCUS OTITIDIS POLYPEPTIDES

In a preferred embodiment, the present invention provides expression vectors comprising ORF polynucleotides that encode Alloiococcus otitidis polypeptides. Preferably, the expression vectors of the present invention comprise ORF polynucleotides that encode Alloiococcus otitidis polypeptides comprising the amino acid residue sequence of one of the even numbered sequences set forth in SEQ ID NO: 2 through SEQ ID NO: 6650. More preferably, the expression vectors of the present invention comprise a polynucleotide comprising the nucleotide base sequence of one of the odd numbered sequences set forth in SEQ ID NO: 1 through SEQ ID NO: 6649. Even more preferably, the expression vectors of the invention comprise a polynucleotide operatively linked to a promoter. Still more preferably, the expression vectors of the invention comprise a polynucleotide operatively linked to a prokaryotic promoter. Alternatively, the expression vectors of the present invention comprise a polynucleotide operatively linked to an enhancer-promoter, that is, an eukaryotic promoter. The expression vectors further comprise a polyadenylation signal that is positioned 3′ of the codon encoding the carboxy-terminal amino acid and within a transcriptional unit of the encoded polypeptide.

Expression of proteins in prokaryotes is most often carried out in Escherichia coil with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc;), pMAL (New England Biolabs, Beverly; Mass.) and pRIT5 (Pharmacia, Piscataway, N.J.) which fuse glutathione S-transferase (GST), maltose E binding protein, or protein A, respectively, to the target recombinant protein.

In one embodiment, the coding sequence of the Alloiococcus otitidis polynucleotide is cloned into a pGEX expression vector to create a vector encoding a fusion protein comprising, from the N-terminus to the C-terminus, GST-thrombin cleavage site-Alloiococcus otitidis polypeptide. The fusion protein is purified by affinity chromatography using glutathione-agarose resin. Recombinant Alloiococcus otitidis polypeptide unfused to GST is recovered by cleavage of the fusion protein with thrombin.

Examples of suitable inducible non-fusion Escherichia coli expression vectors include pTrc (Amann et al., 1988) and pET I I d (Studier et al., 1990). Target gene expression from the pTrc vector relies on host RNA polymerase transcription from a hybrid trp-lac fusion promoter. Target gene expression from the pET II vector relies on transcription from a T7 gene1 lac fusion promoter mediated by a coexpressed viral RNA polymerase T7 genel. This viral polymerase is supplied by host strains BL21 (DE3) or HMS I 74(DE3) from a resident prophage harboring a T7 gene1 gene under the transcriptional control of the lacUV 5 promoter.

One strategy to maximize recombinant protein expression in Escherichia coli is to express the protein in a host bacterium with an impaired capacity to proteolytically cleave the recombinant protein. Another strategy is to alter the nucleic acid sequence of the nucleic acid to be inserted into an expression vector so that the individual codons for each amino acid are those preferentially utilized in Escherichia coli. Such alteration of nucleic acid sequences of the invention can be carried out by standard DNA mutagenesis or synthesis techniques.

In another embodiment, the Alloiococcus otitidis polynucleotide expression vector is a yeast expression vector. Examples of vectors for expression in a yeast such as S. cerevisiae include pYepSec I (Baldari, et al., 1987), pMFa (Kurjan and Herskowitz, 1982), pJRY88 (Schultz et al., 1987), and pYES2 (Invitrogen Corporation, San Diego, Calif.).

Alternatively, an Alloiococcus otitidis polynucleotide is expressed in insect cells using, for example, baculovirus expression vectors. Baculovirus vectors available for expression of proteins in cultured insect cells (e.g., Sf 9 or Sf 21 cells) include the pAc series (Smith et al., 1983) and the pVL series (Lucklow and Summers, 1989).

In yet another embodiment, a nucleic acid of the invention is expressed in mammalian cells using a mammalian expression vector. Examples of mammalian expression vectors include pCDM8 (Seed, 1987) and pMT2PC (Kaufman et al., 1987). When used in mammalian cells, the expression vector's control functions are often provided by viral regulatory elements.

For example, commonly used promoters are derived from polyoma, Adenovirus 2, cytomegalovirus (CMV) and Simian Virus 40 (SV40). For other suitable expression systems for both prokaryotic and eukaryotic cells see chapters 16 and 17 of Sambrook et al., “Molecular Cloning: A Laboratory Manual” 2nd, ed, Cold Spring Harbor Laboratory, Cold Spring Harbor Laboratory Press, Cold Spring Harbor, N.Y., 1989, incorporated herein by reference.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987), lymphoid-specific promoters (Calame and Eaton, 1988), in particular promoters of T cell receptors (Winoto and Baltimore, 1989) and immunoglobulins (Banerli et al., (1983); and Queen and Baltimore (1983)), neuron-specific promoters (e.g., the neurofilament promoter; Byrne and Ruddle, 1989), pancreas-specific promoters (Edlund et al., 1985), and mammary gland-specific promoters (e.g., milk whey promoter; U.S. Pat. No. 4, 873,316 and EP 264,166). Developmentally-regulated promoters are also encompassed, for example the murine hox promoters (Kessel and Gruss, 1990) and the α-fetoprotein promoter (Campes and Tilghman, 1989).

The invention further provides a recombinant expression vector comprising a DNA molecule encoding an Alloiococcus otitidis polypeptide cloned into the expression vector in an antisense orientation. That is, the DNA molecule is operatively linked to a regulatory sequence in a manner which allows for expression (by transcription of the DNA molecule) of an RNA molecule which is antisense to Alloiococcus otitidis mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector cari be in the form of a recombinant plasmid, phagemid or attenuated virus in which antisense nucleic acids are produced under the control of a high efficiency regulatory region, the activity of which can be determined by the cell type into which the vector is introduced.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, an Alloiococcus otitidis polypeptide can be expressed in bacterial cells such as Escherichia coli, insect cells, yeast or mammalian cells (such as Chinese hamster ovary cells (CHO), NIH3T3, PER C6, NSO or COS cells). Other suitable host cells are known to those skilled in the art.

Vector DNA can be introduced into prokaryotc or eukaryotic cells via conventional transformation, infection or transfection techniques. As used herein, the terms “transformation” and “transfection” are intended to refer to a variety of art-recongnized techniques for introducing foreign nucleic acid (e.g., DNA) into a host cell, including calcium phosphate or calcium chloride co-precipitation, DEAE-dextran-mediated transfection, lipofection, protoplast fusion, direct microinfection. Another recognized technique for introducing DNA into a host cell is “infection”, such as by adenovirus infection or electroporation. Suitable methods for transforming, infecting or transfecting host cells can be found in Sambrook, et al. (“Molecular Cloning: A Laboratory Manual” 2nd ed, Cold Spring Harbor Laboratory, Cold Spring Harbor Laboratory Press, Cold Spring Harbor, N.Y., 1989), and other laboratory manuals.

The most widely used method is transfection mediated by either calcium phosphate or DEAE-dextran. Although the mechanism remains unclear, it is believed that the transfected DNA enters the cytoplasm of the cell by endocytosis and is transported to the nucleus. Depending on the cell type, up to 90% of a population of cultured cells can be transfected at any one time. Because of its high efficiency, transfection mediated by calcium phosphate or DEAE-dextran is the method of choice for experiments that require transient expression of the foreign DNA in large numbers of cells. Calcium phosphate-mediated transfection is also used to establish cell lines that integrate copies of the foreign DNA, which are usually arranged in head-to-tail tandem arrays into the host cell genome.

In the protoplast fusion method, protoplasts derived from bacteria carrying high numbers of copies of plasmid of interest are mixed directly with cultured mammalian cells. After fusion of the cell membranes (usually with polyethylene glycol), the contents of the bacteria are delivered into the cytoplasm of the mammalian cells and the plasmid DNA is transported to the nucleus. Protoplast fusion is not as efficient as transfection for many of the cell lines that are commonly used for transient expression assays, but it is useful for cell lines in which endocytosis of DNA occurs inefficiently. Protoplast fusion frequently yields multiple copies of the plasmid DNA tandemly integrated into the host chormosome.

The application of brief, high-voltage electric pulses (electroporation) to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of cloned genes and for establishment of cell lines that carry integrated copies of the gene of interest. Electroporation, in contrast to calcium phosphate-mediated transfection and protoplast fusion, frequently gives rise to cell lines that carry one, or at most a few, integrated copies of the foreign DNA.

Liposome transfection involves encapsulation of DNA and RNA within liposomes, followed by fusion of the liposomes with the cell membrane. The mechanism of how DNA is delivered into the cell is unclear, but transfection efficiencies can be as high as 90%.

Direct microinjection of a DNA molecule into nuclei has the advantage of not exposing DNA to cellular compartments such as low-pH endosomes. Microinjection therefore used primarily as a method to establish lines of cells that carry integrated copies of the DNA of interest.

The use of adenovirus as a vector for cell transfection is well known in the art. Adenovirus vector-mediated cell transfection has been reported for various cells (Stratford-Perricaudet, et al. 1992).

A host cell of the invention, such as a prokaryotic or eukaryotic host cell in culture, is used to produce (ie., express) one or more Alloiococcus otitidis polypeptides. Accordingly, the invention further provides methods for producing an Alloiococcus otitidis polypeptide using the host cells of the invention. In one embodiment, the method comprises culturing the host cell of invention (into which a recombinant expression vector encoding an Alloiococcus otitidis polypeptide has been introduced) in a suitable medium until the Alloiococcus otitidis polypeptide is produced. In another embodiment, the method further comprises isolating the Alloiococcus otitidis polypeptide from the medium or the host cell.

As used herein, a promoter is a region of a DNA molecule typically within about 100 nucleotide pairs in front of (upstream of) the point at which transcription begins (i.e., a transcription start site). That region typically contains several types of DNA sequence elements that are located in similar relative positions in different genes. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

As used herein, the phrase “enhancer-promoter” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Means for operatively linking an enhancer-promoter to a coding sequence are well known in the art. As is also well known in the art, the precise orientation and location relative to a coding sequence whose transcription is controlled, is dependent inter alia upon the specific nature of the enhancer-promoter. Thus, a TATA box minimal promoter is typically located from about 25 to about 30 base pairs upstream of a transcription initiation site and an upstream promoter element is typically located from about 100 to about 200 base pairs upstream of a transcription initiation site. In contrast, an enhancer can be located downstream from the initiation site and can be at a considerable distance from that site.

An enhancer-promoter used in a vector construct of the present invention can be any enhancer-promoter that drives expression in a cell to be transfected. By employing an enhancer-promoter with well-known properties, the level and pattern of gene product expression can be optimized.

A coding sequence of an expression vector is operatively linked to a transcription termination region. RNA polymerase transcribes an encoding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (mRNA). Transcription-terminating regions are well known in the art. A preferred transcription-terminating region used in a vector construct of the present invention comprises a polyadenylation signal of SV40 or the protamine gene. The bGH polyadenylation signal is also suitable for use.

An expression vector comprises a polynucleotide that encodes an Alloiococcus otitidis polypeptide. Such a polypeptide is meant to include a sequence of nucleotide bases encoding an Alloiococcus otitidis polypeptide sufficient in length to distinguish the segment from a polynucleotide segment encoding a non-Alloiococcus otitidis polypeptide. A polypeptide of the invention can also encode biologically functional polypeptides or peptides, which have variant amino acid sequences, such as with changes selected, based on considerations such as the relative hydropathic score of the amino acids being exchanged. These variant sequences are those isolated from natural sources or induced in the sequences disclosed herein using a mutagenic procedure such as site-directed mutagenesis.

Preferably, an expression vector of the present invention comprises a polynucleotide that encodes a polypeptide comprising the amino acid residue sequence of one of even numbered sequences set forth in SEQ ID NO: 2 through SEQ ID NO:.6650 An expression vector can include an Alloiococcus otitidis polypeptide coding region itself of any of the Alloiococcus otitidis polypeptides noted above or it can contain coding regions bearing selected alterations or modifications in the basic coding region of such an Alloiococcus otitidis polypeptide. Alternatively, such vectors or fragments can encode larger polypeptides or polypeptides which nevertheless include the basic coding region. In any event, it should be appreciated that due to codon redundancy as well as biological functional equivalence, this aspect of the invention is not limited to the particular DNA molecules corresponding to the polypeptide sequences noted above.

Exemplary vectors include the mammalian expression vectors of the pCMV family including pCMV6b and pCMV6c (Chiron Corp., Emeryville Calif.). In certain cases, and specifically in the case of these individual mammalian expression vectors, the resulting constructs can require co-transfection with a vector containing a selectable marker such as pSV2neo. Via co-transfection into a dihydrofolate reductase-deficient Chinese hamster ovary cell line, such as DG44, clones expressing Alloiococcus otitidis polypeptides by virtue of DNA incorporated into such expression vectors can be detected.

A DNA molecule of the present invention can be incorporated into a vector by a number of techniques that are well known in the art. For instance, the vector pUC18 has been demonstrated to be of particular value in cloning and expression of genes. Likewise, the related vectors M13mp18 and M13mp19 can also be used in certain embodiments of the invention, in particular, in performing dideoxy sequencing.

An expression vector of the present invention is useful both as a means for preparing quantities of the Alloiococcus otitidis polypeptide-encoding DNA itself, and as a means for preparing the encoded polypeptide and peptides. It is contemplated that where Alloiococcus otitidis polypeptides of the invention are made by recombinant means, one can employ either prokaryotic or eukaryotic expression vectors as shuttle systems. In another aspect, the recombinant host cells of the present invention are prokaryotic host cells. Preferably, the recombinant host cells of the invention are bacterial cells of the DH5α strain of Escherichia coli. In general, prokaryotes are preferred for the initial cloning of DNA sequences and constructing the vectors useful in the invention. For example, Escherichia coli K12 strains can be particularly useful. Other microbial strains that can be used include Escherichia coli B, Escherichia coli W3110 (ATCC No. 273325) and Escherichia. coliₓ1976 (ATCC No. 31537). Bacilli such as Bacillus subtilis, or other enterobacteriaceae such as Salmonella typhimurium or Serratia marcesans, and various Pseudomonas species can be used. These examples are, of course, intended to be illustrative rather than limiting.

In general, plasmid vectors containing replicon and control sequences that are derived from species compatible with the host cell are used in connection with these hosts. The vector ordinarily carries a replication site, as well as marking sequences that are capable of providing phenotypic selection in transformed cells. For example, Escherichia coli is transformed using pBR322, a plasmid derived from an Escherichia coli species (Bolivar, et al. 1977). pBR322 contains genes for ampicillin and tetracycline resistance and thus provides an easy means for identifying transformed cells. The pBR322 plasmid, or other microbial plasmid or phage, must also contain, or be modified to contain, promoters, which can be used by the microbial organism for expression of its own polypeptides.

Those promoters most commonly used in recombinant DNA construction include the β-lactamase (penicillinase) and lactose promoter systems (Chang, et al. 1978; Itakura, et al. 1977, Goeddel, et al. 1979; Goeddel, et al. 1980) and a tryptophan (TRP) promoter system (EP 0036776; Siebwenlist et al. 1980). While these are the most commonly used, other microbial promoters have been discovered and utilized, and details concerning their nucleotide sequences have been published, enabling a skilled worker to introduce functional promoters into plasmid vectors (Siebwenlist, et al. 1980).

In addition to prokaryotes, eukaryotic microbes such as yeast can also be used. Saccharomyces cerevisiase or common baker's yeast is the most commonly used among eukaryotic microorganisms, although a number of other strains are commonly available. For expression in Saccharomyces, the plasmid YRp7, for example, is commonly used (Stinchcomb, et al. 1979; Kingsman, et al. 1979; Tschemper, et al. 1980). This plasmid already contains the trpl gene that provides a selection marker for a mutant strain of yeast lacking the ability to grow in tryptophan, for example ATCC No. 44076 or PEP4-1 (Jones, 1977). The presence of the trpl lesion as a characteristic of the yeast host cell genome then provides an effective environment for detecting transformation by growth in the absence of tryptophan.

Suitable promoter sequences in yeast vectors include the promoters for 3-phosphoglycerate kinase (PGK) (Hitzeman, et al. 1980) or other glycolytic enzymes such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also introduced into the expression vector downstream from the sequences to be expressed to provide polyadenylation of the mRNA and termination. Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin of replication, and termination sequences is suitable.

In addition to microorganisms, cultures of cells derived from multicellular organisms can also be used as hosts. In principle, any such cell culture is workable, whether from vertebrate or invertebrate culture. However, interest has been greatest in vertebrate cells, and propagation of vertebrate cells in culture (tissue culture) has become a routine procedure in recent years. Examples of such useful host cell lines are AtT-20, VERO, HeLa, NSO, PER C6, Chinese hamster ovary (CHO) cell lines, W138, BHK, COSM6, COS-7, 293 and MDCK cell lines. Expression vectors for such cells ordinarily include (if necessary) an origin of replication, a promoter located upstream of the gene to be expressed, along with any necessary ribosome binding sites, RNA splice sites, polyadenylation site, and transcriptional terminator sequences.

Where expression of recombinant Alloiococcus otitidis polypeptides is desired and a eukaryotic host is contemplated, it is most desirable to employ a vector, such as a plasmid, that incorporates a eukaryotic origin of replication. Additionally, for the purposes of expression in eukaryotic systems, one desires to position the Alloiococcus otitidis encoding sequence adjacent to and under the control of an effective eukaryotic promoter such as promoters used in combination with Chinese hamster ovary cells (CHO). To bring a coding sequence under control of a promoter, whether it is eukaryotic or prokaryotic, what is generally needed is to position the 5′ end of the translation initiation site of the proper translational reading frame of the polypeptide between about 1 and about 50 nucleotides 3′ of (downstream) the promoter chosen. Furthermore, where eukaryotic expression is anticipated, one would typically desire to incorporate an appropriate polyadenylation site into the transcriptional unit that includes the Alloiococcus otitidis polypeptide.

A transfected cell can be prokaryotic or eukaryotc. Preferably, the host cells of the invention are prokaryotic host cells. Where it is of interest to produce an Alloiococcus otitidis polypeptide, cultured prokaryotic host cells are of particular interest.

In yet another embodiment, the present invention contemplates a process or method of preparing Alloiococcus otitidis polypeptides comprising transfecting, transforming or infecting cells with a polynucleotide that encodes an Alloiococcus otitidis polypeptide to produce transformed host cells; and maintaining the transformed host cells under biological conditions sufficient for expression of the polypeptide. Preferably, the transformed host cells are prokaryotic cells. Alternatively, the host cells are eukaryotic cells. More preferably, the prokaryotic cells are bacterial cells of the DH5α strain of Escherichia coli. Even more preferably, the polynucleotide transfected into the transformed cells comprises the nucleic acid sequence of one of the odd numbered sequences set forth in SEQ ID NO: 1 through SEQ ID NO: 6649. Additionally, transfection is accomplished using an expression vector disclosed above. A host cell used in the process is capable of expressing a functional, recombinant Alloiococcus otitidis polypeptide.

Following transfection, the cell is maintained under culture conditions for a period of time sufficient for expression of an Alloiococcus otitidis polypeptide. Culture conditions are well known in the art and include ionic composition and concentration, temperature, pH and the like. Typically, transfected cells are maintained under culture conditions in a culture medium. Suitable media for various cell types are well known in the art. in a preferred embodiment, temperature is from about 20° C. to about 50° C., more preferably from about 30° C. to about 40° C. and, even more preferably about 37° C.

The pH is preferably from about a value of 6.0 to a value of about 8.0, more preferably from about a value of about 6.8 to a value of about 7.8 and, most preferably about 7.4. Osmolality is preferably from about 200 milliosmols per liter (mosm/L) to about 400 mosm/l and, more preferably from about 290 mosm/L to about 310 mosm/L. Other biological conditions needed for transfection and expression of an encoded protein are well known in the art.

Transfected cells are maintained for a period of time sufficient for expression of an Alloiococcus otitidis polypeptide. A suitable time depends inter alla upon the cell type used and is readily determinable by a skilled artisan. Typically, maintenance time is from about 2 to about 14 days.

Recombinant Alloiococcus otitidis polypeptide is recovered or collected either from the transfected cells or the medium in which those cells are cultured. Recovery comprises isolating and purifying the Alloiococcus otitidis polypeptide. Isolation and purification techniques for polypeptides are well known in the art and include such procedures as precipitation, filtration, chromatography, electrophoresis and the like.

### F. ANTIBODIES IMMUNOREACTIVE WITH ALLOIOCOCCUS OTITIDIS POLYPEPTIDES

In still another embodiment, the present invention provides antibodies immunoreactive with Alloiococcus otitidis polypeptides. Preferably, the antibodies of the invention are monoclonal antibodies. Additionally, the Alloiococcus otitidis polypeptides comprise the amino acid residue sequence of one of the even numbered sequences set forth in SEQ ID NO: 2 through SEQ ID NO: 6650. Means for preparing and characterizing antibodies are well known in the art (see, e.g., Antibodies “A Laboratory. Manual”, E. Howell and D. Lane, Cold Spring Harbor Laboratory, 1988). Polyclonal antisera is obtained by bleeding an immunized animal into a glass or plastic container, incubating the blood at 25° C. for one hour, followed by incubating at 4° C. for 2-18 hours. The serum is then recovered by centrifugation.

Briefly, a polyclonal antibody is prepared by immunizing the animal with an immunogen comprising a polypeptide or polynucleotide of the present invention, and collecting antisera from that immunized animal. A wide range of animal species can be used for the production of antisera. Typically an animal used for production of anti-antisera is a rabbit, a mouse, a rat, a hamster or a guinea pig. Because of the relatively large blood volume of rabbits, a rabbit is a preferred choice for production of polyclonal antibodies.

As is well known in the art, a given polypeptide or polynucleotide may vary in its immunogenicity. It is often necessary therefore to couple the immunogen (e.g., a polypeptide or polynucleotide) of the present invention with a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers.

Means for conjugating a polypeptide or a polynucleotide to a carrier protein are well known in the art and include glutaraldehyde, m-maleimidobencoyl-N-hydroxysuccinimide ester, carbodiimide and bis-biazotized benzidine.

The amount of immunogen used for the production of polyclonal antibodies varies inter alia, upon the nature of the immunogen as well as the animal used for immunization. A variety of routes can be used to administer the immunogen (subcutaneous, intramuscular, intradermal, intravenous and intraperitoneal). The production of polyclonal antibodies is monitored by sampling blood from the immunized animal at various points following immunization. When a desired level of immunogenicity is obtained, the immunized animal can be bled and the serum isolated and stored.

In another aspect, the present invention contemplates a process of producing an antibody immunoreactive with an Alloiococcus otitidis polypeptide comprising the steps of (a) transfecting recombinant host cells with a polynucleotide that encodes an Alloiococcus otitidis polypeptide; (b) culturing the host cells under conditions sufficient for expression of the polypeptide; (c) recovering the polypeptides; and (d) preparing the antibodies to the polypeptides. Preferably, the host cell is transfected with the polynucleotide of one of the odd numbered sequences set forth in SEQ ID NO: 1 through SEQ ID NO: 6649. Even more preferably, the present invention provides antibodies prepared according to the process described above.

A monoclonal antibody of the present invention is readily prepared through use of well-known techniques such as those exemplified in U.S. Pat. No. 4,196,265, herein incorporated by reference. Typically, the technique involves first immunizing a suitable animal with a selected antigen (e.g., a polypeptide or polynucleotide of the present invention) in a manner sufficient to provide an immune response. Rodents such as mice and rats are preferred animals. Spleen cells from the immunized animal are then fused with cells of an immortal myeloma cell. Where the immunized animal is a mouse, a preferred myeloma cell is a murine NS-1 myeloma cell.

The fused spleen/myeloma cells are cultured in a selective medium to select fused spleen/myeloma cells from the parental cells. Fused cells are separated from the mixture of non-fused parental cells, e.g., by the addition of agents that block the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides. Where azaserine is used, the media is supplemented with hypoxanthine.

This culturing provides a population of hybridomas from which specific hybridomas are selected. Typically, selection of hybridomas is performed by culturing the cells by single-clone dilution in microtiter plates, followed by testing the individual clonal supernatants for reactivity with an antigen-polypeptide. The selected clones are then propagated indefinitely to provide the monoclonal antibody.

By way of specific example, to produce an antibody of the present invention, mice are injected intraperitoneally with between about 1-200 μg of an antigen comprising a polypeptide of the present invention. B lymphocyte cells are stimulated to grow by injecting the antigen in association with an adjuvant such as complete Freund's adjuvant (CFA; a non-specific stimulator of the immune response containing killed Mycobacterium tuberculosis). At some time (e.g., at least two weeks) after the first injection, mice are boosted by injection with a second dose of the antigen mixed with incomplete Freund's adjuvant (IFA; lacks the killed mycobacterium of CFA).

A few weeks after the second injection, mice are tail bled and the sera titered by immunoprecipitation against radiolabeled antigen. Preferably, the process of boosting and titering is repeated until a suitable titer is achieved. The spleen of the mouse with the highest titer is removed and the spleen lymphocytes are obtained by homogenizing the spleen with a syringe. Typically, a spleen from an immunized mouse contains approximately 5×10⁷ to 2×10⁸ lymphocytes.

Mutant lymphocyte cells known as myeloma cells are obtained from laboratory animals in which such cells have been induced to grow by a variety of well-known methods. Myeloma cells lack the salvage pathway of nucleotide biosynthesis. Because myeloma cells are tumor cells, they can be propagated indefinitely in tissue culture, and are thus denominated immortal. Numerous cultured cell lines of myeloma cells from mice and rats, such as murine NS-1 myeloma cells, have been established.

Myeloma cells are combined under conditions appropriate to foster fusion with the normal antibody-producing cells from the spleen of the mouse or rat injected with the antigen/polypeptide of the present invention. Fusion conditions include, for example, the presence of polyethylene glycol. The resulting fused cells are hybridoma cells. Like myeloma cells, hybridoma cells grow indefinitely in culture.

Hybridoma cells are separated from unfused myeloma cells by culturing in a selection medium such as HAT media (hypoxanthine, aminopterin, thymidine). Unfused myeloma cells lack the enzymes necessary to synthesize nucleotides from the salvage pathway because they are killed in the presence of aminopterin, methotrexate, or azaserine. Unfused lymphocytes also do not continue to grow in tissue culture. Thus, only cells that have successfully fused (hybridoma cells) can grow in the selection media.

Each of the surviving hybridoma cells produces a single antibody. These cells are then screened for the production of the specific antibody immunoreactive with an antigen/polypeptide of the present invention. Single cell hybridomas are isolated by limiting dilutions of the hybridomas. The hybridomas are serially diluted many times and, after the dilutions are allowed to grow, the supematant is tested for the presence of the monoclonal antibody. The clones producing that antibody are then cultured in large amounts to produce an antibody of the present invention in convenient quantity.

By use of a monoclonal antibody of the present invention, specific polypeptides and polynucleotide of the invention are identified as antigens. Once identified, those polypeptides and polynucleotide are isolated and purified by techniques such as antibody-affinity chromatography. In antibody-affinity chromatography, a monoclonal antibody is bound to a solid substrate and exposed to a solution containing the desired antigen. The antigen is removed from the solution through an immunospecific reaction with the bound antibody. The polypeptide or polynucleotide is then easily removed from the substrate and purified.

Additionally, examples of methods and reagents particularly amenable for use in generating and screening an antibody display library can be found in, for example, U.S. Pat. No. 5,223,409; WO 92/18619; WO 91/17271; WO 92/20791; WO 92/15679; WO 93/01288; WO 92/01047; WO 92/09690; WO 90/02809, which are incorporated herein in their entirety by reference.

Additionally, recombinant anti-Alloiococcus otitidis antibodies, such as chimeric and humanized monoclonal antibodies, comprising both human and non-human fragments, which are made using standard recombinant DNA techniques, are within the scope of the invention. Such chimeric and humanized monoclonal antibodies are produced by recombinant DNA techniques known in the art, for example using methods described in PCT/US86/02269; EP 184,187; EP 171,496; EP 173,494; WO 86/01533; U.S. Pat. No. 4,816,567; and EP 125,023.

An anti-Alloiococcus otitidis antibody (e.g., monoclonal antibody) is used to isolate Alloiococcus otitidis polypeptides by standard techniques, such as affinity chromatography or immunoprecipitation. An anti-Alloiococcus otitidis antibody facilitates the purification of a natural Alloiococcus otitidis polypeptide from cells and recombinantly produced Alloiococcus otitidis polypeptides expressed in host cells. Moreover, an anti-Alloiococcus otitidis antibody is used to detect Alloiococcus otitidis polypeptide (e.g., in a cellular lysate or cell supernatant) in order to evaluate the abundance of the Alloiococcus otitidis polypeptide. The detection of circulating fragments of an Alloiococcus otitidis polypeptide is used to identify Alloiococcus otitidis polypeptide turnover in a subject. Anti-Alloiococcus otitidis antibodies are used diagnostically to monitor protein levels in issue as part of a clinical testing procedure, e.g., to, for example, determine the efficacy of a given treatment regimen. Detection is facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylarnine fluorescein, dansyl chloride or phycoerythrin; an example of a luminescent material includes luminol; examples of bioluminescent materials include luciferase, luciferin, and acquorin, and examples of suitable radioactive material include ¹²⁵I, ¹³¹I, ¹⁵S or ³H.

### G. PHARMACEUTICAL AND IMMUNOGENIC COMPOSITIONS

In certain embodiments, the present invention provides pharmaceutical and immunogenic compositions comprising Alloiococcus otitidis polypeptides, and physiologically acceptable carriers. More preferably, the pharmaceutical and immunogenic compositions comprise Alloiococcus otitidis polypeptides comprising the amino acid residue sequence of one or more of the even numbered sequences set forth in SEQ ID NO: 2 through SEQ ID NO: 6650. In other embodiments, the pharmaceutical and immunogenic compositions of the invention comprise polynucleotides that encode Alloiococcus otitidis polypeptides, such as the polynucleotides comprising the nucleotide sequence of one of odd numbered sequences set forth in SEQ ID NO: 1 through SEQ ID NO: 6649, and physiologically acceptable carriers. Various tests may be used to assess the in vitro immunogenicity of the polypeptides of the invention. For example, an in vitro opsonic assay is conducted by incubating together a mixture of Alloiococcus otitidis cells, heat inactivated human serum containing specific antibodies to the polypeptide in question, and an exogenous complement source. Opsonophagocytosis proceeds during incubation of freshly isolated human polymorphonuclear cells (PMN) and the antibody/complement/Alloiococcal cell mixture. Bacterial cells that are coated with antibody and complement are killed upon opsonophagocytosis. Colony forming units (cfu) of surviving bacteria that escape from opsonophagocytosis are determined by plating the assay mixture. Titers are reported as the reciprocal of the highest dilution that gives ≧50% bacterial killing, as determined comparison to assay controls. The method described above is a modification of Gray's method (Gray, B. M., 1990).

A whole Alloiococcus otitidis cell ELISA assay is also used to assess in vitro immunogenicity and surface exposure of the polypeptide antigen, wherein the bacterial strain of interest (Alloiococcus otitidis) is coated onto a plate, such as 96 well plate, and test sera from an immunized animal is reacted with bacterial cells. If any antibody, specific for the test polypeptide antigen, is reactive with surface exposed epitope of the polypeptide antigen, it can be detected by standard methods known to one of skill in the art.

Any polypeptide demonstrating the desired in vitro activity is then tested in an in vivo animal challenge model. In certain embodiments, immunogenic compositions are used in the immunization of an animal (e.g., a mouse) by methods and routes of immunization known to those of skill in the art (e.g., intranasal, parenteral, intraperitoneal, intravenous, subcutaneous, etc.). Following immunization of the animal with a particular Alloiococcus otitidis immunogenic composition, the animal is challenged with Alloiococcus otitidis and assayed for resistance to Alloiococcus otitidis infection.

The Alloiococcus otitidis polynucleotides and polypeptides, and modulators of Alloiococcus otitidis polynucleotides and polypeptides, and anti-Alloiococcus otitidis antibodies (also referred to herein as “active compounds”) of the invention can be incorporated into pharmaceutical and immunogenic compositions suitable for administration to a subject, e.g., a human. Such compositions typically comprise the nucleic acid molecule, protein, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical or immunogenic composition of the invention is formulated to be compatible with its intended route of administration. Examples of routes of administration include parenteral, (e.g., intravenous, intradermal, subcutaneous, intramuscular, and intraperitoneal), mucosal (e.g., oral, rectal, buccal, vaginal, intranasal, inhalation), and transdermal (topical). Solutions or suspensions used for parenteral, intradermal, or subcutaneous application can include the following components: a sterile diluents such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™(BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier is a solvent or dispersion medium containing, for example, water, ethanol, polyol (for example, glycerol, propylene glycol, and liquid polyetheylene glycol, and the like), and suitable mixtures thereof. The proper fluidity is maintained, for example, by the use of a coating such as lecithin, by the maintenance of the required particle size in the case of dispersion and by the use of surfactants. Prevention of the action of microorganisms is achieved by various antibacterial and antifungal agents, for example, parabens, chlorobutanol, phenol, ascorbic acid,. thimerosal, and the like. In many cases, it is preferable to include isotonic agents, for example, sugars, polyalcohols such as manitol, sorbitol, and sodium chloride in the composition. Prolonged absorption of the injectable compositions is brought about by including in the composition an agent which delays absorption, for example, aluminum monostearate and gelatin.

Sterile injectable solutions are prepared by incorporating the active compound (e.g., an Alloiococcus otitidis polypeptide or anti-Alloiococcus otitidis antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound is incorporated with excipients and used in the form of tablets, troches, or capsules. Oral compositions are also prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer. Systemic administration can also be by mucosal or transdermal means. For mucosal or transdermal administration, penetrants appropriate to the barrier to be permeated are used in the formulation. Such penetrants are generally known in the art, and include, for example, for mucosal administration, detergents, bile salts, and fusidic acid derivatives. Mucosal administration is accomplished through the use of nasal sprays or suppositories. For transdermal administration, the active compounds are formulated into ointments, salves, gels, or creams as generally known in the art.

The compounds is also prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems.

Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials are also obtained commercially from Alza Corporation (Palo Alto, Calif.) and Nova Pharmaceuticals, Inc. (Clifton, N.J.) Liposomal suspensions (including liposomes targeted to infected cells with monoclonal antibodies to viral antigens) are also used as pharmaceutically acceptable carriers. These are prepared according to methods known to those skilled in the art, for example, as described in U.S. Pat. No. 4,522,811, which is incorporated herein by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Combination immunogenic compositions are provided by including two or more of the polypeptides of this invention. Multivalent immunogenic compositions directed against various bacteria responsible for causing otitis media comprise one or more of the polypeptides of this invention together with one or more known nontypable Haemophilus influenzae polypeptides, including, but not limited to, the P2, P4, P5, P6 and PCP proteins, and/or one or more known Moraxella catarrhalis polypeptides, including, but not limited to, the UspA1, UspA2, B1, C/D, E and 74 kDa proteins, and/or one or more known Streptococcus pneumoniae polypeptides and polysaccharide-protein conjugates, including, but not limited to, the currently available 23-valent pneumococcal capsular polysaccharide vaccine and the 7-valent pneumococcal polysaccharide-protein conjugate vaccine. One particularly preferred multivalent immunogenic composition comprises one or more of Alloiococcus otitidis polypeptides of the invention together with P4, P6, and UspA2 polypeptides.

The nucleic acid molecules of the invention are inserted into a variety of vectors and expression systems. A great variety of expression systems are used. Such systems include, among others, chromosomal, episomal and virus-derived systems, e.g., vectors derived from bacterial plasmids, attenuated bacteria such as Salmonella (U.S. Pat. No. 4,837,151) from bacteriophage, from transposons, from yeast episomes, from insertion elements, from yeast chromosomal elements, from viruses such as vaccinia and other poxviruses, sindbis, adenovirus, baculoviruses, papova viruses, such as SV40, fowl pox viruses, pseudorabies viruses and retroviruses, alphaviruses such as Venezuelan equine encephalitis virus (U.S. Pat. No. 5,643,576), nonsegmented negative-stranded RNA viruses such as vesicular stomatitis virus (U.S. Pat. No. 6,168,943), and vectors derived from combinations thereof, such as those derived from plasmid and bacteriophage genetic elements, such as cosmids and phagemids. The expression systems should include control regions that regulate as well as engender expression, such as promoters and other regulatory elements (such as a polyadenylation signal). Generally, any system or vector suitable to maintain, propagate or express polynucleotides to produce a polypeptide in a host may be used. The appropriate nucleotide sequence may be inserted into an expression system by any of a variety of well-known and routine techniques, such as, for example, those set forth in Sambrook et al., “Molecular Cloning: A Laboratory Manual” 2nd, ed, Cold Spring Harbor Laboratory, Cold Spring Harbor Laboratory Press, Cold Spring Harbor, N.Y., 1989.

The nucleic acid molecules of the invention inserted into vectors as described above are also used to deliver the nucleic acid molecules to a subject by, for example, intravenous injection, local administration (see U.S. Pat. No. 5,328,470) or by stereotactic injection (see e.g., Chen et al., 1994). The pharmaceutical preparation for the administration of the nucleic acid molecules of the invention include, in addition to the nucleic molecules in an appropriate vector, an acceptable diluent, or can comprise a slow release matrix in which the nucleic acid molecule is embedded. Alternatively, where the complete nucleic acid moleculer is produced intact from recombinant cells, e.g. retroviral vectors, the pharmaceutical preparation includes one or more cells which produce the gene delivery system. The pharmaceutical compositions are included in a container, pack, or dispenser together with instructions for administration.

A pharmaceutically acceptable vehicle is understood to designate a compound or a combination of compounds entering into a pharmaceutical or immunogenic composition which does not cause side effects and which makes it possible, for example, to facilitate the administration of the active compound, to increase its life and/or its efficacy in the body, to increase its solubility in solution or alternatively to enhance its preservation. These pharmaceutically acceptable vehicles are well known and will be adapted by persons skilled in the art according to the nature and the mode of administration of the active compound chosen.

Immunogenic compositions may comprise appropriate adjuvants to modulate, shift and/or boost the immune response of the recipient to the immunogen. Such adjuvants are known to persons skilled in the art, such as those described in section H below.

A composition of the present invention is typically administered parenterally in dosage unit formulations containing standard, well-known nontoxic physiologically acceptable carriers, adjuvants, and vehicles as desired. The term parenteral as used herein includes intravenous, intramuscular, subcutaneous, intradermal, intraarterial injection, or infusion techniques, as well as other conventional techniques or routes described above.

Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol.

Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil is employed including synthetic mono- or di-glycerides. In addition, fatty acids such as oleic acid find use in the preparation of injectables.

Preferred carriers include neutral saline solutions buffered with phosphate, lactate, Tris, and the like. When administering viral vectors, the vector is purified sufficiently to render it essentially free of undesirable contaminants, such as defective interfering adenovirus particles or endotoxins and other pyrogens such that it does not cause any untoward reactions in the individual receiving the vector construct. A preferred means of purifying the vector involves the use of buoyant density gradients, such as cesium chloride gradient centrifugation.

A carrier can also be a liposome. Means for using liposomes as delivery vehicles are well known in the art (see, e.g. Gabizon et al., 1990; and Ranade, V. V., 1989).

The immunogenic compositions of this invention also comprise a polynucleotide sequence of this invention operatively associated with a regulatory sequence that controls gene expression. The polynucleotide sequence of interest is engineered into an expression vector, such as a plasmid, under the control of regulatory elements which will promote expression of the DNA, that is, promoter and/or enhancer elements. In a preferred embodiment, the human cytomegalovirus immediate-early promoter/enhancer is used (U.S. Pat. No. 5,168,062). The promoter may be cell specific and permit increased expression of the polynucleotide only in predetermined cells.

The polynucleotide is introduced directly into the host either as “naked” DNA (U.S. Pat. No. 5,580,859) or formulated in compositions with agents which facilitate immunization, such as bupivicaine and other local anesthetics (U.S. Pat. No. 6,127,170).

In this polynucleotide immunization procedure, the polypeptides of the invention are expressed on a transient basis in vivo; no genetic material is inserted or integrated into the chromosomes of the host. This procedure is to be distinguished from gene therapy, where the goal is to insert or integrate the genetic material of interest into the chromosome. An assay is used to confirm that the polynucleotides administered by immunization do not rise to a transformed phenotype in the host (U.S. Pat. No. 6,168,918).

### H. USES AND METHODS OF THE INVENTION

The Alloiococcus otitidis polynucleotides, polypeptides, polypeptide homologues, modulators, adjuvants, and antibodies described in this invention are used in methods of treatment, diagnostic assays particularly in disease identification, drug screening assays and monitoring of effects during clinical trials. The isolated polynucleotides of the invention are used to express Alloiococcus otitidis polypeptides (e.g., via a recombinant expression vector in a host cell or in gene therapy applications) and to detect Alloiococcus otitidis mRNA (e.g., in a biological sample). Moreover, the ant-Alloiococcus otitidis antibodies of the invention are used to detect and isolate an Alloiococcus otitidis polypeptide, particularly fragments of an Alloiococcus otitidis polypeptides present in a biological sample, and to modulate Alloiococcus otitidis polypeptide activity.

The invention provides immunogenic compositions comprising polypeptides having an amino acid sequence chosen from one or more of the even numbered sequences set forth in SEQ ID NO: 2 through SEQ ID NO: 6650, a biological equivalent thereof or a fragment thereof. The immunogenic composition may further comprise a pharmaceutically acceptable carrier, as outlined in section G.

In certain preferred embodiments, the immunogenic composition will comprise one or more adjuvants. As defined herein, an “adjuvant” is a substance that serves to enhance the immunogenicity of an “antigen” or the immunogenic compositions comprising a polypeptide antigens having an amino acid sequence chosen from one or more of even numbered sequences set forth in SEQ ID NO: 2 through SEQ ID NO: 6650. Thus, adjuvants are often given to boost the immune response and are well known to the skilled artisan. Preferred adjuvants to enhance effectiveness of the composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59 (PCT Publ. No. WO90/14837), containing 5% Squalene, 0.5% Tween 80, and 0.5% Span 85 (optionally containing various amounts of MTP-PE (see below, although not required) formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi™ adjuvant system (RAS), (Corixa, Hamilton, Mont.) containing 2% Squalene, 0.2% Tween 80, and one or more bacterial cell wall components from the group consisting of 3-O-deacylated monophosphorylipid A (MPL™) described in U.S. Pat. No. 4,912,094 (Corixa), trehalose dimycolate (TDM), and cell wall skeleton (CWS), preferably MPL+CWS (Detox™), (3) saponin adjuvants, such as Quil A or STIMULON™ QS-21 (Antigenics, Framingham, Mass.) (U.S. Pat. No. 5,057,540) may be used or particles generated therefrom such as ISCOMs (immunostimulating complexes); (4) bacterial lipopolysaccharides, synthetic lipid A analogs such as aminoalkyl glucosamine phosphate compounds (AGP), or derivatives or analogs thereof, which are available from Corixa, and which are described in U.S. Pat. No. 6,113,918; one such AGP is 2-[(R)-3-Tetradecanoyloxytetradecanoylamino]ethyl 2-Deoxy-4-O-phosphono-3-O-[(R)-3-tetradecanoyoxytetradecanoyl]-2-[(R)-3-tetradecanoyoxytetradecanoylamino]-b-D-glucopyranoside, which is also known as 529 (formerly known as RC529), which is formulated as an aqueous form or as a stable emulsion, synthetic polynucleotides such as oligonucleotides containing CpG motif(s) (U.S. Pat. No. 6,207,646); (5) cytokines, such as interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), tumor necrosis factor (TNF), etc; (6) detoxified mutants of a bacterial ADP-ribosylating toxin such as a cholera toxin (CT) either in a wild-type or mutant form, for example, wherein the glutamic acid at amino acid position is replaced by another amino acid, preferably a histidine, in accordance with published International patent Application number WO 00/18434, a pertussis toxin (PT, or an E. coli heat-labile toxin (LT), particularly LT-K63, LT-R72, CT-S109, PT-K9/G129; see, e.g., WO 93/13302 and WO 92/19265; and (7) other substances that act as immunostimulating agents to enhance the effectiveness of the composition.

As mentioned above, muramyl peptides include, but are not limited to, N-acetyl-muramyl-L-threonyl-D-isoglutamine(thr-MDP), N-acetyl-normuramyl-L-alanine-2-(1′-2′dipalmitoyl-snglycero-3-huydroxyphosphoryloxy)-ethylamine (MTP-PE), etc.

In another embodiment, the invention provides immunogenic compositions comprising a polynucleotide having a nucleotide sequence chosen from one or more of the odd numbered sequences set forth in SEQ ID NO: 1 through SEQ ID NO: 6649, wherein the polynucleotide is comprised in a recombinant expression vector. Preferably the vector is plasmid DNA. The polynucleotide may further comprise heterologous nucleotides, e.g., the polynucleotide is operatively linked to one or more gene expression regulatory elements, and further comprise one or more adjuvants. In a preferred embodiment, the immunogenic polynucleotide composition directs the expression of a neutralizing epitope of Alloiococcus otitidis.

Provided also are methods for immunizing a host against Alloiococcus otitidis infection. In a preferred embodiment, the host is human. Thus, a host or subject is administered an immunogenic amount of an immunogenic composition comprising a polypeptide having an amino acid sequence chosen from one or more of the even numbered sequences set forth in SEQ ID NO: 2 through SEQ ID NO: 6650, a biological equivalent thereof or a fragment thereof and a pharmaceutically acceptable carrier. An immunogenic amount of an immunogenic composition is determined by doing a dose response study in which subjects are immunized with gradually increasing amounts of the immunogenic composition and the immune response analyzed to determine the optimal dosage. Starting points for the study is inferred from immunization data in animal models. The dosage amount can vary depending upon specific conditions of the individual. The amount can be determined in routine trials by means known to those skilled in the art.

An “immunologically effective amount” of the immunogenic composition in an appropriate number of doses is administered to the subject to elicit an immune response. “Immunologically effective amount”, as used herein, means the administration of that amount to a mammalian host (preferably human), either in a single dose or as part of a series of doses, sufficient to at least cause the immune system of the individual treated to generate a response that reduces the clinical impact of the bacterial infection. Protection may be conferred by a single dose of the immunogenic composition, or may require the administration of several doses, in addition to booster doses at later times to maintain protection. This may range from a minimal decrease in bacterial burden to prevention of the infection. Ideally, the treated individuals will not exhibit the more serious clinical manifestations of the Alloiococcus otitidis infection. The dosage amount can vary depending upon specific conditions of the individual, such as age and weight. The amount can be determined in routine trials by means known to those skilled in the art.

### I. DIAGNOSTIC ASSAYS

The invention provides methods for detecting the presence of an Alloiococcus otitidis polypeptide or Alloiococcus otitidis polynucleotide, or fragment thereof, in a biological sample. The method involves contacting the biological sample with a compound or an agent capable of detecting an Alloiococcus otitidis polypeptide or mRNA such that the presence of the Alloiococcus otitidis polypeptide/encoding nucleic acid molecule is detected in the biological sample. A preferred agent for detecting Alloiococcus otitidis mRNA or DNA is a labeled or labelable oligonucleotide probe capable of hybridizing to Alloiococcus otitidis mRNA or DNA. The nucleic acid probe can be, for example, a full-length Alloiococcus otitidis polynucleotide of one of the odd numbered sequences set forth in SEQ ID NO: 1 through SEQ ID NO: 6649, a complement thereof, or a fragment thereof, such as an oligonucleotide of at least 15, 30, 50, 100, 250 or 500 nucleotides in length and sufficient to specifically hybridize under stringent conditions to Alloiococcus otitidis mRNA or DNA. Alternatively, the sample can be contacted with an oligonucleotide primer of an Alloiococcus otitidis polynucleotide of one of the odd numbered sequences set forth in SEQ ID NO: 1 through SEQ ID No: 6649, a complement thereof, or a fragment thereof, in the presence of nucleotides and a polymerase, under conditions permitting primer extension.

A preferred agent for detecting an Alloiococcus otitidis polypeptide is a labeled or labelable antibody capable of binding to an Alloiococcus otitidis polypeptide of one of the even numbered sequences set forth in SEQ. ID. NO.: 2 through SEQ. ID. NO. 6650. Antibodies can be polyclonal, or more preferably, monoclonal. An intact antibody, or a fragment thereof (e.g., Fab or F(ab′)₂) can be used. The term “labeled or labelable,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it are detected with fluorescently labeled streptavidin. The term “biological sample” is intended to include tissues, cells and biological fluids isolated from a subject, as well as tissues, cells and fluids present within a subject. That is, the detection method of the invention can be used to detect Alloiococcus otitidis mRNA, DNA or protein in a biological sample in vitro as well as in vivo. For example, in vitro techniques for detection of Alloiococcus otitidis mRNA include Northern hybridizations and in situ hybridizations. In vitro techniques for detection of Alloiococcus otitidis polypeptide include enzyme linked immunosorbent assays (ELISAs), Western blots, immunoprecipitations and immunofluorescence. Alternatively, Alloiococcus otitidis polypeptides can be detected in vivo in a subject by introducing into the subject a labeled anti-Alloiococcus otitidis antibody. For example, the antibody can be labeled with a radioactive marker whose presence and location in a subject can be detected by standard imaging techniques.

The polynucleotides according to the invention may also be used in analytical DNA chips, which allow sequencing, the study of mutations and of the expression of genes, and which are currently of interest given their very small size and their high capacity in terms of number of analyses.

The principle of the operation of these chips is based on molecular probes, most often oligonucleotides, which are attached onto a miniaturized surface, generally of the order of a few square centimeters. During an analysis, a sample containing fragments of a target nucleic acid to be analyzed, for example DNA or RNA labeled, for example, after amplification, is deposited onto the DNA chip in which the support has been coated beforehand with probes. Bringing the labeled target sequences into contact with the probes leads to the formation, through hybridization, of a duplex according to the rule of pairing defined by J. D.Watson and F.Crick. After a washing step, analysis of the surface of the chip allows the effective hybridizations to be located by means of the signals emitted by the labels tagging the target. A hybridization fingerprint results from this analysis which, by appropriate computer processing, will make it possible to determine information such as the presence of specific fragments in the sample, the determination of sequences and the presence of mutations.

The chip consists of a multitude of molecular probes, precisely organized or arrayed on a solid support whose surface is miniaturized. It is at the center of a system where other elements (imaging system, microcomputer) allow the acquisition and interpretation of a hybridization fingerprint.

The hybridization supports are provided in the form of flat or porous surfaces (pierced with wells) composed of various materials. The choice of a support is determined by its physicochemical properties, or more precisely, by the relationship between the latter and the conditions under which the support will be placed during the synthesis or the attachment of the probes or during the use of the chip. It is therefore necessary, before considering the use of a particular support, to consider characteristics such as its stability to pH, its physical strength, its reactivity and its chemical stability as well as its capacity to nonspecifically bind nucleic acids. Materials such as glass, silicon and polymers are commonly used. Their surface is, in a first step, called “functionalization”, made reactive towards the groups which it is desired to attach thereon. After the functionalization, so-called spacer molecules are grafted onto the activated surface. Used as intermediates between the surface and the probe, these molecules of variable size render unimportant the surface properties of the supports, which often prove to be problematic for the synthesis or the attachment of the probes and for the hybridization.

Among the hybridization supports, there may be mentioned glass which is used, for example, in the method of in situ synthesis of oligonucleotides by photochemical addressing developed by the company Affymetrix (E. L. Sheldon, 1993), the glass surface being activated by silane. Genosensor Consortium (P. Mérel, 1994) also uses glass slides carrying wells 3 mm apart, this support being activated with epoxysilane.

The probes according to the invention may be synthesized directly in situ on the supports of the DNA chips. This in situ synthesis may be carried out by photochemical addressing (developed by the company Affymax (Amsterdam, Holland) and exploited industrially by its subsidiary Affymetrix (United States)) or based on the VLSIPS (very large scale immobilized polymer synthesis) technology (S. P. A. Fodor et al., 1991) which is based on a method of photochemically directed combinatory synthesis and the principle of which combines solid-phase chemistry, the use of photolabile protecting groups and photolithography.

The probes according to the invention may be attached to the DNA chips in various ways such as electrochemical addressing, automated addressing or the use of probe printers (T. Livache et al., 1994; G. Yershov et al., 1996; and J. Derisi et al., 1996)

The revealing of the hybridization between the probes of the invention, deposited or synthesized in situ on the supports of the DNA chips, and the sample to be analyzed, may be determined, for example, by measurement of fluorescent signals, by radioactive counting or by electronic detection.

The use of fluorescent molecules such as fluorescein constitutes the most common method of labeling the samples. It allows direct or indirect revealing of the hybridization and allows the use of various fluorochromes.

Affymetrix currently provides an apparatus or a scanner designed to read its Gene Chip™ chips. It makes it possible to detect the hybridizations by scanning the surface of the chip in confocal microscopy (R. J. Lipshutz et al., 1995).

The nucleotide sequences according to the invention are also used in DNA chips to carry out the analysis of the expression of the Alloiococcus otitidis genes. This analysis of the expression of Alloiococcus otitidis genes is based on the use of chips where probes of the invention, chosen for their specificity to characterize a given gene, are present (D. J. Lockhart et al., 1996; D. D. Shoemaker et al., 1996). For the methods of analysis of gene expression using the DNA chips, reference may, for example, be made to the methods described by D. J. Lockhart et al. (1996) and Sosnowsky et al. (1997) for the synthesis of probes in situ or for the addressing and the attachment of previously synthesized probes. The target sequences to be analyzed are labeled and in general fragmented into sequences of about 50 to 100 nucleotides before being hybridized onto the chip. After washing as described, for example, by D. J. Lockhart et al. (1996) and application of different electric fields (Sosnowsky et al., 1997), the labeled compounds are detected and quantified, the hybridizations being carried out at least in duplicate. Comparative analyses of the signal intensities obtained with respect to the same probe for different samples and/or for different probes with the same sample, determine the differential expression of RNA or of DNA derived from the sample.

The nucleotide sequences according to the invention are, in addition, used in DNA chips where other nucleotide probes specific for other microorganisms are also present, and allow the carrying out of a serial test allowing rapid identification of the presence of a microorganism in a sample.

Accordingly, the subject of the invention is also the nucleotide sequences according to the invention, characterized in that they are immobilized on a support of a DNA chip.

The DNA chips, characterized in that they contain at least one nucleotide sequence according to the invention, immobilized on the support of the said chip, also form part of the invention.

The chips preferably contain several probes or nucleotide sequences of the invention of different length and/or corresponding to different genes so as to identify, with greater certainty, the specificity of the target sequences or the desired mutation in the sample to be analyzed.

Accordingly, the analyses carried out by means of primers and/or probes according to the invention, immobilized on supports such as DNA chips, make it possible, for example, to identify, in samples, mutations linked to variations such as intraspecies variations. These variations may be correlated or associated with pathologies specific to the variant identified and make it possible to select the appropriate treatment.

The invention thus comprises a DNA chip according to the invention, characterized in that it contains, in addition, at least one nucleotide sequence of a microorganism different from Alloiococcus otitidis, immobilized on the support of the said chip; preferably, the different microorganism is chosen from an associated microorganism, a bacterium of the Streptococcus family, and a variant of the species Alloiococcus otitidis.

The principle of the DNA chip as explained above, is also used to produce protein “chips” on which the support has been coated with a polypeptide or an antibody according to the invention, or arrays thereof, in place of the DNA. These protein “chips” make it possible, for example, to analyze the biomolecular interactions (BIA) induced by the affinity capture of target analytes onto a support coated, for example, with proteins, by surface plasma resonance (SPR). Reference may be made, for example, to the techniques for coupling proteins onto a solid support which are described in EP 524 800 or to the methods describing the use of biosensor-type protein chips such as the BIAcore-type technique (Pharmacia) (Arlinghaus et al., 1997 and Krone et al., 1997). These polypeptides or antibodies according to the invention, capable of specifically binding antibodies or polypeptides derived from the sample to be analyzed, are thus used in protein chips for the detection and/or the identification of proteins in samples. The said protein chips may in particular be used for infectious diagnosis and preferably contain, per chip, several polypeptides and/or antibodies of the invention of different specificity, and/or polypeptides and/or antibodies capable of recognizing microorganisms different from Alloiococcus otitidis.

Accordingly, the present invention also encompasses polypeptides and the antibodies according to the invention, characterized in that they are immobilized on a support, in particular, on a protein chip.

The protein chips, characterized in that they contain at least one polypeptide or one antibody according to the invention immobilized on the support of the said chip, also form part of the invention.

The invention comprises, in addition, a protein chip according to the invention, characterized in that it contains, in addition, at least one polypeptide of a microorganism different from Alloiococcus otitidis or at least one antibody directed against a compound of a microorganism different from Alloiococcus otitidis, immobilized on the support of the chip.

The invention also relates to a kit or set for the detection and/or the identification of bacteria belonging to the species Alloiococcus otitidis or to an associated microorganism, or for the detection and/or the identification of a microorganism characterized in that it comprises a protein chip according to the invention.

The present invention also provides a method for the detection and/or the identification of bacteria belonging to the species Alloiococcus otitidis or to an associated microorganism in a biological sample, characterized in that it uses a nucleotide sequence according to the invention.

The invention also encompasses kits for detecting the presence of an Alloiococcus otitidis polypeptide in a biological sample. For example, the kit comprises reagents such as a labeled or labelable compound or agent capable of detecting Alloiococcus otitidis polypeptide or mRNA in a biological sample; means for determining the amount of Alloiococcus otitidis polypeptide in the sample; and means for comparing the amount of Alloiococcus otitidis polypeptide in the sample with a standard. The compound or agent is packaged in a suitable container. The kit further comprises instructions for using the kit to detect Alloiococcus otitidis mRNA or protein.

In certain embodiments, detection involves the use of a probe/primer in a polymerase chain reaction (PCR) (see, e.g. U.S. Pat. No. 4,683,195 and U.S. Pat. No. 4,683,202), such as anchor PCR or RACE PCR, or, alternatively, in a ligation chain reaction (LCR). This method includes the steps of collecting a sample of cells from a patient, isolating nucleic acid (e.g., genomic, mRNA or both) from the cells of the sample, contacting the nucleic acid sample with one or more primers which specifically hybridize to an Alloiococcus otitidis polynucleotide under conditions such that hybridization and amplification of the Alloiococcus otitidis-polynucleotide (if present) occurs, and detecting the presence or absence of an amplification product, or detecting the size of the amplification product and comparing the length to a control sample.

### J. TRANSGENIC ANIMALS

It is contemplated that in some instances the genome of a transgenic animal of the present invention will have been altered through the stable introduction of one or more of the Alloiococcus otitidis polynucleotide compositions described herein, either native, synthetically modified or mutated. As described herein, a “transgenic animal” refers to any non-human animal, preferably a non-human mammal (e.g. mouse, rat, rabbit, squirrel, hamster, rabbits, guinea pigs, pigs, micro-pigs, prairie, baboons, squirrel monkeys and chimpanzees, etc), bird or an amphibian, in which one or more cells contain a heterologous nucleic acid sequence introduced by way of human intervention, such as by transgenic techniques well known in the art. The nucleic acid is introduced into the cell, directly or indirectly, by introduction into a precursor of the cell, by way of deliberate genetic manipulation, such as by microinjection or by infection with a recombinant virus. The term genetic manipulation does not include classical crossbreeding, or in vitro fertilization, but rather is directed to the introduction of a recombinant DNA molecule. This molecule may be integrated within a chromosome, or it may be extrachromosomally replicating DNA.

The host cells of the invention are also used to produce non-human transgenic animals. The non-human transgenic animals are used in screening assays designed to identify infections or compounds, e.g., drugs, pharmaceuticals, etc., which are capable of ameliorating Alloiococcus otitidis symptoms or infections. For example, in one embodiment, a host cell of the invention is a fertilized oocyte or an embryonic stem cell into which an Alloiococcus otitidis polypeptide-coding sequence has been introduced. Such host cells are then used to create non-human transgenic animals in which exogenous Alloiococcus otitidis gene sequences have been introduced into their genome or homologous recombinant animals in which endogenous Alloiococcus otitidis gene sequences have been altered. Such animals are useful for studying the effects of an Alloiococcus otitidis polypeptide and for identifying and/or evaluating modulators of Alloiococcus otitidis polypeptide infectivity.

A transgenic animal of the invention is created by introducing an Alloiococcus otitidis polypeptide-encoding nucleic acid sequence into the male pronuclei of a fertilized oocyte, e.g., by microinjection, retroviral infection, and allowing the oocyte to develop in a pseudopregnant female foster animal. The human Alloiococcus otitidis cDNA sequence of one or more of the odd numbered sequences set forth in SEQ ID NO:1 through SEQ ID NO: 6649 can be introduced as a transgene into the genome of a non-human animal.

Moreover, a non-Alloiococcus otitidis homologue of the Alloiococcus otitidis gene can be isolated based on hybridization to the Alloiococcus otitidis cDNA (described above) and used as a transgene. Intronic sequences and polyadenylation signals can also be included in the transgene to increase the efficiency of expression of the transgene. A tissue-specific regulatory sequence(s) can be operably linked to the. Alloiococcus otitidis transgene to direct expression of an Alloiococcus otitidis polypeptide to particular cells. Methods for generating transgenic animals via embryo manipulation S and microinjection, particularly animals such as mice, have become conventional in the art and are described, for example, in U.S. Pat. Nos. 4,736,866 and 4,870, 009, U.S. Pat. No. 4,873,191 and in Hogan, 1986. Similar methods are used for production of other transgenic animals. A transgenic founder animal can be identified based upon the presence of the Alloiococcus otitidis transgene in its genome and/or expression of Alloiococcus otitidis mRNA in tissues or cells of the animals. A transgenic founder animal can then be used to breed additional animals carrying the transgene. Moreover, transgenic animals carrying a transgene encoding an Alloiococcus otitidis polypeptide can further be bred to other transgenic animals carrying other transgenes.

In another embodiment, transgenic non-human animals can be produced which contain selected systems that allow for regulated expression of the transgene. One example of such a system is the cre/loxP recombinase system of bacteriophage PL. For a description of the cre/loxP recombinase system, see, e.g., Lakso et al., 1992. Another example of a recombinase system is the FLP recombinase system of Saccharomyces cerevisiae (O'Gon-nan et al., 1991). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al., 1997, and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, e.g., the somatic cell, is isolated.

All patents and publications cited herein are hereby incorporated by reference.

### K. EXAMPLES

The following examples are carried out using standard techniques, which are well known and routine to those of skill in the art, except where otherwise described in detail. The following examples are presented for illustrative purposes only, and should not be construed in any way limiting the scope of this invention.

### Example 1

### Confirmation of the Identity of the Alloiococcus Otitidis 1104-92 Isolate

An Alloiococcus otitidis isolate designated 1104-92 was obtained from Dr. Richard Facklam of the Centers for Disease Control and Prevention in Atlanta. It was isolated from the middle ear fluid of a child in the Atlanta, Ga. area. It was confirmed to be Alloiococcus otitidis by comparing it to the type strain, ATCC51267, obtained from the American Type Culture Collection (Aguirre, 1992 #1). Both the 1104-92 isolate and the type strain were characterized as Gram positive cocci. Both grow on Columbia agar supplemented with 5% yeast extract, 0.5% polysorbate 80 (Tween 80), and 0.7% phospatidyl choline when incubated at 37° C. On this medium, both strains form slow growing small white colonies that require nearly two days to be easily observed with the naked eye. Both are sensitive to lysis by hen egg white lysozyme and Streptococcus globisporus mutanolysin. Both grow in the presence of 2% sodium azide. Both are killed by incubation at 55° C. for 30 minutes. The 1104-92 isolate was subjected to polymerase chain reaction (PCR) identification based on its 16s rRNA gene. This was done using two of the primers specified by Aguirre and Collins (Aguirre, 1992 #2). The antisense primer used was ATCTTCCTGCTTGCAGGAAGAGG (Seq. ID No.: 6653) and the sense primer was ACGCTTCATCTCTGAAGCTAGC (Seq. ID No.6652). PCR confirmed that the 1104-92 isolate was a strain of Alloiococcus otitidis. Thus by multiple criteria, the 1104-92 strain was confirmed to be an isolate of Alloiococcus otitidis.

### Example 2

### Storage, Growth, and Harvest of Alloiococcus Otitidis 1104-92 Isolate for the Isolation of Dna

The Alloiococcus otitidis isolate 1104-92 was stored at −70° C. in Todd-Hewlett broth containing 40% glycerol. A small portion of the frozen stock was streaked onto the agar medium described in Example 1 and incubated at 37° C. for two days. The growth from the plate was swabbed into a 17×100 cm tube containing 6 ml of a serum-free broth medium. This broth medium was prepared with 30 g Todd-Hewlett medium, 5 g yeast extract, 10 ml polysorbate 80 (Tween 80), and 1 liter distilled water. This medium was sterilized by autoclaving for 35 minutes. The bacteria were incubated aerobically without shaking in an aerobic incubator at 37° C. for two days. The tube containing the growing bacteria was then shaken to resuspend the bacteria and added to a liter of the same medium in a Fernbach flask. This flask, in turn, was incubated aerobically for three days without shaking. The bacteria were harvested by first swirling the flask to suspend the bacteria and then low speed centrifugation at about 5,000×g for 30 minutes. The pellet of bacteria was washed by resuspending it in 10 to 15 mL of phosphate buffered saline (PBS), and centrifuging the suspension at about 8,000×g for 20 minutes. The pellet of bacteria was retained and stored frozen at −20° C. The yield of wet bacterial pellet was typically about 1 g per liter of broth.

### Example 3

### Preparation of Alloiococcus Otitidis Genomic Dan

To prepare genomic DNA, 0.95 g frozen pellet of bacteria was defrosted and suspended in 10 mL of PBS containing 1 mM MgCl₂. The bacteria were killed by incubating the suspension at 55° C. for 20 minutes. The suspension was allowed to cool before adding 25 μl of a 10 mg/mL stock of hen egg white lysozyme and 50 μL of a 25,000 unit/mL stock of Streptococcus globisporus mutanolysin to the suspension. It was then incubated for one hour at 37° C. Then 50 μl of a 10 mg/mL stock of RNase was added and the suspension incubated an additional hour at 37° C. After these incubations, sodium dodecylsulfate (SDS) was added to a final concentration of 0.3% (0.3 mL of a 10% stock). This was followed by the addition of 0.3 mL of a 1 mg/mL stock of proteinase K The suspension was then incubated for two hours at 37° C. After this time, an equal volume of water saturated phenol/chloroform/isopropyl (25:24:1) was added to the digested suspension and gently mixed. The upper aqueous layer was retained after a low speed centrifugation and 2.5 volumes of ethanol were added and the tube gently inverted to mix. The DNA was then spooled out on a glass rod and allowed to air dry.

The DNA at this stage still contained obvious impurities and needed further purification. The DNA dried on the glass rod was soaked in 70% ethanol to remove excess phenol and air-dried once again. It was then suspended in 2 ml of Tris-EDTA buffer to which 2 μl of RNase cocktail was added and incubated at room temperature for 75 minutes. Then 100 μl of protease, 100 μl SDS and 40 μl of 100 mM CaCl₂ were added and the suspension incubated for 3.5 hours. An equal volume of chloroform was added, gently mixed, and then centrifuged at a low speed. The aqueous layer was collected and re-extracted with the phenol, chloroform, and isopropyl alcohol reagent. In turn, the aqueous layer was extracted with chloroform. At this point, 3 M sodium acetate was added to the aqueous phase collected form the last extraction and then 3.75 ml of ethanol was added and gently mixed. The DNA was spooled out, soaked in 70% ethanol and allowed to air-dry. The DNA was finally suspended in 2 ml of Tris-EDTA buffer. Based on absorption at 260 nm, the final yield of DNA was 482 μg of DNA. The DNA was confirmed to be that of Alloiococcus otitidis by the PCR method described in example 1. This DNA was submitted for sequencing. The sequences of all the ORFs were obtained as described in Examples 4 and 5 below, and are set forth in odd numbered sequence listings, Seq. ID No. 1 through Seq. ID No. 6649. The sequence of the entire Alloiococcus otitidis genome is set forth in Seq. ID No: 6651.

### Example 4

### Cloning and Sequencing of the Alloiococcus Otitidis Genome

This invention provides nucleotide sequences of the genome of Alloiococcus otitidis, which thus comprises a DNA sequence library of Alloiococcus otitidis genomic DNA. The description that follows provides nucleotide sequences of Alloiococcus otitidis, and also describes how the sequences were obtained and how ORFs (Open Reading Frames) and protein-coding sequences were identified.

To construct a library, genomic DNA was hydrodynamically sheared in an HPLC and then separated on a standard 1% agarose gel. A fraction corresponding to 3000-3500 bp in length was excised from the gel and purified by the GeneClean™ procedure (BIO101, Inc.).

The purified DNA fragments were then blunt-ended using T4 DNA polymerase. The blunt-ended DNA was then ligated to unique BstX1-linker adapters. These linkers are complementary to the pGTC vector, while the overhang is not self-complementary. Therefore, the linkers will not concatermerize nor will the cut-vector religate itself easily. The liner-adapted inserts were separated from the unincorporated linkers on a 1% agarose gel and again purified using GeneClean. The linker-adapted inserts were then ligated to BstX1-cut vector to construct “shotgun” subclone libraries.

Only major modifications to the standard protocols are highlighted. Briefly, the library was transformed into DH10B competent cells (Gibco/BRL, DH5a transformation protocol). Transformed cells were detected by plating onto antibiotic plates containing ampicillin. The plates were incubated overnight at 37° C. Transformant clones were then selected for sequencing. The cultures were grown overnight at 37° C. DNA was purified using a silica bead DNA preparation (Egelstein, 1996) method. In this manner, 25 mg of DNA was obtained per clone.

These purified DNA samples were then sequenced using ABI dye-terminator chemistry. All subsequent steps were based on sequencing by automated DNA sequencing methods. The ABI dye terminator sequence reads were run on MegaBace™ 10000 (Amersham) machines and the data transferred to UNIX based computers. Base calls and quality scores were determined using the PHRED software program (Ewing et al., 1998, Genome Res. 8: 175-185; Ewing and Green, 1998, Genome Res. 8:685-734). Reads were assembled using PHRAP (P. Green, Abstracts of DOE Human Genome Program Contractor-Grantee Workshop V, Jan.1996, p 157) with default program parameters and quality scores.

To identify Alloiococcus otitidis genome encoded polypeptides, the complete genomic sequence of Alloiococcus otitidis was analyzed essentially as follows: First, all possible stop-to-stop open reading frames (ORFs) greater than or equal to 222 nucleotides in all three reading frames were translated into amino acid sequences.

Second, the identified ORFs were analyzed for homology to known protein sequences. Third, the coding potential of non-homologous sequences was evaluated with the GeneMark™ software program (Borodovsky and Mcininch, 1993, Comp. Chem. 17:123).

### Example 5

### Bioinformatics and Gene Mining of Alloiococcus Otitidis

The genomic sequence of Alloiococcus otitidis was examined for suitable open reading frames. ORFs were determined in the following manner. An ORF was defined as having one of three potential start site codons, ATG, GTG, or TTG and one of three potential stop codons, TAA, TAG, TGA. The Applicants used a unique set of three ORF finder algorithms: GLIMMER™ (Salzberg et al., 1998), GeneMark™ (Lukashin and Borodovsky, 1998) and Applicants' assignee's own program to enhance the efficiency for finding “all” ORFs. In order to evaluate the accuracy of the ORFs determined, a program developed by Applicants' assignee called DiCTion was employed that uses a discrete mathematical cosine function to assign a score for each ORF. An ORF with a DiCTion score >1.5 was considered to have a high probability of encoding a protein product. The minimum length of an ORF predicted by the three ORF finders was set to 225 nucleotides (including stop codon) which would encode a protein of 74 amino acids. An in-house graphical analysis program developed by Applicants' assignee also allowed the Applicants to see all six reading frames and the location of the predicted ORFs relative to the genomic sequence. This helped to eliminate those ORFs that have large overlaps with other ORFs, although there are known cases of ORFs being totally embedded within other ORFs (Loessner et al., 1999; Hernandez-Sanchez et al., 1998).

The initial annotation of the Alloiococcus otitidis ORFs was performed using the BLAST (v. 2.0) Gapped search algorithm, BlastP, to identify homologous sequences (Altschul et al., 1997). A cutoff ‘e’ value of anything <e⁻¹⁰ was considered significant. Other search algorithms such as FASTA or PSI-BLAST were used as needed. The non-redundant protein sequence database used for the homology searches consisted of GenBank, SWISS-PROT, PIR, and TREMBL database sequences. ORFs with a BlastP result of >e⁻¹⁰ were considered to be unique to Alloiococcus otitidis.

A keyword search of the entire BLAST results was carried out using known or suspected target genes (those that encode polypeptide candidates for inclusion in immunogenic compositions) as well as words that identified the location of a protein or function.

Several parameters are used to determine partitioning of the predicted proteins. Proteins destined for translocation across the cytoplasmic membrane encode a leader signal (also called signal sequence) composed of a central hydrophobic region flanked at the N-terminus by positively charged residues (Pugsley, 1993). A program called SignalP identifies signal peptides and their cleavage sites (Nielsen et al., 1997). To predict protein localization in bacteria, the software PSORT has been used (Nakai and Kanehisa, 1991). This program uses a neural net algorithm to predict localization of proteins to the ‘cytoplasm', ‘periplasm’, and ‘cytoplasmic membrane’ for Gram-positive bacteria as well as ‘outer membrane’ for Gram-negative bacteria. Transmembrane (TM) domains of proteins have been detected using the software program TopPred2 (Cserzo et al., 1997).

The Hidden Markov Model (HMM) Pfam database of multiple alignments of protein domains or conserved protein regions (Sonnhammer et al., 1997) was used to identify Alloiococcus otitidis proteins that may belong to an existing protein family. Keyword searching of this output was used to help identify additional candidate polypeptides that may have been missed by the BLAST search criteria. Additional HMM models were developed by Applicants' assignee. A computer algorithm, called HMM Lipo, was developed internally to predict lipoproteins using 131 biologically proven bacterial lipoproteins. This training set was generated from experimentally proven prokaryotic lipoproteins. The protein sequence from the start of the protein to the cysteine amino acid plus the next two additional amino acids was used to generate the HMM. Using approximately 70 known prokaryotic proteins containing the LPXTG cell wall sorting signal, a HMM (Eddy, 1996), was developed to predict cell wall proteins that are anchored to the peptidoglycan layer (Mazmanian et al., 1999; Navarre and Schneewind, 1999). The model used not only the LPXTG sequence, but also included two features of the downstream sequence, the hydrophobic transmembrane domain and the positively charged carboxy terminus. There are also a number of proteins that interact, non-covalently, with the peptidoglycan layer and are distinct from the LPXTG protein class described above. These proteins seem to have a consensus sequence at their carboxy terminus (Koebnik, 1995). The Applicants also have developed and used a HMM of this region to identify any Alloiococcus otitidis that may fall into this class of proteins.

The proteins encoded by Alloiococcus otitidis identified ORFs were also evaluated for other useful characteristics. A tandem repeat finder (Benson, 1999) identified ORFs containing repeated DNA sequences such as those found in MSCRAMMs (Foster and Hook, 1998) and phase variable surface proteins of Neisseria meningitidis (Parkhill et al., 2000). Proteins that contain the Arg-Gly-Asp (RGD) attachment motif, together with integrins that serve as their receptor, constitute a major recognition system for cell adhesion. RGD recognition is one mechanism used by microbes to gain entry into eukaryotic tissues (Stockbauer et al., 1999; lsberg and Tran Van Nhieu, 1994). However, not all RGD containing proteins mediate cell attachment. It has been shown that RGD-containing peptides with a proline at the carboxy end (RGDP) are inactive in cell attachment assays (Pierschbacher and Ruoslahti, 1987) and were excluded from consideration. The Geanfammer software was used to cluster proteins into homologous families (Park and Teichmann, 1998). Preliminary analysis of the family classes has provided novel ORFs within a polypeptide candidate cluster, as well as defining potential protein function.

### Example 6

### Proteomics

Two strategies for identifying surface exposed proteins on microorganisms are used to identify surface exposed proteins on Alloiococcus otitidis. The first involves digesting of the surface of live organisms with protease and analyzing the released proteins. The second involves the purification of cellular membrane in a way that preserves the interaction of proteins that normally associate with the membrane. Both techniques revolve around the use of mass spectroscopy to identify the proteins that are detected but otherwise differ substantially.

In the first strategy, cells are treated with proteolytic enzymes in isotonic solution without substantial cell lysis. Experimentally, the cells are washed and suspended in an isotonic glycerol solution of neutral pH. A reductant, dithiothreitol, is first added to cleave the protein disulfide linkages and then iodoacetamide is added to alkylate the exposed cysteine thiols. The objective of this step is to break up the secondary and tertiary structure of the surface proteins. Porcine trypsin is then added and the cells are incubated at 37° C. for 4 to 24 hours. The cells are then spun down and the supernatant containing the digest of surface exposed proteins is analyzed by mass spectroscopy (vide infra). At the end of the procedure, cell integrity is observed by Scanning Electron Microscopy (SEM). Cells, so treated, have a strikingly different morphology, as seen under the SEM, which is best described as a “clean shave”. That is, the normal spongy or furry appearance is replaced by a smooth spherical morphology. Maintaining cell integrity throughout this operation is important. The intact cell membrane protects all intracellular proteins from the proteolytic step required for identification. Any ruptured cells therefore spill intracellular proteins into the extracellular environment where they are digested and erroneously identified as surface exposed proteins.

In the second strategy, proteins with a natural tropism for membranes are copurified with membranes by simple centrifugation. In principle, this separates proteins into two classes; soluble and membrane bound. In this case, the class of interest is membrane bound because many of these are also surface exposed as well. Experimentally, cells are ruptured in the absence of detergent by sonication or shear and the lysate is subjected to differential centrifugation. The pellet from a high speed spin (20,000×g) consists of membranes and their associated proteins. This pellet is then dissolved in SDS load buffer and then separated by SDS PAGE. The gel, after staining, shows a large number of still intact proteins that share the quality of being membrane bound. The protein bands from this gel are then cut and exposed to trypsin for the obligate digestion before mass spectroscopic identification.

It should be pointed out that many of these proteins are actually on the inside (cytoplasmic) surface of the membrane and may well have no surface exposure at all. There are several advantages to this method, however, that offset this apparent lack of selectivity. Many proteins are not apprehended by the aforementioned technique of surface digestion are, however, captured by membrane enrichment. Surface digestion fails for proteins that are minimally exposed to the protease. This would be the case for proteins that are highly structured, membrane buried, or heavily glycosylated. In the final analysis both techniques have different merits and shortcomings and can be used in a complementary way.

In a typical example, a membrane preparation was dissolved in SDS load buffer and the proteins separated by SDS PAGE. After staining with colloidal Coomassie Blue stain, fifteen gel bands containing the entire sample lane were excised and the bands digested individually with trypsin. The tryptic peptides were analyzed using microcapillary reversed-phase liquid chromatography—nano-electrospray tandem mass spectrometry (LC-MS/MS) on a Finnigan LCQ Deca quadrupole ion trap mass spectrometer. Acquisition was performed using a “3 CADs experiment”. Using SEQUEST software, the uninterpreted tandem mass spectrometric information was used to search appropriate sequence databases to identify the proteins present in each gel band.

### Example 7

### Protein Identification

In order to identify the polypeptide components in the complex surface digest mixture, an analytical technique is used to separate and sequence multiple peptides with high sensitivity over a large concentration range. Tandem mass spectrometry (MS/MS) has been shown to be a powerful approach to analyze proteins from both gels and in solution. MS/MS first uses a mass analyzer to separate a peptide ion from a mixture of ions, then uses a second step or mass analyzer to activate and dissociate the ion of interest. This process, known as collision induced dissociation (CID) causes the peptide to fragment at the peptide bonds between the amino acids, and therefore, the fragmentation pattern of a peptide can be used to determine its amino acid sequence. In addition, the SEQUEST computer algorithm can be used to search the experimental fragmentation spectrum directly against protein or translated nucleic acid sequence databases. For peptides above roughly 800-900 Da in size, a single spectrum can uniquely identify a protein.

To sequence multiple peptides from a complex mixture, a reversed phase chromatography system is coupled to an electrospray ion trap mass spectrometer. In this system, it is known that high sensitivity (down to sub-femtomole levels) is attained by minimizing both flow rate and column diameter to concentrate the elution volume and direct as much of the column effluent as possible into the orifice of the mass spectrometer detector. Initial experiments separated peptides using a reversed phase gradient of 1% acetonitrile/minute. In order to increase chromatographic separation, longer gradients, down to 0.28% acetonitrile/minute, and slower flow rates (50 nL/min) will be employed. To maximize the coverage of proteins present in the sample, the data-dependent acquisition feature of the ion trap is employed. The instrument is programmed to acquire a single ms scan in the range of m/z 375-600, then to obtain ms/ms data on the top two ions in that range. It then acquires another ms scan in the range of m/z 600-1000, and obtains ms/ms data on the top two ions in that range. This results in better coverage of the larger peptides.

Dynamic exclusion is used to prevent reacquisition of tandem mass spectra of ions once a spectrum had been acquired for a particular m/z value. The isotopic exclusion function excludes the ion associated with the ¹³C isotope of peptides from the list of ions slated for MS/MS. A 3-μ mass width window is selected for this purpose. Using these data-dependent features dramatically increases the number of peptide ions that will be selected for CID analysis.

### Example 8

### Sequest Analysis

The LC-MS/MS data acquisition conditions described above typically results in fragmentation data for more than 2000 peptide ions for each run. Using the Sequest algorithm, this data is searched against a composite protein sequence database containing the translated ORFs from Alloiococcus otitidis combined with the non-redundant protein sequence database OWL. Sequest search conditions using a modified trypsin selectivity allows a differential search of +16 Da on methionine (to account for met oxidation). Candidate matches identified by Sequest are confirmed using the following manual procedure. Those matches with Xcorr values greater than 2.5 (a measure of the similarity of the experimental ms/ms data to that generated from the sequence database), and delCn values greater than 0.1 (delCn measures the normalized difference between the Xcorr values of the first and second matches), are chosen for further analysis. The fragmentation spectra from good matches are checked for reasonable signal/noise, and the list of matched ions are examined for reasonable continuity. Some matches that are not acceptable alone are only included if other confirmatory ms/ms data is generated by the same sample.

### Example 9

### Cloning and Protein Expression

Primer sets are designed for PCR amplification of desired ORFs such that the forward 5′ primer anneals at the start of the predicted mature protein. For lipoproteins, the 5′ forward primer is designed to anneal just after the codon encoding a cysteine residue of the mature protein to minimize disulfide bridging. Design of the opposing reverse 3′ primers independent upon the type of predicted protein. For those proteins that contain an LPXTG, the primer is designed such that it anneals at the beginning (5′ end) of the cell wall anchor region. For all other predicted proteins, primers are redesigned such that they anneal at the 3′ end of the ORF. Additionally, the 5′-forward primer is initially designed to allow an in-frame fusion to thioredoxin with the opposing 3′-reverse primer allowing read-through to include a downstream his-patch and V5 epitope (pBAD/thio-TOPO®, Invitrogen, Carlsbad, Calif.). In parallel, these same PCR products are cloned into pCR®T7TOPO® (Invitrogen, Carlsbad, Calif.). This allows for an N-terminal fusion to a Xpress epitope and a his-tag for purification.

All PCR reactions are performed using Alloiococcus otitidis chromosomal DNA as the template. PCR products are transformed into the E. coli host, TOP10, and plated on SOB containing 100 μg/mL ampicillin. Colonies are screened by PCR amplification using a vector specific 5′ primer and the specific 3′ reverse primer annealing to the gene insert. Colonies are seeded into wells of 96 well microtiter plates containing 50 μL 50% glycerol. Ten-12 colonies per gene are seeded in one row of the plate. In a second 96 well PCR plate, 50 μL reactions are set up specific to the gene of interest. One μL of the cells suspended in glycerol is used as template in the PCR reaction. Reactions that produce bands of the expected size are analyzed further. The cells that are seeded in 50% glycerol have SOB media added to them and incubated at 37° C. for 5-8 hours and frozen at −70° C.

PCR positive colonies are inoculated into 2 mL cultures for overnight growth. Part of the culture is used to prepare plasmid DNA that is then analyzed by restriction digestion to confirm the inserts while another part is used to seed 10 mL expression cultures (for pBAD plasmids) for expression. Mid-log phase cultures are induced with 0.5% L-arabinose for 2-3 hours. T7/NT plasmids are transformed into the expression strain BLR(DE3) pLysS before screening. T7/NT cultures are induced by the addition of 1 mM IPTG and incubated for 2 hours. Whole cell lysates of induced cultures are run on SDS-PAGE in duplicate. One gel is stained with Coomassie and the other transferred to nitrocellulose and probed with antibody to the relevant epitope tag.

Positive clones are grown in 1-2 L volumes and induced for large-scale purification. Solubility and expression level of the recombinant proteins are assessed by freeze-thaw lysis of the cells, followed by DNase/RNase digestion and centrifugation at 9,000×g for 15 minutes in a RC5B refrigerated centrifuge. The soluble fraction is removed from the insoluble material and both are then separated and evaluated for protein localization and expression by SDS-PAGE. Soluble fusion proteins are purified by passing the soluble fraction of lysed cells over Ni-NTA (Qiagen) resin and eluting the bound proteins with imidazole. Eluted proteins are buffer exchanged on PD-10 columns (Amersham Pharmacia Biotech, Piscataway, N.J.).

Insoluble recombinant proteins are washed using centrifugation 3 times in PBS, 0.1% Triton-X100. The inclusion bodies are then solubilized in PBS-4 M urea and buffer exchanged through a PD-10 column (Amersham Pharmacia) into PBS, 0.01% Triton-X100, 0.5 M NaCl. Proteins are quantitated by the Lowry assay (Lowry et al., 1951), and checked for purity and concentration by SDS-PAGE.

### Example 10

### Generation of Polyclonal Antisera and Monoclonal Antibodies

To generate monoclonal antibodies, female, eight week old Blab/c mice are inoculated intraperitoneally with the gene products three times in a four week period (week 0, 2, 4). The dosage of the protein gene product is between 5 and 50 go. Two different fusion procedures are performed. The first fusion is done after a three-month rest period and an intraperitoneal boost with the protein; the second fusion is done after a four-month rest period and a boost by the same route. During the immunization and rest period, mouse sera recollected and tested for antibody activity by ELISA using the protein as the coating antigen.

For both fusions, the spleens are excised from two immunized mice about 72 hours after the last injection and mixed with the nonsecreting X63Ag8.653 mouse (Balb/c) myeloma cells in a 5:1 ratio (splenocytes:myeloma). The cells are allowed to fuse for four minutes in 50% (wt/wt) polyethylene glycol 1500 and 10% dimethyl sulfoxide in Dulbecco's Modified Eagle medium (D-MEM). Multiple dilutions of the fused cells are then made into the selection medium, i.e., D-MEM supplemented with hypoxanthine, aminopterin, thymine, 10% fetal bovine serum, and 10% NCTC-109 medium supplement (Gibco-BRL). The reactivity in each dilution is evaluated by SDS-PAGE western blots, dot blots, and ELISA using the gene product protein and whole bacterial cells as. the, detection antigens. The positive reacting hybridomas are identified and saved. These hybridomas are then further subcloned by the limiting dilution procedure to isolate the individual antibody secreting cells. The selected hybridoma cells are then used to prepare the monoclonal antibodies. This is either performed by growing them in D-MEM and concentrating the supernatant by ammonium sulfate precipitation, or by injecting mice with the hybridoma cells and collecting the resulting ascites fluid.

### Example 11

### Elisa Titration of Mouse and Human Sera, Monoclonal Antibodies, and Mucosal Washings Against Gene Products and Whole Bacteria

The levels of antibodies elicited by the gene products cloned and expressed as per Example 10 toward themselves and whole bacterial cells are measured by Enzyme linked immunosorbent assay (ELISA). For the whole cell ELISA, the bacteria are grown overnight on agar medium and swabbed into PBS. The turbidity of the cells is adjusted to 0.10 at 600 nm and 100 μl added to the wells of a 96 ELISA plate. The cells are dried overnight at 37° C., sealed with a mylar plate sealer and stored at 4° C. until needed. On the day of the assay, the residual protein binding sites are blocked by adding 5% non-fat dry milk in PBS with 0.1% Tween 20 (BLOTTO) and incubating at 37° C. for one hour. The blocking solution is then removed and 100 μl of sera serially diluted in the wells with BLOTTO. The sera are allowed to incubate for 1 h at 37° C. The plate wells are soaked with 300 ml PBS containing 0.1% Tween 20 for 30 seconds and washed 3 times for 5 seconds with a SkanWasher 300 plate washer (Skatron Instruments AS, Norway) and then incubated 1 hr at 37° C. with goat anti-mouse IgG conjugated to alkaline phosphatase (BioSource International) diluted 1:1000 in BLOTTO. After washing, the plates are developed at room temperature with 100 μl per well of 1 mg/ml p-nitrophenyl phosphate dissolved in diethanolamine buffer. Development is stopped by adding 50 μl of 3N NaOH to each well. The absorbance of each well is read at 405 nm and titers calculated by linear regression. The titer is reported as the inverse of the dilution extrapolated to an absorption value of 0.10 units.

For the antigen specific ELISA's, the proteins are diluted to a concentration of 5 μg/ml in a 50 mM sodium carbonate buffer (pH 9.8) containing 0.02% sodium azide (Sigma Chemical Co.). One hundred micro liters is added to each well of a 96 well E.I.A./R.I.A medium binding ELISA plate (Costar Corp., Cambridge, Mass.) and incubated for 16 hours at 4° C. The plates are washed and subsequently treated the same as described for whole cell ELISA procedure except, in addition to dilutions of sera, mucosal washings and monoclonal antibody suspensions are tested as well.

### Example 12

### Immunogold Labeling of Alloiococcus Otitidis and low voltage Scanning Electron Microscopy

Surface exposure of proteins on Alloiococcus otitidis is assessed by immunogold labeling of whole bacteria and electron microscopy. Bacteria cells are labeled as previously described (Olmsted et al., 1993). Briefly, late-log phase bacterial cultures are washed twice, and resuspended to a concentration of 1×10⁸ cells/ml in 10 mM phosphate buffered saline (PBS) (pH 7.4) and placed on poly-L-lysine coated glass coverslips. Excess bacteria are gently washed from the coverslips and unlabeled samples are placed into fixative (2.0% glutaraldehyde, in a 0.1 M sodium cacodylate buffer containing 7.5% sucrose) for 30 min. Bacteria to be labeled with colloidal gold are washed with PBS containing 0.5% bovine serum albumin, and the pre-immune or hyper-immune mouse polyclonal antibody prepared above applied for 1 hour at room temperature. Bacteria are then gently washed, and a 1:6 dilution of goat anti-mouse conjugated to 18 nm colloidal gold particles (Jackson ImmunoResearch Laboratories, Inc., West Grove, Pa.) applied for 10 min at room temperature. Finally, all samples are washed gently with PBS, and placed into the fixative described above. The fixative is washed from samples twice for 10 min in 0.1 M sodium cacodylate buffer, and posffixed for 30 min in 0.1 M sodium cacodylate containing 1% osmium tetroxide. The samples are then washed twice with 0.1 M sodium cacodylate, dehydrated with successive concentrations of ethanol, critical point dried by the CO₂ method of Anderson (Anderson, 1951) using a Samdri-780A (Tousimis, Rockville, Md.), and coated with a 1-2 nm discontinuous layer of platinum. Alloiococcus otitidis cells are then viewed with a LEO 1550 field emission scanning electron microscope operated at low accelerating voltages (1-4.5 keV) using a secondary electron detector for conventional topographical imaging and a high-resolution Robinson backscatter detector to enhance the visualization of colloidal gold by atomic number contrast.

### Example 13

### Intranasal and Parenteral Immunization of Mice

Six-week old, pathogen-free mice are purchased from Jackson Laboratories (Bar Harbor, Maine) and housed in cages under standard temperature, humidity, and lighting conditions. The mice, at 10 animals per group, are immunized with an appropriate amount of the protein(s) to be tested. For parenteral immunization, the protein is mixed with 100 μg of monophosphoryl lipid A (MPL™) (Corixa, Hamilton, Mont.) per dose to a final volume of 200 μl in saline and then injected subcutaneously (SC) into mice. All groups receive a booster with the same dose and by the same route 3 and 5 weeks after the primary immunization. Control mice are injected with MPL™ alone. All mice are bled two weeks after the last boosting; sera is then isolated and stored at −20° C. For intranasal (IN) immunization, mice receive three IN immunizations one week apart. On each occasion, an appropriate dose of the protein to be tested is formulated with 0.1 μg of CT-E29H, a genetically modified cholera toxin that is reduced in enzymatic activity and toxicity (Tebbey et al., 2000), and slowly instilled into the nostril of each mouse in a 10 μl volume. Mice immunized with CT-E29H alone are used as controls. Serum samples are collected one week after the last immunization.

### Example 14

### In vitro Opsonphagocytosis Analysis

An in vitro opsonic reaction, that may mimic the in vivo reaction, is achieved when a mixture of Alloiococcus otitidis cells, heat inactivated human serum containing specific antibodies to the alloiococcal strain, and an exogenous complement source are incubated together. Opsonophagocytosis proceeds during incubation of freshly isolated human polymorphonuclear cells (PMN's) and the antibody/complement/alloiococcal cell mixture. Bacterial cells that are coated with antibody and complement are killed upon opsonophagocytosis. Colony forming units (cfu) of surviving bacteria that escape from opsonophagocytosis are determined by plating the assay mixture. Titers are reported as the reciprocal of the highest dilution that gives ≧50% bacterial killing, as determined by comparison to assay controls. Specimens, which demonstrate less than 50% killing at the lowest serum dilution tested (1:8) are reported as having an OPA titer of 4. The highest dilution tested is 1:2560. Samples with ≧50% killing at the highest dilution are repeated, beginning with a higher initial dilution.

The method described herein is a modification of Gray's method (Gray, B. M. 1990). The assay mixture is assembled in a 96-well microtiter tissue culture plate at room temperature. The assay mixture consists of 10 μL of test serum (a series of two-fold dilutions) heated to 56° C. for 30 minutes prior to testing, 10 μL of preclostral bovine serum (complement source) having no opsonic activity for the bacterial test strain, and 20 μL of buffer containing viable Alloiococcus otitidis organisms. This mixture is incubated at 37° C. without CO₂ for 30 minutes with shaking. Next, 40 μL of human PMNs, freshly prepared from heparinized peripheral blood by dextran sedimentation and Percoll density centrifugation, suspended in buffer at a concentration of 1×10⁶/mL is added. The assay plate(s) is then incubated at 37° C. for an additional 90 minutes with vigorous shaking. Aliquots from each well are dispensed onto the upper ¼ of a 15×100 mm blood agar plate. The blood agar plate is tilted while pipetting to allow the liquid suspension to “run” down the plate. Plates are incubated overnight in 5% CO₂ at 37° C. The viable cfu are counted the following morning. Negative control wells, lacking bacterial cells, test serum, complement and/or phagocytes in appropriate combination are included in each assay. A test serum control, which contains test serum plus bacterial cells and heat-inactivated complement, is included for each individual serum. This control is used to assess whether the presence of antibiotics or other serum components are capable of killing the bacterial strain directly (Le. in the absence of complement or PMN's). A human serum with known opsonic titer is used as a positive human serum control. The opsonic antibody titer for each unknown serum is calculated as the reciprocal of the initial dilution of serum giving 50% cfu reduction compared to the control without serum.

### Example 15

### Mouse Nasal Clearance Model

The gene products are evaluated in a mouse nasal clearance model. Groups of at least ten mice, of either the C3H/heJ or Balb/C strains, are immunized as described above. Approximately two weeks after the last immunization, the mice are anesthetized with 1.2 mg of ketamine HCl (Ft. Dodge Laboratories, Ft. Dodge, Iowa) by i.p. injection. The mice are then challenged intranasally by placing 10 μL of a suspension the 1104-92 isolate of Alloiococcus otitidis prepared to an A₅₅₀ of 0.5 in the nose and allowing the animal to inhale it. The 1104-92 isolate used for this challenge is passaged twice in mice by intranasal instillation to select for a variant with optimal colonization capacity. After five hours the mice are killed, their nasal turbinates removed, homogenized in 2 ml of sterile saline, appropriately diluted, and plated onto selective agar medium. The selective medium is a Columbia based agar supplemented with 0.5% Tween-80, 0.5% azide and 20 mg/L erythromycin. Owing to its slow growth, Alloiococcus otitidis requires 3 to 4 days before colonies a visible on this medium. The degree of clearance of the immunized mice is calculated as the percent decrease in colony forming units (CFUs) and statistically compared to a set of mice immunized with an unrelated protein such as CRM, Keyhole Limpet Hemocyanin (KLH) or other protein not derived from Alloiococcus otitidis.

### L. REFERENCES

U.S. Pat. No. 4,196,265
U.S. Pat. No. 4,522,811
U.S. Pat. No. 4,554,101
U.S. Pat. No. 4,683,195
U.S. Pat. No. 4,683,202
U.S. Pat. No. 4,736,866
U.S. Pat. No. 4,816,567
U.S. Pat. No. 4,837,151
U.S. Pat. No. 4,870,009
U.S. Pat. No. 4,873,191
U.S. Pat. No. 4,873,316
U.S. Pat. No. 4,987,071
U.S. Pat. No. 5,116,742
U.S. Pat. No. 5,168,062
U.S. Pat. No. 5,223,409
U.S. Pat. No. 5,328,470
U.S. Pat. No. 5,580,859
U.S. Pat. No. 5,643,576
U.S. Pat. No. 6,127,170
U.S. Pat. No. 6,113,918
U.S. Pat. No. 6,168,918
U.S. Pat. No. 6,168,943
U.S. Pat. No. 6,207,646
International Application No. EP 125,023
International Application No. EP 171,496
International Application No. EP 184,187
International Application No. EP 264,166
International Application No. EP 524,800
International Application No. EP PCT/US86/02269
International Application No. WO 00/18434
International Application NO. WO 00/36776
International Application No. WO 86/01533
International Application No. WO 90/02809
International Application No. WO 91/17271
International Application No. WO 92/01047
International Application No. WO 92/09690
International Application No. WO 92/15679
International Application No. WO 92/18619
International Application No. WO 92/19265
International Application No. WO 92/20791
International Application No. WO 93/01288
International Application No. WO 93/13302
International Application No. WO 97/07668
International Application No. WO 97/07669
International Application No. WO 00/63364
Abravaya et al., Nucleic Acids Res., 23:675-682, 1995
Adams et al., Nature 355:632-634, 1992
Adams et al., Nature 377 Supp:3-174, 1995
Adams et al., Science 252:1651-1656,1991
Aguiree, M., and M. D. Collins, 1992 Phylogenetic analysis of Alloiococcus otitis I gen. Nov., sp. Nov., and organism from human ear fluid: Internat. J. System Bacteriol., v.42, p. 79-83.
Altschul, et al, “Gapped BLAST and PSI—BLAST” a generation of protein database search programs” Nuc. Acids Res. 25(17):33890-402, 1997
Altschul et al, J. Molec. Biol. 215:403-410, 1990
Amann et al., Gene 69:301-315,1988.
Anderson, “Techniques for the preservation of three-dimensional structure in preparing specimens for the electron microscope.” Trans. N.Y. Acad. Sci. 13(130): 130-134, 1951
Arlinghaus et al., Analysis of biosensor chips for identification of nucleic acids, Analytical Chemistry 69(18):3747-53, 1997.
Bairoch and Apweiler, Nucleic Acids Research, 28:45-48, 2000.
Baldari et al, A novel leader peptide which allows efficient secretion of a fragment of human interleukin 1 beta in Saccharomyces cerevisiae, EMBO Journal., 6(1):229-34, 1987.
Baneji et al., Cell,33:729-740; 1983
Barker et al., Nucleic Acids Research, 29:29-32, 2001.
Bartel and Szostak, Science 261:1411-1418, 1993.
Bartel et al., Biotechniques 14:920-924, 1993(b).
Bartel, “Cellular Interactions and Development: A Practical Approach” I, pp. 153-179, 1993(a).
Bateman et al., The Pfam protein families database,” Nucleic Acid Res., 28(1), 263-266, 2000.
Benson, “Tandem repeats finder: a program to analyze DNA sequences,: Nucleic Acids Res. 27(2):573-80, 1999.
Beswick, A. J., B. Lawley, and A. P. Fraise, 1999, Detection of Alloiococcus otitis in mixed bacterial populations from middle-ear effusions of patients with otitis media: Lancet v.354, p.386-389.
Boliver et al., Construction and characterization of new cloning vehicles. II. A multipurpose cloning system, Gene, 2(2):95-113, 1977.
Borodovsky and Mclinich 1993, Comp. Chem. 17:123.
Bradley, Current Opinion in Biotechnology 2:823-829, 1991
Bradley, in “Teratocarcinomas and Embryonic Stem Cells: A Practical Approach,” E. J. Robertson, ed., IRL, Oxford, pp.113-152, 1987.
Briles et al., “Intranasal immunization of mice with a mixture of the pneumococcal proteins PsaA and PspA is highly protective against nasopharyngeal carriage of Streptococcus pneumoniae,” Infect Immun. 68(2):796-800, 2000.
Bunzow et al., Nature, 336:783-787, 1988.
Burge and Karlin, “Prediction of complete gene structures in human genomic DNA.”J. Mol. Biol. 268:78-94, 1997.
Butler et al., “Pneumococcal vaccines: history, current status, and future directions,: Am. J. Med. 107(1A):69S, 76S, 1999.
Byrne and Ruddle, PNAS 86:5473-5477, 1989.
Calame and Eaton, Adv. Immunol. 43:235-275, 1988.
Campes and Tilghman, Genes Dev. 3:537-546, 1989.
Chang, et al., Phenotypic expression in E. coil of a DNA sequence coding for mouse dihydrofolate reductase, Nature 275(5681), Oct. 19, 1978.
Chen et al., PNAS 91:3054-3057, 1994.
Cohen et al., Adv. Chromatogr. 36:127-162, 1996.
Cotton et al., PNAS 85:4397, 1988.
Cotton, Mutat Res. 285-125-144, 1993.
Cowan et al., “RGS Proteins: Lessons from the RGS9 subfamily,” Progress in Nucleic Acid Research and Molecular Biology 65:341-359, 2001.
Crain et al., “Streptococcus pneumoniae surface protein A (PspA) is serologically highly variable and is expressed by all clinically important capsular serotypes of Streptococcus pneumoniae.: Infect. Immun. 58(10):3293-9, 1990.
Cserzo et al., “Prediction of Transmembrane alpha-helices in prokaryotic membrane proteins: the dense alignment surface method,” Protein Engineering 10(6):673-6, 1997.
DeRisi et al., Use of cDNA microarray to analyze gene expression patterns in human cancer, Nature Genetics Dec 14(4):457-460, 1996.
D'Eustachio et al., Science 220: 919-924, 1983.
Devereux et al., “Competence and virulence of streptococcus pneumoniae: Adc and PsaA mutants exhibit a requirement for Zn and Mn resulting from inactivation of putative ABC metal permeases,” Mol. Microbiol. 25(4):727-739, 1997.
Dintilhac, et al., “Competence and virulence of Streptococcus pneumoniae: Adc and PsaA mutant exhibit a requirement for Zn and Mn resulting from inactivation of putative ABC metal permeases,” Mol. Microbiol. 25(4):727-739, 1997.
Doestschman et al., “Antibody response to pneumococcal vaccination in children younger than five years of age,” J. Infect.Dis. 148:131-137, 1983.
Douglas et al, “Antibody response to pneumococcal vaccination in children younger than five years of age,” J. Infect. Dis. 148:131-137, 1983.
Eddy, “Hidden Markov Models: Current Opinion in Structural Biology 6(3):361-5, 1996.
Edlund et al., Science230:912-916, 1985.
Eichelbaum, Clin. Exp. Pharmnacol Phsiol, 23(10-11):983-985, 1996.
Elledge et al., Proc. Natl. Acad. Sci. USA, 88:1731-1735, 1991.
Eng, McCormack and Yates, “An approach to correlate tandem mass-spectral data of peptides with amino-acid sequences in a protein database,” Journal of the American Society for Mass Spectrometty,” 5:976-989, 1994.
Ewing et al., 1988., Genome Res. 8:175-185.
Ewing and Green, 1988, Genome Res. 8:685-734. Reads were assembled using PHRAP (P. Green, Abstracts of DOE Human Genome Program Contractor-Grantee Workshop V, Jan.1996, p 157.)
Faden, H., and D. Dryja, 1989, Recovery of a unique bacterial organism in human middle ear fluid and its possible role in chronic otitis media: J. Clin. Microbiol., v. 27, p.2488-2491.
Fan, Y. et al., PNAS, 87:6223-27, 1990
Finely et al., Proc. Natl. Acad. Sci. USA, 91:12980-12984, 1994.
Foster and Hook, “Surface protein adhesins of Staphylococcus aureus,: Trends Microbiol. 6(12):484-8, 1998.
Fraser et al., Genomic sequence of a Lyme disease spirochaete, Borrelia burgdorferi” Nature 390(6660):580-6, 1997.
Frohman et al, Proc. Natl. Acad. Sc. USA 85, 8998-9002, 1988.
Gabizon, et al., Effect of liposome composition and other factors on the targeting of liposomes to experimental tumors: biodistribution and imaging studies, Cancer Research 50(19):6371-6378, Oct. 1, 1990.
Gaultier et al, Nucleic Acids Res.156625-6641, 1987.
Gentz et al., Proc. Natl. Acad. Sci. USA, 86:821-824, 1989.
Goldstein and Garau, ”30 years of penicillin-resistant S pneumoniae: myth or reality?,” Lancet 350(9073):233-4.
Gray, Conjugate Vaccines Supplement p694-697, 1990.
Griffen et al., Appl. Biochem Biotechnol. 38:147-159, 1993.
Gunnar von Heijne, “Membrane Protein Structure Prediction, Hydrophobicity Analysis and the Positive-inside Rule” J. Mol. Biol., 225:487-494, 1992.
Harlow and Lane, “Antibodies: A Laboratory Manual,” Cold Spring Harbor Laboratory Press, Cold Spring Harbor, N.Y., 1988.
Harper et al., Cell, 75:805-816, 1993.
Haselhoff and Gerlach, Nature 334:585-591, 1988.
Haselhoff and Gerlach, 1988 (p. 634).
Hausdorff et al., “Which pneumococcal serogroups cause the most invasive disease: implications for conjugate vaccine formulation and use, part I,: Clinical Infectious Diseases 30(1):100-21, 1997.
Hayashi, Genet Anal. Tech. Appl. 9:73-79, 1992.
Helene et al., Ann N.Y. Acad. Sci. 660:27-36, 1992.
Helene et al., Anticancer Drug Des. 6(6):569-84, 1991.
Hendolin, P. H., U Karkkainen, T. Himi, A. Markanen, and J. Ylikoski, 1999, High incidence of Alloiococcus otitis in otitis media with effusion: Pediatr Infect. Disc. J., v.18, p. 860-865.
Hendolin, P. H., A. Markkanen, J. Ylikoski, and J. J. Wahifors, 1997, Use of multiplex PCR for simultaneous detection of four bacterial species in middle ear effusions: J. Clin. Microbiol., v. 35, p. 2845-2858.
Hendolin, P. H., L. Paulin, and J. Ylikoski, 2000, Clinically applicable multiplex PCR for middle ear pathogens: J. Clin. Microbiol., v. 38 p.125-132.
Hemandez-Sanchez et al., “Lambda bar minigene-mediated inhibition of protein synthesis involves accumulation of peptidyl-tRNA and starvation for tRNA,” EMBO Jour. 17(13), 3758-65, 1998.
Hitzeman et al., Isolation and characterization of the yeast 3-phosphoglycerokinase gene (PGK) by an immunological screening technique, Journal of Biol. Chem., 255(24): 12073-80, 1980
Hogan, “Manipulating the Mouse Embryo”, Cold Spring Harbor Laboratory Press, Cold Spring Harbor, N.Y., 1986.
Howell, E. and D. Lane “Antibodies” A Laboratory Manual, Cold Spring Harbor Laboratoty, 1988.
Inoue et al, FEBS Lett. 215:327-330, 1987(a).
Inoue et al., Nucleic Acids Res. 15:6131-6148, 1987(b)
Isberg, R. R., and G. Tran Van Nhieu, 1994, Binding and internalization of microorganisms by integrin receptors: Trends in Microbiology, v.2, p.10-4, 1994.
Iwabuchi et al., Oncogene 8:1693-1696, 1993.
Johnson et al., 1993 (p. 640)
Jones, 1977
Kaufman et al., EMBO J 6:187-195, 1987.
Kessel and Gruss, Science, 249:3 74-379, 1990.
Kingsman et al., Replication in Saccharomyces cerevisiae of plasmid pBR313 carrying DNA from the yeast trpl region. Gene. Oct;7(2):141-52, 1979.
Klein, J. O., Otitis media: Clin. Infect. Dis., v. 19, p 823-833 (1994).
Koebnik, “Proposal for a peptidoglycan-associating alpha-helical motif in the C-terminal regions of some bacterial cell-surface proteins,: Mol. Microbiol. 16(6):1269-70, 1995.,
Kurjian and Herskowitz, Cell 933-943, 1982.
Kyte and Doolittle, A simple method for displaying the hydropathic character of a protein, J. Mol. Biol., 157:105-132, 1982.
Laemmli, “Cleavage of structural proteins during the assembly of the head of bacteriophage T4,” Nature (London) 227:680-685, 1970.
Lakso et al., PNAS 89:6232-6236, 1992
Lennette, “General principles for laboratory diagnosis of viral, rickettsial, and chlamydial infections,: p. 17-18, diagnostic procedures for viral, rickettsial, and chlamydial infections, vol. 7^{th} edition, 1995.
Lewis, “Programmed death in bacteria,” Microbiol. Mol. Biol. Rev. 64(3):503-14, 2000.
Li et al., Cell 69:915, 1992.
Linder, Clin. Chem. 43(2):254-266, 1997.
Lipshutz et al., Using oligonucleotide probe arrays to access genetic diversity, Biotechniques, 19(3):442-447, 1995.
Livache et al, Preparation of a DNA matrix via an electrochemically directed copolymerization of pyrrole and oligonucleotides bearing a pyrrole group, Nucleic Acids Research. 11;22(15):2915-21, 1994
Lockhart et al., Expression monitoring by hybridization to high-density oligonucleotide arrays, Nat. Biotechnol. 14(13):1649, 1996.
Loessner et al., “Evidence for a holin-like protein gene protein gene fully embedded out of frame in the endolysin gene of Staphylococcus aureus bacteriophage 187,: J. Bacteriol. 181(15):4452-60, 1999.
Lowery et al., ‘Protein measurement with the Folin-Phenol reagents:, J. Biol. Chem. 193:265-275, 1951.
Luckow and Summers, Virology 170:31-39, 1989.
Luckashin and Borodovsky, “GeneMark™.hmm: new solutions for gene finding,: Nuc. Acids Res. 26(4):1107-15, 1998.
Maher, Inhibition of T7 RNA polymerase initiation by triple-helical DNA complexes: a model for artificial gene repression, Biochemistry, 31(33):7587-94, 1992.
Maher, Bioassays 14(12):807-15, 1992.
Mansour et al., Nature 336:348, 1988.
Maxim and Gilbert, PNAS, 74:560, 1997.
Mazimanian et al., :Staphylococcus aureus sortase, an enzyme that anchors surface proteins to the cell wall,: Science 285(5428):760-3, 1999.
McAtee et al., “Characterization of a Helicobacter pylori vaccine candidate by proteome techniques,: J. Chromatogr. B. Biomed. Sci. Appl. 714(2)325-33, 1998(c).
McAtee, C. P., K. E. Fry, and D. E. Berg, 1998a Identification of potential diagnostic and vaccine candidates of Helicobacter pylori by “proteome” technologies: Helicobacter, v. 3, p. 163-9/
McAtee C. P., M. Y. Lim, K. Fung, M. Velligan, K. Fry, T. Chow, and D. E. Berg, 1998b, Identification of potential diagnostic and vaccine candidates of Helicobater pylori by two-dimensional gel electrophoresis, sequence analysis, and serum profiling: Clin. Diagn. Lab Immunol, v.5, p. 537-42
McDaniel et al., “Monoclonal antibodies against protease-sensitive pneumococcal antigens can protect mice from fatal infection with Streptococcus pneumoniae,: J. Exp. Med. 160(2):386-97, 1984.
Mejihede et al., “Ribosomal-1 frame shifting during decoding of Bacillus subtilis cdd occurs at the sequence CGA MG,” J. Bacteriol. 181(9):2930-7, 1999.
Merel, p. 1994
Morrison et al., “Isolation and characterization of three new classes of transformation deficient mutants of Streptococcus pneumoniae that are defective in DNA transport and genetic recombination,: Journal of Bacteriology, 156:281-290, 1983.
Morin et al., Nucleic Acids Res., 21:2157-2163, 1993.
Myers et al., Nature 313:495, 1985(a).
Myers et al., Science 230:1242, 1985(b).
Nabors et al., “Immunization of health adults with a single recombinant pneumococcal surface protein A (PspA) variant stimulates broadly cross-reactive antibodies to heterologous PspA molecules,” Vaccine 18:1743-1754m 2000.
Nakai and Kanehisa, “Expert system for predicting protein localization sites in gram-negative bacteria,: Proteins 11(2) 95-110, 1991.
Navarre and Schneewind, “Surface Proteins of Gram-Positive Bacteria and Mechanisms of Their Targeting to the Cell Wall Envelope,: Microbiol. Mol. Biol. Rev. 63(1):174-229, 1999.
Nielsen et al., Science251:1351-1355, 1997.
O'Gon-nan et al., Science 251:1351-1355, 1991.
Olmsted et al., “High-resolution visualization by field emission scanning electron microscopy of Enterococcus faecalis surface proteins encoded by the pheromone-inducible conjugative plasmid pCF10.” J. Bacteriol. 175(19):6229-37, 1993.
Orita et al., “Peritoneal culture alters Streptococcus pneumoniae protein profiles and virulence properties,” Infect. Immun. 68:6082-6086, 2000.
Orihuela et al., ‘Pertoneal culture alters Streptococcus pneumoniae protein profiles and virulence properties,: Infect. Immun. 68:6082-6086, 2000.
Park and Teichmann, “DIVCLUS: an automatic method in the GEANFAMMER package that finds homologous domains in single- and multi-domain proteins,: Biofomatics 14(2):144-50, 1998.
Parkhill et al., “Complete DNA sequence of a serogroup A strain of Neisseria meningitidis Z2491,: Nature 404(6777):502-6, 2000.
Pierschbacher and Ruoslahti, “Influence of stereochemistry of the sequence Arg-Gly-Asp-Xaa on binding specificity in cell adhesion,: J. Biol. Chem. 262(36):17294-8, 1987.
Pinkert, An albumin enhancer located 10 kb upstream functions along with its promoter to direct efficient, liver-specific expression in transgenic mice, Genes & Development. (3): 268-76, 1987.
Pizza et al., “Identification of vaccine candidates against serogroup B meningococcus by whole-genome sequencing,: Science 287(5459):1816-20, 2000.
Pugsley, “The complete general secretory pathway in gram-negative bacteria,: Microbiol. Rev. 57(1):50-108, 1993.
Queen and Baltimore, Immunoglobulin gene transcription is activated by downstream sequence elements, Cell, 33:741-748, 1983.
Raham et al., Journal of Neuroscience 19:2016-2026, 1999.
Ranande, V. V., Drug delivery systems. 1. site-specific drug delivery using liposomes as carriers, Journal of Clinical Pharmacology,29(8):685-94, 1989.
Rose et al., “Methods in Yeast Genetics: A Laboratory Course Manual.: Cold Harbor Press, Cold Spring Harbor, N.Y. (1990).
Rosenow et al., “Contribution of novel choline-binding proteins to adherence, colonization and immunogenicity of Streptococcus pneumoniae,: Mol. Microbiol. 25(5):819-29, 1997.
Ross and Wilkie, “GTP'ase-activating proteins for Heterotrimeric G proteins: Regulators of G protein Signaling (RGS) and RGS-like proteins,: Annual Review of Biochemistry 69:795-827, 2000.
Saleeba et al., Meth. Enzymol. 217:286-295, 1992.
Salzberg et al., “Microbial gene identification using interpolated Markov models,: Nuc. Acids. Res. 26(2):544-8, 1998.
Sambrook et al., “Molecular Cloning: A Laboratory Manual: 2^{nd}, ed., Cold Spring Harbor Laboratory, Cold Spring Harbor Laboratory Press, Cold Spring Harbor, N.Y. 1989.
Sampson et al., :Cloning and nucleotide sequence analysis of psaa, the Streptococcus pneumoniae gene encoding a 37-kilodalton protein homologous to previously reported Streptococcus Sp. Adhesins,: Infect Immun. 62(1):319-24, 1994.
Sanger, PNAS 74:5463, 1977.
Schappert, S., 1991, Office Visits for Otitis Media: United States, 1975-1990: DHHS publication No. PHS 92-1250; Hyattsville, Md., US Dept. of Health and Human Sciences, Public Health Services, Centers for Disease Control and Prevention, National Center Schultz et al., Gene 54:113-123, 1987
Schultz, 1987 (p.646).
Seed, B., An LFA-3 cDNA encodes a phospholipid-linked membrane protein homologous to its receptor CD2, Nature, 329(6142):840-842, 1987.
Seed B. and Aruffo A, Molecular cloning of the CD2 antigen, the T-cell erythrocyte receptor, by a rapid immunoselection procedure, Proc. Natl. Acad. Sciences U.S.A. 1987 Sebwenlist et al., 1980
E. L. Shledon, 1993
Shinefield and Black, “Efficacy of pneumococcal conjugate vaccines in large scale field trials (In Process Citation), “Pediatr. Infect Dis. J. 19(4):394-7, 2000
Shoemaker et al., 1996
Sih, T., B. Schwartz, G. Bosley, and R. Facklam, 1992, Chronic otitis media with effusion caused by Alloiococcus otitis: clinical and laboratory features: the 32^{nd} lnterscience Conference on Antimicrobial Agents and Chemotherapy, p. 222.
Simon et al., Science, 252:802-8, 1991.
Smith and Johnson, Gene 67:31-40, 1988.
Smith et al., Mol. Cell Biol. 3:2156-2165, 1983.
Smith, T. L. and Waterman, M. S. Waterman, Identification of common molecular subsequences. J. Mol. Biol, 147,195-197 (1981).
Songyang, et al., Cell72:767-778, 1993.
Sonhammer, E. L.,G. von Heijne, and A Krogh, 1988, A hidden Markov model for predicting transmembrane helices in protein sequences: Ismb, v. 6, p.175-82.
Sonnhammer, E. L..,S. R. Eddy, and R. Durbin, 1997 Pfam: a comprehensive database of protein domain families based on seed alignments: Proteins, v. 28, p. 405-20.
Stinchcomb et al., 1979
Stratford, Perricaudet, et al., 1992.
Sonnenberg and Belisle, “Definition of Mycobacterium tuberculosis culture filtrate proteins by two-dimensional polyacrylamide gel electrophoresis, N-terminal amino acid sequencing, and electrospray mass spectrometry,” Infect. Immun. 65(11):4515-24, 1997. Sosnowsky et al., 1997.
Stockbauer et al., “A natural variant of the cysteine protease virulence factor of group A streptQcoccus with an arginine-glycine-aspartic acid (RGD) motif preferentially binds human integrins alphavbeta3 and alphallbbeta3 (In Process Citation),: Proc. Natl. Acad. Sci. USA 96(1):242-7, 1999.
Studier et al., “Protection of mice against fata pneumococcal challenge by immunization with pneumococcal surface adhesin A (PsaA),: Microb. Pathog. 21(1):17-22, 1996
Studier et al., Use of T7 RNA polymerase to direct expression of cloned genes, Methods in Enzymology,185:60-89, 1990.
Talkington et al., “Protection of mice against fatal pneumococcal challenge by immunization with pneumococcal surface adhesin A (PsaA),: Microb. Pathog. 21(1):17-22, 1996.
Tebbey et al., “Effective mucosal immunization against respiratory syncytial virus using a genetically detoxified cholera holotoxin, CT-E29H,” Vaccine 18(24):2723-34, 2000.
Thomas and Capecchi, Cell 51:503, 1987
Weldingh et al., “Two-dimensional electrophoresis for analysis of Mycobacterium tuberculosis culture filtrate and purification and characterization of six novel proteins,” Infect. Immun. 66(8):3492-500, 1998.
Wilmut et al., Nature 385:810-813, 1997.
Wilson et al., Cell 37-767, 1984.
Winoto and Baltimore, A novel, inducible and T cell-specific enhancer located at the 3′ end of the T cell receptor alpha locus, EMBO Journal. 8(3):729-733, 1989.
Xu et al., “PHR1 encodes an abundant, pleckstrin homology domain-containing Integral membrane protein in the photoreceptor outer segments,: Journal of Biological Chemistry 274:35676-35685, 1999.
Yamamoto et al., “A nontoxic adjuvant for mucosal immunity to peumococcal surface protein,: A. J. Immune. 161(8):4115-21, 1998.
Yershov et al., DNA analysis and diagnostics on oligonucleotide microchips, Proc. Natl. Acad. Sciences U.S.A.93(10):4913-8, 1996.
Servos et al., Cell 72:223-232, 1993.
Zhang et al., 2001, “Recombinant PhpA Protein, a Unique Histidine Motif-Containing Protein from Streptococcus pneumoniae, Protects Mice against Intranasal Pneumococcal Challenge,: Infect. Immun. 69:3827-3836

1. An isolated polynucleotide of an Alloiococcus otitidis genomic sequence, wherein the polynucleotide comprises a nucleotide sequence having at least about 70% identity to a nucleotide sequence chosen from one of odd numbered sequences from SEQ ID NO: 1 to SEQ ID NO: 6649, a complement thereof, a degenerate variant thereof, and a fragment thereof.

2. The polynucleotide of claim 1, wherein the polynucleotide is a complement to a nucleotide sequence chosen from one of odd numbered sequences from SEQ ID NO: 1 to SEQ ID NO: 6649, a complement thereof, a degenerate variant thereof, and a fragment thereof.

3. The polynucleotide of claim 2, wherein the polynucleotide is selected from the group consisting of genomic DNA, cDNA and RNA.

4. The polynucleotide of claim 3, wherein the polynucleotide further comprises heterologous nucleotides.

5. An isolated polynucleotide which hybridizes to a nucleotide sequence chosen from one of the odd numbered sequences from SEQ ID NO: 1 to SEQ ID NO: 6649, a complement thereof, a degenerate variant thereof, or a fragment thereof, under high stringency hybridization conditions.

6. The polynucleotide of claim 5, wherein the polynucleotide hybridizes under intermediate stringency hybridization conditions.

7. An isolated polynucleotide of an Alloiococcus otitidis genomic sequence, wherein the polynucleotide comprises a nucleotide sequence chosen from one of odd numbered sequences from SEQ ID NO: 1 to SEQ ID NO: 6649, a complement thereof, a fragment thereof, or a degenerate variant thereof, and encodes a polypeptide, a biological equivalent thereof, or a fragment thereof, and is selected from the group consisting of:
(a) an Alloiococcus otitidis polypeptide predicted by SignalP algorithm analysis as having met all four criteria set out in the SignalP algorithm for a polypeptide having a signal peptide and predicted by HMM SignalP algorithm analysis as having a signal peptide;
(b) an Alloiococcus otitidis polypeptide predicted by SignalP algorithm analysis as having met three of the four criteria set out in the SignalP algorithm for a polypeptide having a signal peptide and predicted by HMM SignalP algorithm analysis as having a signal peptide;
(c) an Alloiococcus otitidis polypeptide predicted by HMM Signal P algorithm analysis as having a signal peptide;
(d) an Alloiococcus otitidis polypeptide predicted by HMM Signal P algorithm analysis as being a non-secretory protein and by PSORT program to be localized to the cytoplasmic membrane;
(e) an Alloiococcus otitidis polypeptide identified by BlastP analysis;
(f) an Alloiococcus otitidis polypeptide identified by Pfam analysis;
(g) an Alloiococcus otitidis polypeptide identified using proteomics methodology as sharing homology with surface exposed proteins of Streptococcus pneumoniae;
(h) an Alloiococcus otitidis lipoprotein;
(i) an Alloiococcus otitidis polypeptide having a LPXTG motif, wherein the polypeptide is covalently attached to the peptidoglycan layer;
(j) an Alloiococcus otitidis polypeptide, wherein the polypeptide is non-covalently associated with the peptidoglycan layer;
(k) an Alloiococcus otitidis polypeptide having an RGDX motif wherein X is not a proline residue;
(l) an Alloiococcus otitidis polypeptide predicted by BlastP as being involved in capsule biosynthesis and transport;
(m) an Alloiococcus otitidis polypeptide identified by BlastP as being localized within the capsule loci of the Alloiococcus otitidis genome;
(n) an Alloiococcus otitidis polypeptide predicted by BlastP as being associated with sporulation;
(o) an Alloiococcus otitidis polypeptide encoded by unique ORFs identified by BlastP analysis as having a BlastP ‘E Value’ of >e⁻¹⁰;
(p) an Alloiococcus otitidis polypeptide identified by Glimmer™ ORF finder program;
(q) an Alloiococcus otitidis polypeptide identified by GeneMark™ ORF finder program;
(r) an Alloiococcus otitidis polypeptide identified by an ORF finder program that searches for an ATG, GTG or TTG Start codon between a Stop-Stop region; and
(s) an Alloiococcus otitidis polypeptide identified by an ORF finder program that searches for a transmembrane domain between two Stop codons and a Start codon immediately upstream of this transmembrane region.

8. The polynucleotide of claim 7, wherein the polynucleotide is a complement to a nucleotide sequence selected from one of the odd numbered sequences from SEQ ID NO: 1 to SEQ ID NO: 6649, a degenerate variant thereof, and a fragment thereof.

9. The polynucleotide of claim 8, wherein the polynucleotide is selected from the group consisting of genomic DNA, cDNA and RNA.

10. The polynucleotide of claim 9, wherein the polynucleotide further comprises heterologous nucleotides.

11. The polynucleotide of claim 10, wherein the polynucleotide encodes a fusion polypeptide.

12. The polynucleotide of claim 7 wherein the polynucleotide encodes a polypeptide, which is predicted by SignalP algorithm analysis as having met all four criteria set out in the SignalP algorithm for a polypeptide having a signal peptide, and which is predicted by HMM SignalP algorithm analysis as having a signal peptide, and the polynucleotide is selected from one of Seq. ID No. 89, Seq. ID No. 127, Seq. ID No. 131, Seq. ID No. 133, Seq. ID No. 161, Seq. ID No. 163, Seq. ID No. 213, Seq. ID No. 215, Seq. ID No. 225, Seq. ID No. 249, Seq. ID No. 251, Seq. ID No. 287, Seq. ID No. 289, Seq. ID No. 369, Seq. ID No. 371, Seq. ID No. 375, Seq. ID No. 377, Seq. ID No. 497, Seq. ID No. 503, Seq. ID No. 505, Seq. ID No. 527, Seq. ID No. 549, Seq. ID No. 575, Seq. ID No. 617, Seq. ID No. 657, Seq. ID No. 659, Seq. ID No. 661, Seq. ID No. 675, Seq. ID No. 715, Seq. ID No. 717, Seq. ID No. 745, Seq. ID No. 747, Seq. ID No. 755, Seq. ID No. 757, Seq. ID No. 759, Seq. ID No. 761, Seq. ID No. 773, Seq. ID No. 807, Seq. ID No. 967, Seq. ID No. 973, Seq. ID No. 975, Seq. ID No. 1013, Seq. ID No. 1025, Seq. ID No. 1027, Seq. ID No. 1039, Seq. ID No. 1075, Seq. ID No. 1089, Seq. ID No. 1195, Seq. ID No. 1197, Seq. ID No. 1207, Seq. ID No. 1239, Seq. ID No. 1243, Seq. ID No. 1245, Seq. ID No. 1269, Seq. ID No. 1315, Seq. ID No. 1317, Seq. ID No. 1331, Seq. ID No. 1365, Seq. ID No. 1367, Seq. ID No. 1369, Seq. ID No. 1445, Seq. ID No. 1447, Seq. ID No. 1513, Seq. ID No. 1517, Seq. ID No. 1545, Seq. ID No. 1549, Seq. ID No. 1561, Seq. ID No. 1569, Seq. ID No. 1571, Seq. ID No. 1585, Seq. ID No. 1643, Seq. ID No. 1653, Seq. ID No. 1661, Seq. ID No. 1663, Seq. ID No. 1707, Seq. ID No. 1733, Seq. ID No. 1737, Seq. ID No. 1755, Seq. ID No. 1765, Seq. ID No. 1767, Seq. ID No. 1775, Seq. ID No. 1791, Seq. ID No. 1805, Seq. ID No. 1875, Seq. ID No. 1969, Seq. ID No. 1981, Seq. ID No. 2053, Seq. ID No. 2055, Seq. ID No. 2087, Seq. ID No. 2089, Seq. ID No. 2091, Seq. ID No. 2105, Seq. ID No. 2115, Seq. ID No. 2149, Seq. ID No. 2151, Seq. ID No. 2159, Seq. ID No. 2177, Seq. ID No. 2277, Seq. ID No. 2293, Seq. ID No. 2321, Seq. ID No. 2347, Seq. ID No. 2351, Seq. ID No. 2353, Seq. ID No. 2365, Seq. ID No. 2395, Seq. ID No. 2421, Seq. ID No. 2425, Seq. ID No. 2427, Seq. ID No. 2429, Seq. ID No. 2449, Seq. ID No. 2459, Seq. ID No. 2525, Seq. ID No. 2531, Seq. ID No. 2607, Seq. ID No. 2651, Seq. ID No. 2679, Seq. ID No. 2693, Seq. ID No. 2699, Seq. ID No. 2703, Seq. ID No. 2753, Seq. ID No. 2785, Seq. ID No. 2813, Seq. ID No. 2829, Seq. ID No. 2835, Seq. ID No 2841, Seq. ID No. 2879, Seq. ID No. 2881, Seq. ID No. 2935, Seq. ID No. 2937, Seq. ID No. 2949, Seq. ID No. 2965, Seq. ID No. 2967, Seq. ID No. 2973, Seq. ID No. 3007, Seq. ID No. 3009, Seq. ID No. 3011, Seq. ID No. 3029, Seq. ID No. 3031, Seq. ID No. 3109, Seq. ID No. 3111, Seq. ID No. 3219, Seq. ID No. 3223, Seq. ID No. 3273, Seq. ID No. 3297, Seq. ID No. 3331, Seq. ID No. 3335, Seq. ID No. 3341, Seq. ID No. 3349, Seq. ID No. 3353, Seq. ID No. 3355, Seq. ID No. 3377, Seq. ID No. 3391, Seq. ID No. 3425, Seq. ID No. 3449, Seq. ID No. 3493, Seq. ID No. 3573, Seq. ID No. 3611, Seq. ID No. 3619, Seq. ID No. 3689, Seq. ID No. 3753, Seq. ID No. 3755, Seq. ID No. 3765, Seq. ID No. 3781, Seq. ID No. 3783, Seq. ID No. 3851, Seq. ID No. 3903, Seq. ID No. 4005, Seq. ID No. 4007, Seq. ID No. 4027, Seq. ID No. 4073, Seq. ID No. 4137, Seq. ID No. 4141, Seq. ID No. 4143, Seq. ID No. 4145, Seq. ID No. 4225, Seq. ID No. 4343, Seq. ID No. 4385, Seq. ID No. 4399, Seq. ID No. 4469, Seq. ID No. 4483, Seq. ID No 4493, Seq. ID No. 4495, Seq. ID No. 4533, Seq. ID No. 4699, Seq. ID No. 4709, Seq. ID No. 4711, Seq. ID No. 4735, Seq. ID No. 4827, Seq. ID No. 4871, Seq. ID No. 4873, Seq. ID No. 4879, Seq. ID No. 4921, Seq. ID No. 4981, Seq. ID No. 5115, Seq. ID No. 5267, Seq. ID No. 5341, Seq. ID No. 5387, Seq. ID No. 5465, Seq. ID No. 5467, Seq. ID No. 5479, Seq. ID No. 5497, Seq. ID No. 5513, Seq. ID No. 5543, Seq. ID No. 5545, Seq. ID No. 5567, Seq. ID No. 5587, Seq. ID No. 5589, Seq. ID No. 5591, Seq. ID No. 5611, Seq. ID No. 5621, Seq. ID No. 5641, Seq. ID No. 5681, Seq. ID No. 5719, Seq. ID No. 5771, Seq. ID No. 5783, Seq. ID No. 5895, Seq. ID No. 5899, Seq. ID No. 5901, Seq. ID No. 5927, Seq. ID No. 5929, Seq. ID No. 5939, Seq. ID No. 5987, Seq. ID No. 6013, Seq. ID No. 6087, Seq. ID No. 6089, Seq. ID No. 6091, Seq. ID No. 6093, Seq. ID No. 6095, Seq. ID No. 6103, Seq. ID No. 6169, Seq. ID No. 6185, Seq. ID No. 6187, Seq. ID No. 6195, Seq. ID No. 6197, Seq. ID No. 6203, Seq. ID No. 6205, Seq. ID No. 6221, Seq. ID No. 6223, Seq. ID No. 6229, Seq. ID No. 6291, Seq. ID No. 6415, Seq. ID No. 6417, Seq. ID No. 6473, Seq. ID No. 6507, Seq. ID No. 6543, Seq. ID No. 6595, Seq. ID No. 6597, Seq. ID No. 6599, Seq. ID No. 6601 and Seq. ID No. 6603.

13. The polynucleotide of claim 7 wherein the polynucleotide encodes a polypeptide, which is predicted by SignalP algorithm analysis as having met three of the four criteria set out in the SignalP algorithm for a polypeptide having a signal peptide and which is predicted by HMM SignalP algorithm analysis as having a signal peptide, and the polynucleotide is selected from one of Seq. ID No. 7, Seq. ID No. 11, Seq. ID No. 23, Seq. ID No. 45, Seq. ID No. 47, Seq. ID No. 53, Seq. ID No. 59, Seq. ID No. 65, Seq. ID No. 67, Seq. ID No. 87, Seq. ID No. 91, Seq. ID No. 139, Seq. ID No. 151, Seq. ID No. 159, Seq. ID No. 165, Seq. ID No. 169, Seq. ID No. 185, Seq. ID No. 211, Seq. ID No. 217, Seq. ID No. 235, Seq. ID No. 237, Seq. ID No. 239, Seq. ID No. 271, Seq. ID No. 285, Seq. ID No. 291, Seq. ID No. 327, Seq. ID No. 349, Seq. ID No. 385, Seq. ID No. 387, Seq. ID No. 407, Seq. ID No. 427, Seq. ID No. 431, Seq. ID No. 455, Seq. ID No. 457, Seq. ID No. 495, Seq. ID No. 499, Seq. ID No. 511, Seq. ID No. 513, Seq. ID No. 515, Seq. ID No. 525, Seq. ID No. 545, Seq. ID No. 547, Seq. ID No. 557, Seq. ID No. 569, Seq. ID No. 589, Seq. ID No. 591, Seq. ID No. 593, Seq. ID No. 595, Seq. ID No. 609, Seq. ID No. 637, Seq. ID No. 639, Seq. ID No. 641, Seq. ID No. 653, Seq. ID No. 669, Seq. ID No. 679, Seq. ID No. 719, Seq. ID No. 723, Seq. ID No. 739, Seq. ID No. 749, Seq. ID No. 751, Seq. ID No. 763, Seq. ID No. 765, Seq. ID No. 769, Seq. ID No. 777, Seq. ID No. 779, Seq. ID No. 781, Seq. ID No. 819, Seq. ID No. 895, Seq. ID No. 903, Seq. ID No. 905, Seq. ID No. 909, Seq. ID No. 935, Seq. ID No. 937, Seq. ID No. 951, Seq. ID No. 955, Seq. ID No. 979, Seq. ID No. 981, Seq. ID No. 983, Seq. ID No. 985, Seq. ID No. 1005, Seq. ID No. 1007, Seq. ID No. 1009, Seq. ID No. 1015, Seq. ID No. 1019, Seq. ID No. 1029, Seq. ID No. 1031, Seq. ID No. 1047, Seq. ID No. 1091, Seq. ID No. 1111, Seq. ID No. 1137, Seq. ID No. 1151, Seq. ID No. 1153, Seq. ID No. 1169, Seq. ID No. 1173, Seq. ID No. 1183, Seq. ID No. 1191, Seq. ID No. 1193, Seq. ID No. 1213, Seq. ID No. 1223, Seq. ID No. 1229, Seq. ID No. 1247, Seq. ID No. 1259, Seq. ID No. 1283, Seq. ID No. 1307, Seq. ID No. 1319, Seq. ID No. 1321, Seq. ID No. 1327, Seq. ID No. 1341, Seq. ID No. 1343, Seq. ID No. 1347, Seq. ID No. 1389, Seq. ID No. 1401, Seq. ID No. 1429, Seq. ID No. 1479, Seq. ID No. 1551, Seq. ID No. 1559, Seq. ID No. 1579, Seq. ID No. 1587, Seq. ID No. 1621, Seq. ID No. 1627, Seq. ID No. 1633, Seq. ID No. 1645, Seq. ID No. 1647, Seq. ID No. 1649, Seq. ID No. 1655, Seq. ID No. 1665, Seq. ID No. 1667, Seq. ID No. 1671, Seq. ID No. 1677, Seq. ID No. 1679, Seq. ID No. 1681, Seq. ID No. 1695, Seq. ID No. 1703, Seq. ID No. 1705, Seq. ID No. 1731, Seq. ID No. 1753, Seq. ID No. 1759, Seq. ID No. 1763, Seq. ID No. 1787, Seq. ID No. 1797, Seq. ID No. 1821, Seq. ID No. 1823, Seq. ID No. 1833, Seq. ID No. 1839, Seq. ID No. 1841, Seq. ID No. 1843, Seq. ID No. 1893, Seq. ID No. 1917, Seq. ID No. 1919, Seq. ID No. 1971, Seq. ID No. 1991, Seq. ID No. 2013, Seq. ID No. 2015, Seq. ID No. 2017, Seq. ID No. 2019, Seq. ID No. 2033, Seq. ID No. 2035, Seq. ID No. 2049, Seq. ID No. 2081, Seq. ID No. 2083, Seq. ID No. 2085, Seq. ID No. 2111, Seq. ID No. 2113, Seq. ID No. 2123, Seq. ID No. 2125, Seq. ID No. 2127, Seq. ID No. 2131, Seq. ID No. 2133, Seq. ID No. 2153, Seq. ID No. 2161, Seq. ID No. 2171, Seq. ID No. 2175, Seq. ID No. 2179, Seq. ID No. 2187, Seq. ID No. 2201, Seq. ID No. 2203, Seq. ID No. 2221, Seq. ID No. 2237, Seq. ID No. 2249, Seq. ID No. 2251, Seq. ID No. 2255, Seq. ID No. 2261, Seq. ID No. 2263, Seq. ID No. 2271, Seq. ID No. 2279, Seq. ID No. 2283, Seq. ID No. 2285, Seq. ID No. 2289, Seq. ID No. 2295, Seq. ID No. 2305, Seq. ID No. 2307, Seq. ID No. 2317, Seq. ID No. 2319, Seq. ID No. 2323, Seq. ID No. 2325, Seq. ID No. 2343, Seq. ID No. 2349, Seq. ID No. 2385, Seq. ID No. 2393, Seq. ID No. 2423, Seq. ID No. 2453, Seq. ID No. 2491, Seq. ID No. 2497, Seq. ID No. 2499, Seq. ID No. 2507, Seq. ID No. 2515, Seq. ID No. 2517, Seq. ID No. 2521, Seq. ID No. 2523, Seq. ID No. 2527, Seq. ID No. 2533, Seq. ID No. 2535, Seq. ID No. 2541, Seq. ID No. 2543, Seq. ID No. 2547, Seq. ID No. 2549, Seq. ID No. 2557, Seq. ID No. 2573, Seq. ID No. 2583, Seq. ID No. 2601, Seq. ID No. 2603, Seq. ID No. 2621, Seq. ID No. 2653, Seq. ID No. 2681, Seq. ID No. 2691, Seq. ID No. 2701, Seq. ID No. 2733, Seq. ID No. 2739, Seq. ID No. 2801, Seq. ID No. 2807, Seq. ID No. 2815, Seq. ID No. 2819, Seq. ID No. 2821, Seq. ID No. 2823, Seq. ID No. 2833, Seq. ID No. 2837, Seq. ID No. 2839, Seq. ID No. 2845, Seq. ID No. 2853, Seq. ID No. 2861, Seq. ID No. 2865, Seq. ID No. 2873, Seq. ID No. 2875, Seq. ID No. 2877, Seq. ID No. 2883, Seq. ID No. 2895, Seq. ID No. 2905, Seq. ID No. 2939, Seq. ID No. 2941, Seq. ID No. 2947, Seq. ID No. 2981, Seq. ID No. 2983, Seq. ID No. 2987, Seq. ID No. 2991, Seq. ID No. 2995, Seq. ID No. 3001, Seq. ID No. 3005, Seq. ID No. 3021, Seq. ID No. 3025, Seq. ID No. 3043, Seq. ID No. 3045, Seq. ID No. 3073, Seq. ID No. 3075, Seq. ID No. 3155, Seq. ID No. 3163, Seq. ID No. 3165, Seq. ID No. 3167, Seq. ID No. 3183, Seq. ID No. 3213, Seq. ID No. 3215, Seq. ID No. 3235, Seq. ID No. 3245, Seq. ID No. 3261, Seq. ID No. 3263, Seq. ID No. 3265, Seq. ID No. 3269, Seq. ID No. 3271, Seq. ID No. 3275, Seq. ID No. 3277, Seq. ID No. 3317, Seq. ID No. 3325, Seq. ID No. 3337, Seq. ID No. 3351, Seq. ID No. 3389, Seq. ID No. 3405, Seq. ID No. 3419, Seq. ID No. 3421, Seq. ID No. 3423, Seq. ID No. 3451, Seq. ID No. 3453, Seq. ID No. 3455, Seq. ID No. 3491, Seq. ID No. 3495, Seq. ID No. 3503, Seq. ID No. 3505, Seq. ID No. 3517, Seq. ID No. 3519, Seq. ID No. 3545, Seq. ID No. 3547, Seq. ID No. 3571, Seq. ID No. 3593, Seq. ID No. 3617, Seq. ID No. 3621, Seq. ID No. 3629, Seq. ID No. 3663, Seq. ID No. 3701, Seq. ID No. 3703, Seq. ID No. 3723, Seq. ID No. 3737, Seq. ID No. 3741, Seq. ID No. 3745, Seq. ID No. 3747, Seq. ID No. 3759, Seq. ID No. 3763, Seq. ID No. 3767, Seq. ID No. 3769, Seq. ID No. 3771, Seq. ID No. 3785, Seq. ID No. 3793, Seq. ID No. 3813, Seq. ID No. 3837, Seq. ID No. 3839, Seq. ID No. 3861, Seq. ID No. 3865, Seq. ID No. 3889, Seq. ID No. 3891, Seq. ID No. 3895, Seq. ID No. 3915, Seq. ID No. 3923, Seq. ID No. 3925, Seq. ID No. 3929, Seq. ID No. 3965, Seq. ID No. 3975, Seq. ID No. 3979, Seq. ID No. 3995, Seq. ID No. 3997, Seq. ID No. 4009, Seq. ID No. 4039, Seq. ID No. 4041, Seq. ID No. 4061, Seq. ID No. 4079, Seq. ID No. 4081, Seq. ID No. 4111, Seq. ID No. 4113, Seq. ID No. 4135, Seq. ID No. 4139, Seq. ID No. 4155, Seq. ID No. 4181, Seq. ID No. 4203, Seq. ID No. 4221, Seq. ID No. 4247, Seq. ID No. 4253, Seq. ID No. 4257, Seq. ID No. 4259, Seq. ID No. 4261, Seq. ID No. 4263, Seq. ID No. 4269, Seq. ID No. 4311, Seq. ID No. 4379, Seq. ID No. 4389, Seq. ID No. 4391, Seq. ID No. 4471, Seq. ID No. 4473, Seq. ID No. 4481, Seq. ID No. 4525, Seq. ID No. 4551, Seq. ID No. 4561, Seq. ID No. 4573, Seq. ID No. 4617, Seq. ID No. 4639, Seq. ID No. 4659, Seq. ID No. 4681, Seq. ID No. 4701, Seq. ID No. 4705, Seq. ID No. 4713, Seq. ID No. 4717, Seq. ID No. 4721, Seq. ID No. 4737, Seq. ID No. 4741, Seq. ID No. 4813, Seq. ID No. 4817, Seq. ID No. 4819, Seq. ID No. 4829, Seq. ID No. 4831, Seq. ID No. 4851, Seq. ID No. 4869, Seq. ID No. 4875, Seq. ID No. 4913, Seq. ID No. 4919, Seq. ID No. 4929, Seq. ID No. 4931, Seq. ID No. 4983, Seq. ID No. 4993, Seq. ID No. 5011, Seq. ID No. 5023, Seq. ID No. 5031, Seq. ID No. 5033, Seq. ID No. 5035, Seq. ID No. 5043, Seq. ID No. 5061, Seq. ID No. 5063, Seq. ID No. 5069, Seq. ID No. 5103, Seq. ID No. 5121, Seq. ID No. 5123, Seq. ID No. 5131, Seq. ID No. 5133, Seq. ID No. 5135, Seq. ID No. 5137, Seq. ID No. 5139, Seq. ID No. 5141, Seq. ID No. 5151, Seq. ID No. 5159, Seq. ID No. 5161, Seq. ID No. 5165, Seq. ID No. 5167, Seq. ID No. 5175, Seq. ID No. 5177, Seq. ID No. 5205, Seq. ID No. 5207, Seq. ID No. 5211, Seq. ID No. 5215, Seq. ID No. 5219, Seq. ID No. 5223, Seq. ID No. 5285, Seq. ID No. 5287, Seq. ID No. 5329, Seq. ID No. 5331, Seq. ID No. 5377, Seq. ID No. 5379, Seq. ID No. 5381, Seq. ID No. 5385, Seq. ID No. 5405, Seq. ID No. 5407, Seq. ID No. 5453, Seq. ID No. 5459, Seq. ID No. 5461, Seq. ID No. 5463, Seq. ID No. 5481, Seq. ID No. 5509, Seq. ID No. 5529, Seq. ID No. 5531, Seq. ID No. 5557, Seq. ID No. 5569, Seq. ID No. 5577, Seq. ID No. 5623, Seq. ID No. 5625, Seq. ID No. 5643, Seq. ID No. 5659, Seq. ID No. 5661, Seq. ID No. 5683, Seq. ID No. 5685, Seq. ID No. 5709, Seq. ID No. 5711, Seq. ID No. 5733, Seq. ID No. 5739, Seq. ID No. 5765, Seq. ID No. 5797, Seq. ID No. 5827, Seq. ID No. 5889, Seq. ID No. 5893, Seq. ID No. 5905, Seq. ID No. 5909, Seq. ID No. 5911, Seq. ID No. 5923, Seq. ID No. 5963, Seq. ID No. 5965, Seq. ID No. 6025, Seq. ID No. 6027, Seq. ID No. 6031, Seq. ID No. 6043, Seq. ID No. 6045, Seq. ID No. 6047, Seq. ID No. 6053, Seq. ID No. 6059, Seq. ID No. 6109, Seq. ID No. 6111, Seq. ID No. 6115, Seq. ID No. 6117, Seq. ID No. 6127, Seq. ID No. 6131, Seq. ID No. 6135, Seq. ID No. 6137, Seq. ID No. 6161, Seq. ID No. 6165, Seq. ID No. 6183, Seq. ID No. 6189, Seq. ID No. 6191, Seq. ID No. 6193, Seq. ID No. 6207, Seq. ID No. 6209, Seq. ID No. 6217, Seq. ID No. 6225, Seq. ID No. 6235, Seq. ID No. 6243, Seq. ID No. 6249, Seq. ID No. 6253, Seq. ID No. 6259, Seq. ID No. 6265, Seq. ID No. 6267, Seq. ID No. 6275, Seq. ID No. 6283, Seq. ID No. 6285, Seq. ID No. 6303, Seq. ID No. 6325, Seq. ID No. 6327, Seq. ID No. 6363, Seq. ID No. 6365, Seq. ID No. 6367, Seq. ID No. 6385, Seq. ID No. 6425, Seq. ID No. 6427, Seq. ID No. 6429, Seq. ID No. 6431, Seq. ID No. 6453, Seq. ID No. 6465, Seq. ID No. 6469, Seq. ID No. 6471, Seq. ID No. 6479, Seq. ID No. 6539, Seq. ID No. 6541, Seq. ID No. 6571, Seq. ID No. 6573, Seq. ID No. 6639, Seq. ID No. 6643, and Seq. ID No. 6645.

14. The polynucleotide of claim 7 wherein the polynucleotide encodes a polypeptide, which is predicted by HMM Signal P algorithm analysis as having a signal peptide, and the polynucleotide is selected from one of Seq. ID No. 3, Seq. ID No. 7, Seq. ID No. 9, Seq. ID No. 19, Seq. ID No. 21, Seq. ID No. 31, Seq. ID No. 33, Seq. ID No. 45, Seq. ID No. 47, Seq. ID No. 49, Seq. ID No. 53, Seq. ID No. 65, Seq. ID No. 67, Seq. ID No. 85, Seq. ID No. 87, Seq. ID No. 105, Seq. ID No. 111, Seq. ID No. 123, Seq. ID No. 131, Seq. ID No. 133, Seq. ID No. 141, Seq. ID No. 159, Seq. ID No. 161, Seq. ID No. 163, Seq. ID No. 171, Seq. ID No. 191, Seq. ID No. 209, Seq. ID No. 211, Seq. ID No. 213, Seq. ID No. 215, Seq. ID No. 217, Seq. ID No. 225, Seq. ID No. 235, Seq. ID No. 237, Seq. ID No. 249, Seq. ID No. 251, Seq. ID No. 255, Seq. ID No. 257, Seq. ID No. 259, Seq. ID No. 271, Seq. ID No. 273, Seq. ID No. 277, Seq. ID No. 285, Seq. ID No. 287, Seq. ID No. 289, Seq. ID No. 327, Seq. ID No. 329, Seq. ID No. 357, Seq. ID No. 359, Seq. ID No. 373, Seq. ID No. 375, Seq. ID No. 393, Seq. ID No. 401, Seq. ID No. 403, Seq. ID No. 455, Seq. ID No. 457, Seq. ID No. 477, Seq. ID No. 487, Seq. ID No. 489, Seq. ID No. 491, Seq. ID No. 493, Seq. ID No. 495, Seq. ID No. 497, Seq. ID No. 499, Seq. ID No. 503, Seq. ID No. 505, Seq. ID No. 509, Seq. ID No. 511, Seq. ID No. 513, Seq. ID No. 515, Seq. ID No. 525, Seq. ID No. 527, Seq. ID No. 545, Seq. ID No. 547, Seq. ID No. 549, Seq. ID No. 557, Seq. ID No. 567, Seq. ID No. 569, Seq. ID No. 575, Seq. ID No. 577, Seq. ID No. 579, Seq. ID No. 591, Seq. ID No. 593, Seq. ID No. 599, Seq. ID No. 617, Seq. ID No. 637, Seq. ID No. 639, Seq. ID No. 641, Seq. ID No. 643, Seq. ID No. 645, Seq. ID No. 647, Seq. ID No. 653, Seq. ID No. 655, Seq. ID No. 657, Seq. ID No. 659, Seq. ID No. 661, Seq. ID No. 667, Seq. ID No. 669, Seq. ID No. 675, Seq. ID No. 677, Seq. ID No. 679, Seq. ID No. 715, Seq. ID No. 717, Seq. ID No. 719, Seq. ID No. 721, Seq. ID No. 723, Seq. ID No. 725, Seq. ID No. 745, Seq. ID No. 747, Seq. ID No. 749, Seq. ID No. 751, Seq. ID No. 755, Seq. ID No. 757, Seq. ID No. 759, Seq. ID No. 761, Seq. ID No. 763, Seq. ID No. 765, Seq. ID No. 769, Seq. ID No. 777, Seq. ID No. 779, Seq. ID No. 781, Seq. ID No. 807, Seq. ID No. 809, Seq. ID No. 819, Seq. ID No. 825, Seq. ID No. 863, Seq. ID No. 893, Seq. ID No. 901, Seq. ID No. 935, Seq. ID No. 937, Seq. ID No. 941, Seq. ID No. 943, Seq. ID No. 955, Seq. ID No. 957, Seq. ID No. 967, Seq. ID No. 979, Seq. ID No. 981, Seq. ID No. 983, Seq. ID No. 985, Seq. ID No. 1001, Seq. ID No. 1003, Seq. ID No. 1005, Seq. ID No. 1007, Seq. ID No. 1009, Seq. ID No. 1011, Seq. ID No. 1013, Seq. ID No. 1015, Seq. ID No. 1019, Seq. ID No. 1029, Seq. ID No. 1031, Seq. ID No. 1033, Seq. ID No. 1035, Seq. ID No. 1037, Seq. ID No. 1039, Seq. ID No. 1045, Seq. ID No. 1055, Seq. ID No. 1075, Seq. ID No. 1077, Seq. ID No. 1105, Seq. ID No. 1107, Seq. ID No. 1131, Seq. ID No. 1135, Seq. ID No. 1137, Seq. ID No. 1139, Seq. ID No. 1151, Seq. ID No. 1165, Seq. ID No. 1167, Seq. ID No. 1169, Seq. ID No. 1173, Seq. ID No. 1183, Seq. ID No. 1195, Seq. ID No. 1197, Seq. ID No. 1203, Seq. ID No. 1213, Seq. ID No. 1223, Seq. ID No. 1227, Seq. ID No. 1229, Seq. ID No. 1237, Seq. ID No. 1243, Seq. ID No. 1245, Seq. ID No. 1259, Seq. ID No. 1265, Seq. ID No. 1267, Seq. ID No. 1269, Seq. ID No. 1271, Seq. ID No. 1283, Seq. ID No. 1287, Seq. ID No. 1289, Seq. ID No. 1291, Seq. ID No. 1295, Seq. ID No. 1297, Seq. ID No. 1303, Seq. ID No. 1305, Seq. ID No. 1315, Seq. ID No. 1317, Seq. ID No. 1319, Seq. ID No. 1325, Seq. ID No. 1327, Seq. ID No. 1329, Seq. ID No. 1331, Seq. ID No. 1337, Seq. ID No. 1341, Seq. ID No. 1343, Seq. ID No. 1347, Seq. ID No. 1349, Seq. ID No. 1351, Seq. ID No. 1353, Seq. ID No. 1365, Seq. ID No. 1367, Seq. ID No. 1369, Seq. ID No. 1371, Seq. ID No. 1381, Seq. ID No. 1401, Seq. ID No. 1429, Seq. ID No. 1435, Seq. ID No. 1445, Seq. ID No. 1447, Seq. ID No. 1481, Seq. ID No. 1513, Seq. ID No. 1515, Seq. ID No. 1517, Seq. ID No. 1529, Seq. ID No. 1533, Seq. ID No. 1535, Seq. ID No. 1545, Seq. ID No. 1547, Seq. ID No. 1551, Seq. ID No. 1553, Seq. ID No. 1559, Seq. ID No. 1561, Seq. ID No. 1563, Seq. ID No. 1571, Seq. ID No. 1579, Seq. ID No. 1583, Seq. ID No. 1585, Seq. ID No. 1621, Seq. ID No. 1633, Seq. ID No. 1639, Seq. ID No. 1643, Seq. ID No. 1645, Seq. ID No. 1647, Seq. ID No. 1649, Seq. ID No. 1653, Seq. ID No. 1661, Seq. ID No. 1663, Seq. ID No. 1665, Seq. ID No. 1671, Seq. ID No. 1673, Seq. ID No. 1677, Seq. ID No. 1679, Seq. ID No. 1681, Seq. ID No. 1693, Seq. ID No. 1707, Seq. ID No. 1711, Seq. ID No. 1731, Seq. ID No. 1733, Seq. ID No. 1737, Seq. ID No. 1753, Seq. ID No. 1755, Seq. ID No. 1757, Seq. ID No. 1765, Seq. ID No. 1767, Seq. ID No. 1771, Seq. ID No. 1781, Seq. ID No. 1787, Seq. ID No. 1791, Seq. ID No. 1797, Seq. ID No. 1805, Seq. ID No. 1819, Seq. ID No. 1821, Seq. ID No. 1823, Seq. ID No. 1839, Seq. ID No. 1857, Seq. ID No. 1869, Seq. ID No. 1875, Seq. ID No. 1891, Seq. ID No. 1893, Seq. ID No. 1895, Seq. ID No. 1915, Seq. ID No. 1917, Seq. ID No. 1919, Seq. ID No. 1923, Seq. ID No. 1927, Seq. ID No. 1929, Seq. ID No. 1969, Seq. ID No. 1981, Seq. ID No. 1991, Seq. ID No. 1993, Seq. ID No. 2005, Seq. ID No. 2013, Seq. ID No. 2017, Seq. ID No. 2019, Seq. ID No. 2025, Seq. ID No. 2027, Seq. ID No. 2033, Seq. ID No. 2035, Seq. ID No. 2041, Seq. ID No. 2049, Seq. ID No. 2053, Seq. ID No. 2055, Seq. ID No. 2087, Seq. ID No. 2089, Seq. ID No. 2091, Seq. ID No. 2105, Seq. ID No. 2107, Seq. ID No. 2109, Seq. ID No. 2113, Seq. ID No. 2115, Seq. ID No. 2121, Seq. ID No. 2123, Seq. ID No. 2125, Seq. ID No. 2127, Seq. ID No. 2149, Seq. ID No. 2151, Seq. ID No. 2155, Seq. ID No. 2157, Seq. ID No. 2159, Seq. ID No. 2171, Seq. ID No. 2173, Seq. ID No. 2177, Seq. ID No. 2187, Seq. ID No. 2191, Seq. ID No. 2205, Seq. ID No. 2237, Seq. ID No. 2239, Seq. ID No. 2245, Seq. ID No. 2249, Seq. ID No. 2251, Seq. ID No. 2255, Seq. ID No. 2259, Seq. ID No. 2271, Seq. ID No. 2283, Seq. ID No. 2289, Seq. ID No. 2291, Seq. ID No. 2293, Seq. ID No. 2295, Seq. ID No. 2303, Seq. ID No. 2305, Seq. ID No. 2307, Seq. ID No. 2317, Seq. ID No. 2319, Seq. ID No. 2321, Seq. ID No. 2323, Seq. ID No. 2325, Seq. ID No. 2327, Seq. ID No. 2329, Seq. ID No. 2333, Seq. ID No. 2343, Seq. ID No. 2347, Seq. ID No. 2349, Seq. ID No. 2351, Seq. ID No. 2353, Seq. ID No. 2355, Seq. ID No. 2357, Seq. ID No. 2365, Seq. ID No. 2367, Seq. ID No. 2371, Seq. ID No. 2375, Seq. ID No. 2387, Seq. ID No. 2389, Seq. ID No. 2391, Seq. ID No. 2393, Seq. ID No. 2407, Seq. ID No. 2419, Seq. ID No. 2421, Seq. ID No. 2425, Seq. ID No. 2427, Seq. ID No. 2429, Seq. ID No. 2433, Seq. ID No. 2449, Seq. ID No. 2459, Seq. ID No. 2487, Seq. ID No. 2489, Seq. ID No. 2491, Seq. ID No. 2493, Seq. ID No. 2497, Seq. ID No. 2499, Seq. ID No. 2501, Seq. ID No. 2503, Seq. ID No. 2507, Seq. ID No. 2515, Seq. ID No. 2517, Seq. ID No. 2523, Seq. ID No. 2525, Seq. ID No. 2527, Seq. ID No. 2531, Seq. ID No. 2533, Seq. ID No. 2535, Seq. ID No. 2537, Seq. ID No. 2541, Seq. ID No. 2543, Seq. ID No. 2545, Seq. ID No. 2555, Seq. ID No. 2557, Seq. ID No. 2565, Seq. ID No. 2569, Seq. ID No. 2571, Seq. ID No. 2583, Seq. ID No. 2591, Seq. ID No. 2597, Seq. ID No. 2599, Seq. ID No. 2601, Seq. ID No. 2607, Seq. ID No. 2611, Seq. ID No. 2621, Seq. ID No. 2627, Seq. ID No. 2629, Seq. ID No. 2651, Seq. ID No. 2679, Seq. ID No. 2681, Seq. ID No. 2683, Seq. ID No. 2691, Seq. ID No. 2693, Seq. ID No. 2695, Seq. ID No. 2697, Seq. ID No. 2699, Seq. ID No. 2701, Seq. ID No. 2703, Seq. ID No. 2705, Seq. ID No. 2707, Seq. ID No. 2717, Seq. ID No. 2729, Seq. ID No. 2731, Seq. ID No. 2733, Seq. ID No. 2783, Seq. ID No. 2785, Seq. ID No. 2801, Seq. ID No. 2809, Seq. ID No. 2813, Seq. ID No. 2815, Seq. ID No. 2819, Seq. ID No. 2821, Seq. ID No. 2823, Seq. ID No. 2831, Seq. ID No. 2833, Seq. ID No. 2835, Seq. ID No. 2837, Seq. ID No. 2839, Seq. ID No. 2841, Seq. ID No. 2845, Seq. ID No. 2853, Seq. ID No. 2855, Seq. ID No. 2859, Seq. ID No. 2861, Seq. ID No. 2873, Seq. ID No. 2881, Seq. ID No. 2893, Seq. ID No. 2905, Seq. ID No. 2935, Seq. ID No. 2937, Seq. ID No. 2939, Seq. ID No. 2941, Seq. ID No. 2947, Seq. ID No. 2949, Seq. ID No. 2951, Seq. ID No. 2969, Seq. ID No. 2973, Seq. ID No. 2975, Seq. ID No. 2977, Seq. ID No. 2981, Seq. ID No. 2983, Seq. ID No. 2987, Seq. ID No. 2991, Seq. ID No. 2995, Seq. ID No. 2997, Seq. ID No. 3005, Seq. ID No. 3007, Seq. ID No. 3009, Seq. ID No. 3011, Seq. ID No. 3013, Seq. ID No. 3023, Seq. ID No. 3025, Seq. ID No. 3027, Seq. ID No. 3029, Seq. ID No. 3031, Seq. ID No. 3043, Seq. ID No. 3055, Seq. ID No. 3063, Seq. ID No. 3071, Seq. ID No. 3073, Seq. ID No. 3075, Seq. ID No. 3077, Seq. ID No. 3119, Seq. ID No. 3121, Seq. ID No. 3123, Seq. ID No. 3145, Seq. ID No. 3155, Seq. ID No. 3157, Seq. ID No. 3163, Seq. ID No. 3165, Seq. ID No. 3173, Seq. ID No. 3183, Seq. ID No. 3193, Seq. ID No. 3201, Seq. ID No. 3213, Seq. ID No. 3215, Seq. ID No. 3217, Seq. ID No. 3219, Seq. ID No. 3241, Seq. ID No. 3243, Seq. ID No. 3245, Seq. ID No. 3249, Seq. ID No. 3255, Seq. ID No. 3257, Seq. ID No. 3261, Seq. ID No. 3263, Seq. ID No. 3269, Seq. ID No. 3275, Seq. ID No. 3277, Seq. ID No. 3297, Seq. ID No. 3317, Seq. ID No. 3323, Seq. ID No. 3325, Seq. ID No. 3331, Seq. ID No. 3335, Seq. ID No. 3337, Seq. ID No. 3339, Seq. ID No. 3349, Seq. ID No. 3351, Seq. ID No. 3353, Seq. ID No. 3355, Seq. ID No. 3357, Seq. ID No. 3359, Seq. ID No. 3375, Seq. ID No. 3377, Seq. ID No. 3389, Seq. ID No. 3391, Seq. ID No. 3395, Seq. ID No. 3405, Seq. ID No. 3415, Seq. ID No. 3421, Seq. ID No. 3425, Seq. ID No. 3427, Seq. ID No. 3433, Seq. ID No. 3441, Seq. ID No. 3449, Seq. ID No. 3451, Seq. ID No. 3453, Seq. ID No. 3455, Seq. ID No. 3457, Seq. ID No. 3463, Seq. ID No. 3481, Seq. ID No. 3491, Seq. ID No. 3493, Seq. ID No. 3503, Seq. ID No. 3505, Seq. ID No. 3517, Seq. ID No. 3519, Seq. ID No. 3527, Seq. ID No. 3531, Seq. ID No. 3539, Seq. ID No. 3561, Seq. ID No. 3573, Seq. ID No. 3581, Seq. ID No. 3587, Seq. ID No. 3593, Seq. ID No. 3595, Seq. ID No. 3611, Seq. ID No. 3617, Seq. ID No. 3619, Seq. ID No. 3621, Seq. ID No. 3625, Seq. ID No. 3629, Seq. ID No. 3635, Seq. ID No. 3677, Seq. ID No. 3679, Seq. ID No. 3689, Seq. ID No. 3717, Seq. ID No. 3723, Seq. ID No. 3727, Seq. ID No. 3741, Seq. ID No. 3745, Seq. ID No. 3753, Seq. ID No. 3755, Seq. ID No. 3757, Seq. ID No. 3761, Seq. ID No. 3763, Seq. ID No. 3765, Seq. ID No. 3781, Seq. ID No. 3783, Seq. ID No. 3793, Seq. ID No. 3799, Seq. ID No. 3807, Seq. ID No. 3813, Seq. ID No. 3815, Seq. ID No. 3817, Seq. ID No. 3837, Seq. ID No. 3839, Seq. ID No. 3849, Seq. ID No. 3851, Seq. ID No. 3853, Seq. ID No. 3859, Seq. ID No. 3861, Seq. ID No. 3863, Seq. ID No. 3865, Seq. ID No. 3879, Seq. ID No. 3889, Seq. ID No. 3891, Seq. ID No. 3895, Seq. ID No. 3903, Seq. ID No. 3915, Seq. ID No. 3923, Seq. ID No. 3925, Seq. ID No. 3959, Seq. ID No. 3963, Seq. ID No. 3973, Seq. ID No. 3975, Seq. ID No. 3993, Seq. ID No. 3995, Seq. ID No. 3997, Seq. ID No. 4005, Seq. ID No. 4007, Seq. ID No. 4009, Seq. ID No. 4025, Seq. ID No. 4027, Seq. ID No. 4039, Seq. ID No. 4041, Seq. ID No. 4043, Seq. ID No. 4045, Seq. ID No. 4047, Seq. ID No. 4049, Seq. ID No. 4061, Seq. ID No. 4071, Seq. ID No. 4073, Seq. ID No. 4079, Seq. ID No. 4081, Seq. ID No. 4083, Seq. ID No. 4101, Seq. ID No. 4103, Seq. ID No. 4111, Seq. ID No. 4133, Seq. ID No. 4135, Seq. ID No. 4137, Seq. ID No. 4139, Seq. ID No. 4141, Seq. ID No. 4143, Seq. ID No. 4145, Seq. ID No. 4151, Seq. ID No. 4153, Seq. ID No. 4181, Seq. ID No. 4221, Seq. ID No. 4223, Seq. ID No. 4225, Seq. ID No. 4227, Seq. ID No. 4231, Seq. ID No. 4233, Seq. ID No. 4241, Seq. ID No. 4247, Seq. ID No. 4253, Seq. ID No. 4257, Seq. ID No. 4259, Seq. ID No. 4261, Seq. ID No. 4263, Seq. ID No. 4265, Seq. ID No. 4269, Seq. ID No. 4275, Seq. ID No. 4285, Seq. ID No. 4287, Seq. ID No. 4297, Seq. ID No. 4307, Seq. ID No. 4311, Seq. ID No. 4313, Seq. ID No. 4343, Seq. ID No. 4379, Seq. ID No. 4385, Seq. ID No. 4389, Seq. ID No. 4391, Seq. ID No. 4399, Seq. ID No. 4403, Seq. ID No. 4437, Seq. ID No. 4439, Seq. ID No. 4441, Seq. ID No. 4469, Seq. ID No. 4473, Seq. ID No. 4481, Seq. ID No. 4483, Seq. ID No. 4485, Seq. ID No. 4493, Seq. ID No. 4495, Seq. ID No. 4497, Seq. ID No. 4513, Seq. ID No. 4515, Seq. ID No. 4527, Seq. ID No. 4529, Seq. ID No. 4533, Seq. ID No. 4587, Seq. ID No. 4589, Seq. ID No. 4639, Seq. ID No. 4641, Seq. ID No. 4643, Seq. ID No. 4659, Seq. ID No. 4681, Seq. ID No. 4687, Seq. ID No. 4695, Seq. ID No. 4705, Seq. ID No. 4707, Seq. ID No. 4709, Seq. ID No. 4711, Seq. ID No. 4715, Seq. ID No. 4717, Seq. ID No. 4737, Seq. ID No. 4759, Seq. ID No. 4813, Seq. ID No. 4827, Seq. ID No. 4853, Seq. ID No. 4863, Seq. ID No. 4869, Seq. ID No. 4871, Seq. ID No. 4873, Seq. ID No. 4875, Seq. ID No. 4919, Seq. ID No. 4921, Seq. ID No. 4923, Seq. ID No. 4929, Seq. ID No. 4931, Seq. ID No. 4949, Seq. ID No. 4951, Seq. ID No. 4975, Seq. ID No. 4987, Seq. ID No. 4999, Seq. ID No. 5013, Seq. ID No. 5031, Seq. ID No. 5061, Seq. ID No. 5063, Seq. ID No. 5069, Seq. ID No. 5085, Seq. ID No. 5103, Seq. ID No. 5115, Seq. ID No. 5123, Seq. ID No. 5125, Seq. ID No. 5129, Seq. ID No. 5131, Seq. ID No. 5133, Seq. ID No. 5135, Seq. ID No. 5137, Seq. ID No. 5139, Seq. ID No. 5141, Seq. ID No. 5159, Seq. ID No. 5165, Seq. ID No. 5175, Seq. ID No. 5197, Seq. ID No. 5199, Seq. ID No. 5205, Seq. ID No. 5207, Seq. ID No. 5213, Seq. ID No. 5219, Seq. ID No. 5223, Seq. ID No. 5267, Seq. ID No. 5273, Seq. ID No. 5275, Seq. ID No. 5277, Seq. ID No. 5279, Seq. ID No. 5281, Seq. ID No. 5285, Seq. ID No. 5327, Seq. ID No. 5329, Seq. ID No. 5331, Seq. ID No. 5341, Seq. ID No. 5377, Seq. ID No. 5379, Seq. ID No. 5385, Seq. ID No. 5387, Seq. ID No. 5405, Seq. ID No. 5413, Seq. ID No. 5415, Seq. ID No. 5433, Seq. ID No. 5453, Seq. ID No. 5459, Seq. ID No. 5461, Seq. ID No. 5463, Seq. ID No. 5465, Seq. ID No. 5491, Seq. ID No. 5497, Seq. ID No. 5509, Seq. ID No. 5513, Seq. ID No. 5515, Seq. ID No. 5517, Seq. ID No. 5525, Seq. ID No. 5529, Seq. ID No. 5531, Seq. ID No. 5533, Seq. ID No. 5543, Seq. ID No. 5545, Seq. ID No. 5569, Seq. ID No. 5587, Seq. ID No. 5589, Seq. ID No. 5591, Seq. ID No. 5593, Seq. ID No. 5595, Seq. ID No. 5597, Seq. ID No. 5609, Seq. ID No. 5611, Seq. ID No. 5621, Seq. ID No. 5623, Seq. ID No. 5625, Seq. ID No. 5633, Seq. ID No. 5637, Seq. ID No. 5639, Seq. ID No. 5641, Seq. ID No. 5643, Seq. ID No. 5649, Seq. ID No. 5659, Seq. ID No. 5667, Seq. ID No. 5681, Seq. ID No. 5683, Seq. ID No. 5685, Seq. ID No. 5687, Seq. ID No. 5689, Seq. ID No. 5699, Seq. ID No. 5703, Seq. ID No. 5705, Seq. ID No. 5709, Seq. ID No. 5711, Seq. ID No. 5719, Seq. ID No. 5729, Seq. ID No. 5733, Seq. ID No. 5735, Seq. ID No. 5739, Seq. ID No. 5763, Seq. ID No. 5765, Seq. ID No. 5767, Seq. ID No. 5771, Seq. ID No. 5783, Seq. ID No. 5785, Seq. ID No. 5791, Seq. ID No. 5797, Seq. ID No. 5829, Seq. ID No. 5879, Seq. ID No. 5883, Seq. ID No. 5885, Seq. ID No. 5889, Seq. ID No. 5893, Seq. ID No. 5895, Seq. ID No. 5899, Seq. ID No. 5901, Seq. ID No. 5905, Seq. ID No. 5909, Seq. ID No. 5921, Seq. ID No. 5923, Seq. ID No. 5927, Seq. ID No. 5929, Seq. ID No. 5939, Seq. ID No. 6023, Seq. ID No. 6025, Seq. ID No. 6027, Seq. ID No. 6043, Seq. ID No. 6047, Seq. ID No. 6053, Seq. ID No. 6059, Seq. ID No. 6063, Seq. ID No. 6067, Seq. ID No. 6087, Seq. ID No. 6089, Seq. ID No. 6091, Seq. ID No. 6095, Seq. ID No. 6103, Seq. ID No. 6107, Seq. ID No. 6109, Seq. ID No. 6111, Seq. ID No. 6115, Seq. ID No. 6117, Seq. ID No. 6135, Seq. ID No. 6137, Seq. ID No. 6159, Seq. ID No. 6161, Seq. ID No. 6163, Seq. ID No. 6165, Seq. ID No. 6169, Seq. ID No. 6183, Seq. ID No. 6185, Seq. ID No. 6187, Seq. ID No. 6189, Seq. ID No. 6191, Seq. ID No. 6197, Seq. ID No. 6203, Seq. ID No. 6205, Seq. ID No. 6209, Seq. ID No. 6217, Seq. ID No. 6221, Seq. ID No. 6223, Seq. ID No. 6225, Seq. ID No. 6227, Seq. ID No. 6229, Seq. ID No. 6235, Seq. ID No. 6237, Seq. ID No. 6241, Seq. ID No. 6243, Seq. ID No. 6245, Seq. ID No. 6249, Seq. ID No. 6253, Seq. ID No. 6259, Seq. ID No. 6263, Seq. ID No. 6265, Seq. ID No. 6267, Seq. ID No. 6273, Seq. ID No. 6275, Seq. ID No. 6283, Seq. ID No. 6285, Seq. ID No. 6289, Seq. ID No. 6301, Seq. ID No. 6303, Seq. ID No. 6325, Seq. ID No. 6327, Seq. ID No. 6363, Seq. ID No. 6365, Seq. ID No. 6377, Seq. ID No. 6381, Seq. ID No. 6383, Seq. ID No. 6415, Seq. ID No. 6425, Seq. ID No. 6427, Seq. ID No. 6429, Seq. ID No. 6431, Seq. ID No. 6473, Seq. ID No. 6479, Seq. ID No. 6497, Seq. ID No. 6517, Seq. ID No. 6541, Seq. ID No. 6543, Seq. ID No. 6555, Seq. ID No. 6557, Seq. ID No. 6571, Seq. ID No. 6573, Seq. ID No. 6581, Seq. ID No. 6593, Seq. ID No. 6595, Seq. ID No. 6597, Seq. ID No. 6599, Seq. ID No. 6601, Seq. ID No. 6603, Seq. ID No. 6631, Seq. ID No. 6637, Seq. ID No. 6639, Seq. ID No. 6643, Seq. ID No. 6645, and Seq. ID No. 6649.

15. The polynucleotide of claim 7, wherein the polynucleotide encodes a polypeptide, which is predicted by HMM Signal P algorithm analysis as being a non-secretory protein, and the polynucleotide is selected from one of Seq. ID No. 1, Seq. ID No. 5, Seq. ID No. 11, Seq. ID No. 13, Seq. ID No. 15, Seq. ID No. 17, Seq. ID No. 23, Seq. ID No. 25, Seq. ID No. 27, Seq. ID No. 29, Seq. ID No. 35, Seq. ID No. 37, Seq. ID No. 39, Seq. ID No. 41, Seq. ID No. 43, Seq. ID No. 51, Seq. ID No. 55, Seq. ID No. 57, Seq. ID No. 59, Seq. ID No. 61, Seq. ID No. 63, Seq. ID No. 69, Seq. ID No. 71, Seq. ID No. 73, Seq. ID No. 75, Seq. ID No. 77, Seq. ID No. 79, Seq. ID No. 81, Seq. ID No. 83, Seq. ID No. 89, Seq. ID No. 91, Seq. ID No. 93, Seq. ID No. 95, Seq. ID No. 97, Seq. ID No. 99, Seq. ID No. 101, Seq. ID No. 103, Seq. ID No. 107, Seq. ID No. 109, Seq. ID No. 113, Seq. ID No. 115, Seq. ID No. 117, Seq. ID No. 119, Seq. ID No. 121, Seq. ID No. 125, Seq. ID No. 127, Seq. ID No. 129, Seq. ID No. 135, Seq. ID No. 137, Seq. ID No. 139, Seq. ID No. 143, Seq. ID No. 145, Seq. ID No. 147, Seq. ID No. 149, Seq. ID No. 151, Seq. ID No. 153, Seq. ID No. 155, Seq. ID No. 157, Seq. ID No. 165, Seq. ID No. 167, Seq. ID No. 169, Seq. ID No. 173, Seq. ID No. 175, Seq. ID No. 177, Seq. ID No. 179, Seq. ID No. 181, Seq. ID No. 183, Seq. ID No. 185, Seq. ID No. 187, Seq. ID No. 189, Seq. ID No. 193, Seq. ID No. 195, Seq. ID No. 197, Seq. ID No. 199, Seq. ID No. 201, Seq. ID No. 203, Seq. ID No. 205, Seq. ID No. 207, Seq. ID No. 219, Seq. ID No. 221, Seq. ID No. 223, Seq. ID No. 227, Seq. ID No. 229, Seq. ID No. 231, Seq. ID No. 233, Seq. ID No. 239, Seq. ID No. 241, Seq. ID No. 243, Seq. ID No. 245, Seq. ID No. 247, Seq. ID No. 253, Seq. ID No. 261, Seq. ID No. 263, Seq. ID No. 265, Seq. ID No. 267, Seq. ID No. 269, Seq. ID No. 275, Seq. ID No. 279, Seq. ID No. 281, Seq. ID No. 283, Seq. ID No. 291, Seq. ID No. 293, Seq. ID No. 295, Seq. ID No. 297, Seq. ID No. 299, Seq. ID No. 301, Seq. ID No. 303, Seq. ID No. 305, Seq. ID No. 307, Seq. ID No. 309, Seq. ID No. 311, Seq. ID No. 313, Seq. ID No. 315, Seq. ID No. 317, Seq. ID No. 319, Seq. ID No. 321, Seq. ID No. 323, Seq. ID No. 325, Seq. ID No. 331, Seq. ID No. 333, Seq. ID No. 335, Seq. ID No. 337, Seq. ID No. 339, Seq. ID No. 341, Seq. ID No. 343, Seq. ID No. 345, Seq. ID No. 347, Seq. ID No. 349, Seq. ID No. 351, Seq. ID No. 353, Seq. ID No. 355, Seq. ID No. 361, Seq. ID No. 363, Seq. ID No. 365, Seq. ID No. 367, Seq. ID No. 369, Seq. ID No. 371, Seq. ID No. 377, Seq. ID No. 379, Seq. ID No. 381, Seq. ID No. 383, Seq. ID No. 385, Seq. ID No. 387, Seq. ID No. 389, Seq. ID No. 391, Seq. ID No. 395, Seq. ID No. 397, Seq. ID No. 399, Seq. ID No. 405, Seq. ID No. 407, Seq. ID No. 409, Seq. ID No. 411, Seq. ID No. 413, Seq. ID No. 415, Seq. ID No. 417, Seq. ID No. 419, Seq. ID No. 421, Seq. ID No. 423, Seq. ID No. 425, Seq. ID No. 427, Seq. ID No. 429, Seq. ID No. 431, Seq. ID No. 433, Seq. ID No. 435, Seq. ID No. 437, Seq. ID No. 439, Seq. ID No. 441, Seq. ID No. 443, Seq. ID No. 445, Seq. ID No. 447, Seq. ID No. 449, Seq. ID No. 451, Seq. ID No. 453, Seq. ID No. 459, Seq. ID No. 461, Seq. ID No. 463, Seq. ID No. 465, Seq. ID No. 467, Seq. ID No. 469, Seq. ID No. 471, Seq. ID No. 473, Seq. ID No. 475, Seq. ID No. 479, Seq. ID No. 481, Seq. ID No. 483, Seq. ID No. 485, Seq. ID No. 501, Seq. ID No. 507, Seq. ID No. 517, Seq. ID No. 519, Seq. ID No. 521, Seq. ID No. 523, Seq. ID No. 529, Seq. ID No. 531, Seq. ID No. 533, Seq. ID No. 535, Seq. ID No. 537, Seq. ID No. 539, Seq. ID No. 541, Seq. ID No. 543, Seq. ID No. 551, Seq. ID No. 553, Seq. ID No. 555, Seq. ID No. 559, Seq. ID No. 561, Seq. ID No. 563, Seq. ID No. 565, Seq. ID No. 571, Seq. ID No. 573, Seq. ID No. 581, Seq. ID No. 583, Seq. ID No. 585, Seq. ID No. 587, Seq. ID No. 589, Seq. ID No. 595, Seq. ID No. 597, Seq. ID No. 601, Seq. ID No. 603, Seq. ID No. 605, Seq. ID No. 607, Seq. ID No. 609, Seq. ID No. 611, Seq. ID No. 613, Seq. ID No. 615, Seq. ID No. 619, Seq. ID No. 621, Seq. ID No. 623, Seq. ID No. 625, Seq. ID No. 627, Seq. ID No. 629, Seq. ID No. 631, Seq. ID No. 633, Seq. ID No. 635, Seq. ID No. 649, Seq. ID No. 651, Seq. ID No. 663, Seq. ID No. 665, Seq. ID No. 671, Seq. ID No. 673, Seq. ID No. 681, Seq. ID No. 683, Seq. ID No. 685, Seq. ID No. 687, Seq. ID No. 689, Seq. ID No. 691, Seq. ID No. 693, Seq. ID No. 695, Seq. ID No. 697, Seq. ID No. 699, Seq. ID No. 701, Seq. ID No. 703, Seq. ID No. 705, Seq. ID No. 707, Seq. ID No. 709, Seq. ID No. 711, Seq. ID No. 713, Seq. ID No. 727, Seq. ID No. 729, Seq. ID No. 731, Seq. ID No. 733, Seq. ID No. 735, Seq. ID No. 737, Seq. ID No. 739, Seq. ID No. 741, Seq. ID No. 743, Seq. ID No. 753, Seq. ID No. 767, Seq. ID No. 771, Seq. ID No. 773, Seq. ID No. 775, Seq. ID No. 783, Seq. ID No. 785, Seq. ID No. 787, Seq. ID No. 789, Seq. ID No. 791, Seq. ID No. 793, Seq. ID No. 795, Seq. ID No. 797, Seq. ID No. 799, Seq. ID No. 801, Seq. ID No. 803, Seq. ID No. 805, Seq. ID No. 811, Seq. ID No. 813, Seq. ID No. 815, Seq. ID No. 817, Seq. ID No. 821, Seq. ID No. 823, Seq. ID No. 827, Seq. ID No. 829, Seq. ID No. 831, Seq. ID No. 833, Seq. ID No. 835, Seq. ID No. 837, Seq. ID No. 839, Seq. ID No. 841, Seq. ID No. 843, Seq. ID No. 845, Seq. ID No. 847, Seq. ID No. 849, Seq. ID No. 851, Seq. ID No. 853, Seq. ID No. 855, Seq. ID No. 857, Seq. ID No. 859, Seq. ID No. 861, Seq. ID No. 865, Seq. ID No. 867, Seq. ID No. 869, Seq. ID No. 871, Seq. ID No. 873, Seq. ID No. 875, Seq. ID No. 877, Seq. ID No. 879, Seq. ID No. 881, Seq. ID No. 883, Seq. ID No. 885, Seq. ID No. 887, Seq. ID No. 889, Seq. ID No. 891, Seq. ID No. 895, Seq. ID No. 897, Seq. ID No. 899, Seq. ID No. 903, Seq. ID No. 905, Seq. ID No. 907, Seq. ID No. 909, Seq. ID No. 911, Seq. ID No. 913,Seq. ID No. 915, Seq. ID No. 917, Seq. ID No. 919, Seq. ID No. 921, Seq. ID No. 923, Seq. ID No. 925, Seq. ID No. 927, Seq. ID No. 929, Seq. ID No. 931, Seq. ID No. 933, Seq. ID No. 939, Seq. ID No. 945, Seq. ID No. 947, Seq. ID No. 949, Seq. ID No. 951, Seq. ID No. 953, Seq. ID No. 959, Seq. ID No. 961, Seq. ID No. 963, Seq. ID No. 95, Seq. ID No. 969, Seq. ID No. 971, Seq. ID No. 973, Seq. ID No. 975, Seq. ID No. 977, Seq. ID No. 987, Seq. ID No. 989, Seq. ID No. 991, Seq. ID No. 993, Seq. ID No. 995, Seq. ID No. 997, Seq. ID No. 999, Seq. ID No. 1017, Seq. ID No. 1021, Seq. ID No. 1023, Seq. ID No. 1025, Seq. ID No. 1027, Seq. ID No. 1041, Seq. ID No. 1043, Seq. ID No. 1047, Seq. ID No. 1049, Seq. ID No. 1051, Seq. ID No. 1053, Seq. ID No. 1057, Seq. ID No. 1059, Seq. ID No. 1061, Seq. ID No. 1063, Seq. ID No. 1065, Seq. ID No. 1067, Seq. ID No. 1069, Seq. ID No. 1071, Seq. ID No. 1073, Seq. ID No. 1079, Seq. ID No. 1081, Seq. ID No. 1083, Seq. ID No. 1085, Seq. ID No. 1087, Seq. ID No. 1089, Seq. ID No. 1091, Seq. ID No. 1093, Seq. ID No. 1095, Seq. ID No. 1097, Seq. ID No. 1099, Seq. ID No. 1101, Seq. ID No. 1103, Seq. ID No. 1109, Seq. ID No. 1111, Seq. ID No. 1113, Seq. ID No. 1115, Seq. ID No. 1117, Seq. ID No. 1119, Seq. ID No. 1121, Seq. ID No. 1123, Seq. ID No. 1125, Seq. ID No. 1127, Seq. ID No. 1129, Seq. ID No. 1133, Seq. ID No. 1141, Seq. ID No. 1143, Seq. ID No. 1145, Seq. ID No. 1147, Seq. ID No. 1149, Seq. ID No. 1153, Seq. ID No. 1155, Seq. ID No. 1157, Seq. ID No. 1159, Seq. ID No. 1161, Seq. ID No. 1163, Seq. ID No. 1171, Seq. ID No. 1175, Seq. ID No. 1177, Seq. ID No. 1179, Seq. ID No. 1181, Seq. ID No. 1185, Seq. ID No. 1187, Seq. ID No. 1189, Seq. ID No. 1191, Seq. ID No. 1193, Seq. ID No. 1199, Seq. ID No. 1201, Seq. ID No. 1205, Seq. ID No. 1207, Seq. ID No. 1209, Seq. ID No. 1211, Seq. ID No. 1215, Seq. ID No. 1217, Seq. ID No. 1219, Seq. ID No. 1221, Seq. ID No. 1225, Seq. ID No. 1231, Seq. ID No. 1233, Seq. ID No. 1235, Seq. ID No. 1239, Seq. ID No. 1241, Seq. ID No. 1247, Seq. ID No. 1249, Seq. ID No. 1251, Seq. ID No. 1253, Seq. ID No. 1255, Seq. ID No. 1257, Seq. ID No. 1261, Seq. ID No. 1263, Seq. ID No. 1273, Seq. ID No. 1275, Seq. ID No. 1277, Seq. ID No. 1279, Seq. ID No. 1281, Seq. ID No. 1285, Seq. ID No. 1293, Seq. ID No. 1299, Seq. ID No. 1301, Seq. ID No. 1307, Seq. ID No. 1309, Seq. ID No. 1311, Seq. ID No. 1313, Seq. ID No. 1321, Seq. ID No. 1323, Seq. ID No. 1333, Seq. ID No. 1335, Seq. ID No. 1339, Seq. ID No. 1345, Seq. ID No. 1355, Seq. ID No. 1357, Seq. ID No. 1359, Seq. ID No. 1361, Seq. ID No. 1363, Seq. ID No. 1373, Seq. ID No. 1375, Seq. ID No. 1377, Seq. ID No. 1379, Seq. ID No. 1383, Seq. ID No. 1385, Seq. ID No. 1387, Seq. ID No. 1389, Seq. ID No. 1391, Seq. ID No. 1393, Seq. ID No. 1395, Seq. ID No. 1397, Seq. ID No. 1399, Seq. ID No. 1403, Seq. ID No. 1405, Seq. ID No. 1407, Seq. ID No. 1409, Seq. ID No. 1411, Seq. ID No. 1413, Seq. ID No. 1415, Seq. ID No. 1417, Seq. ID No. 1419, Seq. ID No. 1421, Seq. ID No. 1423, Seq. ID No. 1425, Seq. ID No. 1427, Seq. ID No. 1431, Seq. ID No. 1433, Seq. ID No. 1437, Seq. ID No. 1439, Seq. ID No. 1441, Seq. ID No. 1443, Seq. ID No. 1449, Seq. ID No. 1451, Seq. ID No. 1453, Seq. ID No. 1455, Seq. ID No. 1457, Seq. ID No. 1459, Seq. ID No. 1461, Seq. ID No. 1463, Seq. ID No. 1465, Seq. ID No. 1467, Seq. ID No. 1469, Seq. ID No. 1471, Seq. ID No. 1473, Seq. ID No. 1475, Seq. ID No. 1477, Seq. ID No. 1479, Seq. ID No. 1483, Seq. ID No. 1485, Seq. ID No. 1487, Seq. ID No. 1489, Seq. ID No. 1491, Seq. ID No. 1493, Seq. ID No. 1495, Seq. ID No. 1497, Seq. ID No. 1499, Seq. ID No. 1501, Seq. ID No. 1503, Seq. ID No. 1505, Seq. ID No. 1507, Seq. ID No. 1509, Seq. ID No. 1511, Seq. ID No. 1519, Seq. ID No. 1521, Seq. ID No. 1523, Seq. ID No. 1525, Seq. ID No. 1527, Seq. ID No. 1531, Seq. ID No. 1537, Seq. ID No. 1539, Seq. ID No. 1541, Seq. ID No. 1543, Seq. ID No. 1549, Seq. ID No. 1555, Seq. ID No. 1557, Seq. ID No. 1565, Seq. ID No. 1567, Seq. ID No. 1569, Seq. ID No. 1573, Seq. ID No. 1575, Seq. ID No. 1577, Seq. ID No. 1581, Seq. ID No. 1587, Seq. ID No. 1589, Seq. ID No. 1591, Seq. ID No. 1593, Seq. ID No. 1595, Seq. ID No. 1597, Seq. ID No. 1601, Seq. ID No. 1603, Seq. ID No. 1605, Seq. ID No. 1607, Seq. ID No. 1609, Seq. ID No. 1611, Seq. ID No. 1613, Seq. ID No. 1615, Seq. ID No. 1617, Seq. ID No. 1619, Seq. ID No. 1623, Seq. ID No. 1625, Seq. ID No. 1627, Seq. ID No. 1629, Seq. ID No. 1631, Seq. ID No. 1635, Seq. ID No. 1637, Seq. ID No. 1641, Seq. ID No. 1651, Seq. ID No. 1655, Seq. ID No. 1657, Seq. ID No. 1659, Seq. ID No. 1667, Seq. ID No. 1669, Seq. ID No. 1675, Seq. ID No. 1683, Seq. ID No. 1685, Seq. ID No. 1687, Seq. ID No. 1689, Seq. ID No. 1691, Seq. ID No. 1695, Seq. ID No. 1697, Seq. ID No. 1699, Seq. ID No. 1701, Seq. ID No. 1703, Seq. ID No. 1705, Seq. ID No. 1709, Seq. ID No. 1713, Seq. ID No. 1715, Seq. ID No. 1717, Seq. ID No. 1719, Seq. ID No. 1721, Seq. ID No. 1723, Seq. ID No. 1725, Seq. ID No. 1727, Seq. ID No. 1729, Seq. ID No. 1735, Seq. ID No. 1739, Seq. ID No. 1741, Seq. ID No. 1743, Seq. ID No. 1745, Seq. ID No. 1747, Seq. ID No. 1749, Seq. ID No. 1751, Seq. ID No. 1759, Seq. ID No. 1761, Seq. ID No. 1763, Seq. ID No. 1769, Seq. ID No. 1773, Seq. ID No. 1775, Seq. ID No. 1777, Seq. ID No. 1779, Seq. ID No. 1783, Seq. ID No. 1785, Seq. ID No. 1789, Seq. ID No. 1793, Seq. ID No. 1795, Seq. ID No. 1799, Seq. ID No. 1801, Seq. ID No. 1803, Seq. ID No. 1807, Seq. ID No. 1809, Seq. ID No. 1811, Seq. ID No. 1813, Seq. ID No. 1815, Seq. ID No. 1817, Seq. ID No. 1825, Seq. ID No. 1827, Seq. ID No. 1829, Seq. ID No. 1831, Seq. ID No. 1833, Seq. ID No. 1835, Seq. ID No. 1837, Seq. ID No. 1841, Seq. ID No. 1843, Seq. ID No. 1845, Seq. ID No. 1847, Seq. ID No. 1849, Seq. ID No. 1851, Seq. ID No. 1853, Seq. ID No. 1855, Seq. ID No. 1859, Seq. ID No. 1861, Seq. ID No. 1863, Seq. ID No. 1865, Seq. ID No. 1867, Seq. ID No. 1871, Seq. ID No. 1873, Seq. ID No. 1877, Seq. ID No. 1879, Seq. ID No. 1881, Seq. ID No. 1883, Seq. ID No. 1885, Seq. ID No. 1887, Seq. ID No. 1889, Seq. ID No. 1897, Seq. ID No. 1899, Seq. ID No. 1901, Seq. ID No. 1903, Seq. ID No. 1905, Seq. ID No. 1907, Seq. ID No. 1909, Seq. ID No. 1911, Seq. ID No. 1913, Seq. ID No. 1921, Seq. ID No. 1925, Seq. ID No. 1931, Seq. ID No. 1933, Seq. ID No. 1935, Seq. ID No. 1937, Seq. ID No. 1939, Seq. ID No. 1941, Seq. ID No. 1943, Seq. ID No. 1945, Seq. ID No. 1947, Seq. ID No. 1949, Seq. ID No. 1951, Seq. ID No. 1953, Seq. ID No. 1955, Seq. ID No. 1957, Seq. ID No. 1959, Seq. ID No. 1961, Seq. ID No. 1963, Seq. ID No. 1965, Seq. ID No. 1967, Seq. ID No. 1971, Seq. ID No. 1973, Seq. ID No. 1975, Seq. ID No. 1977, Seq. ID No. 1979, Seq. ID No. 1983, Seq. ID No. 1985, Seq. ID No. 1987, Seq. ID No. 1989, Seq. ID No. 1995, Seq. ID No. 1997, Seq. ID No. 1999, Seq. ID No. 2001, Seq. ID No. 2003, Seq. ID No. 2007, Seq. ID No. 2009, Seq. ID No. 2011, Seq. ID No. 2015, Seq. ID No. 2021, Seq. ID No. 2023, Seq. ID No. 2029, Seq. ID No. 2031, Seq. ID No. 2037, Seq. ID No. 2039, Seq. ID No. 2043, Seq. ID No. 2045, Seq. ID No. 2047, Seq. ID No. 2051, Seq. ID No. 2057, Seq. ID No. 2059, Seq. ID No. 2061, Seq. ID No. 2063, Seq. ID No. 2065, Seq. ID No. 2067, Seq. ID No. 2069, Seq. ID No. 2071, Seq. ID No. 2073, Seq. ID No. 2075, Seq. ID No. 2077, Seq. ID No. 2079, Seq. ID No. 2081, Seq. ID No. 2083, Seq. ID No. 2085, Seq. ID No. 2093, Seq. ID No. 2095, Seq. ID No. 2097, Seq. ID No. 2099, Seq. ID No. 2101, Seq. ID No. 2103, Seq. ID No. 2111, Seq. ID No. 2117, Seq. ID No. 2119, Seq. ID No. 2129, Seq. ID No. 2131, Seq. ID No. 2133, Seq. ID No. 2135, Seq. ID No. 2137, Seq. ID No. 2139, Seq. ID No. 2141, Seq. ID No. 2143, Seq. ID No. 2145, Seq. ID No. 2147, Seq. ID No. 2153, Seq. ID No. 2161, Seq. ID No. 2163, Seq. ID No. 2165, Seq. ID No. 2167, Seq. ID No. 2169, Seq. ID No. 2175, Seq. ID No. 2179, Seq. ID No. 2181, Seq. ID No. 2183, Seq. ID No. 2185, Seq. ID No. 2189, Seq. ID No. 2193, Seq. ID No. 2195, Seq. ID No. 2197, Seq. ID No. 2199, Seq. ID No. 2201, Seq. ID No. 2203, Seq. ID No. 2207, Seq. ID No. 2209, Seq. ID No. 2211, Seq. ID No. 2213, Seq. ID No. 2215, Seq. ID No. 2217, Seq. ID No. 2219, Seq. ID No. 2221, Seq. ID No. 2223, Seq. ID No. 2225, Seq. ID No. 2227, Seq. ID No. 2229, Seq. ID No. 2231, Seq. ID No. 2233, Seq. ID No. 2235, Seq. ID No. 2241, Seq. ID No. 2243, Seq. ID No. 2247, Seq. ID No. 2253, Seq. ID No. 2257, Seq. ID No. 2261, Seq. ID No. 2263, Seq. ID No. 2265, Seq. ID No. 2267, Seq. ID No. 2269, Seq. ID No. 2273, Seq. ID No. 2275, Seq. ID No. 2277, Seq. ID No. 2279, Seq. ID No. 2281, Seq. ID No. 2285, Seq. ID No. 2287, Seq. ID No. 2297, Seq. ID No. 2299, Seq. ID No. 2301, Seq. ID No. 2309, Seq. ID No. 2311, Seq. ID No. 2313, Seq. ID No. 2315, Seq. ID No. 2331, Seq. ID No. 2335, Seq. ID No. 2337, Seq. ID No. 2339, Seq. ID No. 2341, Seq. ID No. 2345, Seq. ID No. 2359, Seq. ID No. 2361, Seq. ID No. 2363, Seq. ID No. 2369, Seq. ID No. 2373, Seq. ID No. 2377, Seq. ID No. 2379, Seq. ID No. 2381, Seq. ID No. 2383, Seq. ID No. 2385, Seq. ID No. 2395, Seq. ID No. 2397, Seq. ID No. 2399, Seq. ID No. 2401, Seq. ID No. 2403, Seq. ID No. 2405, Seq. ID No. 2409, Seq. ID No. 2411, Seq. ID No. 2413, Seq. ID No. 2415, Seq. ID No. 2417, Seq. ID No. 2423, Seq. ID No. 2431, Seq. ID No. 2435, Seq. ID No. 2437, Seq. ID No. 2439, Seq. ID No. 2441, Seq. ID No. 2443, Seq. ID No. 2445, Seq. ID No. 2447, Seq. ID No. 2451, Seq. ID No. 2453, Seq. ID No. 2455, Seq. ID No. 2457, Seq. ID No. 2461, Seq. ID No. 2463, Seq. ID No. 2465, Seq. ID No. 2467, Seq. ID No. 2469, Seq. ID No. 2471, Seq. ID No. 2473, Seq. ID No. 2475, Seq. ID No. 2477, Seq. ID No. 2479, Seq. ID No. 2481, Seq. ID No. 2483, Seq. ID No. 2485, Seq. ID No. 2495, Seq. ID No. 2505, Seq. ID No. 2509, Seq. ID No. 2511, Seq. ID No. 2513, Seq. ID No. 2519, Seq. ID No. 2521, Seq. ID No. 2529, Seq. ID No. 2539, Seq. ID No. 2547, Seq. ID No. 2549, Seq. ID No. 2551, Seq. ID No. 2553, Seq. ID No. 2559, Seq. ID No. 2561, Seq. ID No. 2563, Seq. ID No. 2567, Seq. ID No. 2573, Seq. ID No. 2575, Seq. ID No. 2577, Seq. ID No. 2579, Seq. ID No. 2581, Seq. ID No. 2585, Seq. ID No. 2587, Seq. ID No. 2589, Seq. ID No. 2593, Seq. ID No. 2595, Seq. ID No. 2603, Seq. ID No. 2605, Seq. ID No. 2609, Seq. ID No. 2613, Seq. ID No. 2615, Seq. ID No. 2617, Seq. ID No. 2619, Seq. ID No. 2623, Seq. ID No. 2625, Seq. ID No. 2631, Seq. ID No. 2633, Seq. ID No, 2635, Seq. ID No. 2637, Seq. ID No. 2639, Seq. ID No. 2641, Seq. ID No. 2643, Seq. ID No. 2645, Seq. ID No. 2647, Seq. ID No. 2649, Seq. ID No. 2653, Seq. ID No. 2655, Seq. ID No. 2657, Seq. ID No. 2659, Seq. ID No. 2661, Seq. ID No. 2663, Seq. ID No. 2665, Seq. ID No. 2667, Seq. ID No. 2669, Seq. ID No. 2671, Seq. ID No. 2673, Seq. ID No. 2675, Seq. ID No. 2677, Seq. ID No. 2685, Seq. ID No. 2687, Seq. ID No. 2689, Seq. ID No. 2709, Seq. ID No. 2711, Seq. ID No. 2713, Seq. ID No. 2715, Seq. ID No. 2719, Seq. ID No. 2721, Seq. ID No. 2723, Seq. ID No. 2725, Seq. ID No. 2727, Seq. ID No. 2735, Seq. ID No. 2737, Seq. ID No. 2739, Seq. ID No. 2741, Seq. ID No. 2743, Seq. ID No. 2745, Seq. ID No. 2747, Seq. ID No. 2749, Seq. ID No. 2751, Seq. ID No. 2753, Seq. ID No. 2755, Seq. ID No. 2757, Seq. ID No. 2759, Seq. ID No. 2761, Seq. ID No. 2763, Seq. ID No. 2765, Seq. ID No. 2767, Seq. ID No. 2769, Seq. ID No. 2771, Seq. ID No. 2773, Seq. ID No. 2775, Seq. ID No. 2777, Seq. ID No. 2779, Seq. ID No. 2781, Seq. ID No. 2787, Seq. ID No. 2789, Seq. ID No. 2791, Seq. ID No. 2793, Seq. ID No. 2795, Seq. ID No. 2797, Seq. ID No. 2799, Seq. ID No. 2803, Seq. ID No. 2805, Seq. ID No. 2807, Seq. ID No. 2811, Seq. ID No. 2817, Seq. ID No. 2825, Seq. ID No. 2827, Seq. ID No. 2829, Seq. ID No. 2843, Seq. ID No. 2847, Seq. ID No. 2849, Seq. ID No. 2851, Seq. ID No. 2857, Seq. ID No. 2863, Seq. ID No. 2865, Seq. ID No. 2867, Seq. ID No. 2869, Seq. ID No. 2871, Seq. ID No. 2875, Seq. ID No. 2877, Seq. ID No. 2879, Seq. ID No. 2883, Seq. ID No. 2885, Seq. ID No. 2887, Seq. ID No. 2889, Seq. ID No. 2891, Seq. ID No. 2895, Seq. ID No. 2897, Seq. ID No. 2899, Seq. ID No. 2901, Seq. ID No. 2903, Seq. ID No. 2907, Seq. ID No. 2909, Seq. ID No. 2911, Seq. ID No. 2913, Seq. ID No. 2915, Seq. ID No. 2917, Seq. ID No. 2919, Seq. ID No. 2921, Seq. ID No. 2923, Seq. ID No. 2925, Seq. ID No. 2927, Seq. ID No. 2929, Seq. ID No. 2931, Seq. ID No. 2933, Seq. ID No. 2943, Seq. ID No. 2945, Seq. ID No. 2953, Seq. ID No. 2955, Seq. ID No. 2957, Seq. ID No. 2959, Seq. ID No. 2961, Seq. ID No. 2963, Seq. ID No. 2965, Seq. ID No. 2967, Seq. ID No. 2971, Seq. ID No. 2979, Seq. ID No. 2985, Seq. ID No. 2989, Seq. ID No. 2993, Seq. ID No. 2999, Seq. ID No. 3001, Seq. ID No. 3003, Seq. ID No. 3015, Seq. ID No. 3017, Seq. ID No. 3019, Seq. ID No. 3021, Seq. ID No. 3033, Seq. ID No. 3035, Seq. ID No. 3037, Seq. ID No. 3039, Seq. ID No. 3041, Seq. ID No. 3045, Seq. ID No. 3047, Seq. ID No. 3049, Seq. ID No. 3051, Seq. ID No. 3053, Seq. ID No. 3057, Seq. ID No. 3059, Seq. ID No. 3061, Seq. ID No. 3065, Seq. ID No. 3067, Seq. ID No. 3069, Seq. ID No. 3079, Seq. ID No. 3081, Seq. ID No. 3083, Seq. ID No. 3085, Seq. ID No. 3087, Seq. ID No. 3089, Seq. ID No. 3091, Seq. ID No. 3093, Seq. ID No. 3095, Seq. ID No. 3097, Seq. ID No. 3099, Seq. ID No. 3101, Seq. ID No. 3103, Seq. ID No. 3105, Seq. ID No. 3107, Seq. ID No. 3109, Seq. ID No. 3111, Seq. ID No. 3113, Seq. ID No. 3115, Seq. ID No. 3117, Seq. ID No. 3125, Seq. ID No. 3127, Seq. ID No. 3129, Seq. ID No. 3131, Seq. ID No. 3133, Seq. ID No. 3135, Seq. ID No. 3137, Seq. ID No. 3139, Seq. ID No. 3141, Seq. ID No. 3143, Seq. ID No. 3147, Seq. ID No. 3149, Seq. ID No. 3151, Seq. ID No. 3153, Seq. ID No. 3159, Seq. ID No. 3161, Seq. ID No. 3167, Seq. ID No. 3169, Seq. ID No. 3171, Seq. ID No. 3175, Seq. ID No. 3177, Seq. ID No. 3179, Seq. ID No. 3181, Seq. ID No. 3185, Seq. ID No. 3187, Seq. ID No. 3189, Seq. ID No. 3191, Seq. ID No. 3195, Seq. ID No. 3197, Seq. ID No. 3199, Seq. ID No. 3203, Seq. ID No. 3205, Seq. ID No. 3207, Seq. ID No. 3209, Seq. ID No. 3211, Seq. ID No. 3221, Seq. ID No. 3223, Seq. ID No. 3225, Seq. ID No. 3227, Seq. ID No. 3229, Seq. ID No. 3231, Seq. ID No. 3233, Seq. ID No. 3235, Seq. ID No. 3237, Seq. ID No. 3239, Seq. ID No. 3247, Seq. ID No. 3251, Seq. ID No. 3253, Seq. ID No. 3259, Seq. ID No. 3265, Seq. ID No. 3267, Seq. ID No. 3271, Seq. ID No. 3273, Seq. ID No. 3279, Seq. ID No. 3281, Seq. ID No. 3283, Seq. ID No. 3285, Seq. ID No. 3287, Seq. ID No. 3289, Seq. ID No. 3291, Seq. ID No. 3293, Seq. ID No. 3295, Seq. ID No. 3299, Seq. ID No. 3301, Seq. ID No. 3303, Seq. ID No. 3305, Seq. ID No. 3307, Seq. ID No. 3309, Seq. ID No. 3311, Seq. ID No. 3313, Seq. ID No. 3315, Seq. ID No. 3319, Seq. ID No. 3321, Seq. ID No. 3327, Seq. ID No. 3329, Seq. ID No. 3333, Seq. ID No. 3341, Seq. ID No. 3343, Seq. ID No. 3345, Seq. ID No. 3347, Seq. ID No. 3361, Seq. ID No. 3363, Seq. ID No. 3365, Seq. ID No. 3367, Seq. ID No. 3369, Seq. ID No. 3371, Seq. ID No. 3373, Seq. ID No. 3379, Seq. ID No. 3381, Seq. ID No. 3383, Seq. ID No. 3385, Seq. ID No. 3387, Seq. ID No. 3393, Seq. ID No. 3397, Seq. ID No. 3399, Seq. ID No. 3401, Seq. ID No. 3403, Seq. ID No. 3407, Seq. ID No. 3409, Seq. ID No. 3411, Seq. ID No. 3413, Seq. ID No. 3417, Seq. ID No. 3419, Seq. ID No. 3423, Seq. ID No. 3429, Seq. ID No. 3431, Seq. ID No. 3435, Seq. ID No. 3437, Seq. ID No. 3439, Seq. ID No. 3443, Seq. ID No. 3445, Seq. ID No. 3447, Seq. ID No. 3459, Seq. ID No. 3461, Seq. ID No. 3465, Seq. ID No. 3467, Seq. ID No. 3469, Seq. ID No. 3471, Seq. ID No. 3473, Seq. ID No. 3475, Seq. ID No. 3477, Seq. ID No. 3479, Seq. ID No. 3483, Seq. ID No. 3485, Seq. ID No. 3487, Seq. ID No. 3489, Seq. ID No. 3495, Seq. ID No. 3497, Seq. ID No. 3499, Seq. ID No. 3501, Seq. ID No. 3507, Seq. ID No. 3509, Seq. ID No. 3511, Seq. ID No. 3513, Seq. ID No. 3515, Seq. ID No. 3521, Seq. ID No. 3523, Seq. ID No. 3525, Seq. ID No. 3529, Seq. ID No. 3533, Seq. ID No. 3535, Seq. ID No. 3537, Seq. ID No. 3541, Seq. ID No. 3543, Seq. ID No. 3545, Seq. ID No. 3547, Seq. ID No. 3549, Seq. ID No. 3551, Seq. ID No. 3553, Seq. ID No. 3555, Seq. ID No. 3557, Seq. ID No. 3559, Seq. ID No. 3563, Seq. ID No. 3565, Seq. ID No. 3567, Seq. ID No. 3569, Seq. ID No. 3571, Seq. ID No. 3575, Seq. ID No. 3577, Seq. ID No. 3579, Seq. ID No. 3583, Seq. ID No. 3585, Seq. ID No. 3589, Seq. ID No. 3591, Seq. ID No. 3597, Seq. ID No. 3599, Seq. ID No. 3601, Seq. ID No. 3603,.Seq. ID No. 3605, Seq. ID No. 3607, Seq. ID No. 3609, Seq. ID No. 3613, Seq. ID No. 3615, Seq. ID No. 3623, Seq. ID No. 3627, Seq. ID No. 3631, Seq. ID No. 3633, Seq. ID No. 3637, Seq. ID No. 3639, Seq. ID No. 3641, Seq. ID No. 3643, Seq. ID No. 3645, Seq. ID No. 3647, Seq. ID No. 3649, Seq. ID No. 3651, Seq. ID No. 3653, Seq. ID No. 3655, Seq. ID No. 3657, Seq. ID No. 3659, Seq. ID No. 3661, Seq. ID No. 3663, Seq. ID No. 3665, Seq. ID No. 3667, Seq. ID No. 3669, Seq. ID No. 3671, Seq. ID No. 3673, Seq. ID No. 3675, Seq. ID No. 3681, Seq. ID No. 3683, Seq. ID No. 3685,Seq. ID No. 3687, Seq. ID No. 3691, Seq. ID No. 3693, Seq. ID No. 3695, Seq. ID No. 3697, Seq. ID No. 3699, Seq. ID No. 3701, Seq. ID No. 3703, Seq. ID No. 3705, Seq. ID No. 3707, Seq. ID No. 3709, Seq. ID No. 3711, Seq. ID No. 3713, Seq. ID No. 3715, Seq. ID No. 3719, Seq. ID No. 3721, Seq. ID No. 3725, Seq. ID No. 3729, Seq. ID No. 3731, Seq. ID No. 3733, Seq. ID No. 3735, Seq. ID No. 3737, Seq. ID No. 3739, Seq. ID No. 3743, Seq. ID No. 3747, Seq. ID No. 3749, Seq. ID No. 3751, Seq. ID No. 3759, Seq. ID No. 3767, Seq. ID No. 3769, Seq. ID No. 3771, Seq. ID No. 3773, Seq. ID No. 3775, Seq. ID No. 3777, Seq. ID No. 3779, Seq. ID No. 3785, Seq. ID No. 3787, Seq. ID No. 3789, Seq. ID No. 3791, Seq. ID No. 3795, Seq. ID No. 3797, Seq. ID No. 3801, Seq. ID No. 3803, Seq. ID No. 3805, Seq. ID No. 3809, Seq. ID No. 3811, Seq. ID No. 3819, Seq. ID No. 3821, Seq. ID No. 3823, Seq. ID No. 3825, Seq. ID No. 3827, Seq. ID No. 3829, Seq. ID No. 3831, Seq. ID No. 3833, Seq. ID No. 3835, Seq. ID No. 3841, Seq. ID No. 3843, Seq. ID No. 3845, Seq. ID No. 3847, Seq. ID No. 3855, Seq. ID No. 3857, Seq. ID No. 3867, Seq. ID No. 3869, Seq. ID No. 3871, Seq. ID No. 3873, Seq. ID No. 3875, Seq. ID No. 3877, Seq. ID No. 3881, Seq. ID No. 3883, Seq. ID No. 3885, Seq. ID No. 3887, Seq. ID No. 3893, Seq. ID No. 3897, Seq. ID No. 3899, Seq. ID No. 3901, Seq. ID No. 3905, Seq. ID No. 3907, Seq. ID No. 3909, Seq. ID No. 3911, Seq. ID No. 3913, Seq. ID No. 3917, Seq. ID No. 3919, Seq. ID No. 3921, Seq. ID No. 3927, Seq. ID No. 3929, Seq. ID No. 3931, Seq. ID No. 3933, Seq. ID No. 3935, Seq. ID No. 3937, Seq. ID No. 3939, Seq. ID No. 3941, Seq. ID No. 3943, Seq. ID No. 3945, Seq. ID No. 3947, Seq. ID No. 3949, Seq. ID No. 3951, Seq. ID No. 3953, Seq. ID No. 3955, Seq. ID No. 3957, Seq. ID No. 3961, Seq. ID No. 3965, Seq. ID No. 3967, Seq. ID No. 3969, Seq. ID No. 3971, Seq. ID No. 3977, Seq. ID No. 3979, Seq. ID No. 3981, Seq. ID No. 3983, Seq. ID No. 3985, Seq. ID No. 3987, Seq. ID No. 3989, Seq. ID No. 3991, Seq. ID No. 3999, Seq. ID No. 4001, Seq. ID No. 4003, Seq. ID No. 4011, Seq. ID No. 4013, Seq. ID No. 4015, Seq. ID No. 4017, Seq. ID No. 4019, Seq. ID No. 4021, Seq. ID No. 4023, Seq. ID No. 4029, Seq. ID No. 4031, Seq. ID No. 4033, Seq. ID No. 4035, Seq. ID No. 4037, Seq. ID No. 4051, Seq. ID No. 4053, Seq. ID No. 4055, Seq. ID No. 4057, Seq. ID No. 4059, Seq. ID No. 4063, Seq. ID No. 4065, Seq. ID No. 4067, Seq. ID No. 4069, Seq. ID No. 4075, Seq. ID No. 4077, Seq. ID No. 4085, Seq. ID No. 4087, Seq. ID No. 4089, Seq. ID No. 4091, Seq. ID No. 4093, Seq. ID No. 4095, Seq. ID No. 4097, Seq. ID No. 4099, Seq. ID No. 4105, Seq. ID No. 4107, Seq. ID No. 4109, Seq. ID No. 4113, Seq. ID No. 4115, Seq. ID No. 4117, Seq. ID No. 4119, Seq. ID No. 4121, Seq. ID No. 4123, Seq. ID No. 4125, Seq. ID No. 4127, Seq. ID No. 4129, Seq. ID No. 4131, Seq. ID No. 4147, Seq. ID No. 4149, Seq. ID No. 4155, Seq. ID No. 4157, Seq. ID No. 4159, Seq. ID No. 4161, Seq. ID No. 4163, Seq. ID No. 4165, Seq. ID No. 4167, Seq. ID No. 4169, Seq. ID No. 4171, Seq. ID No. 4173, Seq. ID No. 4175, Seq. ID No. 4177, Seq. ID No. 4179, Seq. ID No. 4183, Seq. ID No. 4185, Seq. ID No. 4187, Seq. ID No. 4189, Seq. ID No. 4191, Seq. ID No. 4193, Seq. ID No. 4195, Seq. ID No. 4197, Seq. ID No. 4199, Seq. ID No. 4201, Seq. ID No. 4203, Seq. ID No. 4205, Seq. ID No. 4207, Seq. ID No. 4209, Seq. ID No. 4211, Seq. ID No. 4213, Seq. ID No. 4215, Seq. ID No. 4217, Seq. ID No. 4219, Seq. ID No. 4229, Seq. ID No. 4235, Seq. ID No. 4237, Seq. ID No. 4239, Seq. ID No. 4243, Seq. ID No. 4245, Seq. ID No. 4249, Seq. ID No. 4251, Seq. ID No. 4255, Seq. ID No. 4267, Seq. ID No. 4271, Seq. ID No. 4273, Seq. ID No. 4277, Seq. ID No. 4279, Seq. ID No. 4281, Seq. ID No. 4283, Seq. ID No. 4289, Seq. ID No. 4291, Seq. ID No. 4293, Seq. ID No. 4295, Seq. ID No. 4299, Seq. ID No. 4301, Seq. ID No. 4303, Seq. ID No. 4305, Seq. ID No. 4309, Seq. ID No. 4315, Seq. ID No. 4317, Seq. ID No. 4319, Seq. ID No. 4321, Seq. ID No. 4323, Seq. ID No. 4325, Seq. ID No. 4327, Seq. ID No. 4329, Seq. ID No. 4331, Seq. ID No. 4333, Seq. ID No. 4335, Seq. ID No. 4337, Seq. ID No. 4339, Seq. ID No. 4341, Seq. ID No. 4345, Seq. ID No. 4347, Seq. ID No. 4349, Seq. ID No. 4351, Seq. ID No. 4353, Seq. ID No. 4355, Seq. ID No. 4357, Seq. ID No. 4359, Seq. ID No. 4361, Seq. ID No. 4363, Seq. ID No. 4365, Seq. ID No. 4367, Seq. ID No. 4369, Seq. ID No. 4371, Seq. ID No. 4373, Seq. ID No. 4375, Seq. ID No. 4377, Seq. ID No. 4381, Seq. ID No. 4383, Seq. ID No. 4387, Seq. ID No. 4393, Seq. ID No. 4395, Seq. ID No. 4397, Seq. ID No. 4401, Seq. ID No. 4405, Seq. ID No. 4407, Seq. ID No. 4409, Seq. ID No. 4411, Seq. ID No. 4413, Seq. ID No. 4415, Seq. ID No. 4417, Seq. ID No. 4419, Seq. ID No. 4421, Seq. ID No. 4423, Seq. ID No. 4425, Seq. ID No. 4427, Seq. ID No. 4429, Seq. ID No. 4431, Seq. ID No. 4433, Seq. ID No. 4435, Seq. ID No. 4443, Seq. ID No. 4445, Seq. ID No. 4447, Seq. ID No. 4449, Seq. ID No. 4451, Seq. ID No. 4453,.Seq. ID No. 4455, Seq. ID No. 4457, Seq. ID No. 4459, Seq. ID No. 4461, Seq. ID No. 4463, Seq. ID No. 4465, Seq. ID No. 4467, Seq. ID No. 4471, Seq. ID No. 4475, Seq. ID No. 4477, Seq. ID No. 4479, Seq. ID No. 4487, Seq. ID No. 4489, Seq. ID No. 4491, Seq. ID No. 4499, Seq. ID No. 4501, Seq. ID No. 4503, Seq. ID No. 4505, Seq. ID No. 4507, Seq. ID No. 4509, Seq. ID No. 4511, Seq. ID No. 4517, Seq. ID No. 4519, Seq. ID No. 4521, Seq. ID No. 4523, Seq. ID No. 4525, Seq. ID No. 4531, Seq. ID No. 4535, Seq. ID No. 4537, Seq. ID No. 4539, Seq. ID No. 4541, Seq. ID No. 4543, Seq. ID No. 4545, Seq. ID No. 4547, Seq. ID No. 4549, Seq. ID No. 4551, Seq. ID No. 4553, Seq. ID No. 4555, Seq. ID No. 4557, Seq. ID No. 4559, Seq. ID No. 4561, Seq. ID No. 4563, Seq. ID No. 4565, Seq. ID No. 4567, Seq. ID No. 4569, Seq. ID No. 4571, Seq. ID No. 4573, Seq. ID No. 4575, Seq. ID No. 4577, Seq. ID No. 4579, Seq. ID No. 4581, Seq. ID No. 4583, Seq. ID No. 4585, Seq. ID No. 4591, Seq. ID No. 4593, Seq. ID No. 4595, Seq. ID No. 4597, Seq. ID No. 4599, Seq. ID No. 4601, Seq. ID No. 4603, Seq. ID No. 4605, Seq. ID No. 4607, Seq. ID No. 4609, Seq. ID No. 4611, Seq. ID No. 4613, Seq. ID No. 4615, Seq. ID No. 4617, Seq. ID No. 4619, Seq. ID No. 4621, Seq. ID No. 4623, Seq. ID No. 4625, Seq. ID No. 4627, Seq. ID No. 4629, Seq. ID No. 4631, Seq. ID No. 4633, Seq. ID No. 4635, Seq. ID No. 4637, Seq. ID No. 4645, Seq. ID No. 4647, Seq. ID No. 4649, Seq. ID No. 4651, Seq. ID No. 4653, Seq. ID No. 4655, Seq. ID No. 4657, Seq. ID No. 4661, Seq. ID No. 4663, Seq. ID No. 4665, Seq. ID No. 4667, Seq. ID No. 4669, Seq. ID No. 4671, Seq. ID No. 4673, Seq. ID No. 4675, Seq. ID No. 4677, Seq. ID No. 4679, Seq. ID No. 4683, Seq. ID No. 4685, Seq. ID No. 4689, Seq. ID No. 4691, Seq. ID No. 4693, Seq. ID No. 4697, Seq. ID No. 4699, Seq. ID No. 4701, Seq. ID No. 4703, Seq. ID No. 4713, Seq. ID No. 4719, Seq. ID No. 4721, Seq. ID No. 4723, Seq. ID No. 4725, Seq. ID No. 4727, Seq. ID No. 4729, Seq. ID No. 4731, Seq. ID No. 4733, Seq. ID No. 4735, Seq. ID No. 4739, Seq. ID No. 4741, Seq. ID No. 4743, Seq. ID No. 4745, Seq. ID No. 4747, Seq. ID No. 4749, Seq. ID No. 4751, Seq. ID No. 4753, Seq. ID No. 4755, Seq. ID No. 4757, Seq. ID No. 4761, Seq. ID No. 4763, Seq. ID No. 4765, Seq. ID No. 4767, Seq. ID No. 4769, Seq. ID No. 4771, Seq. ID No. 4773, Seq. ID No. 4775, Seq. ID No. 4777, Seq. ID No. 4779, Seq. ID No. 4781, Seq. ID No. 4783, Seq. ID No. 4785, Seq. ID No. 4787, Seq. ID No. 4789, Seq. ID No. 4791, Seq. ID No. 4793, Seq. ID No. 4795, Seq. ID No. 4797, Seq. ID No. 4799, Seq. ID No. 4801, Seq. ID No. 4803, Seq. ID No. 4805, Seq. ID No. 4807, Seq. ID No. 4809, Seq. ID No. 4811, Seq. ID No. 4815, Seq. ID No. 4817, Seq. ID No. 4819, Seq. ID No. 4821, Seq. ID No. 4823, Seq. ID No. 4825, Seq. ID No. 4829, Seq. ID No. 4831, Seq. ID No. 4833, Seq. ID No. 4835, Seq. ID No. 4837, Seq. ID No. 4839, Seq. ID No. 4841, Seq. ID No. 4843, Seq. ID No. 4845, Seq. ID No. 4847, Seq. ID No. 4849, Seq. ID No. 4851, Seq. ID No. 4855, Seq. ID No. 4857, Seq. ID No. 4859, Seq. ID No. 4861, Seq. ID No. 4865, Seq. ID No. 4867, Seq. ID No. 4877, Seq. ID No. 4879, Seq. ID No. 4881, Seq. ID No. 4883, Seq. ID No. 4885, Seq. ID No. 4887, Seq. ID No. 4889, Seq. ID No. 4891, Seq. ID No. 4893, Seq. ID No. 4895, Seq. ID No. 4897, Seq. ID No. 4899, Seq. ID No. 4901, Seq. ID No. 4903, Seq. ID No. 4905, Seq. ID No. 4907, Seq. ID No. 4909, Seq. ID No. 4911, Seq. ID No. 4913, Seq. ID No. 4915, Seq. ID No. 4917, Seq. ID No. 4925, Seq. ID No. 4927, Seq. ID No. 4933, Seq. ID No. 4935, Seq. ID No. 4937, Seq. ID No. 4939, Seq. ID No. 4941, Seq. ID No. 4943, Seq. ID No. 4945, Seq. ID No. 4947, Seq. ID No. 4953, Seq. ID No. 4955, Seq. ID No. 4957, Seq. ID No. 4959, Seq. ID No. 4961, Seq. ID No. 4963, Seq. ID No. 4965, Seq. ID No. 4967, Seq. ID No. 4969, Seq. ID No. 4971, Seq. ID No. 4973, Seq. ID No. 4977, Seq. ID No. 4979, Seq. ID No. 4981, Seq. ID No. 4983, Seq. ID No. 4985, Seq. ID No. 4989, Seq. ID No. 4991, Seq. ID No. 4993, Seq. ID No. 4995, Seq. ID No. 49,97, Seq. ID No. 5001, Seq. ID No. 5003, Seq. ID No. 5005, Seq. ID No. 5007, Seq. ID No. 5009, Seq. ID No. 5011, Seq. ID No. 5015, Seq. ID No. 5017, Seq. ID No. 5019, Seq. ID No. 5021, Seq. ID No. 5023, Seq. ID No. 5025, Seq. ID No. 5027, Seq. ID No. 5029, Seq. ID No. 5033, Seq. ID No. 5035, Seq. ID No. 5037, Seq. ID No. 5039, Seq. ID No. 5041, Seq. ID No. 5043, Seq. ID No. 5045, Seq. ID No. 5047, Seq. ID No. 5049, Seq. ID No. 5051, Seq. ID No. 5053, Seq. ID No. 5055, Seq. ID No. 5057, Seq. ID No. 5059, Seq. ID No. 5065, Seq. ID No. 5067, Seq. ID No. 5071, Seq. ID No. 5073, Seq. ID No. 5075, Seq. ID No. 5077, Seq. ID No. 5079, Seq. ID No. 5081, Seq. ID No. 5083, Seq. ID No. 5087, Seq. ID No. 5089, Seq. ID No. 5091, Seq. ID No. 5093, Seq. ID No. 5095, Seq. ID No. 5097, Seq. ID No. 5099, Seq. ID No. 5101, Seq. ID No. 5105, Seq. ID No. 5107, Seq. ID No. 5109, Seq. ID No. 5111, Seq. ID No. 5113, Seq. ID No. 5117, Seq. ID No. 5119, Seq. ID No. 5121, Seq. ID No. 5127, Seq. ID No. 5143, Seq. ID No. 5145, Seq. ID No. 5147, Seq. ID No. 5149, Seq. ID No. 5151, Seq. ID No. 5153, Seq. ID No. 5155, Seq. ID No. 5157, Seq. ID No. 5161, Seq. ID No. 5163, Seq. ID No. 5167, Seq. ID No. 5169, Seq. ID No. 5171, Seq. ID No. 5173, Seq. ID No. 5177, Seq. ID No. 5179, Seq. ID No. 5181, Seq. ID No. 5183, Seq. ID No. 5185, Seq. ID No. 5187, Seq. ID No. 5189, Seq. ID No. 5191, Seq. ID No. 5193, Seq. ID No. 5195, Seq. ID No. 5201, Seq. ID No. 5203, Seq. ID No. 5209, Seq. ID No. 5211, Seq. ID No. 5215, Seq. ID No. 5217, Seq. ID No. 5221, Seq. ID No. 5225, Seq. ID No. 5227, Seq. ID No. 5229, Seq. ID No. 5231, Seq. ID No. 5233, Seq. ID No. 5235, Seq. ID No. 5237, Seq. ID No. 5239, Seq. ID No. 5241, Seq. ID No. 5243, Seq. ID No. 5245, Seq. ID No. 5247, Seq. ID No. 5249, Seq. ID No. 5251, Seq. ID No. 5253, Seq. ID No. 5255, Seq. ID No. 5257, Seq. ID No. 5259, Seq. ID No. 5261, Seq. ID No. 5263, Seq. ID No. 5265, Seq. ID No. 5269, Seq. ID No. 5271, Seq. ID No. 5283, Seq. ID No. 5287, Seq. ID No. 5289, Seq. ID No. 5291, Seq. ID No. 5293, Seq. ID No. 5295, Seq. ID No. 5297, Seq. ID No. 5299, Seq. ID No. 5301, Seq. ID No. 5303, Seq. ID No. 5305, Seq. ID No. 5307, Seq. ID No. 5309, Seq. ID No. 5311, Seq. ID No. 5313, Seq. ID No. 5315, Seq. ID No. 5317, Seq. ID No. 5319, Seq. ID No. 5321, Seq. ID No. 5323, Seq. ID No. 5325, Seq. ID No. 5333, Seq. ID No. 5335, Seq. ID No. 5337, Seq. ID No. 5339, Seq. ID No. 5343, Seq. ID No. 5345, Seq. ID No. 5347, Seq. ID No. 5349, Seq. ID No. 5351, Seq. ID No. 5353, Seq. ID No. 5355, Seq. ID No. 5357, Seq. ID No. 5359, Seq. ID No. 5361, Seq. ID No. 5363, Seq. ID No. 5365, Seq. ID No. 5367, Seq. ID No. 5369, Seq. ID No. 5371, Seq. ID No. 5373, Seq. ID No. 5375, Seq. ID No. 5381, Seq. ID No. 5383, Seq. ID No. 5389, Seq. ID No. 5391, Seq. ID No. 5393, Seq. ID No. 5395, Seq. ID No. 5397, Seq. ID No. 5399, Seq. ID No. 5401, Seq. ID No. 5403, Seq. ID No. 5407, Seq. ID No. 5409, Seq. ID No. 5411, Seq. ID No. 5417, Seq. ID No. 5419, Seq. ID No. 5421, Seq. ID No. 5423, Seq. ID No. 5425, Seq. ID No. 5427, Seq. ID No. 5429, Seq. ID No. 5431, Seq. ID No. 5435, Seq. ID No. 5437, Seq. ID No. 5439, Seq. ID No. 5441, Seq. ID No. 5443, Seq. ID No. 5445, Seq. ID No. 5447, Seq. ID No. 5449, Seq. ID No. 5451, Seq. ID No. 5455, Seq. ID No. 5457, Seq. ID No. 5467, Seq. ID No. 5469, Seq. ID No. 5471, Seq. ID No. 5473, Seq. ID No. 5475, Seq. ID No. 5477, Seq. ID No. 5479, Seq. ID No. 5481, Seq. ID No. 5483, Seq. ID No. 5485, Seq. ID No. 5487, Seq. ID No. 5489, Seq. ID No. 5493, Seq. ID No. 5495, Seq. ID No. 5499, Seq. ID No. 5501, Seq. ID No. 5503, Seq. ID No. 5505, Seq. ID No. 5507, Seq. ID No. 5511, Seq. ID No. 5519, Seq. ID No. 5521, Seq. ID No. 5523, Seq. ID No. 5527, Seq. ID No. 5535, Seq. ID No. 5537, Seq. ID No. 5539, Seq. ID No. 5541, Seq. ID No. 5547, Seq. ID No. 5549, Seq. ID No. 5551, Seq. ID No. 5553, Seq. ID No. 5555, Seq. ID No. 5557, Seq. ID No. 5559, Seq. ID No. 5561, Seq. ID No. 5563, Seq. ID No. 5565, Seq. ID No. 5567, Seq. ID No. 5571, Seq. ID No. 5573, Seq. ID No. 5575, Seq. ID No. 5577, Seq. ID No. 5579, Seq. ID No. 5581, Seq. ID No. 5583, Seq. ID No. 5585, Seq. ID No. 5599, Seq. ID No. 5601, Seq. ID No. 5603, Seq. ID No. 5605, Seq. ID No. 5607, Seq. ID No. 5613, Seq. ID No. 5615, Seq. ID No. 5617, Seq. ID No. 5619, Seq. ID No. 5627, Seq. ID No. 5629, Seq. ID No. 5631, Seq. ID No. 5635, Seq. ID No. 5645, Seq. ID No. 5647, Seq. ID No. 5651, Seq. ID No. 5653, Seq. ID No. 5655, Seq. ID No. 5657, Seq. ID No. 5661, Seq. ID No. 5663, Seq. ID No. 5665, Seq. ID No. 5669, Seq. ID No. 5671, Seq. ID No. 5673, Seq. ID No. 5675, Seq. ID No. 5677, Seq. ID No. 5679, Seq. ID No. 5691, Seq. ID No. 5693, Seq. ID No. 5695, Seq. ID No. 5697, Seq. ID No. 5701, Seq. ID No. 5707, Seq. ID No. 5713, Seq. ID No. 5715, Seq. ID No. 5717, Seq. ID No. 5721, Seq. ID No. 5723, Seq. ID No. 5725, Seq. ID No. 5727, Seq. ID No. 5731, Seq. ID No. 5737, Seq. ID No. 5741, Seq. ID No. 5743, Seq. ID No. 5745, Seq. ID No. 5747, Seq. ID No. 5749, Seq. ID No. 5751, Seq. ID No. 5753, Seq. ID No. 5755, Seq. ID No. 5757, Seq. ID No. 5759, Seq. ID No. 5761, Seq. ID No. 5769, Seq. ID No. 5773, Seq. ID No. 5775, Seq. ID No. 5777, Seq. ID No. 5779, Seq. ID No. 5781, Seq. ID No. 5787, Seq. ID No. 5789, Seq. ID No. 5793, Seq. ID No. 5795, Seq. ID No. 5799, Seq. ID No. 5801, Seq. ID No. 5803, Seq. ID No. 5805, Seq. ID No. 5807, Seq. ID No. 5809, Seq. ID No. 5811, Seq. ID No. 5813, Seq. ID No. 5815, Seq. ID No. 5817, Seq. ID No. 5819, Seq. ID No. 5821, Seq. ID No. 5823, Seq. ID No. 5825, Seq. ID No. 5827, Seq. ID No. 5831, Seq. ID No. 5833, Seq. ID No. 5835, Seq. ID No. 5837, Seq. ID No. 5839, Seq. ID No. 5841, Seq. ID No. 5843, Seq. ID No. 5845, Seq. ID No. 5847, Seq. ID No. 5849, Seq. ID No. 5851, Seq. ID No. 5853, Seq. ID No. 5855, Seq. ID No. 5857, Seq. ID No. 5859, Seq. ID No. 5861, Seq. ID No. 5863, Seq. ID No. 5865, Seq. ID No. 5867, Seq. ID No. 5869, Seq. ID No. 5871, Seq. ID No. 5873, Seq. ID No. 5875, Seq. ID No. 5877, Seq. ID No. 5881, Seq. ID No. 5887, Seq. ID No. 5891, Seq. ID No. 5897, Seq. ID No. 5903, Seq. ID No. 5907, Seq. ID No. 5911, Seq. ID No. 5913, Seq. ID No. 5915, Seq. ID No. 5917, Seq. ID No. 5919, Seq. ID No. 5925, Seq. ID No. 5931, Seq. ID No. 5933, Seq. ID No. 5935, Seq. ID No. 5937, Seq. ID No. 5941, Seq. ID No. 5943, Seq. ID No. 5945, Seq. ID No. 5947, Seq. ID No. 5949, Seq. ID No. 5951, Seq. ID No. 5953, Seq. ID No. 5955, Seq. ID No. 5957, Seq. ID No. 5959, Seq. ID No. 5961, Seq. ID No. 5963, Seq. ID No. 5965, Seq. ID No. 5967, Seq. ID No. 5969, Seq. ID No. 5971, Seq. ID No. 5973, Seq. ID No. 5975, Seq. ID No. 5977, Seq. ID No. 5979, Seq. ID No. 5981, Seq. ID No. 5983, Seq. ID No. 5985, Seq. ID No. 5987, Seq. ID No. 5989, Seq. ID No. 5991, Seq. ID No. 5993, Seq. ID No. 5995, Seq. ID No. 5997, Seq. ID No. 5999, Seq. ID No. 6001, Seq. ID No. 6003, Seq. ID No. 6005, Seq. ID No. 6007, Seq. ID No. 6009, Seq. ID No. 6011, Seq. ID No. 6013, Seq. ID No. 6015, Seq. ID No. 6017, Seq. ID No. 6019, Seq. ID No. 6021, Seq. ID No. 6029, Seq. ID No. 6031, Seq. ID No. 6033, Seq. ID No. 6035, Seq. ID No. 6037, Seq. ID No. 6039, Seq. ID No. 6041, Seq. ID No. 6045, Seq. ID No. 6049, Seq. ID No. 6051, Seq. ID No. 6055, Seq. ID No. 6057, Seq. ID No. 6061, Seq. ID No. 6065, Seq. ID No. 6069, Seq. ID No. 6071, Seq. ID No. 6073, Seq. ID No. 6075, Seq. ID No. 6077, Seq. ID No. 6079, Seq. ID No. 6081, Seq. ID No. 6083, Seq. ID No. 6085, Seq. ID No. 6093, Seq. ID No. 6097, Seq. ID No. 6099, Seq. ID No. 6101, Seq. ID No. 6105, Seq. ID No. 6113, Seq. ID No. 6119, Seq. ID No. 6121, Seq. ID No. 6123, Seq. ID No. 6125, Seq. ID No. 6127, Seq. ID No. 6129, Seq. ID No. 6131, Seq. ID No. 6133, Seq. ID No. 6139, Seq. ID No. 6141, Seq. ID No. 6143, Seq. ID No. 6145, Seq. ID No. 6147, Seq. ID No. 6149, Seq. ID No. 6151, Seq. ID No. 6153, Seq. ID No. 6155, Seq. ID No. 6157, Seq. ID No. 6167, Seq. ID No. 6171, Seq. ID No. 6173, Seq. ID No. 6175, Seq. ID No. 6177, Seq. ID No. 6179, Seq. ID No. 6181, Seq. ID No. 6193, Seq. ID No. 6195, Seq. ID No. 6199, Seq. ID No. 6201, Seq. ID No. 6207, Seq. ID No. 6211, Seq. ID No. 6213, Seq. ID No. 6215, Seq. ID No. 6219, Seq. ID No. 6231, Seq. ID No. 6233, Seq. ID No. 6239, Seq. ID No. 6247, Seq. ID No. 6251, Seq. ID No. 6255, Seq. ID No. 6257, Seq. ID No. 6261, Seq. ID No. 6269, Seq. ID No. 6271, Seq. ID No. 6277, Seq. ID No. 6279, Seq. ID No. 6281, Seq. ID No. 6287, Seq. ID No. 6291, Seq. ID No. 6293, Seq. ID No. 6295, Seq. ID No. 6297, Seq. ID No. 6299, Seq. ID No. 6305, Seq. ID No. 6307, Seq. ID No. 6309, Seq. ID No. 6311, Seq. ID No. 6313, Seq. ID No. 6315, Seq. ID No. 6317, Seq. ID No. 6319, Seq. ID No. 6321, Seq. ID No. 6323, Seq. ID No. 6329, Seq. ID No. 6331, Seq. ID No. 6333, Seq. ID No. 6335, Seq. ID No. 6337, Seq. ID No. 6339, Seq. ID No. 6341, Seq. ID No. 6343, Seq. ID No. 6345, Seq. ID No. 6347, Seq. ID No. 6349, Seq. ID No. 6351, Seq. ID No. 6353, Seq. ID No. 6355, Seq. ID No. 6357, Seq. ID No. 6359, Seq. ID No. 6361, Seq. ID No. 6367, Seq. ID No. 6369, Seq. ID No. 6371, Seq. ID No. 6373, Seq. ID No. 6375, Seq. ID No. 6379, Seq. ID No. 6385, Seq. ID No. 6387, Seq. ID No. 6389, Seq. ID No. 6391, Seq. ID No. 6393, Seq. ID No. 6395, Seq. ID No. 6397, Seq. ID No. 6399, Seq. ID No. 6401, Seq. ID No. 6403, Seq. ID No. 6405, Seq. ID No. 6407, Seq. ID No. 6409, Seq. ID No. 6411, Seq. ID No. 6413, Seq. ID No. 6417, Seq. ID No. 6419, Seq. ID No. 6421, Seq. ID No. 6423, Seq. ID No. 6433, Seq. ID No. 6435, Seq. ID No. 6437, Seq. ID No. 6439, Seq. ID No. 6441, Seq. ID No. 6443, Seq. ID No. 6445, Seq. ID No. 6447, Seq. ID No. 6449, Seq. ID No. 6451, Seq. ID No. 6453, Seq. ID No. 6455, Seq. ID No. 6457, Seq. ID No. 6459, Seq. ID No. 6461, Seq. ID No. 6463, Seq. ID No. 6465, Seq. ID No. 467, Seq. ID No. 6469, Seq. ID No. 6471, Seq. ID No. 6475, Seq. ID No. 6477, Seq. ID No. 6481, Seq. ID No. 6483, Seq. ID No. 6485, Seq. ID No. 6487, Seq. ID No. 6489, Seq. ID No. 6491, Seq. ID No. 6493, Seq. ID No. 6495, Seq. ID No. 6499, Seq. ID No. 6501, Seq. ID No. 6503, Seq. ID No. 6505, Seq. ID No. 6507, Seq. ID No. 6509, Seq. ID No. 6511, Seq. ID No. 6513, Seq. ID No. 6515, Seq. ID No. 6519, Seq. ID No. 6521, Seq. ID No. 6523, Seq. ID No. 6525, Seq. ID No. 6527, Seq. ID No. 6529, Seq. ID No. 6531, Seq. ID No. 6533, Seq. ID No. 6535, Seq. ID No. 6537, Seq. ID No. 6539, Seq. ID No. 6545, Seq. ID No. 6547, Seq. ID No. 6549, Seq. ID No. 6551, Seq. ID No. 6553, Seq. ID No. 6559, Seq. ID No. 6561, Seq. ID No. 6563, Seq. ID No. 6565, Seq. ID No. 6567, Seq. ID No. 6569, Seq. ID No. 6575, Seq. ID No. 6577, Seq. ID No. 6579, Seq. ID No. 6583, Seq. ID No. 6585, Seq. ID No. 6587, Seq. ID No. 6589, Seq. ID No. 6591, Seq. ID No. 6605, Seq. ID No. 6607, Seq. ID No. 6609, Seq. ID No. 6611, Seq. ID No. 6613, Seq. ID No. 6615, Seq. ID No. 6617, Seq. ID No. 6619, Seq. ID No. 6621, Seq. ID No. 6623, Seq. ID No. 6625, Seq. ID No. 6627, Seq. ID No. 6629, Seq. ID No. 6633, Seq. ID No. 6635, Seq. ID No. 6641, and Seq. ID No. 6647.

16. The polynucleotide of claim 7, wherein the polynucleotide encoding a polypeptide is identified by Pfam analysis, and the polynucleotide is selected from one of Seq. ID No. 89, Seq. ID No. 145, Seq. ID No. 155, Seq. ID No. 167, Seq. ID No. 173, Seq. ID No. 191, Seq. ID No. 215, Seq. ID No. 219, Seq. ID No. 221, Seq. ID No. 223, Seq. ID No. 225, Seq. ID No. 233, Seq. ID No. 239, Seq. ID No. 251, Seq. ID No. 253, Seq. ID No. 271, Seq. ID No. 291, Seq. ID No. 331, Seq. ID No. 341, Seq. ID No. 345, Seq. ID No. 375, Seq. ID No. 501, Seq. ID No. 505, Seq. ID No. 511, Seq. ID No. 529, Seq. ID No. 567, Seq. ID No. 569, Seq. ID No. 575, Seq. ID No. 593, Seq. ID No. 595, Seq. ID No. 601, Seq. ID No. 637, Seq. ID No. 649, Seq. ID No. 661, Seq. ID No. 669, Seq. ID No. 675, Seq. ID No. 681, Seq. ID No. 683, Seq. ID No. 729, Seq. ID No. 753, Seq. ID No. 779, Seq. ID No. 827, Seq. ID No. 839, Seq. ID No. 895, Seq. ID No. 939, Seq. ID No. 969, Seq. ID No. 985, Seq. ID No. 1015, Seq. ID No. 1039, Seq. ID No. 1075, Seq. ID No. 1137, Seq. ID No. 1155, Seq. ID No. 1163, Seq. ID No. 1175, Seq. ID No. 1223, Seq. ID No. 1231, Seq. ID No. 1237, Seq. ID No. 1241, Seq. ID No. 1261, Seq. ID No. 1331, Seq. ID No. 1339, Seq. ID No. 1369, Seq. ID No. 1477, Seq. ID No. 1517, Seq. ID No. 1531, Seq. ID No. 1539, Seq. ID No. 1549, Seq. ID No. 1563, Seq. ID No. 1567, Seq. ID No. 1571, Seq. ID No. 1573, Seq. ID No. 1589, Seq. ID No. 1595, Seq. ID No. 1647, Seq. ID No. 1657, Seq. ID No. 1771, Seq. ID No. 1785, Seq. ID No. 1789, Seq. ID No. 1819, Seq. ID No. 1821, Seq. ID No. 1969, Seq. ID No. 2015, Seq. ID No. 2047, Seq. ID No. 2087, Seq. ID No. 2089, Seq. ID No. 2093, Seq. ID No. 2115, Seq. ID No. 2117, Seq. ID No. 2127, Seq. ID No. 2141, Seq. ID No. 2179, Seq. ID No. 2207, Seq. ID No. 2223, Seq. ID No. 2253, Seq. ID No. 2275, Seq. ID No. 2301, Seq. ID No. 2315, Seq. ID No. 2329, Seq. ID No. 2343, Seq. ID No. 2349, Seq. ID No. 2363, Seq. ID No. 2381, Seq. ID No. 2391, Seq. ID No. 2417, Seq. ID No. 2421, Seq. ID No. 2423, Seq. ID No. 2429, Seq. ID No. 2481, Seq. ID No. 2487, Seq. ID No. 2489, Seq. ID No. 2491, Seq. ID No. 2509, Seq. ID No. 2519, Seq. ID No. 2525, Seq. ID No. 2547, Seq. ID No. 2553, Seq. ID No. 2563, Seq. ID No. 2587, Seq. ID No. 2599, Seq. ID No. 2607, Seq. ID No. 2613, Seq. ID No. 2651, Seq. ID No. 2675, Seq. ID No. 2681, Seq. ID No. 2687, Seq. ID No. 2693, Seq. ID No. 2699, Seq. ID No. 2703, Seq. ID No. 2709, Seq. ID No. 2711, Seq. ID No. 2727, Seq. ID No. 2753, Seq. ID No. 2819, Seq. ID No. 2821, Seq. ID No. 2823, Seq. ID No. 2867, Seq. ID No. 2871, Seq. ID No. 2907, Seq. ID No. 2919, Seq. ID No. 2957, Seq. ID No. 2971, Seq. ID No. 2979, Seq. ID No. 2999, Seq. ID No. 3005, Seq. ID No. 3019, Seq. ID No. 3035, Seq. ID No. 3065, Seq. ID No. 3085, Seq. ID No. 3149, Seq. ID No. 3165, Seq. ID No. 3183, Seq. ID No. 3203, Seq. ID No. 3211, Seq. ID No. 3251, Seq. ID No. 3259, Seq. ID No. 3319, Seq. ID No. 3323, Seq. ID No. 3335, Seq. ID No. 3337, Seq. ID No. 3347, Seq. ID No. 3359, Seq. ID No. 3377, Seq. ID No. 3379, Seq. ID No. 3399, Seq. ID No. 3425, Seq. ID No. 3483, Seq. ID No. 3495, Seq. ID No. 3497, Seq. ID No. 3519, Seq. ID No. 3529, Seq. ID No. 3599, Seq. ID No. 3611, Seq. ID No. 3615, Seq. ID No. 3619, Seq. ID No. 3663, Seq. ID No. 3739, Seq. ID No. 3759, Seq. ID No. 3771, Seq. ID No. 3797, Seq. ID No. 3825, Seq. ID No. 3841, Seq. ID No. 3855, Seq. ID No. 3863, Seq. ID No. 3901, Seq. ID No. 3929, Seq. ID No. 4015, Seq. ID No. 4017, Seq. ID No. 4055, Seq. ID No. 4075, Seq. ID No. 4085, Seq. ID No. 4089, Seq. ID No. 4099, Seq. ID No. 4105, Seq. ID No. 4129, Seq. ID No. 4203, Seq. ID No. 4243, Seq. ID No. 4251, Seq. ID No. 4343, Seq. ID No. 4383, Seq. ID No. 4475, Seq. ID No. 4481, Seq. ID No. 4487, Seq. ID No. 4491, Seq. ID No. 4533, Seq. ID No. 4627, Seq. ID No. 4705, Seq. ID No. 4713, Seq. ID No. 4717, Seq. ID No. 4719, Seq. ID No. 4729, Seq. ID No. 4789, Seq. ID No. 4791, Seq. ID No. 4793, Seq. ID No. 4803, Seq. ID No. 4813, Seq. ID No. 4815, Seq. ID No. 4825, Seq. ID No. 4833, Seq. ID No. 4837, Seq. ID No. 4875, Seq. ID No. 4879, Seq. ID No. 4919, Seq. ID No. 4931, Seq. ID No. 4947, Seq. ID No. 4985, Seq. ID No. 5077, Seq. ID No. 5115, Seq. ID No. 5117, Seq. ID No. 5127, Seq. ID No. 5141, Seq. ID No. 5193, Seq. ID No. 5215, Seq. ID No. 5219, Seq. ID No. 5275, Seq. ID No. 5381, Seq. ID No. 5389, Seq. ID No. 5409, Seq. ID No. 5411, Seq. ID No. 5475, Seq. ID No. 5491, Seq. ID No. 5513, Seq. ID No. 5519, Seq. ID No. 5525, Seq. ID No. 5529, Seq. ID No. 5537, Seq. ID No. 5557, Seq. ID No. 5581, Seq. ID No. 5599, Seq. ID No. 5637, Seq. ID No. 5685, Seq. ID No. 5723, Seq. ID No. 5755, Seq. ID No. 5761, Seq. ID No. 5895, Seq. ID No. 5901, Seq. ID No. 5903, Seq. ID No. 5911, Seq. ID No. 5913, Seq. ID No. 5919, Seq. ID No. 5923, Seq. ID No. 5929, Seq. ID No. 6001, Seq. ID No. 6043, Seq. ID No. 6053, Seq. ID No. 6055, Seq. ID No. 6089, Seq. ID No. 6095, Seq. ID No. 6111, Seq. ID No. 6117, Seq. ID No. 6165, Seq. ID No. 6169, Seq. ID No. 6187, Seq. ID No. 6195, Seq. ID No. 6219, Seq. ID No. 6233, Seq. ID No. 6269, Seq. ID No. 6275, Seq. ID No. 6279, Seq. ID No. 6287, Seq. ID No. 6297, Seq. ID No. 6299, Seq. ID No. 6305, Seq. ID No. 6311, Seq. ID No. 6369, Seq. ID No. 6379, Seq. ID No. 6587, and Seq. ID No. 6591

17. The polynucleotide of claim 7, wherein the polynucleotide encoding a polypeptide is identified by BlastP analysis, and the polynucleotide is selected from one of Seq. ID No. 9, Seq. ID No. 17, Seq. ID No. 45, Seq. ID No. 49, Seq. ID No. 53, Seq. ID No. 59, Seq. ID No. 89, Seq. ID No. 145, Seq. ID No. 157, Seq. ID No. 167, Seq. ID No. 171, Seq. ID No. 191, Seq. ID No. 215, Seq. ID No. 217, Seq. ID No. 225, Seq. ID No. 239, Seq. ID No. 253, Seq. ID No. 273, Seq. ID No. 277, Seq. ID No. 289, Seq. ID. No. 329, Seq. ID No. 343, Seq. ID No. 345, Seq. ID No. 375, Seq. ID No. 501, Seq. ID No. 505, Seq. ID No. 511, Seq. ID No. 515, Seq. ID No. 529, Seq. ID No. 569, Seq. ID No. 575, Seq. ID No. 593, Seq. ID No. 595, Seq. ID No. 637, Seq. ID No. 659, Seq. ID No. 669, Seq. ID No. 675, Seq. ID No. 681, Seq. ID No. 729, Seq. ID No. 779, Seq. ID No. 819, Seq. ID No. 895, Seq. ID No. 921, Seq. ID No. 935, Seq. ID No. 955, Seq. ID No. 967, Seq. ID No. 985, Seq. ID No. 1017, Seq. ID No. 1039, Seq. ID No. 1075, Seq. ID No. 1089, Seq. ID No. 1137, Seq. ID No. 1223, Seq. ID No. 1241, Seq. ID No. 1315, Seq. ID No. 1331, Seq. ID No. 1369, Seq. ID No. 1459, Seq. ID No. 1517, Seq. ID No. 1561, Seq. ID No. 1563, Seq. ID No. 1567, Seq. ID No. 1571, Seq. ID No. 1573, Seq. ID No. 1601, Seq. ID No. 1607, Seq. ID No. 1613, Seq. ID No. 1619, Seq. ID No. 1641, Seq. ID No. 1647, Seq. ID No. 1653, Seq. ID No. 1657, Seq. ID No. 1665, Seq. ID No. 1699, Seq. ID No. 1713, Seq. ID No. 1751, Seq. ID No. 1771, Seq. ID No. 1785, Seq. ID No. 1789, Seq. ID No. 1791, Seq. ID No. 1797, Seq. ID No. 1811, Seq. ID No. 1817, Seq. ID No. 1819, Seq. ID No. 1821, Seq. ID No. 1871, Seq. ID No. 1895, Seq. ID No. 1905, Seq. ID No. 1913, Seq. ID No. 1919, Seq. ID No. 1923, Seq. ID No. 1965, Seq. ID No. 1971, Seq. ID No. 1999, Seq. ID No. 2047, Seq. ID No. 2055, Seq. ID No. 2091, Seq. ID No. 2093, Seq. ID No. 2115, Seq. ID No. 2119, Seq. ID No. 2127, Seq. ID No. 2165, Seq. ID No. 2179, Seq. ID No. 2207, Seq. ID No. 2223, Seq. ID No. 2227, Seq. ID No. 2241, Seq. ID No. 2253, Seq. ID No. 2263, Seq. ID No. 2269, Seq. ID No. 2275, Seq. ID No. 2301, Seq. ID No. 2315, Seq. ID No. 2329, Seq. ID No. 2333, Seq. ID No. 2343, Seq. ID No. 2345, Seq. ID No. 2363, Seq. ID No. 2373, Seq. ID No. 2391, Seq. ID No. 2417, Seq. ID No. 2429, Seq. ID No. 2465, Seq. ID No. 2475, Seq. ID No. 2483, Seq. ID No. 2513, Seq. ID No. 2525, Seq. ID No. 2589, Seq. ID No. 2613, Seq. ID No. 2651, Seq. ID No. 2669, Seq. ID No. 2677, Seq. ID No. 2687, Seq. ID No. 2689, Seq. ID No. 2693, Seq. ID No. 2703, Seq. ID No. 2709, Seq. ID No. 2715, Seq. ID No. 2751, Seq. ID No. 2753, Seq. ID No. 2767, Seq. ID No. 2787, Seq. ID No. 2791, Seq. ID No. 2803, Seq. ID No. 2867, Seq. ID No. 2871, Seq. ID No. 2907, Seq. ID No. 2919, Seq. ID No. 2921, Seq. ID No. 2923, Seq. ID No. 2927, Seq. ID No. 2933, Seq. ID No. 2957, Seq. ID No. 2979, Seq. ID No. 2999, Seq. ID No. 3005, Seq. ID No. 3019, Seq. ID No. 3031, Seq. ID No. 3035, Seq. ID No. 3055, Seq. ID No. 3067, Seq. ID No. 3085, Seq. ID No. 3165, Seq. ID No. 3183, Seq. ID No. 3189, Seq. ID No. 3197, Seq. ID No. 3211, Seq. ID No. 3251, Seq. ID No. 3257, Seq. ID No. 3259, Seq. ID No. 3297, Seq. ID No. 3319, Seq. ID No. 3323, Seq. ID No. 3335, Seq. ID No. 3337, Seq. ID No. 3355, Seq. ID No. 3359, Seq. ID No. 3377, Seq. ID No. 3397, Seq. ID No. 3405, Seq. ID No. 3415, Seq. ID No. 3425, Seq. ID No. 3427, Seq. ID No. 3461, Seq. ID No. 3463, Seq. ID No. 3487, Seq. ID No. 3493, Seq. ID No. 3497, Seq. ID No. 3507, Seq. ID No. 3519, Seq. ID No. 3521, Seq. ID No. 3529, Seq. ID No. 3535, Seq. ID No. 3629, Seq. ID No. 3635, Seq. ID No. 3727, Seq. ID No. 3759, Seq. ID No. 3771, Seq. ID No. 3793, Seq. ID No. 3839, Seq. ID No. 3851, Seq. ID No. 3863, Seq. ID No. 3865, Seq. ID No. 3883, Seq. ID No. 3891, Seq. ID No. 3913, Seq. ID No. 3925, Seq. ID No. 3929, Seq. ID No. 3975, Seq. ID No. 3981, Seq. ID No. 4027, Seq. ID No. 4039, Seq. ID No. 4061, Seq. ID No. 4073, Seq. ID No. 4081, Seq. ID No. 4103, Seq. ID No. 4111, Seq. ID No. 4137, Seq. ID No. 4225, Seq. ID No. 4247, Seq. ID No. 4343, Seq. ID No. 4379, Seq. ID No. 4389, Seq. ID No. 4469, Seq. ID No. 4473, Seq. ID No. 4481, Seq. ID No. 4485, Seq. ID No. 4493, Seq. ID No. 4529, Seq. ID No. 4643, Seq. ID No. 4705, Seq. ID No. 4713, Seq. ID No. 4717, Seq. ID No. 4813, Seq. ID No. 4815, Seq. ID No. 4825, Seq. ID No. 4827, Seq. ID No. 4833, Seq. ID No. 4837, Seq. ID No. 4851, Seq. ID No. 4863, Seq. ID No. 4875, Seq. ID No. 4879, Seq. ID No. 4893, Seq. ID No. 4901, Seq. ID No. 4919, Seq. ID No. 4931, Seq. ID No. 4959, Seq. ID No. 4963, Seq. ID No. 4985, Seq. ID No. 5113, Seq. ID No. 5115, Seq. ID No. 5117, Seq. ID No. 5125, Seq. ID No. 5141, Seq. ID No. 5183, Seq. ID No. 5201, Seq. ID No. 5205, Seq. ID No. 5213, Seq. ID No. 5219, Seq. ID No. 5279, Seq. ID No. 5285, Seq. ID No. 5331, Seq. ID No. 5381, Seq. ID No. 5389, Seq. ID No. 5405, Seq. ID No. 5411, Seq. ID No. 5419, Seq. ID No. 5433, Seq. ID No. 5463, Seq. ID No. 5477, Seq. ID No. 5491, Seq. ID No. 5509, Seq. ID No. 5513, Seq. ID No. 5525, Seq. ID No. 5529, Seq. ID No. 5537, Seq. ID No. 5567, Seq. ID No. 5599, Seq. ID No. 5621, Seq. ID No. 5637, Seq. ID No. 5699, Seq. ID No. 5723, Seq. ID No. 5733, Seq. ID No. 5761, Seq. ID No. 5791, Seq. ID No. 5895, Seq. ID No. 5901, Seq. ID No. 5903, Seq. ID No. 5909, Seq. ID No. 5913, Seq. ID No. 5919, Seq. ID No. 5929, Seq. ID No. 5939, Seq. ID No. 6001, Seq. ID No. 6043, Seq. ID No. 6053, Seq. ID No. 6057, Seq. ID No. 6089, Seq. ID No. 6095, Seq. ID No. 6103, Seq. ID No. 6111, Seq. ID No. 6117, Seq. ID No. 6165, Seq. ID No. 6169, Seq. ID No. 6185, Seq. ID No. 6191, Seq. ID No. 6209, Seq. ID No. 6229, Seq. ID No. 6257, Seq. ID No. 6263, Seq. ID No. 6271, Seq. ID No. 6275, Seq. ID No. 6289, Seq. ID No. 6303, Seq. ID No. 6365, Seq. ID No. 6369, Seq. ID No. 6383, and Seq. ID No. 6415.

18. The polynucleotide of claim 7, wherein the polynucleotide has been identified using the proteomic methods used for studying surface exposed proteins of Streptococcus pneumoniae as encoding a polypeptide, and the polynucleotide is selected from one of Seq. ID No. 1, Seq. ID No. 5, Seq. ID No. 93, Seq. ID No. 95, Seq. ID No. 103, Seq. ID No. 107, Seq. ID No. 157, Seq. ID No. 223, Seq. ID No. 225, Seq. ID No. 233, Seq. ID No. 239, Seq. ID No. 253, Seq. ID No. 317, Seq. ID No. 321, Seq. ID No. 341, Seq. ID No. 345, Seq. ID No. 353, Seq. ID No. 361, Seq. ID No. 363, Seq. ID No. 365, Seq. ID No. 375, Seq. ID No. 379, Seq. ID No. 383, Seq. ID No. 389, Seq. ID No. 391, Seq. ID No. 397, Seq. ID No. 405, Seq. ID No. 409, Seq. ID No. 415, Seq. ID No. 423, Seq. ID No. 425, Seq. ID No. 427, Seq. ID No. 429, Seq. ID No. 435, Seq. ID No. 437, Seq. ID No. 439, Seq. ID No. 441, Seq. ID No. 443, Seq. ID No. 445, Seq. ID No. 447, Seq. ID No. 449, Seq. ID No. 461, Seq. ID No. 463, Seq. ID No. 465, Seq. ID No. 513, Seq. ID No. 543, Seq. ID No. 551, Seq. ID No. 553, Seq. ID No. 559, Seq. ID No. 595, Seq. ID No. 601, Seq. ID No. 637, Seq. ID No. 649, Seq. ID No. 657, Seq. ID No. 669, Seq. ID No. 675, Seq. ID No. 683, Seq. ID No. 753, Seq. ID No. 929, Seq. ID No. 969, Seq. ID No. 1041, Seq. ID No. 1043, Seq. ID No. 1085, Seq. ID No. 1089, Seq. ID No. 1093, Seq. ID No. 1097, Seq. ID No. 1099, Seq. ID No. 1101, Seq. ID No. 1175, Seq. ID No. 1205, Seq. ID No. 1237, Seq. ID No. 1459, Seq. ID No. 1495, Seq. ID No. 1497, Seq. ID No. 1503, Seq. ID No. 1517, Seq. ID No. 1527, Seq. ID No. 1531, Seq. ID No. 1539, Seq. ID No. 1543, Seq. ID No. 1549, Seq. ID No. 1573, Seq. ID No. 1657, Seq. ID No. 1709, Seq. ID No. 1767, Seq. ID No. 1785, Seq. ID No. 1821, Seq. ID No. 1889, Seq. ID No. 1925, Seq. ID No. 1969, Seq. ID No. 2003, Seq. ID No. 2047, Seq. ID No. 2075, Seq. ID No. 2089, Seq. ID No. 2093, Seq. ID No. 2117, Seq. ID No. 2137, Seq. ID No. 2143, Seq. ID No. 2205, Seq. ID No. 2235, Seq. ID No. 2257, Seq. ID No. 2301, Seq. ID No. 2363, Seq. ID No. 2423, Seq. ID No. 2445, Seq. ID No. 2451, Seq. ID No. 2481, Seq. ID No. 2495, Seq. ID No. 2505, Seq. ID No. 2577, Seq. ID No. 2587, Seq. ID No. 2613, Seq. ID No. 2633, Seq. ID No. 2651, Seq. ID No. 2665, Seq. ID No. 2669, Seq. ID No. 2677, Seq. ID No. 2687, Seq. ID No. 2693, Seq. ID No. 2699, Seq. ID No. 2711, Seq. ID No. 2715, Seq. ID No. 2757, Seq. ID No. 2791, Seq. ID No. 2793, Seq. ID No. 2803, Seq. ID No. 2867, Seq. ID No. 2919, Seq. ID No. 2929, Seq. ID No. 3005, Seq. ID No. 3019, Seq. ID No. 3125, Seq. ID No. 3137, Seq. ID No. 3161, Seq. ID No. 3179, Seq. ID No. 3251, Seq. ID No. 3259, Seq. ID No. 3285, Seq. ID No. 3309, Seq. ID No. 3317, Seq. ID No. 3337, Seq. ID No. 3345, Seq. ID No. 3359, Seq. ID No. 3475, Seq. ID No. 3515, Seq. ID No. 3521, Seq. ID No. 3641, Seq. ID No. 3695, Seq. ID No. 3793, Seq. ID No. 3901, Seq. ID No. 3927, Seq. ID No. 3987, Seq. ID No. 4001, Seq. ID No. 4057, Seq. ID No. 4081, Seq. ID No. 4111, Seq. ID No. 4125, Seq. ID No. 4193, Seq. ID No. 4195, Seq. ID No. 4237, Seq. ID No. 4247, Seq. ID No. 4383, Seq. ID No. 4393, Seq. ID No. 4397, Seq. ID No. 4401, Seq. ID No. 4435, Seq. ID No. 4449, Seq. ID No. 4471, Seq. ID No. 4491, Seq. ID No. 4511, Seq. ID No. 4519, Seq. ID No. 4531, Seq. ID No. 4549, Seq. ID No. 4577, Seq. ID No. 4581, Seq. ID No. 4585, Seq. ID No. 4609, Seq. ID No. 4611, Seq. ID No. 4613, Seq. ID No. 4675, Seq. ID No. 4685, Seq. ID No. 4719, Seq. ID No. 4729, Seq. ID No. 4743, Seq. ID No. 4755, Seq. ID No. 4781, Seq. ID No. 4787, Seq. ID No. 4837, Seq. ID No. 4841, Seq. ID No. 4849, Seq. ID No. 4855, Seq. ID No. 4889, Seq. ID No. 4901, Seq. ID No. 4915, Seq. ID No. 4917, Seq. ID No. 4931, Seq. ID No. 4935, Seq. ID No. 4977, Seq. ID No. 5025, Seq. ID No. 5029, Seq. ID No. 5109, Seq. ID No. 5115, Seq. ID No. 5117, Seq. ID No. 5155, Seq. ID No. 5185, Seq. ID No. 5201, Seq. ID No. 5203, Seq. ID No. 5219, Seq. ID No. 5261, Seq. ID No. 5289, Seq. ID No. 5295, Seq. ID No. 5377, Seq. ID No. 5389, Seq. ID No. 5399, Seq. ID No. 5411, Seq. ID No. 5537, Seq. ID No. 5621, Seq. ID No. 5627, Seq. ID No. 5657, Seq. ID No. 5675, Seq. ID No. 5717, Seq. ID No. 5777, Seq. ID No. 5859, Seq. ID No. 5901, Seq. ID No. 5913, Seq. ID No. 5919, Seq. ID No. 5925, Seq. ID No. 5933, Seq. ID No. 6055, Seq. ID No. 6129, Seq. ID No. 6133, Seq. ID No. 6187, Seq. ID No. 6193, Seq. ID No. 6219, Seq. ID No. 6271, Seq. ID No. 6277, Seq. ID No. 6369, Seq. ID No. 6375, Seq. ID No. 6379, Seq. ID No. 6389, Seq. ID No. 6591, and Seq. ID No. 6605.

19. The polynucleotide of claim 7, wherein the polypeptide encoded by the polynucleotide was identified using HMM LPXTG motif finder as having a LPXTG motif and wherein the polypeptide is covalently attached to the peptidoglycan layer, and the polynucleotide is selected from one of Seq. ID No. 503, Seq. ID No. 505, Seq. ID No. 1315, Seq. ID No. 1317, Seq. ID No. 1363, Seq. ID No. 1365, Seq. ID No. 1367, Seq. ID No. 1369, Seq. ID No. 1559, Seq. ID No. 1561, Seq. ID No. 1581, Seq. ID No. 1739, Seq. ID No. 1741, Seq. ID No. 2811, Seq. ID No. 2813, Seq. ID No. 3619, Seq. ID No. 5587, Seq. ID No. 5589, Seq. ID No. 5591, Seq. ID No. 5781, Seq. ID No. 5783, and Seq. ID No. 6103.

20. The polynucleotide of claim 7, wherein the polynucleotide encodes a lipoprotein as predicted by HMM Lipo software program, and the polynucleotide is selected from one of Seq. ID No. 45, Seq. ID No. 47, Seq. ID No. 171, Seq. ID No. 225, Seq. ID No. 419, Seq. ID No. 513, Seq. ID No. 575, Seq. ID No. 591, Seq. ID No. 593, Seq. ID No. 657, Seq. ID No. 659, Seq. ID No. 669, Seq. ID No. 675, Seq. ID No. 745, Seq. ID No. 747, Seq. ID No. 935, Seq. ID No. 1045, Seq. ID No. 1075, Seq. ID No. 1137, Seq. ID No. 1173, Seq. ID No. 1191, Seq. ID No. 1193, Seq. ID No. 1195, Seq. ID No. 1563, Seq. ID No. 1661, Seq. ID No. 1663, Seq. ID No. 1665, Seq. ID No. 1791, Seq. ID No. 1797, Seq. ID No. 1917, Seq. ID No. 1969, Seq. ID No. 2115, Seq. ID No. 2159, Seq. ID No. 2429, Seq. ID No. 2527, Seq. ID No. 2699, Seq. ID No. 2815, Seq. ID No. 2875, Seq. ID No. 2975, Seq. ID No. 2977, Seq. ID No. 2991, Seq. ID No. 3043, Seq. ID No. 3323, Seq. ID No. 3337, Seq. ID No. 4133, Seq. ID No. 4137, Seq. ID No. 4481, Seq. ID No. 4705, Seq. ID No. 4827, Seq. ID No. 4869, Seq. ID No. 5115, Seq. ID No. 5377, Seq. ID No. 5379, Seq. ID No. 5491, Seq. ID No. 5509, Seq. ID No. 5513, Seq. ID No. 5525, Seq. ID No. 5529, Seq. ID No. 5531, Seq. ID No. 5889, Seq. ID No. 5909, Seq. ID No. 6025, Seq. ID No. 6027, Seq. ID No. 6087, Seq. ID No. 6089, Seq. ID No. 6325, Seq. ID No. 6327, Seq. ID No. 6415, Seq. ID No. 6637, Seq. ID No. 6639, and Seq. ID No. 6645.

21. The polynucleotide of claim 7, wherein the polypeptide encoded by the polynucleotide is predicted by HMM program to be non-covalently bound to the peptidoglycan layer, and the polynucleotide is selected from one of Seq. ID No. 3589, Seq. ID No. 3987, Seq. ID No. 5219, and Seq. ID No. 5337.

22. The polynucleotide of claim 7, wherein the polynucleotide encodes a polypeptide having a RGDX motif wherein X is not a proline, and the polynucleotide is selected from one of Seq. ID No. 37, Seq. ID No. 99, Seq. ID No. 107, Seq. ID No. 109, Seq. ID No. 111, Seq. ID No. 351, Seq. ID No. 353, Seq. ID No. 465, Seq. ID No. 467, Seq. ID No. 469, Seq. ID No. 471, Seq. ID No. 683, Seq. ID No. 821, Seq. ID No. 823, Seq. ID No. 1021, Seq. ID No. 1023, Seq. ID No. 1157, Seq. ID No. 1159, Seq. ID No. 1233, Seq. ID No. 1333, Seq. ID No. 1335, Seq. ID No. 1449, Seq. ID No. 1559, Seq. ID No. 1561, Seq. ID No. 1563, Seq. ID No. 1625, Seq. ID No. 1777, Seq. ID No. 1827, Seq. ID No. 1829, Seq. ID No. 1871, Seq. ID No. 1925, Seq. ID No. 2135, Seq. ID No. 2145, Seq. ID No. 2173, Seq. ID No. 2175, Seq. ID No. 2177, Seq. ID No. 2179, Seq. ID No. 2259, Seq. ID No. 2317, Seq. ID No. 2319, Seq. ID No. 2321, Seq. ID No. 2323, Seq. ID No. 2325, Seq. ID No. 2327, Seq. ID No. 2329, Seq. ID No. 2401, Seq. ID No. 2671, Seq. ID No. 2863, Seq. ID No. 2869, Seq. ID No. 2919, Seq. ID No. 2921, Seq. ID No. 2993, Seq. ID No. 3087, Seq. ID No. 3137, Seq. ID No. 3279, Seq. ID No. 3281, Seq. ID No. 3283, Seq. ID No. 3333, Seq. ID No. 3335, Seq. ID No. 3497, Seq. ID No. 3499, Seq. ID No. 3501, Seq. ID No. 3533, Seq. ID No. 3671, Seq. ID No. 3673, Seq. ID No. 3675, Seq. ID No. 3837, Seq. ID No. 3839, Seq. ID No. 3841, Seq. ID No. 3843, Seq. ID No. 3929, Seq. ID No. 3961, Seq. ID No. 4037, Seq. ID No. 4193, Seq. ID No. 4239, Seq. ID No. 4327, Seq. ID No. 4329, Seq. ID No. 4333, Seq. ID No. 4393, Seq. ID No. 4463, Seq. ID No. 4465, Seq. ID No. 4467, Seq. ID No. 4597, Seq. ID No. 4629, Seq. ID No. 4631, Seq. ID No. 4675, Seq. ID No. 4677, Seq. ID No. 4679, Seq. ID No. 4689, Seq. ID No. 4691, Seq. ID No. 4693, Seq. ID No. 4781, Seq. ID No. 4799, Seq. ID No. 4801, Seq. ID No. 4803, Seq. ID No. 4861, Seq. ID No. 5009, Seq. ID No. 5143, Seq. ID No. 5145, Seq. ID No. 5179, Seq. ID No. 5181, Seq. ID No. 5183, Seq. ID No. 5249, Seq. ID No. 5251, Seq. ID No. 5253, Seq. ID No. 5259, Seq. ID No. 5261, Seq. ID No. 5293, Seq. ID No. 5295, Seq. ID No. 5297, Seq. ID No. 5299, Seq. ID No. 5307, Seq. ID No. 5309, Seq. ID No. 5411, Seq. ID No. 5535, Seq. ID No. 5537, Seq. ID No. 5745, Seq. ID No. 5821, Seq. ID No. 5823, Seq. ID No. 5825, Seq. ID No. 6029, Seq. ID No. 6171, Seq. ID No. 6307, Seq. ID No. 6309, and Seq. ID No. 6311.

23. The polynucleotide of claim 7, wherein the polynucleotide encodes a polypeptide that is predicted by BlastP analysis as being involved in capsule biosynthesis and transport, and the polynucleotide is selected from one of Seq. ID No. 49, Seq. ID No. 51, Seq. ID No. 53, Seq. ID No. 59, Seq. ID No. 195, Seq. ID No. 689, Seq. ID No. 703, Seq. ID No. 925, Seq. ID No. 1597, Seq. ID No. 1601, Seq. ID No. 1607, Seq. ID No. 1611, Seq. ID No. 1613, Seq. ID No. 1713, Seq. ID No. 2029, Seq. ID No. 2263, Seq. ID No. 2269, Seq. ID No. 2373, Seq. ID No. 2437, Seq. ID No. 2651, Seq. ID No. 2767, Seq. ID No. 2907, Seq. ID No. 3397, Seq. ID No. 3399, Seq. ID No. 3479, Seq. ID No. 3487, Seq. ID No. 3491, Seq. ID No. 3493, Seq. ID No. 3495, Seq. ID No. 3497, Seq. ID No. 3519, Seq. ID No. 3521, Seq. ID No. 3529, Seq. ID No. 3535, Seq. ID No. 3561, Seq. ID No. 3883, Seq. ID No. 3891, Seq. ID No. 3981, and Seq. ID No. 4087.

24. The polynucleotide of claim 7, wherein the polynucleotide encodes a polypeptide identified by BlastP analysis as being localized within the capsular loci of the Alloiococcus otitidis genome, and the polynucleotide is selected from one of Seq. ID No. 45, Seq. ID No. 47, Seq. ID No. 51, Seq. ID No. 53, Seq. ID No. 55, Seq. ID No. 57, Seq. ID No. 59, Seq. ID No. 683, Seq. ID No. 685, Seq. ID No. 687, Seq. ID No. 689, Seq. ID No. 691, Seq. ID No. 693, Seq. ID No. 695, Seq. ID No. 697, Seq. ID No. 699, Seq. ID No. 701, Seq. ID N. 703, Seq. ID No. 705, Seq. ID No. 707, Seq. ID No. 709, Seq. ID No. 711, Seq. ID No. 713, Seq. ID No. 715, Seq. ID No. 717, Seq. ID No. 719, Seq. ID No. 721, Seq. ID No. 723, Seq. ID No. 725, Seq. ID No. 727, Seq. ID No. 729, Seq. ID No. 731, Seq. ID No. 733, Seq. ID No. 735, Seq. ID No. 737, Seq. ID No. 739, Seq. ID No. 741, Seq. ID No. 743, Seq. ID No. 745, Seq. ID No. 747, Seq. ID No. 749, Seq. ID No. 751, Seq. ID No. 753, Seq. ID No. 3389, Seq. ID No. 3391, Seq. ID No. 3393, Seq. ID No. 3395, Seq. ID No. 3397, Seq. ID No. 3399, Seq. ID No. 3401, Seq. ID No. 3403, Seq. ID No. 3405, Seq. ID No. 3407, Seq. ID No. 3409, Seq. ID No. 3411, Seq. ID No. 3413, Seq. ID No. 3415, Seq. ID No. 3417, Seq. ID No. 3419, Seq. ID No. 3421, Seq. ID No. 3423, Seq. ID No. 3425, Seq. ID No. 3427, Seq. ID No. 3429, Seq. ID No. 3431, Seq. ID No. 3433, Seq. ID No. 3435, Seq. ID No. 3437, Seq. ID No. 3439, Seq. ID No. 3441, Seq. ID No. 3443, Seq. ID No. 3445, Seq. ID No. 3447, Seq. ID No. 3449, Seq. ID No. 3451, Seq. ID No. 3453, Seq. ID No. 3455, Seq. ID No. 3457, Seq. ID No. 3459, Seq. ID No. 3461, Seq. ID No. 3463, Seq. ID No. 3465, Seq. ID No. 3467, Seq. ID No. 3469, Seq. ID No. 3471, Seq. ID No. 3473, Seq. ID No. 3475, Seq. ID No. 3477, Seq. ID No. 3479, Seq. ID No. 3481, Seq. ID No. 3483, Seq. ID No. 3485, Seq. ID No. 3487, Seq. ID No. 3489, Seq. ID No. 3491, Seq. ID No. 3493, Seq. ID No. 3495, Seq. ID No. 3497, Seq. ID No. 3499, Seq. ID No. 3501, Seq. ID No. 3503, Seq. ID No. 3505, Seq. ID No. 3507, Seq. ID No. 3509, Seq. ID No. 3511, Seq. ID No. 3513, Seq. ID No. 3515, Seq. ID No. 3517, Seq. ID No. 3519, Seq. ID No. 3521, Seq. ID No. 3523, Seq. ID No. 3525, Seq. ID No. 3527, Seq. ID No. 3529, Seq. ID No. 3531, Seq. ID No. 3533, Seq. ID No. 3535, Seq. ID No. 3537, Seq. ID No. 3539, Seq. ID No. 3541, Seq. ID No. 3543, Seq. ID No. 3545, Seq. ID No. 3547, Seq. ID No. 3549, Seq. ID No. 3551, Seq. ID No. 3553, Seq. ID No. 3555, Seq. ID No. 3557, Seq. ID No. 3559, and Seq. ID No. 3561.

25. The polynucleotide of claim 7, wherein the polynucleotide encodes a polypeptide identified by BlastP analysis as being associated with sporulation, and the polynucleotide is selected from one of Seq. ID No. 1137, Seq. ID No. 1455, Seq. ID No. 2137, Seq. ID No. 2141, Seq. ID No. 2165, Seq. ID No. 2179, Seq. ID No. 2301, Seq. ID No. 2753, Seq. ID No. 4211, Seq. ID No. 4251, Seq. ID No. 4327, Seq. ID No. 4569, Seq. ID No. 4719, and Seq. ID No. 4729.

26. The polynucleotide of claim 7, wherein the polynucleotide is a unique ORF identified by BlastP analysis as having a BlastP ‘E’ value of >e⁻¹⁰, and the polynucleotide is selected from one of Seq. ID No. 7, Seq. ID No. 19, Seq. ID No. 21, Seq. ID No. 23, Seq. ID No. 31, Seq. ID No. 39, Seq. ID No. 65, Seq. ID No. 67, Seq. ID No. 69, Seq. ID No. 91, Seq. ID No. 99, Seq. ID No. 105, Seq. ID No. 113, Seq. ID No. 115, Seq. ID No. 123, Seq. ID No. 125, Seq. ID No. 127, Seq. ID No. 131, Seq. ID No. 133, Seq. ID No. 139, Seq. ID No. 151, Seq. ID No. 159, Seq. ID No. 161, Seq. ID No. 163, Seq. ID No. 165, Seq. ID No. 169, Seq. ID No. 185, Seq. ID No. 189, Seq. ID No. 193, Seq. ID No. 229, Seq. ID No. 231, Seq. ID No. 255, Seq. ID No. 257, Seq. ID No. 259, Seq. ID No. 275, Seq. ID No. 337, Seq. ID No. 339, Seq. ID No. 347, Seq. ID No. 349, Seq. ID No. 357, Seq. ID No. 359, Seq. ID No. 369, Seq. ID No. 371, Seq. ID No. 377, Seq. ID No. 385, Seq. ID No. 387, Seq. ID No. 393, Seq. ID No. 399, Seq. ID No. 401, Seq. ID No. 403, Seq. ID No. 407, Seq. ID No. 419, Seq. ID No. 431, Seq. ID No. 477, Seq. ID No. 495, Seq. ID No. 507, Seq. ID No. 519, Seq. ID No. 541, Seq. ID No. 545, Seq. ID No. 547, Seq. ID No. 549, Seq. ID No. 557, Seq. ID No. 589, Seq. ID No. 617, Seq. ID No. 619, Seq. ID No. 633, Seq. ID No. 639, Seq. ID No. 641, Seq. ID No. 643, Seq. ID No. 645, Seq. ID No. 647, Seq. ID No. 651, Seq. ID No. 653, Seq. ID No. 655, Seq. ID No. 667, Seq. ID No. 691, Seq. ID No. 693, Seq. ID No. 695, Seq. ID No. 697, Seq. ID No. 715, Seq. ID No. 717, Seq. ID No. 719, Seq. ID No. 731, Seq. ID No. 733, Seq. ID No. 741, Seq. ID No. 743, Seq. ID No. 745, Seq. ID No. 747, Seq. ID No. 749, Seq. ID No. 751, Seq. ID No. 755, Seq. ID No: 757, Seq. ID No. 759, Seq. ID No. 761, Seq. ID No. 763, Seq. ID No. 765, Seq. ID No. 767, Seq. ID No. 801, Seq. ID No. 803, Seq. ID No. 805, Seq. ID No. 807, Seq. ID No. 809, Seq. ID No. 811, Seq. ID No. 813, Seq. ID No. 833, Seq. ID No. 841, Seq. ID No. 849, Seq. ID No. 851, Seq. ID No. 853, Seq. ID No. 855, Seq. ID No. 887, Seq. ID No. 889, Seq. ID No. 891, Seq. ID No. 903, Seq. ID No. 905, Seq. ID No. 909, Seq. ID No. 937, Seq. ID No. 941, Seq. ID No. 943, Seq. ID No. 951, Seq. ID No. 957, Seq. ID No. 973, Seq. ID No. 975, Seq. ID No. 987, Seq. ID No. 1005, Seq. ID No. 1007, Seq. ID No. 1009, Seq. ID No. 1011, Seq. ID No. 1019, Seq. ID No. 1025, Seq. ID No. 1027, Seq. ID No. 1045, Seq. ID No. 1047, Seq. ID No. 1071, Seq. ID No. 1073, Seq. ID No. 1077, Seq. ID No. 1105, Seq. ID No. 1107, Seq. ID No. 1111, Seq. ID No. 1131, Seq. ID No. 1135, Seq. ID No. 1139, Seq. ID No. 1151, Seq. ID No. 1153, Seq. ID No. 1165, Seq. ID No. 1167, Seq. ID No. 1169, Seq. ID No. 1171, Seq. ID No. 1173, Seq. ID No. 1181, Seq. ID No. 1183, Seq. ID No. 1185, Seq. ID No. 1187, Seq. ID No. 1189, Seq. ID No. 1191, Seq. ID No. 1193, Seq. ID No. 1195, Seq. ID No. 1197, Seq. ID No. 1199, Seq. ID No. 1201, Seq. ID No. 1203, Seq. ID No. 1207, Seq. ID No. 1213, Seq. ID No. 1227, Seq. ID No. 1229, Seq. ID No. 1239, Seq. ID No. 1243, Seq. ID No. 1245, Seq. ID No. 1247, Seq. ID No. 1249, Seq. ID No. 1259, Seq. ID No. 1265, Seq. ID No. 1267, Seq. ID No. 1269, Seq. ID No. 1271, Seq. ID No. 1273, Seq. ID No. 1275, Seq. ID No. 1277, Seq. ID No. 1279, Seq. ID No. 1281, Seq. ID No. 1283, Seq. ID No. 1285, Seq. ID No. 1287, Seq. ID No. 1289, Seq. ID No. 1291, Seq. ID No. 1293, Seq. ID No. 1295, Seq. ID No. 1297, Seq. ID No. 1299, Seq. ID No. 1301, Seq. ID No. 1303, Seq. ID No. 1305, Seq. ID No. 1307, Seq. ID No. 1309, Seq. ID No. 1311, Seq. ID No. 1313, Seq. ID No. 1319, Seq. ID No. 1321, Seq. ID No. 1323, Seq. ID No. 1325, Seq. ID No. 1327, Seq. ID No. 1337, Seq. ID No. 1341, Seq. ID No. 1343, Seq. ID No. 1347, Seq. ID No. 1349, Seq. ID No. 1351, Seq. ID No. 1353, Seq. ID No. 1355, Seq. ID No. 1371, Seq. ID No. 1379, Seq. ID No. 1381, Seq. ID No. 1389, Seq. ID No. 1397, Seq. ID No. 1399, Seq. ID No. 1401, Seq. ID No. 1417, Seq. ID No. 1419, Seq. ID No. 1421, Seq. ID No. 1423, Seq. ID No. 1439, Seq. ID No. 1445, Seq. ID No. 1447, Seq. ID No. 1457, Seq. ID No. 1465, Seq. ID No. 1479, Seq. ID No. 1511, Seq. ID No. 1551, Seq. ID No. 1553, Seq. ID No. 1555, Seq. ID No. 1557, Seq. ID No. 1579, Seq. ID No. 1609, Seq. ID No. 1621, Seq. ID No. 1623, Seq. ID No. 1625, Seq. ID No. 1627, Seq. ID No. 1629, Seq. ID No. 1631, Seq. ID No. 1633, Seq. ID No. 1639, Seq. ID No. 1643, Seq. ID No. 1655, Seq. ID No. 1667, Seq. ID No. 1671, Seq. ID No. 1693, Seq. ID No. 1695, Seq. ID No. 1701, Seq. ID No. 1703, Seq. ID No. 1705, Seq. ID No. 1721, Seq. ID No. 1723, Seq. ID No. 1731, Seq. ID No. 1737, Seq. ID No. 1743, Seq. ID No. 1759, Seq. ID No. 1763, Seq. ID No. 1773, Seq. ID No. 1775, Seq. ID No. 1777, Seq. ID No. 1779, Seq. ID No. 1781, Seq. ID No. 1799, Seq. ID No. 1801, Seq. ID No. 1803, Seq. ID No. 1807, Seq. ID No. 1823, Seq. ID No. 1831, Seq. ID No. 1851, Seq. ID No. 1857, Seq. ID No. 1875, Seq. ID No. 1899, Seq. ID No. 1915, Seq. ID No. 1927, Seq. ID No. 1929, Seq. ID No. 1973, Seq. ID No. 1977, Seq. ID No. 1981, Seq. ID No. 2005, Seq. ID No. 2007, Seq. ID No. 2009, Seq. ID No. 2011, Seq. ID No. 2017, Seq. ID No. 2019, Seq. ID No. 2023, Seq. ID No. 2025, Seq. ID No. 2027, Seq. ID No. 2039, Seq. ID No. 2041, Seq. ID No. 2043, Seq. ID No. 2045, Seq. ID No. 2049, Seq. ID No. 2081, Seq. ID No. 2083, Seq. ID No. 2085, Seq. ID No. 2105, Seq. ID No. 2107, Seq. ID No. 2109, Seq. ID No. 2111, Seq. ID No. 2129, Seq. ID No. 2131, Seq. ID No. 2133, Seq. ID No. 2145, Seq. ID No. 2155, Seq. ID No. 2157, Seq. ID No. 2159, Seq. ID No. 2161, Seq. ID No. 2171, Seq. ID No. 2187, Seq. ID No. 2189, Seq. ID No. 2201, Seq. ID No. 2203, Seq. ID No. 2209, Seq. ID No. 2211, Seq. ID No. 2219, Seq. ID No. 2225, Seq. ID No. 2243, Seq. ID No. 2245, Seq. ID No. 2247, Seq. ID No. 2249, Seq. ID No. 2251, Seq. ID No. 2255, Seq. ID No. 2277, Seq. ID No. 2279, Seq. ID No. 2341, Seq. ID No. 2351, Seq. ID No. 2353, Seq. ID No. 2355, Seq. ID No. 2357, Seq. ID No. 2359, Seq. ID No. 2365, Seq. ID No. 2367, Seq. ID No. 2371, Seq. ID No. 2375, Seq. ID No. 2393, Seq. ID No. 2395, Seq. ID No. 2425, Seq. ID No. 2427, Seq. ID No. 2431, Seq. ID No. 2439, Seq. ID No. 2441, Seq. ID No. 2443, Seq. ID No. 2447, Seq. ID No. 2459, Seq. ID No. 2479, Seq. ID No. 2493, Seq. ID No. 2501, Seq. ID No. 2503, Seq. ID No. 2521, Seq. ID No. 2523, Seq. ID No. 2527, Seq. ID No. 2591, Seq. ID No. 2601, Seq. ID No. 2603, Seq. ID No. 2611, Seq. ID No. 2627, Seq. ID No. 2629, Seq. ID No. 2653, Seq. ID No. 2661, Seq. ID No. 2691, Seq. ID No. 2695, Seq. ID No. 2697, Seq. ID No. 2717, Seq. ID No. 2729, Seq. ID No. 2731, Seq. ID No. 2733, Seq. ID No. 2735, Seq. ID No. 2739, Seq. ID No. 2741, Seq. ID No. 2743, Seq. ID No. 2777, Seq. ID No. 2779, Seq. ID No. 2781, Seq. ID No. 2783, Seq. ID No. 2801, Seq. ID No. 2805, Seq. ID No. 2807, Seq. ID No. 2809, Seq. ID No. 2811, Seq. ID No. 2813, Seq. ID No. 2815, Seq. ID No. 2817, Seq. ID No. 2819, Seq. ID No. 2829, Seq. ID No. 2831, Seq. ID No. 2833, Seq. ID No. 2835, Seq. ID No. 2837, Seq. ID No. 2839, Seq. ID No. 2841, Seq. ID No. 2845, Seq. ID No. 2849, Seq. ID No. 2851, Seq. ID No. 2853, Seq. ID No. 2855, Seq. ID No. 2857, Seq. ID No. 2859, Seq. ID No. 2861, Seq. ID No. 2865, Seq. ID No. 2873, Seq. ID No. 2875, Seq. ID No. 2877, Seq. ID No. 2883, Seq. ID No. 2885, Seq. ID No. 2887, Seq. ID No. 2889, Seq. ID No. 2893, Seq. ID No. 2895, Seq. ID No. 2903, Seq. ID No. 2909, Seq. ID No. 2935, Seq. ID No. 2937, Seq. ID No. 2939, Seq. ID No. 2941, Seq. ID No. 2943, Seq. ID No. 2945, Seq. ID No. 2947, Seq. ID No. 2949, Seq. ID No. 2951, Seq. ID No. 2953, Seq. ID No. 2965, Seq. ID No. 2967, Seq. ID No. 2973, Seq. ID No. 2975, Seq. ID No. 2977, Seq. ID No. 2981, Seq. ID No. 2983, Seq. ID No. 2985, Seq. ID No. 2987, Seq. ID No. 2989, Seq. ID No. 2991, Seq. ID No. 3001, Seq. ID No. 3007, Seq. ID No. 3009, Seq. ID No. 3011, Seq. ID No. 3021, Seq. ID No. 3023, Seq. ID No. 3041, Seq. ID No. 3045, Seq. ID No. 3071, Seq. ID No. 3087, Seq. ID No. 3105, Seq. ID No. 3107, Seq. ID No. 3119, Seq. ID No. 3121, Seq. ID No. 3123, Seq. ID No. 3151, Seq. ID No. 3153, Seq. ID No. 3167, Seq. ID No. 3173, Seq. ID No. 3175, Seq. ID No. 3177, Seq. ID No. 3181, Seq. ID No. 3193, Seq. ID No. 3213, Seq. ID No. 3215, Seq. ID No. 3217, Seq. ID No. 3219, Seq. ID No. 3221, Seq. ID No. 3223, Seq. ID No. 3231, Seq. ID No. 3233, Seq. ID No. 3235, Seq. ID No. 3237, Seq. ID No. 3239, Seq. ID No. 3241, Seq. ID No. 3243, Seq. ID No. 3245, Seq. ID No. 3247, Seq. ID No. 3249, Seq. ID No. 3261, Seq. ID No. 3263, Seq. ID No. 3265, Seq. ID No. 3267, Seq. ID No. 3269, Seq. ID No. 3271, Seq. ID No. 3273, Seq. ID No. 3275, Seq. ID No. 3277, Seq. ID No. 3325, Seq. ID No. 3331, Seq. ID No. 3339, Seq. ID No. 3341, Seq. ID No. 3349, Seq. ID No. 3361, Seq. ID No. 3363, Seq. ID No. 3369, Seq. ID No. 3371, Seq. ID No. 3389, Seq. ID No. 3403, Seq. ID No. 3417, Seq. ID No. 3419, Seq. ID No. 3421, Seq. ID No. 3433, Seq. ID No. 3437, Seq. ID No. 3441, Seq. ID No. 3443, Seq. ID No. 3445, Seq. ID No. 3447, Seq. ID No. 3473, Seq. ID No. 3481, Seq. ID No. 3483, Seq. ID No. 3489, Seq. ID No. 3503, Seq. ID No. 3505, Seq. ID No. 3509, Seq. ID No. 3511, Seq. ID No. 3523, Seq. ID No. 3525, Seq. ID No. 3531, Seq. ID No. 3533, Seq. ID No. 3539, Seq. ID No. 3541, Seq. ID No. 3543, Seq. ID No. 3545, Seq. ID No. 3547, Seq. ID No. 3555, Seq. ID No. 3557, Seq. ID No. 3563, Seq. ID No. 3565, Seq. ID No. 3571, Seq. ID No. 3573, Seq. ID No. 3581, Seq. ID No. 3587, Seq. ID No. 3591, Seq. ID No. 3593, Seq. ID No. 3595, Seq. ID No. 3611, Seq. ID No. 3613, Seq. ID No. 3617, Seq. ID No. 3619, Seq. ID No. 3621, Seq. ID No. 3623, Seq. ID No. 3625, Seq. ID No. 3631, Seq. ID No. 3637, Seq. ID No. 3639, Seq. ID No. 3655, Seq. ID No. 3657, Seq. ID No. 3659, Seq. ID No. 3661, Seq. ID No. 3677, Seq. ID No. 3679, Seq. ID No. 3683, Seq. ID No. 3685, Seq. ID No. 3701, Seq. ID No. 3703, Seq. ID No. 3717, Seq. ID No. 3719, Seq. ID No. 3721, Seq. ID No. 3723, Seq. ID No. 3729, Seq. ID No. 3731, Seq. ID No. 3733, Seq. ID No. 3735, Seq. ID No. 3737, Seq. ID No. 3739, Seq. ID No. 3741, Seq. ID No. 3749, Seq. ID No. 3751, Seq. ID No. 3753, Seq. ID No. 3755, Seq. ID No. 3761, Seq. ID No. 3781, Seq. ID No. 3783, Seq. ID No. 3785, Seq. ID No. 3787, Seq. ID No. 3789, Seq. ID No. 3791, Seq. ID No. 3799, Seq. ID No. 3807, Seq. ID No. 3809, Seq. ID No. 3811, Seq. ID No. 3813, Seq. ID No. 3815, Seq. ID No. 3817, Seq. ID No. 3827, Seq. ID No. 3829, Seq. ID No. 3831, Seq. ID No. 3833, Seq. ID No. 3835, Seq. ID No. 3849, Seq. ID No. 3879, Seq. ID No. 3881, Seq. ID No. 3895, Seq. ID No. 3903, Seq. ID No. 3909, Seq. ID No. 3915, Seq. ID No. 3939, Seq. ID No. 3959, Seq. ID No. 3961, Seq. ID No. 3963, Seq. ID No. 3965, Seq. ID No. 3973, Seq. ID No. 3977, Seq. ID No. 3979, Seq. ID No. 3993, Seq. ID No. 3995, Seq. ID No. 3997, Seq. ID No. 4005, Seq. ID No. 4007, Seq. ID No. 4009, Seq. ID No. 4041, Seq. ID No. 4043, Seq. ID No. 4045, Seq. ID No. 4069, Seq. ID No. 4077, Seq. ID No. 4097, Seq. ID No. 4113, Seq. ID No. 4123, Seq. ID No. 4131, Seq. ID No. 4133, Seq. ID No. 4135, Seq. ID No. 4139, Seq. ID No. 4151, Seq. ID No. 4153, Seq. ID No. 4155, Seq. ID No. 4181, Seq. ID No. 4221, Seq. ID No. 4241, Seq. ID No. 4253, Seq. ID No. 4257, Seq. ID No. 4259, Seq. ID No. 4261, Seq. ID No. 4263, Seq. ID No. 4265, Seq. ID No. 4269, Seq. ID No. 4275, Seq. ID No. 4277, Seq. ID No. 4279, Seq. ID No. 4281, Seq. ID No. 4285, Seq. ID No. 4287, Seq. ID No. 4297, Seq. ID No. 4305, Seq. ID No. 4307, Seq. ID No. 4363, Seq. ID No. 4365, Seq. ID No. 4371, Seq. ID No. 4373, Seq. ID No. 4395, Seq. ID No. 4399, Seq. ID No. 4403, Seq. ID No. 4425, Seq. ID No. 4437, Seq. ID No. 4439, Seq. ID No. 4441, Seq. ID No. 4453, Seq. ID No. 4455, Seq. ID No. 4513, Seq. ID No. 4515, Seq. ID No. 4551, Seq. ID No. 4561, Seq. ID No. 4573, Seq. ID No. 4575, Seq. ID No. 4587, Seq. ID No. 4589, Seq. ID No. 4591, Seq. ID No. 4593, Seq. ID No. 4595, Seq. ID No. 4617, Seq. ID No. 4619, Seq. ID No. 4621, Seq. ID No. 4625, Seq. ID No. 4627, Seq. ID No. 4633, Seq. ID No. 4635, Seq. ID No. 4637, Seq. ID No. 4639, Seq. ID No. 4659, Seq. ID No. 4681, Seq. ID No. 4687, Seq. ID No. 4695, Seq. ID No. 4699, Seq. ID No. 4701, Seq. ID No. 4721, Seq. ID No. 4735, Seq. ID No. 4737, Seq. ID No. 4741, Seq. ID No. 4783, Seq. ID No. 4789, Seq. ID No. 4805, Seq. ID No. 4811, Seq. ID No. 4817, Seq. ID No. 4819, Seq. ID No. 4821, Seq. ID No. 4823, Seq. ID No. 4835, Seq. ID No. 4853, Seq. ID No. 4885, Seq. ID No. 4913, Seq. ID No. 4923, Seq. ID No. 4949, Seq. ID No. 4951, Seq. ID No. 4975, Seq. ID No. 4981, Seq. ID No. 4983, Seq. ID No. 4987, Seq. ID No. 4993, Seq. ID No. 4999, Seq. ID No. 5001, Seq. ID No. 5011, Seq. ID No. 5013, Seq. ID No. 5023, Seq. ID No. 5031, Seq. ID No. 5033, Seq. ID No. 5035, Seq. ID No. 5037, Seq. ID No. 5041, Seq. ID No. 5043, Seq. ID No. 5053, Seq. ID No. 5061, Seq. ID No. 5063, Seq. ID No. 5085, Seq. ID No. 5093, Seq. ID No. 5151, Seq. ID No. 5153, Seq. ID No. 5159, Seq. ID No. 5161, Seq. ID No. 5167, Seq. ID No. 5173, Seq. ID No. 5175, Seq. ID No. 5177, Seq. ID No. 5189, Seq. ID No. 5191, Seq. ID No. 5193, Seq. ID No. 5197, Seq. ID No. 5199, Seq. ID No. 5221, Seq. ID No. 5223, Seq. ID No. 5225, Seq. ID No. 5227, Seq. ID No. 5237, Seq. ID No. 5267, Seq. ID No. 5269, Seq. ID No. 5271, Seq. ID No. 5281, Seq. ID No. 5301, Seq. ID No. 5303, Seq. ID No. 5315, Seq. ID No. 5321, Seq. ID No. 5327, Seq. ID No. 5347, Seq. ID No. 5385, Seq. ID No. 5445, Seq. ID No. 5447, Seq. ID No. 5459, Seq. ID No. 5465, Seq. ID No. 5467, Seq. ID No. 5479, Seq. ID No. 5481, Seq. ID No. 5485, Seq. ID No. 5487, Seq. ID No. 5515, Seq. ID No. 5517, Seq. ID No. 5533, Seq. ID No. 5543, Seq. ID No. 5545, Seq. ID No. 5549, Seq. ID No. 5569, Seq. ID No. 5577, Seq. ID No. 5593, Seq. ID No. 5595, Seq. ID No. 5609, Seq. ID No. 5623, Seq. ID No. 5625, Seq. ID No. 5639, Seq. ID No. 5641, Seq. ID No. 5643, Seq. ID No. 5649, Seq. ID No. 5659, Seq. ID No. 5661, Seq. ID No. 5663, Seq. ID No. 5667, Seq. ID No. 5671, Seq. ID No. 5687, Seq. ID No. 5689, Seq. ID No. 5703, Seq. ID No. 5705, Seq. ID No. 5709, Seq. ID No. 5711, Seq. ID No. 5737, Seq. ID No. 5739, Seq. ID No. 5745, Seq. ID No. 5751, Seq. ID No. 5763, Seq. ID No. 5797, Seq. ID No. 5811, Seq. ID No. 5813, Seq. ID No. 5815, Seq. ID No. 5827, Seq. ID No. 5829, Seq. ID No. 5843, Seq. ID No. 5845, Seq. ID No. 5873, Seq. ID No. 5879, Seq. ID No. 5883, Seq. ID No. 5885, Seq. ID No. 5887, Seq. ID No. 5889, Seq. ID No. 5891, Seq. ID No. 5931, Seq. ID No. 5955, Seq. ID No. 5963, Seq. ID No. 5965, Seq. ID No. 5975, Seq. ID No. 5983, Seq. ID No. 5985, Seq. ID No. 5987, Seq. ID No. 5989, Seq. ID No. 6005, Seq. ID No. 6013, Seq. ID No. 6031, Seq. ID No. 6035, Seq. ID No. 6045, Seq. ID No. 6059, Seq. ID No. 6061, Seq. ID No. 6063, Seq. ID No. 6073, Seq. ID No. 6081, Seq. ID No. 6091, Seq. ID No. 6093, Seq. ID No. 6127, Seq. ID No. 6159, Seq. ID No. 6197, Seq. ID No. 6199, Seq. ID No. 6203, Seq. ID No. 6217, Seq. ID No. 6221, Seq. ID No. 6223, Seq. ID No. 6237, Seq. ID No. 6239, Seq. ID No. 6241, Seq. ID No. 6245, Seq. ID No. 6247, Seq. ID No. 6249, Seq. ID No. 6259, Seq. ID No. 6265, Seq. ID No. 6267, Seq. ID No. 6273, Seq. ID No. 6283, Seq. ID No. 6285, Seq. ID No. 6323, Seq. ID No. 6325, Seq. ID No. 6327, Seq. ID No. 6361, Seq. ID No. 6363, Seq. ID No. 6377, Seq. ID No. 6381, Seq. ID No. 6401, Seq. ID No. 6403, Seq. ID No. 6419, Seq. ID No. 6425, Seq. ID No. 6427, Seq. ID No. 6429, Seq. ID No. 6431, Seq. ID No. 6433, Seq. ID No. 6435, Seq. ID No. 6437, Seq. ID No. 6439, Seq. ID No. 6447, Seq. ID No. 6449, Seq. ID No. 6451, Seq. ID No. 6453, Seq. ID No. 6455, Seq. ID No. 6457, Seq. ID No. 6465, Seq. ID No. 6467, Seq. ID No. 6469, Seq. ID No. 6471, Seq. ID No. 6473, Seq. ID No. 6475, Seq. ID No. 6477, Seq. ID No. 6479, Seq. ID No. 6481, Seq. ID No. 6483, Seq. ID No. 6485, Seq. ID No. 6487, Seq. ID No. 6489, Seq. ID No. 6491, Seq. ID No. 6495, Seq. ID No. 6497, Seq. ID No. 6507, Seq. ID No. 6517, Seq. ID No. 6519, Seq. ID No. 6521, Seq. ID No. 6523, Seq. ID No. 6525, Seq. ID No. 6527, Seq. ID No. 6529, Seq. ID No. 6533, Seq. ID No. 6539, Seq. ID No. 6541, Seq. ID No. 6543, Seq. ID No. 6555, Seq. ID No. 6557, Seq. ID No. 6561, Seq. ID No. 6563, Seq. ID No. 6565, Seq. ID No. 6567, Seq. ID No. 6569, Seq. ID No. 6571, Seq. ID No. 6573, Seq. ID No. 6581, Seq. ID No. 6583, Seq. ID No. 6631, Seq. ID No. 6635, Seq. ID No. 6637, Seq. ID No. 6639, Seq. ID No. 6643, Seq. ID No. 6645, Seq. ID No. 6647, and Seq. ID No. 6649.

27. The polynucleotide of claim 7, wherein the polypeptide encoded by the polynucleotide is identified by GLIMMER™ ORF finder program, and the polynucleotide is selected from one of Seq. ID No. 1, Seq. ID No. 5, Seq. ID No. 11, Seq. ID No. 13, Seq. ID No. 15, Seq. ID No. 17, Seq. ID No. 25, Seq. ID No. 27, Seq. ID No. 29, Seq. ID No. 35, Seq. ID No. 37, Seq. ID No. 41, Seq. ID No. 43, Seq. ID No. 47, Seq. ID No. 51, Seq. ID No. 53, Seq. ID No. 59, Seq. ID No. 63, Seq. ID No. 69, Seq. ID No. 73, Seq. ID No. 75, Seq. ID No. 81, Seq. ID No. 83, Seq. ID No. 89, Seq. ID No. 93, Seq. ID No. 97, Seq. ID No. 103, Seq. ID No. 111, Seq. ID No. 121, Seq. ID No. 125, Seq. ID No. 129, Seq. ID No. 135, Seq. ID No. 137, Seq. ID No. 145, Seq. ID No. 149, Seq. ID No. 157, Seq. ID No. 167, Seq. ID No. 173, Seq. ID No. 183, Seq. ID No. 187, Seq. ID No. 189, Seq. ID No. 191, Seq. ID No. 195, Seq. ID No. 197, Seq. ID No. 203, Seq. ID No. 207, Seq. ID No. 215, Seq. ID No. 219, Seq. ID No. 223, Seq. ID No. 225, Seq. ID No. 227, Seq. ID No. 231, Seq. ID No. 233, Seq. ID No. 239, Seq. ID No. 241, Seq. ID No. 247, Seq. ID No. 253, Seq. ID No. 261, Seq. ID No. 265, Seq. ID No. 269, Seq. ID No. 273, Seq. ID No. 275, Seq. ID No. 277, Seq. ID No. 283, Seq. ID No. 291, Seq. ID No. 293, Seq. ID No. 297, Seq. ID No. 301, Seq. ID No. 307, Seq. ID No. 309, Seq. ID No. 313, Seq. ID No. 317, Seq. ID No. 321, Seq. ID No. 325, Seq. ID No. 335, Seq. ID No. 343, Seq. ID No. 345, Seq. ID No. 353, Seq. ID No. 355, Seq. ID No. 361, Seq. ID No. 363, Seq. ID No. 367, Seq. ID No. 375, Seq. ID No. 379, Seq. ID No. 383, Seq. ID No. 389, Seq. ID No. 391, Seq. ID No. 397, Seq. ID No. 405, Seq. ID No. 411, Seq. ID No. 415, Seq. ID No. 417, Seq. ID No. 423, Seq. ID No. 425, Seq. ID No. 427, Seq. ID No. 429, Seq. ID No. 435, Seq. ID No. 437, Seq. ID No. 439, Seq. ID No. 441, Seq. ID No. 445, Seq. ID No. 447, Seq. ID No. 453, Seq. ID No. 461, Seq. ID No. 463, Seq. ID No. 471, Seq. ID No. 485, Seq. ID No. 501, Seq. ID No. 505, Seq. ID No. 511, Seq. ID No. 515, Seq. ID No. 517, Seq. ID No. 521, Seq. ID No. 523, Seq. ID No. 531, Seq. ID No. 533, Seq. ID No. 539, Seq. ID No. 541, Seq. ID No. 543, Seq. ID No. 551, Seq. ID No. 555, Seq. ID No. 561, Seq. ID No. 565, Seq. ID No. 569, Seq. ID No. 575, Seq. ID No. 579, Seq. ID No. 587, Seq. ID No. 593, Seq. ID No. 597, Seq. ID No. 603, Seq. ID No. 605, Seq. ID No. 609, Seq. ID No. 611, Seq. ID No. 613, Seq. ID No. 615, Seq. ID No. 623, Seq. ID No. 627, Seq. ID No. 629, Seq. ID No. 631, Seq. ID No. 635, Seq. ID No. 637, Seq. ID No. 647, Seq. ID No. 649, Seq. ID No. 661, Seq. ID No. 669, Seq. ID No. 673, Seq. ID No. 675, Seq. ID No. 681, Seq. ID No. 683, Seq. ID No. 689, Seq. ID No. 701, Seq. ID No. 703, Seq. ID No. 709, Seq. ID No. 713, Seq. ID No. 729, Seq. ID No. 733, Seq. ID No. 735, Seq. ID No. 737, Seq. ID No. 741, Seq. ID No. 747, Seq. ID No. 751, Seq. ID No. 753, Seq. ID No. 759, Seq. ID No. 765, Seq. ID No. 767, Seq. ID No. 771, Seq. ID No. 781, Seq. ID No. 783, Seq. ID No. 787, Seq. ID No. 791, Seq. ID No. 795, Seq. ID No. 799, Seq. ID No. 807, Seq. ID No. 817, Seq. ID No. 819, Seq. ID No. 823, Seq. ID No. 827, Seq. ID No. 831, Seq. ID No. 835, Seq. ID No. 837, Seq. ID No. 839, Seq. ID No. 847, Seq. ID No. 851, Seq. ID No. 855, Seq. ID No. 859, Seq. ID No. 867, Seq. ID No. 871, Seq. ID No. 875, Seq. ID No. 881, Seq. ID No. 885, Seq. ID No. 889, Seq. ID No. 891, Seq. ID No. 895, Seq. ID No. 901, Seq. ID No. 907, Seq. ID No. 915, Seq. ID No. 919,Seq. ID No. 925, Seq. ID No. 929, Seq. ID No. 935, Seq. ID No. 939, Seq. ID No. 949, Seq. ID No. 951, Seq. ID No. 955, Seq. ID No. 961, Seq. ID No. 965, Seq. ID No. 967, Seq. ID No. 971, Seq. ID No. 985, Seq. ID No. 993, Seq. ID No. 997, Seq. ID No. 1003, Seq. ID No. 1005, Seq. ID No. 1009, Seq. ID No. 1017, Seq. ID No. 1023, Seq. ID No. 1039, Seq. ID No. 1041, Seq. ID No. 1043, Seq. ID No. 1051, Seq. ID No. 1053, Seq. ID No. 1057, Seq. ID No. 1061, Seq. ID No. 1065, Seq. ID No. 1067, Seq. ID No. 1069, Seq. ID No. 1073, Seq. ID No. 1075, Seq. ID No. 1083, Seq. ID No. 1087, Seq. ID No. 1091, Seq. ID No. 1093, Seq. ID No. 1097, Seq. ID No. 1099, Seq. ID No. 1103, Seq. ID No. 1109, Seq. ID No. 1115, Seq. ID No.1117, Seq. ID No. 1123, Seq. ID No. 1129, Seq. ID No. 1133, Seq. ID No. 1137, Seq. ID No. 1143, Seq. ID No. 1147, Seq. ID No. 1149, Seq. ID No. 1155, Seq. ID No. 1157, Seq. ID No. 1159, Seq. ID No. 1163, Seq. ID No. 1171, Seq. ID No. 1179, Seq. ID No. 1181, Seq. ID No. 1187, Seq. ID No. 1193, Seq. ID No. 1199, Seq. ID No. 1205, Seq. ID No. 1211, Seq. ID No. 1217, Seq. ID No. 1221, Seq. ID No. 1223, Seq. ID No. 1225, Seq. ID No. 1231, Seq. ID No. 1233, Seq. ID No. 1237, Seq. ID No. 1241, Seq. ID No. 1253, Seq. ID No. 1257, Seq. ID No. 1263, Seq. ID No. 1271, Seq. ID No. 1281, Seq. ID No. 1317, Seq. ID No. 1323, Seq. ID No. 1331, Seq. ID No. 1335, Seq. ID No. 1339, Seq. ID No. 1345, Seq. ID No. 1347, Seq. ID No. 1361, Seq. ID No. 1369, Seq. ID No. 1377, Seq. ID No. 1379, Seq. ID No. 1387, Seq. ID No. 1393, Seq. ID No. 1395, Seq. ID No. 1399, Seq. ID No. 1403, Seq. ID No. 1405, Seq. ID No. 1411, Seq. ID No. 1413, Seq. ID No. 1415, Seq. ID No. 1419, Seq. ID No. 1425, Seq. ID No. 1427, Seq. ID No. 1429, Seq. ID No. 1431, Seq. ID No. 1433, Seq. ID No. 1437, Seq. ID No. 1443, Seq. ID No. 1449, Seq. ID No. 1451, Seq. ID No. 1455, Seq. ID No. 1457, Seq. ID No. 1459, Seq. ID No. 1463, Seq. ID No. 1465, Seq. ID No. 1469, Seq. ID No. 1471, Seq. ID No. 1473, Seq. ID No. 1475, Seq. ID No. 1477, Seq. ID No. 1485, Seq. ID No. 1487, Seq. ID No. 1491, Seq. ID No. 1493, Seq. ID No. 1495, Seq. ID No. 1497, Seq. ID No. 1501, Seq. ID No. 1505, Seq. ID No. 1507, Seq. ID No. 1509, Seq. ID No. 1517, Seq. ID No. 1525, Seq. ID No. 1527, Seq. ID No. 1531, Seq. ID No. 1539, Seq. ID No. 1541, Seq. ID No. 1543, Seq. ID No. 1549, Seq. ID No. 1561, Seq. ID No. 1563, Seq. ID No. 1567, Seq. ID No. 1571, Seq. ID No. 1573, Seq. ID No. 1577, Seq. ID No. 1581, Seq. ID No. 1589, Seq. ID No. 1593, Seq. ID No. 1595, Seq. ID No. 1597, Seq. ID No. 1603, Seq. ID No. 1607, Seq. ID No. 1611, Seq. ID No. 1613, Seq. ID No. 1619, Seq. ID No. 1623, Seq. ID No. 1627, Seq. ID No. 1631, Seq. ID No. 1637, Seq. ID No. 1639, Seq. ID No. 1641, Seq. ID No. 1647, Seq. ID No. 1653, Seq. ID No. 1657, Seq. ID No. 1665, Seq. ID No. 1669, Seq. ID No. 1681, Seq. ID No. 1685, Seq. ID No. 1689, Seq. ID No. 1691, Seq. ID No. 1697, Seq. ID No. 1699, Seq. ID No. 1707, Seq. ID No. 1711, Seq. ID No. 1713, Seq. ID No. 1719, Seq. ID No. 1723, Seq. ID No. 1729, Seq. ID No. 1735, Seq. ID No. 1737, Seq. ID No. 1741, Seq. ID No. 1747, Seq. ID No. 1751, Seq. ID No. 1757, Seq. ID No. 1759, Seq. ID No. 1761, Seq. ID No. 1771, Seq. ID No. 1777, Seq. ID No. 1785, Seq. ID No. 1789, Seq. ID No. 1791, Seq. ID No. 1797, Seq. ID No. 1801, Seq. ID No. 1803, Seq. ID No. 1805, Seq. ID No. 1811, Seq. ID No. 1817, Seq. ID No. 1819, Seq. ID No. 1821, Seq. ID No. 1825, Seq. ID No. 1829, Seq. ID No. 1837, Seq. ID No. 1843, Seq. ID No. 1845, Seq. ID No. 1847, Seq. ID No. 1849, Seq. ID No. 1851, Seq. ID No. 1855, Seq. ID No. 1859, Seq. ID No. 1863, Seq. ID No. 1865, Seq. ID No. 1867, Seq. ID No. 1871, Seq. ID No. 1873, Seq. ID No. 1877, Seq. ID No. 1885, Seq. ID No. 1887, Seq. ID No. 1889, Seq. ID No. 1895, Seq. ID No. 1897, Seq. ID No. 1899, Seq. ID No. 1903, Seq. ID No. 1905, Seq. ID No. 1913, Seq. ID No. 1919, Seq. ID No. 1923, Seq. ID No. 1925, Seq. ID No. 1931, Seq. ID No. 1935, Seq. ID No. 1937, Seq. ID No. 1943, Seq. ID No. 1945, Seq. ID No. 1949, Seq. ID No. 1953, Seq. ID No. 1957, Seq. ID No. 1959, Seq. ID No. 1961, Seq. ID No. 1965, Seq. ID No. 1967, Seq. ID No. 1971, Seq. ID No. 1975, Seq. ID No. 1979, Seq. ID No. 1983, Seq. ID No. 1987, Seq. ID No. 1989, Seq. ID No. 1991, Seq. ID No. 1999, Seq. ID No. 2001, Seq. ID No. 2003, Seq. ID No. 2009, Seq. ID No. 2015, Seq. ID No. 2019, Seq. ID No. 2021, Seq. ID No. 2031, Seq. ID No. 2035, Seq. ID No. 2037, Seq. ID No. 2045, Seq. ID No. 2047, Seq. ID No. 2055, Seq. ID No. 2059, Seq. ID No. 2065, Seq. ID No. 2067, Seq. ID No. 2069, Seq. ID No. 2071, Seq. ID No. 2073, Seq. ID No. 2079, Seq. ID No. 2091, Seq. ID No. 2093, Seq. ID No. 2097, Seq. ID No. 2099, Seq. ID No. 2103, Seq. ID No. 2115, Seq. ID No. 2119, Seq. ID No. 2127, Seq. ID No. 2135, Seq. ID No. 2137, Seq. ID No. 2141, Seq. ID No. 2143, Seq. ID No. 2147, Seq. ID No. 2153, Seq. ID No. 2159, Seq. ID No. 2161, Seq. ID No. 2165, Seq. ID No. 2169, Seq. ID No. 2179, Seq. ID No. 2183, Seq. ID No. 2185, Seq. ID No. 2189, Seq. ID No. 2191, Seq. ID No. 2193, Seq. ID No. 2195, Seq. ID No. 2199, Seq. ID No. 2207, Seq. ID No. 2217, Seq. ID No. 2219, Seq. ID No. 9921, Seq. ID No. 2223, Seq. ID No. 2227, Seq. ID No. 2229, Seq. ID No. 2231, Seq. ID No. 2233, Seq. ID No. 2241, Seq. ID No. 2245, Seq. ID No. 2251, Seq. ID No. 2253, Seq. ID No. 2257, Seq. ID No. 2259, Seq. ID No. 9963, Seq. ID No. 2267, Seq. ID No. 2269, Seq. ID No. 2275, Seq. ID No. 2281, Seq. ID No. 2287, Seq. ID No. 2297, Seq. ID No. 2299, Seq. ID No. 2301, Seq. ID No. 2315, Seq. ID No. 2329, Seq. ID No. 2333, Seq. ID No. 2335, Seq. ID No. 2339, Seq. ID No. 2343, Seq. ID No. 2345, Seq. ID No. 2349, Seq. ID No. 2351, Seq. ID No. 2357, Seq. ID No. 2359, Seq. ID No. 2363, Seq. ID No. 2365, Seq. ID No. 2369, Seq. ID No. 2373, Seq. ID No. 2381, Seq. ID No. 2391, Seq. ID No. 2399, Seq. ID No. 2401, Seq. ID No. 2405, Seq. ID No. 2409, Seq. ID No. 2415, Seq. ID No. 2417, Seq. ID No. 2421, Seq. ID No. 2423, Seq. ID No. 2427, Seq. ID No. 2429, Seq. ID No. 2431, Seq. ID No. 2435, Seq. ID No. 2437, Seq. ID No. 2439, Seq. ID No. 2443, Seq. ID No. 2445, Seq. ID No. 2449, Seq. ID No. 2451, Seq. ID No. 2453, Seq. ID No. 2457, Seq. ID No. 2463, Seq. ID No. 2465, Seq. ID No. 2469, Seq. ID No. 2471, Seq. ID No. 2475, Seq. ID No. 2477, Seq. ID No. 2483, Seq. ID No. 2485, Seq. ID No. 2491, Seq. ID No. 2495, Seq. ID No. 2505, Seq. ID No. 2507, Seq. ID No. 2513, Seq. ID No. 2519, Seq. ID No. 2525, Seq. ID No. 2529, Seq. ID No. 2535, Seq. ID No. 2537, Seq. ID No. 2539, Seq. ID No. 2549, Seq. ID No. 2553, Seq. ID No. 2563, Seq. ID No. 2567, Seq. ID No. 2575, Seq. ID No. 2577, Seq. ID No. 2581, Seq. ID No. 2589, Seq. ID No. 2593, Seq. ID No. 2595, Seq. ID No. 2599, Seq. ID No. 2603, Seq. ID No. 2607, Seq. ID No. 2609, Seq. ID No. 2613, Seq. ID No. 2617, Seq. ID No. 2619, Seq. ID No. 2623, Seq. ID No. 2625, Seq. ID No. 2631, Seq. ID No. 2633, Seq. ID No. 2639, Seq. ID No. 2641, Seq. ID No. 2645, Seq. ID No. 2649, Seq. ID No. 2651, Seq. ID No. 2655, Seq. ID No. 2659, Seq. ID No. 2663, Seq. ID No. 2667, Seq. ID No. 2669, Seq. ID No. 2671, Seq. ID No. 2677, Seq. ID No. 2687, Seq. ID No. 2689, Seq. ID No. 2693, Seq. ID No. 2699, Seq. ID No. 2703, Seq. ID No. 2709, Seq. ID No. 2711, Seq. ID No. 2715, Seq. ID No. 2719, Seq. ID No. 2725, Seq. ID No. 2727, Seq. ID No. 2737, Seq. ID No. 2743, Seq. ID No. 2747, Seq. ID No. 2751, Seq. ID No. 2753, Seq. ID No. 2759, Seq. ID No. 2763, Seq. ID No. 2765, Seq. ID No. 2767, Seq. ID No. 2771, Seq. ID No. 2773, Seq. ID No. 2775, Seq. ID No. 2781, Seq. ID No. 2787, Seq. ID No. 2789, Seq. ID No. 2791, Seq. ID No. 2793, Seq. ID No. 2797, Seq. ID No. 2799, Seq. ID No. 2803, Seq. ID No. 2805, Seq. ID No. 2811, Seq. ID No. 2815, Seq. ID No. 2823, Seq. ID No. 2827, Seq. ID No. 2843, Seq. ID No. 2847, Seq. ID No. 2863, Seq. ID No. 2867, Seq. ID No. 2869, Seq. ID No. 2871, Seq. ID No. 2877, Seq. ID No. 2881, Seq. ID No. 2891, Seq. ID No. 2899, Seq. ID No. 2901, Seq. ID No. 2903, Seq. ID No. 2907, Seq. ID No. 2911, Seq. ID No. 2913, Seq. ID No. 2917, Seq. ID No. 2919, Seq. ID No. 2921, Seq. ID No. 2923, Seq. ID No. 2927, Seq. ID No. 2933, Seq. ID No. 2945, Seq. ID No. 2953, Seq. ID No. 2957, Seq. ID No. 2963, Seq. ID No. 2971, Seq. ID No. 2973, Seq. ID No. 2977, Seq. ID No. 2979, Seq. ID No. 2983, Seq. ID No. 2985, Seq. ID No. 2991, Seq. ID No. 2993, Seq. ID No. 2999, Seq. ID No. 3001, Seq. ID No. 3005, Seq. ID No. 3011, Seq. ID No. 3019, Seq. ID No. 3027, Seq. ID No. 3033, Seq. ID No. 3035, Seq. ID No. 3039, Seq. ID No. 3043, Seq. ID No. 3045, Seq. ID No. 3049, Seq. ID No. 3055, Seq. ID No. 3059, Seq. ID No. 3061, Seq. ID No. 3067, Seq. ID No. 3069, Seq. ID No. 3081, Seq. ID No. 3089, Seq. ID No. 3091, Seq. ID No. 3093, Seq. ID No. 3097, Seq. ID No. 3103, Seq. ID No. 3107, Seq. ID No. 3111, Seq. ID No. 3117, Seq. ID No. 3125, Seq. ID No. 3133, Seq. ID No. 3135, Seq. ID No. 3137, Seq. ID No. 3141, Seq. ID No. 3143, Seq. ID No. 3149, Seq. ID No. 3155, Seq. ID No. 3157, Seq. ID No. 3159, Seq. ID No. 3161, Seq. ID No. 3165, Seq. ID No. 3171, Seq. ID No. 3177, Seq. ID No. 3179, Seq. ID No. 3183, Seq. ID No. 3185, Seq. ID No. 3189, Seq. ID No. 3191, Seq. ID No. 3197, Seq. ID No. 3203, Seq. ID No. 3205, Seq. ID No. 3211, Seq. ID No. 3217; Seq. ID No. 3221, Seq. ID No. 3227, Seq. ID No. 3229, Seq. ID No. 3233, Seq. ID No. 3235, Seq. ID No. 3239, Seq. ID No. 3249, Seq. ID No. 3251, Seq. ID No. 3253, Seq. ID No. 3257, Seq. ID No. 3259, Seq. ID No. 3263, Seq. ID No. 3265, Seq. ID No. 3271, Seq. ID No. 3273, Seq. ID No. 3277, Seq. ID No. 3283, Seq. ID No. 3285, Seq. ID No. 3287, Seq. ID No. 3289, Seq. ID No. 3293, Seq. ID No. 3295, Seq. ID No. 3299, Seq. ID No. 3303, Seq. ID No. 3307, Seq. ID No. 3313, Seq. ID No. 3315, Seq. ID No. 3319, Seq. ID No. 3323, Seq. ID No. 3329, Seq. ID No. 3335, Seq. ID No. 3337, Seq. ID No. 3347, Seq. ID No. 3355, Seq. ID No. 3359, Seq. ID No. 3363, Seq. ID No. 3367, Seq. ID No. 3377, Seq. ID No. 3379, Seq. ID No. 3383, Seq. ID No. 3387, Seq. ID No. 3399, Seq. ID No. 3401, Seq. ID No. 3403, Seq. ID No. 3409, Seq. ID No. 3413, Seq. ID No. 3415, Seq. ID No. 3417, Seq. ID No. 3425, Seq. ID No. 3427, Seq. ID No. 3431, Seq. ID No. 3435, Seq. ID No. 3437, Seq. ID No. 3439, Seq. ID No. 3447, Seq. ID No. 3459, Seq. ID No. 3461, Seq. ID No. 3467, Seq. ID No. 3469, Seq. ID No. 3471, Seq. ID No. 3473, Seq. ID No. 3475, Seq. ID No. 3479, Seq. ID No. 3483, Seq. ID No. 3485, Seq. ID No. 3487, Seq. ID No. 3495, Seq. ID No. 3501, Seq. ID No. 3505, Seq. ID No. 3507, Seq. ID No. 3509, Seq. ID No. 3511, Seq. ID No. 3519, Seq. ID No. 3521, Seq. ID No. 3525, Seq. ID No. 3529, Seq. ID No. 3533, Seq. ID No. 3537, Seq. ID No. 3543, Seq. ID No. 3549, Seq. ID No. 3553, Seq. ID No. 3561, Seq. ID No. 3565, Seq. ID No. 3569, Seq. ID No. 3575, Seq. ID No. 3579, Seq. ID No. 3583, Seq. ID No. 3585, Seq. ID No. 3589, Seq. ID No. 3599, Seq. ID No. 3605, Seq. ID No. 3607, Seq. ID No. 3609, Seq. ID No. 3611, Seq. ID No. 3615, Seq. ID No. 3619, Seq. ID No. 3629, Seq. ID No. 3635, Seq. ID No. 3639, Seq. ID No. 3641, Seq. ID No. 3643, Seq. ID No. 3645, Seq. ID No. 3651, Seq. ID No. 3653, Seq. ID No. 3659, Seq. ID No. 3663, Seq. ID No. 3665, Seq. ID No. 3667, Seq. ID No. 3669, Seq. ID No. 3675, Seq. ID No. 3681, Seq. ID No. 3689, Seq. ID No. 3695, Seq. ID No. 3699, Seq. ID No. 3709, Seq. ID No. 3715, Seq. ID No. 3721, Seq. ID No. 3727, Seq. ID No. 3729, Seq. ID No. 3733, Seq. ID No. 3735, Seq. ID No. 3739, Seq. ID No. 3741, Seq. ID No. 3747, Seq. ID No. 3751, Seq. ID No. 3759, Seq. ID No. 3771, Seq. ID No. 3775, Seq. ID No. 3779, Seq. ID No. 3783, Seq. ID No. 3791, Seq. ID No. 3797, Seq. ID No. 3803, Seq. ID No. 3805, Seq. ID No. 3811, Seq. ID No. 3813, Seq. ID No. 3819, Seq. ID No. 3823, Seq. ID No. 3825, Seq. ID No. 3833, Seq. ID No. 3835, Seq. ID No. 3843, Seq. ID No. 3847, Seq. ID No. 3855, Seq. ID No. 3857, Seq. ID No. 3863, Seq. ID No. 3867, Seq. ID No. 3873, Seq. ID No. 3877, Seq. ID No. 3881, Seq. ID No. 3883, Seq. ID No. 3885, Seq. ID No. 3893, Seq. ID No. 3897, Seq. ID No. 3901, Seq. ID No. 3907, Seq. ID No. 3913, Seq. ID No. 3915, Seq. ID No. 3917, Seq. ID No. 3919, Seq. ID No. 3925, Seq. ID No. 3929, Seq. ID No. 3933, Seq. ID No. 3935, Seq. ID No. 3937, Seq. ID No. 3941, Seq. ID No. 3943, Seq. ID No. 3945, Seq. ID No. 3949, Seq. ID No. 3951, Seq. ID No. 3955, Seq. ID No. 3957, Seq. ID No. 3971, Seq. ID No. 3975, Seq. ID No. 3983, Seq. ID No. 3985, Seq. ID No. 3987, Seq. ID No. 3991, Seq. ID No. 4003, Seq. ID No. 4009, Seq. ID No. 4013, Seq. ID No. 4015, Seq. ID No. 4017, Seq. ID No. 4021, Seq. ID No. 4023, Seq. ID No. 4029, Seq. ID No. 4031, Seq. ID No. 4035, Seq. ID No. 4037, Seq. ID No. 4039, Seq. ID No. 4049, Seq. ID No. 4055, Seq. ID No. 4057, Seq. ID No. 4061, Seq. ID No. 4063, Seq. ID No. 4067, Seq. ID No. 4069, Seq. ID No. 4075, Seq. ID No. 4077, Seq. ID No. 4079, Seq. ID No. 4087, Seq. ID No. 4089, Seq. ID No. 4095, Seq. ID No. 4097, Seq. ID No. 4099, Seq. ID No. 4105, Seq. ID No. 4111, Seq. ID No. 4117, Seq. ID No. 4121, Seq. ID No. 4123, Seq. ID No. 4127, Seq. ID No. 4129, Seq. ID No. 4133, Seq. ID No. 4137, Seq. ID No. 4145, Seq. ID No. 4149, Seq. ID No. 4157, Seq. ID No. 4161, Seq. ID No. 4163, Seq. ID No. 4167, Seq. ID No. 4173, Seq. ID No. 4179, Seq. ID No. 4185, Seq. ID No. 4191, Seq. ID No. 4193, Seq. ID No. 4195, Seq. ID No. 4197, Seq. ID No. 4199, Seq. ID No. 4201, Seq. ID No. 4203, Seq. ID No. 4205, Seq. ID No. 4211, Seq. ID No. 4215, Seq. ID No. 4217, Seq. ID No. 4219, Seq. ID No. 4235, Seq. ID No. 4237, Seq. ID No. 4239, Seq. ID No. 4243, Seq. ID No. 4251, Seq. ID No. 4255, Seq. ID No. 4263, Seq. ID No. 4267, Seq. ID No. 4271, Seq. ID No. 4273, Seq. ID No. 4279, Seq. ID No. 4281, Seq. ID No. 4283, Seq. ID No. 4291, Seq. ID No. 4295, Seq. ID No. 4299, Seq. ID No. 4301, Seq. ID No. 4303, Seq. ID No. 4307, Seq. ID No. 4309, Seq. ID No. 4313, Seq. ID No. 4317, Seq. ID No. 4323, Seq. ID No. 4325, Seq. ID No. 4329, Seq. ID No. 4331, Seq. ID No. 4333, Seq. ID No. 4339, Seq. ID No. 4343, Seq. ID No. 4347, Seq. ID No. 4353, Seq. ID No. 4355, Seq. ID No. 4357, Seq. ID No. 4365, Seq. ID No. 4369, Seq. ID No. 4373, Seq. ID No. 4375, Seq. ID No. 4377, Seq. ID No. 4379, Seq. ID No. 4381, Seq. ID No. 4383, Seq. ID No. 4391, Seq. ID No. 4393, Seq. ID No. 4397, Seq. ID No. 4401, Seq. ID No. 4411, Seq. ID No. 4417, Seq. ID No. 4423, Seq. ID No. 4431, Seq. ID No. 4433, Seq. ID No. 4435, Seq. ID No. 4443, Seq. ID No. 4447, Seq. ID No. 4451, Seq. ID No. 4459, Seq. ID No. 4461, Seq. ID No. 4467, Seq. ID No. 4471, Seq. ID No. 4475, Seq. ID No. 4479, Seq. ID No. 4481, Seq. ID No. 4487, Seq. ID No. 4491, Seq. ID No. 4501, Seq. ID No. 4505, Seq. ID No. 4511, Seq. ID No. 4523, Seq. ID No. 4531, Seq. ID No. 4533, Seq. ID No. 4537, Seq. ID No. 4539, Seq. ID No. 4541, Seq. ID No. 4543, Seq. ID No. 4547, Seq. ID No. 4549, Seq. ID No. 4559, Seq. ID No. 4565, Seq. ID No. 4567, Seq. ID No. 4571, Seq. ID No. 4575, Seq. ID No. 4577, Seq. ID No. 4581, Seq. ID No. 4585, Seq. ID No. 4591, Seq. ID No. 4595, Seq. ID No. 4597, Seq. ID No. 4603, Seq. ID No. 4607, Seq. ID No. 4609, Seq. ID No. 4611, Seq. ID No. 4615, Seq. ID No. 4623, Seq. ID No. 4627, Seq. ID No. 4631, Seq. ID No. 4635, Seq. ID No. 4637, Seq. ID No. 4643, Seq. ID No. 4649, Seq. ID No. 4651, Seq. ID No. 4653, Seq. ID No. 4657, Seq. ID No. 4661, Seq. ID No. 4663, Seq. ID No. 4669, Seq. ID No. 4671, Seq. ID No. 4673, Seq. ID No. 4679, Seq. ID No. 4685, Seq. ID No. 4693, Seq. ID No. 4697, Seq. ID No. 4703, Seq. ID No. 4705, Seq. ID No. 4713, Seq. ID No. 4717, Seq. ID No. 4719, Seq. ID No. 4725, Seq. ID No. 4729, Seq. ID No. 4731, Seq. ID No. 4733, Seq. ID No. 4739, Seq. ID No. 4743, Seq. ID No. 4745, Seq. ID No. 4747, Seq. ID No. 4749, Seq. ID No. 4751, Seq. ID No. 4757, Seq. ID No. 4765, Seq. ID No. 4769, Seq. ID No. 4777, Seq. ID No. 4779, Seq. ID No. 4781, Seq. ID No. 4787, Seq. ID No. 4789, Seq. ID No. 4791, Seq. ID No. 4797, Seq. ID No. 4803, Seq. ID No. 4807, Seq. ID No. 4809, Seq. ID No. 4811, Seq. ID No. 4813, Seq. ID No. 4815, Seq. ID No. 4821, Seq. ID No. 4823, Seq. ID No. 4825, Seq. ID No. 4827, Seq. ID No. 4833, Seq. ID No. 4835, Seq. ID No. 4837, Seq. ID No. 4843, Seq. ID No. 4845, Seq. ID No. 4847, Seq. ID No. 4849, Seq. ID No. 4859, Seq. ID No. 4861, Seq. ID No. 4865, Seq. ID No. 4867, Seq. ID No. 4875, Seq. ID No. 4877, Seq. ID No. 4879, Seq. ID No. 4881, Seq. ID No. 4883, Seq. ID No. 4885, Seq. ID No. 4887, Seq. ID No. 4893, Seq. ID No. 4899, Seq. ID No. 4901, Seq. ID No. 4903, Seq. ID No. 4905, Seq. ID No. 4907, Seq. ID No. 4911, Seq. ID No. 4915, Seq. ID No. 4917, Seq. ID No. 4919, Seq. ID No. 4921, Seq. ID No. 4927, Seq. ID No. 4931, Seq. ID No. 4935, Seq. ID No. 4939, Seq. ID No. 4941, Seq. ID No. 4943, Seq. ID No. 4945, Seq. ID No. 4947, Seq. ID No. 4953, Seq. ID No. 4957, Seq. ID No. 4959, Seq. ID No. 4963, Seq. ID No. 4965, Seq. ID No. 4969, Seq. ID No. 4971, Seq. ID No. 4973, Seq. ID No. 4979, Seq. ID No. 4985, Seq. ID No. 4989, Seq. ID No. 4991, Seq. ID No. 4995, Seq. ID No. 4997, Seq. ID No. 5001, Seq. ID No. 5007, Seq. ID No. 5009, Seq. ID No. 5017, Seq. ID No. 5021, Seq. ID No. 5027, Seq. ID No. 5029, Seq. ID No. 5039, Seq. ID No. 5047, Seq. ID No. 5051, Seq. ID No. 5053, Seq. ID No. 5055, Seq. ID No. 5059, Seq. ID No. 5061, Seq. ID No. 5069, Seq. ID No. 5077, Seq. ID No. 5079, Seq. ID No. 5081, Seq. ID No. 5083, Seq. ID No. 5087, Seq. ID No. 5091, Seq. ID No. 5093, Seq. ID No. 5097, Seq. ID No. 5103, Seq. ID No. 5105, Seq. ID No. 5109, Seq. ID No. 5111, Seq. ID No. 5113, Seq. ID No. 5115, Seq. ID No. 5117, Seq. ID No. 5127, Seq. ID No. 5141, Seq. ID No. 5145, Seq. ID No. 5149, Seq. ID No. 5153, Seq. ID No. 5157, Seq. ID No. 5165, Seq. ID No. 5169, Seq. ID No. 5171, Seq. ID No. 5177, Seq. ID No. 5183, Seq. ID No. 5185, Seq. ID No. 5187, Seq. ID No. 5193, Seq. ID No. 5195, Seq. ID No. 5201, Seq. ID No. 5203, Seq. ID No. 5205, Seq. ID No. 5209, Seq. ID No. 5211, Seq. ID No. 5215, Seq. ID No. 5219, Seq. ID No. 5227, Seq. ID No. 5231, Seq. ID No. 5235, Seq. ID No. 5237, Seq. ID No. 5239, Seq. ID No. 5243, Seq. ID No. 5245, Seq. ID No. 5247, Seq. ID No. 5253, Seq. ID No. 5255, Seq. ID No. 5257, Seq. ID No. 5261, Seq. ID No. 5263, Seq. ID No. 5271, Seq. ID No. 5275, Seq. ID No. 5283, Seq. ID No. 5291, Seq. ID No. 5299, Seq. ID No. 5305, Seq. ID No. 5309, Seq. ID No. 5311, Seq. ID No. 5313, Seq. ID No. 5319, Seq. ID No. 5323, Seq. ID No. 5325, Seq. ID No. 5335, Seq. ID No. 5337, Seq. ID No. 5341, Seq. ID No. 5345, Seq. ID No. 5351, Seq. ID No. 5355, Seq. ID No. 5363, Seq. ID No. 5365, Seq. ID No. 5367, Seq. ID No. 5371, Seq. ID No. 5373, Seq. ID No. 5381, Seq. ID No. 5383, Seq. ID No. 5387, Seq. ID No. 5389, Seq. ID No. 5391, Seq. ID No. 5393, Seq. ID No. 5395, Seq. ID No. 5397, Seq. ID No. 5399, Seq. ID No. 5401, Seq. ID No. 5403, Seq. ID No. 5409, Seq. ID No. 5411, Seq. ID No. 5415, Seq. ID No. 5417, Seq. ID No. 5419, Seq. ID No. 5421, Seq. ID No. 5423, Seq. ID No. 5427, Seq. ID No. 5431, Seq. ID No. 5433, Seq. ID No. 5439, Seq. ID No. 5441, Seq. ID No. 5443, Seq. ID No. 5447, Seq. ID No. 5449, Seq. ID No. 5451, Seq. ID No. 5453, Seq. ID No. 5455, Seq. ID No. 5457, Seq. ID No. 5473, Seq. ID No. 5477, Seq. ID No. 5483, Seq. ID No. 5487, Seq. ID No. 5491, Seq. ID No. 5495, Seq. ID No. 5499, Seq. ID No. 5503, Seq. ID No. 5507, Seq. ID No. 5509, Seq. ID No. 5513, Seq. ID No. 5527, Seq. ID No. 5531, Seq. ID No. 5537, Seq. ID No. 5539, Seq. ID No. 5541, Seq. ID No. 5547, Seq. ID No. 5553, Seq. ID No. 5557, Seq. ID No. 5559, Seq. ID No. 5563, Seq. ID No. 5567, Seq. ID No. 5571, Seq. ID No. 5575, Seq. ID No. 5581, Seq. ID No. 5585, Seq. ID No. 5591, Seq. ID No. 5599, Seq. ID No. 5601, Seq. ID No. 5605, Seq. ID No. 5615, Seq. ID No. 5621, Seq. ID No. 5629, Seq. ID No. 5631, Seq. ID No. 5637, Seq. ID No. 5647, Seq. ID No. 5651, Seq. ID No. 5655, Seq. ID No. 5657, Seq. ID No. 5661, Seq. ID No. 5665, Seq. ID No. 5669, Seq. ID No. 5671, Seq. ID No. 5673, Seq. ID No. 5679, Seq. ID No. 5685, Seq. ID No. 5693, Seq. ID No. 5697, Seq. ID No. 5699, Seq. ID No. 5701, Seq. ID No. 5707, Seq. ID No. 5715, Seq. ID No. 5717, Seq. ID No. 5723, Seq. ID No. 5727, Seq. ID No. 5735, Seq. ID No. 5739, Seq. ID No. 5749, Seq. ID No. 5751, Seq. ID No. 5755, Seq. ID No. 5761, Seq. ID No. 5771, Seq. ID No. 5775, Seq. ID No. 5777, Seq. ID No. 5779, Seq. ID No. 5783, Seq. ID No. 5789, Seq. ID No. 5795, Seq. ID No. 5801, Seq. ID No. 5803, Seq. ID No. 5809, Seq. ID No. 5815, Seq. ID No. 5819, Seq. ID No. 5825, Seq. ID No. 5833, Seq. ID No. 5835, Seq. ID No. 5841, Seq. ID No. 5845, Seq. ID No. 5847, Seq. ID No. 5849, Seq. ID No. 5851; Seq. ID No. 5853, Seq. ID No. 5855, Seq. ID No. 5859, Seq. ID No. 5865, Seq. ID No. 5871, Seq. ID No. 5875, Seq. ID No. 5877, Seq. ID No. 5881, Seq. ID No. 5887, Seq. ID No. 5891, Seq. ID No. 5895, Seq. ID No. 5897, Seq. ID No. 5901, Seq. ID No. 5903, Seq. ID No. 5911, Seq. ID No. 5913, Seq. ID No. 5915, Seq. ID No. 5917, Seq. ID No. 5919, Seq. ID No. 5929, Seq. ID No. 5931, Seq. ID No. 5933, Seq. ID No. 5937, Seq. ID No. 5941, Seq. ID No. 5943, Seq. ID No. 5947, Seq. ID No. 5951, Seq. ID No. 5953, Seq. ID No. 5955, Seq. ID No. 5957, Seq. ID No. 5961, Seq. ID No. 5969, Seq. ID No. 5973, Seq. ID No. 5979, Seq. ID No. 5981, Seq. ID No. 5985, Seq. ID No. 5989, Seq. ID No. 5993, Seq. ID No. 6001, Seq. ID No. 6003, Seq. ID No. 6011, Seq. ID No. 6015, Seq. ID No. 6017, Seq. ID No. 6021, Seq. ID No. 6027, Seq. ID No. 6029, Seq. ID No. 6033, Seq. ID No. 6035, Seq. ID No. 6041, Seq. ID No. 6043, Seq. ID No. 6053, Seq. ID No. 6057, Seq. ID No. 6065, Seq. ID No. 6071, Seq. ID No. 6079, Seq. ID No. 6085, Seq. ID No. 6089, Seq. ID No. 6095, Seq. ID No. 6097, Seq. ID No. 6101, Seq. ID No. 6103, Seq. ID No. 6111, Seq. ID No. 6113, Seq. ID No. 6117, Seq. ID No. 6123, Seq. ID No. 6125, Seq. ID No. 6129, Seq. ID No. 6131, Seq. ID No. 6133, Seq. ID No. 6141, Seq. ID No. 6145, Seq. ID No. 6149, Seq. ID No. 6157, Seq. ID No. 6165, Seq. ID No. 6169, Seq. ID No. 6171, Seq. ID No. 6175, Seq. ID No. 6177, Seq. ID No. 6181, Seq. ID No. 6187, Seq. ID No. 6195, Seq. ID No. 6201, Seq. ID No. 6209, Seq. ID No. 6213, Seq. ID No. 6215, Seq. ID No. 6219, Seq. ID No. 6233, Seq. ID No. 6235, Seq. ID No. 6243, Seq. ID No. 6249, Seq. ID No. 6251, Seq. ID No. 6257, Seq. ID No. 6261, Seq. ID No. 6263, Seq. ID No. 6271, Seq. ID No. 6275, Seq. ID No. 6277, Seq. ID No. 6281, Seq. ID No. 6287, Seq. ID No. 6297, Seq. ID No. 6299, Seq. ID No. 6305, Seq. ID No. 6311, Seq. ID No. 6315, Seq. ID No. 6321, Seq. ID No. 6323, Seq. ID No. 6327, Seq. ID No. 6331, Seq. ID No. 6333, Seq. ID No. 6337, Seq. ID No. 6339, Seq. ID No. 6341, Seq. ID No. 6343, Seq. ID No. 6345, Seq. ID No. 6353, Seq. ID No. 6357, Seq. ID No. 6359, Seq. ID No. 6361, Seq. ID No. 6367, Seq. ID No. 6369, Seq. ID No. 6373, Seq. ID No. 6375, Seq. ID No. 6377, Seq. ID No. 6379, Seq. ID No. 6389, Seq. ID No. 6395, Seq. ID No. 6399, Seq. ID No. 6403, Seq. ID No. 6407, Seq. ID No. 6411, Seq. ID No. 6413, Seq. ID No. 6417, Seq. ID No. 6423, Seq. ID No. 6433, Seq. ID No. 6437, Seq. ID No. 6439, Seq. ID No. 6445, Seq. ID No. 6449, Seq. ID No. 6451, Seq. ID No. 6457, Seq. ID No. 6459, Seq. ID No. 6461, Seq. ID No. 6463, Seq. ID No. 6477, Seq. ID No. 6481, Seq. ID No. 6483, Seq. ID No. 6487, Seq. ID No. 6489, Seq. ID No. 6491, Seq. ID No. 6493, Seq. ID No. 6495, Seq. ID No. 6503, Seq. ID No. 6505, Seq. ID No. 6511, Seq. ID No. 6515, Seq. ID No. 6519, Seq. ID No. 6523, Seq. ID No. 6525, Seq. ID No. 6529, Seq. ID No. 6531, Seq. ID No. 6533, Seq. ID No. 6537, Seq. ID No. 6545, Seq. ID No. 6551, Seq. ID No. 6553, Seq. ID No. 6559, Seq. ID No. 6563, Seq. ID No. 6567, Seq. ID No. 6577, Seq. ID No. 6579, Seq. ID No. 6583, Seq. ID No. 6585, Seq. ID No. 6587, Seq. ID No. 6589, Seq. ID No. 6603, Seq. ID No. 6605, Seq. ID No. 6609, Seq. ID No. 6613, Seq. ID No. 6615, Seq. ID No. 6617, Seq. ID No. 6621, Seq. ID No. 6623, Seq. ID No. 6627, Seq. ID No. 6629, Seq. ID No. 6633, and Seq. ID No. 6639.

28. The polynucleotide of claim 7, wherein the polynucleotide encoded by the polynucleotide is identified by GeneMark™ ORF finder program, and the polynucleotide is selected from one of Seq. ID No. 1, Seq. ID No. 3, Seq. ID No. 11, Seq. ID No. 13, Seq. ID No. 15, Seq. ID No. 17, Seq. ID No. 25, Seq. ID No. 27, Seq. ID No. 29, Seq. ID No. 35, Seq. ID No. 37, Seq. ID No. 39, Seq. ID No. 41, Seq. ID No. 43, Seq. ID No. 45, Seq. ID No. 49, Seq. ID No. 53, Seq. ID No. 57, Seq. ID No. 61, Seq. ID No. 69, Seq. ID No. 71, Seq. ID No. 75, Seq. ID No. 81, Seq. ID No. 83, Seq. ID No. 89, Seq. ID No. 95, Seq. ID No. 101, Seq. ID No. 111, Seq. ID No. 119, Seq. ID No. 125, Seq. ID No. 129, Seq. ID No. 135, Seq. ID No. 137, Seq. ID No. 141, Seq. ID No. 147, Seq. ID No. 151, Seq. ID No. 155, Seq. ID No. 167, Seq. ID No. 171, Seq. ID No. 181, Seq. ID No. 185, Seq. ID No. 187, Seq. ID No. 189, Seq. ID No. 191, Seq. ID No. 195, Seq. ID No. 199, Seq. ID No. 207, Seq. ID No. 213, Seq. ID No. 219, Seq. ID No. 221, Seq. ID No. 225, Seq. ID No. 227, Seq. ID No. 229, Seq. ID No. 233, Seq. ID No. 239, Seq. ID No. 241, Seq. ID No. 243, Seq. ID No. 253, Seq. ID No. 261, Seq. ID No. 263, Seq. ID No. 267, Seq. ID No. 273, Seq. ID No. 275, Seq. ID No. 277, Seq. ID No. 279, Seq. ID No. 291, Seq. ID No. 293, Seq. ID No. 295, Seq. ID No. 299, Seq. ID No. 303, Seq. ID No. 309, Seq. ID No. 311, Seq. ID No. 315, Seq. ID No. 319, Seq. ID No. 323, Seq. ID No. 331, Seq. ID No. 341, Seq. ID No. 345, Seq. ID No. 351, Seq. ID No. 355, Seq. ID No. 361, Seq. ID No. 363, Seq. ID No. 365, Seq. ID No. 375, Seq. ID No. 379, Seq. ID No. 381, Seq. ID No. 383, Seq. ID No. 389, Seq. ID No. 391, Seq. ID No. 395, Seq. ID No. 399, Seq. ID No. 405, Seq. ID No. 409, Seq. ID No. 413, Seq. ID No. 417, Seq. ID No. 421, Seq. ID No. 423, Seq. ID No. 425, Seq. ID No. 427, Seq. ID No. 429, Seq. ID No. 435, Seq. ID No. 437, Seq. ID No. 439, Seq. ID No. 441, Seq. ID No. 443, Seq. ID No. 445, Seq. ID No. 447, Seq. ID No. 449, Seq. ID No. 459, Seq. ID No. 465, Seq. ID No. 485, Seq. ID No. 501, Seq. ID No. 505, Seq. ID No. 511, Seq. ID No. 513, Seq. ID No. 517, Seq. ID No. 519, Seq. ID No. 523, Seq. ID No. 529, Seq. ID No. 533, Seq. ID No. 535, Seq. ID No. 541, Seq. ID No. 543, Seq. ID No. 551, Seq. ID No. 553, Seq. ID No. 559, Seq. ID No. 563, Seq. ID No. 569, Seq. ID No. 575, Seq. ID No. 579, Seq. ID No. 583, Seq. ID No. 591, Seq. ID No. 595, Seq. ID No. 601, Seq. ID No. 605, Seq. ID No. 607, Seq. ID No. 611, Seq. ID No. 613, Seq. ID No. 615, Seq. ID No. 621, Seq. ID No. 627, Seq. ID No. 629, Seq. ID No. 633, Seq. ID No. 635, Seq. ID No. 637, Seq. ID No. 643, Seq. ID No. 649, Seq. ID No. 651, Seq. ID No. 655, Seq. ID No. 657, Seq. ID No. 669, Seq. ID No. 671, Seq. ID No. 675, Seq. ID No. 681, Seq. ID No. 683, Seq. ID No. 685, Seq. ID No. 693, Seq. ID No. 703, Seq. ID No. 705, Seq. ID No. 711, Seq. ID No. 725, Seq. ID No. 731, Seq. ID No. 735, Seq. ID No. 737, Seq. ID No. 741, Seq. ID No. 743, Seq. ID No. 749, Seq. ID No. 753, Seq. ID No. 759, Seq. ID No. 773, Seq. ID No. 781, Seq. ID No. 783, Seq. ID No. 785, Seq. ID No. 789, Seq. ID No. 793, Seq. ID No. 797, Seq. ID No. 803, Seq. ID No. 815, Seq. ID No. 819, Seq. ID No. 821, Seq. ID No. 827, Seq. ID No. 831, Seq. ID No. 833, Seq. ID No. 835, Seq. ID No. 837, Seq. ID No. 841, Seq. ID No. 843, Seq. ID No. 849, Seq. ID No. 851, Seq. ID No. 853, Seq. ID No. 857, Seq. ID No. 865, Seq. ID No. 869, Seq. ID No. 873, Seq. ID No. 877, Seq. ID No. 883, Seq. ID No. 887, Seq. ID No. 891, Seq. ID No. 895, Seq. ID No. 901, Seq. ID No. 907, Seq. ID No. 911, Seq. ID No. 917, Seq. ID No. 921, Seq. ID No. 927, Seq. ID No. 935, Seq. ID No. 939, Seq. ID No. 945, Seq. ID No. 951, Seq. ID No. 953, Seq. ID No. 959, Seq. ID No. 963, Seq. ID No. 967, Seq. ID No. 969, Seq. ID No. 977, Seq. ID No. 989, Seq. ID No. 995, Seq. ID No. 999, Seq. ID No. 1005, Seq. ID No. 1007, Seq. ID No. 1015, Seq. ID No. 1021, Seq. ID No. 1035, Seq. ID No. 1041, Seq. ID No. 1043, Seq. ID No. 1049, Seq. ID No. 1053, Seq. ID No. 1057, Seq. ID No. 1059, Seq. ID No. 1063, Seq. ID No. 1067, Seq. ID No. 1069, Seq. ID No. 1071, Seq. ID No. 1075, Seq. ID No. 1083, Seq. ID No. 1085, Seq. ID No. 1089, Seq. ID No. 1093, Seq. ID No. 1097, Seq. ID No. 1099, Seq. ID No. 1101, Seq. ID No. 1109, Seq. ID No. 1113, Seq. ID No. 1117, Seq. ID No. 1121, Seq. ID No. 1129, Seq. ID No. 1133, Seq. ID No. 1137, Seq. ID No. 1141, Seq. ID No. 1145, Seq. ID No. 1149, Seq. ID No. 1155, Seq. ID No. 1157, Seq. ID No. 1159, Seq. ID No. 1163, Seq. ID No. 1171, Seq. ID No. 1173, Seq. ID No. 1175, Seq. ID No. 1181, Seq. ID No. 1185, Seq. ID No. 1189, Seq. ID No. 1199, Seq. ID No. 1205, Seq. ID No. 1209, Seq. ID No. 1215, Seq. ID No. 1219, Seq. ID No. 1223, Seq. ID No. 1225, Seq. ID No. 1233, Seq. ID No. 1235, Seq. ID No. 1241, Seq. ID No. 1251, Seq. ID No. 1255, Seq. ID No. 1261, Seq. ID No. 1271, Seq. ID No. 1281, Seq. ID No. 1315, Seq. ID No. 1323, Seq. ID No. 1331, Seq. ID No. 1333, Seq. ID No. 1339, Seq. ID No. 1345, Seq. ID No. 1347, Seq. ID No. 1355, Seq. ID No. 1357, Seq. ID No. 1369, Seq. ID No. 1373, Seq. ID No. 1387, Seq. ID No. 1391, Seq. ID No. 1395, Seq. ID No. 1397, Seq. ID No. 1403, Seq. ID No. 1405, Seq. ID No. 1407, Seq. ID No. 1413, Seq. ID No. 1415, Seq. ID No. 1417, Seq. ID No. 1425, Seq. ID No. 1427, Seq. ID No. 1429, Seq. ID No. 1431, Seq. ID No. 1433, Seq. ID No. 1435, Seq. ID No. 1439, Seq. ID No. 1441, Seq. ID No. 1451, Seq. ID No. 1453, Seq. ID No. 1459, Seq. ID No. 1461, Seq. ID No. 1469, Seq. ID No. 1471, Seq. ID No. 1473, Seq. ID No. 1475, Seq. ID No. 1477, Seq. ID No. 1483, Seq. ID No. 1487, Seq. ID No. 1489, Seq. ID No. 1493, Seq. ID No. 1495, Seq. ID No. 1497, Seq. ID No. 1501, Seq. ID No. 1503, Seq. ID No. 1507, Seq. ID No. 1509, Seq. ID No. 1517, Seq. ID No. 1521, Seq. ID No. 1527, Seq. ID No. 1531, Seq. ID No. 1537, Seq. ID No. 1541, Seq. ID No. 1543, Seq. ID No. 1549, Seq. ID No. 1561, Seq. ID No. 1563, Seq. ID No. 1565, Seq. ID No. 1569, Seq. ID No. 1573, Seq. ID No. 1575, Seq. ID No. 1581, Seq. ID No. 1587, Seq. ID No. 1591, Seq. ID No. 1595, Seq. ID No. 1597, Seq. ID No. 1605, Seq. ID No. 1609, Seq. ID No. 1613, Seq. ID No. 1619, Seq. ID No. 1623, Seq. ID No. 1627, Seq. ID No. 1629, Seq. ID No. 1635, Seq. ID No. 1641, Seq. ID No. 1647, Seq. ID No. 1651, Seq. ID No. 1657, Seq. ID No. 1659, Seq. ID No. 1667, Seq. ID No. 1669, Seq. ID No. 1679, Seq. ID No. 1683, Seq. ID No. 1687, Seq. ID No. 1691, Seq. ID No. 1697, Seq. ID No. 1699, Seq. ID No. 1701, Seq. ID No. 1707, Seq. ID No. 1709, Seq. ID No. 1713, Seq. ID No. 1717, Seq. ID No. 1721, Seq. ID No. 1725, Seq. ID No. 1735, Seq. ID No. 1737, Seq. ID No. 1739, Seq. ID No. 1743, Seq. ID No. 1745, Seq. ID No. 1749, Seq. ID No. 1755, Seq. ID No. 1769, Seq. ID No. 1773, Seq. ID No. 1777, Seq. ID No. 1785, Seq. ID No. 1789, Seq. ID No. 1791, Seq. ID No. 1797, Seq. ID No. 1799, Seq. ID No. 1803, Seq. ID No. 1805, Seq. ID No. 1807, Seq. ID No. 1817, Seq. ID No. 1819, Seq. ID No. 1821, Seq. ID No. 1825, Seq. ID No. 1827, Seq. ID No. 1831, Seq. ID No. 1835, Seq. ID No. 1841, Seq. ID No. 1845, Seq. ID No. 1847, Seq. ID No. 1849, Seq. ID No. 1851, Seq. ID No. 1855, Seq. ID No. 1859, Seq. ID No. 1861, Seq. ID No. 1865, Seq. ID No. 1867, Seq. ID No. 1871, Seq. ID No. 1873, Seq. ID No. 1877, Seq. ID No. 1879, Seq. ID No. 1887, Seq. ID No. 1889, Seq. ID No. 1895, Seq. ID No. 1897, Seq. ID No. 1899, Seq. ID No. 1901, Seq. ID No. 1905, Seq. ID No. 1909, Seq. ID No. 1917, Seq. ID No. 1921, Seq. ID No. 1925, Seq. ID No. 1931, Seq. ID No. 1933, Seq. ID No. 1937, Seq. ID No. 1939, Seq. ID No. 1945, Seq. ID No. 1947, Seq. ID No. 1951, Seq. ID No. 1955, Seq. ID No. 1959, Seq. ID No. 1961, Seq. ID No. 1965, Seq. ID No. 1967, Seq. ID No. 1969, Seq. ID No. 1973, Seq. ID No. 1977, Seq. ID No. 1979, Seq. ID No. 1983, Seq. ID No. 1989, Seq. ID No. 1991, Seq. ID No. 1997, Seq. ID No. 2001, Seq. ID No. 2003, Seq. ID No. 2011, Seq. ID No. 2021, Seq. ID No. 2023, Seq. ID No. 2029, Seq. ID No. 2033, Seq. ID No. 2037, Seq. ID No. 2039, Seq. ID No. 2045, Seq. ID No. 2047, Seq. ID No. 2051, Seq. ID No. 2057, Seq. ID No. 2061, Seq. ID No. 2067, Seq. ID No. 2069, Seq. ID No. 2071, Seq. ID No. 2073, Seq. ID No. 2077, Seq. ID No. 2089, Seq. ID No. 2093, Seq. ID No. 2095, Seq. ID No. 2099, Seq. ID No. 2101, Seq. ID No. 2113, Seq. ID No. 2117, Seq. ID No. 2123, Seq. ID No. 2135, Seq. ID No. 2137, Seq. ID No. 2139, Seq. ID No. 2143, Seq. ID No. 2147, Seq. ID No. 2153, Seq. ID No. 2159, Seq. ID No. 2163, Seq. ID No. 2167, Seq. ID No. 2175, Seq. ID No. 2181, Seq. ID No. 2185, Seq. ID No. 2189, Seq. ID No. 2193, Seq. ID No. 2195, Seq. ID No. 2197, Seq. ID No. 2205, Seq. ID No. 2217, Seq. ID No. 2219, Seq. ID No. 2223, Seq. ID No. 2225, Seq. ID No. 2229, Seq. ID No. 2235, Seq. ID No. 2239, Seq. ID No. 2243, Seq. ID No. 2245, Seq. ID No. 2247, Seq. ID No. 2253, Seq. ID No. 2255, Seq. ID No. 2257, Seq. ID No. 2259, Seq. ID No. 2261, Seq. ID No. 2265, Seq. ID No. 2269, Seq. ID No. 2273, Seq. ID No. 2285, Seq. ID No. 2297, Seq. ID No. 2299, Seq. ID No. 2301, Seq. ID No. 2313, Seq. ID No. 2327, Seq. ID No. 2331, Seq. ID No. 2335, Seq. ID No. 2337, Seq. ID No. 2341, Seq. ID No. 2343, Seq. ID No. 2345, Seq. ID No. 2349, Seq. ID No. 2351, Seq. ID No. 2357, Seq. ID No. 2359, Seq. ID No. 2361, Seq. ID No. 2365, Seq. ID No. 2369, Seq. ID No. 2373, Seq. ID No. 2377, Seq. ID No. 2383, Seq. ID No. 2397, Seq. ID No. 2401, Seq. ID No. 2403, Seq. ID No. 2409, Seq. ID No. 2411, Seq. ID No. 2417, Seq. ID No. 2421, Seq. ID No. 2423, Seq. ID No. 2425, Seq. ID No. 2429, Seq. ID No. 2435, Seq. ID No. 2437, Seq. ID No. 2439, Seq. ID No. 2441, Seq. ID No. 2445, Seq. ID No. 2449, Seq. ID No. 2451, Seq. ID No. 2453, Seq. ID No. 2461, Seq. ID No. 2465, Seq. ID No. 2467, Seq. ID No. 2471, Seq. ID No. 2473, Seq. ID No. 2477, Seq. ID No. 2479, Seq. ID No. 2481, Seq. ID No. 2485, Seq. ID No. 2487, Seq. ID No. 2495, Seq. ID No. 2505, Seq. ID No. 2507, Seq. ID No. 2509, Seq. ID. No. 2519, Seq. ID No. 2525, Seq. ID No. 2527, Seq. ID No. 2529, Seq. ID No. 2535, Seq. ID No. 2537, Seq. ID No. 2539, Seq. ID No. 2547, Seq. ID No. 2551, Seq. ID No. 2563, Seq. ID No. 2565, Seq. ID No. 2573, Seq. ID No. 2577, Seq. ID No. 2579, Seq. ID No. 2585, Seq. ID No. 2593, Seq. ID No. 2595, Seq. ID No. 2599, Seq. ID No. 2607, Seq. ID No. 2609, Seq. ID No. 2613, Seq. ID No. 2615, Seq. ID No. 2619, Seq. ID No. 2623, Seq. ID No. 2625, Seq. ID No. 2631, Seq. ID No. 2633, Seq. ID No. 2639, Seq. ID No. 2641, Seq. ID No. 2643, Seq. ID No. 2647, Seq. ID No. 2651, Seq. ID No. 2655, Seq. ID No. 2657, Seq. ID No. 2661, Seq. ID No. 2665, Seq. ID No. 2669, Seq. ID No. 2671, Seq. ID No. 2673, Seq. ID No. 2681, Seq. ID No. 2689, Seq. ID No. 2693, Seq. ID No. 2699, Seq. ID No. 2703, Seq. ID No. 2709, Seq. ID No. 2711, Seq. ID No. 2713, Seq. ID No. 2719, Seq. ID No. 2721, Seq. ID No. 2727, Seq. ID No. 2737, Seq. ID No. 2741, Seq. ID No. 2745, Seq. ID No. 2749, Seq. ID No. 2753, Seq. ID No. 2757, Seq. ID No. 2761, Seq. ID No. 2765, Seq. ID No. 2767, Seq. ID No. 2769, Seq. ID No. 2773, Seq. ID No. 2775, Seq. ID No. 2777, Seq. ID No. 2787, Seq. ID No. 2789, Seq. ID No. 2791, Seq. ID No. 2793, Seq. ID No. 2795, Seq. ID No. 2799, Seq. ID No. 2803, Seq. ID No. 2815, Seq. ID No. 2817, Seq. ID No. 2819, Seq. ID No. 2825, Seq. ID No. 2843, Seq. ID No. 2847, Seq. ID No. 2849, Seq. ID No. 2851, Seq. ID No. 2857, Seq. ID No. 2859, Seq. ID No. 2863, Seq. ID No. 2867, Seq. ID No. 2869, Seq. ID No. 2871, Seq. ID No. 2877, Seq. ID No. 2879, Seq. ID No. 2889, Seq. ID No. 2891, Seq. ID No. 2897, Seq. ID No. 2901, Seq. ID No. 2903, Seq. ID No. 2907, Seq. ID No. 2909, Seq. ID No. 2913, Seq. ID No. 2915, Seq. ID No. 2919, Seq. ID No. 2921, Seq. ID No. 2923, Seq. ID No. 2925, Seq. ID No. 2929, Seq. ID No. 2935, Seq. ID No. 2943, Seq. ID No. 2951, Seq. ID No. 2955, Seq. ID No. 2963, Seq. ID No. 2965, Seq. ID No. 2971, Seq. ID No. 2973, Seq. ID No. 2975, Seq. ID No. 2979, Seq. ID No. 2981, Seq. ID No. 2985, Seq. ID No. 2989, Seq. ID No. 2993, Seq. ID No. 2999, Seq. ID No. 3001, Seq. ID No. 3003, Seq. ID No. 3017, Seq. ID No. 3027, Seq. ID No. 3033, Seq. ID No. 3035, Seq. ID No. 3039, Seq. ID No. 3041, Seq. ID No. 3043, Seq. ID No. 3045, Seq. ID No. 3047, Seq. ID No. 3055, Seq. ID No. 3057, Seq. ID No. 3061, Seq. ID No. 3065, Seq. ID No. 3069, Seq. ID No. 3079, Seq. ID No. 3089, Seq. ID No. 3091, Seq. ID No. 3093, Seq. ID No. 3095, Seq. ID No. 3103, Seq. ID No. 3105, Seq. ID No. 3109, Seq. ID No. 3113, Seq. ID No. 3125, Seq. ID No. 3127, Seq. ID No. 3135, Seq. ID No. 3137, Seq. ID No. 3141, Seq. ID No. 3143, Seq. ID No. 3147, Seq. ID No. 3155, Seq. ID No. 3157, Seq. ID No. 3159, Seq. ID No. 3161, Seq. ID No. 3165, Seq. ID No. 3169, Seq. ID No. 3175, Seq. ID No. 3179, Seq. ID No. 3183, Seq. ID No. 3185, Seq. ID No. 3189, Seq. ID No. 3191, Seq. ID No. 3195, Seq. ID No. 3199, Seq. ID No. 3205, Seq. ID No. 3211, Seq. ID No. 3217, Seq. ID No. 3221, Seq. ID No. 3225, Seq. ID No. 3229, Seq. ID No. 3231, Seq. ID No. 3237, Seq. ID No. 3239, Seq. ID No. 3247, Seq. ID No. 3251, Seq. ID No. 3253, Seq. ID No. 3255, Seq. ID No. 3259, Seq. ID No. 3261, Seq. ID No. 3267, Seq. ID No. 3273, Seq. ID No. 3277, Seq. ID No. 3279, Seq. ID No. 3285, Seq. ID No. 3287, Seq. ID No. 3289, Seq. ID No. 3293, Seq. ID No. 3295, Seq. ID No. 3299, Seq. ID No. 3301, Seq. ID No. 3305, Seq. ID No. 3309, Seq. ID No. 3315, Seq. ID No. 3319, Seq. ID No. 3321, Seq. ID No. 3327, Seq. ID No. 3333, Seq. ID No. 3337, Seq. ID No. 3343, Seq. ID No. 3353, Seq. ID No. 3359, Seq. ID No. 3361, Seq. ID No. 3365, Seq. ID No. 3373, Seq. ID No. 3381, Seq. ID No. 3385, Seq. ID No. 3393, Seq. ID No. 3401, Seq. ID No. 3407, Seq. ID No. 3411, Seq. ID No. 3415, Seq. ID No. 3417, Seq. ID No. 3423, Seq. ID No. 3429, Seq. ID No. 3437, Seq. ID No. 3439, Seq. ID No. 3443, Seq. ID No. 3459, Seq. ID No. 3461, Seq. ID No. 3465, Seq. ID No. 3469, Seq. ID No. 3471, Seq. ID No. 3473, Seq. ID No. 3475, Seq. ID No. 3477, Seq. ID No. 3483, Seq. ID No. 3487, Seq. ID No. 3495, Seq. ID No. 3497, Seq. ID No. 3505, Seq. ID No. 3507, Seq. ID No. 3509, Seq. ID No. 3511, Seq. ID No. 3515, Seq. ID No. 3521, Seq. ID No. 3523, Seq. ID No. 3529, Seq. ID No. 3535, Seq. ID No. 3541, Seq. ID No. 3551, Seq. ID No. 3559, Seq. ID No. 3563, Seq. ID No. 3567, Seq. ID No. 3575, Seq. ID No. 3577, Seq. ID No. 3583, Seq. ID No. 3585, Seq. ID No. 3589, Seq. ID No. 3591, Seq. ID No. 3599, Seq. ID No. 3601, Seq. ID No. 3607, Seq. ID No. 3609, Seq. ID No. 3611, Seq. ID No. 3615, Seq. ID No. 3619, Seq. ID No. 3629, Seq. ID No. 3631, Seq. ID No. 3633, Seq. ID No. 3637, Seq. ID No. 3641, Seq. ID No. 3643, Seq. ID No. 3645, Seq. ID No. 3651, Seq. ID No. 3653, Seq. ID No. 3655, Seq. ID No. 3661, Seq. ID No. 3665, Seq. ID No. 3667, Seq. ID No. 3669, Seq. ID No. 3675, Seq. ID No. 3681, Seq. ID No. 3689, Seq. ID No. 3695, Seq. ID No. 3697, Seq. ID No. 3705, Seq. ID No. 3711, Seq. ID No. 3719, Seq. ID No. 3725, Seq. ID No. 3729, Seq. ID No. 3731, Seq. ID No. 3735, Seq. ID No. 3739, Seq. ID No. 3741, Seq. ID No. 3743, Seq. ID No. 3749, Seq. ID No. 3759, Seq. ID No. 3769, Seq. ID No. 3773, Seq. ID No. 3777, Seq. ID No. 3781, Seq. ID No. 3787, Seq. ID No. 3795, Seq. ID No. 3801, Seq. ID No. 3805, Seq. ID No. 3809, Seq. ID No. 3813, Seq. ID No. 3819, Seq. ID No. 3821, Seq. ID No. 3825, Seq. ID No. 3827, Seq. ID No. 3829, Seq. ID No. 3831, Seq. ID No. 3835, Seq. ID No. 3841, Seq. ID No. 3845, Seq. ID No. 3855, Seq. ID No. 3857, Seq. ID No. 3863, Seq. ID No. 3867, Seq. ID No. 3869, Seq. ID No. 3875, Seq. ID No. 3881, Seq. ID No. 3883, Seq. ID No. 3885, Seq. ID No. 3887, Seq. ID No. 3,897, Seq. ID No. 3901, Seq. ID No. 3905, Seq. ID No. 3909, Seq. ID No. 3913, Seq. ID No. 3915, Seq. ID No. 3917, Seq. ID No. 3919, Seq. ID No. 3921, Seq. ID No. 3929, Seq. ID No. 3931, Seq. ID No. 3935, Seq. ID No. 3941, Seq. ID No. 3943, Seq. ID No. 3945, Seq. ID No. 3947, Seq. ID No. 3951, Seq. ID No. 3953, Seq. ID No. 3957, Seq. ID No. 3971, Seq. ID No. 3977, Seq. ID No. 3981, Seq. ID No. 3985, Seq. ID No. 3987, Seq. ID No. 3989, Seq. ID No. 3993, Seq. ID No. 4001, Seq. ID No. 4005, Seq. ID No. 4011, Seq. ID No. 4015, Seq. ID No. 4017, Seq. ID No. 4019, Seq. ID No. 4023, Seq. ID No. 4029, Seq. ID No. 4031, Seq. ID No. 4033, Seq. ID No. 4037, Seq. ID No. 4039, Seq. ID No. 4049, Seq. ID No. 4051, Seq. ID No. 4057, Seq. ID No. 4059, Seq. ID No. 4065, Seq. ID No. 4069, Seq. ID No. 4075, Seq. ID No. 4079, Seq. ID No. 4085, Seq. ID No. 4089, Seq. ID No. 4095, Seq. ID No. 4097, Seq. ID No. 4099, Seq. ID No. 4105, Seq. ID No. 4111, Seq. ID No. 4115, Seq. ID No. 4121, Seq. ID No. 4123, Seq. ID No. 4127, Seq. ID No. 4129, Seq. ID No. 4131, Seq. ID No. 4137, Seq. ID No. 4141, Seq. ID No. 4147, Seq. ID No. 4157, Seq. ID No. 4159, Seq. ID No. 4163, Seq. ID No. 4165, Seq. ID No. 4169, Seq. ID No. 4179, Seq. ID No. 4183, Seq. ID No. 4187, Seq. ID No. 4193, Seq. ID No. 4195, Seq. ID No. 4197, Seq. ID No. 4199, Seq. ID No. 4201, Seq. ID No. 4205, Seq. ID No. 4207, Seq. ID No. 4213, Seq. ID No. 4219, Seq. ID No. 4235, Seq. ID No. 4237, Seq. ID No. 4239, Seq. ID No. 4243, Seq. ID No. 4245, Seq. ID No. 4255, Seq. ID No. 4259, Seq. ID No. 4267, Seq. ID No. 4271, Seq. ID No. 4277, Seq. ID No. 4279, Seq. ID No. 4281, Seq. ID No. 4283, Seq. ID No. 4289, Seq. ID No. 4293, Seq. ID No. 4299, Seq. ID No. 4301, Seq. ID No. 4303, Seq. ID No. 4305, Seq. ID No. 4307, Seq. ID No. 4309, Seq. ID No. 4313, Seq. ID No. 4315, Seq. ID No. 4323, Seq. ID No. 4325, Seq. ID No. 4327, Seq. ID No. 4331, Seq. ID No. 4333, Seq. ID No. 4337, Seq. ID No. 4341, Seq. ID No. 4345, Seq. ID No. 4349, Seq. ID No. 4355, Seq. ID No. 4357, Seq. ID No. 4365, Seq. ID No. 4367, Seq. ID No. 4371, Seq. ID No. 4375, Seq. ID No. 4377, Seq. ID No. 4379, Seq. ID No. 4383, Seq. ID No. 4387, Seq. ID No. 4393, Seq. ID No. 4397, Seq. ID No. 4401, Seq. ID No. 4409, Seq. ID No. 4413, Seq. ID No. 4421, Seq. ID No. 4433, Seq. ID No. 4435, Seq. ID No. 4445, Seq. ID No. 4449, Seq. ID No. 4457, Seq. ID No. 4461, Seq. ID No. 4463, Seq. ID No. 4471, Seq. ID No. 4475, Seq. ID No. 4477, Seq. ID No. 4481, Seq. ID No. 4487, Seq. ID No. 4491, Seq. ID No. 4499, Seq. ID No. 4501, Seq. ID No. 4503, Seq. ID No. 4509, Seq. ID No. 4517, Seq. ID No. 4531, Seq. ID No. 4533, Seq. ID No. 4535, Seq. ID No. 4539, Seq. ID No. 4541, Seq. ID No. 4545, Seq. ID No. 4549, Seq. ID No. 4557, Seq. ID No. 4563, Seq. ID No. 4567, Seq. ID No. 4569, Seq. ID No. 4575, Seq. ID No. 4577, Seq. ID No. 4579, Seq. ID No. 4583, Seq. ID No. 4591, Seq. ID No. 4593, Seq. ID No. 4597, Seq. ID No. 4601, Seq. ID No. 4605, Seq. ID No. 4609, Seq. ID No. 4611, Seq. ID No. 4613, Seq. ID No. 4623, Seq. ID No. 4625, Seq. ID No. 4629, Seq. ID No. 4633, Seq. ID No. 4637, Seq. ID No. 4641, Seq. ID No. 4645,.Seq. ID No. 4651, Seq. ID No. 4653, Seq. ID No. 4655, Seq. ID No. 4661, Seq. ID No. 4663, Seq. ID No. 4669, Seq. ID No. 4671, Seq. ID No. 4673, Seq. ID No. 4675, Seq. ID No. 4685, Seq. ID No. 4689, Seq. ID No. 4697, Seq. ID No. 4703, Seq. ID No. 4705, Seq. ID No. 4713, Seq. ID No. 4717, Seq. ID No. 4719, Seq. ID No. 4723, Seq. ID No. 4729, Seq. ID No. 4731, Seq. ID No. 4733, Seq. ID No. 4739, Seq. ID No. 4743, Seq. ID No. 4745, Seq. ID No. 4747, Seq. ID No. 4749, Seq. ID No. 4751, Seq. ID No. 4753, Seq. ID No. 4761, Seq. ID No. 4767, Seq. ID No. 4771, Seq. ID No. 4779, Seq. ID No. 4781, Seq. ID No. 4783, Seq. ID No. 4785, Seq. ID No. 4789, Seq. ID No. 4791, Seq. ID No. 4793, Seq. ID No. 4799, Seq. ID No. 4807, Seq. ID No. 4809, Seq. ID No. 4811, Seq. ID No. 4813, Seq. ID No. 4815, Seq. ID No. 4821, Seq. ID No. 4825, Seq. ID No. 4827, Seq. ID No. 4829, Seq. ID No. 4837, Seq. ID No. 4839, Seq. ID No. 4845, Seq. ID No. 4847, Seq. ID No. 4849, Seq. ID No. 4857, Seq. ID No. 4861, Seq. ID No. 4865, Seq. ID No. 4867, Seq. ID No. 4875, Seq. ID No. 4879, Seq. ID No. 4881, Seq. ID No. 4883, Seq. ID No. 4885, Seq. ID No. 4887, Seq. ID No. 4893, Seq. ID No. 4899, Seq. ID No. 4901, Seq. ID No. 4903, Seq. ID No. 4905, Seq. ID No. 4907, Seq. ID No. 4909, Seq. ID No. 4915, Seq. ID No. 4917, Seq. ID No. 4919, Seq. ID No. 4921, Seq. ID No. 4925, Seq. ID No. 4929, Seq. ID No. 4933, Seq. ID No. 4937, Seq. ID No. 4941, Seq. ID No. 4943, Seq. ID No. 4945, Seq. ID No. 4947, Seq. ID No. 4953, Seq. ID No. 4955, Seq. ID No. 4959, Seq. ID No. 4961, Seq. ID No. 4965, Seq. ID No. 4967, Seq. ID No. 4971, Seq. ID No. 4973, Seq. ID No. 4977, Seq. ID No. 4985, Seq. ID No. 4989, Seq. ID No. 4991, Seq. ID No. 4995, Seq. ID No. 4997, Seq. ID No. 5005, Seq. ID No. 5009, Seq. ID No. 5015, Seq. ID No. 5019, Seq. ID No. 5025, Seq. ID No. 5029, Seq. ID No. 5037, Seq. ID No. 5045, Seq. ID No. 5049, Seq. ID No. 5053, Seq. ID No. 5055, Seq. ID No. 5059, Seq. ID No. 5061, Seq. ID No. 5065, Seq. ID No. 5071, Seq. ID No. 5073, Seq. ID No. 5079, Seq. ID No. 5081, Seq. ID No. 5083, Seq. ID No. 5087, Seq. ID No. 5089, Seq. ID No. 5093, Seq. ID No. 5095, Seq. ID No. 5103, Seq. ID No. 5105, Seq. ID No. 5107, Seq. ID No. 5111, Seq. ID No. 5113, Seq. ID No. 5115, Seq. ID No. 5117, Seq. ID No. 5119, Seq. ID No. 5141, Seq. ID No. 5143, Seq. ID No. 5147, Seq. ID No. 5153, Seq. ID No. 5155, Seq. ID No. 5163, Seq. ID No. 5169, Seq. ID No. 5171, Seq. ID No. 5173, Seq. ID No. 5179, Seq. ID-No. 5185, Seq. ID No. 5187, Seq. ID No. 5193, Seq. ID No. 5195, Seq. ID No. 5201, Seq. ID No. 5203, Seq. ID No. 5205, Seq. ID No. 5209, Seq. ID No. 5211, Seq. ID No. 5213, Seq. ID No. 5217, Seq. ID No. 5225, Seq. ID No. 5231, Seq. ID No. 5233, Seq. ID No. 5237, Seq. ID No. 5239, Seq. ID No. 5245, Seq. ID No. 5247, Seq. ID No. 5249, Seq. ID No. 5255, Seq. ID No. 5257, Seq. ID No. 5259, Seq. ID No. 5263, Seq. ID No. 5269, Seq. ID No. 5273, Seq. ID No. 5283, Seq. ID No. 5289, Seq. ID No. 5293, Seq. ID No. 5307, Seq. ID No. 5311, Seq. ID No. 5313, Seq. ID No. 5315, Seq. ID No. 5323, Seq. ID No. 5325, Seq. ID No. 5335, Seq. ID No. 5337, Seq. ID No. 5339, Seq. ID No. 5345, Seq. ID No. 5347, Seq. ID No. 5349, Seq. ID No. 5353, Seq. ID No. 5361, Seq. ID No. 5365, Seq. ID No. 5367, Seq. ID No. 5369, Seq. ID No. 5373, Seq. ID No. 5375, Seq. ID No. 5387, Seq. ID No. 5389, Seq. ID No. 5391, Seq. ID No. 5393, Seq. ID No. 5395, Seq. ID No. 5397, Seq. ID No. 5399, Seq. ID No. 5401, Seq. ID No. 5403, Seq. ID No. 5405, Seq. ID No. 5411, Seq. ID No. 5415, Seq. ID No. 5417, Seq. ID No. 5419, Seq. ID No. 5421, Seq. ID No. 5423, Seq. ID No. 5429, Seq. ID No. 5433, Seq. ID No. 5441, Seq. ID No. 5443, Seq. ID No. 5445, Seq. ID No. 5449, Seq. ID No. 5451, Seq. ID No. 5453, Seq. ID No. 5455, Seq. ID No. 5457, Seq. ID No. 5469, Seq. ID No. 5475, Seq. ID No. 5483, Seq. ID No. 5485, Seq. ID No. 5489, Seq. ID No. 5493, Seq. ID No. 5499, Seq. ID No. 5501, Seq. ID No. 5505, Seq. ID No. 5509, Seq. ID No. 5511, Seq. ID No. 5523, Seq. ID No. 5529, Seq. ID No. 5535, Seq. ID No. 5539, Seq. ID No. 5541, Seq. ID No. 5547, Seq. ID No. 5551, Seq. ID No. 5555, Seq. ID No. 5559, Seq. ID No. 5561, Seq. ID No. 5565, Seq. ID No. 5571, Seq. ID No. 5573, Seq. ID No. 5579, Seq. ID No. 5583, Seq. ID No. 5589, Seq. ID No. 5599, Seq. ID No. 5601, Seq. ID No. 5603, Seq. ID No. 5613, Seq. ID No. 5621, Seq. ID No. 5627, Seq. ID No. 5631, Seq. ID No. 5635, Seq. ID No. 5645, Seq. ID No. 5651, Seq. ID No. 5653, Seq. ID No. 5657, Seq. ID No. 5661, Seq. ID No. 5663, Seq. ID No. 5665, Seq. ID No. 5669, Seq. ID No. 5671, Seq. ID No. 5673, Seq. ID No. 5675, Seq. ID No. 5681, Seq. ID No. 5693, Seq. ID No. 5695, Seq. ID No. 5699, Seq. ID No. 5701, Seq. ID No. 5707, Seq. ID No. 5713, Seq. ID No. 5717, Seq. ID No. 5723, Seq. ID No. 5725, Seq. ID No. 5731, Seq. ID No. 5737, Seq. ID No. 5747, Seq. ID No. 5751, Seq. ID No. 5755, Seq. ID No. 5759, Seq. ID No. 5771, Seq. ID No. 5773, Seq. ID No. 5777, Seq. ID No. 5779, Seq. ID No. 5781, Seq. ID No. 5785, Seq. ID No. 5793, Seq. ID No. 5799, Seq. ID No. 5805, Seq. ID No. 5811, Seq. ID No. 5815, Seq. ID No. 5817, Seq. ID No. 5823, Seq. ID No. 5831, Seq. ID No. 5835, Seq. ID No. 5839, Seq. ID No. 5843, Seq. ID No. 5847, Seq. ID No. 5849, Seq. ID No. 5851, Seq. ID No. 5853, Seq. ID No. 5855, Seq. ID No. 5857, Seq. ID No. 5861, Seq. ID No. 5867, Seq. ID No. 5875, Seq. ID No. 5877, Seq. ID No. 5881, Seq. ID No. 5889, Seq. ID No. 5895, Seq. ID No. 5897, Seq. ID No. 5901, Seq. ID No. 5903, Seq. ID No. 5907, Seq. ID No. 5913, Seq. ID No. 5915, Seq. ID No. 5917, Seq. ID No. 5919, Seq. ID No. 5925, Seq. ID No. 5931, Seq. ID No. 5933, Seq. ID No. 5935, Seq. ID No. 5941, Seq. ID No. 5943, Seq. ID No. 5945, Seq. ID No. 5949, Seq. ID No. 5955, Seq. ID No. 5957, Seq. ID No. 5959, Seq. ID No. 5967, Seq. ID No. 5971, Seq. ID No. 5975, Seq. ID No. 5977, Seq. ID No. 5981, Seq. ID No. 5983, Seq. ID No. 5985, Seq. ID No. 5989, Seq. ID No. 5991, Seq. ID No. 5995, Seq. ID No. 5997, Seq. ID No. 6003, Seq. ID No. 6005, Seq. ID No. 6007, Seq. ID No. 6015, Seq. ID No. 6017, Seq. ID No. 6019, Seq. ID No. 6023, Seq. ID No. 6029, Seq. ID No. 6033, Seq. ID No. 6035, Seq. ID No. 6037, Seq. ID No. 6043, Seq. ID No. 6049, Seq. ID No. 6055, Seq. ID No. 6065, Seq. ID No. 6069, Seq. ID No. 6075, Seq. ID No. 6081, Seq. ID No. 6083, Seq. ID No. 6087, Seq. ID No. 6095, Seq. ID No. 6097, Seq. ID No. 6099, Seq. ID No. 6103, Seq. ID No. 6105, Seq. ID No. 6113, Seq. ID No. 6115, Seq. ID No. 6119, Seq. ID No. 6125, Seq. ID No. 6127, Seq. ID No. 6129, Seq. ID No. 6131, Seq. ID No. 6133, Seq. ID No. 6141, Seq. ID No. 6143, Seq. ID No. 6147, Seq. ID No. 6153, Seq. ID No. 6165, Seq. ID No. 6169, Seq. ID No. 6171, Seq. ID No. 6173, Seq. ID No. 6177, Seq. ID No. 6179, Seq. ID No. 6187, Seq. ID No. 6193, Seq. ID No. 6201, Seq. ID No. 6207, Seq. ID No. 6211, Seq. ID No. 6215, Seq. ID No. 6219, Seq. ID No. 6231, Seq. ID No. 6235, Seq. ID No. 6243, Seq. ID No. 6247, Seq. ID No. 6251, Seq. ID No. 6255, Seq. ID No. 6261, Seq. ID No. 6263, Seq. ID No. 6269, Seq. ID No. 6275, Seq. ID No. 6277, Seq. ID No. 6279, Seq. ID No. 6287, Seq. ID No. 6291, Seq. ID No. 6299, Seq. ID No. 6305, Seq. ID No. 6307, Seq. ID No. 6313, Seq. ID No. 6315, Seq. ID No. 6321, Seq. ID No. 6323, Seq. ID No. 6325, Seq. ID No. 6331, Seq. ID No. 6335, Seq. ID No. 6339, Seq. ID No. 6341, Seq. ID No. 6343, Seq. ID No. 6345, Seq. ID No. 6347, Seq. ID No. 6355, Seq. ID No. 6359, Seq. ID No. 6361, Seq. ID No. 6365, Seq. ID No. 6369, Seq. ID No. 6371, Seq. ID No. 6375, Seq. ID No. 6377, Seq. ID No. 6379, Seq. ID No. 6387, Seq. ID No. 6391, Seq. ID No. 6397, Seq. ID No. 6401, Seq. ID No. 6405, Seq. ID No. 6409, Seq. ID No. 6413, Seq. ID No. 6417, Seq. ID No. 6419, Seq. ID No. 6423, Seq. ID No. 6429, Seq. ID No. 6433, Seq. ID No. 6435, Seq. ID No. 6439, Seq. ID No. 6445, Seq. ID No. 6447, Seq. ID No. 6451, Seq. ID No. 6455, Seq. ID No. 6459, Seq. ID No. 6461, Seq. ID No. 6463, Seq. ID No. 6475, Seq. ID No. 6481, Seq. ID No. 6483, Seq. ID No. 6485, Seq. ID No. 6487, Seq. ID No. 6489, Seq. ID No. 6491, Seq. ID No. 6493, Seq. ID No. 6495, Seq. ID No. 6499, Seq. ID No. 6505, Seq. ID No. 6509, Seq. ID No. 6513, Seq. ID No. 6519, Seq. ID No. 6521, Seq. ID No. 6525, Seq. ID No. 6527, Seq. ID No. 6531, Seq. ID No. 6533, Seq. ID No. 6535, Seq. ID No. 6545, Seq. ID No. 6551, Seq. ID No. 6553, Seq. ID No. 6559, Seq. ID No. 6561, Seq. ID No. 6563, Seq. ID No. 6565, Seq. ID No. 6567, Seq. ID No. 6569, Seq. ID No. 6575, Seq. ID No. 6579, Seq. ID No. 6583, Seq. ID No. 6585, Seq. ID No. 6591, Seq. ID No. 6595, Seq. ID No. 6605, Seq. ID No. 6607, Seq. ID No. 6611, Seq. ID No. 6615, Seq. ID No. 6617, Seq. ID No. 6619, Seq. ID No. 6623, Seq. ID No. 6625, Seq. ID No. 6629, Seq. ID No. 6633, Seq. ID No. 6635, Seq. ID No. 6641, and Seq. ID No. 6647.

29. The polynucleotide of claim 7, wherein the polynucleotide that encodes a polypeptide is identified using an ORF finder program that searches for an ATG, GTG or TTG Start codon between a Stop-Stop region, and the polynucleotide is selected from one of Seq. ID No. 1, Seq. ID No. 5, Seq. ID No. 11, Seq. ID No. 13, Seq. ID No. 15, Seq. ID No. 17, Seq. ID No. 19, Seq. ID No. 21, Seq. ID No. 23, Seq. ID No. 27, Seq. ID No. 29, Seq. ID No. 31, Seq. ID No. 35, Seq. ID No. 37, Seq. ID No. 41, Seq. ID No. 43, Seq. ID No. 45, Seq. ID No. 47, Seq. ID No. 49, Seq. ID No. 51, Seq. ID No. 53, Seq. ID No. 55, Seq. ID No. 57, Seq. ID No. 59, Seq. ID No. 65, Seq. ID No. 67, Seq. ID No. 75, Seq. ID No. 77, Seq. ID No. 79, Seq. ID No. 81, Seq. ID No. 83, Seq. ID No. 89, Seq. ID No. 91, Seq. ID No. 93, Seq. ID No. 97, Seq. ID No. 99, Seq. ID No. 103, Seq. ID No. 105, Seq. ID No. 107, Seq. ID No. 109, Seq. ID No. 111, Seq. ID No. 113, Seq. ID No. 115, Seq. ID No. 117, Seq. ID No. 119, Seq. ID No. 121, Seq. ID No. 127, Seq. ID No. 129, Seq. ID No. 131, Seq. ID No. 133, Seq. ID No. 135, Seq. ID No. 137, Seq. ID No. 139, Seq. ID No. 143, Seq. ID No. 145, Seq. ID No. 147, Seq. ID No. 149, Seq. ID No. 153, Seq. ID No. 155, Seq. ID No. 157, Seq. ID No. 159, Seq. ID No. 161, Seq. ID No. 163, Seq. ID No. 165, Seq. ID No. 167, Seq. ID No. 169, Seq. ID No. 171, Seq. ID No. 175, Seq. ID No. 177, Seq. ID No. 179, Seq. ID No. 181, Seq. ID No. 183, Seq. ID No. 187, Seq. ID No. 191, Seq. ID No. 193, Seq. ID No. 195, Seq. ID No. 201, Seq. ID No. 205, Seq. ID No. 207, Seq. ID No. 215, Seq. ID No. 219, Seq. ID No. 221, Seq. ID No. 223, Seq. ID No. 225, Seq. ID No. 227, Seq. ID No. 231, Seq. ID No. 233, Seq. ID No. 239, Seq. ID No. 243, Seq. ID No. 245, Seq. ID No. 247, Seq. ID No. 253, Seq. ID No. 257, Seq. ID No. 259, Seq. ID No. 261, Seq. ID No. 263, Seq. ID No. 265, Seq. ID No. 267, Seq. ID No. 269, Seq. ID No. 273, Seq. ID No. 277, Seq. ID No. 279, Seq. ID No. 281, Seq. ID No. 283, Seq. ID No. 291, Seq. ID No. 293, Seq. ID No. 297, Seq. ID No. 301, Seq. ID No. 305, Seq. ID No. 307, Seq. ID No. 309, Seq. ID No. 313, Seq. ID No. 317, Seq. ID No. 321, Seq. ID No. 323, Seq. ID No. 325, Seq. ID No. 331, Seq. ID No. 333, Seq. ID No. 335, Seq. ID No. 337, Seq. ID No. 339, Seq. ID No. 343, Seq. ID No. 345, Seq. ID No. 347, Seq. ID No. 349, Seq. ID No. 353, Seq. ID No. 355, Seq. ID No. 357, Seq. ID No. 359, Seq. ID No. 361, Seq. ID No. 363, Seq. ID No. 365, Seq. ID No. 367, Seq. ID No. 369, Seq. ID No. 371, Seq. ID No. 375, Seq. ID No. 377, Seq. ID No. 379, Seq. ID No. 383, Seq. ID No. 385, Seq. ID No. 387, Seq. ID No. 389, Seq. ID No. 391, Seq. ID No. 393, Seq. ID No. 397, Seq. ID No. 401, Seq. ID No. 403, Seq. ID No. 405, Seq. ID No. 407, Seq. ID No. 409, Seq. ID No. 415, Seq. ID No. 423, Seq. ID No. 425, Seq. ID No. 427, Seq. ID No. 429, Seq. ID No. 431, Seq. ID No. 433, Seq. ID No. 435, Seq. ID No. 437, Seq. ID No. 439, Seq. ID No. 441, Seq. ID No. 443, Seq. ID No. 445, Seq. ID No. 447, Seq. ID No. 451, Seq. ID No. 453, Seq. ID No. 455, Seq. ID No. 457, Seq. ID No. 461, Seq. ID No. 463, Seq. ID No. 467, Seq. ID No. 469, Seq. ID No. 471, Seq. ID No. 473, Seq. ID No. 475, Seq. ID No. 477, Seq. ID No. 479, Seq. ID No. 481, Seq. ID No. 483, Seq. ID No. 485, Seq. ID No. 489, Seq. ID No. 491, Seq. ID No. 493, Seq. ID No. 495, Seq. ID No. 501, Seq. ID No. 505, Seq. ID No. 507, Seq. ID No. 511, Seq. ID No. 515, Seq. ID No. 517, Seq. ID No. 523, Seq. ID No. 529, Seq. ID No. 531, Seq. ID No. 533, Seq. ID No. 537, Seq. ID No. 539, Seq. ID No. 543, Seq. ID No. 547, Seq. ID No. 549, Seq. ID No. 551, Seq. ID No. 553, Seq. ID No. 555, Seq. ID No. 557, Seq. ID No. 559, Seq. ID No. 561, Seq. ID No. 565, Seq. ID No. 569, Seq. ID No. 571, Seq. ID No. 573, Seq. ID No. 575, Seq. ID No. 579, Seq. ID No. 581, Seq. ID No. 585, Seq. ID No. 587, Seq. ID No. 589, Seq. ID No. 591, Seq. ID No. 593, Seq. ID No. 595, Seq. ID No. 597, Seq. ID No. 599, Seq. ID No. 601, Seq. ID No. 603, Seq. ID No. 605, Seq. ID No. 609, Seq. ID No. 613, Seq. ID No. 615, Seq. ID No. 617, Seq. ID No. 619, Seq. ID No. 621, Seq. ID No. 623, Seq. ID No. 625, Seq. ID No. 627, Seq. ID No. 635, Seq. ID No. 637, Seq. ID No. 643, Seq. ID No. 645, Seq. ID No. 647, Seq. ID No. 649, Seq. ID No. 655, Seq. ID No. 663, Seq. ID No. 665, Seq. ID No. 667, Seq. ID No. 669, Seq. ID No. 675, Seq. ID No. 681, Seq. ID No. 683, Seq. ID No. 687, Seq. ID No. 689, Seq. ID No. 691, Seq. ID No. 695, Seq. ID No. 697, Seq. ID No. 699, Seq. ID No. 701, Seq. ID No. 703, Seq. ID No. 705, Seq. ID No. 707, Seq. ID No. 709, Seq. ID No. 713, Seq. ID No. 725, Seq. ID No. 727, Seq. ID No. 729, Seq. ID No. 731, Seq. ID No. 733, Seq. ID No. 735, Seq. ID No. 739, Seq. ID No. 741, Seq. ID No. 745, Seq. ID No. 747, Seq. ID No. 749, Seq. ID No. 751, Seq. ID No. 753, Seq. ID No. 759, Seq. ID No. 763, Seq. ID No. 765, Seq. ID No. 773, Seq. ID No. 775, Seq. ID No. 781, Seq. ID No. 783, Seq. ID No. 787, Seq. ID No. 793, Seq. ID No. 795, Seq. ID No. 799, Seq. ID No. 801, Seq. ID No. 805, Seq. ID No. 807, Seq. ID No. 809, Seq. ID No. 811, Seq. ID No. 813, Seq. ID No. 815, Seq. ID No. 817, Seq. ID No. 819, Seq. ID No. 821, Seq. ID No. 823, Seq. ID No. 827, Seq. ID No. 829, Seq. ID No. 831, Seq. ID No. 835, Seq. ID No. 837, Seq. ID No. 839, Seq. ID No. 843, Seq. ID No. 845, Seq. ID No. 847, Seq. ID No. 855, Seq. ID No. 859, Seq. ID No. 861, Seq. ID No. 865, Seq. ID No. 867, Seq:ID No. 871, Seq. ID No. 873, Seq. ID No. 875, Seq. ID No. 877, Seq. ID No. 879, Seq. ID No. 881, Seq. ID No. 885, Seq. ID No. 895, Seq. ID No. 897, Seq. ID No. 899, Seq. ID No. 901, Seq. ID No. 903, Seq. ID No. 905, Seq. ID No. 907, Seq. ID No. 909, Seq. ID No. 913, Seq. ID No. 915, Seq. ID No. 921, Seq. ID No. 923, Seq. ID No. 925, Seq. ID No. 931, Seq. ID No. 933, Seq. ID No. 935, Seq. ID No. 937, Seq. ID No. 939, Seq. ID No. 941, Seq. ID No. 943, Seq. ID No. 947, Seq. ID No. 949, Seq. ID No. 951, Seq. ID No. 953, Seq. ID No. 955, Seq. ID No. 957, Seq. ID No. 961, Seq. ID No. 963, Seq. ID No. 965, Seq. ID No. 967, Seq. ID No. 969, Seq. ID No. 971, Seq. ID No. 973, Seq. ID No. 975, Seq. ID No. 981, Seq. ID No. 983, Seq. ID No. 985, Seq. ID No. 987, Seq. ID No. 989, Seq. ID No. 991, Seq. ID No. 993, Seq. ID No. 995, Seq. ID No. 997, Seq. ID No. 1001, Seq. ID No. 1003, Seq. ID No. 1005, Seq. ID No. 1007, Seq. ID No. 1009, Seq. ID No. 1017, Seq. ID No. 1019, Seq. ID No. 1023, Seq. ID No. 1025, Seq. ID No. 1027, Seq. ID No. 1039, Seq. ID No. 1041, Seq. ID No. 1043, Seq. ID No. 1045, Seq. ID No. 1047, Seq. ID No. 1049, Seq. ID No. 1051, Seq. ID No. 1057, Seq. ID No. 1059, Seq. ID No. 1061, Seq. ID No. 1065, Seq. ID No. 1069, Seq. ID No. 1075, Seq. ID No. 1077, Seq. ID No. 1079, Seq. ID No. 1081, Seq. ID No. 1083, Seq. ID No. 1085, Seq. ID No. 1087, Seq. ID No. 1091, Seq. ID No. 1093, Seq. ID No. 1095, Seq. ID No. 1097, Seq. ID No. 1099, Seq. ID No. 1101, Seq. ID No. 1103, Seq. ID No. 1107, Seq. ID No. 1109, Seq. ID No. 1111, Seq. ID No. 1113, Seq. ID No. 1115, Seq. ID No. 1119, Seq. ID No. 1121, Seq. ID No. 1123, Seq. ID No. 1125, Seq. ID No. 1127, Seq. ID No. 1129, Seq. ID No. 1131, Seq. ID No. 1137, Seq. ID No. 1139, Seq. ID No. 1141, Seq. ID No. 1143, Seq. ID No. 1145, Seq. ID No. 1147, Seq. ID No. 1149, Seq. ID No. 1151, Seq. ID No. 1153, Seq. ID No. 1155, Seq. ID No. 1157, Seq. ID No. 1159, Seq. ID No. 1161, Seq. ID No. 1163, Seq. ID No. 1171, Seq. ID No. 1175, Seq. ID No. 1177, Seq. ID No. 1179, Seq. ID No. 1181, Seq. ID No. 1187, Seq. ID No. 1191, Seq. ID No. 1193, Seq. ID No. 1195, Seq. ID No. 1197, Seq. ID No. 1201, Seq. ID No. 1203, Seq. ID No. 1205, Seq. ID No. 1207, Seq. ID No. 1211, Seq. ID No. 1213, Seq. ID No. 1215, Seq. ID No. 1217, Seq. ID No. 1221, Seq. ID No. 1223, Seq. ID No. 1225, Seq. ID No. 1231, Seq. ID No. 1233, Seq. ID No. 1237, Seq. ID No. 1239, Seq. ID No. 1241, Seq. ID No. 1245, Seq. ID No. 1247, Seq. ID No. 1249, Seq. ID No. 1251, Seq. ID No. 1253, Seq. ID No. 1255, Seq. ID No. 1257, Seq. ID No. 1259, Seq. ID No. 1261, Seq. ID No. 1263, Seq. ID No. 1271, Seq. ID No. 1273, Seq. ID No. 1275, Seq. ID No. 1277, Seq. ID No. 1279, Seq. ID No. 1285, Seq. ID No. 1289, Seq. ID No. 1291, Seq. ID No. 1293, Seq. ID No. 1295, Seq. ID No. 1297, Seq. ID No. 1299, Seq. ID No. 1301, Seq. ID No. 1303, Seq. ID No. 1305, Seq. ID No. 1307, Seq. ID No. 1309, Seq. ID No. 1311, Seq. ID No. 1313, Seq. ID No. 1317, Seq. ID No. 1319, Seq. ID No. 1321, Seq. ID No. 1331, Seq. ID No. 1333, Seq. ID No. 1335, Seq. ID No. 1337, Seq. ID No. 1339, Seq. ID No. 1343, Seq. ID No. 1345, Seq. ID No. 1347, Seq. ID No. 1349, Seq. ID No. 1351, Seq. ID No. 1359, Seq. ID No. 1361, Seq. ID No. 1363, Seq. ID No. 1365, Seq. ID No. 1369, Seq. ID No. 1371, Seq. ID No. 1375, Seq. ID No. 1377, Seq. ID No. 1381, Seq. ID No. 1383, Seq. ID No. 1385, Seq. ID No. 1387, Seq. ID No. 1389, Seq. ID No. 1391, Seq. ID No. 1393, Seq. ID No. 1401, Seq. ID No. 1403, Seq. ID No. 1407, Seq. ID No. 1409, Seq. ID No. 1411, Seq. ID No. 1413, Seq. ID No. 1415, Seq. ID No. 1419, Seq. ID No. 1421, Seq. ID No. 1423, Seq. ID No. 1425, Seq. ID No. 1427, Seq. ID No. 1429, Seq. ID No. 1433, Seq. ID No. 1437, Seq. ID No. 1441, Seq. ID No. 1443, Seq. ID No. 1447, Seq. ID No. 1449, Seq. ID No. 1451, Seq. ID No. 1455, Seq. ID No. 1459, Seq. ID No. 1463, Seq. ID No. 1465, Seq. ID No. 1467, Seq. ID No. 1469, Seq. ID No. 1471, Seq. ID No. 1473, Seq. ID No. 1475, Seq. ID No. 1477, Seq. ID No. 1479, Seq. ID No. 1483, Seq. ID No. 1485, Seq. ID No. 1487, Seq. ID No. 1491, Seq. ID No. 1493, Seq. ID No. 1495, Seq. ID No. 1497, Seq. ID No. 1499, Seq. ID No. 1501, Seq. ID No. 1505, Seq. ID No. 1507, Seq. ID No. 1511, Seq. ID No. 1517, Seq. ID No. 1519, Seq. ID No. 1523, Seq. ID No. 1525, Seq. ID No. 1527, Seq. ID No. 1531, Seq. ID No. 1539, Seq. ID No. 1543, Seq. ID No. 1549, Seq. ID No. 1551, Seq. ID No. 1553, Seq. ID No. 1555, Seq. ID No. 1557, Seq. ID No. 1561, Seq. ID No. 1563, Seq. ID No. 1565, Seq. ID No. 1567, Seq. ID No. 1571, Seq. ID No. 1573, Seq. ID No. 1575, Seq. ID No. 1577, Seq. ID No. 1579, Seq. ID No. 1581, Seq. ID No. 1589, Seq. ID No. 1593, Seq. ID No. 1595, Seq. ID No. 1597, Seq. ID No. 1599, Seq. ID No. 1601, Seq. ID No. 1607, Seq. ID No. 1611, Seq. ID No. 1613, Seq. ID No. 1615, Seq. ID No. 1617, Seq. ID No. 1619, Seq. ID No. 1623, Seq. ID No. 1625, Seq. ID No. 1627, Seq. ID No. 1633, Seq. ID No. 1637, Seq. ID No. 1639, Seq. ID No. 1641, Seq. ID No. 1643, Seq. ID No. 1647, Seq. ID No. 1651, Seq. ID No. 1653, Seq. ID No. 1655, Seq. ID No. 1657, Seq. ID No. 1661, Seq. ID No. 1663, Seq. ID No. 1665, Seq. ID No. 1669, Seq. ID No. 1675, Seq. ID No. 1679, Seq. ID No. 1681, Seq. ID No. 1685, Seq. ID No. 1687, Seq. ID No. 1689, Seq. ID No. 1691, Seq. ID No. 1693, Seq. ID No. 1695, Seq. ID No. 1697, Seq. ID No. 1699, Seq. ID No. 1703, Seq. ID No. 1705, Seq. ID No. 1707, Seq. ID No. 1711, Seq. ID No. 1713, Seq. ID No. 1715, Seq. ID No. 1717, Seq. ID No. 1719, Seq. ID No. 1721, Seq. ID No. 1723, Seq. ID No. 1727, Seq. ID No. 1729, Seq. ID No. 1735, Seq. ID No. 1737, Seq. ID No. 1739, Seq. ID No. 1741, Seq. ID No. 1747, Seq. ID No. 1749, Seq. ID No. 1751, Seq. ID No. 1755, Seq. ID No. 1757, Seq. ID No. 1759, Seq. ID No. 1763, Seq. ID No. 1769, Seq. ID No. 1771, Seq. ID No. 1775, Seq. ID No. 1777, Seq. ID No. 1779, Seq. ID No. 1781, Seq. ID No. 1783, Seq. ID No. 1785, Seq. ID No. 1789, Seq. ID No. 1791, Seq. ID No. 1793, Seq. ID No. 1795, Seq. ID No. 1797, Seq. ID No. 1805, Seq. ID No. 1809, Seq. ID No. 1811, Seq. ID No. 1813, Seq. ID No. 1815, Seq. ID No. 1817, Seq. ID No. 1819, Seq. ID No. 1821, Seq. ID No. 1823, Seq. ID No. 1825, Seq. ID No. 1829, Seq. ID No. 1833, Seq. ID No. 1841, Seq. ID No. 1843, Seq. ID No. 1845, Seq. ID No. 1847, Seq. ID No. 1849, Seq. ID No. 1853, Seq. ID No. 1855, Seq. ID No. 1857, Seq. ID No. 1859, Seq. ID No. 1861, Seq. ID No. 1863, Seq. ID No. 1865, Seq. ID No. 1867, Seq. ID No. 1871, Seq. ID No. 1873, Seq. ID No. 1875, Seq. ID No. 1877, Seq. ID No. 1881, Seq. ID No. 1883, Seq. ID No. 1885, Seq. ID No. 1887, Seq. ID No. 1889, Seq. ID No. 1895, Seq. ID No. 1903, Seq. ID No. 1905, Seq. ID No. 1907, Seq. ID No. 1911, Seq. ID No. 1913, Seq. ID No. 1915, Seq. ID No. 1919, Seq. ID No. 1923, Seq. ID No. 1925, Seq. ID No. 1927, Seq. ID No. 1929, Seq. ID No. 1931, Seq. ID No. 1935, Seq. ID No. 1941, Seq. ID No. 1943, Seq. ID No. 1945, Seq. ID No. 1947, Seq. ID No. 1949, Seq. ID No. 1951, Seq. ID No. 1953, Seq. ID No. 1959, Seq. ID No. 1961, Seq. ID No. 1963, Seq. ID No. 1965, Seq. ID No. 1967, Seq. ID No. 1969, Seq. ID No. 1971, Seq. ID No. 1979, Seq. ID No. 1983, Seq. ID No. 1985, Seq. ID No. 1987, Seq. ID No. 1989, Seq. ID No. 1991, Seq. ID No. 1995, Seq. ID No. 1997, Seq. ID No. 1999, Seq. ID No. 2001, Seq. ID No. 2003, Seq. ID No. 2005, Seq. ID No. 2007, Seq. ID No. 2015, Seq. ID No. 2019, Seq. ID No. 2021, Seq. ID No. 2025, Seq. ID No. 2027, Seq. ID No. 2029, Seq. ID No. 2031, Seq. ID No. 2035, Seq. ID No. 2037, Seq. ID No. 2041, Seq. ID No. 2043, Seq. ID No. 2045, Seq. ID No. 2047, Seq. ID No. 2053, Seq. ID No. 2055, Seq. ID No. 2059, Seq. ID No. 2063, Seq. ID No. 2065, Seq. ID No. 2067, Seq. ID No. 2069, Seq. ID No. 2071, Seq. ID No. 2073, Seq. ID No. 2075, Seq. ID No. 2077, Seq. ID No. 2079, Seq. ID No. 2081, Seq. ID No. 2083, Seq. ID No. 2085, Seq. ID No. 2089, Seq. ID No. 2091, Seq. ID No. 2093, Seq. ID No. 2097, Seq. ID No. 2099, Seq. ID No. 2101, Seq. ID No. 2103, Seq. ID No. 2107, Seq. ID No. 2109, Seq. ID No. 2111, Seq. ID No. 2115, Seq. ID No. 2117, Seq. ID No. 2119, Seq. ID No. 2125, Seq. ID No. 2127, Seq. ID No. 2129, Seq. ID No. 2131, Seq. ID No. 2133, Seq. ID No. 2135, Seq. ID No. 2137, Seq. ID No. 2141, Seq. ID No. 2143, Seq. ID No. 2145, Seq. ID No. 2149, Seq. ID No. 2151, Seq. ID No. 2153, Seq. ID No. 2155, Seq. ID No. 2157, Seq. ID No. 2159, Seq. ID No. 2161, Seq. ID No. 2165, Seq. ID No. 2167, Seq. ID No. 2169, Seq. ID No. 2179, Seq. ID No. 2181, Seq. ID No. 2183, Seq. ID No. 2189, Seq. ID No. 2191, Seq. ID No. 2195, Seq. ID No. 2197, Seq. ID No. 2199, Seq. ID No. 2201, Seq. ID No. 2203, Seq. ID No. 2205, Seq. ID No. 2207, Seq. ID No. 2209, Seq. ID No. 2211, Seq. ID No. 2213, Seq. ID No. 2215, Seq. ID No. 2217, Seq. ID No. 2221, Seq. ID No. 2223, Seq. ID No. 2227, Seq. ID No. 2229, Seq. ID No. 2235, Seq. ID No. 2241, Seq. ID No. 2245, Seq. ID No. 2247, Seq. ID No. 2251, Seq. ID No. 2253, Seq. ID No. 2257, Seq. ID No. 2259, Seq. ID No. 2261, Seq. ID No. 2263, Seq. ID No. 2265, Seq. ID No. 2267, Seq. ID No. 2269, Seq. ID No. 2275, Seq. ID No. 2277, Seq. ID No. 2279, Seq. ID No. 2285, Seq. ID No. 2287, Seq. ID No. 2297, Seq. ID No. 2299, Seq. ID No. 2301, Seq. ID No. 2309, Seq. ID No. 2311, Seq. ID No. 2315, Seq. ID No. 2327, Seq. ID No. 2329, Seq. ID No. 2333, Seq. ID No. 2343, Seq. ID No. 2345, Seq. ID No. 2349, Seq. ID No. 2351, Seq. ID No. 2357, Seq. ID No. 2363, Seq. ID No. 2365, Seq. ID No. 2367, Seq. ID No. 2369, Seq. ID No. 2371, Seq. ID No. 2373, Seq. ID No. 2375, Seq. ID No. 2379, Seq. ID No. 2381, Seq. ID No. 2385, Seq. ID No. 2387, Seq. ID No. 2391, Seq. ID No. 2393, Seq. ID No. 2395, Seq. ID No. 2397, Seq. ID No. 2399, Seq. ID No. 2401, Seq. ID No. 2405, Seq. ID No. 2409, Seq. ID No. 2413, Seq. ID No. 2415, Seq. ID No. 2417, Seq. ID No. 2421, Seq. ID No. 2423, Seq. ID No. 2425, Seq. ID No. 2427, Seq. ID No. 2429, Seq. ID No. 2435, Seq. ID No. 2437, Seq. ID No. 2445, Seq. ID No. 2447, Seq. ID No. 2449, Seq. ID No. 2451, Seq. ID No. 2453, Seq. ID No. 2455, Seq. ID No. 2457, Seq. ID No. 2459, Seq. ID No. 2461, Seq. ID No. 2463, Seq. ID No. 2465, Seq. ID No. 2469, Seq. ID No. 2471, Seq. ID No. 2475, Seq. ID No. 2481, Seq. ID No. 2483, Seq. ID No. 2485, Seq. ID No. 2491, Seq. ID No. 2493, Seq. ID No. 2495, Seq. ID No. 2499, Seq. ID No. 2501, Seq. ID No. 2503, Seq. ID No. 2505, Seq. ID No. 2507, Seq. ID No. 2511, Seq. ID No. 2513, Seq. ID No. 2517, Seq. ID No. 2519, Seq. ID No. 2521, Seq. ID No. 2525, Seq. ID No. 2529, Seq. ID No. 2533, Seq. ID No. 2535, Seq. ID No. 2537, Seq. ID No. 2539, Seq. ID No. 2547, Seq. ID No. 2549, Seq. ID No. 2551, Seq. ID No. 2553, Seq. ID No. 2559, Seq. ID No. 2561, Seq. ID No. 2563, Seq. ID No. 2565, Seq. ID No. 2573, Seq. ID No. 2575, Seq. ID No. 2577, Seq. ID No. 2579, Seq. ID No. 2581, Seq. ID No. 2587, Seq. ID No. 2589, Seq. ID No. 2591, Seq. ID No. 2593, Seq. ID No. 2595, Seq. ID No. 2599, Seq. ID No. 2603, Seq. ID No. 2605, Seq. ID No. 2607, Seq. ID No. 2609, Seq. ID No. 2611, Seq. ID No. 2613, Seq. ID No. 2617, Seq. ID No. 2619, Seq. ID No. 2623, Seq. ID No. 2625, Seq. ID No. 2627, Seq. ID No. 2629, Seq. ID No. 2631, Seq. ID No. 2633, Seq. ID No. 2635, Seq. ID No. 2637, Seq. ID No. 2639, Seq. ID No. 2641, Seq. ID No. 2643, Seq. ID No. 2645, Seq. ID No. 2647, Seq. ID No. 2649, Seq. ID No. 2651, Seq. ID No. 2653, Seq. ID No. 2655, Seq. ID No. 2659, Seq. ID No. 2663, Seq. ID No. 2667, Seq. ID No. 2669, Seq. ID No. 2671, Seq. ID No. 2675, Seq. ID No. 2677, Seq. ID No. 2683, Seq. ID No. 2685, Seq. ID No. 2687, Seq. ID No. 2689, Seq. ID No. 2691, Seq. ID No. 2693, Seq. ID No. 2695, Seq. ID No. 2697, Seq. ID No. 2699, Seq. ID No. 2703, Seq. ID No. 2709, Seq. ID No. 2711, Seq. ID No. 2715, Seq. ID No. 2717, Seq. ID No. 2719, Seq. ID No. 2723, Seq. ID No. 2725, Seq. ID No. 2727, Seq. ID No. 2729, Seq. ID No. 2731, Seq. ID No. 2735, Seq. ID No. 2737, Seq. ID No. 2739, Seq. ID No. 2749, Seq. ID No. 2751, Seq. ID No. 2753, Seq. ID No. 2755, Seq. ID No. 2759, Seq. ID No. 2765, Seq. ID No. 2767, Seq. ID No. 2769, Seq. ID No. 2771, Seq. ID No. 2773, Seq. ID No. 2775, Seq. ID No. 2779, Seq. ID No. 2781, Seq. ID No. 2783, Seq. ID No. 2787, Seq. ID No. 2789, Seq. ID No. 2791, Seq. ID No. 2793, Seq. ID No. 2795, Seq. ID No. 2797, Seq. ID No. 2799, Seq. ID No. 2801, Seq. ID No. 2803, Seq. ID No. 2807, Seq. ID No. 2809, Seq. ID No. 2813, Seq. ID No. 2815, Seq. ID No. 2821, Seq. ID No. 2823, Seq. ID No. 2827, Seq. ID No. 2829, Seq. ID No. 2831, Seq. ID No. 2833, Seq. ID No. 2839, Seq. ID No. 2841, Seq. ID No. 2843, Seq. ID No. 2845, Seq. ID No. 2847, Seq. ID No. 2855, Seq. ID No. 2861, Seq. ID No. 2863, Seq. ID No. 2865, Seq. ID No. 2867, Seq. ID No. 2869, Seq. ID No. 2871, Seq. ID No. 2877, Seq. ID No. 2881, Seq. ID No. 2883, Seq. ID No. 2885, Seq. ID No. 2887, Seq. ID No. 2891, Seq. ID No. 2893, Seq. ID No. 2895, Seq. ID No. 2899, Seq. ID No. 2901, Seq. ID No. 2907, Seq. ID No. 2915, Seq. ID No. 2917, Seq. ID No. 2919, Seq. ID No. 2921, Seq. ID No. 2923, Seq. ID No. 2927, Seq. ID No. 2931, Seq. ID No. 2933, Seq. ID No. 2935, Seq. ID No. 2943, Seq. ID No. 2945, Seq. ID No. 2951, Seq. ID No. 2953, Seq. ID No. 2957, Seq. ID No. 2959, Seq. ID No. 2961, Seq. ID No. 2963, Seq. ID No. 2965, Seq. ID No. 2967, Seq. ID No. 2971, Seq. ID No. 2973, Seq. ID No. 2975, Seq. ID No. 2977, Seq. ID No. 2979, Seq. ID No. 2983, Seq. ID No. 2985, Seq. ID No. 2987, Seq. ID No. 2991, Seq. ID No. 2993, Seq. ID No. 2999, Seq. ID No. 3001, Seq. ID No. 3005, Seq. ID No. 3009, Seq. ID No. 3011, Seq. ID No. 3015, Seq. ID No. 3017, Seq. ID No. 3019, Seq. ID No. 3021, Seq. ID No. 3027, Seq. ID No. 3033, Seq. ID No. 3035, Seq. ID No. 3037, Seq. ID No. 3039, Seq. ID No. 3043, Seq. ID No. 3045, Seq. ID No. 3047, Seq. ID No. 3049, Seq. ID No. 3051, Seq. ID No. 3053, Seq. ID No. 3055, Seq. ID No. 3057, Seq. ID No. 3059, Seq. ID No. 3067, Seq. ID No. 3069, Seq. ID No. 3071, Seq. ID No. 3079, Seq. ID No. 3083, Seq. ID No. 3085, Seq. ID No. 3087, Seq. ID No. 3091, Seq. ID No. 3093, Seq. ID No. 3099, Seq. ID No. 3101, Seq. ID No. 3103, Seq. ID No. 3109, Seq. ID No. 3111, Seq. ID No. 3115, Seq. ID No. 3117, Seq. ID No. 3119, Seq. ID No. 3121, Seq. ID No. 3123, Seq. ID No. 3125, Seq. ID No. 3129, Seq. ID No. 3131, Seq. ID No. 3133, Seq. ID No. 3135, Seq. ID No. 3137, Seq. ID No. 3139, Seq. ID No. 3141, Seq. ID No. 3143, Seq. ID No. 3147, Seq. ID No. 3149, Seq. ID No. 3151, Seq. ID No. 3153, Seq. ID No. 3155, Seq. ID No. 3157, Seq. ID No. 3159, Seq. ID No. 3161, Seq. ID No. 3165, Seq. ID No. 3167, Seq. ID No. 3169, Seq. ID No. 3171, Seq. ID No. 3173, Seq. ID No. 3179, Seq. ID No. 3181, Seq. ID No. 3183, Seq. ID No. 3185, Seq. ID No. 3187, Seq. ID No. 3189, Seq. ID No. 3191, Seq. ID No. 3193, Seq. ID No. 3195, Seq. ID No. 3197, Seq. ID No. 3203, Seq. ID No. 3205, Seq. ID No. 3207, Seq. ID No. 3209, Seq. ID No. 3211, Seq. ID No. 3217, Seq. ID No. 3223, Seq. ID No. 3225, Seq. ID No. 3227, Seq. ID No. 3229, Seq. ID No. 3235, Seq. ID No. 3249, Seq. ID No. 3251, Seq. ID No. 3253, Seq. ID No. 3255, Seq. ID No. 3257, Seq. ID No. 3259, Seq. ID No. 3263, Seq. ID No. 3265, Seq. ID No. 3269, Seq. ID No. 3271, Seq. ID No. 3273, Seq. ID No. 3277, Seq. ID No. 3281, Seq. ID No. 3283, Seq. ID No. 3285, Seq. ID No. 3289, Seq. ID No. 3291, Seq. ID No. 3293, Seq. ID No. 3299, Seq. ID No. 3301, Seq. ID No. 3303, Seq. ID No. 3305, Seq. ID No. 3307, Seq. ID No. 3309, Seq. ID No. 3311, Seq. ID No. 3313, Seq. ID No. 3315, Seq. ID No. 3319, Seq. ID No. 3321, Seq. ID No. 3323, Seq. ID No. 3325, Seq. ID No. 3331, Seq. ID No. 3335, Seq. ID No. 3337, Seq. ID No. 3339, Seq. ID No. 3341, Seq. ID No. 3345, Seq. ID No. 3347, Seq. ID No. 3349, Seq. ID No. 3353, Seq. ID No. 3355, Seq. ID No. 3359, Seq. ID No. 3361, Seq. ID No. 3363, Seq. ID No. 3365, Seq. ID No. 3367, Seq. ID No. 3369, Seq. ID No. 3371, Seq. ID No. 3373, Seq. ID No. 3375, Seq. ID No. 3377, Seq. ID No. 3379, Seq. ID No. 3381, Seq. ID No. 3383, Seq. ID No. 3397, Seq. ID No. 3399, Seq. ID No. 3401, Seq. ID No. 3407, Seq. ID No. 3409, Seq. ID No. 3413, Seq. ID No. 3415, Seq. ID No. 3419, Seq. ID No. 3425, Seq. ID No. 3427, Seq. ID No. 3431, Seq. ID No. 3433, Seq. ID No. 3435, Seq. ID No. 3439, Seq. ID No. 3441, Seq. ID No. 3443, Seq. ID No. 3445, Seq. ID No. 3447, Seq. ID No. 3455, Seq. ID No. 3457, Seq. ID No. 3459, Seq. ID No. 3461, Seq. ID No. 3465, Seq. ID No. 3467, Seq. ID No. 3471, Seq. ID No. 3473, Seq. ID No. 3475, Seq. ID No. 3479, Seq. ID No. 3483, Seq. ID No. 3485, Seq. ID No. 3487, Seq. ID No. 3489, Seq. ID No. 3495, Seq. ID No. 3497, Seq. ID No. 3499, Seq. ID No. 3501, Seq. ID No. 3505, Seq. ID No. 3507, Seq. ID No. 3509, Seq. ID No. 3511, Seq. ID No. 3513, Seq. ID No. 3517, Seq. ID No. 3519, Seq. ID No. 3521, Seq. ID No. 3525, Seq. ID No. 3529, Seq. ID No. 3531, Seq. ID No. 3533, Seq. ID No. 3535, Seq. ID No. 3537, Seq. ID No. 3539, Seq. ID No. 3541, Seq. ID No. 3543, Seq. ID No. 3545, Seq. ID No. 3547, Seq. ID No. 3549, Seq. ID No. 3551, Seq. ID No. 3553, Seq. ID No. 3555, Seq. ID No. 3557, Seq. ID No. 3559, Seq. ID No. 3561, Seq. ID No. 3563, Seq. ID No. 3565, Seq. ID No. 3567, Seq. ID No. 3569, Seq. ID No. 3571, Seq. ID No. 3573, Seq. ID No. 3575, Seq. ID No. 3579, Seq. ID No. 3581, Seq. ID No. 3583, Seq. ID No. 3587, Seq. ID No. 3589, Seq. ID No. 3595, Seq. ID No. 3597, Seq. ID No. 3599, Seq. ID No. 3603, Seq. ID No. 3605, Seq. ID No. 3607, Seq. ID No. 3609, Seq. ID No. 3611, Seq. ID No. 3613, Seq. ID No. 3615, Seq. ID No. 3617, Seq. ID No. 3619, Seq. ID No. 3621, Seq. ID No. 3623, Seq. ID No. 3627, Seq. ID No. 3629, Seq. ID No. 3635, Seq. ID No. 3637, Seq. ID No. 3639, Seq. ID No. 3641, Seq. ID No. 3645, Seq. ID No. 3647, Seq. ID No. 3649, Seq. ID No. 3651, Seq. ID No. 3653, Seq. ID No. 3657, Seq. ID No. 3659, Seq. ID No. 3663, Seq. ID No. 3665, Seq. ID No. 3667, Seq. ID No. 3671, Seq. ID No. 3673, Seq. ID No. 3675, Seq. ID No. 3677, Seq. ID No. 3679, Seq. ID No. 3681, Seq. ID No. 3683, Seq. ID No. 3685, Seq. ID No. 3687, Seq. ID No. 3689, Seq. ID No. 3691, Seq. ID No. 3693, Seq. ID No. 3695, Seq. ID No. 3699, Seq. ID No. 3701, Seq. ID No. 3703, Seq. ID No. 3705, Seq. ID No. 3707, Seq. ID No. 3709, Seq. ID No. 3713, Seq. ID No. 3715, Seq. ID No. 3717, Seq. ID No. 3727, Seq. ID No. 3729, Seq. ID No. 3733, Seq. ID No. 3737, Seq. ID No. 3739, Seq. ID No. 3741, Seq. ID No. 3745, Seq. ID No. 3747, Seq. ID No. 3759, Seq. ID No. 3767, Seq. ID No. 3769, Seq. ID No. 3771, Seq. ID No. 3779, Seq. ID No. 3783, Seq. ID No. 3785, Seq. ID No. 3789, Seq. ID No. 3791, Seq. ID No. 3795, Seq. ID No. 3797, Seq. ID No. 3803, Seq. ID No. 3805, Seq. ID No. 3807, Seq. ID No. 3809, Seq. ID No. 3811, Seq. ID No. 3813, Seq. ID No. 3815, Seq. ID No. 3817, Seq. ID No. 3819, Seq. ID No. 3823, Seq. ID No. 3825, Seq. ID No. 3831, Seq. ID No. 3833, Seq. ID No. 3835, Seq. ID No. 3841, Seq. ID No. 3843, Seq. ID No. 3847, Seq. ID No. 3855, Seq. ID No. 3857, Seq. ID No. 3863, Seq. ID No. 3867, Seq. ID No. 3871, Seq. ID No. 3873, Seq. ID No. 3875, Seq. ID No. 3877, Seq. ID No. 3881, Seq. ID No. 3883, Seq. ID No. 3885, Seq. ID No. 3893, Seq. ID No. 3895, Seq. ID No. 3899, Seq. ID No. 3901, Seq. ID No. 3905, Seq. ID No. 3907, Seq. ID No. 3911, Seq. ID No. 3913, Seq. ID No. 3915, Seq. ID No. 3919, Seq. ID No. 3921, Seq. ID No. 3923, Seq. ID No. 3925, Seq. ID No. 3927, Seq. ID No. 3929, Seq. ID No. 3931, Seq. ID No. 3933, Seq. ID No. 3935, Seq. ID No. 3939, Seq. ID No. 3941, Seq. ID No. 3943, Seq. ID No. 3945, Seq. ID No. 3947, Seq. ID No. 3949, Seq. ID No. 3951, Seq. ID No. 3953, Seq. ID No. 3955, Seq. ID No. 3957, Seq. ID No. 3959, Seq. ID No. 3961, Seq. ID No. 3965, Seq. ID No. 3967, Seq. ID No. 3969, Seq. ID No. 3971, Seq. ID No. 3975, Seq. ID No. 3979, Seq. ID No. 3981, Seq. ID No. 3983, Seq. ID No. 3985, Seq. ID No. 3987, Seq. ID No. 3991, Seq. ID No. 3995, Seq. ID No. 3997, Seq. ID No. 3999, Seq. ID No. 4003, Seq. ID No. 4007, Seq. ID No. 4009, Seq. ID No. 4015, Seq. ID No. 4017, Seq. ID No. 4021, Seq. ID No. 4023, Seq. ID No. 4029, Seq. ID No. 4035, Seq. ID No. 4037, Seq. ID No. 4039, Seq. ID No. 4049, Seq. ID No. 4051, Seq. ID No. 4058, Seq. ID No. 4055, Seq. ID No. 4057, Seq. ID No. 4061, Seq. ID No. 4075, Seq. ID No. 4079, Seq. ID No. 4085, Seq. ID No. 4087, Seq. ID No. 4089, Seq. ID No. 4091, Seq. ID No. 4093, Seq. ID No. 4095, Seq. ID No. 4099, Seq. ID No. 4105, Seq. ID No. 4107, Seq. ID No. 4109, Seq. ID No. 4111, Seq. ID No. 4113, Seq. ID No. 4117, Seq. ID No. 4119, Seq. ID No. 4121, Seq. ID No. 4123, Seq. ID No. 4125, Seq. ID No. 4127, Seq. ID No. 4129, Seq. ID No. 4131, Seq. ID No. 4133, Seq. ID No. 4135, Seq. ID No. 4137, Seq. ID No. 4139, Seq. ID No. 4141, Seq. ID No. 4143, Seq. ID No. 4145, Seq. ID No. 4149, Seq. ID No. 4153, Seq. ID No. 4155, Seq. ID No. 4157, Seq. ID No. 4161, Seq. ID No. 4163, Seq. ID No. 4167, Seq. ID No. 4171, Seq. ID No. 4173, Seq. ID No. 4175, Seq. ID No. 4177, Seq. ID No. 4179, Seq. ID No. 4181, Seq. ID No. 4185, Seq. ID No. 4187, Seq. ID No. 4189, Seq. ID No. 4191, Seq. ID No. 4193, Seq. ID No. 4195, Seq. ID No. 4197, Seq. ID No. 4199, Seq. ID No. 4203, Seq. ID No. 4205, Seq. ID No. 4207, Seq. ID No. 4209, Seq. ID No. 4211, Seq. ID No. 4219, Seq. ID No. 4229, Seq. ID No. 4233, Seq. ID No. 4235, Seq. ID No. 4237, Seq. ID No. 4239, Seq. ID No. 4243, Seq. ID No. 4249, Seq. ID No. 4251, Seq. ID No. 4253, Seq. ID No. 4255, Seq. ID No. 4259, Seq. ID No. 4263, Seq. ID No. 4265, Seq. ID No. 4267, Seq. ID No. 4269, Seq. ID No. 4271, Seq. ID No. 4273, Seq. ID No. 4275, Seq. ID No. 4281, Seq. ID No. 4283, Seq. ID No. 4285, Seq. ID No. 4287, Seq. ID No. 4289, Seq. ID No. 4291, Seq. ID No. 4293, Seq. ID No. 4295, Seq. ID No. 4297, Seq. ID No. 4299, Seq. ID No. 4301, Seq. ID No. 4303, Seq. ID No. 4307, Seq. ID No. 4309, Seq. ID No. 4313, Seq. ID No. 4315, Seq. ID No. 4317, Seq. ID No. 4319, Seq. ID No. 4321, Seq. ID No. 4323, Seq. ID No. 4325, Seq. ID No. 4329, Seq. ID No. 4331, Seq. ID No. 4333, Seq. ID No. 4335, Seq. ID No. 4337, Seq. ID No. 4339, Seq. ID No. 4343, Seq. ID No. 4345, Seq. ID No. 4347, Seq. ID No. 4351, Seq. ID No. 4353, Seq. ID No. 4355, Seq. ID No. 4359, Seq. ID No. 4361, Seq. ID No. 4363, Seq. ID No. 4365, Seq. ID No. 4367, Seq. ID No. 4369, Seq. ID No. 4375, Seq. ID No. 4377, Seq. ID No. 4379, Seq. ID No. 4383, Seq. ID No. 4389, Seq. ID No. 4391, Seq. ID No. 4393, Seq. ID No. 4395, Seq. ID No. 4397, Seq. ID No. 4399, Seq. ID No. 4401, Seq. ID No. 4403, Seq. ID No. 4405, Seq. ID No. 4407, Seq. ID No. 4409, Seq. ID No. 4411, Seq. ID No. 4413, Seq. ID No. 4415, Seq. ID No. 4417, Seq. ID No. 4419, Seq. ID No. 4421, Seq. ID No. 4423, Seq. ID No. 4425, Seq. ID No. 4427, Seq. ID No. 4429, Seq. ID No. 4431, Seq. ID No. 4435, Seq. ID No. 4437, Seq. ID No. 4439, Seq. ID No. 4441, Seq. ID No. 4449,.Seq. ID No. 4451, Seq. ID No. 4453, Seq. ID No. 4455, Seq. ID No. 4457, Seq. ID No. 4459, Seq. ID No. 4461, Seq. ID No. 4465, Seq. ID No. 4467, Seq. ID No. 4471, Seq. ID No. 4475, Seq. ID No. 4477, Seq. ID No. 4479, Seq. ID No. 4481, Seq. ID No. 4485, Seq. ID No. 4487, Seq. ID No. 4489, Seq. ID No. 4491, Seq. ID No. 4493, Seq. ID No. 4495, Seq. ID No. 4497, Seq. ID No. 4501, Seq. ID No. 4503, Seq. ID No. 4505, Seq. ID No. 4507, Seq. ID No. 4509, Seq. ID No. 4511, Seq. ID No. 4513, Seq. ID No. 4515, Seq. ID No. 4519, Seq. ID No. 4521, Seq. ID No. 4523, Seq. ID No. 4525, Seq. ID No. 4527, Seq. ID No. 4529, Seq. ID No. 4531, Seq. ID No. 4533, Seq. ID No. 4537, Seq. ID No. 4541, Seq. ID No. 4545, Seq. ID No. 4547, Seq. ID No. 4549, Seq. ID No. 4551, Seq. ID No. 4553, Seq. ID No. 4555, Seq. ID No. 4559, Seq. ID No. 4561, Seq. ID No. 4565, Seq. ID No. 4567, Seq. ID No. 4571, Seq. ID No. 4573, Seq. ID No. 4575, Seq. ID No. 4577, Seq. ID No. 4581, Seq. ID No. 4585, Seq. ID No. 4589, Seq. ID No. 4591, Seq. ID No. 4597, Seq. ID No. 4599, Seq. ID No. 4601, Seq. ID No. 4603, Seq. ID No. 4605, Seq. ID No. 4607, Seq. ID No. 4609, Seq. ID No. 4611, Seq. ID No. 4613, Seq. ID No. 4615, Seq. ID No. 4617, Seq. ID No. 4619, Seq. ID No. 4621, Seq. ID No. 4623, Seq. ID No. 4627, Seq. ID No. 4631, Seq. ID No. 4637, Seq. ID No. 4641, Seq. ID No. 4643, Seq. ID No. 4645, Seq. ID No. 4647, Seq. ID No. 4649, Seq. ID No. 4653, Seq. ID No. 4659, Seq. ID No. 4661, Seq. ID No. 4663, Seq. ID No. 4665, Seq. ID No. 4667, Seq. ID No. 4669, Seq. ID No. 4671, Seq. ID No. 4673, Seq. ID No. 4675, Seq. ID No. 4677, Seq. ID No. 4679, Seq. ID No. 4683, Seq. ID No. 4685, Seq. ID No. 4687, Seq. ID No. 4691, Seq. ID No. 4693, Seq. ID No. 4695, Seq. ID No. 4699, Seq. ID No. 4701, Seq. ID No. 4703, Seq. ID No. 4705, Seq. ID No. 4713, Seq. ID No. 4717, Seq. ID No. 4719, Seq. ID No. 4721, Seq. ID No. 4727, Seq. ID No. 4729, Seq. ID No. 4733, Seq. ID No. 4735, Seq. ID No. 4737, Seq. ID No. 4739, Seq. ID No. 4741, Seq. ID No. 4743, Seq. ID No. 4747, Seq. ID No. 4749, Seq. ID No. 4751, Seq. ID No. 4755, Seq. ID No. 4757, Seq. ID No. 4763, Seq. ID No. 4765, Seq. ID No. 4767, Seq. ID No. 4769, Seq. ID No. 4773, Seq. ID No. 4775, Seq. ID No. 4777, Seq. ID No. 4779, Seq. ID No. 4781, Seq. ID No. 4787, Seq. ID No. 4789, Seq. ID No. 4791, Seq. ID No. 4793, Seq. ID No. 4795, Seq. ID No. 4797, Seq. ID No. 4801, Seq. ID No. 4803, Seq. ID No. 4805, Seq. ID No. 4809, Seq. ID No. 4811, Seq. ID No. 4813, Seq. ID No. 4815, Seq. ID No. 4817, Seq. ID No. 4819, Seq. ID No. 4825, Seq. ID No. 4827, Seq. ID No. 4829, Seq. ID No. 4831, Seq. ID No. 4833, Seq. ID No. 4837, Seq. ID No. 4839, Seq. ID No. 4841, Seq. ID No. 4843, Seq. ID No. 4845, Seq. ID No. 4849, Seq. ID No. 4851, Seq. ID No. 4853, Seq. ID No. 4855, Seq. ID No. 4861, Seq. ID No. 4863, Seq. ID No. 4867, Seq. ID No. 4875, Seq. ID No. 4877, Seq. ID No. 4879, Seq. ID No. 4881, Seq. ID No. 4883, Seq. ID No. 4889, Seq. ID No. 4891, Seq. ID No. 4893, Seq. ID No. 4895, Seq. ID No. 4897, Seq;. ID No. 4899, Seq. ID No. 4901, Seq. ID No. 4903, Seq. ID No. 4905, Seq. ID No. 4907, Seq. ID No. 4909, Seq. ID No. 4911, Seq. ID No. 4913, Seq. ID No. 4915, Seq. ID No. 4917, Seq. ID No. 4919, Seq. ID No. 4921, Seq. ID No. 4923, Seq. ID No. 4927, Seq. ID No. 4931, Seq. ID No. 4935, Seq. ID No. 4937, Seq. ID No. 4939, Seq. ID No. 4941, Seq. ID No. 4943, Seq. ID No. 4945, Seq. ID No. 4947, Seq. ID No. 4949, Seq. ID No. 4951, Seq. ID No. 4953, Seq. ID No. 4957, Seq. ID No. 4959, Seq. ID No. 4961, Seq. ID No. 4963, Seq. ID No. 4973, Seq. ID No. 4975, Seq. ID No. 4977, Seq. ID No. 4979, Seq. ID No. 4981, Seq. ID No. 4983, Seq. ID No. 4985, Seq. ID No. 4987, Seq. ID No. 4989, Seq. ID No. 4991, Seq. ID No. 4993, Seq. ID No. 4995, Seq. ID No. 4997, Seq. ID No. 4999, Seq. ID No. 5003, Seq. ID No. 5005, Seq. ID No. 5007, Seq. ID No. 5009, Seq. ID No. 5011, Seq. ID No. 5013, Seq. ID No. 5017, Seq. ID No. 5019, Seq. ID No. 5021, Seq. ID No. 5023, Seq. ID No. 5025, Seq. ID No. 5029, Seq. ID No. 5031, Seq. ID No. 5033, Seq. ID No. 5035, Seq. ID No. 5041, Seq. ID No. 5043, Seq. ID No. 5045, Seq. ID No. 5047, Seq. ID No. 5057, Seq. ID No. 5059, Seq. ID No. 5063, Seq. ID No. 5065, Seq. ID No. 5067, Seq. ID No. 5069, Seq. ID No. 5075, Seq. ID No. 5077, Seq. ID No. 5079, Seq. ID No. 5081, Seq. ID No. 5083, Seq. ID No. 5085, Seq. ID No. 5087, Seq. ID No. 5091, Seq. ID No. 5099, Seq. ID No. 5101, Seq. ID No. 5103, Seq. ID No. 5109, Seq. ID No. 5111, Seq. ID No. 5113, Seq. ID No. 5115, Seq. ID No. 5117, Seq. ID No. 5121, Seq. ID No. 5125, Seq. ID No. 5127, Seq. ID No. 5133, Seq. ID No. 5139, Seq. ID No. 5141, Seq. ID No. 5145, Seq. ID No. 5147, Seq. ID No. 5149, Seq. ID No. 5151, Seq. ID No. 5155, Seq. ID No. 5157, Seq. ID No. 5159, Seq. ID No. 5161, Seq. ID No. 5165, Seq. ID No. 5167, Seq. ID No. 5169, Seq. ID No. 5177, Seq. ID No. 5181, Seq. ID No. 5183, Seq. ID No. 5185, Seq. ID No. 5187, Seq. ID No. 5189, Seq. ID No. 5191, Seq. ID No. 5193, Seq. ID No. 5195, Seq. ID No. 5197, Seq. ID No. 5199, Seq. ID No. 5201, Seq. ID No. 5203, Seq. ID No. 5205, Seq. ID No. 5209, Seq. ID No. 5211, Seq. ID No. 5215, Seq. ID No. 5219, Seq. ID No. 5221, Seq. ID No. 5229, Seq. ID No. 5231, Seq. ID No. 5239, Seq. ID No. 5241, Seq. ID No. 5243, Seq. ID No. 5245, Seq. ID No. 5251, Seq. ID No. 5253, Seq. ID No. 5255, Seq. ID No. 5261, Seq. ID No. 5263, Seq. ID No. 5265, Seq. ID No. 5267, Seq. ID No. 5277, Seq. ID No. 5279, Seq. ID No. 5281, Seq. ID No. 5285, Seq. ID No. 5287, Seq. ID No. 5291, Seq. ID No. 5295, Seq. ID No. 5297, Seq. ID No. 5299, Seq. ID No. 5301, Seq. ID No. 5303, Seq. ID No. 5305, Seq. ID No. 5307, Seq. ID No. 5309, Seq. ID No. 5311, Seq. ID No. 5313, Seq. ID No. 5317, Seq. ID No. 5319, Seq. ID No. 5321, Seq. ID No. 5323, Seq. ID No. 5327, Seq. ID No. 5329, Seq. ID No. 5331, Seq. ID No. 5333, Seq. ID No. 5335, Seq. ID No. 5337, Seq. ID No. 5343, Seq. ID No. 5345, Seq. ID No. 5349, Seq. ID No. 5351, Seq. ID No. 5357, Seq. ID No. 5359, Seq. ID No. 5365, Seq. ID No. 5367, Seq. ID No. 5373, Seq. ID No. 5377, Seq. ID No. 5379, Seq. ID No. 5381, Seq. ID No. 5383, Seq. ID No. 5387, Seq. ID No. 5389, Seq. ID No. 5391, Seq. ID No. 5393, Seq. ID No. 5397, Seq. ID No. 5399, Seq. ID No. 5401, Seq. ID No. 5403, Seq. ID No. 5405, Seq. ID No. 5407, Seq. ID No. 5409, Seq. ID No. 5411, Seq. ID No. 5415, Seq. ID No. 5417, Seq. ID No. 5419, Seq. ID No. 5421, Seq. ID No. 5423, Seq. ID No. 5425, Seq. ID No. 5427, Seq. ID No. 5431, Seq. ID No. 5433, Seq. ID No. 5435, Seq. ID No. 5437, Seq. ID No. 5439, Seq. ID No. 5441, Seq. ID No. 5443, Seq. ID No. 5447, Seq. ID No. 5449, Seq. ID No. 5451, Seq. ID No. 5453, Seq. ID No. 5455, Seq. ID No. 5457, Seq. ID No. 5467, Seq. ID No. 5471, Seq. ID No. 5473, Seq. ID No. 5477, Seq. ID No. 5479, Seq: ID No. 5481, Seq. ID No. 5491, Seq. ID No. 5493, Seq. ID No. 5495, Seq. ID No. 5499, Seq. ID No. 5503, Seq. ID No. 5509, Seq. ID No. 5513, Seq. ID No. 5515, Seq. ID No. 5517, Seq. ID No. 5519, Seq. ID No. 5521, Seq. ID No. 5527, Seq. ID No. 5529, Seq. ID No. 5531, Seq. ID No. 5533, Seq. ID No. 5537, Seq. ID No. 5539, Seq. ID No. 5541, Seq. ID No. 5543, Seq. ID No. 5545, Seq. ID No. 5547, Seq. ID No. 5549, Seq. ID No. 5551, Seq. ID No. 5553, Seq. ID No. 5557, Seq. ID No. 5559, Seq. ID No. 5563, Seq. ID No. 5567, Seq. ID No. 5569, Seq. ID No. 5575, Seq. ID No. 5577, Seq. ID No. 5581, Seq. ID No. 5583, Seq. ID No. 5585, Seq. ID No. 5589, Seq. ID No. 5591, Seq. ID No. 5593, Seq. ID No. 5595, Seq. ID No. 5599, Seq. ID No. 5601, Seq. ID No. 5607, Seq. ID No. 5613, Seq. ID No. 5615, Seq. ID No. 5617, Seq. ID No. 5619, Seq. ID No. 5621, Seq. ID No. 5625, Seq. ID No. 5627, Seq. ID No. 5629, Seq. ID No. 5631, Seq. ID No. 5637, Seq. ID No. 5639, Seq. ID No. 5641, Seq. ID No. 5645, Seq. ID No. 5647, Seq. ID No. 5649, Seq. ID No. 5651, Seq. ID No. 5655 Seq. ID No. 5657, Seq. ID No. 5661, Seq. ID No. 5665, Seq. ID No. 5667, Seq. ID No. 5669, Seq. ID No. 5677, Seq. ID No. 5679, Seq. ID No. 5683, Seq. ID No. 5685, Seq. ID No. 5687, Seq. ID No. 5689, Seq. ID No. 5691, Seq. ID No. 5693, Seq. ID No. 5697, Seq. ID No. 5699, Seq. ID No. 5701, Seq. ID No. 5703, Seq. ID No. 5705, Seq. ID No. 5707, Seq. ID No. 5711, Seq. ID No. 5713, Seq. ID No. 5715, Seq. ID No. 5717, Seq. ID No. 5719, Seq. ID No. 5721, Seq. ID No. 5723, Seq. ID No. 5725, Seq. ID No. 5727, Seq. ID No. 5735, Seq. ID No. 5739, Seq. ID No. 5741, Seq. ID No. 5743, Seq. ID No. 5745, Seq. ID No. 5747, Seq. ID No. 5749, Seq. ID No. 5751, Seq. ID No. 5753, Seq. ID No. 5755, Seq. ID No. 5757, Seq. ID No. 5759, Seq. ID No. 5761, Seq. ID No. 5769, Seq. ID No. 5771, Seq. ID No. 5777, Seq. ID No. 5779, Seq. ID No. 5783, Seq. ID No. 5787, Seq. ID No. 5791, Seq. ID No. 5795, Seq. ID No. 5799, Seq. ID No. 5801, Seq. ID No. 5807, Seq. ID No. 5809, Seq. ID No. 5813, Seq. ID No. 5815, Seq. ID No. 5819, Seq. ID No. 5821, Seq. ID No. 5823, Seq. ID No. 5825, Seq. ID No. 5827, Seq. ID No. 5829, Seq. ID No. 5831, Seq. ID No. 5833, Seq. ID No. 5835, Seq. ID No. 5837, Seq. ID No. 5839, Seq. ID No. 5841, Seq. ID No. 5845, Seq. ID No. 5847, Seq. ID No. 5849, Seq. ID No. 5851, Seq. ID No. 5855, Seq. ID No. 5857, Seq. ID No. 5859, Seq. ID No. 5861, Seq. ID No. 5863, Seq. ID No. 5865, Seq. ID No. 5869, Seq. ID No. 5871, Seq. ID No. 5873, Seq. ID No. 5875, Seq. ID No. 5879, Seq. ID No. 5881, Seq. ID No. 5883, Seq. ID No. 5889, Seq. ID No. 5895, Seq. ID No. 5897, Seq. ID No. 5901, Seq. ID No. 5903, Seq. ID No. 5911, Seq. ID No. 5913, Seq. ID No. 5915, Seq. ID No. 5917, Seq. ID No. 5919, Seq. ID No. 5927, Seq. ID No. 5929, Seq. ID No. 5933, Seq. ID No. 5935, Seq. ID No. 5937, Seq. ID No. 5941, Seq. ID No. 5945, Seq. ID No. 5947, Seq. ID No. 5951, Seq. ID No. 5959, Seq. ID No. 5961, Seq. ID No. 5963, Seq. ID No. 5965, Seq. ID No. 5967, Seq. ID No. 5969, Seq. ID No. 5971, Seq. ID No. 5973, Seq. ID No. 5977, Seq. ID No. 5979, Seq. ID No. 5981, Seq. ID No. 5985, Seq. ID No. 5987, Seq. ID No. 5989, Seq. ID No. 5991, Seq. ID No. 5993, Seq. ID No. 5995, Seq. ID No. 5999, Seq. ID No. 6001, Seq. ID No. 6003, Seq. ID No. 6009, Seq. ID No. 6011, Seq. ID No. 6013, Seq. ID No. 6015, Seq. ID No. 6017, Seq. ID No. 6025, Seq. ID No. 6027, Seq. ID No. 6029, Seq. ID No. 6031, Seq. ID No. 6033, Seq. ID No. 6035, Seq. ID No. 6039, Seq. ID No. 6041, Seq. ID No. 6043, Seq. ID No. 6045, Seq. ID No. 6049, Seq. ID No. 6051, Seq. ID No. 6053, Seq. ID No. 6055, Seq. ID No. 6057, Seq. ID No. 6061, Seq. ID No. 6063, Seq. ID No. 6065, Seq. ID No. 6069, Seq. ID No. 6071, Seq. ID No. 6073, Seq. ID No. 6077, Seq. ID No. 6079, Seq. ID No. 6085, Seq. ID No. 6087, Seq. ID No. 6089, Seq. ID No. 6093, Seq. ID No. 6095, Seq. ID No. 6101, Seq. ID No. 6103, Seq. ID No. 6109, Seq. ID No. 6111, Seq. ID No. 6117, Seq. ID No. 6121, Seq. ID No. 6123, Seq. ID No. 6129, Seq. ID No. 6131, Seq. ID No. 6133, Seq. ID No. 6135, Seq. ID No. 6137, Seq. ID No. 6139, Seq. ID No. 6141, Seq. ID No. 6145, Seq. ID No. 6151, Seq. ID No. 6155, Seq. ID No. 6157, Seq. ID No. 6159, Seq. ID No. 6165, Seq. ID No. 6167, Seq. ID No. 6169, Seq. ID No. 6171, Seq. ID No. 6175, Seq. ID No. 6177, Seq. ID No. 6181, Seq. ID No. 6187, Seq. ID No. 6193, Seq. ID No. 6195, Seq. ID No. 6197, Seq. ID No. 6199, Seq. ID No. 6201, Seq. ID No. 6209, Seq. ID No. 6213, Seq. ID No. 6215, Seq. ID No. 6217, Seq. ID No. 6219, Seq. ID No. 6231, Seq. ID No. 6233, Seq. ID No. 6235, Seq. ID No. 6239, Seq. ID No. 6241, Seq. ID No. 6243, Seq. ID No. 6249, Seq. ID No. 6257, Seq. ID No. 6259, Seq. ID No. 6261, Seq. ID No. 6263, Seq. ID No. 6267, Seq. ID No. 6269, Seq. ID No. 6271, Seq. ID No. 6275, Seq. ID No. 6277, Seq. ID No. 6279, Seq. ID No. 6281, Seq. ID No. 6283, Seq. ID No. 6285, Seq. ID No. 6287, Seq. ID No. 6293, Seq. ID No. 6295, Seq. ID No. 6297, Seq. ID No. 6299, Seq. ID No. 6305, Seq. ID No. 6309, Seq. ID No. 6311, Seq. ID No. 6315, Seq. ID No. 6317, Seq. ID No. 6319, Seq. ID No. 6321, Seq. ID No. 6323, Seq. ID No. 6325, Seq. ID No. 6327, Seq. ID No. 6329, Seq. ID No. 6331, Seq. ID No. 6335, Seq. ID No. 6337, Seq. ID No. 6341, Seq. ID No. 6345, Seq. ID No. 6349, Seq. ID No. 6351, Seq. ID No. 6353, Seq. ID No. 6359, Seq. ID No. 6361, Seq. ID No. 6367, Seq. ID No; 6369, Seq. ID No. 6371, Seq. ID No. 6373, Seq. ID No. 6375, Seq. ID No. 6377, Seq. ID No. 6379, Seq. ID No. 6381, Seq. ID No. 6385, Seq. ID No. 6389, Seq. ID No. 6393, Seq. ID No. 6395, Seq. ID No. 6399, Seq. ID No. 6409, Seq. ID No. 6411, Seq. ID No. 6413, Seq. ID No. 6417, Seq. ID No. 6421, Seq. ID No. 6423, Seq. ID No. 6425, Seq. ID No. 6427, Seq. ID No. 6429, Seq. ID No. 6431, Seq. ID No. 6441, Seq. ID No. 6443, Seq. ID No. 6445, Seq. ID No. 6451, Seq. ID No. 6453, Seq. ID No. 6459, Seq. ID No. 6461, Seq. ID No. 6463, Seq. ID No. 6465, Seq. ID No. 6467, Seq. ID No. 6469, Seq. ID No. 6471, Seq. ID No. 6473, Seq. ID No. 6479, Seq. ID No. 6483, Seq. ID No. 6493, Seq. ID No. 6495, Seq. ID No. 6497, Seq. ID No. 6501, Seq. ID No. 6503, Seq. ID No. 6505, Seq. ID No. 6507, Seq. ID No. 6511, Seq. ID No. 6517, Seq. ID No. 6523, Seq. ID No. 6525, Seq. ID No. 6535, Seq. ID No. 6537, Seq. ID No. 6539, Seq. ID No. 6541, Seq. ID No. 6543, Seq. ID No. 6545, Seq. ID No. 6547, Seq. ID No. 6549, Seq. ID No. 6551, Seq. ID No. 6553, Seq. ID No. 6555, Seq. ID No. 6557, Seq. ID No. 6559, Seq. ID No. 6563, Seq. ID No. 6573, Seq. ID No. 6575, Seq. ID No. 6577, Seq. ID No. 6579, Seq. ID No. 6583, Seq. ID No. 6585, Seq. ID No. 6587, Seq. ID No. 6591, Seq. ID No. 6597, Seq. ID No. 6601, Seq. ID No. 6603, Seq. ID No. 6605, Seq. ID No. 6609, Seq. ID No. 6611, Seq. ID No. 6613, Seq. ID No. 6615, Seq. ID No. 6617, Seq. ID No. 6619, Seq. ID No. 6621, Seq. ID No. 6623, Seq. ID No. 6627, Seq. ID No. 6629, Seq. ID No. 6631, Seq. ID No. 6633, Seq. ID No. 6637, Seq. ID No. 6639, Seq. ID No. 6643, Seq. ID No. 6645, and Seq. ID No. 6649.

30. The polynucleotide of claim 7 wherein the polynucleotide encodes a polypeptide that is identified by an ORF finder program, which searches for a transmembrane domain between two Stop codons and a Start codon immediately upstream of this transmembrane region, and the polynucleotide is selected from one of Seq. ID No. 7, Seq. ID No. 9, Seq. ID No. 33, Seq. ID No. 47, Seq. ID No. 67, Seq. ID No. 85, Seq. ID No. 87, Seq. ID No. 123, Seq. ID No. 133, Seq. ID No. 141, Seq. ID No. 159, Seq. ID No. 163, Seq. ID No. 191, Seq. ID No. 209, Seq. ID No. 211, Seq. ID No. 215, Seq. ID No. 217, Seq. ID No. 225, Seq. ID No. 235, Seq. ID No. 237, Seq. ID No. 249, Seq. ID No. 251, Seq. ID No. 255, Seq. ID No. 271, Seq. ID No. 277., Seq. ID No. 285, Seq. ID No. 287, Seq. ID No. 289, Seq. ID No. 327, Seq. ID No. 329, Seq. ID No. 373, Seq. ID No. 375, Seq. ID No. 419, Seq. ID No. 487, Seq. ID No. 497, Seq. ID No. 499, Seq. ID No. 503, Seq. ID No. 505, Seq. ID No. 509, Seq. ID No. 511, Seq. ID No. 515, Seq. ID No. 525, Seq. ID No. 527, Seq. ID No. 545, Seq. ID No. 567, Seq. ID No. 569, Seq. ID No. 575, Seq. ID No. 577, Seq. ID No. 579, Seq. ID No. 593, Seq. ID No. 617, Seq. ID No. 637, Seq. ID No. 639, Seq. ID No. 641, Seq. ID No. 645, Seq. ID No. 653, Seq. ID No. 655, Seq. ID No. 659, Seq. ID No. 669, Seq. ID No. 675, Seq. ID No. 677, Seq. ID No. 679, Seq. ID No. 715, Seq. ID No. 717, Seq. ID No. 719, Seq. ID No. 721, Seq. ID No. 723, Seq. ID No. 747, Seq. ID No. 751, Seq. ID No. 755, Seq. ID No. 757, Seq. ID No. 759, Seq. ID No. 761, Seq. ID No. 763, Seq. ID No. 769, Seq. ID No. 777, Seq. ID No. 779, Seq. ID No. 807, Seq. ID No. 809, Seq. ID No. 819, Seq. ID No. 825, Seq. ID No. 863, Seq. ID No. 893, Seq. ID No. 901, Seq. ID No. 935, Seq. ID No. 943, Seq. ID No. 955, Seq. ID No. 967, Seq. ID No. 979, Seq. ID No. 1005, Seq. ID No. 1009, Seq. ID No. 1011, Seq. ID No. 1013, Seq. ID No. 1015, Seq. ID No. 1019, Seq. ID No. 1029, Seq. ID No. 1031, Seq. ID No. 1033, Seq. ID No. 1037, Seq. ID No. 1039, Seq. ID No. 1045, Seq. ID No. 1055, Seq. ID No. 1075, Seq. ID No. 1105, Seq. ID No. 1131, Seq. ID No. 1135, Seq. ID No. 1137, Seq. ID No. 1165, Seq. ID No. 1167, Seq. ID No. 1169, Seq. ID No. 1183, Seq. ID No. 1193, Seq. ID No. 1197, Seq. ID No. 1213, Seq. ID No. 1223, Seq. ID No. 1227, Seq. ID No. 1229, Seq. ID No. 1237, Seq. ID No. 1243, Seq. ID No. 1265, Seq. ID No. 1267, Seq. ID No. 1269, Seq. ID No. 1271, Seq. ID No. 1283, Seq. ID No. 1287, Seq. ID No. 1291, Seq. ID No. 1297, Seq. ID No. 1305, Seq. ID No. 1315, Seq. ID No. 1325, Seq. ID No. 1327, Seq. ID No. 1329, Seq. ID No. 1331, Seq. ID No. 1341, Seq. ID No. 1353, Seq. ID No. 1367, Seq. ID No. 1401, Seq. ID No. 1429, Seq. ID No. 1435, Seq. ID No. 1445, Seq. ID No. 1481, Seq. ID No. 1513, Seq. ID No. 1515, Seq. ID No. 1517, Seq. ID No. 1529, Seq. ID No. 1533, Seq. ID No. 1535, Seq. ID No. 1545, Seq. ID No. 1547, Seq. ID No. 1551, Seq. ID No. 1559, Seq. ID No. 1563, Seq. ID No. 1571, Seq. ID No. 1579, Seq. ID No. 1583, Seq. ID No. 1585, Seq. ID No. 1621, Seq. ID No. 1633, Seq. ID No. 1643, Seq. ID No. 1645, Seq. ID No. 1647, Seq. ID No. 1649, Seq. ID No. 1653, Seq. ID No. 1671, Seq. ID No. 1673, Seq. ID No. 1677, Seq. ID No. 1681, Seq. ID No. 1707, Seq. ID No. 1731, Seq. ID No. 1733, Seq. ID No. 1737, Seq. ID No. 1753, Seq. ID No. 1757, Seq. ID No. 1765, Seq. ID No. 1767, Seq. ID No. 1787, Seq. ID No. 1791, Seq. ID No. 1797, Seq. ID No. 1805, Seq. ID No. 1819, Seq. ID No. 1821, Seq. ID No. 1823, Seq. ID No. 1839, Seq. ID No. 1869, Seq. ID No. 1875, Seq. ID No. 1891, Seq. ID No. 1893, Seq. ID No. 1895, Seq. ID No. 1923, Seq. ID No. 1927, Seq. ID No. 1969, Seq. ID No. 1981, Seq. ID No. 1991, Seq. ID No. 1993, Seq. ID No. 2013, Seq. ID No. 2017, Seq. ID No. 2027, Seq. ID No. 2033, Seq. ID No. 2041, Seq. ID No. 2049, Seq. ID No. 2053, Seq. ID No. 2087, Seq. ID No. 2089, Seq. ID No. 2105, Seq. ID No. 2115, Seq. ID No. 2121, Seq. ID No. 2127, Seq. ID No. 2151, Seq. ID No. 2159, Seq. ID No. 2171, Seq. ID No. 2173, Seq. ID No. 2177, Seq. ID No. 2187, Seq. ID No. 2237, Seq. ID No. 2239, Seq. ID No. 2245, Seq. ID No. 2249, Seq. ID No. 2271, Seq. ID No. 2283, Seq. ID No. 2289, Seq. ID No. 2291, Seq. ID No. 2293, Seq. ID No. 2295, Seq. ID No. 2303, Seq. ID No. 2305, Seq. ID No. 2307, Seq. ID No. 2317, Seq. ID No. 2319, Seq. ID No. 2321, Seq. ID No. 2323, Seq. ID No. 2325, Seq. ID No. 2327, Seq. ID No. 2333, Seq. ID No. 2343, Seq. ID No. 2347, Seq. ID No. 2349, Seq. ID No. 2351, Seq. ID No. 2353, Seq. ID No. 2355, Seq. ID No. 2357, Seq. ID No. 2365, Seq. ID No. 2389, Seq. ID No. 2391, Seq. ID No. 2407, Seq. ID No. 2419, Seq. ID No. 2421, Seq. ID No. 2427, Seq. ID No. 2429, Seq. ID No. 2433, Seq. ID No. 2449, Seq. ID No. 2459, Seq. ID No. 2489, Seq. ID No. 2497, Seq. ID No. 2503, Seq. ID No. 2507, Seq. ID No. 2515, Seq. ID No. 2523, Seq. ID No. 2525, Seq. ID No. 2531, Seq. ID No. 2535, Seq. ID No. 2537, Seq. ID No. 2541, Seq. ID No. 2543, Seq. ID No. 2545, Seq. ID No. 2555, Seq. ID No. 2557, Seq. ID No. 2569, Seq. ID No. 2571, Seq. ID No. 2583, Seq. ID No. 2591, Seq. ID No. 2597, Seq. ID No. 2599, Seq. ID No. 2601, Seq. ID No. 2607, Seq. ID No. 2611, Seq. ID No. 2621, Seq. ID No. 2629, Seq. ID No. 2651, Seq. ID No. 2679, Seq. ID No. 2693, Seq. ID No. 2701, Seq. ID No. 2703, Seq. ID No. 2705, Seq. ID No. 2707, Seq. ID No. 2731, Seq. ID No. 2733, Seq. ID No. 2783, Seq. ID No. 2785, Seq. ID No. 2813, Seq. ID No. 2815, Seq. ID No. 2823, Seq. ID No. 2835, Seq. ID No. 2837, Seq. ID No. 2841, Seq. ID No. 2853, Seq. ID No. 2873, Seq. ID No. 2875, Seq. ID No. 2881, Seq. ID No. 2905, Seq. ID No. 2935, Seq. ID No. 2937, Seq. ID No. 2939, Seq. ID No. 2941, Seq. ID No. 2947, Seq. ID No. 2949, Seq. ID No. 2969, Seq. ID No. 2973, Seq. ID No. 2977, Seq. ID No. 2981, Seq. ID No. 2987, Seq. ID No. 2991, Seq. ID No. 2995, Seq. ID No. 2997, Seq. ID No. 3005, Seq. ID No. 3007, Seq. ID No. 3013, Seq. ID No. 3023, Seq. ID No. 3025, Seq. ID No. 3027, Seq. ID No. 3029, Seq. ID No. 3031, Seq. ID No. 3043, Seq. ID No. 3055, Seq. ID No. 3063, Seq. ID No. 3071, Seq. ID No. 3073, Seq. ID No. 3075, Seq. ID No. 3077, Seq. ID No. 3123, Seq. ID No. 3145, Seq. ID No. 3155, Seq. ID No. 3157, Seq. ID No. 3163, Seq. ID No. 3183, Seq. ID No. 3193, Seq. ID No. 3201, Seq. ID No. 3213, Seq. ID No. 3215, Seq. ID No. 3217, Seq. ID No. 3219, Seq. ID No. 3241, Seq. ID No. 3243, Seq. ID No. 3245, Seq. ID No. 3249, Seq. ID No. 3257, Seq. ID No. 3261, Seq. ID No. 3269, Seq. ID No. 3275, Seq. ID No. 3297, Seq. ID No. 3317, Seq. ID No. 3323, Seq. ID No. 3337, Seq. ID No. 3339, Seq. ID No. 3349, Seq. ID No. 3351, Seq. ID No. 3353, Seq. ID No. 3357, Seq. ID No. 3359, Seq. ID No. 3377, Seq. ID No. 3389, Seq. ID No. 3391, Seq. ID No. 3395, Seq. ID No. 3405, Seq. ID No. 3421, Seq. ID No. 3425, Seq. ID No. 3449, Seq. ID No. 3451, Seq. ID No. 3453, Seq. ID No. 3455, Seq. ID No. 3457, Seq. ID No. 3463, Seq. ID No. 3481, Seq. ID No. 3491, Seq. ID No. 3493, Seq. ID No. 3503, Seq. ID No. 3505, Seq. ID No. 3527, Seq. ID No. 3593, Seq. ID No. 3595, Seq. ID No. 3621, Seq. ID No. 3625, Seq. ID No. 3635, Seq. ID No. 3679, Seq. ID No. 3723, Seq. ID No. 3727, Seq. ID No. 3745, Seq. ID No. 3753, Seq. ID No. 3755, Seq. ID No. 3757, Seq. ID No. 3761, Seq. ID No. 3763, Seq. ID No. 3765, Seq. ID No. 3783, Seq. ID No. 3793, Seq. ID No. 3799, Seq. ID No. 3813, Seq. ID No. 3837, Seq. ID No. 3839, Seq. ID No. 3849, Seq. ID No. 3851, Seq. ID No. 3853, Seq. ID No. 3859, Seq. ID No. 3861, Seq. ID No. 3863, Seq. ID No. 3865, Seq. ID No. 3879, Seq. ID No. 3889, Seq. ID No. 3891, Seq. ID No. 3903, Seq. ID No. 3925, Seq. ID No. 3963, Seq. ID No. 3973, Seq. ID No. 3975, Seq. ID No. 4005, Seq. ID No. 4025, Seq. ID No. 4027, Seq. ID No. 4039, Seq. ID No. 4041, Seq. ID No. 4043, Seq. ID No. 4045, Seq. ID No. 4047, Seq. ID No. 4061, Seq. ID No. 4071, Seq. ID No. 4073, Seq. ID No. 4079, Seq. ID No. 4081, Seq. ID No. 4083, Seq. ID No. 4101, Seq. ID No. 4103, Seq. ID No. 4111, Seq. ID No. 4133, Seq. ID No. 4137, Seq. ID No. 4139, Seq. ID No. 4145, Seq. ID No. 4151, Seq. ID No. 4221, Seq. ID No. 4223, Seq. ID No. 4225, Seq. ID No. 4227, Seq. ID No. 4231, Seq. ID No. 4241, Seq. ID No. 4247, Seq. ID No. 4253, Seq. ID No. 4257, Seq. ID No. 4261, Seq. ID No. 4307, Seq. ID No. 4311, Seq. ID No. 4313, Seq. ID No. 4343, Seq. ID No. 4379, Seq. ID No. 4385, Seq. ID No. 4389, Seq. ID No. 4439, Seq. ID No. 4469, Seq. ID No. 4473, Seq. ID No. 4481, Seq. ID No. 4483, Seq. ID No. 4485, Seq. ID No. 4497, Seq. ID No. 4515, Seq. ID No. 4533, Seq. ID No. 4587, Seq. ID No. 4639, Seq. ID No. 4643, Seq. ID No. 4681, Seq. ID No. 4705, Seq. ID No. 4707, Seq. ID No. 4709, Seq. ID No. 4711, Seq. ID No. 4715, Seq. ID No. 4717, Seq. ID No. 4737, Seq. ID No. 4759, Seq. ID No. 4813, Seq. ID No. 4827, Seq. ID No. 4869, Seq. ID No. 4871, Seq. ID No. 4873, Seq. ID No. 4875, Seq. ID No. 4919, Seq. ID No. 4931, Seq. ID No. 4949, Seq. ID No. 4951, Seq. ID No. 4987, Seq. ID No. 5031, Seq. ID No. 5061, Seq. ID No. 5085, Seq. ID No. 5103, Seq. ID No. 5123, Seq. ID No. 5129, Seq. ID No. 5131, Seq. ID No. 5135, Seq. ID No. 5137, Seq. ID No. 5141, Seq. ID No. 5165, Seq. ID No. 5175, Seq. ID No. 5199, Seq. ID No. 5205, Seq. ID No. 5207, Seq. ID No. 5213, Seq. ID No. 5219, Seq. ID No. 5223, Seq. ID No. 5267, Seq. ID No. 5281, Seq. ID No. 5385, Seq. ID No. 5387, Seq. ID No. 5413, Seq. ID No. 5415, Seq. ID No. 5433, Seq. ID No. 5453, Seq. ID No. 5459, Seq. ID No. 5461, Seq. ID No. 5463, Seq. ID No. 5465, Seq. ID No. 5491, Seq. ID No. 5497, Seq. ID No. 5509, Seq. ID No. 5513, Seq. ID No. 5525, Seq. ID No. 5531, Seq. ID No. 5569, Seq. ID No. 5587, Seq. ID No. 5595, Seq. ID No. 5597, Seq. ID No. 5609, Seq. ID No. 5611, Seq. ID No. 5621, Seq. ID No. 5623, Seq. ID No. 5633, Seq. ID No. 5637, Seq. ID No. 5643, Seq. ID No. 5649, Seq. ID No. 5659, Seq. ID No. 5683, Seq. ID No. 5685, Seq. ID No. 5699, Seq. ID No. 5705, Seq. ID No. 5709, Seq. ID No. 5729, Seq. ID No. 5733, Seq. ID No. 5763, Seq. ID No. 5765, Seq. ID No. 5767, Seq. ID No. 5771, Seq. ID No. 5783, Seq. ID No. 5791, Seq. ID No. 5797, Seq. ID No. 5885, Seq. ID No. 5889, Seq. ID No. 5893, Seq. ID No. 5895, Seq. ID No. 5899, Seq. ID No. 5901, Seq. ID No. 5905, Seq. ID No. 5909, Seq. ID No. 5921, Seq. ID No. 5923, Seq. ID No. 5927, Seq. ID No. 5939, Seq. ID No. 6027, Seq. ID No. 6043, Seq. ID No. 6047, Seq. ID No. 6059, Seq. ID No. 6067, Seq. ID No. 6089, Seq. ID No. 6091, Seq. ID No. 6107, Seq. ID No. 6111, Seq. ID No. 6117, Seq. ID No. 6161, Seq. ID No. 6163, Seq. ID No. 6165, Seq. ID No. 6169, Seq. ID No. 6183, Seq. ID No. 6185, Seq. ID No. 6189, Seq. ID No. 6191, Seq. ID No. 6197, Seq. ID No. 6203, Seq. ID No. 6205, Seq. ID No. 6209, Seq. ID No. 6221, Seq. ID No. 6223, Seq. ID No. 6225, Seq. ID No. 6227, Seq. ID No. 6229, Seq. ID No. 6235, Seq. ID No. 6237, Seq. ID No. 6243, Seq. ID No. 6245, Seq. ID No. 6249, Seq. ID No. 6253, Seq. ID No. 6263, Seq. ID No. 6265, Seq. ID No. 6273, Seq. ID No. 6275, Seq. ID No. 6285, Seq. ID No. 6289, Seq. ID No. 6301, Seq. ID No. 6303, Seq. ID No. 6363, Seq. ID No. 6377, Seq. ID No. 6381, Seq. ID No. 6383, Seq. ID No. 6415, Seq. ID No. 6427, Seq. ID No. 6431, Seq. ID No. 6473, Seq. ID No. 6497, Seq. ID No. 6571, Seq. ID No. 6581, Seq. ID No. 6593, Seq. ID No. 6599, Seq. ID No. 6631, and Seq. ID No. 6639.

31. An isolated polynucleotide of an Alloiococcus otitidis genomic sequence, wherein the polynucleotide comprises a nucleotide sequence having at least about 70% identity to a nucleotide sequence contained within SEQ ID NO: 6651, a complement thereof, a degenerate variant thereof, and a fragment thereof.

32. An isolated polypeptide encoded by a polynucleotide comprising a nucleotide sequence having at least about 70% identity to a nucleotide sequence selected from one of odd numbered sequences from SEQ ID NO: 1 to SEQ ID NO: 6649, a degenerate variant thereof, and a fragment thereof.

33. The polypeptide of claim 32, wherein the polypeptide comprises an amino acid sequence having at least about 70% identity to an amino acid sequence chosen from one of the even-numbered sequences from SEQ ID NO: 2 to SEQ ID NO: 6650, a biological equivalent thereof, and a fragment thereof.

34. The polypeptide of claim 32, wherein the polypeptide is a fusion polypeptide.

35. The polypeptide of claim 32, which immunoreacts with seropositive serum of an individual infected with Alloiococcus otitidis.

36. The polypeptide of claim 32, further defined as:
(a) an Alloiococcus otitidis polypeptide predicted by SignalP algorithm analysis as having met all four criteria set out in the SignalP algorithm for a polypeptide having a signal peptide and predicted by HMM Signal P algorithm analysis as having a signal peptide;
(b) an Alloiococcus otitidis polypeptide predicted by SignalP algorithm analysis as having met three of the four criteria set out in the SignalP algorithm for a polypeptide having a signal peptide and predicted by HMM SignalP algorithm analysis as having a signal peptide;
(c) an Alloiococcus otitidis polypeptide predicted by HMM Signal P algorithm analysis as having a signal peptide;
(d) an Alloiococcus otitidis polypeptide predicted by HMM SignalP algorithm analysis as being a non-secretory protein;
(e) an Alloiococcus otitidis polypeptide identified by BlastP analysis;
(f) an Alloiococcus otitidis polypeptide identified by Pfam analysis;
(g) an Alloiococcus otitidis polypeptide identified using proteomics methodology as sharing homology with surface exposed proteins of Streptococcus pneumoniae;
(h) an Alloiococcus otitidis lipoprotein;
(i) an Alloiococcus otitidis polypeptide having a LPXTG motif, wherein the polypeptide is covalently attached to the peptidoglycan layer;
(j) an Alloiococcus otitidis polypeptide, wherein the polypeptide is non-covalently associated with the peptidoglycan layer;
(k) an Alloiococcus otitidis polypeptide having an RGDX motif wherein X is not a proline residue;
(l) an Alloiococcus otitidis polypeptide predicted by BlastP as being involved in capsule biosynthesis and transport;
(m) an Alloiococcus otitidis polypeptide identified by BlastP as being localized within the capsule loci of the Alloiococcus otitidis genome;
(n) an Alloiococcus otitidis polypeptide predicted by BlastP as being associated with sporulation;
(o) an Alloiococcus otitidis polypeptide encoded by unique ORFs identified by BlastP analysis as having a BlastP ‘E Value’ of >e⁻¹⁰;
(p) an Alloiococcus otitidis polypeptide identified by Glimmer™ ORF finder program;
(q) an Alloiococcus otitidis polypeptide identified by GeneMark™ ORF finder program;
(r) an Alloiococcus otitidis polypeptide identified by an ORF finder program that searches for an ATG, GTG or TTG Start codon between a Stop-Stop region; and
(s) an Alloiococcus otitidis polypeptide identified by an ORF finder program that searches for a transmembrane domain between two Stop codon and a Start codon immediately upstream of this transmembrane region.

37. The Alloiococcus otitidis polypeptide of claim 36, wherein the polypeptide is predicted by SignalP algorithm analysis as having met all four criteria set out in the SignalP algorithm for a polypeptide having a signal peptide and predicted by HMM Signal P algorithm analysis as having a signal peptide; and is selected from one of Seq. ID No. 90, Seq. ID No. 128, Seq. ID No. 132, Seq. ID No. 134, Seq. ID No. 162, Seq. ID No. 164, Seq. ID No. 214, Seq. ID No. 216, Seq. ID No. 226, Seq. ID No. 250, Seq. ID No. 252, Seq. ID No. 288, Seq. ID No. 290, Seq. ID No. 370, Seq. ID No. 372, Seq. ID No. 376, Seq. ID No. 378, Seq. ID No. 498, Seq. ID No. 504, Seq. ID No. 506, Seq. ID No. 528, Seq. ID No. 550, Seq. ID No. 576, Seq. ID No. 618, Seq. ID No. 658, Seq. ID No. 660, Seq. ID No. 662, Seq. ID No. 676, Seq. ID No. 716, Seq. ID No. 718, Seq. ID No. 746, Seq. ID No. 748, Seq. ID No. 756, Seq. ID No. 758, Seq. ID No. 760, Seq. ID No. 762, Seq. ID No. 774, Seq. ID No. 808, Seq. ID No. 968, Seq. ID No. 974, Seq. ID No. 976, Seq. ID No. 1014, Seq. ID No. 1026, Seq. ID No. 1028, Seq. ID No. 1040, Seq. ID No. 1076, Seq. ID No. 1090, Seq. ID No. 1196, Seq. ID No. 1198, Seq. ID No. 1208, Seq. ID No. 1240, Seq. ID No. 1244, Seq. ID No. 1246, Seq. ID No. 1270, Seq. ID No. 1316, Seq. ID No. 1318, Seq. ID No. 1332, Seq. ID No. 1366, Seq. ID No. 1368, Seq. ID No. 1370, Seq. ID No. 1446, Seq. ID No. 1448, Seq. ID No. 1514, Seq. ID No. 1518, Seq. ID No. 1546, Seq. ID No. 1550, Seq. ID No. 1562, Seq. ID No. 1570, Seq. ID No. 1572, Seq. ID No. 1586, Seq. ID No. 1644, Seq. ID No. 1654, Seq. ID No. 1662, Seq. ID No. 1664, Seq. ID No. 1708, Seq. ID No. 1734, Seq. ID No. 1738, Seq. ID No. 1756, Seq. ID No. 1766, Seq. ID No. 1768, Seq. ID No. 1776, Seq. ID No. 1792, Seq. ID No. 1806, Seq. ID No. 1876, Seq. ID No. 1970, Seq. ID No. 1982, Seq. ID No. 2054, Seq. ID No. 2056, Seq. ID No. 2088, Seq. ID No. 2090, Seq. ID No. 2092, Seq. ID No. 2106, Seq. ID No. 2116, Seq. ID No. 2150, Seq. ID No. 2152, Seq. ID No. 2160, Seq. ID No. 2178, Seq. ID No. 2278, Seq. ID No. 2294, Seq. ID No. 2322, Seq. ID No. 2348, Seq. ID No. 2352, Seq. ID No. 2354, Seq. ID No. 2366, Seq. ID No. 2396, Seq. ID No. 2422, Seq. ID No. 2426, Seq. ID No. 2428, Seq. ID No. 2430, Seq. ID No. 2450, Seq. ID No. 2460, Seq. ID No. 2526, Seq. ID No. 2532, Seq. ID No. 2608, Seq. ID No. 2652, Seq. ID No. 2680, Seq. ID No. 2694, Seq. ID No. 2700, Seq. ID No. 2704, Seq. ID No. 2754, Seq. ID No. 2786, Seq. ID No. 2814, Seq. ID No. 2830, Seq. ID No. 2836, Seq. ID No. 2842, Seq. ID No. 2880, Seq. ID No. 2882, Seq. ID No. 2936, Seq. ID No. 2938, Seq. ID No. 2950,.Seq. ID No. 2966, Seq. ID No. 2968, Seq. ID No. 2974, Seq. ID No. 3008, Seq. ID No. 3010, Seq. ID No. 3012, Seq. ID No. 3030, Seq. ID No. 3032, Seq. ID No. 3110, Seq. ID No. 3112, Seq. ID No. 3220, Seq. ID No. 3224, Seq. ID No. 3274, Seq. ID No. 3298, Seq. ID No. 3332, Seq. ID No. 3336, Seq. ID No. 3342, Seq. ID No. 3350, Seq. ID No. 3354, Seq. ID No. 3356, Seq. ID No. 3378, Seq. ID No. 3392, Seq. ID No. 3426, Seq. ID No. 3450, Seq. ID No. 3494, Seq. ID No. 3574, Seq. ID No. 3612, Seq. ID No. 3620, Seq. ID No. 3690, Seq. ID No. 3754, Seq. ID No. 3756, Seq. ID No. 3766, Seq. ID No. 3782, Seq. ID No. 3784, Seq. ID No. 3852, Seq. ID No. 3904, Seq. ID No. 4006, Seq. ID No. 4008, Seq. ID No. 4028, Seq. ID No. 4074, Seq. ID No. 4138, Seq. ID No. 4142, Seq. ID No. 4144, Seq. ID No. 4146, Seq. ID No. 4226, Seq. ID No. 4344, Seq. ID No. 4386, Seq. ID No. 4400, Seq. ID No. 4470, Seq. ID No. 4484, Seq. ID No. 4494, Seq. ID No. 4496, Seq. ID No. 4534, Seq. ID No. 4700, Seq. ID No. 4710, Seq. ID No. 4712, Seq. ID No. 4736, Seq. ID No. 4828, Seq. ID No. 4872, Seq. ID No. 4874, Seq. ID No. 4880, Seq. ID No. 4922, Seq. ID No. 4982, Seq. ID No. 5116, Seq. ID No. 5268, Seq. ID No. 5342, Seq. ID No. 5388, Seq. ID No. 5466, Seq. ID No. 5468, Seq. ID No. 5480, Seq. ID No. 5498, Seq. ID No. 5514, Seq. ID No. 5544, Seq. ID No. 5546, Seq. ID No. 5568, Seq. ID No. 5588, Seq. ID No. 5590, Seq. ID No. 5592, Seq. ID No. 5612, Seq. ID No. 5622, Seq. ID No. 5642, Seq. ID No. 5682, Seq. ID No. 5720, Seq. ID No. 5772, Seq. ID No. 5784, Seq. ID No. 5896, Seq. ID No. 5900, Seq. ID No. 5902, Seq. ID No. 5928, Seq. ID No. 5930, Seq. ID No. 5940, Seq. ID No. 5988, Seq. ID No. 6014, Seq. ID No. 6088, Seq. ID No. 6090, Seq. ID No. 6092, Seq. ID No. 6094, Seq. ID No. 6096, Seq. ID No. 6104, Seq. ID No. 6170, Seq. ID No. 6186, Seq. ID No. 6188, Seq. ID No. 6196, Seq. ID No. 6198, Seq. ID No. 6204, Seq. ID No. 6206, Seq. ID No. 6222, Seq. ID No. 6224, Seq. ID No. 6230, Seq. ID No. 6292, Seq. ID No. 6416, Seq. ID No. 6418, Seq. ID No. 6474, Seq. ID No. 6508, Seq. ID No. 6544, Seq. ID No. 6596, Seq. ID No. 6598, Seq. ID No. 6600, Seq. ID No. 6602, and Seq. ID No. 6604.

38. The Alloiococcus otitidis polypeptide of claim 36, wherein the polypeptide is predicted by SignalP algorithm analysis as having met three of the four criteria set out in the SignalP algorithm for a polypeptide having a signal peptide and predicted by HMM SignalP algorithm analysis as having a signal peptide; and is selected from one of Seq. ID No. 8, Seq. ID No. 12, Seq. ID No. 24, Seq. ID No. 46, Seq. ID No. 48, Seq. ID No. 54, Seq. ID No. 60, Seq. ID No. 66, Seq. ID No. 68, Seq. ID No. 88, Seq. ID No. 92, Seq. ID No. 140, Seq. ID No. 152, Seq. ID No. 160, Seq. ID No. 166, Seq. ID No. 170, Seq. ID No. 186, Seq. ID No. 212, Seq. ID No. 218, Seq. ID No. 236, Seq. ID No. 238, Seq. ID No. 240, Seq. ID No. 272, Seq. ID No. 286, Seq. ID No. 292, Seq. ID No. 328, Seq. ID No. 350, Seq. ID No. 386, Seq. ID No. 388, Seq. ID No. 408, Seq. ID No. 428, Seq. ID No. 432, Seq. ID No. 456, Seq. ID No. 458, Seq. ID No. 496, Seq. ID No. 500, Seq. ID No. 512, Seq. ID No. 514, Seq. ID No. 516, Seq. ID No. 526, Seq. ID No. 546, Seq. ID No. 548, Seq. ID No. 558, Seq. ID No. 570, Seq. ID No. 590, Seq. ID No. 592, Seq. ID No. 594, Seq. ID No. 596, Seq. ID No. 610, Seq. ID No. 638, Seq. ID No. 640, Seq. ID No. 642, Seq. ID No. 654, Seq. ID No. 670, Seq. ID No. 680, Seq. ID No. 720, Seq. ID No. 724, Seq. ID No. 740, Seq. ID No. 750, Seq. ID No. 752, Seq. ID No. 764, Seq. ID No. 766, Seq. ID No. 770, Seq. ID No. 778, Seq. ID No. 780, Seq. ID No. 782, Seq. ID No. 820, Seq. ID No. 896, Seq. ID No. 904, Seq. ID No. 906, Seq. ID No. 910, Seq. ID No. 936, Seq. ID No. 938, Seq. ID No. 952, Seq. ID No. 956, Seq. ID No. 980, Seq. ID No. 982, Seq. ID No. 984, Seq. ID No. 986, Seq. ID No. 1006, Seq. ID No. 1008, Seq. ID No. 1010, Seq. ID No. 1016, Seq. ID No. 1020, Seq. ID No. 1030, Seq. ID No. 1032, Seq. ID No. 1048, Seq. ID No. 1092, Seq. ID No. 1112, Seq. ID No. 1138, Seq. ID No. 1152, Seq. ID No. 1154, Seq. ID No. 1170, Seq. ID No. 1174, Seq. ID No. 1184, Seq. ID No. 1192, Seq. ID No. 1194, Seq. ID No. 1214, Seq. ID No. 1224, Seq. ID No. 1230, Seq. ID No. 1248, Seq. ID No. 1260, Seq. ID No. 1284, Seq. ID No. 1308, Seq. ID No. 1320, Seq. ID No. 1322, Seq. ID No. 1328, Seq. ID No. 1342, Seq. ID No. 1344, Seq. ID No. 1348, Seq. ID No. 1390, Seq. ID No. 1402, Seq. ID No. 1430, Seq. ID No. 1480, Seq. ID No. 1552, Seq. ID No. 1560, Seq. ID No. 1580, Seq. ID No. 1588, Seq. ID No. 1622, Seq. ID No. 1628, Seq. ID No. 1634, Seq. ID No. 1646, Seq. ID No. 1648, Seq. ID No. 1650, Seq. ID No. 1656, Seq. ID No. 1666, Seq. ID No. 1668, Seq. ID No. 1672, Seq. ID No. 1678, Seq. ID No. 1680, Seq. ID No. 1682, Seq. ID No. 1696, Seq. ID No. 1704, Seq. ID No. 1706, Seq. ID No. 1732, Seq. ID No. 1754, Seq. ID No. 1760, Seq. ID No. 1764, Seq. ID No. 1788, Seq. ID No. 1798, Seq. ID No. 1822, Seq. ID No. 1824, Seq. ID No. 1834, Seq. ID No. 1840, Seq. ID No. 1842, Seq. ID No. 1844, Seq. ID No. 1894, Seq. ID No. 1918, Seq. ID No. 1920, Seq. ID No. 1972, Seq. ID No. 1992, Seq. ID No. 2014, Seq. ID No. 2016, Seq. ID No. 2018, Seq. ID No. 2020, Seq. ID No. 2034, Seq. ID No. 2036, Seq. ID No. 2050, Seq. ID No. 2082, Seq. ID No. 2084, Seq. ID No. 2086, Seq. ID No. 2112, Seq. ID No. 2114, Seq. ID No. 2124, Seq. ID No. 2126, Seq. ID No. 2128, Seq. ID No. 2132, Seq. ID No. 2134, Seq. ID No. 2154, Seq. ID No. 2162, Seq. ID No. 2172, Seq. ID No. 2176, Seq. ID No. 2180, Seq. ID No. 2188, Seq. ID No. 2202, Seq. ID No. 2204, Seq. ID No. 2222, Seq. ID No. 2238, Seq. ID No. 2250, Seq. ID No. 2252, Seq. ID No. 2256, Seq. ID No. 2262, Seq. ID No. 2264, Seq. ID No. 2272, Seq. ID No. 2280, Seq. ID No. 2284, Seq. ID No. 2286, Seq. ID No. 2290, Seq. ID No. 2296, Seq. ID No. 2306, Seq. ID No. 2308, Seq. ID No. 2318, Seq. ID No. 2320, Seq. ID No. 2324, Seq. ID No. 2326, Seq. ID No. 2344, Seq. ID No. 2350, Seq. ID No. 2386, Seq. ID No. 2394, Seq. ID No. 2424, Seq. ID No. 2454, Seq. ID No. 2492, Seq. ID No. 2498, Seq. ID No. 2500, Seq. ID No. 2508, Seq. ID No. 2516, Seq. ID No. 2518, Seq. ID No. 2522, Seq. ID No. 2524, Seq. ID No. 2528, Seq. ID No. 2534, Seq. ID No. 2536, Seq. ID No. 2542, Seq. ID No. 2544, Seq. ID No. 2548, Seq. ID No. 2550, Seq. ID No. 2558, Seq. ID No. 2574, Seq. ID No. 2584, Seq. ID No. 2602, Seq. ID No. 2604, Seq. ID No. 2622, Seq. ID No. 2654, Seq. ID No. 2682, Seq. ID No. 2692, Seq. ID No. 2702, Seq. ID No. 2734, Seq. ID No. 2740, Seq. ID No. 2802, Seq. ID No. 2808, Seq. ID No. 2816, Seq. ID No. 2820, Seq. ID No. 2822, Seq. ID No. 2824, Seq. ID No. 2834, Seq. ID No. 2838, Seq. ID No. 2840, Seq. ID No. 2846, Seq. ID No. 2854, Seq. ID No. 2862, Seq. ID No. 2866, Seq. ID No. 2874, Seq. ID No. 2876, Seq. ID No. 2878, Seq. ID No. 2884, Seq. ID No. 2896, Seq. ID No. 2906, Seq. ID No. 2940, Seq. ID No. 2942, Seq. ID No. 2948, Seq. ID No. 2982, Seq. ID No. 2984, Seq. ID No. 2988, Seq. ID No. 2992, Seq. ID No. 2996, Seq. ID No. 3002, Seq. ID No. 3006, Seq. ID No. 3022, Seq. ID No. 3026, Seq. ID No. 3044, Seq. ID No. 3046, Seq. ID No. 3074, Seq. ID No. 3076, Seq. ID No. 3156, Seq. ID No. 3164, Seq. ID No. 3166, Seq. ID No. 3168, Seq. ID No. 3184, Seq. ID No. 3214, Seq. ID No. 3216, Seq. ID No. 3236, Seq. ID No. 3246, Seq. ID No. 3262, Seq. ID No. 3264, Seq. ID No. 3266, Seq. ID No. 3270, Seq. ID No. 3272, Seq. ID No. 3276, Seq. ID No. 3278, Seq. ID No. 3318, Seq. ID No. 3326, Seq. ID No. 3338, Seq. ID No. 3352, Seq. ID No. 3390, Seq. ID No. 3406, Seq. ID No. 3420, Seq. ID No. 3422, Seq. ID No. 3424, Seq. ID No. 3452, Seq. ID No. 3454, Seq. ID No. 3456, Seq. ID No. 3492, Seq. ID No. 3496, Seq. ID No. 3504, Seq. ID No. 3506, Seq. ID No. 3518, Seq. ID No. 3520, Seq. ID No. 3546, Seq. ID No. 3548, Seq. ID No. 3572, Seq. ID No. 3594, Seq. ID No. 3618, Seq. ID No. 3622, Seq. ID No. 3630, Seq. ID No. 3664, Seq. ID No. 3702, Seq. ID No. 3704, Seq. ID No. 3724, Seq. ID No. 3738, Seq. ID No. 3742, Seq. ID No. 3746, Seq. ID No. 3748, Seq. ID No. 3760, Seq. ID No. 3764, Seq. ID No. 3768, Seq. ID No. 3770, Seq. ID No. 3772, Seq. ID No. 3786, Seq. ID No. 3794, Seq. ID No. 3814, Seq. ID No. 3838, Seq. ID No. 3840, Seq. ID No. 3862, Seq. ID No. 3866, Seq. ID No. 3890, Seq. ID No. 3892, Seq. ID No. 3896, Seq. ID No. 3916, Seq. ID No. 3924, Seq. ID No. 3926, Seq. ID No. 3930, Seq. ID No. 3966, Seq. ID No. 3976, Seq. ID No. 3980, Seq. ID No. 3996, Seq. ID No. 3998, Seq. ID No. 4010, Seq. ID No. 4040, Seq. ID No. 4042, Seq. ID No. 4062, Seq. ID No. 4080, Seq. ID No. 4082, Seq. ID No. 4112, Seq. ID No. 4114, Seq. ID No. 4136, Seq. ID No. 4140, Seq. ID No. 4156, Seq. ID No. 4182, Seq. ID No. 4204, Seq. ID No. 4222, Seq. ID No. 4248, Seq. ID No. 4254, Seq. ID No. 4258, Seq. ID No. 4260, Seq. ID No. 4262, Seq. ID No. 4264, Seq. ID No. 4270, Seq. ID No. 4312, Seq. ID No. 4380, Seq. ID No. 4390, Seq. ID No. 4392, Seq. ID No. 4472, Seq. ID No. 4474, Seq. ID No. 4482, Seq. ID No. 4526, Seq. ID No. 4552, Seq. ID No. 4562, Seq. ID No. 4574, Seq. ID No. 4618, Seq. ID No. 4640, Seq. ID No. 4660, Seq. ID No. 4682, Seq. ID No. 4702, Seq. ID No. 4706, Seq. ID No. 4714, Seq. ID No. 4718, Seq. ID No. 4722, Seq. ID No. 4738, Seq. ID No. 4742, Seq. ID No. 4814, Seq. ID No. 4818, Seq. ID No. 4820, Seq. ID No. 4830, Seq. ID No. 4832, Seq. ID No. 4852, Seq. ID No. 4870, Seq. ID No. 4876, Seq. ID No. 4914, Seq. ID No. 4920, Seq. ID No. 4930, Seq. ID No. 4932, Seq. ID No. 4984, Seq. ID No. 4994, Seq. ID No. 5012, Seq. ID No. 5024, Seq. ID No. 5032, Seq. ID No. 5034, Seq. ID No. 5036, Seq. ID No. 5044, Seq. ID No. 5062, Seq. ID No. 5064, Seq. ID No. 5070, Seq. ID No. 5104, Seq. ID No. 5122, Seq. ID No. 5124, Seq. ID No. 5132, Seq. ID No. 5134, Seq. ID No. 5136, Seq. ID No. 5138, Seq. ID No. 5140, Seq. ID No. 5142, Seq. ID No. 5152, Seq. ID No. 5160, Seq. ID No. 5162, Seq. ID No. 5166, Seq. ID No. 5168, Seq. ID No. 5176, Seq. ID No. 5178, Seq. ID No. 5206, Seq. ID No. 5208, Seq. ID No. 5212, Seq. ID No. 5216, Seq. ID No. 5220, Seq. ID No. 5224, Seq. ID No. 5286, Seq. ID No. 5288, Seq. ID No. 5330, Seq. ID No. 5332, Seq. ID No. 5378, Seq. ID No. 5380, Seq. ID No. 5382, Seq. ID No. 5386, Seq. ID No. 5406, Seq. ID No. 5408, Seq. ID No. 5454, Seq. ID No. 5460, Seq. ID No. 5462, Seq. ID No. 5464, Seq. ID No. 5482, Seq. ID No. 5510, Seq. ID No. 5530, Seq. ID No. 5532, Seq. ID No. 5558, Seq. ID No. 5570, Seq. ID No. 5578, Seq. ID No. 5624, Seq. ID No. 5626, Seq. ID No. 5644, Seq. ID No. 5660, Seq. ID No. 5662, Seq. ID No. 5684, Seq. ID No. 5686, Seq. ID No. 5710, Seq. ID No. 5712, Seq. ID No. 5734, Seq. ID No. 5740, Seq. ID No. 5766, Seq. ID No. 5798, Seq. ID No. 5828, Seq. ID No. 5890, Seq. ID No. 5894, Seq. ID No. 5906, Seq. ID No. 5910, Seq. ID No. 5912, Seq. ID No. 5924, Seq. ID No. 5964, Seq. ID No. 5966, Seq. ID No. 6026, Seq. ID No. 6028, Seq. ID No. 6032, Seq. ID No. 6044, Seq. ID No. 6046, Seq. ID No. 6048, Seq. ID No. 6054, Seq. ID No. 6060, Seq. ID No. 6110, Seq. ID No. 6112, Seq. ID No. 6116, Seq. ID No. 6118, Seq. ID No. 6128, Seq. ID No. 6132, Seq. ID No. 6136, Seq. ID No. 6138, Seq. ID No. 6162, Seq. ID No. 6166, Seq. ID No. 6184, Seq. ID No. 6190, Seq. ID No. 6192, Seq. ID No. 6194, Seq. ID No. 6208, Seq. ID No. 6210, Seq. ID No. 6218, Seq. ID No. 6226, Seq. ID No. 6236, Seq. ID No. 6244, Seq. ID No. 6250, Seq. ID No. 6254, Seq. ID No. 6260, Seq. ID No. 6266, Seq. ID No. 6268, Seq. ID No. 6276, Seq. ID No. 6284, Seq. ID No. 6286, Seq. ID No. 6304, Seq. ID No. 6326, Seq. ID No. 6328, Seq. ID No. 6364, Seq. ID No. 6366, Seq. ID No. 6368, Seq. ID No. 6386, Seq. ID No. 6426, Seq. ID No. 6428, Seq. ID No. 6430, Seq. ID No. 6432, Seq. ID No. 6454, Seq. ID No. 6466, Seq. ID No. 6470, Seq. ID No. 6472, Seq. ID No. 6480, Seq. ID No. 6540, Seq. ID No. 6542, Seq. ID No. 6572, Seq. ID No. 6574, Seq. ID No. 6640, Seq. ID No. 6644, and Seq. ID No. 6646.

39. The Alloiococcus otitidis polypeptide of claim 36, wherein the polypeptide is predicted by HMM SignalP algorithm analysis as having a signal peptide, and is selected from one of Seq. ID No. 4, Seq. ID No. 8, Seq. ID No. 10, Seq. ID No. 20, Seq. ID No. 22, Seq. ID No. 32, Seq. ID No. 34, Seq. ID No. 46, Seq. ID No. 48, Seq. ID No. 50, Seq. ID No. 54, Seq. ID No. 66, Seq. ID No. 68, Seq. ID No. 86, Seq. ID No. 88, Seq. ID No. 106, Seq. ID No. 112, Seq. ID No. 124, Seq. ID No. 132, Seq. ID No. 134, Seq. ID No. 142, Seq. ID No. 160, Seq. ID No. 162, Seq. ID No. 164, Seq. ID No. 172, Seq. ID No. 192, Seq. ID No. 210, Seq. ID No. 212, Seq. ID No. 214, Seq. ID No. 216, Seq. ID No. 218, Seq. ID No. 226, Seq. ID No. 236, Seq. ID No. 238, Seq. ID No. 250, Seq. ID No. 252, Seq. ID No. 256, Seq. ID No. 258, Seq. ID No. 260, Seq. ID No. 272, Seq. ID No. 274, Seq. ID No. 278, Seq. ID No. 286, Seq. ID No. 288, Seq. ID No. 290, Seq. ID No. 328, Seq. ID No. 330, Seq. ID No. 358, Seq. ID No. 360, Seq. ID No. 374, Seq. ID No. 376, Seq. ID No. 394, Seq. ID No. 402, Seq. ID No. 404, Seq. ID No. 456, Seq. ID No. 458, Seq. ID No. 478, Seq. ID No. 488, Seq. ID No. 490, Seq. ID No. 492, Seq. ID No. 494, Seq. ID No. 496, Seq. ID No. 498, Seq. ID No. 500, Seq. ID No. 504, Seq. ID No. 506, Seq. ID No. 510, Seq. ID No. 512, Seq. ID No. 514, Seq. ID No. 516, Seq. ID No. 526, Seq. ID No. 528, Seq. ID No. 546, Seq. ID No. 548, Seq. ID No. 550, Seq. ID No. 558, Seq. ID No. 568, Seq. ID No. 570, Seq. ID No. 576, Seq. ID No. 578, Seq. ID No. 580, Seq. ID No. 592, Seq. ID No. 594, Seq. ID No. 600, Seq. ID No. 618, Seq. ID No. 638, Seq. ID No. 640, Seq. ID No. 642, Seq. ID No. 644, Seq. ID No. 646, Seq. ID No. 648, Seq. ID No. 654, Seq. ID No. 656, Seq. ID No. 658, Seq. ID No. 660, Seq. ID No. 662, Seq. ID No. 668, Seq. ID No. 670, Seq. ID No. 676, Seq. ID No. 678, Seq. ID No. 680, Seq. ID No. 716, Seq. ID No. 718, Seq. ID No. 720, Seq. ID No. 722, Seq. ID No. 724, Seq. ID No. 726, Seq. ID No. 746, Seq. ID No. 748, Seq. ID No. 750, Seq. ID No. 752, Seq. ID No. 756, Seq. ID No. 758, Seq. ID No. 760, Seq. ID No. 762, Seq. ID No. 764, Seq. ID No. 766, Seq. ID No. 770, Seq. ID No. 778, Seq. ID No. 780, Seq. ID No. 782, Seq. ID No. 808, Seq. ID No. 810, Seq. ID No. 820, Seq. ID No. 826, Seq. ID No. 864, Seq. ID No. 894, Seq. ID No. 902, Seq. ID No. 936, Seq. ID No. 938, Seq. ID No. 942, Seq. ID No. 944, Seq. ID No. 956, Seq. ID No. 958, Seq. ID No. 968, Seq. ID No. 980, Seq. ID No. 982, Seq. ID No. 984, Seq. ID No. 986, Seq. ID No. 1002, Seq. ID No. 1004, Seq. ID No. 1006, Seq. ID No. 1008, Seq. ID No. 1010, Seq. ID No. 1012, Seq. ID No. 1014, Seq. ID No. 1016, Seq. ID No. 1020, Seq. ID No. 1030, Seq. ID No. 1032, Seq. ID No. 1034, Seq. ID No. 1036, Seq. ID No. 1038, Seq. ID No. 1040, Seq. ID No. 1046, Seq. ID No. 1056, Seq. ID No. 1076, Seq. ID No. 1078, Seq. ID No. 1106, Seq. ID No. 1108, Seq. ID No. 1132, Seq. ID No. 1136, Seq. ID No. 1138, Seq. ID No. 1140, Seq. ID No. 1152, Seq. ID No. 1166, Seq. ID No. 1168, Seq. ID No. 1170, Seq. ID No. 1174, Seq. ID No. 1184, Seq. ID No. 1196, Seq. ID No. 1198, Seq. ID No. 1204, Seq. ID No. 1214, Seq. ID No. 1224, Seq. ID No. 1228, Seq. ID No. 1230, Seq. ID No. 1238, Seq. ID No. 1244, Seq. ID No. 1246, Seq. ID No. 1260, Seq. ID No. 1266, Seq. ID No. 1268, Seq. ID No. 1270, Seq. ID No. 1272, Seq. ID No. 1284, Seq. ID No. 1288, Seq. ID No. 1290, Seq. ID No. 1292, Seq. ID No. 1296, Seq. ID No. 1298, Seq. ID No. 1304, Seq. ID No. 1306, Seq. ID No. 1316, Seq. ID No. 1318, Seq. ID No. 1320, Seq. ID No. 1326, Seq. ID No. 1328, Seq. ID No. 1330, Seq. ID No. 1332, Seq. ID No. 1338, Seq. ID No. 1342, Seq. ID No. 1344, Seq. ID No. 1348, Seq. ID No. 1350, Seq. ID No. 1352, Seq. ID No. 1354, Seq ID No. 1366, Seq. ID No. 1368, Seq. ID No. 1370, Seq. ID No. 1372, Seq. ID No. 1382, Seq. ID No. 1402, Seq. ID No. 1430, Seq. ID No. 1436, Seq. ID No. 1446, Seq. ID No. 1448, Seq. ID No. 1482, Seq. ID No. 1514, Seq. ID No. 1516, Seq. ID No. 1518, Seq. ID No. 1530, Seq. ID No. 1534, Seq. ID No. 1536, Seq. ID No. 1546, Seq. ID No. 1548, Seq. ID No. 1552, Seq. ID No. 1554, Seq. ID No. 1560, Seq. ID No. 1562, Seq. ID No. 1564, Seq. ID No. 1572, Seq. ID No. 1580, Seq. ID No. 1584, Seq. ID No. 1586, Seq. ID No. 1622, Seq. ID No. 1634, Seq. ID No. 1640, Seq. ID No. 1644, Seq. ID No. 1646, Seq. ID No. 1648, Seq. ID No. 1650, Seq. ID No. 1654, Seq. ID No. 1662, Seq. ID No. 1664, Seq. ID No. 1666, Seq. ID No. 1672, Seq. ID No. 1674, Seq. ID No. 1678, Seq. ID No. 1680, Seq. ID No. 1682, Seq. ID No. 1694, Seq. ID No. 1708, Seq. ID No. 1712, Seq. ID No. 1732, Seq. ID No. 1734, Seq. ID No. 1738, Seq. ID No. 1754, Seq. ID No. 1756, Seq. ID No. 1758, Seq. ID No. 1766, Seq. ID No. 1768, Seq. ID No. 1772, Seq. ID No. 1782, Seq. ID No. 1788, Seq. ID No. 1792, Seq. ID No. 1798, Seq. ID No. 1806, Seq. ID No. 1820, Seq. ID No. 1822, Seq. ID No. 1824, Seq. ID No. 1840, Seq. ID No. 1858, Seq. ID No. 1870, Seq. ID No. 1876, Seq. ID No. 1892, Seq. ID No. 1894, Seq. ID No. 1896, Seq. ID No. 1916, Seq. ID No. 1918, Seq. ID No. 1920, Seq. ID No. 1924, Seq. ID No. 1928, Seq. ID No. 1930, Seq. ID No. 1970, Seq. ID No. 1982, Seq. ID No. 1992, Seq. ID No. 1994, Seq. ID No. 2006, Seq. ID No. 2014, Seq. ID No. 2018, Seq. ID No. 2020, Seq. ID No. 2026, Seq. ID No. 2028, Seq. ID No. 2034, Seq. ID No. 2036, Seq. ID No. 2042, Seq. ID No. 2050, Seq. ID No. 2054, Seq. ID No. 2056, Seq. ID No. 2088, Seq. ID No. 2090, Seq. ID No. 2092, Seq. ID No. 2106, Seq. ID No. 2108, Seq. ID No. 2110, Seq. ID No. 2114, Seq. ID No. 2116, Seq. ID No. 2122, Seq. ID No. 2124, Seq. ID No. 2126, Seq. ID No. 2128, Seq. ID No. 2150, Seq. ID No. 2152, Seq. ID No. 2156, Seq. ID No. 2158, Seq. ID No. 2160, Seq. ID No. 2172, Seq. ID No. 2174, Seq. ID No. 2178, Seq. ID No. 2188, Seq. ID No. 2192, Seq. ID No. 2206, Seq. ID No. 2238, Seq. ID No. 2240, Seq. ID No. 2246, Seq. ID No. 2250, Seq. ID No. 2252, Seq. ID No. 2256, Seq. ID No. 2260, Seq. ID No. 2272, Seq. ID No. 2284, Seq. ID No. 2290, Seq. ID No. 2292, Seq. ID No. 2294, Seq. ID No. 2296, Seq. ID No. 2304, Seq. ID No. 2306, Seq. ID No. 2308, Seq. ID No. 2318, Seq. ID No. 2320, Seq. ID No. 2322, Seq. ID No. 2324, Seq. ID No. 2326, Seq. ID No. 2328, Seq. ID No. 2330, Seq. ID No. 2334, Seq. ID No. 2344, Seq. ID No. 2348, Seq. ID No. 2350, Seq. ID No. 2352, Seq. ID No. 2354, Seq. ID No. 2356, Seq. ID No. 2358, Seq. ID No. 2366, Seq. ID No. 2368, Seq. ID No. 2372, Seq. ID No. 2376, Seq. ID No. 2388, Seq. ID No. 2390, Seq. ID No. 2392, Seq. ID No. 2394, Seq. ID No. 2408, Seq. ID No. 2420, Seq. ID No. 2422, Seq. ID No. 2426, Seq. ID No. 2428, Seq. ID No. 2430, Seq. ID No. 2434, Seq. ID No. 2450, Seq. ID No. 2460, Seq. ID No. 2488, Seq. ID No. 2490, Seq. ID No. 2492, Seq. ID No. 2494, Seq. ID No. 2498, Seq. ID No. 2500, Seq. ID No. 2502, Seq. ID No. 2504, Seq. ID No. 2508, Seq. ID No. 2516, Seq. ID No. 2518, Seq. ID No. 2524, Seq. ID No. 2526, Seq. ID No. 2528, Seq. ID No. 2532, Seq. ID No. 2534, Seq. ID No. 2536, Seq. ID No. 2538, Seq. ID No. 2542, Seq. ID No. 2544, Seq. ID No. 2546, Seq. ID No. 2556, Seq. ID No. 2558, Seq. ID No. 2566, Seq. ID No. 2570, Seq. ID No. 2572, Seq. ID No. 2584, Seq. ID No. 2592, Seq. ID No. 2598, Seq. ID No. 2600, Seq. ID No. 2602, Seq. ID No. 2608, Seq. ID No. 2612, Seq. ID No. 2622, Seq. ID No. 2628, Seq. ID No. 2630, Seq. ID No. 2652, Seq. ID No. 2680, Seq. ID No. 2682, Seq. ID No. 2684, Seq. ID No. 2692, Seq. ID No. 2694, Seq. ID No. 2696, Seq. ID No. 2698, Seq. ID No. 2700, Seq. ID No. 2702, Seq. ID No. 2704, Seq. ID No. 2706, Seq. ID No. 2708, Seq. ID No. 2718, Seq. ID No. 2730, Seq. ID No. 2732, Seq. ID No. 2734, Seq. ID No. 2784, Seq. ID No. 2786, Seq. ID No. 2802, Seq. ID No. 2810, Seq. ID No. 2814, Seq. ID No. 2816, Seq. ID No. 2820, Seq. ID No. 2822, Seq. ID No. 2824, Seq. ID No. 2832, Seq. ID No. 2834, Seq. ID No. 2836, Seq. ID No. 2838, Seq. ID No. 2840, Seq. ID No. 2842, Seq. ID No. 2846, Seq. ID No. 2854, Seq. ID No. 2856, Seq. ID No. 2860, Seq. ID No. 2862, Seq. ID No. 2874, Seq. ID No. 2882, Seq. ID No. 2894, Seq. ID No. 2906, Seq. ID No. 2936, Seq. ID No. 2938, Seq. ID No. 2940, Seq. ID No. 2942, Seq. ID No. 2948, Seq. ID No. 2950, Seq. ID No. 2952, Seq. ID No. 2970, Seq. ID No. 2974, Seq. ID No. 2976, Seq. ID No. 2978, Seq. ID No. 2982, Seq. ID No. 2984, Seq. ID No. 2988, Seq. ID No. 2992, Seq. ID No. 2996, Seq. ID No. 2998, Seq. ID No. 3006, Seq. ID No. 3008, Seq. ID No. 3010, Seq. ID No. 3012, Seq. ID No. 3014, Seq. ID No. 3024, Seq. ID No. 3026, Seq. ID No. 3028, Seq. ID No. 3030, Seq. ID No. 3032, Seq. ID No. 3044, Seq. ID No. 3056, Seq. ID No. 3064, Seq. ID No. 3072, Seq. ID No. 3074, Seq. ID No. 3076, Seq. ID No. 3078, Seq. ID No. 3120, Seq. ID No. 3122, Seq. ID No. 3124, Seq. ID No. 3146, Seq. ID No. 3156, Seq. ID No. 3158, Seq. ID No. 3164, Seq. ID No. 3166, Seq. ID No. 3174, Seq. ID No. 3184, Seq. ID No. 3194, Seq. ID No. 3202, Seq. ID No. 3214, Seq. ID No. 3216, Seq. ID No. 3218, Seq. ID No. 3220, Seq. ID No. 3242, Seq. ID No. 3244, Seq. ID No. 3246, Seq. ID No. 3250, Seq. ID No. 3256, Seq. ID No. 3258, Seq. ID No. 3262, Seq. ID No. 3264, Seq. ID No. 3270, Seq. ID No. 3276, Seq. ID No. 3278, Seq. ID No. 3298, Seq. ID No. 3318, Seq. ID No. 3324, Seq. ID No. 3326, Seq. ID No. 3332, Seq. ID No. 3336, Seq. ID No. 3338, Seq. ID No. 3340, Seq. ID No. 3350, Seq. ID No. 3352, Seq. ID No. 3354, Seq. ID No. 3356, Seq. ID No. 3358, Seq. ID No. 3360, Seq. ID No. 3376, Seq. ID No. 3378, Seq. ID No. 3390, Seq. ID No. 3392, Seq. ID No. 3396, Seq. ID No. 3406, Seq. ID No. 3416, Seq. ID No. 3422, Seq. ID No. 3426, Seq. ID No. 3428, Seq. ID No. 3434, Seq. ID No. 3442, Seq. ID No. 3450, Seq. ID No. 3452, Seq. ID No. 3454, Seq. ID No. 3456, Seq. ID No. 3458, Seq. ID No. 3464, Seq. ID No. 3482, Seq. ID No. 3492, Seq. ID No. 3494, Seq. ID No. 3504, Seq. ID No. 3506, Seq. ID No. 3518, Seq. ID No. 3520, Seq. ID No. 3528, Seq. ID No. 3532, Seq. ID No. 3540, Seq. ID No. 3562, Seq. ID No. 3574, Seq. ID No. 3582, Seq. ID No. 3588, Seq. ID No. 3594, Seq. ID No. 3596, Seq. ID No. 3612, Seq. ID No. 3618, Seq. ID No. 3620, Seq. ID No. 3622, Seq. ID No. 3626, Seq. ID No. 3630, Seq. ID No. 3636, Seq. ID No. 3678, Seq. ID No. 3680, Seq. ID No. 3690, Seq. ID No. 3718, Seq. ID No. 3724, Seq. ID No. 3728, Seq. ID No. 3742, Seq. ID No. 3746, Seq. ID No. 3754, Seq. ID No. 3756, Seq. ID No. 3758, Seq. ID No. 3762, Seq. ID No. 3764, Seq. ID No. 3766, Seq. ID No. 3782, Seq. ID No. 3784, Seq. ID No. 3794, Seq. ID No. 3800, Seq. ID No. 3808, Seq. ID No. 3814, Seq. ID No. 3816, Seq. ID No. 3818, Seq. ID No. 3838, Seq. ID No. 3840, Seq. ID No. 3850, Seq. ID No. 3852, Seq. ID No. 3854, Seq. ID No. 3860, Seq. ID No. 3862, Seq. ID No. 3864, Seq. ID No. 3866, Seq. ID No. 3880, Seq. ID No. 3890, Seq. ID No. 3892, Seq. ID No. 3896, Seq. ID No. 3904, Seq. ID No. 3916, Seq. ID No. 3924, Seq. ID No. 3926, Seq. ID No. 3960, Seq. ID No. 3964, Seq. ID No. 3974, Seq. ID No. 3976, Seq. ID No. 3994, Seq. ID No. 3996, Seq. ID No. 3998, Seq. ID No. 4006, Seq. ID No. 4008, Seq. ID No. 4010, Seq. ID No. 4026, Seq. ID No. 4028, Seq. ID No. 4040, Seq. ID No. 4042, Seq. ID No. 4044, Seq. ID No. 4046, Seq. ID No. 4048, Seq. ID No. 4050, Seq. ID No. 4062, Seq. ID No. 4072, Seq. ID No. 4074, Seq. ID No. 4080, Seq. ID No. 4082, Seq. ID No. 4084, Seq. ID No. 4102, Seq. ID No. 4104, Seq. ID No. 4112, Seq. ID No. 4134, Seq. ID No. 4136, Seq. ID No. 4138, Seq. ID No. 4140, Seq. ID No. 4142, Seq. ID No. 4144, Seq. ID No. 4146, Seq. ID No. 4152, Seq. ID No. 4154, Seq. ID No. 4182, Seq. ID No. 4222, Seq. ID No. 4224, Seq. ID No. 4226, Seq. ID No. 4228, Seq. ID No. 4232, Seq. ID No. 4234, Seq. ID No. 4242, Seq. ID No. 4248, Seq. ID No. 4254, Seq. ID No. 4258, Seq. ID No. 4260, Seq. ID No. 4262, Seq. ID No. 4264, Seq. ID No. 4266, Seq. ID No. 4270, Seq. ID No. 4276, Seq. ID No. 4286, Seq. ID No. 4288, Seq. ID No. 4298, Seq. ID No. 4308, Seq. ID No. 4312, Seq. ID No. 4314, Seq. ID No. 4344, Seq. ID No. 4380, Seq. ID No. 4386, Seq. ID No. 4390, Seq. ID No. 4392, Seq. ID No. 4400, Seq. ID No. 4404, Seq. ID No. 4438, Seq. ID No. 4440, Seq. ID No. 4442, Seq. ID No. 4470, Seq. ID No. 4474, Seq. ID No. 4482, Seq. ID No. 4484, Seq. ID No. 4486, Seq. ID No. 4494, Seq. ID No. 4496, Seq. ID No. 4498, Seq. ID No. 4514, Seq. ID No. 4516, Seq. ID No. 4528, Seq. ID No. 4530, Seq. ID No. 4534, Seq. ID No. 4588, Seq. ID No. 4590, Seq. ID No. 4640, Seq. ID No. 4642, Seq. ID No. 4644, Seq. ID No. 4660, Seq. ID No. 4682, Seq. ID No. 4688, Seq. ID No. 4696, Seq. ID No. 4706, Seq. ID No. 4708, Seq. ID No. 4710, Seq. ID No. 4712, Seq. ID No. 4716, Seq. ID No. 4718, Seq. ID No. 4738, Seq. ID No. 4760, Seq. ID No. 4814, Seq. ID No. 4828, Seq. ID No. 4854, Seq. ID No. 4864, Seq. ID No. 4870, Seq. ID No. 4872, Seq. ID No. 4874, Seq. ID No. 4876, Seq. ID No. 4920, Seq. ID No. 4922, Seq. ID No. 4924, Seq. ID No. 4930, Seq. ID No. 4932, Seq. ID No. 4950, Seq. ID No. 4952, Seq. ID No. 4976, Seq. ID No. 4988, Seq. ID No. 5000, Seq. ID No. 5014, Seq. ID No. 5032, Seq. ID No. 5062, Seq. ID No. 5064, Seq. ID No. 5070, Seq. ID No. 5086, Seq. ID No. 5104, Seq. ID No. 5116, Seq. ID No. 5124, Seq. ID No. 5126, Seq. ID No. 5130, Seq. ID No. 5132, Seq. ID No. 5134, Seq. ID No. 5136, Seq. ID No. 5138, Seq. ID No. 5140, Seq. ID No. 5142, Seq. ID No. 5160, Seq. ID No. 5166, Seq. ID No. 5176, Seq. ID No. 5198, Seq. ID No. 5200, Seq. ID No. 5206, Seq. ID No. 5208, Seq. ID No. 5214, Seq. ID No. 5220, Seq. ID No. 5224, Seq. ID No. 5268, Seq. ID No. 5274, Seq. ID No. 5276, Seq. ID No. 5278, Seq. ID No. 5280, Seq. ID No. 5282, Seq. ID No. 5286, Seq. ID No. 5328, Seq. ID No. 5330, Seq. ID No. 5332, Seq. ID No. 5342, Seq. ID No. 5378, Seq. ID No. 5380, Seq. ID No. 5386, Seq. ID No. 5388, Seq. ID No. 5406, Seq. ID No. 5414, Seq. ID No. 5416, Seq. ID No. 5434, Seq. ID No. 5454, Seq. ID No. 5460, Seq. ID No. 5462, Seq. ID No. 5464, Seq. ID No. 5466, Seq. ID No. 5492, Seq. ID No. 5498, Seq. ID No. 5510, Seq. ID No. 5514, Seq. ID No. 5516, Seq. ID No. 5518, Seq. ID No. 5526, Seq. ID No. 5530, Seq. ID No. 5532, Seq. ID No. 5534, Seq. ID No. 5544, Seq. ID No. 5546, Seq. ID No. 5570, Seq. ID No. 5588, Seq. ID No. 5590, Seq. ID No. 5592, Seq. ID No. 5594, Seq. ID No. 5596, Seq. ID No. 5598, Seq. ID No. 5610, Seq. ID No. 5612, Seq. ID No. 5622, Seq. ID No. 5624, Seq. ID No. 5626, Seq. ID No. 5634, Seq. ID No. 5638, Seq. ID No. 5640, Seq. ID No. 5642, Seq. ID No. 5644, Seq. ID No. 5650, Seq. ID No. 5660, Seq. ID No. 5668, Seq. ID No. 5682, Seq. ID No. 5684, Seq. ID No. 5686, Seq. ID No. 5688, Seq. ID No. 5690, Seq. ID No. 5700, Seq. ID No. 5704, Seq. ID No. 5706, Seq. ID No. 5710, Seq. ID No. 5712, Seq. ID No. 5720, Seq. ID No. 5730, Seq. ID No. 5734, Seq. ID No. 5736, Seq. ID No. 5740, Seq. ID No. 5764, Seq. ID No. 5766, Seq. ID No. 5768, Seq. ID No. 5772, Seq. ID No. 5784, Seq. ID No. 5786, Seq. ID No. 5792, Seq. ID No. 5798, Seq. ID No. 5830, Seq. ID No. 5880, Seq. ID No. 5884, Seq. ID No. 5886, Seq. ID No. 5890, Seq. ID No. 5894, Seq. ID No. 5896, Seq. ID No. 5900, Seq. ID No. 5902, Seq. ID No. 5906, Seq. ID No. 5910, Seq. ID No. 5922, Seq. ID No. 5924, Seq. ID No. 5928, Seq. ID No. 5930, Seq. ID No. 5940, Seq. ID No. 6024, Seq. ID No. 6026, Seq. ID No. 6028, Seq. ID No. 6044, Seq. ID No. 6048, Seq. ID No. 6054, Seq. ID No. 6060, Seq. ID No. 6064, Seq. ID No. 6068, Seq. ID No. 6088, Seq. ID No. 6090, Seq. ID No. 6092, Seq. ID No. 6096, Seq. ID No. 6104, Seq. ID No. 6108, Seq. ID No. 6110, Seq. ID No. 6112, Seq. ID No. 6116, Seq. ID No. 6118, Seq. ID No. 6136, Seq. ID No. 6138, Seq. ID No. 6160, Seq. ID No. 6162, Seq. ID No. 6164, Seq. ID No. 6166, Seq. ID No. 6170, Seq. ID No. 6184, Seq. ID No. 6186, Seq. ID No. 6188, Seq. ID No. 6190, Seq. ID No. 6192, Seq. ID No. 6198, Seq. ID No. 6204, Seq. ID No. 6206, Seq. ID No. 6210, Seq. ID No. 6218, Seq. ID No. 6222, Seq. ID No. 6224, Seq. ID No. 6226, Seq. ID No. 6228, Seq. ID No. 6230, Seq. ID No. 6236, Seq. ID No. 6238, Seq. ID No. 6242, Seq. ID No. 6244, Seq. ID No. 6246, Seq. ID No. 6250, Seq. ID No. 6254, Seq. ID No. 6260, Seq. ID No. 6264, Seq. ID No. 6266, Seq. ID No. 6268, Seq. ID No. 6274, Seq. ID No. 6276, Seq. ID No. 6284, Seq. ID No. 6286, Seq. ID No. 6290, Seq. ID No. 6302, Seq. ID No. 6304, Seq. ID No. 6326, Seq. ID No. 6328, Seq. ID No. 6364, Seq. ID No. 6366, Seq. ID No. 6378, Seq. ID No. 6382, Seq. ID No. 6384, Seq. ID No. 6416, Seq. ID No. 6426, Seq. ID No. 6428, Seq. ID No. 6430, Seq. ID No. 6432, Seq. ID No. 6474, Seq. ID No. 6480, Seq. ID No. 6498, Seq. ID No. 6518, Seq. ID No. 6542, Seq. ID No. 6544, Seq. ID No. 6556, Seq. ID No. 6558, Seq. ID No. 6572, Seq. ID No. 6574, Seq. ID No. 6582, Seq. ID No. 6594, Seq. ID No. 6596, Seq. ID No. 6598, Seq. ID No. 6600, Seq. ID No. 6602, Seq. ID No. 6604, Seq. ID No. 6632, Seq. ID No. 6638, Seq. ID No. 6640, Seq. ID No. 6644, Seq. ID No. 6646, and Seq. ID No. 6650.

40. The Alloiococcus otitidis polypeptide of claim 36, wherein the polypeptide is predicted by HMM SignalP algorithm analysis as being a non-secretory protein, and is selected from one of Seq. ID No. 2, Seq. ID No. 6, Seq. ID No. 12, Seq. ID No. 14, Seq. ID No. 16, Seq. ID No. 18, Seq. ID No. 24, Seq. ID No. 26, Seq. ID No. 28, Seq. ID No. 30, Seq. ID No. 36, Seq. ID No. 38, Seq. ID No. 40, Seq. ID No. 42, Seq. ID No. 44, Seq. ID No. 52, Seq. ID No. 56, Seq. ID No. 58, Seq. ID No. 60, Seq. ID No. 62, Seq. ID No. 64, Seq. ID No. 70, Seq. ID No. 72, Seq. ID No. 74, Seq. ID No. 76, Seq. ID No. 78, Seq. ID No. 80, Seq. ID No. 82, Seq. ID No. 84, Seq. ID No. 90, Seq. ID No. 92, Seq. ID No. 94, Seq. ID No. 96, Seq. ID No. 98, Seq. ID No. 100, Seq. ID No. 102, Seq. ID No. 104, Seq. ID No. 108, Seq. ID No. 110, Seq. ID No. 114, Seq. ID No. 116, Seq. ID No. 118, Seq. ID No. 120, Seq. ID No. 122, Seq. ID No. 126, Seq. ID No. 128, Seq. ID No. 130, Seq. ID No. 136, Seq. ID No. 138, Seq. ID No. 140, Seq. ID No. 144, Seq. ID No. 146, Seq. ID No. 148, Seq. ID No. 150, Seq. ID No. 152, Seq. ID No. 154, Seq. ID No. 156, Seq. ID No. 158, Seq. ID No. 166, Seq. ID No. 168, Seq. ID No. 170, Seq. ID No. 174, Seq. ID No. 176, Seq. ID No. 178, Seq. ID No. 180, Seq. ID No. 182, Seq. ID No. 184, Seq. ID No. 186, Seq. ID No. 188, Seq. ID No. 190, Seq. ID No. 194, Seq. ID No. 196, Seq. ID No. 198, Seq. ID No. 200, Seq. ID No. 202, Seq. ID No. 204, Seq. ID No. 206, Seq. ID No. 208, Seq. ID No. 220, Seq. ID No. 222, Seq. ID No. 224, Seq. ID No. 228, Seq. ID No. 230, Seq. ID No. 232, Seq. ID No. 234, Seq. ID No. 240, Seq. ID No. 242, Seq. ID No. 244, Seq. ID No. 246, Seq. ID No. 248, Seq. ID No. 254, Seq. ID No. 262, Seq. ID No. 264, Seq. ID No. 266, Seq. ID No. 268, Seq. ID No. 270, Seq. ID No. 276, Seq. ID No. 280, Seq. ID No. 282, Seq. ID No. 284, Seq. ID No. 292, Seq. ID No. 294, Seq. ID No. 296, Seq. ID No. 298, Seq. ID No. 300, Seq. ID No. 302, Seq. ID No. 304, Seq. ID No. 306, Seq. ID No. 308, Seq. ID No. 310, Seq. ID No. 312, Seq. ID No. 314, Seq. ID No. 316, Seq. ID No. 318, Seq. ID No. 320, Seq. ID No. 322, Seq. ID No. 324, Seq. ID No. 326, Seq. ID No. 332, Seq. ID No. 334, Seq. ID No. 336, Seq. ID No. 338, Seq. ID No. 340, Seq. ID No. 342, Seq. ID No. 344, Seq. ID No. 346, Seq. ID No. 348, Seq. ID No. 350, Seq. ID No. 352, Seq. ID No. 354, Seq. ID No. 356, Seq. ID No. 362, Seq. ID No. 364, Seq. ID No. 366, Seq. ID No. 368, Seq. ID No. 370, Seq. ID No. 372, Seq. ID No. 378, Seq. ID No. 380, Seq. ID No. 382, Seq. ID No. 384, Seq. ID No. 386, Seq. ID No. 388, Seq. ID No. 390, Seq. ID No. 392, Seq. ID No. 396, Seq. ID No. 398, Seq. ID No. 400, Seq. ID No. 406, Seq. ID No. 408, Seq. ID No. 410, Seq. ID No. 412, Seq. ID No. 414, Seq. ID No. 416, Seq. ID No. 418, Seq. ID No. 420, Seq. ID No. 422, Seq. ID No. 424, Seq. ID No. 426, Seq. ID No. 428, Seq. ID No. 430, Seq. ID No. 432, Seq. ID No. 434, Seq. ID No. 436, Seq. ID No. 438, Seq. ID No. 440, Seq. ID No. 442, Seq. ID No. 444, Seq. ID No. 446, Seq. ID No. 448, Seq. ID No. 450, Seq. ID No. 452, Seq. ID No. 454, Seq. ID No. 460, Seq. ID No. 462, Seq. ID No. 464, Seq. ID No. 466, Seq. ID No. 468, Seq. ID No. 470, Seq. ID No. 472, Seq. ID No. 474, Seq. ID No. 476, Seq. ID No. 480, Seq. ID No. 482, Seq. ID No. 484, Seq. ID No. 486, Seq. ID No. 502, Seq. ID No. 508, Seq. ID No. 518, Seq. ID No. 520, Seq. ID No. 522, Seq. ID No. 524, Seq. ID No. 530, Seq. ID No. 532, Seq. ID No. 534, Seq. ID No. 536, Seq. ID No. 538, Seq. ID No. 540, Seq. ID No. 542, Seq. ID No. 544, Seq. ID No. 552, Seq. ID No. 554, Seq. ID No. 556, Seq. ID No. 560, Seq. ID No. 562, Seq. ID No. 564, Seq. ID No. 566, Seq. ID No. 572, Seq. ID No. 574, Seq. ID No. 582, Seq. ID No. 584, Seq. ID No. 586, Seq. ID No. 588, Seq. ID No. 590, Seq. ID No. 596, Seq. ID No. 598, Seq. ID No. 602, Seq. ID No. 604, Seq. ID No. 606, Seq. ID No. 608, Seq. ID No. 610, Seq. ID No. 612, Seq. ID No. 614, Seq. ID No. 616, Seq. ID No. 620, Seq. ID No. 622, Seq. ID No. 624, Seq. ID No. 626, Seq. ID No. 628, Seq. ID No. 630, Seq. ID No. 632, Seq. ID No. 634, Seq. ID No. 636, Seq. ID No. 650, Seq. ID No. 652, Seq. ID No. 664, Seq. ID No. 666, Seq. ID No. 672, Seq. ID No. 674, Seq. ID No. 682, Seq. ID No. 684, Seq. ID No. 686, Seq. ID No. 688, Seq. ID No. 690, Seq. ID No. 692, Seq. ID No. 694, Seq. ID No. 696, Seq. ID No. 698, Seq. ID No. 700, Seq. ID No. 702, Seq. ID No. 704, Seq. ID No. 706, Seq. ID No. 708, Seq. ID No. 710, Seq. ID No. 712, Seq. ID No. 714, Seq. ID No. 728, Seq. ID No. 730, Seq. ID No. 732, Seq. ID No. 734, Seq. ID No. 736, Seq. ID No. 738, Seq. ID No. 740, Seq. ID No. 742, Seq. ID No. 744, Seq. ID No. 754, Seq. ID No. 768, Seq. ID No. 772, Seq. ID No. 774, Seq. ID No. 776, Seq. ID No. 784, Seq. ID No. 786, Seq. ID No. 788, Seq. ID No. 790, Seq. ID No. 792, Seq. ID No. 794, Seq. ID No. 796, Seq. ID No. 798, Seq. ID No. 800, Seq. ID No. 802, Seq. ID No. 804, Seq. ID No. 806, Seq. ID No. 812, Seq. ID No. 814, Seq. ID No. 816, Seq. ID No. 818, Seq. ID No. 822, Seq. ID No. 824, Seq. ID No. 828, Seq. ID No. 830, Seq. ID No. 832, Seq. ID No. 834, Seq. ID No. 836, Seq. ID No. 838, Seq. ID No. 840, Seq. ID No. 842, Seq. ID No. 844, Seq. ID No. 846, Seq. ID No. 848, Seq. ID No. 850, Seq. ID No. 852, Seq. ID No. 854, Seq. ID No. 856, Seq. ID No. 858, Seq. ID No. 860, Seq. ID No. 862, Seq. ID No. 866, Seq. ID No. 868, Seq. ID No. 870, Seq. ID No. 872, Seq. ID No. 874, Seq. ID No. 876, Seq. ID No. 878, Seq. ID No. 880, Seq. ID No. 882, Seq. ID No. 884, Seq. ID No. 886, Seq. ID No. 888, Seq. ID No. 890, Seq. ID No. 892, Seq. ID No. 896, Seq. ID No. 898, Seq. ID No. 900, Seq. ID No. 904, Seq. ID No. 906, Seq. ID No. 908, Seq. ID No. 910, Seq. ID No. 912, Seq. ID No. 914, Seq. ID No. 916, Seq. ID No. 918, Seq. ID No. 920, Seq. ID No. 922, Seq. ID No. 924, Seq. ID No. 926, Seq. ID No. 928, Seq. ID No. 930, Seq. ID No. 932, Seq. ID No. 934, Seq. ID No. 940, Seq. ID No. 946, Seq. ID No. 948, Seq. ID No. 950, Seq. ID No. 952, Seq. ID No. 954, Seq. ID No. 960, Seq. ID No. 962, Seq. ID No. 964, Seq. ID No. 966, Seq. ID No. 970, Seq. ID No. 972, Seq. ID No. 974, Seq. ID No. 976, Seq. ID No. 978, Seq. ID No. 988, Seq. ID No. 990, Seq. ID No. 992, Seq. ID No. 994, Seq. ID No. 996, Seq. ID No. 998, Seq. ID No. 1000, Seq. ID No. 1018, Seq. ID No. 1022, Seq. ID No. 1024, Seq. ID No. 1026, Seq. ID No. 1028, Seq. ID No. 1042, Seq. ID No. 1044, Seq. ID No. 1048, Seq. ID No. 1050, Seq. ID No. 1052, Seq. ID No. 1054, Seq. ID No. 1058, Seq. ID No. 1060, Seq. ID No. 1062, Seq. ID No. 1064, Seq. ID No. 1066, Seq. ID No. 1068, Seq. ID No. 1070, Seq. ID No. 1072, Seq. ID No. 1074, Seq. ID No. 1080, Seq. ID No. 1082, Seq. ID No. 1084, Seq. ID No. 1086, Seq. ID No. 1088, Seq. ID No. 1090, Seq. ID No. 1092, Seq. ID No. 1094, Seq. ID No. 1096, Seq. ID No. 1098, Seq. ID No. 1100, Seq. ID No. 1102, Seq. ID No. 1104, Seq. ID No. 1110, Seq. ID No. 1112, Seq. ID No. 1114, Seq. ID No. 1116, Seq. ID No. 1118, Seq. ID No. 1120, Seq. ID No. 1122, Seq. ID No. 1124, Seq. ID No. 1126, Seq. ID No. 1128, Seq. ID No. 1130, Seq. ID No. 1134, Seq. ID No. 1142, Seq. ID No. 1144, Seq. ID No. 1146, Seq. ID No. 1148, Seq. ID No. 1150, Seq. ID No. 1154, Seq. ID No. 1156, Seq. ID No. 1158, Seq. ID No. 1160, Seq. ID No. 1162, Seq. ID No. 1164, Seq. ID No. 1172, Seq. ID No. 1176, Seq. ID No. 1178, Seq. ID No. 1180, Seq. ID No. 1182, Seq. ID No. 1186, Seq. ID No. 1188, Seq. ID No. 1190, Seq. ID No. 1192, Seq. ID No. 1194, Seq. ID No. 1200, Seq. ID No. 1202, Seq. ID No. 1206, Seq. ID No. 1208, Seq. ID No. 1210, Seq. ID No. 1212, Seq. ID No. 1216, Seq. ID No. 1218, Seq. ID No. 1220, Seq. ID No. 1222, Seq. ID No. 1226, Seq. ID No. 1232, Seq. ID No. 1234, Seq. ID No. 1236, Seq. ID No. 1240, Seq. ID No. 1242, Seq. ID No. 1248, Seq. ID No. 1250, Seq. ID No. 1252, Seq. ID No. 1254, Seq. ID No. 1256, Seq. ID No. 1258, Seq. ID No. 1262, Seq. ID No. 1264, Seq. ID No. 1274, Seq. ID No. 1276, Seq. ID No. 1278, Seq. ID No. 1280, Seq. ID No. 1282, Seq. ID No. 1286, Seq. ID No. 1294, Seq. ID No. 1300, Seq. ID No. 1302, Seq. ID No. 1308, Seq. ID No. 1310, Seq. ID No. 1312, Seq. ID No. 1314, Seq. ID No. 1322, Seq. ID No. 1324, Seq. ID No. 1334, Seq. ID No. 1336, Seq. ID No. 1340, Seq. ID No. 1346, Seq. ID No. 1356, Seq. ID No. 1358, Seq. ID No. 1360, Seq. ID No. 1362, Seq. ID No. 1364, Seq. ID No. 1374, Seq. ID No. 1376, Seq. ID No. 1378, Seq. ID No. 1380, Seq. ID No. 1384, Seq. ID No. 1386, Seq. ID No. 1388, Seq. ID No. 1390, Seq. ID No. 1392, Seq. ID No. 1394, Seq. ID No. 1396, Seq. ID No. 1398, Seq. ID No. 1400, Seq. ID No. 1404, Seq. ID No. 1406, Seq. ID No. 1408, Seq. ID No. 1410, Seq. ID No. 1412, Seq. ID No. 1414, Seq. ID No. 1416, Seq. ID No. 1418, Seq. ID No. 1420, Seq. ID No. 1422, Seq. ID No. 1424, Seq. ID No. 1426, Seq. ID No. 1428, Seq. ID No. 1432, Seq. ID No. 1434, Seq. ID No. 1438, Seq. ID No. 1440, Seq. ID No. 1442, Seq. ID No. 1444, Seq. ID No. 1450, Seq. ID No. 1452, Seq. ID No. 1454, Seq. ID No. 1456, Seq. ID No. 1458, Seq. ID No. 1460, Seq. ID No. 1462, Seq. ID No. 1464, Seq. ID No. 1466, Seq. ID No. 1468, Seq. ID No. 1470, Seq. ID No. 1472, Seq. ID No. 1474, Seq. ID No. 1476, Seq. ID No. 1478, Seq. ID No. 1480, Seq. ID No. 1484, Seq. ID No. 1486, Seq. ID No. 1488, Seq. ID No. 1490, Seq. ID No. 1492, Seq. ID No. 1494, Seq. ID No. 1496, Seq. ID No. 1498, Seq. ID No. 1500, Seq. ID No. 1502, Seq. ID No. 1504, Seq. ID No. 1506, Seq. ID No. 1508, Seq. ID No. 1510, Seq. ID No. 1512, Seq. ID No. 1520, Seq. ID No. 1522, Seq. ID No. 1524, Seq. ID No. 1526, Seq. ID No. 1528, Seq. ID No. 1532, Seq. ID No. 1538, Seq. ID No. 1540, Seq. ID No. 1542, Seq. ID No. 1544, Seq. ID No. 1550, Seq. ID No. 1556, Seq. ID No. 1558, Seq. ID No. 1566, Seq. ID No. 1568, Seq. ID No. 1570, Seq. ID No. 1574, Seq. ID No. 1576, Seq. ID No. 1578, Seq. ID No. 1582, Seq. ID No. 1588, Seq. ID No. 1590, Seq. ID No. 1592, Seq. ID No. 1594, Seq. ID No. 1596, Seq. ID No. 1598, Seq. ID No. 1602, Seq. ID No. 1604, Seq. ID No. 1606, Seq. ID No. 1608, Seq. ID No. 1610, Seq. ID No. 1612, Seq. ID No. 1614, Seq. ID No. 1616, Seq. ID No. 1618, Seq. ID No. 1620, Seq. ID No. 1624, Seq. ID No. 1626, Seq. ID No. 1628, Seq. ID No. 1630, Seq. ID No. 1632, Seq. ID No. 1636, Seq. ID No. 1638, Seq. ID No. 1642, Seq. ID No. 1652, Seq. ID No. 1656, Seq. ID No. 1658, Seq. ID No. 1660, Seq. ID No. 1668, Seq. ID No. 1670, Seq. ID No. 1676, Seq. ID No. 1684, Seq. ID No. 1686, Seq. ID No. 1688, Seq. ID No. 1690, Seq. ID No. 1692, Seq. ID No. 1696, Seq. ID No. 1698, Seq. ID No. 1700, Seq. ID No. 1702, Seq. ID No. 1704, Seq. ID No. 1706, Seq. ID No. 1710, Seq. ID No. 1714, Seq. ID No. 1716, Seq. ID No. 1718, Seq. ID No. 1720, Seq. ID No. 1722, Seq. ID No. 1724, Seq. ID No. 1726, Seq. ID No. 1728, Seq. ID No. 1730, Seq. ID No. 1736, Seq. ID No. 1740, Seq. ID No. 1742, Seq. ID No. 1744, Seq. ID No. 1746, Seq. ID No. 1748, Seq. ID No. 1750, Seq. ID No. 1752, Seq. ID No. 1760, Seq. ID No. 1762, Seq. ID No. 1764, Seq. ID No. 1770, Seq. ID No. 1774, Seq. ID No. 1776, Seq. ID No. 1778, Seq. ID No. 1780, Seq. ID No. 1784, Seq. ID No. 1786, Seq. ID No. 1790, Seq. ID No. 1794, Seq. ID No. 1796, Seq. ID No. 1800, Seq. ID No. 1802, Seq. ID No. 1804, Seq. ID No. 1808, Seq. ID No. 1810, Seq. ID No. 1812, Seq. ID No. 1814, Seq. ID No. 1816, Seq. ID No. 1818, Seq. ID No. 1826, Seq. ID No. 1828, Seq. ID No. 1830, Seq. ID No. 1832, Seq. ID No. 1834, Seq. ID No. 1836, Seq. ID No. 1838, Seq. ID No. 1842, Seq. ID No. 1844, Seq. ID No. 1846, Seq. ID No. 1848, Seq. ID No. 1850, Seq. ID No. 1852, Seq. ID No. 1854, Seq. ID No. 1856, Seq. ID No. 1860, Seq. ID No. 1862, Seq. ID No. 1864, Seq. ID No. 1866, Seq. ID No. 1868, Seq. ID No. 1872, Seq. ID No. 1874, Seq. ID No. 1878, Seq. ID No. 1880, Seq. ID No. 1882, Seq. ID No. 1884, Seq. ID No. 1886, Seq. ID No. 1888, Seq. ID No. 1890, Seq. ID No. 1898, Seq. ID No. 1900, Seq. ID No. 1902, Seq. ID No. 1904, Seq. ID No. 1906, Seq. ID No. 1908, Seq. ID No. 1910, Seq. ID No. 1912, Seq. ID No. 1914, Seq. ID No. 1922, Seq. ID No. 1926, Seq. ID No. 1932, Seq. ID No. 1934, Seq. ID No. 1936, Seq. ID No. 1938, Seq. ID No. 1940, Seq. ID No. 1942, Seq. ID No. 1944, Seq. ID No. 1946, Seq. ID No. 1948, Seq. ID No. 1950, Seq. ID No. 1952, Seq. ID No. 1954, Seq. ID No. 1956, Seq. ID No. 1958, Seq. ID No. 1960, Seq. ID No. 1962, Seq. ID No. 1964, Seq. ID No. 1966, Seq. ID No. 1968, Seq. ID No. 1972, Seq. ID No. 1974, Seq. ID No. 1976, Seq. ID No. 1978, Seq. ID No. 1980, Seq. ID No. 1984, Seq. ID No. 1986, Seq. ID No. 1988, Seq. ID No. 1990, Seq. ID No. 1996, Seq. ID No. 1998, Seq. ID No. 2000, Seq. ID No. 2002, Seq. ID No. 2004, Seq. ID No. 2008, Seq. ID No. 2010, Seq. ID No. 2012, Seq. ID No. 2016, Seq. ID No. 2022, Seq. ID No. 2024, Seq. ID No. 2030, Seq. ID No. 2032, Seq. ID No. 2038, Seq. ID No. 2040, Seq. ID No. 2044, Seq. ID No. 2046, Seq. ID No. 2048, Seq. ID No. 2052, Seq. ID No. 2058, Seq. ID No. 2060, Seq. ID No. 2062, Seq. ID No. 2064, Seq. ID No. 2066, Seq. ID No. 2068, Seq. ID No. 2070, Seq. ID No. 2072, Seq. ID No. 2074, Seq. ID No. 2076, Seq. ID No. 2078, Seq. ID No. 2080, Seq. ID No. 2082, Seq. ID No. 2084, Seq. ID No. 2086, Seq. ID No. 2094, Seq. ID No. 2096, Seq. ID No. 2098, Seq. ID No. 2100, Seq. ID No. 2102, Seq. ID No. 2104, Seq. ID No. 2112, Seq. ID No. 2118, Seq. ID No. 2120, Seq. ID No. 2130, Seq. ID No. 2132, Seq. ID No. 2134, Seq. ID No. 2136, Seq. ID No. 2138, Seq. ID No. 2140, Seq. ID No. 2142, Seq. ID No. 2144, Seq. ID No. 2146, Seq. ID No. 2148, Seq. ID No. 2154, Seq. ID No. 2162, Seq. ID No. 2164, Seq. ID No. 2166, Seq. ID No. 2168, Seq. ID No. 2170, Seq. ID No. 2176, Seq. ID No. 2180, Seq. ID No. 2182, Seq. ID No. 2184, Seq. ID No. 2186, Seq. ID No. 2190, Seq. ID No. 2194, Seq. ID No. 2196, Seq. ID No. 2198, Seq. ID No. 2200, Seq. ID No. 2202, Seq. ID No. 2204, Seq. ID No. 2208, Seq. ID No. 2210, Seq. ID No. 2212, Seq. ID No. 2214, Seq. ID No. 2216, Seq. ID No. 2218, Seq. ID No. 2220, Seq. ID No. 2222, Seq. ID No. 2224, Seq. ID No. 2226, Seq. ID No. 2228, Seq. ID No. 2230, Seq. ID No. 2232, Seq. ID No. 2234, Seq. ID No. 2236, Seq. ID No. 2242, Seq. ID No. 2244, Seq. ID No. 2248, Seq. ID No. 2254, Seq. ID No. 2258, Seq. ID No. 2262, Seq. ID No. 2264, Seq. ID No. 2266, Seq. ID No. 2268, Seq. ID No. 2270, Seq. ID No. 2274, Seq. ID No. 2276, Seq. ID No. 2278, Seq. ID No. 2280, Seq. ID No. 2282, Seq. ID No. 2286, Seq. ID No. 2288, Seq. ID No. 2298, Seq. ID No. 2300, Seq. ID No. 2302, Seq. ID No. 2310, Seq. ID No. 2312, Seq. ID No. 2314, Seq. ID No. 2316, Seq. ID No. 2332, Seq. ID No. 2336, Seq. ID No. 2338, Seq. ID No. 2340, Seq. ID No. 2342, Seq. ID No. 2346, Seq. ID No. 2360, Seq. ID No. 2362, Seq. ID No. 2364, Seq. ID No. 2370, Seq. ID No. 2374, Seq. ID No. 2378, Seq. ID No. 2380, Seq. ID No. 2382, Seq. ID No. 2384, Seq. ID No. 2386, Seq. ID No. 2396, Seq. ID No. 2398, Seq. ID No. 2400, Seq. ID No. 2402, Seq. ID No. 2404, Seq. ID No. 2406, Seq. ID No. 2410, Seq. ID No. 2412, Seq. ID No. 2414, Seq. ID No. 2416, Seq. ID No. 2418, Seq. ID No. 2424, Seq. ID No. 2432, Seq. ID No. 2436, Seq. ID No. 2438, Seq. ID No. 2440, Seq. ID No. 2442, Seq. ID No. 2444, Seq. ID No. 2446, Seq. ID No. 2448, Seq. ID No. 2452, Seq. ID No. 2454, Seq. ID No. 2456, Seq. ID No. 2458, Seq. ID No. 2462, Seq. ID No. 2464, Seq. ID No. 2466, Seq. ID No. 2468, Seq. ID No. 2470, Seq. ID No. 2472, Seq. ID No. 2474, Seq. ID No. 2476, Seq. ID No. 2478, Seq. ID No. 2480, Seq. ID No. 2482, Seq. ID No. 2484, Seq. ID No. 2486, Seq. ID No. 2496, Seq. ID No. 2506, Seq. ID No. 2510, Seq. ID No. 2512, Seq. ID No. 2514, Seq. ID No. 2520, Seq. ID No. 2522, Seq. ID No. 2530, Seq. ID No. 2540, Seq. ID No. 2548, Seq. ID No. 2550, Seq. ID No. 2552, Seq. ID No. 2554, Seq. ID No. 2560, Seq. ID No. 2562, Seq. ID No. 2564, Seq. ID No. 2568, Seq. ID No. 2574, Seq. ID No. 2576, Seq. ID No. 2578, Seq. ID No. 2580, Seq. ID No. 2582, Seq. ID No. 2586, Seq. ID No. 2588, Seq. ID No. 2590, Seq. ID No. 2594, Seq. ID No. 2596, Seq. ID No. 2604, Seq. ID No. 2606, Seq. ID No. 2610, Seq. ID No. 2614, Seq. ID No. 2616, Seq. ID No. 2618, Seq. ID No. 2620, Seq. ID No. 2624, Seq. ID No. 2626, Seq. ID No. 2632, Seq. ID No. 2634, Seq. ID No. 2636, Seq. ID No. 2638, Seq. ID No. 2640, Seq. ID No. 2642, Seq. ID No. 2644, Seq. ID No. 2646, Seq. ID No. 2648, Seq. ID No. 2650, Seq. ID No. 2654, Seq. ID No. 2656, Seq. ID No. 2658, Seq. ID No. 2660, Seq. ID No. 2662, Seq. ID No. 2664, Seq. ID No. 2666, Seq. ID No. 2668, Seq. ID No. 2670, Seq. ID No. 2672, Seq. ID No. 2674, Seq. ID No. 2676, Seq. ID No. 2678, Seq. ID No. 2686, Seq. ID No. 2688, Seq. ID No. 2690, Seq. ID No. 2710, Seq. ID No. 2712, Seq. ID No. 2714, Seq. ID No. 2716, Seq. ID No. 2720, Seq. ID No. 2722, Seq. ID No. 2724, Seq. ID No. 2726, Seq. ID No. 2728, Seq. ID No. 2736, Seq. ID No. 2738, Seq. ID No. 2740, Seq. ID No. 2742, Seq. ID No. 2744, Seq. ID No. 2746, Seq. ID No. 2748, Seq. ID No. 2750, Seq. ID No. 2752, Seq. ID No. 2754, Seq. ID No. 2756, Seq. ID No. 2758, Seq. ID No. 2760, Seq. ID No. 2762, Seq. ID No. 2764, Seq. ID No. 2766, Seq. ID No. 2768, Seq. ID No. 2770, Seq. ID No. 2772, Seq. ID No. 2774, Seq. ID No. 2776, Seq. ID No. 2778, Seq. ID No. 2780, Seq. ID No. 2782, Seq. ID No. 2788, Seq. ID No. 2790, Seq. ID No. 2792, Seq. ID No. 2794, Seq. ID No. 2796, Seq. ID No. 2798, Seq. ID No. 2800, Seq. ID No. 2804, Seq. ID No. 2806, Seq. ID No. 2808, Seq. ID No. 2812, Seq. ID No. 2818, Seq. ID No. 2826, Seq. ID No. 2828, Seq. ID No. 2830, Seq. ID No. 2844, Seq. ID No. 2848, Seq. ID No. 2850, Seq. ID No. 2852, Seq. ID No. 2858, Seq. ID No. 2864, Seq. ID No. 2866, Seq. ID No. 2868, Seq. ID No. 2870, Seq. ID No. 2872, Seq. ID No. 2876, Seq. ID No. 2878, Seq. ID No. 2880, Seq. ID No. 2884, Seq. ID No. 2886, Seq. ID No. 2888, Seq. ID No. 2890, Seq. ID No. 2892, Seq. ID No. 2896, Seq. ID No. 2898, Seq. ID No. 2900, Seq. ID No. 2902, Seq. ID No. 2904, Seq. ID No. 2908, Seq. ID No. 2910, Seq. ID No. 2912, Seq. ID No. 2914, Seq. ID No. 2916, Seq. ID No. 2918, Seq. ID No. 2920, Seq. ID No. 2922, Seq. ID No. 2924, Seq. ID No. 2926, Seq. ID No. 2928, Seq. ID No. 2930, Seq. ID No. 2932, Seq. ID No. 2934, Seq. ID No. 2944, Seq. ID No. 2946, Seq. ID No. 2954, Seq. ID No. 2956, Seq. ID No. 2958, Seq. ID No. 2960, Seq. ID No. 2962, Seq. ID No. 2964, Seq. ID No. 2966, Seq. ID No. 2968, Seq. ID No. 2972, Seq. ID No. 2980, Seq. ID No. 2986, Seq. ID No. 2990, Seq. ID No. 2994, Seq. ID No. 3000, Seq. ID No. 3002, Seq. ID No. 3004, Seq. ID No. 3016, Seq. ID No. 3018, Seq. ID No. 3020, Seq. ID No. 3022, Seq. ID No. 3034, Seq. ID No. 3036, Seq. ID No. 3038, Seq. ID No. 3040, Seq. ID No. 3042, Seq. ID No. 3046, Seq. ID No. 3048, Seq. ID No. 3050, Seq. ID No. 3052, Seq. ID No. 3054, Seq. ID No. 3058, Seq. ID No. 3060, Seq. ID No. 3062, Seq. ID No. 3066, Seq. ID No. 3068, Seq. ID No. 3070, Seq. ID No. 3080, Seq. ID No. 3082, Seq. ID No. 3084, Seq. ID No. 3086, Seq. ID No. 3088, Seq. ID No. 3090, Seq. ID No. 3092, Seq. ID No. 3094, Seq. ID No. 3096, Seq. ID No. 3098, Seq. ID No. 3100, Seq. ID No. 3102, Seq. ID No. 3104, Seq. ID No. 3106, Seq. ID No. 3108, Seq. ID No. 3110, Seq. ID No. 3112, Seq. ID No. 3114, Seq. ID No. 3116, Seq. ID No. 3118, Seq. ID No. 3126, Seq. ID No. 3128, Seq. ID No. 3130, Seq. ID No. 3132, Seq. ID No. 3134, Seq. ID No. 3136, Seq. ID No. 3138, Seq. ID No. 3140, Seq. ID No. 3142, Seq. ID No. 3144, Seq. ID No. 3148, Seq. ID No. 3150, Seq. ID No. 3152, Seq. ID No. 3154, Seq. ID No. 3160, Seq. ID No. 3162, Seq. ID No. 3168, Seq. ID No. 3170, Seq. ID No. 3172, Seq. ID No. 3176, Seq. ID No. 3178, Seq. ID No. 3180, Seq. ID No. 3182, Seq. ID No. 3186, Seq. ID No. 3188, Seq. ID No. 3190, Seq. ID No. 3192, Seq. ID No. 3196, Seq. ID No. 3198, Seq. ID No. 3200, Seq. ID No. 3204, Seq. ID No. 3206, Seq. ID No. 3208, Seq. ID No. 3210, Seq. ID No. 3212, Seq. ID No. 3222, Seq. ID No. 3224, Seq. ID No. 3226, Seq. ID No. 3228, Seq. ID No. 3230, Seq. ID No. 3232, Seq. ID No. 3234, Seq. ID No. 3236, Seq. ID No. 3238, Seq. ID No. 3240, Seq. ID No. 3248, Seq. ID No. 3252, Seq. ID No. 3254, Seq. ID No. 3260, Seq. ID No. 3266, Seq. ID No. 3268, Seq. ID No. 3272, Seq. ID No. 3274, Seq. ID No. 3280, Seq. ID No. 3282, Seq. ID No. 3284, Seq. ID No. 3286, Seq. ID No. 3288, Seq. ID No. 3290, Seq. ID No. 3292, Seq. ID No. 3294, Seq. ID No. 3296, Seq. ID No. 3300, Seq. ID No. 3302, Seq. ID No. 3304, Seq. ID No. 3306, Seq. ID No. 3308, Seq. ID No. 3310, Seq. ID No. 3312, Seq. ID No. 3314, Seq. ID No. 3316, Seq. ID No. 3320, Seq. ID No. 3322, Seq. ID No. 3328, Seq. ID No. 3330, Seq. ID No. 3334, Seq. ID No. 3342, Seq. ID No. 3344, Seq. ID No. 3346, Seq. ID No. 3348, Seq. ID No. 3362, Seq. ID No. 3364, Seq. ID No. 3366, Seq. ID No. 3368, Seq. ID No. 3370, Seq. ID No. 3372, Seq. ID No. 3374, Seq. ID No. 3380, Seq. ID No. 3382, Seq. ID No. 3384, Seq. ID No. 3386, Seq. ID No. 3388, Seq. ID No. 3394, Seq. ID No. 3398, Seq. ID No. 3400, Seq. ID No. 3402, Seq. ID No. 3404, Seq. ID No. 3408, Seq. ID No. 3410, Seq. ID No. 3412, Seq. ID No. 3414, Seq. ID No. 3418, Seq. ID No. 3420, Seq. ID No. 3424, Seq. ID No. 3430, Seq. ID No. 3432, Seq. ID No. 3436, Seq. ID No. 3438, Seq. ID No. 3440, Seq. ID No. 3444, Seq. ID No. 3446, Seq. ID No. 3448, Seq. ID No. 3460, Seq. ID No. 3462, Seq. ID No. 3466, Seq. ID No. 3468, Seq. ID No. 3470, Seq. ID No. 3472, Seq. ID No. 3474, Seq. ID No. 3476, Seq. ID No. 3478, Seq. ID No. 3480, Seq. ID No. 3484, Seq. ID No. 3486, Seq. ID No. 3488, Seq. ID No. 3490, Seq. ID No. 3496, Seq. ID No. 3498, Seq. ID No. 3500, Seq. ID No. 3502, Seq. ID No. 3508, Seq. ID No. 3510, Seq. ID No. 3512, Seq. ID No. 3514, Seq. ID No. 3516, Seq. ID No. 3522, Seq. ID No. 3524, Seq. ID No. 3526, Seq. ID No. 3530, Seq. ID No. 3534, Seq. ID No. 3536, Seq. ID No. 3538, Seq. ID No. 3542, Seq. ID No. 3544, Seq. ID No. 3546, Seq. ID No. 3548, Seq. ID No. 3550, Seq. ID No. 3552, Seq. ID No. 3554, Seq. ID No. 3556, Seq. ID No. 3558, Seq. ID No. 3560, Seq. ID No. 3564, Seq. ID No. 3566, Seq. ID No. 3568, Seq. ID No. 3570, Seq. ID No. 3572, Seq. ID No. 3576, Seq. ID No. 3578, Seq. ID No. 3580, Seq. ID No. 3584, Seq. ID No. 3586, Seq. ID No. 3590, Seq. ID No. 3592, Seq. ID No. 3598, Seq. ID No. 3600, Seq. ID No. 3602, Seq. ID No. 3604, Seq. ID No. 3606, Seq. ID No. 3608, Seq. ID No. 3610, Seq. ID No. 3614, Seq. ID No. 3616, Seq. ID No. 3624, Seq. ID No. 3628, Seq. ID No. 3632, Seq. ID No. 3634, Seq. ID No. 3638, Seq. ID No. 3640, Seq. ID No. 3642, Seq. ID No. 3644, Seq. ID No. 3646, Seq. ID No. 3648, Seq. ID No. 3650, Seq. ID No. 3652, Seq. ID No. 3654, Seq. ID No. 3656, Seq. ID No. 3658, Seq. ID No. 3660, Seq. ID No. 3662, Seq. ID No. 3664, Seq. ID No. 3666, Seq. ID No. 3668, Seq. ID No. 3670, Seq. ID No. 3672, Seq. ID No. 3674, Seq. ID No. 3676, Seq. ID No. 3682, Seq. ID No. 3684, Seq. ID No. 3686, Seq. ID No. 3688, Seq. ID No. 3692, Seq. ID No. 3694, Seq. ID No. 3696, Seq. ID No. 3698, Seq. ID No. 3700, Seq. ID No. 3702, Seq. ID No. 3704, Seq. ID No. 3706, Seq. ID No. 3708, Seq. ID No. 3710, Seq. ID No. 3712, Seq. ID No. 3714, Seq. ID No. 3716, Seq. ID No. 3720, Seq. ID No. 3722, Seq. ID No. 3726, Seq. ID No. 3730, Seq. ID No. 3732, Seq. ID No. 3734, Seq. ID No. 3736, Seq. ID No. 3738, Seq. ID No. 3740, Seq. ID No. 3744, Seq. ID No. 3748, Seq. ID No. 3750, Seq. ID No. 3752, Seq. ID No. 3760, Seq. ID No. 3768, Seq. ID No. 3770, Seq. ID No. 3772, Seq. ID No. 3774, Seq. ID No. 3776, Seq. ID No. 3778, Seq. ID No. 3780, Seq. ID No. 3786, Seq. ID No. 3788, Seq. ID No. 3790, Seq. ID No. 3792, Seq. ID No. 3796, Seq. ID No. 3798, Seq. ID No. 3802, Seq. ID No. 3804, Seq. ID No. 3806, Seq. ID No. 3810, Seq. ID No. 3812, Seq. ID No. 3820, Seq. ID No. 3822, Seq. ID No. 3824, Seq. ID No. 3826, Seq. ID No. 3828, Seq. ID No. 3830, Seq. ID No. 3832, Seq. ID No. 3834, Seq. ID No. 3836, Seq. ID No. 3842, Seq. ID No. 3844, Seq. ID No. 3846, Seq. ID No. 3848, Seq. ID No. 3856, Seq. ID No. 3858, Seq. ID No. 3868, Seq. ID No. 3870, Seq. ID No. 3872, Seq. ID No. 3874, Seq. ID No. 3876, Seq. ID No. 3878, Seq. ID No. 3882, Seq. ID No. 3884, Seq. ID No. 3886, Seq. ID No. 3888, Seq. ID No. 3894, Seq. ID No. 3898, Seq. ID No. 3900, Seq. ID No. 3902, Seq. ID No. 3906, Seq. ID No. 3908, Seq. ID No. 3910, Seq. ID No. 3912, Seq. ID No. 3914, Seq. ID No. 3918, Seq. ID No. 3920, Seq. ID No. 3922, Seq. ID No. 3928, Seq. ID No. 3930, Seq. ID No. 3932, Seq. ID No. 3934, Seq. ID No. 3936, Seq. ID No. 3938, Seq. ID No. 3940, Seq. ID No. 3942, Seq. ID No. 3944, Seq. ID No. 3946, Seq. ID No. 3948, Seq. ID No. 3950, Seq. ID No. 3952, Seq. ID No. 3954, Seq. ID No. 3956, Seq. ID No. 3958, Seq. ID No. 3962, Seq. ID No. 3966, Seq. ID No. 3968, Seq. ID No. 3970, Seq. ID No. 3972, Seq. ID No. 3978, Seq. ID No. 3980, Seq. ID No. 3982, Seq. ID No. 3984, Seq. ID No. 3986, Seq. ID No. 3988, Seq. ID No. 3990, Seq. ID No. 3992, Seq. ID No. 4000, Seq. ID No. 4002, Seq. ID No. 4004, Seq. ID No. 4012, Seq. ID No. 4014, Seq. ID No. 4016, Seq. ID No. 4018, Seq. ID No. 4020, Seq. ID No. 4022, Seq. ID No. 4024, Seq. ID No. 4030, Seq. ID No. 4032, Seq. ID No. 4034, Seq. ID No. 4036, Seq. ID No. 4038, Seq. ID No. 4052, Seq. ID No. 4054, Seq. ID No. 4056, Seq. ID No. 4058, Seq. ID No. 4060, Seq. ID No. 4064, Seq. ID No. 4066, Seq. ID No. 4068, Seq. ID No. 4070, Seq. ID No. 4076, Seq. ID No. 4078, Seq. ID No. 4086, Seq. ID No. 4088, Seq. ID No. 4090, Seq. ID No. 4092, Seq. ID No. 4094, Seq. ID No. 4096, Seq. ID No. 4098, Seq. ID No. 4100, Seq. ID No. 4106, Seq. ID No. 4108, Seq. ID No. 4110, Seq. ID No. 4114, Seq. ID No. 4116, Seq. ID No. 4118, Seq. ID No. 4120, Seq. ID No. 4122, Seq. ID No. 4124, Seq. ID No. 4126, Seq. ID No. 4128, Seq. ID No. 4130, Seq. ID No. 4132, Seq. ID No. 4148, Seq. ID No. 4150, Seq. ID No. 4156, Seq. ID No. 4158, Seq. ID No. 4160, Seq. ID No. 4162, Seq. ID No. 4164, Seq. ID No. 4166, Seq. ID No. 4168, Seq. ID No. 4170, Seq. ID No. 4172, Seq. ID No. 4174, Seq. ID No. 4176, Seq. ID No. 4178, Seq. ID No. 4180, Seq. ID No. 4184, Seq. ID No. 4186, Seq. ID No. 4188, Seq. ID No. 4190, Seq. ID No. 4192, Seq. ID No. 4194, Seq. ID No. 4196, Seq. ID No. 4198, Seq. ID No. 4200, Seq. ID No. 4202, Seq. ID No. 4204, Seq. ID No. 4206, Seq. ID No. 4208, Seq. ID No. 4210, Seq. ID No. 4212, Seq. ID No. 4214, Seq. ID No. 4216, Seq. ID No. 4218, Seq. ID No. 4220, Seq. ID No. 4230, Seq. ID No. 4236, Seq. ID No. 4238, Seq. ID No. 4240, Seq. ID No. 4244, Seq. ID No. 4246, Seq. ID No. 4250, Seq. ID No. 4252, Seq. ID No. 4256, Seq. ID No. 4268, Seq. ID No. 4272, Seq. ID No. 4274, Seq. ID No. 4278, Seq. ID No. 4280, Seq. ID No. 4282, Seq. ID No. 4284, Seq. ID No. 4290, Seq. ID No. 4292, Seq. ID No. 4294, Seq. ID No. 4296, Seq. ID No. 4300, Seq. ID No. 4302, Seq. ID No. 4304, Seq. ID No. 4306, Seq. ID No. 4310, Seq. ID No. 4316, Seq. ID No. 4318, Seq. ID No. 4320, Seq. ID No. 4322, Seq. ID No. 4324, Seq. ID No. 4326, Seq. ID No. 4328, Seq. ID No. 4330, Seq. ID No. 4332, Seq. ID No. 4334, Seq. ID No. 4336, Seq. ID No. 4338, Seq. ID No. 4340, Seq. ID No. 4342, Seq. ID No. 4346, Seq. ID No. 4348, Seq. ID No. 4350, Seq. ID No. 4352, Seq. ID No. 4354, Seq. ID No. 4356, Seq. ID No. 4358, Seq. ID No. 4360, Seq. ID No. 4362, Seq. ID No. 4364, Seq. ID No. 4366, Seq. ID No. 4368, Seq. ID No. 4370, Seq. ID No. 4372, Seq. ID No. 4374, Seq. ID No. 4376, Seq. ID No. 4378, Seq. ID No. 4382, Seq. ID No. 4384, Seq. ID No. 4388, Seq. ID No. 4394, Seq. ID No. 4396, Seq. ID No. 4398, Seq. ID No. 4402, Seq. ID No. 4406, Seq. ID No. 4408, Seq. ID No. 4410, Seq. ID No. 4412, Seq. ID No. 4414, Seq. ID No. 4416, Seq. ID No. 4418, Seq. ID No. 4420, Seq. ID No. 4422, Seq. ID No. 4424, Seq. ID No. 4426, Seq. ID No. 4428, Seq. ID No. 4430, Seq. ID No. 4432, Seq. ID No. 4434, Seq. ID No. 4436, Seq. ID No. 4444, Seq. ID No. 4446, Seq. ID No. 4448, Seq. ID No. 4450, Seq. ID No. 4452, Seq. ID No. 4454, Seq. ID No. 4456, Seq. ID No. 4458, Seq. ID No. 4460, Seq. ID No. 4462, Seq. ID No. 4464, Seq. ID No. 4466, Seq. ID No. 4468, Seq. ID No. 4472, Seq. ID No. 4476, Seq. ID No. 4478, Seq. ID No. 4480, Seq. ID No. 4488, Seq. ID No. 4490, Seq. ID No. 4492, Seq. ID No. 4500, Seq. ID No. 4502, Seq. ID No. 4504, Seq. ID No. 4506, Seq. ID No. 4508, Seq. ID No. 4510, Seq. ID No. 4512, Seq. ID No. 4518, Seq. ID No. 4520, Seq. ID No. 4522, Seq. ID No. 4524, Seq. ID No. 4526, Seq. ID No. 4532, Seq. ID No. 4536, Seq. ID No. 4538, Seq. ID No. 4540, Seq. ID No. 4542, Seq. ID No. 4544, Seq. ID No. 4546, Seq. ID No. 4548, Seq. ID No. 4550, Seq. ID No. 4552, Seq. ID No. 4554, Seq. ID No. 4556, Seq. ID No. 4558, Seq. ID No. 4560, Seq. ID No. 4562, Seq. ID No. 4564, Seq. ID No. 4566, Seq. ID No. 4568, Seq. ID No. 4570, Seq. ID No. 4572, Seq. ID No. 4574, Seq. ID No. 4576, Seq. ID No. 4578, Seq. ID No. 4580, Seq. ID No. 4582, Seq. ID No. 4584, Seq. ID No. 4586, Seq. ID No. 4592, Seq. ID No. 4594, Seq. ID No. 4596, Seq. ID No. 4598, Seq. ID No. 4600, Seq. ID No. 4602, Seq. ID No. 4604, Seq. ID No. 4606, Seq. ID No. 4608, Seq. ID No. 4610, Seq. ID No. 4612, Seq. ID No. 4614, Seq. ID No. 4616, Seq. ID No. 4618, Seq. ID No. 4620, Seq. ID No. 4622, Seq. ID No. 4624, Seq. ID No. 4626, Seq. ID No. 4628, Seq. ID No. 4630, Seq. ID No. 4632, Seq. ID No. 4634, Seq. ID No. 4636, Seq. ID No. 4638, Seq. ID No. 4646, Seq. ID No. 4648, Seq. ID No. 4650, Seq. ID No. 4652, Seq. ID No. 4654, Seq. ID No. 4656, Seq. ID No. 4658, Seq. ID No. 4662, Seq. ID No. 4664, Seq. ID No. 4666, Seq. ID No. 4668, Seq. ID No. 4670, Seq. ID No. 4672, Seq. ID No. 4674, Seq. ID No. 4676, Seq. ID No. 4678, Seq. ID No. 4680, Seq. ID No. 4684, Seq. ID No. 4686, Seq. ID No. 4690, Seq. ID No. 4692, Seq. ID No. 4694, Seq. ID No. 4698, Seq. ID No. 4700, Seq. ID No. 4702, Seq. ID No. 4704, Seq. ID No. 4714, Seq. ID No. 4720, Seq. ID No. 4722, Seq. ID No. 4724, Seq. ID No. 4726, Seq. ID No. 4728, Seq. ID No. 4730, Seq. ID No. 4732, Seq. ID No. 4734, Seq. ID No. 4736, Seq. ID No. 4740, Seq. ID No. 4742, Seq. ID No. 4744, Seq. ID No. 4746, Seq. ID No. 4748, Seq. ID No. 4750, Seq. ID No. 4752, Seq. ID No. 4754, Seq. ID No. 4756, Seq. ID No. 4758, Seq. ID No. 4762, Seq. ID No. 4764, Seq. ID No. 4766, Seq. ID No. 4768, Seq. ID No. 4770, Seq. ID No. 4772, Seq. ID No. 4774, Seq. ID No. 4776, Seq. ID No. 4778, Seq. ID No. 4780, Seq. ID No. 4782, Seq. ID No. 4784, Seq. ID No. 4786, Seq. ID No. 4788, Seq. ID No. 4790, Seq. ID No. 4792, Seq. ID No. 4794, Seq. ID No. 4796, Seq. ID No. 4798, Seq. ID No. 4800, Seq. ID No. 4802, Seq. ID No. 4804, Seq. ID No. 4806, Seq. ID No. 4808, Seq. ID No. 4810, Seq. ID No. 4812, Seq. ID No. 4816, Seq. ID No. 4818, Seq. ID No. 4820, Seq. ID No. 4822, Seq. ID No. 4824, Seq. ID No. 4826, Seq. ID No. 4830, Seq. ID No. 4832, Seq. ID No. 4834, Seq. ID No. 4836, Seq. ID No. 4838, Seq. ID No. 4840, Seq. ID No. 4842, Seq. ID No. 4844, Seq. ID No. 4846, Seq. ID No. 4848, Seq. ID No. 4850, Seq. ID No. 4852, Seq. ID No. 4856, Seq. ID No. 4858, Seq. ID No. 4860, Seq. ID No. 4862, Seq. ID No. 4866, Seq. ID No. 4868, Seq. ID No. 4878, Seq. ID No. 4880, Seq. ID No. 4882, Seq. ID No. 4884, Seq. ID No. 4886, Seq. ID No. 4888, Seq. ID No. 4890, Seq. ID No. 4892, Seq. ID No. 4894, Seq. ID No. 4896, Seq. ID No. 4898, Seq. ID No. 4900, Seq. ID No. 4902, Seq. ID No. 4904, Seq. ID No. 4906, Seq. ID No. 4908, Seq. ID No. 4910, Seq. ID No. 4912, Seq. ID No. 4914, Seq. ID No. 4916, Seq. ID No. 4918, Seq. ID No. 4926, Seq. ID No. 4928, Seq. ID No. 4934, Seq. ID No. 4936, Seq. ID No. 4938, Seq. ID No. 4940, Seq. ID No. 4942, Seq. ID No. 4944, Seq. ID No. 4946, Seq. ID No. 4948, Seq. ID No. 4954, Seq. ID No. 4956, Seq. ID No. 4958, Seq. ID No. 4960, Seq. ID No. 4962, Seq. ID No. 4964, Seq. ID No. 4966, Seq. ID No. 4968, Seq. ID No. 4970, Seq. ID No. 4972, Seq. ID No. 4974, Seq. ID No. 4978, Seq. ID No. 4980, Seq. ID No. 4982, Seq. ID No. 4984, Seq. ID No. 4986, Seq. ID No. 4990, Seq. ID No. 4992, Seq. ID No. 4994, Seq. ID No. 4996, Seq. ID No. 4998, Seq. ID No. 5002, Seq. ID No. 5004, Seq. ID No. 5006, Seq. ID No. 5008, Seq. ID No. 5010, Seq. ID No. 5012, Seq. ID No. 5016, Seq. ID No. 5018, Seq. ID No. 5020, Seq. ID No. 5022, Seq. ID No. 5024, Seq. ID No. 5026, Seq. ID No. 5028, Seq. ID No. 5030, Seq. ID No. 5034, Seq. ID No. 5036, Seq. ID No. 5038, Seq. ID No. 5040, Seq. ID No. 5042, Seq. ID No. 5044, Seq. ID No. 5046, Seq. ID No. 5048, Seq. ID No. 5050, Seq. ID No. 5052, Seq. ID No. 5054, Seq. ID No. 5056, Seq. ID No. 5058, Seq. ID No. 5060, Seq. ID No. 5066, Seq. ID No. 5068, Seq. ID No. 5072, Seq. ID No. 5074, Seq. ID No. 5076, Seq. ID No. 5078, Seq. ID No. 5080, Seq. ID No. 5082, Seq. ID No. 5084, Seq. ID No. 5088, Seq. ID No. 5090, Seq. ID No. 5092, Seq. ID No. 5094, Seq. ID No. 5096, Seq. ID No. 5098, Seq. ID No. 5100, Seq. ID No. 5102, Seq. ID No. 5106, Seq. ID No. 5108, Seq. ID No. 5110, Seq. ID No. 5112, Seq. ID No. 5114, Seq. ID No. 5118, Seq. ID No. 5120, Seq. ID No. 5122, Seq. ID No. 5128, Seq. ID No. 5144, Seq. ID No. 5146, Seq. ID No. 5148, Seq. ID No. 5150, Seq. ID No. 5152, Seq. ID No. 5154, Seq. ID No. 5156, Seq. ID No. 5158, Seq. ID No. 5162, Seq. ID No. 5164, Seq. ID No. 5168, Seq. ID No. 5170, Seq. ID No. 5172, Seq. ID No. 5174, Seq. ID No. 5178, Seq. ID No. 5180, Seq. ID No. 5182, Seq. ID No. 5184, Seq. ID No. 5186, Seq. ID No. 5188, Seq. ID No. 5190, Seq. ID No. 5192, Seq. ID No. 5194, Seq. ID No. 5196, Seq. ID No. 5202, Seq. ID No. 5204, Seq. ID No. 5210, Seq. ID No. 5212, Seq. ID No. 5216, Seq. ID No. 5218, Seq. ID No. 5222, Seq. ID No. 5226, Seq. ID No. 5228, Seq. ID No. 5230, Seq. ID No. 5232, Seq. ID No. 5234, Seq. ID No. 5236, Seq. ID No. 5238, Seq. ID No. 5240, Seq. ID No. 5242, Seq. ID No. 5244, Seq. ID No. 5246, Seq. ID No. 5248, Seq. ID No. 5250, Seq. ID No. 5252, Seq. ID No. 5254, Seq. ID No. 5256, Seq. ID No. 5258, Seq. ID No. 5260, Seq. ID No. 5262, Seq. ID No. 5264, Seq. ID No. 5266, Seq. ID No. 5270, Seq. ID No. 5272, Seq. ID No. 5284, Seq. ID No. 5288, Seq. ID No. 5290, Seq. ID No. 5292, Seq. ID No. 5294, Seq. ID No. 5296, Seq. ID No. 5298, Seq. ID No. 5300, Seq. ID No. 5302, Seq. ID No. 5304, Seq. ID No. 5306, Seq. ID No. 5308, Seq. ID No. 5310, Seq. ID No. 5312, Seq. ID No. 5314, Seq. ID No. 5316, Seq. ID No. 5318, Seq. ID No. 5320, Seq. ID No. 5322, Seq. ID No. 5324, Seq. ID No. 5326, Seq. ID No. 5334, Seq. ID No. 5336, Seq. ID No. 5338, Seq. ID No. 5340, Seq. ID No. 5344, Seq. ID No. 5346, Seq. ID No. 5348, Seq. ID No. 5350, Seq. ID No. 5352, Seq. ID No. 5354, Seq. ID No. 5356, Seq. ID No. 5358, Seq. ID No. 5360, Seq. ID No. 5362, Seq. ID No. 5364, Seq. ID No. 5366, Seq. ID No. 5368, Seq. ID No. 5370, Seq. ID No. 5372, Seq. ID No. 5374, Seq. ID No. 5376, Seq. ID No. 5382, Seq. ID No. 5384, Seq. ID No. 5390, Seq. ID No. 5392, Seq. ID No. 5394, Seq. ID No. 5396, Seq. ID No. 5398, Seq. ID No. 5400, Seq. ID No. 5402, Seq. ID No. 5404, Seq. ID No. 5408, Seq. ID No. 5410, Seq. ID No. 5412, Seq. ID No. 5418, Seq. ID No. 5420, Seq. ID No. 5422, Seq. ID No. 5424, Seq. ID No. 5426, Seq. ID No. 5428, Seq. ID No. 5430, Seq. ID No. 5432, Seq. ID No. 5436, Seq. ID No. 5438, Seq. ID No. 5440, Seq. ID No. 5442, Seq. ID No. 5444, Seq. ID No. 5446, Seq. ID No. 5448, Seq. ID No. 5450, Seq. ID No. 5452, Seq. ID No. 5456, Seq. ID No. 5458, Seq. ID No. 5468, Seq. ID No. 5470, Seq. ID No. 5472, Seq. ID No. 5474, Seq. ID No. 5476, Seq. ID No. 5478, Seq. ID No. 5480, Seq. ID No. 5482, Seq. ID No. 5484, Seq. ID No. 5486, Seq. ID No. 5488, Seq. ID No. 5490, Seq. ID No. 5494, Seq. ID No. 5496, Seq. ID No. 5500, Seq. ID No. 5502, Seq. ID No. 5504, Seq. ID No. 5506, Seq. ID No. 5508, Seq. ID No. 5512, Seq. ID No. 5520, Seq. ID No. 5522, Seq. ID No. 5524, Seq. ID No. 5528, Seq. ID No. 5536, Seq. ID No. 5538, Seq. ID No. 5540, Seq. ID No. 5542, Seq. ID No. 5548, Seq. ID No. 5550, Seq. ID No. 5552, Seq. ID No. 5554, Seq. ID No. 5556, Seq. ID No. 5558, Seq. ID No. 5560, Seq. ID No. 5562, Seq. ID No. 5564, Seq. ID No. 5566, Seq. ID No. 5568, Seq. ID No. 5572, Seq. ID No. 5574, Seq. ID No. 5576, Seq. ID No. 5578, Seq. ID No. 5580, Seq. ID No. 5582, Seq. ID No. 5584, Seq. ID No. 5586, Seq. ID No. 5600, Seq. ID No. 5602, Seq. ID No. 5604, Seq. ID No. 5606, Seq. ID No. 5608, Seq. ID No. 5614, Seq. ID No. 5616, Seq. ID No. 5618, Seq. ID No. 5620, Seq. ID No. 5628, Seq. ID No. 5630, Seq. ID No. 5632, Seq. ID No. 5636, Seq. ID No. 5646, Seq. ID No. 5648, Seq. ID No. 5652, Seq. ID No. 5654, Seq. ID No. 5656, Seq. ID No. 5658, Seq. ID No. 5662, Seq. ID No. 5664, Seq. ID No. 5666, Seq. ID No. 5670, Seq. ID No. 5672, Seq. ID No. 5674, Seq. ID No. 5676, Seq. ID No. 5678, Seq. ID No. 5680, Seq. ID No. 5692, Seq. ID No. 5694, Seq. ID No. 5696, Seq. ID No. 5698, Seq. ID No. 5702, Seq. ID No. 5708, Seq. ID No. 5714, Seq. ID No. 5716, Seq. ID No. 5718, Seq. ID No. 5722, Seq. ID No. 5724, Seq. ID No. 5726, Seq. ID No. 5728, Seq. ID No. 5732, Seq. ID No. 5738, Seq. ID No. 5742, Seq. ID No. 5744, Seq. ID No. 5746, Seq. ID No. 5748, Seq. ID No. 5750, Seq. ID No. 5752, Seq. ID No. 5754, Seq. ID No. 5756, Seq. ID No. 5758, Seq. ID No. 5760, Seq. ID No. 5762, Seq. ID No. 5770, Seq. ID No. 5774, Seq. ID No. 5776, Seq. ID No. 5778, Seq. ID No. 5780, Seq. ID No. 5782, Seq. ID No. 5788, Seq. ID No. 5790, Seq. ID No. 5794, Seq. ID No. 5796, Seq. ID No. 5800, Seq. ID No. 5802, Seq. ID No. 5804, Seq. ID No. 5806, Seq. ID No. 5808, Seq. ID No. 5810, Seq. ID No. 5812, Seq. ID No. 5814, Seq. ID No. 5816, Seq. ID No. 5818, Seq. ID No. 5820, Seq. ID No. 5822, Seq. ID No. 5824, Seq. ID No. 5826, Seq. ID No. 5828, Seq. ID No. 5832, Seq. ID No. 5834, Seq. ID No. 5836, Seq. ID No. 5838, Seq. ID No. 5840, Seq. ID No. 5842, Seq. ID No. 5844, Seq. ID No. 5846, Seq. ID No. 5848, Seq. ID No. 5850, Seq. ID No. 5852, Seq. ID No. 5854, Seq. ID No. 5856, Seq. ID No. 5858, Seq. ID No. 5860, Seq. ID No. 5862, Seq. ID No. 5864, Seq. ID No. 5866, Seq. ID No. 5868, Seq. ID No. 5870, Seq. ID No. 5872, Seq. ID No. 5874, Seq. ID No. 5876, Seq. ID No. 5878, Seq. ID No. 5882, Seq. ID No. 5888, Seq. ID No. 5892, Seq. ID No. 5898, Seq. ID No. 5904, Seq. ID No. 5908, Seq. ID No. 5912, Seq. ID No. 5914, Seq. ID No. 5916, Seq. ID No. 5918, Seq. ID No. 5920, Seq. ID No. 5926, Seq. ID No. 5932, Seq. ID No. 5934, Seq. ID No. 5936, Seq. ID No. 5938, Seq. ID No. 5942, Seq. ID No. 5944, Seq. ID No. 5946, Seq. ID No. 5948, Seq. ID No. 5950, Seq. ID No. 5952, Seq. ID No. 5954, Seq. ID No. 5956, Seq. ID No. 5958, Seq. ID No. 5960, Seq. ID No. 5962, Seq. ID No. 5964, Seq. ID No. 5966, Seq. ID No. 5968, Seq. ID No. 5970, Seq. ID No. 5972, Seq. ID No. 5974, Seq. ID No. 5976, Seq. ID No. 5978, Seq. ID No. 5980, Seq. ID No. 5982, Seq. ID No. 5984, Seq. ID No. 5986, Seq. ID No. 5988, Seq. ID No. 5990, Seq. ID No. 5992, Seq. ID No. 5994, Seq. ID No. 5996, Seq. ID No. 5998, Seq. ID No. 6000, Seq. ID No. 6002, Seq. ID No. 6004, Seq. ID No. 6006, Seq. ID No. 6008, Seq. ID No. 6010, Seq. ID No. 6012, Seq. ID No. 6014, Seq. ID No. 6016, Seq. ID No. 6018, Seq. ID No. 6020, Seq. ID No. 6022, Seq. ID No. 6030, Seq. ID No. 6032, Seq. ID No. 6034, Seq. ID No. 6036, Seq. ID No. 6038, Seq. ID No. 6040, Seq. ID No. 6042, Seq. ID No. 6046, Seq. ID No. 6050, Seq. ID No. 6052, Seq. ID No. 6056, Seq. ID No. 6058, Seq. ID No. 6062, Seq. ID No. 6066, Seq. ID No. 6070, Seq. ID No. 6072, Seq. ID No. 6074, Seq. ID No. 6076, Seq. ID No. 6078, Seq. ID No. 6080, Seq. ID No. 6082, Seq. ID No. 6084, Seq. ID No. 6086, Seq. ID No. 6094, Seq. ID No. 6098, Seq. ID No. 6100, Seq. ID No. 6102, Seq. ID No. 6106, Seq. ID No. 6114, Seq. ID No. 6120, Seq. ID No. 6122, Seq. ID No. 6124, Seq. ID No. 6126, Seq. ID No. 6128, Seq. ID No. 6130, Seq. ID No. 6132, Seq. ID No. 6134, Seq. ID No. 6140, Seq. ID No. 6142, Seq. ID No. 6144, Seq. ID No. 6146, Seq. ID No. 6148, Seq. ID No. 6150, Seq. ID No. 6152, Seq. ID No. 6154, Seq. ID No. 6156, Seq. ID No. 6158, Seq. ID No. 6168, Seq. ID No. 6172, Seq. ID No. 6174, Seq. ID No. 6176, Seq. ID No. 6178, Seq. ID No. 6180, Seq. ID No. 6182, Seq. ID No. 6194, Seq. ID No. 6196, Seq. ID No. 6200, Seq. ID No. 6202, Seq. ID No. 6208, Seq. ID No. 6212, Seq. ID No. 6214, Seq. ID No. 6216, Seq. ID No. 6220, Seq. ID No. 6232, Seq. ID No. 6234, Seq. ID No. 6240, Seq. ID No. 6248, Seq. ID No. 6252, Seq. ID No. 6256, Seq. ID No. 6258, Seq. ID No. 6262, Seq. ID No. 6270, Seq. ID No. 6272, Seq. ID No. 6278, Seq. ID No. 6280, Seq. ID No. 6282, Seq. ID No. 6288, Seq. ID No. 6292, Seq. ID No. 6294, Seq. ID No. 6296, Seq. ID No. 6298, Seq. ID No. 6300, Seq. ID No. 6306, Seq. ID No. 6308, Seq. ID No. 6310, Seq. ID No. 6312, Seq. ID No. 6314, Seq. ID No. 6316, Seq. ID No. 6318, Seq. ID No. 6320, Seq. ID No. 6322, Seq. ID No. 6324, Seq. ID No. 6330, Seq. ID No. 6332, Seq. ID No. 6334, Seq. ID No. 6336, Seq. ID No. 6338, Seq. ID No. 6340, Seq. ID No. 6342, Seq. ID No. 6344, Seq. ID No. 6346, Seq. ID No. 6348, Seq. ID No. 6350, Seq. ID No. 6352, Seq. ID No. 6354, Seq. ID No. 6356, Seq. ID No. 6358, Seq. ID No. 6360, Seq. ID No. 6362, Seq. ID No. 6368, Seq. ID No. 6370, Seq. ID No. 6372, Seq. ID No. 6374, Seq. ID No. 6376, Seq. ID No. 6380, Seq. ID No. 6386, Seq. ID No. 6388, Seq. ID No. 6390, Seq. ID No. 6392, Seq. ID No. 6394, Seq. ID No. 6396, Seq. ID No. 6398, Seq. ID No. 6400, Seq. ID No. 6402, Seq. ID No. 6404, Seq. ID No. 6406, Seq. ID No. 6408, Seq. ID No. 6410, Seq. ID No. 6412, Seq. ID No. 6414, Seq. ID No. 6418, Seq. ID No. 6420, Seq. ID No. 6422, Seq. ID No. 6424, Seq. ID No. 6434, Seq. ID No. 6436, Seq. ID No. 6438, Seq. ID No. 6440, Seq. ID No. 6442, Seq. ID No. 6444, Seq. ID No. 6446, Seq. ID No. 6448, Seq. ID No. 6450, Seq. ID No. 6452, Seq. ID No. 6454, Seq. ID No. 6456, Seq. ID No. 6458, Seq. ID No. 6460, Seq. ID No. 6462, Seq. ID No. 6464, Seq. ID No. 6466, Seq. ID No. 6468, Seq. ID No. 6470, Seq. ID No. 6472, Seq. ID No. 6476, Seq. ID No. 6478, Seq. ID No. 6482, Seq. ID No. 6484, Seq. ID No. 6486, Seq. ID No. 6488, Seq. ID No. 6490, Seq. ID No. 6492, Seq. ID No. 6494, Seq. ID No. 6496, Seq. ID No. 6500, Seq. ID No. 6502, Seq. ID No. 6504, Seq. ID No. 6506, Seq. ID No. 6508, Seq. ID No. 6510, Seq. ID No. 6512, Seq. ID No. 6514, Seq. ID No. 6516, Seq. ID No. 6520, Seq. ID No. 6522, Seq. ID No. 6524, Seq. ID No. 6526, Seq. ID No. 6528, Seq. ID No. 6530, Seq. ID No. 6532, Seq. ID No. 6534, Seq. ID No. 6536, Seq. ID No. 6538, Seq. ID No. 6540, Seq. ID No. 6546, Seq. ID No. 6548, Seq. ID No. 6550, Seq. ID No. 6552, Seq. ID No. 6554, Seq. ID No. 6560, Seq. ID No. 6562, Seq. ID No. 6564, Seq. ID No. 6566, Seq. ID No. 6568, Seq. ID No. 6570, Seq. ID No. 6576, Seq. ID No. 6578, Seq. ID No. 6580, Seq. ID No. 6584, Seq. ID No. 6586, Seq. ID No. 6588, Seq. ID No. 6590, Seq. ID No. 6592, Seq. ID No. 6606, Seq. ID No. 6608, Seq. ID No. 6610, Seq. ID No. 6612, Seq. ID No. 6614, Seq. ID No. 6616, Seq. ID No. 6618, Seq. ID No. 6620, Seq. ID No. 6622, Seq. ID No. 6624, Seq. ID No. 6626, Seq. ID No. 6628, Seq. ID No. 6630, Seq. ID No. 6634, Seq. ID No. 6636, Seq. ID No. 6642, and Seq. ID No. 6648.

41. The Alloiococcus otitidis polypeptide of claim 36, wherein the polypeptide is identified by BlastP analysis, and is selected from one of Seq. ID No. 10, Seq. ID No. 18, Seq. ID No. 46, Seq. ID No. 50, Seq. ID No. 54, Seq. ID No. 60, Seq. ID No. 90, Seq. ID No. 146, Seq. ID No. 158, Seq. ID No. 168, Seq. ID No. 172, Seq. ID No. 192, Seq. ID No. 216, Seq. ID No. 218, Seq. ID No. 226, Seq. ID No. 240, Seq. ID No. 254, Seq. ID No. 274, Seq. ID No. 278, Seq. ID No. 290, Seq. ID No. 330, Seq. ID No. 344, Seq. ID No. 346, Seq. ID No. 376, Seq. ID No. 502, Seq. ID No. 506, Seq. ID No. 512, Seq. ID No. 516, Seq. ID No. 530, Seq. ID No. 570, Seq. ID No. 576, Seq. ID No. 594, Seq. ID No. 596, Seq. ID No. 638, Seq. ID No. 660, Seq. ID No. 670, Seq. ID No. 676, Seq. ID No. 682, Seq. ID No. 730, Seq. ID No. 780, Seq. ID No. 820, Seq. ID No. 896, Seq. ID No. 922, Seq. ID No. 936, Seq. ID No. 956, Seq. ID No. 968, Seq. ID No. 986, Seq. ID No. 1018, Seq. ID No. 1040, Seq. ID No. 1076, Seq. ID No. 1090, Seq. ID No. 1138, Seq. ID No. 1224, Seq. ID No. 1242, Seq. ID No. 1316, Seq. ID No. 1332, Seq. ID No. 1370, Seq. ID No. 1460, Seq. ID No. 1518, Seq. ID No. 1562, Seq. ID No. 1564, Seq. ID No. 1568, Seq. ID No. 1572, Seq. ID No. 1574, Seq. ID No. 1602, Seq. ID No. 1608, Seq. ID No. 1614, Seq. ID No. 1620, Seq. ID No. 1642, Seq. ID No. 1648, Seq. ID No. 1654, Seq. ID No. 1658, Seq. ID No. 1666, Seq. ID No. 1700, Seq. ID No. 1714, Seq. ID No. 1752, Seq. ID No. 1772, Seq. ID No. 1786, Seq. ID No. 1790, Seq. ID No. 1792, Seq. ID No. 1798, Seq. ID No. 1812, Seq. ID No. 1818, Seq. ID No. 1820, Seq. ID No. 1822, Seq. ID No. 1872, Seq. ID No. 1896, Seq. ID No. 1906, Seq. ID No. 1914, Seq. ID No. 1920, Seq. ID No. 1924, Seq. ID No. 1966, Seq. ID No. 1972, Seq. ID No. 2000, Seq. ID No. 2048, Seq. ID No. 2056, Seq. ID No. 2092, Seq. ID No. 2094, Seq. ID No. 2116, Seq. ID No. 2120, Seq. ID No. 2128, Seq. ID No. 2166, Seq. ID No. 2180, Seq. ID No. 2208, Seq. ID No. 2224, Seq. ID No. 2228, Seq. ID No. 2242, Seq. ID No. 2254, Seq. ID No. 2264, Seq. ID No. 2270, Seq. ID No. 2276, Seq. ID No. 2302, Seq. ID No. 2316, Seq. ID No. 2330, Seq. ID No. 2334, Seq. ID No. 2344, Seq. ID No. 2346, Seq. ID No. 2364, Seq. ID No. 2374, Seq. ID No. 2392, Seq. ID No. 2418, Seq. ID No. 2430, Seq. ID No. 2466, Seq. ID No. 2476, Seq. ID No. 2484, Seq. ID No. 2514, Seq. ID No. 2526, Seq. ID No. 2590, Seq. ID No. 2614, Seq. ID No. 2652, Seq. ID No. 2670, Seq. ID No. 2678, Seq. ID No. 2688, Seq. ID No. 2690, Seq. ID No. 2694, Seq. ID No. 2704, Seq. ID No. 2710, Seq. ID No. 2716, Seq. ID No. 2752, Seq. ID No. 2754, Seq. ID No. 2768, Seq. ID No. 2788, Seq. ID No. 2792, Seq. ID No. 2804, Seq. ID No. 2868, Seq. ID No. 2872, Seq. ID No. 2908, Seq. ID No. 2920, Seq. ID No. 2922, Seq. ID No. 2924, Seq. ID No. 2928, Seq. ID No. 2934, Seq. ID No. 2958, Seq. ID No. 2980, Seq. ID No. 3000, Seq. ID No. 3006, Seq. ID No. 3020, Seq. ID No. 3032, Seq. ID No. 3036, Seq. ID No. 3056, Seq. ID No. 3068, Seq. ID No. 3086, Seq. ID No. 3166, Seq. ID No. 3184, Seq. ID No. 3190, Seq. ID No. 3198, Seq. ID No. 3212, Seq. ID No. 3252, Seq. ID No. 3258, Seq. ID No. 3260, Seq. ID No. 3298, Seq. ID No. 3320, Seq. ID No. 3324, Seq. ID No. 3336, Seq. ID No. 3338, Seq. ID No. 3356, Seq. ID No. 3360, Seq. ID No. 3378, Seq. ID No. 3398, Seq. ID No. 3406, Seq. ID No. 3416, Seq. ID No. 3426, Seq. ID No. 3428, Seq. ID No. 3462, Seq. ID No. 3464, Seq. ID No. 3488, Seq. ID No. 3494, Seq. ID No. 3498, Seq. ID No. 3508, Seq. ID No. 3520, Seq. ID No. 3522, Seq. ID No. 3530, Seq. ID No. 3536, Seq. ID No. 3630, Seq. ID No. 3636, Seq. ID No. 3728, Seq. ID No. 3760, Seq. ID No. 3772, Seq. ID No. 3794, Seq. ID No. 3840, Seq. ID No. 3852, Seq. ID No. 3864, Seq. ID No. 3866, Seq. ID No. 3884, Seq. ID No. 3892, Seq. ID No. 3914, Seq. ID No. 3926, Seq. ID No. 3930, Seq. ID No. 3976, Seq. ID No. 3982, Seq. ID No. 4028, Seq. ID No. 4040, Seq. ID No. 4062, Seq. ID No. 4074, Seq. ID No. 4082, Seq. ID No. 4104, Seq. ID No. 4112, Seq. ID No. 4138, Seq. ID No. 4226, Seq. ID No. 4248, Seq. ID No. 4344, Seq. ID No. 4380, Seq. ID No. 4390, Seq. ID No. 4470, Seq. ID No. 4474, Seq. ID No. 4482, Seq. ID No. 4486, Seq. ID No. 4494, Seq. ID No. 4530, Seq. ID No. 4644, Seq. ID No. 4706, Seq. ID No. 4714, Seq. ID No. 4718, Seq. ID No. 4814, Seq. ID No. 4816, Seq. ID No. 4826, Seq. ID No. 4828, Seq. ID No. 4834, Seq. ID No. 4838, Seq. ID No. 4852, Seq. ID No. 4864, Seq. ID No. 4876, Seq. ID No. 4880, Seq. ID No. 4894, Seq. ID No. 4902, Seq. ID No. 4920, Seq. ID No. 4932, Seq. ID No. 4960, Seq. ID No. 4964, Seq. ID No. 4986, Seq. ID No. 5114, Seq. ID No. 5116, Seq. ID No. 5118, Seq. ID No. 5126, Seq. ID No. 5142, Seq. ID No. 5184, Seq. ID No. 5202, Seq. ID No. 5206, Seq. ID No. 5214, Seq. ID No. 5220, Seq. ID No. 5280, Seq. ID No. 5286, Seq. ID No. 5332, Seq. ID No. 5382, Seq. ID No. 5390, Seq. ID No. 5406, Seq. ID No. 5412, Seq. ID No. 5420, Seq. ID No. 5434, Seq. ID No. 5464, Seq. ID No. 5478, Seq. ID No. 5492, Seq. ID No. 5510, Seq. ID No. 5514, Seq. ID No. 5526, Seq. ID No. 5530, Seq. ID No. 5538, Seq. ID No. 5568, Seq. ID No. 5600, Seq. ID No. 5622, Seq. ID No. 5638, Seq. ID No. 5700, Seq. ID No. 5724, Seq. ID No. 5734, Seq. ID No. 5762, Seq. ID No. 5792, Seq. ID No. 5896, Seq. ID No. 5902, Seq. ID No. 5904, Seq. ID No. 5910, Seq. ID No. 5914, Seq. ID No. 5920, Seq. ID No. 5930, Seq. ID No. 5940, Seq. ID No. 6002, Seq. ID No. 6044, Seq. ID No. 6054, Seq. ID No. 6058, Seq. ID No. 6090, Seq. ID No. 6096, Seq. ID No. 6104, Seq. ID No. 6112, Seq. ID No. 6118, Seq. ID No. 6166, Seq. ID No. 6170, Seq. ID No. 6186, Seq. ID No. 6192, Seq. ID No. 6210, Seq. ID No. 6230, Seq. ID No. 6258, Seq. ID No. 6264, Seq. ID No. 6272, Seq. ID No. 6276, Seq. ID No. 6290, Seq. ID No. 6304, Seq. ID No. 6366, Seq. ID No. 6370, Seq. ID No. 6384, and Seq. ID No. 6416.

42. The Alloiococcus otitidis polypeptide of claim 36, wherein the polypeptide is identified by Pfam analysis, and is selected from one of Seq. ID No. 90, Seq. ID No. 146, Seq. ID No. 156, Seq. ID No. 168, Seq. ID No. 174, Seq. ID No. 192, Seq. ID No. 216, Seq. ID No. 220, Seq. ID No. 222, Seq. ID No. 224, Seq. ID No. 226, Seq. ID No. 234, Seq. ID No. 240, Seq. ID No. 252, Seq. ID No. 254, Seq. ID No. 272, Seq. ID No. 292, Seq. ID No. 332, Seq. ID No. 342, Seq. ID No. 346, Seq. ID No. 376, Seq. ID No. 502, Seq. ID No. 506, Seq. ID No. 512, Seq. ID No. 530, Seq. ID No. 568, Seq. ID No. 570, Seq. ID No. 576, Seq. ID No. 594, Seq. ID No. 596, Seq. ID No. 602, Seq. ID No. 638, Seq. ID No. 650, Seq. ID No. 662, Seq. ID No. 670, Seq. ID No. 676, Seq. ID No. 682, Seq. ID No. 684, Seq. ID No. 730, Seq. ID No. 754, Seq. ID No. 780, Seq. ID No. 828, Seq. ID No. 840, Seq. ID No. 896, Seq. ID No. 940, Seq. ID No. 970, Seq. ID No. 986, Seq. ID No. 1016, Seq. ID No. 1040, Seq. ID No. 1076, Seq. ID No. 1138, Seq. ID No. 1156, Seq. ID No. 1164, Seq. ID No. 1176, Seq. ID No. 1224, Seq. ID No. 1232, Seq. ID No. 1238, Seq. ID No. 1242, Seq. ID No. 1262, Seq. ID No. 1332, Seq. ID No. 1340, Seq. ID No. 1370, Seq. ID No. 1478, Seq. ID No. 1518, Seq. ID No. 1532, Seq. ID No. 1540, Seq. ID No. 1550, Seq. ID No. 1564, Seq. ID No. 1568, Seq. ID No. 1572, Seq. ID No. 1574, Seq. ID No. 1590, Seq. ID No. 1596, Seq. ID No. 1648, Seq. ID No. 1658, Seq. ID No. 1772, Seq. ID No. 1786, Seq. ID No. 1790, Seq. ID No. 1820, Seq. ID No. 1822, Seq. ID No. 1970, Seq. ID No. 2016, Seq. ID No. 2048, Seq. ID No. 2088, Seq. ID No. 2090, Seq. ID No. 2094, Seq. ID No. 2116, Seq. ID No. 2118, Seq. ID No. 2128, Seq. ID No. 2142, Seq. ID No. 2180, Seq. ID No. 2208, Seq. ID No. 2224, Seq. ID No. 2254, Seq. ID No. 2276, Seq. ID No. 2302, Seq. ID No. 2316, Seq. ID No. 2330, Seq. ID No. 2344, Seq. ID No. 2350, Seq. ID No. 2364, Seq. ID No. 2382, Seq. ID No. 2392, Seq. ID No. 2418, Seq. ID No. 2422, Seq. ID No. 2424, Seq. ID No. 2430, Seq. ID No. 2482, Seq. ID No. 2488, Seq. ID No. 2490, Seq. ID No. 2492, Seq. ID No. 2510, Seq. ID No. 2520, Seq. ID No. 2526, Seq. ID No. 2548, Seq. ID No. 2554, Seq. ID No. 2564, Seq. ID No. 2588, Seq. ID No. 2600, Seq. ID No. 2608, Seq. ID No. 2614, Seq. ID No. 2652, Seq. ID No. 2676, Seq. ID No. 2682, Seq. ID No. 2688, Seq. ID No. 2694, Seq. ID No. 2700, Seq. ID No. 2704, Seq. ID No. 2710, Seq. ID No. 2712, Seq. ID No. 2728, Seq. ID No. 2754, Seq. ID No. 2820, Seq. ID No. 2822, Seq. ID No. 2824, Seq. ID No. 2868, Seq. ID No. 2872, Seq. ID No. 2908, Seq. ID No. 2920, Seq. ID No. 2958, Seq. ID No. 2972, Seq. ID No. 2980, Seq. ID No. 3000, Seq. ID No. 3006, Seq. ID No. 3020, Seq. ID No. 3036, Seq. ID No. 3066, Seq. ID No. 3086, Seq. ID No. 3150, Seq. ID No. 3166, Seq. ID No. 3184, Seq. ID No. 3204, Seq. ID No. 3212, Seq. ID No. 3252, Seq. ID No. 3260, Seq. ID No. 3320, Seq. ID No. 3324, Seq. ID No. 3336, Seq. ID No. 3338, Seq. ID No. 3348, Seq. ID No. 3360, Seq. ID No. 3378, Seq. ID No. 3380, Seq. ID No. 3400, Seq. ID No. 3426, Seq. ID No. 3484, Seq. ID No. 3496, Seq. ID No. 3498, Seq. ID No. 3520, Seq. ID No. 3530, Seq. ID No. 3600, Seq. ID No. 3612, Seq. ID No. 3616, Seq. ID No. 3620, Seq. ID No. 3664, Seq. ID No. 3740, Seq. ID No. 3760, Seq. ID No. 3772, Seq. ID No. 3798, Seq. ID No. 3826, Seq. ID No. 3842, Seq. ID No. 3856, Seq. ID No. 3864, Seq. ID No. 3902, Seq. ID No. 3930, Seq. ID No. 4016, Seq. ID No. 4018, Seq. ID No. 4056, Seq. ID No. 4076, Seq. ID No. 4086, Seq. ID No. 4090, Seq. ID No. 4100, Seq. ID No. 4106, Seq. ID No. 4130, Seq. ID No. 4204, Seq. ID No. 4244, Seq. ID No. 4252, Seq. ID No. 4344, Seq. ID No. 4384, Seq. ID No. 4476, Seq. ID No. 4482, Seq. ID No. 4488, Seq. ID No. 4492, Seq. ID No. 4534, Seq. ID No. 4628, Seq. ID No. 4706, Seq. ID No. 4714, Seq. ID No. 4718, Seq. ID No. 4720, Seq. ID No. 4730, Seq. ID No. 4790, Seq. ID No. 4792, Seq. ID No. 4794, Seq. ID No. 4804, Seq. ID No. 4814, Seq. ID No. 4816, Seq. ID No. 4826, Seq. ID No. 4834, Seq. ID No. 4838, Seq. ID No. 4876, Seq. ID No. 4880, Seq. ID No. 4920, Seq. ID No. 4932, Seq. ID No. 4948, Seq. ID No. 4986, Seq. ID No. 5078, Seq. ID No. 5116, Seq. ID No. 5118, Seq. ID No. 5128, Seq. ID No. 5142, Seq. ID No. 5194, Seq. ID No. 5216, Seq. ID No. 5220, Seq. ID No. 5276, Seq. ID No. 5382, Seq. ID No. 5390, Seq. ID No. 5410, Seq. ID No. 5412, Seq. ID No. 5476, Seq. ID No. 5492, Seq. ID No. 5514, Seq. ID No. 5520, Seq. ID No. 5526, Seq. ID No. 5530, Seq. ID No. 5538, Seq. ID No. 5558, Seq. ID No. 5582, Seq. ID No. 5600, Seq. ID No. 5638, Seq. ID No. 5686, Seq. ID No. 5724, Seq. ID No. 5756, Seq. ID No. 5762, Seq. ID No. 5896, Seq. ID No. 5902, Seq. ID No. 5904, Seq. ID No. 5912, Seq. ID No. 5914, Seq. ID No. 5920, Seq. ID No. 5924, Seq. ID No. 5930, Seq. ID No. 6002, Seq. ID No. 6044, Seq. ID No. 6054, Seq. ID No. 6056, Seq. ID No. 6090, Seq. ID No. 6096, Seq. ID No. 6112, Seq. ID No. 6118, Seq. ID No. 6166, Seq. ID No. 6170, Seq. ID No. 6188, Seq. ID No. 6196, Seq. ID No. 6220, Seq. ID No. 6234, Seq. ID No. 6270, Seq. ID No. 6276, Seq. ID No. 6280, Seq. ID No. 6288, Seq. ID No. 6298, Seq. ID No. 6300, Seq. ID No. 6306, Seq. ID No. 6312, Seq. ID No. 6370, Seq. ID No. 6380, Seq. ID No. 6588, and Seq. ID No. 6592.

43. The Alloiococcus otitidis polypeptide of claim 36, wherein the polypeptide is identified using proteomics methodology as sharing homology with surface exposed proteins of Streptococcus pneumoniae, and is selected from one of Seq. ID No. 2, Seq. ID No. 6, Seq. ID No. 94, Seq. ID No. 96, Seq. ID No. 104, Seq. ID No. 108, Seq. ID No. 158, Seq. ID No. 224, Seq. ID No. 226, Seq. ID No. 234, Seq. ID No. 240, Seq. ID No. 254, Seq. ID No. 318, Seq. ID No. 322, Seq. ID No. 342, Seq. ID No. 346, Seq. ID No. 354, Seq. ID No. 362, Seq. ID No. 364, Seq. ID No. 366, Seq. ID No. 376, Seq. ID No. 380, Seq. ID No. 384, Seq. ID No. 390, Seq. ID No. 392, Seq. ID No. 398, Seq. ID No. 406, Seq. ID No. 410, Seq. ID No. 416, Seq. ID No. 424, Seq. ID No. 426, Seq. ID No. 428, Seq. ID No. 430, Seq. ID No. 436; Seq. ID No. 438, Seq. ID No. 440, Seq. ID No. 442, Seq. ID No. 444, Seq. ID No. 446, Seq. ID No. 448, Seq. ID No. 450, Seq. ID No. 462, Seq. ID No. 464, Seq. ID No. 466, Seq. ID No. 514, Seq. ID No. 544, Seq. ID No. 552, Seq. ID No. 554, Seq. ID No. 560, Seq. ID No. 596, Seq. ID No. 602, Seq. ID No. 638, Seq. ID No. 650, Seq. ID No. 658, Seq. ID No. 670, Seq. ID No. 676, Seq. ID No. 684, Seq. ID No. 754, Seq. ID No. 930, Seq. ID No. 970, Seq. ID No. 1042, Seq. ID No. 1044, Seq. ID No. 1086, Seq. ID No. 1090, Seq. ID No. 1094, Seq. ID No. 1098, Seq. ID No. 1100, Seq. ID No. 1102, Seq. ID No. 1176, Seq. ID No. 1206, Seq. ID No. 1238, Seq. ID No. 1460, Seq. ID No. 1496, Seq. ID No. 1498, Seq. ID No. 1504, Seq. ID No. 1518, Seq. ID No. 1528, Seq. ID No. 1532, Seq. ID No. 1540, Seq. ID No. 1544, Seq. ID No. 1550, Seq. ID No. 1574, Seq. ID No. 1658, Seq. ID No. 1710, Seq. ID No. 1768, Seq. ID No. 1786, Seq. ID No. 1822, Seq. ID No. 1890, Seq. ID No. 1926, Seq. ID No. 1970, Seq. ID No. 2004, Seq. ID No. 2048, Seq. ID No. 2076, Seq. ID No. 2090, Seq. ID No. 2094, Seq. ID No. 2118, Seq. ID No. 2138, Seq. ID No. 2144, Seq. ID No. 2206, Seq. ID No. 2236, Seq. ID No. 2258, Seq. ID No. 2302, Seq. ID No. 2364, Seq. ID No. 2424, Seq. ID No. 2446, Seq. ID No. 2452, Seq. ID No. 2482, Seq. ID No. 2496, Seq. ID No. 2506, Seq. ID No. 2578, Seq. ID No. 2588, Seq. ID No. 2614, Seq. ID No. 2634, Seq. ID No. 2652, Seq. ID No. 2666, Seq. ID No. 2670, Seq. ID No. 2678, Seq. ID No. 2688, Seq. ID No. 2694, Seq. ID No. 2700, Seq. ID No. 2712, Seq. ID No. 2716, Seq. ID No. 2758, Seq. ID No. 2792, Seq. ID No. 2794, Seq. ID No. 2804, Seq. ID No. 2868, Seq. ID No. 2920, Seq. ID No. 2930, Seq. ID No. 3006, Seq. ID No. 3020, Seq. ID No. 3126, Seq. ID No. 3138, Seq. ID No. 3162, Seq. ID No. 3180, Seq. ID No. 3252, Seq. ID No. 3260, Seq. ID No. 3286, Seq. ID No. 3310, Seq. ID No. 3318, Seq. ID No. 3338, Seq. ID No. 3346, Seq. ID No. 3360, Seq. ID No. 3476, Seq. ID No. 3516, Seq. ID No. 3522, Seq. ID No. 3642, Seq. ID No. 3696, Seq. ID No. 3794, Seq. ID No. 3902, Seq. ID No. 3928, Seq. ID No. 3988, Seq. ID No. 4002, Seq. ID No. 4058, Seq. ID No. 4082, Seq. ID No. 4112, Seq. ID No. 4126, Seq. ID No. 4194, Seq. ID No. 4196, Seq. ID No. 4238, Seq. ID No. 4248, Seq. ID No. 4384, Seq. ID No. 4394, Seq. ID No. 4398, Seq. ID No. 4402, Seq. ID No. 4436, Seq. ID No. 4450, Seq. ID No. 4472, Seq. ID No. 4492, Seq. ID No. 4512, Seq. ID No. 4520, Seq. ID No. 4532, Seq. ID No. 4550, Seq. ID No. 4578, Seq. ID No. 4582, Seq. ID No. 4586, Seq. ID No. 4610, Seq. ID No. 4612, Seq. ID No. 4614, Seq. ID No. 4676, Seq. ID No. 4686, Seq. ID No. 4720, Seq. ID No. 4730, Seq. ID No. 4744, Seq. ID No. 4756, Seq. ID No. 4782, Seq. ID No. 4788, Seq. ID No. 4838, Seq. ID No. 4842, Seq. ID No. 4850, Seq. ID No. 4856, Seq. ID No. 4890, Seq. ID No. 4902, Seq. ID No. 4916, Seq. ID No. 4918, Seq. ID No. 4932, Seq. ID No. 4936, Seq. ID No. 4978, Seq. ID No. 5026, Seq. ID No. 5030, Seq. ID No. 5110, Seq. ID No. 5116, Seq. ID No. 5118, Seq. ID No. 5156, Seq. ID No. 5186, Seq. ID No. 5202, Seq. ID No. 5204, Seq. ID No. 5220, Seq. ID No. 5262, Seq. ID No. 5290, Seq. ID No. 5296, Seq. ID No. 5378, Seq. ID No. 5390, Seq. ID No. 5400, Seq. ID No. 5412, Seq. ID No. 5538, Seq. ID No. 5622, Seq. ID No. 5628, Seq. ID No. 5658, Seq. ID No. 5676, Seq. ID No. 5718, Seq. ID No. 5778, Seq. ID No. 5860, Seq. ID No. 5902, Seq. ID No. 5914, Seq. ID No. 5920, Seq. ID No. 5926, Seq. ID No. 5934, Seq. ID No. 6056, Seq. ID No. 6130, Seq. ID No. 6134, Seq. ID No. 6188, Seq. ID No. 6194, Seq. ID No. 6220, Seq. ID No. 6272, Seq. ID No. 6278, Seq. ID No. 6370, Seq. ID No. 6376, Seq. ID No. 6380, Seq. ID No. 6390, Seq. ID No. 6592, and Seq. ID No. 6606.

44. The Alloiococcus otitidis polypeptide of claim 36, wherein the polypeptide is identified as an Alloiococcus otitidis lipoprotein, and is selected from one Seq. ID No. 46, Seq. ID No. 48, Seq. ID No. 172, Seq. ID No. 226, Seq. ID No. 420, Seq. ID No. 514, Seq. ID No. 576, Seq. ID No. 592, Seq. ID No. 594, Seq. ID No. 658, Seq. ID No. 660, Seq. ID No. 670, Seq. ID No. 676, Seq. ID No. 746, Seq. ID No. 748, Seq. ID No. 936, Seq. ID No. 1046, Seq. ID No. 1076, Seq. ID No. 1138, Seq. ID No. 1174, Seq. ID No. 1192, Seq. ID No. 1194, Seq. ID No. 1196, Seq. ID No. 1564, Seq. ID No. 1662, Seq. ID No. 1664, Seq. ID No. 1666, Seq. ID No. 1792, Seq. ID No. 1798, Seq. ID No. 1918, Seq. ID No. 1970, Seq. ID No. 2116, Seq. ID No. 2160, Seq. ID No. 2430, Seq. ID No. 2528, Seq. ID No. 2700, Seq. ID No. 2816, Seq. ID No. 2876, Seq. ID No. 2976, Seq. ID No. 2978, Seq. ID No. 2992, Seq. ID No. 3044, Seq. ID No. 3324, Seq. ID No. 3338, Seq. ID No. 4134, Seq. ID No. 4138, Seq. ID No. 4482, Seq. ID No. 4706, Seq. ID No. 4828, Seq. ID No. 4870, Seq. ID No. 5116, Seq. ID No. 5378, Seq. ID No. 5380, Seq. ID No. 5492, Seq. ID No. 5510, Seq. ID No. 5514, Seq. ID No. 5526, Seq. ID No. 5530, Seq. ID No. 5532, Seq. ID No. 5890, Seq. ID No. 5910, Seq. ID No. 6026, Seq. ID No. 6028, Seq. ID No. 6088, Seq. ID No. 6090, Seq. ID No. 6326, Seq. ID No. 6328, Seq. ID No. 6416, Seq. ID No. 6638, Seq. ID No. 6640, and Seq. ID No. 6646.

45. The Alloiococcus otitidis polypeptide of claim 36, wherein the polypeptide is identified as having a LPXTG motif and is covalently attached to the peptidoglycan layer, and is selected from one of Seq. ID No. 504, Seq. ID No. 506, Seq. ID No. 1316, Seq. ID No. 1318, Seq. ID No. 1364, Seq. ID No. 1366, Seq. ID No. 1368, Seq. ID No. 1370, Seq. ID No. 1560, Seq. ID No. 1562, Seq. ID No. 1582, Seq. ID No. 1740, Seq. ID No. 1742, Seq. ID No. 2812, Seq. ID No. 2814, Seq. ID No. 3620, Seq. ID No. 5588, Seq. ID No. 5590, Seq. ID No. 5592, Seq. ID No. 5782, Seq. ID No. 5784, and Seq. ID No. 6104.

46. The Alloiococcus otitidis polypeptide of claim 36, wherein the polypeptide is identified as an Alloiococcus otitidis polypeptide, which is non-covalently associated with the peptidoglycan layer, and is selected from one of Seq. ID No. 3590, Seq. ID No. 3988, Seq. ID No. 5220, and Seq. ID No. 5338.

47. The Alloiococcus otitidis polypeptide of claim 36, wherein the polypeptide is identified as an Alloiococcus otitidis polypeptide having an RGDX motif wherein X is not a proline residue and is selected from one of Seq. ID No. 38, Seq. ID No. 100, Seq. ID No. 108, Seq. ID No. 110, Seq. ID No. 112, Seq. ID No. 352, Seq. ID No. 354, Seq. ID No. 466, Seq. ID No. 468, Seq. ID No. 470, Seq. ID No. 472, Seq. ID No. 684, Seq. ID No. 822, Seq. ID No. 824, Seq. ID No. 1022, Seq. ID No. 1024, Seq. ID No. 1158, Seq. ID No. 1160, Seq. ID No. 1234, Seq. ID No. 1334, Seq. ID No. 1336, Seq. ID No. 1450, Seq. ID No. 1560, Seq. ID No. 1562, Seq. ID No. 1564, Seq. ID No. 1626, Seq. ID No. 1778, Seq. ID No. 1828, Seq. ID No. 1830, Seq. ID No. 1872, Seq. ID No. 1926, Seq. ID No. 2136, Seq. ID No. 2146, Seq. ID No. 2174, Seq. ID No. 2176, Seq. ID No. 2178, Seq. ID No. 2180, Seq. ID No. 2260, Seq. ID No. 2318, Seq. ID No. 2320, Seq. ID No. 2322, Seq. ID No. 2324, Seq. ID No. 2326, Seq. ID No. 2328, Seq. ID No. 2330, Seq. ID No. 2402, Seq. ID No. 2672, Seq. ID No. 2864, Seq. ID No. 2870, Seq. ID No. 2920, Seq. ID No. 2922, Seq. ID No. 2994, Seq. ID No. 3088, Seq. ID No. 3138, Seq. ID No. 3280, Seq. ID No. 3282, Seq. ID No. 3284, Seq. ID No. 3334, Seq. ID No. 3336, Seq. ID No. 3498, Seq. ID No. 3500, Seq. ID No. 3502, Seq. ID No. 3534, Seq. ID No. 3672, Seq. ID No. 3674, Seq. ID No. 3676, Seq. ID No. 3838, Seq. ID No. 3840, Seq. ID No. 3842, Seq. ID No. 3844, Seq. ID No. 3930, Seq. ID No. 3962, Seq. ID No. 4038, Seq. ID No. 4194, Seq. ID No. 4240, Seq. ID No. 4328, Seq. ID No. 4330, Seq. ID No. 4334, Seq. ID No. 4394, Seq. ID No. 4464, Seq. ID No. 4466, Seq. ID No. 4468, Seq. ID No. 4598, Seq. ID No. 4630, Seq. ID No. 4632, Seq. ID No. 4676, Seq. ID No. 4678, Seq. ID No. 4680, Seq. ID No. 4690, Seq. ID No. 4692, Seq. ID No. 4694, Seq. ID No. 4782, Seq. ID No. 4800, Seq. ID No. 4802, Seq. ID No. 4804, Seq. ID No. 4862, Seq. ID No. 5010, Seq. ID No. 5144, Seq; ID No. 5146, Seq. ID No. 5180, Seq. ID No. 5182, Seq. ID No. 5184, Seq. ID No. 5250, Seq. ID No. 5252, Seq. ID No. 5254, Seq. ID No. 5260, Seq. ID No. 5262, Seq. ID No. 5294, Seq. ID No. 5296, Seq. ID No. 5298, Seq. ID No. 5300, Seq. ID No. 5308, Seq. ID No. 5310, Seq. ID No. 5412, Seq. ID No. 5536, Seq. ID No. 5538, Seq. ID No. 5746, Seq. ID No. 5822, Seq. ID No. 5824, Seq. ID No. 5826, Seq. ID No. 6030, Seq. ID No. 6172, Seq. ID No. 6308, Seq. ID No. 6310, and Seq. ID No. 6312.

48. The Alloiococcus otitidis polypeptide of claim 36, wherein the polypeptide is predicted by BlastP as being involved in capsule biosynthesis and transport, and is selected from one of Seq. ID No. 50, Seq. ID No. 52, Seq. ID No. 54, Seq. ID No. 60, Seq. ID No. 196, Seq. ID No. 690, Seq. ID No. 704, Seq. ID No. 926, Seq. ID No. 1598, Seq. ID No. 1602, Seq. ID No. 1608, Seq. ID No. 1612, Seq. ID No. 1614, Seq. ID No. 1714, Seq. ID No. 2030, Seq. ID No. 2264, Seq. ID No. 2270, Seq. ID No. 2374, Seq. ID No. 2438, Seq. ID No. 2652, Seq. ID No. 2768, Seq. ID No. 2908, Seq. ID No. 3398, Seq. ID No. 3400, Seq. ID No. 3480, Seq. ID No. 3488, Seq. ID No. 3492, Seq. ID No. 3494, Seq. ID No. 3496, Seq. ID No. 3498, Seq. ID No. 3520, Seq. ID No. 3522, Seq. ID No. 3530, Seq. ID No. 3536, Seq. ID No. 3562, Seq. ID No. 3884, Seq. ID No. 3892, Seq. ID No. 3982, and Seq. ID No. 4088.

49. The Alloiococcus otitidis polypeptide of claim 36 wherein the polynucleotide encoding the polypeptide is predicted by BlastP as being localized in the capsule loci region of Alloiococcus otitidis, and is selected from one of Seq. ID No. 46, Seq. ID No. 48, Seq. ID No. 52, Seq. ID No. 54, Seq. ID No. 56, Seq. ID No. 58, Seq. ID No. 60, Seq. ID No. 684, Seq. ID No. 686, Seq. ID No. 688, Seq. ID No. 690, Seq. ID No. 692, Seq. ID No. 694, Seq. ID No. 696, Seq. ID No. 698, Seq. ID No. 700, Seq. ID No. 702, Seq. ID No. 704, Seq. ID No. 706, Seq. ID No. 708, Seq. ID No. 710, Seq. ID No. 712, Seq. ID No. 714, Seq. ID No. 716, Seq. ID No. 718, Seq. ID No. 720, Seq. ID No. 722, Seq. ID No. 724, Seq. ID No. 726, Seq. ID No. 728, Seq. ID No. 730, Seq. ID No. 732, Seq. ID No. 734, Seq. ID No. 736, Seq. ID No. 738, Seq. ID No. 740, Seq. ID No. 742, Seq. ID No. 744, Seq. ID No. 746, Seq. ID No. 748, Seq. ID No. 750, Seq. ID No. 752, Seq. ID No. 754, Seq. ID No. 3390, Seq. ID No. 3392, Seq. ID No. 3394, Seq. ID No. 3396, Seq. ID No. 3398, Seq. ID No. 3400, Seq. ID No. 3402, Seq. ID No. 3404, Seq. ID No. 3406, Seq. ID No. 3408, Seq. ID No. 3410, Seq. ID No. 3412, Seq. ID No. 3414, Seq. ID No. 3416, Seq. ID No. 3418, Seq. ID No. 3420, Seq. ID No. 3422, Seq. ID No. 3424, Seq. ID No. 3426, Seq. ID No. 3428, Seq. ID No. 3430, Seq. ID No. 3432, Seq. ID No. 3434, Seq. ID No. 3436, Seq. ID No. 3438, Seq. ID No. 3440, Seq. ID No. 3442, Seq. ID No. 3444, Seq. ID No. 3446, Seq. ID No. 3448, Seq. ID No. 3450, Seq. ID No. 3452, Seq. ID No. 3454, Seq. ID No. 3456, Seq. ID No. 3458, Seq. ID No. 3460, Seq. ID No. 3462, Seq. ID No. 3464, Seq. ID No. 3466, Seq. ID No. 3468, Seq. ID No. 3470, Seq. ID No. 3472, Seq. ID No. 3474, Seq. ID No. 3476, Seq. ID No. 3478, Seq. ID No. 3480, Seq. ID No. 3482, Seq. ID No. 3484, Seq. ID No. 3486, Seq. ID No. 3488, Seq. ID No. 3490, Seq. ID No. 3492, Seq. ID No. 3494, Seq. ID No. 3496, Seq. ID No. 3498, Seq. ID No. 3500, Seq. ID No. 3502, Seq. ID No. 3504, Seq. ID No. 3506, Seq. ID No. 3508, Seq. ID No. 3510, Seq. ID No. 3512, Seq. ID No. 3514, Seq. ID No. 3516, Seq. ID No. 3518, Seq. ID No. 3520, Seq. ID No. 3522, Seq. ID No. 3524, Seq. ID No. 3526, Seq. ID No. 3528, Seq. ID No. 3530, Seq. ID No. 3532, Seq. ID No. 3534, Seq. ID No. 3536, Seq. ID No. 3538, Seq. ID No. 3540, Seq. ID No. 3542, Seq. ID No. 3544, Seq. ID No. 3546, Seq. ID No. 3548, Seq. ID No. 3550, Seq. ID No. 3552, Seq. ID No. 3554, Seq. ID No. 3556, Seq. ID No. 3558, Seq. ID No. 3560, and Seq. ID No. 3562.

50. The Alloiococcus otitidis polypeptide of claim 36, wherein the polypeptide is predicted by BlastP to be associated with sporulation, and is selected from one of Seq. ID No. 1138, Seq. ID No. 1456, Seq. ID No. 2138, Seq. ID No. 2142, Seq. ID No. 2166, Seq. ID No. 2180, Seq. ID No. 2302, Seq. ID No. 2754, Seq. ID No. 4212, Seq. ID No. 4252, Seq. ID No. 4328, Seq. ID No. 4570, Seq. ID No. 4720, and Seq. ID No. 4730.

51. The Alloiococcus otitidis polypeptide of claim 36 wherein the polypeptide encoded by the polynucleotide is predicted by BlastP to be a unique polypeptide, and is selected from one of Seq. ID No. 8, Seq. ID No. 20, Seq. ID No. 22, Seq. ID No. 24, Seq. ID No. 32, Seq. ID No. 40, Seq. ID No. 66, Seq. ID No. 68, Seq. ID No. 70, Seq. ID No. 92, Seq. ID No. 100, Seq. ID No. 106, Seq. ID No. 114, Seq. ID No. 116, Seq. ID No. 124, Seq. ID No. 126, Seq. ID No. 128, Seq. ID No. 132, Seq. ID No. 134, Seq. ID No. 140, Seq. ID No. 152, Seq. ID No. 160, Seq. ID No. 162, Seq. ID No. 164, Seq. ID No. 166, Seq. ID No. 170, Seq. ID No. 186, Seq. ID No. 190, Seq. ID No. 194, Seq. ID No. 230, Seq. ID No. 232, Seq. ID No. 256, Seq. ID No. 258, Seq. ID No. 260, Seq. ID No. 276, Seq. ID No. 338, Seq. ID No. 340, Seq. ID No. 348, Seq. ID No. 350, Seq. ID No. 358, Seq. ID No. 360, Seq. ID No. 370, Seq. ID No. 372, Seq. ID No. 378, Seq. ID No. 386, Seq. ID No. 388, Seq. ID No. 394, Seq. ID No. 400, Seq. ID No. 402, Seq. ID No. 404, Seq. ID No. 408, Seq. ID No. 420, Seq. ID No. 432, Seq. ID No. 478, Seq. ID No. 496, Seq. ID No. 508, Seq. ID No. 520, Seq. ID No. 542, Seq. ID No. 546, Seq. ID No. 548, Seq. ID No. 550, Seq. ID No. 558, Seq. ID No. 590, Seq. ID No. 618, Seq. ID No. 620, Seq. ID No. 634, Seq. ID No. 640, Seq. ID No. 642, Seq. ID No. 644, Seq. ID No. 646, Seq. ID No. 648, Seq. ID No. 652, Seq. ID No. 654, Seq. ID No. 656, Seq. ID No. 668, Seq. ID No. 692, Seq. ID No. 694, Seq. ID No. 696, Seq. ID No. 698, Seq. ID No. 716, Seq. ID No. 718, Seq. ID No. 720, Seq. ID No. 732, Seq. ID No. 734, Seq. ID No. 742, Seq. ID No. 744, Seq. ID No. 746, Seq. ID No. 748, Seq. ID No. 750, Seq. ID No. 752, Seq. ID No. 756, Seq. ID No. 758, Seq. ID No. 760, Seq. ID No. 762, Seq. ID No. 764, Seq. ID No. 766, Seq. ID No. 768, Seq. ID No. 802, Seq. ID No. 804, Seq. ID No. 806, Seq. ID No. 808, Seq. ID No. 810, Seq. ID No. 812, Seq. ID No. 814, Seq. ID No. 834, Seq. ID No. 842, Seq. ID No. 850, Seq. ID No. 852, Seq. ID No. 854, Seq. ID No. 856, Seq. ID No. 888, Seq. ID No. 890, Seq. ID No. 892, Seq. ID No. 904, Seq. ID No. 906, Seq. ID No. 910, Seq. ID No. 938, Seq. ID No. 942, Seq. ID No. 944, Seq. ID No. 952, Seq. ID No. 958, Seq. ID No. 974, Seq. ID No. 976, Seq. ID No. 988, Seq. ID No. 1006, Seq. ID No. 1008, Seq. ID No. 1010, Seq: ID No. 1012, Seq. ID No. 1020, Seq. ID No. 1026, Seq. ID No. 1028, Seq. ID No. 1046, Seq. ID No. 1048, Seq. ID No. 1072, Seq. ID No. 1074, Seq. ID No. 1078, Seq. ID No. 1106, Seq. ID No. 1108, Seq. ID No. 1112, Seq. ID No. 1132, Seq. ID No. 1136, Seq. ID No. 1140, Seq. ID No. 1152, Seq. ID No. 1154, Seq. ID No. 1166, Seq. ID No. 1168, Seq. ID No. 1170, Seq. ID No. 1172, Seq. ID No. 1174, Seq. ID No. 1182, Seq. ID No. 1184, Seq. ID No. 1186, Seq. ID No. 1188, Seq. ID No. 1190, Seq. ID No. 1192, Seq. ID No. 1194, Seq. ID No. 1196, Seq. ID No. 1198, Seq. ID No. 1200, Seq. ID No. 1202, Seq. ID No. 1204, Seq. ID No. 1208, Seq. ID No. 1214, Seq. ID No. 1228, Seq. ID No. 1230, Seq. ID No. 1240, Seq. ID No. 1244, Seq. ID No. 1246, Seq. ID No. 1248, Seq. ID No. 1250, Seq. ID No. 1260, Seq. ID No. 1266, Seq. ID No. 1268, Seq. ID No. 1270, Seq. ID No. 1272, Seq. ID No. 1274, Seq. ID No. 1276, Seq. ID No. 1278, Seq. ID No. 1280, Seq. ID No. 1282, Seq. ID No. 1284, Seq. ID No. 1286, Seq. ID No. 1288, Seq. ID No. 1290, Seq. ID No. 1292, Seq. ID No. 1294, Seq. ID No. 1296, Seq. ID No. 1298, Seq. ID No. 1300, Seq. ID No. 1302, Seq. ID No. 1304, Seq. ID No. 1306, Seq. ID No. 1308, Seq. ID No. 1310, Seq. ID No. 1312, Seq. ID No. 1314, Seq. ID No. 1320, Seq. ID No. 1322, Seq. ID No. 1324, Seq. ID No. 1326, Seq. ID No. 1328, Seq. ID No. 1338, Seq. ID No. 1342, Seq. ID No. 1344, Seq. ID No. 1348, Seq. ID No. 1350, Seq. ID No. 1352, Seq. ID No. 1354, Seq. ID No. 1356, Seq. ID No. 1372, Seq. ID No. 1380, Seq. ID No. 1382, Seq. ID No. 1390, Seq. ID No. 1398, Seq. ID No. 1400, Seq. ID No. 1402, Seq. ID No. 1418, Seq. ID No. 1420, Seq. ID No. 1422, Seq. ID No. 1424, Seq. ID No. 1440, Seq. ID No. 1446, Seq. ID No. 1448, Seq. ID No. 1458, Seq. ID No. 1466, Seq. ID No. 1480, Seq. ID No. 1512, Seq. ID No. 1552, Seq. ID No. 1554, Seq. ID No. 1556, Seq. ID No. 1558, Seq. ID No. 1580, Seq. ID No. 1610, Seq. ID No. 1622, Seq. ID No. 1624, Seq. ID No. 1626, Seq. ID No. 1628, Seq. ID No. 1630, Seq. ID No. 1632, Seq. ID No. 1634, Seq. ID No. 1640, Seq. ID No. 1644, Seq. ID No. 1656, Seq. ID No. 1668, Seq. ID No. 1672, Seq. ID No. 1694, Seq. ID No. 1696, Seq. ID No. 1702, Seq. ID No. 1704, Seq. ID No. 1706, Seq. ID No. 1722, Seq. ID No. 1724, Seq. ID No. 1732, Seq. ID No. 1738, Seq. ID No. 1744, Seq. ID No. 1760, Seq. ID No. 1764, Seq. ID No. 1774, Seq. ID No. 1776, Seq. ID No. 1778, Seq. ID No. 1780, Seq. ID No. 1782, Seq. ID No. 1800, Seq. ID No. 1802, Seq. ID No. 1804, Seq. ID No. 1808, Seq. ID No. 1824, Seq. ID No. 1832, Seq. ID No. 1852, Seq. ID No. 1858, Seq. ID No. 1876, Seq. ID No. 1900, Seq. ID No. 1916, Seq. ID No. 1928, Seq. ID No. 1930, Seq. ID No. 1974, Seq. ID No. 1978, Seq. ID No. 1982, Seq. ID No. 2006, Seq. ID No. 2008, Seq. ID No. 2010, Seq. ID No. 2012, Seq. ID No. 2018, Seq. ID No. 2020, Seq. ID No. 2024, Seq. ID No. 2026, Seq. ID No. 2028, Seq. ID No. 2040, Seq. ID No. 2042, Seq. ID No. 2044, Seq. ID No. 2046, Seq. ID No. 2050, Seq. ID No. 2082, Seq. ID No. 2084, Seq. ID No. 2086, Seq. ID No. 2106, Seq. ID No. 2108, Seq. ID No. 2110, Seq. ID No. 2112, Seq. ID No. 2130, Seq. ID No. 2132, Seq. ID No. 2134, Seq. ID No. 2146, Seq. ID No. 2156, Seq. ID No. 2158, Seq. ID No. 2160, Seq. ID No. 2162, Seq. ID No. 2172, Seq. ID No. 2188, Seq. ID No. 2190, Seq. ID No. 2202, Seq. ID No. 2204, Seq. ID No. 2210, Seq. ID No. 2212, Seq. ID No. 2920, Seq. ID No. 2226, Seq. ID No. 2244, Seq. ID No. 2246, Seq. ID No. 2248, Seq. ID No. 2250, Seq. ID No. 2252, Seq. ID No. 2256, Seq. ID No. 2278, Seq. ID No. 2280, Seq. ID No. 2342, Seq. ID No. 2352, Seq. ID No. 2354, Seq. ID No. 2356, Seq. ID No. 2358, Seq. ID No. 2360, Seq. ID No. 2366, Seq. ID No. 2368, Seq. ID No. 2372, Seq. ID No. 2376, Seq. ID No. 2394, Seq. ID No. 2396, Seq. ID No. 2426, Seq. ID No. 2428, Seq. ID No. 2432, Seq. ID No. 2440, Seq. ID No. 2442, Seq. ID No. 2444, Seq. ID No. 2448, Seq. ID No. 2460, Seq. ID No. 2480, Seq. ID No. 2494, Seq. ID No. 2502, Seq. ID No. 2504, Seq. ID No. 2522, Seq. ID No. 2524, Seq. ID No. 2528, Seq. ID No. 2592, Seq. ID No. 2602, Seq. ID No. 2604, Seq. ID No. 2612, Seq. ID No. 2628, Seq. ID No. 2630, Seq. ID No. 2654, Seq. ID No. 2662, Seq. ID No. 2692, Seq. ID No. 2696, Seq. ID No. 2698, Seq. ID No. 2718, Seq. ID No. 2730, Seq. ID No. 2732, Seq. ID No. 2734, Seq. ID No. 2736, Seq. ID No. 2740, Seq. ID No. 2742, Seq. ID No. 2744, Seq. ID No. 2778, Seq. ID No. 2780, Seq. ID No. 2782, Seq. ID No. 2784, Seq. ID No. 2802, Seq. ID No. 2806, Seq. ID No. 2808, Seq. ID No. 2810, Seq. ID No. 2812, Seq. ID No. 2814, Seq. ID No. 2816, Seq. ID No. 2818, Seq. ID No. 2820, Seq. ID No. 2830, Seq. ID No. 2832, Seq. ID No. 2834, Seq. ID No. 2836, Seq. ID No. 2838, Seq. ID No. 2840, Seq. ID No. 2842, Seq. ID No. 2846, Seq. ID No. 2850, Seq. ID No. 2852, Seq. ID No. 2854, Seq. ID No. 2856, Seq. ID No. 2858, Seq. ID No. 2860, Seq. ID No. 2862, Seq. ID No. 2866, Seq. ID No. 2874, Seq. ID No. 2876, Seq. ID No. 2878, Seq. ID No. 2884, Seq. ID No. 2886, Seq. ID No. 2888, Seq. ID No. 2890, Seq. ID No. 2894, Seq. ID No. 2896, Seq. ID No. 2904, Seq. ID No. 2910, Seq. ID No. 2936, Seq. ID No. 2938, Seq. ID No. 2940, Seq. ID No. 2942, Seq. ID No. 2944, Seq. ID No. 2946, Seq. ID No. 2948, Seq. ID No. 2950, Seq. ID No. 2952, Seq. ID No. 2954, Seq. ID No. 2966, Seq. ID No. 2968, Seq. ID No. 2974, Seq. ID No. 2976, Seq. ID No. 2978, Seq. ID No. 2982, Seq. ID No. 2984, Seq. ID No. 2986, Seq. ID No. 2988, Seq. ID No. 2990, Seq. ID No. 2992, Seq. ID No. 3002, Seq. ID No. 3008, Seq. ID No. 3010, Seq. ID No. 3012, Seq. ID No. 3022, Seq. ID No. 3024, Seq. ID No. 3042, Seq. ID No. 3046, Seq. ID No. 3072, Seq. ID No. 3088, Seq. ID No. 3106, Seq. ID No. 3108, Seq. ID No. 3120, Seq. ID No. 3122, Seq. ID No. 3124, Seq. ID No. 3152, Seq. ID No. 3154, Seq. ID No. 3168, Seq. ID No. 3174, Seq. ID No. 3176, Seq. ID No. 3178, Seq. ID No. 3182, Seq. ID No. 3194, Seq. ID No. 3214, Seq. ID No. 3216, Seq. ID No. 3218, Seq. ID No. 3220, Seq. ID No. 3222, Seq. ID No. 3224, Seq. ID No. 3232, Seq. ID No. 3234, Seq. ID No. 3236, Seq. ID No. 3238, Seq. ID No. 3240, Seq. ID No. 3242, Seq. ID No. 3244, Seq. ID No. 3246, Seq. ID No. 3248, Seq. ID No. 3250, Seq. ID No. 3262, Seq. ID No. 3264, Seq. ID No. 3266, Seq. ID No. 3268, Seq. ID No. 3270, Seq. ID No. 3272, Seq. ID No. 3274, Seq. ID No. 3276, Seq. ID No. 3278, Seq. ID No. 3326, Seq. ID No. 3332, Seq. ID No. 3340, Seq. ID No. 3342, Seq. ID No. 3350, Seq. ID No. 3362, Seq. ID No. 3364, Seq. ID No. 3370, Seq. ID No. 3372, Seq. ID No. 3390, Seq. ID No. 3404, Seq. ID No. 3418, Seq. ID No. 3420, Seq. ID No. 3422, Seq. ID No. 3434, Seq. ID No. 3438, Seq. ID No. 3442, Seq. ID No. 3444, Seq. ID No. 3446, Seq. ID No. 3448, Seq. ID No. 3474, Seq. ID No. 3482, Seq. ID No. 3484, Seq. ID No. 3490, Seq. ID No. 3504, Seq. ID No. 3506, Seq. ID No. 3510, Seq. ID No. 3512, Seq. ID No. 3524, Seq. ID No. 3526, Seq. ID No. 3532, Seq. ID No. 3534, Seq. ID No. 3540, Seq. ID No. 3542, Seq. ID No. 3544, Seq. ID No. 3546, Seq. ID No. 3548, Seq. ID No. 3556, Seq. ID No. 3558, Seq. ID No. 3564, Seq. ID No. 3566, Seq. ID No. 3572, Seq. ID No. 3574, Seq. ID No. 3582, Seq. ID No. 3588, Seq. ID No. 3592, Seq. ID No. 3594, Seq. ID No. 3596, Seq. ID No. 3612, Seq. ID No. 3614, Seq. ID No. 3618, Seq. ID No. 3620, Seq. ID No. 3622, Seq. ID No. 3624, Seq. ID No. 3626, Seq. ID No. 3632, Seq. ID No. 3638, Seq. ID No. 3640, Seq. ID No. 3656, Seq. ID No. 3658, Seq. ID No. 3660, Seq. ID No. 3662, Seq. ID No. 3678, Seq. ID No. 3680, Seq. ID No. 3684, Seq. ID No. 3686, Seq. ID No. 3702, Seq. ID No. 3704, Seq. ID No. 3718, Seq. ID No. 3720, Seq. ID No. 3722, Seq. ID No. 3724, Seq. ID No. 3730, Seq. ID No. 3732, Seq. ID No. 3734, Seq. ID No. 3736, Seq. ID No. 3738, Seq. ID No. 3740, Seq. ID No. 3742, Seq. ID No. 3750, Seq. ID No. 3752, Seq. ID No. 3754, Seq. ID No. 3756, Seq. ID No. 3762, Seq. ID No. 3782, Seq. ID No. 3784, Seq. ID No. 3786, Seq. ID No. 3788, Seq. ID No. 3790, Seq. ID No. 3792, Seq. ID No. 3800, Seq. ID No. 3808, Seq. ID No. 3810, Seq. ID No. 3812, Seq. ID No. 3814, Seq. ID No. 3816, Seq. ID No. 3818, Seq. ID No. 3828, Seq. ID No. 3830, Seq. ID No. 3832, Seq. ID No. 3834, Seq. ID No. 3836, Seq. ID No. 3850, Seq. ID No. 3880, Seq. ID No. 3882, Seq. ID No. 3896, Seq. ID No. 3904, Seq. ID No. 3910, Seq. ID No. 3916, Seq. ID No. 3940, Seq. ID No. 3960, Seq. ID No. 3962, Seq. ID No. 3964, Seq. ID No. 3966, Seq. ID No. 3974, Seq. ID No. 3978, Seq. ID No. 3980, Seq. ID No. 3994, Seq. ID No. 3996, Seq. ID No. 3998, Seq. ID No. 4006, Seq: ID No. 4008, Seq. ID No. 4010, Seq. ID No. 4042, Seq. ID No. 4044, Seq. ID No. 4046, Seq. ID No. 4070, Seq. ID No. 4078, Seq. ID No. 4098, Seq. ID No. 4114, Seq. ID No. 4124, Seq. ID No. 4132, Seq. ID No. 4134, Seq. ID No. 4136, Seq. ID No. 4140, Seq. ID No. 4152, Seq. ID No. 4154, Seq. ID No. 4156, Seq. ID No. 4182, Seq. ID No. 4222, Seq. ID No. 4242, Seq. ID No. 4254, Seq. ID No. 4258, Seq. ID No. 4260, Seq. ID No. 4262, Seq. ID No. 4264, Seq. ID No. 4266, Seq. ID No. 4270, Seq. ID No. 4276, Seq. ID No. 4278, Seq. ID No. 4280, Seq. ID No. 4282, Seq. ID No. 4286, Seq. ID No. 4288, Seq. ID No. 4298, Seq. ID No. 4306, Seq. ID No. 4308, Seq. ID No. 4364, Seq. ID No. 4366, Seq. ID No. 4372, Seq. ID No. 4374, Seq. ID No. 4396, Seq. ID No. 4400; Seq. ID No. 4404, Seq. ID No. 4426, Seq. ID No. 4438, Seq. ID No. 4440, Seq. ID No. 4442, Seq. ID No. 4454, Seq. ID No. 4456, Seq. ID No. 4514, Seq. ID No. 4516, Seq. ID No. 4552, Seq. ID No. 4562, Seq. ID No. 4574, Seq. ID No. 4576, Seq. ID No. 4588, Seq. ID No. 4590, Seq. ID No. 4592, Seq. ID No. 4594, Seq. ID No. 4596, Seq. ID No. 4618, Seq. ID No. 4620, Seq. ID No. 4622, Seq. ID No. 4626, Seq. ID No. 4628, Seq. ID No. 4634, Seq. ID No. 4636, Seq. ID No. 4638, Seq. ID No. 4640, Seq. ID No. 4660, Seq. ID No. 4682, Seq. ID No. 4688, Seq. ID No. 4696, Seq. ID No. 4700, Seq. ID No. 4702, Seq. ID No. 4722, Seq. ID No. 4736, Seq. ID No. 4738, Seq. ID No. 4742, Seq. ID No. 4784, Seq. ID No. 4790, Seq. ID No. 4806, Seq. ID No. 4812, Seq. ID No. 4818, Seq. ID No. 4820, Seq. ID No. 4822, Seq. ID No. 4824, Seq. ID No. 4836, Seq. ID No. 4854, Seq. ID No. 4886, Seq. ID No. 4914, Seq. ID No. 4924, Seq. ID No. 4950, Seq. ID No. 4952, Seq. ID No. 4976, Seq. ID No. 4982, Seq. ID No. 4984, Seq. ID No. 4988, Seq. ID No. 4994, Seq. ID No. 5000, Seq. ID No. 5002, Seq. ID No. 5012, Seq. ID No. 5014, Seq. ID No. 5024, Seq. ID No. 5032, Seq. ID No. 5034, Seq. ID No. 5036, Seq. ID No. 5038, Seq. ID No. 5042, Seq. ID No. 5044, Seq. ID No. 5054, Seq. ID No. 5062, Seq. ID No. 5064, Seq. ID No. 5086, Seq. ID No. 5094, Seq. ID No. 5152, Seq. ID No. 5154, Seq. ID No. 5160, Seq. ID No. 5162, Seq. ID No. 5168, Seq. ID No. 5174, Seq. ID No. 5176, Seq. ID No. 5178, Seq. ID No. 5190, Seq. ID No. 5192, Seq. ID No. 5194, Seq. ID No. 5198, Seq. ID No. 5200, Seq. ID No. 5222, Seq. ID No. 5224, Seq. ID No. 5226, Seq. ID No. 5228, Seq. ID No. 5238, Seq. ID No. 5268, Seq. ID No. 5270, Seq. ID No. 5272, Seq. ID No. 5282, Seq. ID No. 5302, Seq. ID No. 5304, Seq. ID No. 5316, Seq. ID No. 5322, Seq. ID No. 5328, Seq. ID No. 5348, Seq. ID No. 5386, Seq. ID No. 5446, Seq. ID No. 5448, Seq. ID No. 5460, Seq. ID No. 5466, Seq. ID No. 5468, Seq. ID No. 5480, Seq. ID No. 5482, Seq. ID No. 5486, Seq. ID No. 5488, Seq. ID No. 5516, Seq. ID No. 5518, Seq. ID No. 5534, Seq. ID No. 5544, Seq. ID No. 5546, Seq. ID No. 5550, Seq. ID No. 5570, Seq. ID No. 5578, Seq. ID No. 5594, Seq. ID No. 5596, Seq. ID No. 5610, Seq. ID No. 5624, Seq. ID No. 5626, Seq. ID No. 5640, Seq. ID No. 5642, Seq. ID No. 5644, Seq. ID No. 5650, Seq. ID No. 5660, Seq. ID No. 5662, Seq. ID No. 5664, Seq. ID No. 5668, Seq. ID No. 5672, Seq. ID No. 5688, Seq. ID No. 5690, Seq. ID No. 5704, Seq. ID No. 5706, Seq. ID No. 5710, Seq. ID No. 5712, Seq. ID No. 5738, Seq. ID No. 5740, Seq. ID No. 5746, Seq. ID No. 5752, Seq. ID No. 5764, Seq. ID No. 5798, Seq. ID No. 5812, Seq. ID No. 5814, Seq. ID No. 5816, Seq. ID No. 5828, Seq. ID No. 5830, Seq. ID No. 5844, Seq. ID No. 5846, Seq. ID No. 5874, Seq. ID No. 5880, Seq. ID No. 5884, Seq. ID No. 5886, Seq. ID No. 5888, Seq. ID No. 5890, Seq. ID No. 5892, Seq. ID No. 5932, Seq. ID No. 5956, Seq. ID No. 5964, Seq. ID No. 5966, Seq. ID No. 5976, Seq. ID No. 5984, Seq. ID No. 5986, Seq. ID No. 5988, Seq. ID No. 5990, Seq. ID No. 6006, Seq. ID No. 6014, Seq. ID No. 6032, Seq. ID No. 6036, Seq. ID No. 6046, Seq. ID No. 6060, Seq. ID No. 6062, Seq. ID No. 6064, Seq. ID No. 6074, Seq. ID No. 6082, Seq. ID No. 6092, Seq. ID No. 6094, Seq. ID No. 6128, Seq. ID No. 6160, Seq. ID No. 6198, Seq. ID No. 6200, Seq. ID No. 6204, Seq. ID No. 6218, Seq. ID No. 6222, Seq. ID No. 6224, Seq. ID No. 6238, Seq. ID No. 6240, Seq. ID No. 6242, Seq. ID No. 6246, Seq. ID No. 6248, Seq. ID No. 6250, Seq. ID No. 6260, Seq. ID No. 6266, Seq. ID No. 6268, Seq. ID No. 6274, Seq. ID No. 6284, Seq. ID No. 6286, Seq. ID No. 6324, Seq. ID No. 6326, Seq. ID No. 6328, Seq. ID No. 6362, Seq. ID No. 6364, Seq. ID No. 6378, Seq. ID No. 6382, Seq. ID No. 6402, Seq. ID No. 6404, Seq. ID No. 6420, Seq. ID No. 6426, Seq. ID No. 6428, Seq. ID No. 6430, Seq. ID No. 6432, Seq. ID No. 6434, Seq. ID No. 6436, Seq. ID No. 6438, Seq. ID No. 6440, Seq. ID No. 6448, Seq. ID No. 6450, Seq. ID No. 6452, Seq. ID No. 6454, Seq. ID No. 6456, Seq. ID No. 6458, Seq. ID No. 6466, Seq. ID No. 6468, Seq. ID No. 6470, Seq. ID No. 6472, Seq. ID No. 6474, Seq. ID No. 6476, Seq. ID No. 6478, Seq. ID No. 6480, Seq. ID No. 6482, Seq. ID No. 6484, Seq. ID No. 6486, Seq. ID No. 6488, Seq. ID No. 6490, Seq. ID No. 6492, Seq. ID No. 6496, Seq. ID No. 6498, Seq. ID No. 6508, Seq. ID No. 6518, Seq. ID No. 6520, Seq. ID No. 6522, Seq. ID No. 6524, Seq. ID No. 6526, Seq. ID No. 6528, Seq. ID No. 6530, Seq. ID No. 6534, Seq. ID No. 6540, Seq. ID No. 6542, Seq. ID No. 6544, Seq. ID No. 6556, Seq. ID No. 6558, Seq. ID No. 6562, Seq. ID No. 6564, Seq. ID No. 6566, Seq. ID No. 6568, Seq. ID No. 6570, Seq. ID No. 6572, Seq. ID No. 6574, Seq. ID No. 6582, Seq. ID No. 6584, Seq. ID No. 6632, Seq. ID No. 6636, Seq. ID No. 6638, Seq. ID No. 6640, Seq. ID No. 6644, Seq. ID No. 6646, Seq. ID No. 6648, and Seq. ID No. 6650.

52. The Alloiococcus otitidis polypeptide of claim 36, wherein the polypeptide is identified as an Alloiococcus otitidis polypeptide identified by Glimmer™ ORF finder program, and is selected from one of Seq. ID No. 2, Seq. ID No. 6, Seq. ID No. 12, Seq. ID No. 14, Seq. ID No. 16, Seq. ID No. 18, Seq. ID No. 26, Seq. ID No. 28, Seq. ID No. 30, Seq. ID No. 36, Seq. ID No. 38, Seq. ID No. 42, Seq. ID No. 44, Seq. ID No. 48, Seq. ID No. 52, Seq. ID No. 54, Seq. ID No. 60, Seq. ID No. 64, Seq. ID No. 70, Seq. ID No. 74, Seq. ID No. 76, Seq. ID No. 82, Seq. ID No. 84, Seq. ID No. 90, Seq. ID No. 94, Seq. ID No. 98, Seq. ID No. 104, Seq. ID No. 112, Seq. ID No. 122, Seq. ID No. 126, Seq. ID No. 130, Seq. ID No. 136, Seq. ID No. 138, Seq. ID No. 146, Seq. ID No. 150, Seq. ID No. 158, Seq. ID No. 168, Seq. ID No. 174, Seq. ID No. 184, Seq. ID No. 188, Seq. ID No. 190, Seq. ID No. 192, Seq. ID No. 196, Seq. ID No. 198, Seq. ID No. 204, Seq. ID No. 208, Seq. ID No. 216, Seq. ID No. 220, Seq. ID No. 224, Seq. ID No. 226, Seq. ID No. 228, Seq. ID No. 232, Seq. ID No. 234, Seq. ID No. 240, Seq. ID No. 242, Seq. ID No. 248, Seq. ID No. 254, Seq. ID No. 262, Seq. ID No. 266, Seq. ID No. 270, Seq. ID No. 274, Seq. ID No. 276, Seq. ID No. 278, Seq. ID No. 284, Seq. ID No. 292, Seq. ID No. 294, Seq. ID No. 298, Seq. ID No. 302, Seq. ID No. 308, Seq. ID No. 310, Seq. ID No. 314, Seq. ID No. 318, Seq. ID No. 322, Seq. ID No. 326, Seq. ID No. 336, Seq. ID No. 344, Seq. ID No. 346, Seq. ID No. 354, Seq. ID No. 356, Seq. ID No. 362, Seq. ID No. 364, Seq. ID No. 368, Seq. ID No. 376, Seq. ID No. 380, Seq. ID No. 384, Seq. ID No. 390, Seq. ID No. 392, Seq. ID No. 398, Seq. ID No. 406, Seq. ID No. 412, Seq. ID No. 416, Seq. ID No. 418, Seq. ID No. 424, Seq. ID No. 426, Seq. ID No. 428, Seq. ID No. 430, Seq. ID No. 436, Seq. ID No. 438, Seq. ID No. 440, Seq. ID No. 442, Seq. ID No. 446, Seq. ID No. 448, Seq. ID No. 454, Seq. ID No. 462, Seq. ID No. 464, Seq. ID No. 472, Seq. ID No. 486, Seq. ID No. 502, Seq. ID No. 506, Seq. ID No. 512, Seq. ID No. 516, Seq. ID No. 518, Seq. ID No. 522, Seq. ID No. 524, Seq. ID No. 532, Seq. ID No. 534, Seq. ID No. 540, Seq. ID No. 542, Seq. ID No. 544, Seq. ID No. 552, Seq. ID No. 556, Seq. ID No. 562, Seq. ID No. 566, Seq. ID No. 570, Seq. ID No. 576, Seq. ID No. 580, Seq. ID No. 588, Seq. ID No. 594, Seq. ID No. 598, Seq. ID No. 604, Seq. ID No. 606, Seq. ID No. 610, Seq. ID No. 612, Seq. ID No. 614, Seq. ID No. 616, Seq. ID No. 624, Seq. ID No. 628, Seq. ID No. 630, Seq. ID No. 632, Seq. ID No. 636, Seq. ID No. 638, Seq. ID No. 648, Seq. ID No. 650, Seq. ID No. 662, Seq. ID No. 670, Seq. ID No. 674, Seq. ID No. 676, Seq. ID No. 682, Seq. ID No. 684, Seq. ID No. 690, Seq. ID No. 702, Seq. ID No. 704, Seq. ID No. 710, Seq. ID No. 714, Seq. ID No. 730, Seq. ID No. 734, Seq. ID No. 736, Seq. ID No. 738, Seq. ID No. 742, Seq. ID No. 748, Seq. ID No. 752, Seq. ID No. 754, Seq. ID No. 760, Seq. ID No. 766, Seq. ID No. 768, Seq. ID No. 772, Seq. ID No. 782, Seq. ID No. 784, Seq. ID No. 788, Seq. ID No. 792, Seq. ID No. 796, Seq. ID No. 800, Seq. ID No. 808, Seq. ID No. 818, Seq. ID No. 820, Seq. ID No. 824, Seq. ID No. 828, Seq. ID No. 832, Seq. ID No. 836, Seq. ID No. 838, Seq. ID No. 840, Seq. ID No. 848, Seq. ID No. 852, Seq. ID No. 856, Seq. ID No. 860, Seq. ID No. 868, Seq. ID No. 872, Seq. ID No. 876, Seq. ID No. 882, Seq. ID No. 886, Seq. ID No. 890, Seq. ID No. 892, Seq. ID No. 896, Seq. ID No. 902, Seq. ID No. 908, Seq. ID No. 916, Seq. ID No. 920, Seq. ID No. 926, Seq. ID No. 930, Seq. ID No. 936, Seq. ID No. 940, Seq. ID No. 950, Seq. ID No. 952, Seq. ID No. 956, Seq. ID No. 962, Seq. ID No. 966, Seq. ID No. 968, Seq. ID No. 972, Seq. ID No. 986, Seq. ID No. 994, Seq. ID No. 998, Seq. ID No. 1004, Seq. ID No. 1006, Seq. ID No. 1010, Seq. ID No. 1018, Seq. ID No. 1024, Seq. ID No. 1040, Seq. ID No. 1042, Seq. ID No. 1044, Seq. ID No. 1052, Seq. ID No. 1054, Seq. ID No. 1058, Seq. ID No. 1062, Seq. ID No. 1066, Seq. ID No. 1068, Seq. ID No. 1070, Seq. ID No. 1074, Seq. ID No. 1076, Seq. ID No. 1084, Seq. ID No. 1088, Seq. ID No. 1092, Seq. ID No. 1094, Seq. ID No. 1098, Seq. ID No. 1100, Seq. ID No. 1104, Seq. ID No. 1110, Seq. ID No. 1116, Seq. ID No. 1118, Seq. ID No. 1124, Seq. ID No. 1130, Seq. ID No. 1134, Seq. ID No. 1138, Seq. ID No. 1144, Seq. ID No. 1148, Seq. ID No. 1150, Seq. ID No. 1156, Seq. ID No. 1158, Seq. ID No. 1160, Seq. ID No. 1164, Seq. ID No. 1172, Seq. ID No. 1180, Seq. ID No. 1182, Seq. ID No. 1188, Seq. ID No. 1194, Seq. ID No. 1200, Seq. ID No. 1206, Seq. ID No. 1212, Seq. ID No. 1218, Seq. ID No. 1222, Seq. ID No. 1224, Seq. ID No. 1226, Seq. ID No. 1232, Seq. ID No. 1234, Seq. ID No. 1238, Seq. ID No. 1242, Seq. ID No. 1254, Seq. ID No. 1258, Seq. ID No. 1264, Seq. ID No. 1272, Seq. ID No. 1282, Seq. ID No. 1318, Seq. ID No. 1324, Seq. ID No. 1332, Seq. ID No. 1336, Seq. ID No. 1340, Seq. ID No. 1346, Seq. ID No. 1348, Seq. ID No. 1362, Seq. ID No. 1370, Seq. ID No. 1378, Seq. ID No. 1380, Seq. ID No. 1388, Seq. ID No. 1394, Seq. ID No. 1396, Seq. ID No. 1400, Seq. ID No. 1404, Seq. ID No. 1406, Seq. ID No. 1412, Seq. ID No. 1414, Seq. ID No. 1416, Seq. ID No. 1420, Seq. ID No. 1426, Seq. ID No. 1428, Seq. ID No. 1430, Seq. ID No. 1432, Seq. ID No. 1434, Seq. ID No. 1438, Seq. ID No. 1444, Seq. ID No. 1450, Seq. ID No. 1452, Seq. ID No. 1456, Seq. ID No. 1458, Seq. ID No. 1460, Seq. ID No. 1464, Seq. ID No. 1466, Seq. ID No. 1470, Seq. ID No. 1472, Seq. ID No. 1474, Seq. ID No. 1476, Seq. ID No. 1478, Seq. ID No. 1486, Seq. ID No. 1488, Seq. ID No. 1492, Seq. ID No. 1494, Seq. ID No. 1496, Seq. ID No. 1498, Seq. ID No. 1502, Seq. ID No. 1506, Seq. ID No. 1508, Seq. ID No. 1510, Seq. ID No. 1518, Seq. ID No. 1526, Seq. ID No. 1528, Seq. ID No. 1532, Seq. ID No. 1540, Seq. ID No. 1542, Seq. ID No. 1544, Seq. ID No. 1550, Seq. ID No. 1562, Seq. ID No. 1564, Seq. ID No. 1568, Seq. ID No. 1572, Seq. ID No. 1574, Seq. ID No. 1578, Seq. ID No. 1582, Seq. ID No. 1590, Seq. ID No. 1594, Seq. ID No. 1596, Seq. ID No. 1598, Seq. ID No. 1604, Seq. ID No. 1608, Seq. ID No. 1612, Seq. ID No. 1614, Seq. ID No. 1620, Seq. ID No. 1624, Seq. ID No. 1628, Seq. ID No. 1632, Seq. ID No. 1638, Seq. ID No. 1640, Seq. ID No. 1642, Seq. ID No. 1648, Seq. ID No. 1654, Seq. ID No. 1658, Seq. ID No. 1666, Seq. ID No. 1670, Seq. ID No. 1682, Seq. ID No. 1686, Seq. ID No. 1690, Seq. ID No. 1692, Seq. ID No. 1698, Seq. ID No. 1700, Seq. ID No. 1708, Seq. ID No. 1712, Seq. ID No. 1714, Seq. ID No. 1720, Seq. ID No. 1724, Seq. ID No. 1730, Seq. ID No. 1736, Seq. ID No. 1738, Seq. ID No. 1742, Seq. ID No. 1748, Seq. ID No. 1752, Seq. ID No. 1758, Seq. ID No. 1760, Seq. ID No. 1762, Seq. ID No. 1772, Seq. ID No. 1778, Seq. ID No. 1786, Seq. ID No. 1790, Seq. ID No. 1792, Seq. ID No. 1798, Seq. ID No. 1802, Seq. ID No. 1804, Seq. ID No. 1806, Seq. ID No. 1812, Seq. ID No. 1818, Seq. ID No. 1820, Seq. ID No. 1822, Seq. ID No. 1826, Seq. ID No. 1830, Seq. ID No. 1838, Seq. ID No. 1844, Seq. ID No. 1846, Seq. ID No. 1848, Seq. ID No. 1850, Seq. ID No. 1852, Seq. ID No. 1856, Seq. ID No. 1860, Seq. ID No. 1864, Seq. ID No. 1866, Seq. ID No. 1868, Seq. ID No. 1872, Seq. ID No. 1874, Seq. ID No. 1878, Seq. ID No. 1886, Seq. ID No. 1888, Seq. ID No. 1890, Seq. ID No. 1896, Seq. ID No. 1898, Seq. ID No. 1900, Seq. ID No. 1904, Seq. ID No. 1906, Seq. ID No. 1914, Seq. ID No. 1920, Seq. ID No. 1924, Seq. ID No. 1926, Seq. ID No. 1932, Seq. ID No. 1936, Seq. ID No. 1938, Seq. ID No. 1944, Seq. ID No. 1946, Seq. ID No. 1950, Seq. ID No. 1954, Seq. ID No. 1958, Seq. ID No. 1960, Seq. ID No. 1962, Seq. ID No. 1966, Seq. ID No. 1968, Seq. ID No. 1972, Seq. ID No. 1976, Seq. ID No. 1980, Seq. ID No. 1984, Seq. ID No. 1988, Seq. ID No. 1990, Seq. ID No. 1992, Seq. ID No. 2000, Seq. ID No. 2002, Seq. ID No. 2004, Seq. ID No. 2010, Seq. ID No. 2016, Seq. ID No. 2020, Seq. ID No. 2022, Seq. ID No. 2032, Seq. ID No. 2036, Seq. ID No. 2038, Seq. ID No. 2046, Seq. ID No. 2048, Seq. ID No. 2056, Seq. ID No. 2060, Seq. ID No. 2066, Seq. ID No. 2068, Seq. ID No. 2070, Seq. ID No. 2072, Seq. ID No. 2074, Seq. ID No. 2080, Seq. ID No. 2092, Seq. ID No. 2094, Seq. ID No. 2098, Seq. ID No. 2100, Seq. ID No. 2104, Seq. ID No. 2116, Seq. ID No. 2120, Seq. ID No. 2128, Seq. ID No. 2136, Seq. ID No. 2138, Seq. ID No. 2142, Seq. ID No. 2144, Seq. ID No. 2148, Seq. ID No. 2154, Seq. ID No. 2160, Seq. ID No. 2162, Seq. ID No. 2166, Seq. ID No. 2170, Seq. ID No. 2180, Seq. ID No. 2184, Seq. ID No. 2186, Seq. ID No. 2190, Seq. ID No. 2192, Seq. ID No. 2194, Seq. ID No. 2196, Seq. ID No. 2200, Seq. ID No. 2208, Seq. ID No. 2218, Seq. ID No. 2220, Seq. ID No. 2222, Seq. ID No. 2224, Seq. ID No. 2228, Seq. ID No. 2230, Seq. ID No. 2232, Seq. ID No. 2234, Seq. ID No. 2242, Seq. ID No. 2246, Seq. ID No. 2252, Seq. ID No. 2254, Seq. ID No. 2258, Seq. ID No. 2260, Seq. ID No. 2264, Seq. ID No. 2268, Seq. ID No. 2270, Seq. ID No. 2276, Seq. ID No. 2282, Seq. ID No. 2288, Seq. ID No. 2298, Seq. ID No. 2300, Seq. ID No. 2302, Seq. ID No. 2316, Seq. ID No. 2330, Seq. ID No. 2334, Seq. ID No. 2336, Seq. ID No. 2340, Seq. ID No. 2344, Seq. ID No. 2346, Seq. ID No. 2350, Seq. ID No. 2352, Seq. ID No. 2358, Seq. ID No. 2360, Seq. ID No. 2364, Seq. ID No. 2366, Seq. ID No. 2370, Seq. ID No. 2374, Seq. ID No. 2382, Seq. ID No. 2392, Seq. ID No. 2400, Seq. ID No. 2402, Seq. ID No. 2406, Seq. ID No. 2410, Seq. ID No. 2416, Seq. ID No. 2418, Seq. ID No. 2422, Seq. ID No. 2424, Seq. ID No. 2428, Seq. ID No. 2430, Seq. ID No. 2432, Seq. ID No. 2436, Seq. ID No. 2438, Seq. ID No. 2440, Seq. ID No. 2444, Seq. ID No. 2446, Seq. ID No. 2450, Seq. ID No. 2452, Seq. ID No. 2454, Seq. ID No. 2458, Seq. ID No. 2464, Seq. ID No. 2466, Seq. ID No. 2470, Seq. ID No. 2472, Seq. ID No. 2476, Seq. ID No. 2478, Seq. ID No. 2484, Seq. ID No. 2486, Seq. ID No. 2492, Seq. ID No. 2496, Seq. ID No. 2506, Seq. ID No. 2508, Seq. ID No. 2514, Seq. ID No. 2520, Seq. ID No. 2526, Seq. ID No. 2530, Seq. ID No. 2536, Seq. ID No. 2538, Seq. ID No. 2540, Seq. ID No. 2550, Seq. ID No. 2554, Seq. ID No. 2564, Seq. ID No. 2568, Seq. ID No. 2576, Seq. ID No. 2578, Seq. ID No. 2582, Seq. ID No. 2590, Seq. ID No. 2594, Seq. ID No. 2596, Seq. ID No. 2600, Seq. ID No. 2604, Seq. ID No. 2608, Seq. ID No. 2610, Seq. ID No. 2614, Seq. ID No. 2618, Seq. ID No. 2620, Seq. ID No. 2624, Seq. ID No. 2626, Seq. ID No. 2632, Seq. ID No. 2634, Seq. ID No. 2640, Seq. ID No. 2642, Seq. ID No. 2646, Seq. ID No. 2650, Seq. ID No. 2652, Seq. ID No. 2656, Seq. ID No. 2660, Seq. ID No. 2664, Seq. ID No. 2668, Seq. ID No. 2670, Seq. ID No. 2672, Seq. ID No. 2678, Seq. ID No. 2688, Seq. ID No. 2690, Seq. ID No. 2694, Seq. ID No. 2700, Seq. ID No. 2704, Seq. ID No. 2710, Seq. ID No. 2712, Seq. ID No. 2716, Seq. ID No. 2720, Seq. ID No. 2726, Seq. ID No. 2728, Seq. ID No. 2738, Seq. ID No. 2744, Seq. ID No. 2748, Seq. ID No. 2752, Seq. ID No. 2754, Seq. ID No. 2760, Seq. ID No. 2764, Seq. ID No. 2766, Seq. ID No. 2768, Seq. ID No. 2772, Seq. ID No. 2774, Seq. ID No. 2776, Seq. ID No. 2782, Seq. ID No. 2788, Seq. ID No. 2790, Seq. ID No. 2792, Seq. ID No. 2794, Seq. ID No. 2798, Seq. ID No. 2800, Seq. ID No. 2804, Seq. ID No. 2806, Seq. ID No. 2812, Seq. ID No. 2816, Seq. ID No. 2824, Seq. ID No. 2828, Seq. ID No. 2844, Seq. ID No. 2848, Seq. ID No. 2864, Seq. ID No. 2868, Seq. ID No. 2870, Seq. ID No. 2872, Seq. ID No. 2878, Seq. ID No. 2882, Seq. ID No. 2892, Seq. ID No. 2900, Seq. ID No. 2902, Seq. ID No. 2904, Seq. ID No. 2908, Seq. ID No. 2912, Seq. ID No. 2914, Seq. ID No. 2918, Seq. ID No. 2920, Seq. ID No. 2922, Seq. ID No. 2924, Seq. ID No. 2928, Seq. ID No. 2934, Seq. ID No. 2946, Seq. ID No. 2954, Seq. ID No. 2958, Seq. ID No. 2964, Seq. ID No. 2972, Seq. ID No. 2974, Seq. ID No. 2978, Seq. ID No. 2980, Seq. ID No. 2984, Seq. ID No. 2986, Seq. ID No. 2992, Seq. ID No. 2994, Seq. ID No. 3000, Seq. ID No. 3002, Seq. ID No. 3006, Seq. ID No. 3012, Seq. ID No. 3020, Seq. ID No. 3028, Seq. ID No. 3034, Seq. ID No. 3036, Seq. ID No. 3040, Seq. ID No. 3044, Seq. ID No. 3046, Seq. ID No. 3050, Seq. ID No. 3056, Seq. ID No. 3060, Seq. ID No. 3062, Seq. ID No. 3068, Seq. ID No. 3070, Seq. ID No. 3082, Seq. ID No. 3090, Seq. ID No. 3092, Seq. ID No. 3094, Seq. ID No. 3098, Seq. ID No. 3104, Seq. ID No. 3108, Seq. ID No. 3112, Seq. ID No. 3118, Seq. ID No. 3126, Seq. ID No. 3134, Seq. ID No. 3136, Seq. ID No. 3138, Seq. ID No. 3142, Seq. ID No. 3144, Seq. ID No. 3150, Seq. ID No. 3156, Seq. ID No. 3158, Seq. ID No. 3160, Seq. ID No. 3162, Seq. ID No. 3166, Seq. ID No. 3172, Seq. ID No. 3178, Seq. ID No. 3180, Seq. ID No. 3184, Seq. ID No. 3186, Seq. ID No. 3190, Seq. ID No. 3192, Seq. ID No. 3198, Seq. ID No. 3204, Seq. ID No. 3206, Seq. ID No. 3212, Seq. ID No. 3218, Seq. ID No. 3222, Seq. ID No. 3228, Seq. ID No. 3230, Seq. ID No. 3234, Seq. ID No. 3236, Seq. ID No. 3240, Seq. ID No. 3250, Seq. ID No. 3252, Seq. ID No. 3254, Seq. ID No. 3258, Seq. ID No. 3260, Seq. ID No. 3264, Seq. ID No. 3266, Seq. ID No. 3272, Seq. ID No. 3274, Seq. ID No. 3278, Seq. ID No. 3284, Seq. ID No. 3286, Seq. ID No. 3288, Seq. ID No. 3290, Seq. ID No. 3294, Seq. ID No. 3296, Seq. ID No. 3300, Seq. ID No. 3304, Seq. ID No. 3308, Seq. ID No. 3314, Seq. ID No. 3316, Seq. ID No. 3320, Seq. ID No. 3324, Seq. ID No. 3330, Seq. ID No. 3336, Seq. ID No. 3338, Seq. ID No. 3348, Seq. ID No. 3356, Seq. ID No. 3360, Seq. ID No. 3364, Seq. ID No. 3368, Seq. ID No. 3378, Seq. ID No. 3380, Seq. ID No. 3384, Seq. ID No. 3388, Seq. ID No. 3400, Seq. ID No. 3402, Seq. ID No. 3404, Seq. ID No. 3410, Seq. ID No. 3414, Seq. ID No. 3416, Seq. ID No. 3418, Seq. ID No. 3426, Seq. ID No. 3428, Seq. ID No. 3432, Seq. ID No. 3436, Seq. ID No. 3438, Seq. ID No. 3440, Seq. ID No. 3448, Seq. ID No. 3460, Seq. ID No. 3462, Seq. ID No. 3468, Seq. ID No. 3470, Seq. ID No. 3472, Seq. ID No. 3474, Seq. ID No. 3476, Seq. ID No. 3480, Seq. ID No. 3484, Seq. ID No. 3486, Seq. ID No. 3488, Seq. ID No. 3496, Seq. ID No. 3502, Seq. ID No. 3506, Seq. ID No. 3508, Seq. ID No. 3510, Seq. ID No. 3512, Seq. ID No. 3520, Seq. ID No. 3522, Seq. ID No. 3526, Seq. ID No. 3530, Seq. ID No. 3534, Seq. ID No. 3538, Seq. ID No. 3544, Seq. ID No. 3550, Seq. ID No. 3554, Seq. ID No. 3562, Seq. ID No. 3566, Seq. ID No. 3570, Seq. ID No. 3576, Seq. ID No. 3580, Seq. ID No. 3584, Seq. ID No. 3586, Seq. ID No. 3590, Seq. ID No. 3600, Seq. ID No. 3606, Seq. ID No. 3608, Seq. ID No. 3610, Seq. ID No. 3612, Seq. ID No. 3616, Seq. ID No. 3620, Seq. ID No. 3630, Seq. ID No. 3636, Seq. ID No. 3640, Seq. ID No. 3642, Seq. ID No. 3644, Seq. ID No. 3646, Seq. ID No. 3652, Seq. ID No. 3654, Seq. ID No. 3660, Seq. ID No. 3664, Seq. ID No. 3666, Seq. ID No. 3668, Seq. ID No. 3670, Seq. ID No. 3676, Seq. ID No. 3682, Seq. ID No. 3690, Seq. ID No. 3696, Seq. ID No. 3700, Seq. ID No. 3710, Seq. ID No. 3716, Seq. ID No. 3722, Seq. ID No. 3728, Seq. ID No. 3730, Seq. ID No. 3734, Seq. ID No. 3736, Seq. ID No. 3740, Seq. ID No. 3742, Seq. ID No. 3748, Seq. ID No. 3752, Seq. ID No. 3760, Seq. ID No. 3772, Seq. ID No. 3776, Seq. ID No. 3780, Seq. ID No. 3784, Seq. ID No. 3792, Seq. ID No. 3798, Seq. ID No. 3804, Seq. ID No. 3806, Seq. ID No. 3812, Seq. ID No. 3814, Seq. ID No. 3820, Seq. ID No. 3824, Seq. ID No. 3826, Seq. ID No. 3834, Seq. ID No. 3836, Seq. ID No. 3844, Seq. ID No. 3848, Seq. ID No. 3856, Seq. ID No. 3858, Seq. ID No. 3864, Seq. ID No. 3868, Seq. ID No. 3874, Seq. ID No. 3878, Seq. ID No. 3882, Seq. ID No. 3884, Seq. ID No. 3886, Seq. ID No. 3894, Seq. ID No. 3898, Seq. ID No. 3902, Seq. ID No. 3908, Seq. ID No. 3914, Seq. ID No. 3916, Seq. ID No. 3918, Seq. ID No. 3920, Seq. ID No. 3926, Seq. ID No. 3930, Seq. ID No. 3934, Seq. ID No. 3936, Seq. ID No. 3938, Seq. ID No. 3942, Seq. ID No. 3944, Seq. ID No. 3946, Seq. ID No. 3950, Seq. ID No. 3952, Seq. ID No. 3956, Seq. ID No. 3958, Seq. ID No. 3972, Seq. ID No. 3976, Seq. ID No. 3984, Seq. ID No. 3986, Seq. ID No. 3988, Seq. ID No. 3992, Seq. ID No. 4004, Seq. ID No. 4010, Seq. ID No. 4014, Seq. ID No. 4016, Seq. ID No. 4018, Seq. ID No. 4022, Seq. ID No. 4024, Seq. ID No. 4030, Seq. ID No. 4032, Seq. ID No. 4036, Seq. ID No. 4038, Seq. ID No. 4040, Seq. ID No. 4050, Seq. ID No. 4056, Seq. ID No. 4058, Seq. ID No. 4062, Seq. ID No. 4064, Seq. ID No. 4068, Seq. ID No. 4070, Seq. ID No. 4076, Seq. ID No. 4078, Seq. ID No. 4080, Seq. ID No. 4088, Seq. ID No. 4090, Seq. ID No. 4096, Seq. ID No. 4098, Seq. ID No. 4100, Seq. ID No. 4106, Seq. ID No. 4112, Seq. ID No. 4118, Seq. ID No. 4122, Seq. ID No. 4124, Seq. ID No. 4128, Seq. ID No. 4130, Seq. ID No. 4134, Seq. ID No. 4138, Seq. ID No. 4146, Seq. ID No. 4150, Seq. ID No. 4158, Seq. ID No. 4162, Seq. ID No. 4164, Seq. ID No. 4168, Seq. ID No. 4174, Seq. ID No. 4180, Seq. ID No. 4186, Seq. ID No. 4192, Seq. ID No. 4194, Seq. ID No. 4196, Seq. ID No. 4198, Seq. ID No. 4200, Seq. ID No. 4202, Seq. ID No. 4204, Seq. ID No. 4206, Seq. ID No. 4212, Seq. ID No. 4216, Seq. ID No. 4218, Seq. ID No. 4220, Seq. ID No. 4236, Seq. ID No. 4238, Seq. ID No. 4240, Seq. ID No. 4244, Seq. ID No. 4252, Seq. ID No. 4256, Seq. ID No. 4264, Seq. ID No. 4268, Seq. ID No. 4272, Seq. ID No. 4274, Seq. ID No. 4280, Seq. ID No. 4282, Seq. ID No. 4284, Seq. ID No. 4292, Seq. ID No. 4296, Seq. ID No. 4300, Seq. ID No. 4302, Seq. ID No. 4304, Seq. ID No. 4308, Seq. ID No. 4310, Seq. ID No. 4314, Seq. ID No. 4318, Seq. ID No. 4324, Seq. ID No. 4326, Seq. ID No. 4330, Seq. ID No. 4332, Seq. ID No. 4334, Seq. ID No. 4340, Seq. ID No. 4344, Seq. ID No. 4348, Seq. ID No. 4354, Seq. ID No. 4356, Seq. ID No. 4358, Seq. ID No. 4366, Seq. ID No. 4370, Seq. ID No. 4374, Seq. ID No. 4376, Seq. ID No. 4378, Seq. ID No. 4380, Seq. ID No. 4382, Seq. ID No. 4384, Seq. ID No. 4392, Seq. ID No. 4394, Seq. ID No. 4398, Seq. ID No. 4402, Seq. ID No. 4412, Seq. ID No. 4418, Seq. ID No. 4424, Seq. ID No. 4432, Seq. ID No. 4434, Seq. ID No. 4436, Seq. ID No. 4444, Seq. ID No. 4448, Seq. ID No. 4452, Seq. ID No. 4460, Seq. ID No. 4462, Seq. ID No. 4468, Seq. ID No. 4472, Seq. ID No. 4476, Seq. ID No. 4480, Seq. ID No. 4482, Seq. ID No. 4488, Seq. ID No. 4492, Seq. ID No. 4502, Seq. ID No. 4506, Seq. ID No. 4512, Seq. ID No. 4524, Seq. ID No. 4532, Seq. ID No. 4534, Seq. ID No. 4538, Seq. ID No. 4540, Seq. ID No. 4542, Seq. ID No. 4544, Seq. ID No. 4548, Seq. ID No. 4550, Seq. ID No. 4560, Seq. ID No. 4566, Seq. ID No. 4568, Seq. ID No. 4572, Seq. ID No. 4576, Seq. ID No. 4578, Seq. ID No. 4582, Seq. ID No. 4586, Seq. ID No. 4592, Seq. ID No. 4596, Seq. ID No. 4598, Seq. ID No. 4604, Seq. ID No. 4608, Seq. ID No. 4610, Seq. ID No. 4612, Seq. ID No. 4616, Seq. ID No. 4624, Seq. ID No. 4628, Seq. ID No. 4632, Seq. ID No. 4636, Seq. ID No. 4638, Seq. ID No. 4644, Seq. ID No. 4650, Seq. ID No. 4652, Seq. ID No. 4654, Seq. ID No. 4658, Seq. ID No. 4662, Seq. ID No. 4664, Seq. ID No. 4670, Seq. ID No. 4672, Seq. ID No. 4674, Seq. ID No. 4680, Seq. ID No. 4686, Seq. ID No. 4694, Seq. ID No. 4698, Seq. ID No. 4704, Seq. ID No. 4706, Seq. ID No. 4714, Seq. ID No. 4718, Seq. ID No. 4720, Seq. ID No. 4726, Seq. ID No. 4730, Seq. ID No. 4732, Seq. ID No. 4734, Seq. ID No. 4740, Seq. ID No. 4744, Seq. ID No. 4746, Seq. ID No. 4748, Seq. ID No. 4750, Seq. ID No. 4752, Seq. ID No. 4758, Seq. ID No. 4766, Seq. ID No. 4770, Seq. ID No. 4778, Seq. ID No. 4780, Seq. ID No. 4782, Seq. ID No. 4788, Seq. ID No. 4790, Seq. ID No. 4792, Seq. ID No. 4798, Seq. ID No. 4804, Seq. ID No. 4808, Seq. ID No. 4810, Seq. ID No. 4812, Seq. ID No. 4814, Seq. ID No. 4816, Seq. ID No. 4822, Seq. ID No. 4824, Seq. ID No. 4826, Seq. ID No. 4828, Seq. ID No. 4834, Seq. ID No. 4836, Seq. ID No. 4838, Seq. ID No. 4844, Seq. ID No. 4846, Seq. ID No. 4848, Seq. ID No. 4850, Seq. ID No. 4860, Seq. ID No. 4862, Seq. ID No. 4866, Seq. ID No. 4868, Seq. ID No. 4876, Seq. ID No. 4878, Seq. ID No. 4880, Seq. ID No. 4882, Seq. ID No. 4884, Seq. ID No. 4886, Seq. ID No. 4888, Seq. ID No. 4894, Seq. ID No. 4900, Seq. ID No. 4902, Seq. ID No. 4904, Seq. ID No. 4906, Seq. ID No. 4908, Seq. ID No. 4912, Seq. ID No. 4916, Seq. ID No. 4918, Seq. ID No. 4920, Seq. ID No. 4922, Seq. ID No. 4928, Seq. ID No. 4932, Seq. ID No. 4936, Seq. ID No. 4940, Seq. ID No. 4942, Seq. ID No. 4944, Seq. ID No. 4946, Seq. ID No. 4948, Seq. ID No. 4954, Seq. ID No. 4958, Seq. ID No. 4960, Seq. ID No. 4964, Seq. ID No. 4966, Seq. ID No. 4970, Seq. ID No. 4972, Seq. ID No. 4974, Seq. ID No. 4980, Seq. ID No. 4986, Seq. ID No. 4990, Seq. ID No. 4992, Seq. ID No. 4996, Seq. ID No. 4998, Seq. ID No. 5002, Seq. ID No. 5008, Seq. ID No. 5010, Seq. ID No. 5018, Seq. ID No. 5022, Seq. ID No. 5028, Seq. ID No. 5030, Seq. ID No. 5040, Seq. ID No. 5048, Seq. ID No. 5052, Seq. ID No. 5054, Seq. ID No. 5056, Seq. ID No. 5060, Seq. ID No. 5062, Seq. ID No. 5070, Seq. ID No. 5078, Seq. ID No. 5080, Seq. ID No. 5082, Seq. ID No. 5084, Seq. ID No. 5088, Seq. ID No. 5092, Seq. ID No. 5094, Seq. ID No. 5098, Seq. ID No. 5104, Seq. ID No. 5106, Seq. ID No. 5110, Seq. ID No. 5112, Seq. ID No. 5114, Seq. ID No. 5116, Seq. ID No. 5118, Seq. ID No. 5128, Seq. ID No. 5142, Seq. ID No. 5146, Seq. ID No. 5150, Seq. ID No. 5154, Seq. ID No. 5158, Seq. ID No. 5166, Seq. ID No. 5170, Seq. ID No. 5172, Seq. ID No. 5178, Seq. ID No. 5184, Seq. ID No. 5186, Seq. ID No. 5188, Seq. ID No. 5194, Seq. ID No. 5196, Seq. ID No. 5202, Seq. ID No. 5204, Seq. ID No. 5206, Seq. ID No. 5210, Seq. ID No. 5212, Seq. ID No. 5216, Seq. ID No. 5220, Seq. ID No. 5228, Seq. ID No. 5232, Seq. ID No. 5236, Seq. ID No. 5238, Seq. ID No. 5240, Seq. ID No. 5244, Seq. ID No. 5246, Seq. ID No. 5248, Seq. ID No. 5254, Seq. ID No. 5256, Seq. ID No. 5258, Seq. ID No. 5262, Seq. ID No. 5264, Seq. ID No. 5272, Seq. ID No. 5276, Seq. ID No. 5284, Seq. ID No. 5292, Seq. ID No. 5300, Seq. ID No. 5306, Seq. ID No. 5310, Seq. ID No. 5312, Seq. ID No. 5314, Seq. ID No. 5320, Seq. ID No. 5324, Seq. ID No. 5326, Seq. ID No. 5336, Seq. ID No. 5338, Seq. ID No. 5342, Seq. ID No. 5346, Seq. ID No. 5352, Seq. ID No. 5356, Seq. ID No. 5364, Seq. ID No. 5366, Seq. ID No. 5368, Seq. ID No. 5372, Seq. ID No. 5374, Seq. ID No. 5382, Seq. ID No. 5384, Seq. ID No. 5388, Seq. ID No. 5390, Seq. ID No. 5392, Seq. ID No. 5394, Seq. ID No. 5396, Seq. ID No. 5398, Seq. ID No. 5400, Seq. ID No. 5402, Seq. ID No. 5404, Seq. ID No. 5410, Seq. ID No. 5412, Seq. ID No. 5416, Seq. ID No. 5418, Seq. ID No. 5420, Seq. ID No. 5422, Seq. ID No. 5424, Seq. ID No. 5428, Seq. ID No. 5432, Seq. ID No. 5434, Seq. ID No. 5440, Seq. ID No. 5442, Seq. ID No. 5444, Seq. ID No. 5448, Seq. ID No. 5450, Seq. ID No. 5452, Seq. ID No. 5454, Seq. ID No. 5456, Seq. ID No. 5458, Seq. ID No. 5474, Seq. ID No. 5478, Seq. ID No. 5484, Seq. ID No. 5488, Seq. ID No. 5492, Seq. ID No. 5496, Seq. ID No. 5500, Seq. ID No. 5504, Seq. ID No. 5508, Seq. ID No. 5510, Seq. ID No. 5514, Seq. ID No. 5528, Seq. ID No. 5532, Seq. ID No. 5538, Seq. ID No. 5540, Seq. ID No. 5542, Seq. ID No. 5548, Seq. ID No. 5554, Seq. ID No. 5558, Seq. ID No. 5560, Seq. ID No. 5564, Seq. ID No. 5568, Seq. ID No. 5572, Seq. ID No. 5576, Seq. ID No. 5582, Seq. ID No. 5586, Seq. ID No. 5592, Seq. ID No. 5600, Seq. ID No. 5602, Seq. ID No. 5606, Seq. ID No. 5616, Seq. ID No. 5622, Seq. ID No. 5630, Seq. ID No. 5632, Seq. ID No. 5638, Seq. ID No. 5648, Seq. ID No. 5652, Seq. ID No. 5656, Seq. ID No. 5658, Seq. ID No. 5662, Seq. ID No. 5666, Seq. ID No. 5670, Seq. ID No. 5672, Seq. ID No. 5674, Seq. ID No. 5680, Seq. ID No. 5686, Seq. ID No. 5694, Seq. ID No. 5698, Seq. ID No. 5700, Seq. ID No. 5702, Seq. ID No. 5708, Seq. ID No. 5716, Seq. ID No. 5718, Seq. ID No. 5724, Seq. ID No. 5728, Seq. ID No. 5736, Seq. ID No. 5740, Seq. ID No. 5750, Seq. ID No. 5752, Seq. ID No. 5756, Seq. ID No. 5762, Seq. ID No. 5772, Seq. ID No. 5776, Seq. ID No. 5778, Seq. ID No. 5780, Seq. ID No. 5784, Seq. ID No. 5790, Seq. ID No. 5796, Seq. ID No. 5802, Seq. ID No. 5804, Seq. ID No. 5810, Seq. ID No. 5816, Seq. ID No. 5820, Seq. ID No. 5826, Seq. ID No. 5834, Seq. ID No. 5836, Seq. ID No. 5842, Seq. ID No. 5846, Seq. ID No. 5848, Seq. ID No. 5850, Seq. ID No. 5852, Seq. ID No. 5854, Seq. ID No. 5856, Seq. ID No. 5860, Seq. ID No. 5866, Seq. ID No. 5872, Seq. ID No. 5876, Seq. ID No. 5878, Seq. ID No. 5882, Seq. ID No. 5888, Seq. ID No. 5892, Seq. ID No. 5896, Seq. ID No. 5898, Seq. ID No. 5902, Seq. ID No. 5904, Seq. ID No. 5912, Seq. ID No. 5914, Seq. ID No. 5916, Seq. ID No. 5918, Seq. ID No. 5920, Seq. ID No. 5930, Seq. ID No. 5932, Seq. ID No. 5934, Seq. ID No. 5938, Seq. ID No. 5942, Seq. ID No. 5944, Seq. ID No. 5948, Seq. ID No. 5952, Seq. ID No. 5954, Seq. ID No. 5956, Seq. ID No. 5958, Seq. ID No. 5962, Seq. ID No. 5970, Seq. ID No. 5974, Seq. ID No. 5980, Seq. ID No. 5982, Seq. ID No. 5986, Seq. ID No. 5990, Seq. ID No. 5994, Seq. ID No. 6002, Seq. ID No. 6004, Seq. ID No. 6012, Seq. ID No. 6016, Seq. ID No. 6018, Seq. ID No. 6022, Seq. ID No. 6028, Seq. ID No. 6030, Seq. ID No. 6034, Seq. ID No. 6036, Seq. ID No. 6042, Seq. ID No. 6044, Seq. ID No. 6054, Seq. ID No. 6058, Seq. ID No. 6066, Seq. ID No. 6072, Seq. ID No. 6080, Seq. ID No. 6086, Seq. ID No. 6090, Seq. ID No. 6096, Seq. ID No. 6098, Seq. ID No. 6102, Seq. ID No. 6104, Seq. ID No. 6112, Seq. ID No. 6114, Seq. ID No. 6118, Seq. ID No. 6124, Seq. ID No. 6126, Seq. ID No. 6130, Seq. ID No. 6132, Seq. ID No. 6134, Seq. ID No. 6142, Seq. ID No. 6146, Seq. ID No. 6150, Seq. ID No. 6158, Seq. ID No. 6166, Seq. ID No. 6170, Seq. ID No. 6172, Seq. ID No. 6176, Seq. ID No. 6178, Seq. ID No. 6182, Seq. ID No. 6188, Seq. ID No. 6196, Seq. ID No. 6202, Seq. ID No. 6210, Seq. ID No. 6214, Seq. ID No. 6216, Seq. ID No. 6220, Seq. ID No. 6234, Seq. ID No. 6236, Seq. ID No. 6244, Seq. ID No. 6250, Seq. ID No. 6252, Seq. ID No. 6258, Seq. ID No. 6262, Seq. ID No. 6264, Seq. ID No. 6272, Seq. ID No. 6276, Seq. ID No. 6278, Seq. ID No. 6282, Seq. ID No. 6288, Seq. ID No. 6298, Seq. ID No. 6300, Seq. ID No. 6306, Seq. ID No. 6312, Seq. ID No. 6316, Seq. ID No. 6322, Seq. ID No. 6324, Seq. ID No. 6328, Seq. ID No. 6332, Seq. ID No. 6334, Seq. ID No. 6338, Seq. ID No. 6340, Seq. ID No. 6342, Seq. ID No. 6344, Seq. ID No. 6346, Seq. ID No. 6354, Seq. ID No. 6358, Seq. ID No. 6360, Seq. ID No. 6362, Seq. ID No. 6368, Seq. ID No. 6370, Seq. ID No. 6374, Seq. ID No. 6376, Seq. ID No. 6378, Seq. ID No. 6380, Seq. ID No. 6390, Seq. ID No. 6396, Seq. ID No. 6400, Seq. ID No. 6404, Seq. ID No. 6408, Seq. ID No. 6412, Seq. ID No. 6414, Seq. ID No. 6418, Seq. ID No. 6424, Seq. ID No. 6434, Seq. ID No. 6438, Seq. ID No. 6440, Seq. ID No. 6446, Seq. ID No. 6450, Seq. ID No. 6452, Seq. ID No. 6458, Seq. ID No. 6460, Seq. ID No. 6462, Seq. ID No. 6464, Seq. ID No. 6478, Seq. ID No. 6482, Seq. ID No. 6484, Seq. ID No. 6488, Seq. ID No. 6490, Seq. ID No. 6492, Seq. ID No. 6494, Seq. ID No. 6496, Seq. ID No. 6504, Seq. ID No. 6506, Seq. ID No. 6512, Seq. ID No. 6516, Seq. ID No. 6520, Seq. ID No. 6524, Seq. ID No. 6526, Seq. ID No. 6530, Seq. ID No. 6532, Seq. ID No. 6534, Seq. ID No. 6538, Seq. ID No. 6546, Seq. ID No. 6552, Seq. ID No. 6554, Seq. ID No. 6560, Seq. ID No. 6564, Seq. ID No. 6568, Seq. ID No. 6578, Seq. ID No. 6580, Seq. ID No. 6584, Seq. ID No. 6586, Seq. ID No. 6588, Seq. ID No. 6590, Seq. ID No. 6604, Seq. ID No. 6606, Seq. ID No. 6610, Seq. ID No. 6614, Seq. ID No. 6616, Seq. ID No. 6618, Seq. ID No. 6622, Seq. ID No. 6624, Seq. ID No. 6628, Seq. ID No. 6630, Seq. ID No. 6634, and Seq. ID No. 6640.

53. The Alloiococcus otitidis polypeptide of claim 36 wherein the polypeptide is identified as an Alloiococcus otitidis polypeptide by GeneMark™ ORF finder program, and is selected from one of Seq. ID No. 2, Seq. ID No. 4, Seq. ID No. 12, Seq. ID No. 14, Seq. ID No. 16, Seq. ID No. 18, Seq. ID No. 26, Seq. ID No. 28, Seq. ID No. 30, Seq. ID No. 36, Seq. ID No. 38, Seq. ID No. 40, Seq. ID No. 42, Seq. ID No. 44, Seq. ID No. 46, Seq. ID No. 50, Seq. ID No. 54, Seq. ID No. 58, Seq. ID No. 62, Seq. ID No. 70, Seq. ID No. 72, Seq. ID No. 76, Seq. ID No. 82, Seq. ID No. 84, Seq. ID No. 90, Seq. ID No. 96, Seq. ID No. 102, Seq. ID No. 112, Seq. ID No. 120, Seq. ID No. 126, Seq. ID No. 130, Seq. ID No. 136, Seq. ID No. 138, Seq. ID No. 142, Seq. ID No. 148, Seq. ID No. 152, Seq. ID No. 156, Seq. ID No. 168, Seq. ID No. 172, Seq. ID No. 182, Seq. ID No. 186, Seq. ID No. 188, Seq. ID No. 190, Seq. ID No. 192, Seq. ID No. 196, Seq. ID No. 200, Seq. ID No. 208, Seq. ID No. 214, Seq. ID No. 220, Seq. ID No. 222, Seq. ID No. 226, Seq. ID No. 228, Seq. ID No. 230, Seq. ID No. 234, Seq. ID No. 240, Seq. ID No. 242, Seq. ID No. 244, Seq. ID No. 254, Seq. ID No. 262, Seq. ID No. 264, Seq. ID No. 268, Seq. ID No. 274, Seq. ID No. 276, Seq. ID No. 278, Seq. ID No. 280, Seq. ID No. 292, Seq. ID No. 294, Seq. ID No. 296, Seq. ID No. 300, Seq. ID No. 304, Seq. ID No. 310, Seq. ID No. 312, Seq. ID No. 316, Seq. ID No. 320, Seq. ID No. 324, Seq. ID No. 332, Seq. ID No. 342, Seq. ID No. 346, Seq. ID No. 352, Seq. ID No. 356, Seq. ID No. 362, Seq. ID No. 364, Seq. ID No. 366, Seq. ID No. 376, Seq. ID No. 380, Seq. ID No. 382, Seq. ID No. 384, Seq. ID No. 390, Seq. ID No. 392, Seq. ID No. 396, Seq. ID No. 400, Seq. ID No. 406, Seq. ID No. 410, Seq. ID No. 414, Seq. ID No. 418, Seq. ID No. 422, Seq. ID No. 424, Seq. ID No. 426, Seq. ID No. 428, Seq. ID No. 430, Seq. ID No. 436, Seq. ID No. 438, Seq. ID No. 440, Seq. ID No. 442, Seq. ID No. 444, Seq. ID No. 446, Seq. ID No. 448, Seq. ID No. 450, Seq. ID No. 460, Seq. ID No. 466, Seq. ID No. 486, Seq. ID No. 502, Seq. ID No. 506, Seq. ID No. 512, Seq. ID No. 514, Seq. ID No. 518, Seq. ID No. 520, Seq. ID No. 524, Seq. ID No. 530, Seq. ID No. 534, Seq. ID No. 536, Seq. ID No. 542, Seq. ID No. 544, Seq. ID No. 552, Seq. ID No. 554, Seq. ID No. 560, Seq. ID No. 564, Seq. ID No. 570, Seq. ID No. 576, Seq. ID No. 580, Seq. ID No. 584, Seq. ID No. 592, Seq. ID No. 596, Seq. ID No. 602, Seq. ID No. 606, Seq. ID No. 608, Seq. ID No. 612, Seq. ID No. 614, Seq. ID No. 616, Seq. ID No. 622, Seq. ID No. 628, Seq. ID No. 630, Seq. ID No. 634, Seq. ID No. 636, Seq. ID No. 638, Seq. ID No. 644, Seq. ID No. 650, Seq. ID No. 652, Seq. ID No. 656, Seq. ID No. 658, Seq. ID No. 670, Seq. ID No. 672, Seq. ID No. 676, Seq. ID No. 682, Seq. ID No. 684, Seq. ID No. 686, Seq. ID No. 694, Seq. ID No. 704, Seq. ID No. 706, Seq. ID No. 712, Seq. ID No.;726, Seq. ID No. 732, Seq. ID No. 736, Seq. ID No. 738, Seq. ID No. 742, Seq. ID No. 744, Seq. ID No. 750, Seq. ID No. 754, Seq. ID No. 760, Seq. ID No. 774, Seq. ID No. 782, Seq. ID No. 784, Seq. ID No. 786, Seq. ID No. 790, Seq. ID No. 794, Seq. ID No. 798, Seq. ID No. 804, Seq. ID No. 816, Seq. ID No. 820, Seq. ID No. 822, Seq. ID No. 828, Seq. ID No. 832, Seq. ID No. 834, Seq. ID No. 836, Seq. ID No. 838, Seq. ID No. 842, Seq. ID No. 844, Seq. ID No. 850, Seq. ID No. 852, Seq. ID No. 854, Seq. ID No. 858, Seq. ID No. 866, Seq. ID No. 870, Seq. ID No. 874, Seq. ID No. 878, Seq. ID No. 884, Seq. ID No. 888, Seq. ID No. 892, Seq. ID No. 896, Seq. ID No. 902, Seq. ID No. 908, Seq. ID No. 912, Seq. ID No. 918, Seq. ID No. 922, Seq. ID No. 928, Seq. ID No. 936, Seq. ID No. 940, Seq. ID No. 946, Seq. ID No. 952, Seq. ID No. 954, Seq. ID No. 960, Seq. ID No. 964, Seq. ID No. 968, Seq. ID No. 970, Seq. ID No. 978, Seq. ID No. 990, Seq. ID No. 996, Seq. ID No. 1000, Seq. ID No. 1006, Seq. ID No. 1008, Seq. ID No. 1016, Seq. ID No. 1022, Seq. ID No. 1036, Seq. ID No. 1042, Seq. ID No. 1044, Seq. ID No. 1050, Seq. ID No. 1054, Seq. ID No. 1058, Seq. ID No. 1060, Seq. ID No. 1064, Seq. ID No. 1068, Seq. ID No. 1070, Seq. ID No. 1072, Seq. ID No. 1076, Seq. ID No. 1084, Seq. ID No. 1086, Seq. ID No. 1090, Seq. ID No. 1094, Seq. ID No. 1098, Seq. ID No. 1100, Seq. ID No. 1102, Seq. ID No. 1110, Seq. ID No. 1114, Seq. ID No. 1118, Seq. ID No. 1122, Seq. ID No. 1130, Seq. ID No. 1134, Seq. ID No. 1138, Seq. ID No. 1142, Seq. ID No. 1146, Seq. ID No. 1150, Seq. ID No. 1156, Seq. ID No. 1158, Seq. ID No. 1160, Seq. ID No. 1164, Seq. ID No. 1172, Seq. ID No. 1174, Seq. ID No. 1176, Seq. ID No. 1182, Seq. ID No. 1186, Seq. ID No. 1190, Seq. ID No. 1200, Seq. ID No. 1206, Seq. ID No. 1210, Seq. ID No. 1216, Seq. ID No. 1220, Seq. ID No. 1224, Seq. ID No. 1226, Seq. ID No. 1234, Seq. ID No. 1236, Seq. ID No. 1242, Seq. ID No. 1252, Seq. ID No. 1256, Seq. ID No. 1262, Seq. ID No. 1272, Seq. ID No. 1282, Seq. ID No. 1316, Seq. ID No. 1324, Seq. ID No. 1332, Seq. ID No. 1334, Seq. ID No. 1340, Seq. ID No. 1346, Seq. ID No. 1348, Seq. ID No. 1356, Seq. ID No. 1358, Seq. ID No. 1370, Seq. ID No. 1374, Seq. ID No. 1388, Seq. ID No. 1392, Seq. ID No. 1396, Seq. ID No. 1398, Seq. ID No. 1404, Seq. ID No. 1406, Seq. ID No. 1408, Seq. ID No. 1414, Seq. ID No. 1416, Seq. ID No. 1418, Seq. ID No. 1426, Seq. ID No. 1428, Seq. ID No. 1430, Seq. ID No. 1432, Seq. ID No. 1434, Seq. ID No. 1436, Seq. ID No. 1440, Seq. ID No. 1442, Seq. ID No. 1452, Seq. ID No. 1454, Seq. ID No. 1460, Seq. ID No. 1462, Seq. ID No. 1470, Seq. ID No. 1472, Seq. ID No. 1474, Seq. ID No. 1476, Seq. ID No. 1478, Seq. ID No. 1484, Seq. ID No. 1488, Seq. ID No. 1490, Seq. ID No. 1494, Seq. ID No. 1496, Seq. ID No. 1498, Seq. ID No. 1502, Seq. ID No. 1504, Seq. ID No. 1508, Seq. ID No. 1510, Seq. ID No. 1518, Seq. ID No. 1522, Seq. ID No. 1528, Seq. ID No. 1532, Seq. ID No. 1538, Seq. ID No. 1542, Seq. ID No. 1544, Seq. ID No. 1550, Seq. ID No. 1562, Seq. ID No. 1564, Seq. ID No. 1566, Seq. ID No. 1570, Seq. ID No. 1574, Seq. ID No. 1576, Seq. ID No. 1582, Seq. ID No. 1588, Seq. ID No. 1592, Seq. ID No. 1596, Seq. ID No. 1598, Seq. ID No. 1606, Seq. ID No. 1610, Seq. ID No. 1614, Seq. ID No. 1620, Seq. ID No. 1624, Seq. ID No. 1628, Seq. ID No. 1630, Seq. ID No. 1636, Seq. ID No. 1642, Seq. ID No. 1648, Seq. ID No. 1652, Seq. ID No. 1658, Seq. ID No. 1660, Seq. ID No. 1668, Seq. ID No. 1670, Seq. ID No. 1680, Seq. ID No. 1684, Seq. ID No. 1688, Seq. ID No. 1692, Seq. ID No. 1698, Seq. ID No. 1700, Seq. ID No. 1702, Seq. ID No. 1708, Seq. ID No. 1710, Seq. ID No. 1714, Seq. ID No. 1718, Seq. ID No. 1722, Seq. ID No. 1726, Seq. ID No. 1736, Seq. ID No. 1738, Seq. ID No. 1740, Seq. ID No. 1744, Seq. ID No. 1746, Seq. ID No. 1750, Seq. ID No. 1756, Seq. ID No. 1770, Seq. ID No. 1774, Seq. ID No. 1778, Seq. ID No. 1786, Seq. ID No. 1790, Seq. ID No. 1792, Seq. ID No. 1798, Seq. ID No. 1800, Seq. ID No. 1804, Seq. ID No. 1806, Seq. ID No. 1808, Seq. ID No. 1818, Seq. ID No. 1820, Seq. ID No. 1822, Seq. ID No. 1826, Seq. ID No. 1828, Seq. ID No. 1832, Seq. ID No.-1836, Seq. ID No. 1842, Seq. ID No. 1846, Seq. ID No. 1848, Seq. ID No. 1850, Seq. ID No. 1852, Seq. ID No. 1856, Seq. ID No. 1860, Seq. ID No. 1862, Seq. ID No. 1866, Seq. ID No. 1868, Seq. ID No. 1872, Seq. ID No. 1874, Seq. ID No. 1878, Seq. ID No. 1880, Seq. ID No. 1888, Seq. ID No. 1890, Seq. ID No. 1896, Seq. ID No. 1898, Seq. ID No. 1900, Seq. ID No. 1902, Seq. ID No. 1906, Seq. ID No. 1910, Seq. ID No. 1918, Seq. ID No. 1922, Seq. ID No. 1926, Seq. ID No. 1932, Seq. ID No. 1934, Seq. ID No. 1938, Seq. ID No. 1940, Seq. ID No. 1946, Seq. ID No. 1948, Seq. ID No. 1952, Seq. ID No. 1956, Seq. ID No. 1960, Seq. ID No. 1962, Seq. ID No. 1966, Seq. ID No. 1968, Seq. ID No. 1970, Seq. ID No. 1974, Seq. ID No. 1978, Seq. ID No. 1980, Seq. ID No. 1984, Seq. ID No. 1990, Seq. ID No. 1992, Seq. ID No. 1998, Seq. ID No. 2002, Seq. ID No. 2004, Seq. ID No. 2012, Seq. ID No. 2022, Seq. ID No. 2024, Seq. ID No. 2030, Seq:. ID No. 2034, Seq. ID No. 2038, Seq. ID No. 2040, Seq. ID No. 2046, Seq. ID No. 2048, Seq. ID No. 2052, Seq. ID No. 2058, Seq. ID No. 2062, Seq. ID No. 2068, Seq. ID No. 2070, Seq. ID No. 2072, Seq. ID No. 2074, Seq. ID No. 2078, Seq. ID No. 2090, Seq. ID No. 2094, Seq. ID No. 2096, Seq. ID No. 2100, Seq. ID No. 2102, Seq. ID No. 2114, Seq. ID No. 2118, Seq. ID No. 2124, Seq. ID No. 2136, Seq. ID No. 2138, Seq. ID No. 2140, Seq. ID No. 2144, Seq. ID No. 2148, Seq. ID No. 2154, Seq. ID No. 2160, Seq. ID No. 2164, Seq. ID No. 2168, Seq. ID No. 2176, Seq. ID No. 2182, Seq. ID No. 2186, Seq. ID No. 2190, Seq. ID No. 2194, Seq. ID No. 2196, Seq. ID No. 2198, Seq. ID No. 2206, Seq. ID No. 2218, Seq. ID No. 2220, Seq. ID No. 2224, Seq. ID No. 2226, Seq. ID No. 2230, Seq. ID No. 2236, Seq. ID No. 2240, Seq. ID No. 2244, Seq. ID No. 2246, Seq. ID No. 2248, Seq. ID No. 2254, Seq. ID No. 2256, Seq. ID No. 2258, Seq. ID No. 2260, Seq. ID No. 2262, Seq. ID No. 2266, Seq. ID No. 2270, Seq. ID No. 2274, Seq. ID No. 2286, Seq. ID No. 2298, Seq. ID No. 2300, Seq. ID No. 2302, Seq. ID No. 2314, Seq. ID No. 2328, Seq. ID No. 2332, Seq. ID No. 2336, Seq. ID No. 2338, Seq. ID No. 2342, Seq. ID No. 2344, Seq. ID No. 2346, Seq. ID No. 2350, Seq. ID No. 2352, Seq. ID No. 2358, Seq. ID No. 2360, Seq. ID No. 2362, Seq. ID No. 2366, Seq. ID No. 2370, Seq. ID No. 2374, Seq. ID No. 2378, Seq. ID No. 2384, Seq. ID No. 2398, Seq. ID No. 2402, Seq. ID No. 2404, Seq. ID No. 2410, Seq. ID No. 2412, Seq. ID No. 2418, Seq. ID No. 2422, Seq. ID No. 2424, Seq. ID No. 2426, Seq. ID No. 2430, Seq. ID No. 2436, Seq. ID No. 2438, Seq. ID No. 2440, Seq. ID No. 2442, Seq. ID No. 2446, Seq. ID No. 2450, Seq. ID No. 2452, Seq. ID No. 2454, Seq. ID No. 2462, Seq. ID No. 2466, Seq. ID No. 2468, Seq. ID No. 2472, Seq. ID No. 2474, Seq. ID No. 2478, Seq. ID No. 2480, Seq. ID No. 2482, Seq. ID No. 2486, Seq. ID No. 2488, Seq. ID No. 2496, Seq. ID No. 2506, Seq. ID No. 2508, Seq. ID No. 2510, Seq. ID No. 2520, Seq. ID No. 2526, Seq. ID No. 2528, Seq. ID No. 2530, Seq. ID No. 2536, Seq. ID No. 2538, Seq. ID No. 2540, Seq. ID No. 2548, Seq. ID No. 2552, Seq. ID No. 2564, Seq. ID No. 2566, Seq. ID No. 2574, S6q. ID No. 2578, Seq. ID No. 2580, Seq. ID No. 2586, Seq. ID No. 2594, Seq. ID No. 2596, Seq. ID No. 2600, Seq. ID No. 2608, Seq. ID No. 2610, Seq. ID No. 2614, Seq. ID No. 2616, Seq. ID No. 2620, Seq. ID No. 2624, Seq. ID No. 2626, Seq. ID No. 2632, Seq. ID No. 2634, Seq. ID No. 2640, Seq. ID No. 2642, Seq. ID No. 2644, Seq. ID No. 2648, Seq. ID No. 2652, Seq. ID No. 2656, Seq. ID No. 2658, Seq. ID No. 2662, Seq. ID No. 2666, Seq. ID No. 2670, Seq. ID No. 2672, Seq. ID No. 2674, Seq. ID No. 2682, Seq. ID No. 2690, Seq. ID No. 2694, Seq. ID No. 2700, Seq. ID No. 2704, Seq. ID No. 2710, Seq. ID No. 2712, Seq. ID No. 2714, Seq. ID No. 2720, Seq. ID No. 2722, Seq. ID No. 2728, Seq. ID No. 2738, Seq. ID No. 2742, Seq. ID No. 2746, Seq. ID No. 2750, Seq. ID No. 2754, Seq. ID No. 2758, Seq. ID No. 2762, Seq. ID No. 2766, Seq. ID No. 2768, Seq. ID No. 2770, Seq. ID No. 2774, Seq. ID No. 2776, Seq. ID No. 2778, Seq. ID No. 2788, Seq. ID No. 2790, Seq. ID No. 2792, Seq. ID No. 2794, Seq. ID No. 2796, Seq. ID No. 2800, Seq. ID No. 2804, Seq. ID No. 2816, Seq. ID No. 2818, Seq. ID No. 2820, Seq. ID No. 2826, Seq. ID No. 2844, Seq. ID No. 2848, Seq. ID No. 2850, Seq. ID No. 2852, Seq. ID No. 2858, Seq. ID No. 2860, Seq. ID No. 2864, Seq. ID No. 2868, Seq. ID No. 2870, Seq. ID No. 2872, Seq. ID No. 2878, Seq. ID No. 2880, Seq. ID No. 2890, Seq. ID No. 2892, Seq. ID No. 2898, Seq. ID No. 2902, Seq. ID No. 2904, Seq. ID No. 2908, Seq. ID No. 2910, Seq. ID No. 2914, Seq. ID No. 2916, Seq. ID No. 2920, Seq. ID No. 2922, Seq. ID No. 2924, Seq. ID No. 2926, Seq. ID No. 2930, Seq. ID No. 2936, Seq. ID No. 2944, Seq. ID No. 2952, Seq. ID No. 2956, Seq. ID No. 2964, Seq. ID No. 2966, Seq. ID No. 2972, Seq. ID No. 2974, Seq. ID No. 2976, Seq. ID No. 2980, Seq. ID No. 2982, Seq. ID No. 2986, Seq. ID No. 2990, Seq. ID No. 2994, Seq. ID No. 3000, Seq. ID No. 3002, Seq. ID No. 3004, Seq. ID No. 3018, Seq. ID No. 3028, Seq. ID No. 3034, Seq. ID No. 3036, Seq. ID No. 3040, Seq. ID No. 3042, Seq. ID No. 3044, Seq. ID No. 3046, Seq. ID No. 3048, Seq. ID No. 3056, Seq. ID No. 3058, Seq. ID No. 3062, Seq. ID No. 3066, Seq. ID No. 3070, Seq. ID No. 3080, Seq. ID No. 3090, Seq. ID No. 3092, Seq. ID No. 3094, Seq. ID No. 3096, Seq. ID No. 3104, Seq. ID No. 3106, Seq. ID No. 3110, Seq. ID No. 3114, Seq. ID No. 3126, Seq. ID No. 3128, Seq. ID No. 3136, Seq. ID No. 3138, Seq. ID No. 3142, Seq. ID No. 3144, Seq. ID No. 3148, Seq. ID No. 3156, Seq. ID No. 3158, Seq. ID No. 3160, Seq. ID No. 3162, Seq. ID No. 3166, Seq. ID No. 3170, Seq. ID No. 3176, Seq. ID No. 3180, Seq. ID No. 3184, Seq. ID No. 3186, Seq. ID No. 3190, Seq. ID No. 3192, Seq. ID No. 3196, Seq. ID No. 3200, Seq. ID No. 3206, Seq. ID No. 3212, Seq. ID No. 3218, Seq. ID No. 3222, Seq. ID No. 3226, Seq. ID No. 3230, Seq. ID No. 3232, Seq. ID No. 3238, Seq. ID No. 3240, Seq. ID No. 3248, Seq. ID No. 3252, Seq. ID No. 3254, Seq. ID No. 3256, Seq. ID No. 3260, Seq. ID No. 3262, Seq. ID No. 3268, Seq. ID No. 3274, Seq. ID No. 3278, Seq. ID No. 3280, Seq. ID No. 3286, Seq. ID No. 3288, Seq. ID No. 3290, Seq. ID No. 3294, Seq. ID No. 3296, Seq. ID No. 3300, Seq. ID No. 3302, Seq. ID No. 3306, Seq. ID No. 3310, Seq. ID No. 3316, Seq. ID No. 3320, Seq. ID No. 3322, Seq. ID No. 3328, Seq. ID No. 3334, Seq. ID No. 3338, Seq. ID No. 3344, Seq. ID No. 3354, Seq. ID No. 3360, Seq. ID No. 3362, Seq. ID No. 3366, Seq. ID No. 3374, Seq. ID No. 3382, Seq. ID No. 3386, Seq. ID No. 3394, Seq. ID No. 3402, Seq. ID No. 3408, Seq. ID No. 3412, Seq. ID No. 3416, Seq. ID No. 3418, Seq. ID No. 3424, Seq. ID No. 3430, Seq. ID No. 3438, Seq. ID No. 3440, Seq. ID No. 3444, Seq. ID No. 3460, Seq. ID No. 3462, Seq. ID No. 3466, Seq. ID No. 3470, Seq. ID No. 3472, Seq. ID No. 3474, Seq. ID No. 3476, Seq. ID No. 3478, Seq. ID No. 3484, Seq. ID No. 3488, Seq. ID No. 3496, Seq. ID No. 3498, Seq. ID No. 3506, Seq. ID No. 3508, Seq. ID No. 3510, Seq. ID No. 3512, Seq. ID No. 3516, Seq. ID No. 3522, Seq. ID No. 3524, Seq. ID No. 3530, Seq. ID No. 3536, Seq. ID No. 3542, Seq. ID No. 3552, Seq. ID No. 3560, Seq. ID No. 3564, Seq. ID No. 3568, Seq. ID No. 3576, Seq. ID No. 3578, Seq. ID No. 3584, Seq. ID No. 3586, Seq. ID No. 3590, Seq. ID No. 3592, Seq. ID No. 3600, Seq. ID No. 3602, Seq. ID No. 3608, Seq. ID No. 3610, Seq. ID No. 3612, Seq. ID No. 3616, Seq. ID No. 3620, Seq. ID No. 3630, Seq. ID No. 3632, Seq. ID No. 3634, Seq. ID No. 3638, Seq. ID No. 3642, Seq. ID No. 3644, Seq. ID No. 3646, Seq. ID No. 3652, Seq. ID No. 3654, Seq. ID No. 3656, Seq. ID No. 3662, Seq. ID No. 3666, Seq. ID No. 3668, Seq. ID No. 3670, Seq. ID No. 3676, Seq. ID No. 3682, Seq. ID No. 3690, Seq. ID No. 3696, Seq. ID No. 3698, Seq. ID No. 3706, Seq. ID No. 3712, Seq. ID No. 3720, Seq. ID No. 3726, Seq. ID No. 3730, Seq. ID No. 3732, Seq. ID No. 3736, Seq. ID No. 3740, Seq. ID No. 3742, Seq. ID No. 3744, Seq. ID No. 3750, Seq. ID No. 3760, Seq. ID No. 3770, Seq. ID No. 3774, Seq. ID No. 3778, Seq. ID No. 3782, Seq. ID No. 3788, Seq. ID No. 3796, Seq. ID No. 3802, Seq. ID No. 3806, Seq. ID No. 3810, Seq. ID No. 3814, Seq. ID No. 3820, Seq. ID No. 3822, Seq. ID No. 3826, Seq. ID No. 3828, Seq. ID No. 3830, Seq. ID No. 3832, Seq. ID No. 3836, Seq. ID No. 3842, Seq. ID No. 3846, Seq. ID No. 3856, Seq. ID No. 3858, Seq. ID No. 3864, Seq. ID No. 3868, Seq. ID No. 3870, Seq. ID No. 3876, Seq. ID No. 3882, Seq. ID No. 3884, Seq. ID No. 3886, Seq. ID No. 3888, Seq. ID No. 3898, Seq. ID No. 3902, Seq. ID No. 3906, Seq. ID No. 3910, Seq. ID No. 3914, Seq. ID No. 3916, Seq. ID No. 3918, Seq. ID No. 3920, Seq. ID No. 3922, Seq. ID No. 3930, Seq. ID No. 3932, Seq. ID No. 3936, Seq. ID No. 3942, Seq. ID No. 3944, Seq. ID No. 3946, Seq. ID No. 3948, Seq. ID No. 3952, Seq. ID No. 3954, Seq. ID No. 3958, Seq. ID No. 3972, Seq. ID No. 3978, Seq. ID No. 3982, Seq. ID No. 3986, Seq. ID No. 3988, Seq. ID No. 3990, Seq. ID No. 3994, Seq. ID No. 4002, Seq. ID No. 4006, Seq. ID No. 4012, Seq. ID No. 4016, Seq. ID No. 4018, Seq. ID No. 4020, Seq. ID No. 4024, Seq. ID No. 4030, Seq. ID No. 4032, Seq. ID No. 4034, Seq. ID No. 4038, Seq. ID No. 4040, Seq. ID No. 4050, Seq. ID No. 4052, Seq. ID No. 4058, Seq. ID No. 4060, Seq. ID No. 4066, Seq. ID No. 4070, Seq. ID No. 4076, Seq. ID No. 4080, Seq. ID No. 4086, Seq. ID No. 4090, Seq. ID No. 4096, Seq. ID No. 4098, Seq. ID No. 4100, Seq. ID No. 4106, Seq. ID No. 4112, Seq. ID No. 4116, Seq. ID No. 4122, Seq. ID No. 4124, Seq. ID No. 4128, Seq. ID No. 4130, Seq. ID No. 4132, Seq. ID No. 4138, Seq. ID No. 4142, Seq. ID No. 4148, Seq. ID No. 4158, Seq. ID No. 4160, Seq. ID No. 4164, Seq. ID No. 4166, Seq. ID No. 4170, Seq. ID No. 4180, Seq. ID No. 4184, Seq. ID No. 4188, Seq. ID No. 4194, Seq. ID No. 4196, Seq. ID No. 4198, Seq. ID No. 4200, Seq. ID No. 4202, Seq. ID No. 4206, Seq. ID No. 4208, Seq. ID No. 4214, Seq. ID No. 4220, Seq. ID No. 4236, Seq. ID No. 4238, Seq. ID No. 4240, Seq. ID No. 4244, Seq. ID No. 4246, Seq. ID No. 4256, Seq. ID No. 4260, Seq. ID No. 4268, Seq. ID No. 4272, Seq. ID No. 4278, Seq. ID No. 4280, Seq. ID No. 4282, Seq. ID No. 4284, Seq. ID No. 4290, Seq. ID No. 4294, Seq. ID No. 4300, Seq. ID No. 4302, Seq. ID No. 4304, Seq. ID No. 4306, Seq. ID No. 4308, Seq. ID No. 4310, Seq. ID No. 4314, Seq. ID No. 4316, Seq. ID No. 4324, Seq. ID No. 4326, Seq. ID No. 4328, Seq. ID No. 4332, Seq. ID No. 4334, Seq. ID No. 4338, Seq. ID No. 4342, Seq. ID No. 4346, Seq. ID No. 4350, Seq. ID No. 4356, Seq. ID No. 4358, Seq. ID No. 4366, Seq. ID No. 4368, Seq. ID No. 4372, Seq. ID No. 4376, Seq. ID No. 4378, Seq. ID No. 4380, Seq. ID No. 4384, Seq. ID No. 4388, Seq. ID No. 4394, Seq. ID No. 4398, Seq. ID No. 4402, Seq. ID No. 4410, Seq. ID No. 4414, Seq. ID No. 4422, Seq. ID No. 4434, Seq. ID No. 4436, Seq. ID No. 4446, Seq. ID No. 4450, Seq. ID No. 4458, Seq. ID No. 4462, Seq. ID No. 4464, Seq. ID No. 4472, Seq. ID No. 4476, Seq. ID No. 4478, Seq. ID No. 4482, Seq. ID No. 4488, Seq. ID No. 4492, Seq. ID No. 4500, Seq. ID No. 4502, Seq. ID No. 4504, Seq. ID No. 4510, Seq. ID No. 4518, Seq. ID No. 4532, Seq. ID No. 4534, Seq. ID No. 4536, Seq. ID No. 4540, Seq. ID No. 4542, Seq. ID No. 4546, Seq. ID No. 4550, Seq. ID No. 4558, Seq. ID No. 4564, Seq. ID No. 4568, Seq. ID No. 4570, Seq. ID No. 4576, Seq. ID No. 4578, Seq. ID No. 4580, Seq. ID No. 4584, Seq. ID No. 4592, Seq. ID No. 4594, Seq. ID No. 4598, Seq. ID No. 4602, Seq. ID No. 4606, Seq. ID No. 4610, Seq. ID No. 4612, Seq. ID No. 4614, Seq. ID No. 4624, Seq. ID No. 4626, Seq. ID No. 4630, Seq. ID No. 4634, Seq. ID No. 4638, Seq. ID No. 4642, Seq. ID No. 4646, Seq. ID No. 4652, Seq. ID No. 4654, Seq. ID No. 4656, Seq. ID No. 4662, Seq. ID No. 4664, Seq. ID No. 4670, Seq. ID No. 4672, Seq. ID No. 4674, Seq. ID No. 4676, Seq. ID No. 4686, Seq. ID No. 4690, Seq. ID No. 4698, Seq. ID No. 4704, Seq. ID No. 4706, Seq. ID No. 4714, Seq. ID No. 4718, Seq. ID No. 4720, Seq. ID No. 4724, Seq. ID No. 4730, Seq. ID No. 4732, Seq. ID No. 4734, Seq. ID No. 4740, Seq. ID No. 4744, Seq. ID No. 4746, Seq. ID No. 4748, Seq. ID No. 4750, Seq. ID No. 4752, Seq. ID No. 4754, Seq. ID No. 4762, Seq. ID No. 4768, Seq. ID No. 4772, Seq. ID No. 4780, Seq. ID No. 4782, Seq. ID No. 4784, Seq. ID No. 4786, Seq. ID No. 4790, Seq. ID No. 4792, Seq. ID No. 4794, Seq. ID No. 4800, Seq. ID No. 4808, Seq. ID No. 4810, Seq. ID No. 4812, Seq. ID No. 4814, Seq. ID No. 4816, Seq. ID No. 4822, Seq. ID No. 4826, Seq. ID No. 4828, Seq. ID No. 4830, Seq. ID No. 4838, Seq. ID No. 4840, Seq. ID No. 4846, Seq. ID No. 4848, Seq. ID No. 4850, Seq. ID No. 4858, Seq. ID No. 4862, Seq. ID No. 4866, Seq. ID No. 4868, Seq. ID No. 4876, Seq. ID No. 4880, Seq. ID No. 4882, Seq. ID No. 4884, Seq. ID No. 4886, Seq. ID No. 4888, Seq. ID No. 4894, Seq. ID No. 4900, Seq. ID No. 4902, Seq. ID No. 4904, Seq. ID No. 4906, Seq. ID No. 4908, Seq. ID No. 4910, Seq. ID No. 4916, Seq. ID No. 4918, Seq. ID No. 4920, Seq. ID No. 4922, Seq. ID No. 4926, Seq. ID No. 4930, Seq. ID No. 4934, Seq. ID No. 4938, Seq. ID No. 4942, Seq. ID No. 4944, Seq. ID No. 4946, Seq. ID No. 4948, Seq. ID No. 4954, Seq. ID No. 4956, Seq. ID No. 4960, Seq. ID No. 4962, Seq. ID No. 4966, Seq. ID No. 4968, Seq. ID No. 4972, Seq. ID No. 4974, Seq. ID No. 4978, Seq. ID No. 4986, Seq. ID No. 4990, Seq. ID No. 4992, Seq. ID No. 4996, Seq. ID No. 4998, Seq. ID No. 5006, Seq. ID No. 5010, Seq. ID No. 5016, Seq. ID No. 5020, Seq. ID No. 5026, Seq. ID No. 5030, Seq. ID No. 5038, Seq. ID No. 5046, Seq. ID No. 5050, Seq. ID No. 5054, Seq. ID No. 5056, Seq. ID No. 5060, Seq. ID No. 5062, Seq. ID No. 5066, Seq. ID No. 5072, Seq. ID No. 5074, Seq. ID No. 5080, Seq. ID No. 5082, Seq. ID No. 5084, Seq. ID No. 5088, Seq. ID No. 5090, Seq. ID No. 5094, Seq. ID No. 5096, Seq. ID No. 5104, Seq. ID No. 5106, Seq. ID No. 5108, Seq. ID No. 5112, Seq. ID No. 5114, Seq. ID No. 5116, Seq. ID No. 5118, Seq. ID No. 5120, Seq. ID No. 5142, Seq. ID No. 5144, Seq. ID No. 5148, Seq. ID No. 5154, Seq. ID No. 5156, Seq. ID No. 5164, Seq. ID No. 5170, Seq. ID No. 5172, Seq. ID No. 5174, Seq. ID No. 5180, Seq. ID No. 5186, Seq. ID No. 5188, Seq. ID No. 5194, Seq. ID No. 5196, Seq. ID No. 5202, Seq. ID No. 5204, Seq. ID No. 5206, Seq. ID No. 5210, Seq. ID No. 5212, Seq. ID No. 5214, Seq. ID No. 5218, Seq. ID No. 5226, Seq. ID No. 5232, Seq. ID No. 5234, Seq. ID No. 5238, Seq. ID No. 5240, Seq. ID No. 5246, Seq. ID No. 5248, Seq. ID No. 5250, Seq. ID No. 5256, Seq. ID No. 5258, Seq. ID No. 5260, Seq. ID No. 5264, Seq. ID No. 5270, Seq. ID No. 5274, Seq. ID No. 5284, Seq. ID No. 5290, Seq. ID No. 5294, Seq. ID No. 5308, Seq. ID No. 5312, Seq. ID No. 5314, Seq. ID No. 5316, Seq. ID No. 5324, Seq. ID No. 5326, Seq. ID No. 5336, Seq. ID No. 5338, Seq. ID No. 5340, Seq. ID No. 5346, Seq. ID No. 5348, Seq. ID No. 5350, Seq. ID No. 5354, Seq. ID No. 5362, Seq. ID No. 5366, Seq. ID No. 5368, Seq. ID No. 5370, Seq. ID No. 5374, Seq. ID No. 5376, Seq. ID No. 5388, Seq. ID No. 5390, Seq. ID No. 5392, Seq. ID No. 5394, Seq. ID No. 5396, Seq. ID No. 5398, Seq. ID No. 5400, Seq. ID No. 5402, Seq. ID No. 5404, Seq. ID No. 5406, Seq. ID No. 5412, Seq. ID No. 5416, Seq. ID No. 5418, Seq. ID No. 5420, Seq. ID No. 5422, Seq. ID No. 5424, Seq. ID No. 5430, Seq. ID No. 5434, Seq. ID No. 5442, Seq. ID No. 5444, Seq. ID No. 5446, Seq. ID No. 5450, Seq. ID No. 5452, Seq. ID No. 5454, Seq. ID No. 5456, Seq. ID No. 5458, Seq. ID No. 5470, Seq. ID No. 5476, Seq. ID No. 5484, Seq. ID No. 5486, Seq. ID No. 5490, Seq. ID No. 5494, Seq. ID No. 5500, Seq. ID No. 5502, Seq. ID No. 5506, Seq. ID No. 5510, Seq. ID No. 5512, Seq. ID No. 5524, Seq. ID No. 5530, Seq. ID No. 5536, Seq. ID No. 5540, Seq. ID No. 5542, Seq. ID No. 5548, Seq. ID No. 5552, Seq. ID No. 5556, Seq. ID No. 5560, Seq. ID No. 5562, Seq. ID No. 5566, Seq. ID No. 5572, Seq. ID No. 5574, Seq. ID No. 5580, Seq. ID No. 5584, Seq. ID No. 5590, Seq. ID No. 5600, Seq. ID No. 5602, Seq. ID No. 5604, Seq. ID No. 5614, Seq. ID No. 5622, Seq. ID No. 5628, Seq. ID No. 5632, Seq. ID No. 5636, Seq. ID No. 5646, Seq. ID No. 5652, Seq. ID No. 5654, Seq. ID No. 5658, Seq. ID No. 5662, Seq. ID No. 5664, Seq. ID No. 5666, Seq. ID No. 5670, Seq. ID No. 5672, Seq. ID No. 5674, Seq. ID No. 5676, Seq. ID No. 5682, Seq. ID No. 5694, Seq. ID No. 5696, Seq. ID No. 5700, Seq. ID No. 5702, Seq. ID No. 5708, Seq. ID No. 5714, Seq. ID No. 5718, Seq. ID No. 5724, Seq. ID No. 5726, Seq. ID No. 5732, Seq. ID No. 5738, Seq. ID No. 5748, Seq. ID No. 5752, Seq. ID No. 5756, Seq. ID No. 5760, Seq. ID No. 5772, Seq. ID No. 5774, Seq. ID No. 5778, Seq. ID No. 5780, Seq. ID No. 5782, Seq. ID No. 5786, Seq. ID No. 5794, Seq. ID No. 5800, Seq. ID No. 5806, Seq. ID No. 5812, Seq. ID No. 5816, Seq. ID No. 5818, Seq. ID No. 5824, Seq. ID No. 5832, Seq. ID No. 5836, Seq. ID No. 5840, Seq. ID No. 5844, Seq. ID No. 5848, Seq. ID No. 5850, Seq. ID No. 5852, Seq. ID No. 5854, Seq. ID No. 5856, Seq. ID No. 5858, Seq. ID No. 5862, Seq. ID No. 5868, Seq. ID No. 5876, Seq. ID No. 5878, Seq. ID No. 5882, Seq. ID No. 5890, Seq. ID No. 5896, Seq. ID No. 5898, Seq. ID No. 5902, Seq. ID No. 5904, Seq. ID No. 5908, Seq. ID No. 5914, Seq. ID No. 5916, Seq. ID No. 5918, Seq. ID No. 5920, Seq. ID No. 5926, Seq. ID No. 5932, Seq. ID No. 5934, Seq. ID No. 5936, Seq. ID No. 5942, Seq. ID No. 5944, Seq. ID No. 5946, Seq. ID No. 5950, Seq. ID No. 5956, Seq. ID No. 5958, Seq. ID No. 5960, Seq. ID No. 5968, Seq. ID No. 5972, Seq. ID No. 5976, Seq. ID No. 5978, Seq. ID No. 5982, Seq. ID No. 5984, Seq. ID No. 5986, Seq. ID No. 5990, Seq. ID No. 5992, Seq. ID No. 5996, Seq. ID No. 5998, Seq. ID No. 6004, Seq. ID No. 6006, Seq. ID No. 6008, Seq. ID No. 6016, Seq. ID No. 6018, Seq. ID No. 6020, Seq. ID No. 6024, Seq. ID No. 6030, Seq. ID No. 6034, Seq. ID No. 6036, Seq. ID No. 6038, Seq. ID No. 6044, Seq. ID No. 6050, Seq. ID No. 6056, Seq. ID No. 6066, Seq. ID No. 6070, Seq. ID No. 6076, Seq-. ID No. 6082, Seq. ID No. 6084, Seq. ID No. 6088, Seq. ID No. 6096, Seq. ID No. 6098, Seq. ID No. 6100, Seq. ID No. 6104, Seq. ID No. 6106, Seq. ID No. 6114, Seq. ID No. 6116, Seq. ID No. 6120, Seq. ID No. 6126, Seq. ID No. 6128, Seq. ID No. 6130, Seq. ID No. 6132, Seq. ID No. 6134, Seq. ID No. 6142, Seq. ID No. 6144, Seq. ID No. 6148, Seq. ID No. 6154, Seq. ID No. 6166, Seq. ID No. 6170, Seq. ID No. 6172, Seq. ID No. 6174, Seq. ID No. 6178, Seq. ID No. 6180, Seq. ID No. 6188, Seq. ID No. 6194, Seq. ID No. 6202, Seq. ID No. 6208, Seq. ID No. 6212, Seq. ID No. 6216, Seq. ID No. 6220, Seq. ID No. 6232, Seq. ID No. 6236, Seq. ID No. 6244, Seq. ID No. 6248, Seq. ID No. 6252, Seq. ID No. 6256, Seq. ID No. 6262, Seq. ID No. 6264, Seq. ID No. 6270, Seq. ID No. 6276, Seq. ID No. 6278, Seq. ID No. 6280, Seq. ID No. 6288, Seq. ID No. 6292, Seq. ID No. 6300, Seq. ID No. 6306, Seq. ID No. 6308, Seq. ID No. 6314, Seq. ID No. 6316, Seq. ID No. 6322, Seq. ID No. 6324, Seq. ID No. 6326, Seq. ID No. 6332, Seq. ID No. 6336, Seq. ID No. 6340, Seq. ID No. 6342, Seq. ID No. 6344, Seq. ID No. 6346, Seq. ID No. 6348, Seq. ID No. 6356, Seq. ID No. 6360, Seq. ID No. 6362, Seq. ID No. 6366, Seq. ID No. 6370, Seq. ID No. 6372, Seq. ID No. 6376, Seq. ID No. 6378, Seq. ID No. 6380, Seq. ID No. 6388, Seq. ID No. 6392, Seq. ID No. 6398, Seq. ID No. 6402, Seq. ID No. 6406, Seq. ID No. 6410, Seq. ID No. 6414, Seq. ID No. 6418, Seq. ID No. 6420, Seq. ID No. 6424, Seq. ID No. 6430, Seq. ID No. 6434, Seq. ID No. 6436, Seq. ID No. 6440, Seq. ID No. 6446, Seq. ID No. 6448, Seq. ID No. 6452, Seq. ID No. 6456, Seq. ID No. 6460, Seq. ID No. 6462, Seq. ID No. 6464, Seq. ID No. 6476, Seq. ID No. 6482, Seq. ID No. 6484, Seq. ID No. 6486, Seq. ID No. 6488, Seq. ID No. 6490, Seq. ID No. 6492, Seq. ID No. 6494, Seq. ID No. 6496, Seq. ID No. 6500, Seq. ID No. 6506, Seq. ID No. 6510, Seq. ID No. 6514, Seq. ID No. 6520, Seq. ID No. 6522, Seq. ID No. 6526, Seq. ID No. 6528, Seq. ID No. 6532, Seq. ID No. 6534, Seq. ID No. 6536, Seq. ID No. 6546, Seq. ID No. 6552, Seq. ID No. 6554, Seq. ID No. 6560, Seq. ID No. 6562, Seq. ID No. 6564, Seq. ID No. 6566, Seq. ID No. 6568, Seq. ID No. 6570, Seq. ID No. 6576, Seq. ID No. 6580, Seq. ID No. 6584, Seq. ID No. 6586, Seq. ID No. 6592, Seq. ID No. 6596, Seq. ID No. 6606, Seq. ID No. 6608, Seq. ID No. 6612, Seq. ID No. 6616, Seq. ID No. 6618, Seq. ID No. 6620, Seq. ID No. 6624, Seq. ID No. 6626, Seq. ID No. 6630, Seq. ID No. 6634, Seq. ID No. 6636, Seq. ID No. 6642, and Seq. ID No. 6648.

54. The Alloiococcus otitidis polypeptide of claim 36, wherein the polypeptide is identified as an Alloiococcus otitidis polypeptide identified by an ORF finder program that searches for an ATG, GTG or TTG start codon between a Stop-Stop region, and is selected from one of Seq. ID No. 2, Seq. ID No. 6, Seq. ID No. 12, Seq. ID No. 14, Seq. ID No. 16, Seq. ID No. 18, Seq. ID No. 20, Seq. ID No. 22, Seq. ID No. 24, Seq. ID No. 28, Seq. ID No. 30, Seq. ID No. 32, Seq. ID No. 36, Seq. ID No. 38, Seq. ID No. 42, Seq. ID No. 44, Seq. ID No. 46, Seq. ID No. 48, Seq. ID No. 50, Seq. ID No. 52, Seq. ID No. 54, Seq. ID No. 56, Seq. ID No. 58, Seq. ID No. 60, Seq. ID No. 66, Seq. ID No. 68, Seq. ID No. 76, Seq. ID No. 78, Seq. ID No. 80, Seq. ID No. 82, Seq. ID No. 84, Seq. ID No. 90, Seq. ID No. 92, Seq. ID No. 94, Seq. ID No. 98, Seq. ID No. 100, Seq. ID No. 104, Seq. ID No. 106, Seq. ID No. 108, Seq. ID No. 110, Seq. ID No. 112, Seq. ID No. 114, Seq. ID No. 116, Seq. ID No. 118, Seq. ID No. 120, Seq. ID No. 122, Seq. ID No. 128, Seq. ID No. 130, Seq. ID No. 132, Seq. ID No. 134, Seq. ID No. 136, Seq. ID No. 138, Seq. ID No. 140, Seq. ID No. 144, Seq. ID No. 146, Seq. ID No. 148, Seq. ID No. 150, Seq. ID No. 154, Seq. ID No. 156, Seq. ID No. 158, Seq. ID No. 160, Seq. ID No. 162, Seq. ID No. 164, Seq. ID No. 166, Seq. ID No. 168, Seq. ID No. 170, Seq. ID No. 172, Seq. ID No. 176, Seq. ID No. 178, Seq. ID No. 180, Seq. ID No. 182, Seq. ID No. 184, Seq. ID No. 188, Seq. ID No. 192, Seq. ID No. 194, Seq. ID No. 196, Seq. ID No. 202, Seq. ID No. 206, Seq. ID No. 208, Seq. ID No. 216, Seq. ID No. 220, Seq. ID No. 222, Seq. ID No. 224, Seq. ID No. 226, Seq. ID No. 228, Seq. ID No. 232, Seq. ID No. 234, Seq. ID No. 240, Seq. ID No. 244, Seq. ID No. 246, Seq. ID No. 248, Seq. ID No. 254, Seq. ID No. 258, Seq. ID No. 260, Seq. ID No. 262, Seq. ID No. 264, Seq. ID No. 266, Seq. ID No. 268, Seq. ID No. 270, Seq. ID No. 274, Seq. ID No. 278, Seq. ID No. 280, Seq. ID No. 282, Seq. ID No. 284, Seq. ID No. 292, Seq. ID No. 294, Seq. ID No. 298, Seq. ID No. 302, Seq. ID No. 306, Seq. ID No. 308, Seq. ID No. 310, Seq. ID No. 314, Seq. ID No. 318, Seq. ID No. 322, Seq. ID No. 324, Seq. ID No. 326, Seq. ID No. 332, Seq. ID No. 334, Seq. ID No. 336, Seq. ID No. 338, Seq. ID No. 340, Seq. ID No. 344, Seq. ID No. 346, Seq. ID No. 348, Seq. ID No. 350, Seq. ID No. 354, Seq. ID No. 356, Seq. ID No. 358, Seq. ID No. 360, Seq. ID No. 362, Seq. ID No. 364, Seq. ID No. 366, Seq. ID No. 368, Seq. ID No. 370, Seq. ID No. 372, Seq. ID No. 376, Seq. ID No. 378, Seq. ID No. 380, Seq. ID No. 384, Seq. ID No. 386, Seq. ID No. 388, Seq. ID No. 390, Seq. ID No. 392, Seq. ID No. 394, Seq. ID No. 398, Seq. ID No. 402, Seq. ID No. 404, Seq. ID No. 406, Seq. ID No. 408, Seq. ID No. 410, Seq. ID No. 416, Seq. ID No. 424, Seq. ID No. 426, Seq. ID No. 428, Seq. ID No. 430, Seq. ID No. 432, Seq. ID No. 434, Seq. ID No. 436, Seq. ID No. 438, Seq. ID No. 440, Seq. ID No. 442, Seq. ID No. 444, Seq. ID No. 446, Seq. ID No. 448, Seq. ID No. 452, Seq. ID No. 454, Seq. ID No. 456, Seq. ID No. 458, Seq. ID No. 462, Seq. ID No. 464, Seq. ID No. 468, Seq. ID No. 470, Seq. ID No. 472, Seq. ID No. 474, Seq. ID No. 476, Seq. ID No. 478, Seq. ID No. 480, Seq. ID No. 482, Seq. ID No. 484, Seq. ID No. 486, Seq. ID No. 490, Seq. ID No. 492, Seq. ID No. 494, Seq. ID No. 496, Seq. ID No. 502, Seq. ID No. 506, Seq. ID No. 508, Seq. ID No. 512, Seq. ID No. 516, Seq. ID No. 518, Seq. ID No. 524, Seq. ID No. 530, Seq. ID No. 532, Seq. ID No. 534, Seq. ID No. 538, Seq. ID No. 540, Seq. ID No. 544, Seq. ID No. 548, Seq. ID No. 550, Seq. ID No. 552, Seq. ID No. 554, Seq. ID No. 556, Seq. ID No. 558, Seq. ID No. 560, Seq. ID No. 562, Seq. ID No. 566, Seq. ID No. 570, Seq. ID No. 572, Seq. ID No. 574, Seq. ID No. 576, Seq. ID No. 580, Seq. ID No. 582, Seq. ID No. 586, Seq. ID No. 588, Seq. ID No. 590, Seq. ID No. 592, Seq. ID No. 594, Seq. ID No. 596, Seq. ID No. 598, Seq. ID No. 600, Seq. ID No. 602, Seq. ID No. 604, Seq. ID No. 606, Seq. ID No. 610, Seq. ID No. 614, Seq. ID No. 616, Seq. ID No. 618, Seq. ID No. 620, Seq. ID No. 622, Seq. ID No. 624, Seq. ID No. 626, Seq. ID No. 628, Seq. ID No. 636, Seq. ID No. 638, Seq. ID No. 644, Seq. ID No. 646, Seq. ID No. 648, Seq. ID No. 650, Seq. ID No. 656, Seq. ID No. 664, Seq. ID No. 666, Seq. ID No. 668, Seq. ID No. 670, Seq. ID No. 676, Seq. ID No. 682, Seq. ID No. 684, Seq. ID No. 688, Seq. ID No. 690, Seq. ID No. 692, Seq. ID No. 696, Seq. ID No. 698, Seq. ID No. 700, Seq. ID No. 702, Seq. ID No. 704, Seq. ID No. 706, Seq. ID No. 708, Seq. ID No. 710, Seq. ID No. 714, Seq. ID No. 726, Seq. ID No. 728, Seq. ID No. 730, Seq. ID No. 732, Seq. ID No. 734, Seq. ID No. 736, Seq. ID No. 740, Seq. ID No. 742, Seq. ID No. 746, Seq. ID No. 748, Seq. ID No. 750, Seq. ID No. 752, Seq. ID No. 754, Seq. ID No. 760, Seq. ID No. 764, Seq. ID No. 766, Seq. ID No. 774, Seq. ID No. 776, Seq. ID No. 782, Seq. ID No. 784, Seq. ID No. 788, Seq. ID No. 794, Seq. ID No. 796, Seq. ID No. 800, Seq. ID No. 802, Seq. ID No. 806, Seq. ID No. 808, Seq. ID No. 810, Seq. ID No. 812, Seq. ID No. 814, Seq. ID No. 816, Seq. ID No. 818, Seq. ID No. 820, Seq. ID No. 822, Seq. ID No. 824, Seq. ID No. 828, Seq. ID No. 830, Seq. ID No. 832, Seq. ID No. 836, Seq. ID No. 838, Seq. ID No. 840, Seq. ID No. 844, Seq. ID No. 846, Seq. ID No. 848, Seq. ID No. 856, Seq. ID No. 860, Seq. ID No. 862, Seq. ID No. 866, Seq. ID No. 868, Seq. ID No. 872, Seq. ID No. 874, Seq. ID No. 876, Seq. ID No. 878, Seq. ID No. 880, Seq. ID No. 882, Seq. ID No. 886, Seq. ID No. 896, Seq. ID No. 898, Seq. ID No. 900, Seq. ID No. 902, Seq. ID No. 904, Seq. ID No. 906, Seq. ID No. 908, Seq. ID No. 910, Seq. ID No. 914, Seq. ID No. 916, Seq. ID No. 922, Seq. ID No. 924, Seq. ID No. 926, Seq. ID No. 932, Seq. ID No. 934, Seq. ID No. 936, Seq. ID No. 938, Seq. ID No. 940, Seq. ID No. 942, Seq. ID No. 944, Seq. ID No. 948, Seq. ID No. 950, Seq. ID No. 952, Seq. ID No. 954, Seq. ID No. 956, Seq. ID No. 958, Seq. ID No. 962, Seq. ID No. 964, Seq. ID No. 966, Seq. ID No. 968, Seq. ID No. 970, Seq. ID No. 972, Seq. ID No. 974, Seq. ID No. 976, Seq. ID No. 982, Seq. ID No. 984, Seq. ID No. 986, Seq. ID No. 988, Seq. ID No. 990, Seq. ID No. 992, Seq. ID No. 994, Seq. ID No. 996, Seq. ID No. 998, Seq. ID No. 1002, Seq. ID No. 1004, Seq. ID No. 1006, Seq. ID No. 1008, Seq. ID No. 1010, Seq. ID No. 1018, Seq. ID No. 1020, Seq. ID No. 1024, Seq. ID No. 1026, Seq. ID No. 1028, Seq. ID No. 1040, Seq. ID No. 1042, Seq. ID No. 1044, Seq. ID No. 1046, Seq. ID No. 1048, Seq. ID No. 1050, Seq. ID No. 1052, Seq. ID No. 1058, Seq. ID No. 1060, Seq. ID No. 1062, Seq. ID No. 1066, Seq. ID No. 1070, Seq. ID No. 1076, Seq. ID No. 1078, Seq. ID No. 1080, Seq. ID No. 1082, Seq. ID No. 1084, Seq. ID No. 1086, Seq. ID No. 1088, Seq. ID No. 1092, Seq. ID No. 1094, Seq. ID No. 1096, Seq. ID No. 1098, Seq. ID No. 1100, Seq. ID No. 1102, Seq. ID No. 1104, Seq. ID No. 1108, Seq. ID No. 1110, Seq. ID No. 1112, Seq. ID No. 1114, Seq. ID No. 1116, Seq. ID No. 1120, Seq. ID No. 1122, Seq. ID No. 1124, Seq. ID No. 1126, Seq. ID No. 1128, Seq. ID No. 1130, Seq. ID No. 1132, Seq. ID No. 1138, Seq. ID No. 1140, Seq. ID No. 1142, Seq. ID No. 1144, Seq. ID No. 1146, Seq. ID No. 1148, Seq. ID No. 1150, Seq. ID No. 1152, Seq. ID No. 1154, Seq. ID No. 1156, Seq. ID No. 1158, Seq. ID No. 1160, Seq. ID No. 1162, Seq. ID No. 1164, Seq. ID No. 1172, Seq. ID No. 1176, Seq. ID No. 1178, Seq. ID No. 1180, Seq. ID No. 1182, Seq. ID No. 1188, Seq. ID No. 1192, Seq. ID No. 1194, Seq. ID No. 1196, Seq. ID No. 1198, Seq. ID No. 1202, Seq. ID No. 1204, Seq. ID No. 1206, Seq. ID No. 1208, Seq. ID No. 1212, Seq. ID No. 1214, Seq. ID No. 1216, Seq. ID No. 1218, Seq. ID No. 1222, Seq. ID No. 1224, Seq. ID No. 1226, Seq. ID No. 1232, Seq. ID No. 1234, Seq. ID No. 1238, Seq. ID No. 1240, Seq. ID No. 1242, Seq. ID No. 1246, Seq. ID No. 1248, Seq. ID No. 1250, Seq. ID No. 1252, Seq. ID No. 1254, Seq. ID No. 1256, Seq. ID No. 1258, Seq. ID No. 1260, Seq. ID No. 1262, Seq. ID No. 1264, Seq. ID No. 1272, Seq. ID No. 1274, Seq. ID No. 1276, Seq. ID No. 1278, Seq. ID No. 1280, Seq. ID No. 1286, Seq. ID No. 1290, Seq. ID No. 1292, Seq. ID No. 1294, Seq. ID No. 1296, Seq. ID No. 1298, Seq. ID No. 1300, Seq. ID No. 1302, Seq. ID No. 1304, Seq. ID No. 1306, Seq. ID No. 1308, Seq. ID No. 1310, Seq. ID No. 1312, Seq. ID No. 1314, Seq. ID No. 1318, Seq. ID No. 1320, Seq. ID No. 1322, Seq. ID No. 1332, Seq. ID No. 1334, Seq. ID No. 1336, Seq. ID No. 1338, Seq. ID No. 1340, Seq. ID No. 1344, Seq. ID No. 1346, Seq. ID No. 1348, Seq. ID No. 1350, Seq. ID No. 1352, Seq. ID No. 1360, Seq. ID No. 1362, Seq. ID No. 1364, Seq. ID No. 1366, Seq. ID No. 1370, Seq. ID No. 1372, Seq. ID No. 1376, Seq. ID No. 1378, Seq. ID No. 1382, Seq. ID No. 1384, Seq. ID No. 1386, Seq. ID No. 1388, Seq. ID No. 1390, Seq. ID No. 1392, Seq. ID No. 1394, Seq. ID No. 1402, Seq. ID No. 1404, Seq. ID No. 1408, Seq. ID No. 1410, Seq. ID No. 1412, Seq. ID No. 1414, Seq. ID No. 1416, Seq. ID No. 1420, Seq. ID No. 1422, Seq. ID No. 1424, Seq. ID No. 1426, Seq. ID No. 1428, Seq. ID No. 1430, Seq. ID No. 1434, Seq. ID No. 1438, Seq. ID No. 1442, Seq. ID No. 1444, Seq. ID No. 1448, Seq. ID No. 1450, Seq. ID No. 1452, Seq. ID No. 1456, Seq. ID No. 1460, Seq. ID No. 1464, Seq. ID No. 1466, Seq. ID No. 1468, Seq. ID No. 1470, Seq. ID No. 1472, Seq. ID No. 1474, Seq. ID No. 1476, Seq. ID No. 1478, Seq. ID No. 1480, Seq. ID No. 1484, Seq. ID No. 1486, Seq. ID No. 1488, Seq. ID No. 1492, Seq. ID No. 1494, Seq. ID No. 1496, Seq. ID No. 1498, Seq. ID No. 1500, Seq. ID No. 1502, Seq. ID No. 1506, Seq. ID No. 1508, Seq. ID No. 1512, Seq. ID No. 1518, Seq. ID No. 1520, Seq. ID No. 1524, Seq. ID No. 1526, Seq. ID No. 1528, Seq. ID No. 1532, Seq. ID No. 1540, Seq. ID No. 1544, Seq. ID No. 1550, Seq. ID No. 1552, Seq. ID No. 1554, Seq. ID No. 1556, Seq. ID No. 1558, Seq. ID No. 1562, Seq. ID No. 1564, Seq. ID No. 1566, Seq. ID No. 1568, Seq. ID No. 1572, Seq. ID No. 1574, Seq. ID No. 1576, Seq. ID No. 1578, Seq. ID No. 1580, Seq. ID No. 1582, Seq. ID No. 1590, Seq. ID No. 1594, Seq. ID No. 1596, Seq. ID No. 1598, Seq. ID No. 1600, Seq. ID No. 1602, Seq. ID No. 1608, Seq. ID No. 1612, Seq. ID No. 1614, Seq. ID No. 1616, Seq. ID No. 1618, Seq. ID No. 1620, Seq. ID No. 1624, Seq. ID No. 1626, Seq. ID No. 1628, Seq. ID No. 1634, Seq. ID No. 1638, Seq. ID No. 1640, Seq. ID No. 1642, Seq. ID No. 1644, Seq. ID No. 1648, Seq. ID No. 1652, Seq. ID No. 1654, Seq. ID No. 1656, Seq. ID No. 1658, Seq. ID No. 1662, Seq. ID No. 1664, Seq. ID No. 1666, Seq. ID No. 1670, Seq. ID No. 1676, Seq. ID No. 1680, Seq. ID No. 1682, Seq. ID No. 1686, Seq. ID No. 1688, Seq. ID No. 1690, Seq. ID No. 1692, Seq. ID No. 1694, Seq. ID No. 1696, Seq. ID No. 1698, Seq. ID No. 1700, Seq. ID No. 1704, Seq. ID No. 1706, Seq. ID No. 1708, Seq. ID No. 1712, Seq. ID No. 1714, Seq. ID No. 1716, Seq. ID No. 1718, Seq. ID No. 1720, Seq. ID No. 1722, Seq. ID No. 1724, Seq. ID No. 1728, Seq. ID No. 1730, Seq. ID No. 1736, Seq. ID No. 1738, Seq. ID No. 1740, Seq. ID No. 1742, Seq. ID No. 1748, Seq. ID No. 1750, Seq. ID No. 1752, Seq. ID No. 1756, Seq. ID No. 1758, Seq. ID No. 1760, Seq. ID No. 1764, Seq. ID No. 1770, Seq. ID No. 1772, Seq. ID No. 1776, Seq. ID No. 1778, Seq. ID No. 1780, Seq. ID No. 1782, Seq. ID No. 1784, Seq. ID No. 1786, Seq. ID No. 1790, Seq. ID No. 1792, Seq. ID No. 1794, Seq. ID No. 1796, Seq. ID No. 1798, Seq. ID No. 1806, Seq. ID No. 1810, Seq. ID No. 1812, Seq. ID No. 1814, Seq. ID No. 1816, Seq. ID No. 1818, Seq. ID No. 1820, Seq. ID No. 1822, Seq. ID No. 1824, Seq. ID No. 1826, Seq. ID No. 1830, Seq. ID No. 1834, Seq. ID No. 1842, Seq. ID No. 1844, Seq. ID No. 1846, Seq. ID No. 1848, Seq. ID No. 1850, Seq. ID No. 1854, Seq. ID No. 1856, Seq. ID No. 1858, Seq. ID No. 1860, Seq. ID No. 1862, Seq. ID No. 1864, Seq. ID No. 1866, Seq. ID No. 1868, Seq. ID No. 1872, Seq. ID No. 1874, Seq. ID No. 1876, Seq. ID No. 1878, Seq. ID No. 1882, Seq. ID No. 1884, Seq. ID No. 1886, Seq. ID No. 1888, Seq. ID No. 1890, Seq. ID No. 1896, Seq. ID No. 1904, Seq. ID No. 1906, Seq. ID No. 1908, Seq. ID No. 1912, Seq. ID No. 1914, Seq. ID No. 1916, Seq. ID No. 1920, Seq. ID No. 1924, Seq. ID No. 1926, Seq. ID No. 1928, Seq. ID No. 1930, Seq. ID No. 1932, Seq. ID No. 1936, Seq. ID No. 1942, Seq. ID No. 1944, Seq. ID No. 1946, Seq. ID No. 1948, Seq. ID No. 1950, Seq. ID No. 1952, Seq. ID No. 1954, Seq. ID No. 1960, Seq. ID No. 1962, Seq. ID No. 1964, Seq. ID No. 1966, Seq. ID No. 1968, Seq. ID No. 1970, Seq. ID No. 1972, Seq. ID No. 1980, Seq. ID No. 1984, Seq. ID No. 1986, Seq. ID No. 1988, Seq. ID No. 1990, Seq. ID No. 1992, Seq. ID No. 1996, Seq. ID No. 1998, Seq. ID No. 2000, Seq. ID No. 2002, Seq. ID No. 2004, Seq. ID No. 2006, Seq. ID No. 2008, Seq. ID No. 2016, Seq. ID No. 2020, Seq. ID No. 2022, Seq. ID No. 2026, Seq. ID No. 2028, Seq. ID No. 2030, Seq. ID No. 2032, Seq. ID No. 2036, Seq. ID No. 2038, Seq. ID No. 2042, Seq. ID No. 2044, Seq. ID No. 2046, Seq. ID No. 2048, Seq. ID No. 2054, Seq. ID No. 2056, Seq. ID No. 2060, Seq. ID No. 2064, Seq. ID No. 2066, Seq. ID No. 2068, Seq. ID No. 2070, Seq. ID No. 2072, Seq. ID No. 2074, Seq. ID No. 2076, Seq. ID No. 2078, Seq. ID No. 2080, Seq. ID No. 2082, Seq. ID No. 2084, Seq. ID No. 2086, Seq. ID No. 2090, Seq. ID No. 2092, Seq. ID No. 2094, Seq. ID No. 2098, Seq. ID No. 2100, Seq. ID No. 2102, Seq. ID No. 2104, Seq. ID No. 2108, Seq. ID No. 2110, Seq. ID No. 2112, Seq. ID No. 2116, Seq. ID No. 2118, Seq. ID No. 2120, Seq. ID No. 2126, Seq. ID No. 2128, Seq. ID No. 2130, Seq. ID No. 2132, Seq. ID No. 2134, Seq. ID No. 2136, Seq. ID No. 2138, Seq. ID No. 2142, Seq. ID No. 2144, Seq. ID No. 2146, Seq. ID No. 2150, Seq. ID No. 2152, Seq. ID No. 2154, Seq. ID No. 2156, Seq. ID No. 2158, Seq. ID No. 2160, Seq. ID No. 2162, Seq. ID No. 2166, Seq. ID No. 2168, Seq. ID No. 2170, Seq. ID No. 2180, Seq. ID No. 2182, Seq. ID No. 2184, Seq. ID No. 2190, Seq. ID No. 2192, Seq. ID No. 2196, Seq. ID No. 2198, Seq. ID No. 2200, Seq. ID No. 2202, Seq. ID No. 2204, Seq. ID No. 2206, Seq. ID No. 2208, Seq. ID No. 2210, Seq. ID No. 2212, Seq. ID No. 2214, Seq. ID No. 2216, Seq. ID No. 2218, Seq. ID No. 2222, Seq. ID No. 2224, Seq. ID No. 2228, Seq. ID No. 2230, Seq. ID No. 2236, Seq. ID No. 2242, Seq. ID No. 2246, Seq. ID No. 2248, Seq. ID No. 2252, Seq. ID No. 2254, Seq. ID No. 2258, Seq. ID No. 2260, Seq. ID No. 2262, Seq. ID No. 2264, Seq. ID No. 2266, Seq. ID No. 2268, Seq. ID No. 2270, Seq. ID No. 2276, Seq. ID No. 2278, Seq. ID No. 2280, Seq. ID No. 2286, Seq. ID No. 2288, Seq. ID No. 2298, Seq. ID No. 2300, Seq. ID No. 2302, Seq. ID No. 2310, Seq. ID No. 2312, Seq. ID No. 2316, Seq. ID No. 2328, Seq. ID No. 2330, Seq. ID No. 2334, Seq. ID No. 2344, Seq. ID No. 2346, Seq. ID No. 2350, Seq. ID No. 2352, Seq. ID No. 2358, Seq. ID No. 2364, Seq. ID No. 2366, Seq. ID No. 2368, Seq. ID No. 2370, Seq. ID No. 2372, Seq. ID No. 2374, Seq. ID No. 2376, Seq. ID No. 2380, Seq. ID No. 2382, Seq. ID No. 2386, Seq. ID No. 2388, Seq. ID No. 2392, Seq. ID No. 2394, Seq. ID No. 2396, Seq. ID No. 2398, Seq. ID No. 2400, Seq. ID No. 2402, Seq. ID No. 2406, Seq. ID No. 2410, Seq. ID No. 2414, Seq. ID No. 2416, Seq. ID No. 2418, Seq. ID No. 2422, Seq. ID No. 2424, Seq. ID No. 2426, Seq. ID No. 2428, Seq. ID No. 2430, Seq. ID No. 2436, Seq. ID No. 2438, Seq. ID No. 2446, Seq. ID No. 2448, Seq. ID No. 2450, Seq. ID No. 2452, Seq. ID No. 2454, Seq. ID No. 2456, Seq. ID No. 2458, Seq. ID No. 2460, Seq. ID No. 2462, Seq. ID No. 2464, Seq. ID No. 2466, Seq. ID No. 2470, Seq. ID No. 2472, Seq. ID No. 2476, Seq. ID No. 2482, Seq. ID No. 2484, Seq. ID No. 2486, Seq. ID No. 2492, Seq. ID No. 2494, Seq. ID No. 2496, Seq. ID No. 2500, Seq. ID No. 2502, Seq. ID No. 2504, Seq. ID No. 2506, Seq. ID No. 2508, Seq. ID No. 2512, Seq. ID No. 2514, Seq. ID No. 2518, Seq. ID No. 2520, Seq. ID No. 2522, Seq. ID No. 2526, Seq. ID No. 2530, Seq. ID No. 2534, Seq. ID No. 2536, Seq. ID No. 2538, Seq. ID No. 2540, Seq. ID No. 2548, Seq. ID No. 2550, Seq. ID No. 2552, Seq. ID No. 2554, Seq. ID No. 2560, Seq. ID No. 2562, Seq. ID No. 2564, Seq. ID No. 2566, Seq. ID No. 2574, Seq. ID No. 2576, Seq. ID No. 2578, Seq. ID No. 2580, Seq. ID No. 2582, Seq. ID No. 2588, Seq. ID No. 2590, Seq. ID No. 2592, Seq. ID No. 2594, Seq. ID No. 2596, Seq. ID No. 2600, Seq. ID No. 2604, Seq. ID No. 2606, Seq. ID No. 2608, Seq; ID No. 2610, Seq. ID No. 2612, Seq. ID No. 2614, Seq. ID No. 2618, Seq. ID No. 2620, Seq. ID No. 2624, Seq. ID No. 2626, Seq. ID No. 2628, Seq. ID No. 2630, Seq. ID No. 2632, Seq. ID No. 2634, Seq. ID No. 2636, Seq. ID No. 2638, Seq. ID No. 2640, Seq. ID No. 2642, Seq. ID No. 2644, Seq. ID No. 2646, Seq. ID No. 2648, Seq. ID No. 2650, Seq. ID No. 2652, Seq. ID No. 2654, Seq. ID No. 2656, Seq. ID No. 2660, Seq. ID No. 2664, Seq. ID No. 2668, Seq. ID No. 2670, Seq. ID No. 2672, Seq. ID No. 2676, Seq. ID No. 2678, Seq. ID No. 2684, Seq. ID No. 2686, Seq. ID No. 2688, Seq. ID No. 2690, Seq. ID No. 2692, Seq. ID No. 2694, Seq. ID No. 2696, Seq. ID No. 2698, Seq. ID No. 2700, Seq. ID No. 2704, Seq. ID No. 2710, Seq. ID No. 2712, Seq. ID No. 2716, Seq. ID No. 2718, Seq. ID No. 2720, Seq. ID No. 2724, Seq. ID No. 2726, Seq. ID No. 2728, Seq. ID No. 2730, Seq. ID No. 2732, Seq. ID No. 2736, Seq. ID No. 2738, Seq. ID No. 2740, Seq. ID No. 2750, Seq. ID No. 2752, Seq. ID No. 2754, Seq. ID No. 2756, Seq. ID No. 2760, Seq. ID No. 2766, Seq. ID No. 2768, Seq. ID No. 2770, Seq. ID No. 2772, Seq. ID No. 2774, Seq. ID No. 2776, Seq. ID No. 2780, Seq. ID No. 2782, Seq. ID No. 2784, Seq. ID No. 2788, Seq. ID No. 2790, Seq. ID No. 2792, Seq. ID No. 2794, Seq. ID No. 2796, Seq. ID No. 2798, Seq. ID No. 2800, Seq. ID No. 2802, Seq. ID No. 2804, Seq. ID No. 2808, Seq. ID No. 2810, Seq. ID No. 2814, Seq. ID No. 2816, Seq. ID No. 2822, Seq. ID No. 2824, Seq. ID No. 2828, Seq. ID No. 2830, Seq. ID No. 2832, Seq. ID No. 2834, Seq. ID No. 2840, Seq. ID No. 2842, Seq. ID No. 2844, Seq. ID No. 2846, Seq. ID No. 2848, Seq. ID No. 2856, Seq. ID No. 2862, Seq. ID No. 2864, Seq. ID No. 2866, Seq. ID No. 2868, Seq. ID No. 2870, Seq. ID No. 2872, Seq. ID No. 2878, Seq. ID No. 2882, Seq. ID No. 2884, Seq. ID No. 2886, Seq. ID No. 2888, Seq. ID No. 2892, Seq. ID No. 2894, Seq. ID No. 2896, Seq. ID No. 2900, Seq. ID No. 2902, Seq. ID No. 2908, Seq. ID No. 2916, Seq. ID No. 2918, Seq. ID No. 2920, Seq. ID No. 2922, Seq. ID No. 2924, Seq. ID No. 2928, Seq. ID No. 2932, Seq. ID No. 2934, Seq. ID No. 2936, Seq. ID No. 2944, Seq. ID No. 2946, Seq. ID No. 2952, Seq. ID No. 2954, Seq. ID No. 2958, Seq. ID No. 2960, Seq. ID No. 2962, Seq. ID No. 2964, Seq. ID No. 2966, Seq. ID No. 2968, Seq. ID No. 2972, Seq. ID No. 2974, Seq. ID No. 2976, Seq. ID No. 2978, Seq. ID No. 2980, Seq. ID No. 2984, Seq. ID No. 2986, Seq. ID No. 2988, Seq. ID No. 2992, Seq. ID No. 2994, Seq. ID No. 3000, Seq. ID No. 3002, Seq. ID No. 3006, Seq. ID No. 3010, Seq. ID No. 3012, Seq. ID No. 3016, Seq. ID No. 3018, Seq. ID No. 3020, Seq. ID No. 3022, Seq. ID No. 3028, Seq. ID No. 3034, Seq. ID No. 3036, Seq. ID No. 3038, Seq. ID No. 3040, Seq. ID No. 3044, Seq. ID No. 3046, Seq. ID No. 3048, Seq. ID No. 3050, Seq. ID No. 3052, Seq. ID No. 3054, Seq. ID No. 3056, Seq. ID No. 3058, Seq. ID No. 3060, Seq. ID No. 3068, Seq. ID No. 3070, Seq. ID No. 3072, Seq. ID No. 3080, Seq. ID No. 3084, Seq. ID No. 3086, Seq. ID No. 3088, Seq. ID No. 3092, Seq. ID No. 3094, Seq. ID No. 3100, Seq. ID No. 3102, Seq. ID No. 3104, Seq. ID No. 3110, Seq. ID No. 3112, Seq. ID No. 3116, Seq. ID No. 3118, Seq. ID No. 3120, Seq. ID No. 3122, Seq. ID No. 3124, Seq. ID No. 3126, Seq. ID No. 3130, Seq. ID No. 3132, Seq. ID No. 3134, Seq. ID No. 3136, Seq. ID No. 3138, Seq. ID No. 3140, Seq. ID No. 3142, Seq. ID No. 3144, Seq. ID No. 3148, Seq. ID No. 3150, Seq. ID No. 3152, Seq. ID No. 3154, Seq. ID No. 3156, Seq. ID No. 3158, Seq. ID No. 3160, Seq. ID No. 3162, Seq. ID No. 3166, Seq. ID No. 3168, Seq. ID No. 3170, Seq. ID No. 3172, Seq. ID No. 3174, Seq. ID No. 3180, Seq. ID No. 3182, Seq. ID No. 3184, Seq. ID No. 3186, Seq. ID No. 3188, Seq. ID No. 3190, Seq. ID No. 3192, Seq. ID No. 3194, Seq. ID No. 3196, Seq. ID No. 3198, Seq. ID No. 3204, Seq. ID No. 3206, Seq. ID No. 3208, Seq. ID No. 3210, Seq. ID No. 3212, Seq. ID No. 3218, Seq. ID No. 3224, Seq. ID No. 3226, Seq. ID No. 3228, Seq. ID No. 3230, Seq. ID No. 3236, Seq. ID No. 3250, Seq. ID No. 3252, Seq. ID No. 3254, Seq. ID No. 3256, Seq. ID No. 3258, Seq. ID No. 3260, Seq. ID No. 3264, Seq. ID No. 3266, Seq. ID No. 3270, Seq. ID No. 3272, Seq. ID No. 3274, Seq. ID No. 3278, Seq. ID No. 3282, Seq. ID No. 3284, Seq. ID No. 3286, Seq. ID No. 3290, Seq. ID No. 3292, Seq. ID No. 3294, Seq. ID No. 3300, Seq. ID No. 3302, Seq. ID No. 3304, Seq. ID No. 3306, Seq. ID No. 3308, Seq. ID No. 3310, Seq. ID No. 3312, Seq. ID No. 3314, Seq. ID No. 3316, Seq. ID No. 3320, Seq. ID No. 3322, Seq. ID No. 3324, Seq. ID No. 3326, Seq. ID No. 3332, Seq. ID No. 3336, Seq. ID No. 3338, Seq. ID No. 3340, Seq. ID No. 3342, Seq. ID No. 3346, Seq. ID No. 3348, Seq. ID No. 3350, Seq. ID No. 3354, Seq. ID No. 3356, Seq. ID No. 3360, Seq. ID No. 3362, Seq. ID No. 3364, Seq. ID No. 3366, Seq. ID No. 3368, Seq. ID No. 3370, Seq. ID No. 3372, Seq. ID No. 3374, Seq. ID No. 3376, Seq. ID No. 3378, Seq. ID No. 3380, Seq. ID No. 3382, Seq. ID No. 3384, Seq. ID No. 3398, Seq. ID No. 3400, Seq. ID No. 3402, Seq. ID No. 3408, Seq. ID No. 3410, Seq. ID No. 3414, Seq. ID No. 3416, Seq. ID No. 3420, Seq. ID No. 3426, Seq. ID No. 3428, Seq. ID No. 3432, Seq. ID No. 3434, Seq. ID No. 3436, Seq. ID No. 3440, Seq. ID No. 3442, Seq. ID No. 3444, Seq. ID No. 3446, Seq. ID No. 3448, Seq. ID No. 3456, Seq. ID No. 3458, Seq. ID No. 3460, Seq. ID No. 3462, Seq. ID No. 3466, Seq. ID No. 3468, Seq. ID No. 3472, Seq. ID No. 3474, Seq. ID No. 3476, Seq. ID No. 3480, Seq. ID No. 3484, Seq. ID No. 3486, Seq. ID No. 3488, Seq. ID No. 3490, Seq. ID No. 3496, Seq. ID No. 3498, Seq. ID No. 3500, Seq. ID No. 3502, Seq. ID No. 3506, Seq. ID No. 3508, Seq. ID No. 3510, Seq. ID No. 3512, Seq. ID No. 3514, Seq. ID No. 3518, Seq. ID No. 3520, Seq. ID No. 3522, Seq. ID No. 3526, Seq. ID No. 3530, Seq. ID No. 3532, Seq. ID No. 3534, Seq. ID No. 3536, Seq. ID No. 3538, Seq. ID No. 3540, Seq. ID No. 3542, Seq. ID No. 3544, Seq. ID No. 3546, Seq. ID No. 3548, Seq. ID No. 3550, Seq. ID No. 3552, Seq. ID No. 3554, Seq. ID No. 3556, Seq. ID No. 3558, Seq. ID No. 3560, Seq. ID No. 3562, Seq. ID No. 3564, Seq. ID No. 3566, Seq. ID No. 3568, Seq. ID No. 3570, Seq. ID No. 3572, Seq. ID No. 3574, Seq. ID No. 3576, Seq. ID No. 3580, Seq. ID No. 3582, Seq. ID No. 3584, Seq. ID No. 3588, Seq. ID No. 3590, Seq. ID No. 3596, Seq. ID No. 3598, Seq. ID No. 3600, Seq. ID No. 3604, Seq. ID No. 3606, Seq. ID No. 3608, Seq. ID No. 3610, Seq. ID No. 3612, Seq. ID No. 3614, Seq. ID No. 3616, Seq. ID No. 3618, Seq. ID No. 3620, Seq. ID No. 3622, Seq. ID No. 3624, Seq. ID No. 3628, Seq. ID No. 3630, Seq. ID No. 3636, Seq. ID No. 3638, Seq. ID No. 3640, Seq. ID No. 3642, Seq. ID No. 3646, Seq. ID No. 3648, Seq. ID No. 3650, Seq. ID No. 3652, Seq. ID No. 3654, Seq. ID No. 3658, Seq. ID No. 3660, Seq. ID No. 3664, Seq. ID No. 3666, Seq. ID No. 3668, Seq. ID No. 3672, Seq. ID No. 3674, Seq. ID No. 3676, Seq. ID No. 3678, Seq. ID No. 3680, Seq. ID No. 3682, Seq. ID No. 3684, Seq. ID No. 3686, Seq. ID No. 3688, Seq. ID No. 3690, Seq. ID No. 3692, Seq. ID No. 3694, Seq. ID No. 3696, Seq. ID No. 3700, Seq. ID No. 3702, Seq. ID No. 3704, Seq. ID No. 3706, Seq. ID No. 3708, Seq. ID No. 3710, Seq. ID No. 3714, Seq. ID No. 3716, Seq. ID No. 3718, Seq. ID No. 3728, Seq. ID No. 3730, Seq. ID No. 3734, Seq. ID No. 3738, Seq. ID No. 3740, Seq. ID No. 3742, Seq. ID No. 3746, Seq. ID No. 3748, Seq. ID No. 3760, Seq. ID No. 3768, Seq. ID No. 3770, Seq. ID No. 3772, Seq. ID No. 3780, Seq. ID No. 3784, Seq. ID No. 3786, Seq. ID No. 3790, Seq. ID No. 3792, Seq. ID No. 3796, Seq. ID No. 3798, Seq. ID No. 3804, Seq. ID No. 3806, Seq. ID No. 3808, Seq. ID No. 3810, Seq. ID No. 3812, Seq. ID No. 3814, Seq. ID No. 3816, Seq. ID No. 3818, Seq. ID No. 3820, Seq. ID No. 3824, Seq. ID No. 3826, Seq. ID No. 3832, Seq. ID No. 3834, Seq. ID No. 3836, Seq. ID No. 3842, Seq. ID No. 3844, Seq. ID No. 3848, Seq. ID No. 3856, Seq. ID No. 3858, Seq. ID No. 3864, Seq. ID No. 3868, Seq. ID No. 3872, Seq. ID No. 3874, Seq. ID No. 3876, Seq. ID No. 3878, Seq. ID No. 3882, Seq. ID No. 3884, Seq. ID No. 3886, Seq. ID No. 3894, Seq. ID No. 3896, Seq. ID No. 3900, Seq. ID No. 3902, Seq. ID No. 3906, Seq. ID No. 3908, Seq. ID No. 3912, Seq. ID No. 3914, Seq. ID No. 3916, Seq. ID No. 3920, Seq. ID No. 3922, Seq. ID No. 3924, Seq. ID No. 3926, Seq. ID No. 3928, Seq. ID No. 3930, Seq. ID No. 3932, Seq. ID No. 3934, Seq. ID No. 3936, Seq. ID No. 3940, Seq. ID No. 3942, Seq. ID No. 3944, Seq. ID No. 3946, Seq. ID No. 3948, Seq. ID No. 3950, Seq. ID No. 3952, Seq. ID No. 3954, Seq. ID No. 3956, Seq. ID No. 3958, Seq. ID No. 3960, Seq. ID No. 3962, Seq. ID No. 3966, Seq. ID No. 3968, Seq. ID No. 3970, Seq. ID No. 3972, Seq. ID No. 3976, Seq. ID No. 3980, Seq. ID No. 3982, Seq. ID No. 3984, Seq. ID No. 3986, Seq. ID No. 3988, Seq. ID No. 3992, Seq. ID No. 3996, Seq. ID No. 3998, Seq. ID No. 4000, Seq. ID No. 4004, Seq. ID No. 4008, Seq. ID No. 4010, Seq. ID No. 4016, Seq. ID No. 4018, Seq. ID No. 4022, Seq. ID No. 4024, Seq. ID No. 4030, Seq. ID No. 4036, Seq. ID No. 4038, Seq. ID No. 4040, Seq. ID No. 4050, Seq. ID No. 4052, Seq. ID No. 4054, Seq. ID No. 4056, Seq. ID No. 4058, Seq. ID No. 4062, Seq. ID No. 4076, Seq. ID No. 4080, Seq. ID No. 4086, Seq. ID No. 4088, Seq. ID No. 4090, Seq. ID No. 4092, Seq. ID No. 4094, Seq. ID No. 4096, Seq. ID No. 4100, Seq. ID No. 4106, Seq. ID No. 4108, Seq. ID No. 4110, Seq. ID No. 4112, Seq. ID No. 4114, Seq. ID No. 4118, Seq. ID No. 4120, Seq. ID No. 4122, Seq. ID No. 4124, Seq. ID No. 4126, Seq. ID No. 4128, Seq. ID No. 4130, Seq. ID No. 4132, Seq. ID No. 4134, Seq. ID No. 4136, Seq. ID No. 4138, Seq. ID No. 4140, Seq. ID No. 4142, Seq. ID No. 4144, Seq. ID No. 4146, Seq. ID No. 4150, Seq. ID No. 4154, Seq. ID No. 4156, Seq. ID No. 4158, Seq. ID No. 4162, Seq. ID No. 4164, Seq. ID No. 4168, Seq. ID No. 4172, Seq. ID No. 4174, Seq. ID No. 4176, Seq. ID No. 4178, Seq. ID No. 4180, Seq. ID No. 4182, Seq. ID No. 4186, Seq. ID No. 4188, Seq. ID No. 4190, Seq. ID No. 4192, Seq. ID No. 4194, Seq. ID No. 4196, Seq. ID No. 4198, Seq. ID No. 4200, Seq. ID No. 4204, Seq. ID No. 4206, Seq. ID No. 4208, Seq. ID No. 4210, Seq. ID No. 4212, Seq. ID No. 4220, Seq. ID No. 4230, Seq. ID No. 4234, Seq. ID No. 4236, Seq. ID No. 4238, Seq. ID No. 4240, Seq. ID No. 4244, Seq. ID No. 4250, Seq. ID No. 4252, Seq. ID No. 4254, Seq. ID No. 4256, Seq. ID No. 4260, Seq. ID No. 4264, Seq. ID No. 4266, Seq. ID No. 4268, Seq. ID No. 4270, Seq. ID No. 4272, Seq. ID No. 4274, Seq. ID No. 4276, Seq. ID No. 4282, Seq. ID No. 4284, Seq. ID No. 4286, Seq. ID No. 4288, Seq. ID No. 4290, Seq. ID No. 4292, Seq. ID No. 4294, Seq. ID No. 4296, Seq. ID No. 4298, Seq. ID No. 4300, Seq. ID No. 4302, Seq. ID No. 4304, Seq. ID No. 4308, Seq. ID No. 4310, Seq. ID No. 4314, Seq. ID No. 4316, Seq. ID No. 4318, Seq. ID No. 4320, Seq. ID No. 4322, Seq. ID No. 4324, Seq. ID No. 4326, Seq. ID No. 4330, Seq. ID No. 4332, Seq. ID No. 4334, Seq. ID No. 4336, Seq. ID No. 4338, Seq. ID No. 4340, Seq. ID No. 4344, Seq. ID No. 4346, Seq. ID No. 4348, Seq. ID No. 4352, Seq. ID No. 4354, Seq. ID No. 4356, Seq. ID No. 4360, Seq. ID No. 4362, Seq. ID No. 4364, Seq. ID No. 4366, Seq. ID No. 4368, Seq. ID No. 4370, Seq. ID No. 4376, Seq. ID No. 4378, Seq. ID No. 4380, Seq. ID No. 4384, Seq. ID No. 4390, Seq. ID No. 4392, Seq. ID No. 4394, Seq. ID No. 4396, Seq. ID No. 4398, Seq. ID No. 4400, Seq. ID No. 4402, Seq. ID No. 4404, Seq. ID No. 4406, Seq. ID No. 4408, Seq. ID No. 4410, Seq. ID No. 4412, Seq. ID No. 4414, Seq. ID No. 4416, Seq. ID No. 4418, Seq. ID No. 4420, Seq. ID No. 4422, Seq. ID No. 4424, Seq. ID No. 4426, Seq. ID No. 4428, Seq. ID No. 4430, Seq. ID No. 4432, Seq. ID No. 4436, Seq. ID No. 4438, Seq. ID No. 4440, Seq. ID No. 4442, Seq. ID No. 4450, Seq. ID No. 4452, Seq. ID No. 4454, Seq. ID No. 4456, Seq. ID No. 4458, Seq. ID No. 4460, Seq. ID No. 4462, Seq. ID No. 4466, Seq. ID No. 4468, Seq. ID No. 4472, Seq. ID No. 4476, Seq. ID No. 4478, Seq. ID No. 4480, Seq. ID No. 4482, Seq. ID No. 4486, Seq. ID No. 4488, Seq. ID No. 4490, Seq. ID No. 4492, Seq. ID No. 4494, Seq. ID No. 4496, Seq. ID No. 4498, Seq. ID No. 4502, Seq. ID No. 4504, Seq. ID No. 4506, Seq. ID No. 4508, Seq. ID No. 4510, Seq. ID No. 4512, Seq. ID No. 4514, Seq. ID No. 4516, Seq. ID No. 4520, Seq. ID No. 4522, Seq. ID No. 4524, Seq. ID No. 4526, Seq. ID No. 4528, Seq. ID No. 4530, Seq. ID No. 4532, Seq. ID No. 4534, Seq. ID No. 4538, Seq. ID No. 4542, Seq. ID No. 4546, Seq. ID No. 4548, Seq. ID No. 4550, Seq. ID No. 4552, Seq. ID No. 4554, Seq. ID No. 4556, Seq. ID No. 4560, Seq. ID No. 4562, Seq. ID No. 4566, Seq. ID No. 4568, Seq. ID No. 4572, Seq. ID No. 4574, Seq. ID No. 4576, Seq. ID No. 4578, Seq. ID No. 4582, Seq. ID No. 4586, Seq. ID No. 4590, Seq. ID No. 4592, Seq. ID No. 4598, Seq. ID No. 4600, Seq. ID No. 4602, Seq. ID No. 4604, Seq. ID No. 4606, Seq. ID No. 4608, Seq. ID No. 4610, Seq. ID No. 4612, Seq. ID No. 4614, Seq. ID No. 4616, Seq. ID No. 4618, Seq. ID No. 4620, Seq. ID No. 4622, Seq. ID No. 4624, Seq. ID No. 4628, Seq. ID No. 4632, Seq. ID No. 4638, Seq. ID No. 4642, Seq. ID No. 4644, Seq. ID No. 4646, Seq. ID No. 4648, Seq. ID No. 4650, Seq. ID No. 4654, Seq. ID No. 4660, Seq. ID No. 4662, Seq. ID No. 4664, Seq. ID No. 4666, Seq. ID No. 4668, Seq. ID No. 4670, Seq. ID No. 4672, Seq. ID No. 4674, Seq. ID No. 4676, Seq. ID No. 4678, Seq. ID No. 4680, Seq. ID No. 4684, Seq. ID No. 4686, Seq. ID No. 4688, Seq. ID No. 4692, Seq. ID No. 4694, Seq. ID No. 4696, Seq. ID No. 4700, Seq. ID No. 4702, Seq. ID No. 4704, Seq. ID No. 4706, Seq. ID No. 4714, Seq. ID No. 4718, Seq. ID No. 4720, Seq. ID No. 4722, Seq. ID No. 4728, Seq. ID No. 4730, Seq. ID No. 4734, Seq. ID No. 4736, Seq. ID No. 4738, Seq. ID No. 4740, Seq. ID No. 4742, Seq. ID No. 4744, Seq. ID No. 4748, Seq. ID No. 4750, Seq. ID No. 4752, Seq. ID No. 4756, Seq. ID No. 4758, Seq. ID No. 4764, Seq. ID No. 4766, Seq. ID No. 4768, Seq. ID No. 4770, Seq. ID No. 4774, Seq. ID No. 4776, Seq. ID No. 4778, Seq. ID No. 4780, Seq. ID No. 4782, Seq. ID No. 4788, Seq. ID No. 4790, Seq. ID No. 4792, Seq. ID No. 4794, Seq. ID No. 4796, Seq. ID No. 4798, Seq. ID No. 4802, Seq. ID No. 4804, Seq. ID No. 4806, Seq. ID No. 4810, Seq. ID No. 4812, Seq. ID No. 4814, Seq. ID No. 4816, Seq. ID No. 4818, Seq. ID No. 4820, Seq. ID No. 4826, Seq. ID No. 4828, Seq. ID No. 4830, Seq. ID No. 4832, Seq. ID No. 4834, Seq. ID No. 4838, Seq. ID No. 4840, Seq. ID No. 4842, Seq. ID No. 4844, Seq. ID No. 4846, Seq. ID No. 4850, Seq. ID No. 4852, Seq. ID No. 4854, Seq. ID No. 4856, Seq. ID No. 4862, Seq. ID No. 4864, Seq. ID No. 4868, Seq. ID No. 4876, Seq. ID No. 4878, Seq. ID No. 4880, Seq. ID No. 4882, Seq. ID No. 4884, Seq. ID No. 4890, Seq. ID No. 4892, Seq. ID No. 4894, Seq. ID No. 4896, Seq. ID No. 4898, Seq. ID No. 4900, Seq. ID No. 4902, Seq. ID No. 4904, Seq. ID No. 4906, Seq. ID No. 4908, Seq. ID No. 4910, Seq. ID No. 4912, Seq. ID No. 4914, Seq. ID No. 4916, Seq. ID No. 4918, Seq. ID No. 4920, Seq. ID No. 4922, Seq. ID No. 4924, Seq. ID No. 4928, Seq. ID No. 4932, Seq. ID No. 4936, Seq. ID No. 4938, Seq. ID No. 4940, Seq. ID No. 4942, Seq. ID No. 4944, Seq. ID No. 4946, Seq. ID No. 4948, Seq. ID No. 4950, Seq. ID No. 4952, Seq. ID No. 4954, Seq. ID No. 4958, Seq. ID No. 4960, Seq. ID No. 4962, Seq. ID No. 4964, Seq. ID No. 4974, Seq. ID No. 4976, Seq. ID No. 4978, Seq. ID No. 4980, Seq. ID No. 4982, Seq. ID No. 4984, Seq. ID No. 4986, Seq. ID No. 4988, Seq. ID No. 4990, Seq. ID No. 4992, Seq. ID No. 4994, Seq. ID No. 4996, Seq. ID No. 4998, Seq. ID No. 5000, Seq. ID No. 5004, Seq. ID No. 5006, Seq. ID No. 5008, Seq. ID No. 5010, Seq. ID No. 5012, Seq. ID No. 5014, Seq. ID No. 5018, Seq. ID No. 5020, Seq. ID No. 5022, Seq. ID No. 5024, Seq. ID No. 5026, Seq. ID No. 5030, Seq. ID No. 5032, Seq. ID No. 5034, Seq. ID No. 5036, Seq. ID No. 5042, Seq. ID No. 5044, Seq. ID No. 5046, Seq. ID No. 5048, Seq. ID No. 5058, Seq. ID No. 5060, Seq. ID No. 5064, Seq. ID No. 5066, Seq. ID No. 5068, Seq. ID No. 5070, Seq. ID No. 5076, Seq. ID No. 5078, Seq. ID No. 5080, Seq. ID No. 5082, Seq. ID No. 5084, Seq. ID No. 5086, Seq. ID No. 5088, Seq. ID No. 5092, Seq. ID No. 5100, Seq. ID No. 5102, Seq. ID No. 5104, Seq. ID No. 5110, Seq. ID No. 5112, Seq. ID No. 5114, Seq. ID No. 5116, Seq. ID No. 5118, Seq. ID No. 5122, Seq. ID No. 5126, Seq. ID No. 5128, Seq. ID No. 5134, Seq. ID No. 5140, Seq. ID No. 5142, Seq. ID No. 5146, Seq. ID No. 5148, Seq. ID No. 5150, Seq. ID No. 5152, Seq. ID No. 5156, Seq. ID No. 5158, Seq. ID No. 5160, Seq. ID No. 5162, Seq. ID No. 5166, Seq. ID No. 5168, Seq. ID No. 5170, Seq. ID No. 5178, Seq. ID No. 5182, Seq. ID No. 5184, Seq. ID No. 5186, Seq. ID No. 5188, Seq. ID No. 5190, Seq. ID No. 5192, Seq. ID No. 5194, Seq. ID No. 5196, Seq. ID No. 5198, Seq. ID No. 5200, Seq. ID No. 5202, Seq. ID No. 5204, Seq. ID No. 5206, Seq. ID No. 5210, Seq. ID No. 5212, Seq. ID No. 5216, Seq. ID No. 5220, Seq. ID No. 5222, Seq. ID No. 5230, Seq. ID No. 5232, Seq. ID No. 5240, Seq. ID No. 5242, Seq. ID No. 5244, Seq. ID No. 5246, Seq. ID No. 5252, Seq. ID No. 5254, Seq. ID No. 5256, Seq. ID No. 5262, Seq. ID No. 5264, Seq. ID No. 5266, Seq. ID No. 5268, Seq. ID No. 5278, Seq. ID No. 5280, Seq. ID No. 5282, Seq. ID No. 5286, Seq. ID No. 5288, Seq. ID No. 5292, Seq. ID No. 5296, Seq. ID No. 5298, Seq. ID No. 5300, Seq. ID No. 5302, Seq. ID No. 5304, Seq. ID No. 5306, Seq. ID No. 5308, Seq. ID No. 5310, Seq. ID No. 5312, Seq. ID No. 5314, Seq. ID No. 5318, Seq. ID No. 5320, Seq. ID No. 5322, Seq. ID No. 5324, Seq. ID No. 5328, Seq. ID No. 5330, Seq. ID No. 5332, Seq. ID No. 5334, Seq. ID No. 5336, Seq. ID No. 5338, Seq. ID No. 5344, Seq. ID No. 5346, Seq. ID No. 5350, Seq. ID No. 5352, Seq. ID No. 5358, Seq. ID No. 5360, Seq. ID No. 5366, Seq. ID No. 5368, Seq. ID No. 5374, Seq. ID No. 5378, Seq. ID No. 5380, Seq. ID No. 5382, Seq. ID No. 5384, Seq. ID No. 5388, Seq. ID No. 5390, Seq. ID No. 5392, Seq. ID No. 5394, Seq. ID No. 5398, Seq. ID No. 5400, Seq. ID No. 5402, Seq. ID No. 5404, Seq. ID No. 5406, Seq. ID No. 5408, Seq. ID No. 5410, Seq. ID No. 5412, Seq. ID No. 5416, Seq. ID No. 5418, Seq. ID No. 5420, Seq. ID No. 5422, Seq. ID No. 5424, Seq. ID No. 5426, Seq. ID No. 5428, Seq. ID No. 5432, Seq. ID No. 5434, Seq. ID No. 5436, Seq. ID No. 5438, Seq. ID No. 5440, Seq. ID No. 5442, Seq. ID No. 5444, Seq. ID No. 5448, Seq. ID No. 5450, Seq. ID No. 5452, Seq. ID No. 5454, Seq. ID No. 5456, Seq. ID No. 5458, Seq. ID No. 5468, Seq. ID No. 5472, Seq. ID No. 5474, Seq. ID No. 5478, Seq. ID No. 5480, Seq. ID No. 5482, Seq. ID No. 5492, Seq. ID No. 5494, Seq. ID No. 5496, Seq. ID No. 5500, Seq. ID No. 5504, Seq. ID No. 5510, Seq. ID No. 5514, Seq. ID No. 5516, Seq. ID No. 5518, Seq. ID No. 5520, Seq. ID No. 5522, Seq. ID No. 5528, Seq. ID No. 5530, Seq. ID No. 5532, Seq. ID No. 5534, Seq. ID No. 5538, Seq. ID No. 5540, Seq. ID No. 5542, Seq. ID No. 5544, Seq. ID No. 5546, Seq. ID No. 5548, Seq. ID No. 5550, Seq. ID No. 5552, Seq. ID No. 5554, Seq. ID No. 5558, Seq. ID No. 5560, Seq. ID No. 5564, Seq. ID No. 5568, Seq. ID No. 5570, Seq. ID No. 5576, Seq. ID No. 5578, Seq. ID No. 5582, Seq. ID No. 5584, Seq. ID No. 5586, Seq. ID No. 5590, Seq. ID No. 5592, Seq. ID No. 5594, Seq. ID No. 5596, Seq. ID No. 5600, Seq. ID No. 5602, Seq. ID No. 5608, Seq. ID No. 5614, Seq. ID No. 5616, Seq. ID No. 5618, Seq. ID No. 5620, Seq. ID No. 5622, Seq. ID No. 5626, Seq. ID No. 5628, Seq. ID No. 5630, Seq. ID No. 5632, Seq. ID No. 5638, Seq. ID No. 5640, Seq. ID No. 5642, Seq. ID No. 5646, Seq. ID No. 5648, Seq. ID No. 5650, Seq. ID No. 5652, Seq. ID No. 5656, Seq. ID No. 5658, Seq. ID No. 5662, Seq. ID No. 5666, Seq. ID No. 5668, Seq. ID No. 5670, Seq. ID No. 5678, Seq. ID No. 5680, Seq. ID No. 5684, Seq. ID No. 5686, Seq. ID No. 5688, Seq. ID No. 5690, Seq. ID No. 5692, Seq. ID No. 5694, Seq. ID No. 5698, Seq. ID No. 5700, Seq. ID No. 5702, Seq. ID No. 5704, Seq. ID No. 5706, Seq. ID No. 5708, Seq. ID No. 5712, Seq. ID No. 5714, Seq. ID No. 5716, Seq. ID No. 5718, Seq. ID No. 5720, Seq. ID No. 5722, Seq. ID No. 5724, Seq. ID No. 5726, Seq. ID No. 5728, Seq. ID No. 5736, Seq. ID No. 5740, Seq. ID No. 5742, Seq. ID No. 5744, Seq. ID No. 5746, Seq. ID No. 5748, Seq. ID No. 5750, Seq. ID No. 5752, Seq. ID No. 5754, Seq. ID No. 5756, Seq. ID No. 5758, Seq. ID No. 5760, Seq. ID No. 5762, Seq. ID No. 5770, Seq. ID No. 5772, Seq. ID No. 5778, Seq. ID No. 5780, Seq. ID No. 5784, Seq. ID No. 5788, Seq. ID No. 5792, Seq. ID No. 5796, Seq. ID No. 5800, Seq. ID No. 5802, Seq. ID No. 5808, Seq. ID No. 5810, Seq. ID No. 5814, Seq. ID No. 5816, Seq. ID No. 5820, Seq. ID No. 5822, Seq. ID No. 5824, Seq. ID No. 5826, Seq. ID No. 5828, Seq. ID No. 5830, Seq. ID No. 5832, Seq. ID No. 5834, Seq. ID No. 5836, Seq. ID No. 5838, Seq. ID No. 5840, Seq. ID No. 5842, Seq. ID No. 5846, Seq. ID No. 5848, Seq. ID No. 5850, Seq. ID No. 5852, Seq. ID No. 5856, Seq. ID No. 5858, Seq. ID No. 5860, Seq. ID No. 5862, Seq. ID No. 5864, Seq. ID No. 5866, Seq. ID No. 5870, Seq. ID No. 5872, Seq. ID No. 5874, Seq. ID No. 5876, Seq. ID No. 5880, Seq. ID No. 5882, Seq. ID No. 5884, Seq. ID No. 5890, Seq. ID No. 5896, Seq. ID No. 5898, Seq. ID No. 5902, Seq. ID No. 5904, Seq. ID No. 5912, Seq. ID No. 5914, Seq. ID No. 5916, Seq. ID No. 5918, Seq. ID No. 5920, Seq. ID No. 5928, Seq. ID No. 5930, Seq. ID No. 5934, Seq. ID No. 5936, Seq. ID No. 5938, Seq. ID No. 5942, Seq. ID No. 5946, Seq. ID No. 5948, Seq. ID No. 5952, Seq. ID No. 5960, Seq. ID No. 5962, Seq. ID No. 5964, Seq. ID No. 5966, Seq. ID No. 5968, Seq. ID No. 5970, Seq. ID No. 5972, Seq. ID No. 5974, Seq. ID No. 5978, Seq. ID No. 5980, Seq. ID No. 5982, Seq. ID No. 5986, Seq. ID No. 5988, Seq. ID No. 5990, Seq. ID No. 5992, Seq. ID No. 5994, Seq. ID No. 5996, Seq. ID No. 6000, Seq. ID No. 6002, Seq. ID No. 6004, Seq. ID No. 6010, Seq. ID No. 6012, Seq. ID No. 6014, Seq. ID No. 6016, Seq. ID No. 6018, Seq. ID No. 6026, Seq. ID No. 6028, Seq. ID No. 6030, Seq. ID No. 6032, Seq. ID No. 6034, Seq. ID No. 6036, Seq. ID No. 6040, Seq. ID No. 6042, Seq. ID No. 6044, Seq. ID No. 6046, Seq. ID No. 6050, Seq. ID No. 6052, Seq. ID No. 6054, Seq. ID No. 6056, Seq. ID No. 6058, Seq. ID No. 6062, Seq. ID No. 6064, Seq. ID No. 6066, Seq. ID No. 6070, Seq. ID No. 6072, Seq. ID No. 6074, Seq. ID No. 6078, Seq. ID No. 6080, Seq. ID No. 6086, Seq. ID No. 6088, Seq. ID No. 6090, Seq. ID No. 6094, Seq. ID No. 6096, Seq. ID No. 6102, Seq. ID No. 6104, Seq. ID No. 6110, Seq. ID No. 6112, Seq. ID No. 6118, Seq. ID No. 6122, Seq. ID No. 6124, Seq. ID No. 6130, Seq. ID No. 6132, Seq. ID No. 6134, Seq. ID No. 6136, Seq. ID No. 6138, Seq. ID No. 6140, Seq. ID No. 6142, Seq. ID No. 6146, Seq. ID No. 6152, Seq. ID No. 6156, Seq. ID No. 6158, Seq. ID No. 6160, Seq. ID No. 6166, Seq. ID No. 6168, Seq. ID No. 6170, Seq. ID No. 6172, Seq. ID No. 6176, Seq. ID No. 6178, Seq. ID No. 6182, Seq. ID No. 6188, Seq. ID No. 6194, Seq. ID No. 6196, Seq. ID No. 6198, Seq. ID No. 6200, Seq. ID No. 6202, Seq. ID No. 6210, Seq. ID No. 6214, Seq. ID No. 6216, Seq. ID No. 6218, Seq. ID No. 6220, Seq. ID No. 6232, Seq. ID No. 6234, Seq. ID No. 6236, Seq. ID No. 6240, Seq. ID No. 6242, Seq. ID No. 6244, Seq. ID No. 6250, Seq. ID No. 6258, Seq. ID No. 6260, Seq. ID No. 6262, Seq. ID No. 6264, Seq. ID No. 6268, Seq. ID No. 6270, Seq. ID No. 6272, Seq. ID No. 6276, Seq. ID No. 6278, Seq. ID No. 6280, Seq. ID No. 6282, Seq. ID No. 6284, Seq. ID No. 6286, Seq. ID No. 6288, Seq. ID No. 6294, Seq. ID No: 6296, Seq. ID No. 6298, Seq. ID No. 6300, Seq. ID No. 6306, Seq. ID No. 6310, Seq. ID No. 6312, Seq. ID No. 6316, Seq. ID No. 6318, Seq. ID No. 6320, Seq. ID No. 6322, Seq. ID No. 6324, Seq. ID No. 6326, Seq. ID No. 6328, Seq. ID No. 6330, Seq. ID No. 6332, Seq. ID No. 6336, Seq. ID No. 6338, Seq. ID No. 6342, Seq. ID No. 6346, Seq. ID No. 6350, Seq. ID No. 6352, Seq. ID No. 6354, Seq. ID No. 6360, Seq. ID No. 6362, Seq. ID No. 6368, Seq. ID No. 6370, Seq. ID No. 6372, Seq. ID No. 6374, Seq. ID No. 6376, Seq. ID No. 6378, Seq. ID No. 6380, Seq. ID No. 6382, Seq. ID No. 6386, Seq. ID No. 6390, Seq. ID No. 6394, Seq. ID No. 6396, Seq. ID No. 6400, Seq. ID No. 6410, Seq. ID No. 6412, Seq. ID No. 6414, Seq. ID No. 6418, Seq. ID No. 6422, Seq. ID No. 6424, Seq. ID No. 6426, Seq. ID No. 6428, Seq. ID No. 6430, Seq. ID No. 6432, Seq. ID No. 6442, Seq. ID No. 644.4, Seq. ID No. 6446, Seq. ID No. 6452, Seq. ID No. 6454, Seq. ID No. 6460, Seq. ID No. 6462, Seq. ID No. 6464, Seq. ID No. 6466, Seq. ID No. 6468, Seq. ID No. 6470, Seq. ID No. 6472, Seq. ID No. 6474, Seq. ID No. 6480, Seq. ID No. 6484, Seq. ID No. 6494, Seq. ID No. 6496, Seq. ID No. 6498, Seq. ID No. 6502, Seq. ID No. 6504, Seq. ID No. 6506, Seq. ID No. 6508, Seq. ID No. 6512, Seq. ID No. 6518, Seq. ID No. 6524, Seq. ID No. 6526, Seq. ID No. 6536, Seq. ID No. 6538, Seq. ID No. 6540, Seq. ID No. 6542, Seq. ID No. 6544, Seq. ID No. 6546, Seq. ID No. 6548, Seq. ID No. 6550, Seq. ID No. 6552, Seq. ID No. 6554, Seq. ID No. 6556, Seq. ID No. 6558, Seq. ID No. 6560, Seq. ID No. 6564, Seq. ID No. 6574, Seq. ID No. 6576, Seq. ID No. 6578, Seq. ID No. 6580, Seq. ID No. 6584, Seq. ID No. 6586, Seq. ID No. 6588, Seq. ID No. 6592, Seq. ID No. 6598, Seq. ID No. 6602, Seq. ID No. 6604, Seq. ID No. 6606, Seq. ID No. 6610, Seq. ID No. 6612, Seq. ID No. 6614, Seq. ID No. 6616, Seq. ID No. 6618, Seq. ID No. 6620, Seq. ID No. 6622, Seq. ID No. 6624, Seq. ID No. 6628, Seq. ID No. 6630, Seq. ID No. 6632, Seq. ID No. 6634, Seq. ID No. 6638, Seq. ID No. 6640, Seq. ID No. 6644, Seq. ID No. 6646, and Seq. ID No. 6650.

55. The Alloiococcus otitidis polypeptide of claim 36, wherein the polypeptide is identified by an ORF finder program that searches for a transmembrane domain between two Stop codons and a Start codon immediately upstream of this transmembrane region, and is selected from one of Seq. ID No. 8, Seq. ID No. 10, Seq. ID No. 34, Seq. ID No. 48, Seq. ID No. 68, Seq. ID No. 86, Seq. ID No. 88, Seq. ID No. 124, Seq. ID No. 134, Seq. ID No. 142, Seq. ID No. 160, Seq. ID No. 164, Seq. ID No. 192, Seq. ID No. 210, Seq. ID No. 212, Seq. ID No. 216, Seq. ID No. 218, Seq. ID No. 226, Seq. ID No. 236, Seq. ID No. 238, Seq. ID No. 250, Seq. ID No. 252, Seq. ID No. 256, Seq. ID No. 272, Seq. ID No. 278, Seq. ID No. 286, Seq. ID No. 288, Seq. ID No. 290, Seq. ID No. 328, Seq. ID No. 330, Seq. ID No. 374, Seq. ID No. 376, Seq. ID No. 420, Seq. ID No. 488, Seq. ID No. 498, Seq. ID No. 500, Seq. ID No. 504, Seq. ID No. 506, Seq. ID No. 510, Seq. ID No. 512, Seq. ID No. 516, Seq. ID No. 526, Seq. ID No. 528, Seq. ID No. 546, Seq. ID No. 568, Seq. ID No. 570, Seq. ID No. 576, Seq. ID No. 578, Seq. ID No. 580, Seq. ID No. 594, Seq. ID No. 618, Seq. ID No. 638, Seq. ID No. 640, Seq. ID No. 642, Seq. ID No. 646, Seq. ID No. 654, Seq. ID No. 656, Seq. ID No. 660, Seq. ID No. 670, Seq. ID No. 676, Seq. ID No. 678, Seq. ID No. 680, Seq. ID No. 716, Seq. ID No. 718, Seq. ID No. 720, Seq. ID No. 722, Seq. ID No. 724, Seq. ID No. 748, Seq. ID No. 752, Seq. ID No. 756, Seq. ID No. 758, Seq. ID No. 760, Seq. ID No. 762, Seq. ID No. 764, Seq. ID No. 770, Seq. ID No. 778, Seq. ID No. 780, Seq. ID No. 808, Seq. ID No. 810, Seq. ID No. 820, Seq. ID No. 826, Seq. ID No. 864, Seq. ID No. 894, Seq. ID No. 902, Seq. ID No. 936, Seq. ID No. 944, Seq. ID No. 956, Seq. ID No. 968, Seq. ID No. 980, Seq. ID No. 1006, Seq. ID No. 1010, Seq. ID No. 1012, Seq. ID No. 1014, Seq. ID No. 1016, Seq. ID No. 1020, Seq. ID No. 1030, Seq. ID No. 1032, Seq. ID No. 1034, Seq. ID No. 1038, Seq. ID No. 1040, Seq. ID No. 1046, Seq. ID No. 1056, Seq. ID No. 1076, Seq. ID No. 1106, Seq. ID No. 1132, Seq. ID No. 1136, Seq. ID No. 1138, Seq. ID No. 1166, Seq. ID No. 1168, Seq. ID No. 1170, Seq. ID No. 1184, Seq. ID No. 1194, Seq. ID No. 1198, Seq. ID No. 1214, Seq. ID No. 1224, Seq. ID No. 1228, Seq. ID No. 1230, Seq. ID No. 1238, Seq. ID No. 1244, Seq. ID No. 1266, Seq. ID No. 1268, Seq. ID No. 1270, Seq. ID No. 1272, Seq. ID No. 1284, Seq. ID No. 1288, Seq. ID No. 1292, Seq. ID No. 1298, Seq. ID No. 1306, Seq. ID No. 1316, Seq. ID No. 1326, Seq. ID No. 1328, Seq. ID No. 1330, Seq. ID No. 1332, Seq. ID No. 1342, Seq. ID No. 1354, Seq. ID No. 1368, Seq. ID No. 1402, Seq. ID No. 1430, Seq. ID No. 1436, Seq. ID No. 1446, Seq. ID No. 1482, Seq. ID No. 1514, Seq. ID No. 1516, Seq. ID No. 1518, Seq. ID No. 1530, Seq. ID No. 1534, Seq. ID No. 1536, Seq. ID No. 1546, Seq. ID No. 1548, Seq. ID No. 1552, Seq. ID No. 1560, Seq. ID No. 1564, Seq. ID No. 1572, Seq. ID No. 1580, Seq. ID No. 1584, Seq. ID No. 1586, Seq. ID No. 1622, Seq. ID No. 1634, Seq. ID No. 1644, Seq. ID No. 1646, Seq. ID No. 1648, Seq. ID No. 1650, Seq. ID No. 1654, Seq. ID No. 1672, Seq. ID No. 1674, Seq. ID No. 1678, Seq. ID No. 1682, Seq. ID No. 1708, Seq. ID No. 1732, Seq. ID No. 1734, Seq. ID No. 1738; Seq. ID No. 1754, Seq: ID No. 1758, Seq. ID No. 1766, Seq. ID No. 1768, Seq. ID No. 1788, Seq. ID No. 1792, Seq. ID No. 1798, Seq. ID No. 1806, Seq. ID No. 1820, Seq. ID No. 1822, Seq. ID No. 1824, Seq. ID No. 1840, Seq. ID No. 1870, Seq. ID No. 1876, Seq. ID No. 1892, Seq. ID No. 1894, Seq. ID No. 1896, Seq. ID No. 1924, Seq. ID No. 1928, Seq. ID No. 1970, Seq. ID No. 1982, Seq. ID No. 1992, Seq. ID No. 1994, Seq. ID No. 2014, Seq. ID No. 2018, Seq. ID No. 2028, Seq. ID No. 2034, Seq. ID No. 2042, Seq. ID No. 2050, Seq. ID No. 2054, Seq. ID No. 2088, Seq. ID No. 2090, Seq. ID No. 2106, Seq. ID No. 2116, Seq. ID No. 2122, Seq. ID No. 2128, Seq. ID No. 2152, Seq. ID No. 2160, Seq. ID No. 2172, Seq. ID No. 2174, Seq. ID No. 2178, Seq. ID No. 2188, Seq. ID No. 2238, Seq. ID No. 2240, Seq. ID No. 2246, Seq. ID No. 2250, Seq. ID No. 2272, Seq. ID No. 2284, Seq. ID No. 2290, Seq. ID No. 2292, Seq. ID No. 2294, Seq. ID No. 2296, Seq. ID No. 2304, Seq. ID No. 2306, Seq. ID No. 2308, Seq. ID No. 2318, Seq. ID No. 2320, Seq. ID No. 2322, Seq. ID No. 2324, Seq. ID No. 2326, Seq. ID No. 2328, Seq. ID No. 2334, Seq. ID No. 2344, Seq. ID No. 2348, Seq. ID No. 2350, Seq. ID No. 2352, Seq. ID No. 2354, Seq. ID No. 2356, Seq. ID No. 2358, Seq. ID No. 2366, Seq. ID No. 2390, Seq. ID No. 2392, Seq. ID No. 2408, Seq. ID No. 2420, Seq. ID No. 2422, Seq. ID No. 2428, Seq. ID No. 2430, Seq. ID No. 2434, Seq. ID No. 2450, Seq. ID No. 2460, Seq. ID No. 2490, Seq. ID No. 2498, Seq. ID No. 2504, Seq. ID No. 2508, Seq. ID No. 2516, Seq. ID No. 2524, Seq. ID No. 2526, Seq. ID No. 2532, Seq. ID No. 2536, Seq. ID No. 2538, Seq. ID No. 2542, Seq. ID No. 2544, Seq. ID No. 2546, Seq. ID No. 2556, Seq. ID No. 2558, Seq. ID No. 2570, Seq. ID No. 2572, Seq. ID No. 2584, Seq. ID No. 2592, Seq. ID No. 2598, Seq. ID No. 2600, Seq. ID No. 2602, Seq. ID No. 2608, Seq. ID No. 2612, Seq. ID No. 2622, Seq. ID No. 2630, Seq. ID No. 2652, Seq. ID No. 2680, Seq. ID No. 2694, Seq. ID No. 2702, Seq. ID No. 2704, Seq. ID No. 2706, Seq. ID No. 2708, Seq. ID No. 2732, Seq. ID No. 2734, Seq. ID No. 2784, Seq. ID No. 2786, Seq. ID No. 2814, Seq. ID No. 2816, Seq. ID No. 2824, Seq. ID No. 2836, Seq. ID No. 2838, Seq. ID No. 2842, Seq. ID No. 2854, Seq. ID No. 2874, Seq. ID No. 2876, Seq. ID No. 2882, Seq. ID No. 2906, Seq. ID No. 2936, Seq. ID No. 2938, Seq. ID No. 2940, Seq. ID No. 2942, Seq. ID No. 2948, Seq. ID No. 2950, Seq. ID No. 2970, Seq. ID No. 2974, Seq. ID No. 2978, Seq. ID No. 2982, Seq. ID No. 2988, Seq. ID No. 2992, Seq. ID No. 2996, Seq. ID No. 2998, Seq. ID No. 3006, Seq. ID No. 3008, Seq. ID No. 3014, Seq. ID No. 3024, Seq. ID No. 3026, Seq. ID No. 3028, Seq. ID No. 3030, Seq. ID No. 3032, Seq. ID No. 3044, Seq. ID No. 3056, Seq. ID No. 3064, Seq. ID No. 3072, Seq. ID No. 3074, Seq. ID No. 3076, Seq. ID No. 3078, Seq. ID No. 3124, Seq. ID No. 3146, Seq. ID No. 3156, Seq. ID No. 3158, Seq. ID No. 3164, Seq. ID No. 3184, Seq. ID No. 3194, Seq. ID No. 3202, Seq. ID No. 3214, Seq. ID No. 3216, Seq. ID No. 3218, Seq. ID No. 3220, Seq. ID No. 3242, Seq. ID No. 3244, Seq. ID No. 3246, Seq. ID No. 3250, Seq. ID No. 3258, Seq. ID No. 3262, Seq. ID No. 3270, Seq. ID No. 3276, Seq. ID No. 3298, Seq. ID No. 3318, Seq. ID No. 3324, Seq. ID No. 3338, Seq. ID No. 3340, Seq. ID No. 3350, Seq. ID No. 3352, Seq. ID No. 3354, Seq. ID No. 3358, Seq. ID No. 3360, Seq. ID No. 3378, Seq. ID No. 3390, Seq. ID No. 3392, Seq. ID No. 3396, Seq. ID No. 3406, Seq. ID No. 3422, Seq. ID No. 3426, Seq. ID No. 3450, Seq. ID No. 3452, Seq. ID No. 3454, Seq. ID No. 3456, Seq. ID No. 3458, Seq. ID No. 3464, Seq. ID No. 3482, Seq. ID No. 3492, Seq. ID No. 3494, Seq. ID No. 3504, Seq. ID No. 3506, Seq. ID No. 3528, Seq. ID No. 3594, Seq. ID No. 3596, Seq. ID No. 3622, Seq. ID No. 3626, Seq. ID No. 3636, Seq. ID No. 3680, Seq. ID No. 3724, Seq. ID No. 3728, Seq. ID No. 3746, Seq. ID No. 3754, Seq. ID No. 3756, Seq. ID No. 3758, Seq. ID No. 3762, Seq. ID No. 3764, Seq. ID No. 3766, Seq. ID No. 3784, Seq. ID No. 3794, Seq. ID No. 3800, Seq. ID No. 3814, Seq. ID No. 3838, Seq. ID No. 3840, Seq. ID No. 3850, Seq. ID No. 3852, Seq. ID No. 3854, Seq. ID No. 3860, Seq. ID No. 3862, Seq. ID No. 3864, Seq. ID No. 3866, Seq. ID No. 3880, Seq. ID No. 3890, Seq. ID No. 3892, Seq. ID No. 3904, Seq. ID No. 3926, Seq. ID No. 3964, Seq. ID No. 3974, Seq. ID No. 3976, Seq. ID No. 4006, Seq. ID No. 4026, Seq. ID No. 4028, Seq. ID No. 4040, Seq. ID No. 4042, Seq. ID No. 4044, Seq. ID No. 4046, Seq. ID No. 4048, Seq. ID No. 4062, Seq. ID No. 4072, Seq. ID No. 4074, Seq. ID No. 4080, Seq. ID No. 4082, Seq. ID No. 4084, Seq. ID No. 4102, Seq. ID No. 4104, Seq. ID No. 4112, Seq. ID No. 4134, Seq. ID No. 4138, Seq. ID No. 4140, Seq. ID No. 4146, Seq. ID No. 4152, Seq. ID No. 4222, Seq. ID No. 4224, Seq. ID No. 4226, Seq. ID No. 4228, Seq. ID No. 4232, Seq. ID No. 4242, Seq. ID No. 4248, Seq. ID No. 4254, Seq. ID No. 4258, Seq. ID No. 4262, Seq. ID No. 4308, Seq. ID No. 4312, Seq. ID No. 4314, Seq. ID No. 4344, Seq. ID No. 4380, Seq. ID No. 4386, Seq. ID No. 4390, Seq. ID No. 4440, Seq. ID No. 4470, Seq. ID No. 4474, Seq. ID No. 4482, Seq. ID No. 4484, Seq. ID No. 4486, Seq. ID No. 4498, Seq. ID No. 4516, Seq. ID No. 4534, Seq. ID No. 4588, Seq. ID No. 4640, Seq. ID No. 4644, Seq. ID No. 4682, Seq. ID No. 4706, Seq. ID No. 4708, Seq. ID No. 4710, Seq. ID No. 4712, Seq. ID No. 4716, Seq. ID No. 4718, Seq. ID No. 4738, Seq. ID No. 4760, Seq. ID No. 4814, Seq. ID No. 4828, Seq. ID No. 4870, Seq. ID No. 4872, Seq. ID No. 4874, Seq. ID No. 4876, Seq. ID No. 4920, Seq. ID No. 4932, Seq. ID No. 4950, Seq. ID No. 4952, Seq. ID No. 4988, Seq. ID No. 5032, Seq. ID No. 5062, Seq. ID No. 5086, Seq. ID No. 5104, Seq. ID No. 5124, Seq. ID No. 5130, Seq. ID No. 5132, Seq. ID No. 5136, Seq. ID No. 5138, Seq. ID No. 5142, Seq. ID No. 5166, Seq. ID No. 5176, Seq. ID No. 5200, Seq. ID No. 5206, Seq. ID No. 5208, Seq. ID No. 5214, Seq. ID No. 5220, Seq. ID No. 5224, Seq. ID No. 5268, Seq. ID No. 5282, Seq. ID No. 5386, Seq. ID No. 5388, Seq. ID No. 5414, Seq. ID No. 5416, Seq. ID No. 5434, Seq. ID No. 5454, Seq. ID No. 5460, Seq. ID No. 5462, Seq. ID No. 5464, Seq. ID No. 5466, Seq. ID No. 5492, Seq. ID No. 5498, Seq. ID No. 5510, Seq. ID No. 5514, Seq. ID No. 5526, Seq. ID No. 5532, Seq. ID No. 5570, Seq. ID No. 5588, Seq. ID No. 5596, Seq. ID No. 5598, Seq. ID No. 5610, Seq. ID No. 5612, Seq. ID No. 5622, Seq. ID No. 5624, Seq. ID No. 5634, Seq. ID No. 5638, Seq. ID No. 5644, Seq. ID No. 5650, Seq. ID No. 5660, Seq. ID No. 5684, Seq. ID No. 5686, Seq. ID No. 5700, Seq. ID No. 5706, Seq. ID No. 5710, Seq. ID No. 5730, Seq. ID No. 5734, Seq. ID No. 5764, Seq. ID No. 5766, Seq. ID No. 5768, Seq. ID No. 5772, Seq. ID No. 5784, Seq. ID No. 5792, Seq. ID No. 5798, Seq. ID No. 5886, Seq. ID No. 5890, Seq. ID No. 5894, Seq. ID No. 5896, Seq. ID No. 5900, Seq. ID No. 5902, Seq. ID No. 5906, Seq. ID No. 5910, Seq. ID No. 5922, Seq. ID No. 5924, Seq. ID No. 5928, Seq. ID No. 5940, Seq. ID No. 6028, Seq. ID No. 6044, Seq. ID No. 6048, Seq. ID No. 6060, Seq. ID No. 6068, Seq. ID No. 6090, Seq. ID No. 6092, Seq. ID No. 6108, Seq. ID No. 6112, Seq. ID No. 6118, Seq. ID No. 6162, Seq. ID No. 6164, Seq. ID No. 6166, Seq. ID No. 6170, Seq. ID No. 6184, Seq. ID No. 6186, Seq. ID No. 6190, Seq. ID No. 6192, Seq. ID No. 6198, Seq. ID No. 6204, Seq. ID No. 6206, Seq. ID No. 6210, Seq. ID No. 6222, Seq. ID No. 6224, Seq. ID No. 6226, Seq. ID No. 6228, Seq. ID No. 6230, Seq. ID No. 6236, Seq. ID No. 6238, Seq. ID No. 6244, Seq. ID No. 6246, Seq. ID No. 6250, Seq. ID No. 6254, Seq. ID No. 6264, Seq. ID No. 6266, Seq. ID No. 6274, Seq. ID No. 6276, Seq. ID No. 6286, Seq. ID No. 6290, Seq. ID No. 6302, Seq. ID No. 6304, Seq. ID No. 6364, Seq. ID No. 6378, Seq. ID No. 6382, Seq. ID No. 6384, Seq. ID No. 6416, Seq. ID No. 6428, Seq. ID No. 6432, Seq. ID No. 6474, Seq. ID No. 6498, Seq. ID No. 6572, Seq. ID No. 6582, Seq. ID No. 6594, Seq. ID No. 6600, Seq. ID No. 6632, and Seq. ID No. 6640.

56. An isolated polypeptide encoded by a polynucleotide comprising a nucleotide sequence having at least about 70% identity to a nucleotide sequence contained within SEQ ID NO: 6651, a complement thereof, a degenerate variant thereof, and a fragment thereof.

57. A recombinant expression vector comprising a nucleotide sequence having at least about 70% identity to a nucleotide sequence chosen from one of the odd numbered sequences set out in SEQ ID NO: 1 through SEQ ID NO: 6649, a complement thereof, a degenerate variant thereof, and a fragment thereof.

58. The vector of claim 57, wherein the polynucleotide is selected from the group consisting of genomic DNA, cDNA, and RNA.

59. The vector of claim 58, wherein the polynucleotide comprises heterologous nucleotide sequences.

60. The vector of claim 58, wherein the polynucleotide is operatively linked to one or more gene expression regulatory elements.

61. The vector of claim 60, wherein the polynucleotide encodes a polypeptide comprising an amino acid sequence having at least about 70% identity to an amino acid sequence chosen from one of even numbered sequences set out in SEQ ID NO: 2 through SEQ ID NO: 6650, a biological equivalent thereof, and a fragment thereof.

62. The vector of claim 57, wherein the vector is a plasmid.

63. A genetically engineered host cell, transfected, transformed or infected with the vector of claim 57.

64. The host cell of claim 63, wherein the host cell is a bacterial cell.

65. The host cell of claim 64, wherein the polynucleotide is expressed to produce the encoded polypeptide, a biological equivalent thereof, or a fragment thereof.

66. An antibody specific for a polypeptide encoded by an Alloiococcus otitidis polynucleotide chosen from one of the odd numbered sequences from SEQ ID NO: 1 through SEQ ID NO: 6649, a complement thereof, a fragment thereof, and a degenerate variant thereof.

67. An antibody specific for an Alloiococcus otitidis polypeptide chosen from one of even numbered sequence listings set out in SEQ ID NO: 2 through SEQ ID NO: 6650, a complement thereof, a biological equivalent thereof, and a fragment thereof.

68. The antibody of claim 66, wherein the antibody is selected from the group consisting of monoclonal, polyclonal, chimeric, humanized and single chain.

69. The antibody of claim 67, wherein the antibody is selected from the group consisting of monoclonal, polyclonal, chimeric, humanized and single chain.

70. The antibody of claim 66, wherein the antibody is monoclonal.

71. The antibody of claim 67, wherein the antibody is monoclonal.

72. The antibody of claim 66, wherein the antibody is humanized.

73. The antibody of claim 67, wherein the antibody is humanized.

74. An immunogenic composition comprising a polypeptide having an amino acid a sequence chosen from one of even numbered sequence listings set out in SEQ ID NO: 2 through SEQ ID NO: 6650, a complement thereof, a biological equivalent thereof, and a fragment thereof.

75. The immunogenic composition of claim 74, further comprising a pharmaceutically acceptable carrier.

76. The immunogenic composition of claim 74, further comprising one or more adjuvants.

77. The immunogenic composition of claim 74, wherein the polypeptide is further defined as:
(a) an Alloiococcus otitidis polypeptide predicted by SignalP algorithm analysis as having met all four criteria set out in the SignalP algorithm for a polypeptide having a signal peptide and predicted by HMM Signal P algorithm analysis as having a signal peptide;
(b) an Alloiococcus otitidis polypeptide predicted by SignalP algorithm analysis as having met three of the four criteria set out in the SignalP algorithm for a polypeptide having a signal peptide and predicted by HMM Signal P algorithm analysis as having a signal peptide;
(c) an Alloiococcus otitidis polypeptide predicted by HMM SignalP algorithm analysis as having a signal peptide;
(d) an Alloiococcus otitidis polypeptide predicted by HMM SignalP algorithm analysis as being a non-secretory protein;
(e) an Alloiococcus otitidis polypeptide identified by BlastP analysis;
(f) an Alloiococcus otitidis polypeptide identified by Pfam analysis;
(g) an Alloiococcus otitidis polypeptide identified using proteomics methodology as sharing homology with surface exposed proteins of Streptococcus pneumoniae;
(h) an Alloiococcus otitidis lipoprotein;
(i) an Alloiococcus otitidis polypeptide having a LPXTG motif, wherein the polypeptide is covalently attached to the peptidoglycan layer;
(j) an Alloiococcus otitidis polypeptide, wherein the polypeptide is non-covalently associated with the peptidoglycan layer;
(k) an Alloiococcus otitidis polypeptide having an RGDX motif wherein X is not a proline residue;
(l) an Alloiococcus otitidis polypeptide predicted by BlastP as being involved in capsule biosynthesis and transport;
(m) an Alloiococcus otitidis polypeptide identified by BlastP as being localized within the capsule loci of the Alloiococcus otitidis genome;
(n) an Alloiococcus otitidis polypeptide predicted by BlastP as being associated with sporulation;
(o) an Alloiococcus otitidis polypeptide encoded by unique ORFs identified by BlastP analysis as having a BlastP ‘E Value’ of >e⁻¹⁰;
(p) an Alloiococcus otitidis polypeptide identified by Glimmer™ ORF finder program;
(q) an Alloiococcus otitidis polypeptide identified by GeneMark™ ORF finder program;
(r) an Alloiococcus otitidis polypeptide identified by an ORF finder program that searches for an ATG, GTG or TTG start codon between a Stop-Stop region; and
(s) an Alloiococcus otitidis polypeptide identified by an ORF finder program that searches for a transmembrane domain between two Stop codons and a Stop codon immediately upstream of this transmembrane region.

78. The immunogenic composition of claim 77, wherein the polypeptide further comprises heterologous amino acids.

79. The immunogenic composition of claim 77, wherein the polypeptide is a fusion polypeptide.

80. The immunogenic composition of claim 77, wherein the polypeptide is encoded by a polynucleotide comprising a nucleotide sequence having at least about 70% identity to a nucleotide sequence chosen from one of odd numbered sequence listings set out in SEQ ID NO: 1 through SEQ ID NO: 6649, a degenerate variant thereof, or a fragment thereof.

81. The immunogenic composition of claim 8D, wherein the polynucleotide further comprises heterologous nucleotides.

82. An immunogenic composition comprising a polynucleotide having a nucleotide sequence chosen from one of one of odd numbered sequence listings set out in SEQ ID NO: 1 through SEQ ID NO: 6649, a degenerate variant thereof, or a fragment thereof and is comprised in an expression vector.

83. The immunogenic composition of claim 82, wherein the vector is plasmid DNA.

84. The immunogenic composition of claim 83, wherein the polynucleotide comprises heterologous nucleotides.

85. The immunogenic composition of claim 83, wherein the polynucleotide is operatively linked to one or more gene expression regulatory elements.

86. The immunogenic composition of claim 85, wherein the polynucleotide directs the expression of a neutralizing epitope of Alloiococcus otitidis.

87. The immunogenic composition of claim 86, further comprising one or more adjuvants.

88. A pharmaceutical composition comprising a polypeptide and a pharmaceutically acceptable carrier, wherein the polypeptide comprises an amino acid chosen from one of even numbered sequence listings set out in SEQ ID NO: 2 through SEQ ID NO: 6650, a complement thereof, a biological equivalent thereof, and a fragment thereof.

89. The pharmaceutical composition of claim 88, wherein the polypeptide is further defined as:
(a) an Alloiococcus otitidis polypeptide predicted by SignalP algorithm analysis as having met all four criteria set out in the SignalP algorithm for a polypeptide having a signal peptide and predicted by HMM SignalP algorithm analysis as having a signal peptide;
(b) an Alloiococcus otitidis polypeptide predicted by SignalP algorithm analysis as having met three of the four criteria set out in the SignalP algorithm for a polypeptide having a signal peptide and predicted by HMM SignalP algorithm analysis as having a signal peptide;
(c) an Alloiococcus otitidis polypeptide predicted by HMM SignalP algorithm analysis as having a signal peptide;
(d) an Alloiococcus otitidis polypeptide predicted by HMM SignalP algorithm analysis as being a non-secretory protein;
(e) an Alloiococcus otitidis polypeptide identified by BlastP analysis;
(f) an Alloiococcus otitidis polypeptide identified by Pfam analysis;
(g) an Alloiococcus otitidis polypeptide identified using proteomics methodology as sharing homology with surface exposed proteins of Streptococcus pneumoniae;
(h) an Alloiococcus otitidis lipoprotein;
(i) an Alloiococcus otitidis polypeptide having a LPXTG motif, wherein the polypeptide is covalently attached to the peptidoglycan layer;
(j) an Alloiococcus otitidis polypeptide, wherein the polypeptide is non-covalently associated with the peptidoglycan layer;
(k) an Alloiococcus otitidis polypeptide having an RGDX motif wherein X is not a proline residue;
(l) an Alloiococcus otitidis polypeptide predicted by BlastP as being involved in capsule biosynthesis and transport;
(m) an Alloiococcus otitidis polypeptide identified by BlastP as being localized within the capsule loci of the Alloiococcus otitidis genome;
(n) an Alloiococcus otitidis polypeptide predicted by BlastP as being associated with sporulation;
(o) an Alloiococcus otitidis polypeptide encoded by unique ORFs identified by BlastP analysis as having a BlastP ‘E Value’ of >e⁻¹⁰;
(p) an Alloiococcus otitidis polypeptide identified by Glimmer™ ORF finder program;
(q) an Alloiococcus otitidis polypeptide identified by GeneMark™ ORF finder program;
(r) an Alloiococcus otitidis polypeptide identified by an ORF finder program that searches for an ATG, GTG or TTG start codon between a Stop-Stop region; and
(s) an Alloiococcus otitidis polypeptide identified by an ORF finder program that searches for a transmembrane domain between two Stop codons and a Start codon immediately upstream of this transmembrane region.

90. The pharmaceutical composition of claim 89, wherein the polypeptide further comprises heterologous amino acids.

91. The pharmaceutical composition of claim 90, wherein the polypeptide is a fusion polypeptide.

92. A DNA chip comprising an array of polynucleotides, wherein at least one of the polynucleotides comprise a nucleotide sequence chosen from one of one of odd numbered sequence listings set out in SEQ ID NO: 1 through SEQ ID NO: 6649, a complement thereof, a degenerate variant thereof, and a fragment thereof.

93. A protein chip comprising an array of polypeptides, wherein at least one of the polypeptides comprises an amino acid sequence chosen from one of even numbered sequence listings set out in SEQ ID NO: 2 through SEQ ID NO: 6650, a biological equivalent thereof, or a fragment thereof.

94. A method of immunizing against Alloiococcus otitidis comprising administering to a host an immunizing amount of an immunogenic composition comprising a polypeptide and a pharmaceutically acceptable carrier, wherein the polypeptide comprises an amino acid sequence chosen from one of even numbered sequence listings set out in SEQ ID NO: 2 through SEQ ID NO: 6650, a biological equivalent thereof, or a fragment thereof.

95. The method of claim 94, wherein the polypeptide is a fusion polypeptide.

96. The method of claim 94, further comprising an adjuvant.

97. A method for the detection and/or identification of Alloiococcus otitidis in a biological sample comprising:
(a) contacting the sample with an oligonucleotide probe of a polynucleotide comprising the nucleotide sequence chosen from one of odd numbered sequence listings set out in SEQ ID NO: 1 through SEQ ID NO: 6649, a degenerate variant thereof, or a fragment thereof, under conditions permitting hybridization; and
(b) detecting the presence of hybridization complexes in the sample, wherein hybridization complexes indicate the presence of Alloiococcus otitidis in the sample.

98. A method for the detection and/or identification of Alloiococcus otitidis in a biological sample comprising:
(a) contacting the sample with an oligonucleotide primer of a polynucleotide comprising the nucleotide sequence chosen from one of odd numbered sequence listings set out in SEQ ID NO: 1 through SEQ ID NO: 6649, a degenerate variant thereof, or a fragment thereof, in the presence of nucleotides and a polymerase enzyme under conditions permitting primer extension; and
(b) detecting the presence of primer extension products in the sample, wherein extension products indicate the presence of Alloiococcus otitidis in the sample.

99. A method for the detection and/or identification of Alloiococcus otitidis in a biological sample comprising:
(a) contacting the sample with an antibody specific for a polypeptide comprising an amino acid sequence chosen from one of even numbered sequence listings set out in SEQ ID NO: 2 through SEQ ID NO: 6650, a biological equivalent thereof, or a fragment thereof, under conditions permitting immune complex formation; and
(b) detecting the presence of immune complexes in the sample, wherein immune complexes indicate the presence of Alloiococcus otitidis in the sample.

100. A method for the detection and/or identification of antibodies to Alloiococcus otitidis in a biological sample comprising:
(a) contacting the sample with a polypeptide comprising an amino acid sequence chosen from one of even numbered sequence listings set out in SEQ ID NO: 2 through SEQ ID NO: 6650, a biological equivalent thereof, Nor a fragment thereof, under conditions permitting immune complex formation; and
(b) detecting the presence of immune complexes in the sample, wherein immune complexes indicate the presence of Alloiococcus otitidis in the sample.

101. A kit comprising a container containing an isolated polynucleotide comprising an nucleotide sequence chosen from one of odd numbered sequence listing set out in SEQ ID NO: 1 through SEQ ID NO: 6649, a degenerate variant thereof, or a fragment thereof.

102. The kit of claim 101, wherein the polynucleotide is a primer or a probe.

103. The kit of claim 101, wherein the polynucleotide is a primer and the kit further comprises a container containing a polymerase.

104. The kit of claim 103, wherein the kit further comprises a container containing dNTP.

105. A kit comprising a container containing an antibody that immunospecifically binds to a polypeptide comprising the amino acid sequence chosen from one of even numbered sequence listings set out in SEQ ID NO: 2 through SEQ ID NO: 6650, a biological equivalent thereof, or a fragment thereof.

106. A kit comprising a container containing an antibody that immunospecifically binds to a fusion polypeptide comprising at least the amino acid sequence chosen from one of even numbered sequence listings set out in SEQ ID NO: 2 through SEQ ID NO: 6650, a biological equivalent thereof, or a fragment thereof.

107. A method for producing a polypeptide, which comprises culturing the genetically engineered host cell of claim 63 under conditions suitable to produce the polypeptide and recovering the polypeptide from the culture.
